SEQUENCE LISTING (1 sequence)

SEQ ID NO: 1 - protein, 785 aa, Artificial Sequence
  USP1 protein
MPGVIPSESNGLSRGSPSKKNRLSLKFFQKKETKRALDFTDSQENEEKASEYRASEIDQVVPAAQSSPINCEKRENLLPFVGLNNLGNTCYLNSILQVLYFCPGFKSGVKHLFNIISRKKEALKDEANQKDKGNCKEDSLASYELICSLQSLIISVEQLQASFLLNPEKYTDELATQPRRLLNTLRELNPMYEGYLQHDAQEVLQCILGNIQETCQLLKKEEVKNVAELPTKVEEIPHPKEEMNGINSIEMDSMRHSEDFKEKLPKGNGKRKSDTEFGNMKKKVKLSKEHQSLEENQRQTRSKRKATSDTLESPPKIIPKYISENESPRPSQKKSRVKINWLKSATKQPSILSKFCSLGKITTNQGVKGQSKENECDPEEDLGKCESDNTTNGCGLESPGNTVTPVNVNEVKPINKGEEQIGFELVEKLFQGQLVLRTRCLECESLTERREDFQDISVPVQEDELSKVEESSEISPEPKTEMKTLRWAISQFASVERIVGEDKYFCENCHHYTEAERSLLFDKMPEVITIHLKCFAASGLEFDCYGGGLSKINTPLLTPLKLSLEEWSTKPTNDSYGLFAVVMHSGITISSGHYTASVKVTDLNSLELDKGNFVVDQMCEIGKPEPLNEEEARGVVENYNDEEVSIRVGGNTQPSKVLNKKNVEAIGLLGGQKSKADYELYNKASNPDKVASTAFAENRNSETSDTTGTHESDRNKESSDQTGINISGFENKISYVVQSLKEYEGKWLLFDDSEVKVTEEKDFLNSLSPSTSPTSTPYLLFYKKL

(19) United States
(12) Patent Application Publication
(10) Pub. No.: US 2023/0219965 A1
(43) Pub. Date: Jul. 13, 2023

(54) NITROGEN-CONTAINING FUSED BICYCLIC COMPOUNDS AND THEIR USE AS UBIQUITIN-SPECIFIC-PROCESSING PROTEASE 1 (USP1) INHIBITORS

(71) Applicant: KSQ Therapeutics, Inc., Lexington, MA (US)

(72) Inventors: Jehrod Burnett BRENNEMAN, Marblehead, MA (US); Dominique BONAFOUX, Winthrop, MA (US); Jonh FEUTRILL, Rosanna, Victoria (AU)

(21) Appl. No.: 18/000,500

(22) PCT Filed: Jun. 1, 2021
(86) PCT No.: PCT/US2021/035289

§ 371 Date: Dec. 1, 2022

Related U.S. Application Data
(60) Provisional application No. 63/033,584, filed on Jun. 2, 2020.

(51) Int. Cl. C07D 487/04; C07D 471/04

(52) U.S. Cl. CPC C07D 487/04; C07D 471/04

(57) ABSTRACT

The present disclosure provides compounds having Formula (I): (I) and the pharmaceutically acceptable salts and solvates thereof, wherein X¹, X², X³, X⁴, X⁵, X⁶, X⁷, X⁸, R¹, R², R⁶, R^{6′}, R⁷, and R^{7′} are defined as set forth in the specification. The present disclosure is also directed to the use of compounds of Formula (I) to inhibit a USP1 protein and/or to treat a disorder responsive to the inhibition of USP1 proteins and USP1 activity. Compounds of the present disclosure are especially useful for treating cancer.

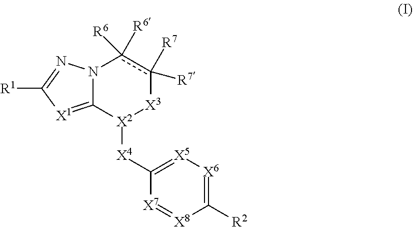

### REFERENCE TO SEQUENCE LISTING SUBMITTED ELECTRONICALLY

The content of the electronically submitted sequence listing (Name: 4195_021PC01-sequence-listing.txt; Size: 7,204 Bytes; and Date of Creation: Jun. 1, 2021) is herein incorporated by reference in its entirety.

### BACKGROUND OF THE DISCLOSURE

### Field of the Invention

The present disclosure provides nitrogen-containing fused bicyclic compounds as ubiquitin-specific-processing protease 1 (USP1) inhibitors, and therapeutic methods of treating conditions and diseases wherein inhibition of USP1 provides a benefit. In particular, the present disclosure provides methods of treating cancer by administering a USP1 inhibitor.

### Background

Ubiquitin is a small (76 amino acid) protein that is post-transcriptionally attached to target proteins. The consequence of ubiquitination is determined by the number and linkage topology of ubiquitin molecules conjugated to the target protein. For example, proteins exhibiting lysine 48-linked poly-ubiquitin chains are generally targeted to the proteasome for degradation, while mono-ubiquitination or poly-ubiquitin chains linked through other lysines regulate non-proteolytic functions, such as cell cycle regulation, DNA damage repair, transcription, and endocytosis. Ubiquitination is a reversible process, and enzymes called deubiquitinases remove ubiquitin from target proteins.

USP1 is a deubiquitinase that plays a role in DNA damage repair. USP1 interacts with UAF1 (USP1-associated factor 1) to form a complex that is required for the deubiquitinase activity. The USP1/UAF1 complex deubiquitinates mono-ubiquitinated PCNA (proliferating cell nuclear antigen) and mono-ubiquitinated FANCD2 (Fanconi anemia group complementation group D2), which are proteins that play important functions in translesion synthesis (TLS) and the Fanconi anemia (FA) pathway, respectively. The USP1/UAF1 complex also deubiquitinates Fanconi anemia complementation group I (FANCI). These two pathways are essential for repair of DNA damage induced by DNA cross-linking agents, such as cisplatin and mitomycin C (MMC).

Safe and effective treatments targeting deubiquitinases are unknown, not yet commercially available, or have not yet been clinically developed.

### BRIEF SUMMARY OF THE DISCLOSURE

In one aspect, the present disclosure relates to compounds, or a pharmaceutically acceptable salt or solvate thereof, having Formula I (also referred to herein as

Compounds of the Disclosure):

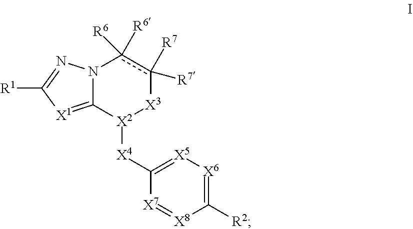

wherein:

X¹ is selected from N and CR⁵;

X² is selected from N and CH;

X³ is selected from NR³, O, and CR³R^{3′};

X⁴ is selected from NR⁴, O, and CR⁴R^{4′};

each of X⁵, X⁶, X⁷, and X⁸ are independently selected from N and CR⁸;

R¹ is selected from optionally substituted C₆ aryl and optionally substituted 5- or 6-membered nitrogen-containing heteroaryl;

R² is selected from hydrogen, halo, alkoxy, optionally substituted (C₆-C₁₄) aryl, optionally substituted (C₆-C₁₄) ar-(C₁-C₂) alkyl, optionally substituted nitrogen-containing heteroaryl, optionally substituted, nitrogen-containing heteroar-(C₁-C₂) alkyl, optionally substituted (C₃-C₈) cycloalkyl, optionally substituted ((C₃-C₈) cycloalkyl)-(C₁-C₂) alkyl, optionally substituted heterocyclo, optionally substituted heterocyclo-(C₁-C₂) alkyl, optionally substituted (C₆-C₁₄) aryloxy, optionally substituted (C₆-C₁₄) ar-(C₁-C₂) alkyloxy, optionally substituted nitrogen-containing heteroaryloxy, optionally substituted nitrogen-containing heteroar-(C₁-C₂) alkyloxy, optionally substituted (C₃-C₈) cycloalkyloxy, optionally substituted ((C₃-C₈) cycloalkyl)-(C₁-C₂) alkyloxy, optionally substituted heterocyclooxy, optionally substituted heterocyclo-(C₁-C₂) alkyloxy;

each of R³ and R^{3′} is independently selected from hydrogen, halo, cyano, amino, hydroxy, alkoxy, optionally substituted alkyl, optionally substituted alkenyl, and optionally substituted alkynyl, and optionally substituted cycloalkyl; or

R³ and R^{3′} are taken together with the atom to which they are attached to form a carbonyl or an optionally substituted cycloalkyl;

each of R⁴ and R^{4′} is independently selected from hydrogen, halo, cyano, amino, optionally substituted alkyl, optionally substituted alkenyl, and optionally substituted alkynyl, and optionally substituted cycloalkyl;

R⁵ is selected from hydrogen, halo, cyano, amino, alkylamino, dialkylamino, hydroxy, alkoxy, optionally substituted alkyl, optionally substituted alkenyl, and optionally substituted alkynyl, and optionally substituted cycloalkyl;

each of R⁶ and R^{6′}, if present, is independently selected from hydrogen, halo, cyano, amino, alkylamino, dialkylamino, hydroxy, alkoxy, optionally substituted alkyl, optionally substituted alkenyl, and optionally substituted alkynyl, and optionally substituted cycloalkyl, and alkylcarbonylamino;

each of R⁷ and R^{7′}, if present, is independently selected from hydrogen, halo, cyano, amino, alkylamino, dialkylamino, hydroxy, alkoxy, optionally substituted alkyl, optionally substituted alkenyl, and optionally substituted alkynyl, optionally substituted cycloalkyl, and alkylcarbonylamino; or

R⁷ and R^{7′} are taken together with the atom to which they are attached to form a carbonyl or an optionally substituted cycloalkyl; and

R⁸ is selected from hydrogen, halo, cyano, amino, alkylamino, dialkylamino, hydroxy, and alkoxy; or

R⁸ and R⁴ or R^{4′} are taken together with the atoms to which they are attached to form an optionally substituted cycloalkyl.

In some embodiments, each of R³ and R^{3′} is independently selected from hydrogen, halo, cyano, amino, hydroxy, (C₁-C₄) alkoxy, optionally substituted (C₁-C₄) alkyl, optionally substituted (C₂-C₄) alkenyl, and optionally substituted (C₂-C₄) alkynyl, and optionally substituted (C₃-C₈) cycloalkyl; or

R³ and R^{3′} are taken together with the atom to which they are attached to form a carbonyl or an optionally substituted (C₃-C₈) cycloalkyl.

In some embodiments, each of R⁴ and R^{4′} is independently selected from hydrogen, halo, cyano, amino, optionally substituted (C₁-C₄) alkyl, optionally substituted (C₂-C₄) alkenyl, and optionally substituted (C₂-C₄) alkynyl, and optionally substituted (C₃-C₈) cycloalkyl.

In some embodiments, R¹ is selected from hydrogen, halo, cyano, amino, (C₁-C₄) alkylamino, di-(C₁-C₄) alkylamino, hydroxy, (C₁-C₄) alkoxy, optionally substituted (C₁-C₄) alkyl, optionally substituted (C₂-C₄) alkenyl, and optionally substituted (C₂-C₄) alkynyl, and optionally substituted (C₃-C₈) cycloalkyl.

In some embodiments, each of R⁶ and R^{6′}, if present, is independently selected from hydrogen, halo, cyano, amino, (C₁-C₄) alkylamino, di-(C₁-C₄) alkylamino, hydroxy, (C₁-C₄) alkoxy, optionally substituted (C₁-C₄) alkyl, optionally substituted (C₂-C₄) alkenyl, and optionally substituted (C₂-C₄) alkynyl, and optionally substituted (C₃-C₈) cycloalkyl, and (C₁-C₄) alkylcarbonylamino.

In some embodiments, each of R¹ and IC, if present, is independently selected from hydrogen, halo, cyano, amino, (C₁-C₄) alkylamino, di-(C₁-C₄) alkylamino, hydroxy, (C₁-C₄) alkoxy, optionally substituted (C₂-C₄) alkyl, optionally substituted (C₂-C₄) alkenyl, and optionally substituted (C₂-C₄) alkynyl, optionally substituted (C₃-C₈) cycloalkyl, and (C₁-C₄) alkylcarbonylamino; or

R⁷ and R^{7′} are taken together with the atom to which they are attached to form a carbonyl or an optionally substituted (C₃-C₈) cycloalkyl.

In some embodiments, R⁸ is selected from hydrogen, halo, cyano, amino, (C₁-C₄) alkylamino, di-(C₁-C₄) alkylamino, hydroxy, and (C₁-C₄) alkoxy; or

R⁸ and R⁴ or R^{4′} are taken together with the atoms to which they are attached to form an optionally substituted (C₃-C₈) cycloalkyl.

In some embodiments,

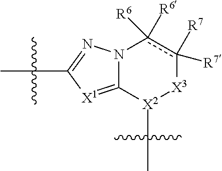

is independently selected from

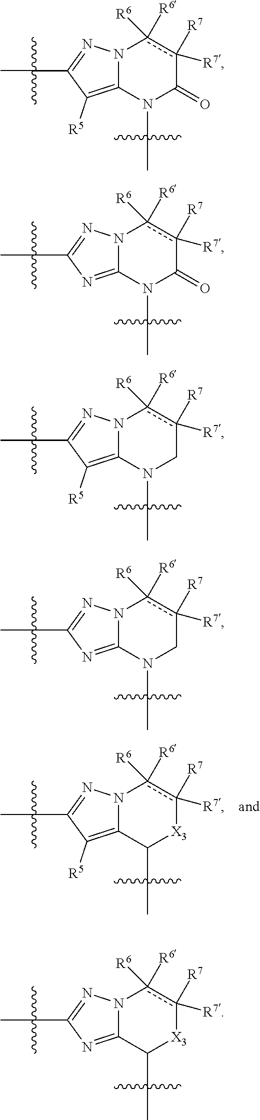

In some embodiments,

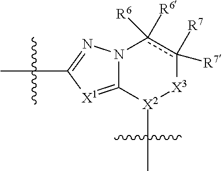

is independently selected from

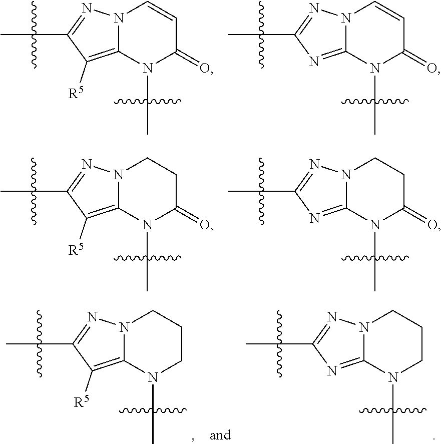

In some embodiments, X⁴ is CR⁴R^{4′} and X² is N.

In some embodiments, X⁴ is NR⁴ and X² is CH.

In some embodiments, one of X⁵, X⁶, X⁷, and X⁸ is N.

In some embodiments, two of X⁵, X⁶, X⁷, and X⁸ are N.

In some embodiments, X⁶ is CR⁸.

In some embodiments, R⁸ is selected from hydrogen and fluoro.

In some embodiments, the optional substituents on R¹ are independently selected from hydrogen, halo, nitro, cyano, hydroxy, amino, alkylamino, dialkylamino, haloalkyl, hydroxyalkyl, alkoxy, haloalkoxy, aryloxy, heteroaryloxy, aralkyl aralkyloxy, alkylthio, carboxamido, sulfonamido, alkylcarbonyl, arylcarbonyl, alkyl sulfonyl, aryl sulfonyl, carboxy, carboxyalkyl, alkyl, optionally substituted cycloalkyl, alkenyl, alkynyl, optionally substituted aryl, optionally substituted heteroaryl, optionally substituted heterocyclo, alkoxyalkyl, (amino)alkyl, hydroxyalkylamino, (alkylamino)alkyl, (dialkylamino)alkyl, (cyano)alkyl, (carboxamido)alkyl, mercaptoalkyl, (heterocyclo)alkyl, (cycloalkylamino)alkyl, (C₁₋₄ haloalkoxy)alkyl, and (heteroaryl)alkyl.

In some embodiments, two of the optional substituents on R¹ are taken together with the carbon or nitrogen atoms to which they are attached to form an optionally substituted cycloalkyl, optionally substituted heterocyclo, optionally substituted aryl, or optionally substituted heteroaryl group.

In some embodiments, R¹ is an optionally substituted 5- or 6-membered nitrogen-containing heteroaryl, wherein nitrogen is the only heteroatom.

In some embodiments, R¹ is an optionally substituted pyrimidin-5-yl.

In some embodiments, R¹ is an optionally substituted pyrimidin-5-yl, and wherein the pyrimidin-5-yl is optionally substituted at the 4-position, optionally substituted at the 6-position, optionally disubstituted at the 4- and 6-positions, or optionally trisubstituted at the 2-, 4-, and 6-positions.

In some embodiments, R¹ is an optionally substituted pyrazol-5-yl.

In some embodiments, R¹ is an optionally substituted pyrazol-5-yl, wherein the pyrazol-5-yl is optionally substituted at the 1-position, optionally substituted at the 4-position, or optionally disubstituted at the 1- and 4-positions.

In some embodiments, R¹ is an optionally substituted pyrid-3-yl or optionally substituted pyrid-4-yl.

In some embodiments, R¹ is an optionally substituted pyrid-3-yl or optionally substituted pyrid-4-yl, wherein the pyrid-3-yl is optionally substituted at the 2-position, optionally substituted at the 4-position, or optionally disubstituted at the 2- and 4-positions; and wherein the pyrid-4-yl is optionally substituted at the 3-position, optionally substituted at the 5-position, or optionally disubstituted at the 3- and 5-positions.

In some embodiments, R¹ is an optionally substituted phenyl.

In some embodiments, R¹ is an optionally substituted phenyl, wherein the phenyl is optionally substituted at the 2-position, optionally substituted at the 6-position, optionally disubstituted at the 2- and 6-positions, or optionally disubstituted at the 2- and 3-positions.

In some embodiments, R¹ is substituted.

In some embodiments, R¹ is substituted and the substituents are independently selected from hydrogen, cyano, amino, methylamino, dimethylamino, methoxy, ethoxy, isopropoxy, tert-butoxy, difluoromethoxy, trifluoromethoxy, cyclopropoxy, cyclobutoxy, methyl, ethyl, propyl, isopropyl, butyl, isobutyl, difluoromethyl, trifluoromethyl, cyclopropyl, cyclobutyl, isopropyl, tert-butyl, chloro, fluoro, 1-fluoropropan-2-yl, (5)-1-fluoropropan-2-yl, (R)-1-fluoropropan-2-yl, hydroxyethyl, 1-methoxy-2-methylpropan-2-yl, 1-methoxypropan-2-yl, (5)-1-methoxypropan-2-yl, (R)-1-methoxypropan-2-yl, 1-(methoxymethyl)cyclopropyl, 1-hydroxypropan-2-yl, oxetan-3-yl, tetrahydrofuran-3-yl, 1-methylcyclopropyl, deuteromethyl, deuteroethyl, deuteroisopropyl, deuteromethoxy, and deuteroethoxy.

In some embodiments, R¹ is independently selected from:

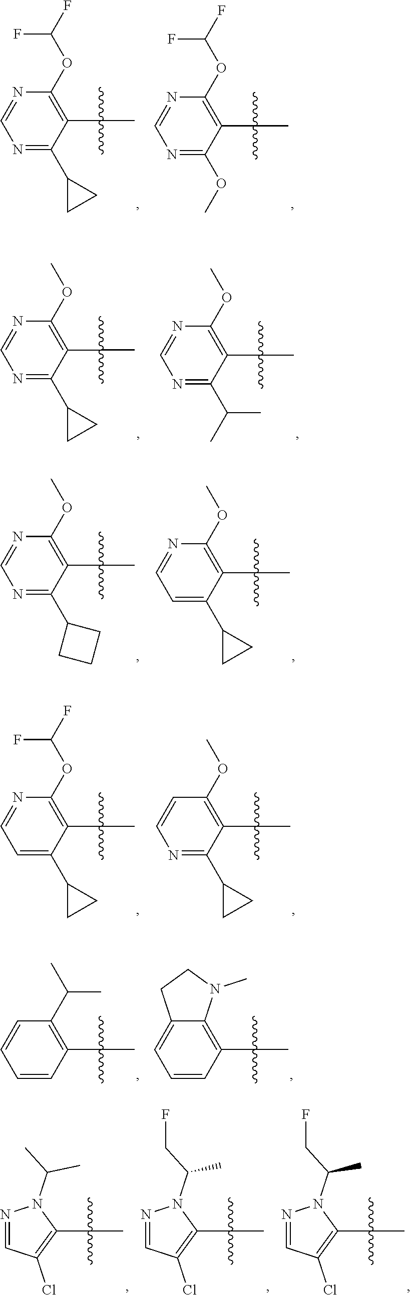

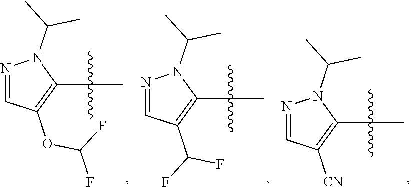

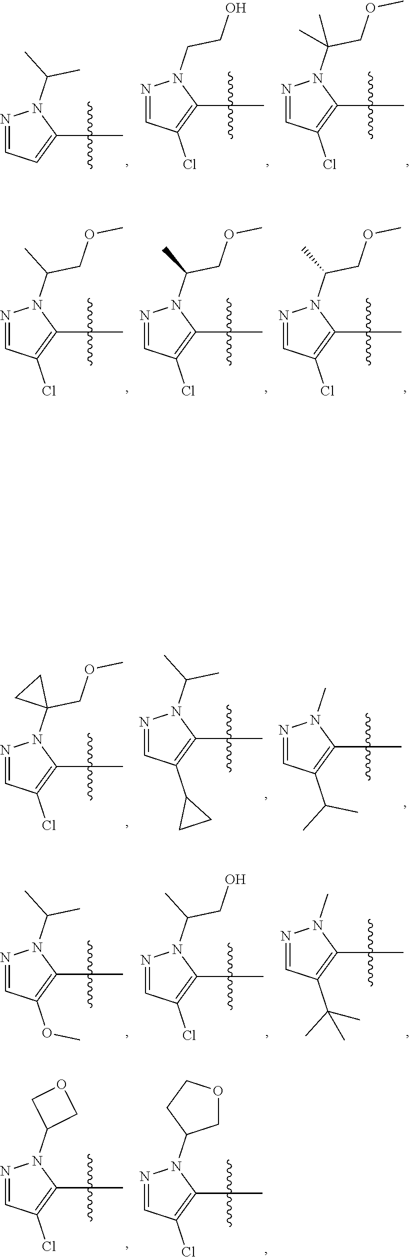

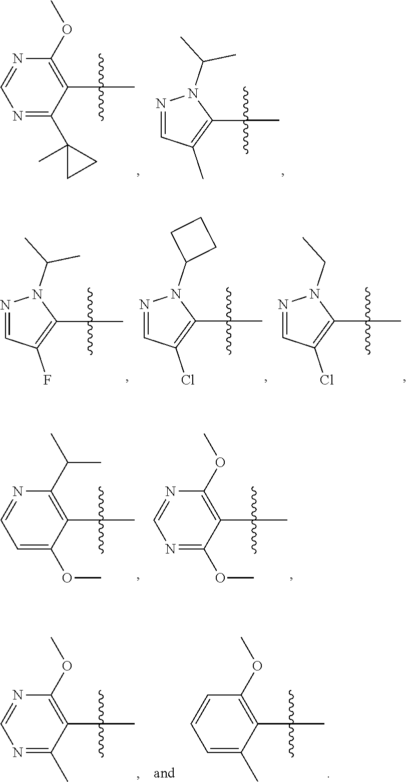

In some embodiments, the optional substituents on R² are independently selected from hydrogen, halo, nitro, cyano, hydroxy, amino, alkylamino, dialkylamino, haloalkyl, hydroxyalkyl, alkoxy, haloalkoxy, aryloxy, heteroaryloxy, optionally substituted cycloalkyloxy, aralkyl aralkyloxy, alkylthio, carboxamido, sulfonamido, alkylcarbonyl, arylcarbonyl, alkyl sulfonyl, arylsulfonyl, carboxy, carboxyalkyl, alkyl, optionally substituted cycloalkyl, alkenyl, alkynyl, optionally substituted aryl, optionally substituted heteroaryl, optionally substituted heterocyclo, alkoxyalkyl, (amino)alkyl, hydroxyalkylamino, (alkylamino)alkyl, (dialkylamino)alkyl, (cyano)alkyl, (carboxamido)alkyl, mercaptoalkyl, (heterocyclo)alkyl, (cycloalkylamino)alkyl, (C₁₋₄ haloalkoxy)alkyl, (heteroaryl)alkyl, and sulfonamide.

In some embodiments, two of the optional substituents on R² are taken together with the carbon or nitrogen atoms to which they are attached to form an optionally substituted cycloalkyl, optionally substituted heterocyclo, optionally substituted aryl, or optionally substituted heteroaryl group.

In some embodiments, R² is an optionally substituted 5- or 6-membered nitrogen-containing heteroaryl, wherein nitrogen is the only heteroatom.

In some embodiments, R² is an optionally substituted imidazolyl.

In some embodiments, R² is an optionally substituted pyridyl.

In some embodiments, R² is an optionally substituted pyrazolyl.

In some embodiments, R² is an optionally substituted pyridazinyl.

In some embodiments, R² is an optionally substituted pyrimidinyl.

In some embodiments, R² is an optionally substituted triazinyl.

In some embodiments, R² is an optionally substituted pyrazinyl.

In some embodiments, R² is an optionally substituted triazolyl.

In some embodiments, R² is substituted and the substituents are independently selected from hydrogen, cyano, nitro, fluoro, chloro, bromo, methyl, ethyl, propyl, isopropyl, butyl, isobutyl, tert-butyl, trifluoromethyl, methoxy, ethoxy, isopropoxy, tert-butoxy, difluoromethoxy, trifluoromethoxy, 1-fluoropropan-2-yl, 2-fluoroethyl, amino, methylamino, ethylamino, dimethylamino, 2,2-difluoroethoxy, cyclopropoxy, morpholino, methoxymethyl, N,N-dimethylsulfonamido, cyclopropyl, methylaminomethyl, deuteromethyl, deuteroethyl, deuteroisopropyl, deuteromethoxy, and deuteroethoxy.

In some embodiments, R² is independently selected from:

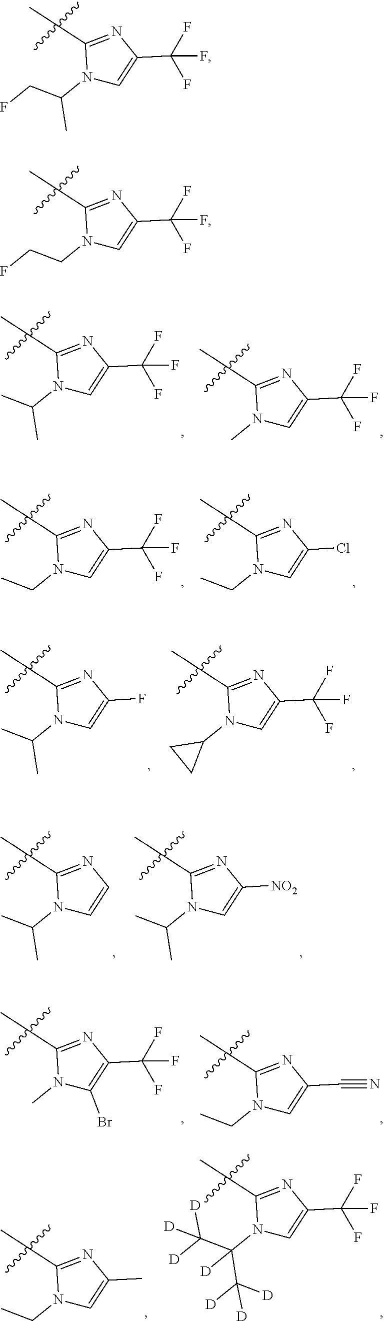

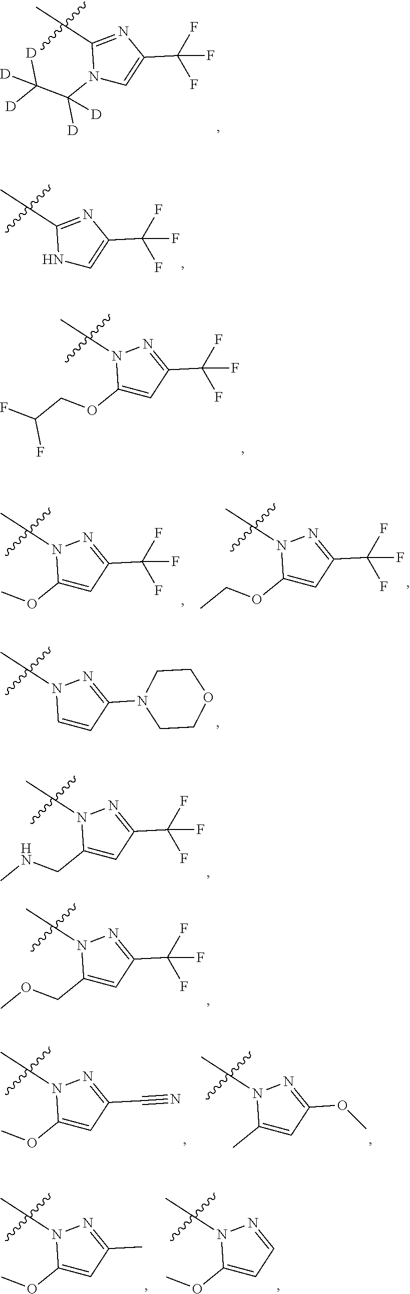

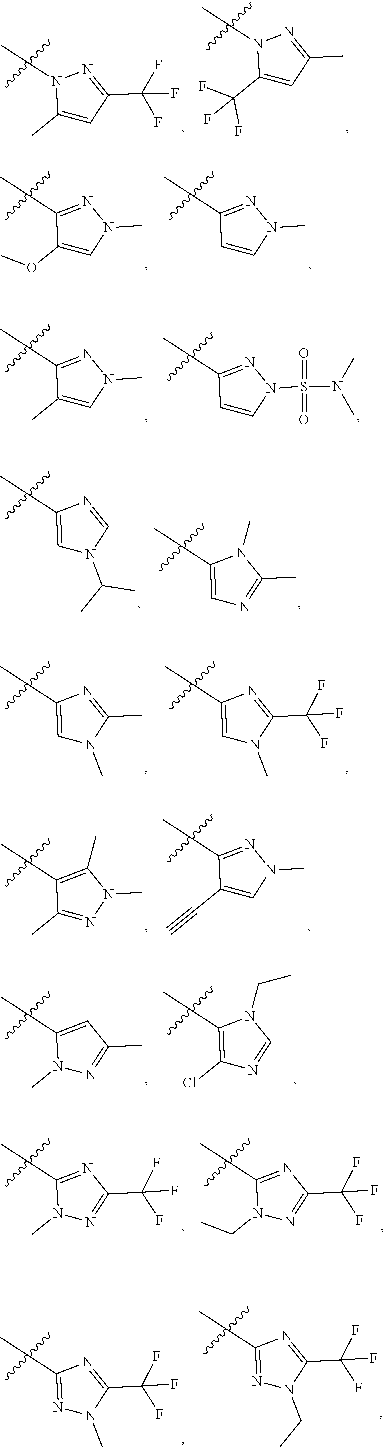

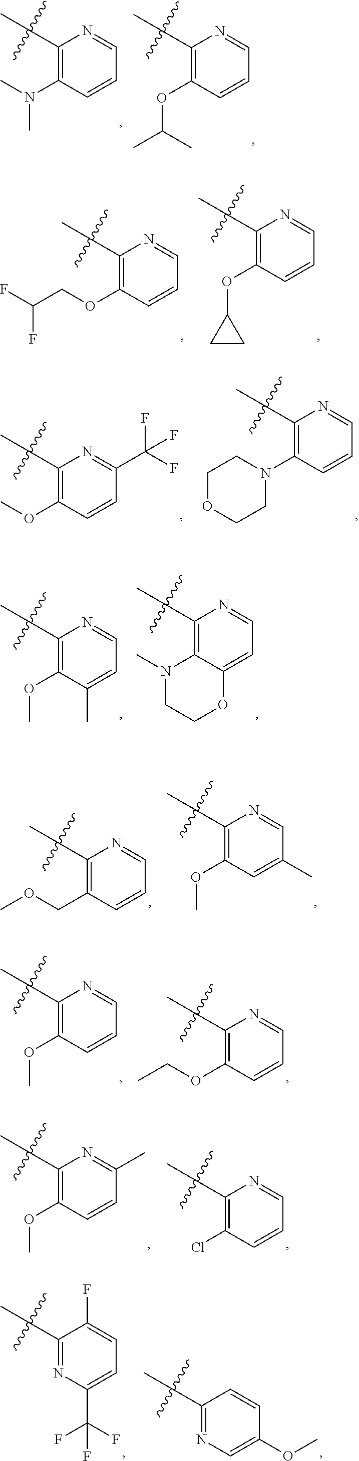

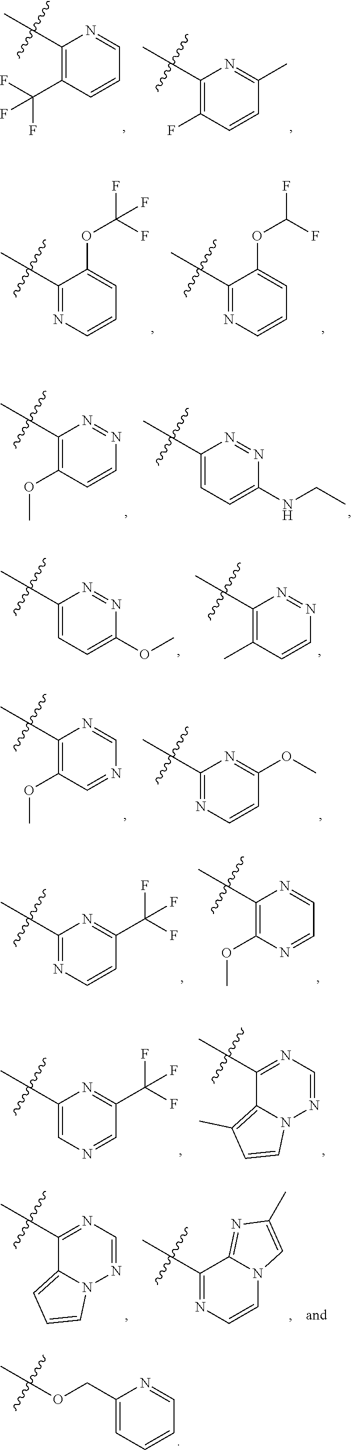

In some embodiments,

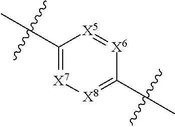

is independently selected from

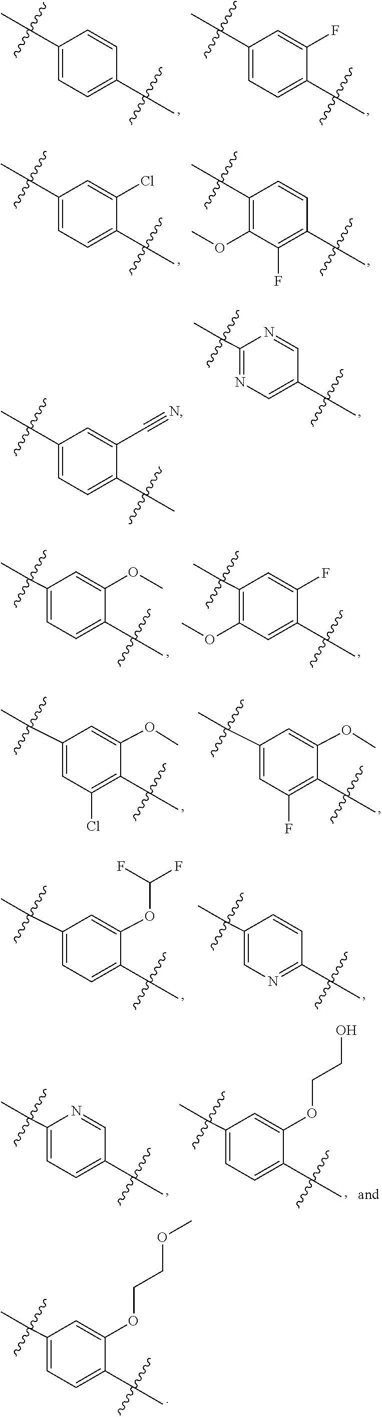

In some embodiments, the compound has Formula II, Formula III, Formula IV, Formula V, Formula VI, or Formula VII:

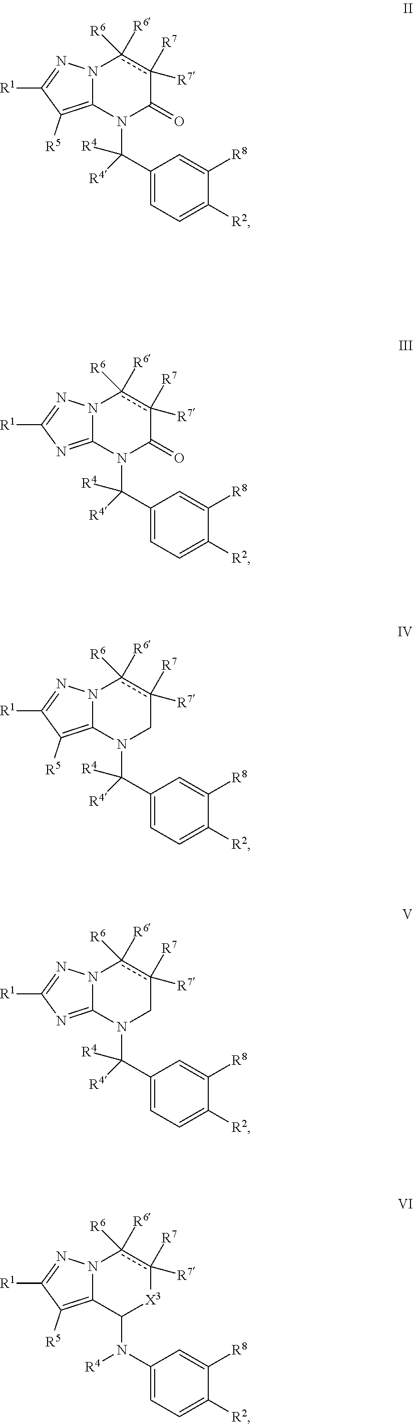

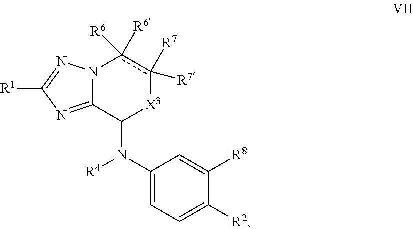

wherein:

R¹ is selected from

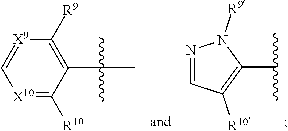

R² is selected from

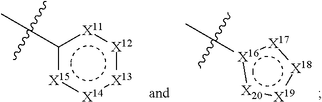

R⁴ and R^{4′} are independently selected from hydrogen, halo, and (C₁-C₃) alkyl; and

R⁵ is selected from hydrogen, halo, cyano, amino, alkylamino, dialkylamino, and (C₁-C₃) alkyl;

X⁹ is selected from N and CH;

X¹⁰ is selected from N and CH;

X¹¹ is selected from N and CR¹¹;

X¹² is selected from N and CR¹²;

X¹³ is selected from N and CR¹³;

X¹⁴ is selected from N and CR¹⁴;

X¹⁵ is selected from N and CR¹⁵;

X¹⁶ is selected from N and CR¹⁶;

X¹⁷ is selected from N and CR¹⁷;

X¹⁸ is selected from N and CR¹⁸;

X¹⁹ is selected from N and CR¹⁹;

X²⁰ is selected from N and CR²⁰,

each of R⁹, R^{9′}, R¹⁰, R^{10′}, R¹¹, R¹², R¹³, R¹⁴, R¹⁵, R¹⁶, R¹⁷, R¹⁸, R¹⁹, R²⁰ is independently selected from the group consisting of hydrogen, halo, nitro, cyano, hydroxy, amino, alkylamino, dialkylamino, haloalkyl, hydroxyalkyl, alkoxy, haloalkoxy, aryloxy, heteroaryloxy, aralkyl aralkyloxy, alkylthio, carboxamido, sulfonamido, alkylcarbonyl, arylcarbonyl, alkyl sulfonyl, arylsulfonyl, carboxy, carboxyalkyl, alkyl, optionally substituted cycloalkyl, alkenyl, alkynyl, optionally substituted aryl, optionally substituted heteroaryl, optionally substituted heterocyclo, alkoxyalkyl, (amino)alkyl, hydroxyalkylamino, (alkylamino)alkyl, (dialkylamino)alkyl, (cyano)alkyl, (carboxamido)alkyl, mercaptoalkyl, (heterocyclo)alkyl, (cycloalkylamino)alkyl, (C₁₋₄ haloalkoxy)alkyl, (heteroaryl)alkyl, or sulfonamide; or

wherein two of R¹¹, R¹², R¹³, R¹⁴, and R¹⁵ or two of R¹⁶, R¹⁷, R¹⁸, R¹⁹, and R²⁰ are taken together with the atom to which they are attached to form an optionally substituted cycloalkyl, optionally substituted heterocyclo, optionally substituted heteroaryl, or optionally substituted aryl.

In some embodiments, the Compound of the Disclosure has Formula VIII, or Formula IX

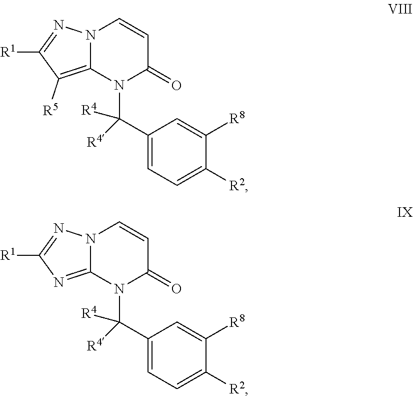

wherein:

R⁴ and R^{4′} are independently selected from hydrogen and methyl; and

R⁸ is selected from hydrogen and fluoro.

In some embodiments, the Compound of the Disclosure has Formula X, or Formula XI

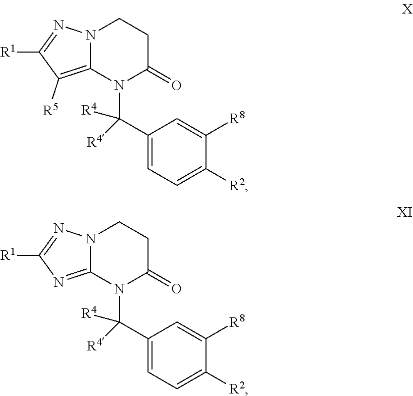

wherein:

R⁴ and R^{4′} are independently selected from hydrogen and methyl; and

R⁸ is selected from hydrogen and fluoro.

In some embodiments, the Compound of the Disclosure has Formula XII, or Formula XIII

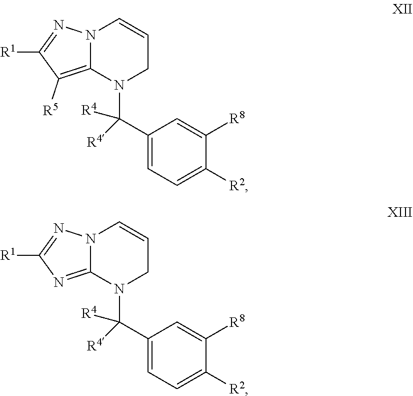

wherein:

R⁴ and R^{4′} are independently selected from hydrogen and methyl; and

R⁸ is selected from hydrogen and fluoro.

In some embodiments, the Compound of the Disclosure has Formula XIV, or Formula XV

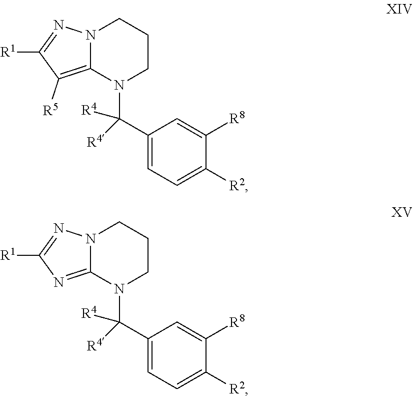

wherein:

R⁴ and R^{4′} are independently selected from hydrogen and methyl; and

R⁸ is selected from hydrogen and fluoro.

In some embodiments, certain Compounds of the Disclosure exhibit favorable solubility, e.g., as measured by an ADME solubility assay as disclosed herein, compared to compounds disclosed in the art as USP1 inhibitors.

In some embodiments, certain Compounds of the Disclosure exhibit favorable metabolic stability, e.g., as measured by liver microsome and hepatocyte metabolic stability assays as disclosed herein, compared to compounds disclosed in the art as USP1 inhibitors.

In other embodiments, certain Compounds of the Disclosure exhibit favorable duration of action and oral exposure in vivo.

In some embodiments, the Compound of the Disclosure is one of the specific compounds listed in the detailed description, or a pharmaceutically acceptable salt or solvate thereof.

In some embodiments, the Compound of the Disclosure inhibits a USP1 protein.

In some embodiments, the Compound of the Disclosure inhibits a USP1 protein with an IC₅₀ value of less than about 1 μM in a Ub-Rho deubiquitinating assay.

In some embodiments, the Ub-Rho deubiquitinating assay is the assay disclosed in Example 394.

In one aspect, the present disclosure relates to a method of treating cancer in a patient comprising administering to the patient a therapeutically effective amount of a Compound of the Disclosure, or a pharmaceutically acceptable salt or solvate thereof.

In one aspect, the present disclosure relates to a pharmaceutical composition comprising a Compound of the Disclosure, or a pharmaceutically acceptable salt or solvate thereof, and a pharmaceutically acceptable carrier.

In some embodiments, the present disclosure relates to a pharmaceutical composition for use in treatment of cancer.

In some embodiments, the present disclosure relates to a Compound of the Disclosure for use in treatment of cancer.

In some embodiments, the present disclosure relates to a use of a Compound of the Disclosure for the manufacture of a medicament for treatment of cancer.

In one aspect, the present disclosure relates to a kit comprising a Compound of the Disclosure, or a pharmaceutically acceptable salt or solvate thereof, or a pharmaceutical composition comprising a Compound of the Disclosure, and instructions for administering the compound, or a pharmaceutically acceptable salt or solvate thereof, or the pharmaceutical composition to a patient having cancer.

In one aspect, the present disclosure relates to a method of treating cancer in a patient comprising administering to the patient a Compound of the Disclosure, or a pharmaceutically acceptable salt or solvate thereof, or a pharmaceutical composition comprising a Compound of the Disclosure.

In some embodiments, the cancer is selected from the group consisting of a hematological cancer, a lymphatic cancer, and a DNA damage repair pathway deficient cancer, and a homologous-recombination deficient cancer.

In some embodiments, the cancer comprises cancer cells with a mutation in a gene encoding p53. In some embodiments, the mutation in a gene encoding p53 is a germline mutation. In some embodiments, the mutation in a gene encoding p53 is a somatic mutation. In some embodiments, the cancer comprises cancer cells with a loss of function mutation in a gene encoding p53.

In some embodiments, the cancer is selected from the group consisting of lung cancer, non-small cell lung cancer (NSCLC), colon cancer, bladder cancer, osteosarcoma, ovarian cancer, and breast cancer.

In some embodiments, the cancer is non-small cell lung cancer (NSCLC).

In some embodiments, the cancer is colon cancer.

In some embodiments, the cancer is bladder cancer.

In some embodiments, the cancer is ovarian cancer or breast cancer.

In some embodiments, the cancer is ovarian cancer.

In some embodiments, the cancer is breast cancer.

In some embodiments, the cancer is triple negative breast cancer.

In some embodiments, the cancer comprises cancer cells with elevated levels of RAD18.

In some embodiments, the elevated levels of RAD18 are elevated RAD18 protein levels.

In some embodiments, the elevated levels of RAD18 are elevated RAD18 mRNA levels.

In some embodiments, the elevated levels of RAD18 have been detected prior to the administration.

In another aspect, the present disclosure relates to a method that further comprises detecting RAD18 levels in a cancer sample obtained from the subject.

In some embodiments, the cancer is selected from the group consisting of bone cancer, including osteosarcoma and chondrosarcoma; brain cancer, including glioma, glioblastoma, astrocytoma, medulloblastoma, and meningioma; soft tissue cancer, including rhabdoid and sarcoma; kidney cancer; bladder cancer; skin cancer, including melanoma; and lung cancer, including non-small cell lung cancer; colon cancer, uterine cancer; nervous system cancer; head and neck cancer; pancreatic cancer; and cervical cancer.

In some embodiments, the cancer is a DNA damage repair pathway deficient cancer.

In some embodiments, the cancer comprises cancer cells with a mutation in a gene encoding p53. In some embodiments, the mutation in a gene encoding p53 is a germline mutation. In some embodiments, the mutation in a gene encoding p53 is a somatic mutation. In some embodiments, the cancer comprises cancer cells with a loss of function mutation in a gene encoding p53.

In some embodiments, the cancer is a BRCA1 mutant cancer. In some embodiments, the BRCA1 mutation is a germline mutation. In some embodiments, the BRCA1 mutation is a somatic mutation. In some embodiments, the BRCA1 mutation leads to BRCA1 deficiency.

In some embodiments, the cancer is a BRCA2 mutant cancer. In some embodiments, the BRCA2 mutation is a germline mutation. In some embodiments, the BRCA2 mutation is a somatic mutation. In some embodiments, the BRCA2 mutation leads to BRCA2 deficiency.

In some embodiments, the cancer is a BRCA1 mutant cancer and a BRCA2 mutant cancer.

In some embodiments, the cancer is a BRCA1 deficient cancer.

In some embodiments, the cancer is a BRCA2 deficient cancer.

In some embodiments, the cancer is a BRCA1 deficient cancer and a BRCA2 deficient cancer.

In some embodiments, the BRCA1 or BRCA2 mutant cancer is a BRCA1 or BRCA2 deficient cancer.

In some embodiments, the cancer is a Poly (ADP-ribose) polymerase (“PARP”) inhibitor refractory or resistant cancer. In some embodiments, the cancer is a PARP inhibitor resistant or refractory BRCA1, BRCA2, or BRCA1 and BRCA2 mutant cancer. In some embodiments, the cancer is a PARP inhibitor resistant or refractory BRCA1, BRCA2, or BRCA1 and BRCA2-deficient cancer.

In some embodiments, the cancer has a mutation in the gene encoding ataxia telangiectasia mutated (ATM) protein kinase. In some embodiments the ATM mutation is a germline mutation. In some embodiments the ATM mutation is a somatic mutation. In some embodiments the cancer is an ATM-deficient cancer.

In some embodiments the cancer has a mutation in the gene encoding at least two of p53, BRCA1, BRCA2, and ATM.

In another aspect, the present disclosure relates to a method of treating a USP1 protein mediated disorder comprising administering to a patient in need thereof a Compound of the Disclosure, or a pharmaceutically acceptable salt or solvate thereof, or a pharmaceutical composition comprising a Compound of the Disclosure, in an effective amount to treat the USP1 protein mediated disorder.

In some embodiments, the USP1 protein comprises the amino acid sequence of SEQ ID NO:1.

In another aspect, the present disclosure relates to a method of inhibiting a USP1 protein comprising contacting a USP1 protein with a Compound of the Disclosure, or a pharmaceutically acceptable salt or solvate thereof, or a pharmaceutical composition comprising a Compound of the Disclosure.

In some embodiments, the contacting occurs in vitro.

In some embodiments, the contacting occurs in vivo.

In some embodiments, the USP1 protein comprises the amino acid sequence

(SEQ ID NO: 1)
MPGVIPSESNGLSRGSPSKKNRLSLKFFQKKETKRALDFTDSQENEEKA
SEYRASEIDQVVPAAQSSPINCEKRENLLPFVGLNNLGNTCYLNSILQV
LYFCPGFKSGVKHLFNIISRKKEALKDEANQKDKGNCKEDSLASYELIC
SLQSLIISVEQLQASFLLNPEKYTDELATQPRRLLNTLRELNPMYEGYL
QHDAQEVLQCILGNIQETCQLLKKEEVKNVAELPTKVEEIPHPKEEMNG
INSIEMDSMRHSEDFKEKLPKGNGKRKSDTEFGNMKKKVKLSKEHQSLE
ENQRQTRSKRKATSDTLESPPKIIPKYISENESPRPSQKKSRVKINWLK
SATKQPSILSKFCSLGKITTNQGVKGQSKENECDPEEDLGKCESDNTTN
GCGLESPGNTVTPVNVNEVKPINKGEEQIGFELVEKLFQGQLVLRTRCL
ECESLTERREDFQDISVPVQEDELSKVEESSEISPEPKTEMKTLRWAIS
QFASVERIVGEDKYFCENCHHYTEAERSLLFDKMPEVITIHLKCFAASG
LEFDCYGGGLSKINTPLLTPLKLSLEEWSTKPTNDSYGLFAVVMHSGIT
ISSGHYTASVKVTDLNSLELDKGNFVVDQMCEIGKPEPLNEEEARGVVE
NYNDEEVSIRVGGNTQPSKVLNKKNVEAIGLLGGQKSKADYELYNKASN
PDKVASTAFAENRNSETSDTTGTHESDRNKESSDQTGINISGFENKISY
VVQSLKEYEGKWLLFDDSEVKVTEEKDFLNSLSPSTSPTSTPYLLFYKK
L.

### DETAILED DESCRIPTION OF THE DISCLOSURE

One aspect of the present disclosure is based on the use of Compounds of the Disclosure as inhibitors of a ubiquitin-specific-processing protease 1 (USP1) protein. In view of this property, the Compounds of the Disclosure are useful for inhibiting a USP1 protein and for treating diseases, disorders, or conditions, e.g., cancer, that are responsive to inhibition of a USP1 protein.

In some embodiments, certain Compounds of the Disclosure exhibit improved solubility, e.g., as measured by an ADME solubility assay as disclosed herein.

In some embodiments, certain Compounds of the Disclosure exhibit improved metabolic stability, e.g., as measured by liver microsome metabolic stability assays as disclosed herein.

In other embodiments, certain Compounds of the Disclosure exhibit improved duration of action and oral exposure in vivo.

In one embodiment, Compounds of the Disclosure are compounds having Formula I:

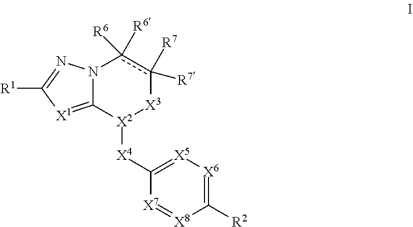

and the pharmaceutically acceptable salts or solvates, e.g., hydrates, thereof, wherein:

X¹ is selected from N and CR⁵;

X² is selected from N and CH;

X³ is selected from NR³, O, and CR³R^{3′};

X⁴ is selected from NR⁴, O, and CR⁴R^{4′};

each of X⁵, X⁶, X⁷, and X⁸ are independently selected from N and CR⁸;

R¹ is selected from optionally substituted C₆ aryl and optionally substituted 5- or 6-membered nitrogen-containing heteroaryl;

R² is selected from hydrogen, halo, alkoxy, optionally substituted (C₆-C₁₄) aryl, optionally substituted (C₆-C₁₄) ar-(C₁-C₂) alkyl, optionally substituted nitrogen-containing heteroaryl, optionally substituted, nitrogen-containing heteroar-(C₁-C₂) alkyl, optionally substituted (C₃-C₈) cycloalkyl, optionally substituted ((C₃-C₈) cycloalkyl)-(C₁-C₂) alkyl, optionally substituted heterocyclo, optionally substituted heterocyclo-(C₁-C₂) alkyl, optionally substituted (C₆-C₁₄) aryloxy, optionally substituted (C₆-C₁₄) ar-(C₁-C₂) alkyloxy, optionally substituted nitrogen-containing heteroaryloxy, optionally substituted nitrogen-containing heteroar-(C₁-C₂) alkyloxy, optionally substituted (C₃-C₈) cycloalkyloxy, optionally substituted ((C₃-C₈) cycloalkyl)-(C₁-C₂) alkyloxy, optionally substituted heterocyclooxy, optionally substituted heterocyclo-(C₁-C₂) alkyloxy;

each of R³ and R^{3′} is independently selected from hydrogen, halo, cyano, amino, hydroxy, alkoxy, optionally substituted alkyl, optionally substituted alkenyl, and optionally substituted alkynyl, and optionally substituted cycloalkyl; or

R³ and R^{3′} are taken together with the atom to which they are attached to form a carbonyl or an optionally substituted cycloalkyl;

each of R⁴ and R^{4′} is independently selected from hydrogen, halo, cyano, amino, optionally substituted alkyl, optionally substituted alkenyl, and optionally substituted alkynyl, and optionally substituted cycloalkyl;

R⁵ is selected from hydrogen, halo, cyano, amino, alkylamino, dialkylamino, hydroxy, alkoxy, optionally substituted alkyl, optionally substituted alkenyl, and optionally substituted alkynyl, and optionally substituted cycloalkyl;

each of R⁶ and R^{6′}, if present, is independently selected from hydrogen, halo, cyano, amino, alkylamino, dialkylamino, hydroxy, alkoxy, optionally substituted alkyl, optionally substituted alkenyl, and optionally substituted alkynyl, and optionally substituted cycloalkyl, and alkylcarbonylamino;

each of R⁷ and R^{7′}, if present, is independently selected from hydrogen, halo, cyano, amino, alkylamino, dialkylamino, hydroxy, alkoxy, optionally substituted alkyl, optionally substituted alkenyl, and optionally substituted alkynyl, optionally substituted cycloalkyl, and alkylcarbonylamino; or

R⁷ and R^{7′} are taken together with the atom to which they are attached to form a carbonyl or an optionally substituted cycloalkyl; and

R⁸ is selected from hydrogen, halo, cyano, amino, alkylamino, dialkylamino, hydroxy, and alkoxy; or

R⁸ and R⁴ or R^{4′} are taken together with the atoms to which they are attached to form an optionally substituted cycloalkyl.

In some embodiments, each of R³ and R^{3′} is independently selected from hydrogen, halo, cyano, amino, hydroxy, (C₁-C₄) alkoxy, optionally substituted (C₁-C₄) alkyl, optionally substituted (C₂-C₄) alkenyl, and optionally substituted (C₂-C₄) alkynyl, and optionally substituted (C₃-C₈) cycloalkyl; or

R³ and R^{3′} are taken together with the atom to which they are attached to form a carbonyl or an optionally substituted (C₃-C₈) cycloalkyl.

In some embodiments, each of R⁴ and R^{4′} is independently selected from hydrogen, halo, cyano, amino, optionally substituted (C₁-C₄) alkyl, optionally substituted (C₂-C₄) alkenyl, and optionally substituted (C₂-C₄) alkynyl, and optionally substituted (C₃-C₈) cycloalkyl.

In some embodiments, R⁵ is selected from hydrogen, halo, cyano, amino, (C₁-C₄) alkylamino, di-(C₁-C₄) alkylamino, hydroxy, (C₁-C₄) alkoxy, optionally substituted (C₁-C₄) alkyl, optionally substituted (C₂-C₄) alkenyl, and optionally substituted (C₂-C₄) alkynyl, and optionally substituted (C₃-C₈) cycloalkyl.

In some embodiments, each of R⁶ and R^{6′}, if present, is independently selected from hydrogen, halo, cyano, amino, (C₁-C₄) alkylamino, di-(C₁-C₄) alkylamino, hydroxy, (C₁-C₄) alkoxy, optionally substituted (C₁-C₄) alkyl, optionally substituted (C₂-C₄) alkenyl, and optionally substituted (C₂-C₄) alkynyl, and optionally substituted (C₃-C₈) cycloalkyl, and (C₁-C₄) alkylcarbonylamino.

In some embodiments, each of R⁷ and R^{7′}, if present, is independently selected from hydrogen, halo, cyano, amino, (C₁-C₄) alkylamino, di-(C₁-C₄) alkylamino, hydroxy, (C₁-C₄) alkoxy, optionally substituted (C₂-C₄) alkyl, optionally substituted (C₂-C₄) alkenyl, and optionally substituted (C₂-C₄) alkynyl, optionally substituted (C₃-C₈) cycloalkyl, and (C₁-C₄) alkylcarbonylamino; or

R⁷ and R^{7′} are taken together with the atom to which they are attached to form a carbonyl or an optionally substituted (C₃-C₈) cycloalkyl.

In some embodiments, R⁸ is selected from hydrogen, halo, cyano, amino, (C₁-C₄) alkylamino, di-(C₁-C₄) alkylamino, hydroxy, and (C₁-C₄) alkoxy; or

R⁸ and R⁴ or R^{4′} are taken together with the atoms to which they are attached to form an optionally substituted (C₃-C₈) cycloalkyl.

In one embodiment, a Compound of the Disclosure is a compound having Formula I. wherein

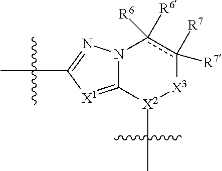

is independently selected from

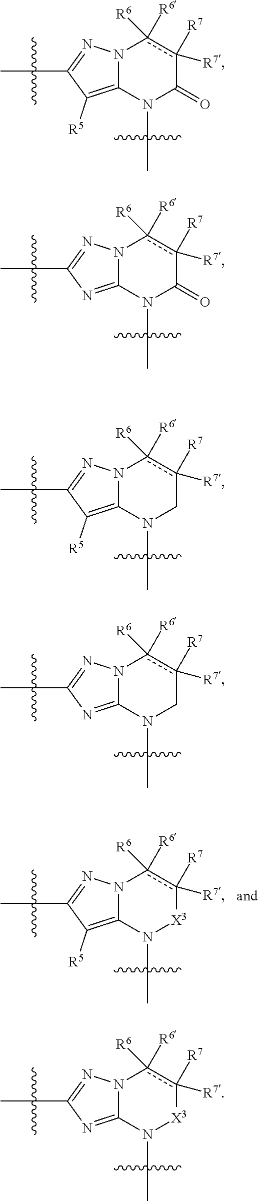

In another embodiment,

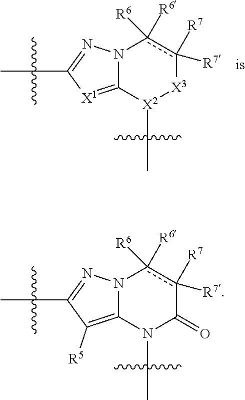

In another embodiment,

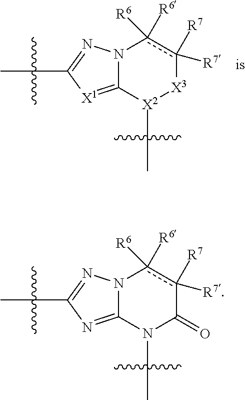

In another embodiment,

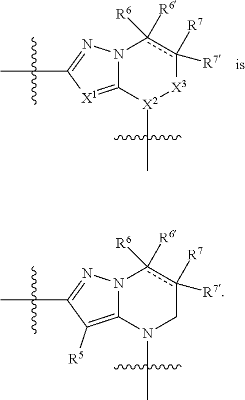

In another embodiment,

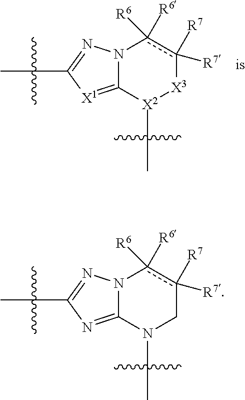

In another embodiment,

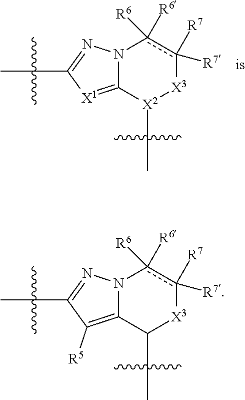

In another embodiment,

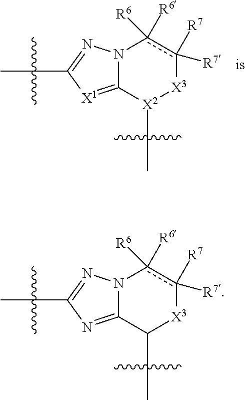

In one embodiment, a Compound of the Disclosure is a compound having Formula I, wherein

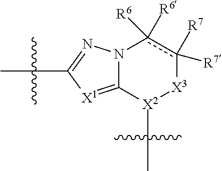

is independently selected from

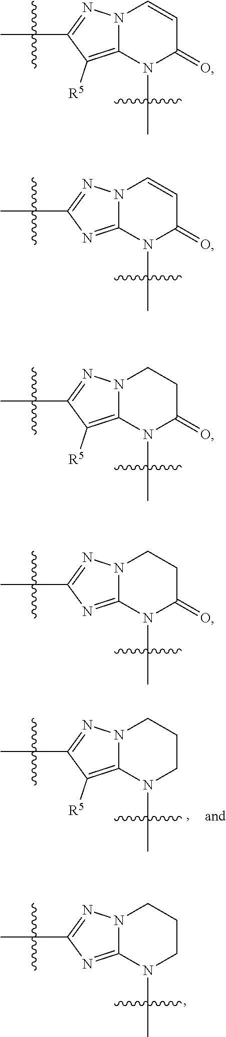

In another embodiment,

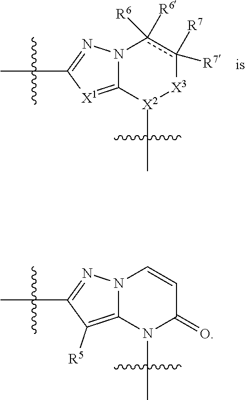

In another embodiment,

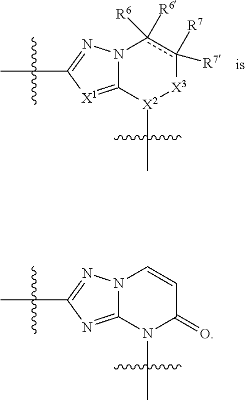

In another embodiment,

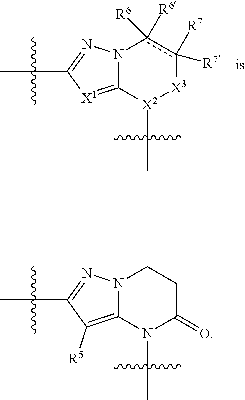

In another embodiment,

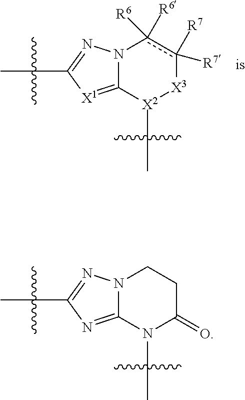

In another embodiment,

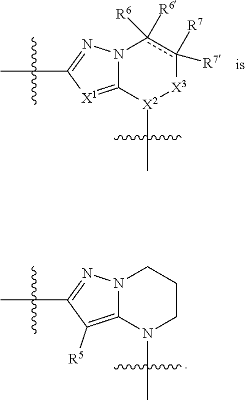

In another embodiment,

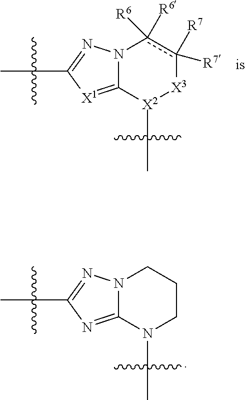

In one embodiment, a Compound of the Disclosure is a compound having Formula I, wherein X¹ is N. In another embodiment, X⁴ is CR⁴R^{4′}, X² is CH, X¹ is N, and X³ is CR³R^{3′}. In another embodiment, X⁴ is CR⁴R^{4′}, X² is CH, X¹ is N, and X³ is NR³.

In one embodiment, a Compound of the Disclosure is a compound having Formula I, wherein X¹ is CR⁵. In another embodiment, X⁴ is CR⁴R^{4′}, X² is CH, X¹ is CR⁵, and X³ is CR³R^{3′}. In another embodiment, X⁴ is CR⁴R^{4′}, X² is CH, X¹ is CR⁵, and X³ is NR³.

In one embodiment, a Compound of the Disclosure is a compound having Formula I, wherein X² is CH.

In one embodiment, a Compound of the Disclosure is a compound having Formula I, wherein X² is N.

In one embodiment, a Compound of the Disclosure is a compound having Formula I, wherein X³ is NR³.

In one embodiment, a Compound of the Disclosure is a compound having Formula I, wherein X³ is O.

In one embodiment, a Compound of the Disclosure is a compound having Formula I, wherein X³ is CR³R^{3′}.

In one embodiment, a Compound of the Disclosure is a compound having Formula I, wherein X⁴ is CR⁴R^{4′}. In another embodiment, X⁴ is CR⁴R^{4′} and X² is N. In another embodiment, X⁴ is CR⁴R^{4′}, X² is N, and X¹ is CR⁵. In another embodiment, X⁴ is CR⁴R^{4′}, X² is N, and X¹ is N. In another embodiment, X⁴ is CR⁴R^{4′}, X² is N, X¹ is N, and X³ is CR³R^{3′}. In another embodiment, X⁴ is CR⁴R^{4′}, X² is N, X¹ is CR⁵, and X³ is CR³R^{3′}.

In one embodiment, a Compound of the Disclosure is a compound having Formula I, wherein X⁴ is NR⁴. In another embodiment, X⁴ is NR⁴ and X² is CH. In another embodiment, X⁴ is NR⁴, X² is CH, and X¹ is N. In another embodiment, X⁴ is NR⁴, X² is CH, and X¹ is CR⁵. In another embodiment, X⁴ is NR⁴, X² is CH, X¹ is N, and X³ is CR³R^{3′}. In another embodiment, X⁴ is NR⁴, X² is CH, X¹ is CR⁵, and X³ is CR³R^{3′}.

In one embodiment, a Compound of the Disclosure is a compound having Formula I, wherein X⁴ is O. In another embodiment, X⁴ is O, X² is CH, X¹ is N, and X³ is CR³R^{3′}. In another embodiment, X⁴ is O, X² is CH, X¹ is CR⁵, and X³ is CR³R^{3′}. In another embodiment, X⁴ is O, X² is CH, X¹ is N, and X³ is NR³. In another embodiment, X⁴ is O, X² is CH, X¹ is CR⁵, and X³ is NR³.

In one embodiment, a Compound of the Disclosure is a compound having Formula I, wherein one of X⁵, X⁶, X⁷, and X⁸ is N. In another embodiment, X⁵ is N. In another embodiment, X⁶ is N. In another embodiment, X⁷ is N. In another embodiment, X⁸ is N.

In one embodiment, a Compound of the Disclosure is a compound having Formula I, wherein two of X⁵, X⁶, X⁷, and X⁸ is N. In another embodiment, X⁵ and X⁶ are N. In another embodiment, X⁵ and X⁷ are N. In another embodiment, X⁶ and X⁸ are N. In another embodiment, X⁶ and X⁷ are N.

In one embodiment, a Compound of the Disclosure is a compound having Formula I, wherein X⁶ is CR⁸.

In one embodiment, a Compound of the Disclosure is a compound having Formula I, wherein the optional substituents on R¹ are independently selected from hydrogen, halo, nitro, cyano, hydroxy, amino, alkylamino, dialkylamino, haloalkyl, hydroxyalkyl, alkoxy, haloalkoxy, aryloxy, heteroaryloxy, aralkyl aralkyloxy, alkylthio, carboxamido, sulfonamido, alkylcarbonyl, arylcarbonyl, alkyl sulfonyl, aryl sulfonyl, carboxy, carboxyalkyl, alkyl, optionally substituted cycloalkyl, alkenyl, alkynyl, optionally substituted aryl, optionally substituted heteroaryl, optionally substituted heterocyclo, alkoxyalkyl, (amino)alkyl, hydroxyalkylamino, (alkylamino)alkyl, (dialkylamino)alkyl, (cyano)alkyl, (carboxamido)alkyl, mercaptoalkyl, (heterocyclo)alkyl, (cycloalkylamino)alkyl, (C₁₋₄ haloalkoxy)alkyl, and (heteroaryl)alkyl.

In another embodiment, a Compound of the Disclosure is a compound having Formula I, wherein two of the optional substituents on R¹ are taken together with the carbon or nitrogen atoms to which they are attached to form an optionally substituted cycloalkyl, optionally substituted heterocyclo, optionally substituted aryl, or optionally substituted heteroaryl group.

In one embodiment, a Compound of the Disclosure is a compound having Formula I, wherein the optional substituents on R¹ are independently selected from hydrogen, halo, nitro, cyano, hydroxy, and amino.

In one embodiment, a Compound of the Disclosure is a compound having Formula I, wherein the optional substituents on R¹ are independently selected from (C₁₋₄) alkylamino, di-(C₁₋₄) alkylamino, halo-(C₁₋₄) alkyl, hydroxyl-(C₁₋₄) alkyl, (C₁₋₄) alkoxy, halo-(C₁₋₄) alkoxy, (C₆₋₁₀) aryloxy, (C₃₋₆) heteroaryloxy, ar-(C₁₋₄) alkyl, ar-(C₁₋₄) alkyloxy, (C₁₋₄) alkylthio, carboxamido, sulfonamido, (C₁₋₄) alkylcarbonyl, (C₆₋₁₀) arylcarbonyl, (C₁₋₄) alkylsulfonyl, (C₆₋₁₀) arylsulfonyl, carboxy, carboxy-(C₁₋₄) alkyl, (C₁₋₄) alkyl, optionally substituted (C₃₋₈) cycloalkyl, (C₂₋₄) alkenyl, (C₂₋₄) alkynyl, optionally substituted (C₆₋₁₀) aryl, optionally substituted (C₃-C₆) heteroaryl, optionally substituted (C₃₋₈) heterocyclo, alkoxy-(C₁₋₄) alkyl, (amino)-(C₁₋₄) alkyl, hydroxyl-(C₁₋₄) alkylamino, ((C₁₋₄) alkylamino)-(C₁₋₄) alkyl, (di-(C₁₋₄) alkylamino)-(C₁₋₄) alkyl, (cyano)-(C₁₋₄) alkyl, (carboxamido)-(C₁₋₄) alkyl, mercapto-(C₁₋₄) alkyl, ((C₃-C₈) heterocyclo)-(C₁₋₄) alkyl, ((C₃₋₈) cycloalkylamino)-(C₁₋₄) alkyl, (C₁₋₄ haloalkoxy)-(C₁₋₄) alkyl, and ((C₃-C₆) heteroaryl)-(C₁₋₄) alkyl.

In one embodiment, a Compound of the Disclosure is a compound having Formula I, wherein R¹ is an optionally substituted 5- or 6-membered nitrogen-containing heteroaryl, wherein nitrogen is the only heteroatom.

In one embodiment, a Compound of the Disclosure is a compound having Formula I, wherein R¹ is an optionally substituted pyrimidin-5-yl. In another embodiment, the pyrimidin-5-yl is optionally substituted at the 4-position. In another embodiment, the pyrimidin-5-yl is optionally substituted at the 6-position. In another embodiment, the pyrimidin-5-yl is optionally disubstituted at the 4- and 6-positions. In another embodiment, the pyrimidin-5-yl is optionally trisubstituted at the 2-, 4-, and 6-positions.

In one embodiment, a Compound of the Disclosure is a compound having Formula I, wherein R¹ is an optionally substituted pyrazol-5-yl. In another embodiment, the pyrazol-5-yl is optionally substituted at the 1-position. In another embodiment, the pyrazol-5-yl is optionally substituted at the 4-position. In another embodiment, the pyrazol-5-yl is optionally disubstituted at the 1- and 4-positions.

In one embodiment, a Compound of the Disclosure is a compound having Formula I, wherein R¹ is an optionally substituted pyrid-3-yl. In another embodiment, the pyrid-3-yl is optionally substituted at the 2-position. In another embodiment, the pyrid-3-yl is optionally substituted at the 4-position. In another embodiment, the pyrid-3-yl is optionally disubstituted at the 2- and 4-positions.

In one embodiment, a Compound of the Disclosure is a compound having Formula I, wherein R¹ is and optionally substituted pyrid-4-yl. In another embodiment, the pyrid-4-yl is optionally substituted at the 3-position. In another embodiment, the pyrid-4-yl is optionally substituted at the 5-position. In another embodiment, the pyrid-4-yl is optionally disubstituted at the 3- and 5-positions.

In one embodiment, a Compound of the Disclosure is a compound having Formula I, wherein R¹ is an optionally substituted phenyl. In another embodiment, the phenyl is optionally substituted at the 2-position. In another embodiment, the phenyl is optionally substituted at the 6-position. In another embodiment, the phenyl is optionally disubstituted at the 2- and 6-positions. In another embodiment, the phenyl is optionally disubstituted at the 2- and 3-positions.

In one embodiment, a Compound of the Disclosure is a compound having Formula I, wherein R¹ is selected from optionally substituted C₆ aryl and optionally substituted 5- or 6-membered nitrogen-containing heteroaryl.

In one embodiment, a Compound of the Disclosure is a compound having Formula I, wherein R¹ is substituted and the substituents are independently selected from hydrogen, cyano, amino, methylamino, dimethylamino, methoxy, ethoxy, isopropoxy, tert-butoxy, difluoromethoxy, trifluoromethoxy, cyclopropoxy, cyclobutoxy, methyl, ethyl, propyl, isopropyl, butyl, isobutyl, difluoromethyl, trifluoromethyl, cyclopropyl, cyclobutyl, isopropyl, tert-butyl, chloro, fluoro, 1-fluoropropan-2-yl, (S)-1-fluoropropan-2-yl, (R)-1-fluoropropan-2-yl, hydroxyethyl, 1-methoxy-2-methylpropan-2-yl, 1-methoxypropan-2-yl, (5)-1-methoxypropan-2-yl, (R)-1-methoxypropan-2-yl, 1-(methoxymethyl)cyclopropyl, 1-hydroxypropan-2-yl, oxetan-3-yl, tetrahydrofuran-3-yl, 1-methylcyclopropyl, deuteromethyl, deuteroethyl, deuteroisopropyl, deuteromethoxy, and deuteroethoxy.

In one embodiment, a Compound of the Disclosure is a compound having Formula I, wherein R¹ is independently selected from:

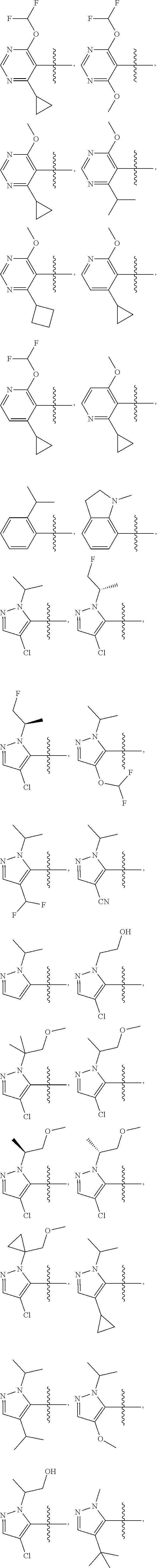

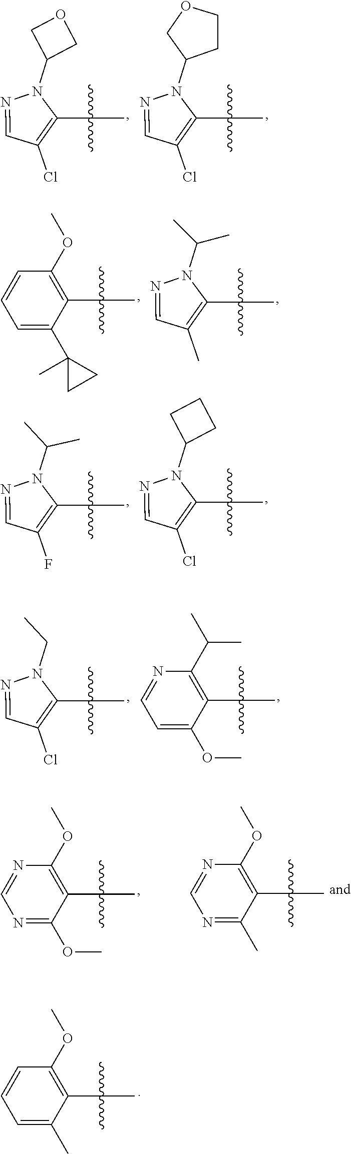

In one embodiment, a Compound of the Disclosure is a compound having Formula I, wherein the optional substituents on R² are independently selected from hydrogen, halo, nitro, cyano, hydroxy, amino, alkylamino, dialkylamino, haloalkyl, hydroxyalkyl, alkoxy, haloalkoxy, aryloxy, heteroaryloxy, optionally substituted cycloalkyloxy, aralkyl aralkyloxy, alkylthio, carboxamido, sulfonamido, alkylcarbonyl, arylcarbonyl, alkylsulfonyl, arylsulfonyl, carboxy, carboxyalkyl, alkyl, optionally substituted cycloalkyl, alkenyl, alkynyl, optionally substituted aryl, optionally substituted heteroaryl, optionally substituted heterocyclo, alkoxyalkyl, (amino)alkyl, hydroxyalkylamino, (alkylamino)alkyl, (dialkylamino)alkyl, (cyano)alkyl, (carboxamido)alkyl, mercaptoalkyl, (heterocyclo)alkyl, (cycloalkylamino)alkyl, (C₁₋₄ haloalkoxy)alkyl, (heteroaryl)alkyl, and sulfonamide.

In one embodiment, a Compound of the Disclosure is a compound having Formula I, wherein two of the optional substituents on R² are taken together with the carbon or nitrogen atoms to which they are attached to form an optionally substituted cycloalkyl, optionally substituted heterocyclo, optionally substituted aryl, or optionally substituted heteroaryl group.

In one embodiment, a Compound of the Disclosure is a compound having Formula I, wherein the optional substituents on R² are independently selected from hydrogen, halo, nitro, cyano, hydroxy, and amino.

In one embodiment, a Compound of the Disclosure is a compound having Formula I, wherein the optional substituents on R² are independently selected from (C₁₋₄) alkylamino, di-(C₁₋₄) alkylamino, halo-(C₁₋₄) alkyl, hydroxyl-(C₁₋₄) alkyl, (C₁₋₄) alkoxy, halo-(C₁₋₄) alkoxy, (C₆₋₁₀) aryloxy, (C₃₋₆) heteroaryloxy, optionally substituted (C₃₋₈) cycloalkyloxy, (C₆₋₁₀) ar-(C₁₋₄) alkyl, (C₆₋₁₀) ar-(C₁₋₄) alkyloxy, (C₁₋₄) alkylthio, carboxamido, (C₁₋₄) alkylcarbonyl, (C₆₋₁₀) arylcarbonyl, (C₁₋₄) alkyl sulfonyl, (C₆₋₁₀) arylsulfonyl, carboxy, carboxy-(C₁₋₄) alkyl, (C₁₋₄) alkyl, optionally substituted (C₃₋₈) cycloalkyl, (C₂₋₄) alkenyl, (C₂₋₄) alkynyl, optionally substituted (C₆₋₁₀) aryl, optionally substituted (C₃-C₆) heteroaryl, optionally substituted (C₃₋₈) heterocyclo, alkoxy-(C₁₋₄) alkyl, (amino)-(C₁₋₄) alkyl, hydroxyl-(C₁₋₄) alkylamino, ((C₁₋₄) alkylamino)-(C₁₋₄) alkyl, (di-(C₁₋₄) alkylamino)-(C₁₋₄) alkyl, (cyano)-(C₁₋₄) alkyl, (carboxamido)-(C₁₋₄) alkyl, mercapto-(C₁₋₄) alkyl, ((C₃₋₈) heterocyclo)-(C₁₋₄) alkyl, ((C₃₋₈) cycloalkylamino)-(C₁₋₄) alkyl, (C₁₋₄ haloalkoxy)-(C₁₋₄) alkyl, ((C₃-C₆) heteroaryl)-(C₁₋₄) alkyl, and sulfonamido.

In one embodiment, a Compound of the Disclosure is a compound having Formula I, wherein R² is an optionally substituted 5- or 6-membered nitrogen-containing heteroaryl. In another embodiment, R² is an optionally substituted 5- or 6-membered nitrogen-containing heteroaryl, wherein nitrogen is the only heteroatom.

In one embodiment, a Compound of the Disclosure is a compound having Formula I, wherein R² is an optionally substituted imidazolyl.

In one embodiment, a Compound of the Disclosure is a compound having Formula I, wherein R² is an optionally substituted pyridyl.

In one embodiment, a Compound of the Disclosure is a compound having Formula I, wherein R² is an optionally substituted pyrazolyl.

In one embodiment, a Compound of the Disclosure is a compound having Formula I, wherein R² is an optionally substituted pyridazinyl.

In one embodiment, a Compound of the Disclosure is a compound having Formula I, wherein R² is an optionally substituted pyrimidinyl.

In one embodiment, a Compound of the Disclosure is a compound having Formula I, wherein R² is an optionally substituted triazinyl.

In one embodiment, a Compound of the Disclosure is a compound having Formula I, wherein R² is an optionally substituted pyrazinyl.

In one embodiment, a Compound of the Disclosure is a compound having Formula I, wherein R² is an optionally substituted triazolyl.

In one embodiment, a Compound of the Disclosure is a compound having Formula I, wherein R² is substituted and the substituents are independently selected from hydrogen, cyano, nitro, fluoro, chloro, bromo, methyl, ethyl, propyl, isopropyl, butyl, isobutyl, tert-butyl, trifluoromethyl, methoxy, ethoxy, isopropoxy, tert-butoxy, difluoromethoxy, trifluoromethoxy, 1-fluoropropan-2-yl, 2-fluoroethyl, amino, methylamino, ethylamino, dimethylamino, 2,2-difluoroethoxy, cyclopropoxy, morpholino, methoxymethyl, N,N-dimethylsulfonamido, cyclopropyl, methylaminomethyl, deuteromethyl, deuteroethyl, deuteroisopropyl, deuteromethoxy, and deuteroethoxy.

In one embodiment, a Compound of the Disclosure is a compound having Formula I, wherein R² is independently selected from:

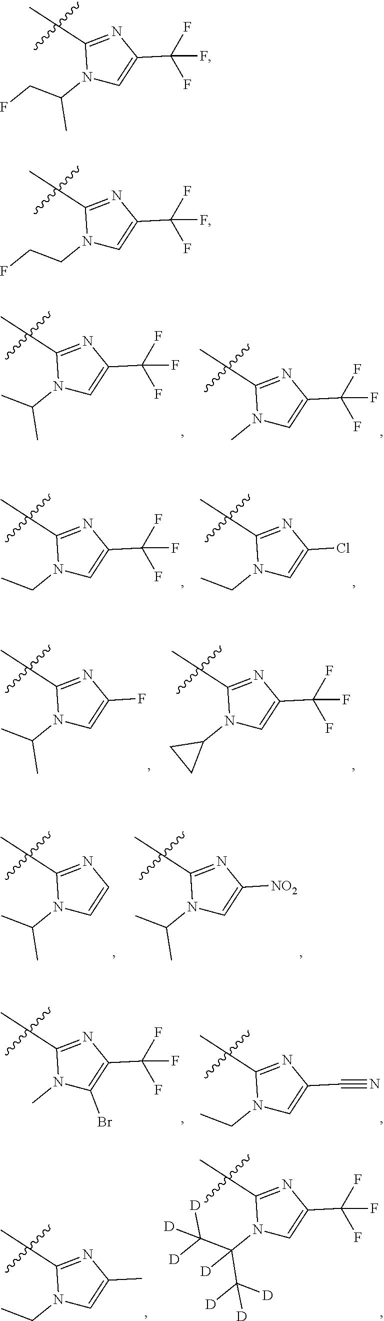

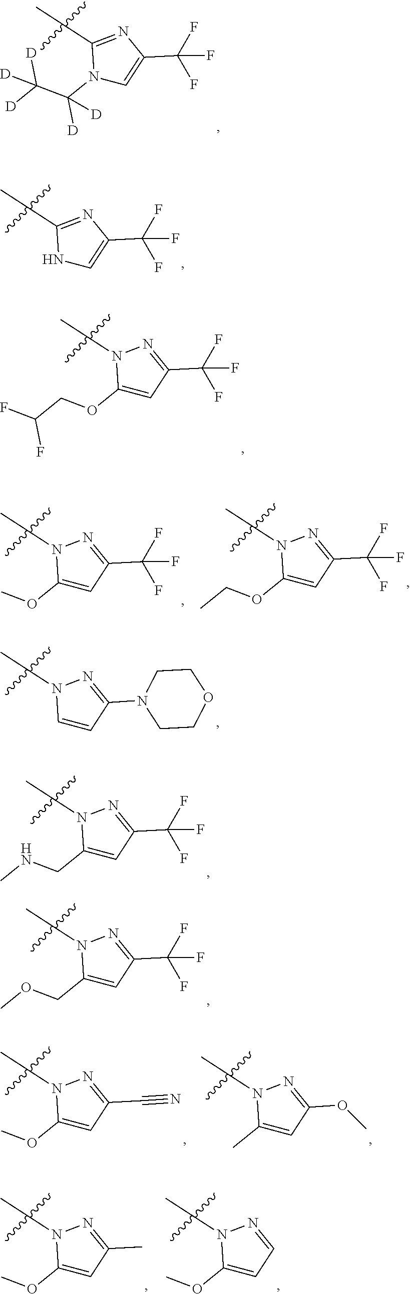

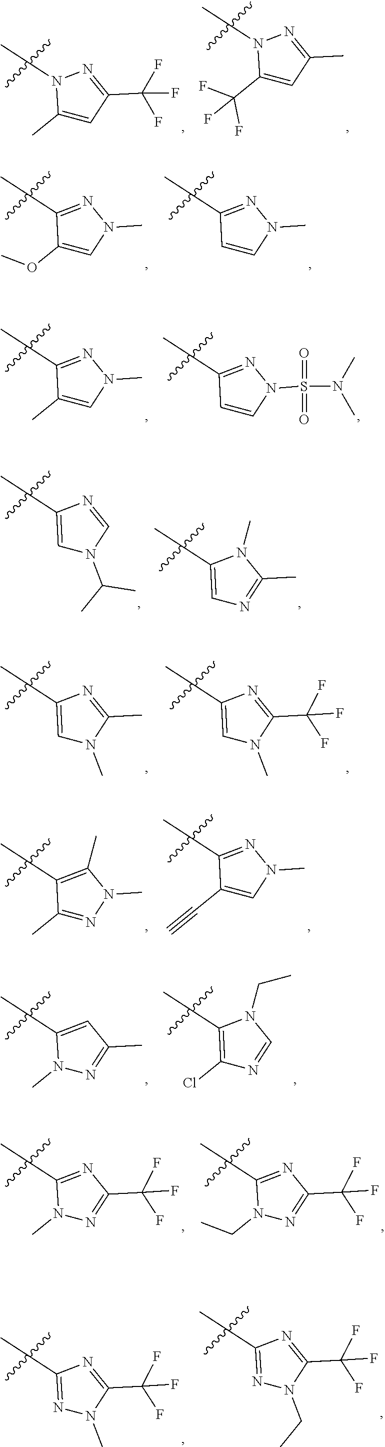

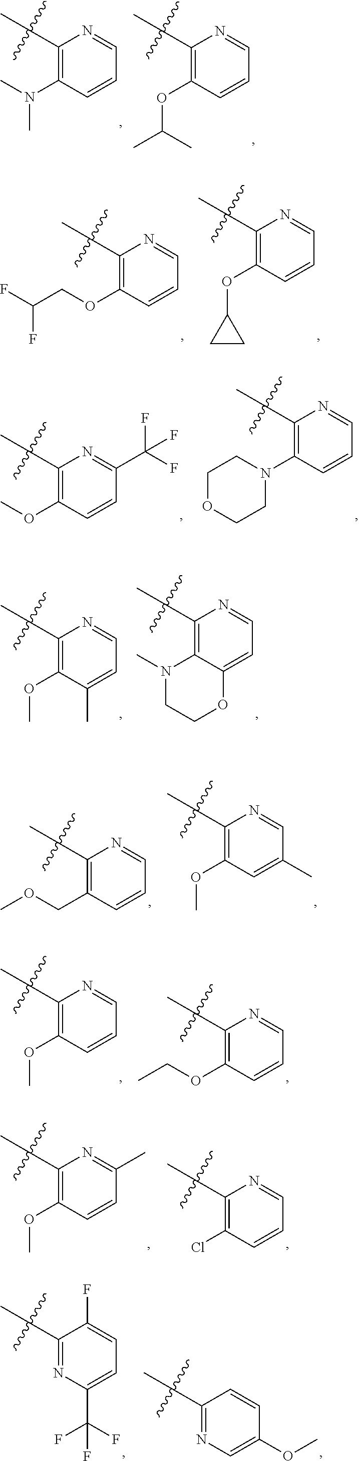

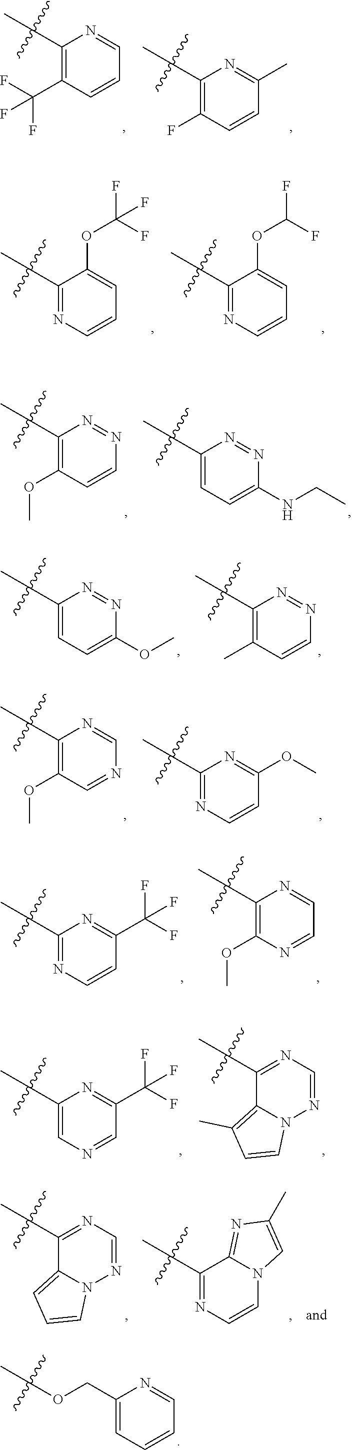

In one embodiment, a Compound of the Disclosure is a compound having Formula I, wherein

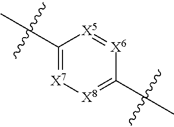

is independently selected from

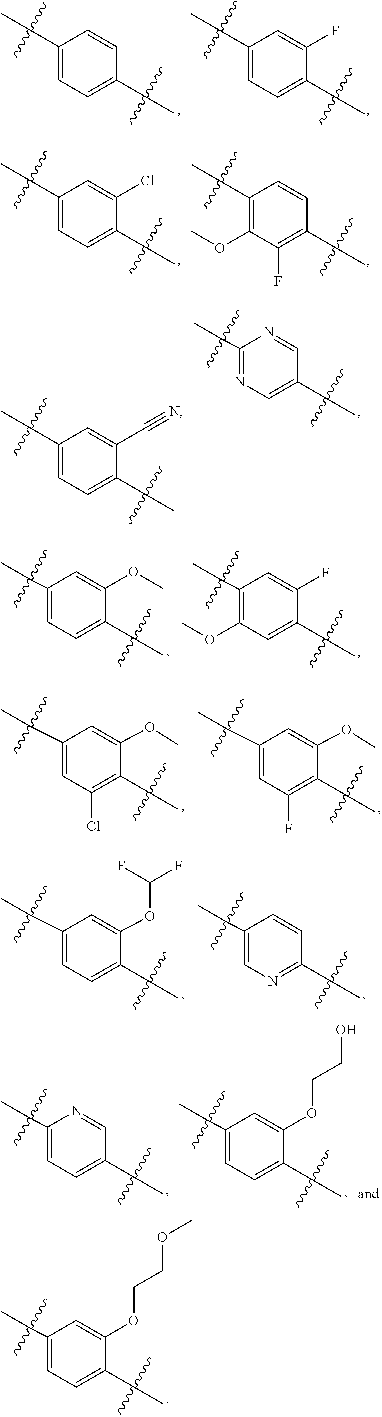

In one embodiment, a Compound of the Disclosure is a compound having Formula II, Formula III, Formula IV, Formula V, Formula VI, or Formula VII:

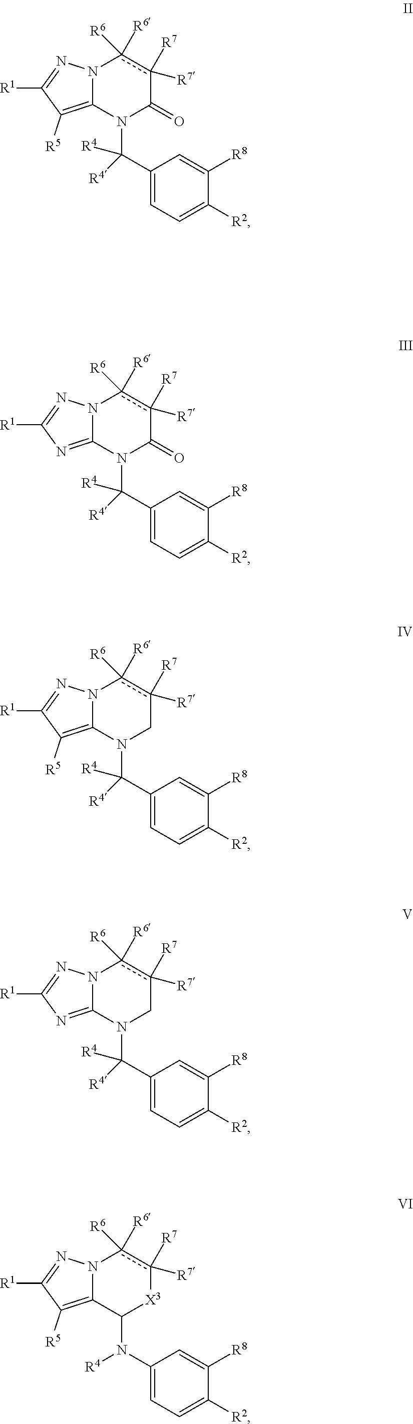

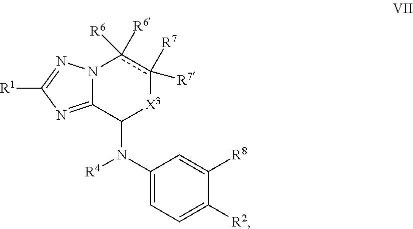

wherein:

R¹ is selected from

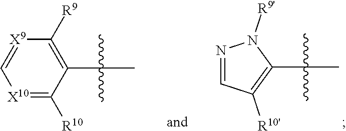

R² is selected from

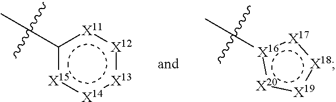

R⁴ and R^{4′} are independently selected from hydrogen, halo, and (C₁-C₃) alkyl;

R⁵ is selected from hydrogen, halo, cyano, amino, alkylamino, dialkylamino, and (C₁-C₃) alkyl;

X⁹ is selected from N and CH;

X¹⁰ is selected from N and CH;

X¹¹ is selected from N and CR″;

X¹² is selected from N and CR¹²;

X¹³ is selected from N and CR¹³;

X¹⁴ is selected from N and CR¹⁴;

X¹⁵ is selected from N and CR¹⁵;

X¹⁶ is selected from N and CR¹⁶;

X¹⁷ is selected from N and CR¹⁷;

X¹⁸ is selected from N and CR^{IB};

X¹⁹ is selected from N and CR¹⁹;

X²⁰ is selected from N and CR²⁰,

each of R⁹, R^{9′}, R¹⁰, R^{10′}, R¹¹, R¹², R¹³, R¹⁴, R¹⁵, R¹⁶, R¹⁷, R¹⁸, R¹⁹_{, R}²⁰ is independently selected from the group consisting of hydrogen, halo, nitro, cyano, hydroxy, amino, alkylamino, dialkylamino, haloalkyl, hydroxyalkyl, alkoxy, haloalkoxy, aryloxy, heteroaryloxy, aralkyl, aralkyloxy, alkylthio, carboxamido, sulfonamido, alkylcarbonyl, arylcarbonyl, alkyl sulfonyl, arylsulfonyl, carboxy, carboxyalkyl, alkyl, optionally substituted cycloalkyl, alkenyl, alkynyl, optionally substituted aryl, optionally substituted heteroaryl, optionally substituted heterocyclo, alkoxyalkyl, (amino)alkyl, hydroxyalkylamino, (alkylamino)alkyl, (dialkylamino)alkyl, (cyano)alkyl, (carboxamido)alkyl, mercaptoalkyl, (heterocyclo)alkyl, (cycloalkylamino)alkyl, (C₁₋₄ haloalkoxy)alkyl, (heteroaryl)alkyl, or sulfonamide; or

wherein two of R¹¹, R¹², R¹³, R¹⁴, and R¹⁵ or two of R¹⁶, R¹⁷, R¹⁸, R¹⁹, and R²⁰ are taken together with the atom to which they are attached to form an optionally substituted cycloalkyl, optionally substituted heterocyclo, optionally substituted heteroaryl, or optionally substituted aryl.

In another embodiment, each of R⁹, R^{9′}, R¹⁰, R^{10′}, R¹¹, R¹², R¹³, R¹⁴, R¹⁵, R¹⁶, R¹⁷, R¹⁸, R¹⁹, R²⁰ is independently selected from the group consisting of hydrogen, halo, nitro, cyano, hydroxy, and amino.

In another embodiment, each of R⁹, R^{9′}, R¹⁰, R^{10′}, R¹¹, R¹², R¹³, R¹⁴, R¹⁵, R¹⁶, R¹⁷, R¹⁸, R¹⁹, R²⁰ is independently selected from the group consisting of (C₁₋₄) alkylamino, di-(C₁₋₄) alkylamino, halo-(C₁₋₄) alkyl, hydroxyl-(C₁₋₄) alkyl, (C₁₋₄) alkoxy, halo-(C₁₋₄) alkoxy, (C₆₋₁₀) aryloxy, (C₃₋₆) heteroaryloxy, (C₆₋₁₀) aralkyl, aralkyloxy, alkylthio, carboxamido, alkylcarbonyl, (C₆₋₁₀) arylcarbonyl, alkyl sulfonyl, (C₆₋₁₀) aryl sulfonyl, carboxy, carboxy-(C₁₋₄) alkyl, (C₁₋₄) alkyl, optionally substituted (C₃₋₈) cycloalkyl, (C₂₋₄) alkenyl, (C₂₋₄) alkynyl, optionally substituted (C₆₋₁₀) aryl, optionally substituted (C₃-C₆) heteroaryl, optionally substituted (C₃₋₈) heterocyclo, alkoxy-(C₁₋₄) alkyl, (amino)-(C₁₋₄) alkyl, hydroxyl-(C₁₋₄) alkylamino, ((C₁₋₄) alkylamino)-(C₁₋₄) alkyl, (di-(C₁₋₄) alkylamino)-(C₁₋₄) alkyl, (cyano)-(C₁₋₄) alkyl, (carboxamido)-(C₁₋₄) alkyl, mercapto-(C₁₋₄) alkyl, ((C₃₋₈) heterocyclo)-(C₁₋₄) alkyl, ((C₃₋₈) cycloalkylamino)-(C₁₋₄) alkyl, (C₁₋₄ haloalkoxy)-(C₁₋₄) alkyl, ((C₃-C₆) heteroaryl)-(C₁₋₄) alkyl, and sulfonamido.

In another embodiment, a Compound of the Disclosure is a compound having Formula II:

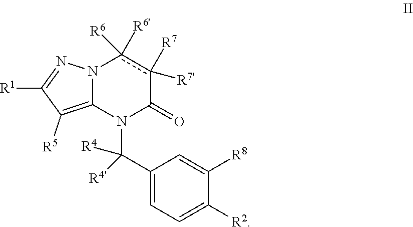

In another embodiment, a Compound of the Disclosure is a compound having Formula III:

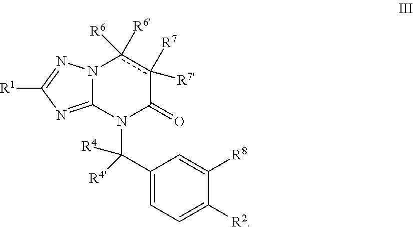

In another embodiment, a Compound of the Disclosure is a compound having Formula IV:

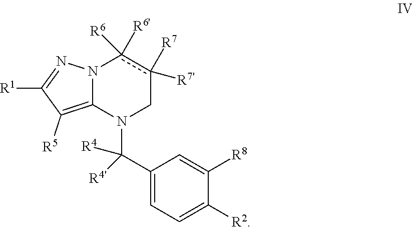

In another embodiment, a Compound of the Disclosure is a compound having Formula V:

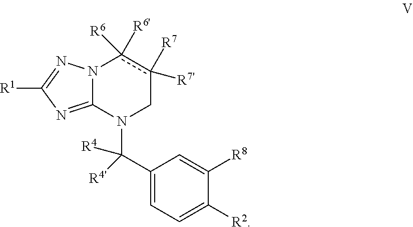

In another embodiment, a Compound of the Disclosure is a compound having Formula VI:

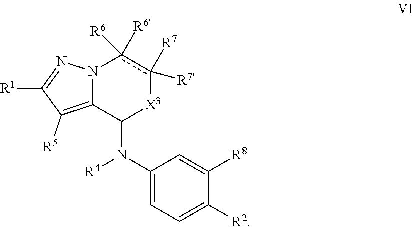

In another embodiment, a Compound of the Disclosure is a compound having Formula VII:

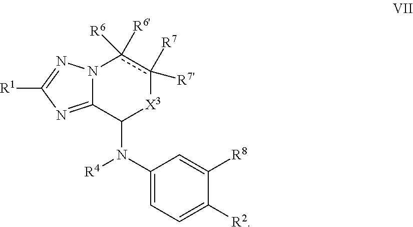

In another embodiment, a Compound of the Disclosure is a compound having Formula II, Formula III, Formula IV, Formula V, Formula VI, or Formula VII, wherein R¹ is

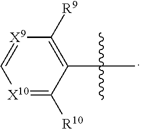

In another embodiment, a Compound of the Disclosure is a compound having Formula II, Formula III, Formula IV, Formula V, Formula VI, or Formula VII, wherein R¹ is

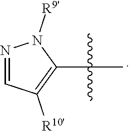

In another embodiment, a Compound of the Disclosure is a compound having Formula II, Formula III, Formula IV, Formula V, Formula VI, or Formula VII, wherein R² is

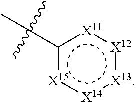

In another embodiment, a Compound of the Disclosure is a compound having Formula II, Formula III, Formula IV, Formula V, Formula VI, or Formula VII, wherein R² is

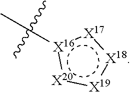

In one embodiment, a Compound of the Disclosure is a compound having Formula VIII, or Formula IX

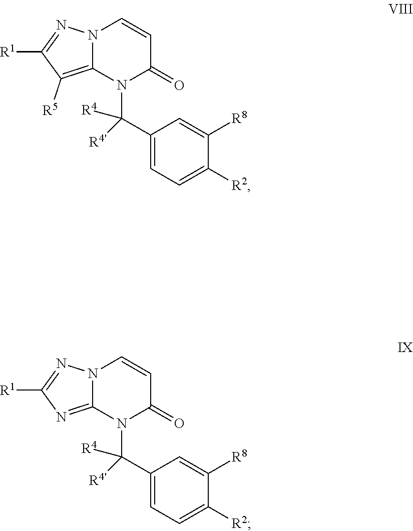

wherein R⁴ and R^{4′} are independently selected from hydrogen and methyl; and R⁸ is selected from hydrogen and fluoro.

In another embodiment, a Compound of the Disclosure is a compound having Formula VIII,

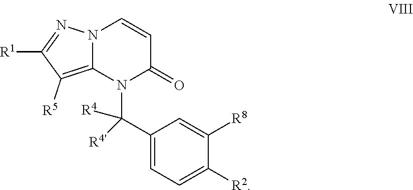

In another embodiment, a Compound of the Disclosure is a compound having Formula IX,

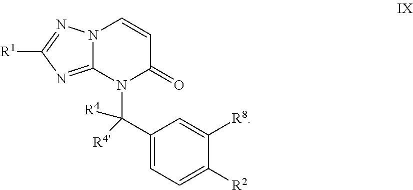

In one embodiment, a Compound of the Disclosure is a compound having Formula X, or Formula XI,

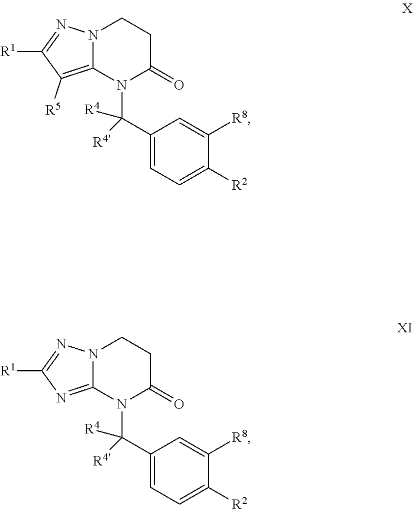

wherein R⁴ and R^{4′} are independently selected from hydrogen and methyl; and R⁸ is selected from hydrogen and fluoro.

In another embodiment, a Compound of the Disclosure is a compound having Formula X,

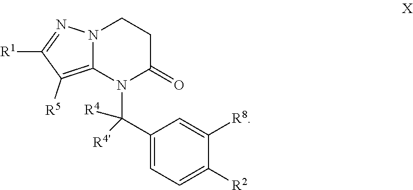

In another embodiment, a Compound of the Disclosure is a compound having Formula XI,

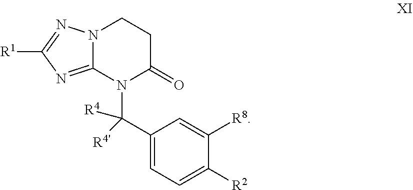

In one embodiment, a Compound of the Disclosure is a compound having Formula XII, or Formula XIII

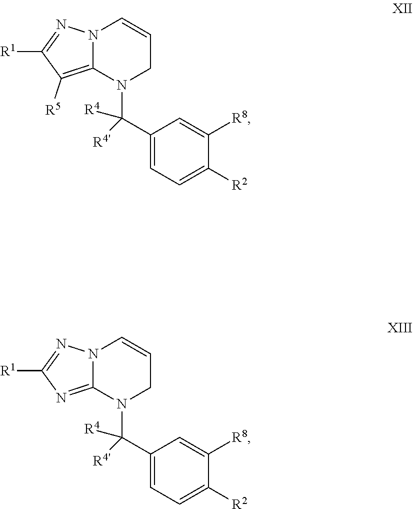

wherein R⁴ and R^{4′} are independently selected from hydrogen and methyl; and R⁸ is selected from hydrogen and fluoro.

In another embodiment, a Compound of the Disclosure is a compound having Formula XII,

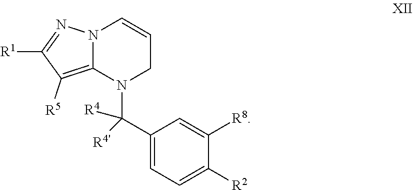

In another embodiment, a Compound of the Disclosure is a compound having Formula XIII,

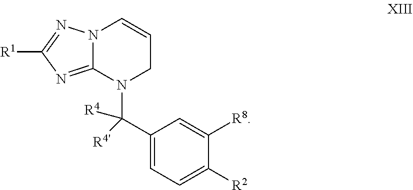

In one embodiment, a Compound of the Disclosure is a compound having Formula XIV, or Formula XV

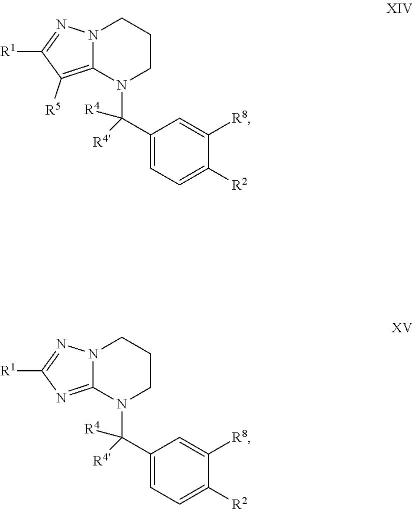

wherein R⁴ and R^{4′} are independently selected from hydrogen and methyl; and R⁸ is selected from hydrogen and fluoro.

In another embodiment, a Compound of the Disclosure is a compound having Formula XIV,

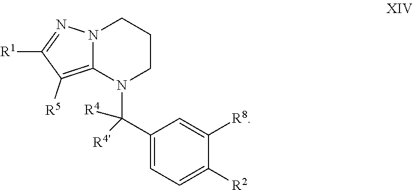

In another embodiment, a Compound of the Disclosure is a compound having Formula XV,

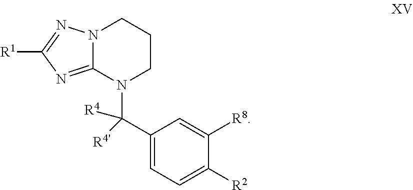

In one embodiment, a Compound of the Disclosure is a compound having Formula I, wherein R⁸ is selected from hydrogen and fluoro. In another embodiment, R⁸ is hydrogen. In another embodiment, R⁸ is fluoro.

In one embodiment, a Compound of the Disclosure is a compound having Formula I, wherein R⁸ is selected from cyano, chloro, methoxy, methoxyethoxy, hydroxyethoxy, and difluoromethoxy.

In one embodiment, Compounds of the Disclosure are compounds selected from the group consisting of:

2-(4-cyclopropyl-6-methoxypyrimidin-5-yl)-4-(4-(1-methyl-4-(trifluoromethyl)-1H-imidazol-2-yl)benzyl)-6,7-dihydropyrazolo[1,5-α]pyrimidin-5(4H)-one;

2-(4-cyclopropyl-6-methoxypyrimidin-5-yl)-4-(3-fluoro-4-(1-methyl-4-(trifluoromethyl)-1H-imidazol-2-yl)benzyl)-6,7-dihydropyrazolo[1,5-α]pyrimidin-5(4H)-one;

2-(4-cyclopropyl-6-methoxypyrimidin-5-yl)-4-(1-(4-(1-methyl-4-(trifluoromethyl)-1H-imidazol-2-yl)phenyl)ethyl)-6,7-dihydropyrazolo[1,5-α]pyrimidin-5(4H)-one;

2-(4-cyclopropyl-6-methoxypyrimidin-5-yl)-4-(4-(5-methyl-3-(trifluoromethyl)-1H-pyrazol-1-yl)benzyl)-6,7-dihydropyrazolo[1,5-α]pyrimidin-5(4H)-one;

2-(4,6-dimethoxypyrimidin-5-yl)-4-(4-(1-ethyl-4-(trifluoromethyl)-1H-imidazol-2-yl)benzyl)-6,7-dihydropyrazolo[1,5-α]pyrimidin-5(4H)-one;

2-(1-isopropyl-4-methyl-1H-pyrazol-5-yl)-4-(4-(1-methyl-4-(trifluoromethyl)-1H-imidazol-2-yl)benzyl)-6,7-dihydropyrazolo[1,5-α]pyrimidin-5(4H)-one;

2-(4-cyclopropyl-6-methoxypyrimidin-5-yl)-4-(3-fluoro-4-(5-methoxy-3-(trifluoromethyl)-1H-pyrazol-1-yl)benzyl)-6,7-dihydropyrazolo[1,5-α]pyrimidin-5(4H)-one;

1-(4-((2-(4-cyclopropyl-6-methoxypyrimidin-5-yl)-5-oxo-6,7-dihydropyrazolo[1,5-α]pyrimidin-4(5H)-yl)methyl)-2-fluorophenyl)-5-methoxy-1H-pyrazole-3-carbonitrile;

4-(4-(1-ethyl-4-(trifluoromethyl)-1H-imidazol-2-yl)benzyl)-2-(1-isopropyl-4-methyl-1H-pyrazol-5-yl)-6,7-dihydropyrazolo[1,5-α]pyrimidin-5(4H)-one;

2-(1-isopropyl-4-methyl-1H-pyrazol-5-yl)-4-(4-(5-methyl-3-(trifluoromethyl)-1H-pyrazol-1-yl)benzyl)-6,7-dihydropyrazolo[1,5-α]pyrimidin-5(4H)-one;

2-(1-isopropyl-4-methyl-1H-pyrazol-5-yl)-4-(4-(1-methyl-3-(trifluoromethyl)-1H-1,2,4-triazol-5-yl)benzyl)-6,7-dihydropyrazolo[1,5-α]pyrimidin-5(4H)-one;

4-(3-fluoro-4-(5-methoxy-3-(trifluoromethyl)-1H-pyrazol-1-yl)benzyl)-2-(1-isopropyl-4-methyl-1H-pyrazol-5-yl)-6,7-dihydropyrazolo[1,5-α]pyrimidin-5(4H)-one;

2-(2-isopropyl-4-methoxypyridin-3-yl)-4-(4-(1-methyl-4-(trifluoromethyl)-1H-imidazol-2-yl)benzyl)-6,7-dihydropyrazolo[1,5-α]pyrimidin-5(4H)-one;

2-(4-chloro-1-isopropyl-1H-pyrazol-5-yl)-4-(3-fluoro-4-(5-methoxy-3-(trifluoromethyl)-1H-pyrazol-1-yl)benzyl)-6,7-dihydropyrazolo[1,5-α]pyrimidin-5(4H)-one;

4-(4-(1-ethyl-5-(trifluoromethyl)-1H-1,2,4-triazol-3-yl)benzyl)-2-(1-isopropyl-4-methyl-1H-pyrazol-5-yl)-6,7-dihydropyrazolo[1,5-α]pyrimidin-5(4H)-one;

2-(4-chloro-1-isopropyl-1H-pyrazol-5-yl)-4-(3-fluoro-4-(1-methyl-4-(trifluoromethyl)-1H-imidazol-2-yl)benzyl)-6,7-dihydropyrazolo[1,5-α]pyrimidin-5(4H)-one;

2-(4-chloro-1-ethyl-1H-pyrazol-5-yl)-4-(3-fluoro-4-(1-methyl-4-(trifluoromethyl)-1H-imidazol-2-yl)benzyl)-6,7-dihydropyrazolo[1,5-α]pyrimidin-5(4H)-one;

2-(4-cyclopropyl-6-methoxypyrimidin-5-yl)-4-(3-fluoro-4-(1-isopropyl-4-(trifluoromethyl)-1H-imidazol-2-yl)benzyl)-6,7-dihydropyrazolo[1,5-α]pyrimidin-5(4H)-one;

2-(4-chloro-1-cyclobutyl-1H-pyrazol-5-yl)-4-(3-fluoro-4-(1-methyl-4-(trifluoromethyl)-1H-imidazol-2-yl)benzyl)-6,7-dihydropyrazolo[1,5-α]pyrimidin-5(4H)-one;

2-(4-chloro-1-isopropyl-1H-pyrazol-5-yl)-4-(3-fluoro-4-(1-isopropyl-4-(trifluoromethyl)-1H-imidazol-2-yl)benzyl)-6,7-dihydropyrazolo[1,5-α]pyrimidin-5(4H)-one;

2-(4-fluoro-1-isopropyl-1H-pyrazol-5-yl)-4-(3-fluoro-4-(1-methyl-4-(trifluoromethyl)-1H-imidazol-2-yl)benzyl)-6,7-dihydropyrazolo[1,5-α]pyrimidin-5(4H)-one;

2-(4-chloro-1-isopropyl-1H-pyrazol-5-yl)-4-(4-(1-ethyl-3-(trifluoromethyl)-1H-1,2,4-triazol-5-yl)benzyl)-6,7-dihydropyrazolo[1,5-α]pyrimidin-5(4H)-one;

4-(4-(1-ethyl-4-(trifluoromethyl)-1H-imidazol-2-yl)benzyl)-2-(1-methylindolin-7-yl)-6,7-dihydropyrazolo[1,5-α]pyrimidin-5(4H)-one;

2-(4-cyclopropyl-6-methoxypyrimidin-5-yl)-4-(4-(1-methyl-4-(trifluoromethyl)-1H-imidazol-2-yl)benzyl)-4,5,6,7-tetrahydropyrazolo[1,5-α]pyrimidine;

2-(4-methoxy-6-methylpyrimidin-5-yl)-4-(4-(1-methyl-4-(trifluoromethyl)-1H-imidazol-2-yl)benzyl)-4,5,6,7-tetrahydropyrazolo[1,5-α]pyrimidine;

2-(4-methoxy-6-methylpyrimidin-5-yl)-4-(4-(5-methyl-3-(trifluoromethyl)-1H-pyrazol-1-yl)benzyl)-4,5,6,7-tetrahydropyrazolo[1,5-α]pyrimidine;

2-(4-cyclopropyl-6-methoxypyrimidin-5-yl)-4-(4-(5-methyl-3-(trifluoromethyl)-1H-pyrazol-1-yl)benzyl)-4,5,6,7-tetrahydropyrazolo[1,5-α]pyrimidine;

2-(4-cyclopropyl-6-methoxypyrimidin-5-yl)-4-(4-(1-ethyl-4-(trifluoromethyl)-1H-imidazol-2-yl)benzyl)-4,5,6,7-tetrahydropyrazolo[1,5-α]pyrimidine;

2-(4-chloro-1-isopropyl-1H-pyrazol-5-yl)-4-(4-(1-ethyl-4-(trifluoromethyl)-1H-imidazol-2-yl)-3-fluorobenzyl)-6-methyl-4,5,6,7-tetrahydropyrazolo[1,5-α]pyrimidine;

2′-(4-cyclopropyl-6-methoxypyrimidin-5-yl)-4′-(4-(1-ethyl-4-(trifluoromethyl)-1H-imidazol-2-yl)-3-fluorobenzyl)-4′,5′-dihydro-7′H-spiro[cyclopropane-1,6′-pyrazolo[1,5-α]pyrimidine];

2-(1-isopropyl-4-methyl-1H-pyrazol-5-yl)-6-methyl-4-(4-(1-methyl-4-(trifluoromethyl)-1H-imidazol-2-yl)benzyl)-6,7-dihydropyrazolo[1,5-α]pyrimidin-5(4H)-one;

2-(4-cyclopropyl-6-methoxypyrimidin-5-yl)-6-methyl-4-(4-(1-methyl-4-(trifluoromethyl)-1H-imidazol-2-yl)benzyl)-6,7-dihydropyrazolo[1,5-α]pyrimidin-5(4H)-one;

3-chloro-2-(4-cyclopropyl-6-methoxypyrimidin-5-yl)-4-(4-(1-methyl-4-(trifluoromethyl)-1H-imidazol-2-yl)benzyl)-6,7-dihydropyrazolo[1,5-α]pyrimidin-5(4H)-one;

3-chloro-2-(1-isopropyl-4-methyl-1H-pyrazol-5-yl)-4-(4-(1-methyl-4-(trifluoromethyl)-1H-imidazol-2-yl)benzyl)-6,7-dihydropyrazolo[1,5-α]pyrimidin-5(4H)-one;

2-(4-cyclopropyl-6-methoxypyrimidin-5-yl)-3-methyl-4-(4-(1-methyl-4-(trifluoromethyl)-1H-imidazol-2-yl)benzyl)-6,7-dihydropyrazolo[1,5-α]pyrimidin-5(4H)-one;

2-(1-isopropyl-4-methyl-1H-pyrazol-5-yl)-7-methyl-4-(4-(1-methyl-4-(trifluoromethyl)-1H-imidazol-2-yl)benzyl)-6,7-dihydropyrazolo[1,5-α]pyrimidin-5(4H)-one;

2-(1-isopropyl-4-methyl-1H-pyrazol-5-yl)-4-(4-(1-methyl-4-(trifluoromethyl)-1H-imidazol-2-yl)benzyl)-6,7-dihydro-[1,2,4]triazolo[1,5-α]pyrimidin-5(4H)-one;

4-(4-(1-isopropyl-4-(trifluoromethyl)-1H-imidazol-2-yl)benzyl)-2-(1-isopropyl-4-methyl-1H-pyrazol-5-yl)-6,7-dihydro-[1,2,4]triazolo[1,5-α]pyrimidin-5(4H)-one;

2-(4-chloro-1-isopropyl-1H-pyrazol-5-yl)-4-(4-(1-methyl-4-(trifluoromethyl)-1H-imidazol-2-yl)benzyl)-6,7-dihydro-[1,2,4]triazolo[1,5-α]pyrimidin-5(4H)-one;

2-(4-chloro-1-isopropyl-1H-pyrazol-5-yl)-4-(3-fluoro-4-(1-isopropyl-4-(trifluoromethyl)-1H-imidazol-2-yl)benzyl)-6,7-dihydro-[1,2,4]triazolo[1,5-α]pyrimidin-5(4H)-one;

2-(4-chloro-1-isopropyl-1H-pyrazol-5-yl)-4-(4-(1-isopropyl-4-(trifluoromethyl)-1H-imidazol-2-yl)benzyl)-6,7-dihydro-[1,2,4]triazolo[1,5-α]pyrimidin-5(4H)-one;

2-(4-chloro-1-isopropyl-1H-pyrazol-5-yl)-4-(4-(1-ethyl-4-(trifluoromethyl)-1H-imidazol-2-yl)-3-fluorobenzyl)-6,7-dihydro-[1,2,4]triazolo[1,5-α]pyrimidin-5(4H)-one;

2-(4-cyclopropyl-6-methoxypyrimidin-5-yl)-4-(4-(1-methyl-4-(trifluoromethyl)-1H-imidazol-2-yl)benzyl)-6,7-dihydro-[1,2,4]triazolo[1,5-α]pyrimidin-5(4H)-one;

2-(4-cyclopropyl-6-methoxypyrimidin-5-yl)-4-(4-(1-ethyl-4-(trifluoromethyl)-1H-imidazol-2-yl)-3-fluorobenzyl)-6,7-dihydro-[1,2,4]triazolo[1,5-α]pyrimidin-5(4H)-one;

4-(4-(1-ethyl-4-(trifluoromethyl)-1H-imidazol-2-yl)benzyl)-2-(1-isopropyl-4-methoxy-1H-pyrazol-5-yl)-6,7-dihydro-[1,2,4]triazolo[1,5-α]pyrimidin-5(4H)-one;

4-(4-(1-isopropyl-4-(trifluoromethyl)-1H-imidazol-2-yl)-3-methoxybenzyl)-2-(4-isopropyl-6-methoxypyrimidin-5-yl)-6,7-dihydro-[1,2,4]triazolo[1,5-α]pyrimidin-5(4H)-one;

2-(1-isopropyl-4-methyl-1H-pyrazol-5-yl)-4-(4-(1-methyl-4-(trifluoromethyl)-1H-imidazol-2-yl)benzyl)-4,5,6,7-tetrahydropyrazolo[1,5-α]pyrimidine;

2-(4-cyclopropyl-6-methoxypyrimidin-5-yl)-5-methyl-4-(4-(1-methyl-4-(trifluoromethyl)-1H-imidazol-2-yl)benzyl)-4,5,6,7-tetrahydropyrazolo[1,5-α]pyrimidine;

2-(4-cyclopropyl-6-methoxypyrimidin-5-yl)-4-(4-(1-ethyl-4-(trifluoromethyl)-1H-imidazol-2-yl)-3-fluorobenzyl)-5-methyl-4,5,6,7-tetrahydropyrazolo[1,5-α]pyrimidine;

2-(4-chloro-1-isopropyl-1H-pyrazol-5-yl)-4-(3-fluoro-4-(1-isopropyl-4-(trifluoromethyl)-1H-imidazol-2-yl)benzyl)-5-methyl-4,5,6,7-tetrahydropyrazolo[1,5-α]pyrimidine;

2-(4-cyclopropyl-6-methoxypyrimidin-5-yl)-7-methyl-4-(4-(1-methyl-4-(trifluoromethyl)-1H-imidazol-2-yl)benzyl)-4,5,6,7-tetrahydropyrazolo[1,5-α]pyrimidine;

2-(1-isopropyl-4-methyl-1H-pyrazol-5-yl)-4-(4-(5-methyl-3-(trifluoromethyl)-1H-pyrazol-1-yl)benzyl)-4,5,6,7-tetrahydropyrazolo[1,5-α]pyrimidine;

2-(1-isopropyl-4-methyl-1H-pyrazol-5-yl)-4-(4-(1-methyl-3-(trifluoromethyl)-1H-1,2,4-triazol-5-yl)benzyl)-4,5,6,7-tetrahydropyrazolo[1,5-α]pyrimidine;

4-(4-(1-isopropyl-4-(trifluoromethyl)-1H-imidazol-2-yl)benzyl)-2-(1-isopropyl-4-methyl-1H-pyrazol-5-yl)-4,5,6,7-tetrahydropyrazolo[1,5-α]pyrimidine;

4-(3-fluoro-4-(1-methyl-4-(trifluoromethyl)-1H-imidazol-2-yl)benzyl)-2-(1-isopropyl-4-methyl-1H-pyrazol-5-yl)-4,5,6,7-tetrahydropyrazolo[1,5-α]pyrimidine;

4-(3-fluoro-4-(5-methyl-3-(trifluoromethyl)-1H-pyrazol-1-yl)benzyl)-2-(1-isopropyl-4-methyl-1H-pyrazol-5-yl)-4,5,6,7-tetrahydropyrazolo[1,5-α]pyrimidine;

4-(3-fluoro-4-(5-methoxy-3-(trifluoromethyl)-1H-pyrazol-1-yl)benzyl)-2-(1-isopropyl-4-methyl-1H-pyrazol-5-yl)-4,5,6,7-tetrahydropyrazolo[1,5-α]pyrimidine;

2-(4-cyclopropyl-6-methoxypyrimidin-5-yl)-4-(4-(1-isopropyl-4-(trifluoromethyl)-1H-imidazol-2-yl)benzyl)-4,5,6,7-tetrahydropyrazolo[1,5-α]pyrimidine;

2-(4-chloro-1-isopropyl-1H-pyrazol-5-yl)-4-(3-fluoro-4-(1-isopropyl-4-(trifluoromethyl)-1H-imidazol-2-yl)benzyl)-4,5,6,7-tetrahydropyrazolo[1,5-α]pyrimidine;

2-(4-chloro-1-isopropyl-1H-pyrazol-5-yl)-4-(4-(1-ethyl-4-(trifluoromethyl)-1H-imidazol-2-yl)-3-fluorobenzyl)-4,5,6,7-tetrahydropyrazolo[1,5-α]pyrimidine;

2-(4-chloro-1-isopropyl-1H-pyrazol-5-yl)-4-(3-fluoro-4-(1-methyl-4-(trifluoromethyl)-1H-imidazol-2-yl)benzyl)-4,5,6,7-tetrahydropyrazolo[1,5-α]pyrimidine;

2-(4-chloro-1-isopropyl-1H-pyrazol-5-yl)-4-(4-(1-(2-fluoroethyl)-4-(trifluoromethyl)-1H-imidazol-2-yl)benzyl)-4,5,6,7-tetrahydropyrazolo[1,5-α]pyrimidine;

2-(4-cyclopropyl-6-methoxypyrimidin-5-yl)-4-(3-fluoro-4-(1-isopropyl-4-(trifluoromethyl)-1H-imidazol-2-yl)benzyl)-4,5,6,7-tetrahydropyrazolo[1,5-α]pyrimidine;

2-(4-cyclopropyl-6-methoxypyrimidin-5-yl)-4-(4-(1-ethyl-4-(trifluoromethyl)-1H-imidazol-2-yl)-3-fluorobenzyl)-4,5,6,7-tetrahydropyrazolo[1,5-α]pyrimidine;

2-(4-chloro-1-isopropyl-1H-pyrazol-5-yl)-4-(1-(4-(1-ethyl-4-(trifluoromethyl)-1H-imidazol-2-yl)phenyl)ethyl)-4,5,6,7-tetrahydropyrazolo[1,5-α]pyrimidine;

2-(4-cyclopropyl-6-methoxypyrimidin-5-yl)-4-(1-(4-(1-ethyl-4-(trifluoromethyl)-1H-imidazol-2-yl)phenyl)ethyl)-4,5,6,7-tetrahydropyrazolo[1,5-α]pyrimidine;

(S)-2-(4-chloro-1-isopropyl-1H-pyrazol-5-yl)-4-(1-(4-(1-ethyl-4-(trifluoromethyl)-1H-imidazol-2-yl)phenyl)ethyl)-4,5,6,7-tetrahydropyrazolo[1,5-α]pyrimidine;

(R)-2-(4-chloro-1-isopropyl-1H-pyrazol-5-yl)-4-(1-(4-(1-ethyl-4-(trifluoromethyl)-1H-imidazol-2-yl)phenyl)ethyl)-4,5,6,7-tetrahydropyrazolo[1,5-α]pyrimidine;

2-(4-cyclopropyl-6-methoxypyrimidin-5-yl)-4-(4-(4-(trifluoromethyl)-1H-imidazol-2-yl)benzyl)-6,7-dihydropyrazolo[1,5-α]pyrimidin-5(4H)-one;

2-(1-isopropyl-4-methyl-1H-pyrazol-5-yl)-4-(4-(4-(trifluoromethyl)-1H-imidazol-2-yl)benzyl)-6,7-dihydropyrazolo[1,5-α]pyrimidin-5(4H)-one;

2-(4-cyclopropyl-6-methoxypyrimidin-5-yl)-4-(4-(1-ethyl-4-(trifluoromethyl)-1H-imidazol-2-yl)benzyl)-6,7-dihydropyrazolo[1,5-α]pyrimidin-5(4H)-one;

2-(4-cyclopropyl-6-methoxypyrimidin-5-yl)-4-(4-(1-isopropyl-4-(trifluoromethyl)-1H-imidazol-2-yl)benzyl)-6,7-dihydropyrazolo[1,5-α]pyrimidin-5(4H)-one;

4-(4-(1-isopropyl-4-(trifluoromethyl)-1H-imidazol-2-yl)benzyl)-2-(1-isopropyl-4-methyl-1H-pyrazol-5-yl)-6,7-dihydropyrazolo[1,5-α]pyrimidin-5(4H)-one;

2-(2-methoxy-6-methylphenyl)-4-(4-(1-methyl-4-(trifluoromethyl)-1H-imidazol-2-yl)benzyl)-4,5,6,7-tetrahydropyrazolo[1,5-α]pyrimidine;

2-(2-methoxy-6-methylphenyl)-4-(4-(1-methyl-4-(trifluoromethyl)-1H-imidazol-2-yl)benzyl)-6,7-dihydropyrazolo[1,5-α]pyrimidin-5(4H)-one;

2-(4-cyclopropyl-6-methoxypyrimidin-5-yl)-6,6-dimethyl-4-(4-(1-methyl-4-(trifluoromethyl)-1H-imidazol-2-yl)benzyl)-6,7-dihydropyrazolo[1,5-α]pyrimidin-5(4H)-one;

2-(4-chloro-1-isopropyl-1H-pyrazol-5-yl)-4-(4-(1-ethyl-4-(trifluoromethyl)-1H-imidazol-2-yl)-3-fluorobenzyl)-6,7-dihydropyrazolo[1,5-α]pyrimidin-5(4H)-one;

2-(4-cyclopropyl-6-methoxypyrimidin-5-yl)-4-(4-(1-ethyl-4-(trifluoromethyl)-1H-imidazol-2-yl)-3-fluorobenzyl)-6,7-dihydropyrazolo[1,5-α]pyrimidin-5(4H)-one;

2-(4-chloro-1-isopropyl-1H-pyrazol-5-yl)-4-(4-(1-(2-fluoroethyl)-4-(trifluoromethyl)-1H-imidazol-2-yl)benzyl)-6,7-dihydropyrazolo[1,5-α]pyrimidin-5(4H)-one;

2-(4-cyclopropyl-6-methoxypyrimidin-5-yl)-4-(4-(5-methoxy-3-(trifluoromethyl)-1H-pyrazol-1-yl)benzyl)-6,7-dihydropyrazolo[1,5-α]pyrimidin-5(4H)-one;

2-(4-cyclopropyl-6-methoxypyrimidin-5-yl)-4-(4-(1-(2-fluoroethyl)-4-(trifluoromethyl)-1H-imidazol-2-yl)benzyl)-6,7-dihydropyrazolo[1,5-α]pyrimidin-5(4H)-one;

2-(4-chloro-1-isopropyl-1H-pyrazol-5-yl)-4-((6-(1-methyl-4-(trifluoromethyl)-1H-imidazol-2-yl)pyridin-3-yl)methyl)-6,7-dihydropyrazolo[1,5-α]pyrimidin-5(4H)-one;

2-(4-cyclopropyl-6-methoxypyrimidin-5-yl)-4-((6-(1-methyl-4-(trifluoromethyl)-1H-imidazol-2-yl)pyridin-3-yl)methyl)-6,7-dihydropyrazolo[1,5-α]pyrimidin-5(4H)-one;

2-(4-chloro-1-isopropyl-1H-pyrazol-5-yl)-4-(4-(1-ethyl-3-(trifluoromethyl)-1H-1,2,4-triazol-5-yl)benzyl)-6,7-dihydropyrazolo[1,5-α]pyrimidin-5(4H)-one;

4-(4-(1-ethyl-4-(trifluoromethyl)-1H-imidazol-2-yl)benzyl)-2-(4-fluoro-1-isopropyl-1H-pyrazol-5-yl)-6,7-dihydropyrazolo[1,5-α]pyrimidin-5(4H)-one;

2-(4-chloro-1-isopropyl-1H-pyrazol-5-yl)-4-(4-(5-methyl-3-(trifluoromethyl)-1H-pyrazol-1-yl)benzyl)-6,7-dihydropyrazolo[1,5-α]pyrimidin-5(4H)-one;

2-(4-cyclopropyl-6-methoxypyrimidin-5-yl)-4-(4-(1-ethyl-3-(trifluoromethyl)-1H-1,2,4-triazol-5-yl)benzyl)-6,7-dihydropyrazolo[1,5-α]pyrimidin-5(4H)-one;

2-(4-cyclopropyl-6-methoxypyrimidin-5-yl)-4-(3-fluoro-4-(3-methyl-5-(trifluoromethyl)-1H-pyrazol-1-yl)benzyl)-6,7-dihydropyrazolo[1,5-α]pyrimidin-5(4H)-one;

2-(4-chloro-1-isopropyl-1H-pyrazol-5-yl)-4-((6-(5-methoxy-3-(trifluoromethyl)-1H-pyrazol-1-yl)pyridin-3-yl)methyl)-6,7-dihydropyrazolo[1,5-α]pyrimidin-5(4H)-one;

2-(4-cyclopropyl-6-methoxypyrimidin-5-yl)-4-(4-(1,4-dimethyl-1H-pyrazol-3-yl)benzyl)-6,7-dihydropyrazolo[1,5-α]pyrimidin-5(4H)-one;

2-(4-chloro-1-isopropyl-1H-pyrazol-5-yl)-4-(4-(1-isopropyl-4-(trifluoromethyl)-1H-imidazol-2-yl)benzyl)-6,7-dihydropyrazolo[1,5-α]pyrimidin-5(4H)-one;

2-(4-chloro-1-isopropyl-1H-pyrazol-5-yl)-4-(3-fluoro-4-(3-methyl-5-(trifluoromethyl)-1H-pyrazol-1-yl)benzyl)-6,7-dihydropyrazolo[1,5-α]pyrimidin-5(4H)-one;

2-(4-chloro-1-isopropyl-1H-pyrazol-5-yl)-4-(4-(1,4-dimethyl-1H-pyrazol-3-yl)benzyl)-6,7-dihydropyrazolo[1,5-α]pyrimidin-5(4H)-one;

2-(4-cyclopropyl-6-methoxypyrimidin-5-yl)-4-((6-(5-methoxy-3-(trifluoromethyl)-1H-pyrazol-1-yl)pyridin-3-yl)methyl)-6,7-dihydropyrazolo[1,5-α]pyrimidin-5(4H)-one;

2-(4-chloro-1-isopropyl-1H-pyrazol-5-yl)-4-(4-(1-methyl-1H-pyrazol-3-yl)benzyl)-6,7-dihydropyrazolo[1,5-α]pyrimidin-5(4H)-one;

2-(4-chloro-1-isopropyl-1H-pyrazol-5-yl)-4-(4-(5-methoxy-3-(trifluoromethyl)-1H-pyrazol-1-yl)benzyl)-6,7-dihydropyrazolo[1,5-α]pyrimidin-5(4H)-one;

2-(4-chloro-1-isopropyl-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one;

2-(4-chloro-1-isopropyl-1H-pyrazol-5-yl)-4-(4-(1-methyl-4-(trifluoromethyl)-1H-imidazol-2-yl)benzyl)-6,7-dihydropyrazolo[1,5-α]pyrimidin-5(4H)-one;

2-(4-cyclopropyl-6-methoxypyrimidin-5-yl)-4-(4-(5-(methoxymethyl)-3-(trifluoromethyl)-1H-pyrazol-1-yl)benzyl)-6,7-dihydropyrazolo[1,5-α]pyrimidin-5(4H)-one;

2-(4-chloro-1-isopropyl-1H-pyrazol-5-yl)-4-(4-(5-(methoxymethyl)-3-(trifluoromethyl)-1H-pyrazol-1-yl)benzyl)-6,7-dihydropyrazolo[1,5-α]pyrimidin-5(4H)-one;

2-(4-chloro-1-isopropyl-1H-pyrazol-5-yl)-4-(4-(1-methyl-5-(trifluoromethyl)-1H-1,2,4-triazol-3-yl)benzyl)-6,7-dihydropyrazolo[1,5-α]pyrimidin-5(4H)-one;

2-(4-chloro-1-isopropyl-1H-pyrazol-5-yl)-4-(4-(1-methyl-3-(trifluoromethyl)-1H-1,2,4-triazol-5-yl)benzyl)-6,7-dihydropyrazolo[1,5-α]pyrimidin-5(4H)-one;

2-(4-cyclopropyl-6-methoxypyrimidin-5-yl)-4-(3-fluoro-4-(5-methyl-3-(trifluoromethyl)-1H-pyrazol-1-yl)benzyl)-6,7-dihydropyrazolo[1,5-α]pyrimidin-5(4H)-one;

2-(4-chloro-1-isopropyl-1H-pyrazol-5-yl)-4-(3-fluoro-4-(5-methyl-3-(trifluoromethyl)-1H-pyrazol-1-yl)benzyl)-6,7-dihydropyrazolo[1,5-α]pyrimidin-5(4H)-one;

2-(4-cyclopropyl-6-methoxypyrimidin-5-yl)-4-((6-(1-ethyl-4-(trifluoromethyl)-1H-imidazol-2-yl)pyridin-3-yl)methyl)-6,7-dihydropyrazolo[1,5-α]pyrimidin-5(4H)-one;

1-(4-((2-(4-chloro-1-isopropyl-1H-pyrazol-5-yl)-5-oxo-6,7-dihydropyrazolo[1,5-α]pyrimidin-4(5H)-yl)methyl)phenyl)-5-methoxy-1H-pyrazole-3-carbonitrile;

1-(4-((2-(4-cyclopropyl-6-methoxypyrimidin-5-yl)-5-oxo-6,7-dihydropyrazolo[1,5-α]pyrimidin-4(5H)-yl)methyl)phenyl)-5-methoxy-1H-pyrazole-3-carbonitrile;

2-(4-cyclopropyl-6-methoxypyrimidin-5-yl)-4-(4-(1-isopropyl-4-(trifluoromethyl)-1H-imidazol-2-yl)-3-methoxybenzyl)-6,7-dihydropyrazolo[1,5-α]pyrimidin-5(4H)-one;

2-(4-chloro-1-isopropyl-1H-pyrazol-5-yl)-4-(4-(1-isopropyl-4-(trifluoromethyl)-1H-imidazol-2-yl)-3-methoxybenzyl)-6,7-dihydropyrazolo[1,5-α]pyrimidin-5(4H)-one;

2-(4-chloro-1-isopropyl-1H-pyrazol-5-yl)-4-(1-(4-fluorophenyl)ethyl)-6,7-dihydropyrazolo[1,5-α]pyrimidin-5(4H)-one;

2-(4-chloro-1-isopropyl-1H-pyrazol-5-yl)-4-((6-(1-ethyl-4-(trifluoromethyl)-1H-imidazol-2-yl)pyridin-3-yl)methyl)-6,7-dihydropyrazolo[1,5-α]pyrimidin-5(4H)-one;

2-(4-chloro-1-isopropyl-1H-pyrazol-5-yl)-4-(4-(1-isopropyl-4-nitro-1H-imidazol-2-yl)benzyl)-6,7-dihydropyrazolo[1,5-α]pyrimidin-5(4H)-one;

2-(4-chloro-1-isopropyl-1H-pyrazol-5-yl)-4-(1-(4-(1-ethyl-4-(trifluoromethyl)-1H-imidazol-2-yl)phenyl)ethyl)-6,7-dihydropyrazolo[1,5-α]pyrimidin-5(4H)-one;

2-(4-cyclopropyl-6-methoxypyrimidin-5-yl)-4-(1-(4-(1-ethyl-4-(trifluoromethyl)-1H-imidazol-2-yl)phenyl)ethyl)-6,7-dihydropyrazolo[1,5-α]pyrimidin-5(4H)-one;

2-(4-chloro-1-isopropyl-1H-pyrazol-5-yl)-4-(4-(5-ethoxy-3-(trifluoromethyl)-1H-pyrazol-1-yl)benzyl)-6,7-dihydropyrazolo[1,5-α]pyrimidin-5(4H)-one;

2-(4-chloro-1-isopropyl-1H-pyrazol-5-yl)-4-(4-(1-methyl-2-(trifluoromethyl)-1H-imidazol-4-yl)benzyl)-6,7-dihydropyrazolo[1,5-α]pyrimidin-5(4H)-one;

2-(4-chloro-1-isopropyl-1H-pyrazol-5-yl)-4-(4-(5-methoxy-1H-pyrazol-1-yl)benzyl)-6,7-dihydropyrazolo[1,5-α]pyrimidin-5(4H)-one;

4-(4-(4-chloro-1-ethyl-1H-imidazol-2-yl)benzyl)-2-(4-chloro-1-isopropyl-1H-pyrazol-5-yl)-6,7-dihydropyrazolo[1,5-α]pyrimidin-5(4H)-one;

4-(4-(4-chloro-1-ethyl-1H-imidazol-5-yl)benzyl)-2-(4-chloro-1-isopropyl-1H-pyrazol-5-yl)-6,7-dihydropyrazolo[1,5-α]pyrimidin-5(4H)-one;

2-(4-chloro-1-isopropyl-1H-pyrazol-5-yl)-4-(1-(4-(1-ethyl-4-(trifluoromethyl)-1H-imidazol-2-yl)-3-fluorophenyl)ethyl)-6,7-dihydropyrazolo[1,5-α]pyrimidin-5(4H)-one;

2-(4-cyclopropyl-6-methoxypyrimidin-5-yl)-7-methyl-4-(4-(1-methyl-4-(trifluoromethyl)-1H-imidazol-2-yl)benzyl)-6,7-dihydropyrazolo[1,5-α]pyrimidin-5(4H)-one;

2-(4-cyclopropyl-6-methoxypyrimidin-5-yl)-4-(1-(4-(1-ethyl-4-(trifluoromethyl)-1H-imidazol-2-yl)-3-fluorophenyl)ethyl)-6,7-dihydropyrazolo[1,5-α]pyrimidin-5(4H)-one;

2-(4-chloro-1-isopropyl-1H-pyrazol-5-yl)-4-(5-(1-ethyl-4-(trifluoromethyl)-1H-imidazol-2-yl)-6-fluoro-2,3-dihydro-1H-inden-1-yl)-6,7-dihydropyrazolo[1,5-α]pyrimidin-5(4H)-one;

(S)-2-(4-cyclopropyl-6-methoxypyrimidin-5-yl)-4-(1-(4-(1-isopropyl-4-(trifluoromethyl)-1H-imidazol-2-yl)phenyl)ethyl)-6,7-dihydropyrazolo[1,5-α]pyrimidin-5(4H)-one;

(R)-2-(4-cyclopropyl-6-methoxypyrimidin-5-yl)-4-(1-(4-(1-isopropyl-4-(trifluoromethyl)-1H-imidazol-2-yl)phenyl)ethyl)-6,7-dihydropyrazolo[1,5-α]pyrimidin-5(4H)-one;

(S)-2-(4-cyclopropyl-6-methoxypyrimidin-5-yl)-4-(1-(4-(1-ethyl-4-(trifluoromethyl)-1H-imidazol-2-yl)phenyl)ethyl)-6,7-dihydropyrazolo[1,5-α]pyrimidin-5(4H)-one;

(R)-2-(4-cyclopropyl-6-methoxypyrimidin-5-yl)-4-(1-(4-(1-ethyl-4-(trifluoromethyl)-1H-imidazol-2-yl)phenyl)ethyl)-6,7-dihydropyrazolo[1,5-α]pyrimidin-5(4H)-one;

(S)-2-(4-chloro-1-isopropyl-1H-pyrazol-5-yl)-4-(1-(4-(1-ethyl-4-(trifluoromethyl)-1H-imidazol-2-yl)phenyl)ethyl)-6,7-dihydropyrazolo[1,5-α]pyrimidin-5(4H)-one;

(R)-2-(4-chloro-1-isopropyl-1H-pyrazol-5-yl)-4-(1-(4-(1-ethyl-4-(trifluoromethyl)-1H-imidazol-2-yl)phenyl)ethyl)-6,7-dihydropyrazolo[1,5-α]pyrimidin-5(4H)-one;

(S)-2-(4-cyclopropyl-6-methoxypyrimidin-5-yl)-4-(1-(4-(1-isopropyl-4-(trifluoromethyl)-1H-imidazol-2-yl)-3-methoxyphenyl)ethyl)-6,7-dihydropyrazolo[1,5-α]pyrimidin-5(4H)-one;

(R)-2-(4-cyclopropyl-6-methoxypyrimidin-5-yl)-4-(1-(4-(1-isopropyl-4-(trifluoromethyl)-1H-imidazol-2-yl)-3-methoxyphenyl)ethyl)-6,7-dihydropyrazolo[1,5-α]pyrimidin-5(4H)-one;

2-(4-chloro-1-isopropyl-1H-pyrazol-5-yl)-4-(3-fluoro-4-(1-methyl-2-(trifluoromethyl)-1H-imidazol-4-yl)benzyl)-6,7-dihydropyrazolo[1,5-α]pyrimidin-5(4H)-one;

(R)-2-(4-chloro-1-(1-methoxypropan-2-yl)-1H-pyrazol-5-yl)-4-(4-(1-ethyl-4-(trifluoromethyl)-1H-imidazol-2-yl)-3-fluorobenzyl)-6,7-dihydropyrazolo[1,5-α]pyrimidin-5(4H)-one;

(S)-2-(4-chloro-1-(1-methoxypropan-2-yl)-1H-pyrazol-5-yl)-4-(4-(1-ethyl-4-(trifluoromethyl)-1H-imidazol-2-yl)-3-fluorobenzyl)-6,7-dihydropyrazolo[1,5-α]pyrimidin-5(4H)-one;

3-(4-((2-(4-chloro-1-isopropyl-1H-pyrazol-5-yl)-5-oxo-6,7-dihydropyrazolo[1,5-α]pyrimidin-4(5H)-yl)methyl)phenyl)-1-methyl-1H-pyrazole-4-carbonitrile;

2-(4-chloro-1-isopropyl-1H-pyrazol-5-yl)-4-(4-(1-ethyl-4-methyl-1H-imidazol-2-yl)benzyl)-6,7-dihydropyrazolo[1,5-α]pyrimidin-5(4H)-one;

2-(4-chloro-1-isopropyl-1H-pyrazol-5-yl)-4-(4-(5-methoxy-3-methyl-1H-pyrazol-1-yl)benzyl)-6,7-dihydropyrazolo[1,5-α]pyrimidin-5(4H)-one;

2-(4-cyclopropyl-6-methoxypyrimidin-5-yl)-4-(4-(1-ethyl-4-(trifluoromethyl)-1H-imidazol-2-yl)-3-methoxybenzyl)-6,7-dihydropyrazolo[1,5-α]pyrimidin-5(4H)-one;

2-(4-cyclopropyl-6-methoxypyrimidin-5-yl)-4-((5-(1-ethyl-4-(trifluoromethyl)-1H-imidazol-2-yl)pyridin-2-yl)methyl)-6,7-dihydropyrazolo[1,5-α]pyrimidin-5(4H)-one;

(S)-2-(4-chloro-1-isopropyl-1H-pyrazol-5-yl)-4-(1-(6-(1-ethyl-4-(trifluoromethyl)-1H-imidazol-2-yl)pyridin-3-yl)ethyl)-6,7-dihydropyrazolo[1,5-α]pyrimidin-5(4H)-one;

(R)-2-(4-chloro-1-isopropyl-1H-pyrazol-5-yl)-4-(1-(6-(1-ethyl-4-(trifluoromethyl)-1H-imidazol-2-yl)pyridin-3-yl)ethyl)-6,7-dihydropyrazolo[1,5-α]pyrimidin-5(4H)-one;

2-(4-cyclopropyl-6-methoxypyrimidin-5-yl)-4-(4-(1-ethyl-4-(trifluoromethyl)-1H-imidazol-2-yl)-3-fluoro-5-methoxybenzyl)-6,7-dihydropyrazolo[1,5-α]pyrimidin-5(4H)-one;

2-(4-chloro-1-isopropyl-1H-pyrazol-5-yl)-4-(4-(1-ethyl-4-(trifluoromethyl)-1H-imidazol-2-yl)-3-fluoro-5-methoxybenzyl)-6,7-dihydropyrazolo[1,5-α]pyrimidin-5(4H)-one;

(S)-2-(4-cyclopropyl-6-methoxypyrimidin-5-yl)-4-(1-(4-(1-ethyl-4-(trifluoromethyl)-1H-imidazol-2-yl)-3-fluorophenyl)ethyl)-6,7-dihydropyrazolo[1,5-α]pyrimidin-5(4H)-one;

(R)-2-(4-cyclopropyl-6-methoxypyrimidin-5-yl)-4-(1-(4-(1-ethyl-4-(trifluoromethyl)-1H-imidazol-2-yl)-3-fluorophenyl)ethyl)-6,7-dihydropyrazolo[1,5-α]pyrimidin-5(4H)-one;

2-(4-cyclopropyl-6-methoxypyrimidin-5-yl)-4-(3-fluoro-4-(1-isopropyl-4-(trifluoromethyl)-1H-imidazol-2-yl)-5-methoxybenzyl)-6,7-dihydropyrazolo[1,5-α]pyrimidin-5(4H)-one;

4-(3-chloro-4-(1-ethyl-4-(trifluoromethyl)-1H-imidazol-2-yl)-5-methoxybenzyl)-2-(4-cyclopropyl-6-methoxypyrimidin-5-yl)-6,7-dihydropyrazolo[1,5-α]pyrimidin-5(4H)-one;

2-(4-chloro-1-isopropyl-1H-pyrazol-5-yl)-4-(4-(3-methoxy-5-methyl-1H-pyrazol-1-yl)benzyl)-6,7-dihydropyrazolo[1,5-α]pyrimidin-5(4H)-one;

3-chloro-2-(1-isopropyl-4-methyl-1H-pyrazol-5-yl)-4-(4-(1-methyl-4-(trifluoromethyl)-1H-imidazol-2-yl)benzyl)-4,5,6,7-tetrahydropyrazolo[1,5-α]pyrimidine;

3-chloro-4-(3-fluoro-4-(1-isopropyl-4-(trifluoromethyl)-1H-imidazol-2-yl)benzyl)-2-(1-isopropyl-4-methyl-1H-pyrazol-5-yl)-4,5,6,7-tetrahydropyrazolo[1,5-α]pyrimidine;

4-(3-fluoro-4-(1-methyl-4-(trifluoromethyl)-1H-imidazol-2-yl)benzyl)-2-(4-methoxy-6-(1-methylcyclopropyl)pyrimidin-5-yl)-6,7-dihydropyrazolo[1,5-α]pyrimidin-5(4H)-one;

2-(4-chloro-1-(tetrahydrofuran-3-yl)-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one;

2-(4-chloro-1-(1-methoxypropan-2-yl)-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one;

4-(4-(1-isopropyl-4-(trifluoromethyl)-1H-imidazol-2-yl)benzyl)-2-(1-isopropyl-4-methoxy-1H-pyrazol-5-yl)-6,7-dihydropyrazolo[1,5-α]pyrimidin-5(4H)-one;

2-(4-chloro-1-(1-(methoxymethyl)cyclopropyl)-1H-pyrazol-5-yl)-4-(4-(1-ethyl-4-(trifluoromethyl)-1H-imidazol-2-yl)-3-fluorobenzyl)-6,7-dihydropyrazolo[1,5-α]pyrimidin-5(4H)-one;

2-(4-chloro-1-(oxetan-3-yl)-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one;

4-(4-(1-ethyl-4-(trifluoromethyl)-1H-imidazol-2-yl)benzyl)-2-(1-isopropyl-4-methoxy-1H-pyrazol-5-yl)-6,7-dihydropyrazolo[1,5-α]pyrimidin-5(4H)-one;

5-(4-(4-(1-ethyl-4-(trifluoromethyl)-1H-imidazol-2-yl)benzyl)-5-oxo-4,5,6,7-tetrahydropyrazolo[1,5-α]pyrimidin-2-yl)-1-isopropyl-1H-pyrazole-4-carbonitrile;

2-(4-(tert-butyl)-1-methyl-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one;

4-(4-(1-ethyl-4-(trifluoromethyl)-1H-imidazol-2-yl)benzyl)-2-(1-isopropyl-1-methyl-1H-pyrazol-5-yl)-6,7-dihydropyrazolo[1,5-α]pyrimidin-5(4H)-one;

2-(4-chloro-1-(1-(methoxymethyl)cyclopropyl)-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one;

2-(4-chloro-1-(1-methoxypropan-2-yl)-1H-pyrazol-5-yl)-4-(4-(1-ethyl-4-(trifluoromethyl)-1H-imidazol-2-yl)-3-fluorobenzyl)-6,7-dihydropyrazolo[1,5-α]pyrimidin-5(4H)-one;

2-(4-cyclopropyl-1-isopropyl-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one;

2-(4-chloro-1-(1-methoxy-2-methylpropan-2-yl)-1H-pyrazol-5-yl)-4-(4-(1-ethyl-4-(trifluoromethyl)-1H-imidazol-2-yl)-3-fluorobenzyl)-6,7-dihydropyrazolo[1,5-α]pyrimidin-5(4H)-one;

2-(4-chloro-1-(2-hydroxyethyl)-1H-pyrazol-5-yl)-4-(4-(1-ethyl-4-(trifluoromethyl)-1H-imidazol-2-yl)-3-fluorobenzyl)-6,7-dihydropyrazolo[1,5-α]pyrimidin-5(4H)-one;

2-(4-chloro-1-isopropyl-1H-pyrazol-5-yl)-4-(4-(1-ethyl-4-(trifluoromethyl)-1H-imidazol-2-yl)phenoxy)-4,5,6,7-tetrahydropyrazolo[1,5-α]pyridine;

2-(4-chloro-1-isopropyl-1H-pyrazol-5-yl)-4-(4-(1-isopropyl-4-(trifluoromethyl)-1H-imidazol-2-yl)-3-methoxybenzyl)-6,7-dihydro-[1,2,4]triazolo[1,5-α]pyrimidin-5(4H)-one;

2-(4-cyclopropyl-6-methoxypyrimidin-5-yl)-4-(4-(1-isopropyl-4-(trifluoromethyl)-1H-imidazol-2-yl)-3-methoxybenzyl)-6,7-dihydro-[1,2,4]triazolo[1,5-α]pyrimidin-5(4H)-one;

2-(4-chloro-1-isopropyl-1H-pyrazol-5-yl)-4-(4-(1-isopropyl-4-(trifluoromethyl)-1H-imidazol-2-yl)-3-(2-methoxyethoxy)benzyl)-6,7-dihydro-[1,2,4]triazolo[1,5-α]pyrimidin-5(4H)-one;

2-(4-cyclopropyl-6-methoxypyrimidin-5-yl)-4-(4-(1-isopropyl-4-(trifluoromethyl)-1H-imidazol-2-yl)benzyl)-6,7-dihydro-[1,2,4]triazolo[1,5-α]pyrimidin-5(4H)-one;

2-(4-cyclopropyl-6-methoxypyrimidin-5-yl)-4-(1-(4-(1-isopropyl-4-(trifluoromethyl)-1H-imidazol-2-yl)phenyl)ethyl)-6,7-dihydro-[1,2,4]triazolo[1,5-α]pyrimidin-5(4H)-one;

2-(4-cyclopropyl-6-methoxypyrimidin-5-yl)-4-(3-fluoro-4-(1-methyl-4-(trifluoromethyl)-1H-imidazol-2-yl)benzyl)-6,7-dihydro-[1,2,4]triazolo[1,5-α]pyrimidin-5(4H)-one;

2-(4-cyclopropyl-6-methoxypyrimidin-5-yl)-4-(4-(1-ethyl-4-(trifluoromethyl)-1H-imidazol-2-yl)benzyl)-6,7-dihydro-[1,2,4]triazolo[1,5-α]pyrimidin-5(4H)-one;

4-(3-chloro-4-(1-isopropyl-4-(trifluoromethyl)-1H-imidazol-2-yl)benzyl)-2-(4-cyclopropyl-6-methoxypyrimidin-5-yl)-6,7-dihydro-[1,2,4]triazolo[1,5-α]pyrimidin-5(4H)-one;

(S)-2-(4-cyclopropyl-6-methoxypyrimidin-5-yl)-4-(1-(3-fluoro-4-(1-isopropyl-4-(trifluoromethyl)-1H-imidazol-2-yl)phenyl)ethyl)-6,7-dihydro-[1,2,4]triazolo[1,5-α]pyrimidin-5(4H)-one;

(R)-2-(4-cyclopropyl-6-methoxypyrimidin-5-yl)-4-(1-(3-fluoro-4-(1-isopropyl-4-(trifluoromethyl)-1H-imidazol-2-yl)phenyl)ethyl)-6,7-dihydro-[1,2,4]triazolo[1,5-α]pyrimidin-5(4H)-one;

4-(3-chloro-4-(1-ethyl-4-(trifluoromethyl)-1H-imidazol-2-yl)benzyl)-2-(4-cyclopropyl-6-methoxypyrimidin-5-yl)-6,7-dihydro-[1,2,4]triazolo[1,5-α]pyrimidin-5(4H)-one;

2-(4-cyclopropyl-6-methoxypyrimidin-5-yl)-4-(4-(1-ethyl-4-(trifluoromethyl)-1H-imidazol-2-yl)-3-fluoro-5-methoxybenzyl)-6,7-dihydro-[1,2,4]triazolo[1,5-α]pyrimidin-5(4H)-one;

(S)-2-(4-chloro-1-isopropyl-1H-pyrazol-5-yl)-4-(1-(4-(1-isopropyl-4-(trifluoromethyl)-1H-imidazol-2-yl)phenyl)ethyl)-6,7-dihydro-[1,2,4]triazolo[1,5-α]pyrimidin-5(4H)-one;

(R)-2-(4-chloro-1-isopropyl-1H-pyrazol-5-yl)-4-(1-(4-(1-isopropyl-4-(trifluoromethyl)-1H-imidazol-2-yl)phenyl)ethyl)-6,7-dihydro-[1,2,4]triazolo[1,5-α]pyrimidin-5(4H)-one;

(S)-2-(4-chloro-1-isopropyl-1H-pyrazol-5-yl)-4-(1-(4-(1-ethyl-4-(trifluoromethyl)-1H-imidazol-2-yl)-3-methoxyphenyl)ethyl)-6,7-dihydro-[1,2,4]triazolo[1,5-α]pyrimidin-5(4H)-one;

(R)-2-(4-chloro-1-isopropyl-1H-pyrazol-5-yl)-4-(1-(4-(1-ethyl-4-(trifluoromethyl)-1H-imidazol-2-yl)-3-methoxyphenyl)ethyl)-6,7-dihydro-[1,2,4]triazolo[1,5-α]pyrimidin-5(4H)-one;

(S)-2-(4-chloro-1-isopropyl-1H-pyrazol-5-yl)-4-(1-(4-(1-ethyl-4-(trifluoromethyl)-1H-imidazol-2-yl)-3-fluorophenyl)ethyl)-6,7-dihydro-[1,2,4]triazolo[1,5-α]pyrimidin-5(4H)-one;

(R)-2-(4-chloro-1-isopropyl-1H-pyrazol-5-yl)-4-(1-(4-(1-ethyl-4-(trifluoromethyl)-1H-imidazol-2-yl)-3-fluorophenyl)ethyl)-6,7-dihydro-[1,2,4]triazolo[1,5-α]pyrimidin-5(4H)-one;

(S)-2-(4-cyclopropyl-6-methoxypyrimidin-5-yl)-4-(1-(4-(1-ethyl-4-(trifluoromethyl)-1H-imidazol-2-yl)phenyl)ethyl)-6,7-dihydro-[1,2,4]triazolo[1,5-α]pyrimidin-5(4H)-one;

(R)-2-(4-cyclopropyl-6-methoxypyrimidin-5-yl)-4-(1-(4-(1-ethyl-4-(trifluoromethyl)-1H-imidazol-2-yl)phenyl)ethyl)-6,7-dihydro-[1,2,4]triazolo[1,5-α]pyrimidin-5(4H)-one;

2-(4-cyclopropyl-6-methoxypyrimidin-5-yl)-4-(4-(1-ethyl-4-(trifluoromethyl)-1H-imidazol-2-yl)-3-methoxybenzyl)-6,7-dihydro-[1,2,4]triazolo[1,5-α]pyrimidin-5(4H)-one;

2-(4-chloro-1-isopropyl-1H-pyrazol-5-yl)-4-((6-(1-ethyl-4-(trifluoromethyl)-1H-imidazol-2-yl)pyridin-3-yl)methyl)-6,7-dihydro-[1,2,4]triazolo[1,5-α]pyrimidin-5(4H)-one;

2-(4-chloro-1-isopropyl-1H-pyrazol-5-yl)-4-(4-(1-ethyl-4-(trifluoromethyl)-1H-imidazol-2-yl)-3-fluoro-5-methoxybenzyl)-6,7-dihydro-[1,2,4]triazolo[1,5-α]pyrimidin-5(4H)-one;

(S)-2-(4-cyclopropyl-6-methoxypyrimidin-5-yl)-4-(1-(4-(1-isopropyl-4-(trifluoromethyl)-1H-imidazol-2-yl)phenyl)ethyl)-6,7-dihydro-[1,2,4]triazolo[1,5-α]pyrimidin-5(4H)-one;

(R)-2-(4-cyclopropyl-6-methoxypyrimidin-5-yl)-4-(1-(4-(1-isopropyl-4-(trifluoromethyl)-1H-imidazol-2-yl)phenyl)ethyl)-6,7-dihydro-[1,2,4]triazolo[1,5-α]pyrimidin-5(4H)-one;

(S)-2-(4-cyclopropyl-6-methoxypyrimidin-5-yl)-4-(1-(4-(1-ethyl-4-(trifluoromethyl)-1H-imidazol-2-yl)-3-fluorophenyl)ethyl)-6,7-dihydro-[1,2,4]triazolo[1,5-α]pyrimidin-5(4H)-one;

(R)-2-(4-cyclopropyl-6-methoxypyrimidin-5-yl)-4-(1-(4-(1-ethyl-4-(trifluoromethyl)-1H-imidazol-2-yl)-3-fluorophenyl)ethyl)-6,7-dihydro-[1,2,4]triazolo[1,5-α]pyrimidin-5(4H)-one;

2-(4-cyclopropyl-6-methoxypyrimidin-5-yl)-4-(3-(difluoromethoxy)-4-(1-ethyl-4-(trifluoromethyl)-1H-imidazol-2-yl)benzyl)-6,7-dihydro-[1,2,4]triazolo[1,5-α]pyrimidin-5(4H)-one;

(S)-2-(4-chloro-1-isopropyl-1H-pyrazol-5-yl)-4-(1-(4-(1-ethyl-4-(trifluoromethyl)-1H-imidazol-2-yl)phenyl)ethyl)-6,7-dihydro-[1,2,4]triazolo[1,5-α]pyrimidin-5(4H)-one;

(R)-2-(4-chloro-1-isopropyl-1H-pyrazol-5-yl)-4-(1-(4-(1-ethyl-4-(trifluoromethyl)-1H-imidazol-2-yl)phenyl)ethyl)-6,7-dihydro-[1,2,4]triazolo[1,5-α]pyrimidin-5(4H)-one;

2-(4-cyclopropyl-6-methoxypyrimidin-5-yl)-4-(4-(1-(1-fluoropropan-2-yl)-4-(trifluoromethyl)-1H-imidazol-2-yl)benzyl)-6,7-dihydro-[1,2,4]triazolo[1,5-α]pyrimidin-5(4H)-one;

(S)-2-(4-cyclopropyl-6-methoxypyrimidin-5-yl)-4-(1-(3-fluoro-4-(1-methyl-4-(trifluoromethyl)-1H-imidazol-2-yl)phenyl)ethyl)-6,7-dihydro-[1,2,4]triazolo[1,5-α]pyrimidin-5(4H)-one;

(R)-2-(4-cyclopropyl-6-methoxypyrimidin-5-yl)-4-(1-(3-fluoro-4-(1-methyl-4-(trifluoromethyl)-1H-imidazol-2-yl)phenyl)ethyl)-6,7-dihydro-[1,2,4]triazolo[1,5-α]pyrimidin-5(4H)-one;

(S)-2-(4-(1-(2-(4-cyclopropyl-6-methoxypyrimidin-5-yl)-5-oxo-6,7-dihydro-[1,2,4]triazolo[1,5-α]pyrimidin-4(5H)-yl)ethyl)phenyl)-1-ethyl-1H-imidazole-4-carbonitrile;

(R)-2-(4-(1-(2-(4-cyclopropyl-6-methoxypyrimidin-5-yl)-5-oxo-6,7-dihydro-[1,2,4]triazolo[1,5-α]pyrimidin-4(5H)-yl)ethyl)phenyl)-1-ethyl-1H-imidazole-4-carbonitrile;

(S)-4-(1-(4-(1-cyclopropyl-4-(trifluoromethyl)-1H-imidazol-2-yl)phenyl)ethyl)-2-(4-cyclopropyl-6-methoxypyrimidin-5-yl)-6,7-dihydro-[1,2,4]triazolo[1,5-α]pyrimidin-5(4H)-one;

(R)-4-(1-(4-(1-cyclopropyl-4-(trifluoromethyl)-1H-imidazol-2-yl)phenyl)ethyl)-2-(4-cyclopropyl-6-methoxypyrimidin-5-yl)-6,7-dihydro-[1,2,4]triazolo[1,5-α]pyrimidin-5(4H)-one;

2-(4-cyclopropyl-6-methoxypyrimidin-5-yl)-4-(5-fluoro-4-(1-isopropyl-4-(trifluoromethyl)-1H-imidazol-2-yl)-2-methoxybenzyl)-6,7-dihydro-[1,2,4]triazolo[1,5-α]pyrimidin-5(4H)-one;

(S)-2-(4-cyclopropyl-6-methoxypyrimidin-5-yl)-4-(1-(3-fluoro-4-(5-methyl-3-(trifluoromethyl)-1H-pyrazol-1-yl)phenyl)ethyl)-6,7-dihydro-[1,2,4]triazolo[1,5-α]pyrimidin-5(4H)-one;

(R)-2-(4-cyclopropyl-6-methoxypyrimidin-5-yl)-4-(1-(3-fluoro-4-(5-methyl-3-(trifluoromethyl)-1H-pyrazol-1-yl)phenyl)ethyl)-6,7-dihydro-[1,2,4]triazolo[1,5-α]pyrimidin-5(4H)-one;

(S)-4-(1-(4-(5-bromo-1-methyl-4-(trifluoromethyl)-1H-imidazol-2-yl)phenyl)ethyl)-2-(4-cyclopropyl-6-methoxypyrimidin-5-yl)-6,7-dihydro-[1,2,4]triazolo[1,5-α]pyrimidin-5(4H)-one;

(R)-4-(1-(4-(5-bromo-1-methyl-4-(trifluoromethyl)-1H-imidazol-2-yl)phenyl)ethyl)-2-(4-cyclopropyl-6-methoxypyrimidin-5-yl)-6,7-dihydro-[1,2,4]triazolo[1,5-α]pyrimidin-5(4H)-one;

2-(2-cyclopropyl-4-methoxypyridin-3-yl)-4-(3-fluoro-4-(1-isopropyl-4-(trifluoromethyl)-1H-imidazol-2-yl)benzyl)-6,7-dihydro-[1,2,4]triazolo[1,5-α]pyrimidin-5(4H)-one;

(R)-2-(4-cyclopropyl-6-(difluoromethoxy)pyrimidin-5-yl)-4-(1-(4-(1-ethyl-4-(trifluoromethyl)-1H-imidazol-2-yl)phenyl)ethyl)-6,7-dihydro-[1,2,4]triazolo[1,5-α]pyrimidin-5(4H)-one;

(S)-2-(4-cyclopropyl-6-(difluoromethoxy)pyrimidin-5-yl)-4-(1-(4-(1-ethyl-4-(trifluoromethyl)-1H-imidazol-2-yl)phenyl)ethyl)-6,7-dihydro-[1,2,4]triazolo[1,5-α]pyrimidin-5(4H)-one;

2-(4-cyclopropyl-6-methoxypyrimidin-5-yl)-4-(3-fluoro-4-(1-isopropyl-4-(trifluoromethyl)-1H-imidazol-2-yl)-2-methoxybenzyl)-6,7-dihydro-[1,2,4]triazolo[1,5-α]pyrimidin-5(4H)-one;

(R)-4-(1-(3-chloro-4-(1-ethyl-4-(trifluoromethyl)-1H-imidazol-2-yl)phenyl)ethyl)-2-(4-cyclopropyl-6-methoxypyrimidin-5-yl)-6,7-dihydro-[1,2,4]triazolo[1,5-α]pyrimidin-5(4H)-one;

(S)-4-(1-(3-chloro-4-(1-ethyl-4-(trifluoromethyl)-1H-imidazol-2-yl)phenyl)ethyl)-2-(4-cyclopropyl-6-methoxypyrimidin-5-yl)-6,7-dihydro-[1,2,4]triazolo[1,5-α]pyrimidin-5(4H)-one;

2-(4-cyclopropyl-2-methoxypyridin-3-yl)-4-(3-fluoro-4-(1-isopropyl-4-(trifluoromethyl)-1H-imidazol-2-yl)benzyl)-6,7-dihydro-[1,2,4]triazolo[1,5-α]pyrimidin-5(4H)-one;

2-(4-cyclopropyl-6-methoxypyrimidin-5-yl)-4-(1-(4-(5-(2,2-difluoroethoxy)-3-(trifluoromethyl)-1H-pyrazol-1-yl)phenyl)ethyl)-6,7-dihydro-[1,2,4]triazolo[1,5-α]pyrimidin-5(4H)-one;

2-(4-chloro-1-isopropyl-1H-pyrazol-5-yl)-4-(1-(4-(5-(2,2-difluoroethoxy)-3-(trifluoromethyl)-1H-pyrazol-1-yl)phenyl)ethyl)-6,7-dihydro-[1,2,4]triazolo[1,5-α]pyrimidin-5(4H)-one;

2-(4-chloro-1-isopropyl-1H-pyrazol-5-yl)-4-(3-fluoro-4-(1-isopropyl-4-(trifluoromethyl)-1H-imidazol-2-yl)-2-methoxybenzyl)-6,7-dihydro-[1,2,4]triazolo[1,5-α]pyrimidin-5(4H)-one;

2-(4-cyclopropyl-6-methoxypyrimidin-5-yl)-4-(4-(1-ethyl-4-(trifluoromethyl)-1H-imidazol-2-yl)-3-fluoro-2-methoxybenzyl)-6,7-dihydro-[1,2,4]triazolo[1,5-α]pyrimidin-5(4H)-one;

2-(4-cyclopropyl-6-methoxypyrimidin-5-yl)-4-(1-(3-fluoro-4-(1-isopropyl-4-(trifluoromethyl)-1H-imidazol-2-yl)-2-methoxyphenyl)ethyl)-6,7-dihydro-[1,2,4]triazolo[1,5-α]pyrimidin-5(4H)-one;

2-(4-cyclopropyl-6-methoxypyrimidin-5-yl)-4-(1-(4-(3-(dimethylamino)pyridin-2-yl)phenyl)ethyl)-6,7-dihydro-[1,2,4]triazolo[1,5-α]pyrimidin-5(4H)-one;

2-(4-cyclopropyl-6-methoxypyrimidin-5-yl)-4-(1-(4-(3-isopropoxypyridin-2-yl)phenyl)ethyl)-6,7-dihydro-[1,2,4]triazolo[1,5-α]pyrimidin-5(4H)-one;

2-(1-isopropyl-4-methyl-1H-pyrazol-5-yl)-4-(4-(1-methyl-4-(trifluoromethyl)-1H-imidazol-2-yl)benzyl)-4,5,6,7-tetrahydro-[1,2,4]triazolo[1,5-α]pyrimidine;

4-(4-(1-isopropyl-4-(trifluoromethyl)-1H-imidazol-2-yl)benzyl)-2-(1-isopropyl-4-methyl-1H-pyrazol-5-yl)-4,5,6,7-tetrahydro-[1,2,4]triazolo[1,5-α]pyrimidine;

2-(4-chloro-1-isopropyl-1H-pyrazol-5-yl)-4-(4-(1-ethyl-4-(trifluoromethyl)-1H-imidazol-2-yl)benzyl)-5-oxo-4,5,6,7-tetrahydropyrazolo[1,5-α]pyrimidine-3-carbonitrile;

2-(4-chloro-1-isopropyl-1H-pyrazol-5-yl)-4-(4-(5-((methyl amino)methyl)-3-(trifluoromethyl)-1H-pyrazol-1-yl)benzyl)-6,7-dihydropyrazolo[1,5-α]pyrimidin-5(4H)-one;

2-(4-cyclopropyl-6-methoxypyrimidin-5-yl)-4-(4-(5-((methylamino)methyl)-3-(trifluoromethyl)-1H-pyrazol-1-yl)benzyl)-6,7-dihydropyrazolo[1,5-α]pyrimidin-5(4H)-one;

2-(4-chloro-1-isopropyl-1H-pyrazol-5-yl)-4-(4-(pyridin-2-ylmethoxy)benzyl)-6,7-dihydropyrazolo[1,5-α]pyrimidin-5(4H)-one;

2-(4-chloro-1-(1-hydroxypropan-2-yl)-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one;

2-(4-chloro-1-isopropyl-1H-pyrazol-5-yl)-4-(3-(2-hydroxyethoxy)-4-(1-isopropyl-4-(trifluoromethyl)-1H-imidazol-2-yl)benzyl)-6,7-dihydro-[1,2,4]triazolo[1,5-α]pyrimidin-5(4H)-one;

3-amino-2-(4-chloro-1-isopropyl-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one;

2-(4-chloro-1-isopropyl-1H-pyrazol-5-yl)-4-(4-(3-morpholino-1H-pyrazol-1-yl)benzyl)-6,7-dihydropyrazolo[1,5-α]pyrimidin-5(4H)-one;

2-(4-chloro-1-isopropyl-1H-pyrazol-5-yl)-6-hydroxy-4-(4-(1-methyl-4-(trifluoromethyl)-1H-imidazol-2-yl)benzyl)-6,7-dihydropyrazolo[1,5-α]pyrimidin-5(4H)-one;

2-(4-chloro-1-isopropyl-1H-pyrazol-5-yl)-6-hydroxy-6-methyl-4-(4-(1-methyl-4-(trifluoromethyl)-1H-imidazol-2-yl)benzyl)-6,7-dihydropyrazolo[1,5-α]pyrimidin-5(4H)-one;

6-amino-2-(4-chloro-1-isopropyl-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one;

N-(2-(4-chloro-1-isopropyl-1H-pyrazol-5-yl)-4-(4-(1-ethyl-4-(trifluoromethyl)-1H-imidazol-2-yl)benzyl)-5-oxo-4,5,6,7-tetrahydropyrazolo[1,5-α]pyrimidin-6-yl)acetamide;

2-(4-chloro-1-isopropyl-1H-pyrazol-5-yl)-6-(dimethyl amino)-4-(4-(1-ethyl-4-(trifluoromethyl)-1H-imidazol-2-yl)benzyl)-6,7-dihydropyrazolo[1,5-α]pyrimidin-5(4H)-one;

2-(4-chloro-1-isopropyl-1H-pyrazol-5-yl)-4-(4-(1-ethyl-4-(trifluoromethyl)-1H-imidazol-2-yl)benzyl)-6-(methylamino)-6,7-dihydropyrazolo[1,5-α]pyrimidin-5(4H)-one;

2-(4-chloro-1-isopropyl-1H-pyrazol-5-yl)-N-(4-(1-ethyl-4-(trifluoromethyl)-1H-imidazol-2-yl)phenyl)-4,5,6,7-tetrahydropyrazolo[1,5-α]pyridin-4-amine;

(R)-2-(4-chloro-1-isopropyl-1H-pyrazol-5-yl)-N-(4-(1-ethyl-4-(trifluoromethyl)-1H-imidazol-2-yl)phenyl)-4,5,6,7-tetrahydropyrazolo[1,5-α]pyridin-4-amine;

(S)-2-(4-chloro-1-isopropyl-1H-pyrazol-5-yl)-N-(4-(1-ethyl-4-(trifluoromethyl)-1H-imidazol-2-yl)phenyl)-4,5,6,7-tetrahydropyrazolo[1,5-α]pyridin-4-amine;

2-(4-cyclopropyl-6-methoxypyrimidin-5-yl)-N-(4-(1-ethyl-4-(trifluoromethyl)-1H-imidazol-2-yl)phenyl)-4,5,6,7-tetrahydropyrazolo[1,5-α]pyridin-4-amine;

2-(4-chloro-1-isopropyl-1H-pyrazol-5-yl)-N-(4-(1-ethyl-4-(trifluoromethyl)-1H-imidazol-2-yl)-3-fluorophenyl)-4,5,6,7-tetrahydropyrazolo[1,5-α]pyridin-4-amine;

(S)-2-(4-cyclopropyl-6-methoxypyrimidin-5-yl)-N-(4-(1-ethyl-4-(trifluoromethyl)-1H-imidazol-2-yl)phenyl)-4,5,6,7-tetrahydropyrazolo[1,5-α]pyridin-4-amine;

(R)-2-(4-cyclopropyl-6-methoxypyrimidin-5-yl)-N-(4-(1-ethyl-4-(trifluoromethyl)-1H-imidazol-2-yl)phenyl)-4,5,6,7-tetrahydropyrazolo[1,5-α]pyridin-4-amine;

2-(4-chloro-1-isopropyl-1H-pyrazol-5-yl)-N-(4-(5-methyl-3-(trifluoromethyl)-1H-pyrazol-1-yl)phenyl)-4,5,6,7-tetrahydropyrazolo[1,5-α]pyridin-4-amine;

2-(4-chloro-1-isopropyl-1H-pyrazol-5-yl)-N-(6-(1-ethyl-4-(trifluoromethyl)-1H-imidazol-2-yl)pyridin-3-yl)-4, 5 ,6,7-tetrahydropyrazolo[1,5-α]pyridin-4-amine;

2-(4-chloro-1-isopropyl-1H-pyrazol-5-yl)-N-(4-(1-ethyl-4-(trifluoromethyl)-1H-imidazol-2-yl)-3-methoxyphenyl)-4, 5, 6,7-tetrahydropyrazolo[1,5-α]pyridin-4-amine;

2-(4-((2-(4-chloro-1-isopropyl-1H-pyrazol-5-yl)-4, ,6,7-tetrahydropyrazolo[1, 5-α]pyridin-4-yl)amino)phenyl)-1-ethyl-1H-imidazole-4-carbonitrile;

2-(4-cyclopropyl-6-methoxypyrimidin-5-yl)-N-(4-(1-(1-fluoropropan-2-yl)-4-(trifluoromethyl)-1H-imidazol-2-yl)phenyl)-4,5,6,7-tetrahydropyrazolo[1,5-α]pyridin-4-amine;

2-(4-cyclopropyl-6-(difluoromethoxy)pyrimidin-5-yl)-N-(4-(1-(1-fluoropropan-2-yl)-4-(trifluoromethyl)-1H-imidazol-2-yl)phenyl)-4,5,6,7-tetrahydropyrazolo[1,5-α]pyridin-4-amine;

2-(4-cyclopropyl-6-(difluoromethoxy)pyrimidin-5-yl)-N-(4-(1-ethyl-4-(trifluoromethyl)-1H-imidazol-2-yl)phenyl)-4, 5, 6,7-tetrahydropyrazolo[1,5-α]pyridin-4-amine;

(R)-2-(4-cyclopropyl-6-methoxypyrimidin-5-yl)-N-(3-fluoro-4-(3-methoxypyridin-2-yl)phenyl)-4,5,6,7-tetrahydropyrazolo[1,5-α]pyridin-4-amine;

(S)-2-(4-cyclopropyl-6-methoxypyrimidin-5-yl)-N-(3-fluoro-4-(3-methoxypyridin-2-yl)phenyl)-4,5,6,7-tetrahydropyrazolo[1,5-α]pyridin-4-amine;

(R)-N-(3-chloro-4-(3-methoxypyridin-2-yl)phenyl)-2-(4-cyclopropyl-6-methoxypyrimidin-5-yl)-4, 5, 6,7-tetrahydropyrazolo[1,5-α]pyridin-4-amine;

(S)-N-(3-chloro-4-(3-methoxypyridin-2-yl)phenyl)-2-(4-cyclopropyl-6-methoxypyrimidin-5-yl)-4, 5, 6,7-tetrahydropyrazolo[1,5-α]pyridin-4-amine;

5-((2-(4-chloro-1-isopropyl-1H-pyrazol-5-yl)-4,5,6,7-tetrahydropyrazolo[1,5-α]pyridin-4-yl)amino)-2-(1-ethyl-4-(trifluoromethyl)-1H-imidazol-2-yl)benzonitrile;

2-(4-chloro-1-isopropyl-1H-pyrazol-5-yl)-N-(4-(1-ethyl-4-(trifluoromethyl)-1H-imidazol-2-yl)phenyl)-5,6,7,8-tetrahydro-[1,2,4]triazolo[1,5-α]pyridin-8-amine;

2-(4-chloro-1-isopropyl-1H-pyrazol-5-yl)-N-(4-(1-ethyl-4-(trifluoromethyl)-1H-imidazol-2-yl)-3-fluorophenyl)-5,6,7,8-tetrahydro-[1,2,4]triazolo[1,5-α]pyridin-8-amine;

2-(4-chloro-1-isopropyl-1H-pyrazol-5-yl)-N-(4-(1-ethyl-4-(trifluoromethyl)-1H-imidazol-2-yl)phenyl)-N-methyl-4,5,6,7-tetrahydropyrazolo[1,5-α]pyridin-4-amine;

2-(4-chloro-1-isopropyl-1H-pyrazol-5-yl)-N-(4-(1-ethyl-4-(trifluoromethyl)-1H-imidazol-2-yl)-3-fluorophenyl)-N-methyl-5,6,7,8-tetrahydro-[1,2,4]triazolo[1,5-α]pyridin-8-amine;

2-(4-chloro-1-isopropyl-1H-pyrazol-5-yl)-N-(4-(1-ethyl-4-(trifluoromethyl)-1H-imidazol-2-yl)phenyl)-N-methyl-5,6,7,8-tetrahydro-[1,2,4]triazolo[1,5-α]pyridin-8-amine;

2-(4-chloro-1-isopropyl-1H-pyrazol-5-yl)-4-(4-(1-ethyl-4-(trifluoromethyl)-1H-imidazol-2-yl)phenoxy)-4,5,6,7-tetrahydropyrazolo[1,5-α]pyridine;

2-(4-chloro-1-isopropyl-1H-pyrazol-5-yl)-4-(4-(1-ethyl-4-(trifluoromethyl)-1H-imidazol-2-yl)-3-fluorophenoxy)-4,5,6,7-tetrahydropyrazolo[1,5-α]pyridine;

(S)-2-(4-cyclopropyl-6-methoxypyrimidin-5-yl)-4-(4-(1-ethyl-4-(trifluoromethyl)-1H-imidazol-2-yl)-3-fluorophenoxy)-4,5,6,7-tetrahydropyrazolo[1,5-α]pyridine;

(R)-2-(4-cyclopropyl-6-methoxypyrimidin-5-yl)-4-(4-(1-ethyl-4-(trifluoromethyl)-1H-imidazol-2-yl)-3-fluorophenoxy)-4,5,6,7-tetrahydropyrazolo[1,5-α]pyridine;

2-(4-chloro-1-isopropyl-1H-pyrazol-5-yl)-4-(4-(1-ethyl-4-(trifluoromethyl)-1H-imidazol-2-yl)benzyl)-6,7-dihydro-4H-pyrazolo[5,1-c][1,4]oxazine;

5-(4((4-(1-ethyl-4-(trifluoromethyl)-1H-imidazol-2-yl)-3-fluorophenyl)amino)-4,5,6,7-tetrahydropyrazolo[1,5-α]pyridin-2-yl)-1-isopropyl-1H-pyrazole-4-carbonitrile;

2-(4-chloro-1-isopropyl-1H-pyrazol-5-yl)-4-(4-(1-ethyl-4-(trifluoromethyl)-1H-imidazol-2-yl)-3-fluorobenzyl)-4,5,6,7-tetrahydropyrazolo[1,5-α]pyrimidin-6-ol;

3-chloro-4-(4-(1-ethyl-4-(trifluoromethyl)-1H-imidazol-2-yl)benzyl)-2-(1-isopropyl-1H-pyrazol-5-yl)-6,7-dihydropyrazolo[1,5-α]pyrimidin-5(4H)-one;

3-chloro-N-(4-(1-ethyl-4-(trifluoromethyl)-1H-imidazol-2-yl)-3-fluorophenyl)-2-(1-isopropyl-1H-pyrazol-5-yl)-4,5,6,7-tetrahydropyrazolo[1,5-α]pyridin-4-amine;

2-(4-chloro-1-isopropyl-1H-pyrazol-5-yl)-4-(3-chloro-4-(3-(difluoromethoxy)pyridin-2-yl)benzyl)-6,7-dihydropyrazolo[1,5-α]pyrimidin-5(4H)-one;

2-(4-chloro-1-isopropyl-1H-pyrazol-5-yl)-4-(3-chloro-4-(6-(trifluoromethyl)pyrazin-2-yl)benzyl)-6,7-dihydropyrazolo[1,5-α]pyrimidin-5(4H)-one;

2-(4-chloro-1-isopropyl-1H-pyrazol-5-yl)-4-(3-chloro-4-(4-(trifluoromethyl)pyrimidin-2-yl)benzyl)-6,7-dihydropyrazolo[1,5-α]pyrimidin-5(4H)-one;

2-(4-chloro-1-isopropyl-1H-pyrazol-5-yl)-4-(3-chloro-4-(3-methoxypyrazin-2-yl)benzyl)-6,7-dihydropyrazolo[1,5-α]pyrimidin-5(4H)-one;

2-(4-chloro-1-isopropyl-1H-pyrazol-5-yl)-4-(3-chloro-4-(2-methylimidazo[1,2-α]pyrazin-8-yl)benzyl)-6,7-dihydropyrazolo[1,5-α]pyrimidin-5(4H)-one;

2-(4-chloro-1-isopropyl-1H-pyrazol-5-yl)-4-(3-chloro-4-(pyrrolo[2,1-f][1,2,4]triazin-4-yl)benzyl)-6,7-dihydropyrazolo[1,5-α]pyrimidin-5(4H)-one;

2-(4-chloro-1-isopropyl-1H-pyrazol-5-yl)-4-(3-chloro-4-(1,2-dimethyl-1H-imidazol-4-yl)benzyl)-6,7-dihydropyrazolo[1,5-α]pyrimidin-5(4H)-one;

2-(4-chloro-1-isopropyl-1H-pyrazol-5-yl)-4-(3-chloro-4-(4-methylpyridazin-3-yl)benzyl)-6,7-dihydropyrazolo[1,5-α]pyrimidin-5(4H)-one;

2-(4-chloro-1-isopropyl-1H-pyrazol-5-yl)-4-(3-chloro-4-(3-(trifluoromethoxy)pyridin-2-yl)benzyl)-6,7-dihydropyrazolo[1,5-α]pyrimidin-5(4H)-one;

2-(4-chloro-1-isopropyl-1H-pyrazol-5-yl)-4-(3-chloro-4-(5-methylpyrrolo[2,1-f][1,2,4]triazin-4-yl)benzyl)-6,7-dihydropyrazolo[1,5-α]pyrimidin-5(4H)-one;

2-(4-chloro-1-isopropyl-1H-pyrazol-5-yl)-4-(3-chloro-4-(1,3-dimethyl-1H-pyrazol-5-yl)benzyl)-6,7-dihydropyrazolo[1,5-α]pyrimidin-5(4H)-one;

2-(4-chloro-1-isopropyl-1H-pyrazol-5-yl)-4-(3-chloro-4-(3-fluoro-6-methylpyridin-2-yl)benzyl)-6,7-dihydropyrazolo[1,5-α]pyrimidin-5(4H)-one;

2-(4-chloro-1-isopropyl-1H-pyrazol-5-yl)-4-(3-chloro-4-(3-(trifluoromethyl)pyridin-2-yl)benzyl)-6,7-dihydropyrazolo[1,5-α]pyrimidin-5(4H)-one;

2-(4-chloro-1-isopropyl-1H-pyrazol-5-yl)-4-(3-chloro-4-(1,2-dimethyl-1H-imidazol-5-yl)benzyl)-6,7-dihydropyrazolo[1,5-α]pyrimidin-5(4H)-one;

2-(4-chloro-1-isopropyl-1H-pyrazol-5-yl)-4-(3-chloro-4-(5-methoxypyridin-2-yl)benzyl)-6,7-dihydropyrazolo[1,5-α]pyrimidin-5(4H)-one;

2-(4-chloro-1-isopropyl-1H-pyrazol-5-yl)-4-(3-chloro-4-(4-methoxypyrimidin-2-yl)benzyl)-6,7-dihydropyrazolo[1,5-α]pyrimidin-5(4H)-one;

3-(2-chloro-4-((2-(4-chloro-1-isopropyl-1H-pyrazol-5-yl)-5-oxo-6,7-dihydropyrazolo[1,5-α]pyrimidin-4(5H)-yl)methyl)phenyl)-1-methyl-1H-pyrazole-4-carbonitrile;

2-(4-chloro-1-isopropyl-1H-pyrazol-5-yl)-4-(3-chloro-4-(3-methoxypyridin-2-yl)benzyl)-6,7-dihydropyrazolo[1,5-α]pyrimidin-5(4H)-one;

2-(4-chloro-1-isopropyl-1H-pyrazol-5-yl)-4-(3-chloro-4-(1,3, 5-trimethyl-1H-pyrazol-4-yl)benzyl)-6,7-dihydropyrazolo[1,5-α]pyrimidin-5(4H)-one;

2-(4-chloro-1-isopropyl-1H-pyrazol-5-yl)-4-(3-chloro-4-(3-cyclopropoxypyridin-2-yl)benzyl)-6,7-dihydropyrazolo[1,5-α]pyrimidin-5(4H)-one;

2-(4-chloro-1-isopropyl-1H-pyrazol-5-yl)-4-(4-(3-isopropoxypyridin-2-yl)benzyl)-6,7-dihydropyrazolo[1,5-α]pyrimidin-5(4H)-one;

2-(4-chloro-1-isopropyl-1H-pyrazol-5-yl)-4-(3-chloro-4-(4-methoxy-1-methyl-1H-pyrazol-3-yl)benzyl)-6,7-dihydropyrazolo[1,5-α]pyrimidin-5(4H)-one;

2-(4-chloro-1-isopropyl-1H-pyrazol-5-yl)-4-(3-chloro-4-(4-methoxypyridazin-3-yl)benzyl)-6,7-dihydropyrazolo[1,5-α]pyrimidin-5(4H)-one;

2-(4-chloro-1-isopropyl-1H-pyrazol-5-yl)-4-(3-chloro-4-(3-methoxy-6-(trifluoromethyl)pyridin-2-yl)benzyl)-6,7-dihydropyrazolo[1,5-α]pyrimidin-5(4H)-one;

4-(3-chloro-4-(3-isopropoxypyridin-2-yl)benzyl)-2-(4-cyclopropyl-6-methoxypyrimidin-5-yl)-6,7-dihydro-[1,2,4]triazolo[1,5-α]pyrimidin-5(4H)-one;

2-(4-chloro-1-isopropyl-1H-pyrazol-5-yl)-4-(3-chloro-4-(3-morpholinopyridin-2-yl)benzyl)-6,7-dihydropyrazolo[1,5-α]pyrimidin-5(4H)-one;

2-(4-chloro-1-isopropyl-1H-pyrazol-5-yl)-4-(3-chloro-4-(4-fluoro-1-isopropyl-1H-imidazol-2-yl)benzyl)-6,7-dihydropyrazolo[1,5-α]pyrimidin-5(4H)-one;

2-(4-chloro-1-isopropyl-1H-pyrazol-5-yl)-4-(3-chloro-4-(3-methoxy-4-methylpyridin-2-yl)benzyl)-6,7-dihydropyrazolo[1,5-α]pyrimidin-5(4H)-one;

2-(4-chloro-1-isopropyl-1H-pyrazol-5-yl)-4-(3-chloro-4-(4-methyl-3,4-dihydro-2H-pyrido[4,3-b][1,4]oxazin-5-yl)benzyl)-6,7-dihydropyrazolo[1,5-α]pyrimidin-5(4H)-one;

2-(4-chloro-1-isopropyl-1H-pyrazol-5-yl)-4-(3-chloro-4-(3-(methoxymethyl)pyridin-2-yl)benzyl)-6,7-dihydropyrazolo[1,5-α]pyrimidin-5(4H)-one;

2-(4-chloro-1-isopropyl-1H-pyrazol-5-yl)-4-(3-chloro-4-(3-methoxy-5-methylpyridin-2-yl)benzyl)-6,7-dihydropyrazolo[1,5-α]pyrimidin-5(4H)-one;

2-(4-chloro-1-isopropyl-1H-pyrazol-5-yl)-4-(3-chloro-4-(3-isopropoxypyridin-2-yl)benzyl)-6,7-dihydro-[1,2,4]triazolo[1,5-α]pyrimidin-5(4H)-one;

2-(4-chloro-1-isopropyl-1H-pyrazol-5-yl)-4-(3-chloro-4-(1-isopropyl-1H-imidazol-2-yl)benzyl)-6,7-dihydropyrazolo[1,5-α]pyrimidin-5(4H)-one;

2-(4-chloro-1-isopropyl-1H-pyrazol-5-yl)-4-(4-(3-isopropoxypyridin-2-yl)benzyl)-6,7-dihydro-[1,2,4]triazolo[1,5-α]pyrimidin-5(4H)-one;

2-(4-chloro-1-isopropyl-1H-pyrazol-5-yl)-4-(4-(3-methoxypyridin-2-yl)benzyl)-6,7-dihydropyrazolo[1,5-α]pyrimidin-5(4H)-one;

4-(3-chloro-4-(3-methoxypyridin-2-yl)benzyl)-2-(4-cyclopropyl-6-methoxypyrimidin-5-yl)-6,7-dihydro-[1,2,4]triazolo[1,5-α]pyrimidin-5(4H)-one;

2-(4-chloro-1-isopropyl-1H-pyrazol-5-yl)-4-(3-chloro-4-(3-ethoxypyridin-2-yl)benzyl)-6,7-dihydropyrazolo[1,5-α]pyrimidin-5(4H)-one;

2-(4-cyclopropyl-6-methoxypyrimidin-5-yl)-4-(3-fluoro-4-(3-methoxypyridin-2-yl)benzyl)-6,7-dihydro-[1,2,4]triazolo[1,5-α]pyrimidin-5(4H)-one;

2-(4-chloro-1-isopropyl-1H-pyrazol-5-yl)-4-(3-chloro-4-(3-methoxy-6-methylpyridin-2-yl)benzyl)-6,7-dihydropyrazolo[1,5-α]pyrimidin-5(4H)-one;

2-(4-chloro-1-isopropyl-1H-pyrazol-5-yl)-4-(3-chloro-4-(3-isopropoxypyridin-2-yl)benzyl)-6,7-dihydropyrazolo[1,5-α]pyrimidin-5(4H)-one;

2-(4-chloro-1-isopropyl-1H-pyrazol-5-yl)-4-(4-(3-chloropyridin-2-yl)-3-methoxybenzyl)-6,7-dihydropyrazolo[1,5-α]pyrimidin-5(4H)-one;

2-(4-chloro-1-isopropyl-1H-pyrazol-5-yl)-4-(3-chloro-4-(3-(dimethylamino)pyridin-2-yl)benzyl)-6,7-dihydropyrazolo[1,5-α]pyrimidin-5(4H)-one;

4-(3-chloro-4-(3-methoxypyridin-2-yl)benzyl)-2-(4-cyclopropyl-6-methoxypyrimidin-5-yl)-6,7-dihydropyrazolo[1,5-α]pyrimidin-5(4H)-one;

2-(4-chloro-1-isopropyl-1H-pyrazol-5-yl)-4-(3-chloro-4-(6-(ethylamino)pyridazin-3-yl)benzyl)-6,7-dihydropyrazolo[1,5-α]pyrimidin-5(4H)-one;

2-(4-chloro-1-isopropyl-1H-pyrazol-5-yl)-4-(3-chloro-4-(1-isopropyl-1H-imidazol-4-yl)benzyl)-6,7-dihydropyrazolo[1,5-α]pyrimidin-5(4H)-one;

2-(4-chloro-1-isopropyl-1H-pyrazol-5-yl)-4-(3-chloro-4-(3-fluoro-6-(trifluoromethyl)pyridin-2-yl)benzyl)-6,7-dihydropyrazolo[1,5-α]pyrimidin-5(4H)-one;

2-(4-cyclopropyl-6-methoxypyrimidin-5-yl)-4-(3-fluoro-4-(3-methoxypyridin-2-yl)benzyl)-6,7-dihydropyrazolo[1,5-α]pyrimidin-5(4H)-one;

2-(4-chloro-1-isopropyl-1H-pyrazol-5-yl)-4-(3-chloro-4-(5-methoxypyrimidin-4-yl)benzyl)-6,7-dihydropyrazolo[1,5-α]pyrimidin-5(4H)-one;

2-(4-chloro-1-isopropyl-1H-pyrazol-5-yl)-4-(3-chloro-4-(6-methoxypyridazin-3-yl)benzyl)-6,7-dihydropyrazolo[1,5-α]pyrimidin-5(4H)-one;

3-(2-chloro-4-((2-(4-chloro-1-isopropyl-1H-pyrazol-5-yl)-5-oxo-6,7-dihydropyrazolo[1,5-α]pyrimidin-4(5H)-yl)methyl)phenyl)-N,N-dimethyl-1H-pyrazole-1-sulfonamide;

2-(4-chloro-1-isopropyl-1H-pyrazol-5-yl)-4-(4-(3-(dimethyl amino)pyridin-2-yl)benzyl)-6,7-dihydropyrazolo[1,5-α]pyrimidin-5(4H)-one;

2-(4-chloro-1-isopropyl-1H-pyrazol-5-yl)-4-(4-(3-(2,2-difluoroethoxy)pyridin-2-yl)benzyl)-6,7-dihydro-[1,2,4]triazolo[1,5-α]pyrimidin-5(4H)-one;

2-(4-chloro-1-((R)-1-fluoropropan-2-yl)-1H-pyrazol-5-yl)-N-(4-(1-ethyl-4-(trifluoromethyl)-1H-imidazol-2-yl)phenyl)-4,5,6,7-tetrahydropyrazolo[1,5-α]pyridin-4-amine;

2-(4-chloro-1-((S)-1-fluoropropan-2-yl)-1H-pyrazol-5-yl)-N-(4-(1-ethyl-4-(trifluoromethyl)-1H-imidazol-2-yl)phenyl)-4,5,6,7-tetrahydropyrazolo[1,5-α]pyridin-4-amine;

(S)-5-(4-(1-(4-(1-ethyl-4-(trifluoromethyl)-1H-imidazol-2-yl)-3-fluorophenyl)ethyl)-5-oxo-4,5,6,7-tetrahydropyrazolo[1,5-α]pyrimidin-2-yl)-1-isopropyl-1H-pyrazole-4-carbonitrile;

(R)-5-(4-(1-(4-(1-ethyl-4-(trifluoromethyl)-1H-imidazol-2-yl)-3-fluorophenyl)ethyl)-5-oxo-4,5,6,7-tetrahydropyrazolo[1,5-α]pyrimidin-2-yl)-1-isopropyl-1H-pyrazole-4-carbonitrile;

2-(4-cyclopropyl-6-methoxypyrimidin-5-yl)-4-(4-(1-ethyl-4-(trifluoromethyl)-1H-imidazol-2-yl)-3-fluorobenzyl)-6,6-dimethyl-4,5,6,7-tetrahydropyrazolo[1,5-α]pyrimidine;

2-(4-(difluoromethyl)-1-isopropyl-1H-pyrazol-5-yl)-4-(4-(1-ethyl-4-(trifluoromethyl)-1H-imidazol-2-yl)-3-fluorobenzyl)-6,7-dihydropyrazolo[1,5-α]pyrimidin-5(4H)-one;

2-(4-(difluoromethoxy)-1-isopropyl-1H-pyrazol-5-yl)-4-(4-(1-ethyl-4-(trifluoromethyl)-1H-imidazol-2-yl)-3-fluorobenzyl)-6,7-dihydropyrazolo[1,5-α]pyrimidin-5(4H)-one;

2-((2-(4-chloro-1-isopropyl-1H-pyrazol-5-yl)-4,5,6,7-tetrahydropyrazolo[1,5-α]pyridin-4-yl)(4-(1-ethyl-4-(trifluoromethyl)-1H-imidazol-2-yl)-3-fluorophenyl)amino)-ethan-1-ol;

2-(4-cyclopropyl-6-methoxypyrimidin-5-yl)-4-(4-(1-ethyl-4-(trifluoromethyl)-1H-imidazol-2-yl)benzyl)pyrazolo[1,5-α]pyrimidin-5(4H)-one;

(R)-2-(4-cyclopropyl-6-methoxypyrimidin-5-yl)-4-(1-(4-(1-ethyl-4-(trifluoromethyl)-1H-imidazol-2-yl)-3-fluorophenyl)ethyl)pyrazolo[1,5-α]pyrimidin-5(4H)-one;

(S)-2-(4-cyclopropyl-6-methoxypyrimidin-5-yl)-4-(1-(4-(1-ethyl-4-(trifluoromethyl)-1H-imidazol-2-yl)-3-fluorophenyl)ethyl)pyrazolo[1,5-α]pyrimidin-5(4H)-one;

(R)-2-(4-cyclopropyl-6-methoxypyrimidin-5-yl)-4-(1-(4-(1-ethyl-4-(trifluoromethyl)-1H-imidazol-2-yl)phenyl)ethyl)pyrazolo[1,5-α]pyrimidin-5(4H)-one;

(S)-2-(4-cyclopropyl-6-methoxypyrimidin-5-yl)-4-(1-(4-(1-ethyl-4-(trifluoromethyl)-1H-imidazol-2-yl)phenyl)ethyl)pyrazolo[1,5-α]pyrimidin-5(4H)-one;

2-(4-cyclopropyl-6-methoxypyrimidin-5-yl)-4-(4-(1-ethyl-4-(trifluoromethyl)-1H-imidazol-2-yl)benzyl)-[1,2,4]triazolo[1,5-α]pyrimidin-5(4H)-one;

(R)-2-(4-cyclopropyl-6-methoxypyrimidin-5-yl)-4-(1-(4-(1-ethyl-4-(trifluoromethyl)-1H-imidazol-2-yl)phenyl)ethyl)-[1,2,4]triazolo[1,5-α]pyrimidin-5(4H)-one;

(S)-2-(4-cyclopropyl-6-methoxypyrimidin-5-yl)-4-(1-(4-(1-ethyl-4-(trifluoromethyl)-1H-imidazol-2-yl)phenyl)ethyl)-[1,2,4]triazolo[1,5-α]pyrimidin-5(4H)-one;

2-(4-cyclopropyl-6-methoxypyrimidin-5-yl)-4-(1-(4-(1-ethyl-4-(trifluoromethyl)-1H-imidazol-2-yl)phenyl)ethyl)-[1,2,4]triazolo[1,5-α]pyrimidin-5(4H)-one;

(R)-2-(4-cyclopropyl-6-methoxypyrimidin-5-yl)-4-(1-(4-(1-ethyl-4-(trifluoromethyl)-1H-imidazol-2-yl)-3-fluorophenyl)ethyl)-[1,2,4]triazolo[1,5-α]pyrimidin-5(4H)-one;

(S)-2-(4-cyclopropyl-6-methoxypyrimidin-5-yl)-4-(1-(4-(1-ethyl-4-(trifluoromethyl)-1H-imidazol-2-yl)-3-fluorophenyl)ethyl)-[1,2,4]triazolo[1,5-α]pyrimidin-5(4H)-one;

2-(4-chloro-1-isopropyl-1H-pyrazol-5-yl)-4-(4-(1-ethyl-4-(trifluoromethyl)-1H-imidazol-2-yl)-3-fluorobenzyl)-[1,2,4]triazolo[1,5-α]pyrimidin-5(4H)-one;

2-(4-cyclopropyl-6-(difluoromethoxy)pyrimidin-5-yl)-4-(4-(1-isopropyl-4-(trifluoromethyl)-1H-imidazol-2-yl)benzyl)-[1,2,4]triazolo[1,5-α]pyrimidin-5(4H)-one;

2-(4-cyclopropyl-6-(difluoromethoxy)pyrimidin-5-yl)-4-(4-(1-ethyl-4-(trifluoromethyl)-1H-imidazol-2-yl)benzyl)-[1,2,4]triazolo[1,5-α]pyrimidin-5(4H)-one;

2-(4-cyclopropyl-6-methoxypyrimidin-5-yl)-4-(3-fluoro-4-(1-isopropyl-4-(trifluoromethyl)-1H-imidazol-2-yl)-2-methoxybenzyl)-[1,2,4]triazolo[1,5-α]pyrimidin-5(4H)-one;

2-(4-cyclopropyl-6-methoxypyrimidin-5-yl)-4-(4-(1-ethyl-4-(trifluoromethyl)-1H-imidazol-2-yl)-3-fluorobenzyl)-6-methyl-6,7-dihydro-[1,2,4]triazolo[1,5-α]pyrimidin-5(4H)-one;

(R)-2-(4-cyclopropyl-6-methoxypyrimidin-5-yl)-4-(4-(1-ethyl-4-(trifluoromethyl)-1H-imidazol-2-yl)-3-fluorobenzyl)-6-methyl-6,7-dihydro-[1,2,4]triazolo[1,5-α]pyrimidin-5(4H)-one;

(S)-2-(4-cyclopropyl-6-methoxypyrimidin-5-yl)-4-(4-(1-ethyl-4-(trifluoromethyl)-1H-imidazol-2-yl)-3-fluorobenzyl)-6-methyl-6,7-dihydro-[1,2,4]triazolo[1,5-α]pyrimidin-5(4H)-one;

2-(4-chloro-1-isopropyl-1H-pyrazol-5-yl)-4-(4-(1-ethyl-4-(trifluoromethyl)-1H-imidazol-2-yl)-3-fluorobenzyl)-6-methyl-6,7-dihydro-[1,2,4]triazolo[1,5-α]pyrimidin-5(4H)-one;

2-(4-cyclopropyl-6-(difluoromethoxy)pyrimidin-5-yl)-4-(4-(1-ethyl-4-(trifluoromethyl)-1H-imidazol-2-yl)-3-fluorobenzyl)-6-methyl-6,7-dihydro-[1,2,4]triazolo[1,5-α]pyrimidin-5(4H)-one;

2-(4-chloro-1-isopropyl-1H-pyrazol-5-yl)-N-(4-(1-ethyl-4-(trifluoromethyl)-1H-imidazol-2-yl)phenyl)-5,5-dimethyl-4,5,6,7-tetrahydropyrazolo[1,5-α]pyridin-4-amine;

2-(4-chloro-1-isopropyl-1H-pyrazol-5-yl)-4-(4-(1-ethyl-4-(trifluoromethyl)-1H-imidazol-2-yl)-3-fluorobenzyl)-4,5,6,7-tetrahydropyrazolo[1,5-α]pyrimidin-6-amine;

2-(4-chloro-1-isopropyl-1H-pyrazol-5-yl)-4-((5-(1-ethyl-4-(trifluoromethyl)-1H-imidazol-2-yl)pyrimidin-2-yl)methyl)-6,7-dihydropyrazolo[1,5-α]pyrimidin-5(4H)-one;

2-(4-chloro-1-isopropyl-1H-pyrazol-5-yl)-4-(4-(1-ethyl-4-(trifluoromethyl)-1H-imidazol-2-yl)benzyl)-4,5,6,7-tetrahydropyrazolo[1,5-α]pyrazine;

2-(4-chloro-1-isopropyl-1H-pyrazol-5-yl)-4-(4-(1-ethyl-4-(trifluoromethyl)-1H-imidazol-2-yl)benzyl)-5-methyl-4,5,6,7-tetrahydropyrazolo[1,5-α]pyrazine;

2-(4-cyclopropyl-6-methoxypyrimidin-5-yl)-4-(4-(1-ethyl-4-(trifluoromethyl)-1H-imidazol-2-yl)benzyl)-4,5,6,7-tetrahydropyrazolo[1,5-α]pyrazine;

2-(4-cyclopropyl-6-methoxypyrimidin-5-yl)-4-(4-(1-ethyl-4-(trifluoromethyl)-1H-imidazol-2-yl)benzyl)-5-methyl-4,5,6,7-tetrahydropyrazolo[1,5-α]pyrazine;

(R)-2-(4-cyclopropyl-6-methoxypyrimidin-5-yl)-4-(1-(4-(1-isopropyl-4-(trifluoromethyl)-1H-imidazol-2-yl)phenyl)ethyl)-6,7-dihydropyrazolo[1,5-α]pyrimidin-5(4H)-one;

2-(4-cyclopropyl-6-(difluoromethoxy)pyrimidin-5-yl)-4-(4-(1-ethyl-4-(trifluoromethyl)-1H-imidazol-2-yl)benzyl)-6,7-dihydro-[1,2,4]triazolo[1,5-α]pyrimidin-5(4H)-one;

2-(4-cyclopropyl-6-(difluoromethoxy)pyrimidin-5-yl)-4-(3-fluoro-4-(1-isopropyl-4-(trifluoromethyl)-1H-imidazol-2-yl)-2-methoxybenzyl)-6,7-dihydro-[1,2,4]triazolo[1,5-α]pyrimidin-5(4H)-one;

2-(4-cyclopropyl-6-(difluoromethoxy)pyrimidin-5-yl)-4-(4-(1-(1-fluoropropan-2-yl)-4-(trifluoromethyl)-1H-imidazol-2-yl)benzyl)-6,7-dihydro-[1,2,4]triazolo[1,5-α]pyrimidin-5(4H)-one;

2-(4-cyclopropyl-6-(difluoromethoxy)pyrimidin-5-yl)-4-(4-(1-(2-fluoroethyl)-4-(trifluoromethyl)-1H-imidazol-2-yl)benzyl)-6,7-dihydro-[1,2,4]triazolo[1,5-α]pyrimidin-5(4H)-one;

2-(4-cyclopropyl-6-(difluoromethoxy)pyrimidin-5-yl)-4-(4-(1-methyl-4-(trifluoromethyl)-1H-imidazol-2-yl)benzyl)-6,7-dihydro-[1,2,4]triazolo[1,5-α]pyrimidin-5(4H)-one;

2-(4-cyclopropyl-6-(difluoromethoxy)pyrimidin-5-yl)-4-(4-(1-isopropyl-4-(trifluoromethyl)-1H-imidazol-2-yl)benzyl)-6,7-dihydro-[1,2,4]triazolo[1,5-α]pyrimidin-5(4H)-one;

2-(4-cyclopropyl-6-(difluoromethoxy)pyrimidin-5-yl)-4-(4-(1-ethyl-4-(trifluoromethyl)-1H-imidazol-2-yl)-3-fluorobenzyl)-6,7-dihydro-[1,2,4]triazolo[1,5-α]pyrimidin-5(4H)-one;

2-(4-chloro-1-isopropyl-1H-pyrazol-5-yl)-4-(2,2,2-trifluoro-1-(3-fluoro-4-(1-isopropyl-4-(trifluoromethyl)-1H-imidazol-2-yl)phenyl)ethyl)-4,5,6,7-tetrahydropyrazolo[1,5-α]pyrimidine;

2-(4-cyclopropyl-6-methoxypyrimidin-5-yl)-4-(1-(4-(5-methoxy-3-(trifluoromethyl)-1H-pyrazol-1-yl)phenyl)ethyl)-6,7-dihydro-[1,2,4]triazolo[1,5-α]pyrimidin-5(4H)-one;

2-(4-cyclopropyl-2-(difluoromethoxy)pyridin-3-yl)-4-(4-(1-ethyl-4-(trifluoromethyl)-1H-imidazol-2-yl)benzyl)-6,7-dihydro-[1,2,4]triazolo[1,5-α]pyrimidin-5(4H)-one;

2-(4-cyclopropyl-6-(difluoromethoxy)pyrimidin-5-yl)-4-(4-(1-(propan-2-yl-d₇)-4-(trifluoromethyl)-1H-imidazol-2-yl)benzyl)-6,7-dihydro-[1,2,4]triazolo[1,5-α]pyrimidin-5(4H)-one;

2-(4-cyclopropyl-6-(difluoromethoxy)pyrimidin-5-yl)-4-(4-(1-(ethyl-d₅)-4-(trifluoromethyl)-1H-imidazol-2-yl)benzyl)-6,7-dihydro-[1,2,4]triazolo[1,5-α]pyrimidin-5(4H)-one;

2-(4-(difluoromethoxy)-6-methoxypyrimidin-5-yl)-4-(4-(1-ethyl-4-(trifluoromethyl)-1H-imidazol-2-yl)benzyl)-6,7-dihydro-[1,2,4]triazolo[1,5-α]pyrimidin-5(4H)-one;

2-(4-cyclopropyl-6-(difluoromethoxy)pyrimidin-5-yl)-4-(3-fluoro-4-(1-methyl-4-(trifluoromethyl)-1H-imidazol-2-yl)benzyl)-6,7-dihydro-[1,2,4]triazolo[1,5-α]pyrimidin-5(4H)-one;

2-(4-cyclopropyl-6-methoxypyrimidin-5-yl)-4-(1-(4-(1-methyl-4-(trifluoromethyl)-1H-imidazol-2-yl)phenyl)ethyl)-6,7-dihydro-[1,2,4]triazolo[1,5-α]pyrimidin-5(4H)-one;

2-(4-cyclopropyl-6-methoxypyrimidin-5-yl)-4-(1-(4-(1-(2-fluoroethyl)-4-(trifluoromethyl)-1H-imidazol-2-yl)phenyl)ethyl)-6,7-dihydro-[1,2,4]triazolo[1,5-α]pyrimidin-5(4H)-one;

2-(4-cyclopropyl-6-methoxypyrimidin-5-yl)-4-(1-(4-(1-ethyl-4-(trifluoromethyl)-1H-imidazol-2-yl)-3-fluoro-2-methoxyphenyl)ethyl)-6,7-dihydro-[1,2,4]triazolo[1,5-α]pyrimidin-5(4H)-one;

(S)-2-(4-cyclopropyl-6-methoxypyrimidin-5-yl)-4-(4-(1-ethyl-4-(trifluoromethyl)-1H-imidazol-2-yl)benzyl)-6-methyl-6,7-dihydro-[1,2,4]triazolo[1,5-α]pyrimidin-5(4H)-one;

(R)-2-(4-cyclopropyl-6-methoxypyrimidin-5-yl)-4-(4-(1-ethyl-4-(trifluoromethyl)-1H-imidazol-2-yl)benzyl)-6-methyl-6,7-dihydro-[1,2,4]triazolo[1,5-α]pyrimidin-5(4H)-one;

2-(4-cyclopropyl-6-methoxypyrimidin-5-yl)-4-(4-(1-(1-fluoropropan-2-yl)-4-(trifluoromethyl)-1H-imidazol-2-yl)benzyl)-6-methyl-6,7-dihydro-[1,2,4]triazolo[1,5-α]pyrimidin-5(4H)-one;

2-(4-cyclopropyl-6-methoxypyrimidin-5-yl)-4-(1-(4-(1-ethyl-4-(trifluoromethyl)-1H-imidazol-2-yl)phenyl)ethyl)-6-methyl-6,7-dihydro-[1,2,4]triazolo[1,5-α]pyrimidin-5(4H)-one; 2-(4-cyclopropyl-6-(difluoromethoxy)pyrimidin-5-yl)-4-(3-fluoro-4-(1-methyl-4-(trifluoromethyl)-1H-imidazol-2-yl)benzyl)-[1,2,4]triazolo[1,5-α]pyrimidin-5(4H)-one;

2-(4-cyclopropyl-6-methoxypyrimidin-5-yl)-4-(3-fluoro-4-(1-methyl-4-(trifluoromethyl)-1H-imidazol-2-yl)benzyl)-[1,2,4]triazolo[1,5-α]pyrimidin-5(4H)-one;

2-(4-cyclopropyl-6-methoxypyrimidin-5-yl)-N-(4-(1-(2-fluoroethyl)-4-(trifluoromethyl)-1H-imidazol-2-yl)phenyl)-4,5,6,7-tetrahydropyrazolo[1,5-α]pyridin-4-amine;

2-(4-cyclopropyl-6-methoxypyrimidin-5-yl)-N-(3-fluoro-4-(1-methyl-4-(trifluoromethyl)-1H-imidazol-2-yl)phenyl)-4,5,6,7-tetrahydropyrazolo[1,5-α]pyridin-4-amine;

2-(4-cyclopropyl-6-methoxypyrimidin-5-yl)-N-(4-(1-(ethyl-d₅)-4-(trifluoromethyl)-1H-imidazol-2-yl)phenyl)-4,5,6,7-tetrahydropyrazolo[1,5-α]pyridin-4-amine;

2-(4-chloro-1-isopropyl-1H-pyrazol-5-yl)-N-(4-(1-(1-fluoropropan-2-yl)-4-(trifluoromethyl)-1H-imidazol-2-yl)phenyl)-4,5,6,7-tetrahydropyrazolo[1,5-α]pyridin-4-amine;

2-(4-cyclopropyl-6-methoxypyrimidin-5-yl)-4-(4-(1-ethyl-4-(trifluoromethyl)-1H-imidazol-2-yl)benzyl)-6-methyl-[1,2,4]triazolo[1,5-α]pyrimidin-5(4H)-one;

2-(4-cyclopropyl-6-(difluoromethoxy)pyrimidin-5-yl)-4-(4-(1-ethyl-4-(trifluoromethyl)-1H-imidazol-2-yl)benzyl)-6-methyl-[1,2,4]triazolo[1,5-α]pyrimidin-5(4H)-one; and

(4S)-2-(4-cyclopropyl-6-methoxypyrimidin-5-yl)-4-(4-(1-(1-fluoropropan-2-yl)-4-(trifluoromethyl)-1H-imidazol-2-yl)phenoxy)-4,5,6,7-tetrahydropyrazolo[1,5-α]pyridine, or a pharmaceutically acceptable salt or solvate, e.g., hydrate, of any of the above.

Definitions

For the purpose of the present disclosure, the term “alkyl” as used by itself or as part of another group refers to a straight- or branched-chain aliphatic hydrocarbon containing one to twelve carbon atoms (i.e., C₁₋₁₂ alkyl) or the number of carbon atoms designated (i.e., a Ci alkyl such as methyl, a C₂ alkyl such as ethyl, a C₃ alkyl such as propyl or isopropyl, etc.). The alkyl group can be suitably chosen from a straight chain C₁₋₁₀ alkyl group, a branched chain C₃₋₁₀ alkyl group, a straight chain C₁₋₆ alkyl group, a branched chain C₃₋₆ alkyl group, a straight chain C₁₋₄ alkyl group, a branched chain C₃₋₄ alkyl group, a straight or branched chain C₃₋₄ alkyl group. The alkyl group can be partially or completely deuterated, i.e., one or more hydrogen atoms of the alkyl group are replaced with deuterium atoms. Non-limiting exemplary C₁₋₁₀ alkyl groups include methyl (including —CD3), ethyl, propyl, isopropyl, butyl, sec-butyl, tent-butyl, iso-butyl, 3-pentyl, hexyl, heptyl, octyl, nonyl, and decyl. Non-limiting exemplary C₁₋₄ alkyl groups include methyl, ethyl, propyl, isopropyl, butyl, sec-butyl, tent-butyl, and iso-butyl.

For the purpose of the present disclosure, the term “optionally substituted alkyl” as used by itself or as part of another group means that the alkyl as defined above is either unsubstituted or substituted with one, two, or three substituents independently chosen from halo, nitro, cyano, hydroxy, amino, alkylamino, dialkylamino, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkyl sulfonyl, aryl sulfonyl, carboxy, carboxyalkyl, alkoxyalkyl, (amino)alkyl, hydroxyalkylamino, (alkylamino)alkyl, (dialkylamino)alkyl, (cyano)alkyl, (carboxamido)alkyl, mercaptoalkyl, (heterocyclo)alkyl, or (heteroaryl)alkyl. The alkyl can be an optionally substituted C₁₋₄ alkyl. The optionally substituted alkyl can be substituted with two substituents, or one substituent. Non-limiting exemplary optionally substituted alkyl groups include —CH₂CH₂NO₂, —CH₂CH₂CO₂H, —CH₂CH₂SO₂CH₃, —CH₂CH₂COPh, and —CH₂C₆H₁₁.

For the purpose of the present disclosure, the term “alkylene” or “alkylenyl” refers to a divalent alkyl radical. Any of the above mentioned monovalent alkyl groups may be an alkylene by abstraction of a second hydrogen atom from the alkyl. The alkylene group may also be a C₁-C₆ alkylene or a C₁-C₄ alkylene. Non-limiting exemplary alkylene groups include, —CH₂—, —CH(CH₃)—, —C(CH₃)₂—, —CH₂CH₂—, —CH₂CH(CH₃)—, —CH₂C(CH₃)₂—, —CH₂CH₂CH₂—, and —CH₂CH₂CH₂CH₂—.

For the purpose of the present disclosure, the term “cycloalkyl” as used by itself or as part of another group refers to saturated and partially unsaturated (containing one or two double bonds) cyclic aliphatic hydrocarbons containing one to three rings having from three to twelve carbon atoms (i.e., C₃₋₁₂ cycloalkyl) or the number of carbons designated. The cycloalkyl group can have two rings, or one ring. The cycloalkyl group can be chosen from a C₃₋₈ cycloalkyl group and a C₃₋₆ cycloalkyl group. The cycloalkyl group can contain one or more carbon-to-carbon double bonds or one carbon-to-carbon double bond. Non-limiting exemplary cycloalkyl groups include cyclopropyl, cyclobutyl, cyclopentyl, cyclohexyl, cycloheptyl, cyclooctyl, norbornyl, decalin, adamantyl, cyclohexenyl, and spiro[3.3]heptane.

For the purpose of the present disclosure, the term “optionally substituted cycloalkyl” as used by itself or as part of another group means that the cycloalkyl as defined above is either unsubstituted or substituted with one, two, or three substituents independently chosen from halo, nitro, cyano, hydroxy, amino, alkylamino, dialkylamino, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkyl sulfonyl, aryl sulfonyl, carboxy, carboxyalkyl, alkyl, optionally substituted cycloalkyl, alkenyl, alkynyl, optionally substituted aryl, optionally substituted heteroaryl, optionally substituted heterocyclo, alkoxyalkyl, (amino)alkyl, hydroxyalkylamino, (alkylamino)alkyl, (dialkylamino)alkyl, (cyano)alkyl, (carboxamido)alkyl, mercaptoalkyl, (heterocyclo)alkyl, or (heteroaryl)alkyl. The optionally substituted cycloalkyl can be substituted with two substituents or one substituent.

For the purpose of the present disclosure, the term “cycloalkyloxy” as used by itself or as part of another group refers to a cycloalkyl group attached to a terminal oxygen atom. A non-limiting exemplary of a cycloalkyloxy group is:

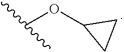 [SMILES: *OC1CC1]

For the purpose of the present disclosure, the term “alkenyl” as used by itself or as part of another group refers to an alkyl group as defined above containing one, two or three carbon-to-carbon double bonds. The alkenyl group can be chosen from a C₂₋₆ alkenyl group and a C₂₋₄ alkenyl group. Non-limiting exemplary alkenyl groups include ethenyl, propenyl, isopropenyl, butenyl, sec-butenyl, pentenyl, and hexenyl.

For the purpose of the present disclosure, the term “optionally substituted alkenyl” as used herein by itself or as part of another group means the alkenyl as defined above is either unsubstituted or substituted with one, two or three substituents independently chosen from halo, nitro, cyano, hydroxy, amino, alkylamino, dialkylamino, haloalkyl, hydroxyalkyl, alkoxy, haloalkoxy, aryloxy, aralkyloxy, alkylthio, carboxamido, sulfonamido, alkylcarbonyl, arylcarbonyl, alkyl sulfonyl, aryl sulfonyl, carboxy, carboxyalkyl, optionally substituted cycloalkyl, optionally substituted aryl, optionally substituted heteroaryl, or optionally substituted heterocyclo.

For the purpose of the present disclosure, the term “alkynyl” as used by itself or as part of another group refers to an alkyl group as defined above containing one to three carbon-to-carbon triple bonds. The alkynyl can have one carbon-to-carbon triple bond. The alkynyl group can be chosen from a C₂₋₆ alkynyl group and a C₂₋₄ alkynyl group. Non-limiting exemplary alkynyl groups include ethynyl, propynyl, butynyl, 2-butynyl, pentynyl, and hexynyl groups.

For the purpose of the present disclosure, the term “optionally substituted alkynyl” as used herein by itself or as part of another group means the alkynyl as defined above is either unsubstituted or substituted with one, two or three substituents independently chosen from halo, nitro, cyano, hydroxy, amino, alkylamino, dialkylamino, haloalkyl, hydroxyalkyl, alkoxy, haloalkoxy, aryloxy, aralkyloxy, alkylthio, carboxamido, sulfonamido, alkylcarbonyl, arylcarbonyl, alkyl sulfonyl, aryl sulfonyl, carboxy, carboxyalkyl, cycloalkyl, aryl, heteroaryl, or heterocyclo.

For the purpose of the present disclosure, the term “haloalkyl” as used by itself or as part of another group refers to an alkyl group substituted by one or more fluorine, chlorine, bromine and/or iodine atoms. The alkyl group can be substituted by one, two, or three fluorine and/or chlorine atoms. The haloalkyl group can be chosen from a C₁₋₄ haloalkyl group. Non-limiting exemplary haloalkyl groups include fluoromethyl, difluoromethyl, trifluoromethyl, pentafluoroethyl, 1,1-difluoroethyl, 2,2-difluoroethyl, 2,2,2-trifluoroethyl, 3,3,3-trifluoropropyl, 4,4,4-trifluorobutyl, and trichloromethyl groups.

For the purpose of the present disclosure, the term “hydroxyalkyl” as used by itself or as part of another group refers to an alkyl group substituted with one or more, e.g., one, two, or three, hydroxy groups. The hydroxyalkyl group can be chosen from a monohydroxyalkyl group, i.e., substituted with one hydroxy group, a dihydroxyalkyl group, i.e., substituted with two hydroxy groups, and a C₁₋₄ hydroxyalkyl group. Non-limiting exemplary hydroxyalkyl groups include hydroxymethyl, hydroxyethyl, hydroxypropyl and hydroxybutyl groups, such as 1-hydroxyethyl, 2-hydroxyethyl, 1,2-dihydroxyethyl, 2-hydroxypropyl, 3-hydroxypropyl, 3-hydroxybutyl, 4-hydroxybutyl, 2-hydroxy-1-methylpropyl, and 1,3-dihydroxyprop-2-yl.

For the purpose of the present disclosure, the term “alkoxy” as used by itself or as part of another group refers to an optionally substituted alkyl, optionally substituted cycloalkyl, optionally substituted heterocyclo, optionally substituted alkenyl or optionally substituted alkynyl attached to a terminal oxygen atom. The alkoxy group can be chosen from a C₁₋₄ alkoxy group and a C₁₋₄ alkyl attached to a terminal oxygen atom, e.g., methoxy, ethoxy, and tert-butoxy.

For the purpose of the present disclosure, the term “alkylthio” as used by itself or as part of another group refers to a sulfur atom substituted by an optionally substituted alkyl group. The alkylthio group can be chosen from a C₁₋₄ alkylthio group. Non-limiting exemplary alkylthio groups include —SCH₃ (i.e., methylthio), and —SCH₂CH₃.

For the purpose of the present disclosure, the term “alkoxyalkyl” as used by itself or as part of another group refers to an alkyl group substituted with an alkoxy group. Non-limiting exemplary alkoxyalkyl groups include methoxymethyl, methoxyethyl, methoxypropyl, methoxybutyl, ethoxymethyl, ethoxyethyl, ethoxypropyl, ethoxybutyl, propoxymethyl, iso-propoxymethyl, propoxyethyl, propoxypropyl, butoxymethyl, tert-butoxymethyl, isobutoxymethyl, sec-butoxymethyl, and pentyloxymethyl.

For the purpose of the present disclosure, the term “halo” as used by itself or as part of another group refers to a halogen atom. Non-limiting exemplary halo groups include fluoro, chloro, bromo, and iodo.

For the purpose of the present disclosure, the term “haloalkoxy” as used by itself or as part of another group refers to a haloalkyl attached to a terminal oxygen atom. Non-limiting exemplary haloalkoxy groups include fluoromethoxy, difluoromethoxy, trifluoromethoxy, and 2,2,2-trifluoroethoxy.

For the purpose of the present disclosure, the term “heteroalkyl” as used by itself or part of another group refers to a stable straight or branched chain hydrocarbon radical containing 1 to 10 carbon atoms and at least two heteroatoms, which can be the same or different, selected from O, N, or S, wherein: 1) the nitrogen atom(s) and sulfur atom(s) can optionally be oxidized; and/or 2) the nitrogen atom(s) can optionally be quaternized. The heteroatoms can be placed at any interior position of the heteroalkyl group or at a position at which the heteroalkyl group is attached to the remainder of the molecule. The heteroalkyl group can contain two oxygen atoms, one oxygen and one nitrogen atom, or two nitrogen atoms. Non-limiting exemplary heteroalkyl groups include —CH₂OCH—₂CH₂OCH₃, —OCH₂CH₂OCH₂CH₂OCH₃, —CH₂NHCH₂CH₂OCH₂, —OCH₂CH₂NH₂, —NHCH₂CH₂N(H)CH₃, —NHCH₂CH₂OCH₃ and —OCH₂CH₂OCH₃.

For the purpose of the present disclosure, the term “aryl” as used by itself or as part of another group refers to a monocyclic or bicyclic aromatic ring system having from six to fourteen carbon atoms (i.e., C₆₋₁₄ aryl). The aryl group can be chosen from a C₆₋₁₄ aryl group and a C₆₋₁₀ aryl group. Non-limiting exemplary aryl groups include phenyl (abbreviated as “Ph”), naphthyl, phenanthryl, anthracyl, indenyl, azulenyl, biphenyl, biphenylenyl, and fluorenyl groups. The aryl group can be chosen from phenyl or naphthyl. The aryl group can be phenyl.

For the purpose of the present disclosure, the term “optionally substituted aryl” as used herein by itself or as part of another group means that the aryl as defined above is either unsubstituted or substituted with one to five substituents independently selected from the group consisting of halo, nitro, cyano, hydroxy, amino, alkylamino, dialkylamino, haloalkyl, hydroxyalkyl, alkoxy, haloalkoxy, aryloxy, heteroaryloxy, aralkyl aralkyloxy, alkylthio, carboxamido, sulfonamido, alkylcarbonyl, arylcarbonyl, alkyl sulfonyl, arylsulfonyl, carboxy, carboxyalkyl, alkyl, optionally substituted cycloalkyl, alkenyl, alkynyl, optionally substituted aryl, optionally substituted heteroaryl, optionally substituted heterocyclo, alkoxyalkyl, (amino)alkyl, hydroxyalkylamino, (alkylamino)alkyl, (dialkylamino)alkyl, (cyano)alkyl, (carboxamido)alkyl, mercaptoalkyl, (heterocyclo)alkyl, (cycloalkylamino)alkyl, (C₁₋₄haloalkoxy)alkyl, (heteroaryl)alkyl. The optionally substituted aryl can be an optionally substituted phenyl. The optionally substituted phenyl can have four substituents, three substituents, two substituents, or one substituent. The optionally substituted phenyl can have one amino, alkylamino, dialkylamino, (amino)alkyl, (alkylamino)alkyl, or (dialkylamino)alkyl substituent. Non-limiting exemplary substituted aryl groups include 2-methylphenyl, 2-methoxyphenyl, 2-fluorophenyl, 2-chlorophenyl, 2-bromophenyl, 3-methylphenyl, 3-methoxyphenyl, 3-fluorophenyl, 3-chlorophenyl, 4-methylphenyl, 4-ethylphenyl, 4-methoxyphenyl, 4-fluorophenyl, 4-chlorophenyl, 2,6-di-fluorophenyl, 2,6-di-chlorophenyl, 2-methyl, 3-methoxyphenyl, 2-ethyl, 3-methoxyphenyl, 3,4-di-methoxyphenyl, 3,5-di-fluorophenyl 3,5-di-methylphenyl, 3,5-dimethoxy, 4-methylphenyl, 2-fluoro-3-chlorophenyl, 3-chloro-4-fluorophenyl, and 2-phenylpropan-2-amine. The term optionally substituted aryl is meant to include groups having fused optionally substituted cycloalkyl and fused optionally substituted heterocyclo rings. Examples include:

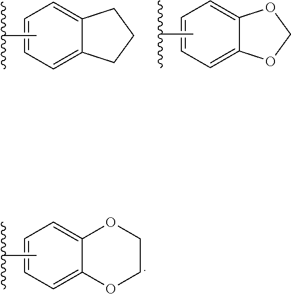

For the purpose of the present disclosure, the term “aryloxy” as used by itself or as part of another group refers to an optionally substituted aryl attached to a terminal oxygen atom. A non-limiting exemplary aryloxy group is PhO—.

For the purpose of the present disclosure, the term “heteroaryloxy” as used by itself or as part of another group refers to an optionally substituted heteroaryl attached to a terminal oxygen atom.

For the purpose of the present disclosure, the term “aralkyloxy” or “arylalkyloxy” as used by itself or as part of another group refers to an aralkyl group attached to a terminal oxygen atom. A non-limiting exemplary aralkyloxy group is PhCH₂O—.

For the purpose of the present disclosure, the term “heteroaryl” or “heteroaromatic” refers to monocyclic and bicyclic aromatic ring systems having 5 to 14 ring atoms (i.e., C₅₋₁₄ heteroaryl) and 1, 2, 3, or 4 heteroatoms independently chosen from oxygen, nitrogen or sulfur. The heteroaryl group can be chosen from a C₅₋₁₄ heteroaryl group and a C₃₋₆ heteroaryl group. The heteroaryl can have three heteroatoms, two heteroatoms, or one heteroatom. The heteroaryl can be a Cs heteroaryl, or a C₆ heteroaryl. Non-limiting exemplary heteroaryl groups include thienyl, benzo[b]thienyl, naphtho[2,3-b]thienyl, thianthrenyl, furyl, benzofuryl, pyranyl, isobenzofuranyl, benzooxazonyl, chromenyl, xanthenyl, 2H-pyrrolyl, pyrrolyl, imidazolyl, pyrazolyl, pyridyl, pyrazinyl, pyrimidinyl, pyridazinyl, triazinyl, isoindolyl, 3H-indolyl, indolyl, indazolyl, purinyl, isoquinolyl, quinolyl, phthalazinyl, naphthyridinyl, cinnolinyl, quinazolinyl, pteridinyl, 4aH-carbazolyl, carbazolyl, β-carbolinyl, phenanthridinyl, acridinyl, phenanthrolinyl, phenazinyl, thiazolyl, isothiazolyl, phenothiazolyl, isoxazolyl, furazanyl, triazolyl, tetrazolyl, and phenoxazinyl. The heteroaryl can be chosen from thienyl (e.g., thien-2-yl and thien-3-yl), furyl (e.g., 2-furyl and 3-furyl), pyrrolyl (e.g., 1H-pyrrol-2-yl and 1H-pyrrol-3-yl), imidazolyl (e.g., 2H-imidazol-2-yl and 2H-imidazol-4-yl), pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrimidinyl (e.g., pyrimidin-2-yl, pyrimidin-4-yl, and pyrimidin-5-yl), triazinyl (e.g., 1,3,5-triazinyl, 1,2,4-triazinyl, and 1,2,3-triazinyl), thiazolyl (e.g., thiazol-2-yl, thiazol-4-yl, and thiazol-5-yl), isothiazolyl (e.g., isothiazol-3-yl, isothiazol-4-yl, and isothiazol-5-yl), oxazolyl (e.g., oxazol-2-yl, oxazol-4-yl, and oxazol-5-yl) isoxazolyl (e.g., isoxazol-3-yl, isoxazol-4-yl, and isoxazol-5-yl), triazolyl (e.g., 1,2,4-triazolyl and 1,2,3-triazolyl). The term “heteroaryl” is also meant to include possible N-oxides. Exemplary N-oxides include pyridyl N-oxide.

For the purpose of the present disclosure, the term “optionally substituted heteroaryl” as used by itself or as part of another group means that the heteroaryl as defined above is either unsubstituted or substituted with one to four substituents, e.g., one or two substituents, independently chosen from halo, nitro, cyano, hydroxy, amino, alkylamino, dialkylamino, haloalkyl, hydroxyalkyl, alkoxy, haloalkoxy, aralkyl aryloxy, aralkyloxy, alkylthio, carboxamido, sulfonamido, alkylcarbonyl, arylcarbonyl, alkyl sulfonyl, arylsulfonyl, carboxy, carboxyalkyl, alkyl, optionally substituted cycloalkyl, alkenyl, alkynyl, optionally substituted aryl, optionally substituted heteroaryl, optionally substituted heterocyclo, alkoxyalkyl, (amino)alkyl, hydroxyalkylamino, (alkylamino)alkyl, (dialkylamino)alkyl, (cyano)alkyl, (carboxamido)alkyl, mercaptoalkyl, (heterocyclo)alkyl, (heteroaryl)alkyl, —N(R³³)(R³⁴), or —N(H)C(═O)—R³⁵, wherein R³³ is hydrogen or C₁₋₄ alkyl; R³⁴ is alkoxyalkyl, (heterocyclo)alkyl, (amino)alkyl, (alkylamino)alkyl, or (dialkylamino)alkyl; and R³⁵ is alkyl, optionally substituted aryl, or optionally substituted heteroaryl. The optionally substituted heteroaryl can have one substituent. The substituent can be amino, alkylamino, dialkylamino, (amino)alkyl, hydroxyalkylamino, (alkylamino)alkyl, (dialkylamino)alkyl, (heterocyclo)alkyl, —N(R³³)(R³⁴), or —N(H)C(═O)—R³⁵. The optionally substituted heteroaryl can be an optionally substituted pyridyl, i.e., 2-, 3-, or 4-pyridyl. Any available carbon or nitrogen atom can be substituted.

For the purpose of the present disclosure, the term “heterocycle” or “heterocyclo” as used by itself or as part of another group refers to saturated and partially unsaturated (e.g., containing one or two double bonds) cyclic groups containing one, two, or three rings having from three to fourteen ring members (i.e., a 3- to 14-membered heterocyclo) and at least one heteroatom. The heterocyclo group can be chosen from a C₃₋₁₄ heterocyclo group and a C₃₋₈ heterocyclo group. Each heteroatom is independently selected from the group consisting of oxygen, sulfur, including sulfoxide and sulfone, and/or nitrogen atoms, which can be quaternized. The term “heterocyclo” is meant to include cyclic ureido groups such as imidazolidinyl-2-one, cyclic amide groups such as β-lactam, γ-lactam, δ-lactam and c-lactam, and cyclic carbamate groups such as oxazolidinyl-2-one. The term “heterocyclo” is also meant to include groups having fused optionally substituted aryl groups, e.g., indolinyl, indolinyl-2-one, benzo[d]oxazolyl-2(3H)-one. The term “heterocyclo” is also meant to include groups having fused optionally substituted heteroaryl groups, e.g., 5,6,7,8-tetrahydroimidazo[1,5-a]pyrazine. The heterocyclo group can be chosen from a 4-, 5-, 6-, 7- or 8-membered cyclic group containing one ring and one or two oxygen and/or nitrogen atoms, a 5- or 6-membered cyclic group containing one ring and one or two nitrogen atoms, an 8-, 9-, 10-, 11-, or 12-membered cyclic group containing two rings and one or two nitrogen atoms. The heterocyclo can be optionally linked to the rest of the molecule through a carbon or nitrogen atom. Non-limiting exemplary heterocyclo groups include 2-oxopyrrolidin-3-yl, 2-imidazolidinone, piperidinyl, morpholinyl, piperazinyl, pyrrolidinyl, azetidinyl, 8-azabicyclo[3.2.1]octane (nortropane), 6-azaspiro[2.5]octane, 6-azaspiro[3.4]octane, indolinyl, indolinyl-2-one, 1,3-dihydro-2H-benzo[d]imidazol-2-one.

For the purpose of the present disclosure, the term “optionally substituted heterocyclo” as used herein by itself or part of another group means the heterocyclo as defined above is either unsubstituted or substituted with one to four substituents independently selected from halo, nitro, cyano, hydroxy, amino, alkylamino, dialkylamino, haloalkyl, hydroxyalkyl, alkoxy, haloalkoxy, aryloxy, aralkyl aralkyloxy, alkylthio, carboxamido, sulfonamido, alkylcarbonyl, arylcarbonyl, alkyl sulfonyl, aryl sulfonyl, carboxy, carboxyalkyl, alkyl, cycloalkyl, alkenyl, alkynyl, aryl, heteroaryl, heterocyclo, alkoxyalkyl, (amino)alkyl, hydroxyalkylamino, (alkylamino)alkyl, (dialkylamino)alkyl, (cyano)alkyl, (carboxamido)alkyl, mercaptoalkyl, (heterocyclo)alkyl, and (heteroaryl)alkyl. Substitution may occur on any available carbon or nitrogen atom, and may form a spirocycle.

For the purpose of the present disclosure, the term “heterocyclooxy” as used by itself or as part of another group refers to a heterocyclo group attached to a terminal oxygen atom.

For the purpose of the present disclosure, the term “amino” as used by itself or as part of another group refers to —NH₂.

For the purpose of the present disclosure, the term “alkylamino” as used by itself or as part of another group refers to —NHR²¹, wherein R²¹ is C₁₋₆ alkyl. R²¹ can be C₁₋₄ alkyl. Non-limiting exemplary alkylamino groups include —N(H)CH₃ and —N(H)CH₂CH₃.

For the purpose of the present disclosure, the term “dialkylamino” as used by itself or as part of another group refers to —NR^{22a}R^{22b}, wherein R^{22a} and R^{22b} are each independently C₁₋₆ alkyl. R^{22a} and R^{22b} can each independently be C₁₋₄ alkyl. Non-limiting exemplary dialkylamino groups include —N(CH₃)₂ and —N(CH₃)CH₂CH(CH₃)₂.

For the purpose of the present disclosure, the term “hydroxyalkylamino” as used by itself or as part of another group refers to —NHR²³, wherein R²³ is hydroxyalkyl.

For the purpose of the present disclosure, the term “cycloalkylamino” as used by itself or as part of another group refers to —NR^{24a}R^{24b}, wherein R^{24a} is optionally substituted cycloalkyl and R^{24b} is hydrogen or C₁₋₄ alkyl.

For the purpose of the present disclosure, the term “aralkylamino” as used by itself or as part of another group refers to —NR^{25a}R^{25b}, wherein R^{25a} is aralkyl and R^{25b} is hydrogen or C₁₋₄ alkyl. Non-limiting exemplary aralkylamino groups include —N(H)CH₂Ph and —N(CH₃)CH₂Ph.

For the purpose of the present disclosure, the term “(amino)alkyl” as used by itself or as part of another group refers to an alkyl group substituted with an amino group. The alkyl can be a C₁₋₄ alkyl. Non-limiting exemplary (amino)alkyl groups include —CH₂NH₂, —C(NH₂)(H)CH₃, —CH₂CH₂NH₂, —CH₂C(NH₂)(H)CH₃, —CH₂CH₂CH₂NH₂, —CH₂CH₂CH₂CH₂NH₂, and —CH₂C(CH₃)₂CH₂NH₂

For the purpose of the present disclosure, the term “(alkylamino)alkyl” as used by itself or as part of another group refers to an alkyl group substituted with an alkylamino group. The alkyl can be a C₁₋₄ alkyl. A non-limiting exemplary (alkylamino)alkyl group is —CH₂CH₂N(H)CH₃.

For the purpose of the present disclosure, the term “(dialkylamino)alkyl” as used by itself or as part of another group refers to an alkyl group substituted by a dialkylamino group. The alkyl can be a C₁₋₄ alkyl. Non-limiting exemplary (dialkylamino)alkyl groups are —CH₂CH₂N(CH₃)₂.

For the purpose of the present disclosure, the term “(cycloalkylamino)alkyl” as used by itself or as part of another group refers to an alkyl group substituted by a cycloalkylamino group. The alkyl can be a C₁₋₄ alkyl. Non-limiting exemplary (cycloalkylamino)alkyl groups include —CH₂N(H)cyclopropyl, —CH₂N(H)cyclobutyl, and —CH₂N(H)cyclohexyl.

For the purpose of the present disclosure, the term “(aralkylamino)alkyl” as used by itself or as part of another group refers to an alkyl group substituted with an aralkylamino group. The alkyl can be a C₁₋₄ alkyl. A non-limiting exemplary (aralkylamino)alkyl group is —CH₂CH₂CH₂N(H)CH₂Ph.

For the purpose of the present disclosure, the term “(cyano)alkyl” as used by itself or as part of another group refers to an alkyl group substituted with one or more cyano, e.g., —CN, groups. The alkyl can be a C₁₋₄ alkyl. Non-limiting exemplary (cyano)alkyl groups include —CH₂CH₂CN, —CH₂CH₂CH₂CN, and —CH₂CH₂CH₂CH₂CN.

For the purpose of the present disclosure, the term “(amino)(hydroxy)alkyl” as used by itself or as part of another group refers to an alkyl group substituted with one amino, alkylamino, or dialkylamino group and one hydroxy group. The alkyl is a C₁₋₆ alkyl or a C₁₋₄ alkyl.

For the purpose of the present disclosure, the term “(amino)(aryl)alkyl” as used by itself or as part of another group refers to an alkyl group substituted with one amino, alkylamino, or dialkylamino group and one optionally substituted aryl group. The alkyl can be a C₁₋₆ alkyl. The optionally substituted aryl group can be an optionally substituted phenyl.

For the purpose of the present disclosure, the term “(cycloalkyl)alkyl” as used by itself or as part of another group refers to an alkyl group substituted with one optionally substituted cycloalkyl group. The alkyl can be a C₁₋₄ alkyl or a C₃₋₆ cycloalkyl. The optionally substituted cycloalkyl group can be substituted with an amino or (amino)alkyl group.

For the purpose of the present disclosure, the term “(cycloalkyl)alkyloxy” as used by itself or as part of another group refers to a (cycloalkyl)alkyl group attached to a terminal oxygen atom. The alkyloxy can be a C₁₋₄ alkyloxy or a C₃₋₆ cycloalkyloxy.

For the purpose of the present disclosure, the term “(hydroxy)(aryl)alkyl” as used by itself or as part of another group refers to an alkyl group substituted with one hydroxy group and one optionally substituted aryl group. The alkyl can be a C₁₋₆ alkyl. The optionally substituted aryl group can be an optionally substituted phenyl. Non-limiting exemplary (hydroxy)(aryl)alkyl groups include:

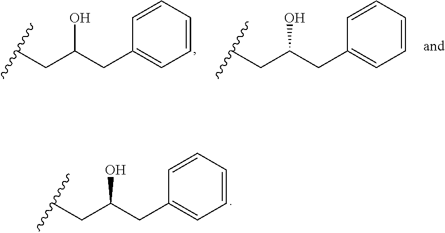

For the purpose of the present disclosure, the term “carboxamido” as used by itself or as part of another group refers to a radical of formula —C(═O)NR^{26a}R^{26b}, wherein R^{26a} and R^{26b} are each independently hydrogen, optionally substituted alkyl, optionally substituted aryl, or optionally substituted heteroaryl, or R^{26a} and R^{26b} taken together with the nitrogen to which they are attached from a 3- to 8-membered heterocyclo group. R^{26a} and R^{26b} can each independently be hydrogen or optionally substituted alkyl. Non-limiting exemplary carboxamido groups include —CONH₂, —CON(H)CH₃, —CON(CH₃)₂, and —CON(H)Ph.

For the purpose of the present disclosure, the term “(carboxamido)alkyl” as used by itself or as part of another group refers to an alkyl group substituted with a carboxamido group. Non-limiting exemplary (carboxamido)alkyl groups include —CH₂CONH₂, —C(H)CH₃—CONH₂, and —CH₂CON(H)CH₃.

For the purpose of the present disclosure, the term “sulfonamido” as used by itself or as part of another group refers to a radical of the formula —SO₂NR^{27a}R^{27b}, wherein R^{27a} and R^{27b} are each independently hydrogen, optionally substituted alkyl, or optionally substituted aryl, or R^{27a} and R^{27b} taken together with the nitrogen to which they are attached from a 3- to 8-membered heterocyclo group. Non-limiting exemplary sulfonamido groups include —SO₂NH₂, —SO₂N(H)CH₃, and —SO₂N(H)Ph.

For the purpose of the present disclosure, the term “alkylcarbonyl” as used by itself or as part of another group refers to a carbonyl group, i.e., —C(═O)—, substituted by an alkyl group. A non-limiting exemplary alkylcarbonyl group is —COCH₃.

For the purpose of the present disclosure, the term “arylcarbonyl” as used by itself or as part of another group refers to a carbonyl group, i.e., —C(═O)—, substituted by an optionally substituted aryl group. A non-limiting exemplary arylcarbonyl group is -COPh.

For the purpose of the present disclosure, the term “alkylsulfonyl” as used by itself or as part of another group refers to a sulfonyl group, i.e., —SO₂—, substituted by any of the above-mentioned optionally substituted alkyl groups. Non-limiting exemplary alkylsulfonyl groups are —SO2CH₃ (i.e., methylsulfonyl) and —SO₂CH₂CH₃ (i.e., ethyl sulfonyl).

For the purpose of the present disclosure, the term “arylsulfonyl” as used by itself or as part of another group refers to a sulfonyl group, i.e., —SO₂—, substituted by any of the above-mentioned optionally substituted aryl groups. A non-limiting exemplary arylsulfonyl group is —SO₂Ph.

For the purpose of the present disclosure, the term “mercaptoalkyl” as used by itself or as part of another group refers to any of the above-mentioned alkyl groups substituted by a —SH group.

For the purpose of the present disclosure, the term “carboxy” as used by itself or as part of another group refers to a radical of the formula —COOH.

For the purpose of the present disclosure, the term “carboxyalkyl” as used by itself or as part of another group refers to any of the above-mentioned alkyl groups substituted with a —COOH. A non-limiting exemplary carboxyalkyl group is —CH₂CO₂H.

For the purpose of the present disclosure, the term “alkoxycarbonyl” as used by itself or as part of another group refers to a carbonyl group, i.e., —C(═O)—, substituted by an alkoxy group. Non-limiting exemplary alkoxycarbonyl groups are —CO₂Me and —CO₂Et.

For the purpose of the present disclosure, the term “aralkyl” or “arylalkyl” as used by itself or as part of another group refers to an alkyl group substituted with one, two, or three optionally substituted aryl groups. The aralkyl group can be a C₁₋₄ alkyl substituted with one optionally substituted aryl group. Non-limiting exemplary aralkyl groups include benzyl, phenethyl, —CHPh₂, —CH₂(4-OH—Ph), and —CH(4-F—Ph)₂.

For the purpose of the present disclosure, the term “(heterocyclo)alkyl” as used by itself or as part of another group refers to an alkyl group substituted with one, two, or three optionally substituted heterocyclo groups. The (heterocyclo)alkyl can be a C₁₋₄ alkyl substituted with one optionally substituted heterocyclo group. The heterocyclo can be linked to the alkyl group through a carbon or nitrogen atom. Non-limiting exemplary (heterocyclo)alkyl groups include:

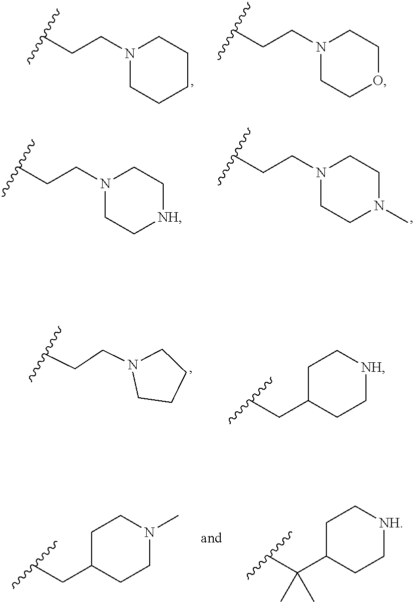

For the purpose of the present disclosure, the term “heterocycloalkyloxy” or “(heterocyclo)alkyloxy” as used by itself or as part of another group refers to a heterocycloalkyl group attached to a terminal oxygen atom. A non-limiting exemplary heterocycloalkyloxy is:

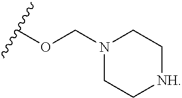 [SMILES: *OCN1CCNCC1]

For the purpose of the present disclosure, the term “heteroaralkyl” or “(heteroaryl)alkyl” as used by itself or as part of another group refers to an alkyl group substituted with one, two, or three optionally substituted heteroaryl groups. The (heteroaryl)alkyl group can be a C₁₋₄ alkyl substituted with one optionally substituted heteroaryl group. Non-limiting exemplary (heteroaryl)alkyl groups include:

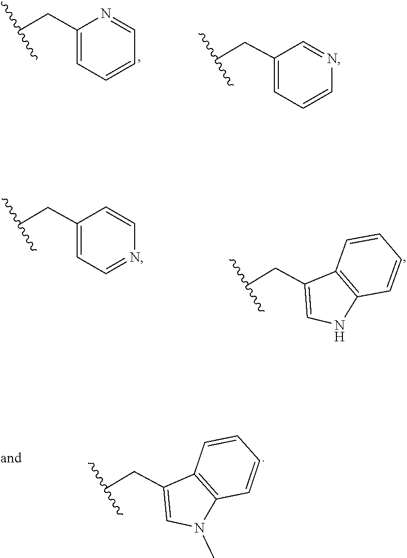 [SMILES: *Cc1ccccn1 ; *Cc1cccnc1 ; *Cc1ccncc1 ; *Cc1c[nH]c2ccccc12 ; *Cc1cn(C)c2ccccc12]

For the purpose of the present disclosure, the term “heteroaralkyloxy” or “heteroarylalkyloxy” as used by itself or as part of another group refers to a heteroaralkyl group attached to a terminal oxygen atom. A non-limiting exemplary of a heteroaralkyloxy group is:

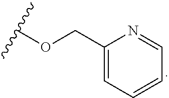 [SMILES: *OCc1ccccn1]

For the purpose of the present disclosure, the term “alkylcarbonylamino” as used by itself or as part of another group refers to an alkylcarbonyl group attached to an amino. A non-limiting exemplary alkylcarbonylamino group is —NHCOCH₃.

The present disclosure encompasses any of the Compounds of the Disclosure being isotopically-labelled (i.e., radiolabeled) by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as ²H (or deuterium (D)), ³H, ¹¹C, ¹³C, ¹⁴C, ¹⁵N, ¹⁸O, ¹⁷O, ³¹P, ³²P, ³⁵S, ¹⁸F, and ³⁶Cl, respectively, e.g., ³H, ¹¹C, and ¹⁴C. The present disclosure also provides a composition wherein substantially all of the atoms at a position within the Compound of the Disclosure are replaced by an atom having a different atomic mass or mass number. The present disclosure also provides a composition wherein a portion of the atoms at a position within the Compound of the disclosure are replaced, i.e., the Compound of the Disclosure is enriched at a position with an atom having a different atomic mass or mass number. In one embodiment, the present disclosure provides a composition wherein a Compound of the Disclosure has from 1 to 8 hydrogens replaced with deuterium. Isotopically-labelled Compounds of the Disclosure can be prepared by methods known in the art.

Compounds of the Disclosure may contain one or more asymmetric centers and may thus give rise to enantiomers, diastereomers, and other stereoisomeric forms. The present disclosure is meant to encompass the use of all such possible forms, as well as their racemic and resolved forms and mixtures thereof. The individual enantiomers can be separated according to methods known in the art in view of the present disclosure. When the compounds described herein contain olefinic double bonds or other centers of geometric asymmetry, and unless specified otherwise, it is intended that they include both E and Z geometric isomers. All tautomers are intended to be encompassed by the present disclosure as well.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” or “asymmetric carbon atom” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” refers to a mixture of equal parts of enantiomers and which mixture is optically inactive.

The term “absolute configuration” refers to the spatial arrangement of the atoms of a chiral molecular entity (or group) and its stereochemical description, e.g., R or S.

The stereochemical terms and conventions used in the specification are meant to be consistent with those described in Pure & Appl. Chem 68:2193 (1996), unless otherwise indicated.

The term “enantiomeric excess” or “ee” refers to a measure for how much of one enantiomer is present compared to the other. For a mixture of R and S enantiomers, the percent enantiomeric excess is defined as |R−S|*100, where R and S are the respective mole or weight fractions of enantiomers in a mixture such that R+S=1. With knowledge of the optical rotation of a chiral substance, the percent enantiomeric excess is defined as ([α]_{obs}/[α]ₘₐₓ)*100, where [α]_{obs} is the optical rotation of the mixture of enantiomers and [α]ₘₐₓ is the optical rotation of the pure enantiomer. Determination of enantiomeric excess is possible using a variety of analytical techniques, including NMR spectroscopy, chiral column chromatography or optical polarimetry.

The terms “enantiomerically pure” or “enantiopure” refer to a sample of a chiral substance all of whose molecules (within the limits of detection) have the same chirality sense.

The terms “enantiomerically enriched” or “enantioenriched” refer to a sample of a chiral substance whose enantiomeric ratio is greater than 50:50. Enantiomerically enriched compounds may be enantiomerically pure.

It is understood that embodiments of the invention described herein include “consisting” and/or “consisting essentially of” embodiments. As used herein, the singular form “a,” “an,” and “the” includes plural references unless indicated otherwise. Use of the term “or” herein is not meant to imply that alternatives are mutually exclusive.

In this application, the use of “or” means “and/or” unless expressly stated or understood by one skilled in the art. In the context of a multiple dependent claim, the use of “or” refers back to more than one preceding independent or dependent claim.

The term “about,” as used herein, includes the recited number ±10%. Thus, “about 10” means 9 to 11. As is understood by one skilled in the art, reference to “about” a value or parameter herein includes (and describes) instances that are directed to that value or parameter per se. For example, description referring to “about X” includes description of “X.”

The present disclosure encompasses the preparation and use of salts of the Compounds of the Disclosure, including non-toxic pharmaceutically acceptable salts. Examples of pharmaceutically acceptable addition salts include inorganic and organic acid addition salts and basic salts. The pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, phosphate, sulphate and the like; organic acid salts such as citrate, lactate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparginate, glutamate and the like. The term “pharmaceutically acceptable salt” as used herein, refers to any salt, e.g., obtained by reaction with an acid or a base, of a Compound of the Disclosure that is physiologically tolerated in the target patient (e.g., a mammal, e.g., a human).

Acid addition salts can be formed by mixing a solution of the particular Compound of the Disclosure with a solution of a pharmaceutically acceptable non-toxic acid such as hydrochloric acid, fumaric acid, maleic acid, succinic acid, acetic acid, citric acid, tartaric acid, carbonic acid, phosphoric acid, oxalic acid, dichloroacetic acid, or the like. Basic salts can be formed by mixing a solution of the compound of the present disclosure with a solution of a pharmaceutically acceptable non-toxic base such as sodium hydroxide, potassium hydroxide, choline hydroxide, sodium carbonate and the like.

The present disclosure encompasses the preparation and use of solvates of Compounds of the Disclosure. Solvates typically do not significantly alter the physiological activity or toxicity of the compounds, and as such may function as pharmacological equivalents. The term “solvate” as used herein is a combination, physical association and/or solvation of a compound of the present disclosure with a solvent molecule such as, e.g. a disolvate, monosolvate or hemisolvate, where the ratio of solvent molecule to compound of the present disclosure is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. Compounds of the Disclosure can be present as solvated forms with a pharmaceutically acceptable solvent, such as water, methanol, ethanol, and the like, and it is intended that the disclosure includes both solvated and unsolvated forms of Compounds of the Disclosure. One type of solvate is a hydrate. A “hydrate” relates to a particular subgroup of solvates where the solvent molecule is water. Solvates typically can function as pharmacological equivalents. Preparation of solvates is known in the art. See, for example, M. Caira et al, J. Pharmaceut. Sci., 93(3):601-611 (2004), which describes the preparation of solvates of fluconazole with ethyl acetate and with water. Similar preparation of solvates, hemisolvates, hydrates, and the like are described by E.C. van Tonder et al., AAPS Pharm. Sci. Tech., 5(1):Article 12 (2004), and A. L. Bingham et al., Chem. Commun. 603-604 (2001). A typical, non-limiting, process of preparing a solvate would involve dissolving a Compound of the Disclosure in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvent in a crystal of the solvate.

Since Compounds of the Disclosure are inhibitors of USP1 proteins, the present disclosure provides a method for inhibiting a USP1 protein comprising contacting a USP1 protein or a composition comprising a USP1 protein with one or more Compounds of the Disclosure.

Since Compounds of the Disclosure are inhibitors of USP1 proteins, a number of diseases, conditions, or disorders mediated by USP1 proteins can be treated by employing these compounds. The present disclosure is thus directed generally to a method for treating a disease, condition, or disorder responsive to the inhibition of USP1 proteins in an animal suffering from, or at risk of suffering from, the disorder, the method comprising administering to the animal an effective amount of one or more Compounds of the Disclosure.

The present disclosure is further directed to a method of inhibiting USP1 proteins in an animal in need thereof, the method comprising administering to the animal a therapeutically effective amount of at least one Compound of the Disclosure.

As used herein, “treatment” is an approach for obtaining beneficial or desired clinical results. “Treatment” as used herein, covers any administration or application of a therapeutic for disease in a mammal, including a human. For purposes of this disclosure, beneficial or desired clinical results include, but are not limited to, any one or more of: alleviation of one or more symptoms, diminishment of extent of disease, preventing or delaying spread (for example, metastasis) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

In the context of cancer, the term “treating” includes, but is not limited to, inhibiting growth of cancer cells, inhibiting replication of cancer cells, lessening of overall tumor burden, and delaying, halting, or slowing tumor growth, progression, or metastasis.

As used herein, “delaying” means to defer, hinder, slow, retard, stabilize, suppress and/or postpone development or progression of the disease (such as cancer). This delay can be of varying lengths of time, depending on the history of the disease and/or individual being treated.

A “therapeutically effective amount” of a substance can vary according to factors such as the disease state, age, sex, and weight of the individual, and the ability of the substance to elicit a desired response in the individual. A therapeutically effective amount is also one in which any toxic or detrimental effects of the substance are outweighed by the therapeutically beneficial effects. A therapeutically effective amount can be delivered in one or more administrations. A therapeutically effective amount refers to an amount effective, at dosages and for periods of time necessary, to achieve the desired therapeutic effect.

The terms “administer,” “administering,” “administration,” and the like refer to methods that can be used to enable delivery of the therapeutic agent to the desired site of biological action. Administration techniques that can be employed with the agents and methods described herein are found in e.g., Goodman and Gilman, The Pharmacological Basis of Therapeutics, current ed.; Pergamon; and Remington's, Pharmaceutical Sciences (current edition), Mack Publishing Co., Easton, Pa.

The terms “pharmaceutical formulation” and “pharmaceutical composition” refer to a preparation which is in such form as to permit the biological activity of the active ingredient(s) to be effective, and which contains no additional components which are unacceptably toxic to a subject to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed.

A “sterile” formulation is aseptic or essentially free from living microorganisms and their spores.

The term “container” means any receptacle and closure therefore suitable for storing, shipping, dispensing, and/or handling a pharmaceutical product.

The term “insert” or “package insert” means information accompanying a pharmaceutical product that provides a description of how to administer the product, along with the safety and efficacy data required to allow the physician, pharmacist, and patient to make an informed decision regarding use of the product. The package insert generally is regarded as the “label” for a pharmaceutical product.

The term “disease” or “condition” or “disorder” as used herein refers to a condition where treatment is needed and/or desired and denotes disturbances and/or anomalies that as a rule are regarded as being pathological conditions or functions, and that can manifest themselves in the form of particular signs, symptoms, and/or malfunctions. As demonstrated below, Compounds of the Disclosure inhibit USP1 proteins and can be used in treating diseases and conditions such as proliferative diseases, wherein inhibition of USP1 proteins provides a benefit.

The terms “polypeptide” and “protein” are used interchangeably to refer to a polymer of amino acid residues and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

“USP1” and “ubiquitin-specific-processing protease 1” as used herein refer to any native polypeptide or USP1-encoding polynucleotide. The term “USP1” encompasses “full-length,” unprocessed USP1 polypeptide as well as any forms of USP1 that result from processing within the cell (e.g., removal of the signal peptide). The term also encompasses naturally occurring variants of USP1, e.g., those encoded by splice variants and allelic variants. The USP1 polypeptides described herein can be isolated from a variety of sources, such as from human tissue types or from another source, or prepared by recombinant or synthetic methods. Human USP1 sequences are known and include, for example, the sequences publicly available as UniProt No. O94782 (including isoforms). As used herein, the term “human USP1 protein” refers to USP1 protein comprising the amino acid sequence as set forth in SEQ ID NO:1 in U.S. provisional patent application No. 62/857,986 filed Jun. 6, 2019.

USP1 is a deubiquitinating enzyme that acts as part of a complex with UAF1. USP1's “deubiquitinase activity” includes its ability to deubiquitinate as part of the USP1-UAF1 complex.

The term “specifically binds” to a protein or domain of a protein is a term that is well understood in the art, and methods to determine such specific binding are also well known in the art. A molecule is said to exhibit “specific binding” or “preferential binding” if it reacts or associates more frequently, more rapidly, with greater duration and/or with greater affinity with a particular protein or domain of a protein than it does with alternative proteins or domains. It should be understood that a molecule that specifically or preferentially binds to a first protein or domain may or may not specifically or preferentially bind to a second protein or domain. As such, “specific binding” or “preferential binding” does not necessarily require (although it can include) exclusive binding. Generally, but not necessarily, reference to binding means preferential binding. For example, a USP1 inhibitor that specifically binds to USP1, UAF1, and/or the USP1-UAF1 complex may not bind to other deubiquitinases, other USP proteins, or other UAF1 complexes (e.g., USP46-UAF1) or may bind to other deubiquitinases, other USP proteins, or other UAF1 complexes (e.g., USP46-UAF1) with a reduced affinity as compared to binding to USP1.

The terms “reduction” or “reduce” or “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. In some embodiments, by “reduce” or “inhibit” is meant the ability to cause an overall decrease of 20% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control over the same period of time.

In some embodiments inhibiting USP1 proteins is the inhibition of one or more activities or functions of USP1 proteins. It should be appreciated that the activity or function of the one or more USP1 proteins may be inhibited in vitro or in vivo. Non-limiting examples of activities and functions of USP1 include deubiquitinase activity, and formation of a complex with UAF1 and are described herein. Examplary levels of inhibition of the activity of one or more USP1 proteins include at least 10% inhibiton, at least 20% inhibition, at least 30% inhibition, at least 40% inhibition, at least 50% inhibition, at least 60% inhibition, at least 70% inhibition, at least 80% inhibition, at least 90% inhibition, and up to 100% inhibition.

The terms “individual” or “subject” are used interchangeably herein to refer to an animal; for example, a mammal, such as a human. In some instances, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, an “individual” or “subject” refers to an individual or subject in need of treatment for a disease or disorder. In some instances, the subject to receive the treatment can be a patient, designating the fact that the subject has been identified as having a disorder of relevance to the treatment, or being at particular risk of contracting the disorder.

As used herein, the terms “cancer” and “tumor” refer to or describe the physiological condition in mammals in which a population of cells are characterized by unregulated cell growth. The terms encompass solid and hematological/lymphatic cancers. Examples of cancer include but are not limited to, DNA damage repair pathway deficient cancers. Additional examples of cancer include, but are not limited to, ovarian cancer, breast cancer (including triple negative breast cancer), non-small cell lung cancer (NSCLC), and osteosarcoma. The cancer can be BRCA1 or BRCA2 wildtype. The cancer can also be BRCA1 or BRCA2 mutant. The cancer can further be a PARP inhibitor resistant or refractory cancer, or a PARP inhibitor resistant or refractory BRCA1 or BRCA2-mutant cancer.

As used herein, the term “loss of function” mutation refers to a mutation that that results in the absence of a gene, decreased expression of a gene, or the production of a gene product (e.g. protein) having decreased activity or no activity. Loss of function mutations include for example, missense mutations, nucleotide insertions, nucleotide deletions, and gene deletions. Loss of function mutations also include dominant negative mutations. Thus, cancer cells with a loss of function mutation in a gene encoding p53 include cancer cells that contain missense mutations in a gene encoding p53 as well as cancer cells that lack a gene encoding p53.

### USP1 Inhibitors

In various embodiments, the Compounds of the Disclosure are USP1 inhibitors that reduce the level of USP1 protein and/or inhibit or reduce at least one biological activity of USP1 protein.

In some embodiments, the Compounds of the Disclosure specifically bind to USP1 protein. In some embodiments, the Compounds of the Disclosure specifically bind to USP1 protein in a USP1-UAF1 complex. In some embodiments, the Compounds of the Disclosure specifically bind to USP1 mRNA. In some embodiments, the Compounds of the Disclosure specifically bind to USP1 protein (alone or in a USP1-UAF1 complex) or USP1 mRNA. In some embodiments, the Compounds of the Disclosure specifically bind to UAF1 (alone or in a USP1-UAF1 complex) and inhibit or reduces formation or activity of the USP1-UAF1 complex.

In some embodiments, the Compounds of the Disclosure decrease the formation of the USP1-UAF1 complex. In some embodiments, the Compounds of the Disclosure decrease the activity of the USP1-UAF1 complex. In some embodiments, the Compounds of the Disclosure decrease the deubiquitinase activity of USP1. In some embodiments, the Compounds of the Disclosure increase mono-ubiquitinated PCNA. In some embodiments, the Compounds of the Disclosure increase mono-ubiquitinated FANCD2. In some embodiments, the Compounds of the Disclosure increase mono-ubiquitinated FANCI.

In some embodiments, the Compounds of the Disclosure do not bind to other deubiquitinases, other USP proteins, or other UAF1 complexes (e.g., USP46-UAF1) or bind deubiquitinases, other USP proteins, or other UAF1 complexes (e.g., USP46-UAF1) with at least 5-fold, at least 10-fold, at least 20-fold, or at least 100-fold reduced affinity compared to the affinity for USP1 (i.e., the Ku of the USP1 inhibitor for other deubiquitinases, other USP proteins, or other UAF1 complexes (e.g., USP46-UAF1) is at least 5-fold, at least 10-fold, at least 20-fold, or at least 100-fold higher than the K_{D} for USP1).

In some embodiments, the Compounds of the Disclosure inhibit USP1 deubiquitinase activity with an IC50 of less than about 50 nM, between about 50 nM and about 200 nM, between about 200 nM and about 2 pM, or greater than 2 pM, e.g., as measured using the assay disclosed in US Patent Application Publication No. 2017/0145012 or IC50 of 50 nM to 1000 nM, e.g., as measured using the assay disclosed in Liang et al., Nat Chem Biol 10: 289-304 (2014). In some embodiments, the Compounds of the Disclosure inhibit USP1 deubiquitinase activity with an IC₅₀ as measured using the assay disclosed in Chen, et al., Chem Biol., 18(11):1390-1400 (2011). In some embodiments, the Compounds of the Disclosure do not inhibit the activity of other deubiquitinases, other USP proteins, or other UAF1 complexes (e.g., USP46-UAF1) or inhibit the activity of other deubiquitinases, other USP proteins, or other UAF1 complexes (e.g., USP46-UAF1) with at least 5-fold, at least 10-fold, at least 20-fold, or at least 100-fold higher IC50 compared to the IC50 for inhibition of USP1 deubiquitinase activity.

In some embodiments, the Compounds of the Disclosure bind to a USP1 protein with an affinity in the range of 1 pM to 100 μM, or 1 pM to 1 μM, or 1 pM to 500 nM, or 1 pM to 100 nM. In some embodiment, the Compounds of the Disclosure bind to a USP1 protein with an affinity of about 1 pM to about 100 μM, about 1 nM to about 100 μM, about 1 μM to about 100 μM, about 1 μM to about 50 μM, about 1 μM to about 40 μM, about 1 μM to about 30 μM, about 1 μM to about 20 μM, or about 1 μM to about 10 μM, about 1 μM, about 5 μM, about 10 μM, about 15 μM, about 20 μM, about 25 μM, about 30 μM, about 35 μM, about 40 μM, about 45 μM, about 50 μM, about 60 μM, about 70 μM, about 80 μM, about 90 μM, or about 100 μM. In some embodiment, the Compounds of the Disclosure bind to a USP1 protein with an affinity of about 100 nM to about 1 μM, about 100 nM to about 900 nM, about 100 nM to about 800 nM, about 100 nM to about 700 nM, about 100 nM to about 600 nM, about 100 nM to about 500 nM, about 100 nM to about 400 nM, about 100 nM to about 300 nM, about 100 nM to about 200 nM, about 200 nM to about 1 μM, about 300 nM to about 1 μM, about 400 nM to about 1 μM, about 500 nM to about 1 μM, about 600 nM to about 1 μM, about 700 nM to about 1 μM, about 800 nM to about 1 μM, about 900 nM to about 1 μM, about 100 nM, about 200 nM, about 300 nM, about 400 nM, about 500 nM, about 600 nM, about 700 nM, about 800 nM, or about 900 nM. In some embodiment, the Compounds of the Disclosure bind to a USP1 protein with an affinity of about 1 nM to about 100 nM, 1 nM to about 90 nM, 1 nM to about 80 nM, 1 nM to about 70 nM, 1 nM to about 60 nM, 1 nM to about 50 nM, 1 nM to about 40 nM, 1 nM to about 30 nM, 1 nM to about 20 nM, 1 nM to about 10 nM, about 10 nM to about 100 nM, about 20 nM to about 100 nM, about 30 nM to about 100 nM, about 40 nM to about 100 nM, about 50 nM to about 100 nM, about 60 nM to about 100 nM, about 70 nM to about 100 nM, about 80 nM to about 100 nM, about 90 nM to about 100 nM, about 1 nM, about 2 nM, about 3 nM, about 4 nM, about 5 nM, about 6 nM, about 7 nM, about 8 nM, about 9 nM, about 10 nM, about 20 nM, about 30 nM, about 40 nM, about 50 nM, about 60 nM, about 70 nM, about 80 nM, about 90 nM, or about 100 nM. In some embodiment, the Compounds of the Disclosure bind to a USP1 protein with an affinity of less than 1 μM, less than 500 nM, less than 100 nM, less than 10 nM, or less than 1 nM. In some embodiments, the Compounds of the Disclosure bind to a USP1 protein with an affinity of less than 1 nM.

In some embodiments, the Compounds of the Disclosure inhibit USP1 activity with an IC₅₀ of 1 pM to 100 μM, or 1 pM to 1 μM, or 1 pM to 500 nM, or 1 pM to 100 nM. In some embodiments, the Compounds of the Disclosure inhibit USP1 activity with an IC₅₀ of about 1 pM to about 100 μM, about 1 nM to about 100 μM, about 1 μM to about 100 μM, about 1 μM to about 50 μM, about 1 μM to about 40 μM, about 1 μM to about 30 μM, about 1 μM to about 20 μM, or about 1 μM to about 10 μM, about 1 μM, about 5 μM, about 10 μM, about 15 μM, about 20 μM, about 25 μM, about 30 μM, about 35 μM, about 40 μM, about 45 μM, about 50 μM, about 60 μM, about 70 μM, about 80 μM, about 90 μM, or about 100 μM. In some embodiment, the Compounds of the Disclosure inhibit USP1 activity with an IC₅₀ of about 100 nM to about 1 μM, about 100 nM to about 900 nM, about 100 nM to about 800 nM, about 100 nM to about 700 nM, about 100 nM to about 600 nM, about 100 nM to about 500 nM, about 100 nM to about 400 nM, about 100 nM to about 300 nM, about 100 nM to about 200 nM, about 200 nM to about 1 μM, about 300 nM to about 1 μM, about 400 nM to about 1 μM, about 500 nM to about 1 μM, about 600 nM to about 1 μM, about 700 nM to about 1 μM, about 800 nM to about 1 μM, about 900 nM to about 1 μM, about 100 nM, about 200 nM, about 300 nM, about 400 nM, about 500 nM, about 600 nM, about 700 nM, about 800 nM, or about 900 nM. In some embodiment, the Compounds of the Disclosure inhibit USP1 activity with an IC₅₀ of about 1 nM to about 100 nM, 1 nM to about 90 nM, 1 nM to about 80 nM, 1 nM to about 70 nM, 1 nM to about 60 nM, 1 nM to about 50 nM, 1 nM to about 40 nM, 1 nM to about 30 nM, 1 nM to about 20 nM, 1 nM to about 10 nM, about 10 nM to about 100 nM, about 20 nM to about 100 nM, about 30 nM to about 100 nM, about 40 nM to about 100 nM, about 50 nM to about 100 nM, about 60 nM to about 100 nM, about 70 nM to about 100 nM, about 80 nM to about 100 nM, about 90 nM to about 100 nM, about 1 nM, about 2 nM, about 3 nM, about 4 nM, about 5 nM, about 6 nM, about 7 nM, about 8 nM, about 9 nM, about 10 nM, about 20 nM, about 30 nM, about 40 nM, about 50 nM, about 60 nM, about 70 nM, about 80 nM, about 90 nM, or about 100 nM. In some embodiments, the Compounds of the Disclosure inhibit USP1 activity with an IC₅₀ of less than 1 μM, less than 500 nM, less than 100 nM, less than 10 nM, or less than 1 nM. In some embodiments, the Compounds of the Disclosure inhibit USP1 activity with an IC₅₀ of less than 1 nM.

### Exemplary Assays

Any suitable assay in the art can be used to determine an activity, detect an outcome or effect, determine efficacy, etc. Certain non-limiting exemplary assays that can be used in the methods provided herein are described.

In some instances, a method of determining whether a Compound of the Disclosure inhibits USP1 deubiquitinase activity measure a change in mass upon di-ubiquitin cleavage of deubiquitinase binding. For example, ubiquitin aldehyde and ubiquitin vinyl sulfone form covalent irreversible linkages to deubiquitinases that result in observable mass changes to the deubiquitinases. Similarly, cleavage of di-ubiquitins results in an observable mass change.

In some instances, a method of determining whether a Compound of the Disclosure inhibits USP1 deubiquitinase activity involves an increase in luminescence or fluorescence upon cleavage, e.g., that can be monitored on a plate reader. Such assays can use ubiquitin linked to a flurophore through a linker linkage, such as ubiquitin-7-amino-4-methylcoumarin (Ub-AMC) or ubiquitin-Rhodamine110. Such assays can also use a di-ubiquitin containing an isopeptide linkage. Exemplary di-ubiquitins can comprise a flurophore on one ubiquitin and a quencher on the other ubiquitin such that fluorescence increases with then di-ubiquitin is cleaved. Such assays can also use enzyme coupled systems wherein ubiquitin is coupled to an enzyme that is only active in producing a fluorescence enzyme product when released from the ubiquitin.

Exemplary Deubiquitination Assay for USP1/UAF1 Activity and Inhibitor Testing.

Deubiquitinase activity can be measured using ubiquitin-rhodamine 110 as a substrate. Cleavage of the amide bond between rhodamine and the c-terminal glycine of ubiquitin yields an increase in fluorescence signal. The assay can be conducted in 20 ul total volume of assay buffer (50 mM Tris-HCl, pH 7.8, 0.5 mM EDTA, 0.01% Bovine Serum Albumin, 1 mM DTT, 0.01% Tween-20), and 0.05 nM USP1/UAF1 enzyme. Reaction can be initiated by addition of 150 nM Ubiquitin-rhodamine (Boston Biochem) substrate.

Compounds of the Disclosure can be dissolved in DMSO and tested in dose response format, beginning at 10 μM.

Compounds of the Disclosure can be added to enzyme/assay buffer mix and incubated 10 min. Substrate mix can be added, and reaction mix can be read in kinetic mode for 30 min at Ex480/Em540 and IC₅₀ response curves can be plotted. See, e.g., Chen, et al., Chem Biol., 18(11):1390-1400 (2011).

Exemplary Deubiquitination Assay for ES2 Ub-PCNA IF Activity and Inhibitor Testing.

USP1 is a deubiquitinating enzyme that removes ubiquitin from mono-ubiquitinated Proliferating Cell Nuclear Antigen (Ub-PCNA). The levels of Ub-PCNA in the nucleus of cells can be used to assess the activity of USP1. An immunofluorescence assay can be established to monitor Ub-PCNA levels in the ovarian cancer cell line, ES2.

An assay can be performed by first plating 5000 ES2 cells per well in black 96 well plates (Corning #3904) and then incubating overnight at 37° C. and 5% CO₂. Compounds, resuspended in DMSO, can be added to the cells, to a final DMSO concentration of 0.3%. Plates can be incubated at 37° C. and 5% CO₂ for 3 hours.

Cells can be then fixed and stained by first removing the media from each plate and fixing the cells with −20° C. methanol for 5 minutes at room temperature. Fixation plates can be washed with Tris buffered saline with tween (Boston Bioproducts #IBB-855) 5 times for 5 minutes each. Plates can be blocked for 1 hour with 50 μl of Odyssey blocking buffer (Licor #327-50000) at room temperature with rocking. Block can be removed from all wells and 50 μl of primary antibody was added to each well. Ub-PCNA ab (Cell Signaling Technology #13439) can be diluted at 1:400 in Odyssey blocking buffer. Plates can be sealed and incubated overnight at 4° C. Primary antibodies can be removed from the plates and plates can be washed with Tris buffered saline with tween 5 times for 5 minutes. Plates can be stained for 1 hour at room temperature with rocking with 50 μl of secondary antibodies diluted 1:10,000 in Odyssey blocking buffer at room temperature (anti-rabbit Alexa 488). Antibodies can be removed from the plates and plates can be washed with Tris buffered saline with tween 3 times for 5 minutes. Plates can be washed once with DAPI (Chemometec #910-3012) stain diluted 1:5000 (Stock solution of 500 μg/ml) in tris buffered saline with tween for 5 minutes. DAPI stain can be removed and plates can be washed one additional time for 5 minutes with tris buffered saline with tween. Wash can be removed from the plate and 100 μl tris buffered saline with tween can be added back to all wells, Plates can be sealed with Foil seals or black plate seals and plates can be stored at 4° C. until they can be imaged.

Imaging of the plates can be performed by first locating the nuclei using the DAPI stain. A mask can be created by drawing a circle slightly smaller than each nucleus. Ub-PCNA intensities can be measured for each individual nucleus counted. A histogram of all Ub-PCNA nuclear intensities measured in untreated wells can be generated and a 95% cutoff can be established. This 95% cutoff can be used to determine the number of Positive cells that have Ub-PCNA values higher than the 95% cutoff. The number of positive cells can be expressed as a percentage by dividing by the total number of cells in the well. This percent positive value can be used to graph all data and to determine AC50 values.

### Methods of Use

In some embodiments, the Compounds of the Disclosure can be used to inhibit the activity of a USP1 protein. For example, in some embodiments, a method of inhibiting a USP1 protein comprises contacting the USP1 protein with a Compound of the Disclosure. The contacting can occur in vitro or in vivo.

In some embodiments, the Compounds of the Disclosure can be used to treat a “USP1 protein mediated disorder.” A USP1 protein mediated disorder is any pathological condition in which a USP1 protein is known to play a role. In some embodiments, a USP1 protein mediated disorder is a proliferative disease such as cancer.

Various methods of treating diseases and disorders with the Compounds of the Disclosure are provided herein. Exemplary diseases and disorders that may be treated with the Compounds of the Disclosure include, but are not limited to, cancer.

In some embodiments, methods of treating cancer with Compounds of the Disclosure are provided. Such methods comprise administering to a subject with cancer a therapeutically effective amount of a Compound of the Disclosure.

In some embodiments, the cancer to be treated with a Compound of the Disclosure is selected from a hematological cancer, a lymphatic cancer, and a DNA damage repair pathway deficient cancer. In some embodiments, the cancer to be treated with a Compound of the Disclosure is a cancer that comprises cancer cells with a mutation in a gene encoding p53. In some embodiments, the cancer to be treated with a Compound of the Disclosure is a cancer that comprises cancer cells with a loss of function mutation in a gene encoding p53. In some embodiments, the cancer to be treated with a Compound of the Disclosure is a cancer that comprises cancer cells with a mutation in a gene encoding BRCA1. In some embodiments, the cancer to be treated with a Compound of the Disclosure is a cancer that comprises cancer cells with a mutation in a gene encoding BRCA2. In some embodiments, the cancer to be treated with a Compound of the Disclosure is a cancer that comprises cancer cells with a loss of function mutation in a gene encoding ATM.

In some embodiments, the cancer to be treated with a Compound of the Disclosure is selected from non-small cell lung cancer (NSCLC), osteosarcoma, ovarian cancer, and breast cancer. In some embodiments, the cancer is ovarian cancer or breast cancer. In some embodiments, the cancer is ovarian cancer. In some embodiments, the cancer is breast cancer. In some embodiments, the cancer is a triple negative breast cancer.

In some embodiments, the cancer to be treated with a Compound of the Disclosure is selected from the group consisting of bone cancer, including osteosarcoma and chondrosarcoma; brain cancer, including glioma, glioblastoma, astrocytoma, medulloblastoma, and meningioma; soft tissue cancer, including rhabdoid and sarcoma; kidney cancer; bladder cancer; skin cancer, including melanoma; and lung cancer, including non-small cell lung cancer; colon cancer, uterine cancer; nervous system cancer; head and neck cancer; pancreatic cancer; and cervical cancer.

Various methods of treating cancer with a Compound of the Disclosure are provided herein. In some embodiments, a therapeutically effective amount of Compound of the Disclosure is administered to a subject with cancer, wherein the cancer comprises cancer cells with elevated levels of RAD18. In some embodiments, the elevated levels of RAD18 are elevated RAD18 protein levels. In some embodiments, the elevated levels of RAD18 are elevated RAD18 mRNA levels. In some embodiments, elevated levels of RAD18 (e.g., RAD18 protein and/or RAD18 mRNA) have been detected (e.g., in a cancer sample obtained from the subject) prior to the administration. That is, in some embodiments, a subject's cancer has been tested for RAD18 protein or mRNA prior to beginning treatment with a USP1 inhibitor.

In some embodiments, such methods comprise (a) identifying a cancer in a subject as a USP1 inhibitor-sensitive cancer and then (b) administering a therapeutically effective amount of a Compound of the Disclosure to the subject.

In some embodiments, such methods comprise (a) detecting levels of RAD18 (e.g., RAD18 protein and/or RAD18 mRNA) in cancer cells (e.g., in a cancer sample obtained from the subject) and then (b) administering a therapeutically effective amount of a Compound of the Disclosure to a subject having a cancer comprising cells with elevated levels of RAD18.

In some embodiments, such methods comprise administering to a subject with triple negative breast cancer a therapeutically effective amount of a Compound of the Disclosure.

In some embodiments, a Compound of the Disclosure is used to treat a cancer, wherein the cancer is a homologous-recombination deficient cancer. In some embodiments, a Compound of the Disclosure is used to treat a cancer, wherein the cancer comprises cancer cells with a mutation in a gene encoding p53. In some embodiments, a Compound of the Disclosure is used to treat a cancer, wherein the cancer comprises cancer cells with a loss of function mutation in a gene encoding p53. In some embodiments, a Compound of the Disclosure is used to treat a cancer that does not have a defect in the homologous recombination pathway.

In some embodiments, a Compound of the Disclosure is used to treat a cancer, wherein the cancer is a BRCA1 mutant cancer. In some embodiments, a Compound of the Disclosure is used to treat a cancer, wherein the cancer is a BRCA2 mutant cancer. In some embodiments, a Compound of the Disclosure is used to treat a cancer, wherein the cancer is a BRCA1 mutant cancer and a BRCA2 mutant cancer. In some embodiments, the cancer is not a BRCA1 mutant cancer or a BRCA2 mutant cancer. In some embodiments, the cancer is a BRCA1 deficient cancer. In some embodiments, the cancer is a BRCA2 deficient cancer. In some embodiments, the cancer is a BRCA1 deficient cancer and a BRCA2 mutant cancer.

In some embodiments, a Compound of the Disclosure is used to treat a cancer, wherein the cancer is an ATM mutant cancer. In some embodiments, the cancer is not an ATM mutant cancer. In some embodiments, the cancer is an ATM deficient cancer.

In some embodiments, a Compound of the Disclosure is used to treat a cancer, wherein the cancer is a PARP inhibitor resistant or refractory cancer. In some embodiments, a Compound of the Disclosure is used to treat a cancer, wherein the cancer is a PARP inhibitor resistant or refractory BRCA1-deficient cancer.

In some embodiments, the cancer is a BRCA1 and/or BRCA2 mutant cancer, wherein the cancer comprises cells with elevated levels of RAD18, e.g., wherein the elevated levels of RAD18 are at least as high as the RAD18 protein and/or mRNA levels in ES2 cells or wherein the elevated levels of RAD18 are higher than the RAD18 protein and/or mRNA levels in HEP3B217 cells. In some embodiments, a triple negative breast cancer is a BRCA1 and/or BRCA2 mutant cancer.

In some instances, the cancer is a solid cancer. In some instances, the cancer is a hematological/lymphatic cancer. In some instances, the cancer is a DNA damage repair pathway deficient cancer. In some instances, the cancer is a homolgous-recombination deficient cancer. In some instances, the cancer comprises cancer cells with a mutation in a gene encoding p53. In some instances, the cancer comprises cancer cells with a loss of function mutation in a gene encoding p53. In some instances, the cancer is selected from the group consisting of non-small cell lung cancer (NSCLC), osteosarcoma, ovarian cancer, and breast cancer (including triple negative breast cancer). In some instances, the cancer is ovarian cancer or breast cancer (including triple negative breast cancer). In some instances, the cancer is ovarian cancer. In some instances, the cancer is breast cancer (including triple negative breast cancer.)

In some embodiments, a Compound of the Disclosure is used in combination with one or more additional therapeutic agents to treat cancer. It has been reported that p53 status determines PARP inhibitor sensitization (Sa et al. Genome Biology, (2019) 20:253) and that BRCA1/2 status predicts the efficacy of PARP inhibitors in the clinic (Audeh et al. Lancet (2010) 376 (9737), 245-51). As shown below, p53 mutant cancers and BRCA mutant cancers have increased sensitivity to USP1 inhibitors. Accordingly, in some embodiments, a Compound of the Disclosure is used in combination with a PARP inhibitor to treat cancer.

In some embodiments, provided herein are Compounds of the Disclosure for use as a medicament or for use in preparing a medicament, e.g., for the treatment of cancer. In some embodiments, provided herein are Compounds of the Disclosure for use in a method for the treatment of cancer.

### Pharmaceutical Compositions

Compounds of the Disclosure can be administered to a mammal in the form of a raw chemical without any other components present, or Compounds of the Disclosure can also be administered to a mammal as part of a pharmaceutical composition containing the compound combined with a suitable pharmaceutically acceptable carrier (see, for example, Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). Such a carrier can be selected from pharmaceutically acceptable excipients and auxiliaries. The term “pharmaceutically acceptable carrier” or “pharmaceutically acceptable vehicle” encompasses any of the standard pharmaceutical carriers, solvents, surfactants, or vehicles. Standard pharmaceutical carriers and their formulations are described in Remington's Pharmaceutical Sciences, Mack Publishing Co., Easton, Pa., 19th ed. 1995.

A pharmaceutical composition of the present disclosure may be prepared as liquid suspensions or solutions using a liquid, such as an oil, water, an alcohol, and combinations of these.

A pharmaceutical composition of the present disclosure may be prepared as a sterile injectable, which may be aqueous or oleaginous suspensions. These suspensions may be formulated according to techniques known in the art.

A pharmaceutical composition of the present disclosure may be orally administered in any orally acceptable dosage form including capsules, tablets, aqueous suspensions or solutions.

A pharmaceutical composition of the present disclosure may be administered in the form of suppositories for rectal administration.

A pharmaceutical composition of the present disclosure may also be administered topically, especially when the target of treatment includes areas or organs readily accessible by topical application, including diseases of the eye, the skin, or the lower intestinal tract. Topical application for the lower intestinal tract may be effected in a rectal suppository formulation (see above) or in a suitable enema formulation. Topically-transdermal patches may also be used. For topical applications, the pharmaceutical compositions may be formulated in a suitable ointment, lotion, or cream containing the active component suspended or dissolved in one or more carriers.

A pharmaceutical composition of the present disclosure may also be administered ophthalmically and formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

A pharmaceutical composition of the present disclosure may also be administered by nasal aerosol or inhalation. Such compositions are prepared according to techniques well known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The pharmaceutical compositions to be used for in vivo administration can be sterile. This is readily accomplished by filtration through, e.g., sterile filtration membranes.

Pharmaceutical compositions within the scope of the present disclosure include all compositions where a Compound of the Disclosure is combined with one or more pharmaceutically acceptable carriers. In one embodiment, the Compound of the Disclosure is present in the composition in an amount that is effective to achieve its intended therapeutic purpose.

A pharmaceutical composition of the present disclosure can be administered to any patient that may experience the beneficial effects of a Compound of the Disclosure. Foremost among such patients are mammals, e.g., humans and companion animals, although the disclosure is not intended to be so limited. In one embodiment, the patient is a human. In another embodiment, a pharmaceutical compositions of the present disclosure can be administered to a patient having PARP inhibitor resistant or refractory cancer. In another embodiment, a pharmaceutical compositions of the present disclosure can be administered to a patient having PARP inhibitor resistant or refractory BRCA1-deficient cancer. In another embodiment, a pharmaceutical compositions of the present disclosure can be administered to a patient in combination with a PARP inhibitor.

In another embodiment, the present disclosure provides kits which comprise a Compound of the Disclosure (or a composition comprising a Compound of the Disclosure) packaged in a manner that facilitates their use to practice methods of the present disclosure. In one embodiment, the kit includes a Compound of the Disclosure (or a composition comprising a Compound of the Disclosure) packaged in a container, such as a sealed bottle or vessel, with a label affixed to the container or included in the kit that describes use of the compound or composition to practice the method of the disclosure. In one embodiment, the compound or composition is packaged in a unit dosage form. The kit further can include a device suitable for administering the composition according to the intended route of administration. In some embodiments, the present disclosure provides a kit which comprise a Compound of the Disclosure, or a pharmaceutically acceptable salt or solvate thereof, and instructions for administering the compound, or a pharmaceutically acceptable salt or solvate thereof, to a patient having cancer.

In some embodiments, the present disclosure provides a pharmaceutical composition comprising a Compound of the Disclosure, or a pharmaceutically acceptable salt or solvate thereof, and a pharmaceutically acceptable carrier.

In some embodiments, the present disclosure provides a pharmaceutical composition comprising a compound having Formula I, or a pharmaceutically acceptable salt or solvate thereof, and a pharmaceutically acceptable carrier.

In some embodiments, the present disclosure provides a pharmaceutical composition comprising a compound having Formula II, or a pharmaceutically acceptable salt or solvate thereof, and a pharmaceutically acceptable carrier.

In some embodiments, the present disclosure provides a pharmaceutical composition comprising a compound having Formula III, or a pharmaceutically acceptable salt or solvate thereof, and a pharmaceutically acceptable carrier.

In some embodiments, the present disclosure provides a pharmaceutical composition comprising a compound having Formula IV, or a pharmaceutically acceptable salt or solvate thereof, and a pharmaceutically acceptable carrier.

In some embodiments, the present disclosure provides a pharmaceutical composition comprising a compound having Formula V, or a pharmaceutically acceptable salt or solvate thereof, and a pharmaceutically acceptable carrier.

In some embodiments, the present disclosure provides a pharmaceutical composition comprising a compound having Formula VI, or a pharmaceutically acceptable salt or solvate thereof, and a pharmaceutically acceptable carrier.

In some embodiments, the present disclosure provides a pharmaceutical composition comprising a compound having Formula VII, or a pharmaceutically acceptable salt or solvate thereof, and a pharmaceutically acceptable carrier.

In some embodiments, the present disclosure provides a pharmaceutical composition comprising a compound having Formula VIII, or a pharmaceutically acceptable salt or solvate thereof, and a pharmaceutically acceptable carrier.

In some embodiments, the present disclosure provides a pharmaceutical composition comprising a compound having Formula IX, or a pharmaceutically acceptable salt or solvate thereof, and a pharmaceutically acceptable carrier.

In some embodiments, the present disclosure provides a pharmaceutical composition comprising a compound having Formula X, or a pharmaceutically acceptable salt or solvate thereof, and a pharmaceutically acceptable carrier.

In some embodiments, the present disclosure provides a pharmaceutical composition comprising a compound having Formula XI, or a pharmaceutically acceptable salt or solvate thereof, and a pharmaceutically acceptable carrier.

In some embodiments, the present disclosure provides a pharmaceutical composition comprising a compound having Formula XII, or a pharmaceutically acceptable salt or solvate thereof, and a pharmaceutically acceptable carrier.

In some embodiments, the present disclosure provides a pharmaceutical composition comprising a compound having Formula XIII, or a pharmaceutically acceptable salt or solvate thereof, and a pharmaceutically acceptable carrier.

In some embodiments, the present disclosure provides a pharmaceutical composition comprising a compound having Formula XIV, or a pharmaceutically acceptable salt or solvate thereof, and a pharmaceutically acceptable carrier.

In some embodiments, the present disclosure provides a pharmaceutical composition comprising a compound having Formula XV, or a pharmaceutically acceptable salt or solvate thereof, and a pharmaceutically acceptable carrier.

In some embodiments, the present disclosure provides a pharmaceutical composition comprising a Compound of the Disclosure, or a pharmaceutically acceptable salt or solvate thereof, and a pharmaceutically acceptable carrier, wherein the compound binds to a protein encoded by the USP1 gene.

In some embodiments, the present disclosure provides a pharmaceutical composition comprising a Compound of the Disclosure, or a pharmaceutically acceptable salt or solvate thereof, and a pharmaceutically acceptable carrier, wherein the pharmaceutical composition is for use in treating cancer.

In some embodiments, the present disclosure provides a pharmaceutical composition comprising a Compound of the Disclosure, or a pharmaceutically acceptable salt or solvate thereof, and a pharmaceutically acceptable carrier, wherein the pharmaceutical composition is for the manufacture of a medicament for treatment of cancer.

### EXAMPLES

### General Synthetic Methods

Compounds of the Disclosure are prepared using methods known to those skilled in the art in view of this disclosure, or by the illustrative methods shown in the General Schemes below. In any of the General Schemes, suitable protecting groups can be employed in the synthesis. (See, Wuts, P. G. M.; Greene, T. W., “Greene's Protective Groups in Organic Synthesis”, 4th Ed., J. Wiley & Sons, NY, 2007).

Unless otherwise noted, all reagents were used without further purification. ¹H-NMR spectra were obtained in DMSO-d6 or CD3OD at room temperature on a Bruker AVANCE 400 MHz spectrometer. When more than one conformer was detected, the chemical shifts for the most abundant one is reported. Chemical shifts of ¹H NMR spectra were recorded in parts per million (ppm) on the 6 scale from an internal standard of residual solvent. Splitting patterns are designed as s, singlet; d, doublet; t, triplet; q, quartet; m, multiplet; br, broad. LC-MS and prep-HPLC conditions are described below:

LCMS Shimadzu 3 Minute Method

LC Equipment: Shimadzu LC-20AD series, Binary Pump, Diode Array Detector

LC Column: Agilent Poroshell 120 EC-C_{18, 4.6}x50 mm, 2.7 p.m
Mobile phase: Solvent A: 0.05% formic acid in water (v/v) Solvent B: 0.05% formic acid in MeCN (v/v)

Flow rate: 1.5 mL/min

Acquire time: 3 min
Gradient: hold 15% B for 0.28 min; then 15% to 90% in 2.1 min; then 90% to 100% in 0.01 min; then hold 100% for 0.3 min; then 100% to 15% in 0.01 min; hold at 15% for 0.3 min

Temperature: 25° C.

Wavelength 214 nm, 254 nm

MS Equipment 2020, Quadrupole LC/MS

Ion Source: API-ESI

TIC: 100900 m/z

Drying gas flow: 15 L/min

Nebulizer pressure: 1.5 L/min

Drying gas temp.: 250° C.

Vcap: 4500V

LCMS Shimadzu 5 Minute Method

LC Equipment: Shimadzu LC-20AD series, Binary Pump, Diode Array Detector

LC Column: Agilent Poroshell 120 EC-C_{18, 4.6}x50 mm, 2.7 p.m
Mobile phase: Solvent A: 0.05% formic acid in water (v/v) Solvent B: 0.05% formic acid in MeCN (v/v)

Flow rate: 1.0 mL/min

Acquire time: 5 min
Gradient: hold 15% B for 0.5 min; then 15% to 85% in 3.5 min; then 85% to 100% in 0.01 min; then hold 100% for 0.49 min; then 100% to 15% in 0.01 min; hold at 15% for 0.49 min

Temperature: 25° C.

Wavelength 214 nm, 254 nm

MS Equipment 2020, Quadrupole LC/MS

Ion Source: API-ESI

TIC: 100900 m/z

Drying gas flow: 15 L/min

Nebulizer pressure: 1.5 L/min

Drying gas temp.: 250° C.

Vcap: 4500V

LCMS Agilent 5 Minute Method

LC Equipment: Agilent Technologies 1290 series, Binary Pump, Diode Array Detector

LC Column: Agilent Poroshell 120 EC-C_{18, 4.6}x50 mm, 2.7 p.m
Mobile phase: Solvent A: 0.05% formic acid in water (v/v) Solvent B: 0.05% formic acid in MeCN (v/v)

Flow rate: 1.0 mL/min

Acquire time: 5 min
Gradient: hold 10% B for 0.5 min; then 10% to 90% in 3.5 min; then 90% to 100% in 0.01 min; then hold 100% for 0.49 min; then 100% to 10% in 0.01 min; hold at 10% for 0.49 min

Temperature: 25° C.

Wavelength 214 nm, 254 nm

MS Equipment G6120A, Quadrupole LC/MS

Ion Source: API-ESI

TIC: 701000 m/z

Fragmentor: 70

Drying gas flow: 12 L/min

Nebulizer pressure: 36 psi

Drying gas temp.: 350° C.

Vcap: 3000V

Prepartive HPLC Generic Methods

HPLC Instruments: Shimadzu 20AP

UV detector: SPD-20A

UV wavelength: 214 nm and 254 nm
Conditions 1: Mobile phase A: water Mobile phase B: acetonitrile.
Conditions 2: Mobile phase A: water with 0.1% trifluoroacetic acid Mobile phase B: acetonitrile
Conditions 3: Mobile phase A: water with 0.1% formic acid Mobile phase B: acetonitrile
Conditions 4: Mobile phase A: water with 0.1% ammonium hydroxide Mobile phase B: acetonitrile.

Column: Agilent 10 Prep-C18 250×21.2 mm.

Column temperature: Ambient
LC gradient: 20% to 85% in 20 min; then 85% to 100% in 0.01 min; then hold 100% for 5 min; then 100% to 20% in 0.01 min; hold at 20% for 5 min

LC Flow rate: 20 mL/min binary pump

Chiral Preparative HPLC Generic Methods

HPLC Instruments: Shimadzu 20AP

UV detector: SPD-20A

UV wavelength: 254 nm

Mobile phase A: hexanes

Mobile phase B: ethanol with 0.05% diethylamine

Column: UniChiral OD-5H, 250×21.2 mm

Column temperature: 25° C.

Condition 1: 15% ethanol hold for 30 min.

Condition 2: 20% ethanol hold for 30 min.

Condition 3: 30% ethanol hold for 30 min.

LC Flow rate: 25 mL/min binary pump.

The following abbreviations are used in the text: Chloroform-d (deuterated chloroform); DMSO-d₆ (deuterated dimethylsulfoxide); Methanol-d₄ (deuterated methanol); Boc (tert-butoxycarbonyl); Boc₂O (di-tent-butyl dicarbonate); DMF (N,N-dimethylformamide); NMP (1-methyl-2-pyrrolidinone); DMSO (dimethylsulfoxide); PE (petroleum ether); EDCI (1-ethyl-3-(3-dimethylaminopropyl) carbodiimide); ESI (electrospray atmospheric pressure ionization); TEA (triethylamine); TFA (trifluoroacetic acid); dioxane (1,4-dioxane); THF (tetrahydrofuran); EtOAc (ethyl acetate); g (gram); h (hour); nm (nanometer); ¹H NMR (proton nuclear magnetic resonance); Hz (hertz); LC-MS (liquid chromatography-mass spectrometry); MS (mass spectrometry); mg (milligrams); MHz (megahertz); min (minutes); mL (millilitres), mmol (millimoles); ppm (parts per million); Rt (retention time); RT (room temperature); TLC (thin layer chromatography); v/v (volume/volume); m/z (mass charge ratio); HCl (hydrochloric acid); KOAc (potassium acetate); NaOAc (sodium acetate); Pd/C (palladium on activated carbon); n-BuLi (n-butyllithium); MeI (iodomethane); EtI (iodoethane); LiHMDS (lithium bis(trimethylsilyl)amide); NaHMDS (sodium bis(trimethylsilyl)amide); TMSOI (trimethylsulfoxonium iodide); TMSCHN₂ (trimethylsilyldiazomethane); LDA (lithium diisopropylamide); DIAD (diisopropyl azodicarboxylate); DEAD (diethyl azodicarboxylate); DBAD (di-tent-butyl azodicarboxylate); TMSCF₃ (Trimethyl(trifluoromethyl)silane); X-Phos (2-(dicyclohexylphosphino)-2′,4′,6′-triisopropylbiphenyl); NCS (N-chlorosuccinimide); NBS (N-bromosuccinimide); SEMCl (2-(trimethylsilyl)ethoxymethyl chloride); AIBN (2,2′-azobis(isobutyronitrile)); CO (carbon monoxide); DIPEA (N,N-diisopropylethylamine); TBAF (tetrabutylammonium fluoride); DAST (diethylaminosulfur trifluoride); MW (microwave); Pd(PPh₃)₄ (tetrakis(triphenylphosphine)palladium); Pd₂(dba)₃ (tris(dibenzylideneacetone)dipalladium); Pd(dppf)Cl₂ ([1,1′-Bis(diphenylphosphino)ferrocene]dichloropalladium); NaH (sodium hydride); PPh₃ (triphenylphosphine); HATU (2-(7-aza-1H-benzotriazole-1-yl)-1,1,3,3-tetramethyluronium hexafluorophosphate); TBSC₁ (tert-butyldimethylchlorosilane); ee (enantiomeric excess).

Compound Synthesis: The compounds of the invention may be prepared by methods well known to those skilled in the art, and as described in the synthetic experimental procedures shown below.

### Intermediate 1. 4-cyclopropyl-6-methoxy-5-(4,4,5,5-tetramethyl-1,3,2-dioxaborolan yl)pyrimidine

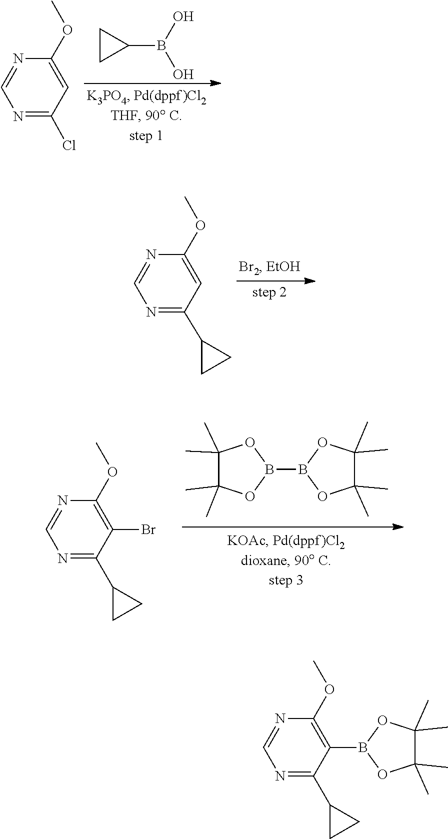

### Step 1. 4-cyclopropyl-6-methoxypyrimidine

A mixture of 4-chloro-6-methoxypyrimidine (5 g, 34.7 mmol), cyclopropylboronic acid (3 g, 34.7 mmol), K₃PO₄ (18.4 g, 80.8 mmol) and Pd(dppf)Cl₂ (2.5 g, 3.5 mmol) in THF (50 mL) was heated at 90° C. in a sealed tube for 16 h. The resulting mixture was filtered through celite, concentrated and purified by silica gel chromatography (eluting with 1/50 EtOAc/PE) to afford 4-cyclopropyl-6-methoxypyrimidine (1.6 g). MS (ESI) m/z 151.1 [M+H]⁺.

### Step 2. 5-bromo-4-cyclopropyl-6-methoxypyrimidine

To a solution of 4-cyclopropyl-6-methoxypyrimidine (1.6 g, 10.7 mmol) in EtOH (10 mL) was added Br₂ (1.87 g, 11.7 mmol) at −20° C. The resulting mixture was slowly warmed to room temperature and stirred at the same temperature for 16 h. Solvent was removed under reduced pressure and the residue dissolved in EtOAc and washed with saturated Na₂CO₃, water and brine. The organic layer was dried over Na₂SO₄ and concentrated to give 5-bromo-4-cyclopropyl-6-methoxypyrimidine (2.3 g). ¹H NMR (400 MHz, Methanol-d₄) δ 8.40 (s, 1H), 4.03 (s, 3H). MS (ESI) m/z 229.0, 231.0 [M+H]⁺.

### Step 3. 4-cyclopropyl-6-methoxy-5-(4,4,5,5-tetramethyl-1,3,2-dioxaborolan-2-yl)pyrimidine

A mixture of 5-bromo-4-cyclopropyl-6-methoxypyrimidine (2.3 g, 10 mmol), 4,4,4′,4′,5,5,5′,5′-octamethyl-2,2′-bi(1,3,2-dioxaborolane) (5.1 g, 20 mmol), KOAc (1.96 g, 20 mmol) and Pd(dppf)Cl₂ (366 mg, 0.5 mmol) in dioxane (20 mL) was heated under an atmosphere of N₂ at 90° C. for 18 h. The resulting mixture was filtered through celite, concentrated and purified on a Biotage Isolera One (C₁₈ column, eluting with 10% to 90% MeCN/H₂O) to afford 4-cyclopropyl-6-methoxy-5-(4,4,5,5-tetramethyl-1,3,2-dioxaborolan-2-yl)pyrimidine (2 g). MS (ESI) m/z 277.2 [M+H]⁺.

### Intermediate 2. 1-isopropyl-4-methyl-5-(4,4,5,5-tetramethyl-1,3,2-dioxaborolan-2-yl)-1H-pyrazole

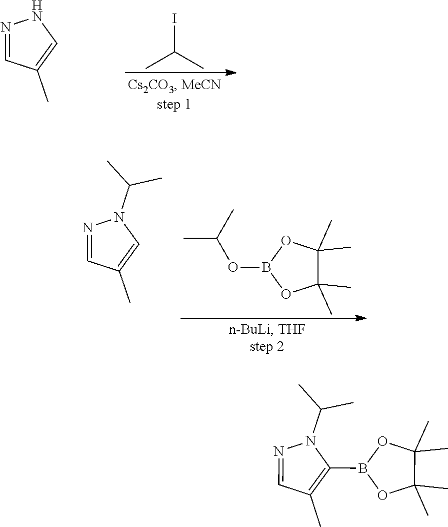

### Step 1. 1-isopropyl-4-methyl-1H-pyrazole

A mixture of 4-methyl-1H-pyrazole (10 g, 121.8 mmol), 2-iodopropane (18.3 mL, 182.7 mmol) and Cs₂CO₃ (79.4 g, 243.6 mmol) in MeCN (70 mL) was heated at 80° C. for 2 h. After cooling to room temperature, the reaction mixture was filtered through celite. The filtrate was concentrated and purified by silica gel chromatography (eluting with 1/10 EtOAc/PE) to afford 1-isopropyl-4-methyl-1H-pyrazole (9.2 g). MS (ESI) m/z 125.1 [M+H]⁺.

### Step 2. 1-isopropyl-4-methyl-5-(4,4,5,5-tetramethyl-1,3,2-dioxaborolan-2-yl)-1H-pyrazole

To a solution of 1-isopropyl-4-methyl-1H-pyrazole (3 g, 24.2 mmol) in dry THF (10 mL) at 0° C. was added n-BuLi (2.5 M in hexane, 11.6 mL, 29.0 mmol) dropwise. After addition, the solution was stirred at room temperature for 1 h before cooling to −78° C. 2-Isopropoxy-4,4,5,5-tetramethyl-1,3,2-dioxaborolane (5.4 g, 29 mol) was added dropwise and the resulting solution was warmed to room temperature and stirred for 2 h then quenched with saturated NH₄Cl solution. The mixture was diluted with EtOAc, washed with water and brine, dried with Na₂SO₄ and concentrated under vacuum to give 1-isopropyl-4-methyl-5-(4,4,5,5-tetramethyl-1,3,2-dioxaborolan-2-yl)-1H-pyrazole (1.5 g). MS (ESI) m/z 251.2 [M+H]⁺.

### Intermediate 3. (1-isopropyl-4-methyl-1H-pyrazol-5-yl)boronic acid

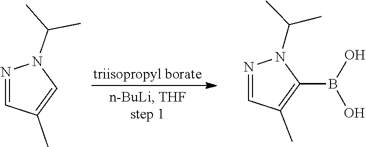

### Step 1. (1-isopropyl-4-methyl-1H-pyrazol-5-yl)boronic acid

To a solution of 1-isopropyl-4-methyl-1H-pyrazole (2 g, 16.1 mmol) in dry THF (15 mL) was added n-BuLi (2.5 M in hexane, 9.7 mL, 24.2 mmol) dropwise over 10 min at −78° C. The resulting solution was stirred at −78° C. for 40 min then triisopropyl borate (9.1 g, 48.3 mmol) was added dropwise over 5 min. The final solution was stirred at −78° C. for 30 min then allowed to warm to room temperature and quenched with saturated NH₄Cl solution. The reaction mixture was directly purified on a Biotage Isolera One (C₁₈ column, eluting with 10% to 90% MeCN/H₂O) to give (1-isopropyl-4-methyl-1H-pyrazol-5-yl)boronic acid (420 mg). MS (ESI) m/z 169.1 [M+H]⁺.

### Intermediate 4. 1-isopropyl-4-methyl-5-(4,4,5,5-tetramethyl-1,3,2-dioxaborolan-2-yl)-1H-pyrazole

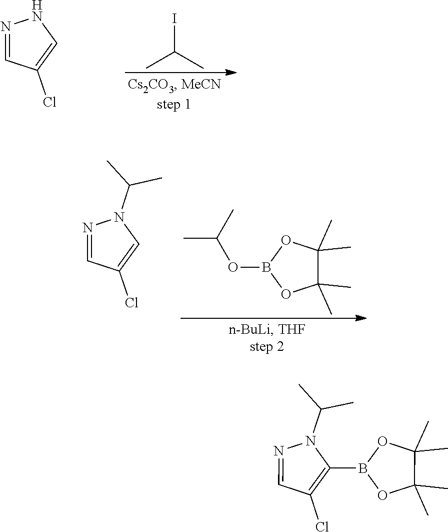

1-isopropyl-4-methyl-5-(4,4,5,5-tetramethyl-1,3,2-dioxaborolan-2-yl)-1H-pyrazole was prepared from 4-chloro-1H-pyrazole following step 1 and step 2 of Intermediate 2. MS (ESI) m/z 271.2 [M+H]⁺.

### Intermediate 5. 4,6-dimethoxy-5-(4,4,5,5-tetramethyl-1,3,2-dioxaborolan-2-yl)pyrimidine

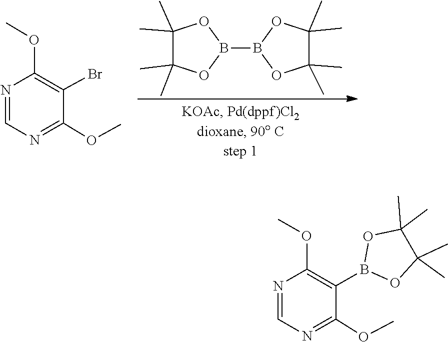

4,6-dimethoxy-5-(4,4,5,5-tetramethyl-1,3,2-dioxaborolan-2-yl)pyrimidine was prepared from 5-bromo-4,6-dimethoxypyrimidine following step 3 of Intermediate 1. MS (ESI) m/z 267.2 [M+H]+.

### Intermediate 6. 4-methoxy-6-methyl-5-(4,4,5,5-tetramethyl-1,3,2-dioxaborolan-2-yl)pyrimidine

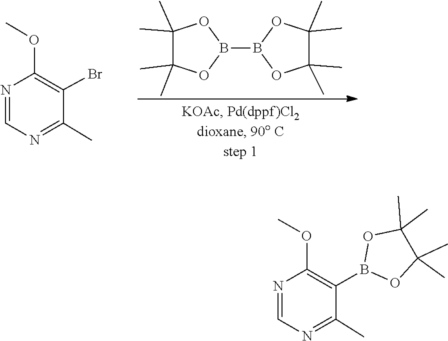

4-methoxy-6-methyl-5-(4,4,5,5-tetramethyl-1,3,2-dioxaborolan-2-yl)pyrimidine was prepared from 5-bromo-4-methoxy-6-methylpyrimidine following step 3 of Intermediate 1. MS (ESI) m/z 251.2 [M+H]⁺.

### Intermediate 7. 2-isopropyl-4-methoxy-3-(4,4,5,5-tetramethyl-1,3,2-dioxaborolan-2-yl)pyridine

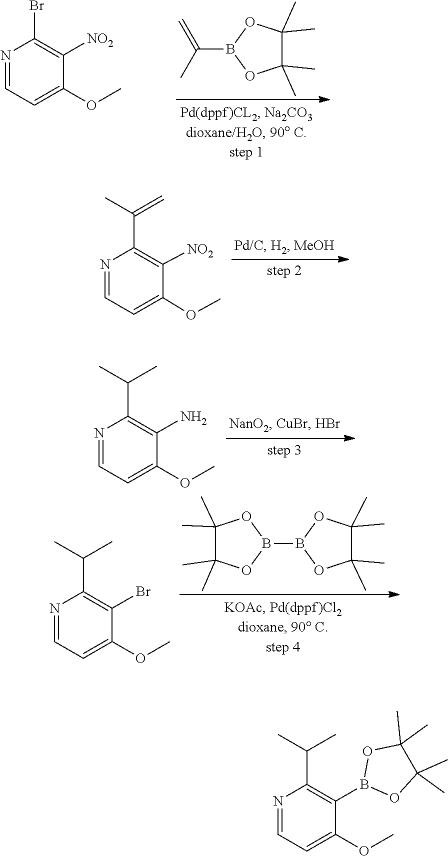

### Step 1. 4-methoxy-3-nitro-2-(prop-1-en-2-yl)pyridine

A mixture of 2-bromo-4-methoxy-3-nitropyridine (2.77 g, 14.7 mmol), 4,4,5,5-tetramethyl-2-(prop-1-en-2-yl)-1,3,2-dioxaborolane (3.7 g, 22.0 mmol), Pd(dppf)Cl₂ (1.07 g, 1.5 mmol) and Na₂CO₃ (3.11 g, 29.4 mmol) in dioxane (40 mL) and H₂O (8 mL) was heated under an atmosphere of N₂ at 90° C. overnight. The resulting mixture was filtered through celite, concentrated and purified by silica gel chromatography (eluting with 1/3 EtOAc/PE) to afford 4-methoxy-3-nitro-2-(prop-1-en-2-yl)pyridine (2.52 g). MS (ESI) m/z 195.2 [M+H]⁺.

### Step 2. 2-isopropyl-4-methoxypyridin-3-amine

To a solution of 4-methoxy-3-nitro-2-(prop-1-en-2-yl)pyridine (2.5 g, 12.8 mmol) in MeOH (25 mL) was added 5% Pd/C (1 g). The resulting mixture was stirred vigorously under an atmosphere of H₂ at room temperature for 2.5 h. The mixture was filtered through celite and concentrated under vacuum to give 2-isopropyl-4-methoxypyridin amine (1.98 g). MS (ESI) m/z 167.2 [M+H]⁺.

### Step 3. 3-bromo-2-isopropyl-4-methoxypyridine

To the solution of 2-isopropyl-4-methoxypyridin-3-amine in HBr aqueous solution (4M, 15 mL) at 0° C. was added NaNO₂ (368 mg, 5.3 mmol) in H₂O (5 mL) dropwise. After addition, the solution was stirred at 0° C. for 1 h then added dropwise into a stirred solution of CuBr₂ in H₂O (10 mL). The final mixture was then stirred at room temperature for 1 h before 4M NaOH aqueous solution was added to adjust the pH to 8-9. The resulting mixture was extracted with EtOAc, dried with Na₂SO₄, concentrated and purified on a Biotage Isolera One (C₁₈ column, eluting with 10% to 90% MeCN/H₂O) to give 3-bromo-2-isopropyl-4-methoxypyridine (630 mg). MS (ESI) m/z 230.1, 232.1 [M+H]⁺.

### Step 4. 2-isopropyl-4-methoxy-3-(4,4,5,5-tetramethyl-1,3,2-dioxaborolan-2-yl)pyridine

2-isopropyl-4-methoxy-3-(4,4,5,5-tetramethyl-1,3,2-dioxaborolan-2-yl)pyridine was prepared from 3-bromo-2-isopropyl-4-methoxypyridine following step 3 of intermediate 1. MS (ESI) m/z 278.2 [M+H]⁺.

### Intermediate 8. 4-chloro-1-ethyl-5-(4,4,5,5-tetramethyl-1,3,2-dioxaborolan-2-yl)-1H-pyrazole

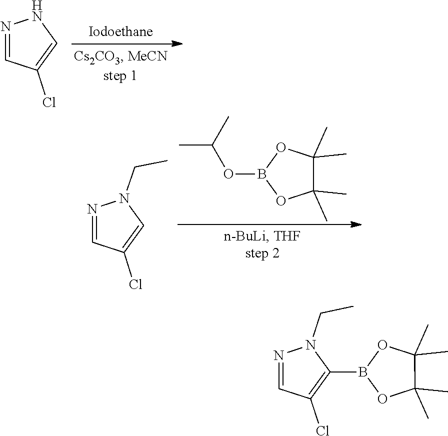

4-chloro-1-ethyl-5-(4,4,5,5-tetramethyl-1,3,2-dioxaborolan-2-yl)-1H-pyrazole was prepared from 4-chloro-1H-pyrazole following step 1 and step 2 of Intermediate 2. MS (ESI) m/z 257.1 [M+H]⁺.

### Intermediate 9. 4-chloro-1-cyclobutyl-5-(4,4,5,5-tetramethyl-1,3,2-dioxaborolan-2-yl)-1H-pyrazole

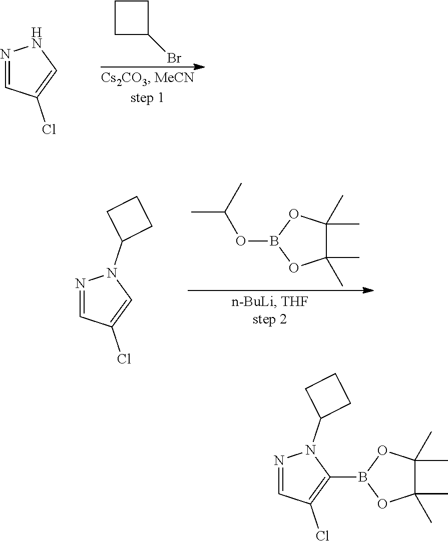

4-chloro-1-cyclobutyl-5-(4,4,5,5-tetramethyl-1,3,2-dioxaborolan-2-yl)-1H-pyrazole was prepared from 4-chloro-1H-pyrazole following step 1 and step 2 of Intermediate 2. MS (ESI) m/z 283.1 [M+H]⁺.

### Intermediate 10. 4-fluoro-1-isopropyl-5-(4,4,5,5-tetramethyl-1,3,2-dioxaborolan-2-yl)-1H-pyrazole

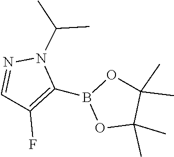 [SMILES: CC(C)n1ncc(F)c1B1OC(C)(C)C(C)(C)O1]

4-fluoro-1-isopropyl-5-(4,4,5,5-tetramethyl-1,3,2-dioxaborolan-2-yl)-1H-pyrazole was prepared from 4-fluoro-1H-pyrazole following step 1 and step 2 of Intermediate 2. MS (ESI) m/z 255.2 [M+H]⁺.

### Intermediate 11. 4-cyclobutyl-6-methoxy-5-(4,4,5,5-tetramethyl-1,3,2-dioxaborolan-2-yl)pyrimidine

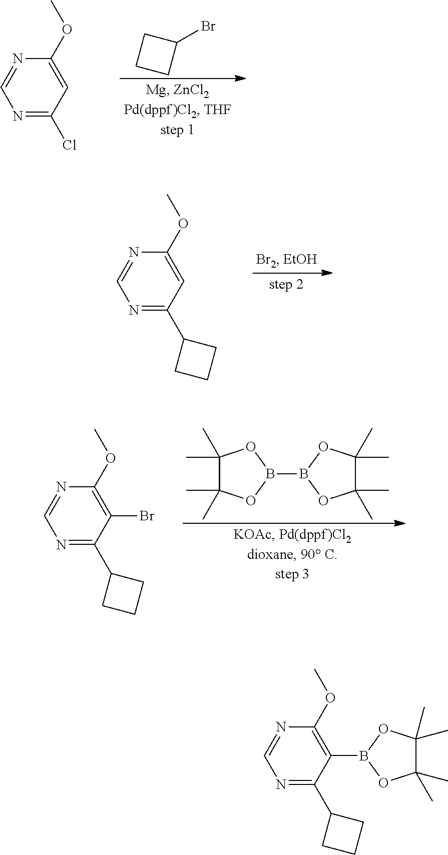

### Step 1. 4-cyclobutyl-6-methoxypyrimidine

To a stirred mixture of magnesium powder (2.5 g, 104 mmol) in dry THF (40 mL) was added drowise bromocyclobutane (9.45 g, 70 mmol) at room temperature to keep the mixture slightly boiling. After the magnesium powder was consumed, ZnCl₂ (14 g, 104 mmol), 4-chloro-6-methoxypyrimidine (1.5 g, 10.4 mmol) and Pd (dppf)C₁₂ (510 mg, 0.7 mmol) were added. The resulting mixture was heated at 70° C. for 12 h then quenched with water (1 mL) and filtered through celite. The filtrate was concentrated and purified on a Biotage Isolera One (C₁₈ column, eluting with 10% to 90% MeCN/H₂O) to give 4-cyclobutyl-6-methoxypyrimidine (1.3 g). MS (ESI) m/z 165.1 [M+H]⁺.

### Step 2 and step 3. 4-cyclobutyl-6-methoxy-5-(4,4,5,5-tetramethyl-1,3,2-dioxaborolan-2-yl)pyrimidine

4-cyclobutyl-6-methoxy-5-(4,4,5,5-tetramethyl-1,3,2-dioxaborolan-2-yl)pyrimidine was prepared from 4-cyclobutyl-6-methoxypyrimidine following step 2 and 3 of intermediate 1. MS (ESI) m/z 291.2 [M+H]⁺.

### Intermediate 12. (1-methylindolin-7-yl)boronic acid

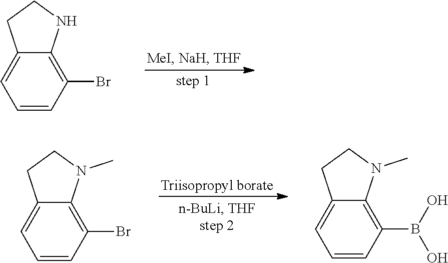

### Step 1. 7-bromo-1-methylindoline

To a solution of 7-bromoindoline (800 mg, 4 mmol) in THF (8 mL) was added NaH (323 mg, 60% in mineral oil, 8 mmol) at 0° C. The mixture was stirred for 30 min then MeI (574 mg, 4 mmol) was added. The resulting mixture was stirred at room temperature for 5 h then quenched with water (20 mL) and extracted with EtOAc (20 mL×3). The combined organic phases were dried over Na₂SO₄ and purified by silica gel chromatography (eluting with 1/30 EtOAc/PE) to afford 7-bromo-1-methylindoline (410 mg). MS (ESI) m/z 212.0, 214.0 [M+H]⁺.

### Step 2. (1-methylindolin-7-yl)boronic acid

To a solution of 7-bromo-1-methylindoline (180 mg, 0.85 mmol) and triisopropyl borate (207.5 mg, 1.1 mmol) in dry THF (3 mL) at −78° C. was added n-BuLi (2.5 M in hexane, 0.44 mL, 1.1 mmol) dropwise. The resulting solution was allowed to warm to room temperature and stirred for 4 h then quenched with saturated NH₄Cl solution. Solvent was removed under vacuum and the crude was directly purified on a Biotage Isolera One (C₁₈ column, eluting with 10% to 90% MeCN/H₂O) to give (1-methylindolin-7-yl)boronic acid (80 mg). MS (ESI) m/z 178.1 [M+H]⁺.

### Intermediate 13. 5-bromo-4-methoxy-6-(1-methylcyclopropyl)pyrimidine

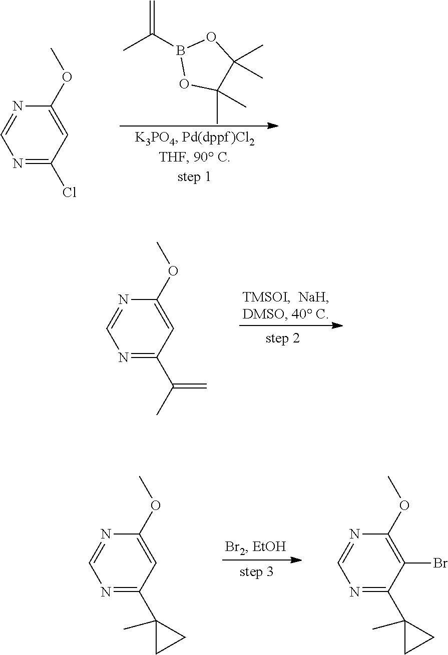

### Step 1. 4-methoxy-6-(prop-1-en-2-yl)pyrimidine

4-methoxy-6-(prop-1-en-2-yl)pyrimidine was prepared from 4-chloro-6-methoxypyrimidine and 4,4,5,5-tetramethyl-2-(prop-1-en-2-yl)-1,3,2-dioxaborolane according to step 1 of Intermediate 1. MS (ESI) m/z 151.1 [M+H]⁺.

### Step 2. 4-methoxy-6-(1-methylcyclopropyl)pyrimidine

To a mixture of trimethylsulfoxonium iodide (9.6 g, 43.7 mmol) in DMSO (15 mL) was added NaH (1.46 g, 60% in mineral oil, 36.4 mmol). The mixture was stirred for at room temperature 30 min under N₂ then a soluton of 4-methoxy-6-(prop-1-en-2-yl)pyrimidine (5.5 g, 36.4 mmol) in DMSO (15 mL) was added. The resulting solution was heated at 40° C. for 5 h then quenched with saturated NH₄Cl solution (50 mL) and extracted with EtOAc (3×50 mL). The organic layers were combined, dried over anhydrous sodium sulfate, concentrated and purified by column chromatography (eluting with a gradient of 0 to 10% EtOAc/PE) to give 4-methoxy-6-(1-methylcyclopropyl)pyrimidine (2.6 g). ¹H NMR (400 MHz, Chloroform-d) δ 8.60 (s, 1H), 6.67 (d, J=0.9 Hz, 1H), 3.95 (s, 3H), 1.44 (s, 3H), 1.33 (t, J=3.2 Hz, 2H), 0.85-0.83 (m, 2H). MS (ESI) m/z 165.1 [M+H]⁺.

### Step 3. 5-bromo-4-methoxy-6-(1-methylcyclopropyl)pyrimidine

5-bromo-4-methoxy-6-(1-methylcyclopropyl)pyrimidine was prepared from 4-methoxy-6-(1-methylcyclopropyl)pyrimidine according to step 2 of Intermediate 1. MS (ESI) m/z 243.0, 245.0 [M+H]⁺.

### Intermediate 14. 4-chloro-5-iodo-1-(tetrahydrofuran-3-yl)-1H-pyrazole

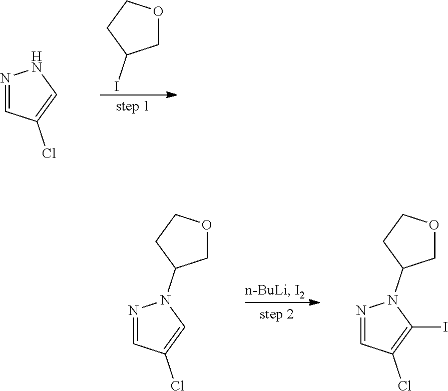 [SMILES: Clc1cn[nH]c1 ; IC1CCOC1 ; Clc1cnn(C2CCOC2)c1 ; Clc1cnn(C2CCOC2)c1I]

### Step 1. 4-chloro-1-(tetrahydrofuran-3-yl)-1H-pyrazole

4-chloro-1-(tetrahydrofuran-3-yl)-1H-pyrazole was prepared from 4-chloro-1H-pyrazole according to step 1 of Intermediate 2. MS (ESI) m/z 173.0 [M+H]⁺.

### Step 2. 4-chloro-5-iodo-1-(tetrahydrofuran-3-yl)-1H-pyrazole

To a solution of 4-chloro-1-(tetrahydrofuran-3-yl)-1H-pyrazole (50 mg, 0.29 mmol) in dry THF (5 mL) at −78° C. was added n-BuLi (2.5 M in hexane, 0.175 mL, 0.44 mmol) dropwise. The solution was stirred at −78° C. for 30 min then a solution of I2 (111 mg, 0.44 mmol) in dry THF (2 mL) was added. The resulting solution was stirred at −78° C. for another 30 min then allowed to warm slowly to room temperature and quenched with saturated NH₄Cl solution. The mixture was diluted with EtOAc, washed with water and brine, dried with Na₂SO₄, concentrated under vacuum and purified by silica gel chromatography (eluting with 1/7 EtOAc/PE) to afford 4-chloro-5-iodo-1-(tetrahydrofuran-3-yl)-1H-pyrazole (63 mg). MS (ESI) m/z 298.9 [M+H]⁺.

### Intermediate 15. 4-chloro-5-iodo-1-(1-methoxypropan-2-yl)-1H-pyrazole

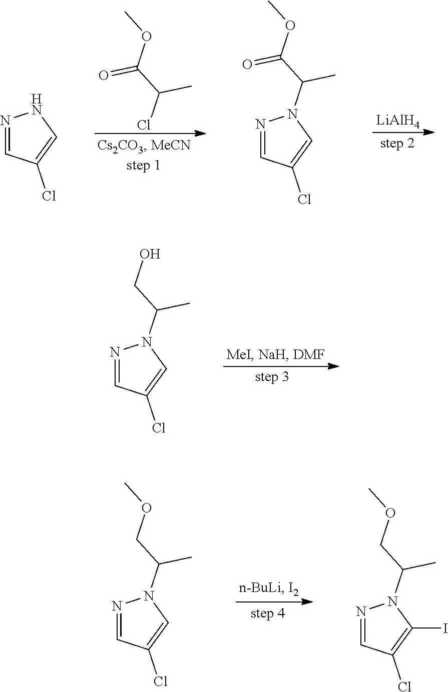

### Step 1. methyl 2-(4-chloro-1H-pyrazol-1-yl)propanoate

methyl 2-(4-chloro-1H-pyrazol-1-yl)propanoate was prepared from 4-chloro-1H-pyrazole according to step 1 of Intermediate 2. MS (ESI) m/z 189.0 [M+H]⁺.

### Step 2. 2-(4-chloro-1H-pyrazol-1-yl)propan-1-ol

To a solution of methyl 2-(4-chloro-1H-pyrazol-1-yl)propanoate(6.3 g, 33.5 mmol) in dry THF (200 mL) was added LiAlH₄ (1.3 g, 33.5 mmol) at 0° C. After addition, the reaction mixture was stirred at room temperature for 30 min. The reaction mixture was quenched with saturated aqueous Na₂CO₃, diluted with DCM (50 mL), filtered through celite and concentrated under vacuo to give 2-(4-chloro-1H-pyrazol-1-yl)propan-1-ol (3.6 g). The crude material was used in the next step without further purification. MS (ESI) m/z 161.3 [M+H]⁺.

### Step 3. 4-chloro-1-(1-methoxypropan-2-yl)-1H-pyrazole

To a solution of 2-(4-chloro-1H-pyrazol-1-yl)propan-1-ol (3.2 g, 20 mmol) in dry DMF (80 mL) was added NaH (60% in mineral oil, 962 mg, 24 mmol) in portions at 0° C. The reaction mixture was stirred for 30 min then iodomethane (8.5 g, 60 mmol) was added. The reaction was stirred at room temperature for 2 h then quenched with water (100 mL). The mixture was extracted with DCM (50 mL×3) and the combined organic layers dried over Na₂SO₄, concentrated under vacuum and purified on a Biotage Isolera One (C₁₈ column, eluting with 10% to 90% MeCN/H₂O) to give 4-chloro-1-(1-methoxypropan-2-yl)-1H-pyrazole(1.8 g). MS (ESI) m/z 175.1 [M+H]⁺.

### Step 4. 4-chloro-5-iodo-1-(1-methoxypropan-2-yl)-1H-pyrazole

4-chloro-5-iodo-1-(1-methoxypropan-2-yl)-1H-pyrazole was prepared from 4-chloro-1-(1-methoxypropan-2-yl)-1H-pyrazole following step 2 of Intermediate 14. MS (ESI) m/z 301.0 [M+H]⁺.

### Intermediate 16. 5-iodo-1-isopropyl-4-methoxy-1H-pyrazol

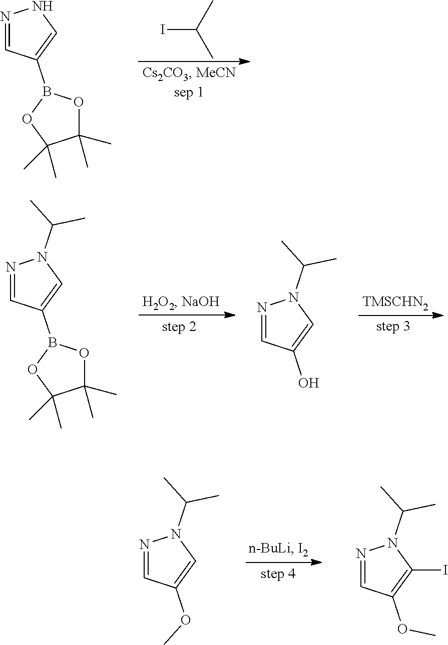

### Step 1. 1-isopropyl-4-(4,4,5,5-tetramethyl-1,3,2-dioxaborolan-2-yl)-1H-pyrazole

1-isopropyl-4-(4,4,5,5-tetramethyl-1,3,2-dioxaborolan-2-yl)-1H-pyrazole was prepared from 4-(4,4,5,5-tetramethyl-1,3,2-dioxaborolan-2-yl)-1H-pyrazole according to step 1 of Intermediate 2. MS (ESI) m/z 237.2 [M+H]⁺.

### Step 2. 1-isopropyl-1H-pyrazol-4-ol

To a solution of 1-isopropyl-4-(4,4,5,5-tetramethyl-1,3,2-dioxaborolan-2-yl)-1H-pyrazole (5.61 g, 23.8 mmol) in THF (50 mL) at 0° C. was added NaOH (2.5 M aqueous solution) and H₂O₂ (30%, 5.3 mL). The resulting solution was allowed to warm to room temperature and stirred for 3 h. The pH was adjusted to 2 with 1.0 M HCl and the resulting solution extracted with EtOAc (50 mL×3), dried over Na₂SO₄, concentrated under vacuum to afford 1-isopropyl-1H-pyrazol-4-ol (4.0 g). MS (ESI) m/z 127.1 [M+H]⁺.

### Step 3. 1-isopropyl-4-methoxy-1H-pyrazole

To a solution of 1-isopropyl-1H-pyrazol-4-ol (1.0 g, 7.94 mmol, 1.0 eq) in MeCN (20 mL) was added TMSCHN₂ (2 M in hexanes, 15.8 mL, 31.8 mmol). The resulting solution was stirred at room temperature for 24 h. The reaction solution was concentrated under vacuum and purified by silica gel chromatography (eluting with 1/7 EtOAc/PE) to afford 1-isopropyl-4-methoxy-1H-pyrazole (415.9 mg). MS (ESI) m/z 141.2 [M+H]⁺.

### Step 4. 5-iodo-1-isopropyl-4-methoxy-1H-pyrazole

5-iodo-1-isopropyl-4-methoxy-1H-pyrazole was prepared from 1-isopropyl-4-methoxy-1H-pyrazole according to step 2 of Intermediate 14. MS (ESI) m/z 267.0 [M+H]⁺.

### Intermediate 17. 4-chloro-5-iodo-1-(oxetan-3-yl)-1H-pyrazole

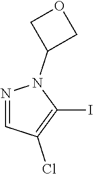 [SMILES: Clc1cnn(C2COC2)c1I]

4-chloro-5-iodo-1-(oxetan-3-yl)-1H-pyrazole was prepared from 4-chloro-1H-pyrazole and 3-iodooxetane according to Intermediate 14. MS (ESI) m/z 284.9 [M+H]⁺.

### Intermediate 18. 5-bromo-1-isopropyl-1H-pyrazole-4-carbonitrile

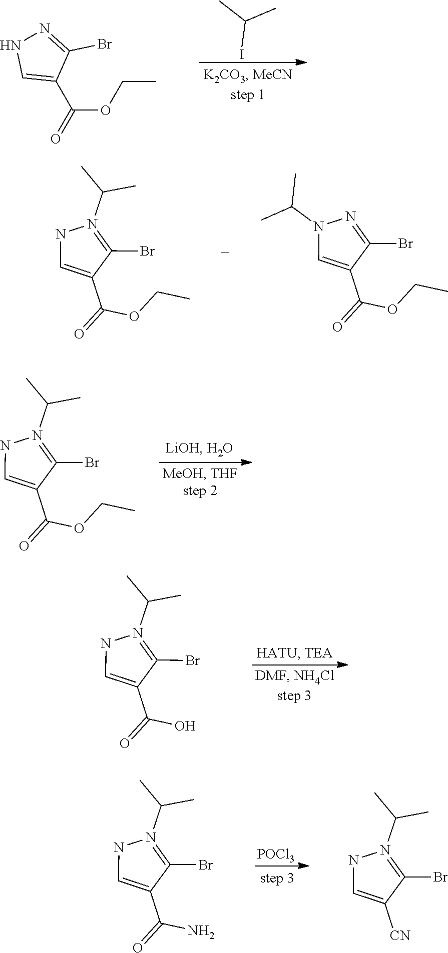

### Step 1. ethyl 5-bromo-1-isopropyl-1H-pyrazole-4-carboxylate and ethyl 3-bromo-1-isopropyl-1H-pyrazole-4-carboxylate

A mixture of ethyl 5-bromo-1H-pyrazole-4-carboxylate (2.0 g, 9.1 mmol), 2-iodopropane (2.3 g, 13.7 mmol, 1.5 eq) and Cs₂CO₃ (5.9 g, 18.2 mmol) in MeCN (50 mL) was heated at 80° C. for 1 h. The mixture was filtered, and the filtrate was concentrated under vacuum. Purification by column chromatography on silica gel (eluting with 1/20 EtOAc/PE) afforded 5-bromo-1-isopropyl-1H-pyrazole-4-carboxylate (650 mg) and ethyl 3-bromo-1-isopropyl-1H-pyrazole-4-carboxylate (936 mg).

ethyl 5-bromo-1-isopropyl-1H-pyrazole-4-carboxylate: ¹H NMR (400 MHz, Chloroform-d) δ 7.93 (s, 1H), 4.79-4.69 (m, 1H), 4.26 (q, J=7.1 Hz, 2H), 1.43 (d, J=6.6 Hz, 6H), 1.30 (t, J=7.0 Hz, 3H).

ethyl 3-bromo-1-isopropyl-1H-pyrazole-4-carboxylate : ¹H NMR (400 MHz, Chloroform-d) δ 7.85 (s, 1H), 4.47-4.37 (m, 1H), 4.26 (q, J=7.1 Hz, 2H), 1.47 (d, J=6.7 Hz, 6H), 1.31 (t, J=7.1 Hz, 3H).

### Step 2. 5-bromo-1-isopropyl-1H-pyrazole-4-carboxylic acid

A mixture of ethyl 5-bromo-1-isopropyl-1H-pyrazole-4-carboxylate (593 mg, 2.27 mmol) and NaOH (276 mg, 6.9 mmol) in EtOH (5 mL) and (5 mL) was heated at 60° C. for 1 h. EtOH was removed under reduced pressure and the pH of the aqueous solution was adjusted to 3 with 2M HCl. The mixture was extracted with EtOAc (20 mL×3), dried over Na₂SO₄, concentrated under vacuum to afford 5-bromo-1-isopropyl-1H-pyrazole-4-carboxylic acid (650 mg), which was used in next step without further purification. MS (ESI) m/z 233.0, 235.0 [M+H]⁺.

### Step 3. 5-bromo-1-isopropyl-1H-pyrazole-4-carboxamide

A mixture of 5-bromo-1-isopropyl-1H-pyrazole-4-carboxylic acid (530 mg, 2.27), NH₄Cl (243 mg, 4.54 mmol), HATU (1.30 g, 3.42 mmol) and triethylamine (688 mg, 6.79 mmol) in DMF (5 mL) was stirred at room temperature for 1 h. The mixture was diluted with water (20 mL) and extracted with EtOAc (30 mL×2). The combine organic phases were washed with brine (20 mL×2), dried over Na₂SO₄, concentrated under vacuum and purified by column chromatography on silica gel (eluting with 1/1 PE/EtOAc) to afford 5-bromo-1-isopropyl-1H-pyrazole-4-carboxamide (170 mg). MS (ESI) m/z 232.0, 234.0 [M+H]⁺.

### Step 4. 5-bromo-1-isopropyl-1H-pyrazole-4-carbonitrile

To a solution of 5-bromo-1-isopropyl-1H-pyrazole-4-carboxamide (160 mg, 0.69 mmol) in DMF (3 mL) was added POCl₃ (317.4 mg, 2.07 mmol) slowly at room temperature. The resulting mixture was stirred at room temperature for 2 h then quenched with saturated NH₄Cl (20 mL) and extracted with EtOAc (20 mL×2). The combine organic phases were washed with brine (20 mL×2), dried over Na₂SO₄ and concentrated under vacuum to afford 5-bromo-1-isopropyl-1H-pyrazole-4-carbonitrile (156 mg) without further purification. MS (ESI) m/z 255.0, 257.0 [M+H]⁺.

### Intermediate 19. 4-(tert-butyl)-5-iodo-1-methyl-1H-pyrazole

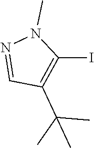

4-(tert-butyl)-5-iodo-1-methyl-1H-pyrazole was prepared from 4-(tert-butyl)-1H-pyrazole according to Intermediate 14. MS (ESI) m/z 265.0 [M+H]⁺.

### Intermediate 20. 1-isopropyl-4-methoxy-5-(4,4,5,5-tetramethyl-1,3,2-dioxaborolan-2-yl)-1H-pyrazole

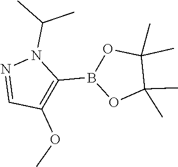 [SMILES: COc1cnn(C(C)C)c1B1OC(C)(C)C(C)(C)O1]

1-isopropyl-4-methoxy-5-(4,4,5,5-tetramethyl-1,3,2-dioxaborolan-2-yl)-1H-pyrazole was prepared from 1-isopropyl-4-methoxy-1H-pyrazole (prepared from step 3 of Intermediate 16) following step 2 of Intermediate 2. MS (ESI) m/z 267.2 [M+H]⁺.

### Intermediate 21. 5-iodo-4-isopropyl-1-methyl-1H-pyrazole

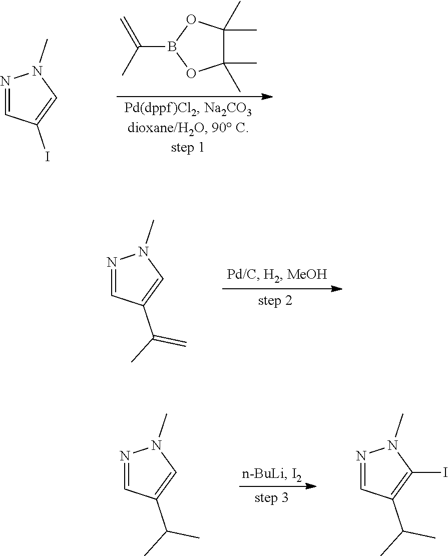

### Step 1. 1-methyl-4-(prop-1-en-2-yl)-1H-pyrazole

1-methyl-4-(prop-1-en-2-yl)-1H-pyrazole was prepared from 4-iodo-1-methyl-1H-pyrazole according to step 1 of intermediate 7. MS (ESI) m/z 123.1 [M+H]⁺.

### Step 2. 4-isopropyl-1-methyl-1H-pyrazole

4-isopropyl-1-methyl-1H-pyrazole was prepared from 1-methyl-4-(prop-1-en-2-yl)-1H-pyrazole according to step 2 of intermediate 7. MS (ESI) m/z 125.1 [M+H]⁺.

### Step 3. 5-iodo-4-isopropyl-1-methyl-1H-pyrazole

5-iodo-4-isopropyl-1-methyl-1H-pyrazole was prepared from 4-isopropyl-1-methyl-1H-pyrazole according to step 2 of Intermediate 14. MS (ESI) m/z 251.0 [M+H]⁺.

### Intermediate 22. 4-chloro-5-iodo-1-(1-(methoxymethyl)cyclopropyl)-1H-pyrazole

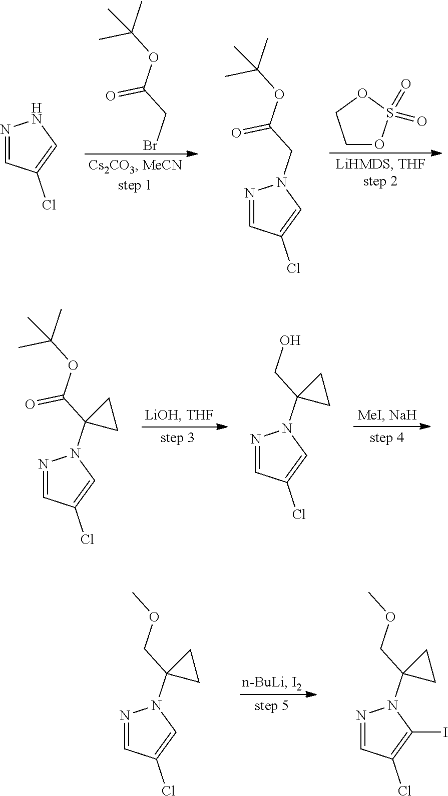

### Step 1. tert-butyl 2-(4-chloro-1H-pyrazol-1-yl)acetate

tert-butyl 2-(4-chloro-1H-pyrazol-1-yl)acetate was prepared from 4-chloro-1H-pyrazole and tert-butyl 2-bromoacetate following step 1 of intermediate 2. MS (ESI) m/z 217.1 [M+H]⁺.

### Step 2. tert-butyl 1-(4-chloro-1H-pyrazol-1-yl)cyclopropane-1-carboxylate

To a solution of tert-butyl 2-(4-chloro-1H-pyrazol-1-yl)acetate (1.0 g, 4.63 mmol) in THF (10 mL) was added LiHMDS (1 M in THF, 4.6 mL) at −78° C. The mixture was stirred at −78° C. for 30 min then 1,3,2-dioxathiolane 2,2-dioxide (574 mg, 4.63 mmol) was added. The reaction was allowed to warm to room temperature and stirred for 2 h. The mixture was quenched with water, concentrated and purified with a Biotage Isolera One (C₁₈ column, eluting with 10% to 90% MeCN/H₂O) to afford tert-butyl 1-(4-chloro-1H-pyrazol-1-yl)cyclopropane-1-carboxylate (171 mg). MS (ESI) m/z 243.1 [M+H]⁺.

### Step 3 to step 5. 4-chloro-5-iodo-1-(1-(methoxymethyl)cyclopropyl)-1H-pyrazole

(1-(4-chloro-1H-pyrazol-1-yl)cyclopropyl)methanol was prepared from tert-butyl 1-(4-chloro-1H-pyrazol-1-yl) cyclopropane-1-carboxylate following to step 2 to step 4 of Intermediate 15. MS (ESI) m/z 313.0 [M+H]⁺.

### Intermediate 23. 4-isopropyl-6-methoxy-5-(4,4,5,5-tetramethyl-1,3,2-dioxaborolan-2-yl)pyrimidine

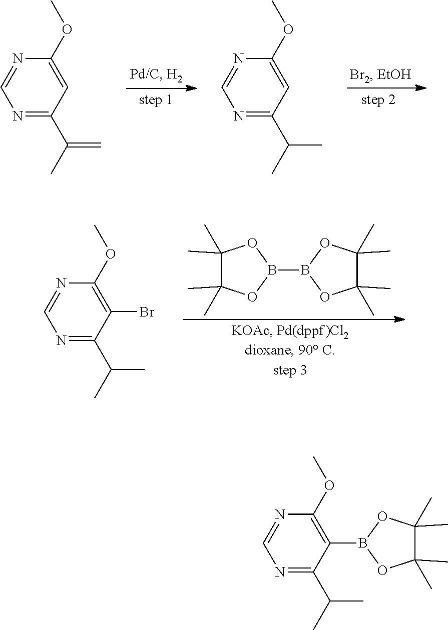

### Step 1. 4-isopropyl-6-methoxypyrimidine

4-isopropyl-6-methoxypyrimidine was prepared from 4-methoxy-6-(prop-1-en-2-yl)pyrimidine following step 2 of intermediate 7. MS (ESI) m/z 153.1 [M+H]⁺.

### Step 2 and step 3. 4-isopropyl-6-methoxy-5-(4,4,5,5-tetramethyl-1,3,2-dioxaborolan-2-yl)pyrimidine

4-isopropyl-6-methoxy-5-(4,4,5,5-tetramethyl-1,3,2-dioxaborolan-2-yl)pyrimidine was prepared from 4-isopropyl-6-methoxypyrimidine following step 2 and step 3 of Intermediate 1. MS (ESI) m/z 279.2 [M+H]⁺.

### Intermediate 24. 4-cyclopropyl-5-iodo-1-isopropyl-1H-pyrazole

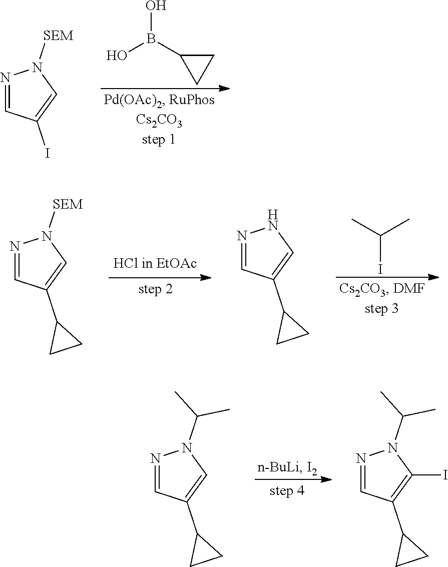

### Step 1. 4-cyclopropyl-1((2-(trimethylsilyl)ethoxy)methyl)-1H-pyrazole

A mixture of 4-iodo-1((2-(trimethylsilyl)ethoxy)methyl)-1H-pyrazole (3.96 g, 12.2 mmol), cyclopropylboronic acid (1.98 g, 13.4 mmol), Pd(OAc)₂ (139 mg, 0.61 mmol), RuPhos (574 mg, 1.22 mmol) and Cs₂CO₃ (12 g, 36.6 mmol) in toluene (36 mL) and H₂O (3.6 mL) was heated under an atmosphere of N₂ at 95° C. overnight. The reaction mixture was filtered through celite, concentrated under vacuum then purified by silica gel chromatography (eluting with 1/50 EtOAc/PE) to afford 4-cyclopropyl-1-((2-(trimethylsilyl)ethoxy)methyl)-1H-pyrazole (4.62 g). MS (ESI) m/z 239.2 [M+H]⁺.

### Step 2. 4-cyclopropyl-1H-pyrazole

A mixture of 4-cyclopropyl-1((2-(trimethylsilyl)ethoxy)methyl)-1H-pyrazole (1.0 g, 4.2 mmol) in HCl-EtOAc solution (4M, 10 mL) was heated at 40° C. for 4 h. Solvent was removed under vacuum to give crude 4-cyclopropyl-1H-pyrazole (450 mg) which was used directly in next step without further purification. MS (ESI) m/z 109.1 [M+H]⁺.

### Step 3 and step 4. 4-cyclopropyl-5-iodo-1-isopropyl-1H-pyrazole

4-cyclopropyl-5-iodo-1-isopropyl-1H-pyrazole was prepared from 4-cyclopropyl-1H-pyrazole following Intermediate 14. MS (ESI) m/z 277.0 [M+H]⁺.

### Intermediate 25. 3-chloro-2-cyclopropyl-4-methoxypyridine

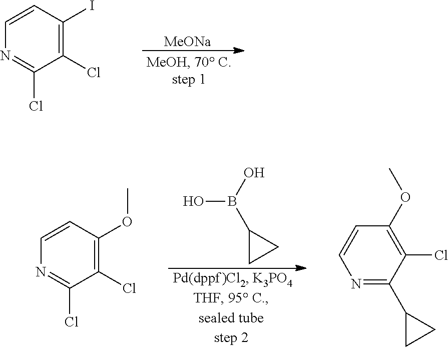

### Step 1. 2,3-dichloro-4-methoxypyridine

To a solution of 2,3-dichloro-4-iodopyridine (10 g, 36.6 mmol) in MeOH (100 mL) was added sodium methoxide (1.98 g, 36.6 mmol) slowly at room temperature. The resulting mixture was heated at 70° C. overnight. The mixture was concentrated and purified with a Biotage Isolera One (C₁₈ column, eluting with 10% to 90% MeCN/H₂O) to afford 2,3-dichloro-4-methoxypyridine (1.82 g). MS (ESI) m/z 178.0 [M+H]⁺.

### Step 2. 3-chloro-2-cyclopropyl-4-methoxypyridine

A mixture of 2,3-dichloro-4-methoxypyridine (1.82 g, 10.3 mmol), cyclopropylboronic acid (1.15 g, 13.4 mmol), Pd(dppf)Cl₂ (753.6 mg, 1.03 mmol) and K₃PO₄ (4.37 g, 20.6 mmol) in THF (20 mL) was heated at 95° C. overnight under an atmosphere of N₂. The mixture was filtered through celite. The filtrate was concentrated and purified with a Biotage Isolera One (C₁₈ column, eluting with 10% to 90% MeCN/H₂O) to afford 3-chloro-2-cyclopropyl-4-methoxypyridine (1.40 g). MS (ESI) m/z 184.0 [M+H]⁺.

### Intermediate 26. 4-cyclopropyl-2-methoxy-3-(4,4,5,5-tetramethyl-1,3,2-dioxaborolan-2-yl)pyridine

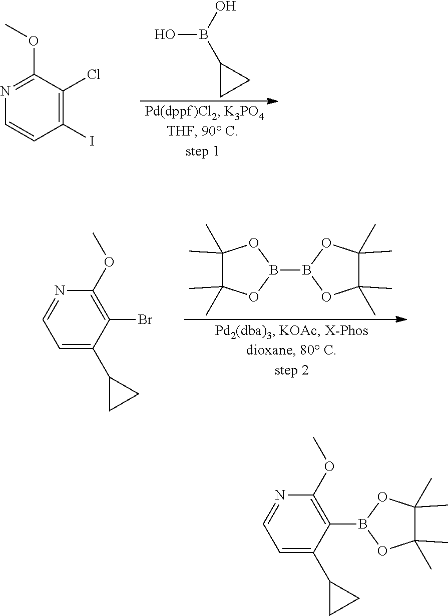

### Step 1. 3-chloro-4-cyclopropyl-2-methoxypyridine

A mixture of 3-chloro-4-iodo-2-methoxypyridine (500 mg, 1.9 mmol), cyclopropylboronic acid (212 mg, 2.42 mmol), Pd(dppf)Cl₂ (139 mg, 0.19 mmol) and K₃PO₄ (806 mg, 3.8 mmol) in THF (8 mL) was heated at 95° C. overnight in a sealed tube. The mixture was filtered through celite. The filtrate was concentrated and purified with a Biotage Isolera One (C₁₈ column, eluting with 10% to 90% MeCN/H₂O) to afford 3-chloro-4-cyclopropyl-2-methoxypyridine (250 mg). MS (ESI) m/z 184.0 [M+H]⁺.

### Step 2. 4-cyclopropyl-2-methoxy-3-(4,4,5,5-tetramethyl-1,3,2-dioxaborolan-2-yl)pyridine

A mixture of 3-chloro-4-cyclopropyl-2-methoxypyridine (330 mg, 1.8 mmol), 4,4,4′,4′,5,5,5′,5′-octamethyl-2,2′-bi(1,3,2-dioxaborolane) (1.4 g, 5.4 mmol), KOAc (529 mg, 5.4 mmol), Pd₂(dba)₃ (165 mg, 0.18 mmol) and X-Phos (172 mg, 0.36 mmol) in dioxane (25 mL) was heated at 80° C. for 18 h under an atmosphere of N₂. The resulting mixture was filtered through celite, concentrated and purified on a Biotage Isolera One (C₁₈ column, eluting with 10% to 90% MeCN/H₂O) to afford 4-cyclopropyl-2-methoxy-3-(4,4,5,5-tetramethyl-1,3,2-dioxaborolan-2-yl)pyridine (394 mg). MS (ESI) m/z 276.2 [M+H]⁺.

### Intermediate 27. (R)-4-chloro-5-iodo-1-(1-methoxypropan-2-yl)-1H-pyrazole

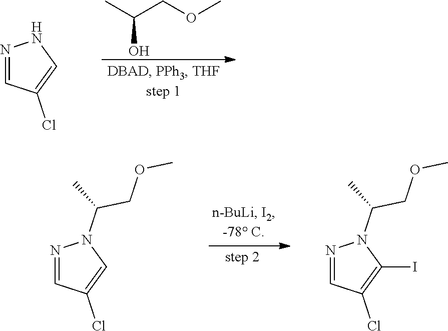

### Step 1. (R)-4-chloro-1-(1-methoxypropan-2-yl)-1H-pyrazole

To a solution of 4-chloro-1H-pyrazole (1 g, 10 mmol), (S)-1-methoxypropan-2-ol (900 mg, 10 mmol) and PPh₃ (3.14 g, 12 mmol) in THF (20 mL) was added DBAD (2.76 g, 12 mmol) slowly at room temperature. The resulting mixture was stirred at room temperature for 8 h. The solution was concentrated under vacuum and purified on a Biotage Isolera One (C₁₈ column, eluting with 10% to 90% MeCN/H₂O) to afford (R)-4-chloro-1-(1-methoxypropan-2-yl)-1H-pyrazole (1.35 g). ¹H NMR (400 MHz, DMSO-d₆) δ 7.97-7.92 (m, 1H), 7.50 (s, 1H), 4.49 (td, J=7.5, 4.6 Hz, 1H), 3.59 (dd, J=10.2, 7.8 Hz, 1H), 3.52 (d, J=4.8 Hz, 2H), 3.18 (s, 3H), 1.34 (d, J=6.9 Hz, 3H). MS (ESI) m/z 175.1 [M+H]⁺.

### Step 2. (R)-4-chloro-5-iodo-1-(1-methoxypropan-2-yl)-1H-pyrazole

(R)-4-chloro-5-iodo-1-(1-methoxypropan-2-yl)-1H-pyrazole was prepared from (R)-4-chloro-1-(1-methoxypropan-2-yl)-1H-pyrazole according to step 2 of Intermediate 14. MS (ESI) m/z 301.0 [M+H]⁺.

### Intermediate 28. (S)-4-chloro-5-iodo-1-(1-methoxypropan-2-yl)-1H-pyrazole

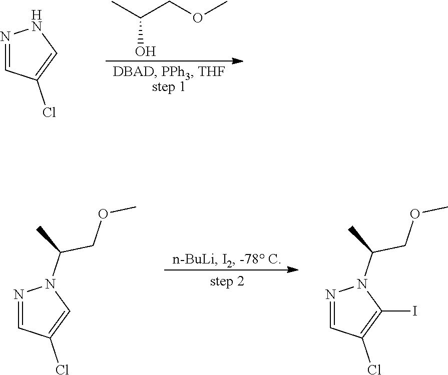

(S)-4-chloro-5-iodo-1-(1-methoxypropan-2-yl)-1H-pyrazole was prepared from 4-chloro-1H-pyrazole and (R)-1-methoxypropan-2-ol according to Intermediate 27. MS (ESI) m/z 301.0 [M+H]⁺.

### Intermediate 29. 4-chloro-5-iodo-1-(1-methoxy-2-methylpropan-2-yl)-1H-pyrazole

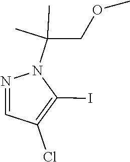

4-chloro-5-iodo-1-(1-methoxy-2-methylpropan-2-yl)-1H-pyrazole was prepared from 4-chloro-1H-pyrazole and methyl 2-bromo-2-methylpropanoate following Intermediate 15. MS (ESI) m/z 315.0 [M+H]⁺.

### Intermediate 30. 2-(5-bromo-4-chloro-1H-pyrazol-1-yl)ethan-1-ol

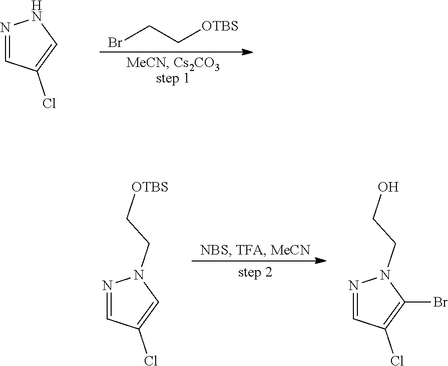

### Step 1. 1-(2-((tert-butyldimethylsilyl)oxy)ethyl)-4-chloro-1H-pyrazole

1-(2-((tert-butyldimethylsilyl)oxy)ethyl)-4-chloro-1H-pyrazole was synthesized from 4-chloro-1H-pyrazole and (2-bromoethoxy)(tert-butyl)dimethylsilane following step 1 of Intermediate 2. MS (ESI) m/z 261.1 [M+H]⁺.

### Step 2. 2-(5-bromo-4-chloro-1H-pyrazol-1-yl)ethan-1-ol

To a solution of 1-(2-((tert-butyldimethylsilyl)oxy)ethyl)-4-chloro-1H-pyrazole (3.8 g, 14.57 mmol) in MeCN (50 mL) was added NBS (2.6g, 14.57 mmol) and TFA (1.7 g, 14.57 mmol). The mixture was stirred at room temperature for 12 h. The resulting mixture was concentrated under vacuum and purified by a Biotage Isolera One (C₁₈ column, eluting with 10% to 95% MeCN/H₂O) and prep-HPLC (condition 2) to afford 2-(5-bromo-4-chloro-1H-pyrazol-1-yl)ethan-1-ol (400 mg). MS (ESI) m/z 224.9, 226.9 [M+H]⁺.

### Intermediate 31. 4-cyclopropyl-6-(difluoromethoxy)-5-(4,4,5,5-tetramethyl-1,3,2-dioxaborolan-2-yl)pyrimidine

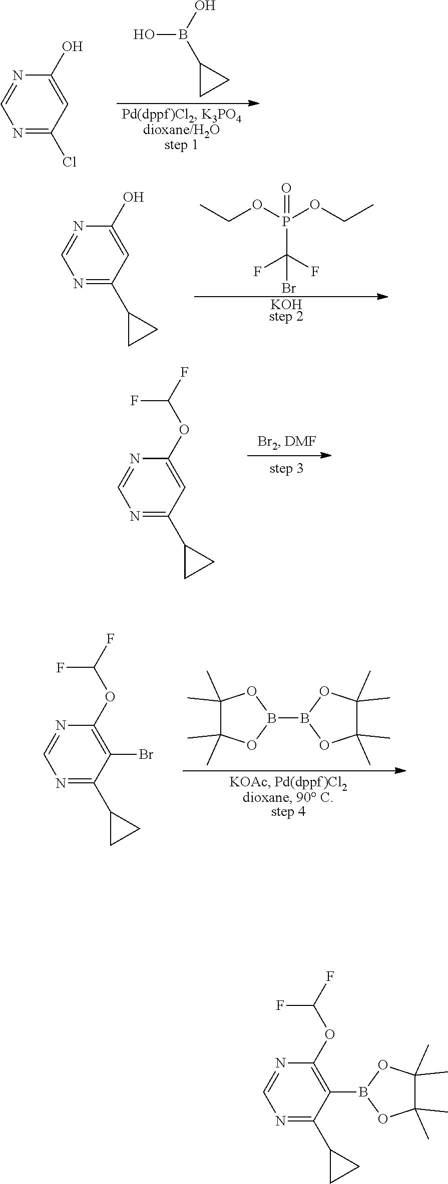

### Step 1. 6-cyclopropylpyrimidin-4-ol

A mixture of 6-chloropyrimidin-4-ol (3 g, 23 mmol), cyclopropylboronic acid (9.9 g, 115 mmol), Pd(dppf)Cl₂ (1.7 g, 2.3 mmol) and K₃PO₄ (10 g, 46 mmol) in dioxane (60 mL) and H₂O (6 mL) was heated at 110° C. overnight under N₂ in a sealed tube. The mixture was filtered through celite. The filtrate was concentrated and purified by silica gel chromatography (eluting with 1/30 MeOH/DCM) to afford 6-cyclopropylpyrimidin-4-ol (1.1 g). MS (ESI) m/z 137.1 [M+H]⁺.

### Step 2. 4-cyclopropyl-6-(difluoromethoxy)pyrimidine

To a solution of 6-cyclopropylpyrimidin-4-ol (1.1 g, 8.1 mmol) in MeCN (11 mL) was added KOH (9 g, 0.16 mol) in H₂O (11 mL) dropwise at 0° C. The solution was stirred at room temperature for 30 min then cooled to −10° C. and diethyl (bromodifluoromethyl)phosphonate (4.3 g, 16.2 mmol) was added. The resulting solution was allowed to warm to room temperature and stirred for 4 h. The mixture was extracted with EtOAc. The organic layer was dried over anhydrous Na₂SO₄, concentrated under vacuum and purified by silica gel chromatography (eluting with 1/40 EtOAc/PE) to afford 4-cyclopropyl-6-(difluoromethoxy)pyrimidine (430 mg). ¹H NMR (400 MHz, Chloroform-d) δ 8.61 (s, 1H), 7.49 (s, 1H), 6.72 (s, 1H), 2.02-1.93 (m, 1H), 1.16 (dt, J=5.1, 2.9 Hz, 2H), 1.10 (dt, J=8.0, 2.9 Hz, 2H). MS (ESI) m/z 187.1 [M+H]⁺.

### Step 3: 5-bromo-4-cyclopropyl-6-(difluoromethoxy)pyrimidine

To a solution of 4-cyclopropyl-6-(difluoromethoxy)pyrimidine (430 mg, 2.3 mmol) in DMF (4 mL) was added Br₂ (1.8 g, 11.6 mol) dropwise at 0° C. The resulting mixture was heated at 50° C. for 2 h. The mixture was diluted with water (20 mL) and extracted with EtOAc (20 mL×2). The combine organic phases were washed with brine, dried over Na₂SO₄, concentrated under vacuum and purified by silica gel chromatography (eluting with 1/40 EtOAc/PE) to 5-bromo-4-cyclopropyl-6-(difluoromethoxy)pyrimidine (275 mg). MS (ESI) m/z 265.0, 267.0 [M+H]⁺.

### Step 4: 4-cyclopropyl-6-(difluoromethoxy)-5-(4,4,5,5-tetramethyl-1,3,2-dioxaborolan-2-yl)pyrimidine

A mixture of 5-bromo-4-cyclopropyl-6-(difluoromethoxy)pyrimidine (1.5 g, 5.6 mmol), 4,4,4′,4′,5,5,5′,5′-octamethyl-2,2′-bi(1,3,2-dioxaborolane) (4.3 g, 16.8 mmol), Pd(dppf)Cl₂ (410 mg, 0.56 mmol) and KOAc (1.6 g, 16.8 mmol) in dioxane (15 mL) was heated at 90° C. overnight under an atmosphere of N₂. The mixture was filtered through celite. The filtrate was concentrated and purified by silica gel chromatography (eluting with 1/20 MeOH/DCM) and a Biotage Isolera One (C₁₈ column, eluting with 10% to 95% MeCN/H₂O) to afford 4-cyclopropyl-6-(difluoromethoxy)-5-(4,4,5,5-tetramethyl-1,3,2-dioxaborolan-2-yl)pyrimidine (950 mg). MS (ESI) m/z 313.1 [M+H]⁺.

### Intermediate 32. 2-bromo-1-ethyl-4-(trifluoromethyl)-1H-imidazole

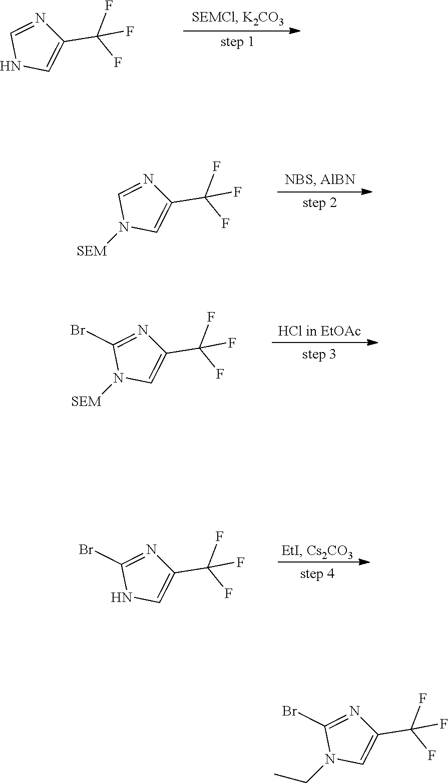

### Step 1. 4-(trifluoromethyl)-1-((2-(trimethylsilyl)ethoxy)methyl)-1H-imidazole

A mixture of 4-(trifluoromethyl)-1H-imidazole (6 g, 44.1 mmol), 2-(trimethylsilyl)ethoxymethyl chloride (8 g, 48.5 mmol) and K₂CO₃ (12 g, 88.2 mmol) in MeCN (60 mL) was stirred at room temperature for 6 h. The resulting mixture was filtered through celite, concentrated and purified on a Biotage Isolera One (C₁₈ column, eluting with 10% to 90% MeCN/H₂O) to afford 4-(trifluoromethyl)-1-((2-(trimethylsilyl)ethoxy)methyl)-1H-imidazole (7.8 g). MS (ESI) m/z 267.1 [M+H]⁺.

### Step 2. 2-bromo-4-(trifluoromethyl)-1-((2-(trimethylsilyl)ethoxy)methyl)-1H-imidazole

A mixture of 4-(trifluoromethyl)-1-((2-(trimethylsilyl)ethoxy)methyl)-1H-imidazole (4.12 g, 15.6 mmol), NBS (3.6 g, 20.3 mmol) and AIBN (512 mg, 3.12 mmol) in CCl₄ (60 mL) was heated at 60° C. for 16 h. The resulting mixture was filtered through celite, concentrated and purified on a Biotage Isolera One (C₁₈ column, eluting with 10% to 90% MeCN/H₂O, containing 0.1% formic acid) to afford 2-bromo-4-(trifluoromethyl)-1-((2-(trimethylsilyl)ethoxy)methyl)-1H-imidazole (1.8 g). MS (ESI) m/z 345.0, 347.0 [M+H]⁺.

### Step 3. 2-bromo-4-(trifluoromethyl)-1H-imidazole

A solution of 2-bromo-4-(trifluoromethyl)-1-((2-(trimethylsilyl)ethoxy)methyl)-1H-imidazole (344 mg, 1.0 mmol) in HCl EtOAc solution (4M, 10 mL) was stirred at room temperature for 2 h. The resulting mixture was concentrated and dried under vacuum to afford 2-bromo-4-(trifluoromethyl)-1H-imidazole (160 mg). MS (ESI) m/z 215.0, 217.0 [M+H]⁺.

### Step 4. 2-bromo-1-ethyl-4-(trifluoromethyl)-1H-imidazole

To a mixture of 2-bromo-4-(trifluoromethyl)-1H-imidazole (363 mg, 1.7 mmol) and Cs₂CO₃(1.7 g, 5.1 mmol) in MeCN (20mL) was added iodoethane (312 mg, 2.0 mmol). The resulting mixture was stirred at room temperature for 2 h then filtered through celite. The filtrate was concentrated and purified on a Biotage Isolera One (C₁₈ column, eluting with 10% to 90% MeCN/H₂O) to afford 2-bromo-1-ethyl-4-(trifluoromethyl)-1H-imidazole (120 mg). MS (ESI) m/z 243.0, 245.0 [M+H]⁺.

### Intermediate 33. 2-bromo-3-(2,2-difluoroethoxy)pyridine

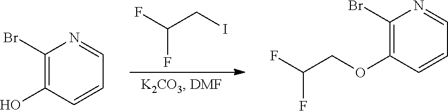

A mixture of 2-bromopyridin-3-ol (500 mg, 2.87 mmol), 1,1-difluoro-2-iodoethane (826 mg, 4.31 mmol) and K₂CO₃ (792 mg, 5.74 mmol) in DMF (5 mL) was stirred at room temperature overnight. The resulting mixture was diluted with water (50 mL) and extracted with EtOAc (50 mL×2). The combine organic phases were washed with brine, dried over Na₂SO₄, concentrated under vacuum and purified by silica gel chromatography (eluting with 1/10 EtOAc/PE) to give 2-bromo-3-(2,2-difluoroethoxy)pyridine (610 mg). MS (ESI) m/z 238.0, 240.0 [M+H]⁺.

### Intermediate BB-1. 2-(4-(bromomethyl)phenyl)-1-methyl-4-(trifluoromethyl)-1H-imidazole

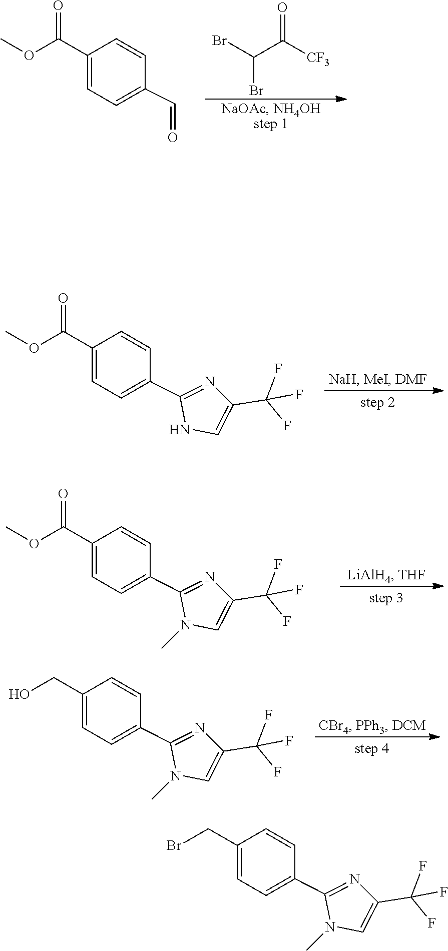

### Step 1. methyl 4-(4-(trifluoromethyl)-1H-imidazol-2-yl)benzoate

A mixture of 3,3-dibromo-1,1,1-trifluoropropan-2-one (36 g, 0.13 mol) and NaOAc (20 g, 0.24 mol) in water (80 mL) was heated at 95° C. for 30 min. After cooling to 0° C., a cold solution of methyl 4-formylbenzoate (20 g, 0.12 mol) in a mixture of ammonium hydroxide (28%, 100 mL) and MeOH (300 mL) was added. The resulting mixture was stirred at ambient temperature for 4 h then concentrated under vacuum and purified by silica gel chromatography (eluting with 1/2 EtOAc/PE) to afford methyl 4-(4-(trifluoromethyl)-1H-imidazol-2-yl)benzoate (30.1 g). MS (ESI) m/z 271.3 [M+H]⁺.

### Step 2. methyl 4-(1-methyl-4-(trifluoromethyl)-1H-imidazol-2-yl)benzoate

To a solution of methyl 4-(4-(trifluoromethyl)-1H-imidazol-2-yl)benzoate (10 g, 37 mmol) in DMF (100 mL) at 0° C. was added 60% NaH (2.2 g, 60% in mineral oil, 55 mmol). The mixture was stirred at 0° C. for 30 min then MeI (7.8 g, 55 mmol) was added. After stirring at ambient temperature for 2 h, the reaction mixture was diluted with water (300 mL) and extracted with EtOAc (3×300 mL). The combined organic phases were washed with brine, dried over anhydrous Na₂SO₄, concentrated under vacuum to afford methyl 4-(1-methyl-4-(trifluoromethyl)-1H-imidazol-2-yl)benzoate (9.8 g), which was used directly in next step without further purification. MS (ESI) m/z 285.3 [M+H]⁺.

### Step 3. (4-(1-methyl-4-(trifluoromethyl)-1H-imidazol-2-yl)phenyl)methanol

To a solution of methyl 4-(1-methyl-4-(trifluoromethyl)-1H-imidazol-2-yl)benzoate (5 g , 17.6 mmol) in dry THF (50 mL) at 0° C. was added LiAlH₄ (3.3 g, 88 mmol). The reaction mixture was stirred at room temperature for 30 min then quenched with saturated Na₂CO₃ (10 mL). The mixture was diluted with DCM (50 mL) and filtered through celite. The filtrate was concentrated and purified by silica gel chromatography (eluting with 1/10 MeOH/DCM) to afford (4-(1-methyl-4-(trifluoromethyl)-1H-imidazol-2-yl)phenyl)methanol (3.0 g). MS (ESI) m/z 257.5 [M+H]⁺.

### Step 4. 2-(4-(bromomethyl)phenyl)-1-methyl-4-(trifluoromethyl)-1H-imidazole

To a mixture of (4-(1-methyl-4-(trifluoromethyl)-1H-imidazol-2-yl)phenyl)methanol (5 g, 19.5 mmol), PPh₃ (10 g, 39.1 mmol) and NaHCO₃ (3.3 g, 39.1 mmol) in DCM (100 mL) was added CBr₄ (13 g, 39.1 mmol) slowly at room temperature. The resulting mixture was stirred at ambient temperature for 1 h then concentrated under vacuum and purified by silica gel chromatography (eluting with 1/5 EtOAc/PE) to afford 2-(4-(bromomethyl)phenyl)-1-methyl-4-(trifluoromethyl)-1H-imidazole (3.2 g). ¹H NMR (400 MHz, DMSO-d₆) δ 7.95 (s, 1H), 7.73 (d, J=8.1 Hz, 2H), 7.59 (d, J=8.1 Hz, 2H), 4.78 (s, 2H), 3.80 (s, 3H). MS (ESI) m/z 319.3, 321.3 [M+H]⁺.

### Intermediate BB-2. 2-(4-(bromomethyl)phenyl)-1-ethyl-4-(trifluoromethyl)-1H-imidazole

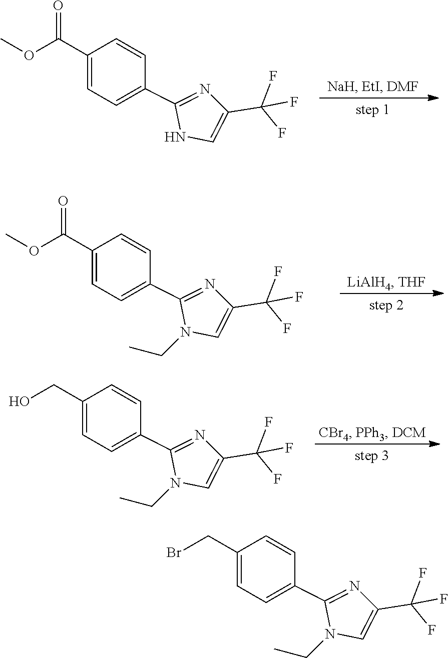

2-(4-(bromomethyl)phenyl)-1-ethyl-4-(trifluoromethyl)-1H-imidazole was prepared from methyl 4-(4-(trifluoromethyl)-1H-imidazol-2-yl)benzoate by following steps 2 to step 4 of Intermediate BB-1. ¹H NMR (400 MHz, Chloroform-d) δ 7.57 (d, J=8.3 Hz, 2H), 7.49 (d, J=8.3 Hz, 2H), 7.37 (d, J=1.1 Hz, 1H), 4.53 (s, 2H), 4.08 (q, J=7.3 Hz, 2H), 1.45 (t, J=7.3 Hz, 3H). MS (ESI) m/z 333.0, 335.0 [M+H]⁺.

### Intermediate BB-3. 2-(4-(bromomethyl)phenyl)-1-isopropyl-4-(trifluoromethyl)-1H-imidazole

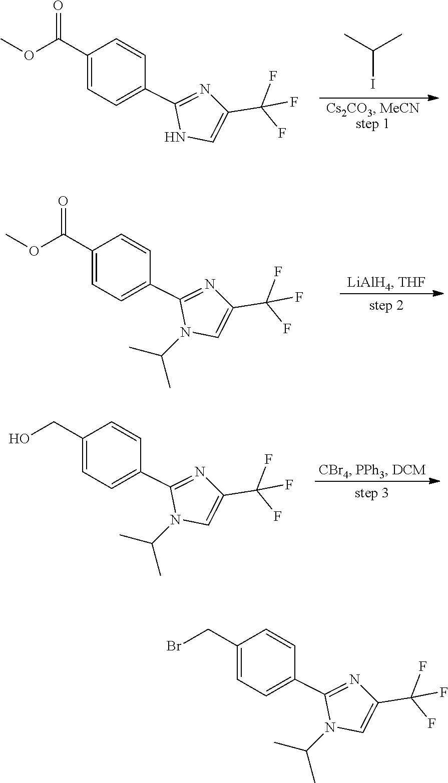

### Step 1. methyl 4-(1-isopropyl-4-(trifluoromethyl)-1H-imidazol-2-yl)benzoate

A mixture of methyl 4-(4-(trifluoromethyl)-1H-imidazol-2-yl)benzoate (10 g, 37 mmol), 2-iodopropane (9.4 g, 55.5 mmol) and Cs₂CO₃ (36.1 g, 111 mmol) in MeCN (100 mL) was stirred at ambient temperature overnight. The mixture concentrated under vacuum, diluted with water (100 mL) and extracted with EtOAc (3×100 mL). The combined organic layers were dried over anhydrous Na₂SO₄, concentrated under vacuum and purified by silica gel chromatography (eluting with 1/4 EtOAc/PE) to afford methyl 4-(1-isopropyl-4-(trifluoromethyl)-1H-imidazol-2-yl)benzoate (7.0 g). MS (ESI) m/z 313.1 [M+H]⁺.

### Step 2. (4-(1-isopropyl-4-(trifluoromethyl)-1H-imidazol-2-yl)phenyl)methanol

(4-(1-isopropyl-4-(trifluoromethyl)-1H-imidazol-2-yl)phenyl)methanol was synthesized from methyl 4-(1-isopropyl-4-(trifluoromethyl)-1H-imidazol-2-yl)benzoate according to step 3 of Intermediate BB-1. MS (ESI) m/z 285.3 [M+H]⁺.

### Step 3. 2-(4-(bromomethyl)phenyl)-1-isopropyl-4-(trifluoromethyl)-1H-imidazole

2-(4-(bromomethyl)phenyl)-1-isopropyl-4-(trifluoromethyl)-1H-imidazole was prepared from (4-(1-isopropyl-4-(trifluoromethyl)-1H-imidazol-2-yl)phenyl)methanol according to step 4 of Intermediate BB-1. ¹H NMR (400 MHz, Chloroform-d) δ 7.55-7.48 (m, 4H), 7.43 (d, J=1.1 Hz, 1H), 4.62-4.55 (m, 1H), 4.53 (s, 2H), 1.46 (d, J=6.7 Hz, 6H). MS (ESI) m/z 346.9, 348.9 [M+H]⁺.

### Intermediate BB-4. 2-(4-(bromomethyl)-2-fluorophenyl)-1-methyl-4-(trifluoromethyl)-1H-imidazole

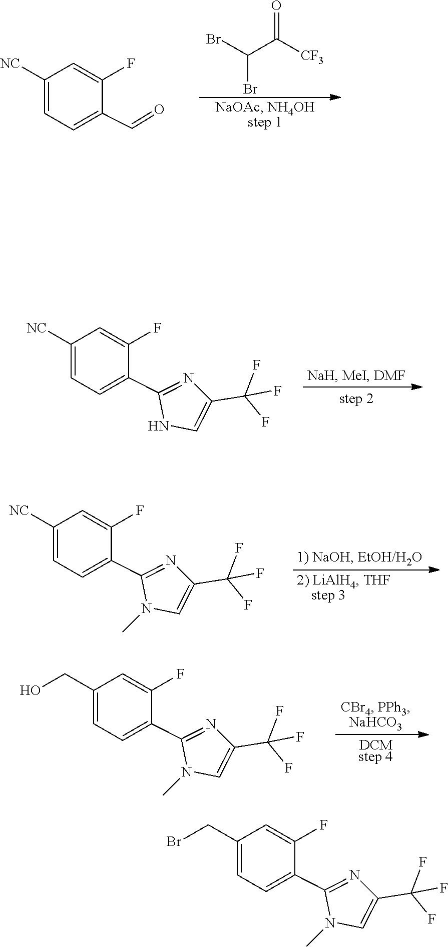

### Step 1. 3-fluoro-4-(4-(trifluoromethyl)-1H-imidazol-2-yl)benzonitrile

3-fluoro-4-(4-(trifluoromethyl)-1H-imidazol-2-yl)benzonitrile was prepared from 3-fluoro-4-formylbenzonitrile by following step 1 of Intermediate BB-1. MS (ESI) m/z 256.1 [M+H]⁺.

### Step 2. 3-fluoro-4-(1-methyl-4-(trifluoromethyl)-1H-imidazol-2-yl)benzonitrile

3-fluoro-4-(1-methyl-4-(trifluoromethyl)-1H-imidazol-2-yl)benzonitrile was synthesized from 3-fluoro-4-(4-(trifluoromethyl)-1H-imidazol-2-yl)benzonitrile according to step 2 of Intermediate BB-1. MS (ESI) m/z 270.1 [M+H]⁺.

### Step 3. (3-fluoro-4-(1-methyl-4-(trifluoromethyl)-1H-imidazol-2-yl)phenyl)methanol

A mixture of 3-fluoro-4-(1-methyl-4-(trifluoromethyl)-1H-imidazol-2-yl)benzonitrile (1.12 g, 4.11 mmol) and NaOH (3.32 g, 82.3 mmol) in EtOH/H₂O (50 mL/25 mL) was heated at 100° C. for 3 h. The mixture was concentrated under vacuum to remove the EtOH and the pH adjusted to 3 by slow addition of 3M HCl. The resulting solution was extracted with EtOAc (3×50 mL). The organic layers were combined, dried over Na₂SO₄, filtered and concentrated under vacuum. The residue was dissolved in dry THF (10 mL) and cooled to 0° C., LiAlH₄ (312 mg, 8.21 mmol) was added slowly and the reaction mixture allowed to warm to ambient temperature. The reaction was stirred for 20 min, then quenched with 2M NaOH and diluted with DCM (20 mL). The resulting mixture was filtered through celite and the filtrate dried over Na₂SO₄ and concentrated under vacuum to afford (3-fluoro-4-(1-methyl-4-(trifluoromethyl)-1H-imidazol yl)phenyl)methanol (1.01 g). MS (ESI) m/z 275.2 [M+H]⁺.

### Step 4. 2-(4-(bromomethyl)-2-fluorophenyl)-1-methyl-4-(trifluoromethyl)-1H-imidazole

2-(4-(bromomethyl)-2-fluorophenyl)-1-methyl-4-(trifluoromethyl)-1H-imidazole was prepared from (3-fluoro-4-(1-methyl-4-(trifluoromethyl)-1H-imidazol-2-yl)phenyl)methanol according to step 4 of Intermediate BB-1. ¹H NMR (400 MHz, Chloroform-d) δ 7.60 (t, J=7.6 Hz, 1H), 7.39-7.34 (m, 1H), 7.30 (dd, J=7.9, 1.6 Hz, 1H), 7.25-7.21 (m, 1H), 4.49 (s, 2H), 3.66 (d, J=2.4 Hz, 3H). MS (ESI) m/z 337.1, 339.2 [M+H]⁺.

### Intermediate BB-5. 2-(4-(bromomethyl)-2-fluorophenyl)-1-ethyl-4-(trifluoromethyl)-1H-imidazole

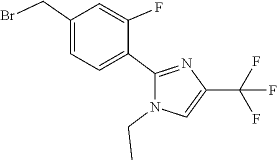 [SMILES: CCn1cc(C(F)(F)F)nc1-c1ccc(CBr)cc1F]

2-(4-(bromomethyl)-2-fluorophenyl)-1-ethyl-4-(trifluoromethyl)-1H-imidazole was prepared from 3-fluoro-4-(4-(trifluoromethyl)-1H-imidazol-2-yl)benzonitrile and iodoethane following step 2 to step 4 of Intermediate BB-4. ¹H NMR (400 MHz, Chloroform-d) δ 7.56 (t, J=7.6 Hz, 1H), 7.42 (s, 1H), 7.30 (dd, J=7.9, 1.4 Hz, 1H), 7.23 (d, J=10.4 Hz, 1H), 4.49 (s, 2H), 3.94 (q, J=7.3 Hz, 2H), 1.41 (t, J=7.3 Hz, 3H).

### Intermediate BB-6. 2-(4-(bromomethyl)-2-fluorophenyl)-1-isopropyl-4-(trifluoromethyl)-1H-imidazole

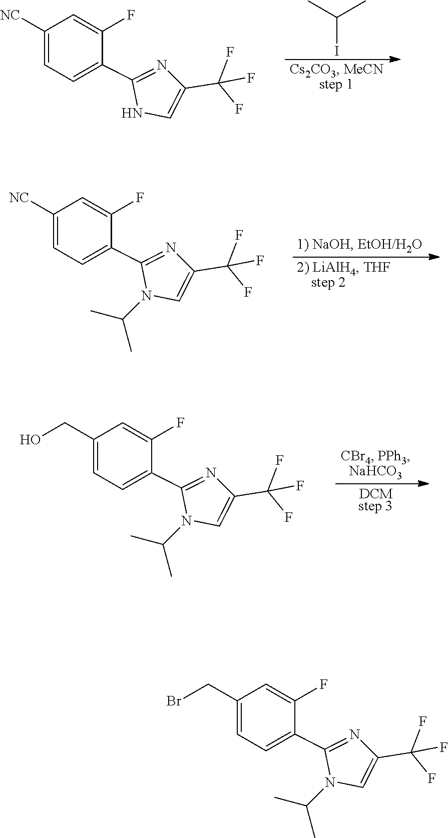

### Step 1. 3-fluoro-4-(1-isopropyl-4-(trifluoromethyl)-1H-imidazol-2-yl)benzonitrile

3-fluoro-4-(1-isopropyl-4-(trifluoromethyl)-1H-imidazol-2-yl)benzonitrile was prepared from 3-fluoro-4-(4-(trifluoromethyl)-1H-imidazol-2-yl)benzonitrile following step 1 of Intermediate BB-3. MS (ESI) m/z 298.1 [M+H]⁺.

### Step 2. (3-fluoro-4-(1-isopropyl-4-(trifluoromethyl)-1H-imidazol-2-yl)phenyl)methanol

(3-fluoro-4-(1-isopropyl-4-(trifluoromethyl)-1H-imidazol-2-yl)phenyl)methanol was prepared from 3-fluoro-4-(1-isopropyl-4-(trifluoromethyl)-1H-imidazol-2-yl)benzonitrile according to step 3 of Intermediate BB-4. MS (ESI) m/z 303.2 [M+H]⁺.

### Step 3. 2-(4-(bromomethyl)-2-fluorophenyl)-1-isopropyl-4-(trifluoromethyl)-1H-imidazole

2-(4-(bromomethyl)-2-fluorophenyl)-1-isopropyl-4-(trifluoromethyl)-1H-imidazole was prepared from (3-fluoro-4-(1-isopropyl-4-(trifluoromethyl)-1H-imidazol-2-yl)phenyl)methanol according to step 4 of Intermediate BB-1.¹H NMR (400 MHz, Chloroform-d) δ 7.53 (t, J=7.6 Hz, 1H), 7.45 (d, J=1.1 Hz, 1H), 7.29 (dd, J=7.9, 1.7 Hz, 1H), 7.23 (dd, J=10.3, 1.6 Hz, 1H), 4.49 (s, 2H), 4.36-4.19 (m, 1H), 1.43 (d, J=6.7 Hz, 6H).

### Intermediate BB-7. 1-(4-(bromomethyl)phenyl)-5-methyl-3-(trifluoromethyl)-1H-pyrazole

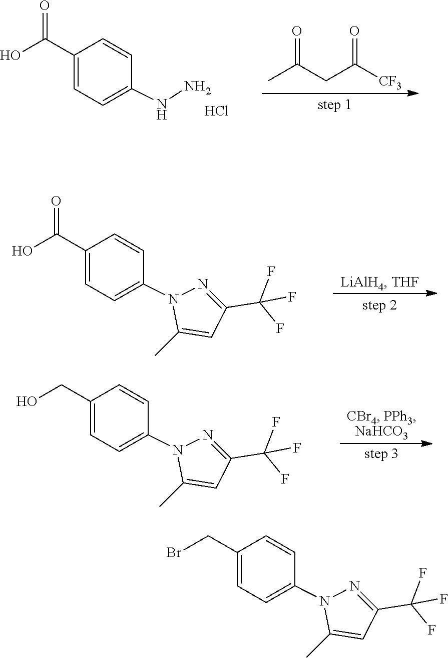

### Step 1. 4-(5-methyl-3-(trifluoromethyl)-1H-pyrazol-1-yl)benzoic acid

To a solution of 4-hydrazinylbenzoic acid hydrochloride (10 g, 53 mmol) in EtOH (100 mL) was slowly added 1,1,1-trifluoropentane-2,4-dione (8.2 g, 53 mmol). The solution was stirred at ambient temperature for 2 h then concentrated under vacuum and purified by silica gel chromatography (eluting with 1/10 MeOH/DCM) to afford 4-(5-methyl (trifluorometH-yl)-1H-pyrazol-1-yl)benzoic acid (4.2 g). MS (ESI) m/z 271.1 [M+H]⁺.

### Step 2. (4-(5-methyl-3-(trifluoromethyl)-1H-pyrazol-1-yl)phenyl)methanol

(4-(5-methyl-3-(trifluoromethyl)-1H-pyrazol-1-yl)phenyl)methanol was synthesized from 4-(5-methyl-3-(trifluoromethyl)-1H-pyrazol-1-yl)benzoic acid according to step 3 of Intermediate BB-1. MS (ESI) m/z 257.2 [M+H]⁺.

### Step 3. 1-(4-(bromomethyl)phenyl)-5-methyl-3-(trifluoromethyl)-1H-pyrazole

1-(4-(bromomethyl)phenyl)-5-methyl-3-(trifluoromethyl)-1H-pyrazole was prepared from (4-(5-methyl-3-(trifluoromethyl)-1H-pyrazol-1-yl)phenyl)methanol according to step 4 of Intermediate BB-1. MS (ESI) m/z 319.2, 341.22 [M+H]⁺.

### Intermediate BB-8. 1-(4-(bromomethyl)-2-fluorophenyl)-5-methyl-3-(trifluoromethyl)-1H-pyrazole

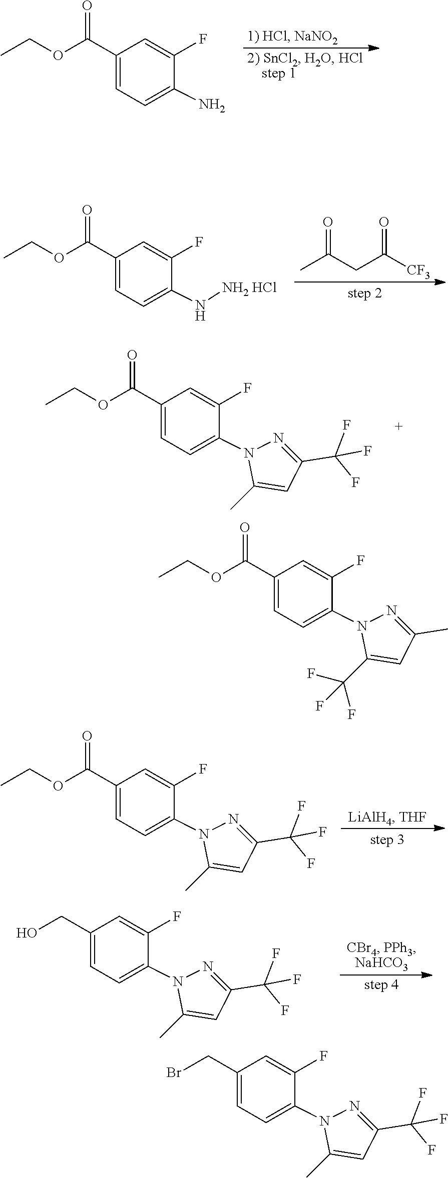

### Step 1. ethyl 3-fluoro-4-hydrazinylbenzoate hydrochloride

To a solution of ethyl 4-amino-3-fluorobenzoate (8.0 g, 43.7 mmol) in concentrated HCl (60 mL) was added a solution of NaNO₂ (3.59 g, 52.0 mmol) in H₂O (6 mL) at 0° C. After stirred at the same temperature for 30 min, the mixture was added dropwise to a solution of SnCl₂.2H₂O (32 g, 141 mmol) in concentrated HCl (80 mL) at −20° C. The reaction was stirred at −10° C. for 30 min then solid precipitate that formed was collected by filtration. The solid was dissolved in 10% Na₂CO₃ and extracted with EtOAc (3×100 mL). The organic layers were combined, dried over anhydrous sodium sulfate, filtered and concentrated under vacuum to afford ethyl 3-fluoro-4-hydrazinylbenzoate hydrochloride (7.2 g). MS (ESI) m/z 185.12 [M+H]⁺.

### Step 2. ethyl 3-fluoro-4-(5-methyl-3-(trifluoromethyl)-1H-pyrazol-1-yl)benzoate

To a solution of ethyl 3-fluoro-4-hydrazinylbenzoate hydrochloride (5 g, 21.3 mmol) in EtOH (50 mL) was slowly added 1,1,1-trifluoropentane-2,4-dione (4 g, 26 mmol). The solution was stirred at ambient temperature for 2 h then concentrated under vacuum and purified by silica gel chromatography (eluting with 1/50 Acetone/PE) to ethyl 3-fluoro-4-(5-methyl-3-(trifluoromethyl)-1H-pyrazol-1-yl)benzoate (2.2 g) and ethyl 3-fluoro-4-(3-methyl-5-(trifluoromethyl)-1H-pyrazol-1-yl)benzoate (1.0 g).

ethyl 3-fluoro-4-(5-methyl-3-(trifluoromethyl)-1H-pyrazol-1-yl)benzoate. ¹H NMR (400 MHz, Chloroform-d) δ 8.02-7.89 (m, 2H), 7.62-7.53 (m, 1H), 6.48 (s, 1H), 4.42 (q, J=7.1 Hz, 2H), 2.26 (d, J=1.7 Hz, 3H), 1.42 (t, J=7.1 Hz, 3H).

ethyl 3-fluoro-4-(3-methyl-5-(trifluoromethyl)-1H-pyrazol-1-yl)benzoate. ¹H NMR (400 MHz, Chloroform-d) δ 7.98-7.86 (m, 2H), 7.50 (t, J=7.7 Hz, 1H), 6.63 (s, 1H), 4.41 (q, J=7.1 Hz, 2H), 2.37 (s, 3H), 1.41 (t, J=7.1 Hz, 3H).

### Step 3. (3-fluoro-4-(5-methyl-3-(trifluoromethyl)-1H-pyrazol-1-yl)phenyl)methanol

(3-fluoro-4-(5-methyl-3-(trifluoromethyl)-1H-pyrazol-1-yl)phenyl)methanol was prepared from ethyl 3-fluoro-4-(5-methyl-3-(trifluoromethyl)-1H-pyrazol-1-yl)benzoate according to step 3 of Intermediate BB-1. MS (ESI) m/z 275.11 [M+H]⁺.

### Step 4. 1-(4-(bromomethyl)-2-fluorophenyl)-5-methyl-3-(trifluoromethyl)-1H-pyrazole

1-(4-(bromomethyl)-2-fluorophenyl)-5-methyl-3-(trifluoromethyl)-1H-pyrazole was synthesized from (3-fluoro-4-(5-methyl-3-(trifluoromethyl)-1H-pyrazol-1-yl)phenyl)methanol according to step 4 of Intermediate BB-1. ¹H NMR (400 MHz, Chloroform-d) δ 7.48-7.42 (m, 1H), 7.32 (d, J=2.1 Hz, 1H), 7.31-7.27 (m, 1H), 6.46 (s, 1H), 4.49 (s, 2H), 2.26 (s, 3H).

### Intermediate BB-9. 1-(4-(bromomethyl)phenyl)-5-methoxy-3-(trifluoromethyl)-1H-pyrazole

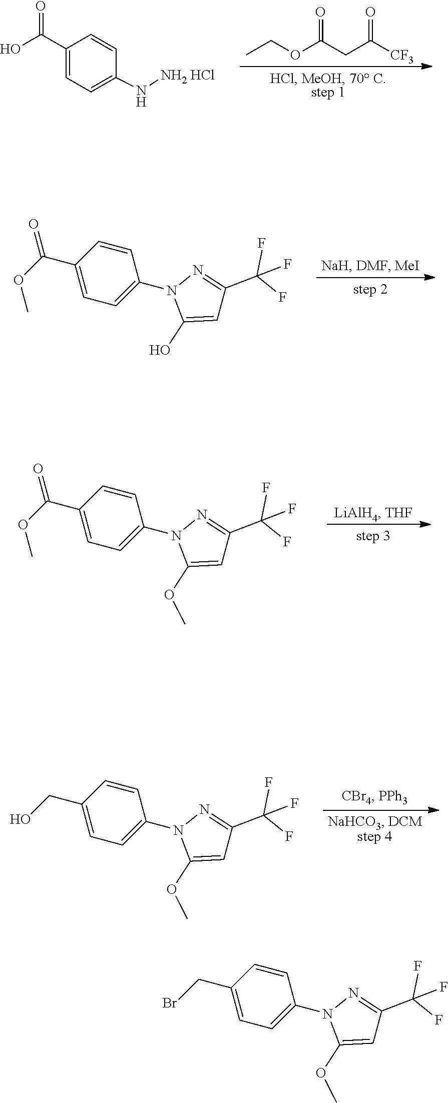

### Step 1. methyl 4-(5-hydroxy-3-(trifluoromethyl)-1H-pyrazol-1-yl)benzoate

A solution of 4-hydrazinylbenzoic acid hydrochloride (5.0 g, 26.5 mmol) and ethyl 4,4,4-trifluoro-3-oxobutanoate (4.9 g, 26.51 mmol) in a mixture of MeOH (60 mL) and concentrated HCl (12 mL) was heated at 70° C. for 12 h. After cooling to ambient temperature, solvent was removed under reduced pressure to give methyl 4-(5-hydroxy-3-(trifluoromethyl)-1H-pyrazol-1-yl) benzoate (7.5 g). The crude product was used in next step directly without further purification. MS (ESI) m/z 287.1 [M+H]⁺.

### Step 2. methyl 4-(5-methoxy-3-(trifluoromethyl)-1H-pyrazol-1-yl)benzoate

To a solution of methyl 4-(5-hydroxy-3-(trifluoromethyl)-1H-pyrazol-1-yl)benzoate (1.1 g 3.85 mmol) in DMF at 0° C. (5 mL) was added NaH (230.8 mg, 60% in mineral oil, 5.77 mmol). the reaction was stirred at 0° C. for 30 min then methyl iodide (546.7 mg, 3.85 mmol) was added. The reaction was allowed to warm to ambient temperature and stirred 1 h then quenched with ice water, extracted with EtOAc (200 mL) and concentrated to give a crude product. The crude was purified on a Biotage Isolera One (C₁₈ column, eluting with 10% to 90% MeCN/H₂O) to afford methyl 4-(5-methoxy-3-(trifluoromethyl)-1H-pyrazol-1-yl)benzoate (715 mg). MS (ESI) m/z 301.1 [M+H]⁺.

### Step 3. (4-(5-methoxy-3-(trifluoromethyl)-1H-pyrazol-1-yl)phenyl)methanol

(4-(5-methoxy-3-(trifluoromethyl)-1H-pyrazol-1-yl)phenyl)methanol was synthesized from methyl 4-(5-methoxy-3-(trifluoromethyl)-1H-pyrazol-1-yl)benzoate according to step 3 of Intermediate BB-1. MS (ESI) m/z 273.1 [M+H]⁺.

### Step 4. 1-(4-(bromomethyl)phenyl)-5-methoxy-3-(trifluoromethyl)-1H-pyrazole

1-(4-(bromomethyl)phenyl)-5-methoxy-3-(trifluoromethyl)-1H-pyrazole was synthesized from (4-(5-methoxy-3-(trifluoromethyl)-1H-pyrazol-1-yl)phenyl)methanol according to step 4 of Intermediate BB-1. ¹H NMR (400 MHz, Chloroform-d) δ 7.72-7.66 (m, 2H), 7.51-7.42 (m, 2H), 5.95 (s, 1H), 4.51 (s, 2H), 3.99 (s, 3H). MS (ESI) m/z 333.0, 335.0 [M+H]⁺.

### Intermediate BB-10. 1-(4-(bromomethyl)-2-fluorophenyl)-5-methoxy-3-(trifluoromethyl)-1H-pyrazole

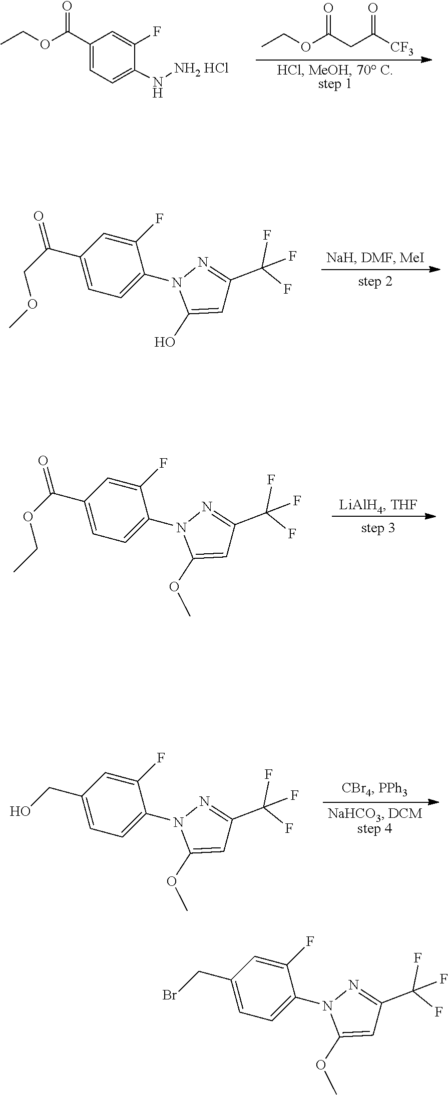

1-(4-(bromomethyl)-2-fluorophenyl)-5-methoxy-3-(trifluoromethyl)-1H-pyrazole was prepared from ethyl 3-fluoro-4-hydrazinylbenzoate hydrochloride (prepared in step 1 of Intermediate BB-8) according to Intermediate BB-9. ¹H NMR (400 MHz, Chloroform-d) δ 7.48-7.39 (m, 1H), 7.27-7.22 (m, 2H), 5.91 (s, 1H), 4.45 (s, 2H), 3.93 (s, 3H). MS (ESI) m/z 351.0, 353.0 [M+H]⁺.

### Intermediate BB-11. 3-(4-(bromomethyl)phenyl)-1-methyl-5-(trifluoromethyl)-1H-1,2,4-triazole

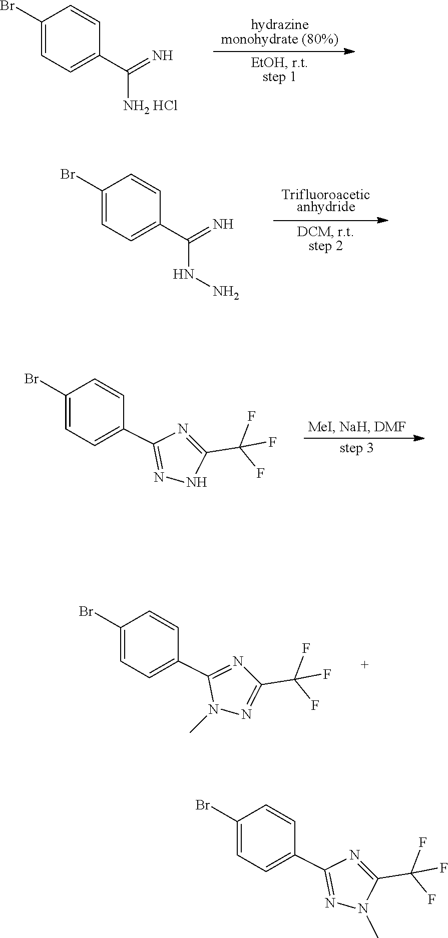

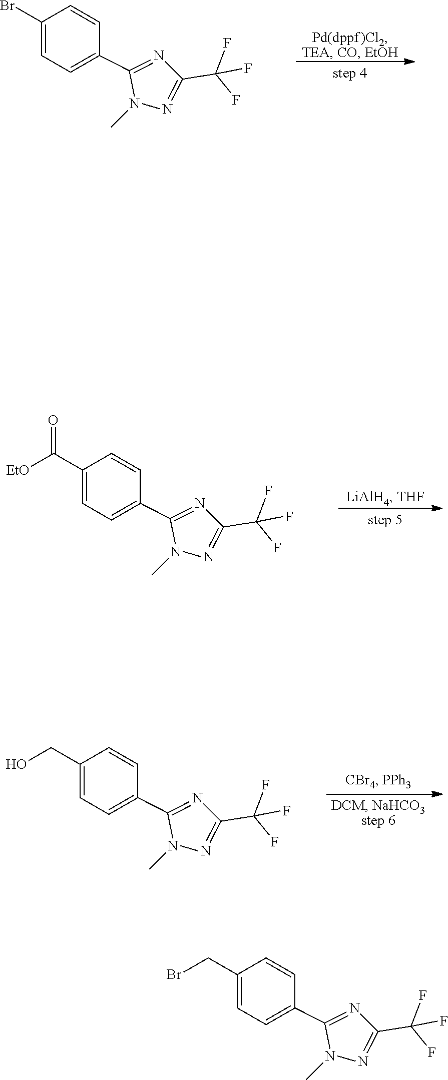

### Step 1: 4-bromobenzimidohydrazide

A mixture of 4-bromobenzimidamide hydrochloride (26.2 g, 111.3 mmol), 80% hydrazine monohydrate (30 mL) in EtOH (150 mL) was stirred at room temperature for 3 h. The reaction mixture was diluted with H₂O (200 mL) and extracted with EtOAc (3×150 mL). The organic layers were combined, dried over anhydrous Na₂SO₄, filtered, concentrated under vacuum to give 4-bromobenzimidohydrazide (21.9 g), which was used directly in next step without further purification. MS (ESI) m/z 214.0, 216.0 [M+H]⁺.

### Step 2: 3-(4-bromophenyl)-5-(trifluoromethyl)-1H-1,2,4-triazole

A mixture of 4-bromobenzimidohydrazide (21.9 g, 102.2 mmol), trifluoroacetic anhydride (32.2 g, 153.3 mmol) in DCM (150 mL) was stirred at room temperature for 3 h. The mixture was concentrated under vacuum and purified by column chromatography (eluting with 10% to 50% EtOAc/PE) to give 3-(4-bromophenyl)-5-(trifluoromethyl)-1H-1,2,4-triazole (17 g,). MS (ESI) m/z 292.0, 294.0 [M+H]⁺.

### Step 3: 5-(4-bromophenyl)-1-methyl-3-(trifluoromethyl)-1H-1,2,4-triazole and 3-(4-bromophenyl)-1-methyl-5-(trifluoromethyl)-1H-1,2,4-triazole

A mixture of 3-(4-bromophenyl)-5-(trifluoromethyl)-1H-1,2,4-triazole (13.5 g, 46.4 mmol) in DMF (50 mL) at 0° C. was treated with portion-wise with NaH (2.78 g, 60% in mineral oil, 69.6 mmol). The resulting mixture was stirred at room temperature for 30 min. lodomethane (13.2 g, 92.8 mmol) was added and the resulting solution stirred for 1 h at room temperature. The reaction mixture was poured into ice water (50 mL), extracted with EtOAc (3×150 mL). The organic layers were combined, dried over anhydrous Na₂SO₄, filtered, concentrated and purified by column chromatography (eluting with 0 to 33.3% EtOAc/PE) to give 5-(4-bromophenyl)-1-methyl-3-(trifluoromethyl)-1H-1,2,4-triazole (4.3 g) and 3-(4-bromophenyl)-1-methyl-5-(trifluoromethyl)-1H-1,2,4-triazole (4.7 g). MS (ESI) m/z 306.0, 307.9 [M+H]⁺.

### Step 4: ethyl 4-(1-methyl-3-(trifluoromethyl)-1H-1,2,4-triazol-5-yl)benzoate

A mixture of 5-(4-bromophenyl)-1-methyl-3-(trifluoromethyl)-1H-1,2,4-triazole (4.3 g, 14.1 mmol), Pd(dppf)Cl₂ (1.54 g, 2.11 mmol) and triethylamine (7.12 g, 70.5 mmol) in EtOH (30 mL) was heated at 110° C. under an atmosphere of carbon monoxide (450 psi) for 12 h. The mixture was cooled to room temperature, filtered through celite, concentrated and purified by column chromatography (eluting with 0 to 20% EtOAc/PE) to give ethyl 4-(1-methyl-3-(trifluoromethyl)-1H-1,2,4-triazol-5-yl)benzoate (3.47 g). MS (ESI) m/z 300.15 [M+H]⁺.

### Step 5. (4-(1-methyl-3-(trifluoromethyl)-1H-1,2,4-triazol-5-yl)phenyl)methanol

(4-(1-methyl-3-(trifluoromethyl)-1H-1,2,4-triazol-5-yl)phenyl)methanol was synthesized from ethyl 4-(1-methyl-3-(trifluoromethyl)-1H-1,2,4-triazol-5-yl)benzoate according to step 3 of Intermediate BB-1. MS (ESI) m/z 258.2 [M+H]⁺.

### Step 6. 5-(4-(bromomethyl)phenyl)-1-methyl-3-(trifluoromethyl)-1H-1,2,4-triazole

5-(4-(bromomethyl)phenyl)-1-methyl-3-(trifluoromethyl)-1H-1,2,4-triazole was prepared from (4-(1-methyl-3-(trifluoromethyl)-1H-1,2,4-triazol-5-yl)phenyl)methanol according to step 4 of Intermediate BB-1. ¹H NMR (400 MHz, DMSO-d₆) δ 7.85 (d, J=8.4 Hz, 2H), 7.68 (d, J=8.4 Hz, 2H), 4.80 (s, 2H), 4.07 (s, 3H). MS (ESI) m/z 320.0, 322.0 [M+H]⁺.

### Intermediate BB-12. 3-(4-(bromomethyl)phenyl)-1-methyl-5-(trifluoromethyl)-1H-1,2,4-triazole

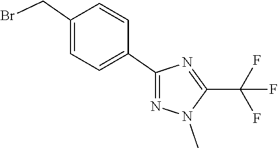 [SMILES: Cn1nc(-c2ccc(CBr)cc2)nc1C(F)(F)F]

3-(4-(bromomethyl)phenyl)-1-methyl-5-(trifluoromethyl)-1H-1,2,4-triazole was prepared from 3-(4-bromophenyl)-1-methyl-5-(trifluoromethyl)-1H-1,2,4-triazole (prepared in step 3 of Intermediate BB-11) following step 4 to step 6 of Intermediate BB-11. ¹H NMR (400 MHz, DMSO-d₆) δ 7.99 (d, J=8.3 Hz, 2H), 7.57 (d, J=8.4 Hz, 2H), 4.76 (s, 2H), 4.10 (d, J=1.1 Hz, 3H).

### Intermediate BB-13. 1-(4-(bromomethyl)phenyl)-5-methoxy-1H-pyrazole-3-carbonitrile

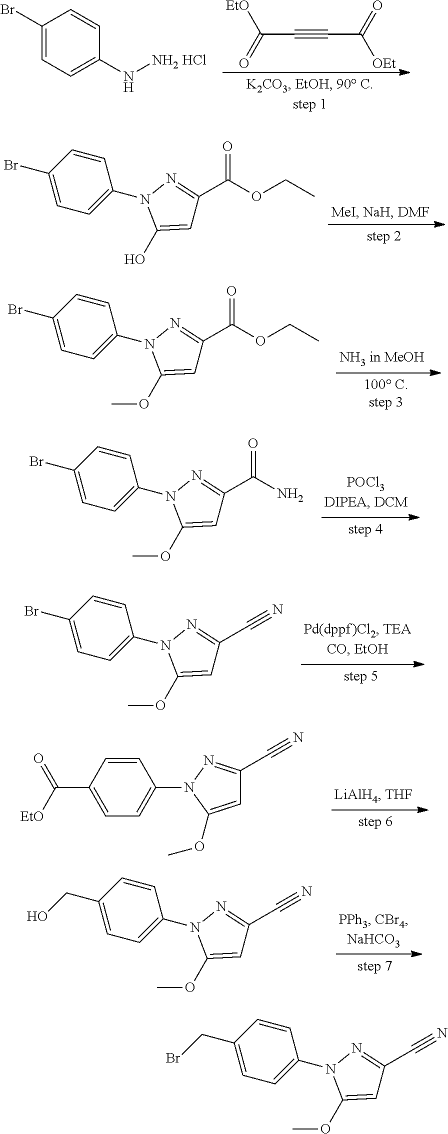

### Step 1. ethyl 1-(4-bromophenyl)-5-hydroxy-1H-pyrazole-3-carboxylate

To a solution of (4-bromophenyl)hydrazine hydrochloride (35.0 g, 156.6 mmol) in EtOH (500 mL) was added diethyl but-2-ynedioate (26.6 g, 156.6 mmol) and K₂CO₃ (21.6 g, 156.6 mmol). The resulting solution was heated at 90° C. for 12 h. The reaction mixture was filtered through celite, concentrated and purified by silica gel chromatography (eluting with 1/2 EtOAc/PE) to afford ethyl 1-(4-bromophenyl)-5-hydroxy-1H-pyrazole-3-carboxylate (18.9 g). MS (ESI) m/z 311.0, 312.0 [M+H]⁺.

### Step 2. ethyl 1-(4-bromophenyl)-5-methoxy-1H-pyrazole-3-carboxylate

To a solution of ethyl 1-(4-bromophenyl)-5-hydroxy-1H-pyrazole-3-carboxylate (18.9 g, 60.8 mmol) in DMF (150 mL) at 0° C. was added NaH (3.45 g, 60% in mineral oil, 86.2 mmol). The resulting solution was stirred for at 0° C. for 30 min before MeI (12.2 g, 86.2 mmol) was added. The resulting solution was stirred at room temperature for 12 h then quenched with H₂O and extracted with EtOAc (2×200 mL). The organic phase was dried over Na₂SO₄, filtered, concentrated and purified by silica gel chromatography (eluting with 1/5 EtOAc/PE) to afford ethyl 1-(4-bromophenyl)-5-methoxy-1H-pyrazole-3-carboxylate (7.1 g). MS (ESI) m/z 325.0, 227.0 [M+H]⁺.

### Step 3. 1-(4-bromophenyl)-5-methoxy-1H-pyrazole-3-carboxamide

A mixture of ethyl 1-(4-bromophenyl)-5-methoxy-1H-pyrazole-3-carboxylate (7.1 g, 21.8 mmol) in 7 M NH₃ in MeOH (70 mL) was heated at 100° C. in a sealed tube for 12 h. The solvent was removed under vacuum to afford of 1-(4-bromophenyl)-5-methoxy-1H-pyrazole-3-carboxamide (6.4 g). MS (ESI) m/z 296.0, 297.1 [M+H]⁺.

### Step 4. 1-(4-bromophenyl)-5-methoxy-1H-pyrazole-3-carbonitrile

A mixture of 1-(4-bromophenyl)-5-methoxy-1H-pyrazole-3-carboxamide (6.4 g, 21.6 mmol), POCl₃ (9.35 g, 61.14 mmol) and DIPEA (7.89 g, 61.14 mmol) in DCM (90 mL) was stirred at room temperature for 12 h. The reaction mixture was quenched with H₂O and extracted with EtOAc (2×200 mL). The organic phase was dried over Na₂SO₄, filtered, concentrated and purified by silica gel chromatography (eluting with 1/5 EtOAc/PE) to afford 1-(4-bromophenyl)-5-methoxy-1H-pyrazole-3-carbonitrile (2.7 g). MS (ESI) m/z 278.0, 280.0 [M+H]⁺.

### Step 5. ethyl 4-(3-cyano-5-methoxy-1H-pyrazol-1-yl)benzoate

ethyl 4-(3-cyano-5-methoxy-1H-pyrazol-1-yl)benzoate was prepared from 1-(4-bromophenyl)-5-methoxy-1H-pyrazole-3-carbonitrile according to step 4 of Intermediate BB-11. MS (ESI) m/z 272.1 [M+H]⁺.

### Step 6. 1-(4-(hydroxymethyl)phenyl)-5-methoxy-1H-pyrazole-3-carbonitrile

1-(4-(hydroxymethyl)phenyl)-5-methoxy-1H-pyrazole-3-carbonitrile was prepared from ethyl 4-(3-cyano-5-methoxy-1H-pyrazol-1-yl)benzoate according to step 3 of Intermediate BB-1. MS (ESI) m/z 230.1 [M+H]+.

### Step 7. 1-(4-(bromomethyl)phenyl)-5-methoxy-1H-pyrazole-3-carbonitrile

1-(4-(bromomethyl)phenyl)-5-methoxy-1H-pyrazole-3-carbonitrile was prepared from 1-(4-(hydroxymethyl)phenyl)-5-methoxy-1H-pyrazole-3-carbonitrile according to step 4 of Intermediate BB-1. MS (ESI) m/z 292.1, 294.1 [M+H]⁺.

### Intermediate BB-14. 4-(1-ethyl-4-(trifluoromethyl)-1H-imidazol-2-yl)aniline

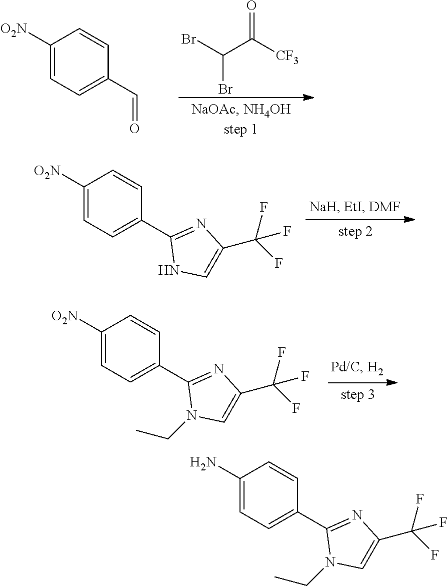

### Step 1 and step 2. 1-ethyl-2-(4-nitrophenyl)-4-(trifluoromethyl)-1H-imidazole

1-ethyl-2-(4-nitrophenyl)-4-(trifluoromethyl)-1H-imidazole was prepared from 4-nitrobenzaldehyde following step 1 and step 2 of intermediate BB-1. MS (ESI) m/z 286.1 [M+H]⁺.

### Step 3. 4-(1-ethyl-4-(trifluoromethyl)-1H-imidazol-2-yl)aniline

To a solution of 4-(1-ethyl-4-(trifluoromethyl)-1H-imidazol-2-yl)aniline (2.85 g, 10 mmol) in MeOH was added Pd/C (10%, 285 mg). The mixture was evacuated and backfilled with hydrogen three times and then charged with hydrogen. The resulting mixture was stirred for 16 h at ambient temperature, then was filtered through celite and concentrated under vacuum to give 4-(1-ethyl-4-(trifluoromethyl)-1H-imidazol-2-yl)aniline (2.35 g). MS (ESI) m/z 256.1 [M+H]⁺.

### Intermediate BB-15. 4-(1-ethyl-4-(trifluoromethyl)-1H-imidazol-2-yl)-3-fluoroaniline

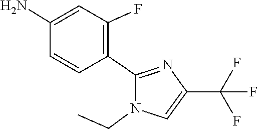 [SMILES: CCn1cc(C(F)(F)F)nc1-c1ccc(N)cc1F]

4-(1-ethyl-4-(trifluoromethyl)-1H-imidazol-2-yl)-3-fluoroaniline was prepared from 2-fluoro-4-nitrobenzaldehyde following Intermediate BB-14. MS (ESI) m/z 274.1 [M+H]⁺.

### Intermediate BB-16. 3-(4-(bromomethyl)phenyl)-1-methyl-1H-pyrazole

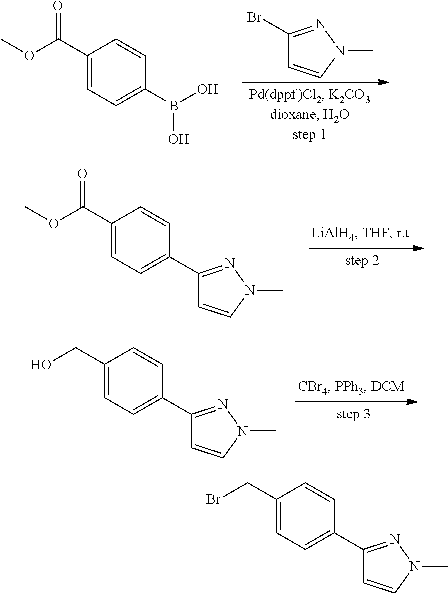

### Step 1. methyl 4-(1-methyl-1H-pyrazol-3-yl)benzoate

To a solution of 3-bromo-1-methyl-1H-pyrazole (4.0 g, 24.8 mmol) in mixture of dioxane (24 mL) and H₂O (6 mL) was added (4-(methoxycarbonyl)phenyl)boronic acid (8.9 g, 49.6 mmol), Pd(dppf)Cl₂ (906 mg, 1.24 mmol) and K₂CO₃ (7.0 g, 49.6 mmol). The mixture was heated at reflux for 16 h under N₂. After cooling to ambient temperature, the mixture was filtered through celite and the filtrate concentrated under vacuum. The residue was purified by silica gel chromatography (eluting with 1/10 EtOAc/PE) to afford methyl 4-(1,4-dimethyl-1H-pyrazol-3-yl)benzoate (3.2 g). MS (ESI) m/z 217.1 [M+H]⁺.

### Step 2. (4-(1-methyl-1H-pyrazol-3-yl)phenyl)methanol

(4-(1-methyl-1H-pyrazol-3-yl)phenyl)methanol was prepared from methyl 4-(1-methyl-1H-pyrazol-3-yl)benzoate according to step 3 of Intermediate BB-1. MS (ESI) m/z 189.1 [M+H]⁺.

### Step 3. 3-(4-(bromomethyl)phenyl)-1-methyl-1H-pyrazole

3-(4-(bromomethyl)phenyl)-1-methyl-1H-pyrazole was prepared from (4-(1-methyl-1H-pyrazol-3-yl)phenyl)methanol according to step 4 of Intermediate BB-1. MS (ESI) m/z 251.0, 253.0 [M+H]⁺.

### Intermediate BB-17. 3-(4-(bromomethyl)phenyl)-1,4-dimethyl-1H-pyrazole

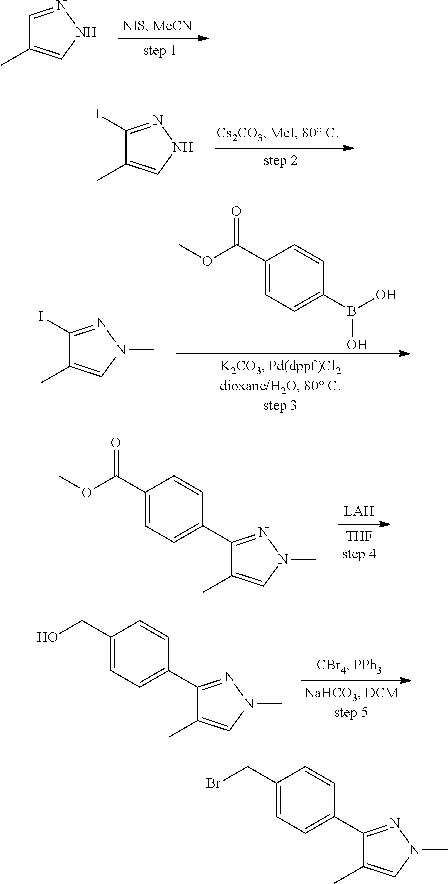

### Step 1. 3-iodo-4-methyl-1H-pyrazole

A mixture of 4-methyl-1H-pyrazole (10 g, 0.122 mol) and N-iodosuccinimide (30.18 g, 0.134 mol) in MeCN (50 mL) was stirred at 30° C. for 12 h. Solvent was removed and the crude was purified on a Biotage Isolera One (C₁₈ column, eluting with 10% to 90% MeCN/H₂O with 0.1% formic acid) to give 3-iodo-4-methyl-1H-pyrazole (3.8 g). ¹H NMR (400 MHz, Chloroform-d) δ 7.04 (s, 1H), 3.85 (s, 3H), 1.96 (s, 3H). MS (ESI) m/z 209.0 [M+H]⁺.

### Step 2. 3-iodo-1,4-dimethyl-1H-pyrazole

A mixture of 3-iodo-4-methyl-1H-pyrazole (4.8 g, 18.5 mmol), MeI (2.9 g, 20.3 mmol) and Cs₂CO₃ (12 g, 36.9 mmol) in MeCN (30 mL) was heated at 80° C. for 4 h. The reaction mixture was filtered through celite, concentrated under vacuum, diluted with water (100 mL) and extracted with EtOAc (3×100 mL). The combined organic layers were dried over anhydrous Na₂SO₄, concentrated under vacuum and purified by prep-HPLC (Conditions 3) to give 3-iodo-1,4-dimethyl-1H-pyrazole (1.4 g). ¹H NMR (400 MHz, Chloroform-d) δ 7.04 (s, 1H), 3.85 (s, 3H), 1.96 (s, 3H).

### Step 3. methyl 4-(1,4-dimethyl-1H-pyrazol-3-yl)benzoate

To a solution of 3-iodo-1,4-dimethyl-1H-pyrazole (1.4 g, 6.3 mmol) in a mixture of dioxane (8 mL) and H₂O (2 mL) was added (4-(methoxycarbonyl)phenyl)boronic acid (2.3 g, 13 mmol), Pd(dppf)Cl₂ (230 mg, 0.3 mmol) and K₂CO₃ (1.73 g, 12.6 mmol). The mixture was heated at 80° C. for 12 h under N₂. After cooling to ambient temperature, the mixture was filtered through celite and the filtrate concentrated under vacuum. The residue was purified by silica gel chromatography (eluting with 1/10 EtOAc/PE) to afford methyl 4-(1,4-dimethyl-1H-pyrazol-3-yl)benzoate (1.2 g). MS (ESI) m/z 231.1 [M+H]⁺.

### Step 4. (4-(1,4-dimethyl-1H-pyrazol-3-yl)phenyl)methanol

(4-(1,4-dimethyl-1H-pyrazol-3-yl)phenyl)methanol was prepared from methyl 4-(1,4-dimethyl-1H-pyrazol-3-yl)benzoate according to step 3 of Intermediate BB-1. MS (ESI) m/z 203.1 [M+H]+.

### Step 5. 3-(4-(bromomethyl)phenyl)-1,4-dimethyl-1H-pyrazole

3-(4-(bromomethyl)phenyl)-1,4-dimethyl-1H-pyrazole was prepared from (4-(1,4-dimethyl-1H-pyrazol-3-yl)phenyl)methanol according to step 4 of Intermediate BB-1. MS (ESI) m/z 265.1, 267.1 [M+H]⁺.

### Intermediate BB-18. 1-(4-(bromomethyl)phenyl)-5-(methoxymethyl)-3-(trifluoromethyl)-1H-pyrazole

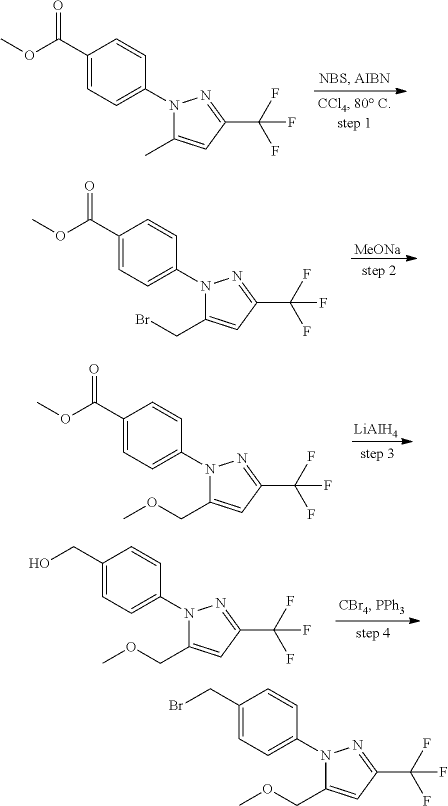

### Step 1. methyl 4-(5-(bromomethyl)-3-(trifluoromethyl)-1H-pyrazol-1-yl)benzoate

A mixture of methyl 4-(5-methyl-3-(trifluoromethyl)-1H-pyrazol-1-yl)benzoate (1.4 g, 4.9 mmol), NBS (0.8 g, 4.3 mmol) and AIBN (80.9 mg,0.49 mmol) in CCl₄(30 mL) was stirred at 80° C. overnight. The reaction mixture was diluted with water (30 mL) and extracted with DCM (20 mL×3). The combined organic layer was concentrated and purified by prep-HPLC (Conditions 2) to get methyl 4-(5-(bromomethyl)-3-(trifluoromethyl)-1H-pyrazol-1-yl)benzoate (570 mg). MS (ESI) m/z 363.0, 365.0 [M+H]⁺.

### Step 2. methyl 4-(5-(methoxymethyl)-3-(trifluoromethyl)-1H-pyrazol-1-yl)benzoate

A mixture of methyl 4-(5-(bromomethyl)-3-(trifluoromethyl)-1H-pyrazol-1-yl)benzoate (570 mg, 1.5 mmol) and MeONa (170.1 mg, 3.1 mmol) in MeOH (8.5 mL) was stirred at 90° C. for 0.5 h. The reaction mixture was diluted with water (20 mL), extracted with DCM (20 mL×3), the combined organic layer was dried over Na₂SO₄, concentrated under vacuum to give crude product (500 mg), which was used directly in next step without further purification. MS (ESI) m/z 315.1 [M+H]⁺.

### Step 3. (4-(5-(methoxymethyl)-3-(trifluoromethyl)-1H-pyrazol-1-yl)phenyl)methanol

(4-(5-(methoxymethyl)-3-(trifluoromethyl)-1H-pyrazol-1-yl)phenyl)methanol was prepared from methyl 4-(5-(methoxymethyl)-3-(trifluoromethyl)-1H-pyrazol yl)benzoate according to step 3 of Intermediate BB-1. MS (ESI) m/z 287.1 [M+H]⁺.

### Step 4. 1-(4-(bromomethyl)phenyl)-5-(methoxymethyl)-3-(trifluoromethyl)-1H-pyrazole

1-(4-(bromomethyl)phenyl)-5-(methoxymethyl)-3-(trifluoromethyl)-1H-pyrazole was prepared from (4-(5-(methoxymethyl)-3-(trifluoromethyl)-1H-pyrazol-1-yl)phenyl)methanol according to step 4 of Intermediate BB-1. MS (ESI) m/z 349.0, 351.0 [M+H]⁺.

### Intermediate BB-19. tert-butyl ((1-(4-(bromomethyl)phenyl)-3-(trifluoromethyl)-1H-pyrazol-5-yl) methyl)(methyl)carbamate

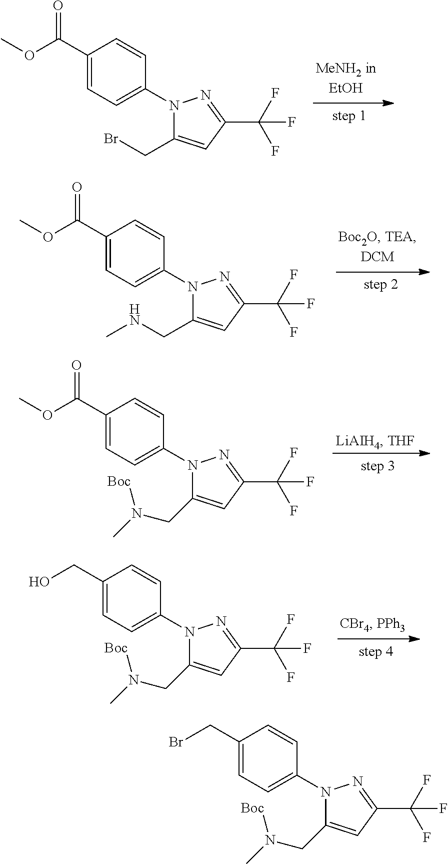

### Step 1. methyl 4-(5-((methylamino)methyl)-3-(trifluoromethyl)-1H-pyrazol-1-yl)benzoate

A solution of methyl 4-(5-(bromomethyl)-3-(trifluoromethyl)-1H-pyrazol-1-yl)benzoate (1g, 2.75 mmol) in ethanolic methylamine solution (2M, 10 mL) was stirred at room temperature overnight. Solvent was removed and the crude was purified by silica gel chromatography (eluting with 1/4 EtOAc/PE) to afford methyl 4-(5-((methylamino)methyl)-3-(trifluoromethyl)-1H-pyrazol-1-yl)benzoate (0.6 g). MS (ESI) m/z 314.1 [M+H]⁺.

### Step 2. methyl 4-(5-(((tert-butoxycarbonyl)(methyl)amino)methyl)-3-(trifluoromethyl)-1H-pyrazol-1-yl)benzoate

To a solution of methyl 4-(5-((methylamino)methyl)-3-(trifluoromethyl)-1H-pyrazol-1-yl)benzoate (550 mg, 1.76 mmol) and di-tert-butyl dicarbonate (574.5 mg, 2.63 mmol) in DCM (10 mL) was added TEA (532.8 mg, 5.26 mmol). The resulting solution was stirred at room temperature for 2 h. Solvent was removed and the crude was purified by silica gel chromatography (eluting with 1/8 EtOAc/PE) to afford methyl 4-(5-(((tert-butoxycarbonyl)(methyl)amino)methyl)-3-(trifluoromethyl)-1H-pyrazol-1-yl)benzoate (450 mg). MS (ESI) m/z 414.2 [M+H]⁺.

### Step 3 and step 4. tert-butyl ((1-(4-(bromomethyl)phenyl)-3-(trifluoromethyl)-1H-pyrazol-5-yl) methyl)(methyl)carbamate

tert-butyl ((1-(4-(bromomethyl)phenyl)-3-(trifluoromethyl)-1H-pyrazol-5-yl) methyl)(methyl)carbamate was prepared from methyl 4-(5-(((tert-butoxycarbonyl)(methyl)amino)methyl)-3-(trifluoromethyl)-1H-pyrazol-1-yl) benzoate following step 3 and step 4 of intermediate BB-1. MS (ESI) m/z 448.1, 450.1 [M+H]⁺.

### Intermediate BB-20. 5-bromo-2-(4-(1-bromoethyl)phenyl)-1-methyl-4-(trifluoromethyl)-1H-imidazole

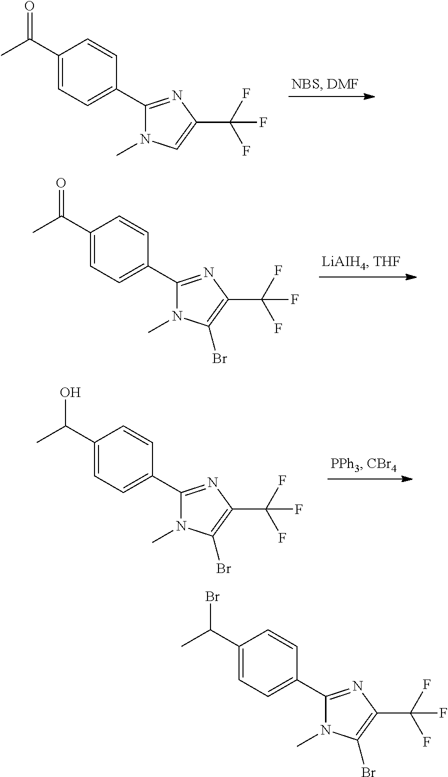

### Step 1. 1-(4-(5-bromo-1-methyl-4-(trifluoromethyl)-1H-imidazol-2-yl)phenyl)ethan-1-one

A solution of 1-(4-(1-methyl-4-(trifluoromethyl)-1H-imidazol-2-yl)phenyl)ethan-1-one (345 mg, 1.28 mmol) and NBS (217 mg, 1.22 mmol) in DMF (4 mL) was stirred at heated at 40° C. for 5 h. Solvent was removed and the crude was purified by silica gel chromatography (eluting with 1/5 EtOAc/PE) to afford 1-(4-(5-bromo-1-methyl (trifluoromethyl)-1H-imidazol-2-yl)phenyl)ethan-1-one (218 g). MS (ESI) m/z 347.0, 349.0 [M+H]⁺.

### Step 2 and step 3. 5-bromo-2-(4-(1-bromoethyl)phenyl)-1-methyl-4-(trifluoromethyl)-1H-imidazole

5-bromo-2-(4-(1-bromoethyl)phenyl)-1-methyl-4-(trifluoromethyl)-1H-imidazole synthesized according to step 3 and step 4 of intermediate BB-1. MS (ESI) m/z 412.9 [M+H]⁺.

### Intermediate BB-21. 1-(4-(bromomethyl)phenyl)-3-methoxy-5-methyl-1H-pyrazole

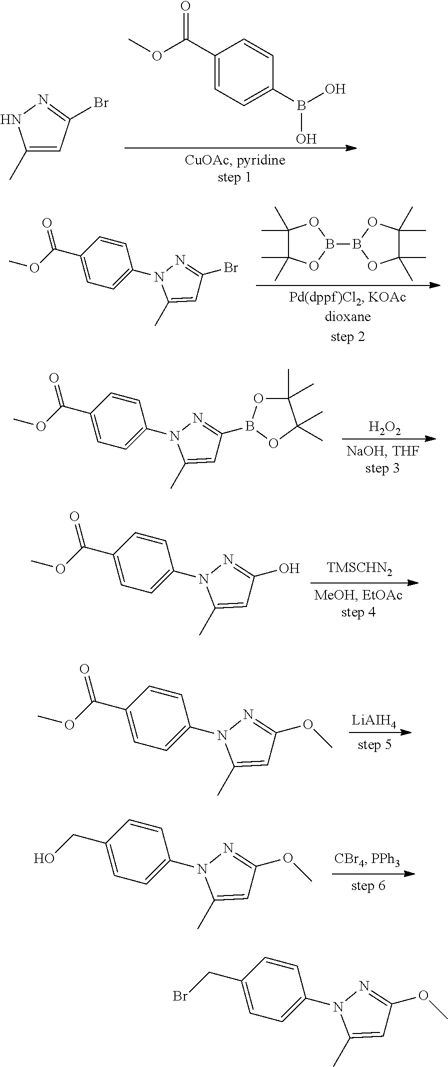

### Step 1: methyl 4-(3-bromo-5-methyl-1H-pyrazol-1-yl)benzoate

A mixture of (4-(methoxycarbonyl)phenyl)boronic acid (1.3 g, 7.45 mmol), 3-bromo-5-methyl-1H-pyrazole (1.0 g, 6.21 mmol), CuOAc (1.5 g, 12.4 mmol) and pyridine (1.0 g, 12.4 mmol) in DCM (10 mL) was stirred at room temperature for overnight. The resulting mixture was filtered through celite, concentrated and purified by silica gel chromatography (eluting with 1/5 EtOAc/PE) to afford methyl 4-(3-bromo-5-methyl-1H-pyrazol-1-yl)benzoate (1.05 g). ¹H NMR (400 MHz, Chloroform-d) δ 8.16-8.09 (m, 2H), 7.56-7.49 (m, 2H), 6.24 (s, 1H), 3.93 (s, 3H), 2.37 (d, J=0.6 Hz, 3H). MS (ESI) m/z 295.0, 297.0 [M+H]⁺.

### Step 2: methyl 4-(5-methyl-3-(4,4,5,5-tetramethyl-1,3,2-dioxaborolan-2-yl)-1H-pyrazol-1-yl)benzoate

A mixture of methyl 4-(3-bromo-5-methyl-1H-pyrazol-1-yl)benzoate (1.0 g, 3.38 mmol), 4,4,4′,4′,5,5,5′,5′-octamethyl-2,2′-bi(1,3,2-dioxaborolane) (2.16 g, 8.48 mmol), KOAc (998 mg, 10.2 mmol) and Pd(dppf)Cl₂ (248 mg, 0.34 mmol) in dioxane (40 mL) was heated at 130° C. for overnight under an atmosphere of N₂. The resulting mixture was filtered through celite, concentrated and purified by silica gel chromatography (eluting with 1/5 MeOH/DCM) to afford methyl 4-(5-methyl-3-(4,4,5,5-tetramethyl-1,3,2-dioxaborolan-2-yl)-1H-pyrazol-1-yl)benzoate (720 mg). MS (ESI) m/z 261.1 [M+H]⁺.

### Step 3: methyl 4-(3-hydroxy-5-methyl-1H-pyrazol-1-yl)benzoate

To a solution of methyl 4-(5-methyl-3-(4,4, 5,5-tetramethyl-1,3 ,2-dioxaborolan-2-yl)-1H-pyrazol-1-yl)benzoate (720 mg, 2.11 mmol) in THF (10 mL) was added NaOH (2 M, 1.5 ml) and H₂O₂ (30%, 3 ml). The resulting solution was stirred at room temperature for 2 h. Solvent was removed in vacuo and the crude residue was purified by silica gel chromatography (eluting with 1/1 EtOAc/PE) to afford methyl 4-(3-hydroxy-5-methyl-1H-pyrazol-1-yl)benzoate (280 mg). MS (ESI) m/z 233.1 [M+H]⁺.

### Step 4: methyl 4-(3-methoxy-5-methyl-1H-pyrazol-1-yl)benzoate

To a solution of methyl 4-(3-hydroxy-5-methyl-1H-pyrazol-1-yl)benzoate (450 mg, 1.94 mmol) in EtOAc (16 mL) and MeOH (1.6 mL) was added TMSCHN₂ (1.6 mL, 3.10 mmol) at 0° C. The resulting solution was stirred at room temperature for overnight. Solvent was removed and the crude was purified by silica gel chromatography (eluting with 1/5 EtOAc/PE) to afford methyl 4-(3-methoxy-5-methyl-1H-pyrazol-1-yl)benzoate (150 mg). MS (ESI) m/z 247.1 [M+H]⁺.

### Step 5 and step 6. 1-(4-(bromomethyl)phenyl)-3-methoxy-5-methyl-1H-pyrazole

1-(4-(bromomethyl)phenyl)-3-methoxy-5-methyl-1H-pyrazole was prepared from methyl 4-(3-methoxy-5-methyl-1H-pyrazol-1-yl)benzoate following step 3 and step 4 of intermediate BB-1. MS (ESI) m/z 281.0, 283.0 [M+H]⁺.

### Intermediate BB-22. 1-(4-(bromomethyl)phenyl)-5-methoxy-3-methyl-1H-pyrazole

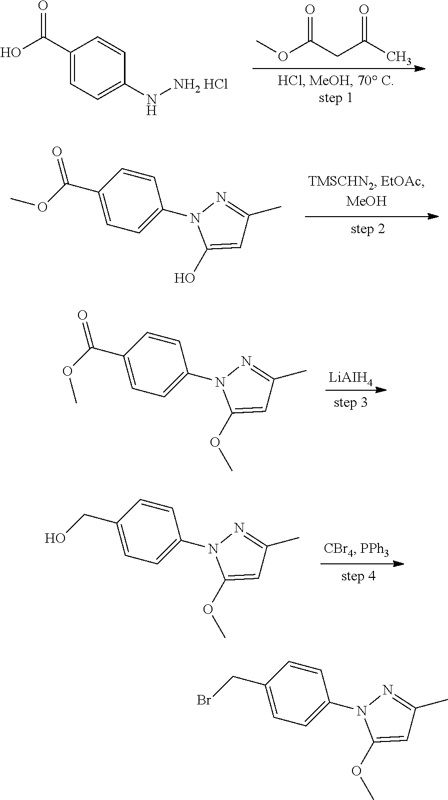

### Step 1. methyl 4-(5-hydroxy-3-methyl-1H-pyrazol-1-yl)benzoate

A mixture of 4-hydrazinylbenzoic acid hydrochloride (5.0 g, 26.5 mmol) and methyl 3-oxobutanoate (4.6 g, 39.8 mmol) in concentrated HCl (6mL) and MeOH (30 mL) was heated at 70° C. for overnight. The pH of the mixture was adjusted to 7-8 with saturated aqueous NaHCO₃. The mixture was extracted with EtOAc (30 mL×3). The organic layers were combined, concentrated under vacuum and purified on a Biotage Isolera One (C₁₈ column, eluting with 10% to 95% MeCN/H₂O) to afford methyl 4-(5-hydroxy-3-methyl-1H-pyrazol-1-yl)benzoate (4.8 g). MS (ESI) m/z 233.1 [M+H]⁺.

### Step 2. methyl 4-(5-methoxy-3-methyl-1H-pyrazol-1-yl)benzoate

To an iced-cooled solution of methyl 4-(5-hydroxy-3-methyl-1H-pyrazol-1-yl)benzoate (1.0 g, 4.31 mmol) in EtOAc (35mL) and MeOH (3.5 mL) was added TMSCHN₂(2 M in hexane, 3.5 ml) over 20 min. Gas evolution was observed and the reaction mixture was stirred for 1 h. The mixture was then allowed to warm to room temperature and stirred for overnight. The resulting mixture was concentrated under vacuum and purified on a Biotage Isolera One (C₁₈ column, eluting with 10% to 95% MeCN/H₂O) to afford methyl 4-(5-methoxy-3-methyl-1H-pyrazol-1-yl)benzoate (720 mg). ¹H NMR (400 MHz, Chloroform-d) δ 8.09-8.02 (m, 2H), 7.86-7.79 (m, 2H), 5.51 (s, 1H), 3.93 (s, 3H), 3.90 (s, 3H), 2.27 (s, 3H). MS (ESI) m/z 247.1 [M+H]⁺.

### Intermediate BB-23. 2-(4-(1-bromoethyl)phenyl)-1-ethyl-4-(trifluoromethyl)-1H-imidazole

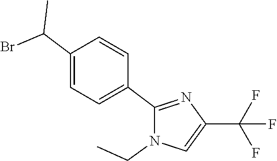 [SMILES: CCn1cc(C(F)(F)F)nc1-c1ccc(C(C)Br)cc1]

2-(4-(1-bromoethyl)phenyl)-1-ethyl-4-(trifluoromethyl)-1H-imidazole was prepared from 4-acetylbenzaldehyde following step 1 to step 4 of Intermediate BB-1. MS (ESI) m/z 347.0, 349.0 [M+H]⁺.

### Intermediate BB-24. 2-(4-(bromomethyl)phenyl)-1-(2-fluoroethyl)-4-(trifluoromethyl)-1H-imidazole

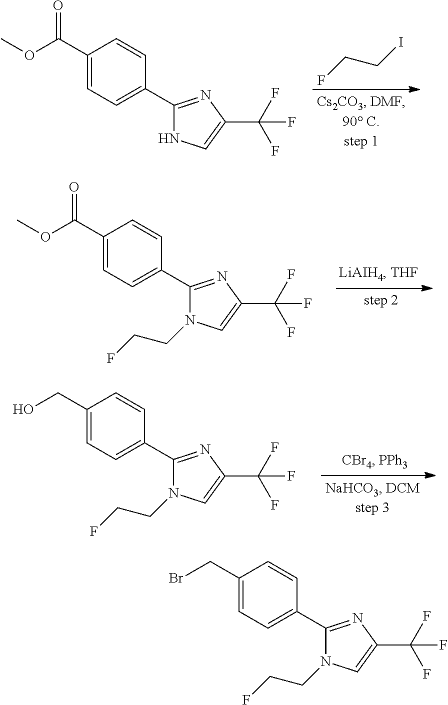

### Step 1. methyl 4-(1-(2-fluoroethyl)-4-(trifluoromethyl)-1H-imidazol-2-yl)benzoate

A mixture of methyl 4-(4-(trifluoromethyl)-1H-imidazol-2-yl)benzoate (10 g, 37 mmol), 2-iodopropane (9.66 g, 55.5 mmol) and Cs₂CO₃ (36.1 g, 111 mmol) in MeCN (100 mL) was heated at 80° C. overnight. The mixture concentrated under vacuum, diluted with water (100 mL) and extracted with EtOAc (3×100 mL). The combined organic layers were dried over anhydrous Na₂SO₄, concentrated under vacuum and purified by silica gel chromatography (eluting with 1/4 EtOAc/PE) to afford methyl 4-(1-isopropyl-4-(trifluoromethyl)-1H-imidazol-2-yl)benzoate (8.6 g). MS (ESI) m/z 317.1 [M+H]⁺.

### Step 2. (4-(1-(2-fluoroethyl)-4-(trifluoromethyl)-1H-imidazol-2-yl)phenyl)methanol

(4-(1-(2-fluoroethyl)-4-(trifluoromethyl)-1H-imidazol-2-yl)phenyl)methanol was prepared from methyl 4-(1-(2-fluoroethyl)-4-(trifluoromethyl)-1H-imidazol-2-yl)benzoate according to step 3 of Intermediate BB-1. MS (ESI) m/z 289.1 [M+H]⁺.

### Step 3. 2-(4-(bromomethyl)phenyl)-1-(2-fluoroethyl)-4-(trifluoromethyl)-1H-imidazole

2-(4-(bromomethyl)phenyl)-1-(2-fluoroethyl)-4-(trifluoromethyl)-1H-imidazole was prepared from (4-(1-(2-fluoroethyl)-4-(trifluoromethyl)-1H-imidazol-2-yl)phenyl)methanol according to step 4 of Intermediate BB-1. MS (ESI) m/z 351.0, 353.0 [M+H]⁺.

### Intermediate BB-25. 5-(4-(bromomethyl)phenyl)-1-ethyl-3-(trifluoromethyl)-1H-1,2,4-triazole

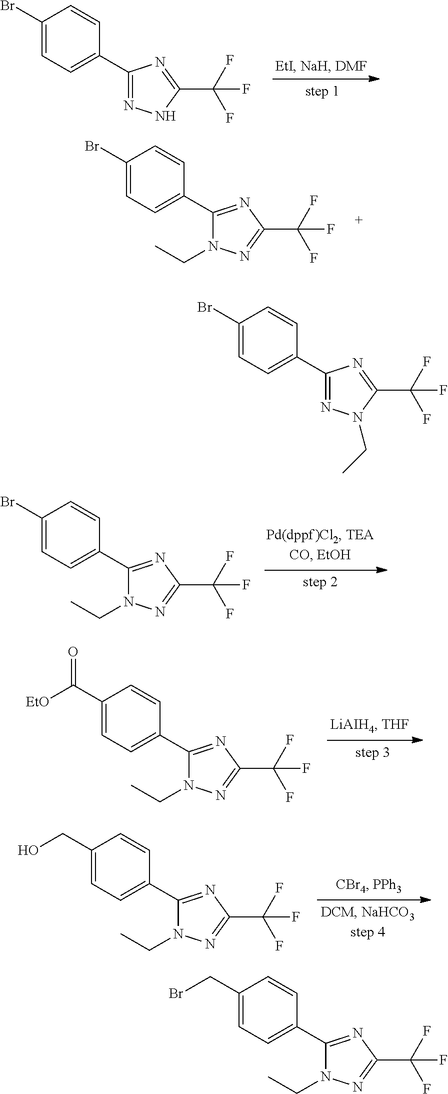

### Step 1. 5-(4-bromophenyl)-1-ethyl-3-(trifluoromethyl)-1H-1,2,4-triazole and 3-(4-bromophenyl)-1-ethyl-5-(trifluoromethyl)-1H-1,2,4-triazole

To a mixture of 3-(4-bromophenyl)-5-(trifluoromethyl)-1H-1,2,4-triazole (8.2 g, 28.1 mmol) in DMF (20 mL) at 0° C. was added portion-wise sodium hydride (2.25 g, 60% in mineral oil, 56.16 mmol). The mixture was stirred for at room temperature 30 min then iodoethane (4.85 g, 30.89 mmol) was added. The resulting solution was stirred at room temperature for 1 h then poured into ice water (50 mL), extracted with EtOAc (3×50 mL). The organic layers were combined, dried over anhydrous sodium sulfate, filtered, concentrated and purified by column chromatography (eluting with a gradient of 0 to 33.3% EtOAc/PE) to give 5-(4-bromophenyl)-1-ethyl-3-(trifluoromethyl)-1H-1,2,4-triazole (1.6 g) and 3-(4-bromophenyl)-1-ethyl-5-(trifluoromethyl)-1H-1,2,4-triazole (4.1 g). MS (ESI) m/z 320.0, 322.0 [M+H]⁺.

### Step 2. Ethyl 4-(1-ethyl-3-(trifluoromethyl)-1H-1,2,4-triazol-5-yl)benzoate

Ethyl 4-(1-ethyl-3-(trifluoromethyl)-1H-1,2,4-triazol-5-yl)benzoate was prepared from 5-(4-bromophenyl)-1-ethyl-3-(trifluoromethyl)-1H-1,2,4-triazole according to step 4 of Intermediate BB-11. ¹H NMR (400 MHz, DMSO-d₆) δ 8.14 (d, J=8.1 Hz, 2H), 7.92 (d, J=8.1 Hz, 2H), 4.40-4.34 (m, 4H), 1.43 (t, J=7.2 Hz, 3H), 1.35 (t, J=7.1 Hz, 3H). MS (ESI) m/z 314.2 [M+H]⁺.

### Step 3. (4-(1-ethyl-3-(trifluoromethyl)-1H-1,2,4-triazol-5-yl)phenyl)methanol

(4-(1-ethyl-5-(trifluoromethyl)-1H-1,2,4-triazol-3-yl)phenyl)methanol was synthesized from ethyl 4-(1-ethyl-3-(trifluoromethyl)-1H-1,2,4-triazol-5-yl)benzoate according to step 3 of Intermediate BB-1. MS (ESI) m/z 272.1 [M+H]⁺.

### Step 4. 5-(4-(bromomethyl)phenyl)-1-ethyl-3-(trifluoromethyl)-1H-1,2,4-triazole

5-(4-(bromomethyl)phenyl)-1-ethyl-3-(trifluoromethyl)-1H-1,2,4-triazole was synthesized from (4-(1-ethyl-3-(trifluoromethyl)-1H-1,2,4-triazol-5-yl)phenyl)methanol according to step 4 of Intermediate BB-1. ¹H NMR (400 MHz, DMSO-d₆) δ 7.77 (d, J=8.0 Hz, 2H), 7.68 (d, J=8.0 Hz, 2H), 4.80 (s, 2H), 4.35 (q, J=7.2 Hz, 2H), 1.42 (t, J=7.2 Hz, 3H). MS (ESI) m/z 334.1, 336.1 [M+H]⁺.

### Intermediate BB-26. 5-(bromomethyl)-2-(1-methyl-4-(trifluoromethyl)-1H-imidazol-2-yl)pyridine

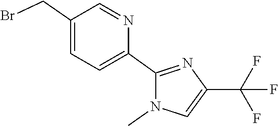 [SMILES: Cn1cc(C(F)(F)F)nc1-c1ccc(CBr)cn1]

5-(bromomethyl)-2-(1-methyl-4-(trifluoromethyl)-1H-imidazol-2-yl)pyridine was prepared from methyl 6-formylnicotinate following step 1 to step 4 of intermediate BB-1. MS (ESI) m/z 320.0, 322.0 [M+H]⁺.

### Intermediate BB-27. 5-(bromomethyl)-2-(1-ethyl-4-(trifluoromethyl)-1H-imidazol-2-yl)pyridine

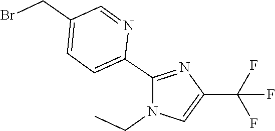 [SMILES: CCn1cc(C(F)(F)F)nc1-c1ccc(CBr)cn1]

5-(bromomethyl)-2-(1-ethyl-4-(trifluoromethyl)-1H-imidazol-2-yl)pyridine was prepared from methyl 6-formylnicotinate following step 1 to step 4 of intermediate BB-1. MS (ESI) m/z 334.0, 336.0 [M+H]⁺.

### Intermediate BB-28. 5-(bromomethyl)-2-(5-methoxy-3-(trifluoromethyl)-1H-pyrazol-1-yl)pyridine

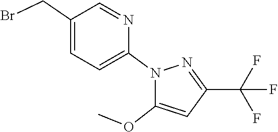 [SMILES: COc1cc(C(F)(F)F)nn1-c1ccc(CBr)cn1]

5-(bromomethyl)-2-(5-methoxy-3-(trifluoromethyl)-1H-pyrazol-1-yl)pyridine was prepared from 6-hydrazinylnicotinic acid following intermediate BB-9. MS (ESI) m/z 336.0, 338.0 [M+H]⁺.

### Intermediate BB-29. 4-(5-methyl-3-(trifluoromethyl)-1H-pyrazol-1-yl)aniline

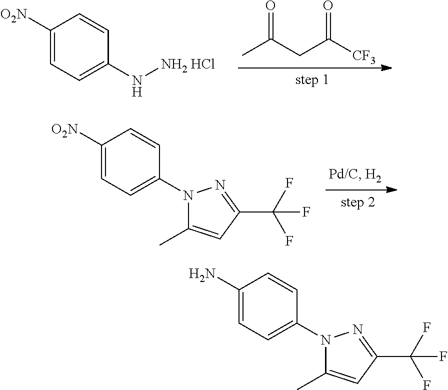

### Step 1. 5-methyl-1-(4-nitrophenyl)-3-(trifluoromethyl)-1H-pyrazole

A solution of (4-nitrophenyl)hydrazine hydrochloride (332 mg, 1.23 mmol) and NaOAc (110 mg, 1.34 mmol) in H₂O (2 mL) was heated at 95° C. for 30 min. After cooled to 0° C., a solution of 1,1,1-trifluoropentane-2,4-dione (200 mg, 1.3 mmol) in ammonium hydroxide solution (28%, 2 mL) and MeOH (6 mL) was added into the above reaction mixture at 0° C. The resulting reaction mixture was stirred for room temperature for 14 h. The reaction mixture was diluted with H₂O, extracted with DCM. The combined organic layers were washed with water and brine, dried over anhydrous Na₂SO₄, filtered and concentrated in vacuo. The crude residue was purified on a Biotage Isolera One (C₁₈ column, eluting with 10% to 90% MeCN/H₂O) to afford 5-methyl-1-(4-nitrophenyl) (trifluoromethyl)-1H-pyrazole (212 mg). MS (ESI) m/z 272.1 [M+H]⁺.

### Step 2. 4-(5-methyl-3-(trifluoromethyl)-1H-pyrazol-1-yl)aniline

To a solution of 5-methyl-1-(4-nitrophenyl)-3-(trifluoromethyl)-1H-pyrazole (570 mg, 2.1 mmol) in MeOH (20 mL) was added 5% Pd/C (300 mg). The reaction mixture was stirred at room temperature overnight under H₂ atmosphere. The reaction mixture was then filtered through celite. The filtrate was concentrated under vacuum to afford 4-(5-methyl-3-(trifluoromethyl)-1H-pyrazol-1-yl)aniline (480 mg). MS (ESI) m/z 242.1 [M+H]⁺.

### Intermediate BB-30. 2-(4-(bromomethyl)-2-methoxyphenyl)-1-isopropyl-4-(trifluoromethyl)-1H-imidazole

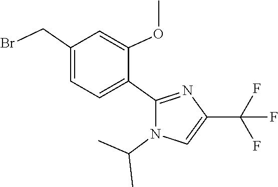 [SMILES: COc1cc(CBr)ccc1-c1nc(C(F)(F)F)cn1C(C)C]

2-(4-(bromomethyl)-2-methoxyphenyl)-1-isopropyl-4-(trifluoromethyl)-1H-imidazole was prepared from 4-formyl-3-methoxybenzonitrile following Intermediate BB-4. MS (ESI) m/z 277.0, 279.0 [M+H]⁺.

### Intermediate BB-31. 4-(4-(bromomethyl)phenyl)-1-methyl-2-(trifluoromethyl)-1H-imidazole

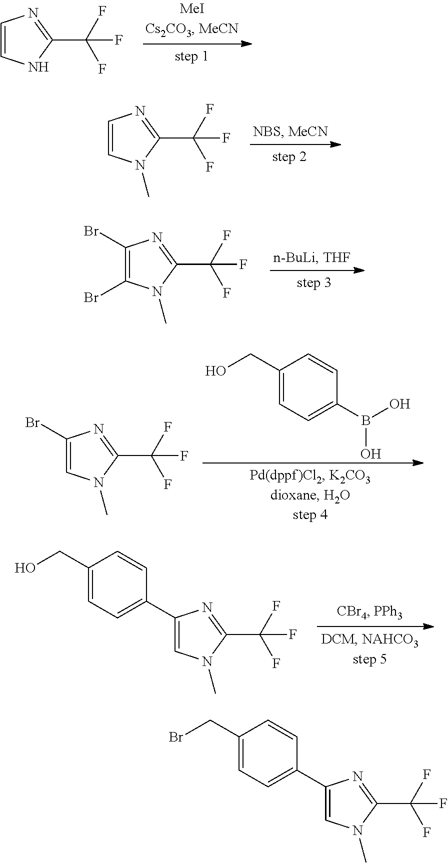

### Step 1. 1-methyl-2-(trifluoromethyl)-1H-imidazole

1-methyl-2-(trifluoromethyl)-1H-imidazole was prepared from 2-(trifluoromethyl)-1H-imidazole and iodomethane according to step 1 of intermediate BB-3. MS (ESI) m/z 151.0 [M+H]⁺.

### Step 2. 4,5-dibromo-1-methyl-2-(trifluoromethyl)-1H-imidazole

To a stirred solution of 1-methyl-2-(trifluoromethyl)-1H-imidazole (540 mg, 3.6 mmol) in MeCN (10 mL) was added NBS (1.92 g, 10.8 mmol) at room temperature. The resulting mixture was stirred at room temperature for 3 h. The solvent was removed in vacuo and the crude residue was purified by column chromatography (eluting with a gradient of 0 to 33.3% EtOAc/PE) to give 4,5-dibromo-1-methyl-2-(trifluoromethyl)-1H-imidazole (390 mg). MS (ESI) m/z 306.9, 308.9, 310.9 [M+H]⁺.

### Step 3. 4-bromo-1-methyl-2-(trifluoromethyl)-1H-imidazole

To a stirred solution of 4,5-dibromo-1-methyl-2-(trifluoromethyl)-1H-imidazole (360 mg, 1.17 mmol) in dry THF (3 mL) was added n-BuLi (2.5 M in hexane, 0.52 mL, 1.29 mmol) dropwise at −78° C. After addition, the resulting solution was stirred at −60° C. for 1 h then warmed to room temperature and quenched with aqueous saturated NH₄Cl (20 mL). The mixture was extracted with EtOAc (15 mL×3), dried over anhydrous Na₂SO₄, concentrated and purified by column chromatography (eluting with a gradient of 0 to 33.3% EtOAc/PE) to give 4-bromo-1-methyl-2-(trifluoromethyl)-1H-imidazole (220 mg). ¹H NMR (400 MHz, Chloroform-d) δ 6.97 (s, 1H), 3.77 (d, J=1.1 Hz, 3H).

### Step 4. (4-(1-methyl-2-(trifluoromethyl)-1H-imidazol-4-yl)phenyl)methanol

(4-(1-methyl-2-(trifluoromethyl)-1H-imidazol-4-yl)phenyl)methanol was synthesized from 4-bromo-1-methyl-2-(trifluoromethyl)-1H-imidazole and (4-(hydroxymethyl)phenyl)boronic acid following step 1 of Intermediate BB-16. MS (ESI) m/z 257.1 [M+H]⁺.

### Step 5. 4-(4-(bromomethyl)phenyl)-1-methyl-2-(trifluoromethyl)-1H-imidazole

4-(4-(bromomethyl)phenyl)-1-methyl-2-(trifluoromethyl)-1H-imidazole was synthesized from (4-(1-methyl-2-(trifluoromethyl)-1H-imidazol-4-yl)phenyl)methanol according to step 4 of intermediate BB-1. MS (ESI) m/z 319.0, 321.0 [M+H]⁺.

### Intermediate BB-32. 2-(4-(bromomethyl)phenyl)-1-isopropyl-4-nitro-1H-imidazole

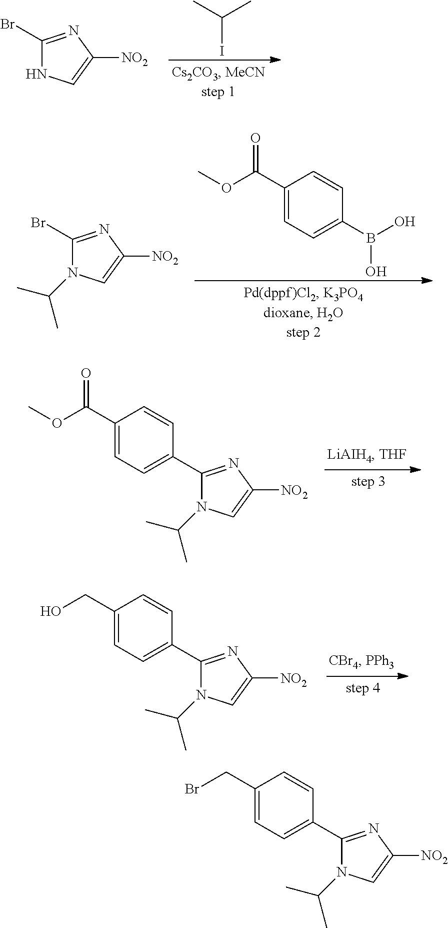

### Step 1. 2-bromo-1-isopropyl-4-nitro-1H-imidazole

2-bromo-1-isopropyl-4-nitro-1H-imidazole was prepared from 2-bromo-4-nitro-1H-imidazole according to step 1 of Intermediate BB-3. MS (ESI) m/z 234.0, 236.0 [M+H]⁺.

### Step 2 to step 4. 2-(4-(bromomethyl)phenyl)-1-isopropyl-4-nitro-1H-imidazole

2-(4-(bromomethyl)phenyl)-1-isopropyl-4-nitro-1H-imidazole was prepared from 2-bromo-1-isopropyl-4-nitro-1H-imidazole following step 1 to step 3 of Intermediate BB-16. ¹H NMR (400 MHz, Chloroform-d) δ 7.94 (s, 1H), 7.59-7.48 (m, 4H), 4.62 (m, 1H), 4.53 (s, 2H), 1.51 (d, J=6.7 Hz, 6H). MS (ESI) m/z 324.0, 326.0 [M+H]⁺.

### Intermediate BB-33. 6-(1-ethyl-4-(trifluoromethyl)-1H-imidazol-2-yl)pyridin-3-amine

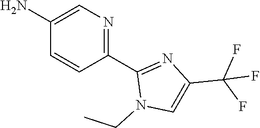 [SMILES: CCn1cc(C(F)(F)F)nc1-c1ccc(N)cn1]

6-(1-ethyl-4-(trifluoromethyl)-1H-imidazol-2-yl)pyridin-3-amine was prepared from 5-nitropicolinaldehyde following Intermediate BB-14. MS (ESI) m/z 257.1 [M+H]⁺.

### Intermediate BB-34. 4-(1-ethyl-4-(trifluoromethyl)-1H-imidazol-2-yl)-3-methoxyaniline

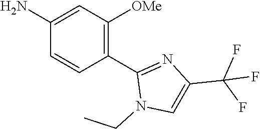 [SMILES: CCn1cc(C(F)(F)F)nc1-c1ccc(N)cc1OC]

4-(1-ethyl-4-(trifluoromethyl)-1H-imidazol-2-yl)-3-methoxyaniline was prepared from 2-methoxy-4-nitrobenzaldehyde following Intermediate BB-14. MS (ESI) m/z 286.1

### Intermediate BB-35. 3-(4-(bromomethyl)phenyl)-1-ethyl-5-(trifluoromethyl)-1H-1,2,4-triazole

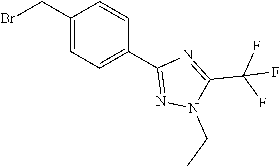 [SMILES: CCn1nc(-c2ccc(CBr)cc2)nc1C(F)(F)F]

3-(4-(bromomethyl)phenyl)-1-ethyl-5-(trifluoromethyl)-1H-1,2,4-triazole was synthesized from 3-(4-bromophenyl)-1-ethyl-5-(trifluoromethyl)-1H-1,2,4-triazole (prepared in step 1 of Intermediate BB-25) following step 4 to step 6 of Intermediate BB-11. MS (ESI) m/z 334.1, 336.1 [M+H]⁺.

### Intermediate BB-36. 1-(4-(bromomethyl)-2-fluorophenyl)-3-methyl-5-(trifluoromethyl)-1H-pyrazole

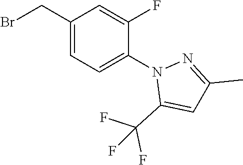 [SMILES: Cc1cc(C(F)(F)F)n(-c2ccc(CBr)cc2F)n1]

1-(4-(bromomethyl)-2-fluorophenyl)-3-methyl-5-(trifluoromethyl)-1H-pyrazole was prepared from ethyl 3-fluoro-4-(3-methyl-5-(trifluoromethyl)-1H-pyrazol-1-yl)benzoate (prepared in step 2 of intermediate BB-8) according to step 3 and step 4 of intermediate BB-1. MS (ESI) m/z 337.0, 339.0 [M+H]⁺.

### Intermediate BB-37. 1-(4-(bromomethyl)phenyl)-5-methoxy-3-(trifluoromethyl)-1H-pyrazole

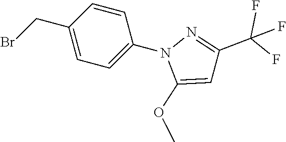 [SMILES: COc1cc(C(F)(F)F)nn1-c1ccc(CBr)cc1]

1-(4-(bromomethyl)phenyl)-5-methoxy-3-(trifluoromethyl)-1H-pyrazole was prepared from methyl 4-(5-hydroxy-3-(trifluoromethyl)-1H-pyrazol-1-yl)benzoate (prepared from step 1 of intermediate BB-9) and iodoethane following step 2 to step 4 of intermediate BB-9. MS (ESI) m/z 349.0, 351.0 [M+H]⁺.

### Intermediate BB-38. 1-(4-(bromomethyl)phenyl)-5-methoxy-1H-pyrazole

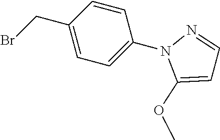

1-(4-(bromomethyl)phenyl)-5-methoxy-1H-pyrazole was prepared from 4-hydrazinylbenzoic acid hydrochloride and methyl 3,3-dimethoxypropanoate following Intermediate BB-9. MS (ESI) m/z 267.0, 269.0 [M+H]⁺.

### Intermediate BB-39. 2-(4-(bromomethyl)phenyl)-4-chloro-1-ethyl-1H-imidazole

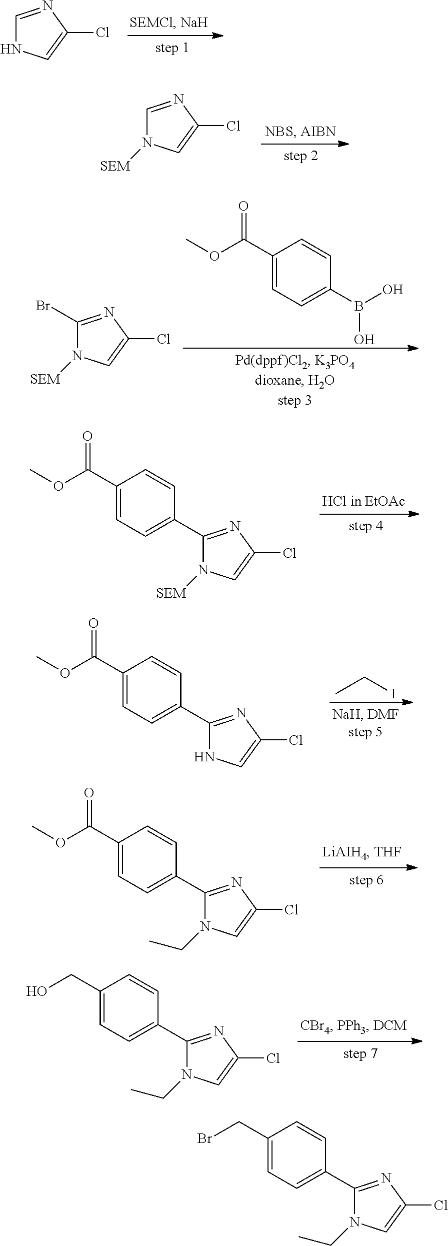

### Step 1. 4-chloro-1((2-(trimethylsilyl)ethoxy)methyl)-1H-imidazole

To a stirred solution of 4-chloro-1H-imidazole (2 g, 19.51 mmol) in dry THF (20 mL) was added 60% NaH (0.86 g, 60% in mineral oil, 21.46 mmol) at 0° C. The resulting mixture was stirred at the same temperature for 30 min then 2-(Trimethylsilyl)ethoxymethyl chloride (3.58 g, 21.46 mmol) was added dropwise. The mixture was stirred at room temperature for 4 h then quenched with ice water (30 mL) and extracted with EtOAc (30 mL×2). The combined organic phases were dried over anhydrous Na₂SO₄, concentrated and purified on a Biotage Isolera One (C₁₈ column, eluting with 10% to 95% MeCN/H₂O containing 0.1% formic acid) to afford 4-chloro-1-((2-(trimethylsilyl)ethoxy)methyl)-1H-imidazole (3.49 g). ¹H NMR (400 MHz, DMSO-d₆) δ 7.82 (s, 1H), 7.43 (s, 1H), 5.32 (s, 2H), 3.51 (t, J=8.0 Hz, 2H), 0.88 (t, J=8.1 Hz, 2H), 0.00 (s, 9H).

### Step 2. 2-bromo-4-chloro-1-((2-(trimethylsilyl)ethoxy)methyl)-1H-imidazole

A mixture of 4-chloro-1((2-(trimethylsilyl)ethoxy)methyl)-1H-imidazole (2 g, 8.58 mmol), NBS (1.53 g, 8.58 mmol) and AIBN (140.9 mg, 0.85 mmol) in CCl₄ (20 mL) was heated at 80° C. for 2 h. The solvent was removed under reduced pressure and the crude purified on a Biotage Isolera One (C₁₈ column, eluting with 10% to 95% MeCN/H₂O containing 0.1% formic acid) to afford 2-bromo-4-chloro-1-((2-(trimethylsilyl)ethoxy)methyl)-1H-imidazole (1.47 g). ¹H NMR (400 MHz, DMSO-d₆) δ 7.69 (s, 1H), 5.29 (s, 2H), 3.57 (t, J=8.0 Hz, 2H), 0.89 (t, J=8.0 Hz, 2H), 0.00 (s, 9H).

### Step 3. methyl 4-(4-chloro-1-((2-(trimethylsilyl)ethoxy)methyl)-1H-imidazol-2-yl)benzoate

Methyl 4-(4-chloro-1-((2-(trimethylsilyl)ethoxy)methyl)-1H-imidazol-2-yl)benzoate was synthesized from 2-bromo-4-chloro-1-((2-(trimethylsilyl)ethoxy)methyl)-1H-imidazole following step 1 of Intermediate BB-16. ¹H NMR (400 MHz, Chloroform-d) δ 8.10 (d, J=8.3 Hz, 2H), 7.87 (d, J=8.3 Hz, 2H), 7.04 (s, 1H), 5.23 (s, 2H), 3.93 (s, 3H), 3.61 (t, J=8.0 Hz, 2H), 0.94 (t, J=8.0 Hz, 2H), 0.00 (s, 9H). MS (ESI) m/z 367.1 [M+H]⁺.

### Step 4. methyl 4-(4-chloro-1H-imidazol-2-yl)benzoate

A mixture of methyl 4-(4-chloro-1-((2-(trimethylsilyl)ethoxy)methyl)-1H-imidazol-2-yl)benzoate (1 g, 2.73 mmol) in HCl-EtOAc solution (4M, 20 mL) was stirred at room temperature for 12 h. The pH of the resulting mixture was adjusted to 8 with saturated Na₂CO₃ solution, extracted with EtOAc (30 mL×3), dried over anhydrous Na₂SO₄ and concentrated under vacuum to afford methyl 4-(4-chloro-1H-imidazol-2-yl)benzoate (640 mg). MS (ESI) m/z 237.0 [M+H]⁺.

### Step 5 to step 7. 2-(4-(bromomethyl)phenyl)-4-chloro-1-ethyl-1H-imidazole

2-(4-(bromomethyl)phenyl)-4-chloro-1-ethyl-1H-imidazole was prepared from methyl 4-(4-chloro-1H-imidazol-2-yl)benzoate following step 2 to step 4 of intermediate BB-1. MS (ESI) m/z 298.9, 300.9 [M+H]⁺.

### Intermediate BB-40. 2-(4-(bromomethyl)-2-(2-methoxyethoxy)phenyl)-1-isopropyl (trifluoromethyl)-1H-imidazole

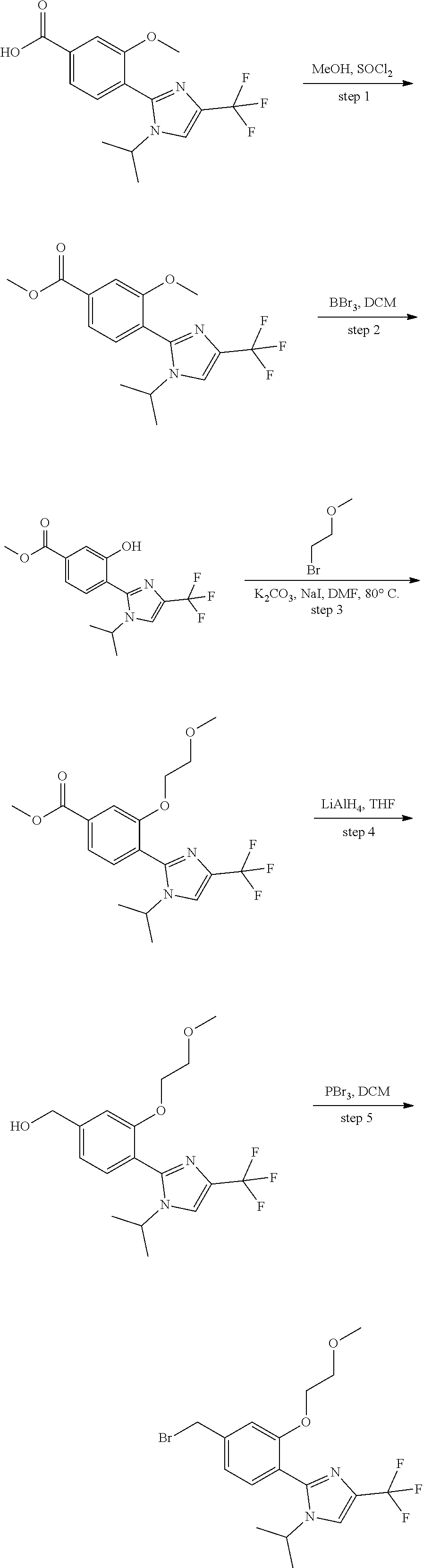

### Step 1. methyl 4-(1-isopropyl-4-(trifluoromethyl)-1H-imidazol-2-yl)-3-methoxybenzoate

To a solution of 4-(1-isopropyl-4-(trifluoromethyl)-1H-imidazol-2-yl)-3-methoxybenzoic acid (prepared from 4-formyl-3-methoxybenzonitrile following Intermediate BB-4, 3.28 g, 10 mmol) in MeOH (50 mL) was added dropwise thionyl chloride (5 mL) at 0° C. After addition, the solution was heated at 70° C. for 3 h. Solvent was removed and the crude was purified on a Biotage Isolera One (C₁₈ column, eluting with 30% to 90% MeCN/H₂O, containing 0.1% TFA) to afford methyl 4-(1-isopropyl-4-(trifluoromethyl)-1H-imidazol-2-yl)-3-methoxybenzoate (2.9 g). MS (ESI) m/z 343.1 [M+H]⁺.

### Step 2. methyl 4-(1-isopropyl-4-(trifluoromethyl)-1H-imidazol-2-yl)-3-methoxybenzoate

A solution of methyl 4-(1-isopropyl-4-(trifluoromethyl)-1H-imidazol-2-yl)-3-methoxybenzoate (1.96 g, 5.73 mmol) in DCM (15 mL) was added BBr₃ (1 M in DCM, 23 mL) dropwise at 0° C. The resulting solution was stirred at the same temperature for 2 h. The reaction mixture was quenched with MeOH (30 mL) and concentrated under vacuum. The crude product was purified by Biotage Isolera One (C₁₈ column, eluting with 30% to 90% MeCN/H₂O, containing 0.1% TFA) to afford methyl 3-hydroxy-4-(1-isopropyl-4-(trifluoromethyl)-1H-imidazol-2-yl)benzoate (1.45 g). MS (ESI) m/z 329.1 [M+H]⁺.

### Step 3. methyl 4-(1-isopropyl-4-(trifluoromethyl)-1H-imidazol-2-yl)-3-(2-methoxyethoxy)benzoate

A mixture of methyl 3-hydroxy-4-(1-isopropyl-4-(trifluoromethyl)-1H-imidazol yl)benzoate (700 mg, 2.13 mmol), 1-bromo-2-methoxyethane (592 mg, 4.26 mmol), K₂CO₃ (883 mg, 6.39 mmol) and NaI (160 mg, 1.07 mmol) in DMF (10 mL) was heated at 80° C. for 2 h. The solution was diluted with water (30 mL) and extracted with EtOAc (30 mL×3). The combined organic layers were concentrated under vacuum and purified by silica gel chromatography (eluting with 1/6 EtOAc/PE) to afford methyl 4-(1-isopropyl-4-(trifluoromethyl)-1H-imidazol-2-yl)-3-(2-methoxyethoxy)benzoate (520 mg). MS (ESI) m/z 387.1 [M+H]⁺.

### Step 4 and step 5. 2-(4-(bromomethyl)-2-(2-methoxyethoxy)phenyl)-1-isopropyl-4-(trifluoromethyl)-1H-imidazole

2-(4-(bromomethyl)-2-(2-methoxyethoxy)phenyl)-1-isopropyl-4-(trifluoromethyl)-1H-imidazole was prepared from methyl 4-(1-isopropyl-4-(trifluoromethyl)-1H-imidazol-2-yl)-3-(2-methoxyethoxy)benzoate following step 3 and step 4 of intermediate BB-1. MS (ESI) m/z 421.1, 423.1 [M+H]⁺.

### Intermediate BB-41. 2-(4-(bromomethyl)-2-(2-((tert-butyldimethylsilyl)oxy)ethoxy)phenyl)-1-isopropyl-4-(trifluoromethyl)-1H-imidazole

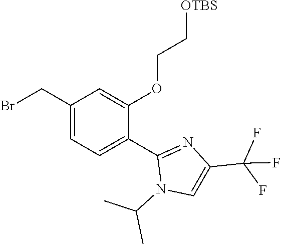 [SMILES: CC(C)n1cc(C(F)(F)F)nc1-c1ccc(CBr)cc1OCCO[Si](C)(C)C(C)(C)C]

2-(4-(bromomethyl)-2-(2-((tert-butyldimethylsilyl)oxy)ethoxy)phenyl)-1-isopropyl-4-(trifluoromethyl)-1H-imidazole was prepared from methyl 3-hydroxy-4-(1-isopropyl-4-(trifluoromethyl)-1H-imidazol-2-yl)benzoate (synthesized in step 2 of intermediate BB-40) and (2-bromoethoxy)(tert-butyl)dimethylsilane according to Intermediate BB-40. MS (ESI) m/z 521.1, 523.1 [M+H]⁺.

### Intermediate BB-42. 4-(4-(bromomethyl)-2-fluorophenyl)-1-methyl-2-(trifluoromethyl)-1H-imidazole

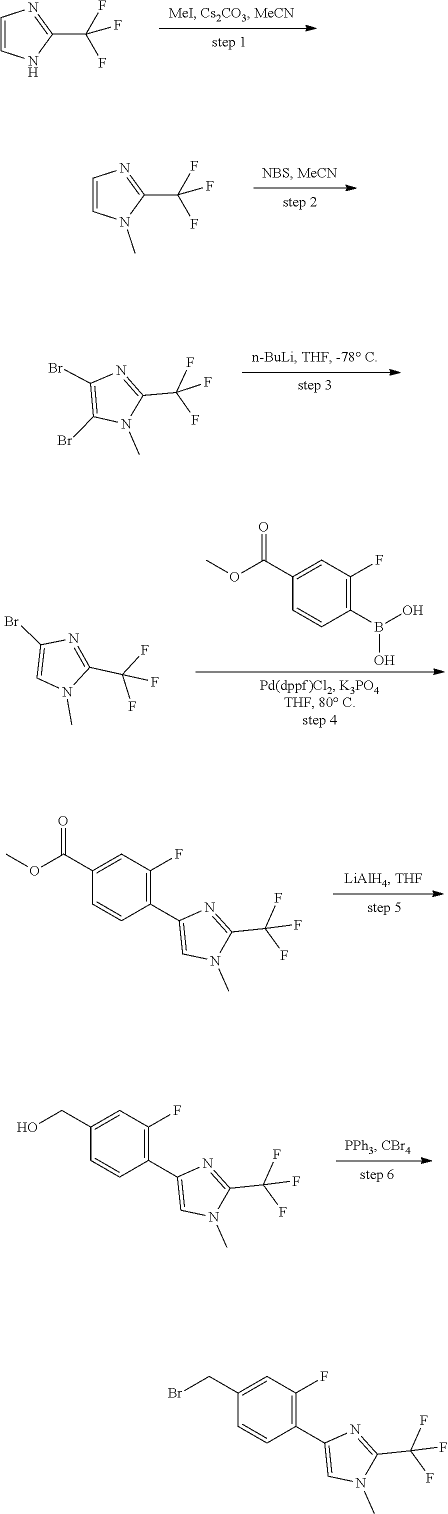

### Step 1. 1-methyl-2-(trifluoromethyl)-1H-imidazole

To a solution of 2-(trifluoromethyl)-1H-imidazole (3.0 g, 22.1 mmol) in MeCN (100 mL), was added MeI (3.4 g, 24.3 mmol) and Cs₂CO₃ (14.3 g, 44.0 mmol). The mixture was heated at 60° C. for 12 h, then filtered through celite. The filtrate was concentrated under vacuum to afford 1-methyl-2-(trifluoromethyl)-1H-imidazole (3.3 g). MS (ESI) m/z 151.0 [M+H]⁺.

### Step 2. 4,5-dibromo-1-methyl-2-(trifluoromethyl)-1H-imidazole

To a solution of 1-methyl-2-(trifluoromethyl)-1H-imidazole (3.3 g, 22.0 mmol) in MeCN (100 mL) was added NBS (11.7 g, 66.0 mmol). The mixture was stirred at room temperature for 12 hoh, then filtered through celite. The filtrate was concentrated under vacuum. The crude product was purified by silica gel chromatography (eluting with 1/6 EtOAc/PE) to afford 4,5-dibromo-1-methyl-2-(trifluoromethyl)-1H-imidazole (4.4 g). MS (ESI) m/z 306.9 [M+H]⁺.

### Step 3. 4-bromo-1-methyl-2-(trifluoromethyl)-1H-imidazole

To a solution of 4,5-dibromo-1-methyl-2-(trifluoromethyl)-1H-imidazole (4.2 g, 13.6 mmol) in dry THF (40 ml) was added n-BuLi (2.5 M in THF, 6 mL) slowly at −78° C. The mixture was stirred at −78° C. for 1 h, then warmed to room temperature and quenched with saturated aqueous NH₄Cl (50 mL) and extracted with EtOAc (100 mL×3). The combined organic layers were washed with brine, dried over Na₂SO₄, concentrated and purified by silica gel chromatography (eluting with 1/6 EtOAc/PE) to afford 4-bromo-1-methyl-2-(trifluoromethyl)-1H-imidazole (1.85 g). MS (ESI) m/z 229.0, 231.0[M+H]⁺.

### Step 4. methyl 3-fluoro-4-(1-methyl-2-(trifluoromethyl)-1H-imidazol-4-yl)benzoate

To a solution of 4-bromo-1-methyl-2-(trifluoromethyl)-1H-imidazole (1.61 g, 7.0 mmol) in THF (60 mL) was added (2-fluoro-4-(methoxycarbonyl)phenyl)boronic acid (4.2 g, 21.1 mmol), Pd(dppf)Cl₂ (513.0 mg, 0.7 mmol) and K₃PO₄ (4.5 g, 21.1 mmol). The mixture was heated at 80° C. for 12 h under N₂. The resulting mixture was filtered through celite and the filtrate was concentrated under vacuum. The crude product was purified by silica gel chromatography (eluting with 1/10 EtOAc/PE) to afford methyl 3-fluoro-4-(1-methyl-2-(trifluoromethyl)-1H-imidazol-4-yl)benzoate (1.9 g). MS (ESI) m/z 303.1 [M+H]⁺.

### Step 5 and Step 6. 4-(4-(bromomethyl)-2-fluorophenyl)-1-methyl-2-(trifluoromethyl)-1H-imidazole

4-(4-(bromomethyl)-2-fluorophenyl)-1-methyl-2-(trifluoromethyl)-1H-imidazole was prepared from methyl 3-fluoro-4-(1-methyl-2-(trifluoromethyl)-1H-imidazol-4-yl)benzoate following step 3 and step 4 of intermediate BB-1. ¹H NMR (400 MHz, Chloroform-d) δ 8.10 (t, J=8.0 Hz, 1H), 7.48 (s, 1H), 7.41 (d, J=3.7 Hz, 1H), 7.18 (dd, J=8.0, 1.7 Hz, 1H), 7.08 (dd, J=11.7, 1.7 Hz, 1H), 6.14 (s, 1H), 5.15 (m, 1H), 4.99 (s, 2H), 4.43 (t, J=7.1 Hz, 2H), 3.87-3.78 (m, 3H), 3.10 (t, J=7.1 Hz, 2H), 1.44 (d, J=6.6 Hz, 6H).

### Intermediate BB-43. 2-(4-(bromomethyl)-2-chlorophenyl)-1-isopropyl-4-(trifluoromethyl)-1H-imidazole

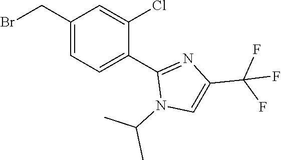 [SMILES: CC(C)n1cc(C(F)(F)F)nc1-c1ccc(CBr)cc1Cl]

2-(4-(bromomethyl)-2-chlorophenyl)-1-isopropyl-4-(trifluoromethyl)-1H-imidazole was prepared from methyl 3-chloro-4-formylbenzoate following Intermediate BB-1. MS (ESI) m/z 381.0, 383.0 [M+H]⁺.

### Intermediate BB-44. 3-(4-(bromomethyl)phenyl)-1-methyl-1H-pyrazole-4-carbonitrile

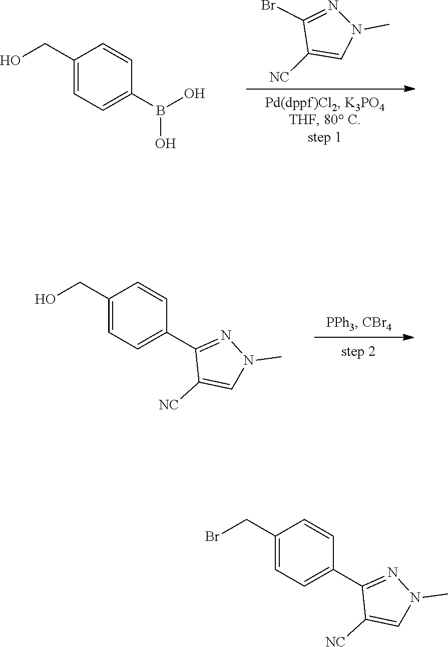

### Step 1. 3-(4-(hydroxymethyl)phenyl)-1-methyl-1H-pyrazole-4-carbonitrile

To a solution of 3-bromo-1-methyl-1H-pyrazole-4-carbonitrile (500 mg, 2.7 mmol) in THF (15 mL) was added (4-(hydroxymethyl)phenyl)boronic acid (1.2 g, 8.1 mmol), K₃PO₄ (1.7 g, 8.1 mmol) and Pd(dppf)Cl₂ (200 mg, 0.3 mmol). The mixture was heated at 80° C. for overnight under N₂. The solution was filtered through celite and the filtrate was concentrated under vacuum. The crude product was purified by silica gel chromatography (eluting with 1/2 EtOAc/PE) to afford 3-(4-(hydroxymethyl)phenyl)-1-methyl-1H-pyrazole-4-carbonitrile (475.0 mg). MS (ESI) m/z 214.1 [M+H]⁺.

### Step 2. 3-(4-(bromomethyl)phenyl)-1-methyl-1H-pyrazole-4-carbonitrile

3-(4-(bromomethyl)phenyl)-1-methyl-1H-pyrazole-4-carbonitrile was prepared according to step 4 of intermediate BB-1. MS (ESI) m/z 276.0, 278.0 [M+H]⁺.

### Intermediate BB-45. 2-(4-(bromomethyl)-2-chlorophenyl)-1-ethyl-4-(trifluoromethyl)-1H-imidazole

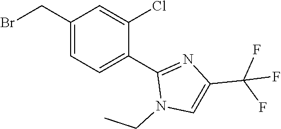 [SMILES: CCn1cc(C(F)(F)F)nc1-c1ccc(CBr)cc1Cl]

2-(4-(bromomethyl)-2-chlorophenyl)-1-ethyl-4-(trifluoromethyl)-1H-imidazole was prepared from methyl 3-chloro-4-formylbenzoate following Intermediate BB-1. MS (ESI) m/z 367.0, 369.0 [M+H]⁺.

### Intermediate BB-46. 2-(4-(bromomethyl)-2-fluoro-6-methoxyphenyl)-1-ethyl-4-(trifluoromethyl)-1H-imidazole

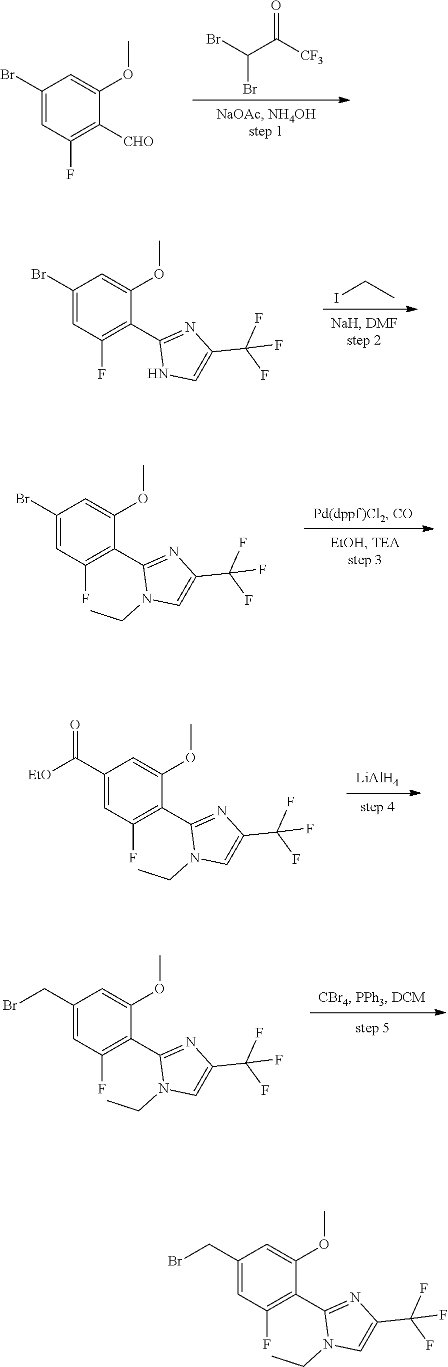

### Step 1 and step 2. 2-(4-bromo-2-fluoro-6-methoxyphenyl)-1-ethyl-4-(trifluoromethyl)-1H-imidazole

2-(4-bromo-2-fluoro-6-methoxyphenyl)-1-ethyl-4-(trifluoromethyl)-1H-imidazole was prepared from 4-bromo-2-fluoro-6-methoxybenzaldehyde following steps 1 and 2 of intermediate BB-1. MS (ESI) m/z 367.0, 369.0 [M+H]⁺.

### Step 3 to step 5. 2-(4-(bromomethyl)-2-fluoro-6-methoxyphenyl)-1-ethyl-4-(trifluoromethyl)-1H-imidazole

2-(4-(bromomethyl)-2-fluoro-6-methoxyphenyl)-1-ethyl-4-(trifluoromethyl)-1H-imidazole was prepared from 2-(4-bromo-2-fluoro-6-methoxyphenyl)-1-ethyl-4-(trifluoromethyl)-1H-imidazole according to step 4 to step 6 of Intermediate BB-11. MS (ESI) m/z 381.0, 383.0 [M+H]⁺.

### Intermediate BB-47. 2-(4-(bromomethyl)phenyl)-1-ethyl-4-methyl-1H-imidazole

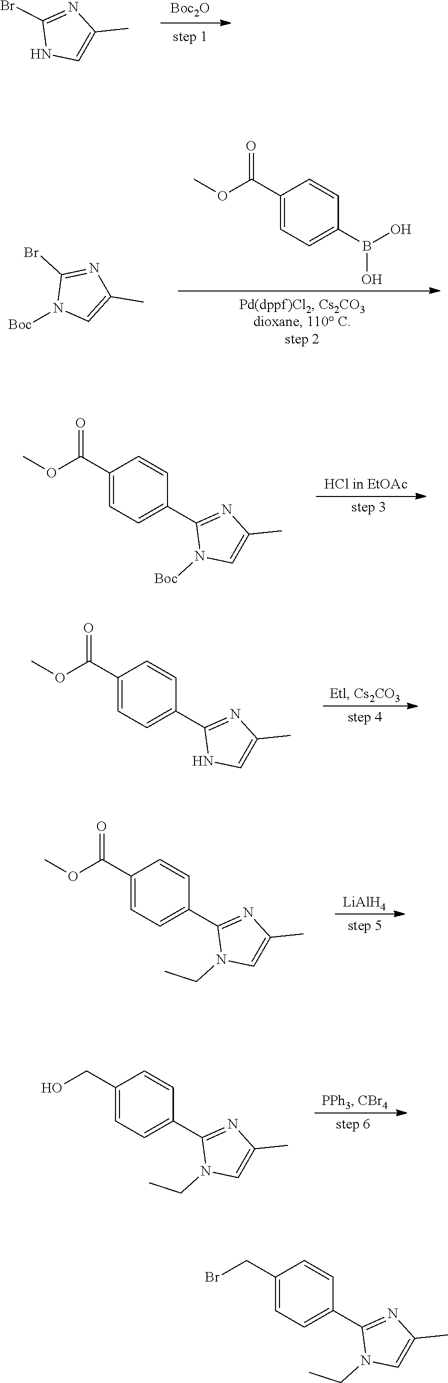

### Step 1. tert-butyl 2-bromo-4-methyl-1H-imidazole-1-carboxylate

The solution of 2-bromo-4-methyl-1H-imidazole (3.2 g, 20.0 mmol) in DCM (12 mL) and TEA (4 ml) was added Boc₂O (13.1 g, 60.0 mmol) and DMAP (488.0 mg, 4.0 mmol). The mixture was stirred at room temperature for 1 h. The resulting mixture was concentrated under vacuum. The crude product was purified by silica gel chromatography (eluting with 1/5 EtOAc/PE) to afford tert-butyl 2-bromo-4-methyl-1H-imidazole carboxylate (5.2 g). MS (ESI) m/z 261.0 263.0 [M+H]⁺.

### Step 2. tert-butyl 2-(4-(methoxycarbonyl)phenyl)-4-methyl-1H-imidazole-1-carboxylate

The solution of tert-butyl 2-bromo-4-methyl-1H-imidazole-1-carboxylate (4.0 g, 15.4 mmol) in dioxane (30 mL) was added (4-(methoxycarbonyl)phenyl)boronic acid (11.1 g 61.5 mmol), Pd(dppf)Cl₂(1.1 g 1.5 mmol) and Cs₂CO₃ (10.0 g, 30.8 mmol). The mixture was heated at 110° C. for 12 h under N₂. The solution was filtered through celite and the filtrate was concentrated under vacuum. The crude product was purified by silica gel chromatography (eluting with 1/5 EtOAc/PE) to afford tert-butyl 2-(4-(methoxycarbonyl)phenyl)-4-methyl-1H-imidazole-1-carboxylate (3.5 g). MS (ESI) m/z 317.1 [M+H]⁺.

### Step 3. methyl 4-(4-methyl-1H-imidazol-2-yl)benzoate

To a solution of tert-butyl 2-(4-(methoxycarbonyl)phenyl)-4-methyl-1H-imidazole-1-carboxylate (3.5 g, 11.2 mmol) in DCM (20 mL) was added TFA (20 mL) slowly at 0° C. The mixture was then stirred at room temperature for 2 h. The resulting mixture was concentrated under vacuum to afford methyl 4-(4-methyl-1H-imidazol-2-yl)benzoate (3.0 g). MS (ESI) m/z 217.1 [M+H]⁺.

### Step 4. methyl 4-(1-ethyl-4-methyl-1H-imidazol-2-yl)benzoate

To a solution of methyl 4-(4-methyl-1H-imidazol-2-yl)benzoate (3.0 g, 14 mmol) in MeCN (25 mL) was added EtI (4.4 g, 28.1 mmol) and Cs₂CO₃ (9.1 g, 28.1 mmol). The mixture was heated at 80° C. for 2 h. The solution was filtered through celite and the filtrate was concentrated under vacuum. The crude product was directly purified by silica gel chromatography (eluting with 1:2 EtOAc/PE) to afford methyl 4-(1-ethyl-4-methyl-1H-imidazol-2-yl)benzoate (707.0 mg). MS (ESI) m/z 245.1 [M+H]⁺.

### Step 5 and step 6. 2-(4-(bromomethyl)phenyl)-1-ethyl-4-methyl-1H-imidazole

2-(4-(bromomethyl)phenyl)-1-ethyl-4-methyl-1H-imidazole was synthesized from methyl 4-(1-ethyl-4-methyl-1H-imidazol-2-yl)benzoate following step 3 and step 4 of intermediate BB-1. MS (ESI) m/z 279.0 [M+H]⁺.

### Intermediate BB-48. 2-(4-(bromomethyl)-2-methoxyphenyl)-1-ethyl-4-(trifluoromethyl)-1H-imidazole

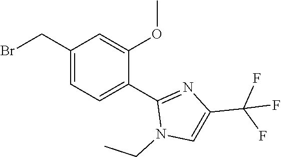 [SMILES: CCn1cc(C(F)(F)F)nc1-c1ccc(CBr)cc1OC]

2-(4-(bromomethyl)-2-methoxyphenyl)-1-ethyl-4-(trifluoromethyl)-1H-imidazole was prepared from 4-formyl-3-methoxybenzonitrile following Intermediate BB-4. MS (ESI) m/z 363.0, 365.0 [M+H]⁺.

### Intermediate BB-49. 2-(bromomethyl)-5-(1-ethyl-4-(trifluoromethyl)-1H-imidazol-2-yl)pyridine

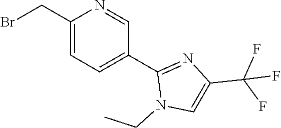 [SMILES: CCn1cc(C(F)(F)F)nc1-c1ccc(CBr)nc1]

2-(bromomethyl)-5-(1-ethyl-4-(trifluoromethyl)-1H-imidazol-2-yl)pyridine was prepared from methyl 5-formylpicolinate following Intermediate BB-1. MS (ESI) m/z 334.0, 336.0 [M+H]⁺.

### Intermediate BB-50. 2-(4-(bromomethyl)-2-(difluoromethoxy)phenyl)-1-ethyl-4-(trifluoromethyl)-1H-imidazole

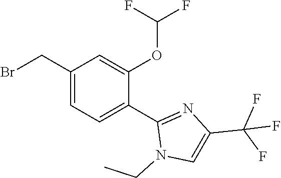 [SMILES: CCn1cc(C(F)(F)F)nc1-c1ccc(CBr)cc1OC(F)F]

2-(4-(bromomethyl)-2-(difluoromethoxy)phenyl)-1-ethyl-4-(trifluoromethyl)-1H-imidazole was prepared from 4-bromo-2-(difluoromethoxy)benzaldehyde following Intermediate BB-46. MS (ESI) m/z 399.0, 401.0 [M+H]⁺.

### Intermediate BB-51. 2-(4-(1-bromoethyl)phenyl)-1-methyl-4-(trifluoromethyl)-1H-imidazole

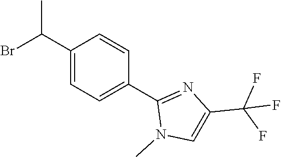 [SMILES: CC(Br)c1ccc(-c2nc(C(F)(F)F)cn2C)cc1]

2-(4-(1-bromoethyl)phenyl)-1-methyl-4-(trifluoromethyl)-1H-imidazole was prepared from 4-acetylbenzaldehyde following step 1 to step 4 of Intermediate BB-1. MS (ESI) m/z 333.1, 335.1 [M+H]⁺.

### Intermediate BB-52. 2-(4-(1-bromoethyl)-2-fluorophenyl)-1-ethyl-4-(trifluoromethyl)-1H-imidazole

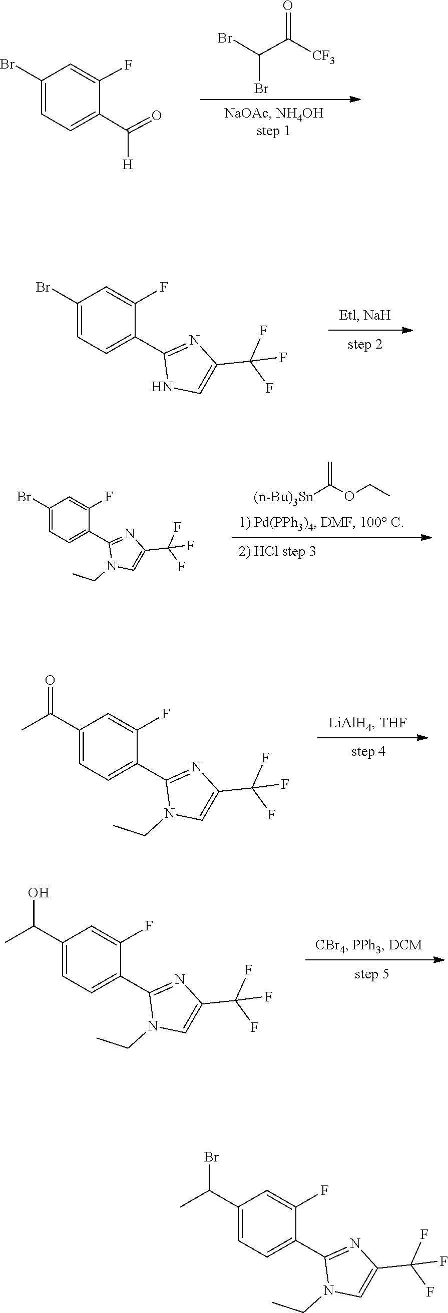

### Step 1 and step 2. 2-(4-bromo-2-fluorophenyl)-1-ethyl-4-(trifluoromethyl)-1H-imidazole

2-(4-bromo-2-fluorophenyl)-1-ethyl-4-(trifluoromethyl)-1H-imidazole was prepared from 4-bromo-2-fluorobenzaldehyde following step 1 and step 2 of intermediate BB-1. MS (ESI) m/z 337.0, 339.0 [M+H]⁺.

### Step 3. 1-(4-(1-ethyl-4-(trifluoromethyl)-1H-imidazol-2-yl)-3-fluorophenyl)ethan-1-one

A mixture of 2-(4-bromo-2-fluorophenyl)-1-ethyl-4-(trifluoromethyl)-1H-imidazole (2.0 g, 6.0 mmol), tributyl(1-ethoxyvinyl)stannane (2.6 g, 7.1 mmol) and Pd(PPh₃)₄ (347 mg, 0.3 mmol) in DMF (12 mL) was heated at 130° C. for 1 h in microwave reactor. The reaction mixture was diluted with H₂O (20 mL), extracted with EtOAc (20 mL×3). The combined organic layers were washed with water and brine, concentrated under vacuum. The resulting residue was stirred in a mixture of THF (5 mL) and aqueous HCl (2 M, 15 mL) for 30 min. The solvent was removed and the residue was purified on a Biotage Isolera One (C₁₈ column, eluting with 10% to 95% MeCN/H₂O) to afford 1-(4-(1-ethyl-4-(trifluoromethyl)-1H-imidazol-2-yl)-3-fluorophenyl)ethan-1-one (1.2 g). MS (ESI) m/z 301.1 [M+H]⁺.

### Step 4 and step 5. 2-(4-(1-bromoethyl)-2-fluorophenyl)-1-ethyl-4-(trifluoromethyl)-1H-imidazole

2-(4-(1-bromoethyl)-2-fluorophenyl)-1-ethyl-4-(trifluoromethyl)-1H-imidazole was synthesized from 1-(4-(1-ethyl-4-(trifluoromethyl)-1H-imidazol-2-yl)-3-fluorophenyl)ethan-1-one following step 3 and step 4 of Intermediate BB-1. MS (ESI) m/z 365.0, 367.0 [M+H]⁺.

### Intermediate BB-53. 2-(1-bromo-6-fluoro-2,3-dihydro-1H-inden-5-yl)-1-ethyl-4-(trifluoromethyl)-1H-imidazole

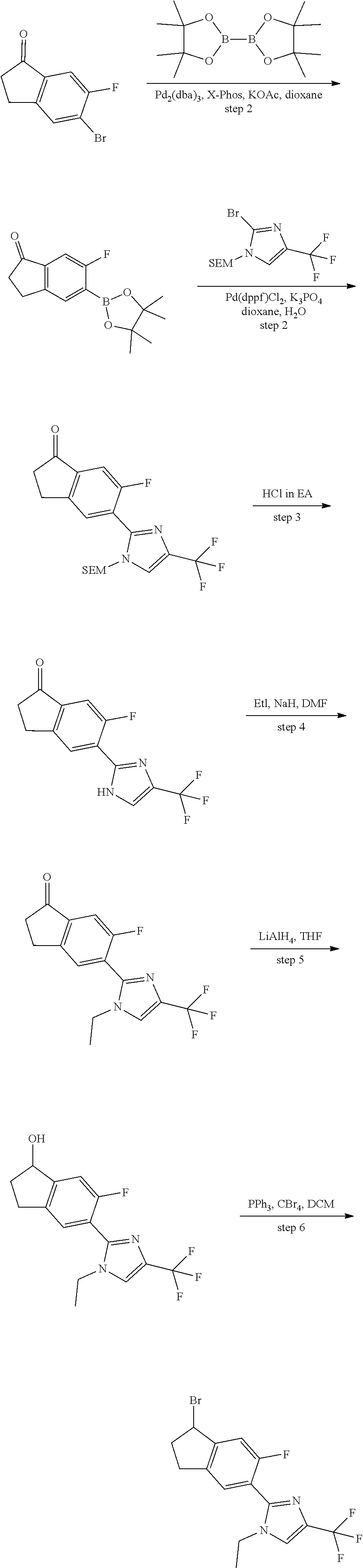

### Step 1. 6-fluoro-5-(4,4,5,5-tetramethyl-1,3,2-dioxaborolan-2-yl)-2,3-dihydro-1H-inden-1-one

A solution of 5-bromo-6-fluoro-2,3-dihydro-1H-inden-1-one (922 mg, 4.0 mmol), 4,4,4′,4′,5,5,5′,5′-octamethyl-2,2′-bi(1,3,2-dioxaborolane) (3.05 g, 12.0 mmol), Pd₂(dba)₃ (358 mg, 0.4 mmol), X-Phos (381 mg, 0.8 mmol) and KOAc (1.17 g, 12 mmol) in dioxane (20 mL) was heated at 90° C. for 4 h under N₂. The solution was filtered through celite and the filtrate concentrated under vacuum. The crude product was purified by silica gel chromatography (eluting with 1:3 EtOAc/PE) to afford 6-fluoro-5-(4,4,5,5-tetramethyl-1,3,2-dioxaborolan-2-yl)-2,3-dihydro-1H-inden-1-one (845 mg). MS (ESI) m/z 277.1 [M+H]⁺.

### Step 2 to step 6. 2-(1-bromo-6-fluoro-2,3-dihydro-1H-inden-5-yl)-1-ethyl-4-(trifluoromethyl)-1H-imidazole

2-(1-bromo-6-fluoro-2,3-dihydro-1H-inden-5-yl)-1-ethyl-4-(trifluoromethyl)-1H-imidazole was prepared from 6-fluoro-5-(4,4,5,5-tetramethyl-1,3,2-dioxaborolan-2-yl)-2,3-dihydro-1H-inden-1-one and 2-bromo-4-(trifluoromethyl)-1-((2-(trimethylsilyl)ethoxy)methyl)-1H-imidazole following step 3 to step 7 of Intermediate BB-39. MS (ESI) m/z 377.0 [M+H]⁺.

### Intermediate BB-54. 2-(4-(1-bromoethyl)phenyl)-1-isopropyl-4-(trifluoromethyl)-1H-imidazole

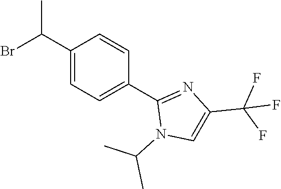 [SMILES: CC(Br)c1ccc(-c2nc(C(F)(F)F)cn2C(C)C)cc1]

2-(4-(1-bromoethyl)phenyl)-1-isopropyl-4-(trifluoromethyl)-1H-imidazole was prepared from 4-acetylbenzaldehyde following step 1 to step 4 of Intermediate BB-1. MS (ESI) m/z 361.0, 363.0 [M+H]⁺.

### Intermediate BB-55. 2-(4-(1-bromoethyl)-2-methoxyphenyl)-1-isopropyl-4-(trifluoromethyl)-1H-imidazole

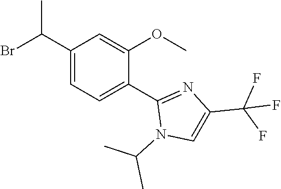 [SMILES: COc1cc(C(C)Br)ccc1-c1nc(C(F)(F)F)cn1C(C)C]

2-(4-(1-bromoethyl)-2-methoxyphenyl)-1-isopropyl-4-(trifluoromethyl)-1H-imidazole was synthesized following Intermediate BB-52. MS (ESI) m/z 391.1, 393.1 [M+H]⁺.

### Intermediate BB-56. 2-(4-(1-bromoethyl)-2-fluorophenyl)-1-isopropyl-4-(trifluoromethyl)-1H-imidazole

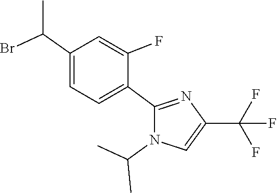 [SMILES: CC(Br)c1ccc(-c2nc(C(F)(F)F)cn2C(C)C)c(F)c1]

2-(4-(1-bromoethyl)-2-fluorophenyl)-1-isopropyl-4-(trifluoromethyl)-1H-imidazole was prepared following Intermediate BB-52. MS (ESI) m/z 379.0, 381.0 [M+H]⁺.

### Intermediate BB-57. 2-(4-(1-bromoethyl)-2-methoxyphenyl)-1-ethyl-4-(trifluoromethyl)-1H-imidazole

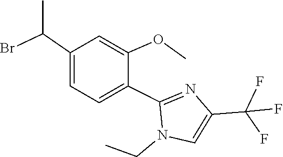 [SMILES: CCn1cc(C(F)(F)F)nc1-c1ccc(C(C)Br)cc1OC]

2-(4-(1-bromoethyl)-2-methoxyphenyl)-1-isopropyl-4-(trifluoromethyl)-1H-imidazole was synthesized following Intermediate BB-52. MS (ESI) m/z 377.0, 379.0 [M+H]⁺.

### Intermediate BB-58. 5-(1-bromoethyl)-2-(1-ethyl-4-(trifluoromethyl)-1H-imidazol-2-yl)pyridine

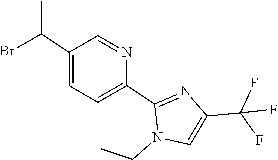 [SMILES: CCn1cc(C(F)(F)F)nc1-c1ccc(C(C)Br)cn1]

5-(1-bromoethyl)-2-(1-ethyl-4-(trifluoromethyl)-1H-imidazol-2-yl)pyridine was synthesized from 5-bromopicolinaldehyde following Intermediate BB-52. MS (ESI) m/z 348.0, 350.0 [M+H]⁺.

### Intermediate BB-59. 2-(4-(bromomethyl)-2-fluoro-6-methoxyphenyl)-1-isopropyl-4-(trifluoromethyl)-1H-imidazole

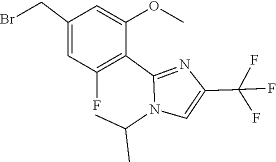 [SMILES: COc1cc(CBr)cc(F)c1-c1nc(C(F)(F)F)cn1C(C)C]

2-(4-(bromomethyl)-2-fluoro-6-methoxyphenyl)-1-isopropyl-4-(trifluoromethyl)-1H-imidazole was prepared following Intermediate BB-46. MS (ESI) m/z 395.0, 397.0 [M+H]⁺.

### Intermediate BB-60. 2-(4-(bromomethyl)phenyl)-1-(1-fluoropropan-2-yl)-4-(trifluoromethyl)-1H-imidazole

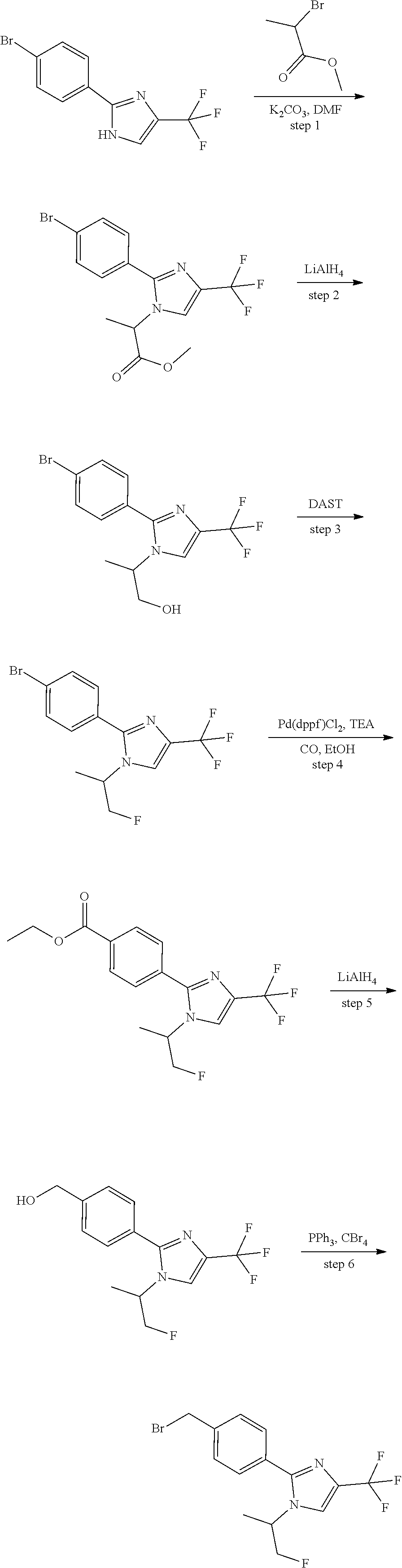

### Step 1. methyl 2-(2-(4-bromophenyl)-4-(trifluoromethyl)-1H-imidazol-1-yl)propanoate

A mixture of 2-(4-bromophenyl)-4-(trifluoromethyl)-1H-imidazole (3 g, 10.31 mmol), methyl 2-bromopropanoate (1.72 g, 10.31 mmol) and K₂CO₃ (2.85 g, 20.62 mmol) in dry DMF (10 mL) was stirred at room temperature for 2 h. The reaction mixture diluted with H₂O (20 mL), extracted with DCM (20 mL×3). The combined organic layers were concentrated and purified by column chromatography on silica gel (eluting with 4/1 PE/EtOAc) to give methyl 2-(2-(4-bromophenyl)-4-(trifluoromethyl)-1H-imidazol-1-yl)propanoate (2.9 g). MS (ESI) m/z 377.0, 379.0 [M+H]⁺.

### Step 2. 2-(2-(4-bromophenyl)-4-(trifluoromethyl)-1H-imidazol-1-yl)propan-1-ol

To a solution of methyl 2-(2-(4-bromophenyl)-4-(trifluoromethyl)-1H-imidazol-1-yl)propanoate (2.8 g ,7.43 mmol) in THF (10 mL) was added LiAlH₄ (338.7 mg, 8.91 mmol) slowly at 0° C. The reaction mixture was stirred at room temperature for 40 min then quenched with saturated Na₂CO₃ and quickly filtered through celite. The filtrate was concentrated to give crude 2-(2-(4-bromophenyl)-4-(trifluoromethyl)-1H-imidazol-1-yl)propan-1-ol (2.8 g) which was used directly in next step without further purification. MS (ESI) m/z 349.0, 351 [M+H]⁺.

### Step 3. 2-(4-bromophenyl)-1-(1-fluoropropan-2-yl)-4-(trifluoromethyl)-1H-imidazole

To a solution of 2-(2-(4-bromophenyl)-4-(trifluoromethyl)-1H-imidazol-1-yl)propan-1-ol (2.8 g, 8.0 mmol) in DCM (20 mL) was added DAST (40 mL). The mixture was stirred at room temperature for 16 h. Solvent was removed under vacuum. The resulting residue was purified by column chromatography on silica gel (eluting with 4/1 PE/EtOAc) to give 2-(4-bromophenyl)-1-(1-fluoropropan-2-yl)-4-(trifluoromethyl)-1H-imidazole (2.9 g). MS (ESI) m/z 351.0, 353.0 [M+H]⁺.

### Step 4 to Step 6. 2-(4-(bromomethyl)phenyl)-1-(1-fluoropropan-2-yl) (trifluoromethyl)-1H-imidazole

2-(4-(bromomethyl)phenyl)-1-(1-fluoropropan-2-yl)-4-(trifluoromethyl)-1H-imidazole was prepared from 2-(4-bromophenyl)-1-(1-fluoropropan-2-yl)-4-(trifluoromethyl)-1H-imidazole following step 4 to step 6 of Intermediate BB-11. MS (ESI) m/z 365.0, 367.0 [M+H]⁺.

### Intermediate BB-61. 2-(4-(1-bromoethyl)-2-fluorophenyl)-1-methyl-4-(trifluoromethyl)-1H-imidazole

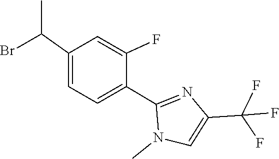 [SMILES: CC(Br)c1ccc(-c2nc(C(F)(F)F)cn2C)c(F)c1]

2-(4-(1-bromoethyl)-2-fluorophenyl)-1-methyl-4-(trifluoromethyl)-1H-imidazole was prepared following Intermediate BB-52. MS (ESI) m/z 351.0, 353.0 [M+H]⁺.

### Intermediate BB-62. 2-(4-(1-bromoethyl)phenyl)-1-ethyl-1H-imidazole-4-carbonitrile

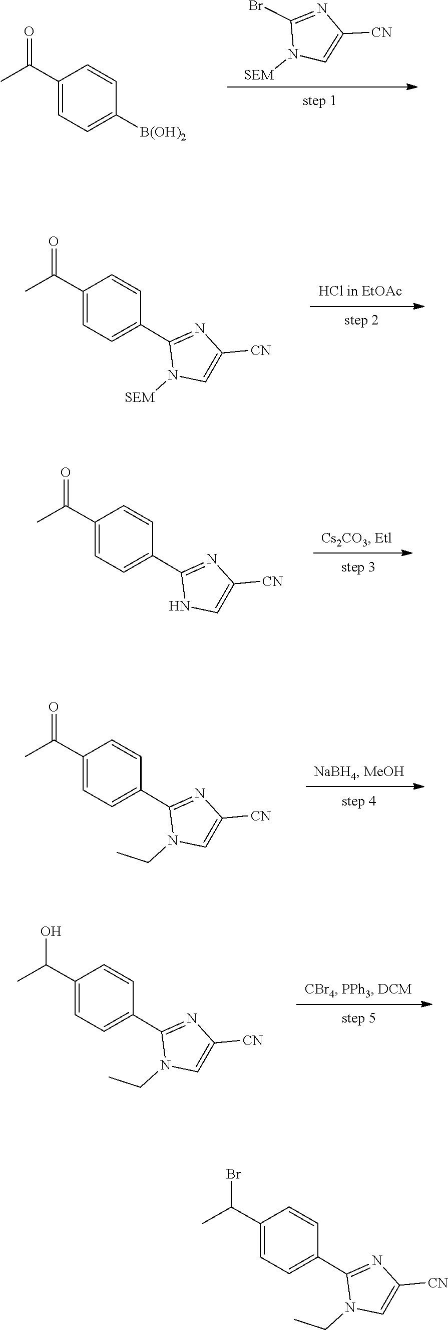

### Step 1 and step 2. 2-(4-acetylphenyl)-1H-imidazole-4-carbonitrile

2-(4-acetylphenyl)-1H-imidazole-4-carbonitrile was prepared from (4-acetylphenyl)boronic acid and 2-bromo-1-((2-(trimethylsilyl)ethoxy)methyl)-1H-imidazole-4-carbonitrile (prepared from 1H-imidazole-4-carbonitrile following step 1 and step 2 of Intermediate BB-39) following step 3 and step 4 of Intermediate BB-39. MS (ESI) m/z 212.1 [M+H]⁺.

### Step 3. 2-(4-acetylphenyl)-1-ethyl-1H-imidazole-4-carbonitrile

To a solution of 2-(4-acetylphenyl)-1H-imidazole-4-carbonitrile (2.2 g, 10.4 mmol) in MeCN (25 mL) was added EtI (1.8 g, 11.5 mmol) and Cs₂CO₃ (6.8 g, 20.9 mmol). The mixture was heated at 60° C. for 4 h then filtered through celite and concentrated under vacuum. The crude was purified by column chromatography on silica gel (eluting with 2/1 PE/EtOAc) to afford 2-(4-acetylphenyl)-1-ethyl-1H-imidazole-4-carbonitrile (1.0 g). MS (ESI) m/z 240.1 [M+H]⁺.

### Step 4. 1-ethyl-2-(4-(1-hydroxyethyl)phenyl)-1H-imidazole-4-carbonitrile

To a solution of 2-(4-acetylphenyl)-1-ethyl-1H-imidazole-4-carbonitrile (1.0, 4.2 mmol) in MeOH (15 mL) was added NaBH4 (318 mg, 8.4 mmol). The mixture was stirred at room temperature for 30 min. The mixture was concentrated under vacuum and purified by column chromatography on silica gel (eluting with 3/1 PE/EtOAc) to afford 1-ethyl-2-(4-(1-hydroxyethyl)phenyl)-1H-imidazole-4-carbonitrile (800 mg). MS (ESI) m/z 242.1 [M+H]⁺.

### Step 5. 2-(4-(1-bromoethyl)phenyl)-1-ethyl-1H-imidazole-4-carbonitrile

2-(4-(1-bromoethyl)phenyl)-1-ethyl-1H-imidazole-4-carbonitrile was prepared following step 4 of intermediate BB-1. MS (ESI) m/z 304.0, 306.0 [M+H]⁺.

### Intermediate BB-63. 2-(4-(1-bromoethyl)phenyl)-1-cyclopropyl-4-(trifluoromethyl)-1H-imidazole

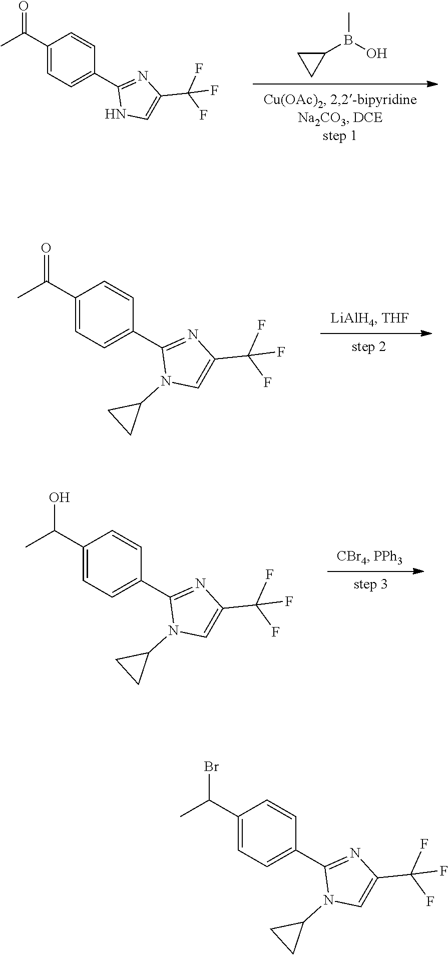

### Step 1. 1-(4-(1-cyclopropyl-4-(trifluoromethyl)-1H-imidazol-2-yl)phenyl)ethan-1-one

To a solution of 1-(4-(4-(trifluoromethyl)-1H-imidazol-2-yl)phenyl)ethan-1-one (2.0 g, 7.8 mmol), cyclopropylboronic acid (1.4 g 15.8 mmol) and Na₂CO₃ (1.7 g, 15.8 mmol) in 1,2-dichloroethane (10 mL) was added a solution of Cu(OAc)₂ (572.0 mg, 3.2 mmol) and 2,2′-bipyridine (1.2 g, 7.8 mmol) in 1,2-dichloroethane (20 mL). The resulting mixture was heated at 70° C. overnight under an atmosphere of O₂. The mixture was diluted with water (20 mL) and extracted with DCM (20 mL×2). The organic layers were washed with brine, dried over anhydrous Na₂SO₄, concentrated under vacuum and purified by column chromatography on silica gel (eluting with 4/1 PE/EtOAc) to afford 1-(4-(1-cyclopropyl-4-(trifluoromethyl)-1H-imidazol-2-yl)phenyl)ethan-1-one (1.8 g). MS (ESI) m/z 295.1[M+H]⁺.

### Step 2 and step 3. 2-(4-(1-bromoethyl)phenyl)-1-cyclopropyl-4-(trifluoromethyl)-1H-imidazole

2-(4-(1-bromoethyl)phenyl)-1-cyclopropyl-4-(trifluoromethyl)-1H-imidazole was prepared from 1-(4-(1-cyclopropyl-4-(trifluoromethyl)-1H-imidazol-2-yl)phenyl)ethan-1-one following step 3 and step 4 of intermediate BB-1. MS (ESI) m/z 347.0, 349.0 [M+H]⁺.

### Intermediate BB-64. 2-(4-(bromomethyl)-2-chloro-6-methoxyphenyl)-1-isopropyl-4-(trifluoromethyl)-1H-imidazole

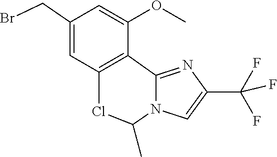

2-(4-(bromomethyl)-2-chloro-6-methoxyphenyl)-1-isopropyl-4-(trifluoromethyl)-1H-imidazole was prepared from 4-bromo-2-chloro-6-methoxybenzaldehyde following Intermediate BB-46. MS (ESI) m/z 411.0, 413.0 [M+H]⁺.

### Intermediate BB-65. 1-(4-(1-bromoethyl)-2-fluorophenyl)-5-methyl-3-(trifluoromethyl)-1H-pyrazole

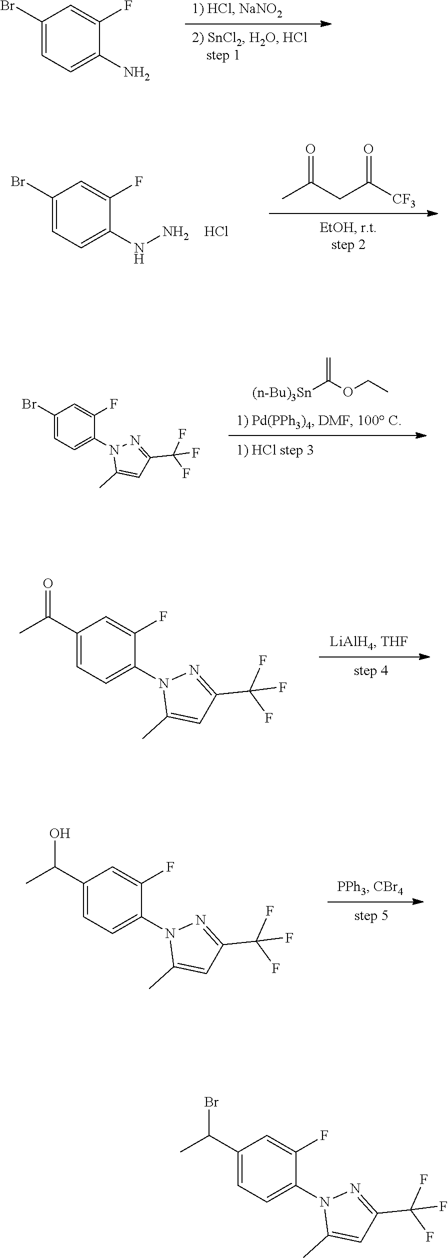

### Step 1 and step 2. 1-(4-bromo-2-fluorophenyl)-5-methyl-3-(trifluoromethyl)-1H-pyrazole

1-(4-bromo-2-fluorophenyl)-5-methyl-3-(trifluoromethyl)-1H-pyrazole was prepared from 4-bromo-2-fluoroaniline following step 1 and step 2 of Intermediate BB-8. ¹H NMR (400 MHz, Chloroform-d) δ 8.61 (s, 1H), 7.53 (dd, J=11.1, 1.3 Hz, 1H), 7.49-7.43 (m, 1H), 7.39 (t, J=7.8 Hz, 1H), 6.44 (s, 1H), 6.15 (q, J=7.2 Hz, 1H), 4.40 (td, J=7.3, 3.2 Hz, 2H), 3.96 (s, 3H), 3.09 (t, J=7.3 Hz, 2H), 2.23 (s, 3H), 2.00 (dd, J=13.1, 5.8 Hz, 4H), 1.24-1.18 (m, 2H), 0.96 (dd, J=8.0, 2.0 Hz, 2H).

### Step 3 to step 5. 1-(4-(1-bromoethyl)-2-fluorophenyl)-5-methyl-3-(trifluoromethyl)-1H-pyrazole

1-(4-(1-bromoethyl)-2-fluorophenyl)-5-methyl-3-(trifluoromethyl)-1H-pyrazole was prepared following step 3 to step 5 of Intermediate BB-52. MS (ESI) m/z 351.0, 353.0 [M+H]⁺.

### Intermediate BB-66. 2-(4-aminophenyl)-1-ethyl-1H-imidazole-4-carbonitrile

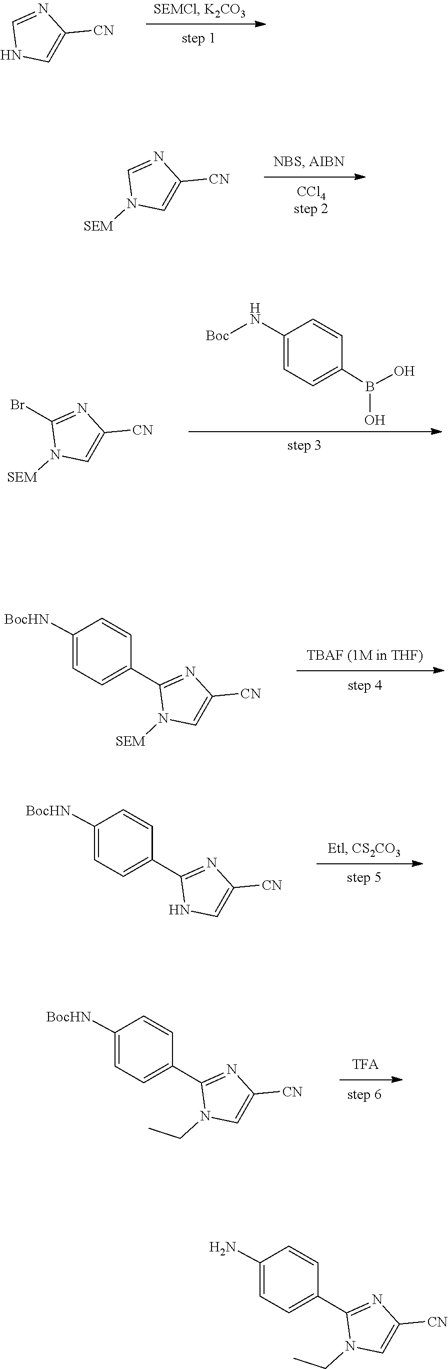

### Step 1 to step 3. tert-butyl-(4-(4-cyano-1-((2-(trimethylsilyl)ethoxy)methyl)-1H-imidazol-2-yl)phenyl)carbamate

tert-butyl-(4-(4-cyano-1-((2-(trimethylsilyl)ethoxy)methyl)-1H-imidazol yl)phenyl)carbamate was prepared from 1H-imidazole-4-carbonitrile following step 1 to step 3 of Intermediate BB-39.

### Step 4. tert-butyl-(4-(4-cyano-1H-imidazol-2-yl)phenyl)carbamate

A solution of tert-butyl-(4-(4-cyano-1-((2-(trimethylsilyl)ethoxy)methyl)-1H-imidazol-2-yl)phenyl)carbamate (1.2 g, 2.8 mmol) in TBAF (1 M in THF, 10 mL) was stirred at room temperature for 18 h. The solvent was removed under vacuum and the crude purified by column chromatography on silica gel (eluting with 2/1 PE/EtOAc) to give tert-butyl (4-(4-cyano-1H-imidazol-2-yl)phenyl)carbamate (370 mg). MS (ESI) m/z 285.1 [M+H]⁺.

### Step 5. tert-butyl-(4-(4-cyano-1-ethyl-1H-imidazol-2-yl)phenyl)carbamate

tert-butyl (4-(4-cyano-1-ethyl-1H-imidazol-2-yl)phenyl)carbamate was synthesized from tert-butyl-(4-(4-cyano-1H-imidazol-2-yl)phenyl)carbamate according to step 3 of Intermediate BB-62. MS (ESI) m/z 313.2 [M+H]⁺.

### Step 6. 2-(4-aminophenyl)-1-ethyl-1H-imidazole-4-carbonitrile

To a solution of tert-butyl-(4-(4-cyano-1-ethyl-1H-imidazol-2-yl)phenyl)carbamate (800 mg, 2.6 mmol) in DCM (10 mL) was added TFA (3 mL) at room temperature. The reaction solution was stirred at room temperature for 2 h. Solvent was removed under vacuum and the residue diluted with EtOAc, washed with 1M NaOH and brine. The organic phase was dried over Na₂SO₄ and concentrated under vacuum to give 2-(4-aminophenyl)-1-ethyl-1H-imidazole-4-carbonitrile (250 mg). MS (ESI) m/z 213.1 [M+H]⁺.

### Intermediate BB-67. 4-(1-(1-fluoropropan-2-yl)-4-(trifluoromethyl)-1H-imidazol-2-yl)aniline

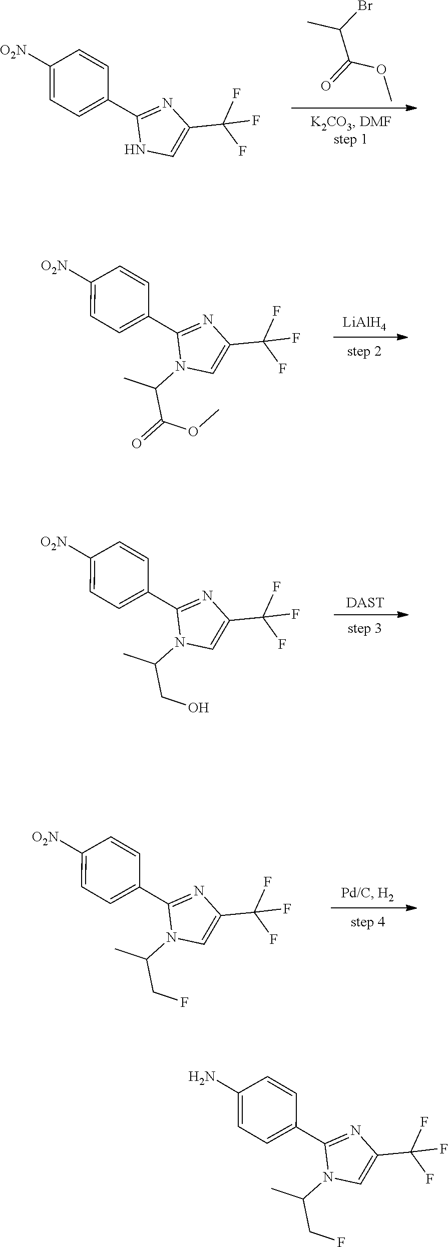

### Step 1 to step 3. 1-(1-fluoropropan-2-yl)-2-(4-nitrophenyl)-4-(trifluoromethyl)-1H-imidazole

1-(1-fluoropropan-2-yl)-2-(4-nitrophenyl)-4-(trifluoromethyl)-1H-imidazole was prepared from 2-(4-nitrophenyl)-4-(trifluoromethyl)-1H-imidazole following step 1 to step 3 of Intermediate BB-60. MS (ESI) m/z 318.1 [M+H]⁺.

### Step 4. 4-(1-(1-fluoropropan-2-yl)-4-(trifluoromethyl)-1H-imidazol-2-yl)aniline

4-(1-(1-fluoropropan-2-yl)-4-(trifluoromethyl)-1H-imidazol-2-yl)aniline was prepared according to step 3 of Intermediate BB-14. MS (ESI) m/z 288.1 [M+H]⁺.

### Intermediate BB-68. 4-(1-ethyl-4-(trifluoromethyl)-1H-imidazol-2-yl)phenol

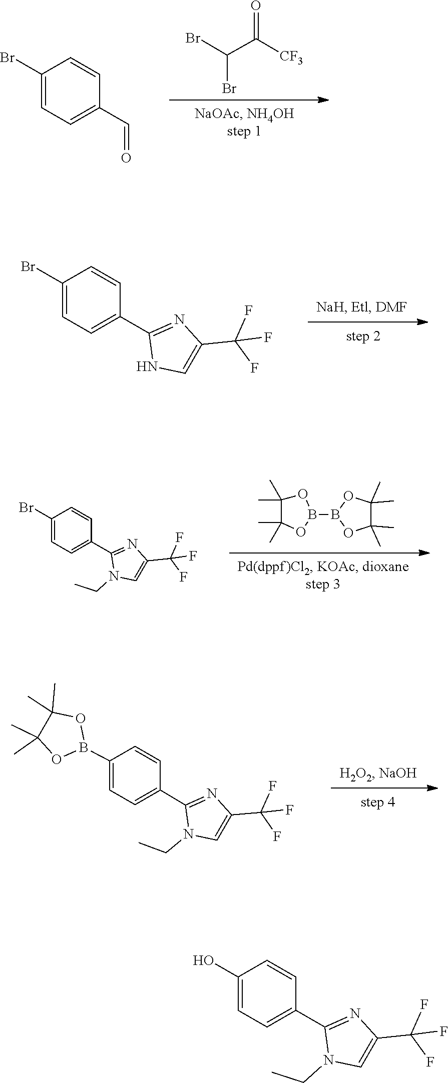

### Step 1 and step 2. 2-(4-bromophenyl)-1-ethyl-4-(trifluoromethyl)-1H-imidazole

2-(4-bromophenyl)-1-ethyl-4-(trifluoromethyl)-1H-imidazole was prepared from 4-bromobenzaldehyde following step 1 and step 2 of intermediate BB-1. MS (ESI) m/z 319.0, 401.0 [M+H]⁺.

### Step 3. 1-ethyl-2-(4-(4,4,5,5-tetramethyl-1,3,2-dioxaborolan-2-yl)phenyl) (trifluoromethyl)-1H-imidazole

1-ethyl-2-(4-(4,4,5,5-tetramethyl-1,3,2-dioxaborolan-2-yl)phenyl)-4-(trifluoromethyl)-1H-imidazole was prepared from 2-(4-bromophenyl)-1-ethyl-4-(trifluoromethyl)-1H-imidazole according to step 3 of intermediate 1. MS (ESI) m/z 367.2 [M+H]⁺.

### Step 4. 4-(1-ethyl-4-(trifluoromethyl)-1H-imidazol-2-yl)phenol

To a solution of 1-ethyl-2-(4-(4,4,5,5-tetramethyl-1,3,2-dioxaborolan-2-yl)phenyl)-4-(trifluoromethyl)-1H-imidazole (500 mg, 1.36 mmol) in THF (10 mL) was added 30% aqueous hydrogen peroxide (0.2 mL) and aqueous NaOH (2 M, 0.3 mL) at 0° C. The resulting mixture was stirred at room temperature for 2 h then extracted with DCM (50 mL×3). The combined organic phases were dried over anhydrous Na₂SO₄, concentrated and purified on a Biotage Isolera One (C₁₈ column, eluting with 10% to 95% MeCN/H₂O) to afford 4-(1-ethyl-4-(trifluoromethyl)-1H-imidazol-2-yl)phenol (278 mg). MS (ESI) m/z 257.1 [M+H]⁺.

### Intermediate BB-69. 4-(1-ethyl-4-(trifluoromethyl)-1H-imidazol-2-yl)-3-fluorophenol

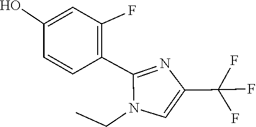 [SMILES: CCn1cc(C(F)(F)F)nc1-c1ccc(O)cc1F]

4-(1-ethyl-4-(trifluoromethyl)-1H-imidazol-2-yl)-3-fluorophenol was prepared from 2-fluoro-4-hydroxybenzaldehyde following Intermediate BB-68. MS (ESI) m/z 275.1 [M+H]⁺.

### Intermediate BB-70. 2-(4-(bromomethyl)-2-fluoro-3-methoxyphenyl)-1-isopropyl-4-(trifluoromethyl)-1H-imidazole

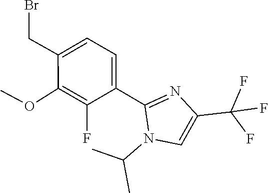 [SMILES: COc1c(CBr)ccc(-c2nc(C(F)(F)F)cn2C(C)C)c1F]

2-(4-(bromomethyl)-2-fluoro-3-methoxyphenyl)-1-isopropyl-4-(trifluoromethyl)-1H-imidazole was prepared from 4-bromo-2-fluoro-3-methoxybenzaldehyde following Intermediate BB-46. MS (ESI) m/z 395.0, 397.0 [M+H]⁺.

### Intermediate BB-71. 2-(4-(1-bromoethyl)-2-chlorophenyl)-1-ethyl-4-(trifluoromethyl)-1H-imidazole

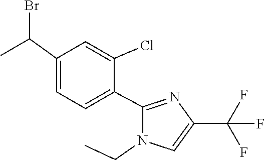 [SMILES: CCn1cc(C(F)(F)F)nc1-c1ccc(C(C)Br)cc1Cl]

2-(4-(1-bromoethyl)-2-chlorophenyl)-1-ethyl-4-(trifluoromethyl)-1H-imidazole was prepared from 4-bromo-2-chlorobenzaldehyde following Intermediate BB-52. MS (ESI) m/z 281.0, 283.0 [M+H]⁺.

### Intermediate BB-72. 3-fluoro-4-(3-methoxypyridin-2-yl)aniline

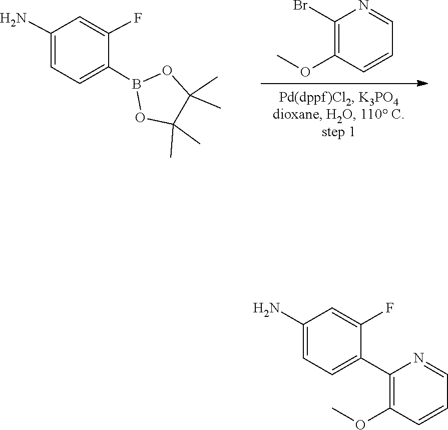

### Step 1. 3-fluoro-4-(3-methoxypyridin-2-yl)aniline

A mixture of 2-bromo-3-methoxypyridine (722 mg, 1.3 mmol), 3-fluoro-4-(4,4,5,5-tetramethyl-1,3,2-dioxaborolan-2-yl)aniline (700 mg, 1.0 mmol), Pd(dppf)Cl₂ (216 mg, 0.29 mmol) and K₃PO₄ (1.88 g, 8.86 mmol) in a mixture of dioxane (10 mL) and H₂O (1 mL) was heated at 110° C. for 1.5 h under N₂ in microwave reactor. After cooling to room temperature, the mixture was filtered through celite and the filtrate was concentrated under vacuum. The crude product was purified by silica gel chromatography (eluting with 1/2 EtOAc/PE) to afford 3-fluoro-4-(3-methoxypyridin-2-yl)aniline (350 mg). MS (ESI) m/z 219.1 [M+H]⁺.

### Intermediate BB-73. 3-chloro-4-(3-methoxypyridin-2-yl)aniline

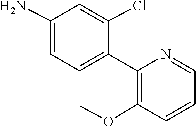 [SMILES: COc1cccnc1-c1ccc(N)cc1Cl]

3-chloro-4-(3-methoxypyridin-2-yl)aniline was prepared from 3-chloro-4-(4,4,5,5-tetramethyl-1,3,2-dioxaborolan-2-yl)aniline and 2-bromo-3-methoxypyridine following Intermediate BB-72. MS (ESI) m/z 235.0 [M+H]⁺.

### Intermediate BB-74. 5-amino-2-(1-ethyl-4-(trifluoromethyl)-1H-imidazol-2-yl)benzonitrile

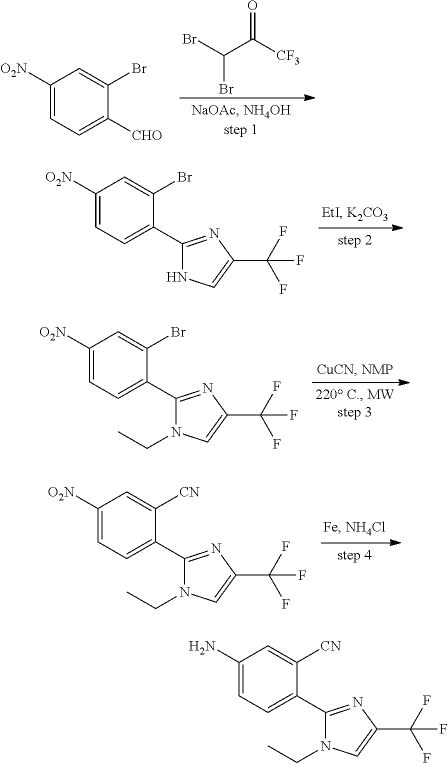

### Step 1. 2-(2-bromo-4-nitrophenyl)-4-(trifluoromethyl)-1H-imidazole

2-(2-bromo-4-nitrophenyl)-4-(trifluoromethyl)-1H-imidazole was synthesized from 2-bromo-4-nitrobenzaldehyde following step 1 of Intermediate BB-1. MS (ESI) m/z 335.9, 337.9 [M+H]⁺.

### Step 2. 2-(2-bromo-4-nitrophenyl)-1-ethyl-4-(trifluoromethyl)-1H-imidazole

To a solution of 2-(2-bromo-4-nitrophenyl)-4-(trifluoromethyl)-1H-imidazole (3.9 g, 11.6 mmol) in MeCN (100 mL) was added EtI (3.6 g, 23.2 mmol) and Cs₂CO₃ (7.6 g, 23.2 mmol). The mixture was stirred at room temperature for 18 h then was filtered through celite. The filtrate was concentrated under vacuum. The crude product was purified on a Biotage Isolera One (C₁₈ column, eluting with 10% to 95% MeCN/H₂O) to afford 2-(2-bromo-4-nitrophenyl)-1-ethyl-4-(trifluoromethyl)-1H-imidazole (1.24 g). MS (ESI) m/z 364.0, 366.0 [M+H]⁺.

### Step 3. 2-(1-ethyl-4-(trifluoromethyl)-1H-imidazol-2-yl)-5-nitrobenzonitrile

A mixture of 2-(2-bromo-4-nitrophenyl)-1-ethyl-4-(trifluoromethyl)-1H-imidazole (1.24 g, 3.4 mmol) and CuCN (922 mg, 10.2 mmol) in NMP (10 mL) was heated at 220° C. for 30 min in microwave reactor. The mixture was filtered through celite and the filtrate concentrated under vacuum. The crude was purified on a Biotage Isolera One (C₁₈ column, eluting with 10% to 95% MeCN/H₂O) to afford 2-(1-ethyl-4-(trifluoromethyl)-1H-imidazol-2-yl)-5-nitrobenzonitrile (400 mg). MS (ESI) m/z 311.1 [M+H]⁺.

### Step 4. 5-amino-2-(1-ethyl-4-(trifluoromethyl)-1H-imidazol-2-yl)benzonitrile

A mixture of 2-(1-ethyl-4-(trifluoromethyl)-1H-imidazol-2-yl)-5-nitrobenzonitrile (630 mg, 2.0 mmol), iron power (560 mg, 10 mmol) and NH₄Cl (162 mg, 3.0 mmol) in a mixture of EtOH (20 mL) and H₂O (4 mL) was heated at 85° C. for 3 h. The resulting mixture was filtered through celite and the filtrate concentrated under vacuum. The crude was purified by column chromatography on silica gel (eluting with 2/1 PE/EtOAc) to give 5-amino-2-(1-ethyl-4-(trifluoromethyl)-1H-imidazol-2-yl)benzonitrile (160 mg). MS (ESI) m/z 281.1 [M+H]⁺.

### Intermediate BB-75. 1-(4-(1-bromoethyl)phenyl)-5-(2,2-difluoroethoxy)-3-(trifluoromethyl)-1H-pyrazole

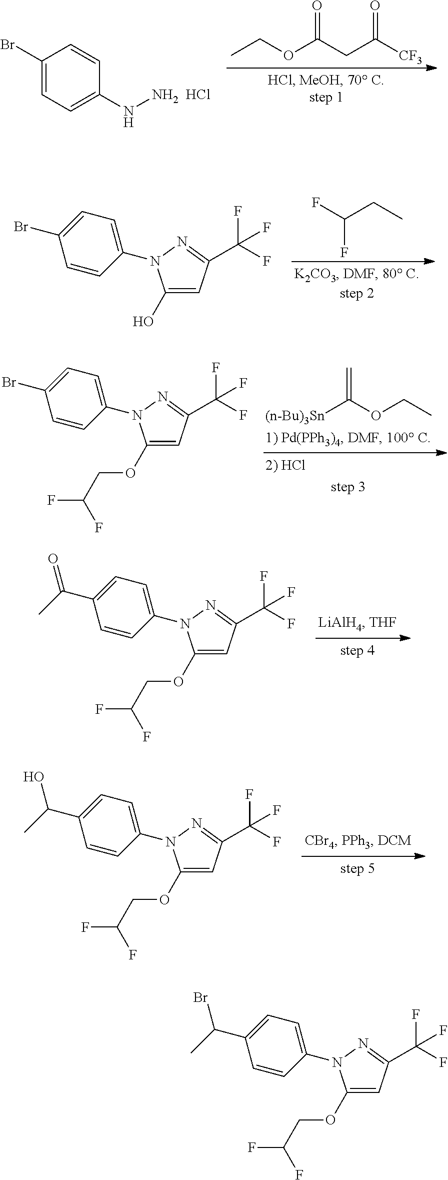

### Step 1. 1-(4-bromophenyl)-3-(trifluoromethyl)-1H-pyrazol-5-ol

1-(4-bromophenyl)-3-(trifluoromethyl)-1H-pyrazol-5-ol was prepared from (4-bromophenyl)hydrazine hydrochloride and ethyl 4,4,4-trifluoro-3-oxobutanoate following step 1 of intermediate BB-9. MS (ESI) m/z 307.0, 309.0 [M+H]⁺.

### Step 2. 1-(4-bromophenyl)-5-(2,2-difluoroethoxy)-3-(trifluoromethyl)-1H-pyrazole

A mixture of 1-(4-bromophenyl)-3-(trifluoromethyl)-1H-pyrazol-5-ol (600 mg, 1.95 mmol), 1,1-difluoro-2-iodoethane (563 mg, 2.93 mmol) and K₂CO₃ (810 mg, 5.86 mmol) in DMF (5 mL) was heated at 80° C. overnight. The resulting mixture was diluted with water (50 mL) and extracted with EtOAc (50 mL×2). The combine organic phases were washed with brine, dried over Na₂SO₄, concentrated under vacuum and purified by silica gel chromatography (eluting with 1/10 EtOAc/PE) to give 1-(4-bromophenyl)-5-(2,2-difluoroethoxy)-3-(trifluoromethyl)-1H-pyrazole (560 mg). MS (ESI) m/z 371.0, 373.0 [M+H]⁺.

### Step 3 to step 5. 1-(4-(1-bromoethyl)phenyl)-5-(2,2-difluoroethoxy)-3-(trifluoromethyl)-1H-pyrazole

1-(4-(1-bromoethyl)phenyl)-5-(2,2-difluoroethoxy)-3-(trifluoromethyl)-1H-pyrazole was prepared from 1-(4-bromophenyl)-5-(2,2-difluoroethoxy)-3-(trifluoromethyl)-1H-pyrazole following step 3 to step 5 of Intermediate BB-52. MS (ESI) m/z 399.0, 401.0 [M+H]⁺.

### Intermediate BB-76. 1-(4-(1-bromoethyl)phenyl)-5-methoxy-3-(trifluoromethyl)-1H-pyrazole

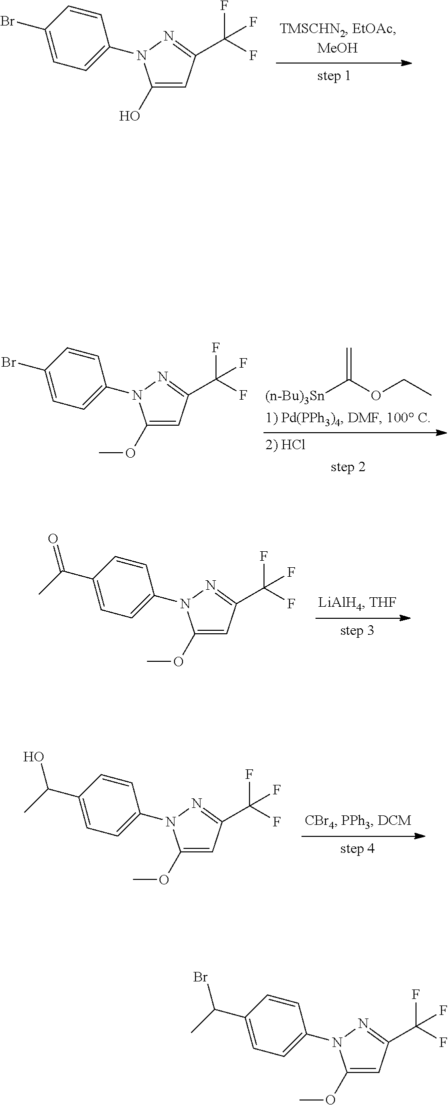

### Step 1. 1-(4-bromophenyl)-5-methoxy-3-(trifluoromethyl)-1H-pyrazole

1-(4-bromophenyl)-5-methoxy-3-(trifluoromethyl)-1H-pyrazole was prepared from 1-(4-bromophenyl)-3-(trifluoromethyl)-1H-pyrazol-5-ol (prepared in step 1 of Intermediate BB-75) following step 2 of Intermediate BB-22. MS (ESI) m/z 321.0, 323.0 [M+H]⁺.

### Step 2 to step 4. 1-(4-(1-bromoethyl)phenyl)-5-methoxy-3-(trifluoromethyl)-1H-pyrazole

1-(4-(1-bromoethyl)phenyl)-5-methoxy-3-(trifluoromethyl)-1H-pyrazole was synthesized from 1-(4-bromophenyl)-5-methoxy-3-(trifluoromethyl)-1H-pyrazole according to step 3 to step 5 of Intermediate BB-52. MS (ESI) m/z 349.0, 351.0 [M+H]⁺.

### Intermediate BB-77. 2-(4-(bromomethyl)-2-fluoro-3-methoxyphenyl)-1-ethyl-4-(trifluoromethyl)-1H-imidazole

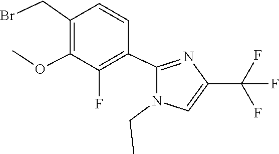 [SMILES: CCn1cc(C(F)(F)F)nc1-c1ccc(CBr)c(OC)c1F]

2-(4-(bromomethyl)-2-fluoro-3-methoxyphenyl)-1-ethyl-4-(trifluoromethyl)-1H-imidazole was prepared from 4-bromo-2-fluoro-3-methoxybenzaldehyde following Intermediate BB-46. MS (ESI) m/z 381.0, 383.0 [M+H]⁺.

### Intermediate BB-78. 2-(4-(bromomethyl)-2-fluoro-3-methoxyphenyl)-1-isopropyl-4-(trifluoromethyl)-1H-imidazole

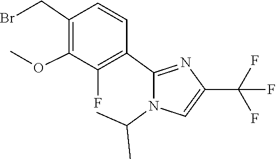 [SMILES: COc1c(CBr)ccc(-c2nc(C(F)(F)F)cn2C(C)C)c1F]

2-(4-(bromomethyl)-2-fluoro-3-methoxyphenyl)-1-isopropyl-4-(trifluoromethyl)-1H-imidazole was prepared from 4-bromo-2-fluoro-3-methoxybenzaldehyde following Intermediate BB-46. MS (ESI) m/z 395.0, 397.0 [M+H]⁺.

### Intermediate BB-79. 2-(4-(1-bromoethyl)-2-fluoro-3-methoxyphenyl)-1-isopropyl-4-(trifluoromethyl)-1H-imidazole

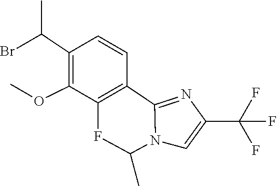

2-(4-(1-bromoethyl)-2-fluoro-3-methoxyphenyl)-1-isopropyl-4-(trifluoromethyl)-1H-imidazole was prepared from 4-bromo-2-fluoro-3-methoxybenzaldehyde following Intermediate BB-52. MS (ESI) m/z 409.1, 411.1 [M+H]⁺.

### Intermediate BB-80. 2-(4-(1-bromoethyl)phenyl)-3-isopropoxypyridine

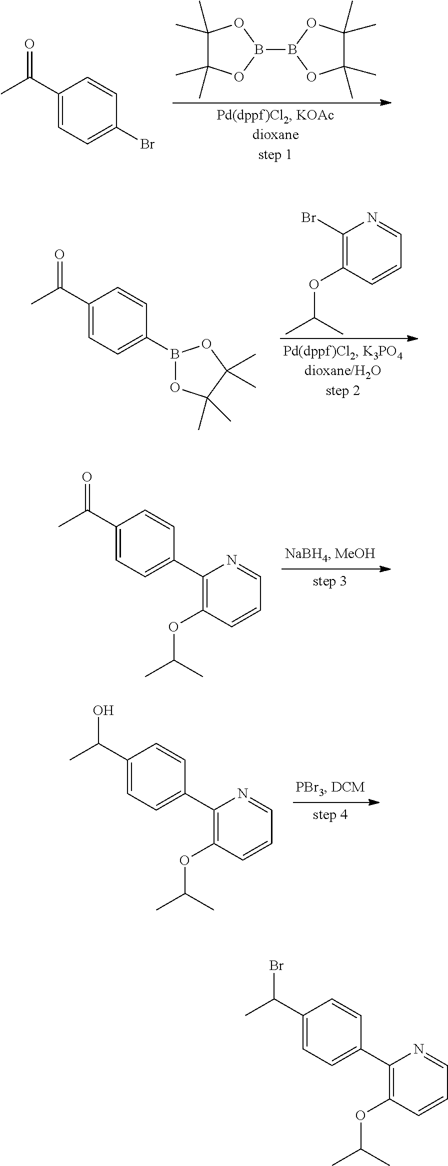

### Step 1. 1-(4-(4,4,5,5-tetramethyl-1,3,2-dioxaborolan-2-yl)phenyl)ethan-1-one

A mixture of 1-(4-bromophenyl)ethan-1-one (5 g, 25.1 mmol), 4,4,4′,4′,5,5,5′,5′-octamethyl-2,2′-bi(1,3,2-dioxaborolane) (12.8 g, 50.2 mmol), KOAc (4.9 g, 50.2 mmol) and Pd(dppf)Cl₂ (290 mg, 1.3 mmol) in dioxane (50 mL) was heated under an atmosphere of N₂ at 90° C. for 3 h. The resulting mixture was filtered through celite, concentrated and purified by silica gel chromatography (eluting with 1/10 EtOAc/PE) to afford 1-(4-(4,4,5,5-tetramethyl-1,3,2-dioxaborolan-2-yl)phenyl)ethan-1-one (5.1 g). MS (ESI) m/z 247.1 [M+H]⁺.

### Step 2. 1-(4-(3-isopropoxypyridin-2-yl)phenyl)ethan-1-one

1-(4-(3-isopropoxypyridin-2-yl)phenyl)ethan-1-one was synthesized from 1-(4-(4,4,5,5-tetramethyl-1,3,2-dioxaborolan-2-yl)phenyl)ethan-1-one according to Intermediate BB-72. MS (ESI) m/z 256.1 [M+H]⁺.

### Step 3. 1-(4-(3-isopropoxypyridin-2-yl)phenyl)ethan-1-ol

1-(4-(3-isopropoxypyridin-2-yl)phenyl)ethan-1-ol was prepared from 1-(4-(3-isopropoxypyridin-2-yl)phenyl)ethan-1-one following step 4 of Intermediate BB-62. MS (ESI) m/z 258.2 [M+H]⁺.

### Step 4. 2-(4-(1-bromoethyl)phenyl)-3-isopropoxypyridine

To a solution of 1-(4-(3-isopropoxypyridin-2-yl)phenyl)ethan-1-ol (123 mg, 0.48 mmol) in dry DCM (5 mL) was added PBr₃ (260 mg, 0.96 mmol) at 0° C. The resulting mixture was stirred at same temperature for 2 h then quenched with saturated aqueous NaHCO₃ (10 mL) and extracted with DCM (10 mL×3). The combined organic layers were dried over anhydrous Na₂SO₄, concentrated under vacuum and purified by silica gel chromatography (eluting with 1/5 EtOAc/PE) to afford 2-(4-(1-bromoethyl)phenyl)-3-isopropoxypyridine (86 mg). MS (ESI) m/z 320.0, 322.0 [M+H]⁺.

### Intermediate BB-81. 2-(4-(1-bromoethyl)phenyl)-N,N-dimethylpyridin-3-amine

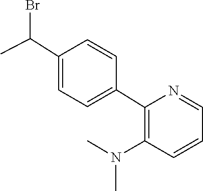 [SMILES: CC(Br)c1ccc(-c2ncccc2N(C)C)cc1]

2-(4-(1-bromoethyl)phenyl)-N,N-dimethylpyridin-3-amine was prepared according to Intermediate BB-80. MS (ESI) m/z 305.1, 307.1 [M+H]⁺.

### Example 1. 2-(4-cyclopropyl-6-methoxypyrimidin-5-yl)-4-(4-(1-methyl-4-(trifluoromethyl)-1H-imidazol-2-yl)benzyl)-6,7-dihydropyrazolo[1,5-α]pyrimidin-5(4H)-one

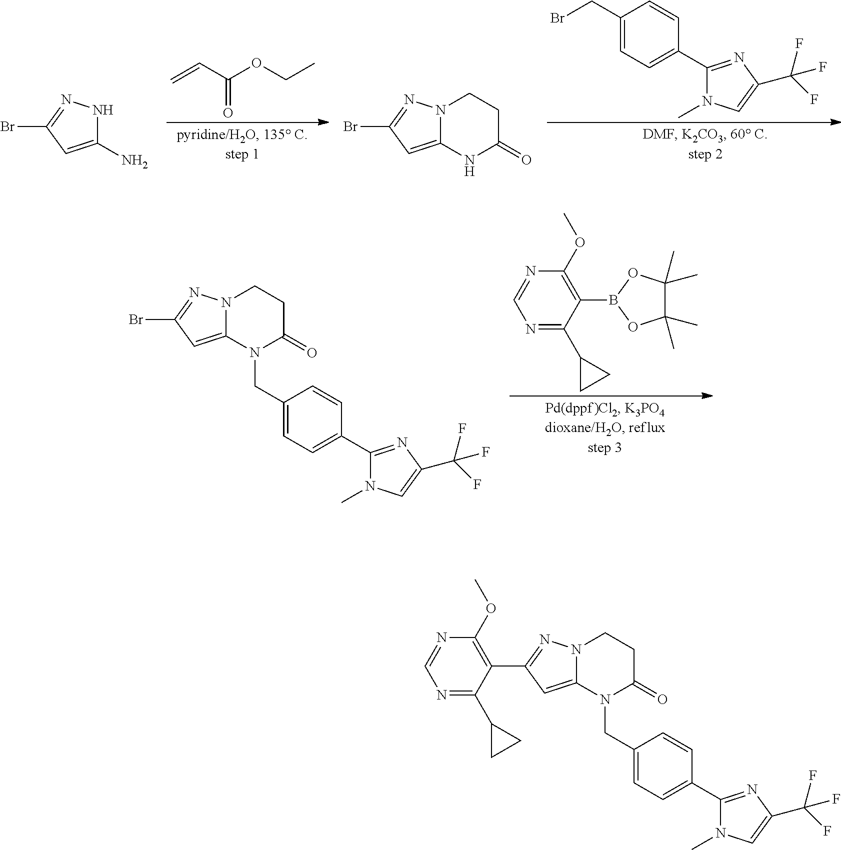

### Step 1. 2-bromo-6,7-dihydropyrazolo[1,5-α]pyrimidin-5(4H)-one

A mixture of 3-bromo-1H-pyrazol-5-amine (600 mg, 3.704 mmol) and ethyl acrylate (0.592 mL, 5.556 mmol) in pyridine (10 mL) and H₂O (1 mL) and was heated at 135° C. for 24 h in a sealed tube. After cooling to ambient temperature, the mixture was poured into EtOAc (15 mL) and the precipitate that formed was collected by filtration to afford 2-bromo-6,7-dihydropyrazolo[1,5-α]pyrimidin-5(4H)-one (270 mg). MS (ESI) m/z 216.0, 218.0 [M+H]⁺.

### Step 2. 2-bromo-4-(4-(1-methyl-4-(trifluoromethyl)-1H-imidazol-2-yl)benzyl)-6,7-dihydropyrazolo[1,5-α]pyrimidin-5(4H)-one

To a solution of 2-bromo-6,7-dihydropyrazolo[1,5-α]pyrimidin-5(4H)-one (150 mg, 0.694 mmol) in DMF (5 mL) was added 2-(4-(bromomethyl)phenyl)-1-methyl-4-(trifluoromethyl)-1H-imidazole (221 mg, 0.694 mmol) and K₂CO₃ (192 mg, 1.39 mmol). The resulting mixture was heated at 60° C. for 2 h. After cooling to ambient temperature, the mixture was poured into water (30 mL) and extracted with EtOAc (3×30 mL). The organic layers were combined, dried over anhydrous Na₂SO₄ and concentrated under vacuum. The residue was purified by silica gel chromatography (eluting with 1/2 EtOAc/PE) to afford 2-bromo-4-(4-(1-methyl-4-(trifluoromethyl)-1H-imidazol-2-yl)benzyl)-6,7-dihydropyrazolo[1,5-α]pyrimidin-5(4H)-one (302 mg). MS (ESI) m/z 454.0, 456.0 [M+H]⁺.

### Step 3. 2-(4-cyclopropyl-6-methoxypyrimidin-5-yl)-4-(4-(1-methyl-4-(trifluoromethyl)-1H-imidazol-2-yl)benzyl)-6,7-dihydropyrazolo[1,5-α]pyrimidin-5(4H)-one

To a solution of 2-bromo-4-(4-(1-methyl-4-(trifluoromethyl)-1H-imidazol-2-yl)benzyl)-6,7-dihydropyrazolo[1,5-α]pyrimidin-5(4H)-one (300 mg, 0.661 mmol) in a mixture of dioxane (10 mL) and H₂O (1 mL) was added 4-cyclopropyl-6-methoxy-5-(4,4,5,5-tetramethyl-1,3,2-dioxaborolan-2-yl)pyrimidine (183 mg, 0.663 mmol), Pd(dppf)Cl₂ (48 mg, 0.066 mmol) and K₃PO₄ (281 mg, 1.32 mmol). The mixture was heated at reflux for 16 h under N₂. After cooling to ambient temperature, the mixture was filtered through celite and the filtrate was concentrated under vacuum. The residue was purified by prep-HPLC (condition 1) to afford Example 1 2-(4-cyclopropyl-6-methoxypyrimidin-5-yl)-4-(4-(1-methyl-4-(trifluoromethyl)-1H-imidazol-2-yl)benzyl)-6,7-dihydropyrazolo[1,5-α]pyrimidin-5(4H)-one (110 mg).

Table 1. The compounds listed in Table 1 were synthesized according to Example 1 using the appropriate commercially available reagents and/or intermediates described above. Enantiomers were not separated.

TABLE 1

| Example | | | |
|---|---|---|---|
| number | Structure | ¹H NMR (δ ppm) | MS (ESI) |
| 1 | 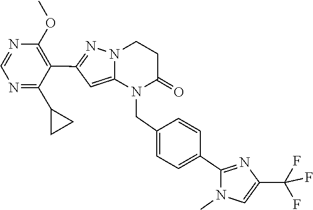 [SMILES: COc1ncnc(C2CC2)c1-c1cc2n(n1)CCC(=O)N2Cc1ccc(-c2nc(C(F)(F)F)cn2C)cc1] | ¹H NMR (400 MHz, Chloroform-d) δ 8.57 (s, 1H), 7.69-7.52 (m, 3H), 7.45 (d, J = 8.0 Hz, 2H), 7.30 (s, 1H), 5.86 (s, 1H), 5.04 (s, 2H), 4.45 (t, J = 7.2 Hz, 2H), 3.92 (s, 3H), 3.76 (s, 3H), 3.11 (t, J = 7.2 Hz, 2H), 2.18-2.22 (m, 1H), 1.19-1.20 (m, 2H), 0.94- 0.92 (m, 2H). | m/z: 523.9 [M + H]⁺ |
| | 2-(4-cyclopropyl-6- | | |
| | methoxypyrimidin-5-yl)-4-(4- | | |
| | (1-methyl-4-(trifluoromethyl)- | | |
| | 1H-imidazol-2-yl)benzyl)-6,7- | | |
| | dihydropyrazolo[1,5- | | |
| | a]pyrimidin-5(4H)-one | | |
| 2 | 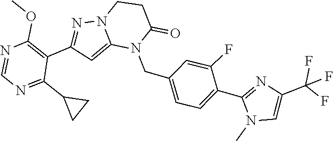 2-(4-cyclopropyl-6- methoxypyrimidin-5-yl)-4-(3- | ¹H NMR (400 MHz, Chloroform-d) δ 8.56 (s, 1H), 7.59 (t, J = 7.6 Hz, 1H), 7.35 (d, J = 0.8 Hz, 1H), 7.24 (d, J = 1.2 Hz, 1H), 7.17 (d, J = 10.4 Hz, 1H), 5.85 (s, 1H), 5.03 (s, 2H), 4.46 (t, J = 7.2 Hz, 2H), 3.92 (s, 3H), 3.64 (s, 3H), 3.12 (t, J = 6.8 Hz, 2H), 2.21-2.18 (m, 1H), 1.17-1.19 (m, 2H), 0.95-0.92 (m, 2H). | m/z: 542.2 [M + H]⁺ |
| | fluoro-4-(1-methyl-4- | | |
| | (trifluoromethyl)-1H- | | |
| | imidazol-2-yl)benzyl)-6,7- | | |
| | dihydropyrazolo[1,5- | | |
| | a]pyrimidin-5(4H)-one | | |
| 3 | 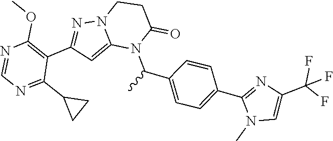 2-(4-cyclopropyl-6- methoxypyrimidin-5-yl)-4-(1- | ¹H NMR (400 MHz, Methanol-d₄) δ 8.45 (s, 1H), 7.69 (s, 1H), 7.68 (d, J = 8.4 Hz, 2H), 7.59 (d, J = 8.4 Hz, 2H), 6.27-6.21 (m, 1H), 5.59 (s, 1H), 4.44 (t, J = 6.8 Hz, 2H), 3.82 (s, 3H), 3.78 (s, 3H), 3.25-3.02 (m, 2H), 2.06- 1.97 (m, 1H), 1.86 (d, J = 7.2 Hz, 3H), 1.03-0.98 (m, 2H), 0.91-0.79 (m, 2H). | m/z: 538.3 [M + H]⁺ |
| | (4-(1-methyl-4- | | |
| | (trifluoromethyl)-1H- | | |
| | imidazol-2-yl)phenyl)ethyl)- | | |
| | 6,7-dihy dropyrazolo[1,5- | | |
| | a]pyrimidin-5(4H)-one | | |
| 4 | 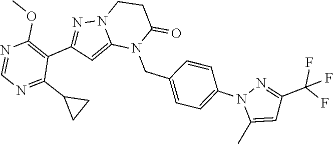 [SMILES: COc1ncnc(C2CC2)c1-c1cc2n(n1)CCC(=O)N2Cc1ccc(-n2nc(C(F)(F)F)cc2C)cc1] 2-(4-cyclopropyl-6- | ¹H NMR (400 MHz, Methanol-d₄) δ 8.58 (s, 1H), 7.57 (d, J = 8.4 Hz, 2H), 7.49 (d, J = 8.4 Hz, 2H), 6.57 (s, 1H), 6.04 (s, 1H), 5.12 (s, 2H), 4.45 (t, J = 6.8 Hz, 2H), 3.93 (s, 3H), 3.14 (t, J = 6.8 Hz, 2H), 2.33 (s, 3H), 2.26- 2.17 (m, 1H), 1.13-1.10 (m, 2H), 1.02-0.96 (m, 2H). | m/z: 524.3 [M + H]⁺ |
| | methoxypyrimidin-5-yl)-4-(4- | | |
| | (5-methyl-3-(trifluoromethyl)- | | |
| | 1H-pyrazol-1-yl)benzyl)-6,7- | | |
| | dihydropyrazolo[1,5- | | |
| | a]pyrimidin-5(4H)-one | | |
| 5 | 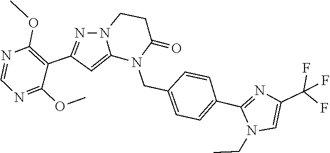 [SMILES: CCn1cc(C(F)(F)F)nc1-c1ccc(CN2C(=O)CCn3nc(-c4c(OC)ncnc4OC)cc32)cc1] | ¹H NMR (400 MHz, Chloroform-d) δ 8.42 (s, 1H), 7.56 (d, J = 8.0 Hz, 2H), 7.44 (d, J = 8.0 Hz, 2H), 7.36 (s, 1H), 5.95 (s, 1H), 5.03 (s, 2H), 4.47 (t, J = 6.8 Hz, 2H), 4.06 (q, J = 8.0 Hz, 2H), 3.96 (s, 6H), 3.09 (q, J = 6.8 Hz, 2H), 1.43 (t, J = 7.2 Hz, 3H). | m/z: 528.3 [M + H]⁺ |
| | 2-(4, 6-dimethoxypyrimidin-5- | | |
| | yl)-4-(4-(1-ethyl-4- | | |
| | (trifluoromethyl)-1H- | | |
| | imidazol-2-yl)benzyl)-6,7- | | |
| | dihydropyrazolo[1,5- | | |
| | a]pyrimidin-5(4H)-one | | |
| 6 | 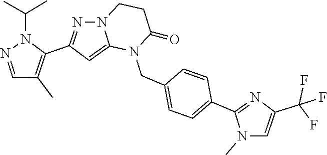 [SMILES: Cc1cnn(C(C)C)c1-c1cc2n(n1)CCC(=O)N2Cc1ccc(-c2nc(C(F)(F)F)cn2C)cc1] | ¹H NMR (400 MHz, Chloroform-d) δ 7.61 (t, J = 5.2 Hz, 2H), 7.43 (d, J = 2.8 Hz, 2H), 7.39 (s, 1H), 7.30 (s, 1H), 5.78 (s, 1H), 5.04 (s, 2H), 4.92 (s, 1H), 4.44 (s, 2H), 3.74 (s, 3H), 3.11 (s, 2H), 2.03 (s, 3H), 1.45 (s, 6H). | m/z: 498.2 [M + H]⁺ |
| | 2-(1-isopropyl-4-methyl-1H- | | |
| | pyrazol-5-yl)-4-(4-(1-methyl- | | |
| | 4-(trifluoromethyl)-1H- | | |
| | imidazol-2-yl)benzyl)-6,7- | | |
| | dihydropyrazolo[1,5- | | |
| | a]pyrimidin-5(4H)-one | | |
| 7 | 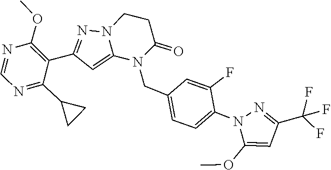 [SMILES: COc1ncnc(C2CC2)c1-c1cc2n(n1)CCC(=O)N2Cc1ccc(-n2nc(C(F)(F)F)cc2OC)c(F)c1] | ¹H NMR (400 MHz, Chloroform-d) δ 8.75 (s, 1H), 7.46 (t, J = 7.6 Hz, 1H), 7.24- 7.19 (m, 2H), 5.93 (s, 1H), 5.86 (s, 1H), 5.03 (s, 2H), 4.48 (t, J = 6.4 Hz, 2H), 3.99 (s, 3H), 3.94 (s, 3H), 3.13 (t, J = 7.2 Hz, 2H), 2.26 (t, J = 4.4 Hz, 1H), 1.34 (t, J = 7.6 Hz, 2H), 1.09-1.07 (m, 2H). | m/z: 558.3 [M + H]⁺ |
| | 2-(4-cyclopropyl-6- | | |
| | methoxypyrimidin-5-yl)-4-(3- | | |
| | fluoro-4-(5-methoxy-3- | | |
| | (trifluoromethyl)-1H-pyrazol- | | |
| | 1-yl)benzyl)-6,7- | | |
| | dihydropyrazolo[1,5- | | |
| | a]pyrimidin-5(4H)-one | | |
| 8 | 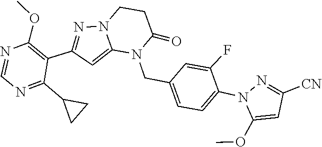 [SMILES: COc1ncnc(C2CC2)c1-c1cc2n(n1)CCC(=O)N2Cc1ccc(-n2nc(C#N)cc2OC)c(F)c1] (4-((2-(4-cyclopropyl-6- | ¹H NMR (400 MHz, Chloroform-d) δ 8.56 (s, 1H), 7.42 (t, J = 7.2 Hz, 1H), 7.24 (d, J = 7.2 Hz, 2H), 6.03 (s, 1H), 5.86 (s, 1H), 5.02 (s, 2H), 4.46 (t, J = 6.8 Hz, 2H), 3.93 (s, 3H), 3.92 (s, 3H), 3.12 (t, J = 6.8 Hz, 2H), 2.21 (s, 1H), 1.18 (s, 2H), 0.94 (d, J = 6.0 Hz, 2H). | m/z: 515.3 [M + H]⁺ |
| | methoxypyrimidin-5-yl)-5- | | |
| | oxo-6,7-dihydropyrazolo[1,5- | | |
| | a]pyrimidin-4(5H)- | | |
| | yl)methyl)-2-fluorophenyl)-5- | | |
| | methoxy-1H-pyrazole-3- | | |
| | carbonitrile | | |
| 9 | 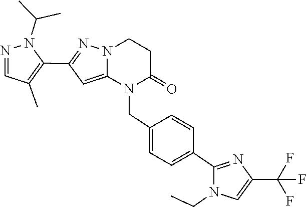 [SMILES: CCn1cc(C(F)(F)F)nc1-c1ccc(CN2C(=O)CCn3nc(-c4c(C)cnn4C(C)C)cc32)cc1] | ¹H NMR (400 MHz, Chloroform-d) δ 7.56 (d, J = 8.4 Hz, 2H), 7.42 (d, J = 8.0 Hz, 2H), 7.35 (s, 2H), 5.77 (s, 1H), 5.04 (s, 2H), 4.92-4.85 (m, 1H), 4.43 (t, J = 7.2 Hz, 2H), 4.04 (q, J = 7.2 Hz, 2H), 3.10 (t, J = 12 Hz, 2H), 2.01 (s, 3H), 1.43-1.39 (m, 9H). | m/z: 512.2 [M + H]⁺ |
| | 4-(4-(1-ethyl-4- | | |
| | (trifluoromethyl)-1H- | | |
| | imidazol-2-yl)benzyl)-2-(1- | | |
| | isopropyl-4-methyl-1H- | | |
| | pyrazol-5-yl)-6,7- | | |
| | dihydropyrazolo[1,5- | | |
| | a]pyrimidin-5(4H)-one | | |
| 10 | 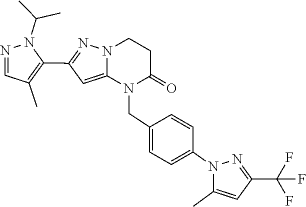 [SMILES: Cc1cnn(C(C)C)c1-c1cc2n(n1)CCC(=O)N2Cc1ccc(-n2nc(C(F)(F)F)cc2C)cc1] | ¹H NMR (400 MHz, Methanol-d₄) δ 7.58 (d, J = 8.4 Hz, 2H), 7.50 (d, J = 8.8 Hz, 2H), 7.40 (s, 1H), 6.57 (s, 1H), 6.02 (s, 1H), 5.15 (s, 2H), 4.47 (t, J = 7.2 Hz, 2H), 3.15 (t, J = 6.8 Hz, 2H), 2.32 (s, 3H), 2.01 (s, 3H), 1.38 (d, J = 6.4 Hz, 6H). | m/z: 498.2 [M + H]⁺ |
| | 2-(1-isopropyl-4-methyl-1H- | | |
| | pyrazol-5-yl)-4-(4-(5-methyl- | | |
| | 3-(trifluoromethyl)-1H- | | |
| | pyrazol-1-yl)benzyl)-6,7- | | |
| | dihydropyrazolo[1, 5- | | |
| | a]pyrimidin-5(4H)-one | | |
| 11 | 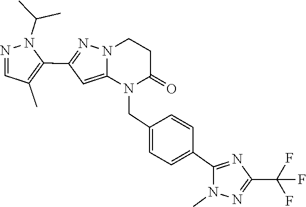 [SMILES: Cc1cnn(C(C)C)c1-c1cc2n(n1)CCC(=O)N2Cc1ccc(-c2nc(C(F)(F)F)nn2C)cc1] | ¹H NMR (400 MHz, Chloroform-d) δ 7.69 (d, J = 8.4 Hz, 2H), 7.49 (d, J = 8.4 Hz, 2H), 7.36 (s, 1H), 5.76 (s, 1H), 5.06 (s, 2H), 4.91-4.86 (m, 1H), 4.46 (t, J = 7.2 Hz, 2H) , 4.03 (s, 3H), 3.14 (t, J = 7.2 Hz, 2H) , 2.02 (s, 3H), 1.44 (d, J = 6.8 Hz, 6H). | m/z: 499.3 [M + H]⁺ |
| | 2-(1-isopropyl-4-methyl-1H- | | |
| | pyrazol-5-yl)-4-(4-(1-methyl- | | |
| | 3-(trifluoromethyl)-1H-1,2,4- | | |
| | triazol-5-yl)benzyl)-6,7- | | |
| | dihydropyrazolo[1,5- | | |
| | a]pyrimidin-5(4H)-one | | |
| 12 | 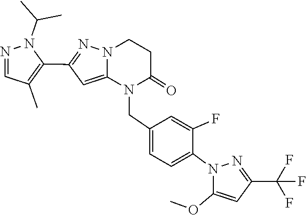 [SMILES: COc1cc(C(F)(F)F)nn1-c1ccc(CN2C(=O)CCn3nc(-c4c(C)cnn4C(C)C)cc32)cc1F] | ¹H NMR (400 MHz, Methanol-d₄) δ 7.49-7.35(m, 4H), 6.20 (s, 1H), 6.04 (s, 1H), 5.14 (s, 2H), 4.86 (s, 1H), 4.48 (t, J = 7.2 Hz, 2H) , 3.96 (s, 3H), 3.16 (t, J = 12 Hz, 2H) , 2.03 (s, 3H), 1.36 (d, J = 6.4 Hz, 6H). | m/z: 532.3 [M + H]⁺ |
| | 4-(3-fluoro-4-(5-methoxy-3- | | |
| | (trifluoromethyl)-1H-pyrazol- | | |
| | 1-yl)benzyl)-2-(1-isopropyl-4- | | |
| | methyl-1H-pyrazol-5-yl)-6,7- | | |
| | dihydropyrazolo[1, 5- | | |
| | a]pyrimidin-5(4H)-one | | |
| 13 | 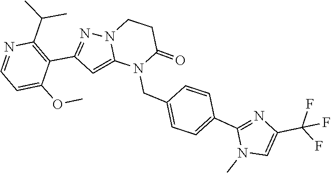 [SMILES: COc1ccnc(C(C)C)c1-c1cc2n(n1)CCC(=O)N2Cc1ccc(-c2nc(C(F)(F)F)cn2C)cc1] | ¹H NMR (400 MHz, Methanol-d₄) δ 8.62 (d, J = 6.8 Hz, 1H), 7.69 (s, 1H), 7.66 (d, J = 8.4 Hz, 2H), 7.55 (t, J = 6.8 Hz, 3H), 6.04 (s, 1H), 5.12 (s, 2H), 4.49 (t, J = 6.8 Hz, 2H) , 4.04 (s, 3H), 3.78 (s, 3H), 3.42-3.35 (m, 1H), 3.18 (t, J = 7.2 Hz, 2H) , 1.30 (d, J = 6.8 Hz, 6H). | m/z: 525.2 [M + H]⁺ |
| | 2-(2-isopropyl-4- | | |
| | methoxypyridin-3-yl)-4-(4-(1- | | |
| | methyl-4-(trifluoromethyl)- | | |
| | 1H-imidazol-2-yl)benzyl)-6,7- | | |
| | dihydropyrazolo[1,5- | | |
| | a]pyrimidin-5(4H)-one | | |
| 14 | 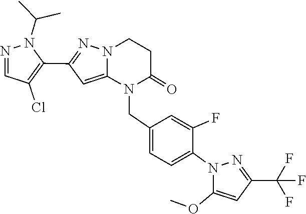 [SMILES: COc1cc(C(F)(F)F)nn1-c1ccc(CN2C(=O)CCn3nc(-c4c(Cl)cnn4C(C)C)cc32)cc1F] | ¹H NMR (400 MHz, Chloroform-d) δ 7.49 (s, 1H), 7.45 (t, J = 8.0 Hz, 1H), 7.24 (t, J = 8.0 Hz, 2H), 6.14 (s, 1H), 5.91 (s, 1H), 5.18 (m, 1H), 5.02 (s, 2H), 4.44 (t, J = 7.2 Hz, 2H), 3.93 (s, 3H), 3.09 (t, J = 7.2 Hz, 2H), 1.45 (d, J = 6.4 Hz, 6H). | m/z: 552.2 [M + H]⁺ |
| | 2-(4-chloro-1-isopropyl-1H- | | |
| | pyrazol-5-yl)-4-(3-fluoro-4- | | |
| | (5-methoxy-3- | | |
| | (trifluoromethyl)-1H-pyrazol- | | |
| | 1-yl)benzyl)-6,7- | | |
| | dihydropyrazolo[1,5- | | |
| | a]pyrimidin-5(4H)-one | | |
| 15 | 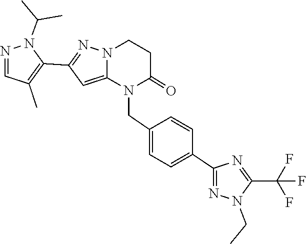 [SMILES: CCn1nc(-c2ccc(CN3C(=O)CCn4nc(-c5c(C)cnn5C(C)C)cc43)cc2)nc1C(F)(F)F] | ¹H NMR (400 MHz, Chloroform-d) δ 7.96 (d, J = 8.4 Hz, 2H), 7.29 (d, J = 8.4 Hz, 2H), 7.17 (s, 1H), 5.67 (s, 1H), 4.95 (s, 2H), 4.77 (m, 1H), 4.31 (m, 4H), 3.03 (t, J = 6.8 Hz, 2H), 1.92 (s, 3H), 1.47 (t, J = 7.2 Hz, 3H), 1.32 (d, J = 6.8 Hz, 6H). | m/z: 513.2 [M + H]⁺ |
| | 4-(4-(1-ethyl-5- | | |
| | (trifluoromethyl)-1H-1, 2, 4- | | |
| | triazol-3-yl)benzyl)-2-(1- | | |
| | isopropyl-4-methyl-1H- | | |
| | pyrazol-5-yl)-6,7- | | |
| | dihydropyrazolo[1,5- | | |
| | a]pyrimidin-5(4H)-one | | |
| 16 | 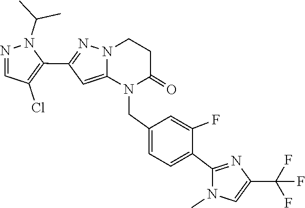 [SMILES: CC(C)n1ncc(Cl)c1-c1cc2n(n1)CCC(=O)N2Cc1ccc(-c2nc(C(F)(F)F)cn2C)c(F)c1] | ¹H NMR (400 MHz, Chloroform-d) δ 7.59 (t, J = 7.6 Hz, 1H), 7.48 (s, 1H), 7.34 (s, 1H), 7.24 (d, J = 1.6 Hz, 1H), 7.19-7.13 (m, 1H), 6.14 (s, 1H), 5.22-5.15(m, 1H), 5.04 (s, 2H), 4.45 (t, J = 7.1 Hz, 2H), 3.62 (d, J = 2.3 Hz, 3H), 3.11 (t, J = 7.0 Hz, 2H), 1.45 (d, J = 6.6 Hz, 6H). | m/z: 536.2 [M + H]⁺ |
| | 2-(4-chloro-1-isopropyl-1H- | | |
| | pyrazol-5-yl)-4-(3-fluoro-4- | | |
| | (1-methyl-4-(trifluoromethyl)- | | |
| | 1H-imidazol-2-yl)benzyl)-6,7- | | |
| | dihydropyrazolo[1,5- | | |
| | a]pyrimidin-5(4H)-one | | |
| 17 | 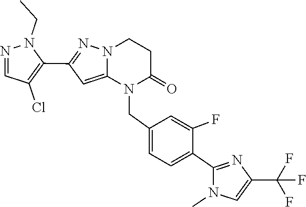 [SMILES: CCn1ncc(Cl)c1-c1cc2n(n1)CCC(=O)N2Cc1ccc(-c2nc(C(F)(F)F)cn2C)c(F)c1] | ¹H NMR (400 MHz, Chloroform-d) δ 7.60 (t, J = 7.6 Hz, 1H), 7.46 (s, 1H), 7.36-7.33 (m, 1H), 7.24 (s, 1H), 7.16 (d, J = 10.6 Hz, 1H), 6.19 (s, 1H), 5.04 (s, 2H), 4.50-4.41 (m, 4H), 3.63 (d, J = 2.3 Hz, 3H), 3.11 (t, J = 7.1 Hz, 2H), 1.40 (t, J = 7.2 Hz, 3H). | m/z: 522.2 [M + H]⁺ |
| | 2-(4-chloro-1-ethyl-1H- | | |
| | pyrazol-5-yl)-4-(3-fluoro-4- | | |
| | (1-methyl-4-(trifluoromethyl)- | | |
| | 1H-imidazol-2-yl)benzyl)-6,7- | | |
| | dihydropyrazolo[1,5- | | |
| | a]pyrimidin-5(4H)-one | | |
| 18 | 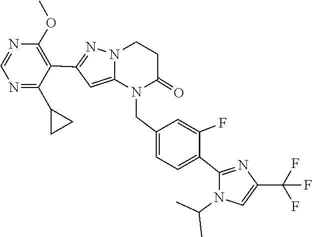 [SMILES: COc1ncnc(C2CC2)c1-c1cc2n(n1)CCC(=O)N2Cc1ccc(-c2nc(C(F)(F)F)cn2C(C)C)c(F)c1] | ¹H NMR (400 MHz, Chloroform-d) δ 8.57 (s, 1H), 7.53 (t, J = 7.6 Hz, 1H), 7.43 (s, 1H), 7.16 (d, J = 10.5 Hz, 1H), 5.86 (s, 1H), 5.03 (s, 2H), 4.47 (t, J = 7.1 Hz, 2H), 4.28-4.20 (m, 1H), 3.93 (s, 3H), 3.12 (t, J = 7.1 Hz, 2H), 2.21 (dt, J = 8.0, 3.5 Hz, 1H), 1.41 (d, J = 6.7 Hz, 6H), 1.18 (dq, J = 6.4, 3.7 Hz, 2H), 0.94 (dq, J = 6.9, 3.8 Hz, 2H). | m/z: 570.2 [M + H]⁺ |
| | 2-(4-cyclopropyl-6- | | |
| | methoxypyrimidin-5-yl)-4-(3- | | |
| | fluoro-4-(1-isopropyl-4- | | |
| | (trifluoromethyl)-1H- | | |
| | imidazol-2-yl)benzyl)-6,7- | | |
| | dihydropyrazolo[1,5- | | |
| | a]pyrimidin-5(4H)-one | | |
| 19 | 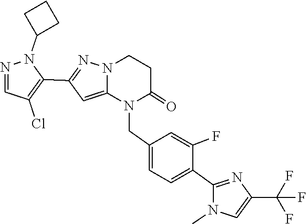 [SMILES: Cn1cc(C(F)(F)F)nc1-c1ccc(CN2C(=O)CCn3nc(-c4c(Cl)cnn4C4CCC4)cc32)cc1F] | ¹H NMR (400 MHz, Chloroform-d) δ 7.59 (t, J = 7.6 Hz, 1H), 7.51 (s, 1H), 7.34 (s, 1H), 7.24 (s, 1H), 7.15 (d, J = 10.5 Hz, 1H), 6.11 (s, 1H), 5.40-5.29 (m, 1H), 5.04 (s, 2H), 4.47 (t, J = 7.1 Hz, 2H), 3.62 (d, J = 2.1 Hz, 3H), 3.12 (t, J = 7.1 Hz, 2H), 2.73-2.63 (m, 2H), 2.35 (q, J = 8.1 Hz, 2H), 1.92-1.73 (m, 2H). | m/z: 548.2 [M + H]⁺ |
| | 2-(4-chloro-1-cyclobutyl-1H- | | |
| | pyrazol-5-yl)-4-(3-fluoro-4- | | |
| | (1-methyl-4-(trifluoromethyl)- | | |
| | 1H-imidazol-2-yl)benzyl)-6,7- | | |
| | dihydropyrazolo[1,5- | | |
| | a]pyrimidin-5(4H)-one | | |
| 20 | 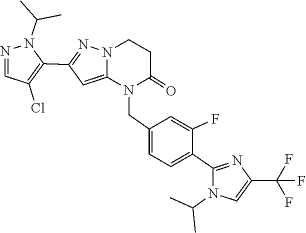 [SMILES: CC(C)n1cc(C(F)(F)F)nc1-c1ccc(CN2C(=O)CCn3nc(-c4c(Cl)cnn4C(C)C)cc32)cc1F] | ¹H NMR (400 MHz, Chloroform-d) δ 7.52 (t, J = 7.5 Hz, 1H), 7.49 (s, 1H), 7.43 (s, 1H), 7.25 (d, J = 10.3 Hz, 1H), 7.15 (d, J = 10.2 Hz, 1H), 6.14 (s, 1H), 5.22-5.15 (m, 1H), 5.04 (s, 2H), 4.45 (t, J = 6.9 Hz, 2H), 4.23 (m, 1H), 3.11 (t, J = 6.9 Hz, 2H), 1.46 (d, J = 6.6 Hz, 6H), 1.40 (d, J = 6.6 Hz, 6H). | m/z: 564.2 [M + H]⁺ |
| | 2-(4-chloro-1-isopropyl-1H- | | |
| | pyrazol-5-yl)-4-(3-fluoro-4- | | |
| | (1-isopropyl-4- | | |
| | (trifluoromethyl)-1H- | | |
| | imidazol-2-yl)benzyl)-6,7- | | |
| | dihydropyrazolo[1,5- | | |
| | a]pyrimidin-5(4H)-one | | |
| 21 | 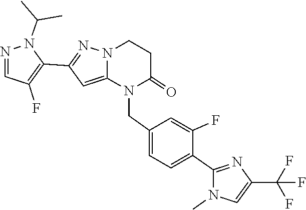 [SMILES: CC(C)n1ncc(F)c1-c1cc2n(n1)CCC(=O)N2Cc1ccc(-c2nc(C(F)(F)F)cn2C)c(F)c1] | ¹H NMR (400 MHz, Chloroform-d) δ 7.60 (t, J = 7.4 Hz, 1H), 7.38 (d, J = 4.5 Hz, 1H), 7.35 (s, 1H), 7.24 (d, J = 8.4 Hz, 1H), 7.14 (d, J = 10.6 Hz, 1H), 5.98 (s, 1H), 5.40-5.23 (m, 1H), 5.03 (s, 2H), 4.44 (t, J = 7.1 Hz, 2H), 3.63 (s, 3H), 3.11 (t, J = 6.9 Hz, 2H), 1.46 (d, J = 6.5 Hz, 6H). | m/z: 520.2 [M + H]⁺ |
| | 2-(4-fluoro-1-isopropyl-1H- | | |
| | pyrazol-5-yl)-4-(3-fluoro-4- | | |
| | (1-methyl-4-(trifluoromethyl)- | | |
| | 1H-imidazol-2-yl)benzyl)-6,7- | | |
| | dihydropyrazolo[1,5- | | |
| | a]pyrimidin-5(4H)-one | | |
| 22 | 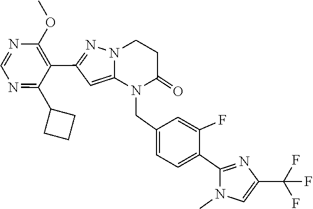 [SMILES: COc1ncnc(C2CCC2)c1-c1cc2n(n1)CCC(=O)N2Cc1ccc(-c2nc(C(F)(F)F)cn2C)c(F)c1] | ¹H NMR (400 MHz, Chloroform-d) δ 8.78 (s, 1H), 7.60 (t, J = 7.5 Hz, 1H), 7.35 (s, 1H), 7.24 (s, 1H), 7.15 (d, J = 10.6 Hz, 1H), 5.73 (s, 1H), 5.03 (s, 2H), 4.46 (t, J = 7.1 Hz, 2H), 3.93 (s, 3H), 3.80- 3.67 (m, 1H), 3.63 (d, J = 2.0 Hz, 3H), 3.13 (t, J = 7.0 Hz, 2H), 2.48-2.30 (m, 2H), 2.04 (d, J = 8.3 Hz, 2H), 1.96-1.81 (m, 2H). | m/z: 556.2 [M + H]⁺ |
| | 2-(4-chloro-1-isopropyl-1H- | | |
| | pyrazol-5-yl)-4-(4-(1-ethyl-3- | | |
| | (trifluoromethyl)-1H-1,2,4- | | |
| | triazol-5-yl)benzyl)-6,7- | | |
| | dihydropyrazolo[1,5- | | |
| | a]pyrimidin-5(4H)-one | | |
| 23 | 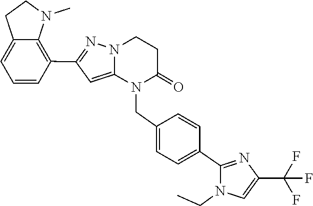 [SMILES: CCn1cc(C(F)(F)F)nc1-c1ccc(CN2C(=O)CCn3nc(-c4cccc5c4N(C)CC5)cc32)cc1] | ¹H NMR (400 MHz, Methanol-d₄) δ 7.78 (d, J = 1.3 Hz, 1H), 7.60-7.52 (m, 4H), 7.19-7.10 (m, 2H), 6.85 (t, J = 7.5 Hz, 1H), 6.05 (s, 1H), 5.12 (s, 2H), 4.43 (t, J = 7.1 Hz, 2H), 4.09 (q, J = 7.3 Hz, 2H), 3.40 (t, J = 8.1 Hz, 2H), 3.14 (t, J = 7.1 Hz, 2H), 3.02 (t, J = 8.1 Hz, 2H), 2.57 (s, 3H), 1.35 (t, J = 7.3 Hz, 3H). | m/z: 521.2 [M + H]⁺ |
| | 4-(4-(1-ethyl-4- | | |
| | (trifluoromethyl)-1H- | | |
| | imidazol-2-yl)benzyl)-2-(1- | | |
| | methylindolin-7-yl)-6,7- | | |
| | dihydropyrazolo[1,5- | | |
| | a]pyrimidin-5(4H)-one | | |

### Example 24. 2-(4-cyclopropyl-6-methoxypyrimidin-5-yl)-4-(4-(1-methyl-4-(trifluoromethyl)-1H-imidazol-2-yl)benzyl)-4,5,6,7-tetrahydropyrazolo[1,5-α]pyrimidine

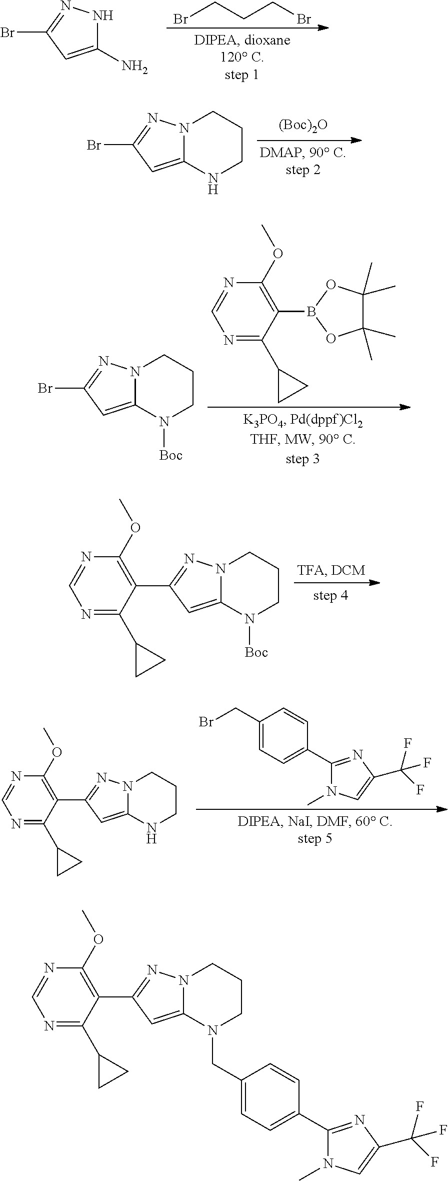

### Step 1. 2-bromo-4,5,6,7-tetrahydropyrazolo[1,5-α]pyrimidine

To a solution of 3-bromo-1H-pyrazol-5-amine (1 g, 6.17 mmol) in dry dioxane (25 mL) was added 1,3-dibromopropane (1.5 g, 7.41 mmol) and DIPEA (2.4 g, 18.52 mmol). The resulting solution was heated at 120° C. for 12 h in a sealed tube. The solvent was removed under vacuum and the residue was diluted with brine (100 mL) and extracted with EtOAc (100 mL×3). The organic layers were combined, dried over anhydrous Na₂SO₄, filtered, concentrated under vacuum and purified on a Biotage Isolera One (C₁₈ column, eluting with 10% to 95% MeCN/H₂O) to afford 2-bromo-4,5,6,7-tetrahydropyrazolo[1,5-α]pyrimidine (285 mg). ¹H NMR (400 MHz, DMSO-d₆) δ 6.25 (s, 1H), 5.24 (s, 1H), 3.90 (t, J=6.1 Hz, 2H), 3.16-3.08 (m, 2H), 1.95 (m, 2H). MS (ESI) m/z 202.3, 204.3 [M+H]⁺

### Step 2. tert-butyl 2-bromo-6,7-dihydropyrazolo[1,5-α]pyrimidine-4(5H)-carboxylate

A mixture of 2-bromo-4,5,6,7-tetrahydropyrazolo[1,5-α]pyrimidine (285 mg, 1.42 mmol), di-tert-butyl dicarbonate (464 mg, 2.13 mmol) and DMAP (260 mg, 2.13 mmol) was heated at 90° C. for 1 h. The reaction mixture was cooled to room temperature and purified by silica gel chromatography (eluting with 1/4 EtOAc/PE) to afford tert-butyl 2-bromo-6,7-dihydropyrazolo[1,5-α]pyrimidine-4(5H)-carboxylate (352 mg). MS (ESI) m/z 302.1, 304.1 [M+H]⁺.

### Step 3. tert-butyl 2-(4-cyclopropyl-6-methoxypyrimidin-5-yl)-6,7-dihydropyrazolo[1,5-α]pyrimidine-4(5H)-carboxylate

To a solution of tert-butyl 2-bromo-6,7-dihydropyrazolo[1,5-α]pyrimidine-4(5H)-carboxylate (350 mg, 1.16 mmol) in THF (10 mL) was added 4-cyclopropyl-6-methoxy-5-(4,4,5,5-tetramethyl-1,3,2-dioxaborolan-2-yl) pyrimidine (385 mg, 1.40 mmol), K₃PO₄ (492 mg, 2.32 mmol) and Pd(dppf)Cl₂ (88 mg, 0.12 mmol). The resulting solution was heated at 90° C. for 1 h in microwave reactor. The reaction mixture was concentrated and purified by silica gel chromatography (eluting with 1/100 MeOH/DCM) to afford tert-butyl 2-(4-cyclopropyl-6-methoxypyrimidin-5-yl)-6,7-dihydropyrazolo[1,5-α]pyrimidine-4(5H)-carboxylate (508 mg). MS (ESI) m/z 372.3, 373.3 [M+H]⁺.

### Step 4. 2-(4-cyclopropyl-6-methoxypyrimidin-5-yl)-4,5,6,7-tetrahydropyrazolo[1,5-α]pyrimidine

To a solution of tert-butyl 2-(4-cyclopropyl-6-methoxypyrimidin-5-yl)-6,7-dihydropyrazolo[1,5-α]pyrimidine-4(5H)-carboxylate (500 mg, 1.34 mmol) in DCM (4 mL) at 0° C. was added TFA (4 mL) slowly. The resulting solution was stirred at room temperature for 3 h. The resulting solution was concentrated under vacuum, the residue obtained was dissolved in EtOAc (50 mL) and washed with 1M NaOH aqueous solution. The organic phase was concentrated under vacuum and purified on a Biotage Isolera One (C₁₈ column, eluting with 10% to 95% MeCN/H₂O) to afford 2-(4-cyclopropyl-6-methoxypyrimidin-5-yl)-4,5,6,7-tetrahydropyrazolo[1,5-α]pyrimidine (220 mg). MS (ESI) m/z 272.9 [M+H]⁺.

### Step 5. 2-(4-cyclopropyl-6-methoxypyrimidin-5-yl)-4-(4-(1-methyl-4-(trifluoromethyl)-1H-imidazol-2-yl)benzyl)-4,5,6,7-tetrahydropyrazolo[1,5-α]pyrimidine

To a solution of 2-(4-cyclopropyl-6-methoxypyrimidin-5-yl)-4,5,6,7-tetrahydropyrazolo[1,5-α]pyrimidine (120 mg, 0.44 mmol) and 2-(4-(bromomethyl)phenyl)-1-methyl-4-(trifluoromethyl)-1H-imidazole (293 mg, 0.88 mmol) in DMF (3 mL) was added NaI (132 mg, 0.88 mmol) and DIPEA (114 mg, 0.88 mmol). The resulting solution was heated at 60° C. for 12 h. The reaction mixture was dissolved in EtOAc (30 mL), washed with brine (3×30 mL). The organic phase was dried with Na₂SO₄, concentrated under vacuum and purified by prep-HPLC (condition 4) to afford Example 24 2-(4-cyclopropyl-6-methoxypyrimidin-5-yl)-4-(4-(1-methyl (trifluoromethyl)-1H-imidazol-2-yl) benzyl)-4,5,6,7-tetrahydropyrazolo[1,5-α]pyrimidine (25.5 mg).

Table 2. The compounds listed in Table 2 were synthesized according to Example 24 using the appropriate commercially available reagents and/or intermediates described above.

TABLE 2

| Example | | | |
|---|---|---|---|
| number | Structure | ¹H NMR (δ ppm) | MS (ESI) |
| 24 | 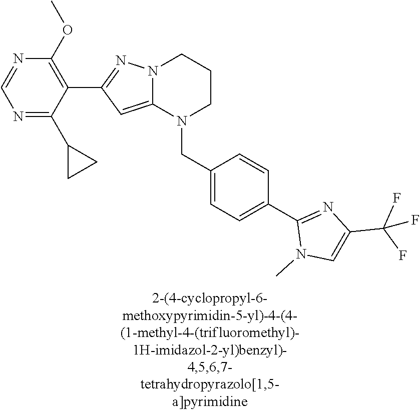 [SMILES: COc1ncnc(C2CC2)c1-c1cc2n(n1)CCCN2Cc1ccc(-c2nc(C(F)(F)F)cn2C)cc1] | ¹H NMR (400 MHz, Methanol-d₄) δ 8.61 (s, 1H), 7.74 (s, 1H), 7.69 (d, J = 7.2 Hz, 2H), 7.56 (d, J = 7.2 Hz, 2H), 4.94 (s, 1H), 4.66 (s, 2H), 4.24 (t, J = 7.2 Hz, 2H), 3.98 (s, 3H), 3.80 (s, 3H), 3.44-3.42 (m, 2H), 2.32-2.35 (m, 2H), 2.09-2.11 (m, 1H), 1.18-1.20 (m, 2H), 1.03-1.07 (m, 2H). | m/z: 510.4 [M + H]⁺ |
| 25 | 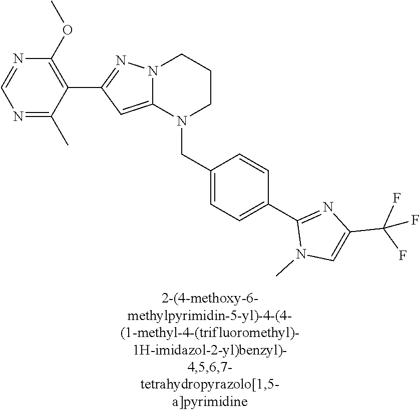 [SMILES: COc1ncnc(C)c1-c1cc2n(n1)CCCN2Cc1ccc(-c2nc(C(F)(F)F)cn2C)cc1] | ¹H NMR (400 MHz, Methanol-d₄) δ 8.92 (s, 1H), 7.74 (s, 1H), 7.69 (d, J = 7.2 Hz, 2H), 7.56 (d, J = 7.2 Hz, 2H), 4.92 (s, 1H), 4.57 (s, 2H), 4.21-4.18 (t, J = 6.8 Hz, 2H), 4.12 (s, 3H), 3.80 (s, 3H), 3.36 (t, J = 6.0 Hz, 2H), 2.59 (s, 3H), 2.32-2.29 (m, 2H). | m/z: 484.2 [M + H]⁺ |
| 26 | 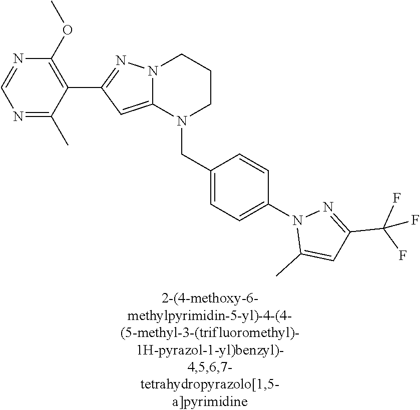 [SMILES: COc1ncnc(C)c1-c1cc2n(n1)CCCN2Cc1ccc(-n2nc(C(F)(F)F)cc2C)cc1] | ¹H NMR (400 MHz, Methanol-d₄) δ 8.88 (s, 1H), 7.59-7.50 (m, 5H), 6.59 (s, 1H), 4.57 (s, 2H), 4.18 (t, J = 6.4 Hz, 2H), 4.10 (s, 3H), 3.36 (t, J = 5.6 Hz, 2H), 2.57 (s, 3H), 2.36 (s, 3H), 2.33-2.19 (m, 2H). | m/z: 484.3 [M + H]⁺ |
| 27 | 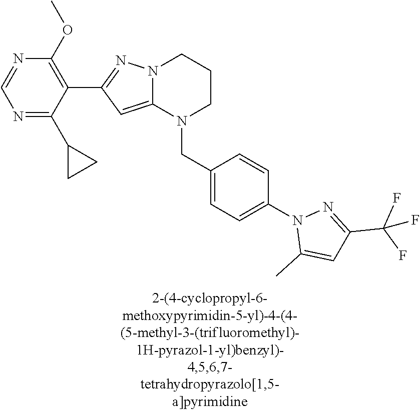 [SMILES: COc1ncnc(C2CC2)c1-c1cc2n(n1)CCCN2Cc1ccc(-n2nc(C(F)(F)F)cc2C)cc1] | ¹H NMR (400 MHz, Methanol-d₄) δ 8.60 (s, 1H), 7.61-7.51 (m, 5H), 6.59 (s, 1H), 4.65 (s, 2H), 4.23 (t, J = 6.0 H, 2H), 3.97 (s, 3H), 3.43 (t, J = 5.6 Hz, 2H), 2.36 (s, 3H), 2.34-2.30 (m, 2H), 2.16- 1.08 (m, 1H), 1.20-1.15 (m, 2H), 1.06-1.00 (m, 2H). | m/z: 510.3 [M + H]⁺ |
| 28 | 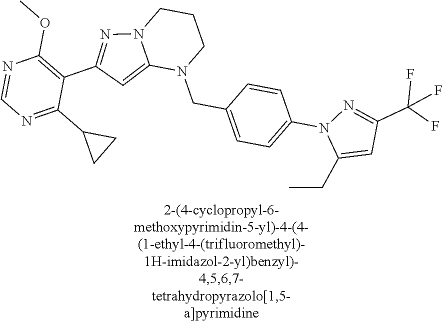 [SMILES: CCc1cc(C(F)(F)F)nn1-c1ccc(CN2CCCn3nc(-c4c(OC)ncnc4C4CC4)cc32)cc1] | ¹H NMR (400 MHz, Chloroform-d) δ 8.55 (s, 1H), 7.58 (d, J = 8.0 Hz, 2H), 7.45 (d, J = 8.0 Hz, 2H), 7.37 (s, 1H), 5.56 (s, 1H), 4.38 (s, 2H), 4.21 (t, J = 10.0 Hz, 2H), 4.08 (q, J = 6.8 Hz, 2H), 3.94 (s, 3H), 3.18-3.15 (m, 2H), 2.36-2.34 (m, 1H), 2.33-2.27 (m, 2H), 1.46-1.42 (m, 3H), 1.19-1.17 (m, 2H), 0.97-0.92 (m, 2H). | m/z: 524.2 [M + H]⁺ |
| 29 | 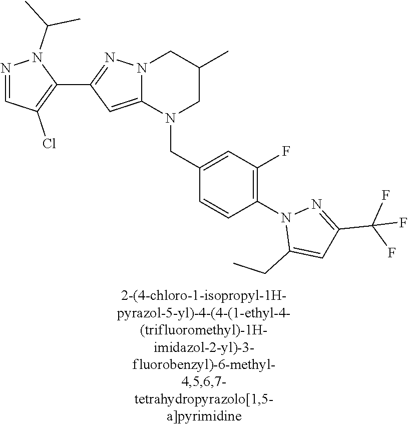 [SMILES: CCc1cc(C(F)(F)F)nn1-c1ccc(CN2CC(C)Cn3nc(-c4c(Cl)cnn4C(C)C)cc32)cc1F] | ¹H NMR (400 MHz, Chloroform-d) δ 7.56 (t, J = 7.6 Hz, 1H), 7.47 (s, 1H), 7.41 (d, J = 1.0 Hz, 1H), 7.26- 7.22 (m, 1H), 7.18 (d, J = 10.6 Hz, 1H), 5.77 (s, 1H), 5.26 (m, 1H), 4.46-4.32 (m, 2H), 4.26 (dd, J = 12.4, 4.4 Hz, 1H), 3.93 (q, J = 7.3 Hz, 2H), 3.71 (dd, J = 12.3, 9.8 Hz, 1H), 3.17-3.11 (m, 1H), 2.89- 2.82 (m, 1H), 2.53-2.43 (m, 1H), 1.45 (dd, J = 6.6, 3.8 Hz, 6H), 1.40 (t, J = 7.3 Hz, 3H), 1.14 (d, J = 6.7 Hz, 3H). | m/z: 550.3 [M + H]⁺ |
| 30 | 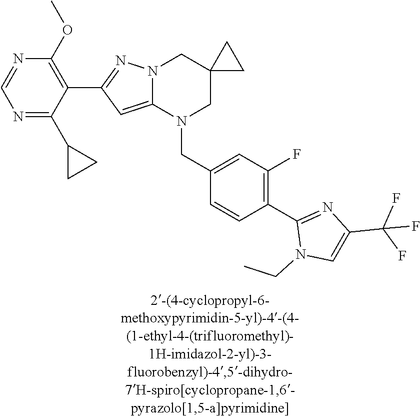 [SMILES: CCn1cc(C(F)(F)F)nc1-c1ccc(CN2CC3(CC3)Cn3nc(-c4c(OC)ncnc4C4CC4)cc32)cc1F] | ¹H NMR (400 MHz, Chloroform-d) δ 8.55 (s, 1H), 7.56 (t, J = 7.6 Hz, 1H), 7.42- 7.41 (m, 1H), 7.27 (d, J = 6.2 Hz, 1H), 7.22 (d, J = 10.6 Hz, 1H), 5.59 (s, 1H), 4.36 (s, 2H), 4.00 (s, 2H), 3.94 (d, J = 7.5 Hz, 5H), 2.88 (s, 2H), 2.37 (tt, J = 8.3, 4.7 Hz, 1H), 1.40 (t, J = 7.3 Hz, 3H), 1.16 (dt, J = 6.3, 3.2 Hz, 2H), 0.94 (dq, J = 6.8, 3.7 Hz, 2H), 0.80-0.75 (m, 2H), 0.69-0.64 (m, 2H). | m/z: 568.2 [M + H]⁺ |

### Example 31. 2-(1-isopropyl-4-methyl-1H-pyrazol-5-yl)-6-methyl-4-(4-(1-methyl-4-(trifluoromethyl)-1H-imidazol-2-yl)benzyl)-6,7-dihydropyrazolo[1,5-α]pyrimidin-5(4H)-one

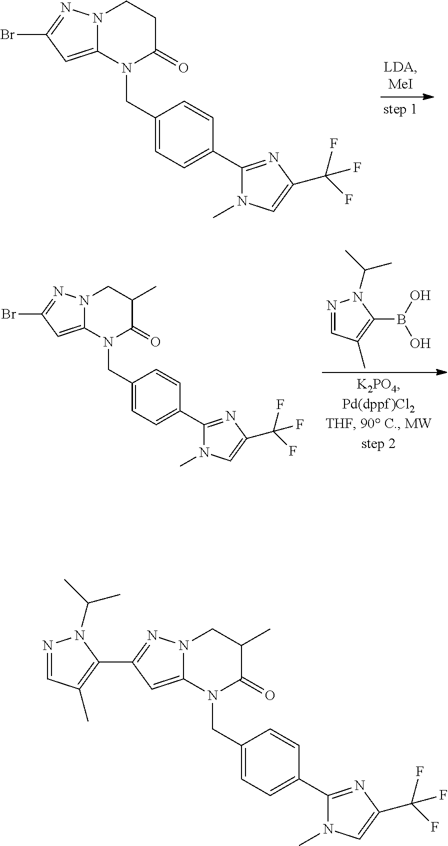 [SMILES: Cn1cc(C(F)(F)F)nc1-c1ccc(CN2C(=O)CCn3nc(Br)cc32)cc1 ; CC1Cn2nc(Br)cc2N(Cc2ccc(-c3nc(C(F)(F)F)cn3C)cc2)C1=O ; Cc1cnn(C(C)C)c1B(O)O ; Cc1cnn(C(C)C)c1-c1cc2n(n1)CC(C)C(=O)N2Cc1ccc(-c2nc(C(F)(F)F)cn2C)cc1]

### Step 1. 2-bromo-6-methyl-4-(4-(1-methyl-4-(trifltmromethyl)-1H-imidazol-2-yl)benzyl)-6,7-dihydropyrazolo[1,5-α]pyrimidin-5(4H)-one

A solution of 2-bromo-4-(4-(1-methyl-4-(trifluoromethyl)-1H-imidazol-2-yl)benzyl)-6,7-dihydropyrazolo[1,5-α]pyrimidin-5(4H)-one (300 mg, 0.66 mmol, prepared in step 2 of Example 1) in dry THF (8 mL) was cooled to −78° C. LDA (0.33 mL, 2 M) was added dropwise and the solution stirred at −78° C. for 30 min then a solution of MeI (84.3 mg, 0.59 mmol) in dry THF (0.5 mL) was added. The resulting solution was stirred at −78° C. for another 30 min then warmed to room temperature. The reaction was quenched with water, diluted with EtOAc, washed with water and brine. The organic phase was dried with Na₂SO₄, concentrated, and purified by silica gel chromatography (eluting with 2:1 PE/EtOAc) to afford 2-bromo-6-methyl-4-(4-(1-methyl-4-(trifluoromethyl)-1H-imidazol-2-yl)benzyl)-6,7-dihydropyrazolo[1,5-α]pyrimidin-5(4H)-one (70 mg). MS (ESI) m/z 468.1, 470.1 [M+H]⁺.

### Step 2. 2-(1-isopropyl-4-methyl-1H-pyrazol-5-yl)-6-methyl-4-(4-(1-methyl-4-(trifluoromethyl)-1H-imidazol-2-yl) benzyl)-6,7-dihydropyrazolo[1,5-α]pyrimidin-5(4H)-one

Example 31 2-(1-isopropyl-4-methyl-1H-pyrazol-5-yl)-6-methyl-4-(4-(1-methyl-4-(trifluoromethyl)-1H-imidazol-2-yl)benzyl)-6,7-dihydropyrazolo[1,5-α]pyrimidin-5(4H)-one was synthesized from (1-isopropyl-4-methyl-1H-pyrazol-5-yl)boronic acid (intermediate 3) and 2-bromo-6-methyl-4-(4-(1-methyl-4-(trifluoromethyl)-1H-imidazol-2-yl)benzyl)-6,7-dihydropyrazolo[1,5-α]pyrimidin-5(4H)-one following step 3 of Example 24.

Table 3. The compounds listed in Table 3 were synthesized according to Example 31 using the appropriate commercially available reagents and/or intermediates described above.

TABLE 3

| Example | | | |
|---|---|---|---|
| number | Structure | ¹H NMR (δ ppm) | MS (ESI) |
| 31 | 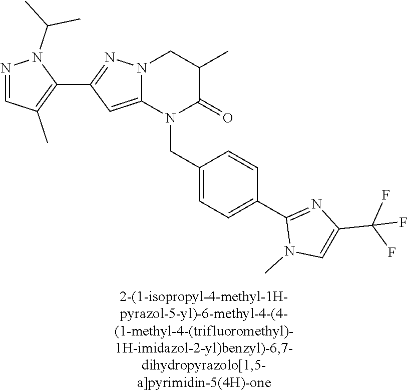 [SMILES: Cc1cnn(C(C)C)c1-c1cc2n(n1)CC(C)C(=O)N2Cc1ccc(-c2nc(C(F)(F)F)cn2C)cc1] | ¹H NMR (400 MHz, Chloroform-d) δ 7.58 (d, J = 8.0 Hz, 2H), 7.41 (d, J = 8.0 Hz, 2H), 7.36 (s, 1H), 7.29 (s, 1H), 5.76 (s, 1H), 5.07-4.98 (m, 2H), 4.94-4.87 (m, 1H), 4.52 (dd, J = 13.2, 6.4 Hz, 1H), 4.07 (d, J = 12.8, 10.0 Hz, 1H), 3.74 (s, 3H), 3.17- 3.11 (m, 1H), 2.02 (s, 3H), 1.44-1.42 (m, 9H). | m/z: 512.2 [M + H]⁺ |
| 32 | 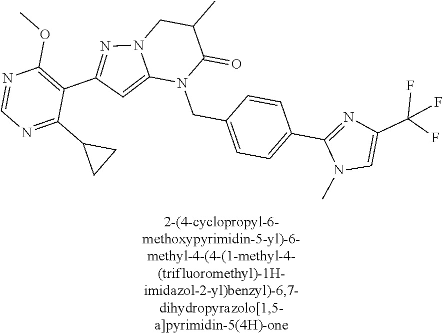 [SMILES: COc1ncnc(C2CC2)c1-c1cc2n(n1)CC(C)C(=O)N2Cc1ccc(-c2nc(C(F)(F)F)cn2C)cc1] | ¹H NMR (400 MHz, Methanol-d₄) δ 8.48 (s, 1H), 7.68 (s, 1H), 7.64 (d, J = 8.0 Hz, 2H), 7.52 (d, J = 8.0 Hz, 2H), 6.01 (s, 1H), 5.10 (s, 2H), 4.51-4.54 (m, 1H), 4.12 (t, J = 6.8 Hz, 1H), 3.87 (s, 3H), 3.76 (s, 3H), 3.25-3.20 (m, 1H), 2.17-2.13 (m, 1H), 1.39 (d, J = 6.8 Hz, 3H), 1.09- 1.06 (m, 2H), 0.91-0.87 (m, 2H). | m/z: 538.3 [M + H]⁺ |

### Example 33. 3-chloro-2-(4-cyclopropyl-6-methoxypyrimidin-5-yl)-4-(4-(1-methyl-4-(trifluoromethyl)-1H-imidazol-2-yl)benzyl)-6,7-dihydropyrazolo[1,5-α]pyrimidin-5(4H)-one

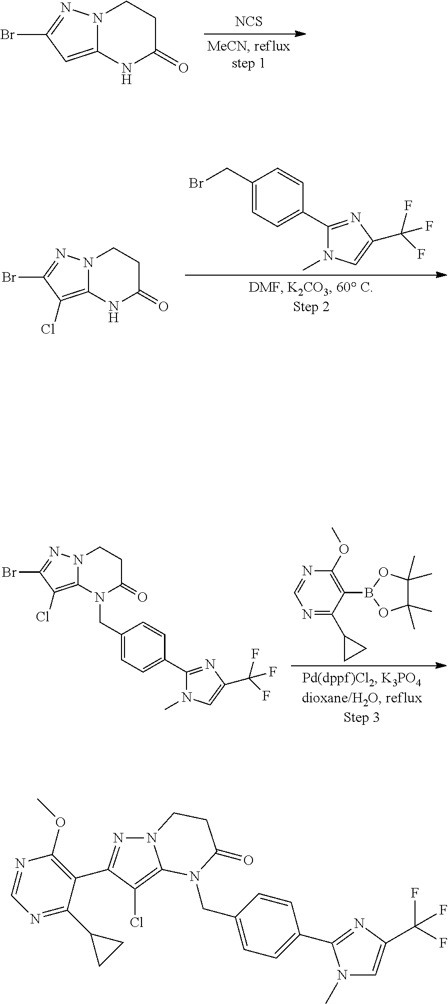

### Step 1. 2-bromo-3-chloro-6,7-dihydropyrazolo[1,5-α]pyrimidin-5(4H)-one

To a solution of 2-bromo-6,7-dihydropyrazolo[1,5-α]pyrimidin-5(4H)-one (200 mg, 0.93 mmol) in MeCN (10 mL) was added NCS (148 mg, 1.11 mmol). The resulting solution was heated at 80° C. for 2 h. The reaction mixture was concentrated, diluted with EtOAc and washed with brine. The organic layers were combined, dried over anhydrous Na₂SO₄, concentrated and purified on a Biotage Isolera One (C₁₈ column, eluting with 10% to 95% MeCN/H₂O) to afford 2-bromo-3-chloro-6,7-dihydropyrazolo[1,5-α]pyrimidin-5(4H)-one (200 mg). MS (ESI) m/z 250.0, 252.0 [M+H]⁺.

### Step 2. 2-bromo-3-chloro-4-(4-(1-methyl-4-(trifluoromethyl)-1H-imidazol-2-yl)benzyl)-6,7-dihydropyrazolo[1,5-α]pyrimidin-5(4H)-one

2-bromo-3-chloro-4-(4-(1-methyl-4-(trifluoromethyl)-1H-imidazol-2-yl)benzyl)-6,7-dihydropyrazolo[1,5-α]pyrimidin-5(4H)-one was synthesized from 2-bromo-3-chloro-6,7-dihydropyrazolo[1,5-α]pyrimidin-5(4H)-one and 2-(4-(bromomethyl)phenyl)-1-methyl (trifluoromethyl)-1H-imidazole (Intermediate BB-1) following step 2 of Example 1. MS (ESI) m/z 488.0, 490.0 [M+H]⁺.

### Step 3. 3-chloro-2-(4-cyclopropyl-6-methoxypyrimidin-5-yl)-4-(4-(1-methyl-4-(trifluoromethyl)-1H-imidazol-2-yl) benzyl)-6,7-dihydropyrazolo[1,5-α]pyrimidin-5(4H)-one

Example 33 3-chloro-2-(4-cyclopropyl-6-methoxypyrimidin-5-yl)-4-(4-(1-methyl-4-(trifluoromethyl)-1H-imidazol-2-yl)benzyl)-6,7-dihydropyrazolo[1,5-α]pyrimidin-5(4H)-one was synthesized from 2-bromo-3-chloro-4-(4-(1-methyl-4-(trifluoromethyl)-1H-imidazol-2-yl)benzyl)-6,7-dihydropyrazolo[1,5-α]pyrimidin-5(4H)-one according to step 3 of Example 1.

Table 4. The compounds listed in Table 4 were synthesized according to Example 33 using the appropriate commercially available reagents and/or intermediates described above. Enantiomers were not separated.

TABLE 4

| Example | | | |
|---|---|---|---|
| number | Structure | ¹H NMR (δ ppm) | MS (ESI) |
| 33 | 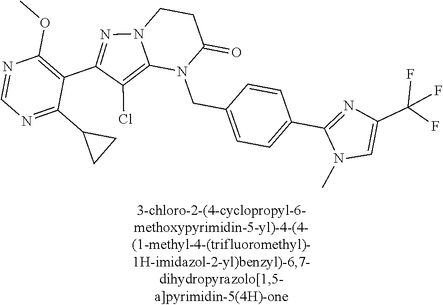 [SMILES: COc1ncnc(C2CC2)c1-c1nn2c(c1Cl)N(Cc1ccc(-c3nc(C(F)(F)F)cn3C)cc1)C(=O)CC2] | ¹H NMR (400 MHz, Methanol-d₄) δ 8.59 (s, 1H), 7.76 (s, 1H), 7.67 (d, J = 8.0 Hz, 2H), 7.52 (d, J = 8.0 Hz, 2H), 5.42 (s, 2H), 4.47 (t, J = 6.8 Hz, 2H), 3.91 (s, 3H), 3.79 (s, 3H), 3.14 (t, J = 6.8 Hz, 2H), 1.90-1.87 (m, 1H), 1.12- 1.10 (m, 2H), 0.97-0.94 (m, 2H). | m/z: 558.3 [M + H]⁺ |
| 34 | 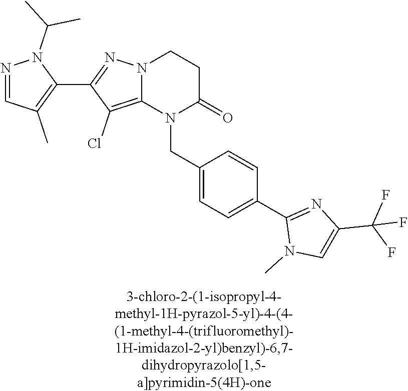 [SMILES: Cc1cnn(C(C)C)c1-c1nn2c(c1Cl)N(Cc1ccc(-c3nc(C(F)(F)F)cn3C)cc1)C(=O)CC2] | ¹H NMR (400 MHz, Methanol-d₄) δ 7.76 (s, 1H), 7.67 (d, J = 8.4 Hz, 2H), 7.52 (d, J = 8.0 Hz, 2H), 7.42 (s, 1H), 5.43 (s, 2H), 4.52 (t, J = 6.8 Hz, 2H), 4.40-4.34 (m, 1H), 3.78 (s, 3H), 3.17 (t, J = 7.2 Hz, 2H), 1.93 (s, 3H), 1.37 (d, J = 6.4 Hz, 6H). | m/z: 532.2 [M + H]⁺ |

### Example 35. 2-(4-cyclopropyl-6-methoxypyrimidin-5-yl)-3-methyl-4-(4-(1-methyl (trifluoromethyl)-1H-imidazol-2-yl)benzyl)-6,7-dihydropyrazol 0[1,5-α]pyrimidin-5(4H)-one

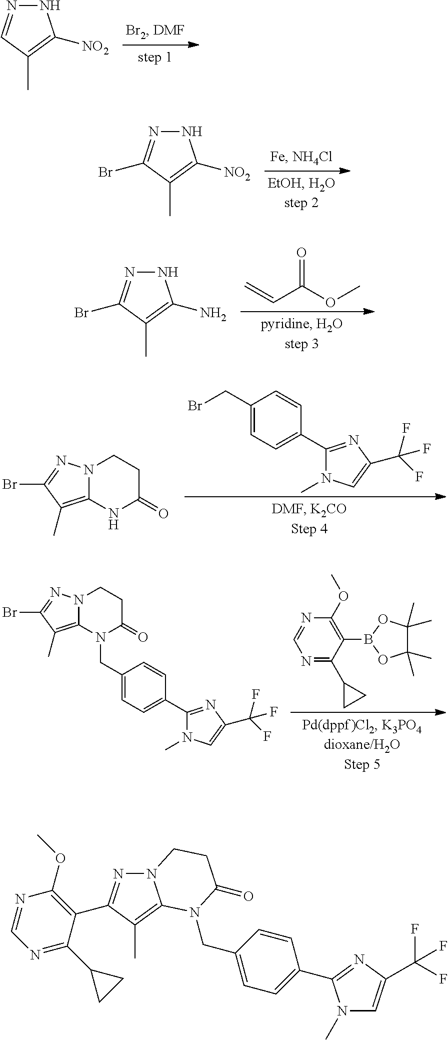

### Step 1. 3-bromo-4-methyl-5-nitro-1H-pyrazole

To a solution of 4-methyl-5-nitro-1H-pyrazole (350 mg, 2.75 mmol) in DMF (5 mL) was added Br₂ (878.9 mg, 5.5 mmol) and the resulting solution heated at 40° C. overnight. The reaction mixture was diluted with EtOAc and washed with brine. The organic layer was dried over anhydrous Na₂SO₄, concentrated and purified on a Biotage Isolera One (C₁₈ column, eluting with 10% to 95% MeCN/H₂O) to afford 3-bromo-4-methyl-5-nitro-1H-pyrazole (453 mg). MS (ESI) m/z 204.0, 206.0 [M-H]⁺.

### Step 2. 3-bromo-4-methyl-1H-pyrazol-5-amine

To a solution of 3-bromo-4-methyl-5-nitro-1H-pyrazole (450 mg, 2.19 mmol) in EtOH (10 mL) and H₂O (2 mL) was added Fe powder (614 mg, 10.97 mmol) and NH₄Cl (234 mg, 4.38 mmol). The resulting solution was heated at 90° C. for 30 min. The reaction mixture was filtered through celite and concentrated under vacuum. The crude was directly purified on a Biotage Isolera One (C₁₈ column, eluting with 10% to 95% MeCN/H₂O) to afford 3-bromo-4-methyl-1H-pyrazol-5-amine (251 mg). MS (ESI) m/z 176.0, 178.0 [M+H]⁺.

### Step 3. 2-bromo-3-methyl-6,7-dihydropyrazolo[1,5-α]pyrimidin-5(4H)-one

2-bromo-3-methyl-6,7-dihydropyrazolo[1,5-α]pyrimidin-5(4H)-one was synthesized from 3-bromo-4-methyl-1H-pyrazol-5-amine according to step 1 of Example 1. MS (ESI) m/z 230.0, 232.0 [M+H]⁺.

### Step 4. 2-bromo-3-methyl-4-(4-(1-methyl-4-(trifluoromethyl)-1H-imidazol-2-yl)benzyl)-6,7-dihydropyrazolo[1,5-α]pyrimidin-5(4H)-one

2-bromo-3-methyl-4-(4-(1-methyl-4-(trifluoromethyl)-1H-imidazol-2-yl)benzyl)-6,7-dihydropyrazolo[1,5-α]pyrimidin-5(4H)-one was prepared from 2-bromo-3-methyl-6,7-dihydropyrazolo[1,5-α]pyrimidin-5(4H)-one following step 2 of Example 1. MS (ESI) m/z 468.0, 470.0 [M+H]⁺.

### Step 5. 2-(4-cyclopropyl-6-methoxypyrimidin-5-yl)-3-methyl-4-(4-(1-methyl-4-(trifluoromethyl)-1H-imidazol-2-yl) benzyl)-6,7-dihydropyrazolo[1,5-α]pyrimidin-5(4H)-one

Example 35 2-(4-cyclopropyl-6-methoxypyrimidin-5-yl)-3-methyl-4-(4-(1-methyl-4-(trifluoromethyl)-1H-imidazol-2-yl)benzyl)-6,7-dihydropyrazolo[1,5-α]pyrimidin-5(4H)-one was prepared from 2-bromo-3-methyl-4-(4-(1-methyl-4-(trifluoromethyl)-1H-imidazol-2-yl)benzyl)-6,7-dihydropyrazolo[1,5-α]pyrimidin-5(4H)-one according to step 3 of Example 1. ¹H NMR (400 MHz, Methanol-d₄) δ 8.55 (s, 1H), 7.70 (s, 1H), 7.68 (d, J=8.0 Hz, 2H), 7.46 (d, J=8.0 Hz, 2H), 5.30 (s, 2H), 4.45 (t, J=6.8 Hz, 2H), 3.89 (s, 3H), 3.78 (s, 3H), 3.13 (t, J=6.4 Hz, 2H), 1.81-1.86 (m, 1H), 1.74 (s, 3H), 1.32-1.28 (m, 2H), 1.13-1.05 (m, 2H). MS (ESI) m/z 538.3 [M+H]⁺.

### Example 36. 2-(1-isopropyl-4-methyl-1H-pyrazol-5-yl)-7-methyl-4-(4-(1-methyl-4-(trifluoromethyl)-1H-imidazol-2-yl)benzyl)-6,7-dihydropyrazolo[1,5-α]pyrimidin-5(4H)-one

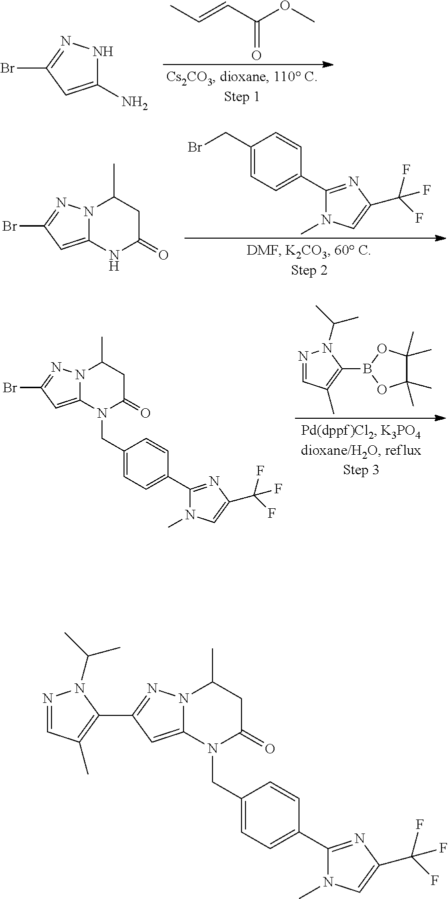

### Step 1. 2-bromo-7-methyl-6,7-dihydropyrazolo[1,5-α]pyrimidin-5(4H)-one

A mixture of 3-bromo-1H-pyrazol-5-amine (500 mg, 3.09 mmol), methyl (E)-but-2-enoate (465 mg, 4.65 mmol), Cs₂CO₃ (100 mg, 0.307 mmol) in dioxane (2 mL) was heated at 110° C. for 30 min. After cooling to ambient temperature, the mixture was concentrated under vacuum and purified by silica gel chromatography (eluting with 10:1 DCM/MeOH) to give 2-bromo-7-methyl-6,7-dihydropyrazolo[1,5-α]pyrimidin-5(4H)-one (611 mg). ¹H NMR (400 MHz, Chloroform-d) δ 8.53 (s, 1H), 5.76 (s, 1H), 4.56-4.46 (m, 1H), 2.99 (dd, J=16.6, 5.7 Hz, 1H), 2.65 (dd, J=16.6, 7.2 Hz, 1H), 1.56 (d, J=6.6 Hz, 3H). MS (ESI) m/z 230.0, 232.0 [M+H]⁺.

### Step 2. 2-bromo-7-methyl-4-(4-(1-methyl-4-(trifluoromethyl)-1H-imidazol-2-yl)benzyl)-6,7-dihydropyrazolo[1,5-α]pyrimidin-5(4H)-one

2-bromo-7-methyl-4-(4-(1-methyl-4-(trifluoromethyl)-1H-imidazol-2-yl)benzyl)-6,7-dihydropyrazolo[1,5-α]pyrimidin-5(4H)-one was prepared from 2-bromo-7-methyl-6,7-dihydropyrazolo[1,5-α]pyrimidin-5(4H)-one according to step 2 of Example 1. MS (ESI) m/z 468.1, 470.1 [M+H]⁺.

### Step 3. 2-(1-isopropyl-4-methyl-1H-pyrazol-5-yl)-7-methyl-4-(4-(1-methyl-4-(trifluoromethyl)-1H-imidazol-2-yl)benzyl)-6,7-dihydropyrazolo[1,5-α]pyrimidin-5(4H)-one

Example 36 2-(1-isopropyl-4-methyl-1H-pyrazol-5-yl)-7-methyl-4-(4-(1-methyl-4-(trifluoromethyl)-1H-imidazol-2-yl)benzyl)-6,7-dihydropyrazolo[1,5-α]pyrimidin-5(4H)-one was synthesized from 2-bromo-7-methyl-4-(4-(1-methyl-4-(trifluoromethyl)-1H-imidazol-2-yl)benzyl)-6,7-dihydropyrazolo[1,5-α]pyrimidin-5(4H)-one according to step 3 of Example 1. ¹H NMR (400 MHz, Chloroform-d) δ 7.61 (d, J=8.0 Hz, 2H), 7.43 (d, J=8.0 Hz, 2H), 7.36 (s, 1H), 7.30 (d, J=1.2 Hz, 1H), 5.78 (s, 1H), 5.13 (d, J=15.2 Hz, 1H), 5.00 (d, J=15.2 Hz, 1H), 4.92-4.87 (m, 1H), 4.62-4.57 (m, 1H), 3.74 (s, 3H), 3.18 (dd, J=16.4, 5.6 Hz, 1H), 2.85 (dd, J=16.4, 7.6 Hz, 1H), 2.02 (s, 3H), 1.60 (d, J=6.4 Hz, 3H), 1.45 (d, J=6.4 Hz, 6H). MS (ESI) m/z 512.2 [M+H]⁺.

### Example 37. 2-(1-isopropyl-4-methyl-1H-pyrazol-5-yl)-4-(4-(1-methyl-4-(trifluoromethyl)-1H-imidazol-2-yl)benzyl)-6,7-dihydro-[1,2,4]triazolo[1,5-α]pyrimidin-5(4H)-one

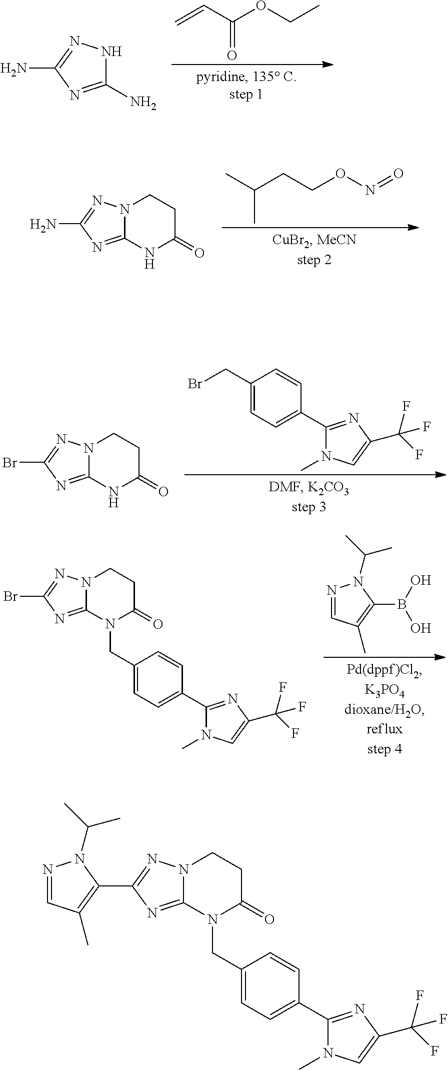

### Step 1. 2-amino-6,7-dihydro-[1,2,4]triazolo[1,5-α]pyrimidin-5(4H)-one

A mixture of 1H-1,2,4-triazole-3,5-diamine (1g, 10.1 mmol), ethyl acrylate (1.52 g, 15.15 mmol) and pyridine (15 mL) was heated at 135° C. in sealed tube for 12 h. Solvent was removed under vacuum. The residue was dissolved in EtOAc and filtered through celite. The filtrate was concentrated to give 2-amino-6,7-dihydro-[1,2,4]triazolo[1,5-α]pyrimidin-5(4H)-one (1.4 g). MS (ESI) m/z 154.1 [M+H]⁺.

### Step 2. 2-bromo-6,7-dihydro-[1,2,4]triazolo[1,5-α]pyrimidin-5(4H)-one

To a solution of 2-amino-6,7-dihydro-[1,2,4]triazolo[1,5-α]pyrimidin-5(4H)-one (1.2 g, 7.84 mmol) in MeCN (30 mL) was added isopentyl nitrite (918 mg, 7.84 mmol) and CuBr₂ (1.75 g, 7.84 mmol). The mixture was heated at 70° C. for 2 h. The mixture was filtered through celite and concentrated under vacuum. The residue was purified by silica gel chromatography (eluting with EtOAc) to give 2-bromo-6,7-dihydro-[1,2,4]triazolo[1,5-α]pyrimidin-5(4H)-one (607 mg). ¹H NMR (400 MHz, DMSO-d₆) δ 6.57 (s, 1H), 4.22 (t, J=7.2 Hz, 2H), 2.87 (t, J=7.2 Hz, 2H). MS (ESI) m/z 217.0, 219.0 [M+H]⁺.

### Step 3. 2-bromo-4-(4-(1-methyl-4-(trifluoromethyl)-1H-imidazol-2-yl)benzyl)-6,7-dihydro-[1,2,4]triazolo[1,5-α]pyrimidin-5(4H)-one

To a solution of 2-bromo-6,7-dihydro-[1,2,4]triazolo[1,5-α]pyrimidin-5(4H)-one (200 mg, 0.92 mmol) in DMF (5 mL) was added 2-(4-(bromomethyl)phenyl)-1-methyl-4-(trifluoromethyl)-1H-imidazole (293 mg, 0.92 mmol) and K₂CO₃ (381 mg, 2.76 mmol). The mixture was stirred at ambient temperature for 12 h then diluted with EtOAc and washed with water and brine. The organic layer was dried with Na₂SO₄, concentrated and purified by silica gel chromatography (eluting with 1/2 EtOAc/PE) to afford 2-bromo-4-(4-(1-methyl-4-(trifluoromethyl)-1H-imidazol-2-yl)benzyl)-6,7-dihydro-[1,2,4]triazolo[1,5-α]pyrimidin-5(4H)-one (103 mg). MS (ESI) m/z 455.1, 457.1 [M+H]⁺.

### Step 4. 2-(1-isopropyl-4-methyl-1H-pyrazol-5-yl)-4-(4-(1-methyl-4-(trifluoromethyl)-1H-imidazol-2-yl)benzyl)-6,7-dihydro-[1,2,4]triazolo[1,5-α]pyrimidin-5(4H)-one

Example 37 2-(1-isopropyl-4-methyl-1H-pyrazol-5-yl)-4-(4-(1-methyl-4-(trifluoromethyl)-1H-imidazol-2-yl)benzyl)-6,7-dihydro-[1,2,4]triazolo[1,5-α]pyrimidin-5(4H)-one was prepared from 2-bromo-4-(4-(1-methyl-4-(trifluoromethyl)-1H-imidazol-2-yl)benzyl)-6,7-dihydro-[1,2,4]triazolo[1,5-α]pyrimidin-5(4H)-one and (1-isopropyl-4-methyl-1H-pyrazol-5-yl)boronic acid according to step 3 of Example 1.

Table 5. The compounds listed in Table 5 were synthesized according to Example 37 using the appropriate commercially available reagents and/or intermediates described above.

TABLE 5

| Example | | | |
|---|---|---|---|
| number | Structure | ¹H NMR (δ ppm) | MS (ESI) |
| 37 | 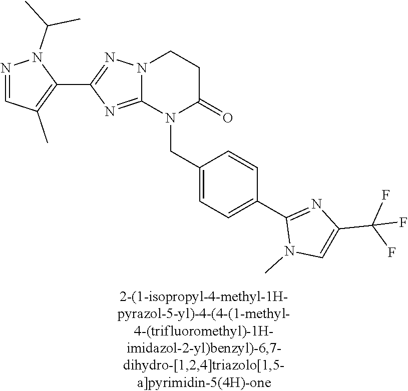 [SMILES: Cc1cnn(C(C)C)c1-c1nc2n(n1)CCC(=O)N2Cc1ccc(-c2nc(C(F)(F)F)cn2C)cc1] | ¹H NMR (400 MHz, Chloroform-d) δ 7.65 (d, J = 8.0 Hz, 2H), 7.59 (d, J = 8.0 Hz, 2H), 7.40 (s, 1H), 7.29 (s, 1H), 5.36-5.29 (m, 1H), 5.19 (s, 2H), 4.39 (t, J = 7.6 Hz, 2H), 3.74 (s, 3H), 3.13 (t, J = 7.2 Hz, 2H), 2.26 (s, 3H), 1.51 (d, J = 6.8 Hz, 6H). | m/z: 499.3 [M + H]⁺ |
| 38 | 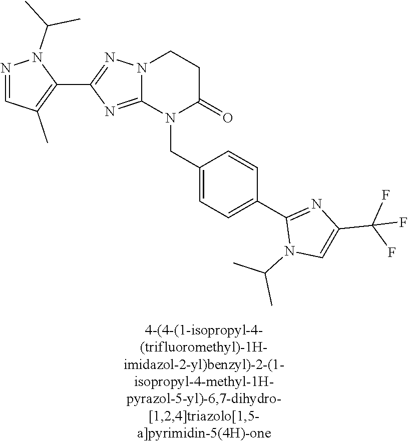 [SMILES: Cc1cnn(C(C)C)c1-c1nc2n(n1)CCC(=O)N2Cc1ccc(-c2nc(C(F)(F)F)cn2C(C)C)cc1] | ¹H NMR (400 MHz, Chloroform-d) δ 7.64 (d, J = 7.6 Hz, 2H), 7.49 (d, J = 7.6 Hz, 2H), 7.39 (s, 2H), 5.36- 5.29 (m, 1H), 5.19 (s, 2H), 4.54-4.51 (m, 1H), 4.36 (s, 2H), 3.10 (s, 2H), 2.26 (s, 3H), 1.49 (d, J = 6.4 Hz, 6H), 1.44 (d, J = 6.4 Hz, 6H). | m/z: 527.3 [M + H]⁺ |
| 39 | 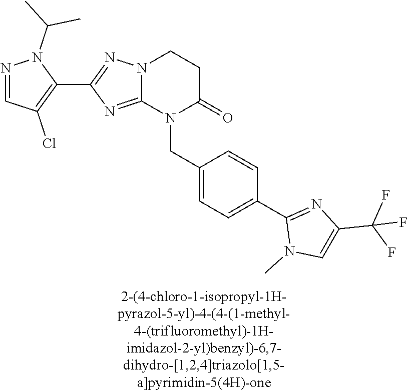 [SMILES: CC(C)n1ncc(Cl)c1-c1nc2n(n1)CCC(=O)N2Cc1ccc(-c2nc(C(F)(F)F)cn2C)cc1] | ¹H NMR (400 MHz, Chloroform-d) δ 7.66 (d, J = 8.3 Hz, 2H), 7.58 (d, J = 8.3 Hz, 2H), 7.54 (s, 1H), 7.31- 7.29 (m, 1H), 5.27-5.18 (m, 3H), 4.41 (t, J = 7.4 Hz, 2H), 3.75 (s, 3H), 3.12 (t, J = 7.4 Hz, 2H), 1.50 (d, J = 6.6 Hz, 6H). | m/z: 519.2 [M + H]⁺ |
| 40 | 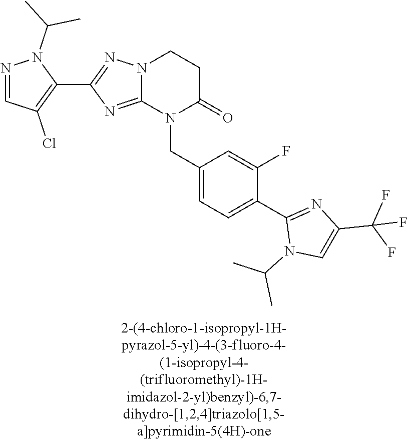 [SMILES: CC(C)n1cc(C(F)(F)F)nc1-c1ccc(CN2C(=O)CCn3nc(-c4c(Cl)cnn4C(C)C)nc32)cc1F] | ¹H NMR (400 MHz, Chloroform-d) δ 7.55-7.40 (m, 5H), 5.26-5.13 (m, 3H), 4.42 (t, J = 7.3 Hz, 2H), 4.27- 4.21 (m, 1H), 3.13 (t, J = 7.3 Hz, 2H), 1.50 (d, J = 6.6 Hz, 6H), 1.40 (d, J = 6.6 Hz, 6H). | m/z: 565.2 [M + H]⁺ |
| 41 | 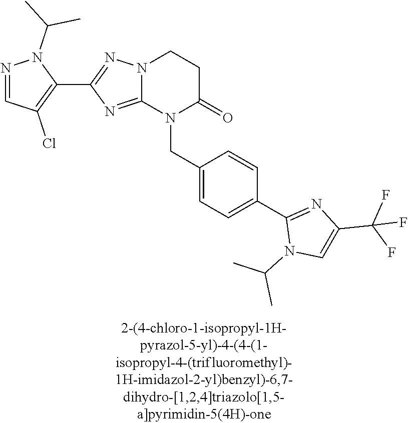 [SMILES: CC(C)n1cc(C(F)(F)F)nc1-c1ccc(CN2C(=O)CCn3nc(-c4c(Cl)cnn4C(C)C)nc32)cc1] | ¹H NMR (400 MHz, Chloroform-d) δ 7.65 (d, J = 8.0 Hz, 2H), 7.54 (s, 1H), 7.49 (d, J = 8.0 Hz, 2H), 7.42-7.39 (m, 1H), 5.24 (q, J = 6.6 Hz, 1H), 5.20 (s, 2H), 4.56-4.49 (m, 1H), 4.40 (t, J = 7.3 Hz, 2H), 3.12 (t, J = 7.3 Hz, 2H), 1.50 (d, J = 6.6 Hz, 6H), 1.44 (d, J = 6.6 Hz, 6H). | m/z: 547.2 [M + H]⁺ |
| 42 | 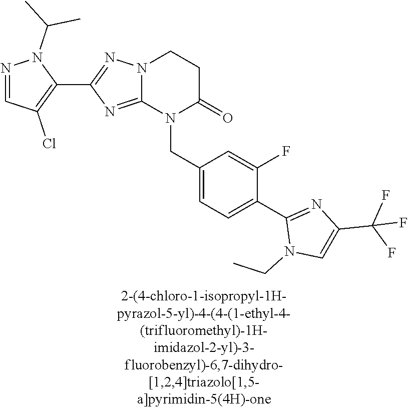 [SMILES: CCn1cc(C(F)(F)F)nc1-c1ccc(CN2C(=O)CCn3nc(-c4c(Cl)cnn4C(C)C)nc32)cc1F] | ¹H NMR (400 MHz, Chloroform-d) δ 7.60-7.49 (m, 2H), 7.49-7.36 (m, 3H), 5.27-5.14 (m, 3H), 4.42 (t, J = 7.3 Hz, 2H), 3.90 (q, J = 7.2 Hz, 2H), 3.13 (t, J = 7.3 Hz, 2H), 1.50 (d, J = 6.6 Hz, 6H), 1.38 (t, J = 7.3 Hz, 3H). | m/z: 551.2 [M + H]⁺ |
| 43 | 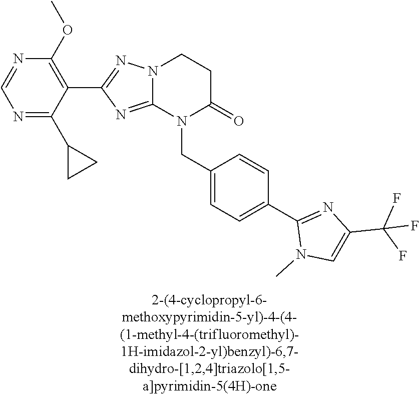 [SMILES: COc1ncnc(C2CC2)c1-c1nc2n(n1)CCC(=O)N2Cc1ccc(-c2nc(C(F)(F)F)cn2C)cc1] | ¹H NMR (400 MHz, Chloroform-d) δ 8.63 (s, 1H), 7.65 (d, J = 8.1 Hz, 2H), 7.56 (d, J = 8.1 Hz, 2H), 7.30 (s, 1H), 5.21 (s, 2H), 4.41 (t, J = 7.3 Hz, 2H), 3.97 (s, 3H), 3.75 (s, 3H), 3.12 (t, J = 7.3 Hz, 2H), 2.05-1.99 (m, 1H), 1.23 (dq, J = 6.5, 4.0 Hz, 2H), 0.95 (dq, J = 6.9, 3.8 Hz, 2H). | m/z: 525.2 [M + H]⁺ |
| 44 | 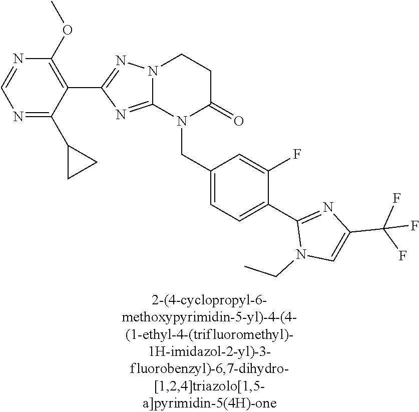 [SMILES: CCn1cc(C(F)(F)F)nc1-c1ccc(CN2C(=O)CCn3nc(-c4c(OC)ncnc4C4CC4)nc32)cc1F] | ¹H NMR (400 MHz, Chloroform-d) δ 8.63 (s, 1H), 7.51 (t, J = 7.4 Hz, 1H), 7.47- 7.38 (m, 3H), 5.19 (s, 2H), 4.42 (t, J = 7.3 Hz, 2H), 3.97 (s, 3H), 3.91 (q, J = 7.3 Hz, 2H), 3.13 (t, J = 7.3 Hz, 2H), 2.07-2.01 (m, 1H), 1.39 (t, J = 7.3 Hz, 3H), 1.23 (dd, J = 7.0, 4.2 Hz, 2H), 0.98 (dq, J = 6.9, 3.7 Hz, 2H). | m/z: 557.2 [M + H]⁺ |
| 45 | 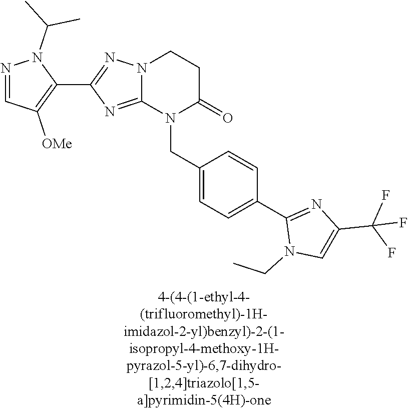 [SMILES: CCn1cc(C(F)(F)F)nc1-c1ccc(CN2C(=O)CCn3nc(-c4c(OC)cnn4C(C)C)nc32)cc1] | ¹H NMR (400 MHz, Chloroform-d) δ 7.60 (d, J = 8.0 Hz, 2H), 7.51 (d, J = 8.0 Hz, 2H), 7.35 (s, 2H), 5.36- 5.28 (m, 1H), 5.18 (s, 2H), 4.38 (t, J = 7.3 Hz, 2H), 4.03 (q, J = 7.3 Hz, 2H), 3.89 (s, 3H), 3.07 (s, 2H), 1.49 (d, J = 6.6 Hz, 6H), 1.41 (t, J = 7.3 Hz, 3H). | m/z: 529.2 [M + H]⁺ |
| 46 | 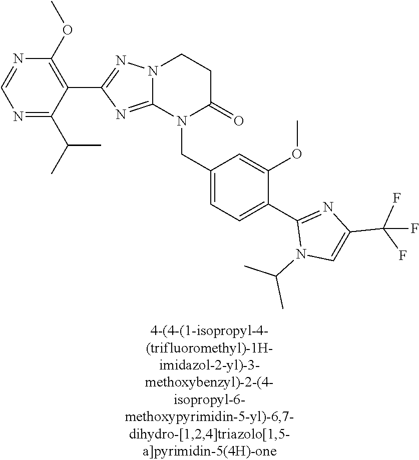 [SMILES: COc1cc(CN2C(=O)CCn3nc(-c4c(OC)ncnc4C(C)C)nc32)ccc1-c1nc(C(F)(F)F)cn1C(C)C] | ¹H NMR (400 MHz, Methanol-d₄) δ 8.74 (s, 1H), 7.84 (s, 1H), 7.30 (d, J = 7.7 Hz, 1H), 7.24 (s, 1H), 7.16 (d, J = 7.7 Hz, 1H), 5.19 (s, 2H), 4.44 (t, J = 7.3 Hz, 2H), 4.16- 4.09 (m, 1H), 3.94 (s, 3H), 3.81 (s, 3H), 3.20 (t, J = 7.3 Hz, 2H), 3.05-2.98 (m, 1H), 1.37 (d, J = 6.5 Hz, 6H), 1.18 (d, J = 6.8 Hz, 6H). | m/z: 585.2 [M + H]⁺ |

### Example 47. 2-(1-isopropyl-4-methyl-1H-pyrazol-5-yl)-4-(4-(1-methyl (trifluoromethyl)-1H-imidazol-2-yl)benzyl)-4,5,6,7-tetrahydropyrazolo[1,5-α]pyrimidine

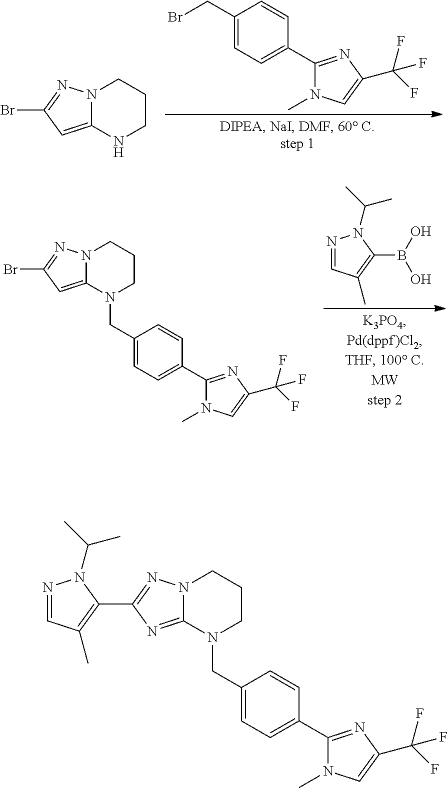 [SMILES: Brc1cc2n(n1)CCCN2 ; Cn1cc(C(F)(F)F)nc1-c1ccc(CBr)cc1 ; Cn1cc(C(F)(F)F)nc1-c1ccc(CN2CCCn3nc(Br)cc32)cc1 ; Cc1cnn(C(C)C)c1B(O)O ; Cc1cnn(C(C)C)c1-c1nc2n(n1)CCCN2Cc1ccc(-c2nc(C(F)(F)F)cn2C)cc1]

### Step 1. 2-bromo-4-(4-(1-methyl-4-(trifluoromethyl)-1H-imidazol-2-yl)benzyl)-4,5,6,7-tetrahydropyrazolo[1,5-α]pyrimidine

2-bromo-4-(4-(1-methyl-4-(trifluoromethyl)-1H-imidazol-2-yl)benzyl)-4,5,6,7-tetrahydropyrazolo[1,5-α]pyrimidine was synthesized form 2-bromo-4,5,6,7-tetrahydropyrazolo[1,5-α]pyrimidine (prepared according to step 1 of Example 24) according to step 5 of Example 24. MS (ESI) m/z 440.1, 442.1 [M+H]⁺.

### Step 2. 2-(1-isopropyl-4-methyl-1H-pyrazol-5-yl)-4-(4-(1-methyl-4-(trifluoromethyl)-1H-imidazol-2-yl)benzyl)-4,5,6,7-tetrahydropyrazolo[1,5-α]pyrimidine

To a solution of 2-bromo-4-(4-(1-methyl-4-(trifluoromethyl)-1H-imidazol-2-yl)benzyl)-4,5,6,7-tetrahydropyrazolo[1,5-α]pyrimidine (261.9 mg, 0.59 mmol) in THF (8 mL) was added (1-isopropyl-4-methyl-1H-pyrazol-5-yl)boronic acid (200 mg, 1.19 mmol), K₃PO₄ (776mg, 3.57 mmol) and Pd(dppf)Cl₂ (87 mg, 0.12 mmol). The resulting solution was heated under an atmosphere of N₂ at 100° C. for 48 h in a sealed tube. The reaction mixture was concentrated and purified by prep-TLC (eluting with 1/20 MeOH/DCM) to afford Example 47 2-(1-isopropyl-4-methyl-1H-pyrazol-5-yl)-4-(4-(1-methyl-4-(trifluoromethyl)-1H-imidazol-2-yl)benzyl)-4,5,6,7-tetrahydropyrazolo[1,5-α]pyrimidine (15.2 mg). ¹H NMR (400 MHz, Methanol-d₄) δ 7.74 (s, 1H), 7.69 (d, J=8.4 Hz, 2H), 7.57 (d, d, J=8.0 Hz, 2H), 7.50 (s, 1H), 4.92 (s, 1H), 4.81-4.85 (m, 1H), 4.58 (s, 2H), 4.19 (t, J=6.0 Hz, 2H), 3.80 (s, 3H), 3.36 (t, J=5.6 Hz, 2H), 2.32-2.29 (m, 2H), 2.06 (s, 3H), 1.43 (d, J=6.8 Hz, 6H). MS (ESI) m/z 484.3 [M+H]⁺.

### Example 48. 2-(4-cyclopropyl-6-methoxypyrimidin-5-yl)-5-methyl-4-(4-(1-methyl-4-(trifluoromethyl)-1H-imidazol-2-yl)benzyl)-4,5,6,7-tetrahydropyrazolo[1,5-α]pyrimidine

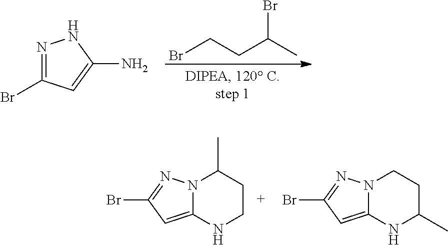

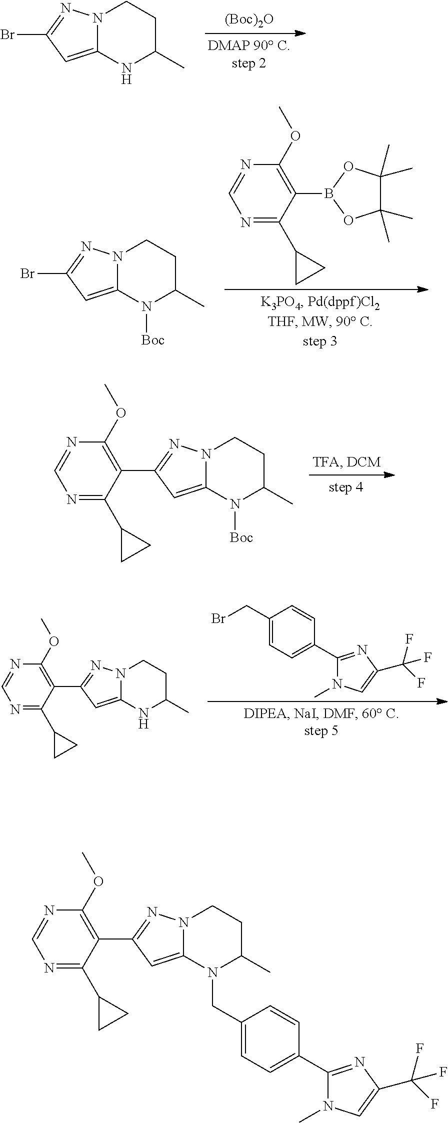

### Step 1. 2-bromo-7-methyl-4,5,6,7-tetrahydropyrazolo[1,5-α]pyrimidine and 2-bromo-5-methyl-4,5,6,7-tetrahydropyrazolo[1,5-α]pyrimidine

To a solution of 3-bromo-1H-pyrazol-5-amine (3.1 g, 19.1 mmol) in dioxane (18 mL) was added 1,3-dibromobutane (4.5 g, 21 mmol) and DIPEA (8.1 g, 62.7 mmol) and the reaction heated at 120° C. for 20 h in a sealed tube. The solvent was removed under vacuum and the crude was purified by Prep-HPLC (condition 2) to afford 2-bromo-7-methyl-4,5,6,7-tetrahydropyrazolo[1,5-α]pyrimidine (2.03 g) and 2-bromo-5-methyl-4,5,6,7-tetrahydropyrazolo-[1,5-α]pyrimidine(503 mg).

2-bromo-7-methyl-4,5,6,7-tetrahydropyrazolo[1,5-α]pyrimidine. ¹H NMR (400 MHz, Chloroform-d) δ (ppm): 5.76 (s, 1H), 5.35 (s, 1H), 4.32-4.26 (m, 1H), 3.38-3.23 (m, 2H), 2.23-2.16 (m, 1H), 1.90-1.82 (m, 1H), 1.51 (d, J=6.4 Hz, 3H).

2-bromo-5-methyl-4,5,6,7-tetrahydropyrazolo[1,5-α]pyrimidine. ¹H NMR (400 MHz, Chloroform-d) δ (ppm): 7.18 (bs, 1H), 5.41 (s, 1H), 4.24-4.19 (m, 1H), 4.08-4.01 (m, 1H), 3.54-3.46 (m, 1H), 2.11-2.05 (m, 1H), 1.92-1.82 (m, 1H), 1.28 (d, J=6.3 Hz, 3H).

### Step 2 to step 5. 2-(4-cyclopropyl-6-methoxypyrimidin-5-yl)-5-methyl-4-(4-(1-methyl-4-(trifluoromethyl)-1H-imidazol-2-yl)benzyl)-4,5,6,7-tetrahydropyrazolo[1,5-α]pyrimidine

Example 48 2-(4-cyclopropyl-6-methoxypyrimidin-5-yl)-5-methyl-4-(4-(1-methyl-4-(trifluoromethyl)-1H-imidazol-2-yl)benzyl)-4,5,6,7-tetrahydropyrazolo[1,5-α]pyrimidine was synthesized from 2-bromo-5-methyl-4,5,6,7-tetrahydropyrazolo[1,5-α]pyrimidine following step 2 to step 5 of Example 24.

Table 6. The compounds listed in Table 6 were synthesized according to Example 48 using the appropriate commercially available reagents and/or intermediates described above.

TABLE 6

| Example | | | |
|---|---|---|---|
| number | Structure | ¹H NMR (δ ppm) | MS (ESI) |
| 48 | 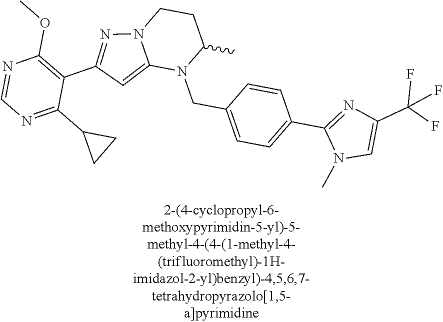 [SMILES: COc1ncnc(C2CC2)c1-c1cc2n(n1)CCC(C)N2Cc1ccc(-c2nc(C(F)(F)F)cn2C)cc1] | ¹H NMR (400 MHz, Chloroform-d) δ 8.53 (s, 1H), 7.61 (d, J = 8.0 Hz, 2H), 7.46 (d, J = 8.0 Hz, 2H), 7.31 (s, 1H), 5.42 (s, 1H), 4.53 (d, J = 16.0 Hz, 1H), 4.36 (d, J = 16.0 Hz, 1H), 4.24-4.19 (m, 2H), 3.92 (s, 3H), 3.77 (s, 3H), 3.49-3.45 (m, 1H), 2.36- 2.27 (m, 2H), 2.06-2.02 (m, 1H), 1.25 (d, J = 6.4 Hz, 3H), 1.15-1.13 (m, 2H), 0.92-0.89 (m, 2H). | m/z: 524.2 [M + H]⁺ |
| 49 | 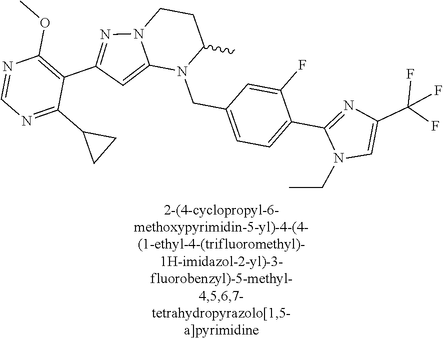 [SMILES: CCn1cc(C(F)(F)F)nc1-c1ccc(CN2c3cc(-c4c(OC)ncnc4C4CC4)nn3CCC2C)cc1F] | ¹H NMR (400 MHz, Methanol-d₄) δ 8.48 (s, 1H), 7.86 (s, 1H), 7.52 (t, J = 7.6 Hz, 1H), 7.36 (dd, J = 15.1, 9.5 Hz, 2H), 5.53 (s, 1H), 4.49 (s, 2H), 4.35 (q, J = 5.8 Hz, 1H), 3.97 (q, J = 7.1 Hz, 2H), 3.90 (s, 3H), 3.37-3.22 (m, 2H), 2.39 (dd, J = 8.6, 5.2 Hz, 1H), 2.16 (s, 1H), 2.03 (d, J = 4.7 Hz, 1H), 1.54 (d, J = 6.4 Hz, 3H), 1.35 (t, J = 7.2 Hz, 3H), 1.12-1.06 (m, 2H), 0.96- 0.87 (m, 2H). | m/z: 556.3 [M + H]⁺ |
| 50 | 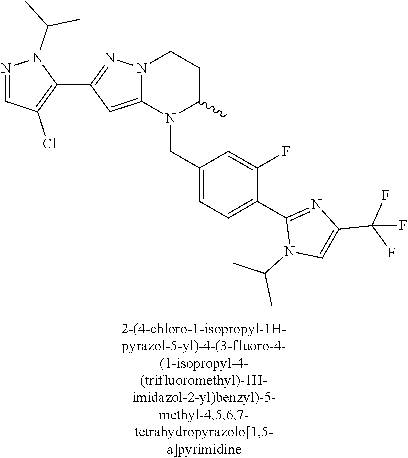 [SMILES: CC1CCn2nc(-c3c(Cl)cnn3C(C)C)cc2N1Cc1ccc(-c2nc(C(F)(F)F)cn2C(C)C)c(F)c1] | ¹H NMR (400 MHz, Chloroform-d) δ 7.54-7.47 (m, 1H), 7.44 (d, J = 4.1 Hz, 2H), 7.27-7.12 (m, 2H), 5.63 (s, 1H), 5.31-5.24 (m, 1H), 4.49 (d, J = 16.4 Hz, 1H), 4.35 (d, J = 16.4 Hz, 1H), 4.31-4.10 (m, 3H), 3.54-3.47 (m, 1H), 2.35-2.27 (m, 1H), 2.10-2.01 (m, 1H), 1.44 (dd, J = 6.6, 2.6 Hz, 6H), 1.41 (d, J = 6.7 Hz, 6H), 1.25 (d, J = 6.4 Hz, 3H). | m/z: 564.2 [M + H]⁺ |

### Example 51. 2-(4-cyclopropyl-6-methoxypyrimidin-5-yl)-7-methyl-4-(4-(1-methyl-4-(trifluoromethyl)-1H-imidazol-2-yl)benzyl)-4,5,6,7-tetrahydropyrazolo[1,5-α]pyrimidine

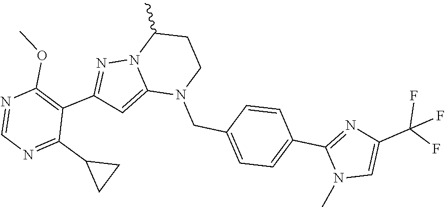 [SMILES: COc1ncnc(C2CC2)c1-c1cc2n(n1)C(C)CCN2Cc1ccc(-c2nc(C(F)(F)F)cn2C)cc1]

Example 51 2-(4-cyclopropyl-6-methoxypyrimidin-5-yl)-7-methyl-4-(4-(1-methyl-4-(trifluoromethyl)-1H-imidazol-2-yl)benzyl)-4,5,6,7-tetrahydropyrazolo[1,5-α]pyrimidine was synthesized from 2-bromo-7-methyl-4,5,6,7-tetrahydropyrazolo[1,5-α]pyrimidine (prepared in step 1 of Example 48) following step 2 to step 5 of Example 24. ¹H NMR (400 MHz, Methanol-d₄) δ 8.60 (s, 1H), 7.67 (m, 6H), 4.59 (s, 2H), 4.47 (m, 1H), 3.96 (s, 3H), 3.79 (s, 3H), 3.35 (m, 2H), 2.41 (m, 1H), 2.12 (m, 2H), 1.57 (d, J=6.4 Hz, 3H), 1.02 (m, 2H), 0.98 (m, 2H). MS (ESI) m/z 524.2 [M+H]⁺.

### Example 52. 2-(1-isopropyl-4-methyl-1H-pyrazol-5-yl)-4-(4-(5-methyl-3-(trifluoromethyl)-1H-pyrazol-1-yl)benzyl)-4,5,6,7-tetrahydropyrazolo[1,5-α]pyrimidine

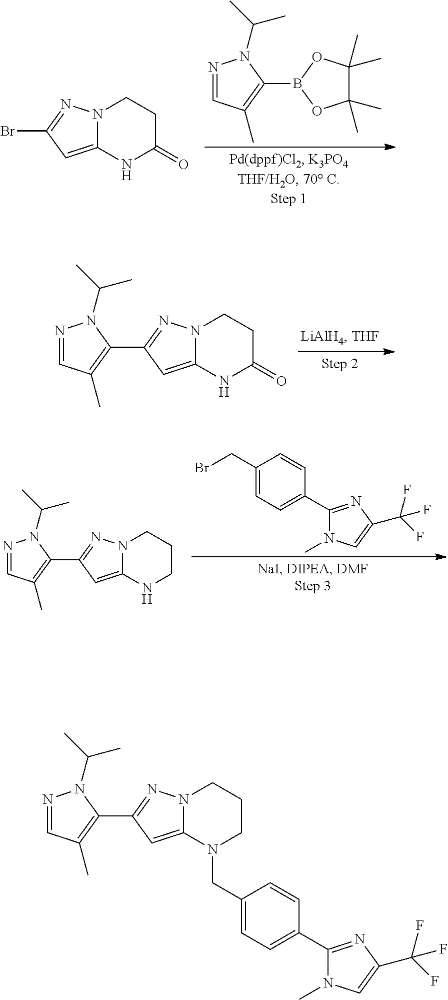 [SMILES: O=C1CCn2nc(Br)cc2N1 ; Cc1cnn(C(C)C)c1B1OC(C)(C)C(C)(C)O1 ; Cc1cnn(C(C)C)c1-c1cc2n(n1)CCC(=O)N2 ; Cc1cnn(C(C)C)c1-c1cc2n(n1)CCCN2 ; Cn1cc(C(F)(F)F)nc1-c1ccc(CBr)cc1 ; Cc1cnn(C(C)C)c1-c1cc2n(n1)CCCN2Cc1ccc(-c2nc(C(F)(F)F)cn2C)cc1]

### Step 1. 2-(1-isopropyl-4-methyl-1H-pyrazol-5-yl)-6,7-dihydropyrazolo[1,5-α]pyrimidin-5(4H)-one

To a solution of 2-bromo-6,7-dihydropyrazolo[1,5-α]pyrimidin-5(4H)-one (2 mg, 9.3 mmol) in dioxane/H₂O (22 mL, 10/1, v/v) was added K₃PO₄ (4.03 g, 18.6 mmol), 1-isopropyl-4-methyl-5-(4,4,5,5-tetramethyl-1,3,2-dioxaborolan-2-yl)-1H-pyrazole (4.65 g, 18.6 mmol) and Pd(dppf)Cl₂ (340 mg, 0.46 mmol) at ambient temperature. The mixture was heated under an atmosphere of N₂ at 70° C. for 12 h. After cooling to ambient temperature, the mixture was filtered through celite and washed with EtOAc (20 mL). The filtrate was concentrated and purified by column chromatography on silica gel (eluting with 7/3 PE/EtOAc) to give 2-(1-isopropyl-4-methyl-1H-pyrazol-5-yl)-6,7-dihydropyrazolo[1,5-α]pyrimidin-5(4H)-one (1.1 g). MS (ESI) m/z 260.2 [M+H]⁺.

### Step 2. 2-(1-isopropyl-4-methyl-1H-pyrazol-5-yl)-4,5,6,7-tetrahydropyrazolo[1,5-α]pyrimidine

To a solution of 2-(1-isopropyl-4-methyl-1H-pyrazol-5-yl)-6,7-dihydropyrazolo[1,5-α]pyrimidin-5(4H)-one (1.1 g 4.24 mmol) in dry THF (30 mL) was added LiAlH₄ (976 mg, 25.4 mmol) at 0° C. The reaction was stirred 0° C. for 30 min then quenched with aqueous NaOH (2M, 1 mL), filtered and washed with THF (20 mL). The combined filtrates were concentrated and purified on a Biotage Isolera One (C₁₈ column, eluting with 10% to 95% MeCN/H₂O) to give 2-(1-isopropyl-4-methyl-1H-pyrazol-5-yl)-6,7-dihydropyrazolo[1,5-α]pyrimidine (800 mg). MS (ESI) m/z 244.2 [M+H]⁺.

### Step 3. 2-(1-isopropyl-4-methyl-1H-pyrazol-5-yl)-4-(4-(5-methyl-3-(trifluoromethyl)-1H-pyrazol-1-yl)benzyl)-4,5,6,7-tetrahydropyrazolo[1,5-α]pyrimidine

To a mixture of 1-(4-(bromomethyl)phenyl)-5-methyl-3-(trifluoromethyl)-1H-pyrazole (94 mg, 0.29 mmol) and 2-(1-isopropyl-4-methyl-1H-pyrazol-5-yl)-4,5,6,7-tetrahydropyrazolo[1,5-α]pyrimidine (60 mg, 0.24 mmol) in DMF (3 mL) was added NaI (73 mg, 0.49 mmol) and DIPEA (63 mg, 0.49 mmol) at ambient temperature. The resulting solution was heated at 40° C.-60° C. for 12 h. The reaction mixture was diluted with EtOAc (30 mL), washed with water and brine. The organic phase was dried with Na₂SO₄, concentrated and purified by prep-HPLC (condition 2) to give Example 52 2-(4-chloro-1-isopropyl-1H-pyrazol-5-yl)-4-(4-(5-methyl-3-(trifluoromethyl)-1H-pyrazol-1-yl)benzyl)-4,5,6,7-tetrahydropyrazolo[1,5-α]pyrimidine (31 mg).

Table 7. The compounds listed in Table 7 were synthesized according to Example 52 using the appropriate commercially available reagents and/or intermediates described above. Enantiomers, when generated, were separated by chiral HPLC and absolute stereochemistries were arbitrarily assigned.

TABLE 7

| Example | | | |
|---|---|---|---|
| number | Structure | ¹H NMR (δ ppm) | MS (ESI) |
| 52 | 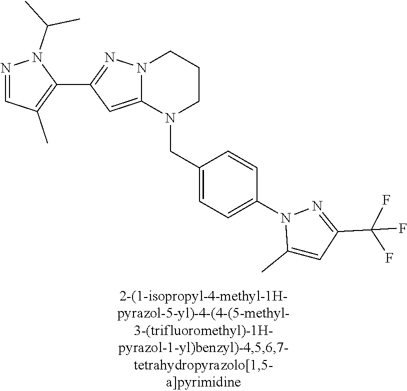 [SMILES: Cc1cnn(C(C)C)c1-c1cc2n(n1)CCCN2Cc1ccc(-n2nc(C(F)(F)F)cc2C)cc1] | ¹H NMR (400 MHz, Chloroform-d) δ 7.52-7.42 (m, 5H), 6.46 (s, 1H), 5.48 (s, 1H), 5.05-5.01 (m, 1H), 4.40 (s, 2H), 4.19 (t, J = 6.0 Hz, 2H), 3.20 (t, J = 6.0 Hz, 2H), 2.35 (s, 3H), 2.28-2.23 (m, 2H), 2.08 (s, 3H), 1.50 (d, J = 6.4 Hz, 6H). | m/z: 484.2 [M + H]⁺ |
| 53 | 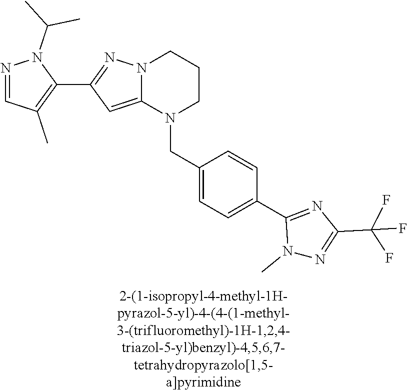 [SMILES: Cc1cnn(C(C)C)c1-c1cc2n(n1)CCCN2Cc1ccc(-c2nc(C(F)(F)F)nn2C)cc1] | ¹H NMR (400 MHz, Chloroform-d) δ 7.71 (d, J = 8.4 Hz, 2H), 7.53-7.51 (m, 2H), 7.38 (s, 1H), 5.44 (s, 1H), 5.02-4.96 (m, 1H), 4.42 (s, 2H), 4.19 (t, J = 6.4 Hz 2H), 4.06 (s, 3H), 3.22 (t, J = 5.6 Hz, 2H), 2.30-2.24 (m, 2H), 2.07 (s, 3H), 1.46 (d, J = 6.4 Hz, 6H). | m/z: 485.2 [M + H]⁺ |
| 54 | 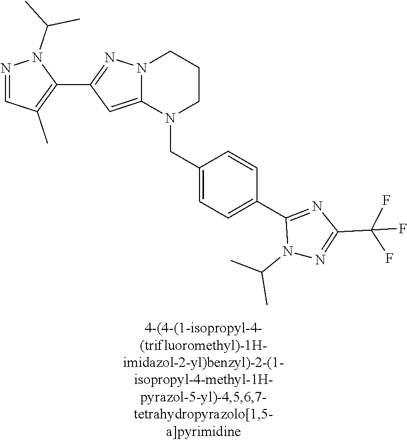 [SMILES: Cc1cnn(C(C)C)c1-c1cc2n(n1)CCCN2Cc1ccc(-c2nc(C(F)(F)F)nn2C(C)C)cc1] | ¹H NMR (400 MHz, Chloroform-d) δ 7.52 (d, J = 7.6 Hz, 2H), 7.44 (d, J = 8 Hz, 4H), 5.47 (s, 1H), 4.99 (s, 1H), 4.55 (m, 1H), 4.40 (s, 2H), 4.18 (s, 2H), 3.20 (s, 2H), 2.25 (s, 2H), 2.07 (s, 3H), 1.46 (s, 6H), 1.44 (s, 6H). | m/z: 512.3 [M + H]⁺ |
| 55 | 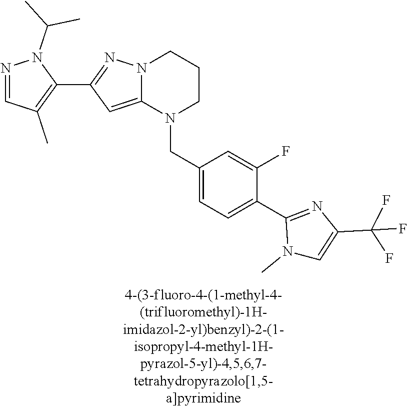 [SMILES: Cc1cnn(C(C)C)c1-c1cc2n(n1)CCCN2Cc1ccc(-c2nc(C(F)(F)F)cn2C)c(F)c1] | ¹H NMR (400 MHz, Chloroform-d) δ 7.60 (t, J = 7.6 Hz, 1H), 7.35 (s, 2H), 7.26 (d, J = 8.8 Hz, 1H), 7.20 (d, J = 10.8 Hz, 1H), 5.42 (s, 1H), 4.99-4.94 (m, 1H), 4.38 (s, 2H), 4.18 (t, J = 6.0 Hz, 2H), 3.65 (d, J = 2.0 Hz, 3H), 3.20 (t, J = 5.6 Hz, 2H), 2.27 (t, J = 5.2 Hz, 2H), 2.06 (s, 3H), 1.45 (d, J = 6.8 Hz, 6H). | m/z: 502.2 [M + H]⁺ |
| 56 | 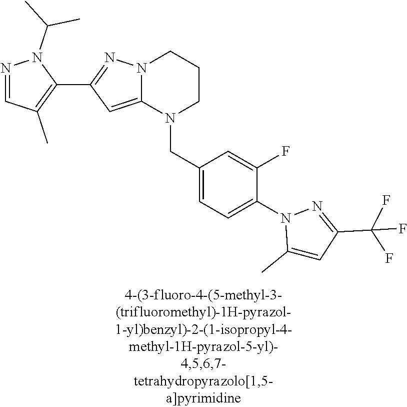 [SMILES: Cc1cnn(C(C)C)c1-c1cc2n(n1)CCCN2Cc1ccc(-n2nc(C(F)(F)F)cc2C)c(F)c1] | ¹H NMR (400 MHz, Chloroform-d) δ 7.46 (t, J = 8.0 Hz, 1H), 7.36 (s, 1H), 7.28 (d, J = 6.8 Hz, 2H), 6.46 (s, 1H), 5.42 (s, 1H), 5.00-4.93 (m, 1H), 4.39 (s, 2H), 4.19 (t, J = 6.4 Hz, 2H), 3.21 (t, J = 5.6 Hz, 2H), 2.30-2.25 (m, 5H), 2.06 (s, 3H), 1.45 (d, J = 6.4 Hz, 6H) | m/z: 502.3 [M + H]⁺ |
| 57 | 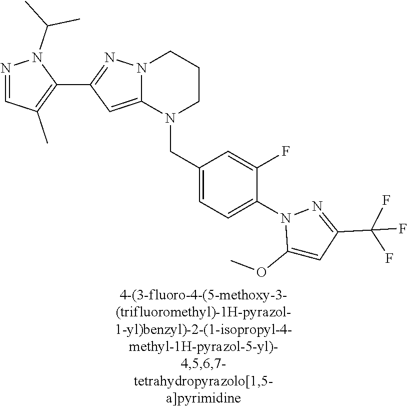 [SMILES: COc1cc(C(F)(F)F)nn1-c1ccc(CN2CCCn3nc(-c4c(C)cnn4C(C)C)cc32)cc1F] | ¹H NMR (400 MHz, Chloroform-d) δ 7.46 (t, J = 7.6 Hz, 1H), 7.36 (s, 1H), 7.24 (d, J = 8.8 Hz, 2H), 5.94 (s, 1H), 5.42 (s, 1H), 5.00-4.93 (m, 1H), 4.37 (s, 2H), 4.18 (t, J = 6.0 Hz, 2H), 3.95 (s, 3H), 3.19 (t, J = 5.6 Hz, 2H), 2.29- 2.25 (m, 2H), 2.07 (s, 3H), 1.46 (d, J = 6.4 Hz, 6H). | m/z: 518.2 [M + H]⁺ |
| 58 | 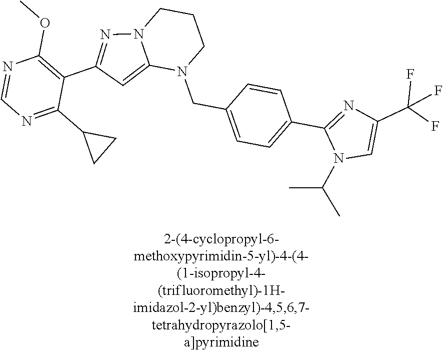 [SMILES: COc1ncnc(C2CC2)c1-c1cc2n(n1)CCCN2Cc1ccc(-c2nc(C(F)(F)F)cn2C(C)C)cc1] | ¹H NMR (400 MHz, Chloroform-d) δ 8.55 (s, 1H), 7.52 (d, J = 4.0 Hz, 2H), 7.46 (d, J = 4.0 Hz, 2H), 7.42 (s, 1H), 5.56 (s, 1H), 4.62-4.52 (m, 1H), 4.39 (s, 2H), 4.20 (q, J = 6.0 Hz, 2H), 3.95 (s, 3H), 3.17 (d, J = 5.6 Hz, 2H), 2.36- 2.32 (m, 1H), 2.26-2.23 (m, 2H), 1.46 (d, J = 6.4 Hz, 6H), 1.18-1.15 (m, 2H), 0.95-0.92 (m, 2H). | m/z: 538.3 [M + H]⁺ |
| 59 | 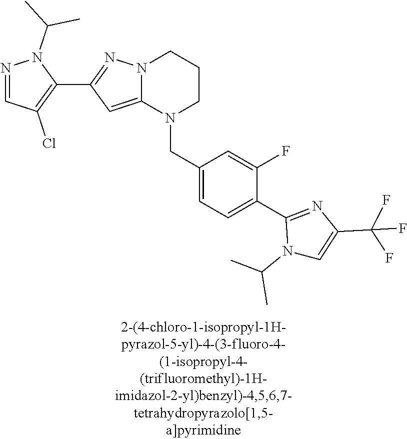 [SMILES: CC(C)n1cc(C(F)(F)F)nc1-c1ccc(CN2CCCn3nc(-c4c(Cl)cnn4C(C)C)cc32)cc1F] | ¹H NMR (400 MHz, Chloroform-d) δ 7.54 (t, J = 7.1 Hz, 1H), 7.47 (s, 1H), 7.44 (s, 1H), 7.24 (s, 1H), 7.18 (d, J = 10.2 Hz, 1H), 5.76 (s, 1H), 5.30-5.20 (m, 1H), 4.40 (s, 2H), 4.33-4.23 (m, 1H), 4.19 (s, 2H), 3.21 (s, 2H), 2.28 (s, 2H), 1.46 (d, J = 6.3 Hz, 6H), 1.42 (d, J = 6.5 Hz, 6H). | m/z: 550.2 [M + H]⁺ |
| 60 | 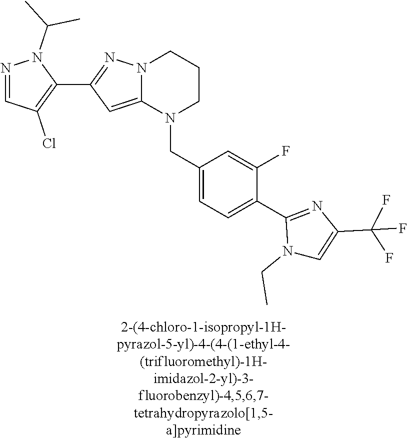 [SMILES: CCn1cc(C(F)(F)F)nc1-c1ccc(CN2CCCn3nc(-c4c(Cl)cnn4C(C)C)cc32)cc1F] | ¹H NMR (400 MHz, Chloroform-d) δ 7.56 (t, J = 7.5 Hz, 1H), 7.47 (s, 1H), 7.41 (q, J = 1.2 Hz, 1H), 7.26-7.23 (m, 1H), 7.18 (dd, J = 10.6, 1.5 Hz, 1H), 5.76 (s, 1H), 5.28-5.22 (m, 1H), 4.40 (s, 2H), 4.19 (t, J = 6.2 Hz, 2H), 4.00-3.88 (m, 2H), 3.25-3.15 (m, 2H), 2.27 (m, 2H), 1.46 (d, J = 6.6 Hz, 6H), 1.40 (t, J = 7.3 Hz, 3H). | m/z: 536.2 [M + H]⁺ |
| 61 | 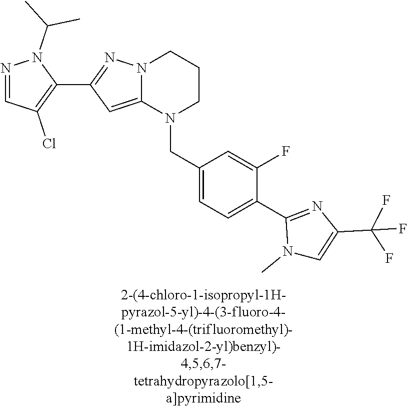 [SMILES: CC(C)n1ncc(Cl)c1-c1cc2n(n1)CCCN2Cc1ccc(-c2nc(C(F)(F)F)cn2C)c(F)c1] | ¹H NMR (400 MHz, Chloroform-d) δ 7.62 (t, J = 7.5 Hz, 1H), 7.48 (s, 1H), 7.38 (s, 1H), 7.28 (s, 1H), 7.21 (d, J = 10.7 Hz, 1H), 5.77 (s, 1H), 5.31-5.22 (m, 1H), 4.41 (s, 2H), 4.20 (t, J = 6.1 Hz, 2H), 3.67 (d, J = 1.9 Hz, 3H), 3.27- 3.06 (m, 2H), 2.37-2.21 (m, 2H), 1.47 (d, J = 6.6 Hz, 6H). | m/z: 522.2 [M + H]⁺ |
| 62 | 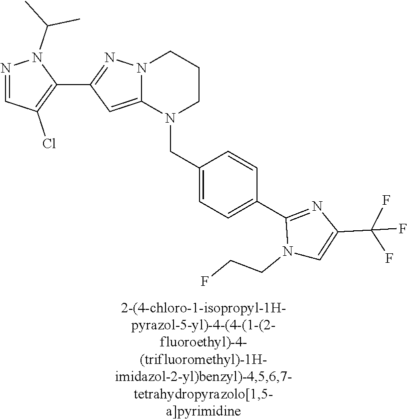 [SMILES: CC(C)n1ncc(Cl)c1-c1cc2n(n1)CCCN2Cc1ccc(-c2nc(C(F)(F)F)cn2CCF)cc1] | ¹H NMR (400 MHz, Chloroform-d) δ 7.53 (d, J = 7.9 Hz, 2H), 7.49-7.40 (m, 4H), 5.75 (s, 1H), 5.27-5.21 (m, 1H), 4.71 (t, J = 4.7 Hz, 1H), 4.59 (t, J = 4.7 Hz, 1H), 4.38 (s, 2H), 4.33 (t, J = 4.8 Hz, 1H), 4.26 (t, J = 4.7 Hz, 1H), 4.15 (t, J = 6.2 Hz, 2H), 3.19-3.11 (m, 2H), 2.23 (m, 2H), 1.44 (d, J = 6.6 Hz, 6H). | m/z: 536.2 [M + H]⁺ |
| 63 | 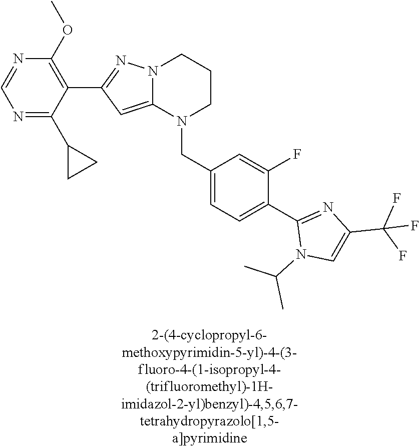 [SMILES: COc1ncnc(C2CC2)c1-c1cc2n(n1)CCCN2Cc1ccc(-c2nc(C(F)(F)F)cn2C(C)C)c(F)c1] | ¹H NMR (400 MHz, Chloroform-d) δ 8.54 (s, 1H), 7.53 (t, J = 7.5 Hz, 1H), 7.44 (s, 1H), 7.26-7.14 (m, 2H), 5.52 (s, 1H), 4.38 (s, 2H), 4.31-4.24 (m, 1H), 4.20 (t, J = 6.2 Hz, 2H), 3.94 (s, 3H), 3.24-3.11 (m, 2H), 2.36-2.24 (m, 3H), 1.42 (d, J = 6.6 Hz, 6H), 1.20-1.12 (m, 2H), 0.93 (dq, J = 7.2, 3.7 Hz, 2H). | m/z: 556.2 [M + H]⁺ |
| 64 | 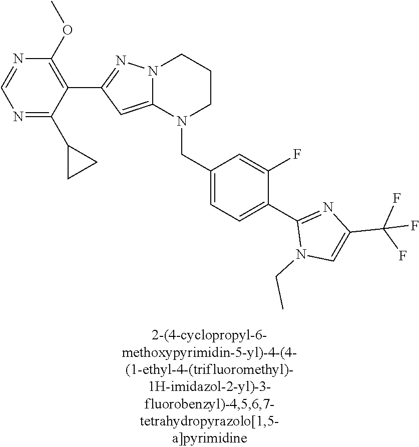 [SMILES: CCn1cc(C(F)(F)F)nc1-c1ccc(CN2CCCn3nc(-c4c(OC)ncnc4C4CC4)cc32)cc1F] | ¹H NMR (400 MHz, Chloroform-d) δ 8.54 (s, 1H), 7.55 (t, J = 7.5 Hz, 1H), 7.41 (d, J = 1.4 Hz, 1H), 7.26-7.13 (m, 2H), 5.51 (s, 1H), 4.38 (s, 2H), 4.20 (t, J = 6.2 Hz, 2H), 3.93 (d, J = 5.4 Hz, 5H), 3.18 (t, J = 5.5 Hz, 2H), 2.36-2.24 (m, 3H), 1.40 (t, J = 7.3 Hz, 3H), 1.16 (dt, J = 6.4, 3.2 Hz, 2H), 0.94 (dt, J = 8.2, 3.3 Hz, 2H). | m/z: 542.2 [M + H]⁺ |
| 65 | 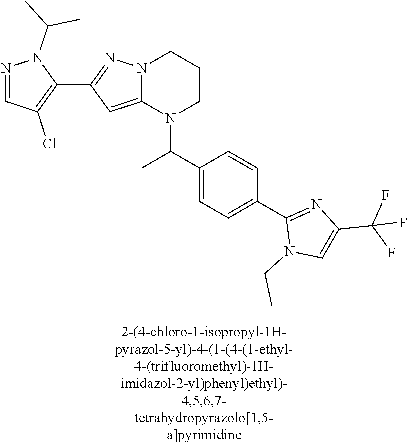 [SMILES: CCn1cc(C(F)(F)F)nc1-c1ccc(C(C)N2CCCn3nc(-c4c(Cl)cnn4C(C)C)cc32)cc1] | ¹H NMR (400 MHz, Chloroform-d) δ 7.58 (d, J = 8.2 Hz, 2H), 7.53-7.46 (m, 3H), 7.37 (s, 1H), 5.80 (s, 1H), 5.31-5.21 (m, 1H), 4.84 (q, J = 6.9 Hz, 1H), 4.20-4.02 (m, 4H), 3.16-3.10 (m, 1H), 2.98-2.92 (m, 1H), 2.18-2.12 (m, 2H), 1.64 (d, J = 7.0 Hz, 3H), 1.45 (t, J = 7.2 Hz, 9H). | m/z: 532.2 [M + H]⁺ |
| 66 | 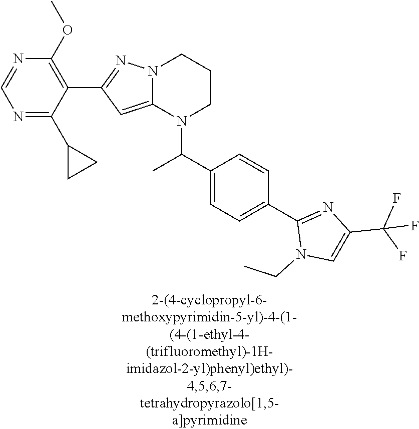 [SMILES: CCn1cc(C(F)(F)F)nc1-c1ccc(C(C)N2CCCn3nc(-c4c(OC)ncnc4C4CC4)cc32)cc1] | ¹H NMR (400 MHz, Chloroform-d) δ 8.54 (s, 1H), 7.58 (d, J = 8.1 Hz, 2H), 7.51 (d, J = 8.2 Hz, 2H), 7.37 (s, 1H), 5.55 (s, 1H), 4.83 (q, J = 6.9 Hz, 1H), 4.20-4.06 (m, 4H), 3.94 (s, 3H), 3.16-3.10 (m, 1H), 2.97-2.91 (m, 1H), 2.39-2.33 (m, 1H), 2.20-2.10 (m, 2H), 1.63 (d, J = 6.9 Hz, 3H), 1.45 (t, J = 7.3 Hz, 3H), 1.19-1.12 (m, 2H), 0.93 (dd, J = 7.9, 2.8 Hz, 2H). | m/z: 538.2 [M + H]⁺ |
| 67 | 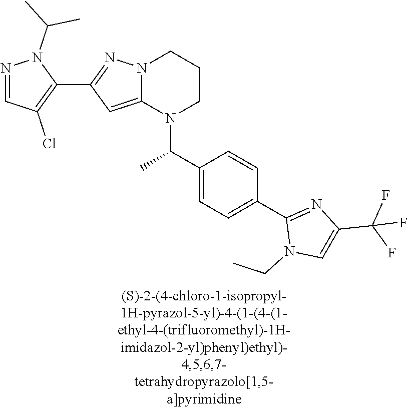 [SMILES: CCn1cc(C(F)(F)F)nc1-c1ccc([C@H](C)N2CCCn3nc(-c4c(Cl)cnn4C(C)C)cc32)cc1] | ¹H NMR (400 MHz, Chloroform-d) δ 7.58 (d, J = 8.3 Hz, 2H), 7.50 (d, J = 8.2 Hz, 2H), 7.47 (s, 1H), 7.39- 7.36 (m, 1H), 5.80 (s, 1H), 5.27 (m, 1H), 4.84 (q, J = 6.9 Hz, 1H), 4.19-4.04 (m, 4H), 3.17-3.08 (m, 1H), 3.00-2.90 (m, 1H), 2.22-2.10 (m, 2H), 1.64 (d, J = 7.0 Hz, 3H), 1.44 (d, J = 7.3 Hz, 9H). | m/z: 532.2 [M + H]⁺ |
| 68 | 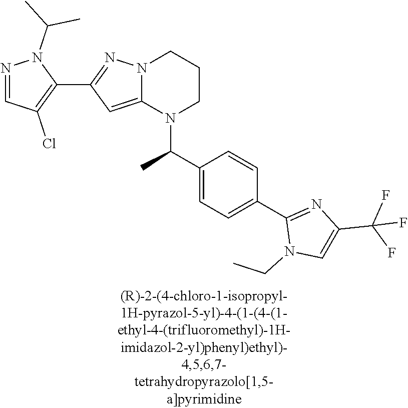 [SMILES: CCn1cc(C(F)(F)F)nc1-c1ccc([C@@H](C)N2CCCn3nc(-c4c(Cl)cnn4C(C)C)cc32)cc1] | ¹H NMR (400 MHz, Chloroform-d) δ 7.58 (d, J = 8.3 Hz, 2H), 7.50 (d, J = 8.2 Hz, 2H), 7.47 (s, 1H), 7.39- 7.36 (m, 1H), 5.80 (s, 1H), 5.27 (m, 1H), 4.84 (d, J = 6.9 Hz, 1H), 4.20-4.03 (m, 4H), 3.17-3.08 (m, 1H), 3.00-2.90 (m, 1H), 2.22-2.10 (m, 2H), 1.64 (d, J = 7.0 Hz, 3H), 1.44 (d, J = 7.3 Hz, 9H). | m/z: 532.2 [M + H]⁺ |

### Example 69. 2-(4-cyclopropyl-6-methoxypyrimidin-5-yl)-4-(4-(4-(trifluoromethyl)-1H-imidazol-2-yl)benzyl)-6,7-dihydropyrazolo[1,5-α]pyrimidin-5(4H)-one

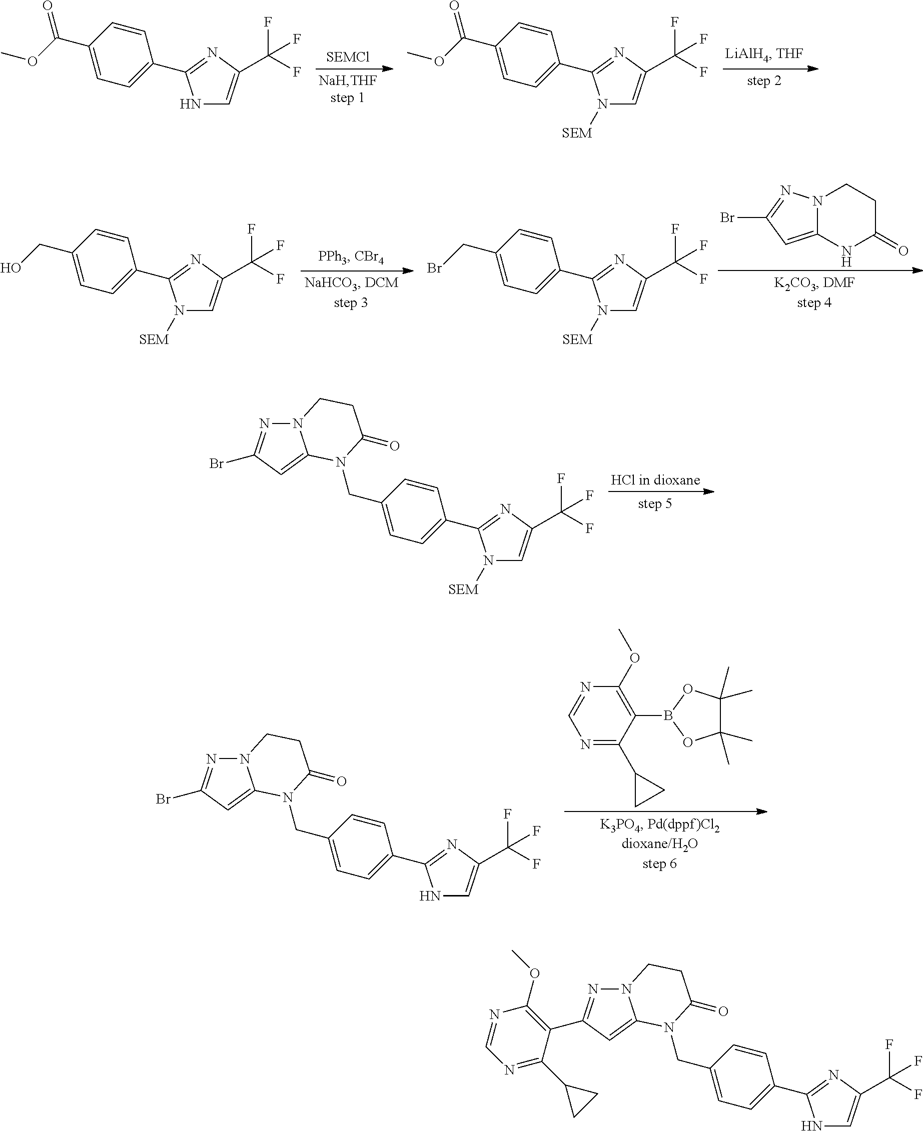

### Step 1. methyl 4-(4-(trifluoromethyl)-1-((2-(trimethylsilyl)ethoxy)methyl)-1H-imidazol-2-yl)benzoate

To a solution of methyl 4-(4-(trifluoromethyl)-1H-imidazol-2-yl)benzoate (7.0 g, 25.9 mmol) in anhydrous THF (40 mL) was added 60% NaH (1.14 g, 60% in mineral oil, 28.5 mmol) at 0° C. The reaction was stirred at 0° C. for 15 min before SEMCl (6 mL, 33.7 mmol) was added. The resulting mixture was warmed to room temperature and stirred for another 15 min then quenched with H₂O (100 mL) and extracted with EtOAc (3×100 mL). The combined organic phases were dried over anhydrous Na₂SO₄, concentrated under vacuum and purified by silica gel chromatography (eluting with 1/40 EtOAc/PE) to afford methyl 4-(4-(trifluoromethyl)-1-((2-(trimethylsilyl)ethoxy)methyl)-1H-imidazol-2-yl)benzoate (8.4 g). MS (ESI) m/z 401.3 [M+H]⁺.

### Step 2. (4-(4-(trifluoromethyl)-1-((2-(trimethylsilyl)ethoxy)methyl)-1H-imidazol-2-yl)phenyl)methanol

To a solution of methyl 4-(4-(trifluoromethyl)-1-((2-(trimethylsilyl)ethoxy)methyl)-1H-imidazol-2-yl)benzoate (7.77 g, 19.4 mmol) in anhydrous THF (60 mL) was added LiAlH₄ (1.47 g, 38.8 mmol) at 0° C. The reaction was stirred at room temperature for 1 h then quenched with aqueous NaOH (2M). The resulting mixture was diluted with DCM (100 mL) and filtered through celite. The filtrate was concentrated under vacuum and purified by silica gel chromatography (eluting with 1/40 MeOH/DCM) to afford (4-(4-(trifluoromethyl)-1-((2-(trimethylsilyl)ethoxy)methyl)-1H-imidazol-2-yl)phenyl)methanol (5.9 g). MS (ESI) m/z 373.3 [M+H]⁺.

### Step 3. 2-(4-(bromomethyl)phenyl)-4-(trifluoromethyl)-1-((2-(trimethylsilyl)ethoxy)methyl)-1H-imidazole

To a solution of (4-(4-(trifluoromethyl)-1-((2-(trimethylsilyl)ethoxy)methyl)-1H-imidazol-2-yl)phenyl)methanol (3.8 g, 10.2 mmol), NaHCO₃ (1.71 g, 20.4 mmol) and PPh₃ (5.36 g, 20.4 mmol) in DCM (70 mL) was added CBr₄ (6.76 g, 20.4 mmol) at room temperature. The resulting mixture was stirred at room temperature for 1 h then filtered through celite and the filtrate concentrated. The residue obtained was purified by silica gel chromatography (eluting with EtOAc/PE 1/10) to afford 2-(4-(bromomethyl)phenyl)-4-(trifluoromethyl)-1-((2-(trimethylsilyl)ethoxy)methyl)-1H-imidazole (3.71 g). MS (ESI) m/z 435.3, 437.3 [M+H]⁺.

### Step 4. 2-bromo-4-(4-(4-(trifluoromethyl)-1-((2-(trimethylsilyl)ethoxy)methyl)-1H-imidazol-2-yl)benzyl)-6,7-dihydropyrazolo[1,5-α]pyrimidin-5(4H)-one

2-bromo-4-(4-(4-(trifluoromethyl)-1-((2-(trimethylsilyl)ethoxy)methyl)-1H-imidazol-2-yl)benzyl)-6,7-dihydropyrazolo[1,5-α]pyrimidin-5(4H)-one was synthesized from 2-(4-(bromomethyl)phenyl)-4-(trifluoromethyl)-1-((2-(trimethylsilyl)ethoxy)methyl)-1H-imidazole according to step 2 of Example 1. MS (ESI) m/z 570.3, 572.3 [M+H]⁺.

### Step 5: 2-bromo-4-(4-(4-(trifluoromethyl)-1H-imidazol-2-yl)benzyl)-6,7-dihydropyrazolo[1,5-α]pyrimidin-5(4H)-one

A mixture of 2-bromo-4-(4-(4-(trifluoromethyl)-1-((2-(trimethylsilyl)ethoxy)methyl)-1H-imidazol-2-yl)benzyl)-6,7-dihydropyrazolo[1,5-α]pyrimidin-5(4H)-one (2.37 g, 4.16 mmol) in HCl dioxane solution (4 M, 100 mL) was heated at 60° C. for 2 h. The solvent was removed in vacuo and the crude residue obtained was dissolved in EtOAc and washed with saturated Na₂CO₃ solution. The organic layer was dried over anhydrous Na₂SO₄, concentrated under vacuum and purified by silica gel chromatography (eluting with MeOH/DCM 1/80) to afford 2-bromo-4-(4-(4-(trifluoromethyl)-1H-imidazol-2-yl)benzyl)-6,7-dihydropyrazolo[1,5-α]pyrimidin-5(4H)-one (1.15 g). MS (ESI) m/z 440.3, 442.3 [M+H]⁺.

### Step 6. 2-(4-cyclopropyl-6-methoxypyrimidin-5-yl)-4-(4-(4-(trifluoromethyl)-1H-imidazol-2-yl)benzyl)-6,7-dihydropyrazolo[1,5-α]pyrimidin-5(4H)-one

Example 69 2-(4-cyclopropyl-6-methoxypyrimidin-5-yl)-4-(4-(4-(trifluoromethyl)-1H-imidazol-2-yl)benzyl)-6,7-dihydropyrazolo[1,5-α]pyrimidin-5(4H)-one was prepared from 2-bromo-4-(4-(4-(trifluoromethyl)-1H-imidazol-2-yl)benzyl)-6,7-dihydropyrazolo[1,5-α]pyrimidin-5(4H)-one according to step 3 of Example 1.

Table 8. The compounds listed in Table 8 were synthesized according to Example 69 using the appropriate commercially available reagents and/or intermediates described above.

TABLE 8

| Example | | | |
|---|---|---|---|
| number | Structure | ¹H NMR (δ ppm) | MS (ESI) |
| 69 | 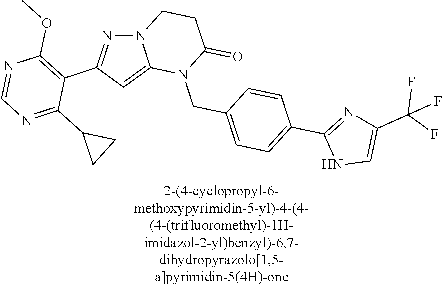 [SMILES: COc1ncnc(C2CC2)c1-c1cc2n(n1)CCC(=O)N2Cc1ccc(-c2nc(C(F)(F)F)c[nH]2)cc1] | ¹H NMR (400 MHz, DMSO- d₆) δ 13.17 (s, 1H), 8.55 (s, 1H), 7.94-7.90 (m, 3H), 7.48 (d, J = 8.0 Hz, 2H), 6.05 (s, 1H), 4.98 (s, 2H), 4.39 (t, J = 7.2 Hz, 2H), 3.81 (s, 3H), 3.08 (t, J = 7.2 Hz, 2H), 2.26-2.20 (m, 1H), 0.99-0.98 (m, 2H), 0.89-0.85 (m, 2H). | m/z: 510.3 [M + H]⁺ |
| 70 | 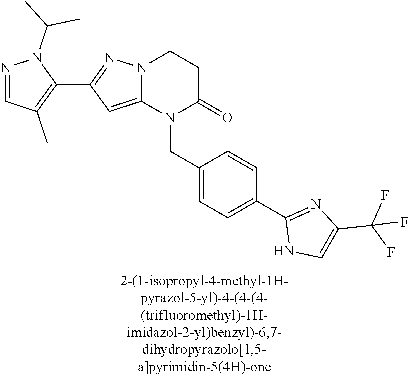 [SMILES: Cc1cnn(C(C)C)c1-c1cc2n(n1)CCC(=O)N2Cc1ccc(-c2nc(C(F)(F)F)c[nH]2)cc1] | ¹H NMR (400 MHz, Methanol-d₄) δ 7.86 (d, J = 8.4 Hz, 2H), 7.59 (d, J = 1.2 Hz, 1H), 7.46 (d, J = 8.4 Hz, 2H), 7.28 (s, 1H), 5.94 (s, 1H), 5.06 (s, 2H), 4.81-4.72 (m, 1H), 4.41 (t, J = 7.2 Hz, 2H), 3.27 (t, J = 1.6 Hz, 1H), 3.10 (t, J = 7.2 Hz, 2H), 1.94 (s, 3H), 1.31 (d, J = 6.8 Hz, 6H). | m/z: 484.2 [M + H]⁺ |

### Example 71. 2-(4-cyclopropyl-6-methoxypyrimidin-5-yl)-4-(4-(1-ethyl-4-(trifluoromethyl)-1H-imidazol-2-yl)benzyl)-6,7-dihydropyrazolo[1,5-α]pyrimidin-5(4H)-one

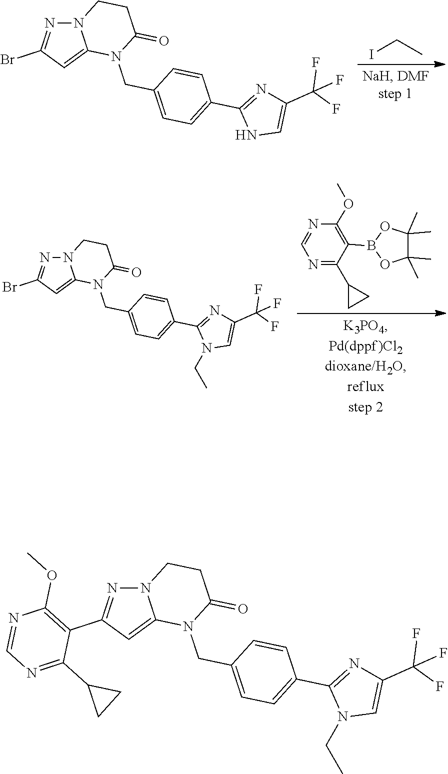

### Step 1: 2-bromo-4-(4-(1-ethyl-4-(trifluoromethyl)-1H-imidazol-2-yl)benzyl)-6,7-dihydropyrazolo[1,5-α]pyrimidin-5(4H)-one

To a solution of 2-bromo-4-(4-(4-(trifluoromethyl)-1H-imidazol-2-yl)benzyl)-6,7-dihydropyrazolo[1,5-α]pyrimidin-5(4H)-one (600 mg, 1.36 mmol) in dry DMF (5 mL) was added NaH (60% in mineral oil, 60 mg, 1.5 mmol) at 0° C. The resulting solution was stirred at 0° C. for 30 min before iodoethane (319 mg, 2.01 mmol) was added. The resulting mixture was then heated at 45° C. for 3 h then cooled to room temperature. The reaction mixture was quenched with H₂O (40 mL) and extracted with EtOAc (3×40 mL). The combined organic layers were dried over anhydrous Na₂SO₄, concentrated under vacuum and purified by silica gel chromatography (eluting with MeOH/DCM 1/50) to afford 2-bromo-4-(4-(1-ethyl-4-(trifluoromethyl)-1H-imidazol-2-yl)benzyl)-6,7-dihydropyrazolo[1,5-α]pyrimidin-5(4H)-one (540 mg). MS (ESI) m/z 468.3, 470.3 [M+H]⁺.

### Step 2: 2-(4-cyclopropyl-6-methoxypyrimidin-5-yl)-4-(4-(1-ethyl-4-(trifluoromethyl)-1H-imidazol-2-yl)benzyl)-6,7-dihydropyrazolo[1,5-α]pyrimidin-5(4H)-one

Example 71 2-(4-cyclopropyl-6-methoxypyrimidin-5-yl)-4-(4-(1-ethyl-4-(trifluoromethyl)-1H-imidazol-2-yl)benzyl)-6,7-dihydropyrazolo[1,5-α]pyrimidin-5(4H)-one was synthesized from 2-bromo-4-(4-(1-ethyl-4-(trifluoromethyl)-1H-imidazol-2-yl)benzyl)-6,7-dihydropyrazolo[1,5-α]pyrimidin-5(4H)-one according to step 3 of Example 1.

Table 9. The compounds listed in Table 9 were synthesized according to Example 71 using the appropriate commercially available reagents and/or intermediates described above.

TABLE 9

| Example | | | |
|---|---|---|---|
| number | Structure | ¹H NMR (δ ppm) | MS (ESI) |
| 71 | 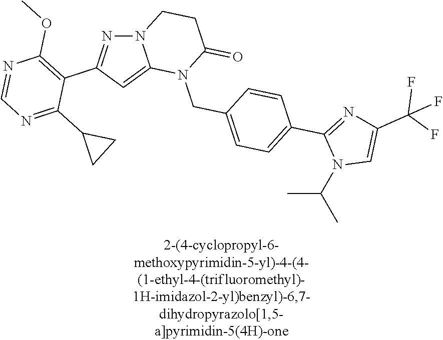 | ¹H NMR (400 MHz, Methanol-d₄) δ 8.58 (s, 1H), 7.84 (s, 1H), 7.62-7.54 (m, 4H), 6.04 (s, 1H), 5.13 (s, 2H), 4.45 (t, J = 7.2 Hz, 2H), 4.12 (t, J = 7.2 Hz, 2H), 3.93 (s, 3H), 3.15 (t, J = 7.2 Hz, 2H), 2.27-2.19 (m, 1H), 1.37 (t, J = 7.2 Hz, 3H), 1.13-1.10 (m, 2H), 1.01-0.97 (m, 2H). | m/z: 538.3 [M + H]⁺ |
| 72 | 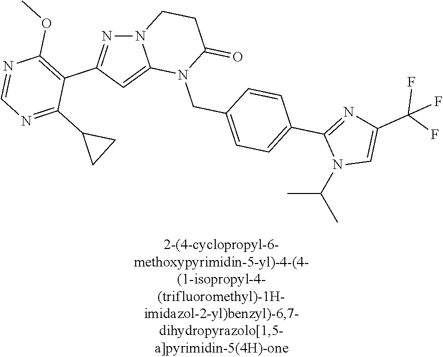 [SMILES: COc1ncnc(C2CC2)c1-c1cc2n(n1)CCC(=O)N2Cc1ccc(-c2nc(C(F)(F)F)cn2C(C)C)cc1] | ¹H NMR (400 MHz, DMSO- d6) δ 8.55 (s, 1H), 8.17 (s, 1H), 7.53 (d, J = 3.6 Hz, 4H), 6.09 (s, 1H), 5.03 (s, 2H), 4.47-4.38 (m, 3H), 3.82 (s, 3H), 3.08 (q, J = 6.8 Hz, 2H), 2.27 (s, 1H), 1.40 (d, J = 6.4 Hz, 6H), 1.00 (s, 2H), 0.88 (s, 2H). | m/z: 552.3 [M + H]⁺ |
| 73 | 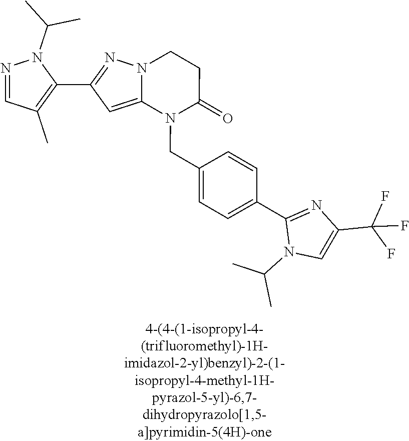 [SMILES: Cc1cnn(C(C)C)c1-c1cc2n(n1)CCC(=O)N2Cc1ccc(-c2nc(C(F)(F)F)cn2C(C)C)cc1] | ¹H NMR (400 MHz, DMSO-d₄) δ 7.90 (s, 1H), 7.57-7.53 (m, 4H), 7.34 (s, 1H), 6.00 (s, 1H), 5.14 (s, 2H), 4.85-4.45 (m, 4H), 3.15 (t, J = 6.8 Hz, 2H), 1.99 (s, 3H), 1.44 (d, J = 6.4 Hz, 6H), 1.38 (d, J = 6.4 Hz, 6H). | m/z: 526.2 [M + H]⁺ |

### Example 74. 2-(2-methoxy-6-methylphenyl)-4-(4-(1-methyl-4-(trifluoromethyl)-1H-imidazol-2-yl)benzyl)-4,5,6,7-tetrahydropyrazolo[1,5-α]pyrimidine

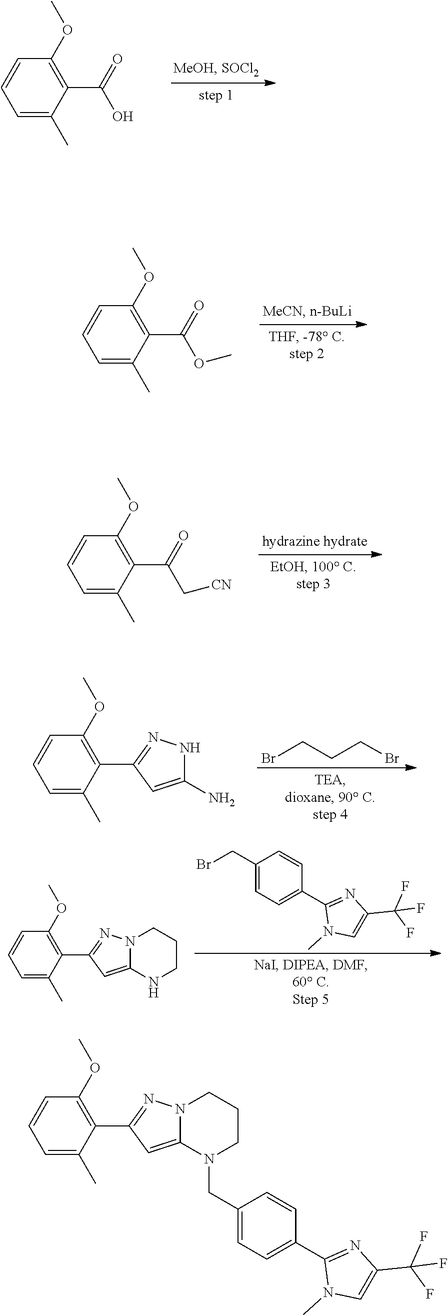

### Step 1. methyl 2-methoxy-6-methylbenzoate

To a solution of 2-methoxy-6-methylbenzoic acid (5.0 g, 30 mmol) in MeOH (50 mL) was added SOC₁₂ (5 mL) dropwise at 0° C. The resulting solution was heated at 65° C. for 6 h then concentrated and purified by silica gel chromatography (eluting with 1/8 EtOAc/PE) to afford methyl 2-methoxy-6-methylbenzoate (4.1 g, 76%) as a white solid. MS (ESI) m/z 181.0 [M+H]⁺.

### Step 2. 3-(2-methoxy-6-methylphenyl)-3-oxopropanenitrile

To a stirred solution of MeCN (1.02g, 25.04 mmol) in dry THF (120 mL) at −78° C. under an atmosphere of N₂ was added n-BuLi (2.5 M in hexane, 13.66 mL, 34.14 mmol) dropwise. The reaction was stirred for 15 min then a solution of methyl 2-methoxy-6-methylbenzoate (4.1 g, 22.76 mmol) in dry THF (50 mL) was added dropwise over 30 min. The resulting solution was stirred at −78° C. for 1 h then warmed to room temperature, quenched with ice cold saturated NH₄Cl solution (200 mL) and extracted with EtOAc (3×100 mL). The combined organic phases were washed with brine, dried over Na₂SO₄, concentrated and purified by silica gel chromatography (eluting with 1/6 EtOAc/PE) to afford of 3-(2-methoxy-6-methylphenyl)-3-oxopropanenitrile (1.4 g). ¹HNMR (400 MHz, Chloroform-d) δ 7.34 (t, J=8.0 Hz, 1H), 6.87 (d, J=7.6 Hz, 1H), 6.82 (d, J=8.4 Hz, 1H), 3.89 (s, 2H), 3.88 (s, 3H), 2.89 (s, 3H).

### Step 3. 3-(2-methoxy-6-methylphenyl)-1H-pyrazol-5-amine

To a solution of 3-(2-methoxy-6-methylphenyl)-1H-pyrazol-5-amine (1.4 g, 7.4 mmol) in EtOH (6 mL) was added 80% hydrazine hydrate (2.82 mL) and the resulting solution heated at 100° C. for 24 h. The reaction was concentrated under reduced pressure and the residue obtained purified on a Biotage Isolera One (C₁₈ column, eluting with 10% to 95% MeCN/H₂O) to afford 3-(2-methoxy-6-methylphenyl)-1H-pyrazol-5-amine (610 mg). MS (ESI) m/z 204.7, [M+H]⁺.

### Step 4. 2-(2-methoxy-6-methylphenyl)-4,5,6,7-tetrahydropyrazolo[1,5-α]pyrimidine

To a solution of 3-(2-methoxy-6-methylphenyl)-1H-pyrazol-5-amine (650 mg, 3.20 mmol) in dioxane (5 mL) was added 1,3-dibromopropane (646.8 mg, 32.0 mmol) and TEA (970.2 mg, 9.59 mmol). The resulting resolution was heated at 90° C. for 16 h then concentrated in vacuo and purified by silica gel chromatography (eluting with 1/6 EtOAc/PE) to afford 2-(2-methoxy-6-methylphenyl)-4,5,6,7-tetrahydropyrazolo[1,5-α]pyrimidine (466.8 mg). ¹H NMR (400 MHz, Chloroform-d) δ 7.21 (t, J=7.9 Hz 1H), 6.77 (t, J=7.4 Hz, 1H), 6.67 (d, J=7.5 Hz, 1H), 5.44 (s, 1H), 4.34(t, J=6.4 Hz 2H), 3.76(s, 3H) 3.37-3.30 (m, 2H), 2.31-2.08 (m, 5H).

### Step 5. 2-(2-methoxy-6-methylphenyl)-4-(4-(1-methyl-4-(trifluoromethyl)-1H-imidazol-2-yl)benzyl)-4,5,6,7-tetrahydropyrazolo[1,5-α]pyrimidine

Example 74 2-(2-methoxy-6-methylphenyl)-4-(4-(1-methyl-4-(trifluoromethyl)-1H-imidazol-2-yl)benzyl)-4,5,6,7-tetrahydropyrazolo[1,5-α]pyrimidine was prepared from 2-(2-methoxy-6-methylphenyl)-4,5,6,7-tetrahydropyrazolo-[1,5-α]pyrimidine according to step 5 of Example 24. ¹H NMR (400 MHz, Methanol-d₄) δ 7.73 (s, 1H), 7.71 (d, J=8.0 Hz, 2H), 7.57 (d, J=8.0 Hz, 2H), 7.38 (t, J=8.0 Hz, 1H), 6.97-6.94 (m, 2H), 4.96 (s, 1H), 4.71 (s, 2H), 4.25 (t, J=6.0 Hz, 2H), 3.80 (s, 3H), 3.79 (s, 3H), 3.49 (t, J=5.6 Hz, 2H), 2.39-2.32 (m, 2H), 2.26 (s, 3H). MS (ESI) m/z 482.1 [M+H]⁺.

### Example 75. 2-(2-methoxy-6-methylphenyl)-4-(4-(1-methyl-4-(trifluoromethyl)-1H-imidazol-2-yl)benzyl)-6,7-dihydropyrazolo[1,5-α]pyrimidin-5(4H)-one

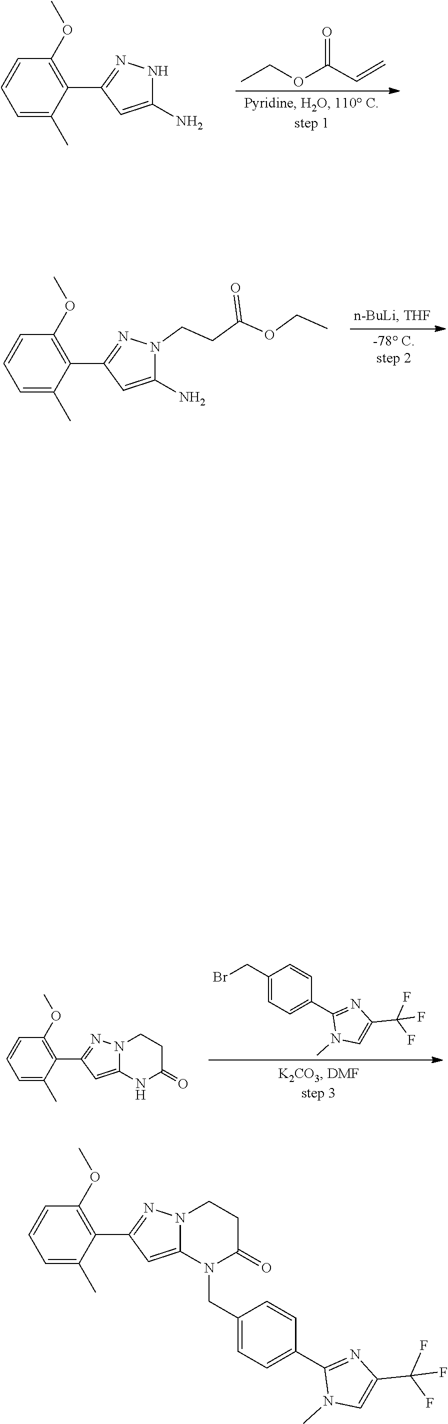

### Step 1. ethyl 3-(5-amino-3-(2-methoxy-6-methylphenyl)-1H-pyrazol-1-yl)propanoate

A mixture of 3-(2-methoxy-6-methylphenyl)-1H-pyrazol-5-amine (200 mg, 0.98 mmol), pyridine (4 mL), H₂O (1 mL) and ethyl acrylate (493.2mg, 4.93 mol) was heated at 110° C. for 24 h. The reaction mixture was concentrated to dryness and purified on a Biotage Isolera One (C₁₈ column, eluting with 10% to 95% MeCN/H₂O) to afford to afford ethyl 3-(5-amino-3-(2-methoxy-6-methylphenyl)-1H-pyrazol-1-yl)propanoate (160 mg). MS (ESI) m/z 303.4, [M+H]⁺.

### Step 2. 2-(2-methoxy-6-methylphenyl)-6,7-dihydropyrazolo[1,5-α]pyrimidin-5(4H)-one

To a solution of ethyl 3-(5-amino-3-(2-methoxy-6-methylphenyl)-1H-pyrazol yl)propanoate (160 mg, 0.53 mmol) in dry THF (10 mL) at −78° C. was added n-BuLi (2.5 M in hexane, 0.23 mL, 0.58 mmol). The resulting solution was stirred at −78° C. for 30 min then warmed to room temperature, quenched with ice cold saturated NH₄Cl solution (10 mL) and extracted with EtOAc (3×10 mL). The combined organic phases were washed with brine, dried over Na₂SO₄, concentrated and purified by silica gel chromatography (eluting with 1/100 MeOH/DCM) to afford 2-(2-methoxy-6-methylphenyl)-6,7-dihydropyrazolo[1,5-α]pyrimidin-5(4H)-one (108 mg). ¹H NMR (400 MHz, DMSO) 6 10.67 (s, 1H), 7.23 (d, J=8.0 Hz, 1H), 6.88 (m, 2H), 5.53 (s, 1H), 4.27 (t, J=6.8 Hz, 2H), 3.63 (s, 3H), 2.83 (t, J=7.2 Hz, 2H), 2.15 (s, 3H).

### Step 3. 2-(2-methoxy-6-methylphenyl)-4-(4-(1-methyl-4-(trifluoromethyl)-1H-imidazol-2-yl)benzyl)-6,7-dihydro-pyrazolo[1,5-α]pyrimidin-5(4H)-one

Example 75 2-(2-methoxy-6-methylphenyl)-4-(4-(1-methyl-4-(trifluoromethyl)-1H-imidazol-2-yl)benzyl)-6,7-dihydropyrazolo[1,5-α]pyrimidin-5(4H)-one was synthesized from 2-(2-methoxy-6-methylphenyl)-6,7-dihydro-pyrazolo[1,5-α]pyrimidin-5(4H)-one following step 2 of Example 1. ¹H NMR (400 MHz, Chloroform-d) δ 7.60 (d, J=8.0 Hz, 2H), 7.44 (d, J=8.0 Hz, 2H), 7.30 (s, 1H), 7.21 (t, J=8.0 Hz, 1H), 6.85 (d, J=7.6 Hz, 1H), 6.78 (d, J=8.0 Hz, 1H), 5.73 (s, 1H), 5.02 (s, 2H), 4.42 (t, J=7.2 Hz, 2H), 3.74 (s, 3H), 3.72 (s, 3H), 3.10 (t, J=7.2 Hz, 2H), 2.16 (s, 3H). MS (ESI) m/z 496.0, [M+H]⁺.

### Example 76. 2-(4-cyclopropyl-6-methoxypyrimidin-5-yl)-6,6-dimethyl-4-(4-(1-methyl-4-(trifluoromethyl)-1H-imidazol-2-yl)benzyl)-6,7-dihydropyrazolo[1,5-α]pyrimidin-5(4H)-one

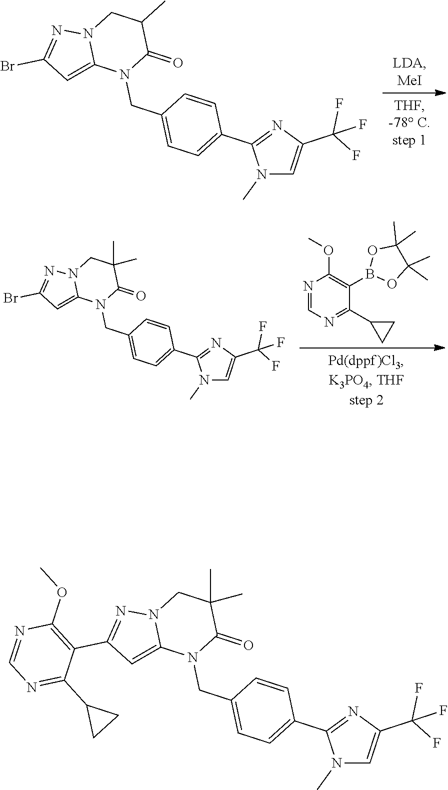

### Step 1. 2-bromo-6,6-dimethyl-4-(4-(1-methyl-4-(trifluoromethyl)-1H-imidazol-2-yl)benzyl)-6,7-dihydropyrazolo-[1,5-α]pyrimidin-5(4H)-one

solution of 2-bromo-6-methyl-4-(4-(1-methyl-4-(trifluoromethyl)-1H-imidazol-2-yl)benzyl)-6,7-dihydropyrazolo[1,5-α]pyrimidin-5(4H)-one (223 mg, 0.48 mmol, prepared in step 1 of Example 31) in dry THF (8 mL) was cooled to −78° C., LDA (2.0 M in THF/hexane, 0.12 mL, 0.24 mmol) was added dropwise and the solution was stirred 30 min before a solution of MeI (33.9 mg, 0.24 mmol) in dry THF (0.5 mL) was added. The resulting solution was stirred at −78° C. for another 30 min then warmed to room temperature. The reaction was quenched with water, diluted with EtOAc, washed with water and brine. The organic phase was dried with Na₂SO₄, concentrated, and purified by silica gel chromatography (eluting with 2:1 PE/EtOAc) to afford 2-bromo-6,6-dimethyl-4-(4-(1-methyl-4-(trifluoromethyl)-1H-imidazol-2-yl)benzyl)-6,7-dihydropyrazolo[1,5-α]pyrimidin-5(4H)-one (53 mg). MS (ESI) m/z 482.1, 484.1 [M+H]⁺.

### Step 2. 2-(4-cyclopropyl-6-methoxypyrimidin-5-yl)-6,6-dimethyl-4-(4-(1-methyl-4-(trifluoromethyl)-1H-imidazol-2-yl)benzyl)-6,7-dihydropyrazolo[1,5-α]pyrimidin-5(4H)-one

Example 76 2-(4-cyclopropyl-6-methoxypyrimidin-5-yl)-6,6-dimethyl-4-(4-(1-methyl-4-(trifluoromethyl)-1H-imidazol-2-yl)benzyl)-6,7-dihydropyrazolo[1,5-α]pyrimidin-5(4H)-one was prepared from 2-bromo-6,6-dimethyl-4-(4-(1-methyl-4-(trifluoromethyl)-1H-imidazol-2-yl)benzyl)-6,7-dihydropyrazolo[1,5-α]pyrimidin-5(4H)-one according to step 3 of Example 48. ¹H NMR (400 MHz, Chloroform-d) δ 8.55 (s, 1H), 7.60 (d, J=8.0 Hz, 2H), 7.41 (d, J=8.0 Hz, 2H), 7.29 (s, 1H), 5.84 (s, 1H), 5.01 (s, 2H), 4.16 (s, 2H), 3.90 (s, 3H), 3.75 (s, 3H), 3.23-2.18 (m, 1H), 1.36 (s, 6H), 1.18-1.16 (m, 2H), 0.93-0.91 (m, 2H). MS (ESI) m/z 552.3 [M+H]⁺.

### Example 77. 2-(4-chloro-1-isopropyl-1H-pyrazol-5-yl)-4-(4-(1-ethyl-4-(trifluoromethyl)-1H-imidazol-2-yl)-3-fluorobenzyl)-6,7-dihydropyrazolo[1,5-α]pyrimidin-5(4H)-one

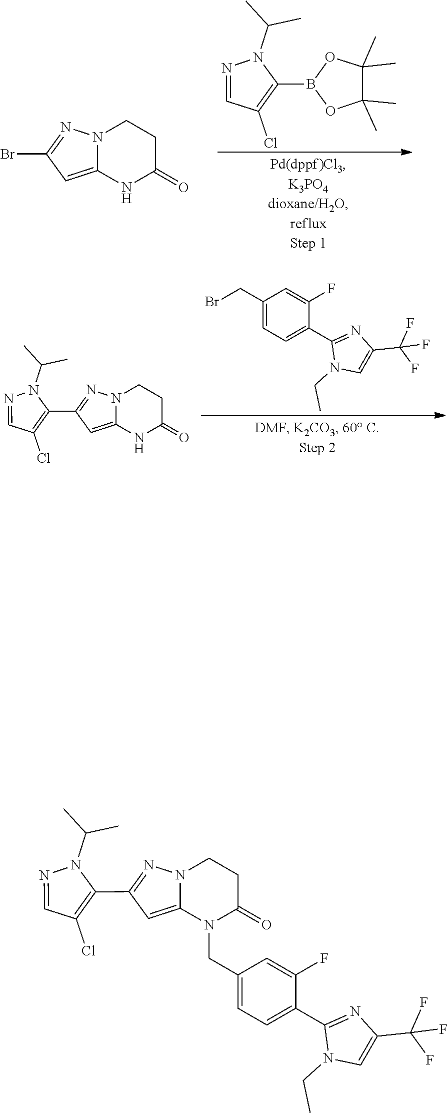 [SMILES: O=C1CCn2nc(Br)cc2N1 ; CC(C)n1ncc(Cl)c1B1OC(C)(C)C(C)(C)O1 ; CC(C)n1ncc(Cl)c1-c1cc2n(n1)CCC(=O)N2 ; CCn1cc(C(F)(F)F)nc1-c1ccc(CBr)cc1F ; CCn1cc(C(F)(F)F)nc1-c1ccc(CN2C(=O)CCn3nc(-c4c(Cl)cnn4C(C)C)cc32)cc1F]

### Step 1. 2-(4-chloro-1-isopropyl-1H-pyrazol-5-yl)-6,7-dihydropyrazolo[1,5-α]pyrimidin-5(4H)-one

2-(4-chloro-1-isopropyl-1H-pyrazol-5-yl)-6,7-dihydropyrazolo[1,5-α]pyrimidin-5(4H)-one was prepared from 2-bromo-6,7-dihydropyrazolo[1,5-α]pyrimidin-5(4H)-one (prepared in step 1 of Example 1) following step 3 of Example 1. MS (ESI) m/z 280.2 [M+H]⁺.

### Step 2. 2-(4-chloro-1-isopropyl-1H-pyrazol-5-yl)-4-(4-(1-ethyl-4-(trifluoromethyl)-1H-imidazol-2-yl)-3-fluorobenzyl)-6,7-dihydropyrazolo[1,5-α]pyrimidin-5(4H)-one

Example 77 2-(4-chloro-1-isopropyl-1H-pyrazol-5-yl)-4-(4-(1-ethyl-4-(trifluoromethyl)-1H-imidazol-2-yl)-3-fluorobenzyl)-6,7-dihydropyrazolo[1,5-α]pyrimidin-5(4H)-one was prepared 2-(4-chloro-1-isopropyl-1H-pyrazol-5-yl)-6,7-dihydropyrazolo[1,5-α]pyrimidin-5(4H)-one following step 2 of Example 1.

Table 10. The compounds listed in Table 10 were synthesized according to Example 77 using the appropriate commercially available reagents and/or intermediates described above. Enantiomers, when generated, were separated by chiral prep-HPLC and absolute stereochemistries were assigned by comparing retention time on chiral HPLC with similar chiral compounds that were obtained via enantioselective synthesis.

TABLE 10

| Example | | | |
|---|---|---|---|
| number | Structure | ¹H NMR (δ ppm) | MS (ESI) |
| 77 | 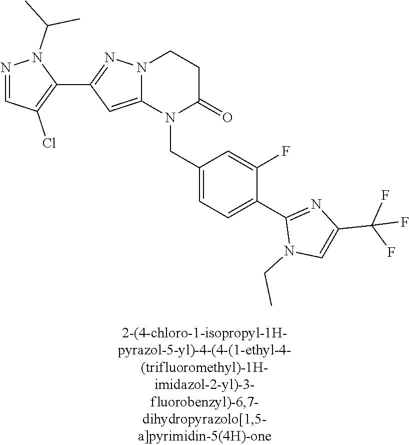 [SMILES: CCn1cc(C(F)(F)F)nc1-c1ccc(CN2C(=O)CCn3nc(-c4c(Cl)cnn4C(C)C)cc32)cc1F] | ¹H NMR (400 MHz, DMSO- d₆) δ 8.09 (d, J = 1.1 Hz, 1H), 7.63 (s, 1H), 7.54 (t, J = 7.7 Hz, 1H), 7.43 (d, J = 10.9 Hz, 1H), 7.36 (d, J = 7.9 Hz, 1H), 6.28 (s, 1H), 5.17-4.94 (m, 3H), 4.47 (t, J = 7.1 Hz, 2H), 3.86 (q, J = 7.2 Hz, 2H), 3.12 (t, J = 7.1 Hz, 2H), 1.35 (d, J = 6.6 Hz, 6H), 1.25 (t, J = 7.3 Hz, 3H). | m/z: 550.2 [M + H]⁺ |
| 78 | 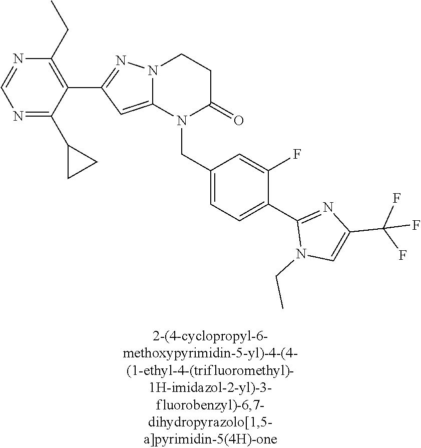 [SMILES: CCc1ncnc(C2CC2)c1-c1cc2n(n1)CCC(=O)N2Cc1ccc(-c2nc(C(F)(F)F)cn2CC)c(F)c1] | ¹H NMR (400 MHz, Chloroform-d) δ 8.57 (s, 1H), 7.55 (t, J = 7.6 Hz, 1H), 7.44- 7.38 (m, 1H), 7.24 (s, 1H), 7.16 (d, J = 10.4 Hz, 1H), 5.85 (s, 1H), 5.03 (s, 2H), 4.46 (t, J = 7.1 Hz, 2H), 3.92 (d, J = 9.0 Hz, 5H), 3.12 (t, J = 7.1 Hz, 2H), 2.21 (dt, J = 8.1, 3.5 Hz, 1H), 1.39 (t, J = 7.3 Hz, 3H), 1.19 (dq, J = 6.3, 3.8 Hz, 2H), 0.94 (dq, J = 6.8, 3.7 Hz, 2H). | m/z: 556.2 [M + H]⁺ |
| 79 | 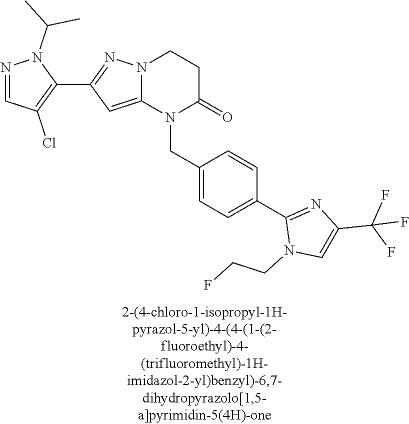 [SMILES: CC(C)n1ncc(Cl)c1-c1cc2n(n1)CCC(=O)N2Cc1ccc(-c2nc(C(F)(F)F)cn2CCF)cc1] | ¹H NMR (400 MHz, Methanol-d₄) δ 7.81 (s, 1H), 7.59 (d, J = 8.0 Hz, 2H), 7.54 (d, J = 8.1 Hz, 2H), 7.50 (s, 1H), 6.22 (s, 1H), 5.17-5.08 (m, 3H), 4.72 (t, J = 4.7 Hz, 1H), 4.62-4.58 (m, 2H), 4.48 (t, J = 7.1 Hz, 2H), 4.39 (t, J = 4.7 Hz, 1H), 4.32 (t, J = 4.7 Hz, 1H), 3.15 (t, J = 7.1 Hz, 2H), 1.40 (d, J = 6.6 Hz, 6H). | m/z: 550.2 [M + H]⁺ |
| 80 | 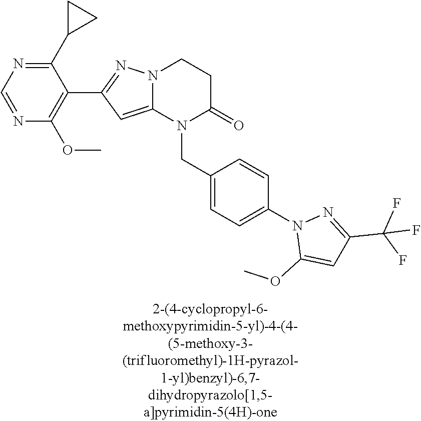 [SMILES: COc1ncnc(C2CC2)c1-c1cc2n(n1)CCC(=O)N2Cc1ccc(-n2nc(C(F)(F)F)cc2OC)cc1] | ¹H NMR (400 MHz, Methanol-d₄) δ 8.56 (s, 1H), 7.64 (d, J = 8.3 Hz, 2H), 7.48 (d, J = 8.3 Hz, 2H), 6.20 (s, 1H), 6.01 (s, 1H), 5.08 (s, 2H), 4.43 (t, J = 7.1 Hz, 2H), 4.00 (s, 3H), 3.92 (s, 3H), 3.13 (t, J = 7.1 Hz, 2H), 2.19 (tt, J = 8.6, 4.8 Hz, 1H), 1.12- 1.08 (m, 2H), 0.96 (dq, J = 7.2, 3.9 Hz, 2H). | m/z: 540.2 [M + H]⁺ |
| 81 | 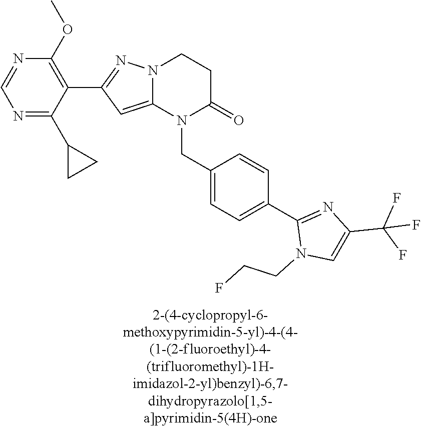 [SMILES: COc1ncnc(C2CC2)c1-c1cc2n(n1)CCC(=O)N2Cc1ccc(-c2nc(C(F)(F)F)cn2CCF)cc1] | ¹H NMR (400 MHz, Chloroform-d) δ 8.55 (s, 1H), 7.53 (d, J = 7.9 Hz, 2H), 7.49- 7.39 (m, 3H), 5.86 (s, 1H), 5.04 (s, 2H), 4.72 (t, J = 4.7 Hz, 1H), 4.60 (t, J = 4.7 Hz, 1H), 4.45 (t, J = 7.1 Hz, 2H), 4.32 (t, J = 4.7 Hz, 1H), 4.27 (q, J = 5.0 Hz, 1H), 3.91 (s, 3H), 3.11 (t, J = 7.1 Hz, 2H), 2.19 (tt, J = 8.5, 4.8 Hz, 1H), 1.19-1.15 (m, 3.9 Hz, 2H), 0.98-0.89 (m, 2H). | m/z: 556.2 [M + H]⁺ |
| 82 | 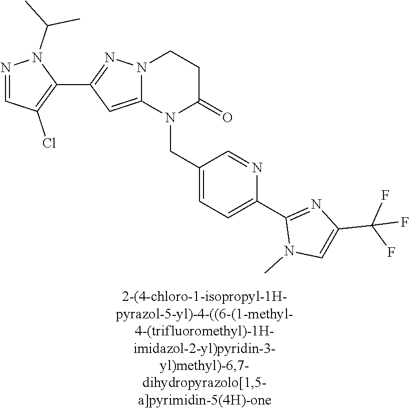 [SMILES: CC(C)n1ncc(Cl)c1-c1cc2n(n1)CCC(=O)N2Cc1ccc(-c2nc(C(F)(F)F)cn2C)nc1] | ¹H NMR (400 MHz, Chloroform-d) δ 8.61 (d, J = 1.8 Hz, 1H), 8.20 (d, J = 8.2 Hz, 1H), 7.77 (dd, J = 8.3, 2.3 Hz, 1H), 7.49 (s, 1H), 7.29 (s, 1H), 6.21 (s, 1H), 5.22-5.15 (m, 1H), 5.03 (s, 2H), 4.43 (t, J = 7.1 Hz, 2H), 4.13 (s, 3H), 3.10 (t, J = 7.1 Hz, 2H), 1.46 (s, 3H), 1.44 (s, 3H). | m/z: 519.2 [M + H]⁺ |
| 83 | 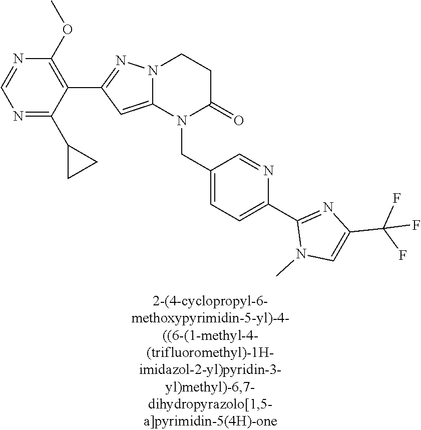 [SMILES: COc1ncnc(C2CC2)c1-c1cc2n(n1)CCC(=O)N2Cc1ccc(-c2nc(C(F)(F)F)cn2C)nc1] | ¹H NMR (400 MHz, Chloroform-d) δ 8.76 (s, 1H), 8.63 (s, 1H), 8.16 (d, J = 8.2 Hz, 1H), 7.79 (d, J = 7.9 Hz, 1H), 7.31 (s, 1H), 5.93 (s, 1H), 5.04 (s, 2H), 4.47 (t, J = 7.0 Hz, 2H), 4.12 (s, 3H), 4.00 (s, 3H), 3.13 (t, J = 7.0 Hz, 2H), 2.32-2.21 (m, 1H), 1.31 (d, J = 3.7 Hz, 2H), 1.07 (d, J = 6.0 Hz, 2H). | m/z: 525.3 [M + H]⁺ |
| 84 | 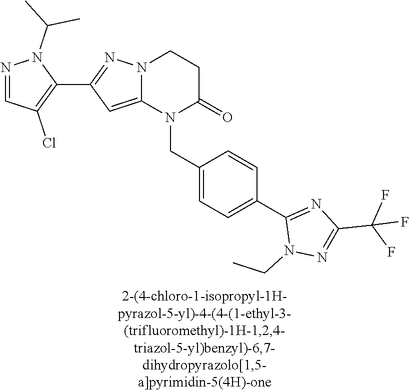 [SMILES: CCn1nc(C(F)(F)F)nc1-c1ccc(CN2C(=O)CCn3nc(-c4c(Cl)cnn4C(C)C)cc32)cc1] | ¹H NMR (400 MHz, Chloroform-d) δ 7.66-7.59 (m, 2H), 7.53-7.46 (m, 3H), 6.15 (s, 1H), 5.22-5.16 (m, 1H), 5.07 (s, 2H), 4.44 (t, J = 7.1 Hz, 2H), 4.29 (q, J = 7.2 Hz, 2H), 3.11 (t, J = 7.1 Hz, 2H), 1.53 (t, J = 7.2 Hz, 3H), 1.44 (d, J = 6.6 Hz, 6H). | m/z: 533.2 [M + H]⁺ |
| 85 | 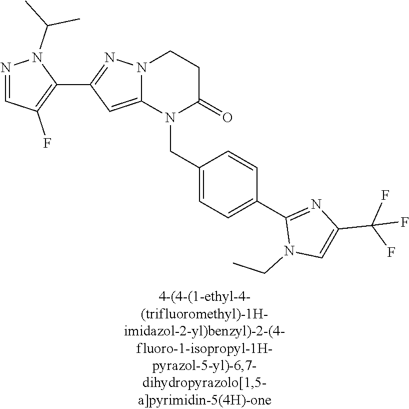 [SMILES: CCn1cc(C(F)(F)F)nc1-c1ccc(CN2C(=O)CCn3nc(-c4c(F)cnn4C(C)C)cc32)cc1] | ¹H NMR (400 MHz, Chloroform-d) δ 7.56 (d, J = 8.0 Hz, 2H), 7.42 (d, J = 8.0 Hz, 2H), 7.38-7.34 (m, 2H), 5.99 (d, J = 1.9 Hz, 1H), 5.30 (dt, J = 13.1, 6.5 Hz, 1H), 5.04 (s, 2H), 4.42 (t, J = 7.0 Hz, 2H), 4.05 (q, J = 7.3 Hz, 2H), 3.10 (t, J = 7.0 Hz, 2H), 1.45 (d, J = 6.6 Hz, 6H), 1.42 (d, J = 7.3 Hz, 3H). | m/z: 516.3 [M + H]⁺ |
| 86 | 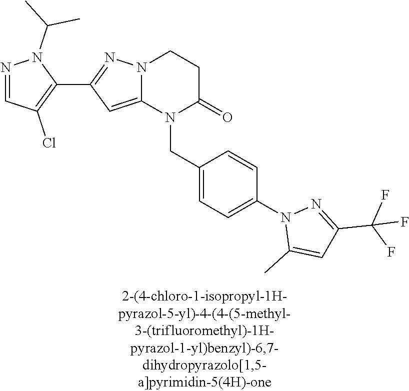 [SMILES: Cc1cc(C(F)(F)F)nn1-c1ccc(CN2C(=O)CCn3nc(-c4c(Cl)cnn4C(C)C)cc32)cc1] | ¹H NMR (400 MHz, Chloroform-d) δ 7.45 (dd, J = 21.1, 8.6 Hz, 5H), 6.44 (s, 1H), 6.17 (s, 1H), 5.18 (dt, J = 13.1, 6.6 Hz, 1H), 5.05 (s, 2H), 4.43 (t, J = 7.0 Hz, 2H), 3.10 (t, J = 7.0 Hz, 2H), 2.33 (s, 3H), 1.45 (d, J = 6.6 Hz, 6H). | m/z: 518.2 [M + H]⁺ |
| 87 | 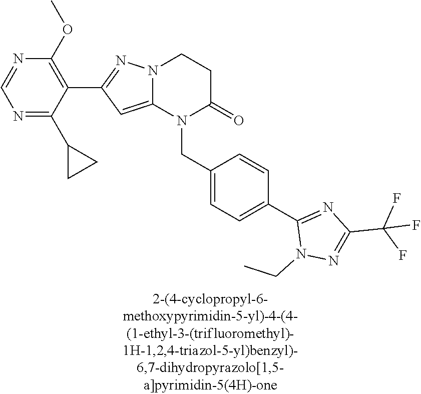 [SMILES: CCn1nc(C(F)(F)F)nc1-c1ccc(CN2C(=O)CCn3nc(-c4c(OC)ncnc4C4CC4)cc32)cc1] | ¹H NMR (400 MHz, Chloroform-d) δ 8.55 (s, 1H), 7.62 (d, J = 8.2 Hz, 2H), 7.50 (d, J = 8.2 Hz, 2H), 5.86 (s, 1H), 5.06 (s, 2H), 4.46 (t, J = 7.1 Hz, 2H), 4.29 (q, J = 7.2 Hz, 2H), 3.91 (s, 3H), 3.12 (t, J = 7.1 Hz, 2H), 2.22-2.16 (m, 1H), 1.53 (t, J = 7.2 Hz, 3H), 1.16 (dt, J = 6.4, 3.2 Hz, 2H), 0.93-0.87 (m, 2H). | m/z: 539.2 [M + H]⁺ |
| 88 | 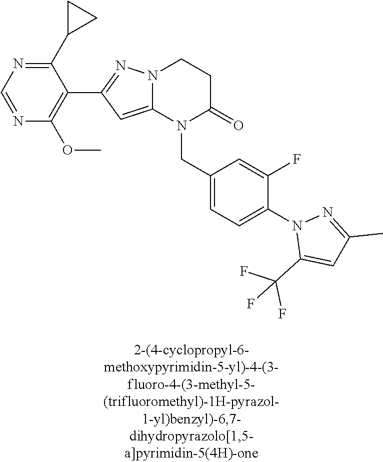 [SMILES: COc1ncnc(C2CC2)c1-c1cc2n(n1)CCC(=O)N2Cc1ccc(-n2nc(C)cc2C(F)(F)F)c(F)c1] | ¹H NMR (400 MHz, Chloroform-d) δ 8.55 (s, 1H), 7.40 (dd, J = 15.8, 7.5 Hz, 1H), 7.25-7.19 (m, 2H), 6.60 (s, 1H), 5.83 (s, 1H), 5.03 (d, J = 3.8 Hz, 2H), 4.46 (t, J = 7.1 Hz, 2H), 3.91 (s, 3H), 3.12 (t, J = 7.1 Hz, 2H), 2.34 (s, 3H), 2.21-2.15 (m, 1H), 1.16 (dt, J = 6.4, 3.2 Hz, 2H), 0.97- 0.88 (m, 2H). | m/z: 542.2 [M + H]⁺ |
| 89 | 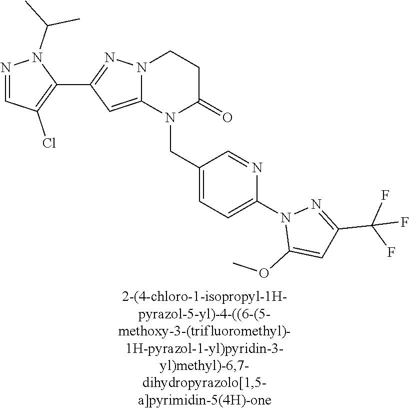 [SMILES: COc1cc(C(F)(F)F)nn1-c1ccc(CN2C(=O)CCn3nc(-c4c(Cl)cnn4C(C)C)cc32)cn1] | ¹H NMR (400 MHz, Chloroform-d) δ 8.65-8.52 (m, 1H), 7.86 (dd, J = 8.5, 2.0 Hz, 1H), 7.71 (d, J = 8.4 Hz, 1H), 7.49 (s, 1H), 6.21 (s, 1H), 5.95 (s, 1H), 5.20-5.14 (m, 1H), 5.03 (s, 2H), 4.41 (t, J = 7.0 Hz, 2H), 4.01 (s, 3H), 3.07 (t, J = 7.0 Hz, 2H), 1.44 (d, J = 6.5 Hz, 6H). | m/z: 535.2 [M + H]⁺ |
| 90 | 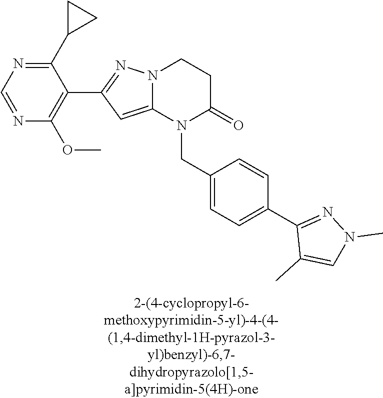 [SMILES: COc1ncnc(C2CC2)c1-c1cc2n(n1)CCC(=O)N2Cc1ccc(-c2nn(C)cc2C)cc1] | ¹H NMR (400 MHz, Chloroform-d) δ 8.54 (s, 1H), 7.61 (d, J = 8.3 Hz, 2H), 7.36 (d, J = 8.2 Hz, 2H), 7.18 (s, 1H), 5.87 (s, 1H), 5.01 (s, 2H), 4.42 (t, J = 7.1 Hz, 2H), 3.88 (d, J = 15.3 Hz, 6H), 3.09 (t, J = 7.1 Hz, 2H), 2.19- 2.13 (m, 4H), 1.13 (dt, J = 6.4, 3.2 Hz, 2H), 0.91-0.86 (m, 2H). | m/z: 470.4 [M + H]⁺ |
| 91 | 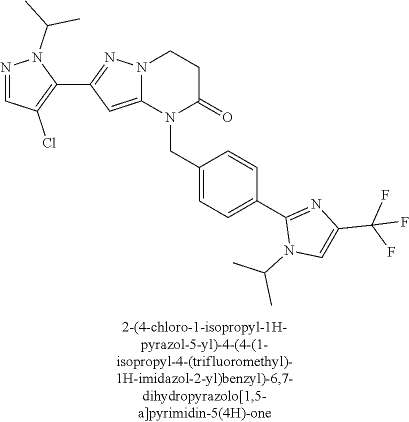 [SMILES: CC(C)n1cc(C(F)(F)F)nc1-c1ccc(CN2C(=O)CCn3nc(-c4c(Cl)cnn4C(C)C)cc32)cc1] | ¹H NMR (400 MHz, Chloroform-d) δ 7.53-7.47 (m, 3H), 7.46-7.38 (m, 3H), 6.15 (s, 1H), 5.21-5.14 (m, 1H), 5.05 (s, 2H), 4.53-4.48 (m, 1H), 4.43 (t, J = 7.1 Hz, 2H), 3.10 (t, J = 7.1 Hz, 2H), 1.45 (d, J = 4.7 Hz, 6H), 1.43 (d, J = 4.8 Hz, 6H). | m/z: 546.2 [M + H]⁺ |
| 92 | 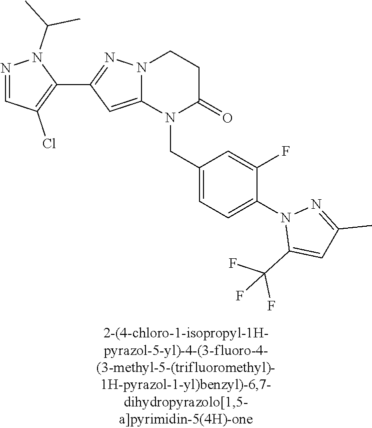 [SMILES: Cc1cc(C(F)(F)F)n(-c2ccc(CN3C(=O)CCn4nc(-c5c(Cl)cnn5C(C)C)cc43)cc2F)n1] | ¹H NMR (400 MHz, Chloroform-d) δ 7.48 (s, 1H), 7.40 (dd, J = 15.8, 7.6 Hz, 1H), 7.24-7.18 (m, 2H), 6.59 (s, 1H), 6.13 (s, 1H), 5.19- 5.13 (m, 1H), 5.03 (s, 2H), 4.44 (t, J = 7.1 Hz, 2H), 3.10 (t, J = 7.1 Hz, 2H), 2.33 (s, 3H), 1.45 (d, J = 6.6 Hz, 6H). | m/z: 536.2 [M + H]⁺ |
| 93 | 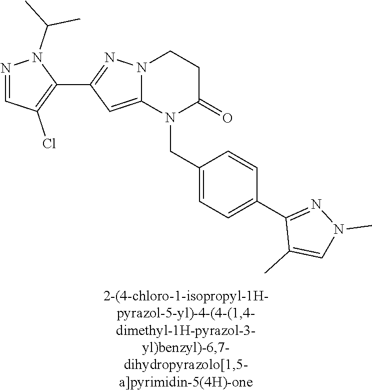 [SMILES: Cc1cn(C)nc1-c1ccc(CN2C(=O)CCn3nc(-c4c(Cl)cnn4C(C)C)cc32)cc1] | ¹H NMR (400 MHz, Chloroform-d) δ 7.63 (d, J = 8.3 Hz, 2H), 7.48 (s, 1H), 7.37 (d, J = 8.2 Hz, 2H), 7.18 (s, 1H), 6.17 (s, 1H), 5.17-5.11 (m, 1H), 5.02 (s, 2H), 4.41 (t, J = 7.1 Hz, 2H), 3.87 (s, 3H), 3.09 (t, J = 7.1 Hz, 2H), 2.19 (s, 3H), 1.43 (d, J = 6.6 Hz, 6H). | m/z: 464.2 [M + H]⁺ |
| 94 | 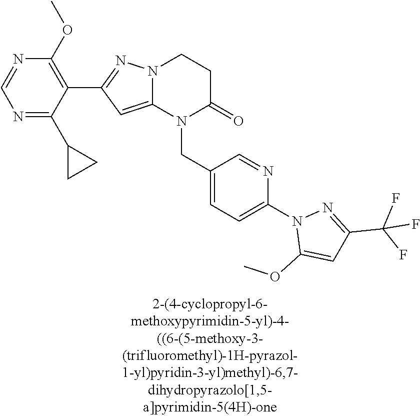 [SMILES: COc1ncnc(C2CC2)c1-c1cc2n(n1)CCC(=O)N2Cc1ccc(-n2nc(C(F)(F)F)cc2OC)nc1] | ¹H NMR (400 MHz, Chloroform-d) δ 8.60-8.52 (m, 2H), 7.86 (dd, J = 8.4, 2.3 Hz, 1H), 7.70 (d, J = 8.4 Hz, 1H), 5.96 (s, 1H), 5.89 (s, 1H), 5.02 (s, 2H), 4.43 (t, J = 7.1 Hz, 2H), 4.01 (s, 3H), 3.91 (s, 3H), 3.08 (t, J = 7.1 Hz, 2H), 2.16 (td, J = 8.1, 4.1 Hz, 1H), 1.16 (dt, J = 6.4, 3.2 Hz, 2H), 0.92 (tt, J = 6.9, 3.8 Hz, 2H). | m/z: 541.3 [M + H]⁺ |
| 95 | 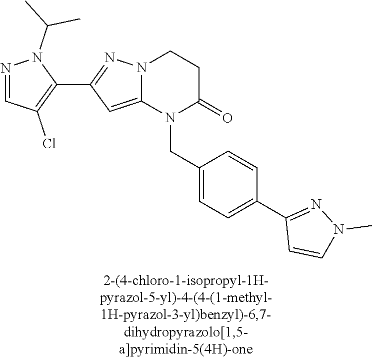 [SMILES: CC(C)n1ncc(Cl)c1-c1cc2n(n1)CCC(=O)N2Cc1ccc(-c2ccn(C)n2)cc1] | ¹H NMR (400 MHz, Chloroform-d) δ 7.78-7.71 (m, 2H), 7.47 (s, 1H), 7.36 (dd, J = 5.3, 3.0 Hz, 3H), 6.50 (d, J = 2.3 Hz, 1H), 6.17 (s, 1H), 5.16-5.10 (m, 1H), 5.00 (s, 2H), 4.41 (t, J = 7.1 Hz, 2H), 3.93 (s, 3H), 3.09 (t, J = 7.1 Hz, 2H), 1.43 (d, J = 6.6 Hz, 6H). | m/z: 450.2 [M + H]⁺ |
| 96 | 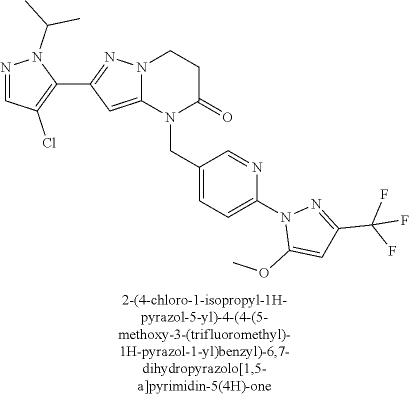 [SMILES: COc1cc(C(F)(F)F)nn1-c1ccc(CN2C(=O)CCn3nc(-c4c(Cl)cnn4C(C)C)cc32)cn1] | ¹H NMR (400 MHz, Chloroform-d) δ 7.69-7.63 (m, 2H), 7.48 (s, 1H), 7.44- 7.40 (m, 2H), 6.15 (s, 1H), 5.93 (s, 1H), 5.19-5.13 (m, 1H), 5.02 (s, 2H), 4.42 (t, J = 7.1 Hz, 2H), 3.97 (s, 3H), 3.09 (t, J = 7.1 Hz, 2H), 1.44 (d, J = 6.6 Hz, 6H). | m/z: 534.2 [M + H]⁺ |
| 97 | 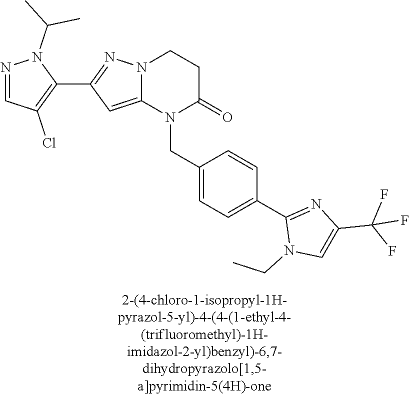 [SMILES: CCn1cc(C(F)(F)F)nc1-c1ccc(CN2C(=O)CCn3nc(-c4c(Cl)cnn4C(C)C)cc32)cc1] | ¹H NMR (400 MHz, Chloroform-d) δ 7.55 (d, J = 8.2 Hz, 2H), 7.47 (s, 1H), 7.43 (d, J = 8.2 Hz, 2H), 7.37-7.33 (m, 1H), 6.14 (s, 1H), 5.2-5.14 (m, 1H), 5.04 (s, 2H), 4.43 (t, J = 7.1 Hz, 2H), 4.03 (q, J = 7.3 Hz, 2H), 3.10 (t, J = 7.1 Hz, 2H), 1.46-1.38 (m, 9H). | m/z: 532.2 [M + H]⁺ |
| 98 | 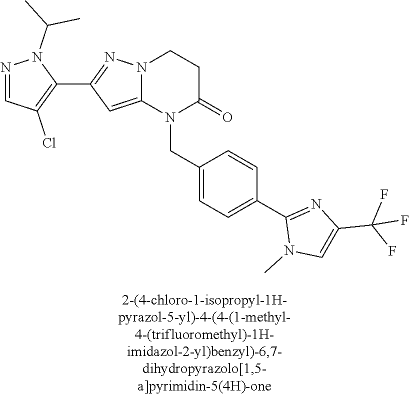 [SMILES: CC(C)n1ncc(Cl)c1-c1cc2n(n1)CCC(=O)N2Cc1ccc(-c2nc(C(F)(F)F)cn2C)cc1] | ¹H NMR (400 MHz, Chloroform-d) δ 7.57 (d, J = 8.0 Hz, 2H), 7.47-7.37 (m, 3H), 7.22 (s, 1H), 6.11 (s, 1H), 5.17-5.11 (m, 1H), 5.01 (s, 2H), 4.39 (t, J = 7.1 Hz, 2H), 3.71 (s, 3H), 3.07 (t, J = 7.1 Hz, 2H), 1.41 (d, J = 6.6 Hz, 6H). | m/z: 518.2 [M + H]⁺ |
| 99 | 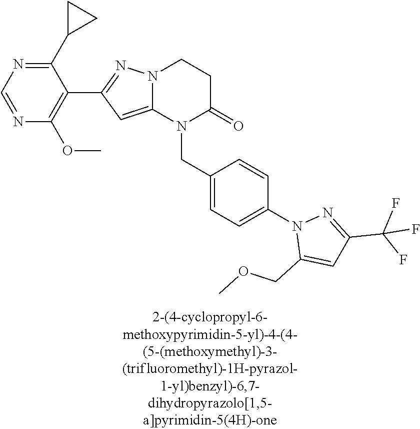 [SMILES: COCc1cc(C(F)(F)F)nn1-c1ccc(CN2C(=O)CCn3nc(-c4c(OC)ncnc4C4CC4)cc32)cc1] | ¹H NMR (400 MHz, Chloroform-d) δ 8.54 (s, 1H), 7.60-7.55 (m, 2H), 7.48-7.44 (m, 2H), 6.70 (s, 1H), 5.85 (s, 1H), 5.04 (s, 2H), 4.44 (t, J = 7.1 Hz, 2H), 4.37 (s, 2H), 3.90 (s, 3H), 3.37 (s, 3H), 3.10 (t, J = 7.1 Hz, 2H), 2.21-2.15 (m, 1H), 1.17-1.12 (m, 2H), 0.93- 0.88 (m, 2H). | m/z: 554.3 [M + H]⁺ |
| 100 | 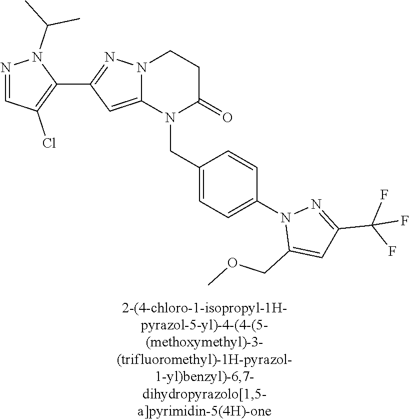 [SMILES: COCc1cc(C(F)(F)F)nn1-c1ccc(CN2C(=O)CCn3nc(-c4c(Cl)cnn4C(C)C)cc32)cc1] | ¹H NMR (400 MHz, Chloroform-d) δ 7.61-7.56 (m, 2H), 7.47 (d, J = 9.1 Hz, 3H), 6.71 (s, 1H), 6.16 (s, 1H), 5.21-5.15 (m, 1H), 5.05 (s, 2H), 4.43 (t, J = 7.1 Hz, 2H), 4.37 (s, 2H), 3.37 (s, 3H), 3.10 (t, J = 7.1 Hz, 2H), 1.44 (d, J = 6.6 Hz, 6H). | m/z: 548.2 [M + H]⁺ |
| 101 | 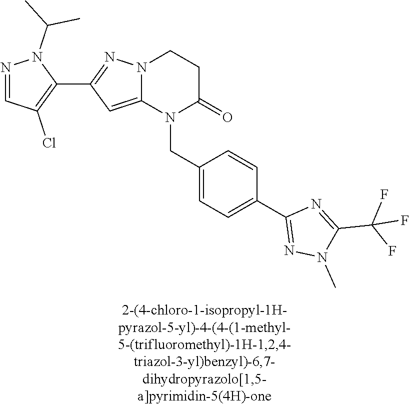 [SMILES: CC(C)n1ncc(Cl)c1-c1cc2n(n1)CCC(=O)N2Cc1ccc(-c2nc(C(F)(F)F)n(C)n2)cc1] | ¹H NMR (400 MHz, Chloroform-d) δ 8.05 (d, J = 8.3 Hz, 2H), 7.47 (s, 1H), 7.40 (d, J = 8.3 Hz, 2H), 6.14 (s, 1H), 5.18-5.11 (m, 1H), 5.03 (s, 2H), 4.43 (t, J = 7.1 Hz, 2H), 4.12-4.03 (m, 3H), 3.10 (t, J = 7.1 Hz, 2H), 1.43 (d, J = 6.6 Hz, 6H). | m/z: 519.2 [M + H]⁺ |
| 102 | 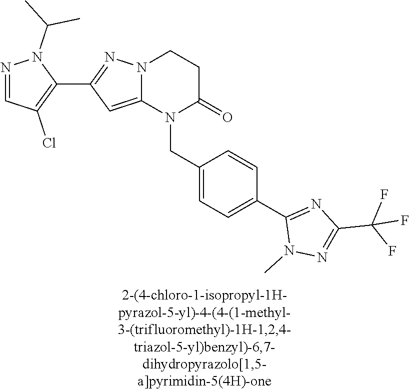 [SMILES: CC(C)n1ncc(Cl)c1-c1cc2n(n1)CCC(=O)N2Cc1ccc(-c2nc(C(F)(F)F)nn2C)cc1] | ¹H NMR (400 MHz, Chloroform-d) δ 7.70-7.65 (m, 2H), 7.50 (d, J = 8.4 Hz, 2H), 7.47 (s, 1H), 6.15 (s, 1H), 5.22-5.16 (m, 1H), 5.07 (s, 2H), 4.44 (t, J = 7.1 Hz, 2H), 4.02 (s, 3H), 3.11 (t, J = 7.1 Hz, 2H), 1.44 (d, J = 6.6 Hz, 6H). | m/z: 519.2 [M + H]⁺ |
| 103 | 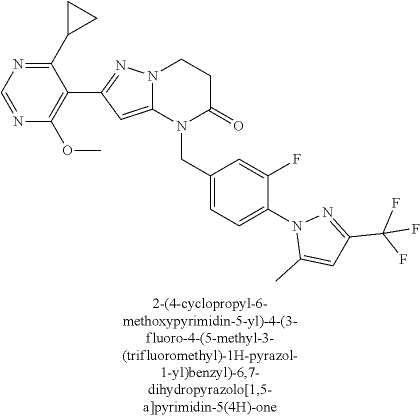 [SMILES: COc1ncnc(C2CC2)c1-c1cc2n(n1)CCC(=O)N2Cc1ccc(-n2nc(C(F)(F)F)cc2C)c(F)c1] | ¹H NMR (400 MHz, Chloroform-d) δ 8.56 (s, 1H), 7.42 (t, J = 7.8 Hz, 1H), 7.30- 7.21 (m, 2H), 6.45 (s, 1H), 5.85 (s, 1H), 5.03 (s, 2H), 4.46 (t, J = 7.1 Hz, 2H), 3.92 (s, 3H), 3.12 (t, J = 7.1 Hz, 2H), 2.26-2.15 (m, 4H), 1.22- 1.14 (m, 2H), 0.94 (dq, J = 6.7, 3.7 Hz, 2H). | m/z: 542.3 [M + H]⁺ |
| 104 | 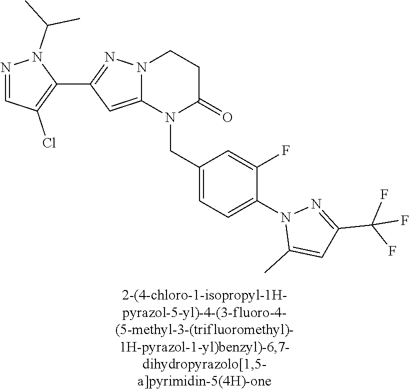 [SMILES: Cc1cc(C(F)(F)F)nn1-c1ccc(CN2C(=O)CCn3nc(-c4c(Cl)cnn4C(C)C)cc32)cc1F] | ¹H NMR (400 MHz, Chloroform-d) δ 7.50 (s, 1H), 7.46 (t, J = 7.8 Hz, 1H), 7.29 (d, J = 1.4 Hz, 1H), 7.23 (d, J = 1.7 Hz, 1H), 6.45 (s, 1H), 6.16 (s, 1H), 5.23-5.16 (m, 1H), 5.04 (s, 2H), 4.45 (t, J = 7.1 Hz, 2H), 3.12 (t, J = 7.1 Hz, 2H), 2.25-2.21 (m, 3H), 1.46 (d, J = 6.6 Hz, 6H). | m/z: 536.3 [M + H]⁺ |
| 105 | 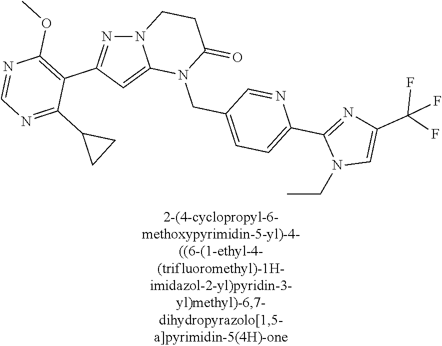 [SMILES: CCn1cc(C(F)(F)F)nc1-c1ccc(CN2C(=O)CCn3nc(-c4c(OC)ncnc4C4CC4)cc32)cn1] | ¹H NMR (400 MHz, Chloroform-d) δ 8.60 (d, J = 1.9 Hz, 1H), 8.54 (s, 1H), 8.18 (d, J = 8.2 Hz, 1H), 7.76 (dd, J = 8.3, 2.3 Hz, 1H), 7.36- 7.31 (m, 1H), 5.90 (s, 1H), 5.01 (s, 2H), 4.62 (q, J = 7.2 Hz, 2H), 4.43 (t, J = 7.1 Hz, 2H), 3.91 (s, 3H), 3.10 (t, J = 7.1 Hz, 2H), 2.22-2.15 (m, 1H), 1.44 (t, J = 7.2 Hz, 3H), 1.15 (dt, J = 6.4, 3.2 Hz, 2H), 0.95-0.86 (m, 2H). | m/z: 539.2 [M + H]⁺ |
| 106 | 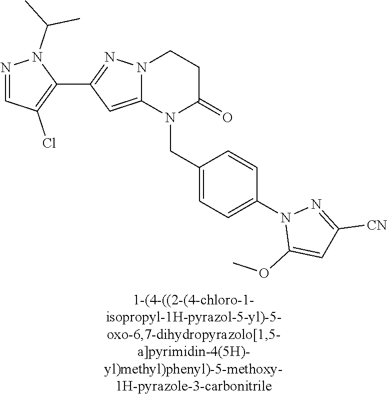 [SMILES: COc1cc(C#N)nn1-c1ccc(CN2C(=O)CCn3nc(-c4c(Cl)cnn4C(C)C)cc32)cc1] | ¹H NMR (400 MHz, Chloroform-d) δ 7.65 (d, J = 8.3 Hz, 2H), 7.48 (s, 1H), 7.44 (d, J = 8.2 Hz, 2H), 6.16 (s, 1H), 6.04 (s, 1H), 5.23-5.13 (m, 1H), 5.03 (s, 2H), 4.43 (t, J = 7.0 Hz, 2H), 3.97 (s, 3H), 3.10 (t, J = 7.0 Hz, 2H), 1.44 (d, J = 6.5 Hz, 6H). | m/z: 491.2 [M + H]⁺ |
| 107 | 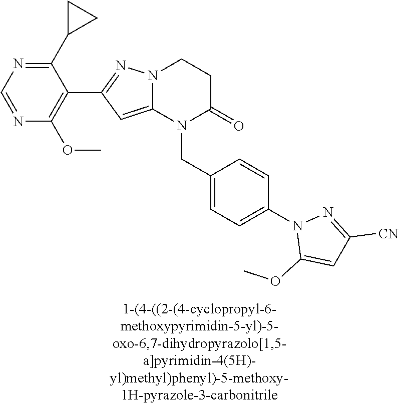 [SMILES: COc1ncnc(C2CC2)c1-c1cc2n(n1)CCC(=O)N2Cc1ccc(-n2nc(C#N)cc2OC)cc1] | ¹H NMR (400 MHz, Chloroform-d) δ 8.55 (s, 1H), 7.64 (d, J = 7.6 Hz, 2H), 7.43 (d, J = 7.7 Hz, 2H), 6.05 (s, 1H), 5.86 (s, 1H), 5.02 (s, 2H), 4.43 (d, J = 6.5 Hz, 2H), 3.94 (d, J = 25.9 Hz, 6H), 3.11 (d, J = 6.3 Hz, 2H), 2.17 (s, 1H), 1.15 (s, 2H), 0.95- 0.80 (m, 2H). | m/z: 497.2 [M + H]⁺ |
| 108 | 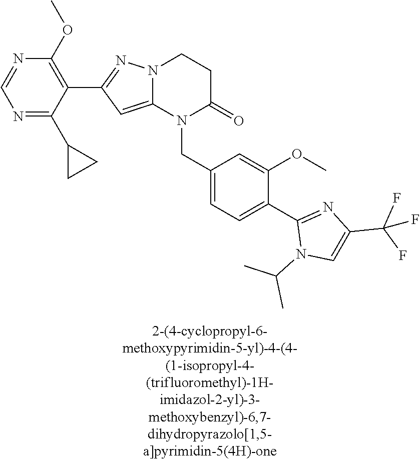 [SMILES: COc1cc(CN2C(=O)CCn3nc(-c4c(OC)ncnc4C4CC4)cc32)ccc1-c1nc(C(F)(F)F)cn1C(C)C] | ¹H NMR (400 MHz, Chloroform-d) δ 8.57 (s, 1H), 7.39-7.36 (m, 2H), 7.00 (dd, J = 7.7, 1.5 Hz, 1H), 6.96 (d, J = 1.5 Hz, 1H), 5.90 (s, 1H), 5.01 (s, 2H), 4.45 (t, J = 7.1 Hz, 2H), 4.19-4.10 (m, 1H), 3.92 (s, 3H), 3.77 (s, 3H), 3.11 (t, J = 7.1 Hz, 2H), 2.18 (tt, J = 8.3, 4.7 Hz, 1H), 1.36 (d, J = 6.7 Hz, 6H), 1.17 (dt, J = 6.4, 3.2 Hz, 2H), 0.96-0.90 (m, 2H). | m/z: 582.4 [M + H]⁺ |
| 109 | 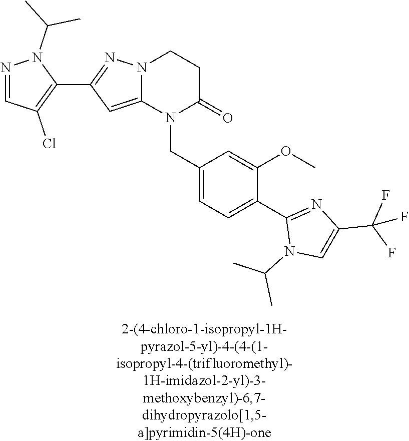 [SMILES: COc1cc(CN2C(=O)CCn3nc(-c4c(Cl)cnn4C(C)C)cc32)ccc1-c1nc(C(F)(F)F)cn1C(C)C] | ¹H NMR (400 MHz, Chloroform-d) δ 7.43 (d, J = 42.8 Hz, 3H), 6.98 (d, J = 20.5 Hz, 2H), 6.20 (s, 1H), 5.26-5.13 (m, 1H), 5.02 (s, 2H), 4.43 (t, J = 7.2 Hz, 2H), 4.14 (s, 1H), 3.77 (s, 3H), 3.10 (t, J = 6.7 Hz, 2H), 1.45 (d, J = 6.6 Hz, 6H), 1.35 (s, 6H). | m/z: 576.2 [M + H]⁺ |
| 110 | 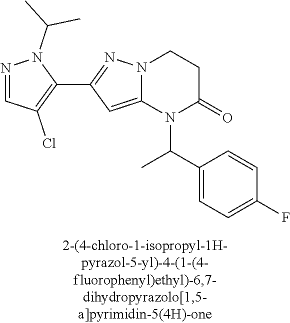 [SMILES: CC(c1ccc(F)cc1)N1C(=O)CCn2nc(-c3c(Cl)cnn3C(C)C)cc21] | ¹H NMR (400 MHz, Chloroform-d) δ 7.45 (s, 1H), 7.32 (d, J = 8.5, 5.2 Hz, 2H), 7.04 (t, J = 8.6 Hz, 2H), 6.23 (q, J = 7.2 Hz, 1H), 5.74 (s, 1H), 5.10-5.04 (m, 1H), 4.45- 4.36 (m, 2H), 3.14-3.01 (m, 2H), 1.77 (d, J = 7.1 Hz, 3H), 1.41 (t, J = 6.8 Hz, 6H). | m/z: 402.2 [M + H]⁺ |
| 111 | 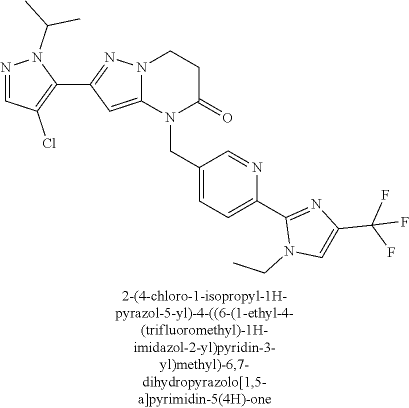 [SMILES: CCn1cc(C(F)(F)F)nc1-c1ccc(CN2C(=O)CCn3nc(-c4c(Cl)cnn4C(C)C)cc32)cn1] | ¹H NMR (400 MHz, Chloroform-d) δ 8.61 (d, J = 2.2 Hz, 1H), 8.20 (d, J = 8.2 Hz, 1H), 7.77 (dd, J = 8.3, 2.3 Hz, 1H), 7.49 (s, 1H), 7.34 (d, J = 1.2 Hz, 1H), 6.21 (s, 1H), 5.22-5.15 (m, 1H), 5.03 (s, 2H), 4.63 (q, J = 7.1 Hz, 2H), 4.43 (t, J = 7.1 Hz, 2H), 3.10 (t, J = 7.1 Hz, 2H), 1.44 (dd, J = 6.9, 2.0 Hz, 9H). | m/z: 533.2 [M + H]⁺ |
| 112 | 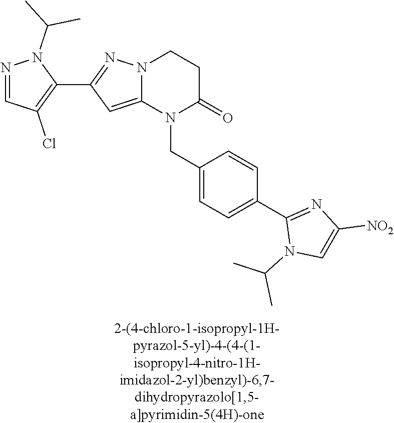 [SMILES: CC(C)n1cc([N+](=O)[O-])nc1-c1ccc(CN2C(=O)CCn3nc(-c4c(Cl)cnn4C(C)C)cc32)cc1] | ¹H NMR (400 MHz, Chloroform-d) δ 7.92 (s, 1H), 7.54 (d, J = 8.1 Hz, 2H), 7.49- 7.43 (m, 3H), 6.14 (s, 1H), 5.22-5.15 (m, 1H), 5.06 (s, 2H), 4.60-4.53 (m, 1H), 4.44 (t, J = 7.1 Hz, 2H), 3.11 (t, J = 7.1 Hz, 2H), 1.48 (d, J = 6.7 Hz, 6H), 1.44 (d, J = 6.6 Hz, 6H). | m/z: 523.2 [M + H]⁺ |
| 113 | 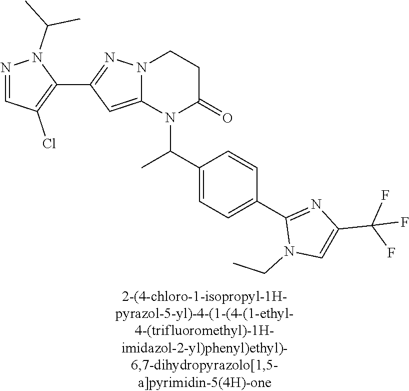 [SMILES: CCn1cc(C(F)(F)F)nc1-c1ccc(C(C)N2C(=O)CCn3nc(-c4c(Cl)cnn4C(C)C)cc32)cc1] | ¹H NMR (400 MHz, Chloroform-d) δ 7.61-7.55 (m, 2H), 7.48-7.41 (m, 3H), 7.36 (d, J = 1.2 Hz, 1H), 6.32 (q, J = 7.1 Hz, 1H), 5.79 (s, 1H), 5.19-5.09 (m, 1H), 4.49- 4.35 (m, 2H), 4.04 (q, J = 7.3 Hz, 2H), 3.19-3.00 (m, 2H), 1.82 (d, J = 7.2 Hz, 3H), 1.42 (t, J = 6.9 Hz, 9H). | m/z: 546.3 [M + H]⁺ |
| 114 | 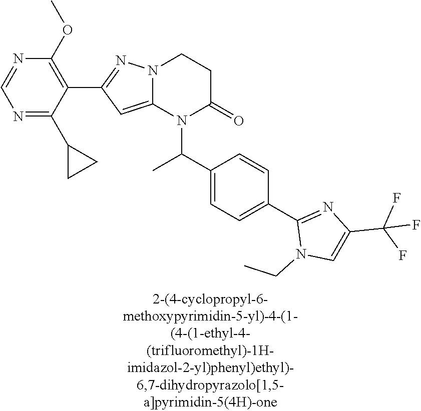 [SMILES: CCn1cc(C(F)(F)F)nc1-c1ccc(C(C)N2C(=O)CCn3nc(-c4c(OC)ncnc4C4CC4)cc32)cc1] | ¹H NMR (400 MHz, Chloroform-d) δ 8.52 (s, 1H), 7.57 (d, J = 8.2 Hz, 2H), 7.45 (d, J = 8.2 Hz, 2H), 7.36 (s, 1H), 6.27 (q, J = 7.1 Hz, 1H), 5.50 (s, 1H), 4.50-4.37 (m, 2H), 4.05 (q, J = 7.3 Hz, 2H), 3.86 (s, 3H), 3.20-3.01 (m, 2H), 2.14-2.07 (m, 1H), 1.82 (d, J = 7.1 Hz, 3H), 1.42 (t, J = 7.3 Hz, 3H), 1.15-1.07 (m, 2H), 0.92-0.81 (m, 2H). | m/z: 552.3 [M + H]⁺ |
| 115 | 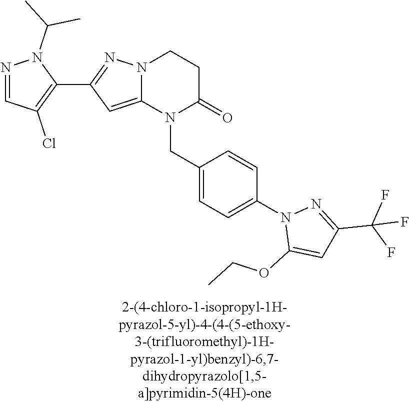 [SMILES: CCOc1cc(C(F)(F)F)nn1-c1ccc(CN2C(=O)CCn3nc(-c4c(Cl)cnn4C(C)C)cc32)cc1] | ¹H NMR (400 MHz, Chloroform-d) δ 7.69 (d, J = 8.0 Hz, 2H), 7.48 (s, 1H), 7.42 (d, J = 8.0 Hz, 2H), 6.16 (s, 1H), 5.90 (s, 1H), 5.23-5.10 (m, 1H), 5.02 (s, 2H), 4.42 (t, J = 6.8 Hz, 2H), 4.20 (q, J = 6.7 Hz, 2H), 3.09 (t, J = 6.8 Hz, 2H), 1.45 (t, J = 7.1 Hz, 9H). | m/z: 548.3 [M + H]⁺ |
| 116 | 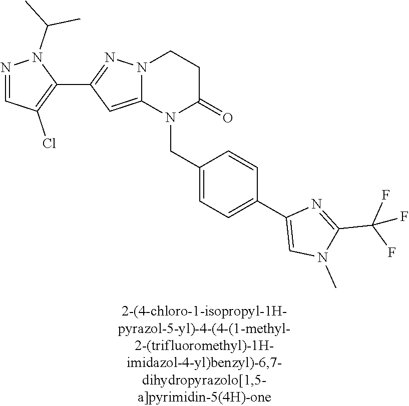 [SMILES: CC(C)n1ncc(Cl)c1-c1cc2n(n1)CCC(=O)N2Cc1ccc(-c2cn(C)c(C(F)(F)F)n2)cc1] | ¹H NMR (400 MHz, Chloroform-d) δ 7.74-7.69 (m, 2H), 7.47 (s, 1H), 7.35 (d, J = 8.1 Hz, 2H), 7.22 (s, 1H), 6.16 (s, 1H), 5.19-5.09 (m, 1H), 4.99 (s, 2H), 4.41 (t, J = 7.1 Hz, 2H), 3.85-3.80 (m, 3H), 3.09 (t, J = 7.1 Hz, 2H), 1.43 (d, J = 6.6 Hz, 6H). | m/z: 518.2 [M + H]⁺ |
| 117 | 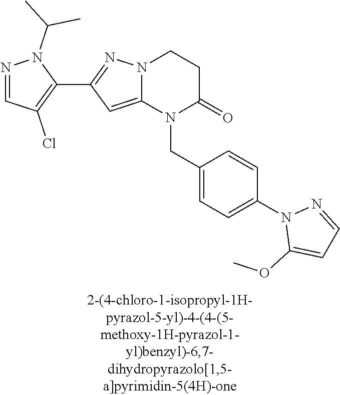 [SMILES: COc1ccnn1-c1ccc(CN2C(=O)CCn3nc(-c4c(Cl)cnn4C(C)C)cc32)cc1] | ¹H NMR (400 MHz, Chloroform-d) δ 7.68 (d, J = 8.4 Hz, 2H), 7.47 (d, J = 2.2 Hz, 2H), 7.39 (d, J = 8.3 Hz, 2H), 6.17 (s, 1H), 5.94 (d, J = 2.0 Hz, 1H), 5.19-5.09 (m, 1H), 5.00 (s, 2H), 4.41 (t, J = 7.1 Hz, 2H), 3.92 (s, 3H), 3.07 (t, J = 7.1 Hz, 2H), 1.43 (d, J = 6.6 Hz, 6H). | m/z: 466.1 [M + H]⁺ |
| 118 | 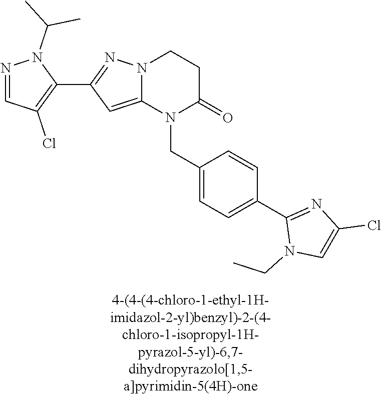 [SMILES: CCn1cc(Cl)nc1-c1ccc(CN2C(=O)CCn3nc(-c4c(Cl)cnn4C(C)C)cc32)cc1] | ¹H NMR (400 MHz, Chloroform-d) δ 7.53 (d, J = 8.2 Hz, 2H), 7.47 (s, 1H), 7.41 (d, J = 8.2 Hz, 2H), 6.91 (s, 1H), 6.14 (s, 1H), 5.17 (m, 1H), 5.03 (s, 2H), 4.43 (t, J = 7.1 Hz, 2H), 3.99 (q, J = 7.3 Hz, 2H), 3.10 (t, J = 7.1 Hz, 2H), 1.44 (d, J = 6.6 Hz, 6H), 1.39 (t, J = 7.3 Hz, 3H). | m/z: 498.1 [M + H]⁺ |
| 119 | 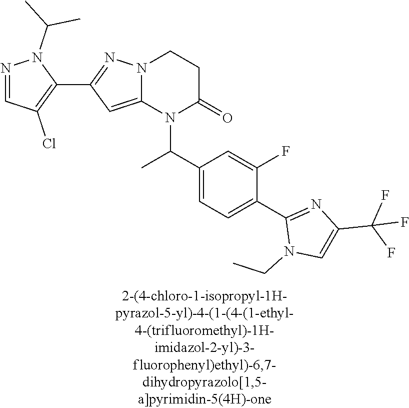 [SMILES: CCn1cc(C(F)(F)F)nc1-c1ccc(C(C)N2C(=O)CCn3nc(-c4c(Cl)cnn4C(C)C)cc32)cc1F] | ¹H NMR (400 MHz, Methanol-d₄) δ 7.84 (s, 1H), 7.58-7.36 (m, 4H), 6.26 (q, J = 7.1 Hz, 1H), 5.87 (s, 1H), 5.16-5.09 (m, 1H), 4.55-4.42 (m, 2H), 3.94 (q, J = 7.3 Hz, 2H), 3.26-3.01 (m, 2H), 1.85 (d, J = 7.1 Hz, 3H), 1.38 (dd, J = 6.7, 2.3 Hz, 6H), 1.30 (d, J = 7.2 Hz, 3H). | m/z: 564.2 [M + H]⁺ |
| 120 | 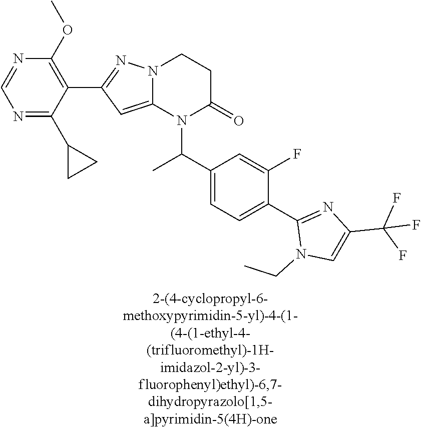 [SMILES: CCn1cc(C(F)(F)F)nc1-c1ccc(C(C)N2C(=O)CCn3nc(-c4c(OC)ncnc4C4CC4)cc32)cc1F] | ¹H NMR (400 MHz, Methanol-d₄) δ 8.47 (s, 1H), 7.84 (s, 1H), 7.53 (t, J = 7.7 Hz, 1H), 7.40 (t, J = 8.9 Hz, 2H), 6.19 (q, J = 7.1 Hz, 1H), 5.68 (s, 1H), 4.48-4.40 (m, 2H), 3.96 (q, J = 7.3 Hz, 2H), 3.86 (s, 3H), 3.23-3.03 (m, 2H), 2.09 (dq, J = 8.4, 4.7, 4.2 Hz, 1H), 1.85 (d, J = 7.1 Hz 3H), 1.32 (t, J = 7.3 Hz, 3H), 1.10-1.02 (m, 2H), 0.94-0.84 (m, 2H). | m/z: 570.2 [M + H]⁺ |
| 121 | 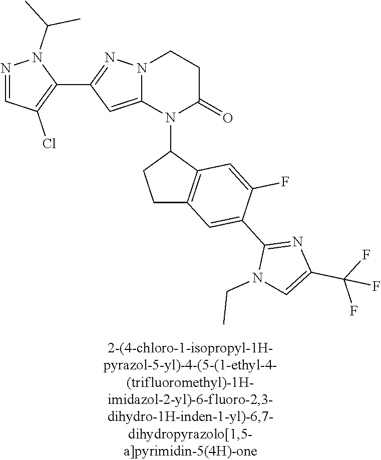 [SMILES: CCn1cc(C(F)(F)F)nc1-c1cc2c(cc1F)C(N1C(=O)CCn3nc(-c4c(Cl)cnn4C(C)C)cc31)CC2] | ¹H NMR (400 MHz, Chloroform-d) δ 7.51 (d, J = 6.4 Hz, 1H), 7.41 (s, 1H), 7.40 (d, J = 1.0 Hz, 1H), 6.89 (d, J = 9.2 Hz, 1H), 6.59 (t, J = 8.2 Hz, 1H), 5.47 (s, 1H), 5.17- 5.10 (m, 1H), 4.52-4.39 (m, 2H), 3.88 (q, J = 7.5 Hz, 2H), 3.21-2.98 (m, 4H), 2.68-2.57 (m, 1H), 2.45-2.34 (m, 1H), 1.42 (dd, J = 6.6, 3.6 Hz, 6H), 1.37 (t, J = 7.3 Hz, 3H). | m/z: 576.3 [M + H]⁺ |
| 122 | 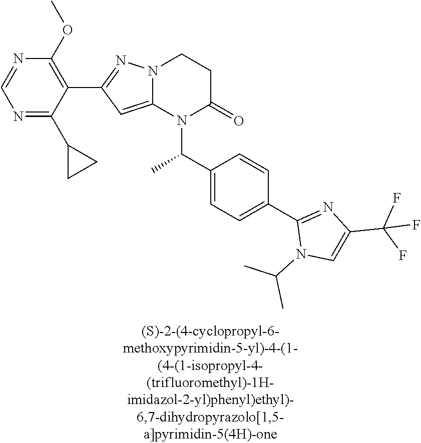 [SMILES: COc1ncnc(C2CC2)c1-c1cc2n(n1)CCC(=O)N2[C@@H](C)c1ccc(-c2nc(C(F)(F)F)cn2C(C)C)cc1] | ¹H NMR (400 MHz, Chloroform-d) δ 8.52 (s, 1H), 7.54 (d, J = 8.3 Hz, 2H), 7.46 (d, J = 8.2 Hz, 2H), 7.41 (s, 1H), 6.26 (q, J = 7.1 Hz, 1H), 5.53 (s, 1H), 4.55 (m, 1H), 4.49-4.37 (m, 2H), 3.86 (s, 3H), 3.19-3.01 (m, 2H), 2.11 (td, J = 8.1, 4.1 Hz, 1H), 1.83 (d, J = 7.2 Hz, 3H), 1.45 (d, J = 6.7 Hz, 6H), 1.17-1.07 (m, 2H), 0.87 (ddd, J = 15.0, 8.4, 4.3 Hz, 2H). | m/z: 566.2 [M + H]⁺ |
| 123 | 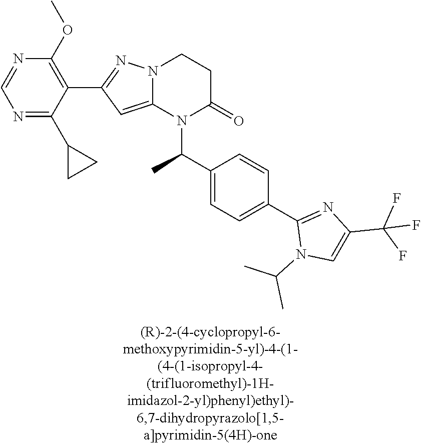 [SMILES: COc1ncnc(C2CC2)c1-c1cc2n(n1)CCC(=O)N2[C@H](C)c1ccc(-c2nc(C(F)(F)F)cn2C(C)C)cc1] | ¹H NMR (400 MHz, Chloroform-d) δ 8.52 (s, 1H), 7.54 (d, J = 8.3 Hz, 2H), 7.46 (d, J = 8.1 Hz, 2H), 7.41 (s, 1H), 6.26 (q, J = 7.1 Hz, 1H), 5.53 (s, 1H), 4.54 (dq, J = 14.0, 7.0 Hz, 1H), 4.49-4.38 (m, 2H), 3.86 (s, 3H), 3.21- 3.01 (m, 2H), 2.11 (tt, J = 8.2, 4.7 Hz, 1H), 1.83 (d, J = 7.2 Hz, 3H), 1.45 (d, J = 6.7 Hz, 6H), 1.12 (dt, J = 5.8, 3.2 Hz, 2H), 0.87 (ddd, J = 14.9, 8.5, 4.1 Hz, 2H). | m/z: 566.2 [M + H]⁺ |
| 124 | 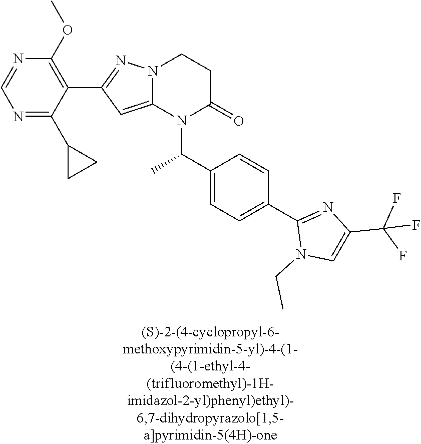 [SMILES: CCn1cc(C(F)(F)F)nc1-c1ccc([C@H](C)N2C(=O)CCn3nc(-c4c(OC)ncnc4C4CC4)cc32)cc1] | ¹H NMR (400 MHz, Chloroform-d) δ 8.51 (s, 1H), 7.60-7.55 (m, 2H), 7.47-7.43 (m, 2H), 7.36 (q, J = 1.2 Hz, 1H), 6.27 (q, J = 7.1 Hz, 1H), 5.50 (s, 1H), 4.50-4.37 (m, 2H), 4.06 (q, J = 7.3 Hz, 2H), 3.86 (s, 3H), 3.19-3.01 (m, 2H), 2.10 (ddd, J = 8.1, 4.7 3.4 Hz, 1H), 1.82 (d, J = 7.1 Hz, 3H), 1.43 (t, J = 7.3 Hz, 3H), 1.12 (d, J = 4.6 Hz, 2H), 0.91-0.80 (m, 2H). | m/z: 552.2 [M + H]⁺ |
| 125 | 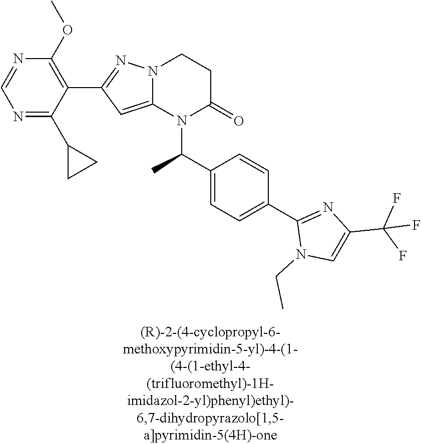 [SMILES: CCn1cc(C(F)(F)F)nc1-c1ccc([C@@H](C)N2C(=O)CCn3nc(-c4c(OC)ncnc4C4CC4)cc32)cc1] | ¹H NMR (400 MHz, Chloroform-d) δ 8.51 (s, 1H), 7.60-7.55 (m, 2H), 7.47-7.43 (m, 2H), 7.36 (q, J = 1.2 Hz, 1H), 6.27 (q, J = 7.1 Hz, 1H), 5.50 (s, 1H), 4.50-4.37 (m, 2H), 4.06 (q, J = 7.3 Hz, 2H), 3.86 (s, 3H), 3.19-3.01 (m, 2H), 2.10 (m, 1H), 1.82 (d, J = 7.1 Hz, 3H), 1.43 (t, J = 7.3 Hz, 3H), 1.12 (d, J = 4.6 Hz, 2H), 0.91-0.80 (m, 2H). | m/z: 552.2 [M + H]⁺ |
| 126 | 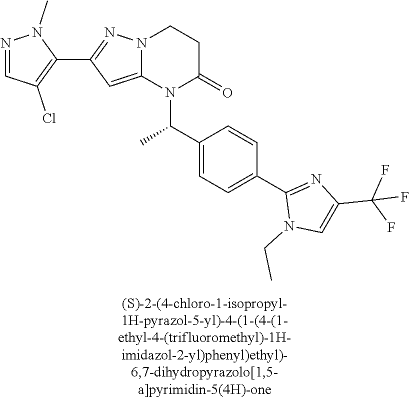 | ¹H NMR (400 MHz, Chloroform-d) δ 7.59-7.55 (m, 2H), 7.47-7.43 (m, 2H), 7.42 (s, 1H), 7.35 (d, J = 1.2 Hz, 1H), 6.31 (q, J = 7.1 Hz, 1H), 5.78 (s, 1H), 5.13 (m, 1H), 4.48-4.37 (m, 2H), 4.03 (q, J = 7.3 Hz, 2H), 3.17-3.02 (m, 2H), 1.81 (d, J = 7.2 Hz, 3H), 1.41 (td, J = 7.1, 1.4 Hz, 9H). | m/z: 546.2 [M + H]⁺ |
| 127 | 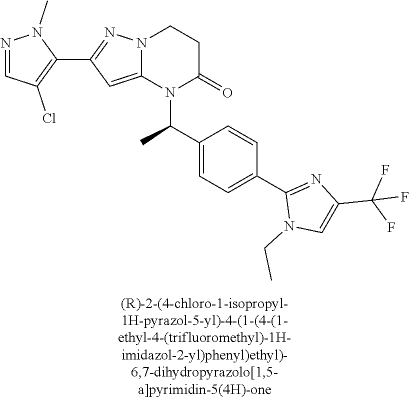 | ¹H NMR (400 MHz, Chloroform-d) δ 7.59-7.55 (m, 2H), 7.47-7.43 (m, 2H), 7.42 (s, 1H), 7.35 (d, J = 1.2 Hz, 1H), 6.31 (q, J = 7.1 Hz, 1H), 5.78 (s, 1H), 5.13 (m, 1H), 4.48-4.37 (m, 2H), 4.03 (q, J = 7.3 Hz, 2H), 3.17-3.02 (m, 2H), 1.81 (d, J = 7.2 Hz, 3H), 1.41 (td, J = 7.1, 1.4 Hz, 9H). | m/z: 546.2 [M + H]⁺ |
| 128 | 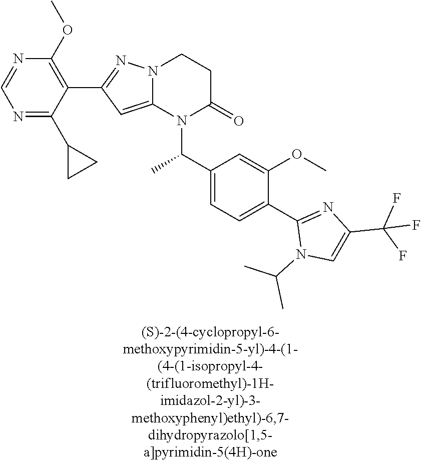 [SMILES: COc1cc([C@H](C)N2C(=O)CCn3nc(-c4c(OC)ncnc4C4CC4)cc32)ccc1-c1nc(C(F)(F)F)cn1C(C)C] | ¹H NMR (400 MHz, Chloroform-d) δ 8.53 (s, 1H), 7.47-7.34 (m, 2H), 7.06 (d, J = 7.6 Hz, 1H), 6.92 (s, 1H), 6.23 (d, J = 6.9 Hz, 1H), 5.62 (s, 1H), 4.44 (q, J = 8.3, 7.2 Hz, 2H), 4.20-4.10 (m, 1H), 3.88 (s, 3H), 3.75 (s, 3H), 3.19-3.02 (m, 2H), 2.14 (s, 1H), 1.82 (d, J = 7.0 Hz, 3H), 1.35 (d, J = 6.2 Hz, 6H), 1.15 (s, 2H), 0.95-0.85 (m, 2H). | m/z: 596.2 [M + H]⁺ |
| 129 | 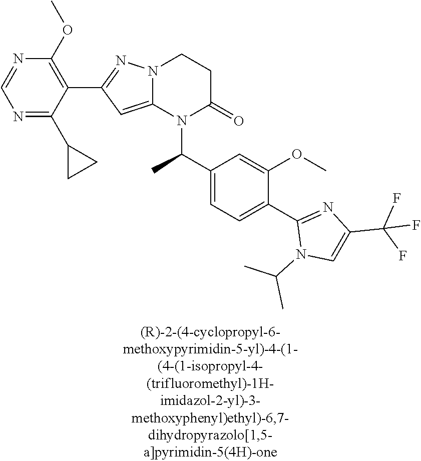 [SMILES: COc1cc([C@@H](C)N2C(=O)CCn3nc(-c4c(OC)ncnc4C4CC4)cc32)ccc1-c1nc(C(F)(F)F)cn1C(C)C] | ¹H NMR (400 MHz, Chloroform-d) δ 8.54 (s, 1H), 7.43-7.57 (m, 2H), 7.06 (d, J = 7.8 Hz, 1H), 6.92 (s, 1H), 6.23 (q, J = 7.5, 6.9 Hz, 1H), 5.62 (s, 1H), 4.44 (q, J = 8.6, 7.4 Hz, 2H), 4.15 (dt, J = 13.4, 6.8 Hz, 1H), 3.88 (s, 3H), 3.76 (s, 3H), 3.19-3.02 (m, 2H), 2.14 (tt, J = 8.0, 4.8 Hz, 1H), 1.82 (d, J = 7.1 Hz, 3H), 1.35 (d, J = 6.3 Hz, 6H), 1.15 (s, 2H), 0.94-0.85 (m, 2H). | m/z: 596.2 [M + H]⁺ |
| 130 | 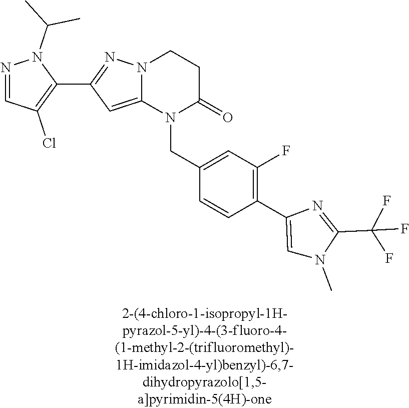 [SMILES: CC(C)n1ncc(Cl)c1-c1cc2n(n1)CCC(=O)N2Cc1ccc(-c2cn(C)c(C(F)(F)F)n2)c(F)c1] | ¹H NMR (400 MHz, Chloroform-d) δ 8.10 (t, J = 8.0 Hz, 1H), 7.48 (s, 1H), 7.41 (d, J = 3.7 Hz, 1H), 7.18 (dd, J = 8.0, 1.7 Hz, 1H), 7.08 (dd, J = 11.7, 1.7 Hz, 1H), 6.14 (s, 1H), 5.15 (m, 1H), 4.99 (s, 2H), 4.43 (t, J = 7.1 Hz, 2H), 3.87-3.78 (m, 3H), 3.10 (t, J = 7.1 Hz, 2H), 1.44 (d, J = 6.6 Hz, 6H). | m/z: 536.2 [M + H]⁺ |
| 131 | 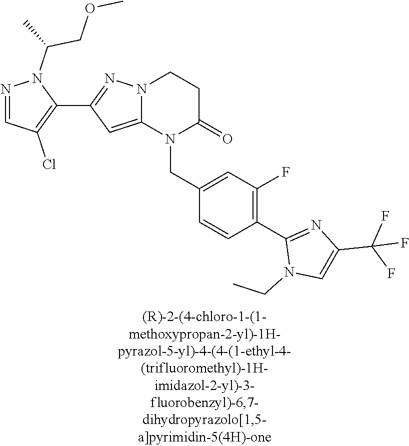 [SMILES: CCn1cc(C(F)(F)F)nc1-c1ccc(CN2C(=O)CCn3nc(-c4c(Cl)cnn4[C@H](C)COC)cc32)cc1F] | ¹H NMR (400 MHz, Chloroform-d) δ 7.55 (t, J = 7.6 Hz, 1H), 7.51 (s, 1H), 7.40 (d, J = 1.3 Hz, 1H), 7.24 (d, J = 1.7 Hz, 1H), 7.15 (dd, J = 10.4, 1.6 Hz, 1H), 6.19 (s, 1H), 5.32-5.21 (m, 1H), 5.03 (s, 2H), 4.44 (t, J = 7.1 Hz, 2H), 3.90 (q, J = 7.3 Hz, 2H), 3.84 (dd, J = 9.8, 7.8 Hz, 1H), 3.58 (dd, J = 9.8, 5.6 Hz, 1H), 3.26 (s, 3H), 3.10 (t, J = 7.1 Hz, 2H), 1.44 (d, J = 6.7 Hz, 3H), 1.38 (t, J = 7.3 Hz, 3H). | m/z: 580.2 [M + H]⁺ |
| 132 | 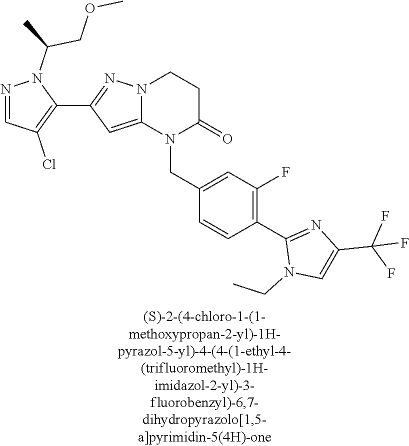 [SMILES: CCn1cc(C(F)(F)F)nc1-c1ccc(CN2C(=O)CCn3nc(-c4c(Cl)cnn4[C@@H](C)COC)cc32)cc1F] | ¹H NMR (400 MHz, Chloroform-d) δ 7.55 (t, J = 7.6 Hz, 1H), 7.51 (s, 1H), 7.40 (d, J = 1.3 Hz, 1H), 7.26-7.22 (m, 1H), 7.15 (dd, J = 10.4, 1.6 Hz, 1H), 6.19 (s, 1H), 5.31-5.21 (m, 1H), 5.03 (s, 2H), 4.44 (t, J = 7.1 Hz, 2H), 3.90 (q, J = 7.3 Hz, 2H), 3.84 (dd, J = 9.8, 7.8 Hz, 1H), 3.58 (dd, J = 9.8, 5.6 Hz, 1H), 3.26 (s, 3H), 3.10 (t, J = 7.1 Hz, 2H), 1.44 (d, J = 6.7 Hz, 3H), 1.38 (t, J = 7.3 Hz, 3H). | m/z: 580.2 [M + H]⁺ |
| 133 | 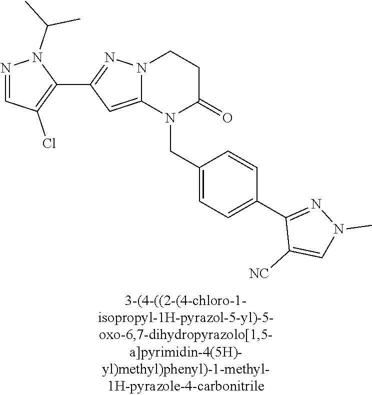 [SMILES: CC(C)n1ncc(Cl)c1-c1cc2n(n1)CCC(=O)N2Cc1ccc(-c2nn(C)cc2C#N)cc1] | ¹H NMR (400 MHz, Chloroform-d) δ 7.92 (d, J = 8.2 Hz, 2H), 7.82 (s, 1H), 7.47 (s, 1H), 7.42 (d, J = 8.2 Hz, 2H), 6.14 (s, 1H), 5.14 (m, 1H), 5.03 (s, 2H), 4.43 (t, J = 7.1 Hz, 2H), 3.96 (s, 3H), 3.10 (t, J = 7.1 Hz, 2H), 1.43 (d, J = 6.6 Hz, 6H). | m/z: 475.1 [M + H]⁺ |
| 134 | 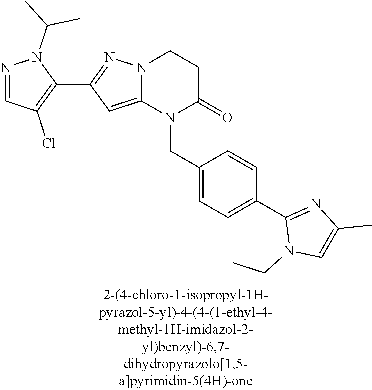 [SMILES: CCn1cc(C)nc1-c1ccc(CN2C(=O)CCn3nc(-c4c(Cl)cnn4C(C)C)cc32)cc1] | ¹H NMR (400 MHz, Chloroform-d) δ 7.54 (d, J = 8.3 Hz, 2H), 7.48 (s, 1H), 7.42 (d, J = 8.2 Hz, 2H), 6.74 (d, J = 1.1 Hz, 1H), 6.16 (s, 1H), 5.17 (m, 1H), 5.03 (s, 2H), 4.44 (t, J = 7.1 Hz, 2H), 3.96 (q, J = 7.3 Hz, 2H), 3.10 (t, J = 7.1 Hz, 2H), 2.27 (d, J = 1.0 Hz, 3H), 1.44 (d, J = 6.6 Hz, 6H), 1.38 (t, J = 7.3 Hz, 3H). | m/z: 478.2 [M + H]⁺ |
| 135 | 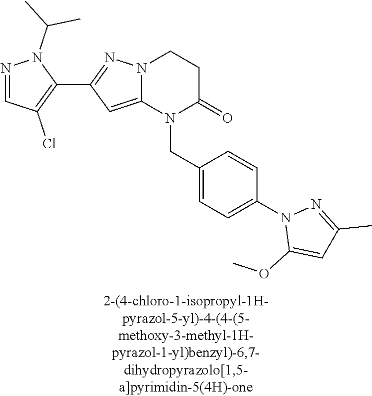 [SMILES: COc1cc(C)nn1-c1ccc(CN2C(=O)CCn3nc(-c4c(Cl)cnn4C(C)C)cc32)cc1] | ¹H NMR (400 MHz, Chloroform-d) δ 7.65 (d, J = 7.9 Hz, 2H), 7.47 (s, 1H), 7.41 (d, J = 7.7 Hz, 2H), 6.15 (s, 1H), 5.57 (s, 1H), 5.15 (m, 1H), 4.99 (s, 2H), 4.41 (t, J = 6.9 Hz, 2H), 3.94 (s, 3H), 3.07 (t, J = 6.9 Hz, 2H), 2.36 (s, 3H), 1.43 (d, J = 6.5 Hz, 6H). | m/z: 480.2 [M + H]⁺ |
| 136 | 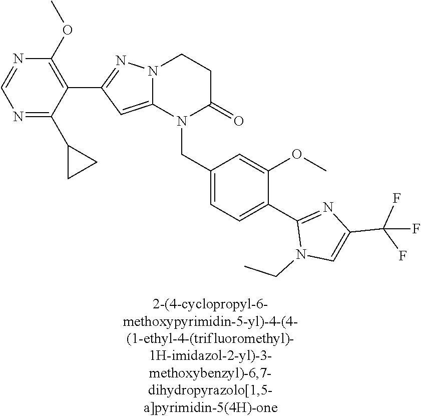 [SMILES: CCn1cc(C(F)(F)F)nc1-c1ccc(CN2C(=O)CCn3nc(-c4c(OC)ncnc4C4CC4)cc32)cc1OC] | ¹H NMR (400 MHz, Chloroform-d) δ 8.56 (s, 1H), 7.43-7.32 (m, 2H), 7.04-6.93 (m, 2H), 5.89 (s, 1H), 5.01 (s, 2H), 4.45 (t, J = 7.1 Hz, 2H), 3.92 (s, 3H), 3.86-3.72 (m, 5H), 3.10 (t, J = 7.1 Hz, 2H), 2.18 (tt, J = 8.2, 4.7 Hz, 1H), 1.33 (t, J = 7.3 Hz, 3H), 1.17 (dt, J = 6.3, 3.2 Hz, 2H), 0.92 (dq, J = 6.8, 3.7 Hz, 2H). | m/z: 568.2 [M + H]⁺ |
| 137 | 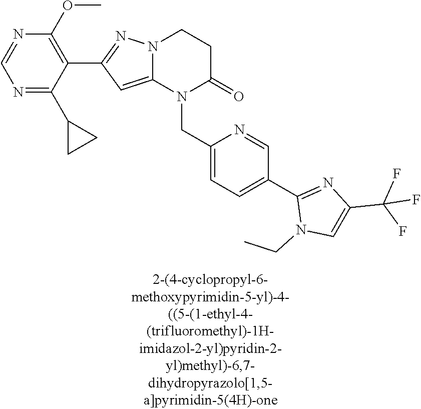 [SMILES: CCn1cc(C(F)(F)F)nc1-c1ccc(CN2C(=O)CCn3nc(-c4c(OC)ncnc4C4CC4)cc32)nc1] | ¹H NMR (400 MHz, Methanol-d₄) δ 8.75 (dd, J = 2.3, 0.9 Hz, 1H), 8.48 (s, 1H), 8.05 (dd, J = 8.2, 2.3 Hz, 1H), 7.86 (q, J = 1.2 Hz, 1H), 7.60 (dd, J = 8.2, 0.8 Hz, 1H), 5.95 (s, 1H), 5.23 (s, 2H), 4.48 (t, J = 7.1 Hz, 2H), 4.13 (q, J = 7.3 Hz, 2H), 3.87 (s, 3H), 3.16 (t, J = 7.1 Hz, 2H), 2.15 (tt, J = 8.1, 4.7 Hz, 1H), 1.40 (t, J = 7.3 Hz, 3H), 1.08 (dt, J = 4.7, 3.1 Hz, 2H), 0.94-0.85 (m, 2H). | m/z: 539.2 [M + H]⁺ |
| 138 | 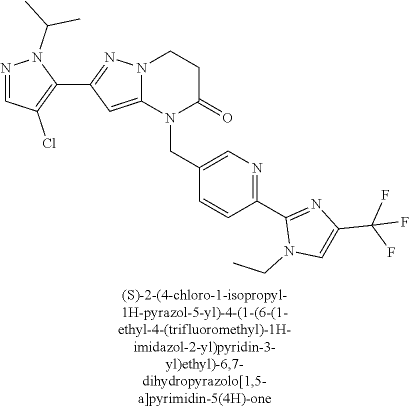 | ¹H NMR (400 MHz, Chloroform-d) δ 8.60 (s, 1H), 8.20 (d, J = 8.3 Hz, 1H), 7.73 (dd, J = 8.3, 2.0 Hz, 1H), 7.42 (s, 1H), 7.34 (s, 1H), 6.24 (q, J = 7.1 Hz, 1H), 5.84 (s, 1H), 5.12 (m, 1H), 6.42 (qd, J = 6.9, 1.6 Hz, 2H), 4.48-4.36 (m, 2H), 3.18-3.00 (m, 2H), 1.85 (d, J = 7.2 Hz, 3H), 1.46- 1.37 (m, 9H). | m/z: 547.2 [M + H]⁺ |
| 139 | 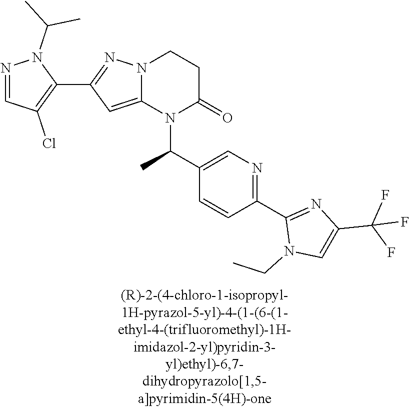 [SMILES: CCn1cc(C(F)(F)F)nc1-c1ccc([C@@H](C)N2C(=O)CCn3nc(-c4c(Cl)cnn4C(C)C)cc32)cn1] | ¹H NMR (400 MHz, Chloroform-d) δ 8.60 (s, 1H), 8.20 (d, J = 8.3 Hz, 1H), 7.73 (dd, J = 8.3, 2.0 Hz, 1H), 7.42 (s, 1H), 7.34 (s, 1H), 6.24 (q, J = 7.1 Hz, 1H), 5.84 (s, 1H), 5.12 (m, 1H), 4.62 (qd, J = 6.9, 1.6 Hz, 2H), 4.48-4.36 (m, 2H), 3.18-3.00 (m, 2H), 1.85 (d, J = 7.2 Hz, 3H), 1.46- 1.37 (m, 9H). | m/z: 547.2 [M + H]⁺ |
| 140 | 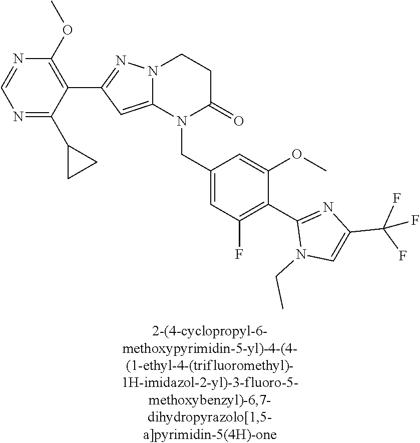 [SMILES: CCn1cc(C(F)(F)F)nc1-c1c(F)cc(CN2C(=O)CCn3nc(-c4c(OC)ncnc4C4CC4)cc32)cc1OC] | ¹H NMR (400 MHz, Chloroform-d) δ 8.56 (s, 1H), 7.39 (s, 1H), 6.75 (d, J = 8.1 Hz, 2H), 5.86 (s, 1H), 5.10- 4.87 (m, 2H), 4.45 (t, J = 7.1 Hz, 2H), 3.92 (s, 3H), 3.77 (d, J = 8.1 Hz, 5H), 3.11 (t, J = 7.1 Hz, 2H), 2.19 (dt, J = 8.0, 3.6 Hz, 1H), 1.33 (t, J = 7.3 Hz, 3H), 1.17 (dd, J = 4.3, 2.8 Hz, 2H), 0.93 (dd, J = 8.0, 2.9 Hz, 2H). | m/z: 586.2 [M + H]⁺ |
| 141 | 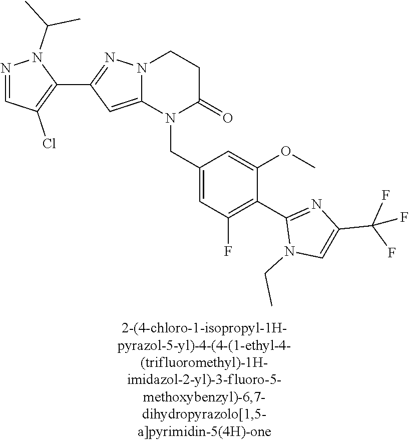 [SMILES: CCn1cc(C(F)(F)F)nc1-c1c(F)cc(CN2C(=O)CCn3nc(-c4c(Cl)cnn4C(C)C)cc32)cc1OC] | ¹H NMR (400 MHz, Chloroform-d) δ 7.49 (s, 1H), 7.39 (s, 1H), 6.75 (d, J = 6.3 Hz, 2H), 6.17 (s, 1H), 5.20 (m, 1H), 4.99 (d, J = 5.7 Hz, 2H), 4.44 (t, J = 7.1 Hz, 2H), 3.76 (d, J = 6.7 Hz, 5H), 3.10 (t, J = 7.1 Hz, 2H), 1.46 (d, J = 6.6 Hz, 6H), 1.32 (t, J = 7.3 Hz, 3H). | m/z: 580.1 [M + H]⁺ |
| 142 | 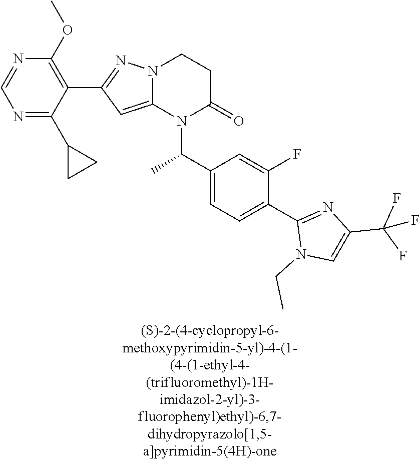 [SMILES: CCn1cc(C(F)(F)F)nc1-c1ccc([C@H](C)N2C(=O)CCn3nc(-c4c(OC)ncnc4C4CC4)cc32)cc1F] | ¹H NMR (400 MHz, Chloroform-d) δ 8.51 (s, 1H), 7.55 (t, J = 7.7 Hz, 1H), 7.39 (s, 1H), 7.25 (d, J = 8.9 Hz, 1H), 7.15 (d, J = 11.0 Hz, 1H), 6.20 (d, J = 7.1 Hz, 1H), 5.52 (s, 1H), 4.50-4.35 (m, 2H), 3.94-3.83 (m, 5H), 3.19- 3.00 (m, 2H), 2.13 (s, 1H), 1.80 (d, J = 7.2 Hz, 3H), 1.36 (t, J = 7.3 Hz, 3H), 1.12 (d, J = 3.7 Hz, 2H), 0.94-0.82 (m, 2H). | m/z: 570.2 [M + H]⁺ |
| 143 | 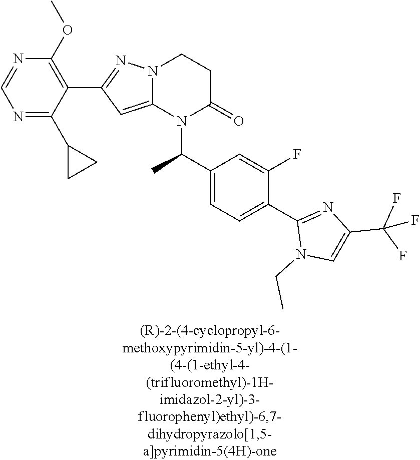 [SMILES: CCn1cc(C(F)(F)F)nc1-c1ccc([C@@H](C)N2C(=O)CCn3nc(-c4c(OC)ncnc4C4CC4)cc32)cc1F] | ¹H NMR (400 MHz, Chloroform-d) δ 8.51 (s, 1H), 7.55 (t, J = 7.7 Hz, 1H), 7.39 (s, 1H), 7.25 (d, J = 8.3 Hz, 1H), 7.15 (d, J = 11.0 Hz, 1H), 6.20 (d, J = 7.1 Hz, 1H), 5.52 (s, 1H), 4.52-4.35 (m, 2H), 3.95-3.81 (m, 5H), 3.19- 2.99 (m, 2H), 2.13 (s, 1H), 1.81 (d, J = 7.1 Hz, 3H), 1.37 (t, J = 7.3 Hz, 3H), 1.12 (d, J = 3.7 Hz, 2H), 0.93-0.84 (m, 2H). | m/z: 570.2 [M + H]⁺ |
| 144 | 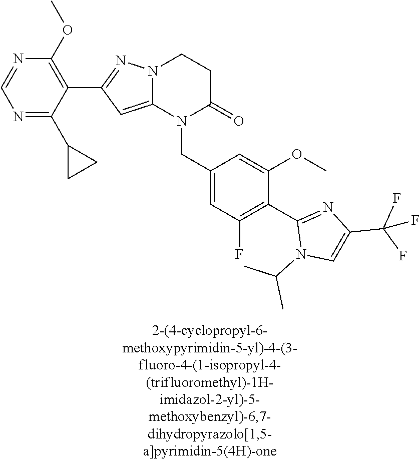 [SMILES: COc1cc(CN2C(=O)CCn3nc(-c4c(OC)ncnc4C4CC4)cc32)cc(F)c1-c1nc(C(F)(F)F)cn1C(C)C] | ¹H NMR (400 MHz, Chloroform-d) δ 8.57 (s, 1H), 7.42 (d, J = 1.3 Hz, 1H), 6.76 (d, J = 8.4 Hz, 2H), 5.88 (s, 1H), 5.09-4.90 (m, 2H), 4.46 (t, J = 7.0 Hz, 2H), 4.11-4.03 (m, 1H), 3.93 (s, 3H), 3.77 (s, 3H), 3.15-3.07 (m, 2H), 2.20 (dt, J = 8.3, 3.8 Hz, 1H), 1.40 (d, J = 6.7 Hz, 3H), 1.35 (d, J = 6.7 Hz, 3H), 1.20-1.16 (m, 2H), 0.94 (dd, J = 8.1, 2.9 Hz, 2H). | m/z: 600.1 [M + H]⁺ |
| 145 | 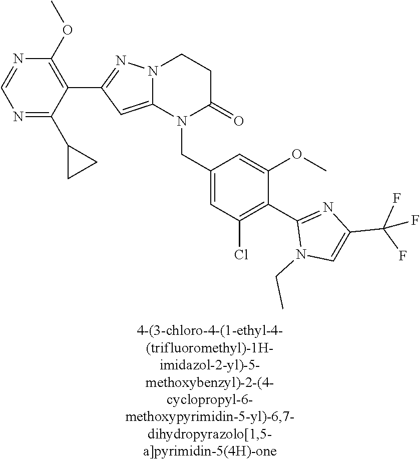 [SMILES: CCn1cc(C(F)(F)F)nc1-c1c(Cl)cc(CN2C(=O)CCn3nc(-c4c(OC)ncnc4C4CC4)cc32)cc1OC] | ¹H NMR (400 MHz, Chloroform-d) δ 8.58 (s, 1H), 7.37 (s, 1H), 7.04 (s, 1H), 6.88 (s, 1H), 5.89 (s, 1H), 4.98 (d, J = 13.1 Hz, 2H), 4.46 (t, J = 7.0 Hz, 2H), 3.94 (s, 3H), 3.74 (d, J = 7.7 Hz, 5H), 3.19-3.07 (m, 2H), 2.24- 2.15 (m, 1H), 1.33 (t, J = 7.3 Hz, 3H), 1.20 (s, 2H), 0.99- 0.92 (m, 2H). | m/z: 602.1 [M + H]⁺ |
| 146 | 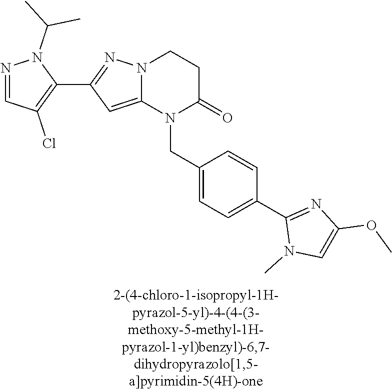 [SMILES: COc1cn(C)c(-c2ccc(CN3C(=O)CCn4nc(-c5c(Cl)cnn5C(C)C)cc43)cc2)n1] | ¹H NMR (400 MHz, Chloroform-d) δ 7.48 (s, 1H), 7.40 (s, 4H), 6.17 (s, 1H), 5.64 (s, 1H), 5.17 (m, 1H), 5.01 (s, 2H), 4.44-4.39 (m, 2H), 3.89 (s, 3H), 3.09 (t, J = 7.1 Hz, 2H), 2.26 (s, 3H), 1.44 (d, J = 6.6 Hz, 6H). | m/z: 480.1 [M + H]⁺ |

### Example 147. 3-chloro-2-(1-isopropyl-4-methyl-1H-pyrazol-5-yl)-4-(4-(1-methyl-4-(trifluoromethyl)-1H-imidazol-2-yl)benzyl)-4,5,6,7-tetrahydropyrazolo[1,5-α]pyrimidine

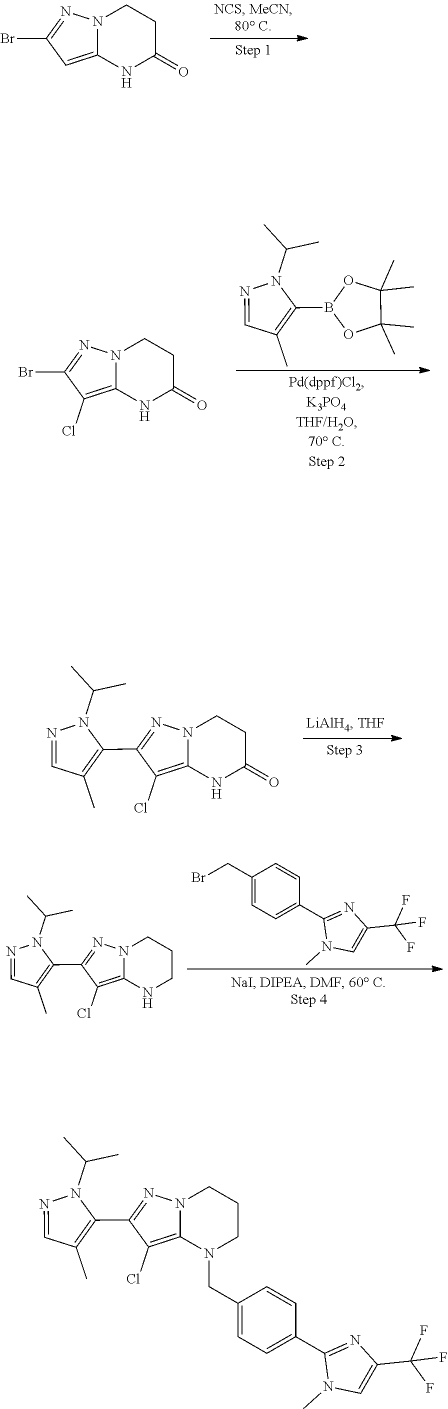

### Step 1. 2-bromo-3-chloro-6,7-dihydropyrazolo[1,5-α]pyrimidin-5(4H)-one

A solution of 2-bromo-6,7-dihydropyrazolo[1,5-α]pyrimidin-5(4H)-one (650 mg, 3.01 mmol) and N-chlorosuccinimide (482.1 mg, 3.61 mmol) in MeCN (15 mL) was heated at 80° C. for 2 h. The reaction mixture was concentrated to dryness and purified by silica gel chromatography (eluting with 5:1 PE/EtOAc) to afford 2-bromo-3-chloro-6,7-dihydropyrazolo[1,5-α]pyrimidin-5(4H)-one (690 mg). MS (ESI) m/z 249.9 [M+H]⁺.

### Step 2. 3-chloro-2-(1-isopropyl-4-methyl-1H-pyrazol-5-yl)-6,7-dihydropyrazolo[1,5-α]pyrimidin-5(4H)-one

3-chloro-2-(1-isopropyl-4-methyl-1H-pyrazol-5-yl)-6,7-dihydropyrazolo[1,5-α]pyrimidin-5(4H)-one was prepared from 2-bromo-3-chloro-6,7-dihydropyrazolo[1,5-α]pyrimidin-5(4H)-one following step 1 of Example 52. MS (ESI) m/z 294.1 [M+H]⁺.

### Step 3. 3-chloro-2-(1-isopropyl-4-methyl-1H-pyrazol-5-yl)-4,5,6,7-tetrahydropyrazolo[1,5-α]pyrimidine

3-chloro-2-(1-isopropyl-4-methyl-1H-pyrazol-5-yl)-4,5,6,7-tetrahydropyrazolo[1,5-α]pyrimidine was prepared from 3-chloro-2-(1-isopropyl-4-methyl-1H-pyrazol-5-yl)-6,7-dihydropyrazolo[1,5-α]pyrimidin-5(4H)-one following step 2 of Example 52. MS (ESI) m/z 280.1 [M+H]⁺.

### Step 4. 3-chloro-2-(1-isopropyl-4-methyl-1H-pyrazol-5-yl)-4-(4-(1-methyl-4-(trifluoromethyl)-1H-imidazol-2-yl) benzyl)-4,5,6,7-tetrahydropyrazolo[1,5-α]pyrimidine

Example 147 3-chloro-2-(1-isopropyl-4-methyl-1H-pyrazol-5-yl)-4-(4-(1-methyl-4-(trifluoromethyl)-1H-imidazol-2-yl)benzyl)-4,5,6,7-tetrahydropyrazolo[1,5-α]pyrimidine was prepared from 3-chloro-2-(1-isopropyl-4-methyl-1H-pyrazol-5-yl)-4,5,6,7-tetrahydropyrazolo[1,5-α]pyrimidine according to step 3 of Example 52.

Table 11. The compounds listed in Table 11 were synthesized according to Example 147 using the appropriate commercially available reagents and/or intermediates described above.

TABLE 11

| Example | | | |
|---|---|---|---|
| number | Structure | ¹H NMR (δ ppm) | MS (ESI) |
| 147 | 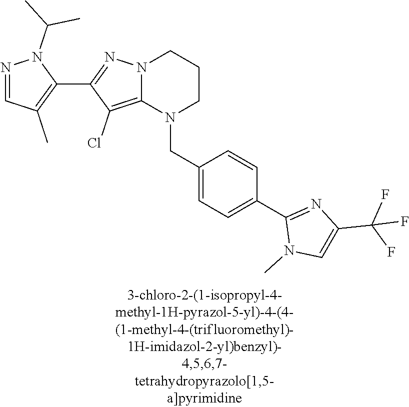 [SMILES: Cc1cnn(C(C)C)c1-c1nn2c(c1Cl)N(Cc1ccc(-c3nc(C(F)(F)F)cn3C)cc1)CCC2] | ¹H NMR (400 MHz, Chloroform-d) δ 7.63 (d, J = 7.6 Hz, 2H), 7.49 (d, J = 7.7 Hz, 2H), 7.42 (s, 1H), 7.32 (s, 1H), 4.77 (s, 2H), 4.52-4.45 (m, 1H), 4.13 (t, J = 5.8 Hz, 2H), 3.78 (s, 3H), 3.16 (t, J = 5.4 Hz, 2H), 2.17-2.11 (m, 2H), 2.03 (s, 3H), 1.45 (d, J = 6.5 Hz, 6H). | m/z: 518.2 [M + H]⁺ |
| 148 | 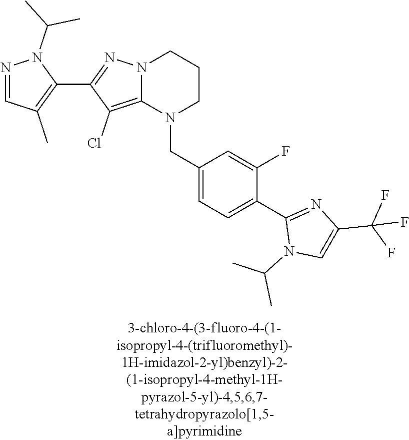 [SMILES: Cc1cnn(C(C)C)c1-c1nn2c(c1Cl)N(Cc1ccc(-c3nc(C(F)(F)F)cn3C(C)C)c(F)c1)CCC2] | ¹H NMR (400 MHz, Chloroform-d) δ 7.55 (t, J = 7.5 Hz, 1H), 7.45 (d, J = 1.3 Hz, 1H), 7.43 (s, 1H), 7.28 (dd, J = 7.8, 1.5 Hz, 1H), 7.24 (dd, J = 10.4, 1.4 Hz, 1H), 4.76 (s, 2H), 4.50-4.44 (m, 1H), 4.33-4.27 (m, 1H), 4.15 (t, J = 6.1 Hz, 2H), 3.20-3.16 (m, 2H), 2.21-2.14 (h, J = 5.9, 5.0 Hz, 2H), 2.03 (s, 3H), 1.44 (t, J = 6.5 Hz, 12H). | m/z: 564.3 [M + H]⁺ |

### Example 149. 4-(3-fluoro-4-(1-methyl-4-(trifluoromethyl)-1H-imidazol-2-yl)benzyl)-2-(4-methoxy-6-(1-methylcyclopropyl)pyrimidin-5-yl)-6,7-dihydropyrazolo[1,5-α]pyrimidin-5(4H)-one

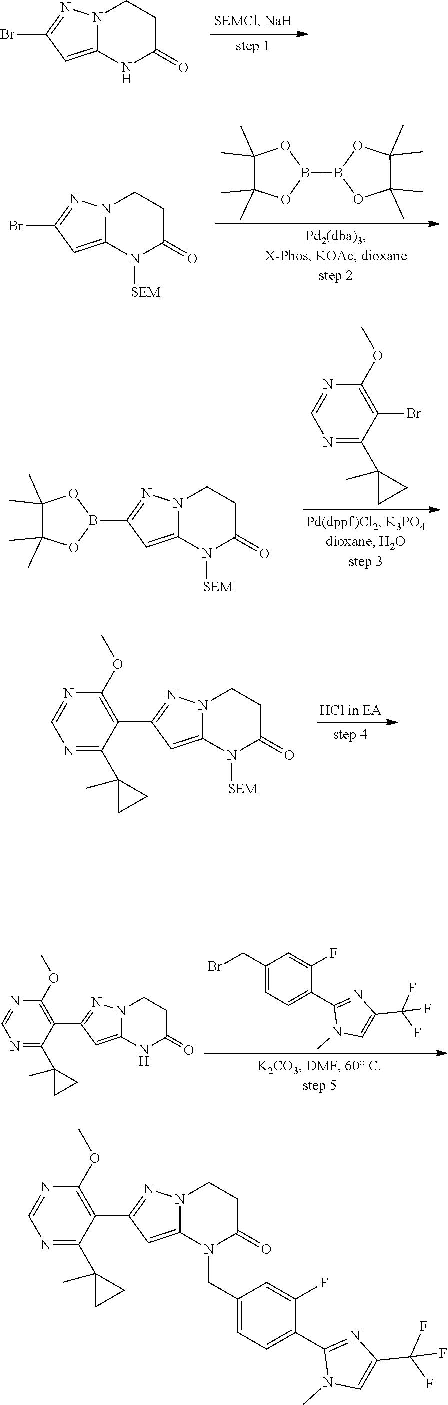

### Step 1. 2-bromo-4-((2-(trimethylsityl)ethoxy)methyl)-6,7-dihydropyrazolo[1,5-α]pyrimidin-5(4H)-one

To a solution of 2-bromo-6,7-dihydropyrazolo[1,5-α]pyrimidin-5(4H)-one (10 g, 46 mmol) in DMF (50 mL) was added NaH (60% in mineral oil, 2.8 g, 69 mmol) at 0° C. The mixture was stirred for 30 min then 2-(Trimethylsilyl)ethoxymethyl chloride (12 mL, 69 mmol) was added. The resulting mixture was stirred at room temperature for 2 h then quenched with water (100 mL) and extracted with EtOAc (100 mL×3). The combined organic phases were washed with brine, dried over anhydrous Na₂SO₄, concentrated and purified by column chromatography on silica gel (eluting with 1/5 EtOAc/PE) to afford 2-bromo-4-((2-(trimethylsilyl)ethoxy)methyl)-6,7-dihydropyrazolo[1,5-α]pyrimidin-5(4H)-one (6.1 g). MS (ESI) m/z 346.1 [M+H]⁺.

### Step 2. 2-(4,4,5,5-tetramethyl-1,3,2-dioxaborolan-2-yl)-4-((2-(trimethylsilyl)ethoxy)methyl)-6,7-dihydropyrazolo [1,5-α]pyrimidin-5(4H)-one

2-(4,4,5,5-tetramethyl-1,3,2-dioxaborolan-2-yl)-4-((2-(trimethylsilyl)ethoxy)methyl)-6,7-dihydropyrazolo[1,5-α]pyrimidin-5(4H)-one was prepared from 2-bromo-4-((2-(trimethylsilyl)ethoxy)methyl)-6,7-dihydropyrazolo[1,5-α]pyrimidin-5(4H)-one following step 2 of Example 154. MS (ESI) m/z 394.2 [M+H]⁺.

### Step 3. 2-(4-methoxy-6-(1-methylcyclopropyl)pyrimidin-5-yl)-4-((2-(trimethylsilyl)ethoxy)methyl)-6,7-dihydropyrazolo[1,5-α]pyrimidin-5(4H)-one

To a solution of 5-bromo-4-methoxy-6-(1-methylcyclopropyl)pyrimidine (Intermediate 13, 200 mg, 0.83 mmol) in dioxane (10 mL) and H₂O (1 mL) was added 2-(4,4,5,5-tetramethyl-1,3,2-dioxaborolan-2-yl)-4-((2-(trimethylsilyl)ethoxy)methyl)-6,7-dihydropyrazolo[1,5-α]pyrimidin-5(4H)-one (258 mg, 0.83 mmol), K₃PO₄ (352 mg, 1.66 mmol) and Pd(dppf)Cl₂ (61 mg, 0.083 mmol). The resulting solution was heated at 100° C. under an atmosphere of N₂ for 1 h. The reaction mixture was filtered through celite, concentrated and purified by prep-HPLC (Conditions 3) to afford 2-(4-methoxy-6-(1-methylcyclopropyl)pyrimidin-5-yl)-4-((2-(trimethylsilyl)ethoxy)methyl)-6,7-dihydropyrazolo[1,5-α]pyrimidin-5(4H)-one (100 mg). MS (ESI) m/z 430.2 [M+H]⁺.

### Step 4. 2-(4-methoxy-6-(1-methylcyclopropyl)pyrimidin-5-yl)-6,7-dihydropyrazolo[1,5-α]pyrimidin-5(4H)-one

A mixture of 2-(4-methoxy-6-(1-methylcyclopropyl)pyrimidin-5-yl)-4-((2-(trimethylsilyl)ethoxy)methyl)-6,7-dihydropyrazolo[1,5-α]pyrimidin-5(4H)-one (350 mg, 0.82 mmol) in HCl EtOAc solution (4M, 10 mL) was stirred at room temperature for 12 h. The pH of the resulting mixture was adjusted to 8 with saturated Na₂CO₃ solution, extracted with EtOAc (15 mL×3), dried over anhydrous Na₂SO₄ and concentrated under vacuum to afford 2-(4-methoxy-6-(1-methylcyclopropyl)pyrimidin-5-yl)-6,7-dihydropyrazolo[1,5-α]pyrimidin-5(4H)-one (189 mg). MS (ESI) m/z 300.2 [M+H]⁺.

### Step 5. 4-(3-fluoro-4-(1-methyl-4-(trifluoromethyl)-1H-imidazol-2-yl)benzyl)-2-(4-methoxy-6-(1-methylcyclopropyl) pyrimidin-5-yl)-6,7-dihydropyrazolo[1,5-α]pyrimidin-5(4H)-one

Example 149 4-(3-fluoro-4-(1-methyl-4-(trifluoromethyl)-1H-imidazol-2-yl)benzyl)-2-(4-methoxy-6-(1-methylcyclopropyl)pyrimidin-5-yl)-6,7-dihydropyrazolo[1,5-α]pyrimidin-5(4H)-one was prepared from 2-(4-methoxy-6-(1-methylcyclopropyl)pyrimidin-5-yl)-6,7-dihydropyrazolo[1,5-α]pyrimidin-5(4H)-one following step 2 of Example 1.

Table 12. The compounds listed in Table 12 were synthesized according to Example 149 using the appropriate commercially available reagents and/or intermediates described above.

TABLE 12

| Example | | | |
|---|---|---|---|
| number | Structure | ¹H NMR (δ ppm) | MS (ESI) |
| 149 | 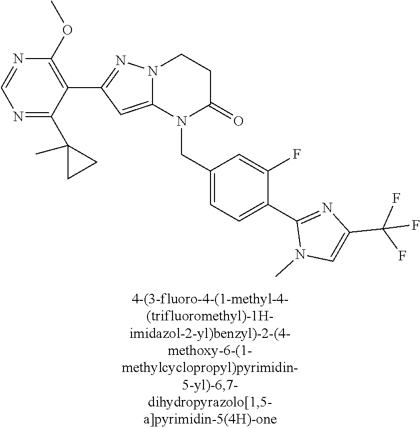 [SMILES: COc1ncnc(C2(C)CC2)c1-c1cc2n(n1)CCC(=O)N2Cc1ccc(-c2nc(C(F)(F)F)cn2C)c(F)c1] | ¹H NMR (400 MHz, Methanol-d₄) δ 8.64 (s, 1H), 7.75 (q, J = 1.2 Hz, 1H), 7.54 (t, J = 7.6 Hz, 1H), 7.40-7.32 (m, 2H), 5.99 (s, 1H), 5.14 (s, 2H), 4.47 (t, J = 7.1 Hz, 2H), 3.92 (s, 3H), 3.64 (d, J = 1.7 Hz, 3H), 3.17 (t, J = 7.1 Hz, 2H), 1.27 (s, 3H), 0.72-0.66 (m, 2H), 0.48-0.43 (m, 2H). | m/z: 556.3 [M + H]⁺ |
| 150 | 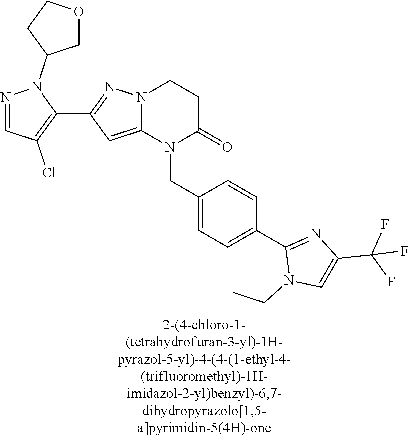 [SMILES: CCn1cc(C(F)(F)F)nc1-c1ccc(CN2C(=O)CCn3nc(-c4c(Cl)cnn4C4CCOC4)cc32)cc1] | ¹H NMR (400 MHz, Chloroform-d) δ 7.55 (s, 2H), 7.49 (s, 1H), 7.44 (s, 2H), 7.36 (s, 1H), 6.19 (s, 1H), 5.66 (s, 1H), 5.13-4.98 (m, 2H), 4.43 (s, 2H), 4.17 (d, J = 7.6 Hz, 1H), 4.15-4.02 (m, 3H), 3.98 (s, 2H), 3.11 (s, 2H), 2.48 (s, 1H), 2.33 (s, 1H), 1.43 (s, 3H). | m/z: 560.2 [M + H]⁺ |
| 151 | 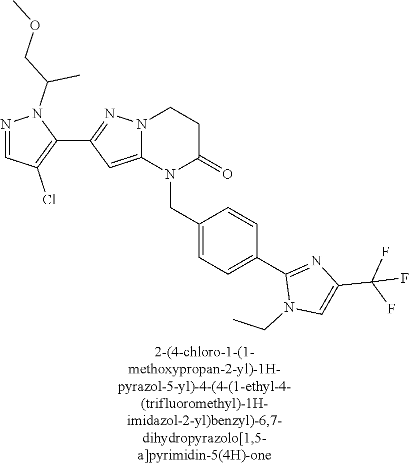 [SMILES: CCn1cc(C(F)(F)F)nc1-c1ccc(CN2C(=O)CCn3nc(-c4c(Cl)cnn4C(C)COC)cc32)cc1] | ¹H NMR (400 MHz, Chloroform-d) δ 7.54 (d, J = 8.1 Hz, 2H), 7.49 (s, 1H), 7.42 (d, J = 8.1 Hz, 2H), 7.35 (s, 1H), 6.19 (s, 1H), 5.29-5.21 (m, 1H), 5.03 (s, 2H), 4.41 (t, J = 7.1 Hz, 2H), 4.03 (q, J = 7.3 Hz, 2H), 3.82 (dd, J = 9.7, 7.9 Hz, 1H), 3.56 (dd, J = 9.8, 5.6 Hz, 1H), 3.24 (s, 3H), 3.08 (t, J = 7.1 Hz, 2H), 1.45-1.38 (m, 6H). | m/z: 562.3 [M + H]⁺ |
| 152 | 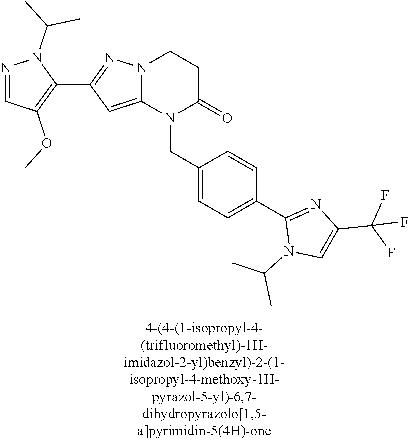 [SMILES: COc1cnn(C(C)C)c1-c1cc2n(n1)CCC(=O)N2Cc1ccc(-c2nc(C(F)(F)F)cn2C(C)C)cc1] | ¹H NMR (400 MHz, Methanol-d₄) δ 7.90 (s, 1H), 7.54 (s, 4H), 7.35 (s, 1H), 6.19 (s, 1H), 5.39-5.33 (m, 1H), 5.12 (s, 2H), 4.57-4.50 (m, 1H), 4.44 (t, J = 7.0 Hz, 2H), 3.76 (s, 3H), 3.13 (t, J = 7.0 Hz, 2H), 1.44 (d, J = 6.7 Hz, 6H), 1.39 (d, J = 6.6 Hz, 6H). | m/z: 542.3 [M + H]⁺ |
| 153 | 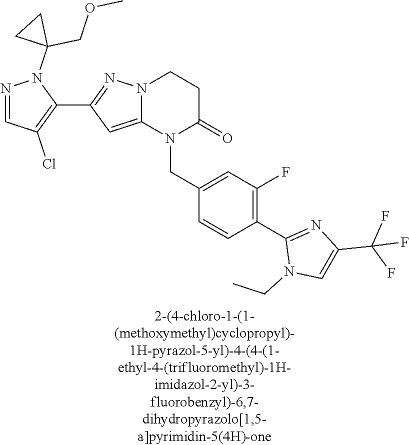 [SMILES: CCn1cc(C(F)(F)F)nc1-c1ccc(CN2C(=O)CCn3nc(-c4c(Cl)cnn4C4(COC)CC4)cc32)cc1F] | ¹H NMR (400 MHz, Chloroform-d) δ 7.55 (t, J = 7.6 Hz, 1H), 7.46 (s, 1H), 7.40 (s, 1H), 7.27 (d, J = 5.7 Hz, 1H), 7.21-7.12 (m, 1H), 6.32 (s, 1H), 5.03 (s, 2H), 4.45 (t, J = 7.1 Hz, 2H), 3.90 (q, J = 7.3 Hz, 2H), 3.69 (s, 2H), 3.28 (s, 3H), 3.12 (t, J = 7.1 Hz, 2H), 1.38 (t, J = 7.3 Hz, 3H), 1.11 (d, J = 2.2 Hz, 2H), 0.98 (d, J = 2.3 Hz, 2H). | m/z: 592.2 [M + H]⁺ |

### Example 154. 2-(4-chloro-1-(oxetan-3-yl)-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one

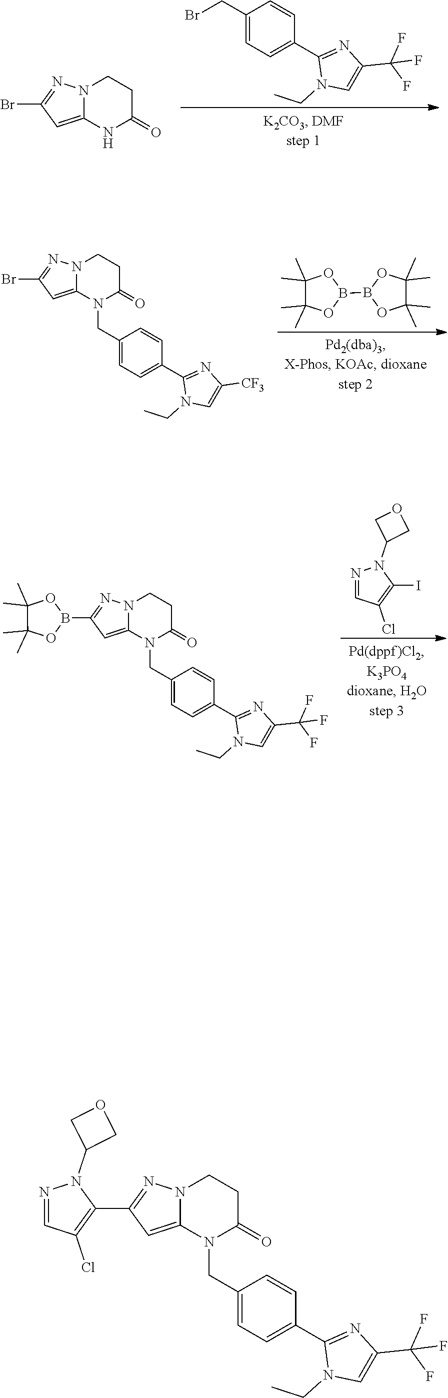

### Step 1. 2-bromo-4-(4-(1-ethyl-4-(trifluoromethyl)-1H-imidazol-2-yl)benzyl)-6,7-dihydropyrazolo[1,5-α]pyrimidin-5(4H)-one

2-bromo-4-(4-(1-ethyl-4-(trifluoromethyl)-1H-imidazol-2-yl)benzyl)-6,7-dihydropyrazolo[1,5-α]pyrimidin-5(4H)-one was prepared from 2-bromo-6,7-dihydropyrazolo[1,5-α]pyrimidin-5(4H)-one and 2-(4-(bromomethyl)phenyl)-1-ethyl-4-(trifluoromethyl)-1H-imidazole according to step 2 of Example 1. MS (ESI) m/z 468.1

### Step 2. 4-(4-(1-ethyl-4-(trifluoromethyl)-1H-imidazol-2-yl)benzyl)-2-(4,4,5,5-tetramethyl-1,3,2-dioxaborolan-2-yl)-6,7-dihydropyrazolo[1,5-α]pyrimidin-5(4H)-one

A solution of 2-bromo-4-(4-(1-ethyl-4-(trifluoromethyl)-1H-imidazol-2-yl)benzyl)-6,7-dihydropyrazolo[1,5-α]pyrimidin-5(4H)-one (11 g, 23.5 mmol, 1.0 eq), 4,4,4′,4′,5,5,5′,5′-octamethyl-2,2′-bi(1,3,2-dioxaborolane) (17.9 g, 70.5 mmol, 3.0 eq) , Pd₂(dba)₃ (2.15 g, 2.4 mmol, 0.1 eq), x-Phos (2.24 g, 4.7 mmol, 0.2 eq) and KOAc (6.9 g, 70.5 mmol, 3.0 eq) in dioxane (110 mL) was heated at 90° C. for 4 h under N₂. The solution was filtered through celite and the filtrate concentrated under vacuum. The crude product was purified by silica gel chromatography (eluting with 5:1 DCM/MeOH) to afford the product 4-(4-(1-ethyl-4-(trifluoromethyl)-1H-imidazol-2-yl)benzyl)-2-(4,4,5,5-tetramethyl-1,3,2-dioxaborolan-2-yl)-6,7-dihydropyrazolo[1,5-α]pyrimidin-5(4H)-one (5.6 g). MS (ESI) m/z 434.1 [M+H]⁺, as the MS of boric acid.

### Step 3. 2-(4-chloro-1-(oxetan-3-yl)-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one

Example 154 2-(4-chloro-1-(oxetan-3-yl)-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one was prepared from 4-(4-(1-ethyl-4-(trifluoromethyl)-1H-imidazol-2-yl)benzyl)-2-(4,4,5,5-tetramethyl-1,3,2-dioxaborolan-2-yl)-6,7-dihydropyrazolo[1,5-α]pyrimidin-5(4H)-one following step 3 of Example 149.

Table 13. The compounds listed in Table 13 were synthesized according to Example 154 using the appropriate commercially available reagents and/or intermediates described above.

TABLE 13

| Example | | | |
|---|---|---|---|
| number | Structure | ¹H NMR (δ ppm) | MS (ESI) |
| 154 | 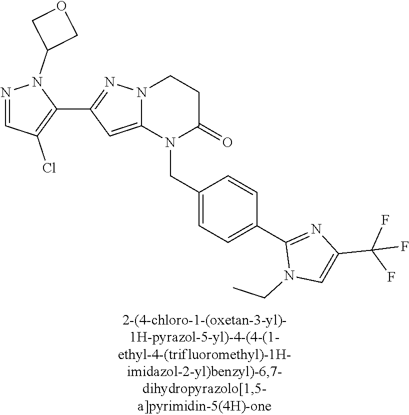 [SMILES: CCn1cc(C(F)(F)F)nc1-c1ccc(CN2C(=O)CCn3nc(-c4c(Cl)cnn4C4COC4)cc32)cc1] | ¹H NMR (400 MHz, Chloroform-d) δ 7.59-7.52 (m, 3H), 7.42 (d, J = 8.1 Hz, 2H), 7.35 (s, 1H), 6.19 (s, 1H), 6.09-6.02 (m, 1H), 5.15 (t, J = 6.5 Hz, 2H), 5.04 (s, 2H), 4.94 (t, J = 7.2 Hz, 2H), 4.42 (t, J = 7.1 Hz, 2H), 4.04 (q, J = 7.3 Hz, 2H), 3.10 (t, J = 7.1 Hz, 2H), 1.42 (t, J = 7.3 Hz, 3H). | m/z: 546.2 [M + H]⁺ |
| 155 | 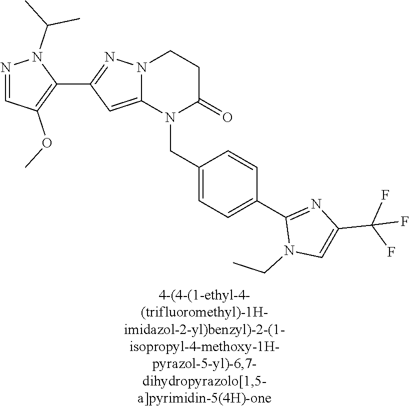 [SMILES: CCn1cc(C(F)(F)F)nc1-c1ccc(CN2C(=O)CCn3nc(-c4c(OC)cnn4C(C)C)cc32)cc1] | ¹H NMR (400 MHz, Chloroform-d) δ 7.54 (d, J = 8.0 Hz, 2H), 7.43 (d, J = 8.0 Hz, 2H), 7.35 (s, 1H), 7.30 (s, 1H), 6.12 (s, 1H), 5.34-5.26 (m, 1H), 5.03 (s, 2H), 4.40 (t, J = 7.1 Hz, 2H), 4.04 (q, J = 7.3 Hz, 2H), 3.80 (s, 3H), 3.07 (t, J = 7.1 Hz, 2H), 1.43 (dd, J = 13.2, 6.9 Hz, 9H). | m/z: 528.2 [M + H]⁺ |
| 156 | 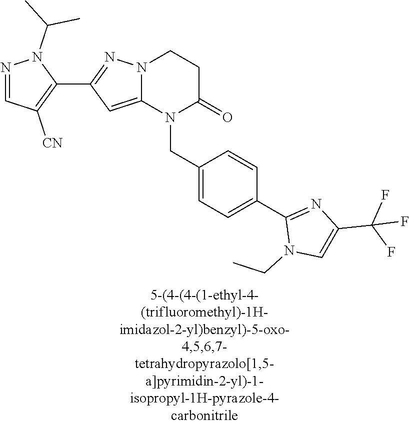 [SMILES: CCn1cc(C(F)(F)F)nc1-c1ccc(CN2C(=O)CCn3nc(-c4c(C#N)cnn4C(C)C)cc32)cc1] | ¹H NMR (400 MHz, Chloroform-d) δ 7.79 (s, 1H), 7.56 (d, J = 7.9 Hz, 2H), 7.45 (d, J = 7.9 Hz, 2H), 7.35 (s, 1H), 6.31 (s, 1H), 5.42-5.36 (m, 1H), 5.05 (s, 2H), 4.43 (t, J = 7.0 Hz, 2H), 4.05 (q, J = 7.2 Hz, 2H), 3.11 (t, J = 7.0 Hz, 2H), 1.48 (d, J = 6.5 Hz, 6H), 1.42 (t, J = 7.2 Hz, 3H). | m/z: 523.2 [M + H]⁺ |
| 157 | 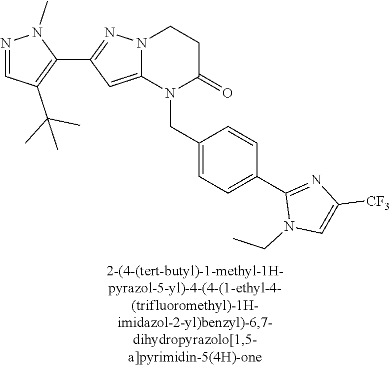 [SMILES: CCn1cc(C(F)(F)F)nc1-c1ccc(CN2C(=O)CCn3nc(-c4c(C(C)(C)C)cnn4C)cc32)cc1] | ¹H NMR (400 MHz, Chloroform-d) δ 7.54 (d, J = 7.9 Hz, 2H), 7.40 (d, J = 7.8 Hz, 3H), 7.36 (d, J = 1.5 Hz, 1H), 5.76 (s, 1H), 5.03 (s, 2H), 4.44 (t, J = 7.1 Hz, 2H), 4.03 (q, J = 7.3 Hz, 2H), 3.62 (s, 3H), 3.12 (t, J = 7.1 Hz, 2H), 1.42 (t, J = 7.3 Hz, 3H), 1.13 (s, 9H). | m/z: 526.3 [M + H]⁺ |
| 158 | 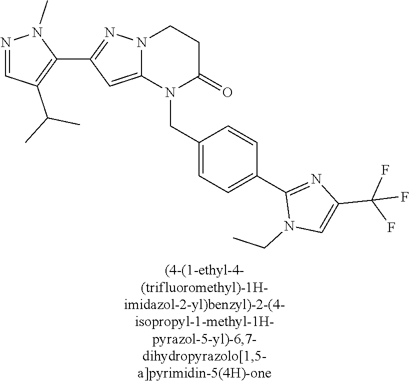 [SMILES: CCn1cc(C(F)(F)F)nc1-c1ccc(CN2C(=O)CCn3nc(-c4c(C(C)C)cnn4C)cc32)cc1] | ¹H NMR (400 MHz, Chloroform-d) δ 7.56 (d, J = 7.8 Hz, 2H), 7.44-7.34 (m, 4H), 5.78 (s, 1H), 5.04 (s, 2H), 4.43 (t, J = 7.1 Hz, 2H), 4.04 (q, J = 7.3 Hz, 2H), 3.88 (s, 3H), 3.11 (t, J = 7.1 Hz, 2H), 2.90-2.80 (m, 1H), 1.42 (t, J = 7.3 Hz, 3H), 1.15 (d, J = 6.9 Hz, 6H). | m/z: 512.2 [M + H]⁺ |
| 159 | 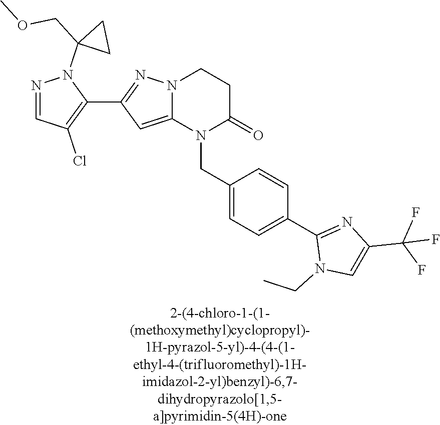 [SMILES: CCn1cc(C(F)(F)F)nc1-c1ccc(CN2C(=O)CCn3nc(-c4c(Cl)cnn4C4(COC)CC4)cc32)cc1] | ¹H NMR (400 MHz, Chloroform-d) δ 7.56 (d, J = 7.0 Hz, 2H), 7.45 (d, J = 4.6 Hz, 3H), 7.35 (s, 1H), 6.30 (s, 1H), 5.04 (s, 2H), 4.44 (t, J = 6.8 Hz, 2H), 4.04 (q, J = 6.7 Hz, 2H), 3.68 (s, 2H), 3.27 (s, 3H), 3.11 (t, J = 6.8 Hz, 2H), 1.42 (t, J = 6.4 Hz, 3H), 1.10 (d, J = 5.9 Hz, 2H), 0.98 (d, J = 6.0 Hz, 2H). | m/z: 574.2 [M + H]⁺ |
| 160 | 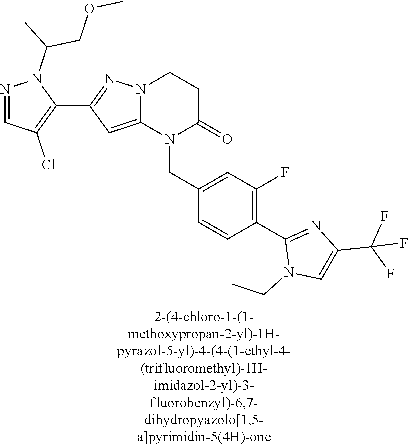 [SMILES: CCn1cc(C(F)(F)F)nc1-c1ccc(CN2C(=O)CCn3nc(-c4c(Cl)cnn4C(C)COC)cc32)cc1F] | ¹H NMR (400 MHz, Chloroform-d) δ 7.59-7.49 (m, 2H), 7.41-7.38 (m, 1H), 7.26-7.23 (m, 1H), 7.15 (d, J = 10.4 Hz, 1H), 6.19 (s, 1H), 5.26 (d, J = 6.4 Hz, 1H), 5.03 (s, 2H), 4.44 (t, J = 7.1 Hz, 2H), 3.94-3.79 (m, 3H), 3.58 (dd, J = 9.8, 5.6 Hz, 1H), 3.26 (s, 3H), 3.10 (t, J = 7.1 Hz, 2H), 1.48-1.36 (m, 6H). | m/z: 580.2 [M + H]⁺ |
| 161 | 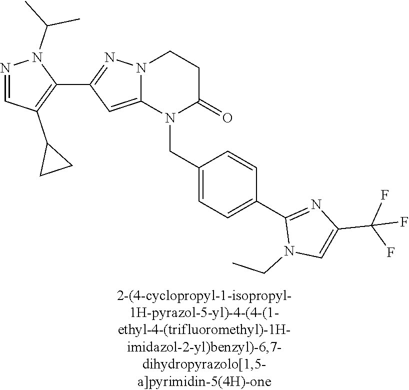 [SMILES: CCn1cc(C(F)(F)F)nc1-c1ccc(CN2C(=O)CCn3nc(-c4c(C5CC5)cnn4C(C)C)cc32)cc1] | ¹H NMR (400 MHz, Chloroform-d) δ 7.55 (d, J = 8.0 Hz, 2H), 7.42 (d, J = 8.0 Hz, 2H), 7.35 (s, 1H), 7.18 (s, 1H), 6.01 (s, 1H), 5.09-4.96 (m, 3H), 4.44 (t, J = 7.0 Hz, 2H), 4.04 (q, J = 7.2 Hz, 2H), 3.11 (t, J = 7.0 Hz, 2H), 1.58 (dq, J = 8.1, 4.1, 3.0 Hz, 1H), 1.43 (d, J = 6.8 Hz, 9H), 0.73 (d, J = 6.8 Hz, 2H), 0.52 (d, J = 4.3 Hz, 2H). | m/z: 538.2 [M + H]⁺ |
| 162 | 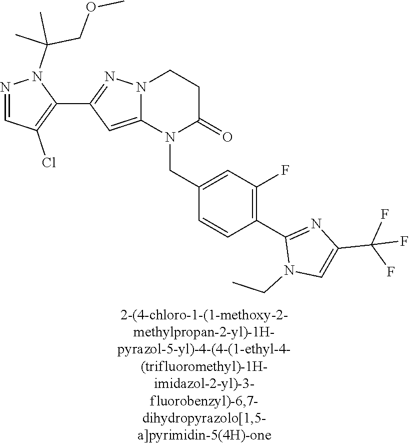 [SMILES: CCn1cc(C(F)(F)F)nc1-c1ccc(CN2C(=O)CCn3nc(-c4c(Cl)cnn4C(C)(C)COC)cc32)cc1F] | ¹H NMR (400 MHz, Chloroform-d) δ 7.54 (t, J = 7.6 Hz, 1H), 7.45 (s, 1H), 7.40 (d, J = 1.2 Hz, 1H), 7.22 (dd, J = 7.9, 1.7 Hz, 1H), 7.13 (dd, J = 10.4, 1.6 Hz, 1H), 5.84 (s, 1H), 5.02 (s, 2H), 4.46 (t, J = 7.1 Hz, 2H), 3.88 (q, J = 7.3 Hz, 2H), 3.59 (s, 2H), 3.20 (s, 3H), 3.12 (t, J = 7.1 Hz, 2H), 1.46 (s, 6H), 1.37 (t, J = 7.3 Hz, 3H). | m/z: 594.2 [M + H]⁺ |
| 163 | 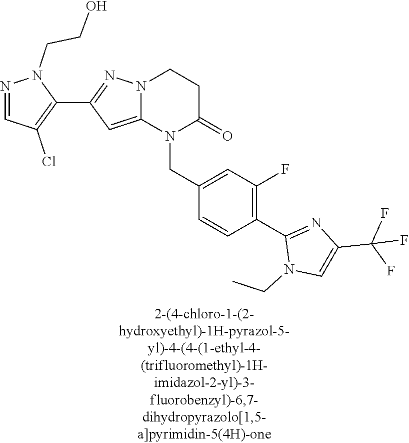 [SMILES: CCn1cc(C(F)(F)F)nc1-c1ccc(CN2C(=O)CCn3nc(-c4c(Cl)cnn4CCO)cc32)cc1F] | ¹H NMR (400 MHz, Chloroform-d) δ 7.55 (t, J = 7.6 Hz, 1H), 7.50 (s, 1H), 7.40 (s, 1H), 7.23 (d, J = 8.0 Hz, 1H), 7.14 (d, J = 10.3 Hz, 1H), 6.20 (s, 1H), 5.04 (s, 2H), 4.55 (t, J = 4.8 Hz, 2H), 4.44 (t, J = 7.1 Hz, 2H), 4.02 (t, J = 4.9 Hz, 2H), 3.90 (q, J = 7.3 Hz, 2H), 3.11 (t, J = 7.1 Hz, 2H), 1.38 (t, J = 7.3 Hz, 3H). | m/z: 552.0 [M + H]⁺ |

### Example 164. 2-(4-cyclopropyl-6-methoxypyrimidin-5-yl)-4-(3-fluoro-4-(1-isopropyl (trifluoromethyl)-1H-imidazol-2-yl)benzyl)-6,7-dihydro-[1,2,4]triazolo[1,5-α]pyrimidin-5(4H)-one

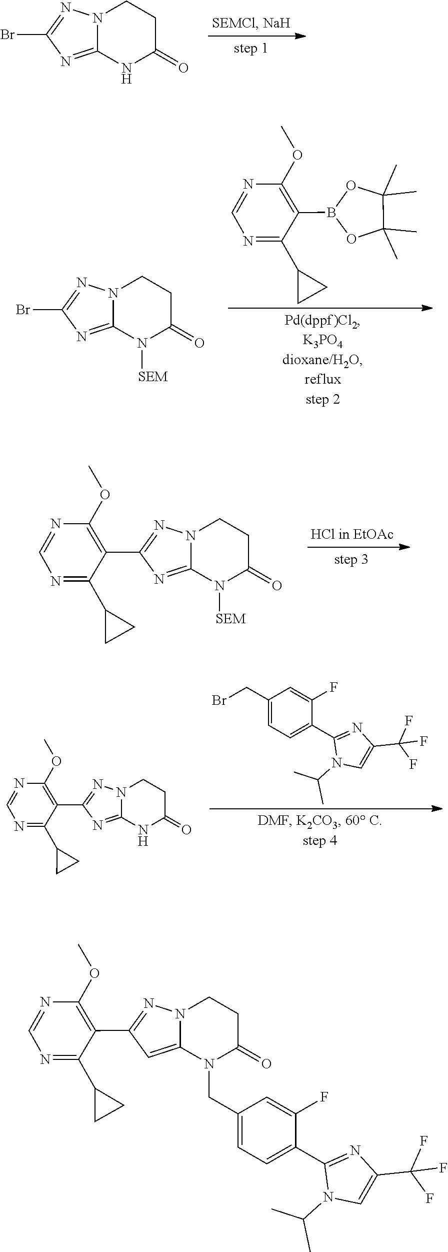

### Step 1. 2-bromo-4-((2-(trimethylsilyl)ethoxy)methyl)-6,7-dihydro-[1,2,4]triazolo[1,5-α]pyrimidin-5(4H)-one

2-bromo-4-((2-(trimethylsilyl)ethoxy)methyl)-6,7-dihydro-[1,2,4]triazolo[1,5-α]pyrimidin-5(4H)-one was prepared from 2-bromo-6,7-dihydro-[1,2,4]triazolo[1,5-α]pyrimidin-5(4H)-one (prepared in step 2 of Example 37) according to step 1 of Example 149. MS (ESI) m/z 347.0, 349.0 [M+H]⁺.

### Step 2. 2-(4-cyclopropyl-6-methoxypyrimidin-5-yl)-4-((2-(trimethylsilyl)ethoxy)methyl)-6,7-dihydro-[1,2,4]triazolo[1,5-α]pyrimidin-5(4H)-one

2-(4-cyclopropyl-6-methoxypyrimidin-5-yl)-4-((2-(trimethylsilyl)ethoxy)methyl)-6,7-dihydro-[1,2,4]triazolo[1,5-α]pyrimidin-5(4H)-one was prepared from 2-bromo-4-((2-(trimethylsilyl)ethoxy)methyl)-6,7-dihydro-[1,2,4]triazolo [1,5-α]pyrimidin-5(4H)-one according to step 3 of Example 1. MS (ESI) m/z 417.2 [M+H]⁺.

### Step 3. 2-(4-cyclopropyl-6-methoxypyrimidin-5-yl)-6,7-dihydro-[1,2,4]triazolo[1,5-α]pyrimidin-5(4H)-one

2-(4-cyclopropyl-6-methoxypyrimidin-5-yl)-6,7-dihydro-[1,2,4]triazolo[1,5-α]pyrimidin-5(4H)-one was prepared from 2-(4-cyclopropyl-6-methoxypyrimidin-5-yl)-4-((2-(trimethylsilyl)ethoxy)methyl)-6,7-dihydro-[1,2,4]triazolo [1,5-α]pyrimidin-5(4H)-one following step 4 of Example 149. MS (ESI) m/z 287.1 [M+H]⁺.

### Step 4. 2-(4-cyclopropyl-6-methoxypyrimidin-5-yl)-4-(3-fluoro-4-(1-isopropyl-4-(trifluoromethyl)-1H-imidazol-2-yl) benzyl)-6,7-dihydro-[1,2,4]triazolo[1,5-α]pyrimidin-5(4H)-one

Example 164 2-(4-cyclopropyl-6-methoxypyrimidin-5-yl)-4-(3-fluoro-4-(1-isopropyl-4-(trifluoromethyl)-1H-imidazol-2-yl)benzyl)-6,7-dihydro-[1,2,4]triazolo[1,5-α]pyrimidin-5(4H)-one was prepared from 2-(4-cyclopropyl-6-methoxypyrimidin-5-yl)-6,7-dihydro-[1,2,4]triazolo[1,5-α]pyrimidin-5(4H)-one following step 2 of Example 1.

Table 14. The compounds listed in Table 14 were synthesized according to Example 164 using the appropriate commercially available reagents and/or intermediates described above. Enantiomers, when generated, were separated by chiral prep-HPLC and absolute stereochemistries were assigned by comparing retention time on chiral HPLC with similar chiral compounds that were obtained via enantioselective synthesis.

TABLE 14

| Example | | | |
|---|---|---|---|
| number | Structure | ¹H NMR (δ ppm) | MS (ESI) |
| 164 | 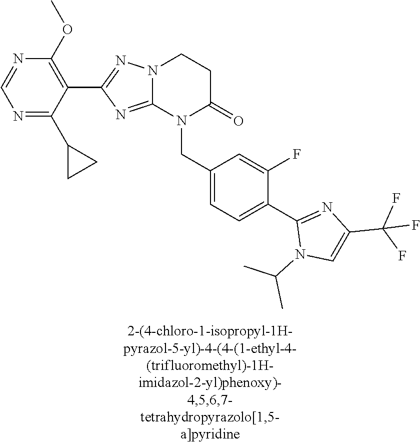 [SMILES: COc1ncnc(C2CC2)c1-c1nc2n(n1)CCC(=O)N2Cc1ccc(-c2nc(C(F)(F)F)cn2C(C)C)c(F)c1] | ¹H NMR (400 MHz, Chloroform-d) δ 8.62 (s, 1H), 7.50-7.39 (m, 4H), 5.19 (s, 2H), 4.41 (t, J = 7.2 Hz, 2H), 4.30-4.20 (m, 1H), 3.97 (s, 3H), 3.12 (t, J = 7.2 Hz, 2H), 2.06-2.01 (m, 1H), 1.40 (d, J = 6.6 Hz, 6H), 1.23 (dd, J = 7.3, 3.5 Hz, 2H), 0.97 (dd, J = 7.7, 2.8 Hz, 2H). | m/z: 571.2 [M + H]⁺ |
| 165 | 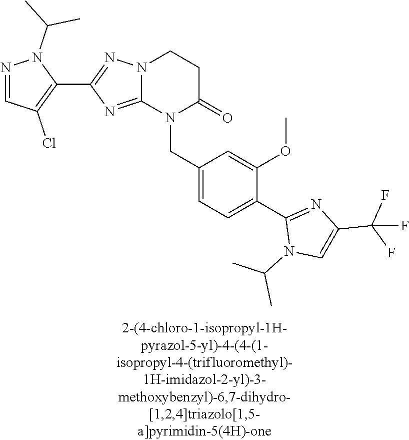 [SMILES: COc1cc(CN2C(=O)CCn3nc(-c4c(Cl)cnn4C(C)C)nc32)ccc1-c1nc(C(F)(F)F)cn1C(C)C] | ¹H NMR (400 MHz, Chloroform-d) δ 7.54 (s, 1H), 7.36 (d, J = 8.3 Hz, 2H), 7.23 (d, J = 7.8 Hz, 1H), 7.18 (s, 1H), 5.28-5.16 (m, 3H), 4.41 (t, J = 7.2 Hz, 2H), 4.19-4.10 (m, 1H), 3.77 (s, 3H), 3.12 (t, J = 7.3 Hz, 2H), 1.49 (d, J = 6.6 Hz, 6H), 1.35 (d, J = 6.6 Hz, 6H). | m/z: 577.2 [M + H]⁺ |
| 166 | 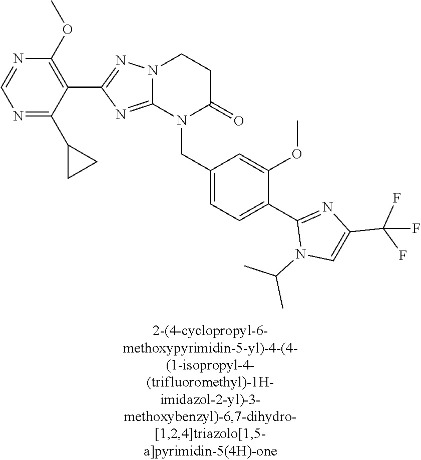 [SMILES: COc1cc(CN2C(=O)CCn3nc(-c4c(OC)ncnc4C4CC4)nc32)ccc1-c1nc(C(F)(F)F)cn1C(C)C] | ¹H NMR (400 MHz, Chloroform-d) δ 8.62 (s, 1H), 7.38-7.52 (m, 2H), 7.24-7.17 (m, 2H), 5.18 (s, 2H), 4.40 (t, J = 7.3 Hz, 2H), 4.17-4.11 (m, 1H), 3.95 (s, 3H), 3.76 (s, 3H), 3.11 (t, J = 7.3 Hz, 2H), 1.99-1.93 (m, 1H), 1.35 (d, J = 6.7 Hz, 6H), 1.22-1.19 (m, 2H), 0.96-0.91 (m, 2H). | m/z: 583.2 [M + H]⁺ |
| 167 | 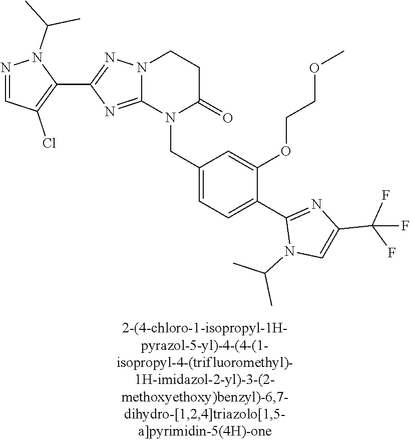 [SMILES: COCCOc1cc(CN2C(=O)CCn3nc(-c4c(Cl)cnn4C(C)C)nc32)ccc1-c1nc(C(F)(F)F)cn1C(C)C] | ¹H NMR (400 MHz, Chloroform-d) δ 7.54 (s, 1H), 7.38-7.34 (m, 2H), 7.24 (s, 1H), 7.21 (s, 1H), 5.27-5.20 (m, 1H), 5.17 (s, 2H), 4.41 (t, J = 7.1 Hz, 2H), 4.25 (t, J = 6.6 Hz, 1H), 4.07 (t, J = 4.5 Hz, 2H), 3.56 (t, J = 4.4 Hz, 2H), 3.25 (s, 3H), 3.12 (t, J = 7.1 Hz, 2H), 1.49 (d, J = 6.5 Hz, 6H), 1.34 (d, J = 6.5 Hz, 6H). | m/z: 621.2 [M + H]⁺ |
| 168 | 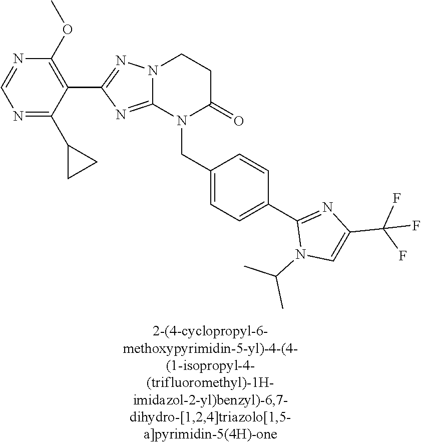 [SMILES: COc1ncnc(C2CC2)c1-c1nc2n(n1)CCC(=O)N2Cc1ccc(-c2nc(C(F)(F)F)cn2C(C)C)cc1] | ¹H NMR (400 MHz, Chloroform-d) δ 8.63 (s, 1H), 7.65 (d, J = 8.2 Hz, 2H), 7.47 (d, J = 8.2 Hz, 2H), 7.42-7.39 (m, 1H), 5.21 (s, 2H), 4.56- 4.49 (m, 1H), 4.41 (t, J = 7.3 Hz, 2H), 3.97 (s, 3H), 3.12 (t, J = 7.3 Hz, 2H), 2.06-1.99 (m, 1H), 1.44 (d, J = 6.7 Hz, 6H), 1.25-1.21 (m, 2H), 0.98-0.93 (m, 2H). | m/z: 553.3 [M + H]⁺ |
| 169 | 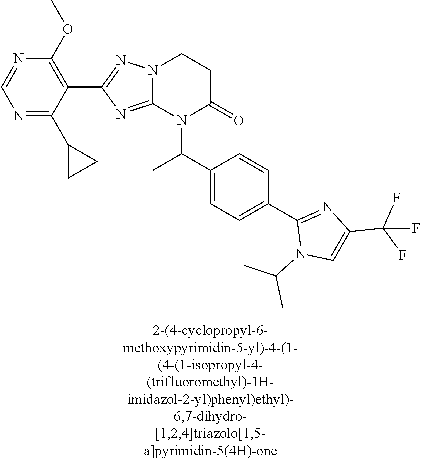 [SMILES: COc1ncnc(C2CC2)c1-c1nc2n(n1)CCC(=O)N2C(C)c1ccc(-c2nc(C(F)(F)F)cn2C(C)C)cc1] | ¹H NMR (400 MHz, Chloroform-d) δ 8.61 (s, 1H), 7.64 (d, J = 8.2 Hz, 2H), 7.47 (d, J = 8.2 Hz, 2H), 7.40 (s, 1H), 6.17 (q, J = 7.1 Hz, 1H), 4.56 (m, 1H), 4.39 (tq, J = 10.0, 5.7 Hz, 2H), 3.96 (s, 3H), 3.08 (t, J = 7.3 Hz, 2H), 2.00 (d, J = 7.4 Hz, 4H), 1.45 (dd, J = 8.7, 6.8 Hz, 6H), 1.20 (dt, J = 7.8, 4.4 Hz, 2H), 0.92 (dd, J = 8.0, 2.9 Hz, 2H). | m/z: 567.2 [M + H]⁺ |
| 170 | 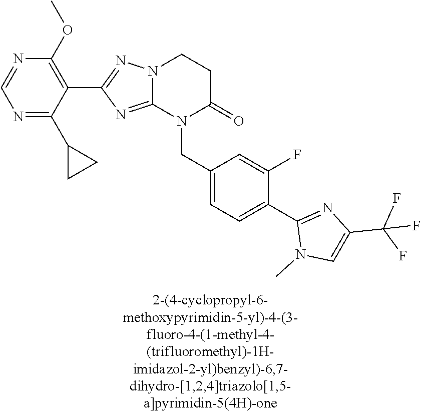 [SMILES: COc1ncnc(C2CC2)c1-c1nc2n(n1)CCC(=O)N2Cc1ccc(-c2nc(C(F)(F)F)cn2C)c(F)c1] | ¹H NMR (400 MHz, Chloroform-d) δ 8.62 (s, 1H), 7.54 (t, J = 7.6 Hz, 1H), 7.47- 7.40 (m, 2H), 7.34 (d, J = 1.3 Hz, 1H), 5.19 (s, 2H), 4.41 (t, J = 7.3 Hz, 2H), 3.96 (s, 3H), 3.62 (d, J = 2.3 Hz, 3H), 3.12 (t, J = 7.3 Hz, 2H), 2.07-1.99 (m, 1H), 1.22 (dd, J = 4.5, 2.8 Hz, 2H), 0.99-0.94 (m, 2H). | m/z: 543.2 [M + H]⁺ |
| 171 | 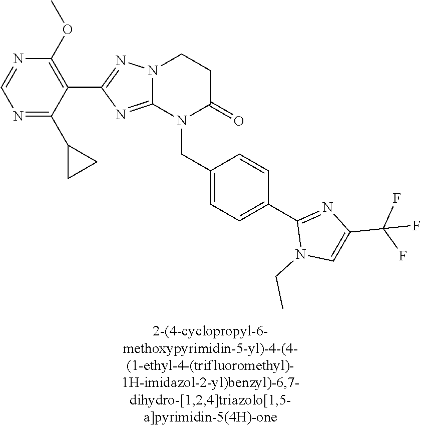 [SMILES: CCn1cc(C(F)(F)F)nc1-c1ccc(CN2C(=O)CCn3nc(-c4c(OC)ncnc4C4CC4)nc32)cc1] | ¹H NMR (400 MHz, Chloroform-d) δ 8.62 (s, 1H), 7.64 (d, J = 8.3 Hz, 2H), 7.53- 7.48 (m, 2H), 7.35 (d, J = 1.1 Hz, 1H), 5.21 (s, 2H), 4.40 (t, J = 7.3 Hz, 2H), 4.04 (q, J = 7.3 Hz, 2H), 3.96 (s, 3H), 3.12 (t, J = 7.3 Hz, 2H), 2.06-1.97 (m, 1H), 1.42 (t, J = 7.3 Hz, 3H), 1.22 (dd, J = 4.5, 2.9 Hz, 2H), 0.94 (dd, J = 8.1, 3.0 Hz, 2H). | m/z: 539.2 [M + H]⁺ |
| 172 | 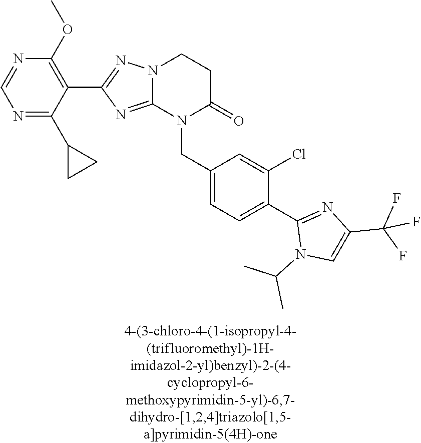 [SMILES: COc1ncnc(C2CC2)c1-c1nc2n(n1)CCC(=O)N2Cc1ccc(-c2nc(C(F)(F)F)cn2C(C)C)c(Cl)c1] | ¹H NMR (400 MHz, Chloroform-d) δ 8.62 (s, 1H), 7.68 (d, J = 1.4 Hz, 1H), 7.55 (dd, J = 7.9, 1.5 Hz, 1H), 7.42-7.40 (m, 1H), 7.38 (d, J = 7.8 Hz, 1H), 5.18 (s, 2H), 4.42 (t, J = 7.3 Hz, 2H), 4.11 (dt, J = 13.3, 6.7 Hz, 1H), 3.97 (s, 3H), 3.12 (t, J = 7.3 Hz, 2H), 2.03 (tt, J = 8.2, 4.7 Hz, 1H), 1.38 (d, J = 6.1 Hz, 6H), 1.22 (dd, J = 4.4, 2.9 Hz, 2H), 0.98 (dq, J = 7.0, 3.8 Hz, 2H). | m/z: 587.2 [M + H]⁺ |
| 173 | 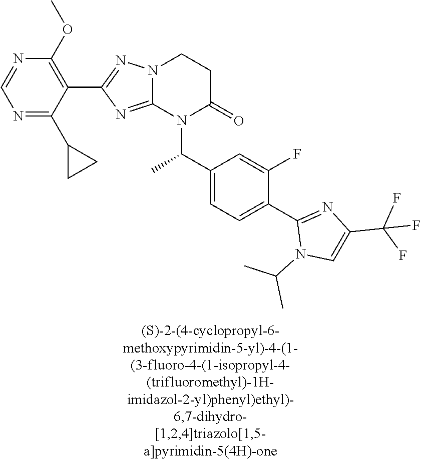 [SMILES: COc1ncnc(C2CC2)c1-c1nc2n(n1)CCC(=O)N2[C@@H](C)c1ccc(-c2nc(C(F)(F)F)cn2C(C)C)c(F)c1] | ¹H NMR (400 MHz, Chloroform-d) δ 8.60 (s, 1H), 7.46 (dd, J = 8.1, 7.2 Hz, 1H), 7.44-7.38 (m, 3H), 6.13 (d, J = 7.3 Hz, 1H), 4.39 (td, J = 7.3, 3.2 Hz, 2H), 4.29-4.21 (m, 1H), 3.95 (s, 3H), 3.11- 3.03 (m, 2H), 2.03 (s, 1H), 1.98 (d, J = 7.3 Hz, 3H), 1.40 (dd, J = 8.3, 6.7 Hz, 6H), 1.20 (dd, J = 3.6, 2.0 Hz, 2H), 0.95 (dd, J = 8.1, 1.7 Hz, 2H). | m/z: 585.2 [M + H]⁺ |
| 174 | 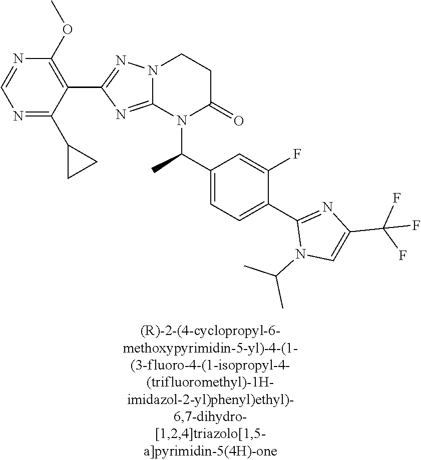 [SMILES: COc1ncnc(C2CC2)c1-c1nc2n(n1)CCC(=O)N2[C@H](C)c1ccc(-c2nc(C(F)(F)F)cn2C(C)C)c(F)c1] | ¹H NMR (400 MHz, Chloroform-d) δ 8.59 (s, 1H), 7.49-7.38 (m, 4H), 6.13 (d, J = 7.2 Hz, 1H), 4.39 (td, J = 7.3, 3.3 Hz, 2H), 4.28-4.20 (m, 1H), 3.94 (s, 3H), 3.07 (t, J = 7.2 Hz, 2H), 2.02 (d, J = 4.5 Hz, 1H), 1.97 (d, J = 7.3 Hz, 3H), 1.39 (dd, J = 8.3, 6.6 Hz, 6H), 1.19 (dt, J = 6.0, 4.1 Hz, 2H), 0.98-0.90 (m, 2H). | m/z: 585.2 [M + H]⁺ |
| 175 | 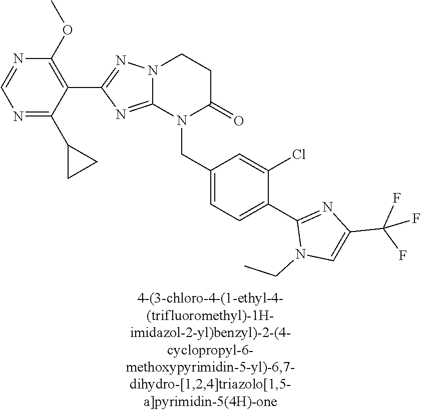 [SMILES: CCn1cc(C(F)(F)F)nc1-c1ccc(CN2C(=O)CCn3nc(-c4c(OC)ncnc4C4CC4)nc32)cc1Cl] | ¹H NMR (400 MHz, Chloroform-d) δ 8.62 (s, 1H), 7.68 (d, J = 1.5 Hz, 1H), 7.56 (dd, J = 7.9, 1.6 Hz, 1H), 7.40 (d, J = 7.9 Hz, 1H), 7.39-7.37 (m, 1H), 5.18 (s, 2H), 4.42 (t, J = 7.3 Hz, 2H), 3.97 (s, 3H), 3.83 (q, J = 7.3 Hz, 2H), 3.13 (t, J = 7.3 Hz, 2H), 2.05-2.01 (m, 1H), 1.34 (t, J = 7.3 Hz, 3H), 1.22 (dd, J = 4.5, 2.9 Hz, 2H), 0.98 (dq, J = 7.0, 3.8 Hz, 2H). | m/z: 573.3 [M + H]⁺ |
| 176 | 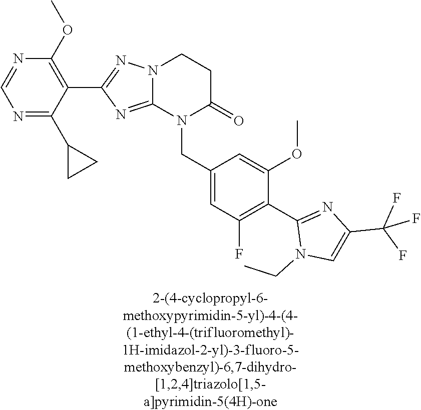 [SMILES: CCn1cc(C(F)(F)F)nc1-c1c(F)cc(CN2C(=O)CCn3nc(-c4c(OC)ncnc4C4CC4)nc32)cc1OC] | ¹H NMR (400 MHz, Chloroform-d) δ 8.62 (s, 1H), 7.39-7.37 (m, 1H), 7.07-7.02 (m, 1H), 6.99 (s, 1H), 5.15 (s, 2H), 4.41 (t, J = 7.3 Hz, 2H), 3.95 (s, 3H), 3.77 (d, J = 7.7 Hz, 5H), 3.12 (t, J = 7.3 Hz, 2H), 1.98 (tt, J = 8.2, 4.6 Hz, 1H), 1.33 (t, J = 7.3 Hz, 3H), 1.24-1.18 (m, 2H), 0.96 (dd, J = 8.0, 3.1 Hz, 2H). | m/z: 587.2 [M + H]⁺ |
| 177 | 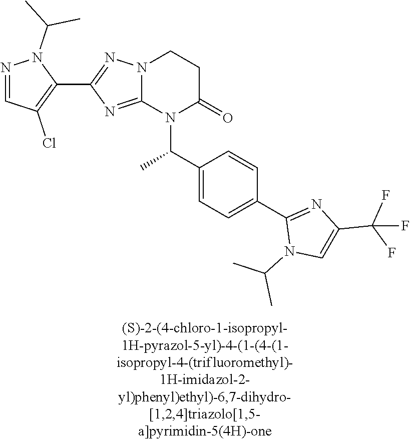 [SMILES: CC(C)n1cc(C(F)(F)F)nc1-c1ccc([C@H](C)N2C(=O)CCn3nc(-c4c(Cl)cnn4C(C)C)nc32)cc1] | ¹H NMR (400 MHz, Chloroform-d) δ 7.63 (d, J = 8.1 Hz, 2H), 7.55-7.44 (m, 3H), 7.39 (s, 1H), 6.24 (q, J = 7.1 Hz, 1H), 5.12 (m, 1H), 4.53 (m, 1H), 4.38 (m, 2H), 3.10 (t, J = 7.3 Hz, 2H), 1.99 (d, J = 7.2 Hz, 3H), 1.53-1.36 (m, 12H). | m/z: 561.2 [M + H]⁺ |
| 178 | 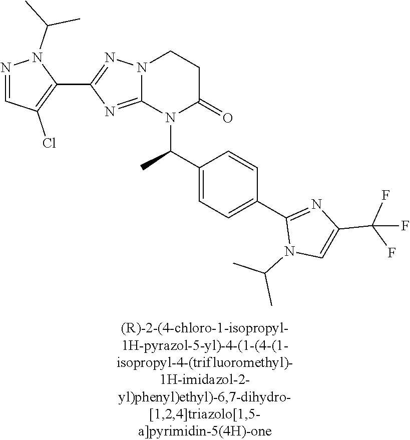 [SMILES: CC(C)n1cc(C(F)(F)F)nc1-c1ccc([C@@H](C)N2C(=O)CCn3nc(-c4c(Cl)cnn4C(C)C)nc32)cc1] | ¹H NMR (400 MHz, Chloroform-d) δ 7.63 (d, J = 8.2 Hz, 2H), 7.53-7.44 (m, 3H), 7.42-7.37 (m, 1H), 6.24 (q, J = 7.2 Hz, 1H), 5.12 (m, 1H), 4.53 (hept, J = 6.7 Hz, 1H), 4.46-4.31 (m, 2H), 3.09 (t, J = 7.3 Hz, 2H), 1.98 (d, J = 7.2 Hz, 3H), 1.51-1.37 (m, 12H). | m/z: 561.2 [M + H]⁺ |
| 179 | 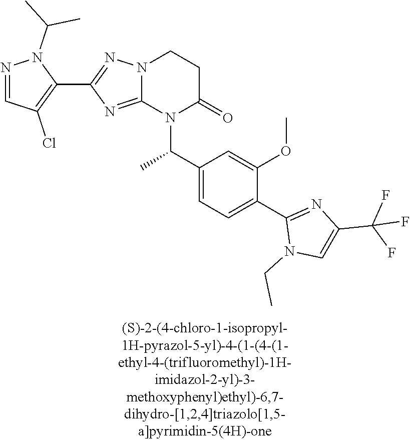 [SMILES: CCn1cc(C(F)(F)F)nc1-c1ccc([C@H](C)N2C(=O)CCn3nc(-c4c(Cl)cnn4C(C)C)nc32)cc1OC] | ¹H NMR (400 MHz, Chloroform-d) δ 7.51 (s, 1H), 7.38-7.32 (m, 2H), 7.25-7.17 (m, 2H), 6.20 (q, J = 7.2 Hz, 1H), 5.17 (m, 1H), 4.47-4.30 (m, 2H), 3.86-3.71 (m, 5H), 3.08 (t, J = 7.3 Hz, 2H), 1.99 (d, J = 7.3 Hz, 3H), 1.46 (dd, J = 8.3, 6.7 Hz, 6H), 1.31 (t, J = 7.3 Hz, 3H). | m/z: 577.2 [M + H]⁺ |
| 180 | 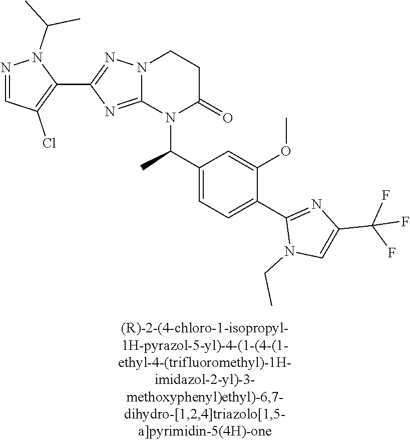 [SMILES: CCn1cc(C(F)(F)F)nc1-c1ccc([C@@H](C)N2C(=O)CCn3nc(-c4c(Cl)cnn4C(C)C)nc32)cc1OC] | ¹H NMR (400 MHz, Chloroform-d) δ 7.51 (s, 1H), 7.39-7.31 (m, 2H), 7.25-7.16 (m, 2H), 6.20 (q, J = 7.2 Hz, 1H), 5.17 (m, 1H), 4.46-4.30 (m, 2H), 3.87-3.69 (m, 5H), 3.09 (t, J = 7.3 Hz, 2H), 1.99 (d, J = 7.3 Hz, 3H), 1.46 (dd, J = 8.4, 6.6 Hz, 6H), 1.31 (t, J = 7.3 Hz, 3H). | m/z: 577.2 [M + H]⁺ |
| 181 | 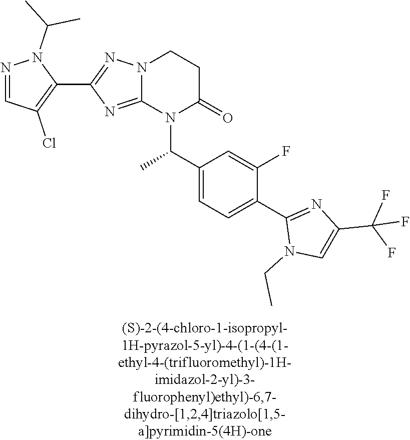 [SMILES: CCn1cc(C(F)(F)F)nc1-c1ccc([C@H](C)N2C(=O)CCn3nc(-c4c(Cl)cnn4C(C)C)nc32)cc1F] | ¹H NMR (400 MHz, Chloroform-d) δ 7.41-7.36 (m, 2H), 7.29 (d, J = 11.1 Hz, 2H), 7.13 (s, 1H), 6.10 (q, J = 7.1 Hz, 1H), 5.00 (m, 1H), 4.28 (h, J = 5.9 Hz, 2H), 3.77 (q, J = 7.2 Hz, 2H), 2.98 (t, J = 7.2 Hz, 2H), 1.85 (d, J = 7.2 Hz, 3H), 1.37 (d, J = 6.6 Hz, 3H), 1.31-1.23 (m, 6H). | m/z: 565.2 [M + H]⁺ |
| 182 | 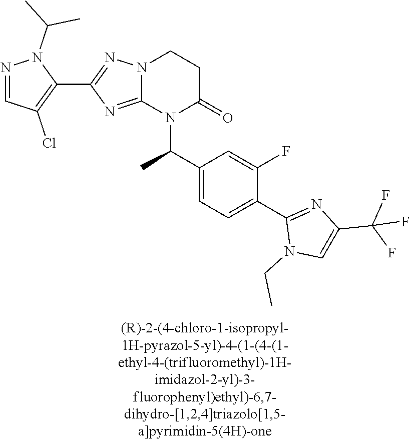 [SMILES: CCn1cc(C(F)(F)F)nc1-c1ccc([C@@H](C)N2C(=O)CCn3nc(-c4c(Cl)cnn4C(C)C)nc32)cc1F] | ¹H NMR (400 MHz, Chloroform-d) δ 7.39 (s, 2H), 7.29 (d, J = 11.6 Hz, 2H), 7.13 (s, 1H), 6.10 (d, J = 6.9 Hz, 1H), 5.06-4.95 (m, 1H), 4.28 (d, J = 6.3 Hz, 2H), 3.77 (d, J = 7.1 Hz, 2H), 2.98 (t, J = 7.0 Hz, 2H), 1.85 (d, J = 7.0 Hz, 3H), 1.37 (d, J = 6.2 Hz, 3H), 1.28 (dd, J = 16.9, 6.8 Hz, 6H). | m/z: 565.2 [M + H]⁺ |
| 183 | 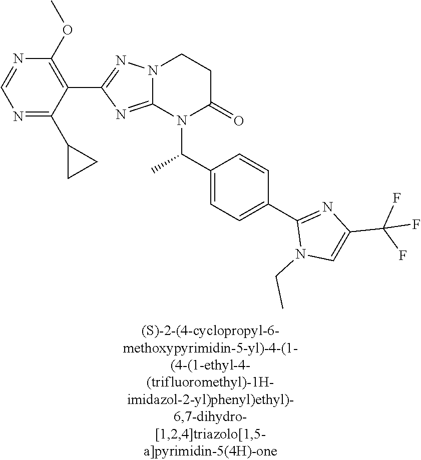 [SMILES: CCn1cc(C(F)(F)F)nc1-c1ccc([C@H](C)N2C(=O)CCn3nc(-c4c(OC)ncnc4C4CC4)nc32)cc1] | ¹H NMR (400 MHz, Chloroform-d) δ 8.60 (s, 1H), 7.63 (d, J = 8.2 Hz, 2H), 7.54- 7.48 (m, 2H), 7.35 (q, J = 1.2 Hz, 1H), 6.16 (q, J = 7.2 Hz, 1H), 4.39 (td, J = 7.3, 4.1 Hz, 2H), 4.06 (q, J = 7.3 Hz, 2H), 3.95 (s, 3H), 3.07 (t, J = 7.3 Hz, 2H), 1.99 (d, J = 7.4 Hz, 4H), 1.43 (t, J = 7.3 Hz, 3H), 1.19 (ddd, J = 9.2, 4.7, 3.1 Hz, 2H), 0.91 (dd, J = 8.2, 3.1 Hz, 2H). | m/z: 553.3 [M + H]⁺ |
| 184 | 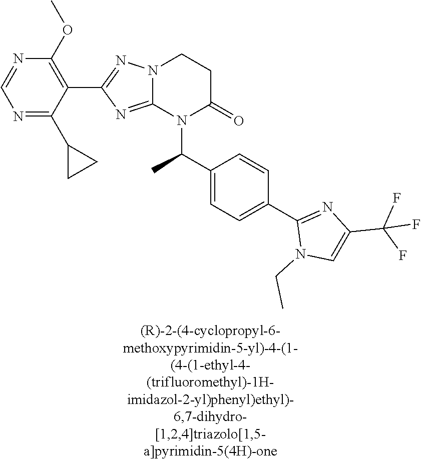 [SMILES: CCn1cc(C(F)(F)F)nc1-c1ccc([C@@H](C)N2C(=O)CCn3nc(-c4c(OC)ncnc4C4CC4)nc32)cc1] | ¹H NMR (400 MHz, Chloroform-d) δ 8.60 (s, 1H), 7.63 (d, J = 8.2 Hz, 2H), 7.53- 7.48 (m, 2H), 7.35 (d, J = 1.3 Hz, 1H), 6.16 (q, J = 7.2 Hz, 1H), 4.38 (td, J = 7.3, 4.2 Hz, 2H), 4.05 (q, J = 7.3 Hz, 2H), 3.95 (s, 3H), 3.07 (t, J = 7.3 Hz, 2H), 1.99 (d, J = 7.3 Hz, 4H), 1.43 (t, J = 7.3 Hz, 3H), 1.19 (ddd, J = 9.3, 4.7, 3.1 Hz, 2H), 0.91 (dd, J = 8.2, 3.1 Hz, 2H). | m/z: 553.3 [M + H]⁺ |
| 185 | 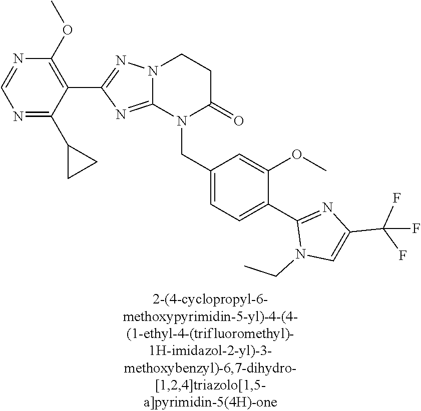 [SMILES: CCn1cc(C(F)(F)F)nc1-c1ccc(CN2C(=O)CCn3nc(-c4c(OC)ncnc4C4CC4)nc32)cc1OC] | ¹H NMR (400 MHz, Chloroform-d) δ 8.64 (s, 1H), 7.36 (d, J = 7.6 Hz, 2H), 7.25- 7.16 (m, 2H), 5.19 (s, 2H), 4.40 (t, J = 7.3 Hz, 2H), 3.96 (s, 3H), 3.85-3.75 (m, 5H), 3.12 (t, J = 7.3 Hz, 2H), 1.97 (td, J = 8.8, 8.0, 4.9 Hz, 1H), 1.34 (t, J = 7.3 Hz, 3H), 1.23 (dd, J = 7.6, 3.3 Hz, 2H), 0.96 (dq, J = 6.9, 3.8 Hz, 2H). | m/z: 569.2 [M + H]⁺ |
| 186 | 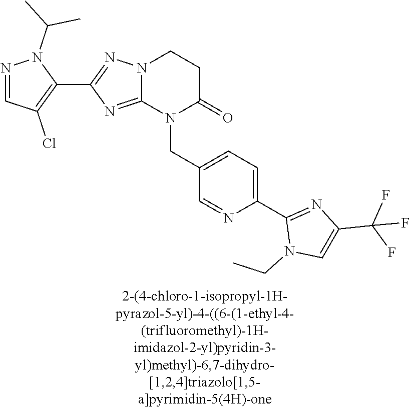 [SMILES: CCn1cc(C(F)(F)F)nc1-c1ccc(CN2C(=O)CCn3nc(-c4c(Cl)cnn4C(C)C)nc32)cn1] | ¹H NMR (400 MHz, Chloroform-d) δ 8.81 (d, J = 2.1 Hz, 1H), 8.18 (d, J = 8.2 Hz, 1H), 7.98 (dd, J = 8.2, 2.2 Hz, 1H), 7.55 (s, 1H), 7.33 (s, 1H), 5.26-5.17 (m, 3H), 4.63 (q, J = 7.2 Hz, 2H), 4.41 (t, J = 7.3 Hz, 2H), 3.13 (t, J = 7.3 Hz, 2H), 1.50 (d, J = 6.6 Hz, 6H), 1.45 (t, J = 7.2 Hz, 3H). | m/z: 534.1 [M + H]⁺ |
| 187 | 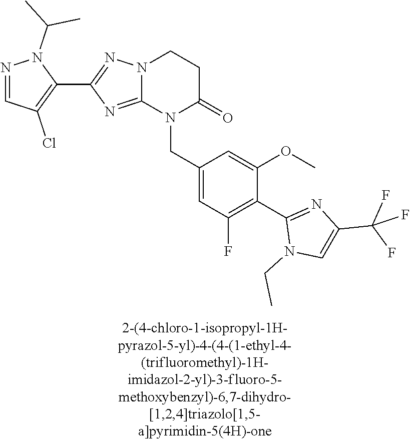 [SMILES: CCn1cc(C(F)(F)F)nc1-c1c(F)cc(CN2C(=O)CCn3nc(-c4c(Cl)cnn4C(C)C)nc32)cc1OC] | ¹H NMR (400 MHz, Chloroform-d) δ 7.54 (s, 1H), 7.39 (s, 1H), 7.04 (d, J = 9.2 Hz, 1H), 6.99 (s, 1H), 5.26- 5.12 (m, 3H), 4.42 (t, J = 7.3 Hz, 2H), 3.78 (d, J = 5.9 Hz, 5H), 3.13 (t, J = 7.3 Hz, 2H), 1.50 (d, J = 6.6 Hz, 6H), 1.34 (t, J = 7.3 Hz, 3H). | m/z: 581.1 [M + H]⁺ |
| 188 | 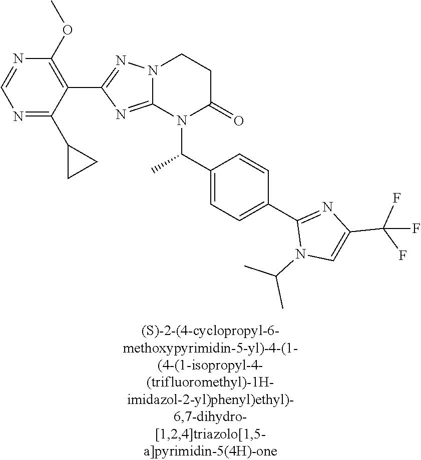 [SMILES: COc1ncnc(C2CC2)c1-c1nc2n(n1)CCC(=O)N2[C@@H](C)c1ccc(-c2nc(C(F)(F)F)cn2C(C)C)cc1] | ¹H NMR (400 MHz, DMSO- d₆) δ 8.61 (s, 1H), 8.16 (d, J = 1.3 Hz, 1H), 7.57-7.45 (m, 4H), 6.07 (q, J = 7.0 Hz, 1H), 4.44 (dt, J = 10.0, 7.0 Hz, 3H), 3.84 (s, 3H), 3.15 (t, J = 7.2 Hz, 2H), 1.87 (d, J = 7.2 Hz, 4H), 1.40 (t, J = 7.0 Hz, 6H), 0.97 (dq, J = 9.1, 4.7 Hz, 2H), 0.86-0.78 (m, 2H). | m/z: 567.2 [M + H]⁺ |
| 189 | 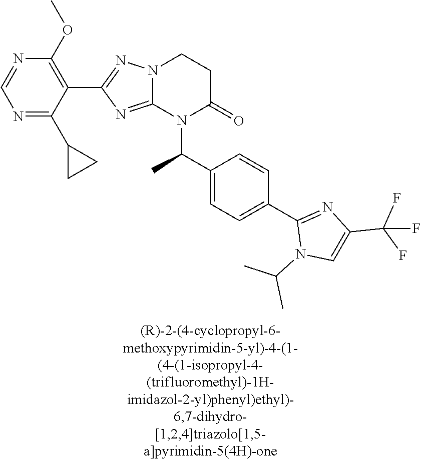 [SMILES: COc1ncnc(C2CC2)c1-c1nc2n(n1)CCC(=O)N2[C@H](C)c1ccc(-c2nc(C(F)(F)F)cn2C(C)C)cc1] | ¹H NMR (400 MHz, DMSO- d₆) δ 8.61 (s, 1H), 8.17 (d, J = 1.4 Hz, 1H), 7.57-7.45 (m, 4H), 6.07 (q, J = 7.0 Hz, 1H), 4.44 (dt, J = 10.0, 6.9 Hz, 3H), 3.84 (s, 3H), 3.15 (t, J = 7.2 Hz, 2H), 1.87 (d, J = 7.2 Hz, 4H), 1.40 (t, J = 7.0 Hz, 6H), 0.97 (dq, J = 8.9, 4.6 Hz, 2H), 0.83 (dtd, J = 11.8, 6.5, 5.9, 3.5 Hz, 2H). | m/z: 567.2 [M + H]⁺ |
| 190 | 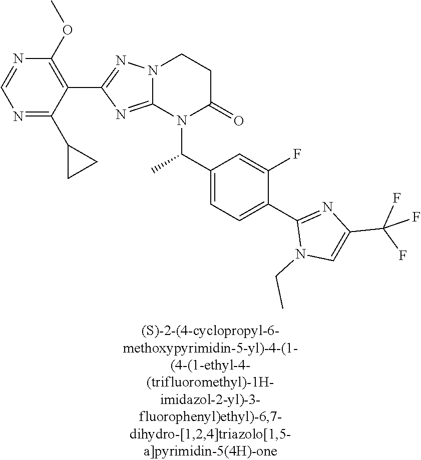 [SMILES: CCn1cc(C(F)(F)F)nc1-c1ccc([C@H](C)N2C(=O)CCn3nc(-c4c(OC)ncnc4C4CC4)nc32)cc1F] | ¹H NMR (400 MHz, Chloroform-d) δ 8.60 (s, 1H), 7.49 (t, J = 7.8 Hz, 1H), 7.46- 7.36 (m, 3H), 6.14 (q, J = 7.2 Hz, 1H), 4.39 (td, J = 7.3, 3.5 Hz, 2H), 3.93 (d, J = 22.5 Hz, 5H), 3.08 (t, J = 7.3 Hz, 2H), 2.07-1.95 (m, 4H), 1.38 (t, J = 7.3 Hz, 3H), 1.22-1.15 (m, 2H), 0.95 (dd, J = 8.1, 1.9 Hz, 2H). | m/z: 571.2 [M + H]⁺ |
| 191 | 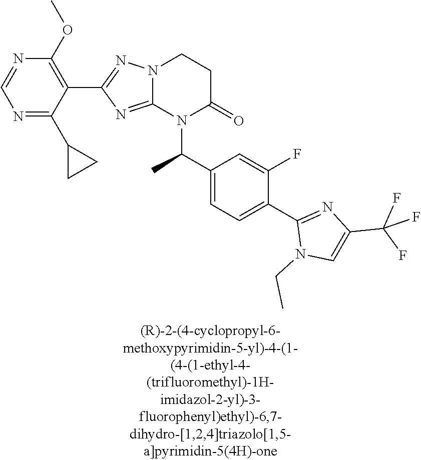 [SMILES: CCn1cc(C(F)(F)F)nc1-c1ccc([C@@H](C)N2C(=O)CCn3nc(-c4c(OC)ncnc4C4CC4)nc32)cc1F] | ¹H NMR (400 MHz, Chloroform-d) δ 8.60 (s, 1H), 7.48 (t, J = 7.8 Hz, 1H), 7.44- 7.38 (m, 3H), 6.13 (q, J = 7.2 Hz, 1H), 4.39 (td, J = 7.3, 3.5 Hz, 2H), 3.95 (s, 3H), 3.90 (q, J = 7.3 Hz, 2H), 3.07 (t, J = 7.3 Hz, 2H), 2.02 (s, 1H), 1.97 (d, J = 7.2 Hz, 3H), 1.38 (t, J = 7.3 Hz, 3H), 1.18 (d, J = 4.9 Hz, 2H), 0.94 (dd, J = 8.1, 4.9 Hz, 2H). | m/z: 571.2 [M + H]⁺ |
| 192 | 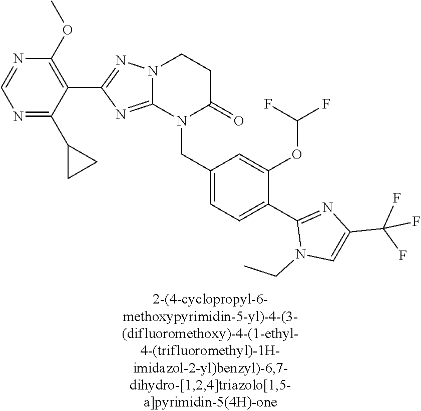 [SMILES: CCn1cc(C(F)(F)F)nc1-c1ccc(CN2C(=O)CCn3nc(-c4c(OC)ncnc4C4CC4)nc32)cc1OC(F)F] | ¹H NMR (400 MHz, Chloroform-d) δ 8.63 (s, 1H), 7.56-7.34 (m, 4H), 6.39 (t, J = 73.8 Hz, 1H), 5.20 (s, 2H), 4.42 (t, J = 7.3 Hz, 2H), 3.96 (s, 3H), 3.86 (q, J = 7.3 Hz, 2H), 3.13 (t, J = 7.3 Hz, 2H), 2.04-1.95 (m, 1H), 1.37 (t, J = 7.3 Hz, 3H), 1.24-1.20 (m, 2H), 0.96 (dd, J = 7.9, 2.9 Hz, 2H). | m/z: 605.1 [M + H]⁺ |
| 193 | 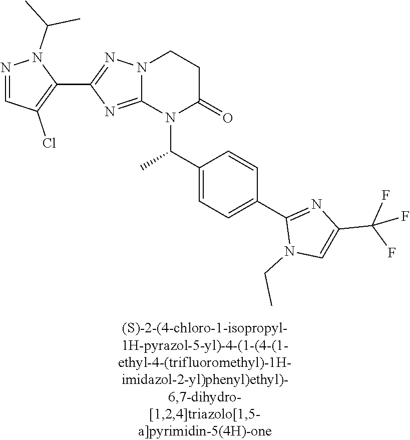 [SMILES: CCn1cc(C(F)(F)F)nc1-c1ccc([C@H](C)N2C(=O)CCn3nc(-c4c(Cl)cnn4C(C)C)nc32)cc1] | ¹H NMR (400 MHz, Chloroform-d) δ 7.63 (d, J = 8.2 Hz, 2H), 7.55-7.49 (m, 3H), 7.34 (s, 1H), 6.24 (q, J = 7.2 Hz, 1H), 5.12 (m, 1H), 4.47-4.32 (m, 2H), 4.04 (q, J = 7.3 Hz, 2H), 3.10 (t, J = 7.3 Hz, 2H), 1.99 (d, J = 7.2 Hz, 3H), 1.48 (d, J = 6.6 Hz, 3H), 1.42 (t, J = 6.9 Hz, 6H). | m/z: 547.2 [M + H]⁺ |
| 194 | 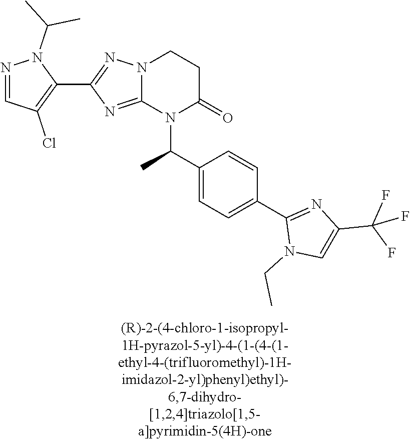 [SMILES: CCn1cc(C(F)(F)F)nc1-c1ccc([C@@H](C)N2C(=O)CCn3nc(-c4c(Cl)cnn4C(C)C)nc32)cc1] | ¹H NMR (400 MHz, Chloroform-d) δ 7.63 (d, J = 8.2 Hz, 2H), 7.55-7.49 (m, 3H), 7.34 (s, 1H), 6.24 (q, J = 7.2 Hz, 1H), 5.12 (m, 1H), 4.47-4.32 (m, 2H), 4.04 (q, J = 7.3 Hz, 2H), 3.10 (t, J = 7.3 Hz, 2H), 1.99 (d, J = 7.2 Hz, 3H), 1.48 (d, J = 6.6 Hz, 3H), 1.42 (t, J = 6.9 Hz, 6H). | m/z: 547.2 [M + H]⁺ |
| 195 | 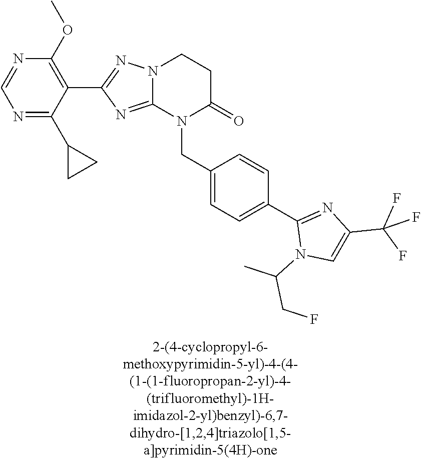 [SMILES: COc1ncnc(C2CC2)c1-c1nc2n(n1)CCC(=O)N2Cc1ccc(-c2nc(C(F)(F)F)cn2C(C)CF)cc1] | ¹H NMR (400 MHz, Chloroform-d) δ 8.61 (s, 1H), 7.64 (d, J = 8.0 Hz, 2H), 7.49- 7.44 (m, 3H), 5.20 (s, 2H), 4.63 (dtd, J = 20.0, 6.6, 4.2 Hz, 1H), 4.58-4.53 (m, 1H), 4.47-4.42 (m, 1H), 4.39 (t, J = 7.3 Hz, 2H), 3.96 (s, 3H), 3.11 (t, J = 7.3 Hz, 2H), 2.03 (tt, J = 8.4, 4.6 Hz, 1H), 1.48 (dd, J = 7.0, 1.2 Hz, 3H), 1.21 (dt, J = 6.4, 3.3 Hz, 2H), 0.94 (dq, J = 7.1, 3.8 Hz, 2H). | m/z: 571.4 [M + H]⁺ |
| 196 | 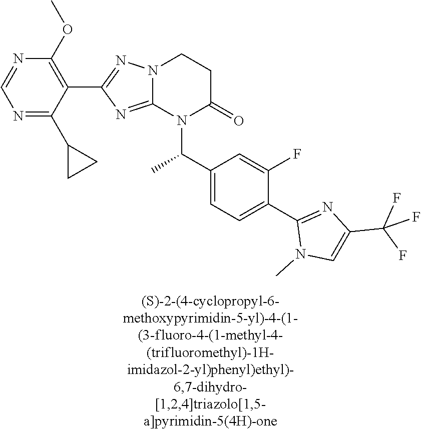 [SMILES: COc1ncnc(C2CC2)c1-c1nc2n(n1)CCC(=O)N2[C@@H](C)c1ccc(-c2nc(C(F)(F)F)cn2C)c(F)c1] | ¹H NMR (400 MHz, Chloroform-d) δ 8.61 (s, 1H), 7.54 (t, J = 7.7 Hz, 1H), 7.42 (t, J = 8.8 Hz, 2H), 7.34 (s, 1H), 6.14 (d, J = 7.3 Hz, 1H), 4.40 (q, J = 3.9 Hz, 2H), 3.96 (s, 3H), 3.63 (d, J = 2.2 Hz, 3H), 3.08 (t, J = 7.3 Hz, 2H), 1.98 (d, J = 7.2 Hz, 4H), 1.20 (d, J = 4.1 Hz, 2H), 0.94 (dd, J = 8.1, 2.3 Hz, 2H). | m/z: 557.2 [M + H]⁺ |
| 197 | 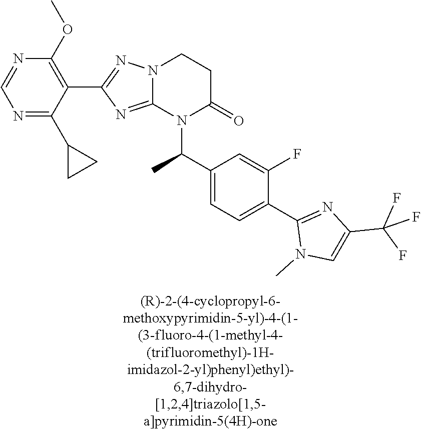 [SMILES: COc1ncnc(C2CC2)c1-c1nc2n(n1)CCC(=O)N2[C@H](C)c1ccc(-c2nc(C(F)(F)F)cn2C)c(F)c1] | ¹H NMR (400 MHz, Chloroform-d) δ 8.61 (s, 1H), 7.54 (t, J = 7.7 Hz, 1H), 7.42 (t, J = 8.8 Hz, 2H), 7.34 (s, 1H), 6.14 (d, J = 7.3 Hz, 1H), 4.40 (q, J = 3.9 Hz, 2H), 3.96 (s, 3H), 3.63 (d, J = 2.2 Hz, 3H), 3.08 (t, J = 7.3 Hz, 2H), 1.98 (d, J = 7.2 Hz, 4H), 1.20 (d, J = 4.1 Hz, 2H), 0.94 (dd, J = 8.1, 2.3 Hz, 2H). | m/z: 557.2 [M + H]⁺ |
| 198 | 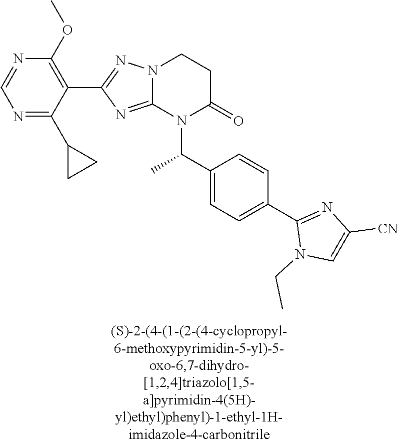 [SMILES: CCn1cc(C#N)nc1-c1ccc([C@H](C)N2C(=O)CCn3nc(-c4c(OC)ncnc4C4CC4)nc32)cc1] | ¹H NMR (400 MHz, Chloroform-d) δ 8.56 (s, 1H), 7.61 (d, J = 7.8 Hz, 2H), 7.55 (s, 1H), 7.46 (d, J = 7.8 Hz, 2H), 6.14 (q, J = 7.1 Hz, 1H), 4.37 (dt, J = 7.0, 3.5 Hz, 2H), 4.04 (q, J = 7.2 Hz, 2H), 3.92 (s, 3H), 3.06 (t, J = 7.3 Hz, 2H), 1.96 (d, J = 7.0 Hz, 4H), 1.39 (t, J = 7.3 Hz, 3H), 1.18- 1.10 (m, 2H), 0.87 (d, J = 9.1 Hz, 2H). | m/z: 510.2 [M + H]⁺ |
| 199 | 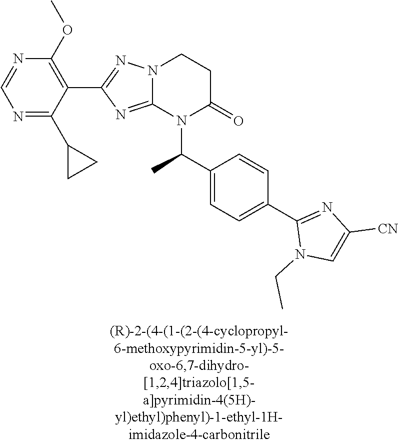 [SMILES: CCn1cc(C#N)nc1-c1ccc([C@@H](C)N2C(=O)CCn3nc(-c4c(OC)ncnc4C4CC4)nc32)cc1] | ¹H NMR (400 MHz, Chloroform-d) δ 8.56 (s, 1H), 7.61 (d, J = 7.8 Hz, 2H), 7.55 (s, 1H), 7.46 (d, J = 7.8 Hz, 2H), 6.14 (q, J = 7.1 Hz, 1H), 4.37 (dt, J = 7.0, 3.5 Hz, 2H), 4.04 (q, J = 7.2 Hz, 2H), 3.92 (s, 3H), 3.06 (t, J = 7.3 Hz, 2H), 1.96 (d, J = 7.0 Hz, 4H), 1.39 (t, J = 7.3 Hz, 3H), 1.18- 1.10 (m, 2H), 0.87 (d, J = 9.1 Hz, 2H). | m/z: 510.2 [M + H]⁺ |
| 200 | 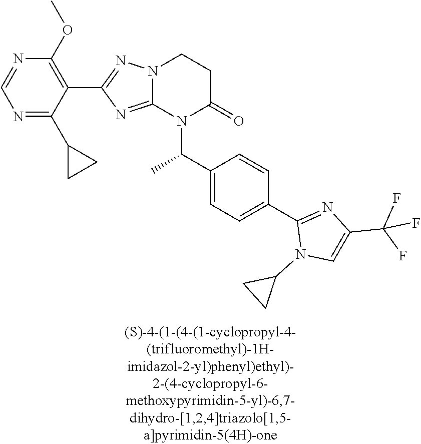 [SMILES: COc1ncnc(C2CC2)c1-c1nc2n(n1)CCC(=O)N2[C@@H](C)c1ccc(-c2nc(C(F)(F)F)cn2C2CC2)cc1] | ¹H NMR (400 MHz, Chloroform-d) δ 8.59 (d, J = 1.2 Hz, 1H), 7.77 (d, J = 7.9 Hz, 2H), 7.59 (d, J = 8.0 Hz, 2H), 7.30 (s, 1H), 6.16 (q, J = 7.2 Hz, 1H), 4.38 (q, J = 6.9 Hz, 2H), 3.97-3.93 (m, 3H), 3.47 (tt, J = 7.5, 4.0 Hz, 1H), 3.07 (t, J = 7.3 Hz, 2H), 1.98 (d, J = 7.0 Hz, 4H), 1.17 (d, J = 10.3 Hz, 2H), 1.05 (d, J = 7.4 Hz, 2H), 0.89 (dt, J = 7.3, 3.8 Hz, 4H). | m/z: 565.2 [M + H]⁺ |
| 201 | 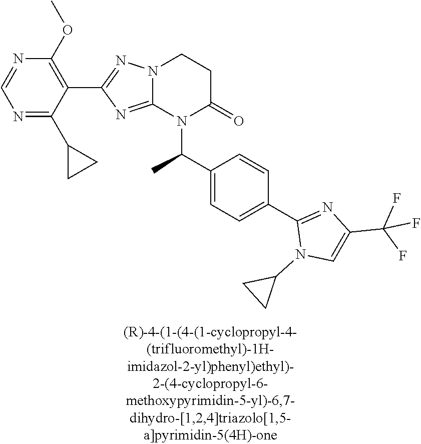 [SMILES: COc1ncnc(C2CC2)c1-c1nc2n(n1)CCC(=O)N2[C@H](C)c1ccc(-c2nc(C(F)(F)F)cn2C2CC2)cc1] | ¹H NMR (400 MHz, Chloroform-d) δ 8.59 (d, J = 1.2 Hz, 1H), 7.77 (d, J = 7.9 Hz, 2H), 7.59 (d, J = 8.0 Hz, 2H), 7.30 (s, 1H), 6.16 (q, J = 7.2 Hz, 1H), 4.38 (q, J = 6.9 Hz, 2H), 3.97-3.93 (m, 3H), 3.47 (tt, J = 7.5, 4.0 Hz, 1H), 3.07 (t, J = 7.3 Hz, 2H), 1.98 (d, J = 7.0 Hz, 4H), 1.17 (d, J = 10.3 Hz, 2H), 1.05 (d, J = 7.4 Hz, 2H), 0.89 (dt, J = 7.3, 3.8 Hz, 4H). | m/z: 565.2 [M + H]⁺ |
| 202 | 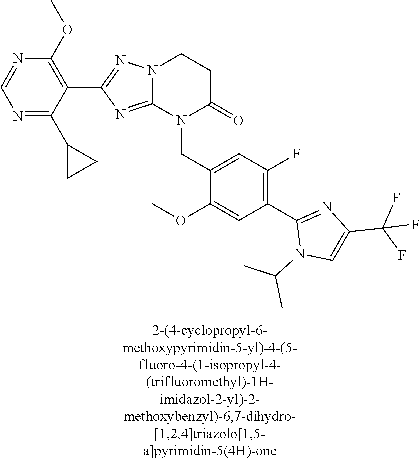 [SMILES: COc1cc(-c2nc(C(F)(F)F)cn2C(C)C)c(F)cc1CN1C(=O)CCn2nc(-c3c(OC)ncnc3C3CC3)nc21] | ¹H NMR (400 MHz, Chloroform-d) δ 8.60 (s, 1H), 7.42 (d, J = 1.2 Hz, 1H), 7.02- 6.96 (m, 2H), 5.22 (s, 2H), 4.48 (t, J = 7.3 Hz, 2H), 4.26 (td, J = 6.7, 1.3 Hz, 1H), 3.93 (s, 3H), 3.84 (s, 3H), 3.17 (t, J = 7.3 Hz, 2H), 2.03-1.94 (m, 1H), 1.40 (d, J = 6.6 Hz, 6H), 1.19 (dd, J = 4.5, 2.8 Hz, 2H), 0.93 (dd, J = 8.1, 3.0 Hz, 2H). | m/z: 601.1 [M + H]⁺ |
| 203 | 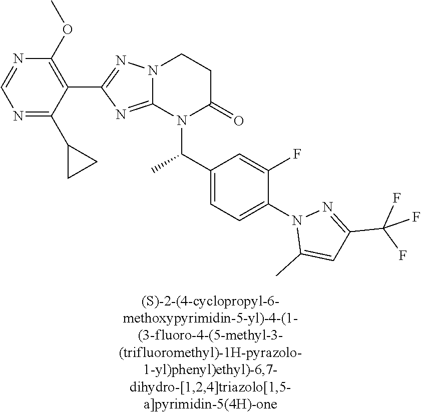 [SMILES: COc1ncnc(C2CC2)c1-c1nc2n(n1)CCC(=O)N2[C@@H](C)c1ccc(-n2nc(C(F)(F)F)cc2C)c(F)c1] | ¹H NMR (400 MHz, Chloroform-d) δ 8.61 (s, 1H), 7.53 (dd, J = 11.1, 1.3 Hz, 1H), 7.49-7.43 (m, 1H), 7.39 (t, J = 7.8 Hz, 1H), 6.44 (s, 1H), 6.15 (q, J = 7.2 Hz, 1H), 4.40 (td, J = 7.3, 3.2 Hz, 2H), 3.96 (s, 3H), 3.09 (t, J = 7.3 Hz, 2H), 2.23 (s, 3H), 2.00 (dd, J = 13.1, 5.8 Hz, 4H), 1.24-1.18 (m, 2H), 0.96 (dd, J = 8.0, 2.0 Hz, 2H). | m/z: 557.1 [M + H]⁺ |
| 204 | 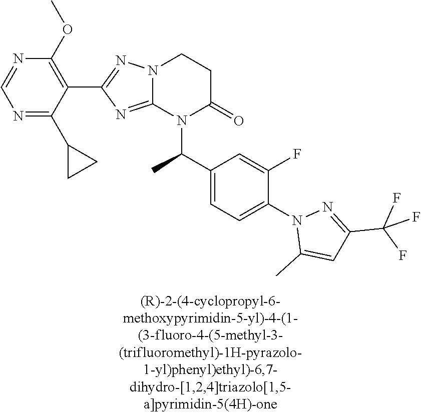 [SMILES: COc1ncnc(C2CC2)c1-c1nc2n(n1)CCC(=O)N2[C@H](C)c1ccc(-n2nc(C(F)(F)F)cc2C)c(F)c1] | ¹H NMR (400 MHz, Chloroform-d) δ 8.61 (s, 1H), 7.53 (dd, J = 11.1, 1.3 Hz, 1H), 7.49-7.43 (m, 1H), 7.39 (t, J = 7.8 Hz, 1H), 6.44 (s, 1H), 6.15 (q, J = 7.2 Hz, 1H), 4.40 (td, J = 7.3, 3.2 Hz, 2H), 3.96 (s, 3H), 3.09 (t, J = 7.3 Hz, 2H), 2.23 (s, 3H), 2.00 (dd, J = 13.1, 5.8 Hz, 4H), 1.24-1.18 (m, 2H), 0.96 (dd, J = 8.0, 2.0 Hz, 2H). | m/z: 557.1 [M + H]⁺ |
| 205 | 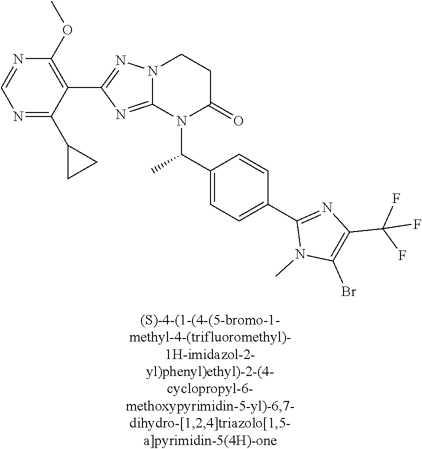 [SMILES: COc1ncnc(C2CC2)c1-c1nc2n(n1)CCC(=O)N2[C@@H](C)c1ccc(-c2nc(C(F)(F)F)c(Br)n2C)cc1] | ¹H NMR (400 MHz, Chloroform-d) δ 8.60 (s, 1H), 7.64 (d, J = 8.2 Hz, 2H), 7.51 (d, J = 8.3 Hz, 2H), 6.16 (q, J = 7.2 Hz, 1H), 4.39 (td, J = 7.3, 4.2 Hz, 2H), 3.95 (s, 3H), 3.68 (s, 3H), 3.07 (t, J = 7.3 Hz, 2H), 1.98 (d, J = 7.2 Hz, 4H), 1.19 (dd, J = 12.1, 4.4 Hz, 2H), 0.91 (dd, J = 8.1, 3.0 Hz, 2H). | m/z: 617.0 [M + H]⁺ |
| 206 | 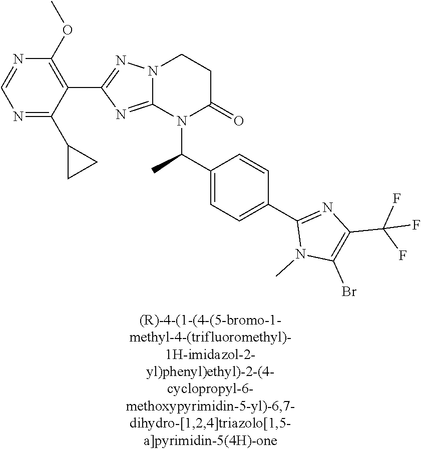 [SMILES: COc1ncnc(C2CC2)c1-c1nc2n(n1)CCC(=O)N2[C@H](C)c1ccc(-c2nc(C(F)(F)F)c(Br)n2C)cc1] | ¹H NMR (400 MHz, Chloroform-d) δ 8.60 (s, 1H), 7.64 (d, J = 8.2 Hz, 2H), 7.51 (d, J = 8.3 Hz, 2H), 6.16 (q, J = 7.2 Hz, 1H), 4.39 (td, J = 7.3, 4.2 Hz, 2H), 3.95 (s, 3H), 3.68 (s, 3H), 3.07 (t, J = 7.3 Hz, 2H), 1.98 (d, J = 7.2 Hz, 4H), 1.19 (dd, J = 12.1, 4.4 Hz, 2H), 0.91 (dd, J = 8.1, 3.0 Hz, 2H). | m/z: 617.0 [M + H]⁺ |
| 207 | 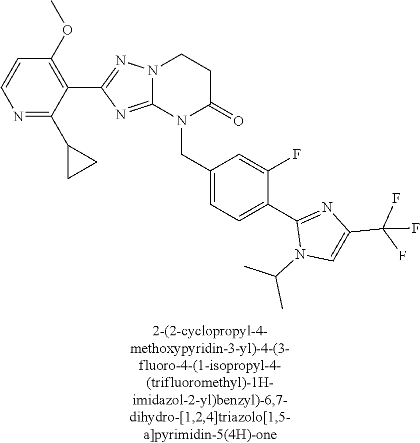 [SMILES: COc1ccnc(C2CC2)c1-c1nc2n(n1)CCC(=O)N2Cc1ccc(-c2nc(C(F)(F)F)cn2C(C)C)c(F)c1] | ¹H NMR (400 MHz, Chloroform-d) δ 8.40 (d, J = 5.8 Hz, 1H), 7.44 (dd, J = 13.3, 1.4 Hz, 4H), 6.69 (d, J = 5.8 Hz, 1H), 5.21 (s, 2H), 4.41 (s, 2H), 4.23 (td, J = 6.7, 1.2 Hz, 1H), 3.83 (s, 3H), 3.13 (t, J = 7.3 Hz, 2H), 1.89 (d, J = 4.8 Hz, 1H), 1.40 (d, J = 6.7 Hz, 6H), 1.14 (s, 2H), 0.88- 0.84 (m, 2H). | m/z: 570.2 [M + H]⁺ |
| 208 | 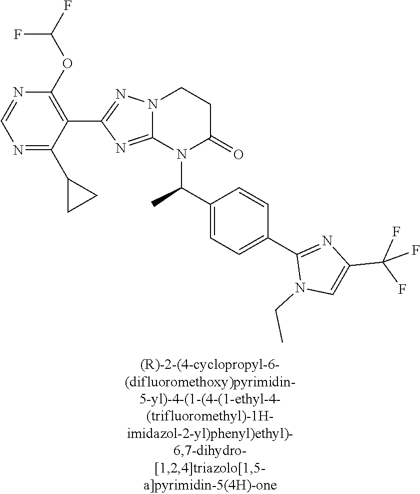 [SMILES: CCn1cc(C(F)(F)F)nc1-c1ccc([C@@H](C)N2C(=O)CCn3nc(-c4c(OC(F)F)ncnc4C4CC4)nc32)cc1] | ¹H NMR (400 MHz, Chloroform-d) δ 8.62 (s, 1H), 7.65 (d, J = 8.2 Hz, 2H), 7.52 (s, 1H), 7.52 (t, J = 72.0 Hz, 1H), 7.50 (s, 1H), 7.36-7.34 (m, 1H), 6.18 (q, J = 7.2 Hz, 1H), 4.39 (td, J = 7.2, 4.1 Hz, 2H), 4.05 (q, J = 7.3 Hz, 2H), 3.09 (t, J = 7.3 Hz, 2H), 2.19 (dq, J = 8.4, 4.7, 4.2 Hz, 1H), 1.99 (d, J = 7.3 Hz, 3H), 1.43 (t, J = 7.3 Hz, 3H), 1.25-1.18 (m, 2H), 1.01 (dd, J = 7.7, 4.4 Hz, 2H). | m/z: 589.2 [M + H]⁺ |
| 209 | 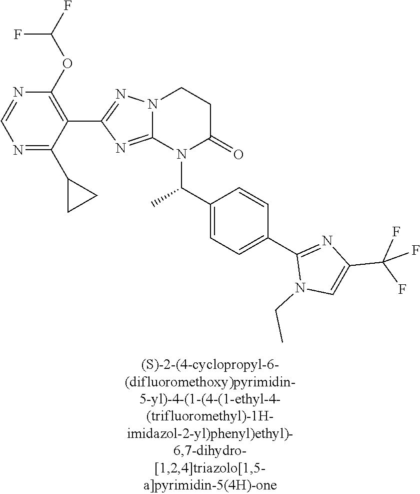 [SMILES: CCn1cc(C(F)(F)F)nc1-c1ccc([C@H](C)N2C(=O)CCn3nc(-c4c(OC(F)F)ncnc4C4CC4)nc32)cc1] | ¹H NMR (400 MHz, Chloroform-d) δ 8.62 (s, 1H), 7.65 (d, J = 8.2 Hz, 2H), 7.52 (s, 1H), 7.52 (t, J = 72.0 Hz, 1H), 7.50 (s, 1H), 7.36-7.34 (m, 1H), 6.18 (q, J = 7.2 Hz, 1H), 4.39 (td, J = 7.2, 4.1 Hz, 2H), 4.05 (q, J = 7.3 Hz, 2H), 3.09 (t, J = 7.3 Hz, 2H), 2.19 (tt, J = 8.4, 4.6 Hz, 1H), 1.99 (d, J = 7.3 Hz, 3H), 1.43 (t, J = 7.3 Hz, 3H), 1.25-1.18 (m, 2H), 1.01 (dd, J = 7.7, 4.4 Hz, 2H). | m/z: 589.2 [M + H]⁺ |
| 210 | 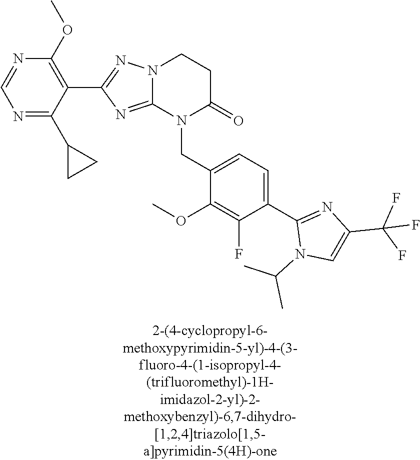 [SMILES: COc1ncnc(C2CC2)c1-c1nc2n(n1)CCC(=O)N2Cc1ccc(-c2nc(C(F)(F)F)cn2C(C)C)c(F)c1OC] | ¹H NMR (400 MHz, Chloroform-d) δ 8.61 (s, 1H), 7.43 (q, J = 1.2 Hz, 1H), 7.18- 7.09 (m, 2H), 5.27 (s, 2H), 4.45 (t, J = 7.2 Hz, 2H), 4.24 (m, 1H), 3.98 (d, J = 2.0 Hz, 3H), 3.94 (s, 3H), 3.15 (t, J = 7.2 Hz, 2H), 1.99 (tt, J = 8.2, 4.5 Hz, 1H), 1.41 (d, J = 6.6 Hz, 6H), 1.22-1.17 (m, 2H), 0.93 (dq, J = 7.1, 3.9 Hz, 2H). | m/z: 601.1 [M + H]⁺ |
| 211 | 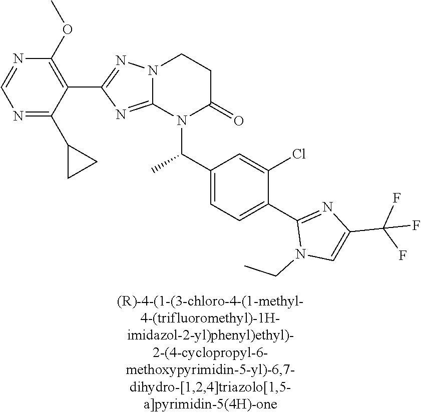 [SMILES: CCn1cc(C(F)(F)F)nc1-c1ccc([C@H](C)N2C(=O)CCn3nc(-c4c(OC)ncnc4C4CC4)nc32)cc1Cl] | ¹H NMR (400 MHz, Chloroform-d) δ 8.61 (s, 1H), 7.66 (d, J = 1.3 Hz, 1H), 7.55 (dd, J = 8.0, 1.4 Hz, 1H), 7.42-7.36 (m, 2H), 6.11 (q, J = 7.2 Hz, 1H), 4.40 (t, J = 7.0 Hz, 2H), 3.96 (s, 3H), 3.84 (q, J = 7.3 Hz, 2H), 3.08 (td, J = 7.2, 1.7 Hz, 2H), 2.04 (dt, J = 8.0, 3.5 Hz, 1H), 1.98 (d, J = 7.3 Hz, 3H), 1.35 (s, 3H), 1.21 (dt, J = 7.8, 3.5 Hz, 2H), 0.97 (dd, J = 7.9, 3.4 Hz, 2H). | m/z: 587.2 [M + H]⁺ |
| 212 | 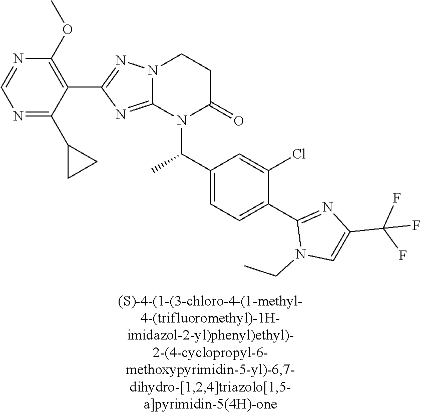 [SMILES: CCn1cc(C(F)(F)F)nc1-c1ccc([C@H](C)N2C(=O)CCn3nc(-c4c(OC)ncnc4C4CC4)nc32)cc1Cl] | ¹H NMR (400 MHz, Chloroform-d) δ 8.61 (s, 1H), 7.66 (s, 1H), 7.55 (d, J = 7.9 Hz, 1H), 7.42-7.36 (m, 2H), 6.11 (q, J = 7.2 Hz, 1H), 4.40 (t, J = 7.1 Hz, 2H), 3.96 (s, 3H), 3.84 (q, J = 7.3 Hz, 2H), 3.11-3.05 (m, 2H), 2.04 (tt, J = 8.2, 4.7 Hz, 1H), 1.98 (d, J = 7.2 Hz, 3H), 1.35 (t, J = 7.4 Hz, 3H), 1.23-1.19 (m, 2H), 0.97 (dd, J = 7.9, 3.4 Hz, 2H). | m/z: 587.2 [M + H]⁺ |
| 213 | 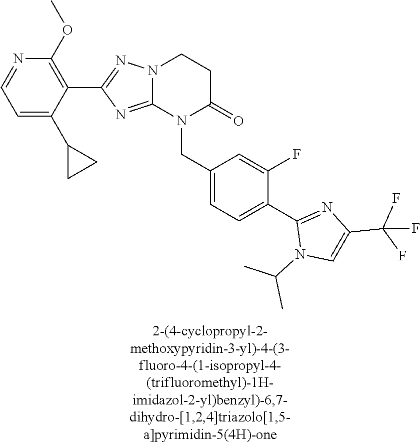 [SMILES: COc1nccc(C2CC2)c1-c1nc2n(n1)CCC(=O)N2Cc1ccc(-c2nc(C(F)(F)F)cn2C(C)C)c(F)c1] | ¹H NMR (400 MHz, Chloroform-d) δ 8.08 (d, J = 5.5 Hz, 1H), 7.51-7.39 (m, 4H), 6.36 (d, J = 5.6 Hz, 1H), 5.20 (s, 2H), 4.41 (t, J = 7.3 Hz, 2H), 4.27-4.19 (m, 1H), 3.92 (s, 3H), 3.12 (t, J = 7.3 Hz, 2H), 1.84 (dd, J = 9.2, 4.2 Hz, 1H), 1.40 (d, J = 6.7 Hz, 6H), 0.96 (dd, J = 8.3, 2.1 Hz, 2H), 0.79 (dd, J = 4.8, 1.8 Hz, 2H). | m/z: 570.3 [M + H]⁺ |
| 214 | 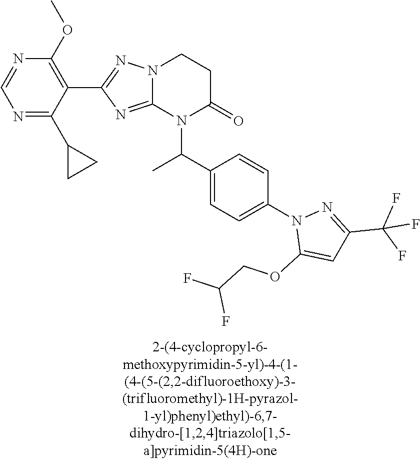 [SMILES: COc1ncnc(C2CC2)c1-c1nc2n(n1)CCC(=O)N2C(C)c1ccc(-n2nc(C(F)(F)F)cc2OCC(F)F)cc1] | ¹H NMR (400 MHz, Chloroform-d) δ 8.61 (s, 1H), 7.65-7.57 (m, 4H), 6.17 (q, J = 7.2 Hz, 1H), 5.99 (s, 1H), 4.45-4.29 (m, 4H), 3.96 (s, 3H), 3.09 (t, J = 7.3 Hz, 2H), 1.98 (d, J = 7.3 Hz, 4H), 1.25- 1.15 (m, 2H), 0.94-0.87 (m, 2H). | m/z: 605.0 [M + H]⁺ |
| 215 | 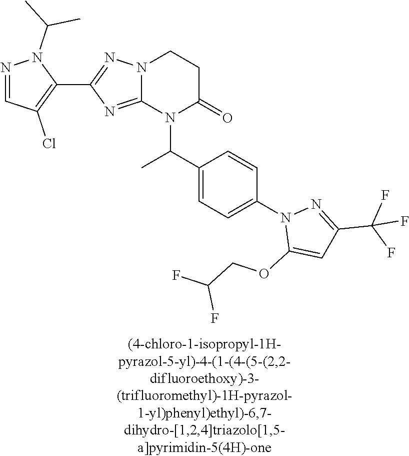 [SMILES: CC(c1ccc(-n2nc(C(F)(F)F)cc2OCC(F)F)cc1)N1C(=O)CCn2nc(-c3c(Cl)cnn3C(C)C)nc21] | ¹H NMR (400 MHz, Chloroform-d) δ 7.62 (s, 4H), 7.51 (s, 1H), 6.24 (q, J = 7.1 Hz, 1H), 6.07 (t, J = 3.9 Hz, 1H), 5.99 (s, 1H), 5.07 (m, 1H), 4.45-4.28 (m, 4H), 3.11 (t, J = 7.3 Hz, 2H), 1.98 (d, J = 7.2 Hz, 3H), 1.48 (d, J = 6.6 Hz, 3H), 1.38 (d, J = 6.6 Hz, 3H). | m/z: 599.3 [M + H]⁺ |
| 216 | 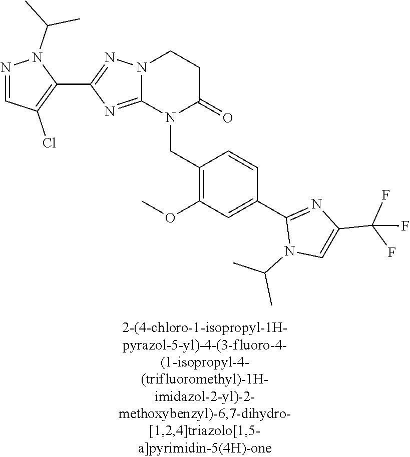 | ¹H NMR (400 MHz, Chloroform-d) δ 7.51 (s, 1H), 7.42 (d, J = 1.4 Hz, 1H), 7.19- 7.10 (m, 2H), 5.28 (s, 2H), 5.19 (m, 1H), 4.45 (t, J = 7.2 Hz, 2H), 4.29-4.18 (m, 1H), 3.98 (d, J = 1.9 Hz, 3H), 3.15 (t, J = 7.3 Hz, 2H), 1.45 (d, J = 6.6 Hz, 6H), 1.40 (d, J = 6.6 Hz, 6H). | m/z: 595.2 [M + H]⁺ |
| 217 | 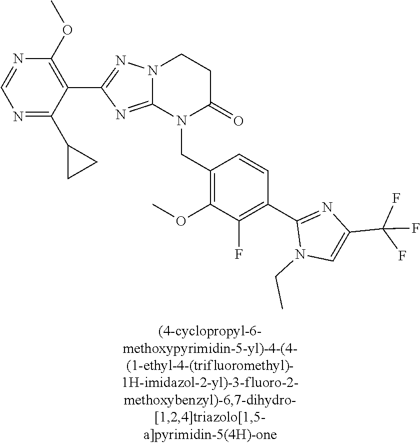 [SMILES: CCn1cc(C(F)(F)F)nc1-c1ccc(CN2C(=O)CCn3nc(-c4c(OC)ncnc4C4CC4)nc32)c(OC)c1F] | ¹H NMR (400 MHz, Chloroform-d) δ 8.61 (s, 1H), 7.40 (s, 1H), 7.15 (d, J = 6.1 Hz, 2H), 5.28 (s, 2H), 4.45 (t, J = 7.3 Hz, 2H), 3.98 (d, J = 2.0 Hz, 3H), 3.95-3.87 (m, 5H), 3.14 (t, J = 7.3 Hz, 2H), 1.99 (dt, J = 8.0, 3.9 Hz, 1H), 1.39 (t, J = 7.3 Hz, 3H), 1.24- 1.16 (m, 2H), 0.93 (dq, J = 7.1, 3.8 Hz, 2H). | m/z: 587.2 [M + H]⁺ |
| 218 | 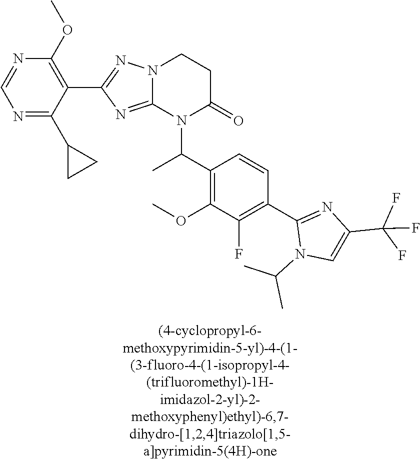 [SMILES: COc1ncnc(C2CC2)c1-c1nc2n(n1)CCC(=O)N2C(C)c1ccc(-c2nc(C(F)(F)F)cn2C(C)C)c(F)c1OC] | ¹H NMR (400 MHz, Chloroform-d) δ 8.62 (s, 1H), 7.54 (d, J = 8.3 Hz, 1H), 7.44- 7.41 (m, 1H), 7.18 (dd, J = 8.1, 6.7 Hz, 1H), 6.21 (q, J = 7.1 Hz, 1H), 4.42-4.31 (m, 2H), 4.26 (m, 1H), 3.96 (s, 3H), 3.86 (d, J = 1.9 Hz, 3H), 3.12-2.92 (m, 2H), 2.02 (td, J = 8.1, 4.1 Hz, 1H), 1.92 (d, J = 7.2 Hz, 3H), 1.42 (t, J = 7.0 Hz, 6H), 1.26-1.19 (m, 2H), 0.95 (ddt, J = 11.8, 8.4, 3.9 Hz, 2H). | m/z: 615.3 [M + H]⁺ |
| 219 | 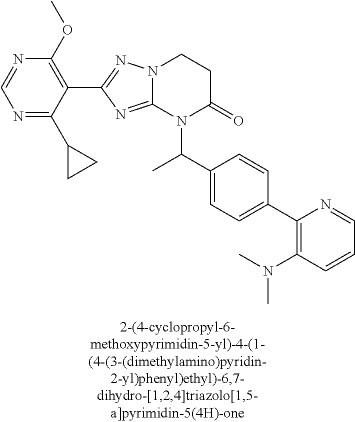 [SMILES: COc1ncnc(C2CC2)c1-c1nc2n(n1)CCC(=O)N2C(C)c1ccc(-c2ncccc2N(C)C)cc1] | ¹H NMR (400 MHz, Chloroform-d) δ 8.59 (s, 1H), 8.24 (d, J = 4.0 Hz, 1H), 7.79 (d, J = 7.9 Hz, 2H), 7.57 (d, J = 8.0 Hz, 2H), 7.32 (d, J = 7.9 Hz, 1H), 7.14 (dd, J = 7.8, 4.4 Hz, 1H), 6.18 (q, J = 7.0 Hz, 1H), 4.37 (dq, J = 13.2, 5.9 Hz, 2H), 3.95 (s, 3H), 3.07 (t, J = 7.1 Hz, 2H), 2.57 (s, 6H), 2.00 (d, J = 7.2 Hz, 4H), 1.15 (d, J = 17.1 Hz, 2H), 0.96- 0.85 (m, 2H). | m/z: 511.1 [M + H]⁺ |
| 220 | 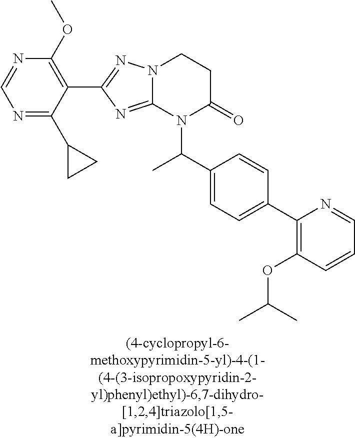 [SMILES: COc1ncnc(C2CC2)c1-c1nc2n(n1)CCC(=O)N2C(C)c1ccc(-c2ncccc2OC(C)C)cc1] | ¹H NMR (400 MHz, Chloroform-d) δ 8.58 (s, 1H), 8.25 (d, J = 4.5 Hz, 1H), 7.91 (d, J = 8.3 Hz, 2H), 7.56 (d, J = 8.3 Hz, 2H), 7.25 (s, 1H), 7.17 (dd, J = 8.3, 4.6 Hz, 1H), 6.19 (q, J = 7.2 Hz, 1H), 4.55 (m, 1H), 4.42-4.32 (m, 2H), 3.94 (s, 3H), 3.08 (t, J = 7.3 Hz, 2H), 2.00 (d, J = 7.2 Hz, 3H), 1.93 (td, J = 8.0, 4.0 Hz, 1H), 1.34 (d, J = 6.1 Hz, 3H), 1.31 (d, J = 6.1 Hz, 3H), 1.16 (dd, J = 7.8, 5.9 Hz, 1H), 1.12-1.06 (m, 1H), 0.91-0.81 (m, 2H). | m/z: 526.3 [M + H]⁺ |

### Example 221. 2-(1-isopropyl-4-methyl-1H-pyrazol-5-yl)-4-(4-(1-methyl-4-(trifluoromethyl)-1H-imidazol-2-yl)benzyl)-4,5,6,7-tetrahydro-[1,2,4]triazolo[1,5-α]pyrimidine

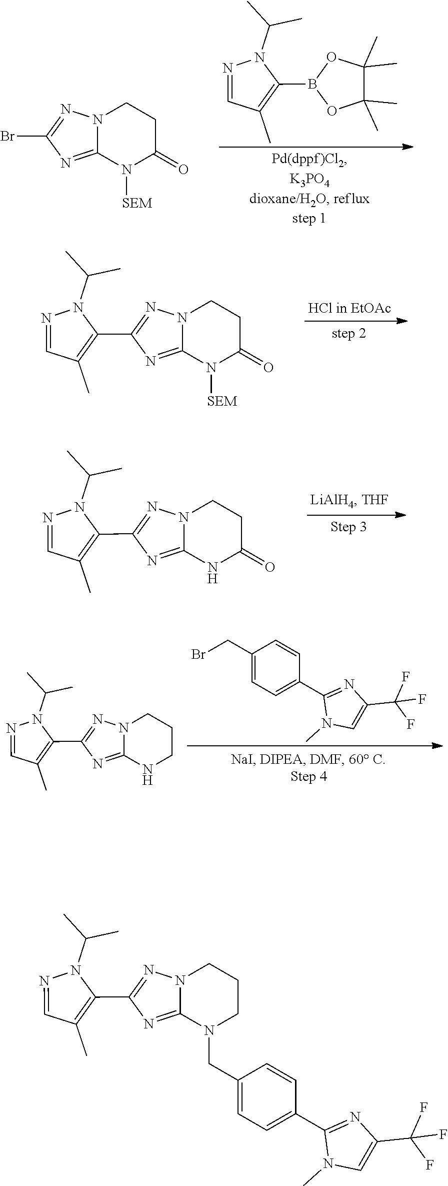

### Step 1. 2-(1-isopropyl-4-methyl-1H-pyrazol-5-yl)-4-((2-(trimethylsilyl)ethoxy)methyl)-6,7-dihydro-[1,2,4]triazolo[1,5-α]pyrimidin-5(4H)-one

2-(1-isopropyl-4-methyl-1H-pyrazol-5-yl)-4-((2-(trimethylsilyl)ethoxy)methyl)-6,7-dihydro-[1,2,4]triazolo[1,5-α]pyrimidin-5(4H)-one was prepared from 2-bromo-4-((2-(trimethylsilyl)ethoxy)methyl)-6,7-dihydro-[1,2,4]triazolo[1,5-α]pyrimidin-5(4H)-one and 1-isopropyl-4-methyl-5-(4,4,5,5-tetramethyl-1,3,2-dioxaborolan-2-yl)-1H-pyrazole following step 3 of Example 1. MS (ESI) m/z 391.2 [M+H]⁺.

### Step 2. 2-(1-isopropyl-4-methyl-1H-pyrazol-5-yl)-6,7-dihydro-[1,2,4]triazolo[1,5-α]pyrimidin-5(4H)-one

2-(1-isopropyl-4-methyl-1H-pyrazol-5-yl)-6,7-dihydro-[1,2,4]triazolo[1,5-α]pyrimidin-5(4H)-one was prepared from 2-(1-isopropyl-4-methyl-1H-pyrazol-5-yl)-4-((2-(trimethylsilyl)ethoxy)methyl)-6,7-dihydro-[1,2,4]triazolo[1,5-α]pyrimidin-5(4H)-one according to step 4 of Example 149. MS (ESI) m/z 261.1 [M+H]⁺.

### Step 3 and step 4. 2-(1-isopropyl-4-methyl-1H-pyrazol-5-yl)-4-(4-(1-methyl-4-(trifluoromethyl)-1H-imidazol-2-yl) benzyl)-4,5,6,7-tetrahydro-[1,2,4]triazolo[1,5-α]pyrimidine

Example 221 2-(1-isopropyl-4-methyl-1H-pyrazol-5-yl)-4-(4-(1-methyl-4-(trifluoromethyl)-1H-imidazol-2-yl) benzyl)-4,5,6,7-tetrahydro-[1,2,4]triazolo[1,5-α]pyrimidine was synthesized from 2-(1-isopropyl-4-methyl-1H-pyrazol-5-yl)-6,7-dihydro-[1,2,4]triazolo[1,5-α]pyrimidin-5(4H)-one according to step 2 and step 3 of Example 52.

Table 15. The compounds listed in Table 15 were synthesized according to Example 221 using the appropriate commercially available reagents and/or intermediates described above.

TABLE 15

| Example | | | |
|---|---|---|---|
| number | Structure | ¹H NMR (δ ppm) | MS (ESI) |
| 221 | 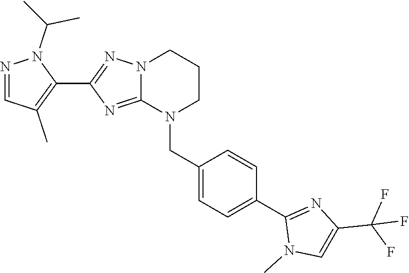 [SMILES: Cc1cnn(C(C)C)c1-c1nc2n(n1)CCCN2Cc1ccc(-c2nc(C(F)(F)F)cn2C)cc1] 2-(1-isopropyl-4-methyl-1H- pyrazol-5-yl)-4-(4-(1-methyl- 4-(trifluoromethyl)-1H- imidazol-2-yl)benzyl)-4,5,6,7- tetrahydro-[1,2,4]triazolo[1,5- a]pyrimidine | ¹H NMR (400 MHz, Chloroform-d) δ 7.63-7.58 (m, 2H), 7.47 (d, J = 8.0 Hz, 2H), 7.38 (s, 1H), 7.31 (d, J = 1.3 Hz, 1H), 5.37-5.31 (m, 1H), 4.74 (s, 2H), 4.13 (t, J = 6.1 Hz, 2H), 3.76 (s, 3H), 3.29-3.21 (m, 2H), 2.28-2.19 (m, 5H), 1.48 (d, J = 6.6 Hz, 6H). | m/z: 485.3 [M + H]⁺ |
| 222 | 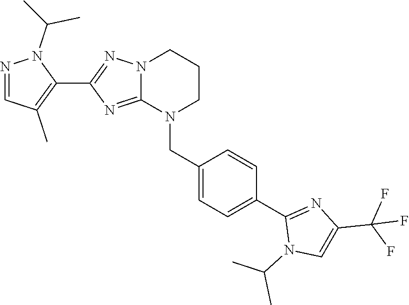 [SMILES: Cc1cnn(C(C)C)c1-c1nc2n(n1)CCCN2Cc1ccc(-c2nc(C(F)(F)F)cn2C(C)C)cc1] 4-(4-(1-isopropyl-4- (trifluoromethyl)-1H- imidazol-2-yl)benzyl)-2-(1- isopropyl-4-methyl-1H- pyrazol-5-yl)-4,5,6,7- tetrahydro-[1,2,4]triazolo[1,5- a]pyrimidine | ¹H NMR (400 MHz, Chloroform-d) δ 7.49 (q, J = 7.8 Hz, 4H), 7.42 (s, 1H), 7.37 (s, 1H), 5.37-5.30 (m, 1H), 4.74 (s, 2H), 4.59-4.52 (m, 1H), 4.13 (t, J = 6.1 Hz, 2H), 3.25 (t, J = 5.5 Hz, 2H), 2.23 (d, J = 10.3 Hz, 5H), 1.47 (dd, J = 10.5, 6.6 Hz, 13H). | m/z: 513.3 [M + H]⁺ |

### Example 223. 2-(4-chloro-1-isopropyl-1H-pyrazol-5-yl)-4-(4-(1-ethyl-4-(trifluoromethyl)-1H-imidazol-2-yl)benzyl)-5-oxo-4,5,6,7-tetrahydropyrazolo[1,5-α]pyrimidine-3-carbonitrile

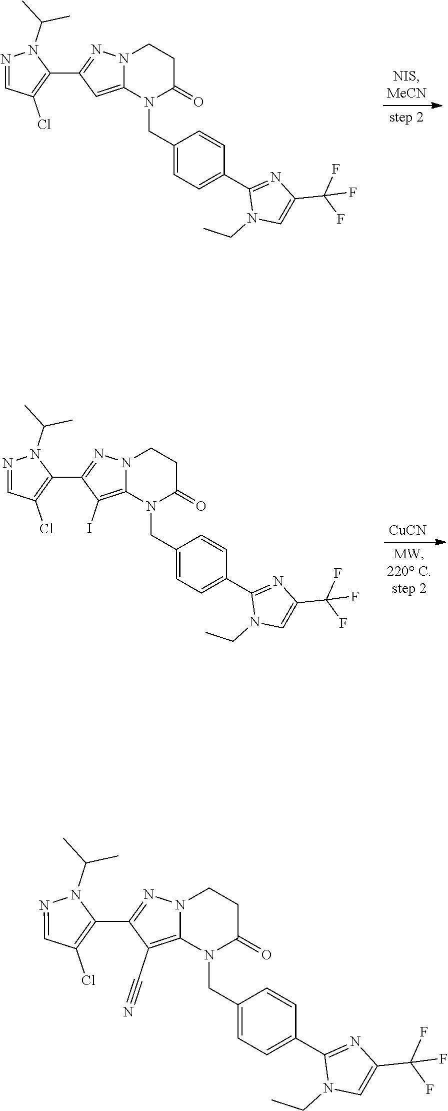 [SMILES: CCn1cc(C(F)(F)F)nc1-c1ccc(CN2C(=O)CCn3nc(-c4c(Cl)cnn4C(C)C)cc32)cc1 ; CCn1cc(C(F)(F)F)nc1-c1ccc(CN2C(=O)CCn3nc(-c4c(Cl)cnn4C(C)C)c(I)c32)cc1 ; "CuCN" ; CCn1cc(C(F)(F)F)nc1-c1ccc(CN2C(=O)CCn3nc(-c4c(Cl)cnn4C(C)C)c(C#N)c32)cc1]

### Step 1. 2-(4-chloro-1-isopropyl-1H-pyrazol-5-yl)-4-(4-(1-ethyl-4-(trifluoromethyl)-1H-imidazol-2-yl)benzyl)-3-iodo-6,7-dihydropyrazolo[1,5-α]pyrimidin-5(4H)-one

To a solution of 2-(4-chloro-1-isopropyl-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one (248 mg, 0.47 mmol) in MeCN (5 mL) was added NIS (315 mg, 1.41 mmol). The resulting solution was heated at 80° C. for 16 h. The reaction mixture was concentrated, diluted with EtOAc and washed with brine. The organic layers were combined, dried over anhydrous Na₂SO₄, concentrated and purified on a Biotage Isolera One (C₁₈ column, eluting with 10% to 95% MeCN/H₂O) to afford 2-(4-chloro-1-isopropyl-1H-pyrazol-5-yl)-4-(4-(1-ethyl-4-(trifluoromethyl)-1H-imidazol-2-yl)benzyl)-3-iodo-6,7-dihydropyrazolo[1,5-α]pyrimidin-5(4H)-one (140 mg). MS (ESI) m/z 658.1 [M+H]⁺.

### Step 2. 2-(4-chloro-1-isopropyl-1H-pyrazol-5-yl)-4-(4-(1-ethyl-4-(trifluoromethyl)-1H-imidazol-2-yl)benzyl)-5-oxo-4,5,6,7-tetrahydropyrazolo[1,5-α]pyrimidine-3-carbonitrile

Into a 5 mL microwave tube purged with nitrogen was added 2-(4-chloro-1-isopropyl-1H-pyrazol-5-yl)-4-(4-(1-ethyl-4-(trifluoromethyl)-1H-imidazol-2-yl)benzyl)-3-iodo-6,7-dihydropyrazolo[1,5-α]pyrimidin-5(4H)-one (80 mg, 0.12 mmol), CuCN (16.5 mg, 0.18 mmol) and NMP (2 mL). The mixture was heated with microwave irradiation for 0.5 h at 220° C. After cooling to ambient temperature, the reaction mixture filtered through celite, concentrated under vacuum and purified by prep-HPLC (condition 2) to afford Example 223 2-(4-chloro-1-isopropyl-1H-pyrazol-5-yl)-4-(4-(1-ethyl-4-(trifluoromethyl)-1H-imidazol-2-yl)benzyl)-5-oxo-4,5,6,7-tetrahydropyrazolo[1,5-α]pyrimidine-3-carbonitrile (18.5 mg). ¹H NMR (400 MHz, Methanol-d₄) δ 7.82 (s, 1H), 7.65-7.52 (m, 5H), 5.40 (s, 2H), 4.57 (d, J=7.0 Hz, 3H), 4.11 (q, J=6.9 Hz, 2H), 3.23 (t, J=6.9 Hz, 2H), 1.41 (d, J=6.4 Hz, 6H), 1.36 (t, J=7.3 Hz, 3H). MS (ESI) m/z 557.20 [M+H]⁺.

### Example 224. 2-(4-chloro-1-isopropyl-1H-pyrazol-5-yl)-4-(4-(5-((methylamino)methyl)-3-(trifluoromethyl)-1H-pyrazol-1-yl)benzyl)-6,7-dihydropyrazolo[1,5-α]pyrimidin-5(4H)-one

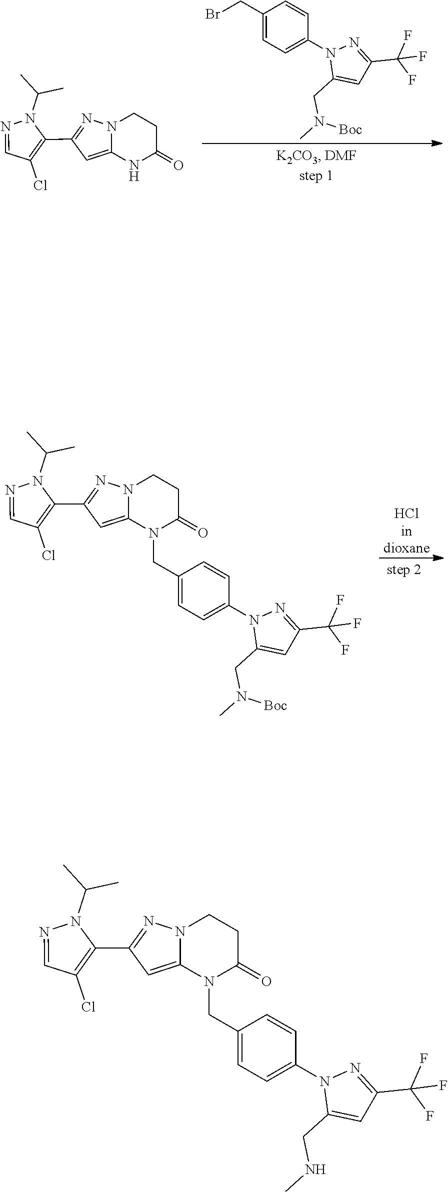 [SMILES: CC(C)n1ncc(Cl)c1-c1cc2n(n1)CCC(=O)N2 ; CN(Cc1cc(C(F)(F)F)nn1-c1ccc(CBr)cc1)C(=O)OC(C)(C)C ; CC(C)n1ncc(Cl)c1-c1cc2n(n1)CCC(=O)N2Cc1ccc(-n2nc(C(F)(F)F)cc2CN(C)C(=O)OC(C)(C)C)cc1 ; "HClindioxane" ; CNCc1cc(C(F)(F)F)nn1-c1ccc(CN2C(=O)CCn3nc(-c4c(Cl)cnn4C(C)C)cc32)cc1]

### Step 1. tert-butyl ((1-(4-((2-(4-chloro-1-isopropyl-1H-pyrazol-5-yl)-5-oxo-6,7-dihydropyrazolo[1,5-α]pyrimidin-4(5H)-yl)methyl)phenyl)-3-(trifluoromethyl)-1H-pyrazol-5-yl)methyl)(methyl)carbamate

tert-butyl ((1-(4-((2-(4-chloro-1-isopropyl-1H-pyrazol-5-yl)-5-oxo-6,7-dihydropyrazolo[1,5-α]pyrimidin-4(5H)-yl) methyl)phenyl)-3-(trifluoromethyl)-1H-pyrazol-5-yl)methyl)(methyl)carbamate was prepared from 2-(4-chloro-1-isopropyl-1H-pyrazol-5-yl)-6,7-dihydropyrazolo[1,5-α]pyrimidin-5(4H)-one and tert-butyl ((1-(4-(bromomethyl)phenyl)-3-(trifluoromethyl)-1H-pyrazol-5-yl)methyl)(methyl)carbamate (intermediate BB-19) according to step 2 of Example 1. MS (ESI) m/z 647.2 [M+H]⁺.

### Step 2. 2-(4-chloro-1-isopropyl-1H-pyrazol-5-yl)-4-(4-(5-((methylamino)methyl)-3-(trifluoromethyl)-1H-pyrazol-1-yl) benzyl)-6,7-dihydropyrazolo[1,5-α]pyrimidin-5(4H)-one

A solution of tert-butyl ((1-(4-((2-(4-chloro-1-isopropyl-1H-pyrazol-5-yl)-5-oxo-6,7-dihydropyrazolo[1,5-α]pyrimidin-4(5H)-yl)methyl)phenyl)-3-(trifluoromethyl)-1H-pyrazol-5-yl)methyl)(methyl)carbamate (50 mg, 0.077 mmol) in HCl dioxane solution (4M, 2 mL) was stirred at room temperature for 1 h before solvent was removed. The crude was purified on a Biotage Isolera One (C₁₈ column, eluting with 10% to 90% MeCN/H₂O containing 0.1% TFA) to afford Example 224 2-(4-chloro-1-isopropyl-1H-pyrazol-5-yl)-4-(4-(5-((methylamino)methyl)-3-(trifluoromethyl)-1H-pyrazol-1-yl)benzyl)-6,7-dihydropyrazolo[1,5-α]pyrimidin-5(4H)-one (24.7 mg).

Table 16. The compounds listed in Table 16 were synthesized according to Example 224 using the appropriate commercially available reagents and/or intermediates described above.

TABLE 16

| Example | | | |
|---|---|---|---|
| number | Structure | ¹H NMR (δ ppm) | MS (ESI) |
| 224 | 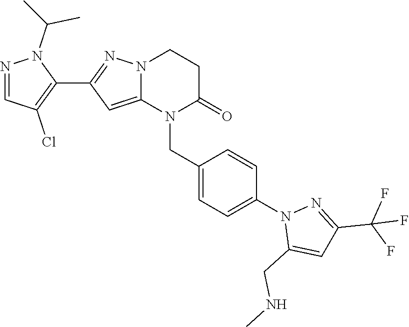 [SMILES: CNCc1cc(C(F)(F)F)nn1-c1ccc(CN2C(=O)CCn3nc(-c4c(Cl)cnn4C(C)C)cc32)cc1] 2-(4-chloro-1-isopropyl-1H- pyrazol-5-yl)-4-(4-(5- ((methylamino)methyl)-3- (trifluoromethyl)-1H-pyrazol- 1-yl)benzyl)-6,7- dihydropyrazolo[1,5- a]pyrimidin-5(4H)-one | ¹H NMR (400 MHz, Methanol-d₄) δ 7.64 (d, J = 8.3 Hz, 2H), 7.55 (d, J = 8.4 Hz, 2H), 7.51 (s, 1H), 7.06 (s, 1H), 6.27 (s, 1H), 5.22-5.09 (m, 3H), 4.49 (t, J = 7.1 Hz, 2H), 4.38 (d, J = 4.9 Hz, 2H), 3.16 (t, J = 7.1 Hz, 2H), 2.70 (s, 3H), 1.41 (d, J = 6.6 Hz, 6H). | m/z: 547.3 [M + H]⁺ |
| 225 | 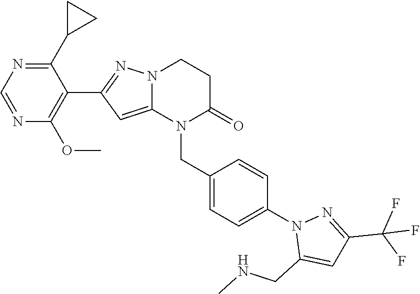 [SMILES: CNCc1cc(C(F)(F)F)nn1-c1ccc(CN2C(=O)CCn3nc(-c4c(OC)ncnc4C4CC4)cc32)cc1] 2-(4-cyclopropyl-6- methoxypyrimidin-5-yl)-4-(4- (5-((methylamino)methyl)-3- (trifluoromethyl)-1H-pyrazol- 1-yl)benzyl)-6,7- dihydropyrazolo[1,5- a]pyrimidin-5(4H)-one | ¹H NMR (400 MHz, Methanol-d₄) δ 8.49 (s, 1H), 7.59 (d, J = 8.6 Hz, 2H), 7.56- 7.50 (m, 2H), 6.84 (s, 1H), 6.02 (s, 1H), 5.14 (s, 2H), 4.45 (t, J = 7.1 Hz, 2H), 3.87 (d, J = 5.5 Hz, 5H), 3.15 (t, J = 7.1 Hz, 2H), 2.40 (s, 3H), 2.16-2.09 (m, 1H), 2.04 (s, 1H), 1.11-1.07 (m, 2H), 0.93- 0.88 (m, 2H). | m/z: 553.2 [M + H]⁺ |

### Example 226. 2-(4-chloro-1-isopropyl-1H-pyrazol-5-yl)-4-(4-(pyridin-2-ylmethoxy)benzyl)-6,7-dihydropyrazolo[1,5-α]pyrimidin-5(4H)-one

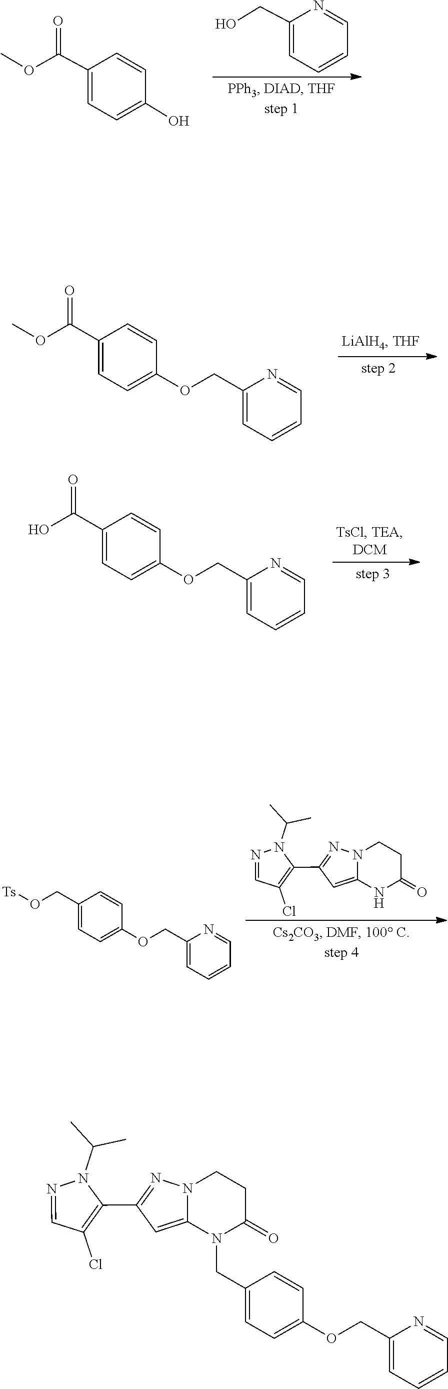

### Step 1. methyl 4-(pyridin-2-ylmethoxy)benzoate

To a stirred solution of methyl 4-hydroxybenzoate (300 mg, 1.97 mmol), pyridin-2-ylmethanol (215 mg, 1.97 mmol), and PPh₃ (622 mg, 2.37 mmol) in THF (5 mL) was added diisopropyl azodicarboxylate (478 mg, 2.37 mmol) at room temperature. The resulting solution was stirred at room temperature for 3 h. The solution was concentrated under vacuum. The crude product was purified by silica gel chromatography (eluting with 1/5 EtOAc/PE) to afford methyl 4-(pyridin-2-ylmethoxy)benzoate (540 mg). MS (ESI) m/z 244.1 [M+H]⁺.

### Step 2. (4-(pyridin-2-ylmethoxy)phenyl)methanol

(4-(pyridin-2-ylmethoxy)phenyl)methanol was prepared from methyl 4-(pyridin-2-ylmethoxy)benzoate according to step 3 of intermediate BB-1. MS (ESI) m/z 216.1 [M+H]⁺.

### Step 3. 4-(pyridin-2-ylmethoxy)benzyl 4-methylbenzenesulfonate

To a solution of (4-(pyridin-2-ylmethoxy)phenyl)methanol (100 mg, 0.46 mmol), and TEA (56.5 mg, 0.56 mmol) in DCM (2 mL) was added p-toluenesulfonyl chloride (106 mg, 0.56 mmol) at room temperature. The resulting solution was stirred at room temperature for 2 h. The solution was concentrated under vacuum. The crude product was purified by silica gel chromatography (eluting with 1/20 EtOAc/PE) to afford 4-(pyridin-2-ylmethoxy)benzyl 4-methylbenzenesulfonate (103 mg). MS (ESI) m/z 370.1 [M+H]⁺.

### Step 4. 2-(4-chloro-1-isopropyl-1H-pyrazol-5-yl)-4-(4-(pyridin-2-ylmethoxy)benzyl)-6,7-dihydropyrazolo[1,5-α]pyrimidin-5(4H)-one

A mixture of 2-(4-chloro-1-isopropyl-1H-pyrazol-5-yl)-6,7-dihydropyrazolo[1,5-α]pyrimidin-5(4H)-one (75.6 mg, 0.27 mmol), 4-(pyridin-2-ylmethoxy)benzyl 4-methylbenzenesulfonate (100 mg, 0.27 mmol) and Cs₂CO₃ (176.4 mg, 0.54 mmol) in DMF (5 mL) was heated at 100° C. overnight. The reaction mixture was diluted with EtOAc (20 mL), washed with water and brine, dried over anhydrous Na₂SO₄, concentrated and purified by prep-HPLC (condition 4) to afford Example 226 2-(4-chloro-1-isopropyl-1H-pyrazol-5-yl)-4-(4-(pyridin-2-ylmethoxy)benzyl)-6,7-dihydropyrazolo[1,5α]pyrimidin-5(4H)-one (24.2 mg). ¹H NMR (400 MHz, Chloroform-d) δ 8.59 (d, J=4.8 Hz, 1H), 7.73 (t, J=7.4 Hz, 1H), 7.51 (d, J=14.6 Hz, 2H), 7.28 (d, J=8.8 Hz, 2H), 7.23 (s, 1H), 6.94 (d, J=8.7 Hz, 2H), 6.17 (s, 1H), 5.24-5.11 (m, 3H), 4.92 (s, 2H), 4.39 (t, J=7.1 Hz, 2H), 3.06 (t, J=7.1 Hz, 2H), 1.44 (d, J=6.6 Hz, 6H). MS (ESI) m/z 477.2 [M+H]⁺.

### Example 227. 2-(4-chloro-1-(1-hydroxypropan-2-yl)-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one

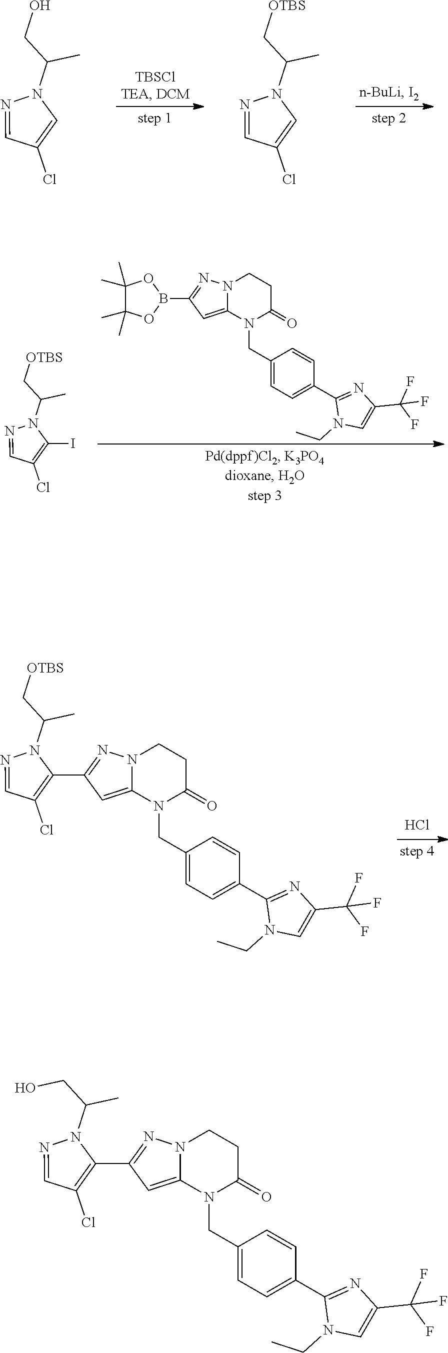

### Step 1. 1-(1-((tert-butyldimethylsilyl)oxy)propan-2-yl)-4-chloro-1H-pyrazole

To a solution of 2-(4-chloro-1H-pyrazol-1-yl)propan-1-ol (1.6 g, 10 mmol, prepared in step 3 of Intermediate 15) and TEA (3.04 g, 30 mmol) in dry DCM (20 mL) was added tert-Butyldimethylsilyl chloride (1.81 g, 12 mmol) in dry DCM (10 mL) slowly at 0° C. After addition, the resulting mixture was stirred at room temperature overnight. The reaction mixture was diluted with DCM (30 mL), washed with water and brine, dried over anhydrous Na₂SO₄, concentrated and purified on a Biotage Isolera One (C₁₈ column, eluting with 10% to 90% MeCN/H₂O) to give 1-(1-((tert-butyldimethylsilyl)oxy)propan-2-yl)-4-chloro-1H-pyrazole (742 mg). MS (ESI) m/z 275.1 [M+H]⁺.

### Step 2. 1-(1-((tert-butyldimethylsilyl)oxy)propan-2-yl)-4-chloro-5-iodo-1H-pyrazole

1-(1-((tert-butyldimethylsilyl)oxy)propan-2-yl)-4-chloro-5-iodo-1H-pyrazole was prepared from 1-(1-((tert-butyldimethylsilyl)oxy)propan-2-yl)-4-chloro-1H-pyrazole according to step 2 of intermediate 14. MS (ESI) m/z 401.0 [M+H]⁺.

### Step 3. 2-(1-(1-((tert-butyldimethylsilyl)oxy)propan-2-yl)-4-chloro-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one

2-(1-(1-((tert-butyldimethylsilyl)oxy)propan-2-yl)-4-chloro-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one was prepared from 4-(4-(1-ethyl-4-(trifluoromethyl)-1H-imidazol-2-yl)benzyl)-2-(4,4,5, 5-tetramethyl-1,3,2-dioxaborolan-2-yl)-6,7-dihydropyrazolo[1,5-α]pyrimidin-5(4H)-one (synthesized in step 2 of Example 154) and 1-(1-((tert-butyldimethylsilyl)oxy)propan-2-yl)-4-chloro-5-iodo-1H-pyrazole following step 3 of Example 149. MS (ESI) m/z 662.3 [M+H]⁺.

### Step 4. 2-(4-chloro-1-(1-hydroxypropan-2-yl)-1H-pyrazol-5-yl)-4-(4-(1-ethyl-4-(trifluoromethyl)-1H-imidazol-2-yl) benzyl)-6,7-dihydropyrazolo[1,5-α]pyrimidin-5(4H)-one

A solution of 2-(1-(1-((tert-butyldimethylsilyl)oxy)propan-2-yl)-4-chloro-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one (180 mg, 0.27 mmol) in a mixture of THF (5 mL) and aqueous HCl (2 M, 15 mL) was stirred at room temperature for 2 h. The solvent was removed and the crude residue purified on a Biotage Isolera One (C₁₈ column, eluting with 10% to 90% MeCN/H₂O) to give Example 227 2-(4-chloro-1-(1-hydroxypropan-2-yl)-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one (42.8 mg). ¹H NMR (400 MHz, Chloroform-d) δ 7.60 (d, J=8.0 Hz, 2H), 7.51 (d, J=8.0 Hz, 2H), 7.35 (s, 2H), 5.36-5.28 (m, 1H), 5.18 (s, 2H), 4.38 (t, J=7.3 Hz, 2H), 4.03 (q, J=7.3 Hz, 2H), 3.89 (s, 3H), 3.07 (t, J=7.3 Hz, 2H), 1.49 (d, J=6.6 Hz, 6H), 1.41 (t, J=7.3 Hz, 3H). MS (ESI) m/z 548.2 [M+H]⁺.

### Example 228. 2-(4-chloro-1-isopropyl-1H-pyrazol-5-yl)-4-(3-(2-hydroxyethoxy)-4-(1-isopropyl-4-(trifluoromethyl)-1H-imidazol-2-yl)benzyl)-6,7-dihydro-[1,2,4]triazolo[1,5-α]pyrimidin-5(4H)-one

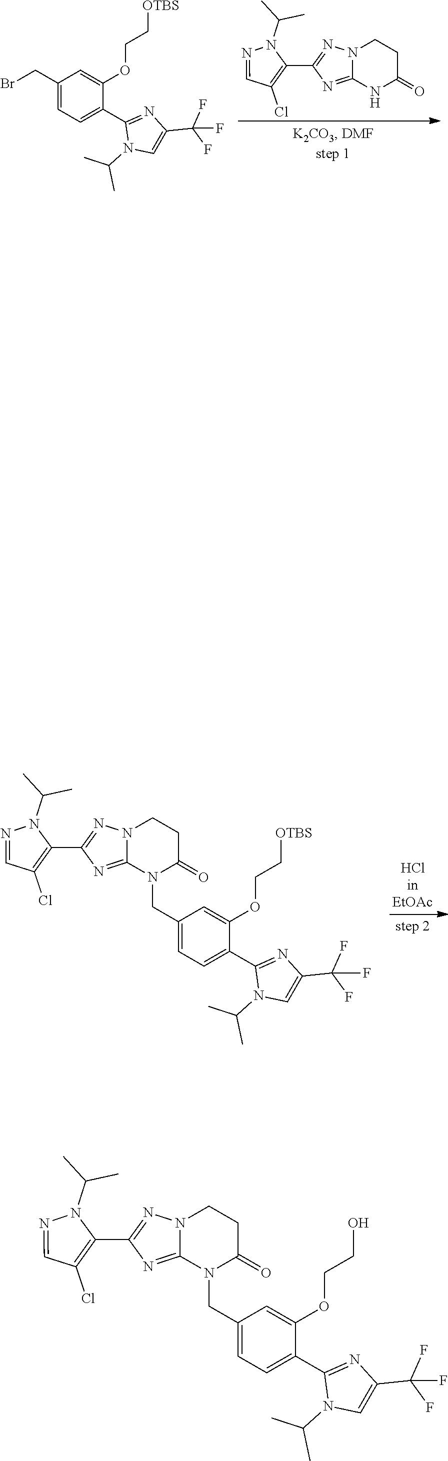 [SMILES: CC(C)n1cc(C(F)(F)F)nc1-c1ccc(CBr)cc1OCCO[Si](C)(C)C(C)(C)C ; CC(C)n1ncc(Cl)c1-c1nc2n(n1)CCC(=O)N2 ; CC(C)n1cc(C(F)(F)F)nc1-c1ccc(CN2C(=O)CCn3nc(-c4c(Cl)cnn4C(C)C)nc32)cc1OCCO[Si](C)(C)C(C)(C)C ; "HClinEtOAc" ; CC(C)n1cc(C(F)(F)F)nc1-c1ccc(CN2C(=O)CCn3nc(-c4c(Cl)cnn4C(C)C)nc32)cc1OCCO]

### Step 1. 4-(3-(2-((tert-butyldimethylsilyl)oxy)ethoxy)-4-(1-isopropyl-4-(trifluoromethyl)-1H-imidazol-2-yl)benzyl)-2-(4-chloro-1-isopropyl-1H-pyrazol-5-yl)-6,7-dihydro-[1,2,4]triazolo[1,5-α]pyrimidin-5(4H)-one

4-(3-(2-((tert-butyldimethylsilyl)oxy)ethoxy)-4-(1-isopropyl-4-(trifluoromethyl)-1H-imidazol-2-yl)benzyl)-2-(4-chloro-1-isopropyl-1H-pyrazol-5-yl)-6,7-dihydro-[1,2,4]triazolo[1,5-α]pyrimidin-5(4H)-one was prepared from 2-(4-(bromomethyl)-2-(2-((tert-butyldimethylsilyl)oxy)ethoxy)phenyl)-1-isopropyl-4-(trifluoromethyl)-1H-imidazole and 2-(4-chloro-1-isopropyl-1H-pyrazol-5-yl)-6,7-dihydro-[1,2,4]triazolo[1,5-α]pyrimidin-5(4H)-one (prepared following step 2 and 3 of Example 164) following step 2 of Example 1. MS (ESI) m/z 721.3 [M+H]⁺.

### Step 2. 2-(4-chloro-1-isopropyl-1H-pyrazol-5-yl)-4-(3-(2-hydroxyethoxy)-4-(1-isopropyl-4-(trifluoromethyl)-1H-imidazol-2-yl)benzyl)-6,7-dihydro-[1,2,4]triazolo[1,5-α]pyrimidin-5(4H)-one

A solution of 4-(3-(2-((tert-butyldimethylsilyl)oxy)ethoxy)-4-(1-isopropyl-4-(trifluoromethyl)-1H-imidazol-2-yl)benzyl)-2-(4-chloro-1-isopropyl-1H-pyrazol-5-yl)-6,7-dihydro-[1,2,4]triazolo[1,5-α]pyrimidin-5(4H)-one (54 mg, 0.075 mmol) in HCl EtOAc solution (4M, 5 mL) was stirred at room temperature for 1 h before solvent was removed. The crude residue was purified by prep-HPLC (condition 2) to afford Example 228 2-(4-chloro-1-isopropyl-1H-pyrazol-5-yl)-4-(3-(2-hydroxyethoxy)-4-(1-isopropyl-4-(trifluoromethyl)-1H-imidazol-2-yl)benzyl)-6,7-dihydro-[1,2,4]triazolo[1,5-α]pyrimidin-5(4H)-one (12.1 mg). ¹H NMR (400 MHz, Chloroform-d) δ 7.55 (s, 1H), 7.42-7.37 (m, 1H), 7.33 (d, J=1.1 Hz, 1H), 7.28 (dd, J=7.8, 1.3 Hz, 1H), 7.21 (d, J=7.8 Hz, 1H), 5.28-5.21 (m, 1H), 5.16 (s, 2H), 4.42 (t, J=7.4 Hz, 2H), 4.36-4.29 (m, 1H), 4.21-4.16 (m, 2H), 3.77-3.72 (m, 2H), 3.13 (t, J=7.4 Hz, 2H), 1.50 (d, J=6.6 Hz, 6H), 1.39 (d, J=6.7 Hz, 6H). MS (ESI) m/z 607.2 [M+H]⁺.

### Example 229. 3-amino-2-(4-chloro-1-isopropyl-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one

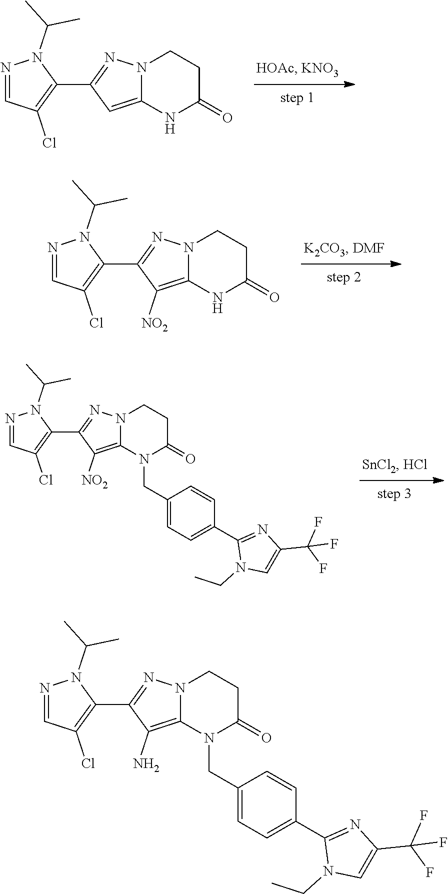

### Step 1. 2-(4-chloro-1-isopropyl-1H-pyrazol-5-yl)-3-nitro-6,7-dihydropyrazolo[1,5-α]pyrimidin-5(4H)-one

To a solution of 2-(4-chloro-1-isopropyl-1H-pyrazol-5-yl)-6,7-dihydropyrazolo[1,5-α]pyrimidin-5(4H)-one (200 mg, 0.71 mmol) in H₂SO₄ (10 mL) was added fuming HNO₃ (1.5 mL) at 0° C. dropwise. The mixture was then stirred at room temperature for 2 h then quenched with NaHCO₃ and extracted with EtOAc. The organic phase was washed with brine, dried over anhydrous Na₂SO₄ and concentrated under vacuum to give 2-(4-chloro-1-isopropyl-1H-pyrazol-5-yl)-3-nitro-6,7-dihydropyrazolo[1,5-α]pyrimidin-5(4H)-one (200 mg) which was used directly in next step without further purification. MS (ESI) m/z 325.1 [M+H]⁺.

### Step 2. 2-(4-chloro-1-isopropyl-1H-pyrazol-5-yl)-4-(4-(1-ethyl-4-(trifluoromethyl)-1H-imidazol-2-yl)benzyl)-3-nitro-6,7-dihydropyrazolo[1,5-α]pyrimidin-5(4H)-one

2-(4-chloro-1-isopropyl-1H-pyrazol-5-yl)-4-(4-(1-ethyl-4-(trifluoromethyl)-1H-imidazol-2-yl)benzyl)-3-nitro-6,7-dihydropyrazolo[1,5-α]pyrimidin-5(4H)-one was prepared from 2-(4-chloro-1-isopropyl-1H-pyrazol-5-yl)-3-nitro-6,7-dihydropyrazolo[1,5-α]pyrimidin-5(4H)-one following step 2 of Example 1. MS (ESI) m/z 577.2 [M+H]⁺.

### Step 3. 3-amino-2-(4-chloro-1-isopropyl-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one

To a solution of 2-(4-chloro-1-isopropyl-1H-pyrazol-5-yl)-4-(4-(1-ethyl-4-(trifluoromethyl)-1H-imidazol-2-yl) benzyl)-3-nitro-6,7-dihydropyrazolo[1,5-α]pyrimidin-5(4H)-one (100 mg,0.17 mmol) in EtOH (5 mL) was added stannous chloride dihydrate (137 mg, 0.61 mmol) and concentrated HCl (100 uL). The resulting mixture was heated at 40° C. for 3 h. The solvent was removed and the crude was purified with prep-TLC (PE/EtOAc=1/1) to give Example 229 3-amino-2-(4-chloro-1-isopropyl-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one (19.3 mg). ¹H NMR (400 MHz, Chloroform-d) δ 7.54 (d, J=8.2 Hz, 3H), 7.45 (d, J=8.0 Hz, 2H), 7.36 (s, 1H), 5.43 (s, 2H), 4.65-4.58 (m, 1H), 4.36 (t, J=6.8 Hz, 2H), 4.04 (q, J=7.3 Hz, 2H), 3.04 (t, J=6.8 Hz, 2H), 1.43 (d, J=6.2 Hz, 9H). MS (ESI) m/z 547.2 [M+H]⁺.

### Example 230. 2-(4-chloro-1-isopropyl-1H-pyrazol-5-yl)-4-(4-(3-morpholino-1H-pyrazol-1-yl)benzyl)-6,7-dihydropyrazolo[1,5-α]pyrimidin-5(4H)-one

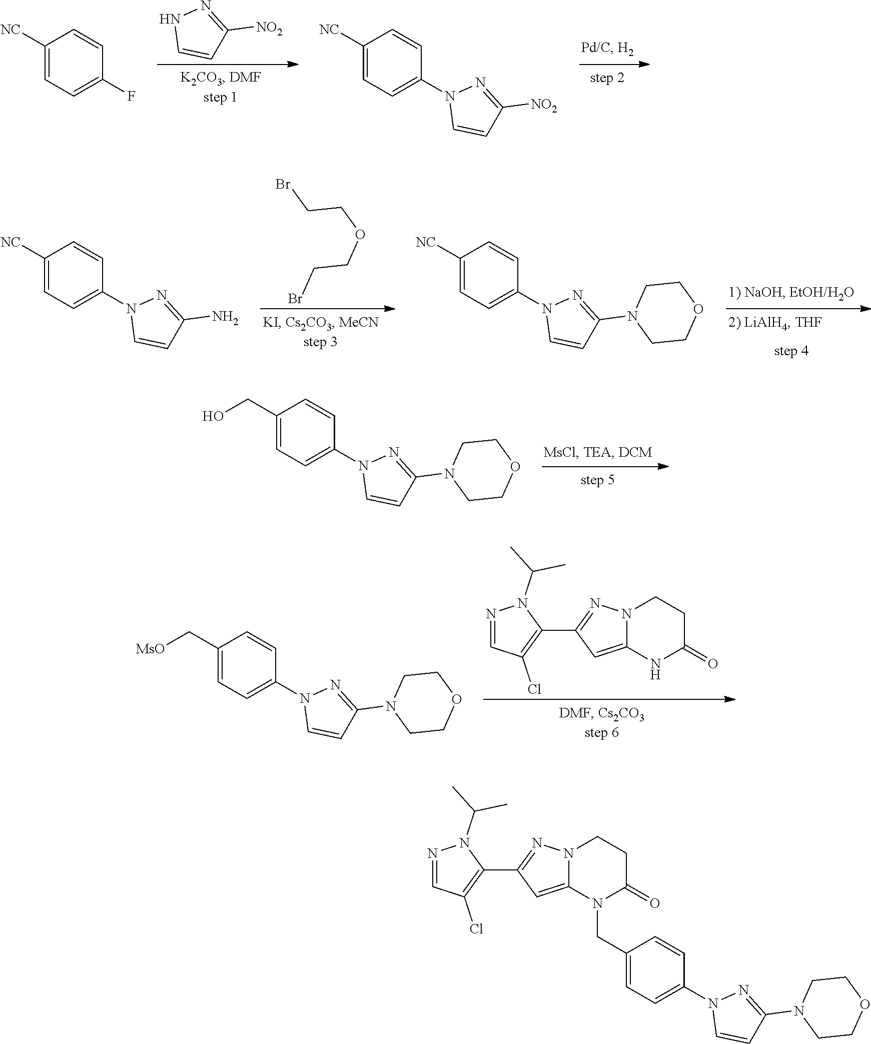

### Step 1. 4-(3-nitro-1H-pyrazol-1-yl)benzonitrile

A mixture of 3-nitro-1H-pyrazole (2.8 g, 24.8 mmol), 4-fluorobenzonitrile (3 g, 24.8 mmol) and potassium carbonate (6.8 g, 49.6 mmol) in DMF (30 mL) was heated at 110° C. for 16 h. After cooling to ambient temperature, the reaction mixture was poured into water (500 mL) and the resulting solids were collected by filtration and dried under vacuum and purified on a Biotage Isolera One (C₁₈ column, eluting with 10% to 90% MeCN/H₂O) to afford 4-(3-nitro-1H-pyrazol-1-yl)benzonitrile (2.6 g). ¹H NMR (400 MHz, DMSO-d₆) δ 8.90 (d, J=2.8 Hz, 1H), 8.14 (d, J=8.8 Hz, 2H), 8.08 (d, J=8.8 Hz, 2H), 7.40 (d, J=2.8 Hz, 1H).

### Step 2. 4-(3-amino-1H-pyrazol-1-yl)benzonitrile

A mixture of 4-(3-nitro-1H-pyrazol-1-yl)benzonitrile (2 g, 9.3 mmol) and Pd/C (5%, 400 mg) was stirred at room temperature for 2 h under an atmosphere of H₂. The reaction mixture was filtered through celite and concentrated under vacuum to afford 4-(3-amino-1H-pyrazol-1-yl)benzonitrile (1.7 g). MS (ESI) m/z 185.1 [M+H]⁺.

### Step 3. 4-(3-morpholino-1H-pyrazol-1-yl)benzonitrile

A mixture of 4-(3-amino-1H-pyrazol-1-yl)benzonitrile (1.7 g, 9.2 mmol), 1-bromo-2-(2-bromoethoxy)ethane (4.3 g, 18.4 mmol), Cs₂CO₃ (8.9 g, 27.6 mmol, 3.0 eq) and KI (7.6 g, 45.9 mmol, 5.0 eq) in MeCN (17 mL) was heated at 80° C. for 12 h. The reaction mixture concentrated and purified by column chromatography on silica gel (eluting with EtOAc/PE 1/2) to afford 4-(3-morpholino-1H-pyrazol-1-yl)benzonitrile (850 mg). MS (ESI) m/z 255.1 [M+H]⁺.

### Step 4. (4-(3-morpholino-1H-pyrazol-1-yl)phenyl)methanol

(4-(3-morpholino-1H-pyrazol-1-yl)phenyl)methanol was prepared from 4-(3-morpholino-1H-pyrazol-1-yl)benzonitrile according to step 3 of intermediate BB-4. MS (ESI) m/z 260.1 [M+H]⁺.

### Step 5 and step 6. 2-(4-chloro-1-isopropyl-1H-pyrazol-5-yl)-4-(4-(3-morpholino-1H-pyrazol-1-yl)benzyl)-6,7-dihydropyrazolo[1,5-α]pyrimidin-5(4H)-one

Example 230 2-(4-chloro-1-isopropyl-1H-pyrazol-5-yl)-4-(4-(3-morpholino-1H-pyrazol-1-yl)benzyl)-6,7-dihydropyrazolo[1,5-α]pyrimidin-5(4H)-one was prepared from (4-(3-morpholino-1H-pyrazol-1-yl)phenyl)methanol and methanesulfonyl chloride following step 3 and step 4 of Example 226. ¹H NMR (400 MHz, Chloroform-d) δ 7.73 (d, J=2.6 Hz, 1H), 7.57-7.53 (m, 2H), 7.48 (s, 1H), 7.40-7.34 (m, 2H), 6.17 (s, 1H), 5.90 (d, J=2.6 Hz, 1H), 5.20-5.10 (m, 1H), 4.98 (s, 2H), 4.40 (t, J=7.1 Hz, 2H), 3.86-3.79 (m, 4H), 3.29-3.22 (m, 4H), 3.07 (t, J=7.1 Hz, 2H), 1.43 (d, J=6.7 Hz, 6H). MS (ESI) m/z 521.2 [M+H]⁺.

### Example 231. 2-(4-chloro-1-isopropyl-1H-pyrazol-5-yl)-6-hydroxy-4-(4-(1-methyl-4-(trifluoromethyl)-1H-imidazol-2-yl)benzyl)-6,7-dihydropyrazolo[1,5-α]pyrimidin-5(4H)-one

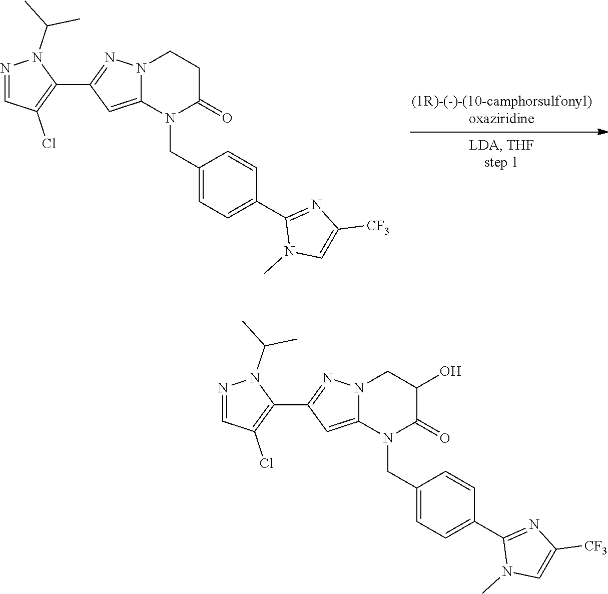 [SMILES: CC(C)n1ncc(Cl)c1-c1cc2n(n1)CCC(=O)N2Cc1ccc(-c2nc(C(F)(F)F)cn2C)cc1 ; CC(C)n1ncc(Cl)c1-c1cc2n(n1)CC(O)C(=O)N2Cc1ccc(-c2nc(C(F)(F)F)cn2C)cc1]

### Step 1. 2-(4-chloro-1-isopropyl-1H-pyrazol-5-yl)-6-hydroxy-4-(4-(1-methyl-4-(trifluoromethyl)-1H-imidazol-2-yl)benzyl)-6,7-dihydropyrazolo[1,5-α]pyrimidin-5(4H)-one

To a solution of 2-(4-chloro-1-isopropyl-1H-pyrazol-5-yl)-4-(4-(1-methyl-4-(trifluoromethyl)-1H-imidazol-2-yl) benzyl)-6,7-dihydropyrazolo[1,5-α]pyrimidin-5(4H)-one (87.5 mg, 0.17 mmol) in dry THF (5 mL) was added LDA (2 M, 0.13 mL, 0.26 mmol) at −78° C. under an atmosphere of N₂. The mixture was stirred at −78° C. for 30 min before (1R)-(-)-(10-camphorsulfonyl) oxaziridine (58.2 mg, 0.26 mmol) in dry THF (2 mL) was added. The reaction mixture was stirred at −78° C. for 30 min then allowed to warm to room temperature and stirring continued for 30 min. The mixture was quenched with MeOH (2 mL), then concentrated under vacuum and purified by Biotage Isolera One (C₁₈ column, eluting with 10% to 90% MeCN/H₂O) to afford Example 231 2-(4-chloro-1-isopropyl-1H-pyrazol-5-yl)-6-hydroxy-4-(4-(1-methyl-4-(trifluoromethyl)-1H-imidazol-2-yl)benzyl)-6,7-dihydropyrazolo[1,5-α]pyrimidin-5(4H)-one (15.5 mg). ¹H NMR (400 MHz, Chloroform-d) δ 7.62 (d, J=8.1 Hz, 2H), 7.48 (s, 1H), 7.44 (d, J=8.1 Hz, 2H), 7.30 (s, 1H), 6.24 (s, 1H), 5.24-5.12 (m, 1H), 5.08 (s, 2H), 4.82 (dd, J=12.4, 7.5 Hz, 1H), 4.68 (dd, J=11.9, 7.5 Hz, 1H), 4.14 (t, J=12.2 Hz, 1H), 3.75 (s, 3H), 2.00 (s, 3H), 1.45 (dd, J=6.6, 2.9 Hz, 6H). MS (ESI) m/z 534.2 [M+H]⁺.

### Example 232. 2-(4-chloro-1-isopropyl-1H-pyrazol-5-yl)-6-hydroxy-6-methyl-4-(4-(1-methyl-4-(trifluoromethyl)-1H-imidazol-2-yl)benzyl)-6,7-dihydropyrazolo[1,5-α]pyrimidin-5(4H)-one

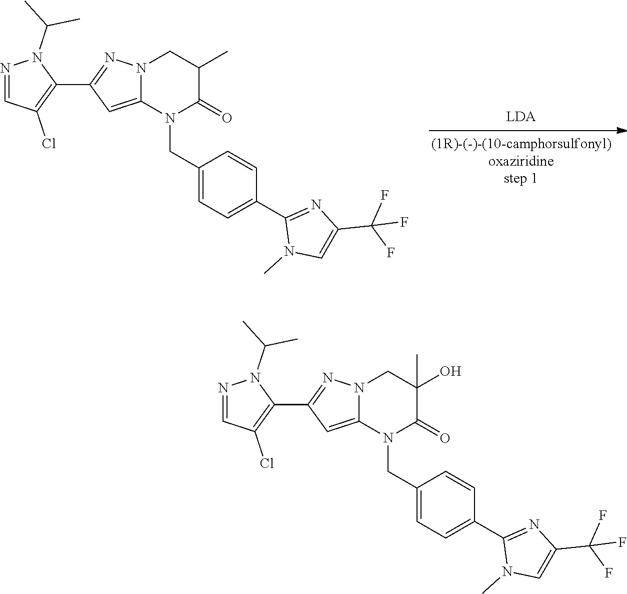 [SMILES: CC1Cn2nc(-c3c(Cl)cnn3C(C)C)cc2N(Cc2ccc(-c3nc(C(F)(F)F)cn3C)cc2)C1=O ; CC(C)n1ncc(Cl)c1-c1cc2n(n1)CC(C)(O)C(=O)N2Cc1ccc(-c2nc(C(F)(F)F)cn2C)cc1]

### Step 1. 2-(4-chloro-1-isopropyl-1H-pyrazol-5-yl)-6-hydroxy-6-methyl-4-(4-(1-methyl-4-(trifluoromethyl)-1H-imidazol-2-yl)benzyl)-6,7-dihydropyrazolo[1,5-α]pyrimidin-5(4H)-one

Example 232 2-(4-chloro-1-isopropyl-1H-pyrazol-5-yl)-6-hydroxy-6-methyl-4-(4-(1-methyl-4-(trifluoromethyl)-1H-imidazol-2-yl)benzyl)-6,7-dihydropyrazolo[1,5-α]pyrimidin-5(4H)-one was prepared from 2-(4-chloro-1-isopropyl-1H-pyrazol-5-yl)-6-methyl-4-(4-(1-methyl-4-(trifluoromethyl)-1H-imidazol-2-yl)benzyl)-6,7-dihydropyrazolo[1,5-α]pyrimidin-5(4H)-one (synthesized according to Example 31) following Example 231. ¹H NMR (400 MHz, Chloroform-d) δ 7.65-7.59 (m, 2H), 7.48 (s, 1H), 7.45-7.40 (m, 2H), 7.30 (d, J=1.3 Hz, 1H), 6.24 (s, 1H), 5.26-5.19 (m, 1H), 5.12 (d, J=15.4 Hz, 1H), 5.02 (d, J=15.4 Hz, 1H), 4.46 (d, J=12.9 Hz, 1H), 4.27 (d, J=13.0 Hz, 1H), 3.91 (d, J=7.6 Hz, 1H), 3.75 (s, 3H), 1.50-1.42 (m, 9H). MS (ESI) m/z 548.2 [M+H]⁺.

### Example 233. 6-amino-2-(4-chloro-1-isopropyl-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one

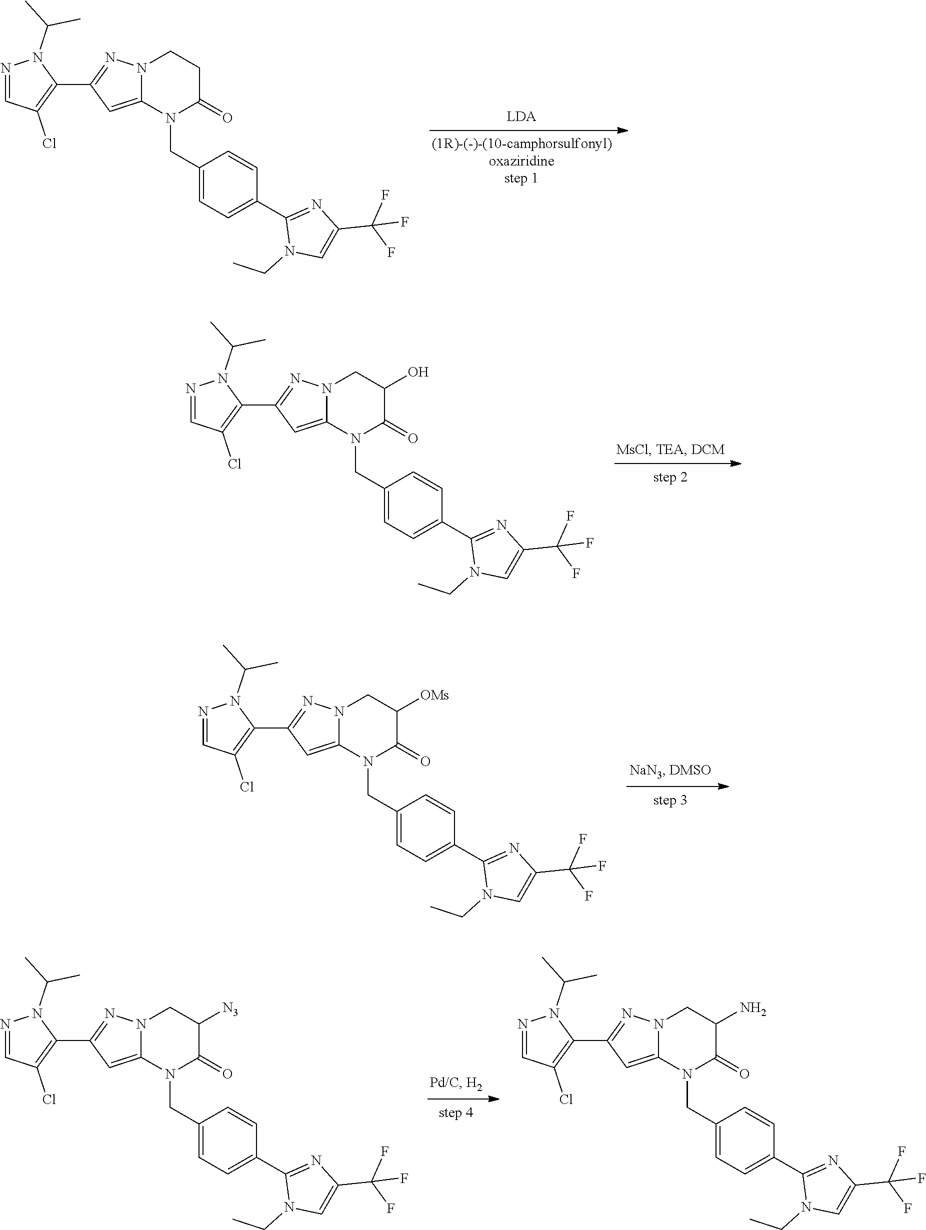 [SMILES: CCn1cc(C(F)(F)F)nc1-c1ccc(CN2C(=O)CCn3nc(-c4c(Cl)cnn4C(C)C)cc32)cc1 ; CCn1cc(C(F)(F)F)nc1-c1ccc(CN2C(=O)C(O)Cn3nc(-c4c(Cl)cnn4C(C)C)cc32)cc1 ; CCn1cc(C(F)(F)F)nc1-c1ccc(CN2C(=O)C(OS(C)(=O)=O)Cn3nc(-c4c(Cl)cnn4C(C)C)cc32)cc1 ; CCn1cc(C(F)(F)F)nc1-c1ccc(CN2C(=O)C(N=[N+]=[N-])Cn3nc(-c4c(Cl)cnn4C(C)C)cc32)cc1 ; CCn1cc(C(F)(F)F)nc1-c1ccc(CN2C(=O)C(N)Cn3nc(-c4c(Cl)cnn4C(C)C)cc32)cc1]

### Step 1. 2-(4-chloro-1-isopropyl-1H-pyrazol-5-yl)-4-(4-(1-ethyl-4-(trifluoromethyl)-1H-imidazol-2-yl)benzyl)-6-hydroxy-6,7-dihydropyrazolo[1,5-α]pyrimidin-5(4H)-one

2-(4-chloro-1-isopropyl-1H-pyrazol-5-yl)-4-(4-(1-ethyl-4-(trifluoromethyl)-1H-imidazol-2-yl)benzyl)-6-hydroxy-6,7-dihydropyrazolo[1,5-α]pyrimidin-5(4H)-one was prepared from 2-(4-chloro-1-isopropyl-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one following Example 231. MS (ESI) m/z 548.2 [M+H]⁺.

### Step 2. 2-(4-chloro-1-isopropyl-1H-pyrazol-5-yl)-4-(4-(1-ethyl-4-(trifluoromethyl)-1H-imidazol-2-yl)benzyl)-5-oxo-4,5,6,7-tetrahydropyrazolo[1,5-α]pyrimidin-6-yl methanesulfonate

To a solution of 2-(4-chloro-1-isopropyl-1H-pyrazol-5-yl)-4-(4-(1-ethyl-4-(trifluoromethyl)-1H-imidazol-2-yl)benzyl)-6-hydroxy-6,7-dihydropyrazolo[1,5-α]pyrimidin-5(4H)-one (350 mg, 0.64 mmol), triethylamine (192 mg, 1.9 mmol) in DCM (10 mL) was added methanesulfonyl chloride (150 mg, 1.3 mmol) at 0° C. The resulting mixture was stirred at room temperature for 1 h. The mixture was concentrated under vacuum and used in next step directly without further purification. MS (ESI) m/z 626.2 [M+H]⁺.

### Step 3. 6-azido-2-(4-chloro-1-isopropyl-1H-pyrazol-5-yl)-4-(4-(1-ethyl-4-(trifluoromethyl)-1H-imidazol-2-yl) benzyl)-6,7-dihydropyrazolo[1,5-α]pyrimidin-5(4H)-one

A mixture of 2-(4-chloro-1-isopropyl-1H-pyrazol-5-yl)-4-(4-(1-ethyl-4-(trifluoromethyl)-1H-imidazol-2-yl)benzyl)-5-oxo-4,5,6,7-tetrahydropyrazolo[1,5-α]pyrimidin-6-yl methanesulfonate (401 mg, 0.64 mmol) and NaN₃ (83 mg, 1.3 mmol) in DMSO (5 mL) was stirred at room temperature for 1 h. The reaction mixture was diluted with water (20 mL) and extracted with EtOAc (50 mL). The organic phase was washed with brine, dried over anhydrous Na₂SO₄, concentrated under vacuum and purified by column chromatography on silica gel (eluting with 3/1 PE/EtOAc) to afford 6-azido-2-(4-chloro-1-isopropyl-1H-pyrazol-5-yl)-4-(4-(1-ethyl-4-(trifluoromethyl)-1H-imidazol-2-yl) benzyl)-6,7-dihydropyrazolo[1,5-α]pyrimidin-5(4H)-one (308 mg). MS (ESI) m/z 573.2 [M+H]⁺.

### Step 4. 6-amino-2-(4-chloro-1-isopropyl-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one

A mixture of 6-azido-2-(4-chloro-1-isopropyl-1H-pyrazol-5-yl)-4-(4-(1-ethyl-4-(trifluoromethyl)-1H-imidazol-2-yl) benzyl)-6,7-dihydropyrazolo[1,5-α]pyrimidin-5(4H)-one (300 mg, 0.52 mmol) and Pd/C (5%, 30 mg) in EtOAc (10 mL) under an atmosphere of H₂ was stirred at room temperature for 1 h. The mixture was filtered through celite, concentrated under vacuum and purified by on a Biotage Isolera One (C₁₈ column, eluting with 10% to 90% MeCN/H₂O) to afford Example 233 6-amino-2-(4-chloro-1-isopropyl-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one (26.2 mg). ¹H NMR (400 MHz, Methanol-d₄) δ 7.77 (s, 1H), 7.61-7.53 (m, 4H), 7.50 (s, 1H), 6.25 (s, 1H), 5.20-5.08 (m, 3H), 4.69 (d, J=5.8 Hz, 1H), 4.19 (d, J=7.9 Hz, 2H), 4.08 (q, J=7.3 Hz, 2H), 1.40 (d, J=6.6 Hz, 6H), 1.35 (t, J=7.3 Hz, 3H). MS (ESI) m/z 547.2 [M+H]⁺.

### Example 234. N-(2-(4-chloro-1-isopropyl-1H-pyrazol-5-yl)-4-(4-(1-ethyl-4-(trifluoromethyl)-1H-imidazol-2-yl)benzyl)-5-oxo-4,5,6,7-tetrahydropyrazolo[1,5-α]pyrimidin-6-yl)acetamide

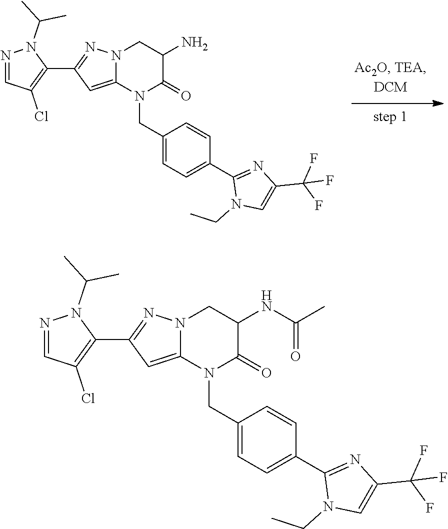 [SMILES: CCn1cc(C(F)(F)F)nc1-c1ccc(CN2C(=O)C(N)Cn3nc(-c4c(Cl)cnn4C(C)C)cc32)cc1 ; CCn1cc(C(F)(F)F)nc1-c1ccc(CN2C(=O)C(NC(C)=O)Cn3nc(-c4c(Cl)cnn4C(C)C)cc32)cc1]

### Step 1. N-(2-(4-chloro-1-isopropyl-1H-pyrazol-5-yl)-4-(4-(1-ethyl-4-(trifluoromethyl)-1H-imidazol-2-yl)benzyl)-5-oxo-4,5,6,7-tetrahydropyrazolo[1,5-α]pyrimidin-6-yl)acetamide

A mixture of 6-amino-2-(4-chloro-1-isopropyl-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one (70 mg, 0.13 mmol), acetyl chloride (10 mg, 0.13 mmol) and triethylamine (20 mg, 0.20 mmol) in DCM (2 mL) was stirred at room temperature for 30 min. The mixture was concentrated under vacuum and purified by on a Biotage Isolera One (C₁₈ column, eluting with 10% to 90% MeCN/H₂O) to afford Example 234 N-(2-(4-chloro-1-isopropyl-1H-pyrazol-5-yl)-4-(4-(1-ethyl-4-(trifluoromethyl)-1H-imidazol-2-yl)benzyl)-5-oxo-4,5,6,7-tetrahydropyrazolo[1,5-α]pyrimidin-6-yl)acetamide (25.1 mg). ¹H NMR (400 MHz, Methanol-d₄) δ 7.77 (s, 1H), 7.63-7.53 (m, 4H), 7.50 (s, 1H), 6.28 (s, 1H), 5.26-5.08 (m, 4H), 4.72 (dd, J=12.4, 7.4 Hz, 1H), 4.32 (t, J=12.2 Hz, 1H), 4.09 (q, J=7.3 Hz, 2H), 2.10 (s, 3H), 1.40 (d, J=6.6 Hz, 6H), 1.35 (t, J=7.3 Hz, 3H). MS (ESI) m/z 589.2 [M+H]⁺.

### Example 235. 2-(4-chloro-1-isopropyl-1H-pyrazol-5-yl)-6-(dimethylamino)-4-(4-(1-ethyl-4-(trifluoromethyl)-1H-imidazol-2-yl)benzyl)-6,7-dihydropyrazolo[1,5-α]pyrimidin-5(4H)-one

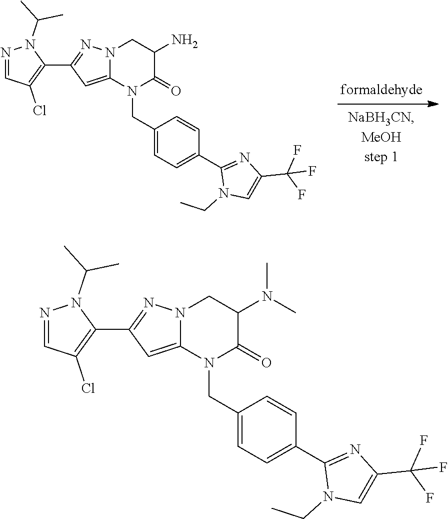 [SMILES: CCn1cc(C(F)(F)F)nc1-c1ccc(CN2C(=O)C(N)Cn3nc(-c4c(Cl)cnn4C(C)C)cc32)cc1 ; CCn1cc(C(F)(F)F)nc1-c1ccc(CN2C(=O)C(N(C)C)Cn3nc(-c4c(Cl)cnn4C(C)C)cc32)cc1]

### Step 1. 2-(4-chloro-1-isopropyl-1H-pyrazol-5-yl)-6-(dimethylamino)-4-(4-(1-ethyl-4-(trifluoromethyl)-1H-imidazol-2-yl)benzyl)-6,7-dihydropyrazolo[1,5-α]pyrimidin-5(4H)-one

To a solution of 6-amino-2-(4-chloro-1-isopropyl-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one (70 mg, 0.13 mmol) in MeOH (3 mL) was added formaldehyde (52 mg, 0.64 mmol). The solution was stirred for 30 min at room temperature then NaBH₃CN (17 mg, 0.26 mmol) was added. The resulting mixture was stirred for 30 min until TLC showed the starting material was consumed. The reaction mixture was quenched with aqueous HCl (2 M), extracted with DCM (10 mL×3). The combined organic phase was purified by prep-TLC (eluting with 1:1 EtOAc/PE) and Biotage Isolera One (C₁₈ column, eluting with 10% to 90% MeCN/H₂O) to afford Example 235 2-(4-chloro-1-isopropyl-1H-pyrazol-5-yl)-6-(dimethylamino)-4-(4-(1-ethyl-4-(trifluoromethyl)-1H-imidazol-2-yl)benzyl)-6,7-dihydropyrazolo[1,5-α]pyrimidin-5(4H)-one (13.7 mg). ¹H NMR (400 MHz, Methanol-d₄) δ 7.77 (s, 1H), 7.62-7.54 (m, 4H), 7.49 (s, 1H), 6.24 (s, 1H), 5.14 (d, J=10.7 Hz, 3H), 4.58 (dd, J=6.6, 3.5 Hz, 2H), 4.09 (q, J=7.3 Hz, 2H), 3.92 (t, J=6.8 Hz, 1H), 2.49 (s, 6H), 1.40 (dd, J=6.6, 2.3 Hz, 6H), 1.35 (t, J=7.3 Hz, 3H). MS (ESI) m/z 575.2 [M+H]⁺.

### Example 236. 2-(4-chloro-1-isopropyl-1H-pyrazol-5-yl)-4-(4-(1-ethyl-4-(trifluoromethyl)-1H-imidazol-2-yl)benzyl)-6-(methylamino)-6,7-dihydropyrazolo[1,5-α]pyrimidin-5(4H)-one

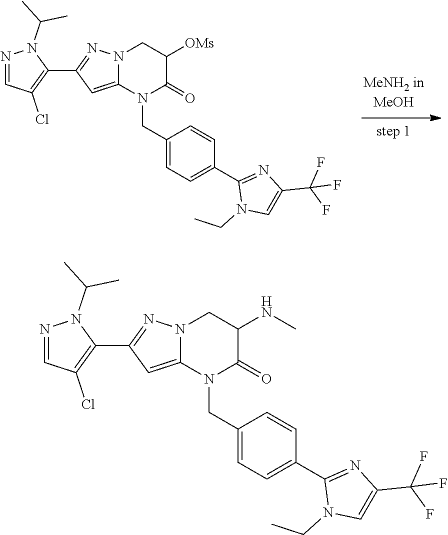 [SMILES: CCn1cc(C(F)(F)F)nc1-c1ccc(CN2C(=O)C(OS(C)(=O)=O)Cn3nc(-c4c(Cl)cnn4C(C)C)cc32)cc1 ; CCn1cc(C(F)(F)F)nc1-c1ccc(CN2C(=O)C(NC)Cn3nc(-c4c(Cl)cnn4C(C)C)cc32)cc1]

### Step 1. 2-(4-chloro-1-isopropyl-1H-pyrazol-5-yl)-4-(4-(1-ethyl-4-(trifluoromethyl)-1H-imidazol-2-yl)benzyl)-6-(methylamino)-6,7-dihydropyrazolo[1,5-α]pyrimidin-5(4H)-one

To a solution of 2-(4-chloro-1-isopropyl-1H-pyrazol-5-yl)-4-(4-(1-ethyl-4-(trifluoromethyl)-1H-imidazol-2-yl)benzyl)-5-oxo-4,5,6,7-tetrahydropyrazolo[1,5-α]pyrimidin-6-yl methanesulfonate (250 mg, 0.4 mmol) in DMSO (1 mL) was added methylamine in MeOH (30%, 1.5 mL, 0.80 mmol). The reaction was heated at 60° C. for 30 min. The mixture was diluted with water and extracted with EtOAc. The organic phase was washed with brine, dried over Na₂SO₄, concentrated under vacuum and purified by on a Biotage Isolera One (C₁₈ column, eluting with 10% to 90% MeCN/H₂O containing 0.1% formic acid) to afford Example 236 2-(4-chloro-1-isopropyl-1H-pyrazol-5-yl)-4-(4-(1-ethyl-4-(trifluoromethyl)-1H-imidazol-2-yl)benzyl)-6-(methylamino)-6,7-dihydropyrazolo[1,5-α]pyrimidin-5(4H)-one (25.1 mg). ¹H NMR (400 MHz, Methanol-d₄) δ 7.99-7.96 (m, 1H), 7.65 (s, 4H), 7.52 (s, 1H), 6.41 (s, 1H), 5.25 (d, J=4.0 Hz, 2H), 5.21-5.10 (m, 2H), 4.97 (dd, J=12.6, 7.3 Hz, 1H), 4.57 (t, J=12.2 Hz, 1H), 4.14 (q, J=7.3 Hz, 2H), 2.97 (s, 3H), 1.43-1.36 (m, 9H). MS (ESI) m/z 561.2 [M+H]⁺.

### Example 237. 2-(4-chloro-1-isopropyl-1H-pyrazol-5-yl)-N-(4-(1-ethyl-4-(trifluoromethyl)-1H-imidazol-2-yl)phenyl)-4,5,6,7-tetrahydropyrazolo[1,5-α]pyridin-4-amine

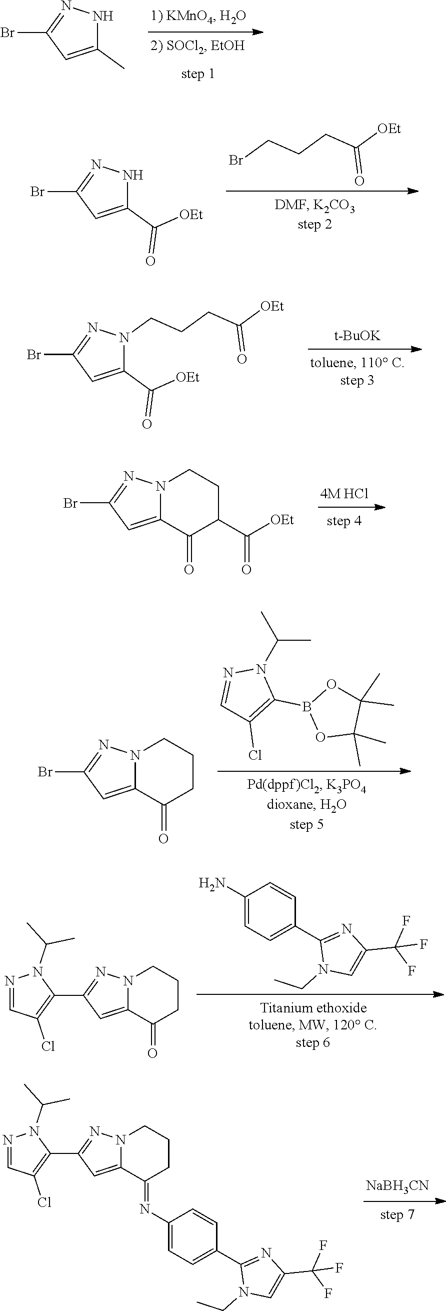

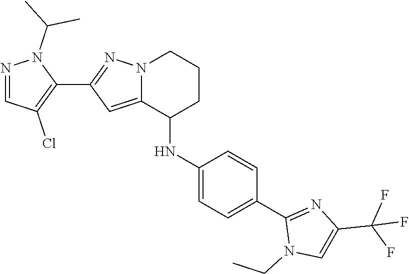 [SMILES: CCn1cc(C(F)(F)F)nc1-c1ccc(NC2CCCn3nc(-c4c(Cl)cnn4C(C)C)cc32)cc1]

### Step 1. ethyl 3-bromo-1H-pyrazole-5-carboxylate

To a solution of 3-bromo-5-methyl-1H-pyrazole (15.2 g, 95.02 mmol) in H₂O (250 mL) was added KMnO₄ (66.0 g, 0.42 mol) at 110° C. in 6 batches. The resulting solution was heated at 110° C. for 4.5 h. The mixture was filtered through celite while it was hot. The pH of filtrate was adjusted to 2 with 4 M aqueous HCl. The solution was extracted with EtOAc, dried over anhydrous Na₂SO₄ and concentrated to afford 3-bromo-1H-pyrazole-5-carboxylic acid (10.2 g) which used directly in the next step. To a solution of 3-bromo-1H-pyrazole-5-carboxylic acid (10.2 g, 53.68 mmol) in EtOH (80 mL) was added SOCl₂ (16 mL) at 0° C. The resulting solution was heated at reflux for 2 h. The solvent was removed under vacuum and the residue was washed with saturated aqueous NaHCO₃, dried over anhydrous Na₂SO₄ and concentrated to give ethyl 3-bromo-1H-pyrazole-5-carboxylate (12.3 g) MS (ESI) m/z 219.1, 221.1 [M+H]⁺.

### Step 2. ethyl 3-bromo-1-(4-ethoxy-4-oxobutyl)-1H-pyrazole-5-carboxylate

A mixture of ethyl 3-bromo-1H-pyrazole-5-carboxylate (5.91 g, 26.99 mmol), ethyl 4-bromobutanoate (5.26 g, 26.99 mmol) and K₂CO₃ (7.45 g, 53.98 mmol) in DMF (25 mL) was stirred at room temperature for 12 h. The mixture was filtered through celite and concentrated. The crude was purified on a Biotage Isolera One (C₁₈ column, eluting with 10% to 90% MeCN/H₂O) to afford ethyl 3-bromo-1-(4-ethoxy-4-oxobutyl)-1H-pyrazole-5-carboxylate (5.01 g). MS (ESI) m/z 333.2, 335.2[M+H]⁺.

### Step 3. ethyl 2-bromo-4-oxo-4,5,6,7-tetrahydropyrazolo[1,5-α]pyridine-5-carboxylate

A solution of ethyl 3-bromo-1-(4-ethoxy-4-oxobutyl)-1H-pyrazole-5-carboxylate (5.01 g, 15.0 mmol) and t-BuOK (3.38 g, 30.1 mmol) in toluene (80 mL) was stirred at 110° C. for 2 h. The mixture was filtered through celite and concentrated under vacuum to afford ethyl 2-bromo-4-oxo-4,5,6,7-tetrahydropyrazolo[1,5-α]pyridine-5-carboxylate (2.1 g) which was used directly in next step without further purification. MS (ESI) m/z 287.1, 289.1 [M+H]⁺.

### Step 4. 2-bromo-6,7-dihydropyrazolo[1,5-α]pyridin-4(5H)-one

A solution of ethyl 2-bromo-4-oxo-4,5,6,7-tetrahydropyrazolo[1,5-α]pyridine-5-carboxylate (2.1 g, 7.32 mmol) in aqueous HCl (6 M, 30 mL) was heated at 110° C. for 48 h. The reaction mixture was neutralized with NaOH (4 M), extracted with EtOAc, dried over anhydrous Na₂SO₄ and concentrated. The crude was purified on a Biotage Isolera One (C₁₈ column, eluting with 10% to 90% MeCN/H₂O) to afford 2-bromo-6,7-dihydropyrazolo[1,5-α]pyridin-4(5H)-one (960 mg). MS (ESI) m/z 215.0, 217.0[M+H]⁺.

### Step 5. 2-(4-chloro-1-isopropyl-1H-pyrazol-5-yl)-6,7-dihydropyrazolo[1,5-α]pyridin-4(5H)-one

2-(4-chloro-1-isopropyl-1H-pyrazol-5-yl)-6,7-dihydropyrazolo[1,5-α]pyridin-4(5H)-one was prepared from 2-bromo-6,7-dihydropyrazolo[1,5-α]pyridin-4(5H)-one and 4-chloro-1-isopropyl-5-(4,4,5,5-tetramethyl-1,3 ,2-dioxaborolan-2-yl)-1H-pyrazole according to step 3 of Example 1. MS (ESI) m/z 279.1 [M+H]⁺.

### Step 6. (E)-2-(4-chloro-1-isopropyl-1H-pyrazol-5-yl)-N-(4-(1-ethyl-4-(trifluoromethyl)-1H-imidazol-2-yl)phenyl)-6,7-dihydropyrazolo[1,5-α]pyridin-4(5H)-imine

A solution of 2-(4-chloro-1-isopropyl-1H-pyrazol-5-yl)-6,7-dihydropyrazolo[1,5-α]pyridin-4(5H)-one (480 mg, 1.73 mmol), 4-(1-ethyl-4-(trifluoromethyl)-1H-imidazol-2-yl)aniline (1.32 g, 5.18 mmol) and titanium ethoxide (789.6 mg, 3.46 mmol) in dry toluene (10 mL) was heated at 120° C. in microwave reactor for 4 h. Solvent was removed under vacuum and the residue was purified on a Biotage Isolera One (C₁₈ column, eluting with 10% to 90% MeCN/H₂O) to afford (E)-2-(4-chloro-1-isopropyl-1H-pyrazol-5-yl)-N-(4-(1-ethyl-4-(trifluoromethyl)-1H-imidazol-2-yl)phenyl)-6,7-dihydropyrazolo[1,5-α]pyridin-4(5H)-imine (280 mg). MS (ESI) m/z 516.2 [M+H]⁺.

### Step 7. 2-(4-chloro-1-isopropyl-1H-pyrazol-5-yl)-N-(4-(1-ethyl-4-(trifluoromethyl)-1H-imidazol-2-yl)phenyl)-4,5,6,7-tetrahydropyrazolo[1,5-α]pyridin-4-amine

To a solution of (E)-2-(4-chloro-1-isopropyl-1H-pyrazol-5-yl)-N-(4-(1-ethyl-4-(trifluoromethyl)-1H-imidazol-2-yl) phenyl)-6,7-dihydropyrazolo[1,5-α]pyridin-4(5H)-imine (150 mg, 0.29 mmol) in MeOH (5 mL) was added NaBH₃CN (91.75 mg, 1.46 mmol). The resulting mixture was stirred at room temperature for 2 h. The reaction was quenched with water, extracted with EtOAc, dried over anhydrous Na₂SO₄, concentrated and purified by prep-HPLC (condition 2) to afford Example 237 2-(4-chloro-1-isopropyl-1H-pyrazol-5-yl)-N-(4-(1-ethyl-4-(trifluoromethyl)-1H-imidazol-2-yl)phenyl)-4,5,6,7-tetrahydropyrazolo[1,5-α]pyridin-4-amine (132 mg).

Table 17. The compounds listed in Table 17 were synthesized according to Example 237 using the appropriate commercially available reagents and/or intermediates described above. Enantiomers, when generated, were separated by chiral HPLC and absolute stereochemistries were arbitrarily assigned.

TABLE 17

| Example | | | |
|---|---|---|---|
| number | Structure | ¹H NMR (δ ppm) | MS (ESI) |
| 237 | 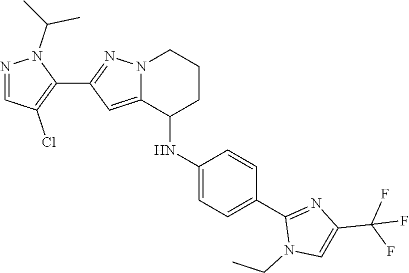 [SMILES: CCn1cc(C(F)(F)F)nc1-c1ccc(NC2CCCn3nc(-c4c(Cl)cnn4C(C)C)cc32)cc1] 2-(4-chloro-1-isopropyl-1H- pyrazol-5-yl)-N-(4-(1-ethyl-4- (trifluoromethyl)-1H- imidazol-2-yl)phenyl)-4,5,6,7- tetrahydropyrazolo[1,5- a]pyridin-4-amine | ¹H NMR (400 MHz, Chloroform-d) δ 7.49 (s, 1H), 7.44 (d, J = 8.3 Hz, 2H), 7.32 (s, 1H), 6.77 (d, J = 8.3 Hz, 2H), 6.62 (s, 1H), 5.21-5.15 (m, 1H), 4.87 (s, 1H), 4.34- 4.18 (m, 2H), 4.06 (q, J = 7.3 Hz, 2H), 2.41-2.24 (m, 2H), 2.15 (s, 1H), 1.87 (q, J = 10.6, 1H), 1.46 (d, J = 6.6 Hz, 6H), 1.43 (d, J = 7.2 Hz, 3H). | m/z: 518.2 [M + H]⁺ |
| 238 | 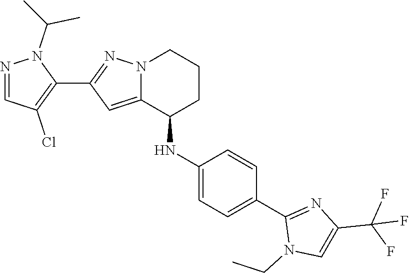 [SMILES: CCn1cc(C(F)(F)F)nc1-c1ccc(N[C@@H]2CCCn3nc(-c4c(Cl)cnn4C(C)C)cc32)cc1] (R)-2-(4-chloro-1-isopropyl- 1H-pyrazol-5-yl)-N-(4-(1- ethyl-4-(trifluoromethyl)-1H- imidazol-2-yl)phenyl)-4,5,6,7- tetrahydropyrazolo[1,5- a]pyridin-4-amine | ¹H NMR (400 MHz, Chloroform-d) δ 7.48 (s, 1H), 7.43 (d, J = 8.5 Hz, 2H), 7.34- 7.29 (m, 1H), 6.76 (d, J = 8.6 Hz, 2H), 6.61 (s, 1H), 5.21- 5.14 (m, 1H), 4.88-4.83 (m, 1H), 4.33-4.17 (m, 3H), 4.05 (q, J = 7.3 Hz, 2H), 2.39-2.23 (m, 2H), 2.18-2.07 (m, 1H), 1.92-1.81 (m, 1H), 1.46-1.41 (m, 9H). | m/z: 518.2 [M + H]⁺ |
| 239 | 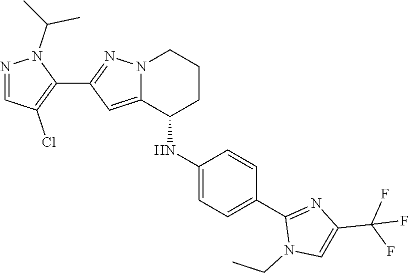 [SMILES: CCn1cc(C(F)(F)F)nc1-c1ccc(N[C@H]2CCCn3nc(-c4c(Cl)cnn4C(C)C)cc32)cc1] (S)-2-(4-chloro-1-isopropyl- 1H-pyrazol-5-yl)-N-(4-(1- ethyl-4-(trifluoromethyl)-1H- imidazol-2-yl)phenyl)-4,5,6,7- tetrahydropyrazolo[1,5- a]pyridin-4-amine | ¹H NMR (400 MHz, Chloroform-d) δ 7.48 (s, 1H), 7.43 (d, J = 8.6 Hz, 2H), 7.33- 7.30 (m, 1H), 6.76 (d, J = 8.6 Hz, 2H), 6.61 (s, 1H), 5.25- 5.11 (m, 1H), 4.90-4.82 (m, 1H), 4.34-4.15 (m, 3H), 4.05 (q, J = 7.3 Hz, 2H), 2.41-2.22 (m, 2H), 2.18- 2.06 (m, 1H), 1.91-1.80 (m, 1H), 1.46-1.41 (m, 9H). | m/z: 518.2 [M + H]⁺ |
| 240 | 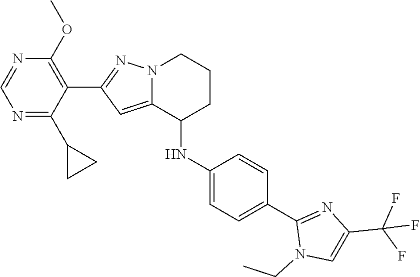 [SMILES: CCn1cc(C(F)(F)F)nc1-c1ccc(NC2CCCn3nc(-c4c(OC)ncnc4C4CC4)cc32)cc1] 2-(4-cyclopropyl-6- methoxypyrimidin-5-yl)-N-(4- (1-ethyl-4-(trifluoromethyl)- 1H-imidazol-2-yl)phenyl)- 4,5,6,7- tetrahydropyrazolo[1,5- a]pyridin-4-amine | ¹H NMR (400 MHz, Chloroform-d) δ 8.56 (s, 1H), 7.44 (d, J = 8.7 Hz, 2H), 7.32 (d, J = 1.2 Hz, 1H), 6.77 (d, J = 8.7 Hz, 2H), 6.37 (s, 1H), 4.88 (s, 1H), 4.38-4.16 (m, 3H), 4.07 (s, 2H), 3.93 (s, 3H), 2.40-2.14 (m, 4H), 1.88 (d, J = 10.4 Hz, 1H), 1.44 (s, 3H), 1.18 (s, 2H), 0.97-0.90 (m, 2H). | m/z: 524.2 [M + H]⁺ |
| 241 | 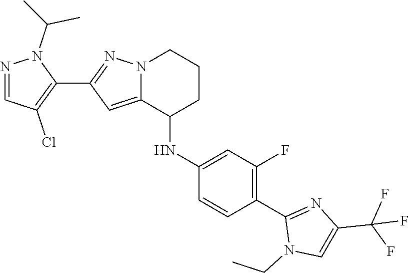 [SMILES: CCn1cc(C(F)(F)F)nc1-c1ccc(NC2CCCn3nc(-c4c(Cl)cnn4C(C)C)cc32)cc1F] 2-(4-chloro-1-isopropyl-1H- pyrazol-5-yl)-N-(4-(1-ethyl-4- (trifluoromethyl)-1H- imidazol-2-yl)-3- fluorophenyl)-4,5,6,7- tetrahydropyrazolo[1,5- a]pyridin-4-amine | ¹H NMR (400 MHz, Chloroform-d) δ 7.49 (s, 1H), 7.40-7.34 (m, 2H), 6.63 (s, 1H), 6.56 (dd, J = 8.5, 2.3 Hz, 1H), 6.46 (dd, J = 12.4, 2.2 Hz, 1H), 5.18 (m, 1H), 4.83 (s, 1H), 4.37-4.18 (m, 3H), 3.93 (q, J = 7.2 Hz, 2H), 2.36- 2.19 (m, 2H), 2.06-1.82 (m, 2H), 1.46 (d, J = 6.6 Hz, 6H), 1.40 (t, J = 7.3 Hz, 3H). | m/z: 536.2 [M + H]⁺ |
| 242 | 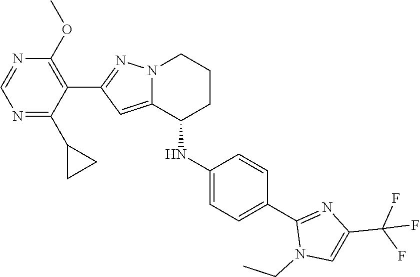 [SMILES: CCn1cc(C(F)(F)F)nc1-c1ccc(N[C@H]2CCCn3nc(-c4c(OC)ncnc4C4CC4)cc32)cc1] (S)-2-(4-cyclopropyl-6- methoxypyrimidin-5-yl)-N-(4- (1-ethyl-4-(trifluoromethyl)- 1H-imidazol-2-yl)phenyl)- 4,5,6,7- tetrahydropyrazolo[1,5- a]pyridin-4-amine | ¹H NMR (400 MHz, Chloroform-d) δ 8.56 (s, 1H), 7.44 (d, J = 8.6 Hz, 2H), 7.32 (d, J = 1.1 Hz, 1H), 6.77 (d, J = 8.7 Hz, 2H), 6.37 (s, 1H), 4.88 (s, 1H), 4.36-4.17 (m, 3H), 4.06 (d, J = 7.3 Hz, 2H), 3.93 (s, 3H), 2.27 (dd, J = 8.6, 4.0 Hz, 4H), 1.90 (d, J = 8.1 Hz, 1H), 1.44 (t, J = 7.3 Hz, 3H), 1.17 (d, J = 3.8 Hz, 2H), 0.93 (t, J = 7.2 Hz, 2H). | m/z: 524.2 [M + H]⁺ |
| 243 | 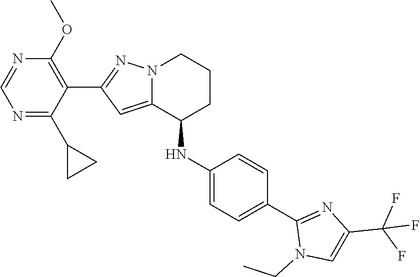 [SMILES: CCn1cc(C(F)(F)F)nc1-c1ccc(N[C@@H]2CCCn3nc(-c4c(OC)ncnc4C4CC4)cc32)cc1] (R)-2-(4-cyclopropyl-6- methoxypyrimidin-5-yl)-N-(4- (1-ethyl-4-(trifluoromethyl)- 1H-imidazol-2-yl)phenyl)- 4,5,6,7- tetrahydropyrazolo[1,5- a]pyridin-4-amine | ¹H NMR (400 MHz, Chloroform-d) δ 8.56 (s, 1H), 7.44 (d, J = 8.7 Hz, 2H), 7.32 (d, J = 1.2 Hz, 1H), 6.77 (d, J = 8.7 Hz, 2H), 6.37 (s, 1H), 4.88 (s, 1H), 4.38-4.16 (m, 3H), 4.07 (s, 2H), 3.93 (s, 3H), 2.40-2.14 (m, 4H), 1.88 (d, J = 10.4 Hz, 1H), 1.44 (s, 3H), 1.18 (s, 2H), 0.97-0.90 (m, 2H). | m/z: 524.2 [M + H]⁺ |
| 244 | 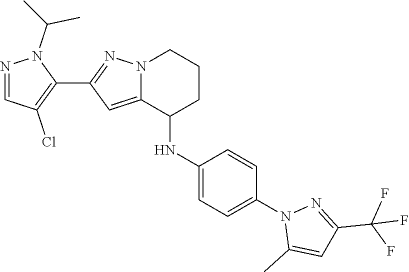 [SMILES: Cc1cc(C(F)(F)F)nn1-c1ccc(NC2CCCn3nc(-c4c(Cl)cnn4C(C)C)cc32)cc1] 2-(4-chloro-1-isopropyl-1H- pyrazol-5-yl)-N-(4-(5-methyl- 3-(trifluoromethyl)-1H- pyrazol-1-yl)phenyl)-4,5,6,7- tetrahydropyrazolo[1,5- a]pyridin-4-amine | ¹H NMR (400 MHz, Chloroform-d) δ 7.48 (s, 1H), 7.26 (d, J = 1.6 Hz, 1H), 7.24 (s, 1H), 6.76 (d, J = 8.8 Hz, 2H), 6.63-6.60 (m, 1H), 6.41 (s, 1H), 5.18 (m, 1H), 4.84 (dd, J = 8.1, 4.7 Hz, 1H), 4.33-4.17 (m, 2H), 2.39-2.25 (m, 5H), 2.17-2.06 (m, 1H), 1.91-1.81 (m, 1H), 1.46 (d, J = 6.6 Hz, 6H). | m/z: 504.2 [M + H]⁺ |
| 245 | 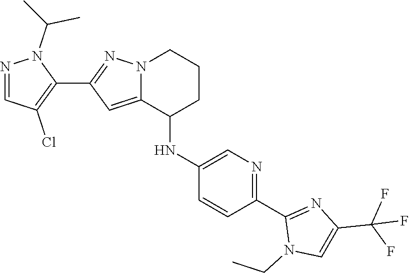 [SMILES: CCn1cc(C(F)(F)F)nc1-c1ccc(NC2CCCn3nc(-c4c(Cl)cnn4C(C)C)cc32)cn1] 2-(4-chloro-1-isopropyl-1H- pyrazol-5-yl)-N-(6-(1-ethyl-4- (trifluoromethyl)-1H- imidazol-2-yl)pyridin-3-yl)- 4,5,6,7- tetrahydropyrazolo[1,5- a]pyridin-4-amine | ¹H NMR (400 MHz, Chloroform-d) δ 8.11-8.01 (m, 2H), 7.48 (s, 1H), 7.29 (s, IH), 7.10-7.04 (m, 1H), 6.62 (s, 1H), 5.22-5.14 (m, 1H), 4.87 (s, 1H), 4.59 (q, J = 7.1 Hz, 2H), 4.34-4.18 (m, 3H), 2.40-2.26 (m, 2H), 2.15 (dd, J = 8.2, 5.3 Hz, 1H), 1.93-1.84 (m, 1H), 1.45 (dd, J = 6.9, 3.2 Hz, 9H). | m/z: 519.2 [M + H]⁺ |
| 246 | 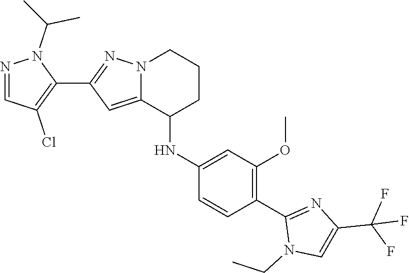 [SMILES: CCn1cc(C(F)(F)F)nc1-c1ccc(NC2CCCn3nc(-c4c(Cl)cnn4C(C)C)cc32)cc1OC] 2-(4-chloro-1-isopropyl-1H- pyrazol-5-yl)-N-(4-(1-ethyl-4- (trifluoromethyl)-1H- imidazol-2-yl)-3- methoxyphenyl)-4,5,6,7- tetrahydropyrazolo[1,5- a]pyridin-4-amine | ¹H NMR (400 MHz, Chloroform-d) δ 7.46 (s, 1H), 7.30 (s, 1H), 7.24 (s, 1H), 6.61 (s, 1H), 6.35 (d, J = 8.3 Hz, 1H), 6.23 (s, 1H), 5.24- 5.09 (m, 1H), 4.82 (d, J = 5.1 Hz, 1H), 4.26 (d, J = 5.3 Hz, 1H), 4.20 (dd, J = 8.6, 4.5 Hz, 1H), 3.81 (q, J = 7.3 Hz, 2H), 3.72 (s, 3H), 2.30 (t, J = 14.3 Hz, 2H), 2.19-2.06 (m, 1H), 1.85 (q, J = 10.2 Hz, 1H), 1.43 (dd, J = 6.5, 2.3 Hz, 6H), 1.32 (t, J = 7.3 Hz, 3H). | m/z: 548.2 [M + H]⁺ |
| 247 | 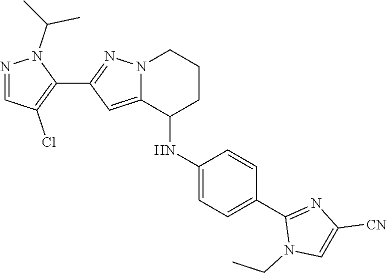 [SMILES: CCn1cc(C#N)nc1-c1ccc(NC2CCCn3nc(-c4c(Cl)cnn4C(C)C)cc32)cc1] 2-(4-((2-(4-chloro-1- isopropyl-1H-pyrazol-5-yl)- 4,5,6,7- tetrahydropyrazolo[1,5- a]pyridin-4-yl)amino)phenyl)- 1-ethyl-1H-imidazole-4- carbonitrile | ¹H NMR (400 MHz, Chloroform-d) δ 7.53 (s, 1H), 7.47 (s, 1H), 7.41 (d, J = 8.3 Hz, 2H), 6.82-6.73 (m, 2H), 6.61 (s, 1H), 5.18 (m, 1H), 4.86 (s, 1H), 4.35-4.17 (m, 3H), 4.07 (q, J = 7.3 Hz, 2H), 2.32 (s, 2H), 2.20-2.07 (m, 1H), 1.88 (m, 1H), 1.43 (dd, J = 12.9, 6.9 Hz, 9H). | m/z: 475.2 [M + H]⁺ |
| 248 | 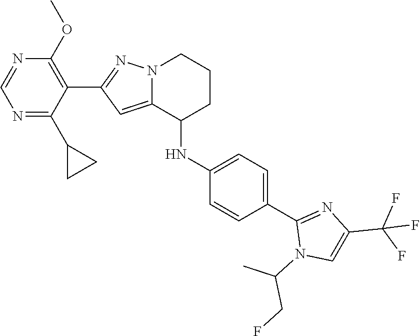 [SMILES: COc1ncnc(C2CC2)c1-c1cc2n(n1)CCCC2Nc1ccc(-c2nc(C(F)(F)F)cn2C(C)CF)cc1] 2-(4-cyclopropyl-6- methoxypyrimidin-5-yl)-N-(4- (1-(1-fluoropropan-2-yl)-4- (trifluoromethyl)-1H- imidazol-2-yl)phenyl)-4,5,6,7- tetrahydropyrazolo[1,5- a]pyridin-4-amine | ¹H NMR (400 MHz, Methanol-d₄) δ 8.49 (s, 1H), 7.87 (s, 1H), 7.34 (d, J = 8.6 Hz, 2H), 6.88 (d, J = 8.6 Hz, 2H), 6.29 (s, 1H), 4.94 (d, J = 3.9 Hz, 1H), 4.76 (d, J = 6.4 Hz, 1H), 4.67 (d, J = 6.0 Hz, 1H), 4.55 (d, J = 6.6 Hz, 1H), 4.28-4.14 (m, 2H), 3.88 (s, 3H), 2.30 (d, J = 5.3 Hz, 2H), 2.20-2.09 (m, 2H), 1.90 (t, J = 9.2 Hz, 1H), 1.45 (d, J = 6.9 Hz, 3H), 1.10 (dd, J = 4.6, 3.1 Hz, 2H), 0.91 (dd, J = 7.6, 3.7 Hz, 2H). | m/z: 556.1 [M + H]⁺ |
| 249 | 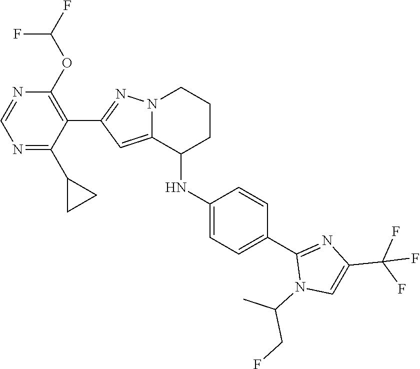 [SMILES: CC(CF)n1cc(C(F)(F)F)nc1-c1ccc(NC2CCCn3nc(-c4c(OC(F)F)ncnc4C4CC4)cc32)cc1] 2-(4-cyclopropyl-6- (difluoromethoxy)pyrimidin- 5-yl)-N-(4-(1-(1- fluoropropan-2-yl)-4- (trifluoromethyl)-1H- imidazol-2-yl)phenyl)-4,5,6,7- tetrahydropyrazolo[1,5- a]pyridin-4-amine | ¹H NMR (400 MHz, Chloroform-d) δ 8.55 (s, 1H), 7.48 (t, J = 72.0 Hz, 1H), 7.44 (s, 1H), 7.38 (d, J = 8.3 Hz, 2H), 6.77 (d, J = 8.4 Hz, 2H), 6.40 (s, 1H), 4.87 (dd, J = 7.8, 4.7 Hz, 1H), 4.75-4.64 (m, 1H), 4.59 (d, J = 4.0 Hz, 1H), 4.47 (d, J = 3.9 Hz, 1H), 4.34- 4.18 (m, 2H), 2.45 (tt, J = 8.4, 4.8 Hz, 1H), 2.39-2.26 (m, 2H), 2.15 (d, J = 9.1 Hz, 1H), 1.88 (q, J = 10.5, 9.6 Hz, 1H), 1.50 (d, J = 6.9 Hz, 3H), 1.22 (q, J = 3.4 Hz, 2H), 1.04-0.98 (m, 2H). | m/z: 592.2 [M + H]⁺ |
| 250 | 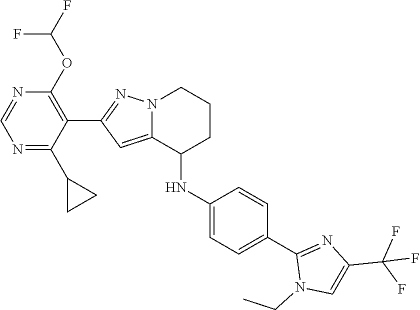 [SMILES: CCn1cc(C(F)(F)F)nc1-c1ccc(NC2CCCn3nc(-c4c(OC(F)F)ncnc4C4CC4)cc32)cc1] 2-(4-cyclopropyl-6- (difluoromethoxy)pyrimidin- 5-yl)-N-(4-(1-ethyl-4- (trifluoromethyl)-1H- imidazol-2-yl)phenyl)-4,5,6,7- tetrahydropyrazolo[1,5- a]pyridin-4-amine | ¹H NMR (400 MHz, Chloroform-d) δ 8.55 (s, 1H), 7.48 (t, J = 71.6 Hz, 1H), 7.44 (d, J = 7.9 Hz, 2H), 7.32 (s, 1H), 6.78 (d, J = 8.0 Hz, 2H), 6.40 (s, 1H), 4.88 (s, 1H), 4.34-4.18 (m, 2H), 4.06 (q, J = 7.1 Hz, 2H), 2.44 (dt, J = 7.9, 3.7 Hz, 1H), 2.39-2.27 (m, 2H), 2.16 (s, 1H), 1.88 (q, J = 9.7 Hz, 1H), 1.44 (t, J = 7.1 Hz, 3H), 1.24-1.19 (m, 2H), 1.04-0.98 (m, 2H). | m/z: 560.2 [M + H]⁺ |
| 251 | 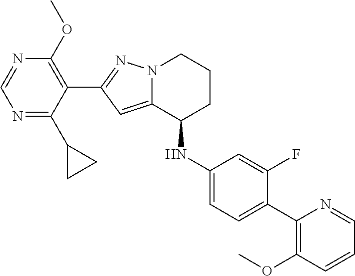 [SMILES: COc1cccnc1-c1ccc(N[C@@H]2CCCn3nc(-c4c(OC)ncnc4C4CC4)cc32)cc1F] (R)-2-(4-cyclopropyl-6- methoxypyrimidin-5-yl)-N-(3- fluoro-4-(3-methoxypyridin- 2-yl)phenyl)-4,5,6,7- tetrahydropyrazolo[1,5- a]pyridin-4-amine | ¹H NMR (400 MHz, Chloroform-d) δ 8.55 (s, 1H), 8.29 (dd, J = 4.3, 1.7 Hz, 1H), 7.38 (t, J = 8.3 Hz, 1H), 7.30- 7.24 (m, 2H), 6.56 (dd, J = 8.4, 2.3 Hz, 1H), 6.46 (dd, J = 12.5, 2.2 Hz, 1H), 6.39 (s, 1H), 4.85-4.79 (m, 1H), 4.34- 4.17 (m, 3H), 3.93 (s, 3H), 3.85 (s, 3H), 2.37-2.24 (m, 3H), 2.16-2.08 (m, 1H), 1.92- 1.82 (m, 1H), 1.17 (t, J = 4.2 Hz, 2H), 0.96-0.91 (m, 2H). | m/z: 487.2 [M + H]⁺ |
| 252 | 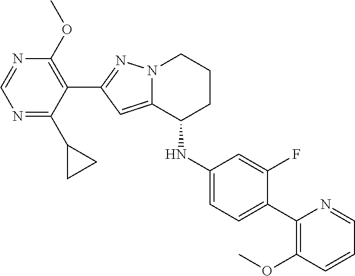 [SMILES: COc1cccnc1-c1ccc(N[C@H]2CCCn3nc(-c4c(OC)ncnc4C4CC4)cc32)cc1F] (S)-2-(4-cyclopropyl-6- methoxypyrimidin-5-yl)-N-(3- fluoro-4-(3-methoxypyridin- 2-yl)phenyl)-4,5,6,7- tetrahydropyrazolo[1,5- a]pyridin-4-amine | ¹H NMR (400 MHz, Chloroform-d) δ 8.55 (s, 1H), 8.29 (dd, J = 4.3, 1.7 Hz, 1H), 7.38 (t, J = 8.3 Hz, 1H), 7.30- 7.24 (m, 2H), 6.56 (dd, J = 8.4, 2.3 Hz, 1H), 6.46 (dd, J = 12.5, 2.2 Hz, 1H), 6.39 (s, 1H), 4.85-4.79 (m, 1H), 4.33- 4.18 (m, 3H), 3.93 (s, 3H), 3.85 (s, 3H), 2.37-2.24 (m, 3H), 2.16-2.08 (m, 1H), 1.92- 1.82 (m, 1H), 1.17 (t, J = 4.2 Hz, 2H), 0.96-0.91 (m, 2H). | m/z: 487.2 [M + H]⁺ |
| 253 | 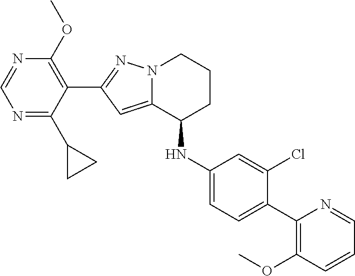 [SMILES: COc1cccnc1-c1ccc(N[C@@H]2CCCn3nc(-c4c(OC)ncnc4C4CC4)cc32)cc1Cl] (R)-N-(3-chloro-4-(3- methoxypyridin-2-yl)phenyl)- 2-(4-cyclopropyl-6- methoxypyrimidin-5-yl)- 4,5,6,7- tetrahydropyrazolo[1,5- a]pyridin-4-amine | ¹H NMR (400 MHz, Chloroform-d) δ 8.54 (s, 1H), 8.27 (t, J = 2.9 Hz, 1H), 7.29 (d, J = 2.9 Hz, 2H), 7.25-7.23 (m, 1H), 6.77 (d, J = 2.2 Hz, 1H), 6.65 (dd, J = 8.4, 2.2 Hz, 1H), 6.36 (s, 1H), 4.80 (s, 1H), 4.30-4.16 (m, 3H), 3.92 (s, 3H), 3.82 (s, 3H), 2.26 (ddd, J = 12.6, 8.3, 4.6 Hz, 3H), 2.14-2.05 (m, 1H), 1.91- 1.81 (m, 1H), 1.16 (t, J = 4.4 Hz, 2H), 0.94-0.89 (m, 2H). | m/z: 503.2 [M + H]⁺ |
| 254 | 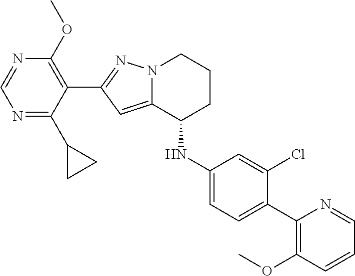 [SMILES: COc1cccnc1-c1ccc(N[C@H]2CCCn3nc(-c4c(OC)ncnc4C4CC4)cc32)cc1Cl] (S)-N-(3-chloro-4-(3- methoxypyridin-2-yl)phenyl)- 2-(4-cyclopropyl-6- methoxypyrimidin-5-yl)- 4,5,6,7- tetrahydropyrazolo[1,5- a]pyridin-4-amine | ¹H NMR (400 MHz, Chloroform-d) δ 8.54 (s, 1H), 8.27 (t, J = 2.9 Hz, 1H), 7.29 (d, J = 2.9 Hz, 2H), 7.25-7.22 (m, 1H), 6.77 (d, J = 2.2 Hz, 1H), 6.65 (dd, J = 8.4, 2.2 Hz, 1H), 6.36 (s, 1H), 4.80 (s, 1H), 4.30-4.16 (m, 3H), 3.92 (s, 3H), 3.82 (s, 3H), 2.26 (m, 3H), 2.14-2.05 (m, 1H), 1.91- 1.82 (m, 1H), 1.16 (t, J = 4.4 Hz, 2H), 0.95-0.89 (m, 2H). | m/z: 503.2 [M + H]⁺ |
| 255 | 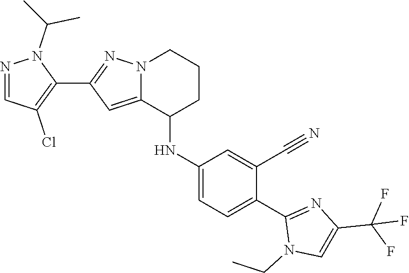 [SMILES: CCn1cc(C(F)(F)F)nc1-c1ccc(NC2CCCn3nc(-c4c(Cl)cnn4C(C)C)cc32)cc1C#N] 5-((2-(4-chloro-1-isopropyl- 1H-pyrazol-5-yl)-4,5,6,7- tetrahydropyrazolo[1,5- a]pyridin-4-yl)amino)-2-(1- ethyl-4-(trifluoromethyl)-1H- imidazol-2-yl)benzonitrile | ¹H NMR (400 MHz, Chloroform-d) δ 7.51 (s, 1H), 7.43 (d, J = 8.1 Hz, 2H), 7.01 (d, J = 2.1 Hz, 1H), 6.95 (d, J = 8.5 Hz, 1H), 6.62 (s, 1H), 5.20 (m, 1H), 4.85 (d, J = 5.1 Hz, 1H), 4.36-4.20 (m, 2H), 3.99 (q, J = 7.2 Hz, 2H), 2.43- 2.27 (m, 2H), 2.17 (s, 1H), 1.95-1.84 (m, 1H), 1.47 (d, J = 6.6 Hz, 6H), 1.42 (t, J = 7.3 Hz, 3H). | m/z: 543.3 [M + H]⁺ |

### Example 256. 2-(4-chloro-1-isopropyl-1H-pyrazol-5-yl)-N-(4-(1-ethyl-4-(trifluoromethyl)-1H-imidazol-2-yl)phenyl)-5,6,7,8-tetrahydro-[1,2,4]triazolo[1,5-α]pyridin-8-amine

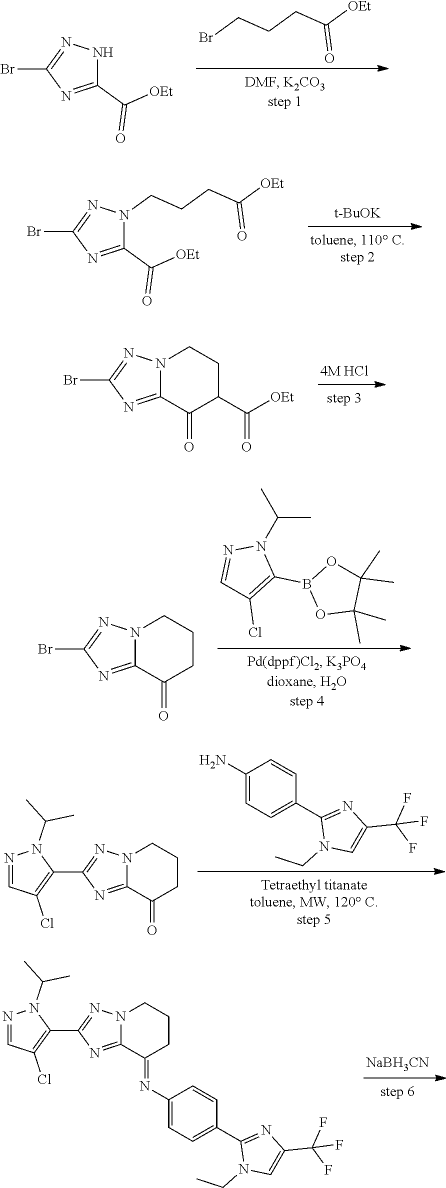

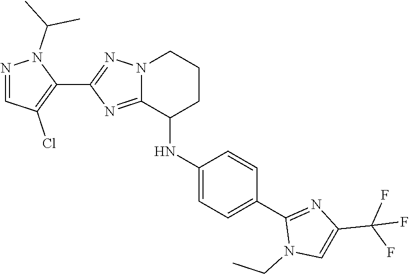 [SMILES: CCn1cc(C(F)(F)F)nc1-c1ccc(NC2CCCn3nc(-c4c(Cl)cnn4C(C)C)nc32)cc1]

Example 256 2-(4-chloro-1-isopropyl-1H-pyrazol-5-yl)-N-(4-(1-ethyl-4-(trifluoromethyl)-1H-imidazol-2-yl)phenyl)-5,6,7,8-tetrahydro-[1,2,4]triazolo[1,5-α]pyridin-8-amine was prepared from ethyl 3-bromo-1H-1,2,4-triazole-5-carboxylate following step 2 to step 7 of Example 237.

Table 18. The compounds listed in Table 18 were synthesized according to Example 256 using the appropriate commercially available reagents and/or intermediates described above.

TABLE 18

| Example | | | |
|---|---|---|---|
| number | Structure | ¹H NMR (δ ppm) | MS (ESI) |
| 256 | 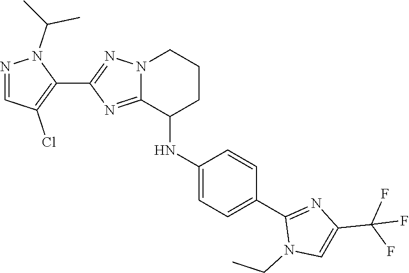 [SMILES: CCn1cc(C(F)(F)F)nc1-c1ccc(NC2CCCn3nc(-c4c(Cl)cnn4C(C)C)nc32)cc1] 2-(4-chloro-1-isopropyl-1H- pyrazol-5-yl)-N-(4-(1-ethyl-4- (trifluoromethyl)-1H- imidazol-2-yl)phenyl)-5,6,7,8- tetrahydro-[1,2,4]triazolo[1,5- a]pyridin-8-amine | ¹H NMR (400 MHz, Chloroform-d) δ 7.52 (s, 1H), 7.43 (d, J = 8.1 Hz, 2H), 7.32 (s, 1H), 6.84 (d, J = 8.2 Hz, 2H), 5.24-5.11 (m, 1H), 4.89- 4.80 (m, 1H), 4.36-4.29 (m, 2H), 4.04 (q, J = 7.2 Hz, 2H), 2.50 (d, J = 6.1 Hz, 1H), 2.35 (s, 1H), 2.27-2.14 (m, 1H), 2.01 (t, J = 9.4 Hz, 1H), 1.48- 1.40 (m, 9H). | m/z: 519.2 [M + H]⁺ |
| 257 | 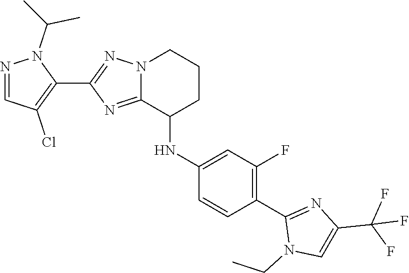 [SMILES: CCn1cc(C(F)(F)F)nc1-c1ccc(NC2CCCn3nc(-c4c(Cl)cnn4C(C)C)nc32)cc1F] 2-(4-chloro-1-isopropyl-1H- pyrazol-5-yl)-N-(4-(1-ethyl-4- (trifluoromethyl)-1H- imidazol-2-yl)-3- fluorophenyl)-5,6,7,8- tetrahydro-[1,2,4]triazolo[1,5- a]pyridin-8-amine | ¹H NMR (400 MHz, Chloroform-d) δ 7.53 (s, 1H), 7.36 (s, 2H), 6.61 (d, J = 10.8 Hz, 2H), 5.25-5.13 (m, 1H), 4.87-4.66 (m, 2H), 4.33 (s, 2H), 3.90 (q, J = 12 Hz, 2H), 2.53 (s, 1H), 2.36 (s, 1H), 2.21 (s, 1H), 2.02 (s, 1H), 1.45 (dd, J = 14.1, 6.5 Hz, 6H), 1.38 (t, J = 1.3 Hz, 3H). | m/z: 537.2 [M + H]⁺ |

### Example 258. 2-(4-chloro-1-isopropyl-1H-pyrazol-5-yl)-N-(4-(1-ethyl-4-(trifluoromethyl)-1H-imidazol-2-yl)phenyl)-N-methyl-4,5,6,7-tetrahydropyrazolo[1,5-α]pyridin-4-amine

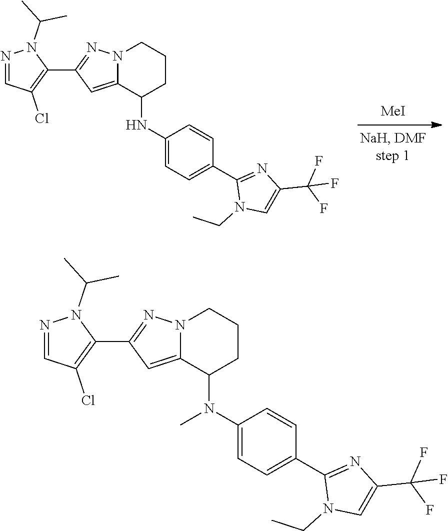 [SMILES: "MeI" ; CCn1cc(C(F)(F)F)nc1-c1ccc(NC2CCCn3nc(-c4c(Cl)cnn4C(C)C)cc32)cc1 ; CCn1cc(C(F)(F)F)nc1-c1ccc(N(C)C2CCCn3nc(-c4c(Cl)cnn4C(C)C)cc32)cc1]

### Step 1. 2-(4-chloro-1-isopropyl-1H-pyrazol-5-yl)-N-(4-(1-ethyl-4-(trifluoromethyl)-1H-imidazol-2-yl)phenyl)-N-methyl-4,5,6,7-tetrahydropyrazolo[1,5-α]pyridin-4-amine

To the solution of 2-(4-chloro-1-isopropyl-1H-pyrazol-5-yl)-N-(4-(1-ethyl-4-(trifluoromethyl)-1H-imidazol-2-yl) phenyl)-4,5,6,7-tetrahydropyrazolo[1,5-α]pyridin-4-amine (prepared according to Example 237, 30 mg, 0.058 mmol) in dry DMF (2 mL) was added NaH (1.53 mg, 60% in mineral oil, 0.064 mmol) at 0° C. The mixture was stirred at room temperature for 20 min then iodomethane (8.24 mg, 0.058 mmol) was added. The resulting mixture was stirred at room temperature for 1 h then quenched with water concentrated and purified by prep-HPLC (condition 3) to give Example 258 2-(4-chloro-1-isopropyl-1H-pyrazol-5-yl)-N-(4-(1-ethyl-4-(trifluoromethyl)-1H-imidazol-2-yl)phenyl)-N-methyl-4,5,6,7-tetrahydropyrazolo[1,5-α]pyridin-4-amine (11.5 mg).

Table 19. The compounds listed in Table 19 were synthesized according to Example 258 using the appropriate commercially available reagents and/or intermediates described above.

TABLE 19

| Example | | | |
|---|---|---|---|
| number | Stucture | ¹H NMR (δ ppm) | MS (ESI) |
| 258 | 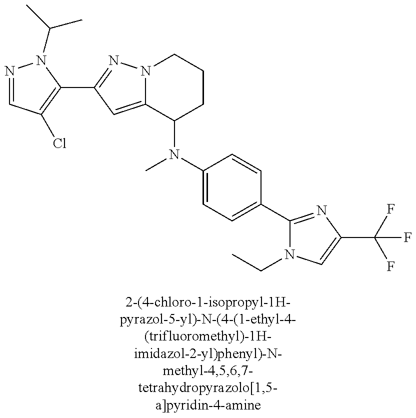 [SMILES: CCn1cc(C(F)(F)F)nc1-c1ccc(N(C)C2CCCn3nc(-c4c(Cl)cnn4C(C)C)cc32)cc1] | ¹H NMR (400 MHz, Chloroform-d) δ 7.53-7.46 (m, 3H), 7.33 (s, 1H), 6.93 (d, J = 8.4 Hz, 2H), 6.48 (s, 1H), 5.24-5.17 (m, 2H), 4.36 (d, J = 13.1 Hz, 1H), 4.20-4.13 (m, 1H), 4.08 (q, J = 7.3 Hz, 2H), 2.82 (s, 3H), 2.30 (d, J = 13.7 Hz, 1H), 2.26-2.10 (m, 2H), 2.01-1.93 (m, 1H), 1.47 (dd, J = 6.5, 4.4 Hz, 9H). | m/z 532.2 [M + H]⁺. |
| 259 | 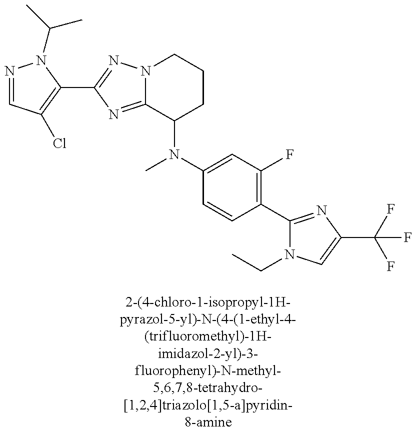 [SMILES: CCn1cc(C(F)(F)F)nc1-c1ccc(N(C)C2CCCn3nc(-c4c(Cl)cnn4C(C)C)nc32)cc1F] | ¹H NMR (400 MHz, Chloroform-d) δ 7.51 (s, 1H), 7.42 (t, J = 8.4 Hz, 1H), 7.37 (s, 1H), 6.72 (d, J = 7.8 Hz, 1H), 6.60 (d, J = 13.6 Hz, 1H), 5.20 (d, J = 5.4 Hz, 1H), 5.13 (m, 1H), 4.41 (d, J = 12.5 Hz, 1H), 4.28 (t, J = 11.0 Hz, 1H), 3.92 (q, J = 7.2 Hz, 2H), 2.81 (s, 3H), 2.38 (d, J = 11.7 Hz, 2H), 2.26-2.07 (m, 2H), 1.44 (d, J = 6.6 Hz, 3H), 1.39 (dd, J = 15.6, 6.9 Hz, 6H). | m/z: 551.2 [M + H]⁺ |
| 260 | 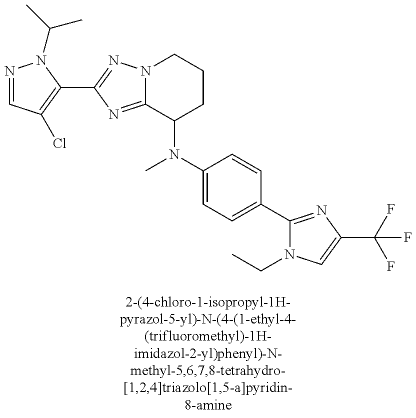 [SMILES: CCn1cc(C(F)(F)F)nc1-c1ccc(N(C)C2CCCn3nc(-c4c(Cl)cnn4C(C)C)nc32)cc1] | ¹H NMR (400 MHz, Chloroform-d) δ 7.51 (s, 1H), 7.47 (d, J = 8.7 Hz, 2H), 7.32 (s, 1H), 6.92 (d, J = 8.8 Hz, 2H), 5.25 (dd, J = 9.4, 4.9 Hz, 1H), 5.13 (m, 1H), 4.40 (d, J = 13.1 Hz, 1H), 4.26 (t, J = 9.8 Hz, 1H), 4.05 (q, J = 7.3 Hz, 2H), 2.82 (s, 3H), 2.36 (d, J = 11.0 Hz, 2H), 2.26-2.08 (m, 2H), 1.45-1.38 (m, 9H). | m/z: 533.2 [M + H]⁺ |

### Example 261. 2-(4-chloro-1-isopropyl-1H-pyrazol-5-yl)-4-(4-(1-ethyl-4-(trifluoromethyl)-1H-imidazol-2-yl)phenoxy)-4,5,6,7-tetrahydropyrazolo[1,5-α]pyridine

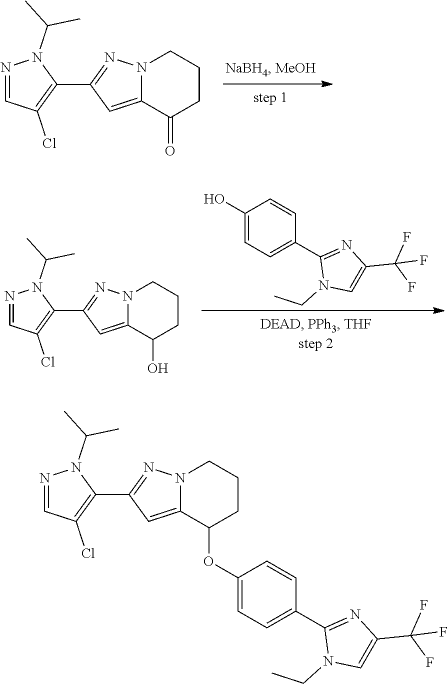

### Step 1. 2-(4-chloro-1-isopropyl-1H-pyrazol-5-yl)-4,5,6,7-tetrahydropyrazolo[1,5-α]pyridin-4-ol

To a solution of 2-(4-chloro-1-isopropyl-1H-pyrazol-5-yl)-6,7-dihydropyrazolo[1,5-α]pyridin-4(5H)-one (150 mg, 0.54 mmol) in MeOH (2 mL) was added NaBH4 (32.8 mg, 1.08 mmol) at 0° C. The resulting mixture was stirred at room temperature for 30 min then quenched with aqueous HCl (1 M, 0.1 mL). The reaction mixture was directly purified on a Biotage Isolera One (C₁₈ column, eluting with 10% to 90% MeCN/H₂O) to afford 2-(4-chloro-1-isopropyl-1H-pyrazol-5-yl)-4,5,6,7-tetrahydropyrazolo[1,5-α]pyridin-4-ol (121 mg). MS (ESI) m/z 281.2 [M+H]⁺.

### Step 2. 2-(4-chloro-1-isopropyl-1H-pyrazol-5-yl)-4-(4-(1-ethyl-4-(trifluoromethyl)-1H-imidazol-2-yl)phenoxy)-4,5,6,7-tetrahydropyrazolo[1,5-α]pyridine

To a solution of 2-(4-chloro-1-isopropyl-1H-pyrazol-5-yl)-4,5,6,7-tetrahydropyrazolo[1,5-α]pyridin-4-ol (120 mg, 0.43 mmol) in dry THF (5 mL) was added dropwise DEAD (113.2 mg, 0.65 mmol) at room temperature. After addition, 4-(1-ethyl-4-(trifluoromethyl)-1H-imidazol-2-yl)phenol (131.2 mg, 0.52 mmol) and PPh₃ (170.3 mg, 0.65 mmol) were added. The resulting mixture was stirred at room temperature for 2 h. The solvent was removed and the crude was purified on a Biotage Isolera One (C₁₈ column, eluting with 10% to 90% MeCN/H₂O) to afford Example 261 2-(4-chloro-1-isopropyl-1H-pyrazol-5-yl)-4-(4-(1-ethyl-4-(trifluoromethyl)-1H-imidazol-2-yl)phenoxy)-4,5,6,7-tetrahydropyrazolo[1,5-α]pyridine (28 mg).

Table 20. The compounds listed in Table 20 were synthesized according to Example 261 using the appropriate commercially available reagents and/or intermediates described above. Enantiomers, when generated, were separated by chiral HPLC and absolute stereochemistries were arbitrarily assigned.

TABLE 20

| Example | | | |
|---|---|---|---|
| number | Structure | ¹H NMR (δ ppm) | MS (ESI) |
| 261 | 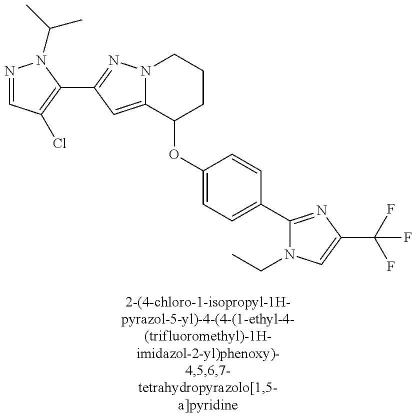 [SMILES: CCn1cc(C(F)(F)F)nc1-c1ccc(OC2CCCn3nc(-c4c(Cl)cnn4C(C)C)cc32)cc1] | ¹H NMR (400 MHz, Chloroform-d) δ 7.55 (d, J = 8.4 Hz, 2H), 7.48 (s, 1H), 7.35 (s, 1H), 7.10 (d, J = 8.4 Hz, 2H), 6.64 (s, 1H), 5.61 (t, J = 4.1 Hz, 1H), 5.23-5.13 (m, 1H), 4.42-4.32 (m, 1H), 4.23- 4.16 (m, 1H), 4.06 (q, J = 7.3 Hz, 2H), 2.53-2.06 (m, 4H), 1.51-1.40 (m, 9H). | m/z: 519.2 [M + H]⁺ |
| 262 | 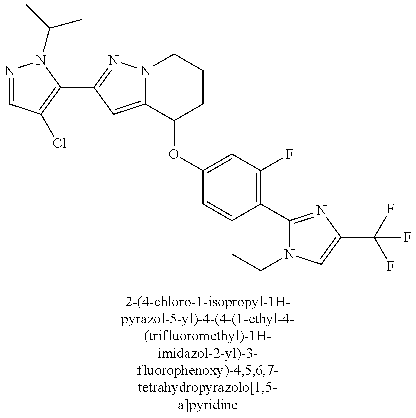 [SMILES: CCn1cc(C(F)(F)F)nc1-c1ccc(OC2CCCn3nc(-c4c(Cl)cnn4C(C)C)cc32)cc1F] | ¹H NMR (400 MHz, Chloroform-d) δ 7.57-7.47 (m, 2H), 7.40 (s, 1H), 6.93 (dd, J = 8.6, 2.3 Hz, 1H), 6.82 (dd, J = 11.6, 2.3 Hz, 1H), 6.66 (s, 1H), 5.60 (t, J = 4.5 Hz, 1H), 5.23-5.14 (m, 1H), 4.43-4.35 (m, 1H), 4.25-4.15 (m, 1H), 3.92 (q, J = 7.3 Hz, 2H), 2.53-2.41 (m, 1H), 2.36- 2.27 (m, 1H), 2.24-2.10 (m, 2H), 1.46 (dd, J = 15.3, 6.6 Hz, 6H), 1.40 (t, J = 7.3 Hz, 3H). | m/z: 537.2 [M + H]⁺ |
| 263 | 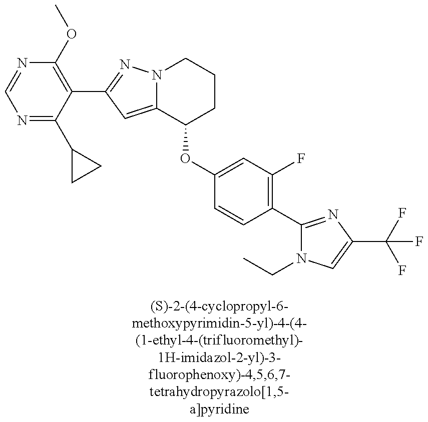 [SMILES: CCn1cc(C(F)(F)F)nc1-c1ccc(O[C@H]2CCCn3nc(-c4c(OC)ncnc4C4CC4)cc32)cc1F] | ¹H NMR (400 MHz, Chloroform-d) δ 8.55 (s, 1H), 7.52 (t, J = 8.5 Hz, 1H), 7.39 (s, 1H), 6.93 (dd, J = 8.6, 2.3 Hz, 1H), 6.82 (dd, J = 11.6, 2.3 Hz, 1H), 6.39 (s, 1H), 5.59 (t, J = 4.7 Hz, 1H), 4.39 (dt, J = 12.6, 5.3 Hz, 1H), 4.21 (m, 1H), 3.92 (d, J = 4.4 Hz, 5H), 2.51-2.40 (m, 1H), 2.25 (m, 3H), 2.15-2.08 (m, 1H), 1.39 (t, J = 7.3 Hz, 3H), 1.19-1.14 (m, 2H), 0.96-0.88 (m, 2H). | m/z: 543.2 [M + H]⁺ |
| 264 | 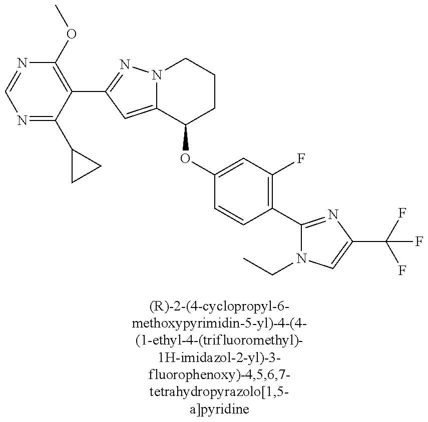 [SMILES: CCn1cc(C(F)(F)F)nc1-c1ccc(O[C@@H]2CCCn3nc(-c4c(OC)ncnc4C4CC4)cc32)cc1F] | ¹H NMR (400 MHz, Chloroform-d) δ 8.55 (s, 1H), 7.52 (t, J = 8.5 Hz, 1H), 7.39 (s, 1H), 6.93 (dd, J = 8.6, 2.3 Hz, 1H), 6.82 (dd, J = 11.6, 2.3 Hz, 1H), 6.39 (s, 1H), 5.59 (t, J = 4.7 Hz, 1H), 4.39 (dt, J = 12.6, 5.3 Hz, 1H), 4.21 (m, 1H), 3.92 (d, J = 4.4 Hz, 5H), 2.51-2.40 (m, 1H), 2.25 (m, 3H), 2.15-2.08 (m, 1H), 1.39 (t, J = 7.3 Hz, 3H), 1.19-1.14 (m, 2H), 0.96-0.88 (m, 2H). | m/z: 543.2 [M + H]⁺ |

### Example 265. 2-(4-chloro-1-isopropyl-1H-pyrazol-5-yl)-4-(4-(1-ethyl-4-(trifluoromethyl)-1H-imidazol-2-yl)benzyl)-6,7-dihydro-4H-pyrazolo[5,1-c][1,4]oxazine

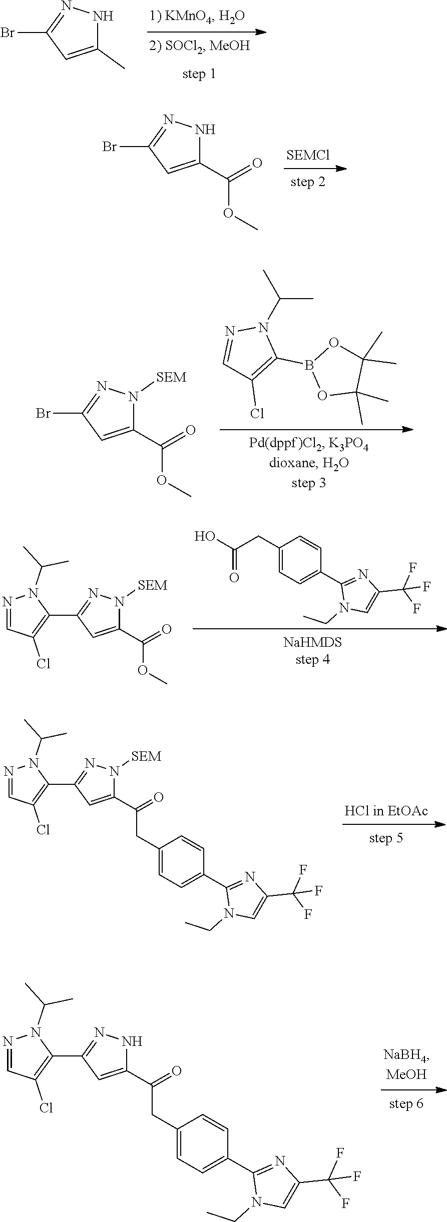

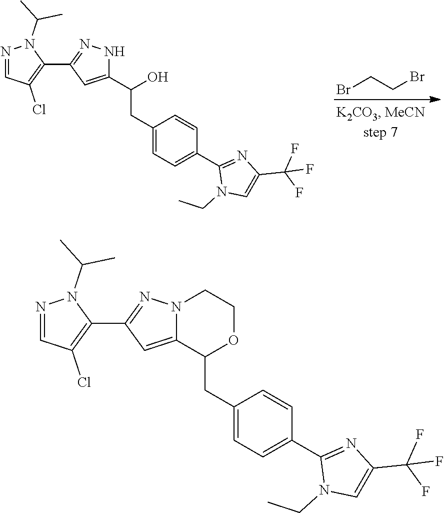

### Step 1. methyl 3-bromo-1H-pyrazole-5-carboxylate

methyl 3-bromo-1H-pyrazole-5-carboxylate was prepared following step 1 of Example 237. MS (ESI) m/z 205.0 [M+H]⁺.

### Step 2. methyl 3-bromo-14(2-(trimethylsilyl)ethoxy)methyl)-1H-pyrazole-5-carboxylate

To a solution of methyl 3-bromo-1H-pyrazole-5-carboxylate (1.47 g, 7.21 mmol) in THF (10 mL) was added NaH (432 mg, 10.81 mmol 60% in mineral oil) at 0° C. The mixture was stirred at room temperature for 30 min before 2-(Trimethylsilyl)ethoxymethyl chloride (1.80 g, 10.81 mmol) was added. The resulting mixture was stirred at room temperature for 2 h. The solvent was removed in vacuo and the crude residue was purified by silica gel chromatography (eluting with 1/5 EtOAc/PE) to afford methyl 3-bromo-1-((2-(trimethylsilyl)ethoxy)methyl)-1H-pyrazole-5-carboxylate (1.5 g). ¹H NMR (400 MHz, Chloroform-d) δ 6.90 (s, 1H), 5.58 (s, 2H), 3.95 (s, 3H), 3.69-3.57 (m, 2H), 0.99-0.83 (m, 2H), 0.00 (s, 9H). MS (ESI) m/z 335.0, 337.0 [M+H]⁺.

### Step 3. methyl 4′-chloro-2′-isopropyl-1-((2-(trimethylsilyl)ethoxy)methyl)-1H,2′H-[3,3′-bipyrazole]-5-carboxylate

methyl 4′-chloro-2′-isopropyl-1-((2-(trimethylsilyl)ethoxy)methyl)-1H,2′H-[3,3′-bipyrazole]-5-carboxylate was synthesized from methyl 3-bromo-1-((2-(trimethylsilyl)ethoxy)methyl)-1H-pyrazole-5-carboxylate and 4-chloro-1-isopropyl-5-(4,4,5,5-tetramethyl-1,3,2-dioxaborolan-2-yl)-1H-pyrazole following step 3 of Example 1. MS (ESI) m/z 399.2 [M+H]⁺.

### Step 4. 1-(4′-chloro-2′-isopropyl-1-((2-(trimethylsilyl)ethoxy)methyl)-1H,2′H-[3,3′-bipyrazol]-5-yl)-2-(4-(1-ethyl-4-(trifluoromethyl)-1H-imidazol-2-yl)phenyl)ethan-1-one

To a solution of methyl 4′-chloro-2′-isopropyl-14(2-(trimethylsilyl)ethoxy)methyl)-1H,2′H-[3,3′-bipyrazole]-5-carboxylate (285 mg, 0.72 mmol) and 2-(4-(1-ethyl-4-(trifluoromethyl)-1H-imidazol-2-yl)phenyl) acetic acid (235 mg, 0.79 mmol) in dry DMF (7 mL) was added NaHMDS (1 M in THF, 2.88 mL, 2.88 mmol) at −10° C. under an atmosphere of N₂. The resulting mixture was stirred at −10° C. for 3 h then quenched with saturated aqueous NH₄Cl (20 mL). The mixture was extracted with EtOAc (30 mL×3). The combined organic phase were washed with water and brine, dried over anhydrous Na₂SO₄, concentrated and purified by silica gel chromatography (eluting with 1/5 EtOAc/PE) to afford 1-(4′-chloro-2′-isopropyl-1-((2-(trimethylsilyl)ethoxy)methyl)-1H,2′H-[3,3′-bipyrazol]-5-yl)-2-(4-(1-ethyl-4-(trifluoromethyl)-1H-imidazol-2-yl)phenyl)ethan-1-one (200 mg). MS (ESI) m/z 621.2 [M+H]⁺.

### Step 5. 1-(4′-chloro-2′-isopropyl-1H,2′H-[3,3′-bipyrazol]-5-yl)-2-(4-(1-ethyl-4-(trifluoromethyl)-1H-imidazol-2-yl) phenyl)ethan-1-one

1-(4′-chloro-2′-isopropyl-1H,2′H-[3,3′-bipyrazol]-5-yl)-2-(4-(1-ethyl-4-(trifluoromethyl)-1H-imidazol-2-yl)phenyl)ethan-1-one was synthesized from 1-(4′-chloro-2′-isopropyl-1-((2-(trimethylsilyl)ethoxy)methyl)-1H,2′H-[3,3′-bipyrazol]-5-yl)-2-(4-(1-ethyl-4-(trifluoromethyl)-1H-imidazol-2-yl)phenyl)ethan-1-one according to step 4 of Example 149. MS (ESI) m/z 491.1 [M+H]⁺.

### Step 6. 1-(4′-chloro-2′-isopropyl-1H,2′H-[3,3′-bipyrazol]-5-yl)-2-(4-(1-ethyl-4-(trifluoromethyl)-1H-imidazol-2-yl) phenyl)ethan-1-ol

1-(4′-chloro-2′-isopropyl-1H,2′H-[3,3′-bipyrazol]-5-yl)-2-(4-(1-ethyl-4-(trifluoromethyl)-1H-imidazol-2-yl)phenyl)ethan-1-ol was synthesized from 1-(4′-chloro-2′-isopropyl-1H,2′H-[3,3′-bipyrazol]-5-yl)-2-(4-(1-ethyl-4-(trifluoromethyl)-1H-imidazol-2-yl)phenyl)ethan-1-one according to step 1 of Example 261. MS (ESI) m/z 493.2 [M+H]⁺.

### Step 7. 2-(4-chloro-1-isopropyl-1H-pyrazol-5-yl)-4-(4-(1-ethyl-4-(trifluoromethyl)-1H-imidazol-2-yl) benzyl)-6,7-dihydro-4H-pyrazolo[5,1-c][1,4]oxazine

A mixture of 1-(4′-chloro-2′-isopropyl-1H,2′H-[3,3′-bipyrazol]-5-yl)-2-(4-(1-ethyl-4-(trifluoromethyl)-1H-imidazol-2-yl)phenyl)ethan-1-ol (135 mg, 0.27 mmol), 1,2-dibromoethane (77.32 mg, 0.41 mmol) and K₂CO₃ (138 mg, 1.08 mmol) in DMF (5 mL) was heated at 100° C. in a sealed tube overnight. The reaction mixture was diluted with EtOAc (50 mL), washed by brine, dried over anhydrous Na₂SO₄, concentrated and purified by prep-TLC (eluting with 1/2 EtOAc/PE) and prep-HPLC (condition 2) to afford Example 265 2-(4-chloro-1-isopropyl-1H-pyrazol-5-yl)-4-(4-(1-ethyl-4-(trifluoromethyl)-1H-imidazol-2-yl)benzyl)-6,7-dihydro-4H-pyrazolo[5,1-c][1,4]oxazine (18.9 mg). ¹H NMR (400 MHz, Methanol-d₄) δ 7.76 (s, 1H), 7.52 (s, 5H), 6.44 (s, 1H), 5.17 (dd, J=7.9, 4.6 Hz, 1H), 5.11-5.04 (m, 1H), 4.34 (dt, J=11.9, 3.3 Hz, 1H), 4.23-4.16 (m, 2H), 4.14-3.97 (m, 3H), 3.37 (dd, J=14.3, 4.6 Hz, 1H), 3.23 (dd, J=14.3, 7.8 Hz, 1H), 1.43-1.34 (m, 9H). MS (ESI) m/z 519.2 [M+H]⁺.

### Example 266. 5-(4-((4-(1-ethyl-4-(trifluoromethyl)-1H-imidazol-2-yl)-3-fluorophenyl)amino)-4,5,6,7-tetrahydropyrazolo[1,5-α]pyridin-2-yl)-1-isopropyl-1H-pyrazole-4-carbonitrile

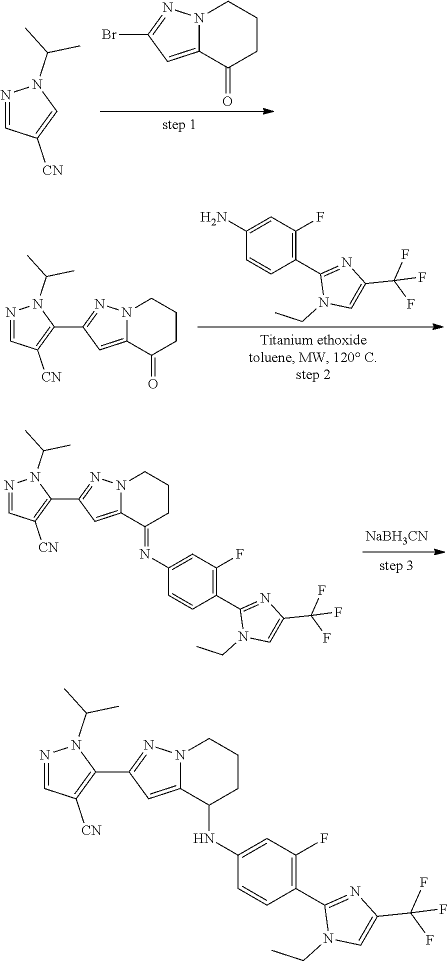

### Step 1. 1-isopropyl-5-(4-oxo-4,5,6,7-tetrahydropyrazolo[1,5-α]pyridin-2-yl)-1H-pyrazole-4-carbonitrile

A mixture of 1-isopropyl-1H-pyrazole-4-carbonitrile (135 mg, 1 mmol), 2-bromo-6,7-dihydropyrazolo[1,5-α]pyridin-4(5H)-one (214 mg, 1.5 mmol), Pd(OAc)₂ (23 mg, 0.1 mmol), catacxium A (72 mg, 0.2 mmol) and KOAc (490 mg, 5 mmol) in 2-methyl-2-butanol (8 mL) was heated at 120° C. overnight in a sealed tube. After cooling to ambient temperature, the mixture was filtered through celite and the filtrate concentrated under vacuum. The residue was purified by silica gel chromatography (eluting with 1/5 EtOAc/PE) to afford 1-isopropyl-5-(4-oxo-4,5,6,7-tetrahydropyrazolo[1,5-α]pyridin-2-yl)-1H-pyrazole-4-carbonitrile (160 mg). MS (ESI) m/z 270.1 [M+H]⁺.

### Step 2 and step 3. 5-(4-((4-(1-ethyl-4-(trifluoromethyl)-1H-imidazol-2-yl)-3-fluorophenyl)amino)-4,5,6,7-tetrahydropyrazolo[1,5-α]pyridin-2-yl)-1-isopropyl-1H-pyrazole-4-carbonitrile

Example 266 5-(4-((4-(1-ethyl-4-(trifluoromethyl)-1H-imidazol-2-yl)-3-fluorophenyl)amino)-4,5,6,7-tetrahydro-pyrazolo[1,5-α]pyridin-2-yl)-1-isopropyl-1H-pyrazole-4-carbonitrile was prepared from 1-isopropyl-5-(4-oxo-4,5,6,7-tetrahydropyrazolo[1,5-α]pyridin-2-yl)-1H-pyrazole-4-carbonitrile and 4-(1-ethyl-4-(trifluoromethyl)-1H-imidazol-2-yl)-3-fluoroaniline (intermediate BB-15) following step 6 and 7 of Example 237. ¹H NMR (400 MHz, Chloroform-d) δ 7.69 (s, 1H), 7.26-7.20 (m, 2H), 6.65 (d, J=0.7 Hz, 1H), 6.42 (dd, J=8.5, 2.3 Hz, 1H), 6.33 (dd, J=12.5, 2.3 Hz, 1H), 5.27 (m, 1H), 4.70 (d, J=6.5 Hz, 1H), 4.37 (d, J=8.2 Hz, 1H), 4.14 (m, 2H), 3.82 (q, J=7.3 Hz, 2H), 2.29-2.12 (m, 2H), 1.83-1.69 (m, 2H), 1.39 (dd, J=6.6, 1.9 Hz, 6H), 1.28 (t, J=7.3 Hz, 3H). MS (ESI) m/z 527.2 [M+H]⁺.

### Example 267. 2-(4-chloro-1-isopropyl-1H-pyrazol-5-yl)-4-(4-(1-ethyl-4-(trifluoromethyl)-1H-imidazol-2-yl)-3-fluorobenzyl)-4,5,6,7-tetrahydropyrazolo[1,5-α]pyrimidin-6-ol

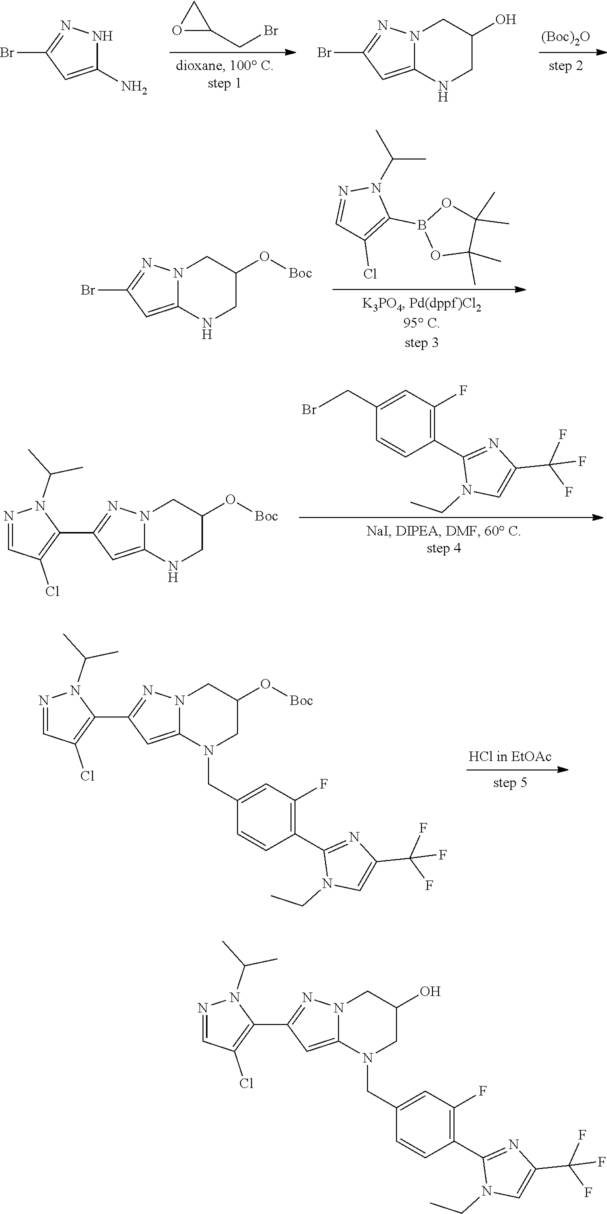

### Step 1. 2-bromo-4,5,6,7-tetrahydropyrazolo[1,5-α]pyrimidin-6-ol

To a solution of 3-bromo-1H-pyrazol-5-amine (8.0 g, 49.38 mmol) in dioxane (45 mL) was added 2-(bromomethyl) oxirane (10.0 g, 74.07 mmol) at room temperature. The resulting mixture was heated at 100° C. for 16 h. The mixture was concentrated and purified by silica gel chromatography (eluting with 1/1 EtOAc/PE) to afford 2-bromo-4,5,6,7-tetrahydropyrazolo[1,5-α]pyrimidin-6-ol (2.3 g). MS (ESI) m/z 217.9 [M+H]⁺.

### Step 2. 2-bromo-4,5,6,7-tetrahydropyrazolo[1,5-α]pyrimidin-6-yl tert-butyl carbonate

To a solution of 2-bromo-4,5,6,7-tetrahydropyrazolo[1,5-α]pyrimidin-6-ol (1.7 g, 7.83 mmol) in DCM (45 mL) was added di-tert-butyl dicarbonate (2.05 g, 9.40 mmol), TEA (2.4 g, 23.5 mmol) and DMAP (191 mg, 1.56 mmol) at room temperature. The resulting mixture was stirred at room temperature for 1 h. The solution was concentrated and purified by silica gel chromatography (eluting with 1/3 EtOAc/PE) to afford 2-bromo-4,5,6,7-tetrahydropyrazolo[1,5-α]pyrimidin-6-yl tert-butyl carbonate (2.2 g). MS (ESI) m/z 318.1 [M+H]⁺.

### Step 3. tert-butyl (2-(4-chloro-1-isopropyl-1H-pyrazol-5-yl)-4,5,6,7-tetrahydropyrazolo[1,5-α]pyrimidin-6-yl) carbonate

To a solution of 2-bromo-4,5,6,7-tetrahydropyrazolo[1,5-α]pyrimidin-6-yl tert-butyl carbonate (1.0 g, 3.15 mmol) in THF (5 mL) was added 4-chloro-1-isopropyl-5-(4,4,5,5-tetramethyl-1,3,2-dioxaborolan-2-yl)-1H-pyrazole (2.6 g, 9.46 mmol), Pd(dppf)Cl₂ (231 mg, 0.31 mmol) and K₃PO₄ (1.3 g, 6.31 mmol) at room temperature. After bubbling with N₂, the resulting mixture was heated at 95° C. for 1 h in microwave reactor. The mixture was filtered through celite, concentrated and purified by silica gel chromatography (eluting with 1/1 EtOAc/PE) to afford tert-butyl (2-(4-chloro-1-isopropyl-1H-pyrazol-5-yl)-4,5,6,7-tetrahydropyrazolo[1,5-α]pyrimidin-6-yl) carbonate (690 mg). ¹H NMR (400 MHz, DMSO-d₆) δ 7.58 (s, 1H), 6.41 (s, 1H), 5.66 (s, 1H), 5.22 (m, 1H), 5.14 (s, 1H), 4.36 (dd, J=13.8, 3.4 Hz, 1H), 4.15 (d, J=13.8 Hz, 1H), 1.42 (s, 9H), 1.35 (dd, J =10.1, 6.6 Hz, 6H). MS (ESI) m/z 382.2 [M+H]⁺.

### Step 4. tert-butyl (2-(4-chloro-1-isopropyl-1H-pyrazol-5-yl)-4-(4-(1-ethyl-4-(trifluoromethyl)-1H-imidazol-2-yl)-3-fluorobenzyl)-4,5,6,7-tetrahydropyrazolo[1,5-α]pyrimidin-6-yl) carbonate

To a solution of tert-butyl (2-(4-chloro-1-isopropyl-1H-pyrazol-5-yl)-4,5,6,7-tetrahydropyrazolo[1,5-α]pyrimidin-6-yl) carbonate (690 mg, 1.81 mmol) in DMF (10 mL) was added 2-(4-(bromomethyl)-2-fluorophenyl)-1-ethyl-4-(trifluoromethyl)-1H-imidazole (1.3 g, 3.62 mmol), NaI (543 mg, 3.62 mmol) and DIPEA (467 mg, 3.62 mmol) at room temperature. The resulting mixture was heated at 60° C. for 16 h. The mixture was diluted with water (50 mL) and extracted with EtOAc (50 mL×3). The combined organic phase were washed with water and brine, dried over anhydrous Na₂SO₄, concentrated and purified by silica gel chromatography (eluting with 1/1 EtOAc/PE) to afford tert-butyl(2-(4-chloro-1-isopropyl-1H-pyrazol-5-yl)-4-(4-(1-ethyl-4-(trifluoromethyl)-1H-imidazol-2-yl)-3-fluorobenzyl)-4,5,6,7-tetrahydropyrazolo[1,5-α]pyrimidin-6-yl) carbonate (770 mg). MS (ESI) m/z 652.2 [M+H]⁺

### Step 5. 2-(4-chloro-1-isopropyl-1H-pyrazol-5-yl)-4-(4-(1-ethyl-4-(trifluoromethyl)-1H-imidazol-2-yl)-3-fluorobenzyl)-4,5,6,7-tetrahydropyrazolo[1,5-α]pyrimidin-6-ol

To a solution of tert-butyl (2-(4-chloro-1-isopropyl-1H-pyrazol-5-yl)-4-(4-(1-ethyl-4-(trifluoromethyl)-1H-imidazol-2-yl)-3-fluorobenzyl)-4,5,6,7-tetrahydropyrazolo[1,5-α]pyrimidin-6-yl) carbonate (60 mg, 0.092 mmol) was added HCl EtOAc solution (4 M, 5mL) at room temperature. The resulting mixture was stirred at room temperature for 1 h. The solution was neutralized with saturated aqueous Na₂CO₃ and extracted with EtOAc (50 mL×3). The combined organic phases were washed with water and brine, dried over anhydrous Na₂SO₄, concentrated and purified by silica gel chromatography (eluting with 1/1 EtOAc/PE) to afford Example 267 2-(4-chloro-1-isopropyl-1H-pyrazol-5-yl)-4-(4-(1-ethyl-4-(trifluoromethyl)-1H-imidazol-2-yl)-3-fluorobenzyl)-4,5,6,7-tetrahydropyrazolo[1,5-α]pyrimidin-6-ol] (18.7 mg). ¹H NMR (400 MHz, Methanol-d₄) δ 7.88-7.85 (m, 1H), 7.56-7.50 (m, 2H), 7.44 (d, J=9.3 Hz, 2H), 5.74 (s, 1H), 5.06 (m, 1H), 4.59 (s, 2H), 4.46 (dt, J=6.1, 3.1 Hz, 1H), 4.32 (dd, J=12.8, 3.6 Hz, 1H), 4.13 (dd, J=12.8, 2.4 Hz, 1H), 3.98 (q, J=7.3 Hz, 2H), 3.48 (dd, J=11.9, 2.0 Hz, 1H), 3.30 (dd, J=4.8, 1.5 Hz, 1H), 1.42 (dd, J=6.6, 2.5 Hz, 6H), 1.36 (t, J=7.3 Hz, 3H). MS (ESI) m/z 552.2 [M+H]⁺.

### Example 268. 3-chloro-4-(4-(1-ethyl-4-(trifluoromethyl)-1H-imidazol-2-yl)benzyl)-2-(1-isopropyl-1H-pyrazol-5-yl)-6,7-dihydropyrazolo[1,5-α]pyrimidin-5(4H)-one

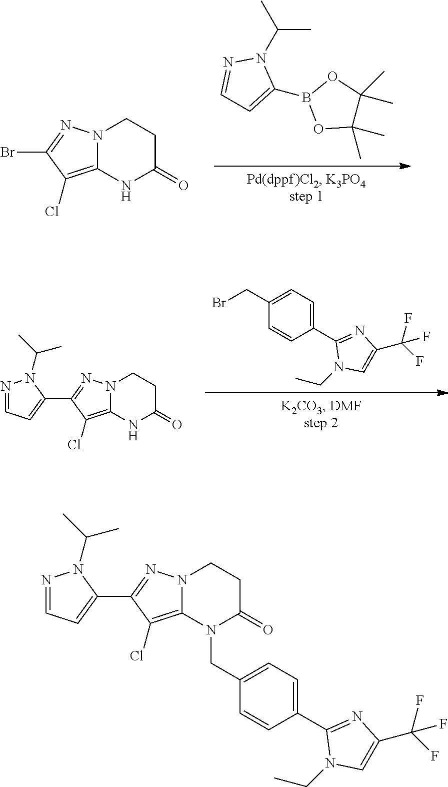

Example 268 3-chloro-4-(4-(1-ethyl-4-(trifluoromethyl)-1H-imidazol-2-yl)benzyl)-2-(1-isopropyl-1H-pyrazol-5-yl)-6,7-dihydropyrazolo[1,5-α]pyrimidin-5(4H)-one was prepared from 2-bromo-3-chloro-6,7-dihydropyrazolo[1,5-α]pyrimidin-5(4H)-one (prepared in Example 147) following Example 77. ¹H NMR (400 MHz, Chloroform-d) δ 7.57 (d, J=1.7 Hz, 1H), 7.54 (d, J=8.2 Hz, 2H), 7.40 (d, J=8.2 Hz, 2H), 7.35 (s, 1H), 6.54 (d, J=1.8 Hz, 1H), 5.41 (s, 2H), 4.93 (m, 1H), 4.39 (t, J=6.8 Hz, 2H), 4.05 (q, J=7.3 Hz, 2H), 3.06 (t, J=6.8 Hz, 2H), 1.47 (d, J=6.6 Hz, 6H), 1.43 (t, J=7.3 Hz, 3H). MS (ESI) m/z 532.2 [M+H]⁺.

### Example 269. 3-chloro-N-(4-(1-ethyl-4-(trifluoromethyl)-1H-imidazol-2-yl)-3-fluorophenyl)-2-(1-isopropyl-1H-pyrazol-5-yl)-4,5,6,7-tetrahydropyrazolo[1,5-α]pyridin-4-amine

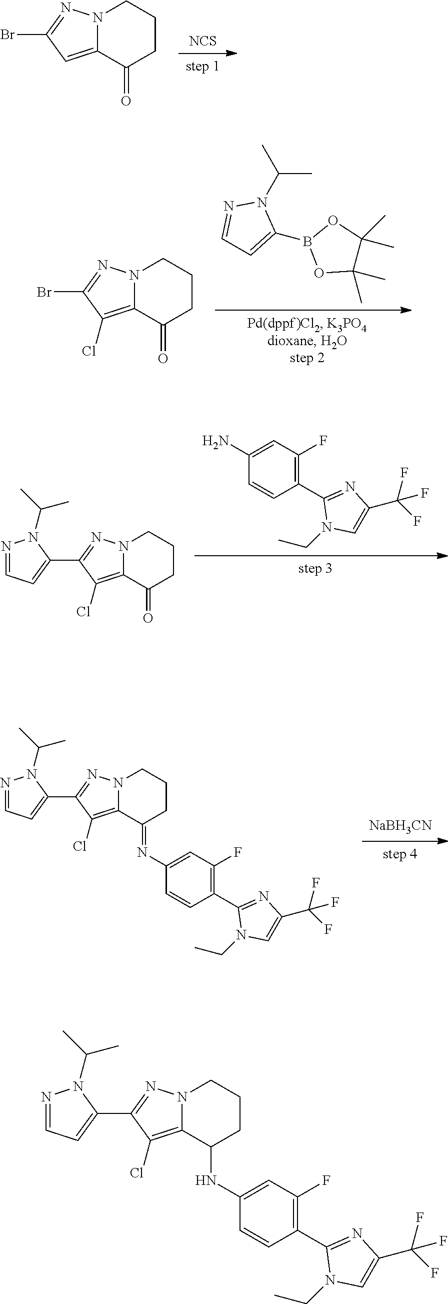

### Step 1. 2-bromo-3-chloro-6,7-dihydropyrazolo[1,5-α]pyridin-4(5H)-one

2-bromo-3-chloro-6,7-dihydropyrazolo[1,5-α]pyridin-4(5H)-one was prepared from 2-bromo-6,7-dihydropyrazolo-[1,5-α]pyridin-4(5H)-one according to step 1 of Example 147. MS (ESI) m/z 248.9, 250.9 [M+H]⁺.

### Step 2. 3-chloro-2-(1-isopropyl-1H-pyrazol-5-yl)-6,7-dihydropyrazolo[1,5-α]pyridin-4(5H)-one

A mixture of 2-bromo-3-chloro-6,7-dihydropyrazolo[1,5-α]pyridin-4(5H)-one (400 mg, 1.61 mmol), 1-isopropyl-5-(4,4, 5,5-tetramethyl-1,3 ,2-dioxaborolan-2-yl)-1H-pyrazole (492 mg, 1.94 mmol), K₃PO₄ (684 mg, 3.2 mmol) and Pd(dppf)Cl₂ in dioxane (10 mL) and H₂O (1 mL) was heated at 100° C. for 3 h under an atmosphere of N₂. After cooling to room temperature, the mixture was filtered through celite, concentrated and purified by column chromatography on silica gel (eluting with 1/4 EtOAc/PE) to afford 3-chloro-2-(1-isopropyl-1H-pyrazol-5-yl)-6,7-dihydropyrazolo[1,5-α]pyridin-4(5H)-one (498 mg). MS (ESI) m/z 248.9, 250.9 [M+H]⁺.

### Step 3 and step 4. 3-chloro-N-(4-(1-ethyl-4-(trifluoromethyl)-1H-imidazol-2-yl)-3-fluorophenyl)-2-(1-isopropyl-1H-pyrazol-5-yl)-4,5,6,7-tetrahydropyrazolo[1,5-α]pyridin-4-amine

Example 269 3-chloro-N-(4-(1-ethyl-4-(trifluoromethyl)-1H-imidazol-2-yl)-3-fluorophenyl)-2-(1-isopropyl-1H-pyrazol-5-yl)-4,5,6,7-tetrahydropyrazolo[1,5-α]pyridin-4-amine was prepared according to step 6 and step 7 of Example 237. 41 NMR (400 MHz, Chloroform-d) δ 7.60 (d, J=1.9 Hz, 1H), 7.40-7.35 (m, 2H), 6.60 (d, J=1.9 Hz, 1H), 6.55 (dd, J=8.4, 2.3 Hz, 1H), 6.45 (dd, J=12.4, 2.3 Hz, 1H), 5.08 (m, 1H), 4.85 (t, J=4.1 Hz, 1H), 4.34 (dt, J=12.9, 4.6 Hz, 1H), 4.09 (ddd, J=13.0, 10.3, 4.8 Hz, 1H), 3.93 (q, J=7.3 Hz, 2H), 2.27-2.19 (m, 2H), 2.05-1.98 (m, 2H), 1.50 (dd, J=6.6, 3.1 Hz, 6H), 1.40 (t, J=7.3 Hz, 3H). MS (ESI) m/z 536.2 [M+H]⁺.

### Example 270. 2-(4-chloro-1-isopropyl-1H-pyrazol-5-yl)-4-((4-(1-ethyl-4-(trifluoromethyl)-1H-imidazol-2-yl)benzyl)oxy)-4,5,6,7-tetrahydropyrazolo[1,5-α]pyridine

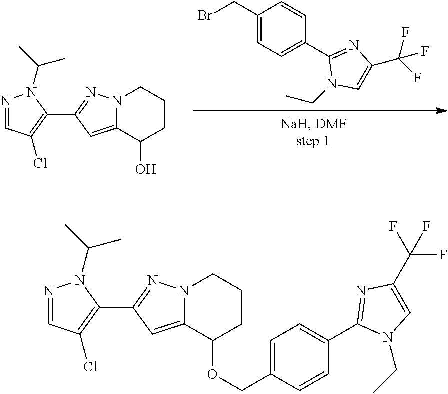

### Step 1. 2-(4-chloro-1-isopropyl-1H-pyrazol-5-yl)-4-((4-(1-ethyl-4-(trifluoromethyl)-1H-imidazol-2-yl)benzyl)oxy)-4,5,6,7-tetrahydropyrazolo[1,5-α]pyridine

To a solution of 2-(4-chloro-1-isopropyl-1H-pyrazol-5-yl)-4,5,6,7-tetrahydropyrazolo[1,5-α]pyridin-4-ol (100 mg, 0.36 mmol) in dry DMF (3 mL) was added NaH (60% in mineral oil, 17 mg, 0.43 mmol) at 0° C. The mixture was stirred at 0° C. for 30 min then 2-(4-(bromomethyl)phenyl)-1-ethyl-4-(trifluoromethyl)-1H-imidazole (178 mg, 0.54 mmol) was added. The resulting mixture was allowed to warm to room temperature and stirred for overnight. The mixture was poured into water (20 mL) and extracted with EtOAc (200 mL×3). The organic layers were combined, dried over anhydrous Na₂SO₄ and concentrated under vacuum. The residue was purified by prep-TLC (eluting with 1/2 EtOAc/PE) to afford Example 270 2-(4-chloro-1-isopropyl-1H-pyrazol-5-yl)-4-((4-(1-ethyl-4-(trifluoromethyl)-1H-imidazol-2-yl)benzyl)oxy)-4,5,6,7-tetrahydropyrazolo[1,5-α]pyridine (45 mg). ¹H NMR (400 MHz, Chloroform-d) δ 7.58 (d, J=8.1 Hz, 2H), 7.52 — 7.45 (m, 3H), 7.37 (s, 1H), 6.69 (s, 1H), 5.25-5.16 (m, 1H), 4.72 (d, J=10.3 Hz, 3H), 4.33 (dd, J=11.3, 6.5 Hz, 1H), 4.06 (d, J=7.3 Hz, 3H), 2.45 (d, J=12.7 Hz, 1H), 2.21 (d, J=7.0 Hz, 1H), 2.08-1.99 (m, 2H), 1.50-1.41 (m, 9H). MS (ESI) m/z 533.2 [M+H]⁺.

### Example 271. 2-(4-chloro-1-isopropyl-1H-pyrazol-5-yl)-4-(3-chloro-4-(3-(difluoromethoxy)pyridin-2-yl)benzyl)-6,7-dihydropyrazolo[1,5-α]pyrimidin-5(4H)-one

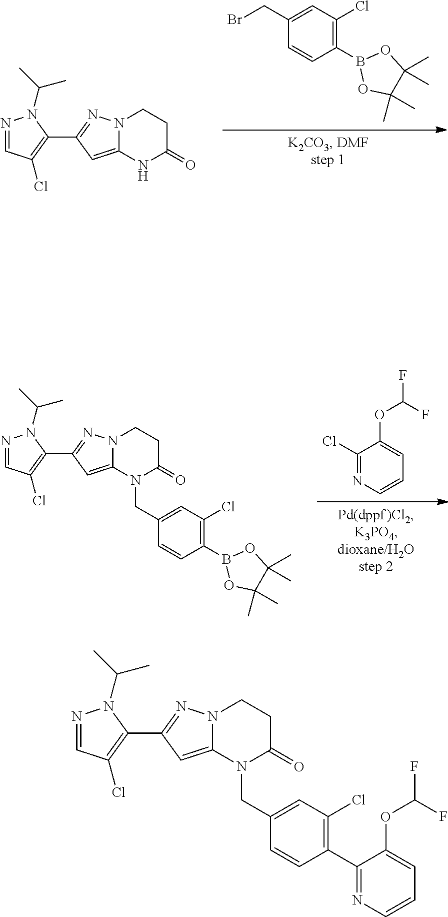

### Step 1. 2-(4-chloro-1-isopropyl-1H-pyrazol-5-yl)-4-(3-chloro-4-(4,4,5,5-tetramethyl-1,3,2-dioxaborolan-2-yl) benzyl)-6,7-dihydropyrazolo[1,5-α]pyrimidin-5(4H)-one

To a solution of 2-(4-chloro-1-isopropyl-1H-pyrazol-5-yl)-6,7-dihydropyrazolo[1,5-α]pyrimidin-5(4H)-one (2 g, 7.17 mmol) in DMF (20 mL) was added 2-(4-(bromomethyl)-2-chlorophenyl)-4,4,5,5-tetramethyl-1,3,2-dioxaborolane (2.37 g, 7.17 mmol) and K₂CO₃ (2g, 14.35 mmol). The resulting mixture was heated at 60° C. for 2 h. After cooling to ambient temperature, the mixture was poured into water (150 mL) and extracted with EtOAc (100 mL×3). The organic layers were combined, dried over anhydrous Na₂SO₄ and concentrated under vacuum. The residue was purified by silica gel chromatography (eluting with 1/2 EtOAc/PE) to afford 2-bromo-4-(4-(1-methyl-4-(trifluoromethyl)-1H-imidazol-2-yl)benzyl)-6,7-dihydropyrazolo[1,5-α]pyrimidin-5(4H)-one (2.3 g). MS (ESI) m/z 530.2 [M+H]⁺.

### Step 2. 2-(4-chloro-1-isopropyl-1H-pyrazol-5-yl)-4-(3-chloro-4-(3-(difluoromethoxy)pyridin-2-yl)benzyl)-6,7-dihydropyrazolo[1,5-α]pyrimidin-5(4H)-one

To a solution of 2-(4-chloro-1-isopropyl-1H-pyrazol-5-yl)-4-(3-chloro-4-(4,4,5,5-tetramethyl-1,3,2-dioxaborolan-2-yl)benzyl)-6,7-dihydropyrazolo[1,5-α]pyrimidin-5(4H)-one (80 mg, 0.15 mmol) in a mixture of dioxane (3 mL) and H₂O (0.3 mL) was added 2-chloro-3-(difluoromethoxy)pyridine (27.2 mg, 0.15 mmol), Pd(dppf)Cl₂ (11 mg, 0.015 mmol) and K₃PO₄ (96 mg, 0.45 mmol). The mixture was heated at 110° C. for 1 h in a microwave reactor. After cooling to ambient temperature, the mixture was filtered through celite and the filtrate concentrated under vacuum. The residue was purified by prep-TLC (eluting with 1/1 PE/EtOAc) to afford Example 271 2-(4-chloro-1-isopropyl-1H-pyrazol-5-yl)-4-(3-chloro-4-(3-(difluoromethoxy)pyridin-2-yl)benzyl)-6,7-dihydropyrazolo[1,5-α]pyrimidin-5(4H)-one (110 mg).

Table 21. The compounds listed in Table 21 were synthesized according to Example 271 using the appropriate commercially available reagents and/or intermediates described above.

TABLE 21

| Example | | | |
|---|---|---|---|
| number | Structure | ¹H NMR (δ ppm) | MS (ESI) |
| 271 | 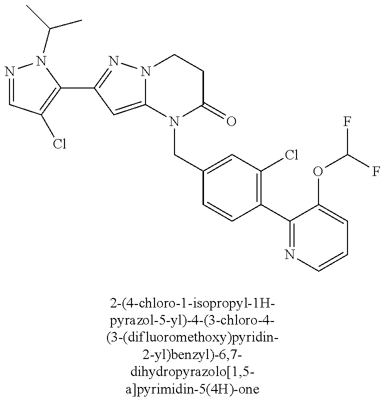 [SMILES: CC(C)n1ncc(Cl)c1-c1cc2n(n1)CCC(=O)N2Cc1ccc(-c2ncccc2OC(F)F)c(Cl)c1] | ¹H NMR (400 MHz, Chloroform-d) δ 8.53 (dd, J = 4.7, 1.1 Hz, 1H), 7.65-7.58 (m, 1H), 7.51-7.44 (m, 2H), 7.40-7.31 (m, 3H), 6.41 (t, J = 72.6 Hz, 1H), 6.17 (s, 1H), 5.16 (m, 1H), 5.01 (s, 2H), 4.43 (t, J = 7.1 Hz, 2H), 3.10 (t, J = 7.1 Hz, 2H), 1.45 (d, J = 6.6 Hz, 6H). | m/z: 547.1 [M + H]⁺ |
| 272 | 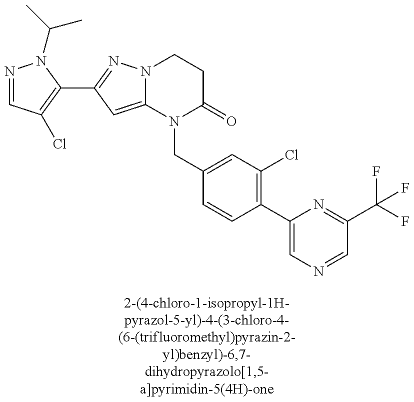 [SMILES: CC(C)n1ncc(Cl)c1-c1cc2n(n1)CCC(=O)N2Cc1ccc(-c2cncc(C(F)(F)F)n2)c(Cl)c1] | ¹H NMR (400 MHz, Chloroform-d) δ 9.20 (s, 1H), 8.93 (s, 1H), 7.67 (d, J = 8.0 Hz, 1H), 7.51 (d, J = 12.0 Hz, 2H), 7.42 (d, J = 8.0 Hz, 1H), 6.18 (s, 1H), 5.25-5.11 (m, 1H), 5.04 (s, 2H), 4.45 (t, J = 7.1 Hz, 2H), 3.12 (t, J = 7.1 Hz, 2H), 1.46 (d, J = 6.6 Hz, 6H). | m/z: 550.1 [M + H] |
| 273 | 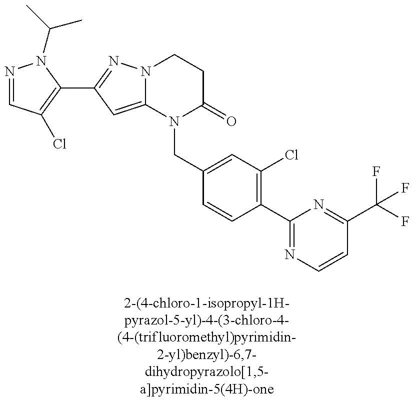 [SMILES: CC(C)n1ncc(Cl)c1-c1cc2n(n1)CCC(=O)N2Cc1ccc(-c2nccc(C(F)(F)F)n2)c(Cl)c1] | ¹H NMR (400 MHz, Chloroform-d) δ 9.11 (d, J = 5.0 Hz, 1H), 7.82 (d, J = 8.0 Hz, 1H), 7.61 (d, J = 5.0 Hz, 1H), 7.50 (d, J = 12.1 Hz, 2H), 7.38 (d, J = 7.9 Hz, 1H), 6.17 (s, 1H), 5.16 (m, 1H), 5.03 (s, 2H), 4.43 (t, J = 7.1 Hz, 2H), 3.10 (t, J = 7.1 Hz, 2H), 1.45 (d, J = 6.6 Hz, 6H). | m/z: 550.1 [M + H]⁺ |
| 274 | 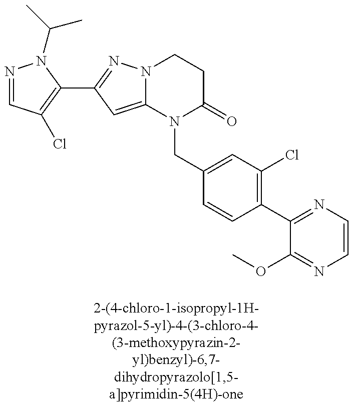 [SMILES: COc1nccnc1-c1ccc(CN2C(=O)CCn3nc(-c4c(Cl)cnn4C(C)C)cc32)cc1Cl] | ¹H NMR (400 MHz, Chloroform-d) δ 8.21 (d, J = 2.7 Hz, 1H), 8.15 (d, J = 2.7 Hz, 1H), 7.49 (s, 1H), 7.46 (d, J = 1.6 Hz, 1H), 7.42-7.32 (m, 2H), 6.18 (s, 1H), 5.17 (m, 1H), 5.01 (s, 2H), 4.44 (t, J = 7.0 Hz, 2H), 3.95 (s, 3H), 3.10 (t, J = 7.1 Hz, 2H), 1.46 (d, J = 6.6 Hz, 6H). | m/z: 512.1 [M + H]⁺ |
| 275 | 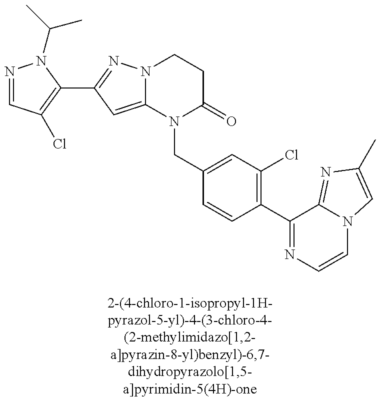 [SMILES: Cc1cn2ccnc(-c3ccc(CN4C(=O)CCn5nc(-c6c(Cl)cnn6C(C)C)cc54)cc3Cl)c2n1] | ¹H NMR (400 MHz, Chloroform-d) δ 8.21 (d, J = 4.5 Hz, 1H), 8.11 (d, J = 4.4 Hz, 1H), 7.81 (d, J = 7.8 Hz, 1H), 7.77-7.66 (m, 3H), 7.60 (d, J = 7.9 Hz, 1H), 6.42 (s, 1H), 5.36 (m, 1H), 5.20 (s, 2H), 4.64 (t, J = 7.1 Hz, 2H), 3.28 (t, J = 7.1 Hz, 2H), 2.67 (s, 3H), 1.65 (d, J = 6.7 Hz, 6H). | m/z: 535.1 [M + H]⁺ |
| 276 | 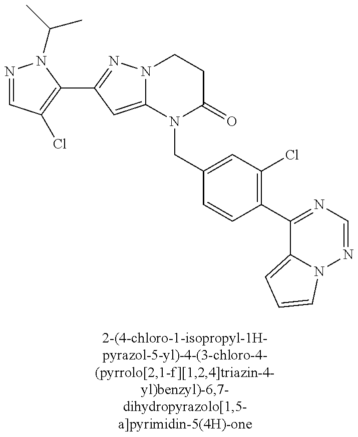 [SMILES: CC(C)n1ncc(Cl)c1-c1cc2n(n1)CCC(=O)N2Cc1ccc(-c2ncnn3cccc23)c(Cl)c1] | ¹H NMR (400 MHz, Chloroform-d) δ 8.51 (s, 1H), 7.89 (s, 1H), 7.54 (d, J = 7.9 Hz, 2H), 7.49 (s, 1H), 7.40 (s, 1H), 6.94 (d, J = 2.0 Hz, 1H), 6.64 (d, J = 4.6 Hz, 1H), 6.19 (s, 1H), 5.25-5.14 (m, 1H), 5.05 (s, 2H), 4.46 (t, J = 7.2 Hz, 2H), 3.13 (t, J = 7.0 Hz, 2H), 1.46 (d, J = 6.6 Hz, 6H). | m/z: 521.1 [M + H]⁺ |
| 277 | 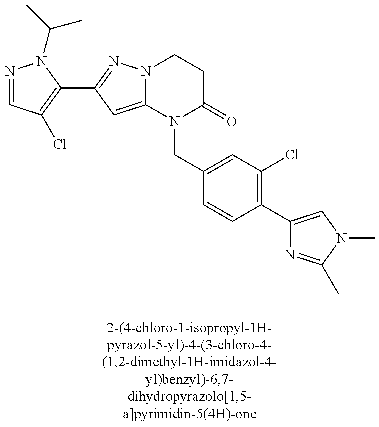 [SMILES: Cc1nc(-c2ccc(CN3C(=O)CCn4nc(-c5c(Cl)cnn5C(C)C)cc43)cc2Cl)cn1C] | ¹H NMR (400 MHz, Chloroform-d) δ 8.07 (d, J = 8.1 Hz, 1H), 7.49 (d, J = 10.7 Hz, 2H), 7.36 (d, J = 1.8 Hz, 1H), 7.27 (d, J = 1.9 Hz, 1H), 6.15 (s, 1H), 5.17-5.07 (m, 1H), 4.95 (s, 2H), 4.42 (t, J = 7.1 Hz, 2H), 3.61 (s, 3H), 3.09 (t, J = 7.1 Hz, 2H), 2.43 (s, 3H), 1.43 (d, J = 6.6 Hz, 6H). | m/z: 498.1 [M + H]⁺ |
| 278 | 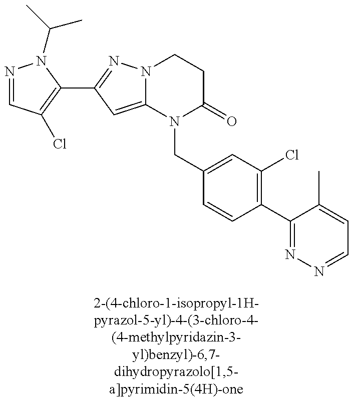 [SMILES: Cc1ccnnc1-c1ccc(CN2C(=O)CCn3nc(-c4c(Cl)cnn4C(C)C)cc32)cc1Cl] | ¹H NMR (400 MHz, Chloroform-d) δ 9.10 (s, 1H), 7.49 (d, J = 2.5 Hz, 2H), 7.39 (d, J = 8.6 Hz, 3H), 6.19 (s, 1H), 5.22-5.11 (m, 1H), 4.04 (s, 2H), 4.45 (t, J = 6.9 Hz, 2H), 3.12 (t, J = 6.9 Hz, 2H), 2.20 (s, 3H), 1.46 (d, J = 6.6 Hz, 6H). | m/z: 496.1 [M + H]⁺ |
| 279 | 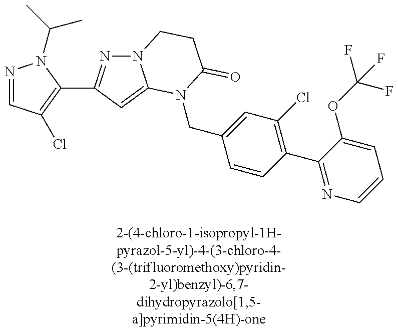 [SMILES: CC(C)n1ncc(Cl)c1-c1cc2n(n1)CCC(=O)N2Cc1ccc(-c2ncccc2OC(F)(F)F)c(Cl)c1] | ¹H NMR (400 MHz, Chloroform-d) δ 8.61 (dd, J = 4.7, 1.1 Hz, 1H), 7.69 (d, J = 8.4 Hz, 1H), 7.48 (d, J = 7.4 Hz, 2H), 7.41 (dd, J = 8.4, 4.7 Hz, 1H), 7.36 (d, J = 2.9 Hz, 2H), 6.17 (s, 1H), 5.15 (m, 1H), 5.02 (s, 2H), 4.44 (t, J = 7.1 Hz, 2H), 3.11 (t, J = 7.1 Hz, 2H), 1.45 (d, J = 6.6 Hz, 6H). | m/z: 565.1 [M + H]⁺ |
| 280 | 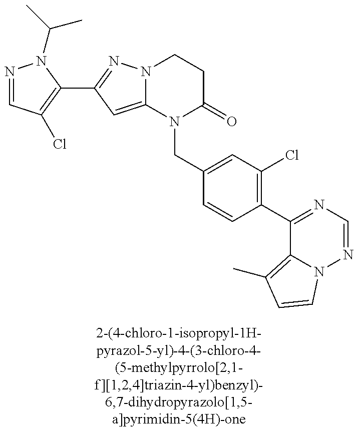 [SMILES: Cc1ccn2ncnc(-c3ccc(CN4C(=O)CCn5nc(-c6c(Cl)cnn6C(C)C)cc54)cc3Cl)c12] | ¹H NMR (400 MHz, Chloroform-d) δ 8.36 (s, 1H), 7.80 (d, J = 2.5 Hz, 1H), 7.49 (s, 2H), 7.38 (s, 2H), 6.71 (d, J = 2.1 Hz, 1H), 6.15 (s, 1H), 5.18 (m, 1H), 5.06 (d, J = 1.6 Hz, 2H), 4.46 (t, J = 7.1 Hz, 2H), 3.13 (t, J = 7.1 Hz, 2H), 1.86 (s, 3H), 1.48-1.44 (m, 6H). | m/z: 535.2 [M + H]⁺ |
| 281 | 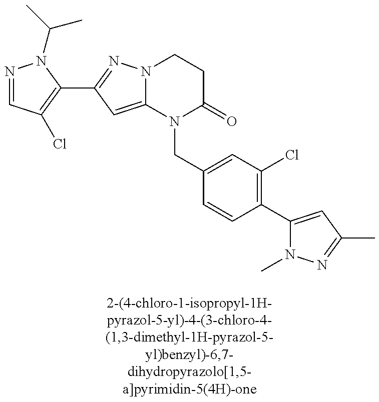 [SMILES: Cc1cc(-c2ccc(CN3C(=O)CCn4nc(-c5c(Cl)cnn5C(C)C)cc43)cc2Cl)n(C)n1] | ¹H NMR (400 MHz, Chloroform-d) δ 7.49 (s, 1H), 7.46 (s, 1H), 7.29 (s, 2H), 6.17 (s, 1H), 6.03 (s, 1H), 5.21 (m, 1H), 5.01 (s, 2H), 4.46 (t, J = 7.0 Hz, 2H), 3.63 (s, 3H), 3.12 (t, J = 7.0 Hz, 2H), 2.30 (s, 3H), 1.45 (d, J = 6.6 Hz, 6H). | m/z: 498.1 [M + H]⁺ |
| 282 | 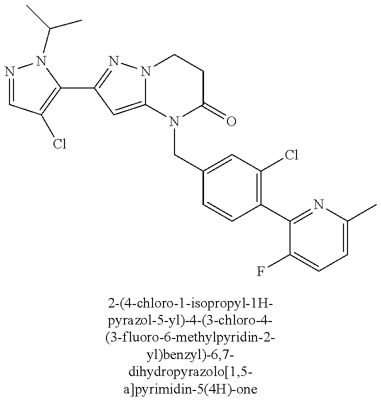 [SMILES: Cc1ccc(F)c(-c2ccc(CN3C(=O)CCn4nc(-c5c(Cl)cnn5C(C)C)cc43)cc2Cl)n1] | ¹H NMR (400 MHz, Chloroform-d) δ 7.50 (s, 1H), 7.47 (d, J = 1.4 Hz, 1H), 7.42 (d, J = 7.9 Hz, 1H), 7.41-7.34 (m, 2H), 7.19 (dd, J = 8.4, 3.6 Hz, 1H), 6.18 (s, 1H), 5.15 (m, 1H), 5.01 (s, 2H), 4.43 (t, J = 7.1 Hz, 2H), 3.10 (t, J = 7.1 Hz, 2H), 2.57 (s, 3H), 1.46 (d, J = 6.6 Hz, 6H). | m/z: 513.1 [M + H]⁺ |
| 283 | 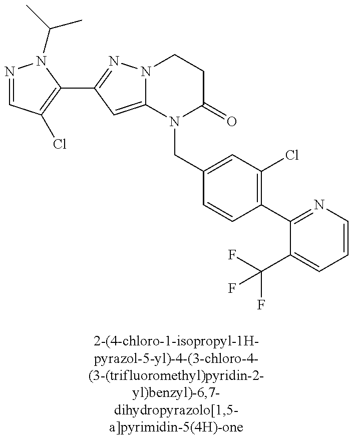 [SMILES: CC(C)n1ncc(Cl)c1-c1cc2n(n1)CCC(=O)N2Cc1ccc(-c2ncccc2C(F)(F)F)c(Cl)c1] | ¹H NMR (400 MHz, Chloroform-d) δ 8.87-8.81 (m, 1H), 8.12-8.06 (m, 1H), 7.52-7.46 (m, 3H), 7.35-7.27 (m, 2H), 6.17 (s, 1H), 5.14 (m, 1H), 5.08-4.97 (m, 2H), 4.45 (t, J = 7.1 Hz, 2H), 3.15- 3.08 (m, 2H), 1.46 (d, J = 8.0 Hz, 6H). | m/z: 549.1 [M + H]⁺ |
| 284 | 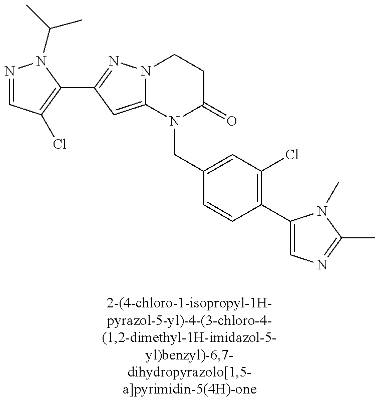 [SMILES: Cc1ncc(-c2ccc(CN3C(=O)CCn4nc(-c5c(Cl)cnn5C(C)C)cc43)cc2Cl)n1C] | ¹H NMR (400 MHz, Methanol-d₄) δ 7.62 (d, J = 1.7 Hz, 1H), 7.50 (s, 1H), 7.43 (dd, J = 7.9, 1.8 Hz, 1H), 7.38 (d, J = 7.9 Hz, 1H), 6.97 (s, 1H), 6.26 (s, 1H), 5.19-5.09 (m, 3H), 4.47 (t, J = 7.1 Hz, 2H), 3.40 (s, 3H), 3.15 (t, J = 7.1 Hz, 2H), 2.47 (s, 3H), 1.40 (d, J = 6.6 Hz, 6H). | m/z: 498.1 [M + H]⁺ |
| 285 | 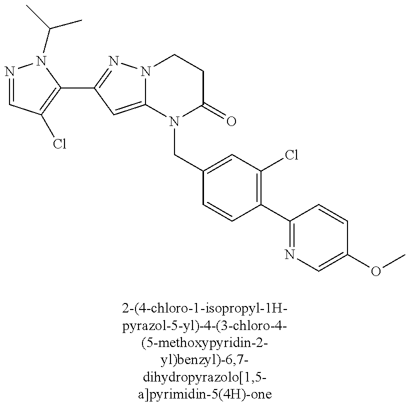 [SMILES: COc1ccc(-c2ccc(CN3C(=O)CCn4nc(-c5c(Cl)cnn5C(C)C)cc43)cc2Cl)nc1] | ¹H NMR (400 MHz, DMSO- d₆) δ 8.38 (d, J = 2.9 Hz, 1H), 7.64 (s, 1H), 7.62-7.56 (m, 2H), 7.54-7.45 (m, 2H), 7.41 (dd, J = 8.0, 1.7 Hz, 1H), 6.38 (s, 1H), 5.12-4.02 (m, 3H), 4.44 (t, J = 7.1 Hz, 2H), 3.87 (s, 3H), 3.10 (t, J = 7.1 Hz, 2H), 1.35 (d, J = 6.6 Hz, 6H). | m/z: 511.1 [M + H]⁺ |
| 286 | 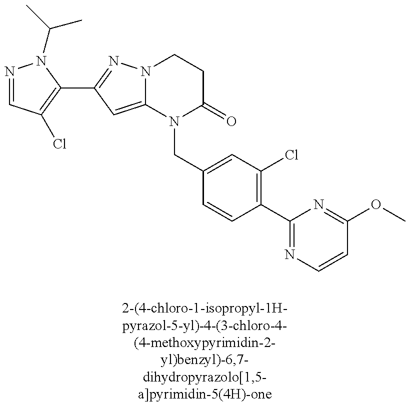 [SMILES: COc1ccnc(-c2ccc(CN3C(=O)CCn4nc(-c5c(Cl)cnn5C(C)C)cc43)cc2Cl)n1] | ¹H NMR (400 MHz, Chloroform-d) δ 8.53 (d, J = 5.8 Hz, 1H), 7.78 (d, J = 7.9 Hz, 1H), 7.54-7.44 (m, 2H), 7.34 (dd, J = 8.0, 1.7 Hz, 1H), 6.70 (d, J = 5.8 Hz, 1H), 6.17 (s, 1H), 5.16 (m, 1H), 5.01 (s, 2H), 4.44 (t, J = 7.1 Hz, 2H), 4.01 (s, 3H), 3.11 (t, J = 7.1 Hz, 2H), 1.45 (d, J = 6.6 Hz, 6H). | m/z: 512.1 [M + H]⁺ |
| 287 | 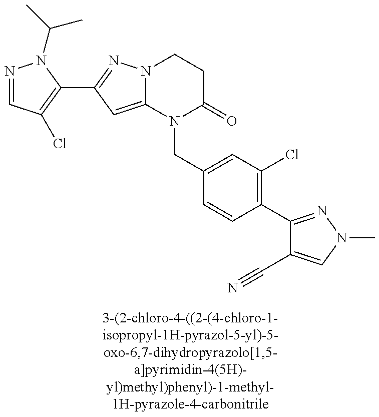 [SMILES: CC(C)n1ncc(Cl)c1-c1cc2n(n1)CCC(=O)N2Cc1ccc(-c2nn(C)cc2C#N)c(Cl)c1] | ¹H NMR (400 MHz, Chloroform-d) δ 7.86 (s, 1H), 7.50-7.48 (m, 2H), 7.45 (d, J = 7.9 Hz, 1H), 7.32 (dd, J = 7.9, 1.7 Hz, 1H), 6.17 (s, 1H), 5.21-5.12 (m, 1H), 5.00 (s, 2H), 4.44 (t, J = 7.1 Hz, 2H), 3.98 (s, 3H), 3.10 (t, J = 7.1 Hz, 2H), 1.45 (d, J = 6.6 Hz, 6H). | m/z: 509.1 [M + H]⁺ |
| 288 | 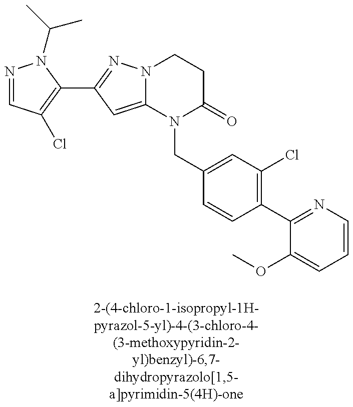 [SMILES: COc1cccnc1-c1ccc(CN2C(=O)CCn3nc(-c4c(Cl)cnn4C(C)C)cc32)cc1Cl] | ¹H NMR (400 MHz, Chloroform-d) δ 8.26 (dd, J = 4.3, 1.7 Hz, 1H), 7.49 (s, 1H), 7.44 (d, J = 1.7 Hz, 1H), 7.37 (d, J = 7.8 Hz, 1H), 7.34-7.28 (m, 3H), 6.19 (s, 1H), 5.22- 5.11 (m, 1H), 5.00 (s, 2H), 4.42 (t, J = 7.1 Hz, 2H), 3.80 (s, 3H), 3.09 (t, J = 7.1 Hz, 2H), 1.45 (d, J = 6.6 Hz, 6H). | m/z: 511.3 [M + H]⁺ |
| 289 | 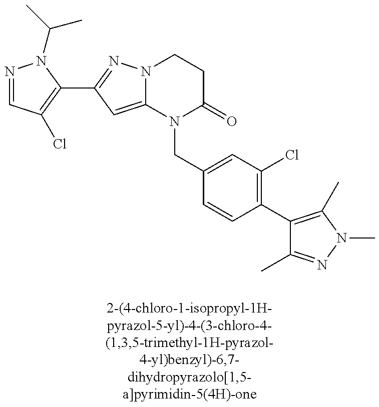 [SMILES: Cc1nn(C)c(C)c1-c1ccc(CN2C(=O)CCn3nc(-c4c(Cl)cnn4C(C)C)cc32)cc1Cl] | ¹H NMR (400 MHz, Chloroform-d) δ 7.49 (s, 1H), 7.44 (d, J = 1.8 Hz, 1H), 7.24 (dd, J = 7.9, 1.8 Hz, 1H), 7.16 (d, J = 7.8 Hz, 1H), 6.18 (s, 1H), 5.19 (m, 1H), 4.99 (s, 2H), 4.45 (t, J = 7.1 Hz, 2H), 3.77 (s, 3H), 3.12 (t, J = 7.1 Hz, 2H), 2.08 (d, J = 5.4 Hz, 6H), 1.45 (d, J = 6.6 Hz, 6H). | m/z: 512.1 [M + H]⁺ |
| 290 | 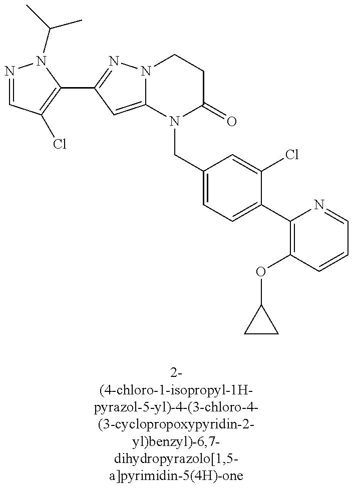 [SMILES: CC(C)n1ncc(Cl)c1-c1cc2n(n1)CCC(=O)N2Cc1ccc(-c2ncccc2OC2CC2)c(Cl)c1] | ¹H NMR (400 MHz, Chloroform-d) δ 8.30 (s, 1H), 7.68 (d, J = 8.2 Hz, 1H), 7.49 (s, 1H), 7.42 (s, 1H), 7.35 (d, J = 10.6 Hz, 3H), 6.19 (s, 1H), 5.16 (m, 1H), 5.00 (s, 2H), 4.44 (t, J = 7.0 Hz, 2H), 3.75 (d, J = 3.2 Hz, 1H), 3.10 (t, J = 7.0 Hz, 2H), 1.46 (d, J = 6.6 Hz, 6H), 0.76 (s, 4H). | m/z: 537.2 [M + H]⁺ |
| 291 | 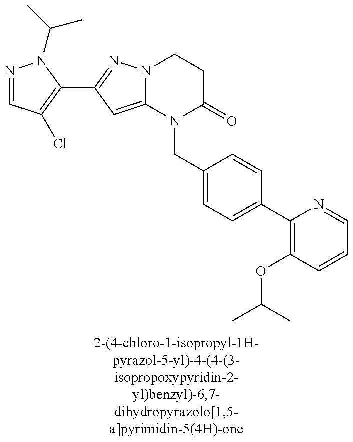 [SMILES: CC(C)Oc1cccnc1-c1ccc(CN2C(=O)CCn3nc(-c4c(Cl)cnn4C(C)C)cc32)cc1] | ¹H NMR (400 MHz, Chloroform-d) δ 8.24 (dd, J = 4.6, 1.3 Hz, 1H), 7.93-7.89 (m, 2H), 7.45 (s, 1H), 7.39- 7.35 (m, 2H), 7.24 (s, 1H), 7.16 (dd, J = 8.3, 4.5 Hz, 1H), 6.16 (s, 1H), 5.12 (m, 1H), 5.02 (s, 2H), 4.50 (m, 1H), 4.40 (t, J = 7.1 Hz, 2H), 3.07 (t, J = 7.1 Hz, 2H), 1.41 (d, J = 6.6 Hz, 6H), 1.29 (d, J = 6.0 Hz, 6H). | m/z: 505.2 [M + H]⁺ |
| 292 | 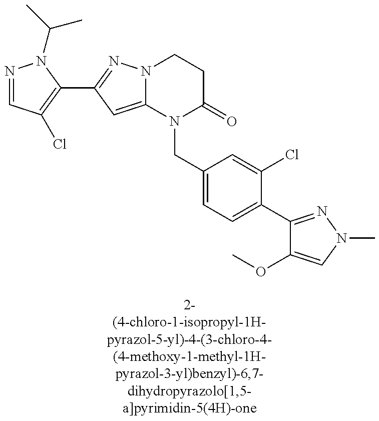 [SMILES: COc1cn(C)nc1-c1ccc(CN2C(=O)CCn3nc(-c4c(Cl)cnn4C(C)C)cc32)cc1Cl] | ¹H NMR (400 MHz, Chloroform-d) δ 7.48 (s, 1H), 7.45 (d, J = 8.0 Hz, 2H), 7.27 (d, J = 1.5 Hz, 1H), 7.10 (s, 1H), 6.16 (s, 1H), 5.14 (m, 1H), 4.97 (s, 2H), 4.42 (t, J = 7.1 Hz, 2H), 3.87 (s, 3H), 3.72 (s, 3H), 3.08 (t, J = 7.1 Hz, 2H), 1.45 (d, J = 6.6 Hz, 6H). | m/z: 514.1 [M + H]⁺ |
| 293 | 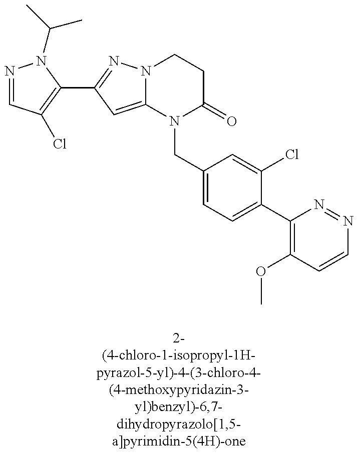 [SMILES: COc1ccnnc1-c1ccc(CN2C(=O)CCn3nc(-c4c(Cl)cnn4C(C)C)cc32)cc1Cl] | ¹H NMR (400 MHz, Chloroform-d) δ 9.15 (s, 1H), 7.48 (d, J = 4.5 Hz, 2H), 7.44 (d, J = 7.8 Hz, 1H), 7.38 (d, J = 7.7 Hz, 1H), 7.12 (s, 1H), 6.19 (s, 1H), 5.18 (m, 1H), 5.03 (s, 2H), 4.45 (t, J = 7.0 Hz, 2H), 3.93 (s, 3H), 3.11 (t, J = 7.1 Hz, 2H), 1.46 (d, J = 6.6 Hz, 6H). | m/z: 512.1 [M + H]⁺ |
| 294 | 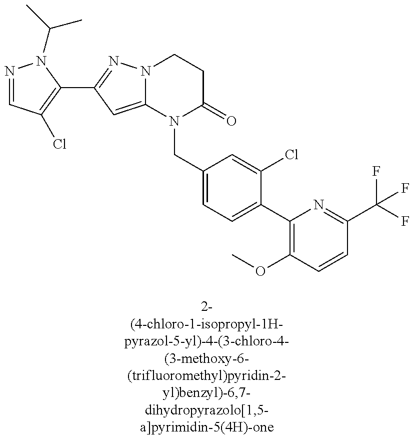 [SMILES: COc1ccc(C(F)(F)F)nc1-c1ccc(CN2C(=O)CCn3nc(-c4c(Cl)cnn4C(C)C)cc32)cc1Cl] | ¹H NMR (400 MHz, Chloroform-d) δ 7.70 (d, J = 8.6 Hz, 1H), 7.49 (s, 1H), 7.45 (d, J = 1.2 Hz, 1H), 7.40-7.32 (m, 3H), 6.19 (s, 1H), 5.16 (m, 1H), 5.00 (s, 2H), 4.43 (t, J = 7.0 Hz, 2H), 3.86 (s, 3H), 3.09 (t, J = 7.0 Hz, 2H), 1.46 (d, J = 6.6 Hz, 6H). | m/z: 579.1 [M + H]⁺ |
| 295 | 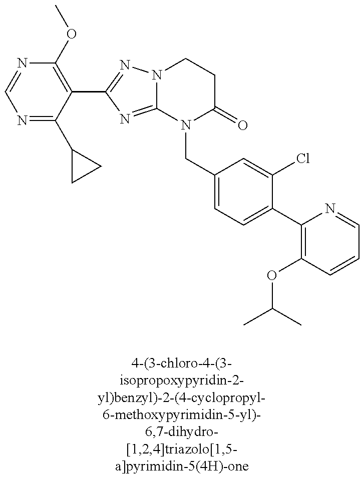 [SMILES: COc1ncnc(C2CC2)c1-c1nc2n(n1)CCC(=O)N2Cc1ccc(-c2ncccc2OC(C)C)c(Cl)c1] | ¹H NMR (400 MHz, Chloroform-d) δ 8.62 (s, 1H), 8.23 (t, J = 3.0 Hz, 7.64 (d, J = 1.5 Hz, 1H), 7.52 (dd, J = 7.9, 1.5 Hz, 1H), 7.31 (d, J = 7.8 Hz, 1H), 7.27 (d, J = 3.0 Hz, 2H), 5.18 (s, 2H), 4.48 (m, 1H), 4.41 (t, J = 7.3 Hz, 2H), 3.97 (s, 3H), 3.12 (t, J = 7.3 Hz, 2H), 2.05 (tt, J = 8.1, 4.6 Hz, 1H), 1.25 (d, J = 6.1 Hz, 6H), 1.21 (dd, J = 4.5, 2.9 Hz, 2H), 1.00 (dd, J = 8.0, 3.0 Hz, 2H). | m/z: 546.2 [M + H]⁺ |
| 296 | 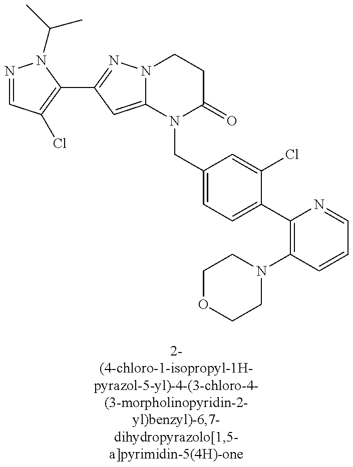 [SMILES: CC(C)n1ncc(Cl)c1-c1cc2n(n1)CCC(=O)N2Cc1ccc(-c2ncccc2N2CCOCC2)c(Cl)c1] | ¹H NMR (400 MHz, Chloroform-d) δ 8.35 (d, J = 4.3 Hz, 1H), 7.49-7.38 (m, 4H), 7.31 (dd, J = 13.5, 8.0 Hz, 2H), 6.16 (s, 1H), 5.18 (m, 1H), 5.01 (s, 2H), 4.43 (t, J = 7.0 Hz, 2H), 3.47 (t, J = 4.4 Hz, 4H), 3.10 (t, J = 7.0 Hz, 2H), 2.79 (t, J = 4.5 Hz, 4H), 1.45 (d, J = 6.6 Hz, 6H). | m/z: 566.2 [M + H]⁺ |
| 297 | 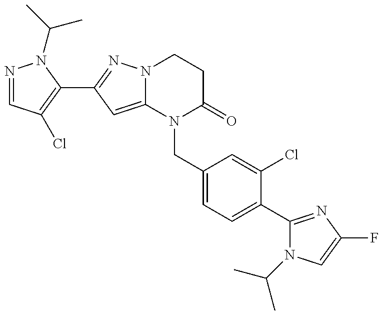 [SMILES: CC(C)n1cc(F)nc1-c1ccc(CN2C(=O)CCn3nc(-c4c(Cl)cnn4C(C)C)cc32)cc1Cl] | ¹H NMR (400 MHz, Chloroform-d) δ 7.49 (s, 1H), 7.45 (s, 1H), 7.38 (d, J = 7.7 Hz, 1H), 7.31 (d, J = 7.2 Hz, 1H), 6.55 (d, J = 8.0 Hz, 1H), 6.15 (s, 1H), 5.18 (m, 1H), 5.01 (s, 2H), 4.45 (t, J = 7.1 Hz, 2H), 4.04 (m, 1H), 3.12 (t, J = 7.1 Hz, 2H), 1.46 (d, J = 6.6 Hz, 6H), 1.34 (d, J = 6.6 Hz, 6H). | m/z: 530.2 [M + H]⁺ |
| 298 | 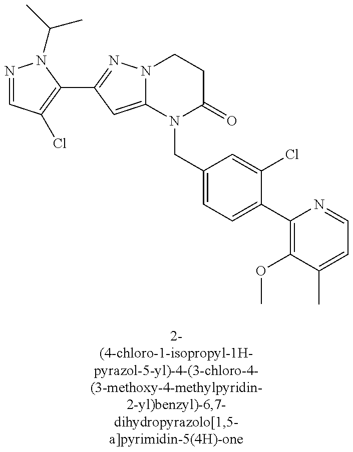 [SMILES: COc1c(C)ccnc1-c1ccc(CN2C(=O)CCn3nc(-c4c(Cl)cnn4C(C)C)cc32)cc1Cl] | ¹H NMR (400 MHz, Chloroform-d) δ 8.28 (d, J = 4.9 Hz, 1H), 7.51-7.43 (m, 2H), 7.39 (d, J = 7.8 Hz, 1H), 7.31 (dd, J = 7.8, 1.7 Hz, 1H), 7.16 (dd, J = 4.9, 0.8 Hz, 1H), 6.17 (s, 1H), 5.17 (m, 1H), 5.02 (s, 2H), 4.44 (t, J = 7.1 Hz, 2H), 3.43 (s, 3H), 3.11 (t, J = 7.1 Hz, 2H), 2.36 (d, J = 0.6 Hz, 3H), 1.46 (d, J = 6.6 Hz, 6H). | m/z: 525.1 [M + H]⁺ |
| 299 | 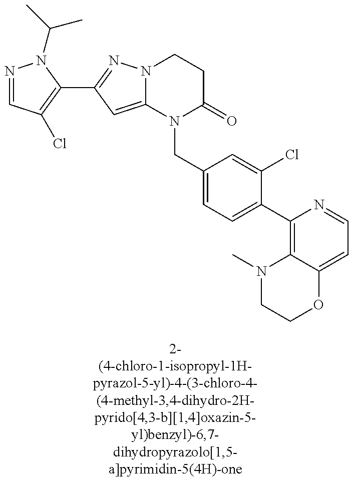 [SMILES: CC(C)n1ncc(Cl)c1-c1cc2n(n1)CCC(=O)N2Cc1ccc(-c2nccc3c2N(C)CCO3)c(Cl)c1] | ¹H NMR (400 MHz, Chloroform-d) δ 8.13 (s, 1H), 7.54-7.46 (m, 3H), 7.36 (s, 1H), 6.88 (s, 1H), 6.16 (s, 1H), 5.18 (m, 1H), 5.02 (s, 2H), 4.45 (t, J = 6.8 Hz, 2H), 4.28 (s, 2H), 3.21 (s, 2H), 3.11 (t, J = 6.8 Hz, 2H), 2.34 (s, 3H), 1.46 (d, J = 6.5 Hz, 6H). | m/z: 552.1 [M + H]⁺ |
| 300 | 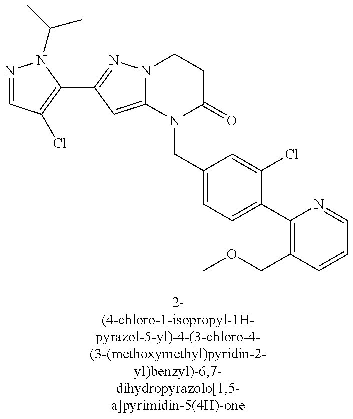 [SMILES: COCc1cccnc1-c1ccc(CN2C(=O)CCn3nc(-c4c(Cl)cnn4C(C)C)cc32)cc1Cl] | ¹H NMR (400 MHz, Chloroform-d) δ 8.58 (dd, J = 4.8, 1.7 Hz, 1H), 7.89 (m, 1H), 7.47 (d, J = 13.6 Hz, 2H), 7.37-7.29 (m, 3H), 6.18 (s, 1H), 5.18 (m, 1H), 5.02 (s, 2H), 4.44 (t, J = 7.1 Hz, 2H), 4.24 (d, J = 47.5 Hz, 2H), 3.27 (s, 3H), 3.11 (t, J = 7.1 Hz, 2H), 1.46 (d, J = 6.6 Hz, 6H). | m/z: 525.1 [M + H]⁺ |
| 301 | 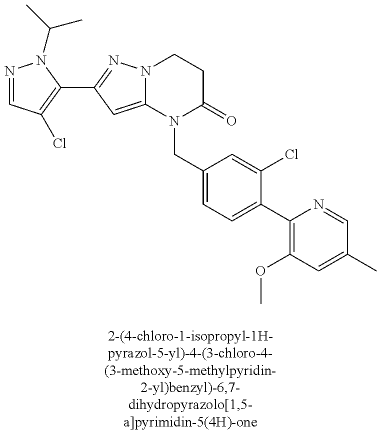 [SMILES: COc1cc(C)cnc1-c1ccc(CN2C(=O)CCn3nc(-c4c(Cl)cnn4C(C)C)cc32)cc1Cl] | ¹H NMR (400 MHz, Chloroform-d) δ 8.11 (s, 1H), 7.49 (s, 1H), 7.44 (s, 1H), 7.36 (d, J = 7.8 Hz, 1H), 7.31 (d, J = 7.5 Hz, 1H), 7.10 (s, 1H), 6.19 (s, 1H), 5.16 (m, 1H), 5.00 (s, 2H), 4.43 (t, J = 7.1 Hz, 2H), 3.79 (s, 3H), 3.09 (t, J = 7.1 Hz, 2H), 2.40 (s, 3H), 1.46 (d, J = 6.6 Hz, 6H). | m/z: 525.1 [M + H]⁺ |
| 302 | 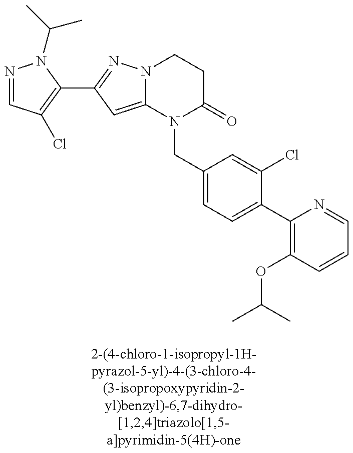 [SMILES: CC(C)Oc1cccnc1-c1ccc(CN2C(=O)CCn3nc(-c4c(Cl)cnn4C(C)C)cc32)cc1Cl] | ¹H NMR (400 MHz, Chloroform-d) δ 8.24 (t, J = 3.0 Hz, 1H), 7.68 (d, J = 1.6 Hz, 1H), 7.53 (d, J = 8.3 Hz, 2H), 7.34 (d, J = 7.9 Hz, 1H), 7.27 (d, J = 3.0 Hz, 2H), 5.27- 5.16 (m, 3H), 4.52-4.39 (m, 3H), 3.12 (t, J = 7.3 Hz, 2H), 1.52 (d, J = 6.6 Hz, 6H), 1.24 (d, J = 6.0 Hz, 6H). | m/z: 540.1 [M + H]⁺ |
| 303 | 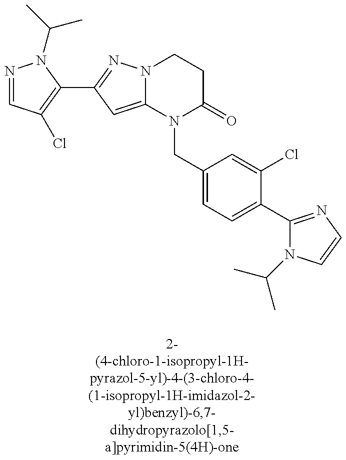 [SMILES: CC(C)n1ccnc1-c1ccc(CN2C(=O)CCn3nc(-c4c(Cl)cnn4C(C)C)cc32)cc1Cl] | ¹H NMR (400 MHz, Chloroform-d) δ 7.60-7.47 (m, 5H), 7.29 (s, 1H), 6.21 (s, 1H), 5.20 (m, 1H), 5.03 (s, 2H), 4.48 (t, J = 7.1 Hz, 2H), 4.25 (m, 1H), 3.12 (t, J = 7.1 Hz, 2H), 1.46 (d, J = 6.6 Hz, 12H). | m/z: 512.2 [M + H]⁺ |
| 304 | 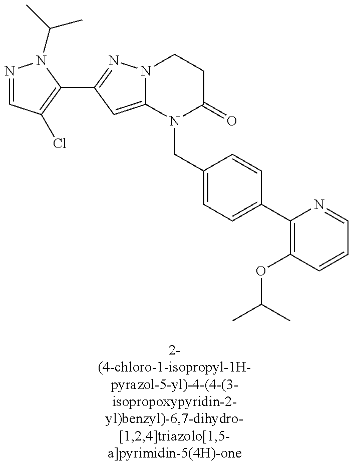 [SMILES: CC(C)Oc1cccnc1-c1ccc(CN2C(=O)CCn3nc(-c4c(Cl)cnn4C(C)C)cc32)cc1] | ¹H NMR (400 MHz, Chloroform-d) δ 8.28 (d, J = 4.5 Hz, 1H), 7.94 (d, J = 8.2 Hz, 2H), 7.60 (d, J = 8.2 Hz, 2H), 7.52 (s, 1H), 7.28 (d, J = 8.3 Hz, 1H), 7.18 (dd, J = 8.3, 4.5 Hz, 1H), 5.22 (d, J = 11.7 Hz, 3H), 4.54 (m, 1H), 4.39 (t, J = 7.4 Hz, 2H), 3.11 (t, J = 7.4 Hz, 2H), 1.50 (d, J = 6.6 Hz, 6H), 1.32 (d, J = 6.0 Hz, 6H). | m/z: 506.1 [M + H]⁺ |
| 305 | 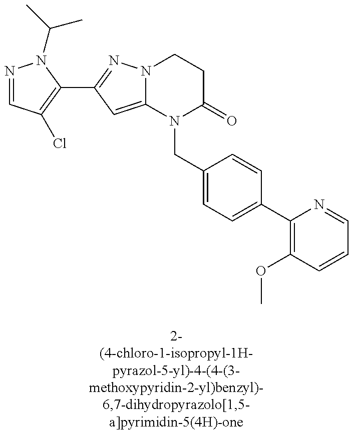 [SMILES: COc1cccnc1-c1ccc(CN2C(=O)CCn3nc(-c4c(Cl)cnn4C(C)C)cc32)cc1] | ¹H NMR (400 MHz, Chloroform-d) δ 8.29 (d, J = 3.5 Hz, 1H), 7.87 (d, J = 8.2 Hz, 2H), 7.48 (s, 1H), 7.41 (d, J = 8.3 Hz, 2H), 7.28 (dd, J = 8.3, 1.2 Hz, 1H), 7.23 (dd, J = 8.3, 4.6 Hz, 1H), 6.19 (s, 1H), 5.14 (m, 1H), 5.04 (s, 2H), 4.42 (t, J = 7.1 Hz, 2H), 3.85 (s, 3H), 3.09 (t, J = 7.1 Hz, 2H), 1.44 (d, J = 6.6 Hz, 6H). | m/z: 477.2 [M + H]⁺ |
| 306 | 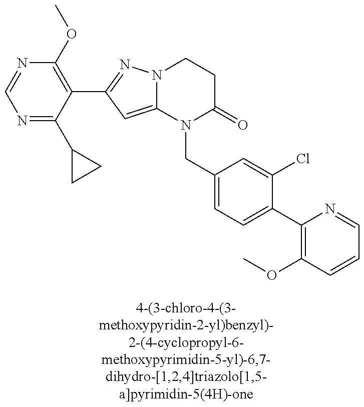 [SMILES: COc1cccnc1-c1ccc(CN2C(=O)CCn3nc(-c4c(OC)ncnc4C4CC4)cc32)cc1Cl] | ¹H NMR (400 MHz, Chloroform-d) δ 8.62 (s, 1H), 8.27 (d, J = 3.2 Hz, 1H), 7.67 (s, 1H), 7.56 (d, J = 7.4 Hz, 1H), 7.36-7.29 (m, 3H), 5.17 (s, 2H), 4.41 (t, J = 7.3 Hz, 2H), 3.97 (s, 3H), 3.81 (s, 3H), 3.12 (t, J = 7.3 Hz, 2H), 2.04 (tt, J = 8.3, 4.7 Hz, 1H), 1.24-1.19 (m, 2H), 0.99 (dq, J = 7.0, 3.8 Hz, 2H). | m/z: 518.1 [M + H]⁺ |
| 307 | 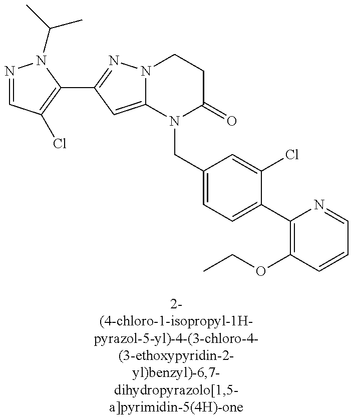 [SMILES: CCOc1cccnc1-c1ccc(CN2C(=O)CCn3nc(-c4c(Cl)cnn4C(C)C)cc32)cc1Cl] | ¹H NMR (400 MHz, Chloroform-d) δ 8.25 (s, 1H), 7.49 (s, 1H), 7.43 (s, 1H), 7.39 (d, J = 7.8 Hz, 1H), 7.32 (d, J = 8.4 Hz, 1H), 7.28 (s, 2H), 6.18 (s, 1H), 5.16 (m, 1H), 5.01 (s, 2H), 4.44 (t, J = 7.1 Hz, 2H), 4.05 (q, J = 7.0 Hz, 2H), 3.10 (t, J = 7.1 Hz, 2H), 1.46 (d, J = 6.6 Hz, 6H), 1.30 (t, J = 7.0 Hz, 3H). | m/z: 525.0 [M + H]⁺ |
| 308 | 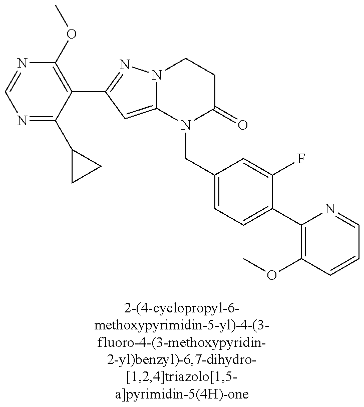 [SMILES: COc1cccnc1-c1ccc(CN2C(=O)CCn3nc(-c4c(OC)ncnc4C4CC4)cc32)cc1F] | ¹H NMR (400 MHz, Chloroform-d) δ 8.62 (s, 1H), 8.29 (t, J = 3.0 Hz, 1H), 7.44 (d, J = 4.7 Hz, 2H), 7.39 (d, J = 11.0 Hz, 1H), 7.29 (d, J = 3.1 Hz, 2H), 5.19 (s, 2H), 4.40 (t, J = 7.3 Hz, 2H), 3.97 (s, 3H), 3.83 (s, 3H), 3.12 (t, J = 7.4 Hz, 2H), 2.05 (tt, J = 8.3, 4.6 Hz, 1H), 1.21 (dt, J = 7.9, 3.9 Hz, 2H), 0.98 (dq, J = 6.9, 3.8 Hz, 2H). | m/z: 502.2 [M + H]⁺ |
| 309 | 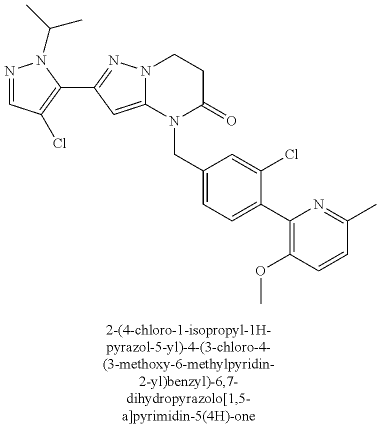 [SMILES: COc1ccc(C)nc1-c1ccc(CN2C(=O)CCn3nc(-c4c(Cl)cnn4C(C)C)cc32)cc1Cl] | ¹H NMR (400 MHz, Chloroform-d) δ 7.46 (d, J = 20.0 Hz, 2H), 7.38-7.29 (m, 2H), 7.22-7.10 (m, 2H), 6.20 (s, 1H), 5.22-5.11 (m, 1H), 4.99 (s, 2H), 4.42 (t, J = 6.4 Hz, 2H), 3.76 (s, 3H), 3.08 (t, J = 6.6 Hz, 2H), 2.51 (s, 3H), 1.46 (d, J = 6.2 Hz, 6H). | m/z: 525.1 [M + H]⁺ |
| 310 | 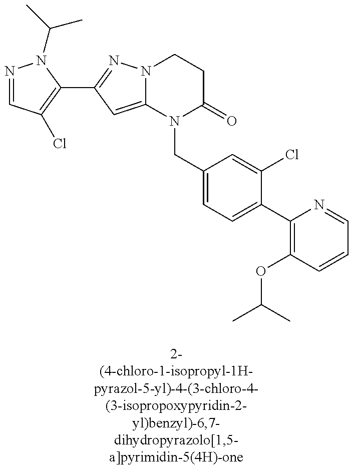 [SMILES: CC(C)Oc1cccnc1-c1ccc(CN2C(=O)CCn3nc(-c4c(Cl)cnn4C(C)C)cc32)cc1Cl] | ¹H NMR (400 MHz, Chloroform-d) δ 8.26 (s, 1H), 7.50-7.29 (m, 6H), 6.17 (s, 1H), 5.23-5.11 (m, 1H), 5.01 (s, 2H), 4.44 (s, 3H), 3.10 (s, 2H), 1.45 (d, J = 6.3 Hz, 6H), 1.24 (s, 6H). | m/z: 539.1 [M + H]⁺ |
| 311 | 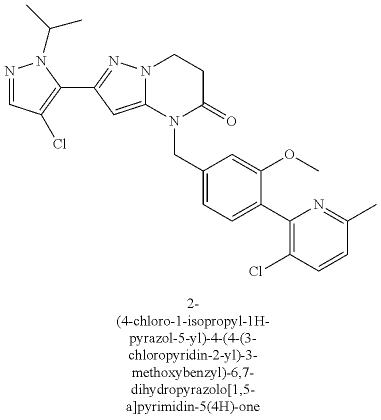 | ¹H NMR (400 MHz, Chloroform-d) δ 8.54 (dd, J = 4.7, 1.5 Hz, 1H), 7.75 (dd, J = 8.1, 1.5 Hz, 1H), 7.49 (s, 1H), 7.25-7.21 (m, 2H), 7.03 (dd, J = 7.7, 1.5 Hz, 1H), 7.00 (d, J = 1.5 Hz, 1H), 6.24 (s, 1H), 5.17 (m, 1H), 5.03 (s, 2H), 4.43 (t, J = 7.1 Hz, 2H), 3.78 (s, 3H), 3.09 (t, J = 7.1 Hz, 2H), 1.45 (d, J = 6.6 Hz, 6H). | m/z: 511.1 [M + H]⁺ |
| 312 | 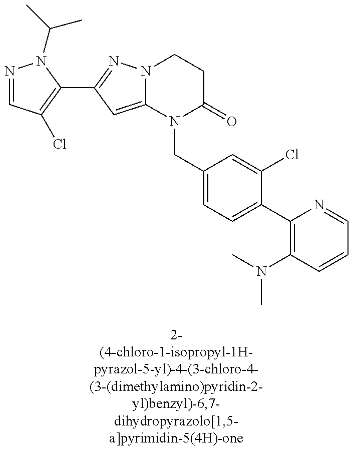 [SMILES: CC(C)n1ncc(Cl)c1-c1cc2n(n1)CCC(=O)N2Cc1ccc(-c2ncccc2N(C)C)c(Cl)c1] | ¹H NMR (400 MHz, Chloroform-d) δ 8.24 (s, 1H), 7.50-7.40 (m, 3H), 7.31 (dt, J = 16.9, 8.7 Hz, 2H), 7.22 (dd, J = 8.2, 4.5 Hz, 1H), 6.18- 6.12 (m, 1H), 5.21-5.12 (m, 1H), 5.01 (s, 2H), 4.43 (t, J = 6.9 Hz, 2H), 3.10 (t, J = 7.0 Hz, 2H), 2.57-2.53 (m, 6H), 1.45 (dd, J = 4.5, 2.0 Hz, 6H). | m/z: 524.1 [M + H]⁺ |
| 313 | 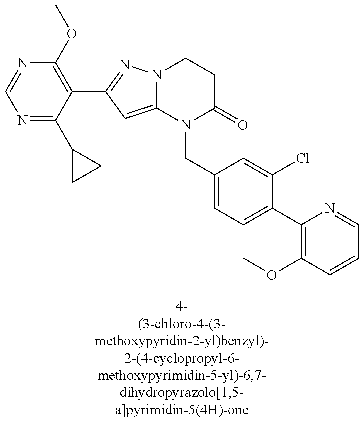 [SMILES: COc1cccnc1-c1ccc(CN2C(=O)CCn3nc(-c4c(OC)ncnc4C4CC4)cc32)cc1Cl] | ¹H NMR (400 MHz, Chloroform-d) δ 8.56 (s, 1H), 8.27 (d, J = 4.2 Hz, 1H), 7.43 (s, 1H), 7.37 (d, J = 7.8 Hz, 1H), 7.35-7.29 (m, 3H), 5.91 (s, H), 4.99 (s, 2H), 4.44 (t, J = 7.1 Hz, 2H), 3.93 (s, 3H), 3.81 (s, 3H), 3.10 (t, J = 7.1 Hz, 2H), 2.21 (tt, J = 8.3, 4.7 Hz, 1H), 1.16 (dt, J = 6.5, 3.2 Hz, 2H), 0.94 (dq, J = 6.9, 3.7 Hz, 2H). | m/z: 517.1 [M + H]⁺ |
| 314 | 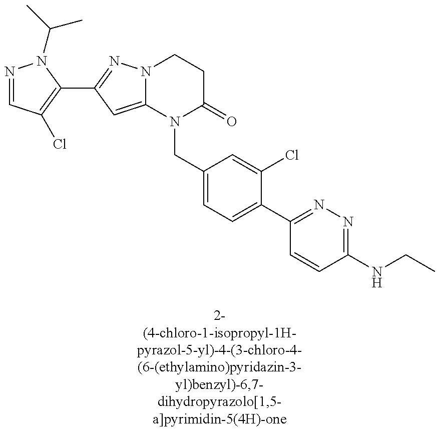 [SMILES: CCNc1ccc(-c2ccc(CN3C(=O)CCn4nc(-c5c(Cl)cnn5C(C)C)cc43)cc2Cl)nn1] | ¹H NMR (400 MHz, Chloroform-d) δ 7.66 (t, J = 8.4 Hz, 2H), 7.48 (s, 1H), 7.44 (d, J = 1.3 Hz, 1H), 7.34 (d, J = 7.0 Hz, 1H), 6.89 (s, 1H), 6.45 (s, 1H), 6.17 (s, 1H), 5.17 (m, 1H), 5.00 (s, 2H), 4.44 (t, J = 7.1 Hz, 2H), 3.47 (d, J = 6.0 Hz, 2H), 3.11 (t, J = 7.1 Hz, 2H), 1.47-1.43 (m, 6H), 1.34 (t, J = 6.9 Hz, 3H). | m/z: 525.2 [M + H]⁺ |
| 315 | 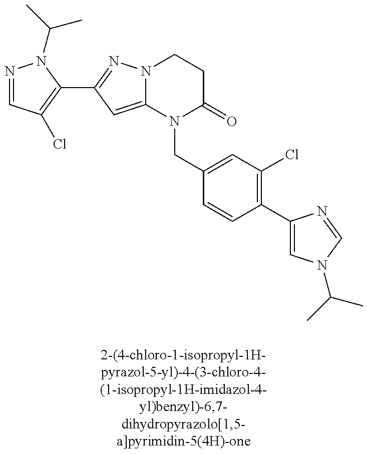 [SMILES: CC(C)n1cnc(-c2ccc(CN3C(=O)CCn4nc(-c5c(Cl)cnn5C(C)C)cc43)cc2Cl)c1] | ¹H NMR (400 MHz, Chloroform-d) δ 8.13 (d, J = 7.9 Hz, 1H), 7.70 (d, J = 18.4 Hz, 2H), 7.47 (s, 1H), 7.38 (s, 1H), 7.29 (d, J = 7.5 Hz, 1H), 6.15 (s, 1H), 5.14 (m, 1H), 4.96 (s, 2H), 4.42 (t, J = 7.0 Hz, 3H), 3.09 (t, J = 7.0 Hz, 2H), 1.54 (d, J = 5.9 Hz, 6H), 1.44 (d, J = 6.6 Hz, 6H). | m/z: 512.1 [M + H]⁺ |
| 316 | 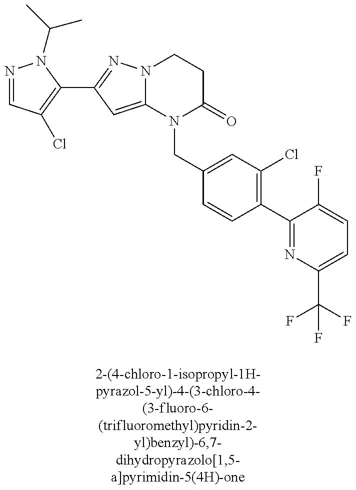 [SMILES: CC(C)n1ncc(Cl)c1-c1cc2n(n1)CCC(=O)N2Cc1ccc(-c2nc(C(F)(F)F)ccc2F)c(Cl)c1] | ¹H NMR (400 MHz, Chloroform-d) δ 7.77 (dd, J = 8.6, 3.5 Hz, 1H), 7.65 (td, J = 8.5, 0.8 Hz, 1H), 7.52-7.45 (m, 3H), 7.39 (dd, J = 7.9, 1.7 Hz, 1H), 6.18 (s, 1H), 5.16 (m, 1H), 5.02 (s, 2H), 4.45 (t, J = 7.1 Hz, 2H), 3.11 (t, J = 7.1 Hz, 2H), 1.46 (d, J = 6.6 Hz, 6H). | m/z: 567.2 [M + H]⁺ |
| 317 | 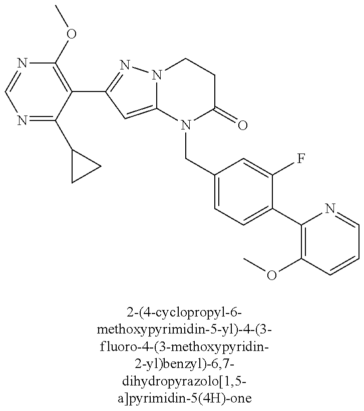 [SMILES: COc1cccnc1-c1ccc(CN2C(=O)CCn3nc(-c4c(OC)ncnc4C4CC4)cc32)cc1F] | ¹H NMR (400 MHz, Methanol-d₄) δ 8.52 (s, 1H), 8.20 (dd, J = 4.8, 1.3 Hz, 1H), 7.59 (dd, J = 8.5, 1.3 Hz, 1H), 7.49-7.41 (m, 2H), 7.31 (dd, J = 7.9, 1.7 Hz, 1H), 7.23 (dd, J = 10.6, 1.7 Hz, 1H), 6.07 (s, 1H), 5.13 (s, 2H), 4.47 (t, J = 7.1 Hz, 2H), 3.92 (s, 3H), 3.85 (s, 3H), 3.17 (t, J = 7.1 Hz, 2H), 2.18 (tt, J = 8.1, 4.7 Hz, 1H), 1.15-1.10 (m, 2H), 0.96- 0.91 (m, 2H). | m/z: 501.2 [M + H]⁺ |
| 318 | 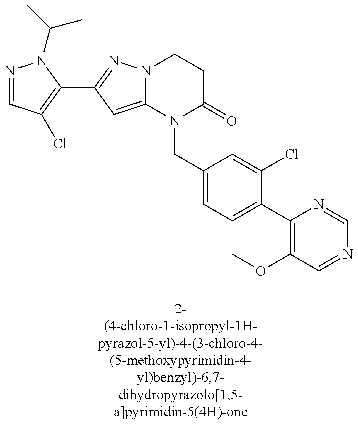 [SMILES: COc1cncnc1-c1ccc(CN2C(=O)CCn3nc(-c4c(Cl)cnn4C(C)C)cc32)cc1Cl] | ¹H NMR (400 MHz, Chloroform-d) δ 8.91 (s, 1H), 8.47 (s, 1H), 7.48 (s, 1H), 7.46 (d, J = 1.1 Hz, 1H), 7.39- 7.33 (m, 2H), 6.18 (s, 1H), 5.17 (m, 1H), 5.01 (s, 2H), 4.43 (t, J = 7.1 Hz, 2H), 3.90 (s, 3H), 3.10 (t, J = 7.1 Hz, 2H), 1.46 (s, 3H), 1.44 (s, 3H). | m/z: 512.1 [M + H]⁺ |
| 319 | 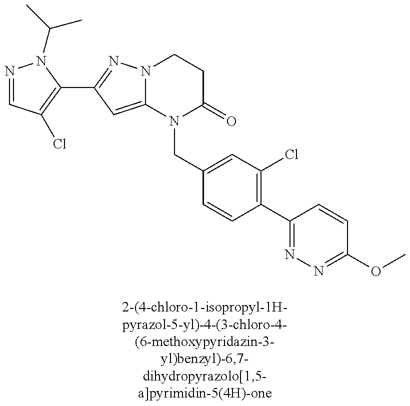 [SMILES: COc1ccc(-c2ccc(CN3C(=O)CCn4nc(-c5c(Cl)cnn5C(C)C)cc43)cc2Cl)nn1] | ¹H NMR (400 MHz, Chloroform-d) δ 7.71 (dd, J = 18.1, 8.5 Hz, 2H), 7.50-7.45 (m, 2H), 7.37 (dd, J = 8.0, 1.7 Hz, 1H), 7.02 (d, J = 9.1 Hz, 1H), 6.19 (s, 1H), 5.18 (m, 1H), 5.02 (s, 2H), 4.45 (t, J = 7.0 Hz, 2H), 4.18 (s, 3H), 3.12 (t, J = 7.1 Hz, 2H), 1.45 (d, J = 6.6 Hz, 6H). | m/z: 512.1 [M + H]⁺ |
| 320 | 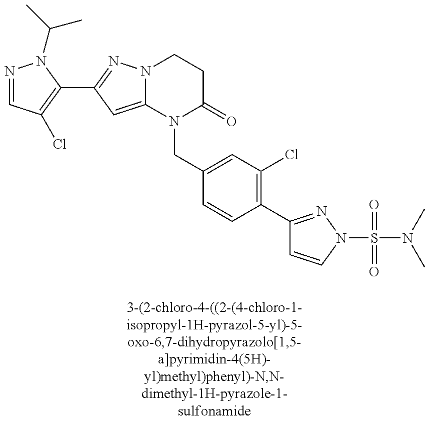 [SMILES: CC(C)n1ncc(Cl)c1-c1cc2n(n1)CCC(=O)N2Cc1ccc(-c2ccn(S(=O)(=O)N(C)C)n2)c(Cl)c1] | ¹H NMR (400 MHz, Chloroform-d) δ 8.00 (d, J = 2.6 Hz, 1H), 7.78 (d, J = 8.0 Hz, 1H), 7.48 (s, 1H), 7.44 (s, 1H), 7.32-7.28 (m, 1H), 6.86 (d, J = 2.6 Hz, 1H), 6.16 (s, 1H), 5.17 (m, 1H), 4.99 (s, 2H), 4.43 (t, J = 7.1 Hz, 2H), 3.10 (t, J = 7.1 Hz, 2H), 2.97 (s, 6H), 1.45 (d, J = 6.6 Hz, 6H). | m/z: 577.1 [M + H]⁺ |
| 321 | 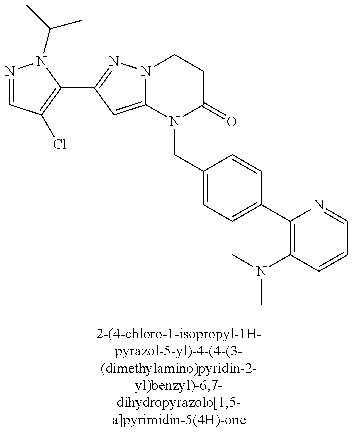 [SMILES: CC(C)n1ncc(Cl)c1-c1cc2n(n1)CCC(=O)N2Cc1ccc(-c2ncccc2N(C)C)cc1] | ¹H NMR (400 MHz, Chloroform-d) δ 8.25 (d, J = 3.6 Hz, 1H), 7.85 (d, J = 7.8 Hz, 2H), 7.47 (s, 1H), 7.36 (dd, J = 20.8, 7.9 Hz, 3H), 7.17 (d, J = 4.3 Hz, 1H), 6.16 (s, 1H), 5.15 (m, 1H), 5.04 (s, 2H), 4.43 (t, J = 7.0 Hz, 2H), 3.10 (t, J = 7.0 Hz, 2H), 2.57 (s, 6H), 1.44 (d, J = 6.6 Hz, 6H). | m/z: 490.1 [M + H]⁺ |
| 322 | 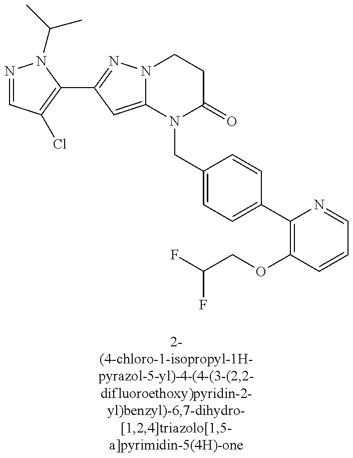 [SMILES: CC(C)n1ncc(Cl)c1-c1cc2n(n1)CCC(=O)N2Cc1ccc(-c2ncccc2OCC(F)F)cc1] | ¹H NMR (400 MHz, Chloroform-d) δ 8.38 (dd, J = 4.4, 1.5 Hz, 1H), 7.88-7.84 (m, 2H), 7.65-7.61 (m, 2H), 7.54 (s, 1H), 7.28 (dd, J = 8.4, 1.4 Hz, 1H), 7.24 (dd, J = 8.3, 4.4 Hz, 1H), 6.05 (tt, J = 54.9, 4.0 Hz, 1H), 5.27-5.0 (m, 3H), 4.40 (t, J = 7.3 Hz, 2H), 4.19 (td, J = 12.9, 4.0 Hz, 2H), 3.12 (t, J = 7.3 Hz, 2H), 1.50 (d, J = 6.6 Hz, 6H). | m/z: 528.1 [M + H]⁺ |

### Example 323 2-(4-chloro-1-((R)-1-fluoropropan-2-yl)-1H-pyrazol-5-yl)-N-(4-(1-ethyl-4-(trifluoromethyl)-1H-imidazol-2-yl)phenyl)-4,5,6,7-tetrahydropyrazolo[1,5-α]pyridin-4-amine and Example 324 2-(4-chloro-1-((S)-1-fluoropropan-2-yl)-1H-pyrazol-5-yl)-N-(4-(1-ethyl-4-(trifluoromethyl)-1H-imidazol-2-yl)phenyl)-4,5,6,7-tetrahydropyrazolo[1,5-α]pyridin-4-amine

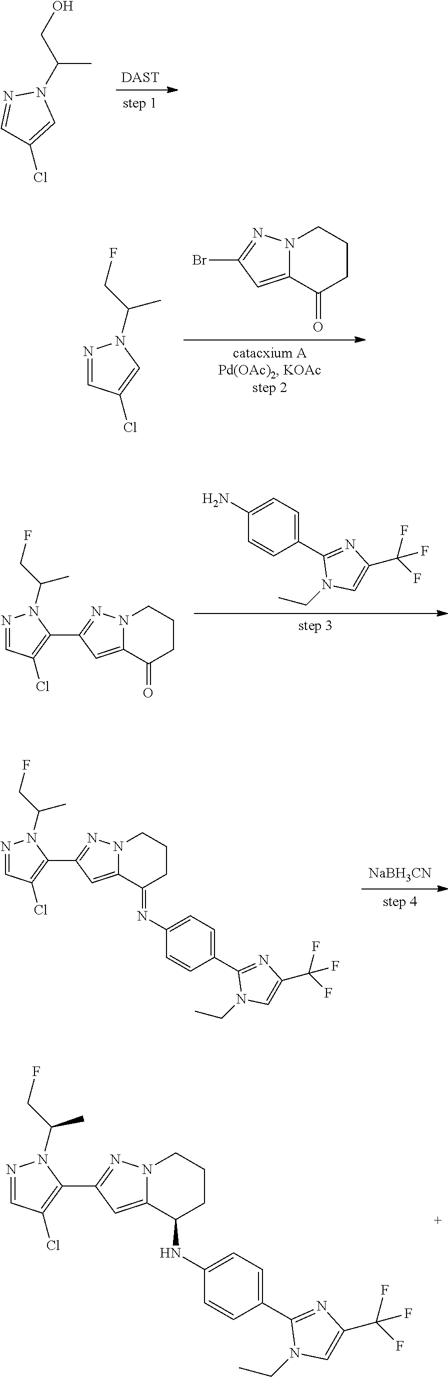

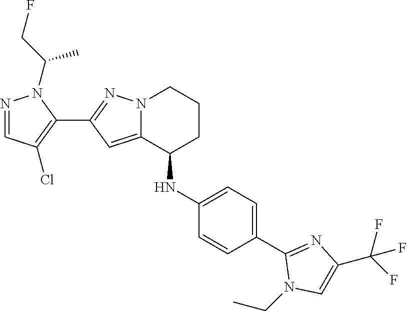 [SMILES: CCn1cc(C(F)(F)F)nc1-c1ccc(N[C@@H]2CCCn3nc(-c4c(Cl)cnn4[C@@H](C)CF)cc32)cc1]

### Step 1. 4-chloro-1-(1-fluoropropan-2-yl)-1H-pyrazole

To a solution of 2-(4-chloro-1H-pyrazol-1-yl)propan-1-ol (1.7 g, 10.62 mmol) in DCM (45 mL) was added diethylaminosulfur trifluoride (2.56 g, 15.94 mmol) at 0° C. The resulting mixture was stirred at room temperature for 4 days. The mixture was quenched with H₂O, concentrated and purified on a Biotage Isolera One (C₁₈ column, eluting with 10% to 90% MeCN in H₂O) to afford 4-chloro-1-(1-fluoropropan-2-yl)-1H-pyrazole (730 mg). MS (ESI) m/z 163.2 [M+H]⁺.

### Step 2. 2-(4-chloro-1-(1-fluoropropan-2-yl)-1H-pyrazol-5-yl)-6,7-dihydropyrazolo[1,5-α]pyridin-4(5H)-one

A mixture of 4-chloro-1-(1-fluoropropan-2-yl)-1H-pyrazole (510 mg, 3.15 mmol), 2-bromo-6,7-dihydropyrazolo[1,5-α]pyridin-4(5H)-one (564.1 mg, 2.62 mmol), Pd(OAc)₂ (58.4 mg, 0.26 mmol), catacxium A (186.4 mg, 0.52 mmol) and KOAc (770.3 mg, 7.86 mmol) in 2-Methyl-2-butanol (6 mL) was heated at 120° C. for two days under an atmosphere of N₂. The mixture was filtered through the celite, concentrated and purified by silica gel chromatography (eluting with 1/3 EtOAc/PE) to afford 2-(4-chloro-1-(1-fluoropropan-2-yl)-1H-pyrazol-5-yl)-6,7-dihydropyrazolo[1,5-α]pyridin-4(5H)-one (506 mg). MS (ESI) m/z 297.1 [M+H]⁺.

### Step 3. 2-(4-chloro-1-(1-fluoropropan-2-yl)-1H-pyrazol-5-yl)-N-(4-(1-ethyl-4-(trifluoromethyl)-1H-imidazol-2-yl) phenyl)-6,7-dihydropyrazolo[1,5-α]pyridin-4(5H)-imine

To a solution of 2-(4-chloro-1-(1-fluoropropan-2-yl)-1H-pyrazol-5-yl)-6,7-dihydropyrazolo[1,5-α]pyridin-4(5H)-one (176 mg, 0.59 mmol) in dry toluene (4 mL) was added 4-(1-ethyl-4-(trifluoromethyl)-1H-imidazol-2-yl)aniline (303.3 mg, 1.19 mmol) and titanium ethoxide (271.1 mg, 1.19 mmol) at room temperature. The resulting mixture was heated at 120° C. for 4 h in a microwave reactor. The mixture was concentrated and purified by Biotage (C₁₈ column, eluting with 10% to 90% MeCN in H₂O) to afford 2-(4-chloro-1-(1-fluoropropan-2-yl)-1H-pyrazol-5-yl)-N-(4-(1-ethyl-4-(trifluoromethyl)-1H-imidazol-2-yl)phenyl)-6,7-dihydropyrazolo[1,5-α]pyridin-4(5H)-imine (125 mg). MS (ESI) m/z 534.2 [M+H]⁺.

### Step 4. 2-(4-chloro-1-((R)-1-fluoropropan-2-yl)-1H-pyrazol-5-yl)-N-(4-(1-ethyl (trifluoromethyl)-1H-imidazol-2-yl)phenyl)-4,5,6,7-tetrahydropyrazolo[1,5-α]pyridin-4-amine and 2-(4-chloro-1-((S)-1-fluoropropan-2-yl)-1H-pyrazol-5-yl)-N-(4-(1-ethyl-4-(trifluoromethyl)-1H-imidazol-2-yl)phenyl)-4,5,6,7-tetrahydropyrazolo[1,5-α]pyridin-4-amine

To a solution of 2-(4-chloro-1-(1-fluoropropan-2-yl)-1H-pyrazol-5-yl)-N-(4-(1-ethyl-4-(trifluoromethyl)-1H-imidazol-2-yl)phenyl)-6,7-dihydropyrazolo[1,5-α]pyridin-4(5H)-imine (125 mg, 0.23 mmol) in MeOH (6 mL) was added NaBH₃CN (73.8 mg, 1.17 mmol) at room temperature. The resulting mixture was stirred at room temperature for 1 h. The mixture was quenched with H₂O, concentrated and purified by prep-HPLC (condition 3) to afford Example 323 2-(4-chloro-1-((R)-1-fluoropropan-2-yl)-1H-pyrazol-5-yl)-N-(4-(1-ethyl-4-(trifluoromethyl)-1H-imidazol-2-yl)phenyl)-4,5,6,7-tetrahydropyrazolo[1,5-α]pyridin-4-amine (19.6 mg) as the first eluting diastereomer (arbitrarily assigned stereochemistry). Further elution gave Example 324 2-(4-chloro-1-((S)-1-fluoropropan-2-yl)-1H-pyrazol-5-yl)-N-(4-(1-ethyl-4-(trifluoromethyl)-1 H-imidazol-2-yl)phenyl)-4,5,6,7-tetrahydropyrazolo[1,5-α]pyridin-4-amine (20.0 mg) as the second diastereomer with arbitrarily assigned stereochemistry.

Table 22. The compounds listed in Table 22 were synthesized in Example 323. The stereochemistries were arbitrarily assigned.

TABLE 22

| Example | | | |
|---|---|---|---|
| number | Structure | ¹H NMR (δ ppm) | MS (ESI) |
| 323 | 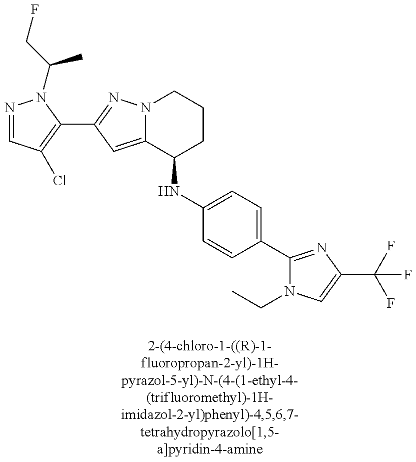 [SMILES: CCn1cc(C(F)(F)F)nc1-c1ccc(N[C@@H]2CCCn3nc(-c4c(Cl)cnn4[C@H](C)CF)cc32)cc1] | ¹H NMR (400 MHz, Methanol-d₄) δ 7.70 (s, 1H), 7.55 (s, 1H), 7.38 (d, J = 8.6 Hz, 2H), 6.89 (d, J = 8.7 Hz, 2H), 6.57 (s, 1H), 5.33 (d, J = 6.6 Hz, 1H), 4.95 (s, 1H), 4.80-4.62 (m, 1H), 4.47 (d, J = 4.5 Hz, 1H), 4.34-4.16 (m, 2H), 4.10 (d, J = 7.2 Hz, 2H), 2.37-2.27 (m, 2H), 2.18 (d, J = 9.8 Hz, 1H), 1.90 (d, J = 9.1 Hz, 1H), 1.45-1.41 (m, 3H), 1.38 (t, J = 7.3 Hz, 3H). | m/z: 536.1 [M + H]⁺ |
| 324 | 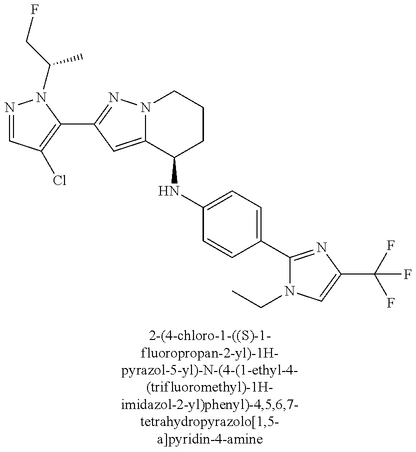 [SMILES: CCn1cc(C(F)(F)F)nc1-c1ccc(N[C@@H]2CCCn3nc(-c4c(Cl)cnn4[C@@H](C)CF)cc32)cc1] | ¹H NMR (400 MHz, Methanol-d₄) δ 7.70 (s, 1H), 7.55 (s, 1H), 7.38 (d, J = 8.6 Hz, 2H), 6.89 (d, J = 8.7 Hz, 2H), 6.57 (s, 1H), 5.33 (d, J = 6.6 Hz, 1H), 4.95 (s, 1H), 4.80-4.62 (m, 1H), 4.47 (d, J = 4.5 Hz, 1H), 4.34-4.16 (m, 2H), 4.10 (d, J = 7.2 Hz, 2H), 2.37-2.27 (m, 2H), 2.18 (d, J = 9.8 Hz, 1H), 1.90 (d, J = 9.1 Hz, 1H), 1.45-1.41 (m, 3H), 1.38 (t, J = 7.3 Hz, 3H). | m/z: 536.1 [M + H]⁺ |

### Example 325 (S)-5-(4-(1-(4-(1-ethyl-4-(trifluoromethyl)-1H-imidazol-2-yl)-3-fluorophenyl)ethyl)-5-oxo-4,5,6,7-tetrahydropyrazolo[1,5-α]pyrimidin-2-yl)-1-isopropyl-1H-pyrazole-4-carbonitrile and Example 326 (R)-5-(4-(1-(4-(1-ethyl-4-(trifluoromethyl)-1H-imidazol-2-yl)-3-fluorophenyl)ethyl)-5-oxo-4,5,6,7-tetrahydropyrazolo[1,5-α]pyrimidin-2-yl)-1-isopropyl-1H-pyrazole-4-carbonitrile

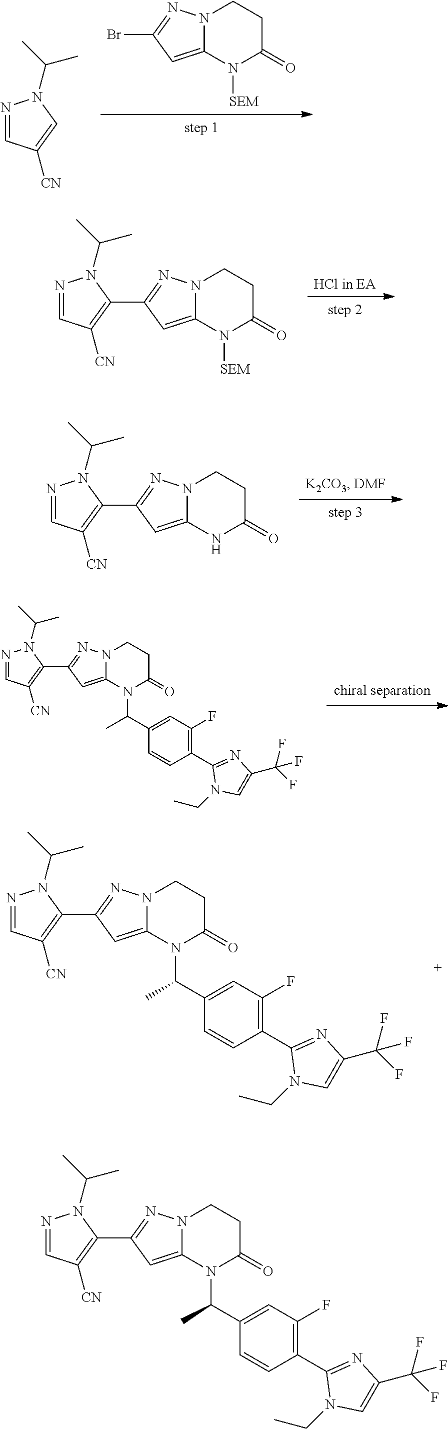

### Step 1. 1-isopropyl-5-(5-oxo-4-((2-(trimethylsilyl)ethoxy)methyl)-4,5,6,7-tetrahydropyrazolo[1,5-α]pyrimidin-2-yl)-1H-pyrazole-4-carbonitrile

1-isopropyl-5-(5-oxo-4-((2-(trimethylsilyl)ethoxy)methyl)-4,5,6,7-tetrahydropyrazolo[1,5-α]pyrimidin-2-yl)-1H-pyrazole-4-carbonitrile was prepared according to step 1 of Example 266. MS (ESI) m/z 401.2 [M+H]⁺.

### Step 2 and step 3. 5-(4-(1-(4-(1-ethyl-4-(trifluoromethyl)-1H-imidazol-2-yl)-3-fluorophenyl)ethyl)-5-oxo-4,5,6,7-tetrahydropyrazolo[1,5-α]pyrimidin-2-yl)-1-isopropyl-1H-pyrazole-4-carbonitrile

5-(4-(1-(4-(1-ethyl-4-(trifluoromethyl)-1H-imidazol-2-yl)-3-fluorophenyl)ethyl)-5-oxo-4,5,6,7-tetrahydropyrazolo[1,5-α]pyrimidin-2-yl)-1-isopropyl-1H-pyrazole-4-carbonitrile was synthesized following step 4 and step 5 of Example 154. The obtained racemate was purified by chiral chromatography (chiral preparative HPLC condition 2) to afford Example 325 (S)-5-(4-(1-(4-(1-ethyl-4-(trifluoromethyl)-1H-imidazol-2-yl)-3-fluorophenyl)ethyl)-5-oxo-4,5,6,7-tetrahydropyrazolo[1,5-α]pyrimidin-2-yl)-1-isopropyl-1H-pyrazole-4-carbonitrile as the first eluting compound. Further elution gave Example 326 (R)-5-(4-(1-(4-(1-ethyl-4-(trifluoromethyl)-1H-imidazol-2-yl)-3-fluorophenyl)ethyl)-5-oxo-4,5,6,7-tetrahydropyrazolo[1,5-α]pyrimidin-2-yl)-1-isopropyl-1H-pyrazole-4-carbonitrile. The absolute stereochemistries were assigned by comparing retention time on chiral HPLC with similar chiral compounds that were obtained via enantioselective synthesis.

Table 23. The compounds listed in Table 23 were synthesized in Example 325.

TABLE 23

| Example | | | |
|---|---|---|---|
| number | Structure | ¹H NMR (δ ppm) | MS (ESI) |
| 325 | 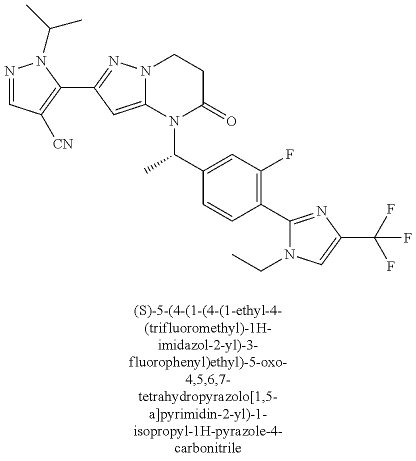 [SMILES: CCn1cc(C(F)(F)F)nc1-c1ccc([C@H](C)N2C(=O)CCn3nc(-c4c(C#N)cnn4C(C)C)cc32)cc1F] | ¹H NMR (400 MHz, DMSO- d₆) δ 8.08 (s, 2H), 7.54 (t, J = 7.7 Hz, 1H), 7.44 (d, J = 11.2 Hz, 1H), 7.36 (d, J = 7.9 Hz, 1H), 6.15 (q, J = 6.7 Hz, 1H), 5.92 (s, 1H), 5.17 (m, 1H), 4.49 (t, J = 6.7 Hz, 2H), 3.88 (q, J = 7.2 Hz, 2H), 3.23 (dd, J = 16.4, 8.2 Hz, 1H), 3.08- 2.98 (m, 1H), 1.76 (d, J = 7.0 Hz, 3H), 1.37 (t, J = 7.0 Hz, 6H), 1.25 (d, J = 7.2 Hz, 3H). | m/z: 555.1 [M + H]⁺ |
| 326 | 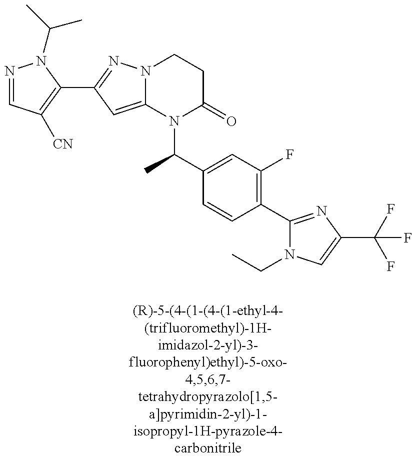 [SMILES: CCn1cc(C(F)(F)F)nc1-c1ccc([C@@H](C)N2C(=O)CCn3nc(-c4c(C#N)cnn4C(C)C)cc32)cc1F] | ¹H NMR (400 MHz, DMSO- d₆) δ 8.08 (s, 2H), 7.54 (t, J = 7.7 Hz, 1H), 7.44 (d, J = 11.2 Hz, 1H), 7.36 (d, J = 7.9 Hz, 1H), 6.15 (q, J = 6.7 Hz, 1H), 5.92 (s, 1H), 5.17 (m, 1H), 4.49 (t, J = 6.7 Hz, 2H), 3.88 (q, J = 7.2 Hz, 2H), 3.23 (dd, J = 16.4, 8.2 Hz, 1H), 3.08- 2.98 (m, 1H), 1.76 (d, J = 7.0 Hz, 3H), 1.37 (t, J = 7.0 Hz, 6H), 1.25 (d, J = 7.2 Hz, 3H). | m/z: 555.1 [M + H]⁺ |

### Example 327. 2-(4-cyclopropyl-6-methoxypyrimidin-5-yl)-4-(4-(1-ethyl-4-(trifluoromethyl)-1H-imidazol-2-yl)-3-fluorobenzyl)-6,6-dimethyl-4,5,6,7-tetrahydropyrazolo[1,5-α]pyrimidine

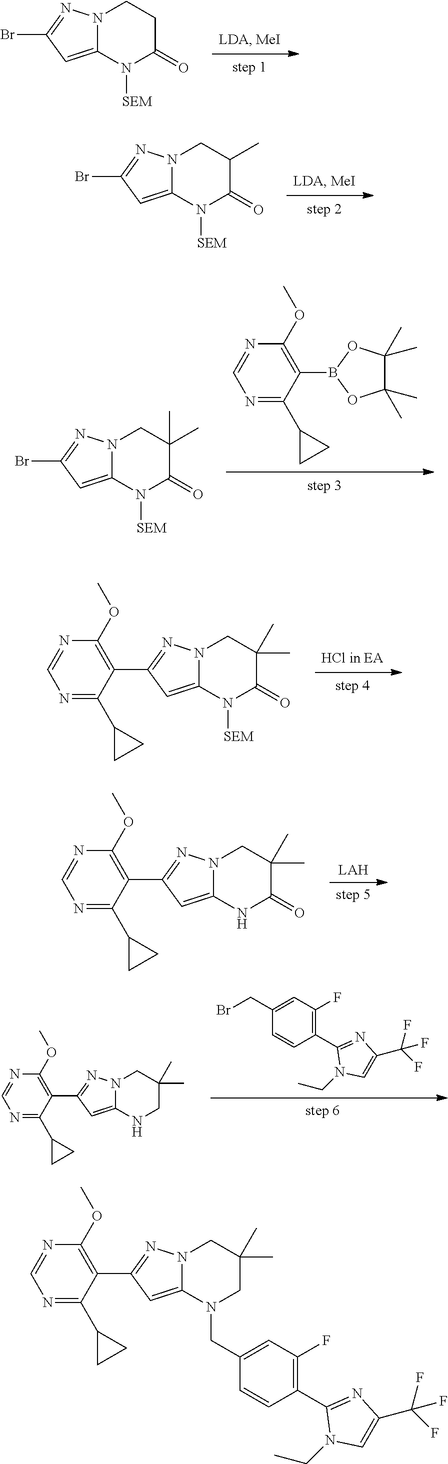

### Step 1. 2-bromo-6-methyl-4-((2-(trimethylsilyl)ethoxy)methyl)-6,7-dihydropyrazolo[1,5-α]pyrimidin-5(4H)-one

To a solution of 2-bromo-4-((2-(trimethylsilyl)ethoxy)methyl)-6,7-dihydropyrazolo[1,5-α]pyrimidin-5(4H)-one (723 mg, 2.1 mmol) in dry THF (10 mL) was added LDA (1 M in THF, 2.1 mL, 4.19 mmol) at −78° C. under an atmosphere of N₂. The mixture was stirred at −78° C. for 30 min then methyl iodide (595 mg, 4.19 mmol) was added. The resulting solution was warmed to room temperature and stirred for 1 h then quenched with saturated aqueous NH₄Cl (10 mL). The mixture was extracted with EtOAc (10 mL×3). The combined organic phases were washed with water and brine, dried over anhydrous Na₂SO₄, concentrated and purified by silica gel chromatography (eluting with 1/2 EtOAc/PE) to afford 2-bromo-6-methyl-4-((2-(trimethylsilyl)ethoxy)methyl)-6,7-dihydropyrazolo[1,5-α]pyrimidin-5(4H)-one (500 mg). MS (ESI) m/z 360.1 [M+H]⁺.

### Step 2. 2-bromo-6,6-dimethyl-4-((2-(trimethylsilyl)ethoxy)methyl)-6,7-dihydropyrazolo[1,5-α]pyrimidin-5(4H)-one

To a solution of 2-bromo-6-methyl-4-((2-(trimethylsilyl)ethoxy)methyl)-6,7-dihydropyrazolo[1,5-α]pyrimidin-5(4H)-one (500 mg, 1.39 mmol) in dry THF (6 mL) was added LDA (1 M in THF, 1.4 mL, 2.79 mmol) at −78° C. under an atmosphere of N₂. The mixture was stirred at −78° C. for 30 min then methyl iodide (400 mg, 2.79 mmol) was added. The resulting solution was warmed to room temperature and stirred for 1 h then quenched with saturated aqueous NH₄Cl (10 mL). The mixture was extracted with EtOAc (10 mL×3). The combined organic phases were washed with water and brine, dried over anhydrous Na₂SO₄, concentrated and purified by silica gel chromatography (eluting with 1/2 EtOAc/PE) to afford 2-bromo-6,6-dimethyl-4-((2-(trimethylsilyl)ethoxy) methyl)-6,7-dihydropyrazolo[1,5-α]pyrimidin-5(4H)-one (200 mg). MS (ESI) m/z 374.1 [M+H]⁺.

### Step 3. 2-(4-cyclopropyl-6-methoxypyrimidin-5-yl)-6,6-dimethyl-4-((2-(trimethylsilyl)ethoxy)methyl)-6,7-dihydropyrazolo[1,5-α]pyrimidin-5(4H)-one

To a solution of 2-bromo-6,6-dimethyl-4-((2-(trimethylsilyl)ethoxy)methyl)-6,7-dihydropyrazolo[1,5-α]pyrimidin-5(4H)-one (630 mg, 1.69 mmol) in a mixture of dioxane (15 mL) and H₂O (1.5 mL) was added 4-cyclopropyl-6-methoxy-5-(4,4,5,5-tetramethyl-1,3,2-dioxaborolan-2-yl) pyrimidine (933 mg, 3.38 mmol), Pd(dppf)Cl₂ (124 mg, 0.17 mmol) and K₃PO₄ (716 mg, 3.38 mmol) at room temperature. The resulting solution was stirred at 100° C. overnight under an atmosphere of N₂. The mixture was filtered through celite, concentrated and purified on a Biotage Isolera One (C₁₈ column, eluting with 10% to 90% MeCN in H₂O) to afford 2-(4-cyclopropyl-6-methoxypyrimidin-5-yl)-6,6-dimethyl-4-((2-(trimethylsilyl)ethoxy)methyl)-6,7-dihydropyrazolo[1,5-α]pyrimidin-5(4H)-one (720 mg). MS (ESI) m/z 444.3 [M+H]⁺.

### Step 4. 2-(4-cyclopropyl-6-methoxypyrimidin-5-yl)-6,6-dimethyl-6,7-dihydropyrazolo[1,5-α]pyrimidin-5(4H)-one

A solution of 2-(4-cyclopropyl-6-methoxypyrimidin-5-yl)-6,6-dimethyl (trimethylsilyl)ethoxy)methyl)-6,7-dihydropyrazolo[1,5-α]pyrimidin-5(4H)-one (700 mg, 1.58 mmol) in HCl EtOAc solution (4 M, 15 mL) was stirred at room temperature for 5 h. The solvent was removed and the residue diluted with EtOAc (50 mL), washed with 1M NaOH, dried over anhydrous Na₂SO₄, concentrated and purified by silica gel chromatography (eluting with 1/2 EtOAc/PE) to afford 2-(4-cyclopropyl-6-methoxypyrimidin-5-yl)-6,6-dimethyl-6,7-dihydropyrazolo[1,5-α]pyrimidin-5(4H)-one (180 mg). MS (ESI) m/z 314.2 [M+H]⁺.

### Step 5. and step 6. 2-(4-cyclopropyl-6-methoxypyrimidin-5-yl)-4-(4-(1-ethyl-4-(trifluoromethyl)-1H-imidazol-2-yl)-3-fluorobenzyl)-6,6-dimethyl-4,5,6,7-tetrahydropyrazolo[1,5-α]pyrimidine

Example 327 2-(4-cyclopropyl-6-methoxypyrimidin-5-yl)-4-(4-(1-ethyl-4-(trifluoromethyl)-1H-imidazol-2-yl)-3-fluorobenzyl)-6,6-dimethyl-4,5,6,7-tetrahydropyrazolo[1,5-α]pyrimidine was synthesized following step 2 and step 3 of Example 52. ¹H NMR (400 MHz, Methanol-d₄) δ 8.49 (s, 1H), 7.85 (d, J=1.3 Hz, 1H), 7.53 (t, J=7.6 Hz, 1H), 7.41-7.33 (m, 2H), 5.62 (s, 1H), 4.52 (s, 2H), 3.97 (q, J=7.3 Hz, 2H), 3.91 (s, 3H), 3.82 (s, 2H), 3.01 (s, 2H), 2.19 (dd, J=5.8, 3.5 Hz, 1H), 1.36 (d, J=7.3 Hz, 3H), 1.17 (s, 6H), 1.10 (dd, J=4.6, 2.8 Hz, 2H), 0.96-0.91 (m, 2H). MS (ESI) m/z 570.2 [M+H]⁺.

### Example 328. 2-(4-(difluoromethyl)-1-isopropyl-1H-pyrazol-5-yl)-4-(4-(1-ethyl-4-(trifluoromethyl)-1H-imidazol-2-yl)-3-fluorobenzyl)-6,7-dihydropyrazolo[1,5-α]pyrimidin-5(4H)-one

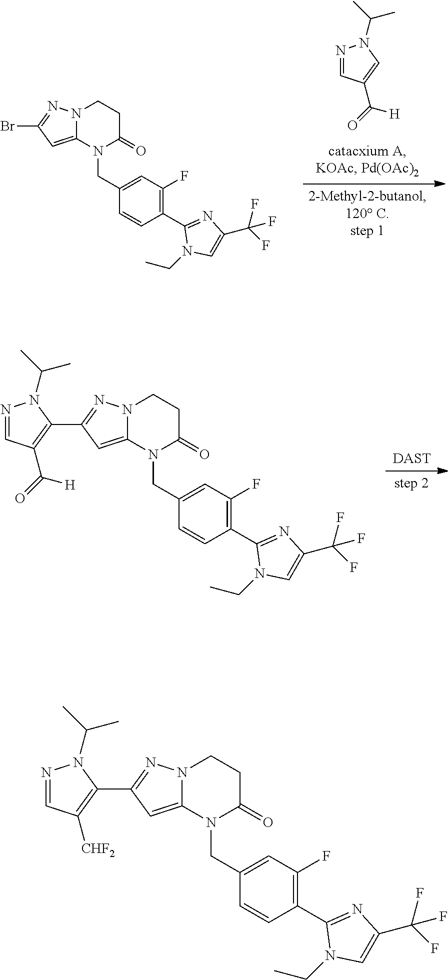

### Step 1 5-(4-(4-(1-ethyl-4-(trifluoromethyl)-1H-imidazol-2-yl)-3-fluorobenzyl)-5-oxo-4,5,6,7-tetrahydropyrazolo [1,5-α]pyrimidin-2-yl)-1-isopropyl-1H-pyrazole-4-carbaldehyde

A mixture of 2-bromo-4-(4-(1-ethyl-4-(trifluoromethyl)-1H-imidazol-2-yl)-3-fluorobenzyl)-6,7-dihydropyrazolo[1,5-α]pyrimidin-5(4H)-one (1.35 g, 2.78 mmol), 1-isopropyl-1H-pyrazole-4-carbaldehyde (274 mg, 1.98 mmol), catacxium A (144 mg, 0.4 mmol) and KOAc (980 mg, 10.0 mmol) in 2-Methyl-2-butanol (15 mL) was heated at 120° C. for two days in a sealed tube. The mixture was filtered through celite, concentrated and purified by silica gel chromatography (eluting with 1/1 EtOAc/PE) to afford 5-(4-(4-(1-ethyl-4-(trifluoromethyl)-1H-imidazol-2-yl)-3-fluorobenzyl)-5-oxo-4,5,6,7-tetrahydropyrazolo[1,5-α]pyrimidin-2-yl)-1-isopropyl-1H-pyrazole-4-carbaldehyde (175 mg). MS (ESI) m/z 544.2 [M+H]⁺.

### Step 2 2-(4-(difluoromethyl)-1-isopropyl-1H-pyrazol-5-yl)-4-(4-(1-ethyl-4-(trifluoromethyl)-1H-imidazol-2-yl)-3-fluorobenzyl)-6,7-dihydropyrazolo[1,5-α]pyrimidin-5(4H)-one

To a solution of 5-(4-(4-(1-ethyl-4-(trifluoromethyl)-1H-imidazol-2-yl)-3-fluorobenzyl)-5-oxo-4,5,6,7-tetrahydro-pyrazolo[1,5-α]pyrimidin-2-yl)-1-isopropyl-1H-pyrazole-4-carbaldehyde (70 mg, 0.13 mmol) in DCM (5 mL) was added DAST (311 mg, 1.93 mmol) at 0° C. The resulting solution was stirred at room temperature for 4 days then quenched with saturated aqueous NaHCO₃ (10 mL) and extracted with DCM (50 mL×3). The combined organic phases were washed with brine, dried over anhydrous Na₂SO₄, concentrated and purified by silica gel chromatography (eluting with 2/1 EtOAc/PE) to afford Example 328 2-(4-(difluoromethyl)-1-isopropyl-1H-pyrazol-5-yl)-4-(4-(1-ethyl-4-(trifluoromethyl)-1H-imidazol-2-yl)-3-fluorobenzyl)-6,7-dihydropyrazolo[1,5-α]pyrimidin-5(4H)-one (18.0 mg). ¹H NMR (400 MHz, Methanol-d₄) δ 7.87-7.85 (m, 1H), 7.72 (s, 1H), 7.52 (t, J=7.7 Hz, 1H), 7.36 (dd, J=7.9, 6.2 Hz, 2H), 6.68 (t, J=55.5 Hz, 1H), 6.08 (s, 1H), 5.15 (s, 2H), 4.94 (dd, J=13.3, 6.7 Hz, 1H), 4.50 (t, J=7.1 Hz, 2H), 3.94 (q, J=7.3 Hz, 2H), 3.16 (t, J=7.1 Hz, 2H), 1.43 (d, J=6.6 Hz, 6H), 1.34 (d, J=7.3 Hz, 3H). MS (ESI) m/z 566.2 [M+H]⁺.

### Example 329. 2-(4-(difluoromethoxy)-1-isopropyl-1H-pyrazol-5-yl)-4-(4-(1-ethyl-4-(trifluoromethyl)-1H-imidazol-2-yl)-3-fluorobenzyl)-6,7-dihydropyrazolo[1,5-α]pyrimidin-5(4H)-one

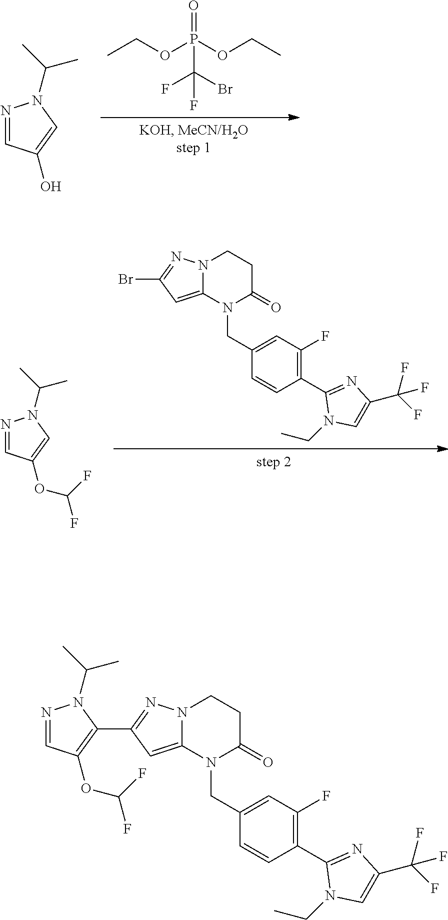

### Step 1. 4-(difluoromethoxy)-1-isopropyl-1H-pyrazole

To a solution of 1-isopropyl-1H-pyrazol-4-ol (3.2 g, 25.4 mmol) in MeCN (15 mL) and H₂O (15 mL) was added diethyl(bromodifluoromethyl)phosphonate (8.9 mL, 50.7 mmol) and KOH (28.5 g, 508 mmol) at −10° C. The resulting solution was stirred at room temperature overnight. The mixture was extracted with EtOAc (50 mL×3) and the combined organic phases washed with brine, concentrated and purified on a Biotage Isolera One (C₁₈ column, eluting with 10% to 90% MeCN in H₂O) to afford 4-(difluoromethoxy)-1-isopropyl-1H-pyrazole (704.4 mg). MS (ESI) m/z 177.1 [M+H]⁺.

### Step 2. 2-(4-(difluoromethoxy)-1-isopropyl-1H-pyrazol-5-yl)-4-(4-(1-ethyl-4-(trifluoromethyl)-1H-imidazol-2-yl)-3-fluorobenzyl)-6,7-dihydropyrazolo[1,5-α]pyrimidin-5(4H)-one

Example 329 2-(4-(difluoromethoxy)-1-isopropyl-1H-pyrazol-5-yl)-4-(4-(1-ethyl-4-(trifluoromethyl)-1H-imidazol-2-yl)-3-fluorobenzyl)-6,7-dihydropyrazolo[1,5-α]pyrimidin-5(4H)-one was synthesized according to step 1 of Example 328. ¹H NMR (400 MHz, Chloroform-d) δ 7.55 (s, 1H), 7.42 (d, J=13.8 Hz, 2H), 7.23 (s, 1H), 7.14 (d, J=10.1 Hz, 1H), 6.33 (t, J=74.3 Hz, 1H), 6.04 (s, 1H), 5.30 (d, J=6.1 Hz, 1H), 5.02 (s, 2H), 4.42 (d, J=6.7 Hz, 2H), 3.90 (d, J=7.2 Hz, 2H), 3.11 (s, 2H), 1.47 (d, J=6.3 Hz, 6H), 1.38 (t, J=7.1 Hz, 3H). MS (ESI) m/z 582.1 [M+H]⁺.

### Example 330. 2-(4-chloro-1-isopropyl-1H-pyrazol-5-yl)-N-(4-(1-ethyl (trifluoromethyl)-1H-imidazol-2-yl)benzyl)-4,5,6,7-tetrahydropyrazolo[1,5-α]pyridin amine

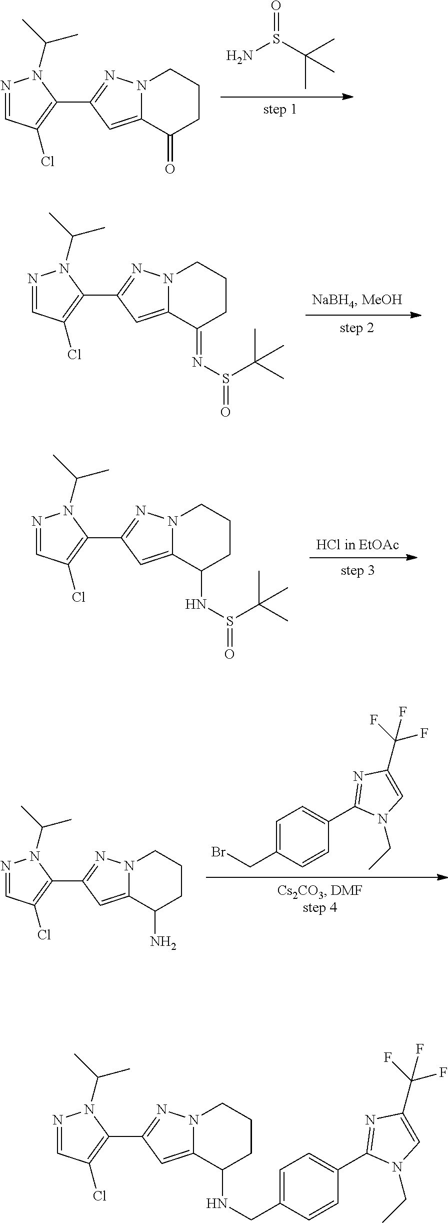

### Step 1: N-(2-(4-chloro-1-isopropyl-1H-pyrazol-5-yl)-6,7-dihydropyrazolo[1,5-α]pyridin-4(5H)-ylidene)-2-methylpropane-2-sulfinamide

To a solution of 2-(4-chloro-1-isopropyl-1H-pyrazol-5-yl)-6,7-dihydropyrazolo[1,5-α]pyridin-4(5H)-one (200 mg, 0.72 mmol) in toluene (5 mL) was added 2-methylpropane-2-sulfinamide (260 mg, 2.15 mmol). The resulting solution was stirred at 120° C. for 3 h in a sealed tube. The solvent was removed and the crude was purified on a Biotage Isolera One (C₁₈ column, eluting with 10% to 90% MeCN/H₂O) to afford ((E)-N-(2-(4-chloro-1-isopropyl-1H-pyrazol-5-yl)-6,7-dihydropyrazolo[1,5-α]pyridin-4(5H)-ylidene)-2-methylpropane-2-sulfinamide (100 mg). MS (ESI) m/z 382.1 [M+H]⁺.

### Step 2: N-(2-(4-chloro-1-isopropyl-1H-pyrazol-5-yl)-4,5,6,7-tetrahydropyrazolo[1,5-α]pyridin-4-yl)-2-methylpropane-2-sulfinamide

To a solution of N-(2-(4-chloro-1-isopropyl-1H-pyrazol-5-yl)-6,7-dihydropyrazolo[1,5-α]pyridin-4(5H)-ylidene)-2-methylpropane-2-sulfinamide (100 mg, 0.26 mmol) in MeOH (5 mL) was added NaBH4 (30 mg, 0.78 mmol) at room temperature. The resulting solution was stirred at room temperature for 1 h. Solvent was removed and the crude was purified on a Biotage Isolera One (C₁₈ column, eluting with 10% to 90% MeCN/H₂O) to afford N-(2-(4-chloro-1-isopropyl-1H-pyrazol-5-yl)-4,5,6,7-tetrahydropyrazolo[1,5-α]pyridin-4-yl)-2-methylpropane-2-sulfinamide (100 mg). MS (ESI) m/z 384.2 [M+H]⁺.

### Step 3: 2-(4-chloro-1-isopropyl-1H-pyrazol-5-yl)-4,5,6,7-tetrahydropyrazolo[1,5-α]pyridin-4-amine

A mixture of N-(2-(4-chloro-1-isopropyl-1H-pyrazol-5-yl)-4,5,6,7-tetrahydropyrazolo[1,5-α]pyridin-4-yl)-2-methylpropane-2-sulfinamide (110 mg, 0.29 mmol) in HCl EtOAc solution (4 M, 5 mL) was stirred at room temperature for 1 h. The reaction solution was concentrated under vacuum to give crude 2-(4-chloro-1-isopropyl-1H-pyrazol-5-yl)-4,5,6,7-tetrahydropyrazolo[1,5-α]pyridin-4-amine (80 mg) which was used directly in next step without further purification. MS (ESI) m/z 280.1 [M+H]⁺.

### Step 4: 2-(4-chloro-1-isopropyl-1H-pyrazol-5-yl)-N-(4-(1-ethyl-4-(trifluoromethyl)-1H-imidazol-2-yl)benzyl)-4,5,6,7-tetrahydropyrazolo[1,5-α]pyridin-4-amine

To a solution of 2-(4-chloro-1-isopropyl-1H-pyrazol-5-yl)-4,5,6,7-tetrahydropyrazolo[1,5-α]pyridin-4-amine (51.6 mg, 0.18 mmol), 2-(4-(bromomethyl)phenyl)-1-ethyl-4-(trifluoromethyl)-1H-imidazole (61.4 mg, 0.18 mmol) in DMF (5 mL) was added Cs₂CO₃ (117.4 mg, 0.36 mmol) at room temperature. The resulting solution was stirred at room temperature for 2 h then quenched with water (50 mL) and extracted with EtOAc (50 mL×3). The combined organic layers were washed with brine, dried over Na₂SO₄, concentrated under vacuum and purified by prep-TLC to give Example 330 2-(4-chloro-1-isopropyl-1H-pyrazol-5-yl)-N-(4-(1-ethyl-4-(trifluoromethyl)-1H-imidazol-2-yl) benzyl)-4,5,6,7-tetrahydropyrazolo[1,5-α]pyridin-4-amine (8.8 mg). ¹H NMR (400 MHz, Methanol-d₄) δ 7.78 (d, J=1.2 Hz, 1H), 7.59 (d, J=3.0 Hz, 4H), 7.53 (s, 1H), 6.72 (d, J=0.7 Hz, 1H), 5.12-5.03 (m, 1H), 4.22 (d, J=5.6 Hz, 2H), 4.15-4.06 (m, 3H), 4.01 (d, J=2.6 Hz, 2H), 2.39-2.30 (m, 1H), 2.29-2.20 (m, 1H), 2.02 (d, J=7.5 Hz, 1H), 1.91 (dd, J=10.3, 2.7 Hz, 1H), 1.44-1.35 (m, 9H). MS (ESI) m/z 532.2 [M+H]⁺.

### Example 331. 2-((2-(4-chloro-1-isopropyl-1H-pyrazol-5-yl)-4,5,6,7-tetrahydropyrazolo[1,5-α]pyridin-4-yl)(4-(1-ethyl-4-(trifluoromethyl)-1H-imidazol-2-yl)-3-fluorophenyl)amino)-ethan-1-ol

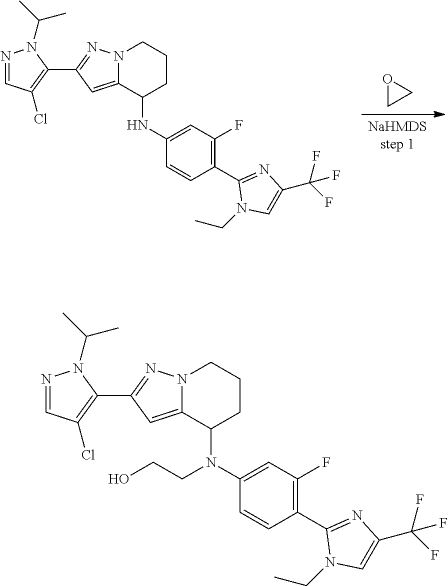

To a solution of 2-(4-chloro-1-isopropyl-1H-pyrazol-5-yl)-N-(4-(1-ethyl-4-(trifluoromethyl)-1H-imidazol-2-yl)-3-fluorophenyl)-4,5,6,7-tetrahydropyrazolo[1,5-α]pyridin-4-amine (prepared according to Example 237, 30 mg, 0.06 mmol) in dry THF (3 mL) was added ethylene oxide (1 mL) and NaHMDS (1 M in THF, 0.18 mL) at room temperature. The resulting mixture was stirred for 12 h in a sealed tube. The reaction was quenched with water and concentrated under vacuum. The residue was purified by prep-HPLC (condition 2) to give Example 331 2-((2-(4-chloro-1-isopropyl-1H-pyrazol-5-yl)-4,5,6,7-tetrahydropyrazolo[1,5-α]pyridin-4-yl)(4-(1-ethyl-4-(trifluoromethyl)-1H-imidazol-2-yl)-3-fluorophenyl)amino)ethan-1-ol (7.6 mg). ¹H NMR (400 MHz, Methanol-d₄) δ 7.80-7.75 (m, 1H), 7.50 (s, 1H), 7.32 (t, J=8.7 Hz, 1H), 6.88-6.72 (m, 2H), 6.36 (d, J=4.6 Hz, 1H), 5.32 (d, J=9.0 Hz, 1H), 5.13-5.03 (m, 1H), 4.59 (s, 1H), 4.40-4.30 (m, 1H), 4.26-4.17 (m, 1H), 3.96 (q, J=7.3 Hz, 2H), 3.75 (dd, J=9.9, 3.6 Hz, 2H), 3.49 (d, J=33.9 Hz, 1H), 3.42-3.35 (m, 1H), 2.37-2.13 (m, 4H), 1.40 (dd, J=6.6, 4.5 Hz, 6H), 1.35 (d, J=7.3 Hz, 3H). MS (ESI) m/z 580.2 [M+H]⁺.

### Example 332. 2-(4-cyclopropyl-6-methoxypyrimidin-5-yl)-4-(4-(1-ethyl-4-(trifluoromethyl)-1H-imidazol-2-yl)benzyl)pyrazolo[1,5-α]pyrimidin-5(4H)-one

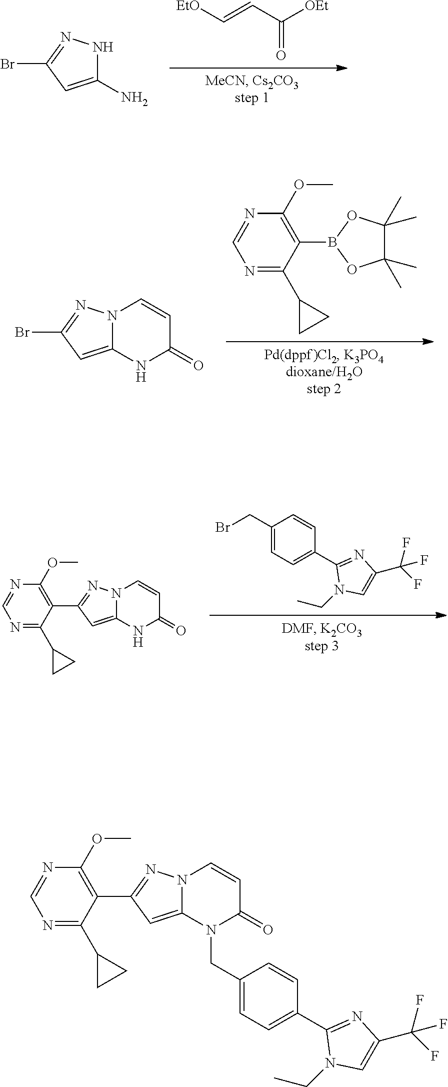

### Step 1. 2-bromopyrazolo[1,5-α]pyrimidin-5(4H)-one

A mixture of 3-bromo-1H-pyrazol-5-amine (3.22 g, 20.0 mmol), ethyl (E)-3-ethoxyacrylate (2.88 g, 20.0 mmol) and Cs₂CO₃ (652 mg, 2.0 mmol) in dioxane (5 mL) was heated at 110° C. for 2 h in microwave reactor. After cooling to ambient temperature, the mixture was filtered and the solid purified on a Biotage Isolera One (C₁₈ column, eluting with 10% to 95% MeCN/H₂O) to afford 2-bromopyrazolo[1,5-α]pyrimidin-5(4H)-one (1.23 g). MS (ESI) m/z 214.0, 216.0 [M+H]⁺.

### Step 2. 2-(4-cyclopropyl-6-methoxypyrimidin-5-yl)pyrazolo[1,5-α]pyrimidin-5(4H)-one

A mixture of 2-bromopyrazolo[1,5-α]pyrimidin-5(4H)-one (175 mg, 0.82 mmol), 4-cyclopropyl-6-methoxy-5-(4,4,5,5-tetramethyl-1,3,2-dioxaborolan-2-yl)pyrimidine (684 mg, 2.46 mmol), Pd(dppf)Cl₂ (58.5 mg, 0.08 mmol) and K₃PO₄ (356 mg, 1.64 mmol) in dioxane (5 mL) and H₂O (1 mL) was heated at reflux for 4 h under N₂. After cooling to ambient temperature, the mixture was filtered through celite and the filtrate concentrated under vacuum. The crude product was purified by silica gel chromatography (eluting with 1/1 EtOAc/PE) to afford 2-(4-cyclopropyl-6-methoxypyrimidin-5-yl)pyrazolo[1,5-α]pyrimidin-5(4H)-one (39.7 mg). MS (ESI) m/z 284.1 [M+H]⁺.

### Step 3. 2-(4-cyclopropyl-6-methoxypyrimidin-5-yl)-4-(4-(1-ethyl-4-(trifluoromethyl)-1H-imidazol-2-yl)benzyl) pyrazolo[1,5-α]pyrimidin-5(4H)-one

A mixture of 2-(4-cyclopropyl-6-methoxypyrimidin-5-yl)pyrazolo[1,5-α]pyrimidin-5(4H)-one (39.7 mg, 0.14 mmol), 2-(4-(bromomethyl)phenyl)-1-ethyl-4-(trifluoromethyl)-1H-imidazole (46.5 mg, 0.14 mmol) and K₂CO₃ (38.6 mg, 0.28 mmol) in DMF (3mL) was stirred at room temperature for 3 h. The mixture was filtered through celite and the filtrate concentrated under vacuum. The residue was purified on a Biotage Isolera One (C₁₈ column, eluting with 10% to 95% MeCN/H₂O, containing 0.1% formic acid) to afford Example 332 2-(4-cyclopropyl-6-methoxypyrimidin-5-yl)-4-(4-(1-ethyl-4-(trifluoromethyl)-1H-imidazol-2-yl)benzyl)pyrazolo[1,5-α]pyrimidin-5(4H)-one (10.9 mg). ¹H NMR (400 MHz, Chloroform-d) δ 8.59 (s, 1H), 8.17 (dd, J=7.8, 0.7 Hz, 1H), 7.58-7.54 (m, 2H), 7.50-7.46 (m, 2H), 7.36 (q, J=1.2 Hz, 1H), 6.20 (d, J=7.9 Hz, 1H), 6.00 (d, J=0.7 Hz, 1H), 5.28 (s, 2H), 4.04 (q, J=7.3 Hz, 2H), 3.92 (s, 3H), 2.14 (td, J=8.2, 4.1 Hz, 1H), 1.42 (t, J=7.3 Hz, 3H), 1.22-1.17 (m, 2H), 0.96-0.90 (m, 2H). MS (ESI) m/z 536.1 [M+H]⁺.

### Example 333. (R)-2-(4-cyclopropyl-6-methoxypyrimidin-5-yl)-4-(1-(4-(1-ethyl-4-(trifluoromethyl)-1H-imidazol-2-yl)-3-fluorophenyl)ethyl)pyrazolo[1,5-α]pyrimidin-5(4H)-one and Example 334 (S)-2-(4-cyclopropyl-6-methoxypyrimidin-5-yl)-4-(1-(4-(1-ethyl-4-(trifluoromethyl)-1H-imidazol-2-yl)-3-fluorophenyl)ethyl)pyrazolo[1,5-α]pyrimidin-5(4H)-one

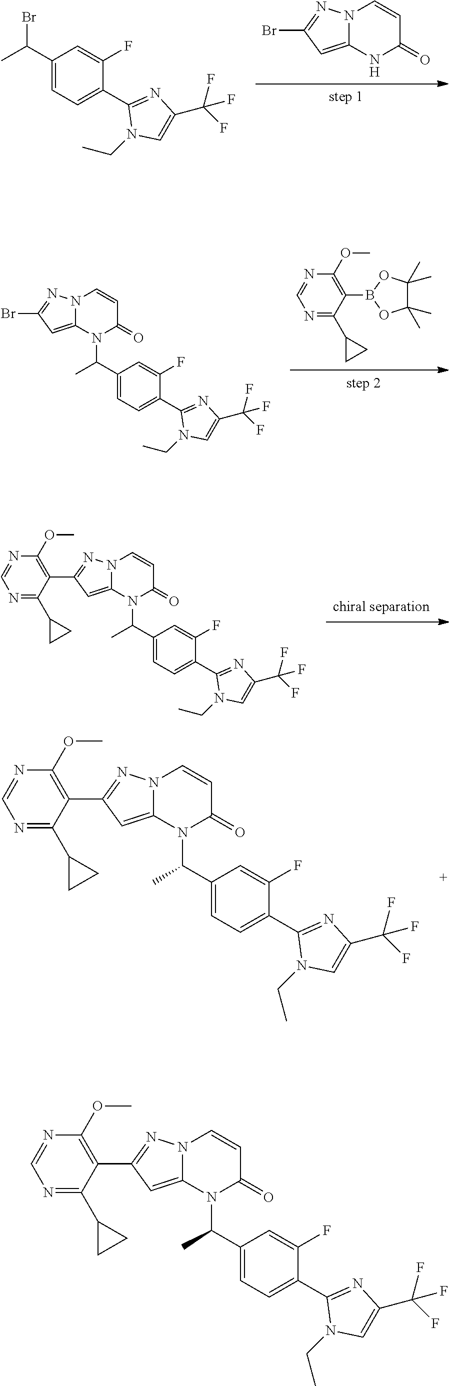

### Step 1. 2-bromo-4-(1-(4-(1-ethyl-4-(trifluoromethyl)-1H-imidazol-2-yl)-3-fluorophenyl)ethyl)pyrazolo[1,5-α]pyrimidin-5(4H)-one

To a solution of 2-(4-(1-bromoethyl)-2-fluorophenyl)-1-ethyl-4-(trifluoromethyl)-1H-imidazole (284 mg, 0.79 mmol) in MeCN (5 mL) was added 2-bromopyrazolo[1,5-α]pyrimidin-5(4H)-one (168.3, 0.79 mmol) and Cs₂CO₃ (514.8 mg, 1.58 mmol). The resulting mixture was heated at 80° C. for 2 h. After cooling to ambient temperature, the mixture was filtered through celite and the filtrate was concentrated under vacuum. The residue was purified by silica gel chromatography (eluting with 1/2 EtOAc/PE) to afford 2-bromo-4-(1-(4-(1-ethyl-4-(trifluoromethyl)-1H-imidazol-2-yl)-3-fluorophenyl)ethyl)pyrazolo[1,5-α]pyrimidin-5(4H)-one (150 mg). MS (ESI) m/z 498.0, 500.0 [M+H]⁺.

### Step 2. (R)-2-(4-cyclopropyl-6-methoxypyrimidin-5-yl)-4-(1-(4-(1-ethyl-4-(trifluoromethyl)-1H-imidazol-2-yl)-3-fluorophenyl)ethyl)pyrazolo[1,5-α]pyrimidin-5(4H)-one

To a solution of 2-bromo-4-(1-(4-(1-ethyl-4-(trifluoromethyl)-1H-imidazol-2-yl)-3-fluorophenyl)ethyl)pyrazolo[1,5-α]pyrimidin-5(4H)-one (150 mg, 0.30 mmol) in a mixture of dioxane (10 mL) and H₂O (1 mL) was added 4-cyclopropyl-6-methoxy-5-(4,4,5,5-tetramethyl-1,3,2-dioxaborolan-2-yl)pyrimidine (124.2 mg, 0.45 mmol), Pd(dppf)Cl₂ (36.6 mg, 0.05 mmol) and K₃PO₄ (130.2 mg, 0.60 mmol). The mixture was heated at 110° C. for 2 h under N₂ in a microwave reactor. After cooling to ambient temperature, the mixture was filtered through celite and the filtrate concentrated under vacuum. The residue was purified by Biotage (C₁₈ column, eluting with 10% to 95% MeCN/H₂O, containing 0.1% formic acid) to afford 2-(4-cyclopropyl-6-methoxypyrimidin-5-yl)-4-(1-(4-(1-ethyl-4-(trifluoromethyl)-1H-imidazol-2-yl)-3-fluorophenyl)ethyl)pyrazolo[1,5-α]pyrimidin-5(4H)-one (132 mg). The racemate was further separated by chiral prep-HPLC (chiral preparative HPLC condition 2) to give Example 334 (S)-2-(4-cyclopropyl-6-methoxypyrimidin-5-yl)-4-(1-(4-(1-ethyl-4-(trifluoromethyl)-1H-imidazol-2-yl)-3-fluorophenyl)-ethyl)pyrazolo[1,5-α]pyrimidin-5(4H)-one (53 mg) as the first eluting compound. Further elution provided Example 333 (R)-2-(4-cyclopropyl-6-methoxypyrimidin-5-yl)-4-(1-(4-(1-ethyl-4-(trifluoromethyl)-1H-imidazol-2-yl)-3-fluoro-phenyl)ethyl)pyrazolo[1,5-α]pyrimidin-5(4H)-one (55 mg). The absolute stereochemistries were assigned by comparing retention time on chiral HPLC with similar chiral compounds that were obtained via enantioselective synthetic methods.

Table 24. The compounds listed in Table 24 were synthesized according to Example 333 using the appropriate commercially available reagents and/or intermediates described above. Enantiomers were separated by chiral prep-HPLC and absolute stereochemistries assigned by comparing retention time on chiral HPLC with similar chiral compounds that were obtained via enantioselective synthesis.

TABLE 24

| Example | | | |
|---|---|---|---|
| number | Structure | ¹H NMR (δ ppm) | MS (ESI) |
| 333 | 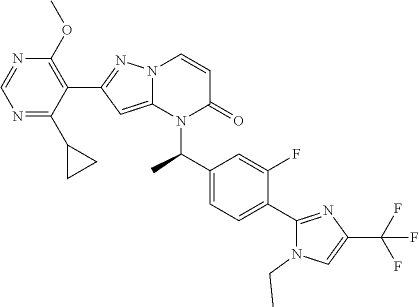 [SMILES: CCn1cc(C(F)(F)F)nc1-c1ccc([C@@H](C)n2c(=O)ccn3nc(-c4c(OC)ncnc4C4CC4)cc23)cc1F] | ¹H NMR (400 MHz, Methanol-d₄) δ 8.51 (s, 1H), 8.40 (d, J = 7.8 Hz, 1H), 7.86- 7.85 (m, 1H), 7.54 (t, J = 7.6 Hz, 1H), 7.40 (d, J = 9.5 Hz, 2H), 6.46 (d, J = 7.1 Hz, 1H), 6.21 (d, J = 7.8 Hz, 1H), 5.96 (s, 1H), 3.96 (d, J = 7.3 Hz, 2H), 3.87 (s, 3H), 2.17 (td, J = 8.0, 4.1 Hz, 1H), 1.95 (d, J = 7.1 Hz, 3H), 1.40-1.33 (m, 3H), 1.12-1.07 (m, 2H), 0.92- 0.89 (m, 2H). | m/z: 568.2 [M + H]⁺ |
| | (R)-2-(4-cyclopropyl-6-methoxypyrimidin-5-yl)-4-(1-(4-(1-ethyl-4- | | |
| | (trifluoromethyl)-1H-imidazol-2-yl)-3- | | |
| | fluorophenyl)ethyl)pyrazolo[1,5-a]pyrimidin-5(4H)-one | | |
| 334 | 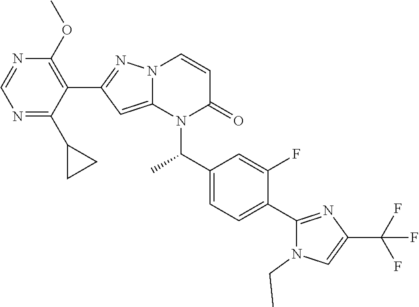 [SMILES: CCn1cc(C(F)(F)F)nc1-c1ccc([C@H](C)n2c(=O)ccn3nc(-c4c(OC)ncnc4C4CC4)cc23)cc1F] | ¹H NMR (400 MHz, Methanol-d₄) δ 8.51 (s, 1H), 8.40 (dd, J = 7.8, 0.7 Hz, 1H), 7.85 (q, J = 1.2 Hz, 1H), 7.54 (t, J = 7.6 Hz, 1H), 7.40 (d, J = 9.7 Hz, 2H), 6.46 (q, J = 7.0 Hz, 1H), 6.22 (d, J = 7.8 Hz, 1H), 5.96 (s, 1H), 3.95 (t, J = 7.2 Hz, 2H), 3.87 (s, 3H), 2.16 (m, 1H), 1.96 (d, J = 7.1 Hz, 3H), 1.35 (d, J = 7.4 Hz, 3H), 1.10 (dt, J = 4.8, 3.1 Hz, 2H), 0.90 (dd, J = 5.6, 2.8 Hz, 2H). | m/z: 568.2 [M + H]⁺ |
| | (S)-2-(4-cyclopropyl-6-methoxypyrimidin-5-yl)-4-(1-(4-(1-ethyl-4- | | |
| | (trifluoromethyl)-1H-imidazol-2-yl)-3- | | |
| | fluorophenyl)ethyl)pyrazolo[1,5-a]pyrimidin-5(4H)-one | | |
| 335 | 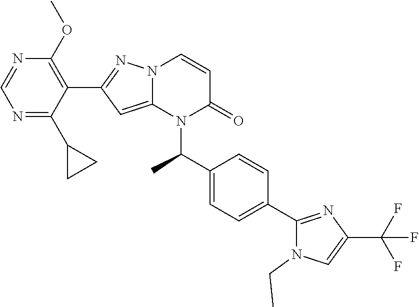 [SMILES: CCn1cc(C(F)(F)F)nc1-c1ccc([C@@H](C)n2c(=O)ccn3nc(-c4c(OC)ncnc4C4CC4)cc23)cc1] | ¹H NMR (400 MHz, Chloroform-d) δ 8.54 (d, J = 1.0 Hz, 1H), 8.15 (dt, J = 7.8, 0.9 Hz, 1H), 7.60-7.56 (m, 2H), 7.48-7.44 (m, 2H), 7.36 (q, J = 1.2 Hz, 1H), 6.61 (q, J = 7.1 Hz, 1H), 6.19 (dd, J = 7.8, 1.1 Hz, 1H), 5.59 (s, 1H), 4.05 (qd, J = 7.4, 1.1 Hz, 2H), 3.84 (d, J = 1.0 Hz, 3H), 2.06 (m, 1H), 1.91 (dd, J = 7.1, 1.2 Hz, 3H), 1.44-1.40 (m, 3H), 1.13 (pd, J = 5.2, 2.8 Hz, 2H), 0.91-0.83 (m, 2H). | m/z: 550.2 [M + H]⁺ |
| | (R)-2-(4-cyclopropyl-6-methoxypyrimidin-5-yl)-4-(1-(4-(1-ethyl-4- | | |
| | (trifluoromethyl)-1H-imidazol-2-yl)phenyl)ethyl)pyrazolo[1,5- | | |
| | a]pyrimidin-5(4H)-one | | |
| 336 | 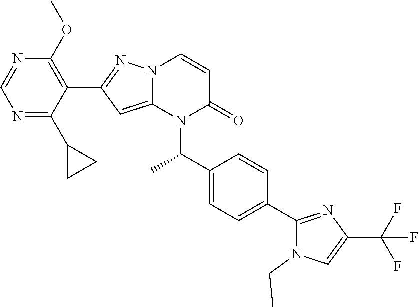 [SMILES: CCn1cc(C(F)(F)F)nc1-c1ccc([C@H](C)n2c(=O)ccn3nc(-c4c(OC)ncnc4C4CC4)cc23)cc1] | ¹H NMR (400 MHz, Chloroform-d) δ 8.54 (d, J = 1.0 Hz, 1H), 8.14 (dt, J = 7.8, 0.9 Hz, 1H), 7.60-7.56 (m, 2H), 7.48-7.44 (m, 2H), 7.35 (q, J = 1.2 Hz, 1H), 6.61 (q, J = 7.1 Hz, 1H), 6.19 (dd, J = 7.8, 1.1 Hz, 1H), 5.59 (s, 1H), 4.08-4.02 (m, 2H), 3.84 (d, J = 1.0 Hz, 3H), 2.09-2.04 (m, 1H), 1.91 (dd, J = 7.1, 1.2 Hz, 3H), 1.41 (td, J = 7.3, 1.1 Hz, 3H), 1.13 (qd, J = 5.0, 4.5, 1.9 Hz, 2H), 0.91-0.83 (m, 2H). | m/z: 550.2 [M + H]⁺ |
| | (S)-2-(4-cyclopropyl-6-methoxypyrimidin-5-yl)-4-(1-(4-(1-ethyl-4- | | |
| | (trifluoromethyl)-1H-imidazol-2-yl)phenyl)ethyl)pyrazolo[1,5- | | |
| | a]pyrimidin-5(4H)-one | | |

### Example 337. 2-(4-cyclopropyl-6-methoxypyrimidin-5-yl)-4-(4-(1-ethyl-4-(trifluoromethyl)-1H-imidazol-2-yl)benzyl)-[1,2,4]triazolo[1,5-α]pyrimidin-5(4H)-one

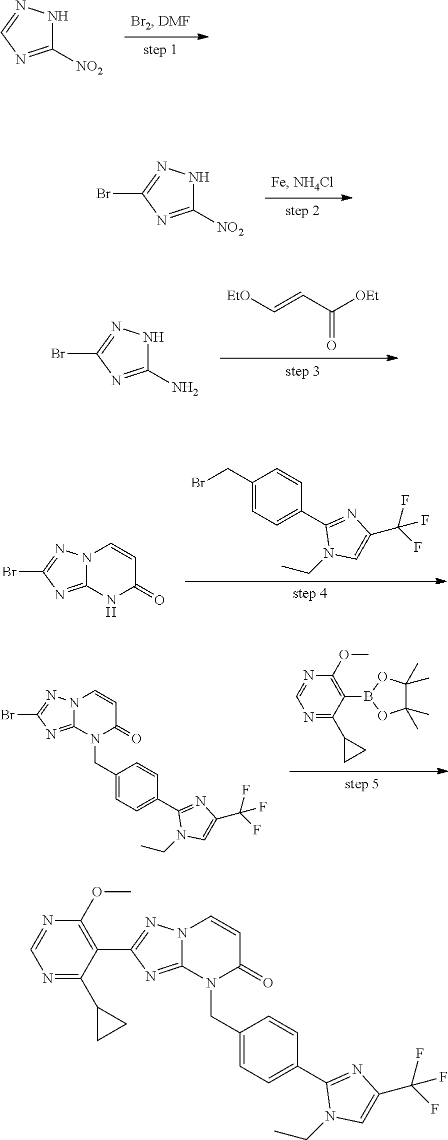

### Step 1. 3-bromo-5-nitro-1H-1,2,4-triazole

To a solution of 5-nitro-1H-1,2,4-triazole (20 g, 0.175 mol) in dry DMF (50 mL) was added Br₂ (18 mL) slowly over 30 min at room temperature. The resulting solution was heated to 40° C. overnight. After cooling to room temperature, the mixture was neutralized with saturated aqueous NaHCO₃ and extracted with EtOAc (50 mL×3). The combined organic phases were washed with water and brine, dried over anhydrous Na₂SO₄ and concentrated under vacuum to give crude 3-bromo-5-nitro-1H-1,2,4-triazole (12 g) which was used directly in next step without further purification. MS (ESI) m/z 192.9, 194.9 [M+H]⁺.

### Step 2. 3-bromo-1H-1,2,4-triazol-5-amine

A mixture of 3-bromo-5-nitro-1H-1,2,4-triazole (12 g, 62.5 mmol), iron power (21 g, 0.375 mol) and NH₄Cl (26.8 g, 0.5 mol) in a mixture of EtOH (40 mL) and H₂O (10 mL) was heated at 80° C. for 4 h. After cooling to room temperature, the reaction mixture was filtered through celite and the filtrate concentrated. The residue was dissolved in MeOH (50 mL) and filtered through a mixture of celite and silica gel. The filtrate was concentrated and dried under vacuum to give crude 3-bromo-1H-1,2,4-triazol-5-amine (5.5 g) which was used directly in next step without further purification. MS (ESI) m/z 163.0, 165.0 [M+H]⁺.

### Step 3. 2-bromo-[1,2,4]triazolo[1,5-α]pyrimidin-5(4H)-one

A mixture of 3-bromo-1H-1,2,4-triazol-5-amine (3 g, 18.5 mmol), ethyl (E)-3-ethoxyacrylate (2.7 g, 18.5 mmol) and K₂CO₃ in MeCN (20 mL) was heated at 95° C. in a sealed tube for 16 h. The reaction mixture was filtered through celite. The residue was washed with MeCN and dried under vacuum to give crude 2-bromo-[1,2,4]triazolo[1,5-α]pyrimidin-5(4H)-one (1.2 g) which was used directly in next step without further purification. MS (ESI) m/z 214.9, 216.9 [M+H]⁺.

### Step 4 and step 5. 2-(4-cyclopropyl-6-methoxypyrimidin-5-yl)-4-(4-(1-ethyl-4-(trifluoromethyl)-1H-imidazol-2-yl) benzyl)-[1,2,4]triazolo[1,5-α]pyrimidin-5(4H)-one

Example 337 2-(4-cyclopropyl-6-methoxypyrimidin-5-yl)-4-(4-(1-ethyl-4-(trifluoromethyl)-1H-imidazol-2-yl) benzyl)-[1,2,4]triazolo[1,5-α]pyrimidin-5(4H)-one was synthesized following step 2 and step 3 of Example 1.

Table 25. The compounds listed in Table 25 were synthesized according to Example 337 using the appropriate commercially available reagents and/or intermediates described above. Enantiomers, when generated, were separated by chiral prep-HPLC and absolute stereochemistries were assigned by comparing retention time on chiral HPLC with similar chiral compounds that were obtained via enantioselective synthesis.

TABLE 25

| Example | | | |
|---|---|---|---|
| number | Structure | ¹H NMR (δ ppm) | MS (ESI) |
| 337 | 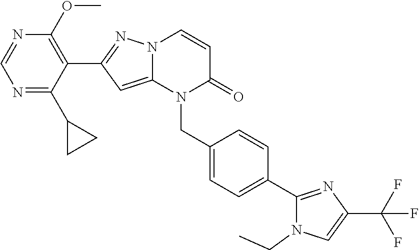 [SMILES: CCn1cc(C(F)(F)F)nc1-c1ccc(Cn2c(=O)ccn3nc(-c4c(OC)ncnc4C4CC4)cc23)cc1] | ¹H NMR (400 MHz, Chloroform-d) δ 8.67 (s, 1H), 8.19 (d, J = 7.8 Hz, 1H), 7.73 (d, J = 7.8 Hz, 2H), 7.53 (d, J = 7.8 Hz, 2H), 7.36 (s, 1H), 6.33 (d, J = 7.8 Hz, 1H), 5.47 (s, 2H), 4.04 (q, J = 7.3 Hz, 2H), 3.98 (s, 3H), 2.03 (dd, J = 8.8, 4.8 Hz, 1H), 1.42 (t, J = 7.1 Hz, 3H), 0.98 (dq, J = 6.6, 4.0 Hz, 2H), 0.91-0.85 (m, 2H). | m/z: 537.1 [M + H]⁺ |
| | 2-(4-cyclopropyl-6-methoxypyrimidin-5-yl)-4-(4-(1-ethyl-4- | | |
| | (trifluoromethyl)-1H-imidazol-2-yl)benzyl)- | | |
| | [1,2,4]triazolo[1,5-a]pyrimidin-5(4H)-one | | |
| 338 | 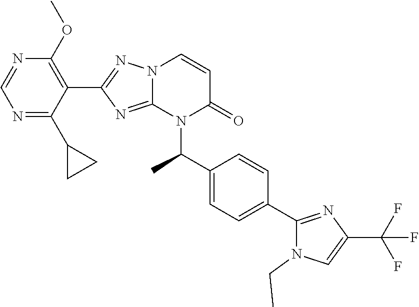 [SMILES: CCn1cc(C(F)(F)F)nc1-c1ccc([C@@H](C)n2c(=O)ccn3nc(-c4c(OC)ncnc4C4CC4)nc23)cc1] | ¹H NMR (400 MHz, Chloroform-d) δ 8.65 (s, 1H), 8.16 (d, J = 7.8 Hz, 1H), 7.69 (d, J = 7.9 Hz, 2H), 7.52 (d, J = 7.9 Hz, 2H), 7.35 (s, 1H), 6.51 (q, J = 7.3 Hz, 1H), 6.29 (d, J = 7.8 Hz, 1H), 4.05 (q, J = 7.3 Hz, 2H), 3.97 (s, 3H), 2.10 (d, J = 7.2 Hz, 3H), 2.01 (dq, J = 8.3, 4.3 Hz, 1H), 1.43 (t, J = 7.2 Hz, 3H), 1.25 (t, J = 10.4 Hz, 2H), 0.94 (dd, J = 8.1, 3.0 Hz, 2H). | m/z: 551.2 [M + H]⁺ |
| | (R)-2-(4-cyclopropyl-6-methoxypyrimidin-5-yl)-4-(1-(4-(1-ethyl-4- | | |
| | (trifluoromethyl)-1H-imidazol-2-yl)phenyl)ethyl)- | | |
| | [1,2,4]triazolo[1,5-a]pyrimidin-5(4H)-one | | |
| 339 | 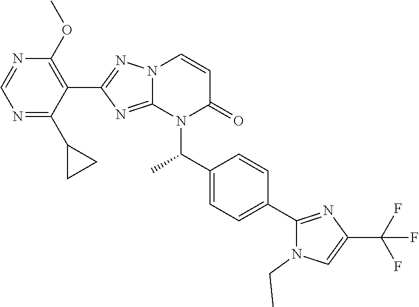 [SMILES: CCn1cc(C(F)(F)F)nc1-c1ccc([C@H](C)n2c(=O)ccn3nc(-c4c(OC)ncnc4C4CC4)nc23)cc1] | ¹H NMR (400 MHz, Chloroform-d) δ 8.65 (s, 1H), 8.16 (d, J = 7.8 Hz, 1H), 7.69 (d, J = 7.9 Hz, 2H), 7.52 (d, J = 7.9 Hz, 2H), 7.35 (s, 1H), 6.51 (q, J = 7.2 Hz, 1H), 6.29 (d, J = 7.8 Hz, 1H), 4.05 (q, J = 7.3 Hz, 2H), 3.97 (s, 3H), 2.10 (d, J = 7.2 Hz, 3H), 2.01 (dd, J = 8.2, 3.9 Hz, 1H), 1.43 (t, J = 7.2 Hz, 3H), 1.24 (d, J = 13.2 Hz, 2H), 0.94 (dd, J = 8.0, 3.0 Hz, 2H). | m/z: 551.2 [M + H]⁺ |
| | (S)-2-(4-cyclopropyl-6-methoxypyrimidin-5-yl)-4-(1-(4-(1-ethyl-4- | | |
| | (trifluoromethyl)-1H-imidazol-2-yl)phenyl)ethyl)- | | |
| | [1,2,4]triazolo[1,5-a]pyrimidin-5(4H)-one | | |
| 340 | 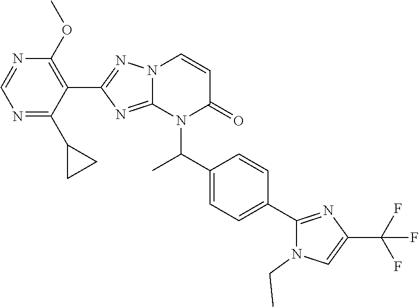 [SMILES: CCn1cc(C(F)(F)F)nc1-c1ccc(C(C)n2c(=O)ccn3nc(-c4c(OC)ncnc4C4CC4)nc23)cc1] | ¹H NMR (400 MHz, Chloroform-d) δ 8.64 (s, 1H), 8.16 (d, J = 7.8 Hz, 1H), 7.69 (d, J = 8.1 Hz, 2H), 7.52 (d, J = 8.3 Hz, 2H), 7.35 (s, 1H), 6.51 (q, J = 7.3, 6.9 Hz, 1H), 6.29 (d, J = 7.8 Hz, 1H), 4.05 (q, J = 7.3 Hz, 2H), 3.97 (s, 3H), 2.10 (d, J = 7.3 Hz, 3H), 2.01 (dt, J = 8.3, 3.9 Hz, 1H), 1.43 (t, J = 7.3 Hz, 3H), 1.25- 1.15 (m, 2H), 0.93 (dd, J = 8.0, 3.0 Hz, 2H). | m/z: 551.2 [M + H]⁺ |
| | 2-(4-cyclopropyl-6-methoxypyrimidin-5-yl)-4-(1-(4-(1-ethyl-4- | | |
| | (trifluoromethyl)-1H-imidazol-2-yl)phenyl)ethyl)- | | |
| | [1,2,4]triazolo[1,5-a]pyrimidin-5(4H)-one | | |
| 341 | 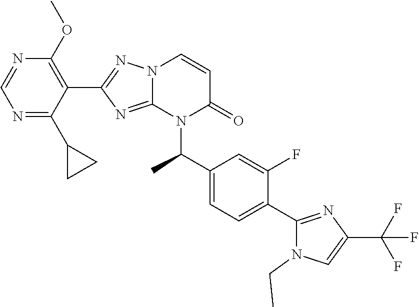 [SMILES: CCn1cc(C(F)(F)F)nc1-c1ccc([C@@H](C)n2c(=O)ccn3nc(-c4c(OC)ncnc4C4CC4)nc23)cc1F] | ¹H NMR (400 MHz, Chloroform-d) δ 8.66 (s, 1H), 8.16 (d, J = 7.8 Hz, 1H), 7.55- 7.45 (m, 3H), 7.38 (s, 1H), 6.49 (q, J = 7.2 Hz, 1H), 6.28 (d, J = 7.8 Hz, 1H), 3.98 (s, 3H), 3.91 (q, J = 7.3 Hz, 2H), 2.10 (d, J = 7.2 Hz, 3H), 2.05 (q, J = 4.3 Hz, 1H), 1.39 (t, J = 7.3 Hz, 3H), 1.24 (dd, J = 7.4, 4.0 Hz, 2H), 0.97 (d, J = 8.4 Hz, 2H). | m/z: 569.2 [M + H]⁺ |
| | (R)-2-(4-cyclopropyl-6-methoxypyrimidin-5-yl)-4-(1-(4-(1-ethyl-4- | | |
| | (trifluoromethyl)-1H-imidazol-2-yl)-3-fluorophenyl)ethyl)- | | |
| | [1,2,4]triazolo[1,5-a]pyrimidin-5(4H)-one | | |
| 342 | 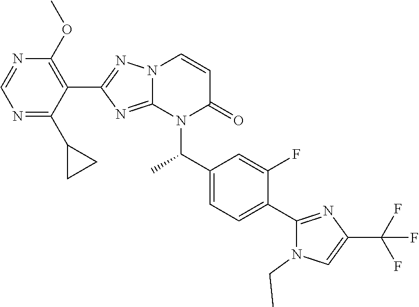 [SMILES: CCn1cc(C(F)(F)F)nc1-c1ccc([C@H](C)n2c(=O)ccn3nc(-c4c(OC)ncnc4C4CC4)nc23)cc1F] | ¹H NMR (400 MHz, Chloroform-d) δ 8.66 (s, 1H), 8.16 (dd, J = 7.8, 1.0 Hz, 1H), 7.55-7.45 (m, 3H), 7.40-7.37 (m, 1H), 6.49 (q, J = 7.2 Hz, 1H), 6.28 (dd, J = 7.8, 1.0 Hz, 1H), 3.98 (d, J = 0.9 Hz, 3H), 3.91 (q, J = 7.3 Hz, 2H), 2.10 (dd, J = 7.4, 1.0 Hz, 3H), 2.05 (dt, J = 8.0, 3.9 Hz, 1H), 1.39 (t, J = 7.3 Hz, 3H), 1.27-1.19 (m, 2H), 0.97 (d, J = 8.2 Hz, 2H). | m/z: 569.2 [M + H]⁺ |
| | (S)-2-(4-cyclopropyl-6-methoxypyrimidin-5-yl)-4-(1-(4-(1-ethyl-4- | | |
| | (trifluoromethyl)-1H-imidazol-2-yl)-3-fluorophenyl)ethyl)- | | |
| | [1,2,4]triazolo[1,5-a]pyrimidin-5(4H)-one | | |
| 343 | 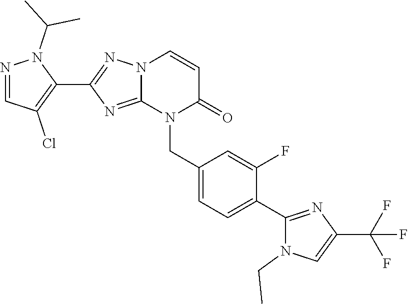 [SMILES: CCn1cc(C(F)(F)F)nc1-c1ccc(Cn2c(=O)ccn3nc(-c4c(Cl)cnn4C(C)C)nc23)cc1F] | ¹H NMR (400 MHz, Chloroform-d) δ 8.22 (d, J = 7.9 Hz, 1H), 7.58 (s, 1H), 7.55-7.52 (m, 2H), 7.49 (d, J = 10.5 Hz, 1H), 7.41-7.37 (m, 1H), 6.35 (d, J = 7.9 Hz, 1H), 5.44 (s, 2H), 5.32 (m, 1H), 3.88 (q, J = 7.3 Hz, 2H), 1.52 (d, J = 6.6 Hz, 6H), 1.36 (t, J = 7.3 Hz, 3H). | m/z: 549.1 [M + H]⁺ |
| | 2-(4-chloro-1-isopropyl-1H-pyrazol-5-yl)-4-(4-(1-ethyl-4- | | |
| | (trifluoromethyl)-1H-imidazol-2-yl)-3-fluorobenzyl)- | | |
| | [1,2,4]triazolo[1,5-a]pyrimidin-5(4H)-one | | |
| 344 | 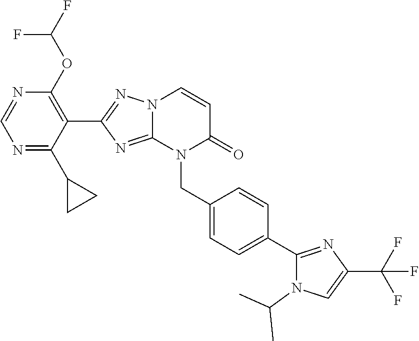 [SMILES: CC(C)n1cc(C(F)(F)F)nc1-c1ccc(Cn2c(=O)ccn3nc(-c4c(OC(F)F)ncnc4C4CC4)nc23)cc1] | ¹H NMR (400 MHz, Chloroform-d) δ 8.68 (s, 1H), 8.19 (s, 1H), 7.77 (s, 2H), 7.56 (t, J = 71.6 Hz, 1H), 7.50 (s, 2H), 7.40 (s, 1H), 6.35 (s, 1H), 5.46 (s, 2H), 4.52 (s, 1H), 2.25 (s, 1H), 1.43 (s, 6H), 1.32 (s, 2H), 1.08 (s, 2H). | m/z: 587.1 [M + H]⁺ |
| | 2-(4-cyclopropyl-6-(difluoromethoxy)pyrimidin-5-yl)-4-(4-(1- | | |
| | isopropyl-4-(trifluoromethyl)-1H-imidazol-2-yl)benzyl)- | | |
| | [1,2,4]triazolo[1,5-a]pyrimidin-5(4H)-one | | |
| 345 | 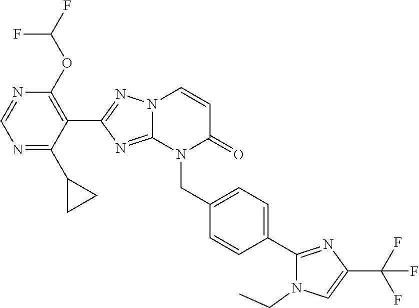 [SMILES: CCn1cc(C(F)(F)F)nc1-c1ccc(Cn2c(=O)ccn3nc(-c4c(OC(F)F)ncnc4C4CC4)nc23)cc1] | ¹H NMR (400 MHz, Chloroform-d) δ 8.69 (s, 1H), 8.19 (d, J = 7.8 Hz, 1H), 7.77 (d, J = 7.8 Hz, 2H), 7.57 (t, J = 71.6 Hz, 1H), 7.54 (d, J = 7.8 Hz, 2H), 7.35 (s, 1H), 6.35 (d, J = 7.8 Hz, 1H), 5.46 (s, 2H), 4.03 (q, J = 7.1 Hz, 2H), 2.24 (s, 1H), 1.42 (t, J = 7.2 Hz, 3H), 1.32 (s, 2H), 1.10-1.04 (m, 2H). | m/z: 573.1 [M + H]⁺ |
| | 2-(4-cyclopropyl-6-(difluoromethoxy)pyrimidin-5-yl)-4-(4-1-ethyl-4- | | |
| | (trifluoromethyl)-1H-imidazol-2-yl)benzyl)- | | |
| | [1,2,4]triazolo[1,5-a]pyrimidin-5(4H)-one | | |
| 346 | 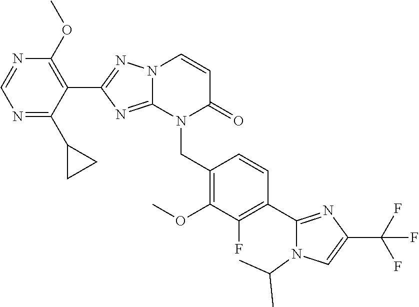 [SMILES: COc1ncnc(C2CC2)c1-c1nc2n(Cc3ccc(-c4nc(C(F)(F)F)cn4C(C)C)c(F)c3OC)c(=O)ccn2n1] | ¹H NMR (400 MHz, Chloroform-d) δ 8.66 (s, 1H), 8.25 (d, J = 7.8 Hz, 1H), 7.42 (s, 1H), 7.12 (d, J = 5.5 Hz, 2H), 6.34 (d, J = 7.8 Hz, 1H), 5.53 (s, 2H), 4.24 (m, 1H), 4.02 (d, J = 2.1 Hz, 3H), 3.96 (s, 3H), 2.00 (tt, J = 8.1, 4.6 Hz, 1H), 1.41 (d, J = 6.7 Hz, 6H), 1.25-1.21 (m, 2H), 0.97 (dq, J = 7.1, 3.9 Hz, 2H). | m/z: 599.3 [M + H]⁺ |
| | 2-(4-cyclopropyl-6-methoxypyrimidin-5-yl)-4-(3-fluoro-4-(1-isopropyl- | | |
| | 4-(trifluoromethyl)-1H-imidazol-2-yl)-2-methoxybenzyl)- | | |
| | [1,2,4]triazolo[1,5-a]pyrimidin-5(4H)-one | | |

### Example 347. 2-(4-cyclopropyl-6-methoxypyrimidin-5-yl)-4-(4-(1-ethyl-4-(trifluoromethyl)-1H-imidazol-2-yl)-3-fluorobenzyl)-6-methyl-6,7-dihydro-[1,2,4]triazolo[1,5-α]pyrimidin-5(4H)-one

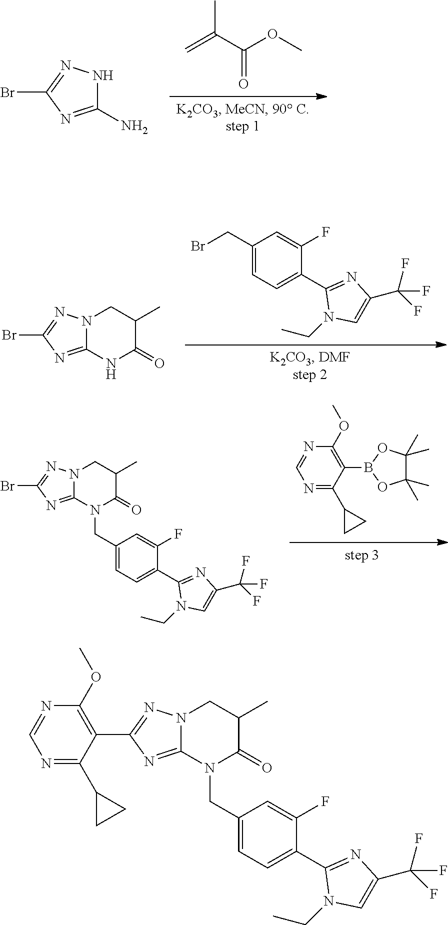

### Step 1. 2-bromo-6-methyl-6,7-dihydro-[1,2,4]triazolo[1,5-α]pyrimidin-5(4H)-one

A mixture of 3-bromo-1H-1,2,4-triazol-5-amine (300 mg, 1.8 mmol), methyl methacrylate (210 mg, 1.8 mmol) and K₂CO₃ (508 mg, 3.6 mmol) in MeCN (6 mL) was heated at 90° C. overnight in a sealed tube. The mixture was concentrated and purified by silica gel chromatography (eluting with 1/1 EtOAc/PE) to afford 2-bromo-6-methyl-6,7-dihydro-[1,2,4]triazolo[1,5-α]pyrimidin-5(4H)-one (157 mg). MS (ESI) m/z 231.0, 233.0 [M+H]⁺.

### Step 2. 2-bromo-4-(4-(1-ethyl-4-(trifluoromethyl)-1H-imidazol-2-yl)-3-fluorobenzyl)-6-methyl-6,7-dihydro-[1,2,4]triazolo[1,5-α]pyrimidin-5(4H)-one

A mixture of 2-bromo-6-methyl-6,7-dihydro-[1,2,4]triazolo[1,5-α]pyrimidin-5(4H)-one (100 mg, 0.43 mmol), 2-(4-(bromomethyl)-2-fluorophenyl)-1-ethyl-4-(trifluoromethyl)-1H-imidazole (182 mg, 0.52 mmol) and K₂CO₃ (119 mg, 0.86 mmol) in DMF (2 mL) was heated at 80° C. for 30 min. The resulting solution was diluted with water (20 mL) and extracted with EtOAc (20 mL×3). The combined organic layers were washed with brine, dried over Na₂SO₄, concentrated under vacuum and purified by silica gel chromatography (eluting with 1/2 EtOAc/PE) to afford 2-bromo-4-(4-(1-ethyl-4-(trifluoromethyl)-1H-imidazol-2-yl)-3-fluorobenzyl)-6-methyl-6,7-dihydro-[1,2,4]triazolo[1,5-α]pyrimidin-5(4H)-one (189 mg). MS (ESI) m/z 501.0, 503.0 [M+H]⁺.

### Step 3. 2-(4-cyclopropyl-6-methoxypyrimidin-5-yl)-4-(4-(1-ethyl-4-(trifluoromethyl)-1H-imidazol-2-yl)-3-fluorobenzyl)-6-methyl-6,7-dihydro-[1,2,4]triazolo[1,5-α]pyrimidin-5(4H)-one

A mixture of 4-cyclopropyl-6-methoxy-5-(4,4,5,5-tetramethyl-1,3,2-dioxaborolan-2-yl)pyrimidine (82 mg, 0.3 mmol), 2-bromo-4-(4-(1-ethyl-4-(trifluoromethyl)-1H-imidazol-2-yl)-3-fluorobenzyl)-6-methyl-6,7-dihydro-[1,2,4] triazolo[1,5-α]pyrimidin-5(4H)-one (100 mg, 0.2 mmol), Cs₂CO₃ (130 mg, 0.4 mmol), Pd₂(dba)₃ (18 mg, 0.02 mmol) and X-Phos (19 mg, 0.04 mmol) in toluene (2 mL) and H₂O (0.2 mL) was heated at 100° C. for 4 h under an atmosphere of N₂ in a sealed tube. The resulting mixture was filtered through celite, concentrated and purified by silica gel chromatography (eluting with 1/1 EtOAc/PE) to afford Example 347 2-(4-cyclopropyl-6-methoxypyrimidin-5-yl)-4-(4-(1-ethyl-4-(trifluoromethyl)-1H-imidazol-2-yl)-3-fluorobenzyl)-6-methyl-6,7-dihydro-[1,2,4]triazolo[1,5-α]pyrimidin-5(4H)-one (19.2 mg).

Table 26. The compounds listed in Table 26 were synthesized according to Example 347 using the appropriate commercially available reagents and/or intermediates described above. Enantiomers, when generated, were separated by chiral prep-HPLC and absolute stereochemistries stereochemistries were arbitrarily assigned.

TABLE 26

| Example | | | |
|---|---|---|---|
| number | Structure | ¹H NMR (δ ppm) | MS (ESI) |
| 347 | 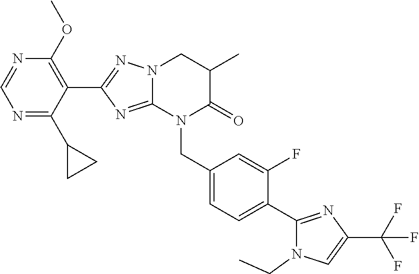 [SMILES: CCn1cc(C(F)(F)F)nc1-c1ccc(CN2C(=O)C(C)Cn3nc(-c4c(OC)ncnc4C4CC4)nc32)cc1F] | ¹H NMR (400 MHz, DMSO- d₆) δ 8.65 (s, 1H), 8.11 (d, J = 1.1 Hz, 1H), 7.52 (t, J = 7.8 Hz, 1H), 7.42 (d, J = 10.9 Hz, 1H), 7.35 (dd, J = 8.0, 1.4 Hz, 1H), 5.11 (q, J = 15.8 Hz, 2H), 4.56 (dd, J = 12.4, 7.6 Hz, 1H), 4.15 (t, J = 12.1 Hz, 1H), 3.86 (d, J = 2.8 Hz, 5H), 3.40 (dt, J = 11.7, 7.2 Hz, 1H), 2.04-1.97 (m, 1H), 1.26 (dt, J = 7.3, 3.8 Hz, 6H), 1.05- 1.00 (m, 2H), 0.93-0.86 (m, 2H). | m/z: 571.2 [M + H]⁺ |
| | 2-(4-cyclopropyl-6-methoxypyrimidin-5-yl)-4-(4-(1-ethyl-4- | | |
| | (trifluoromethyl)-1H-imidazol-2-yl)-3-fluorobenzyl)-6-methyl-6,7- | | |
| | dihydro-[1,2,4]triazolo[1,5-a]pyrimidin-5(4H)-one | | |
| 348 | 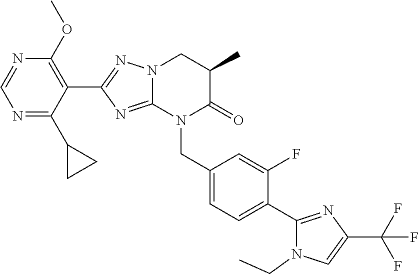 [SMILES: CCn1cc(C(F)(F)F)nc1-c1ccc(CN2C(=O)[C@H](C)Cn3nc(-c4c(OC)ncnc4C4CC4)nc32)cc1F] | ¹H NMR (400 MHz, Chloroform-d) δ 8.62 (s, 1H), 7.49 (t, J = 7.5 Hz, 1H), 7.45- 7.38 (m, 3H), 5.18 (d, J = 1.8 Hz, 2H), 4.51 (dd, J = 13.0, 7.2 Hz, 1H), 4.04-3.97 (m, 1H), 3.96 (s, 3H), 3.89 (q, J = 7.3 Hz, 2H), 3.13 (m, 1H), 2.02 (tt, J = 8.1, 4.6 Hz, 1H), 1.41 (d, J = 7.0 Hz, 3H), 1.37 (t, J = 7.3 Hz, 3H), 1.22 (dt, J = 6.3, 3.1 Hz, 2H), 0.97 (dt, J = 7.9, 3.3 Hz, 2H). | m/z: 571.2 [M + H]⁺ |
| | (R)-2-(4-cyclopropyl-6-methoxypyrimidin-5-yl)-4-(4-(1-ethyl-4- | | |
| | (trifluoromethyl)-1H-imidazol-2-yl)-3-fluorobenzyl)-6-methyl-6,7- | | |
| | dihydro-[1,2,4]triazolo[1,5-a]pyrimidin-5(4H)-one | | |
| 349 | 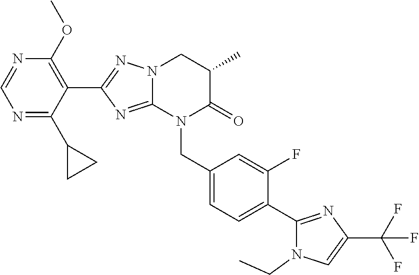 [SMILES: CCn1cc(C(F)(F)F)nc1-c1ccc(CN2C(=O)[C@@H](C)Cn3nc(-c4c(OC)ncnc4C4CC4)nc32)cc1F] | ¹H NMR (400 MHz, Chloroform-d) δ 8.62 (s, 1H), 7.49 (t, J = 7.5 Hz, 1H), 7.45- 7.38 (m, 3H), 5.18 (d, J = 1.8 Hz, 2H), 4.51 (dd, J = 13.0, 7.2 Hz, 1H), 4.04-3.97 (m, 1H), 3.96 (s, 3H), 3.89 (q, J = 7.3 Hz, 2H), 3.13 (m, 1H), 2.02 (tt, J = 8.1, 4.6 Hz, 1H), 1.41 (d, J = 7.0 Hz, 3H), 1.37 (t, J = 7.3 Hz, 3H), 1.22 (dt, J = 6.3, 3.1Hz, 2H), 0.97 (dd, J = 5.3, 2.8 Hz, 2H). | m/z: 571.2 [M + H]⁺ |
| | (S)-2-(4-cyclopropyl-6-methoxypyrimidin-5-yl)-4-(4-(1-ethyl-4- | | |
| | (trifluoromethyl)-1H-imidazol-2-yl)-3-fluorobenzyl)-6-methyl-6,7- | | |
| | dihydro-[1,2,4]triazolo[1,5-a]pyrimidin-5(4H)-one | | |
| 350 | 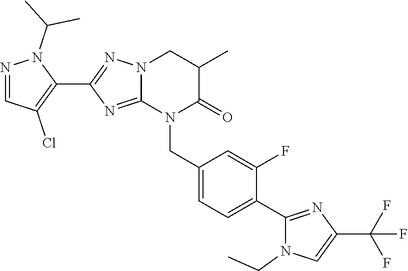 [SMILES: CCn1cc(C(F)(F)F)nc1-c1ccc(CN2C(=O)C(C)Cn3nc(-c4c(Cl)cnn4C(C)C)nc32)cc1F] | 1H NMR (400 MHz, Chloroform-d) δ 7.57-7.49 (m, 2H), 7.47-7.38 (m, 3H), 5.28-5.20 (m, 1H), 5.18 (s, 2H), 4.53 (dd, J = 12.9, 7.0 Hz, 1H), 4.08-3.97 (m, 1H), 3.90 (q, J = 7.2 Hz, 2H), 3.13 (dt, J = 10.4, 7.0 Hz, 1H), 1.50 (dd, J = 6.5, 3.7 Hz, 6H), 1.43 (d, J = 6.9 Hz, 3H), 1.38 (t, J = 7.2 Hz, 3H). | m/z: 565.2 [M + H]⁺ |
| | 2-(4-chloro-1-isopropyl-1H-pyrazol-5-yl)-4-(4-(1-ethyl-4- | | |
| | (trifluoromethyl)-1H-imidazol-2-yl)-3-fluorobenzyl)-6-methyl-6,7- | | |
| | dihydro-[1,2,4]triazolo[1,5-a]pyrimidin-5(4H)-one | | |
| 351 | 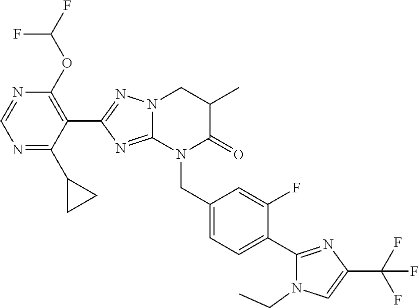 [SMILES: CCn1cc(C(F)(F)F)nc1-c1ccc(CN2C(=O)C(C)Cn3nc(-c4c(OC(F)F)ncnc4C4CC4)nc32)cc1F] | ¹H NMR (400 MHz, Chloroform-d) δ 8.64 (s, 1H), 7.52 (t, J = 71.6 Hz, 1H), 7.48 (dd, J = 15.5, 7.5 Hz, 2H), 7.38 (d, J = 9.0 Hz, 2H), 5.18 (s, 2H), 4.54 (dd, J = 12.8, 6.9 Hz, 1H), 4.03 (t, J = 11.9 Hz, 1H), 3.90 (q, J = 7.1 Hz, 2H), 3.22-3.12 (m, 1H), 2.22-2.15 (m, 1H), 1.44 (d, J = 6.6 Hz, 3H), 1.38 (t, J = 7.1 Hz, 3H), 1.30-1.27 (m, 2H), 1.08-1.03 (m, 2H). | m/z: 607.2 [M + H]⁺ |
| | 2-(4-cyclopropyl-6-(difluoromethoxy)pyrimidin-5-yl)-4-(4-(1-ethyl-4- | | |
| | (trifluoromethyl)-1H-imidazol-2-yl)-3-fluorobenzyl)-6-methyl-6,7- | | |
| | dihydro-[1,2,4]triazolo[1,5-a]pyrimidin-5(4H)-one | | |

### Example 352. 2-(4-chloro-1-isopropyl-1H-pyrazol-5-yl)-N-(4-(1-ethyl-4-(trifluoromethyl)-1H-imidazol-2-yl)phenyl)-5,5-dimethyl-4,5,6,7-tetrahydropyrazolo[1,5-α]pyridin-4-amine

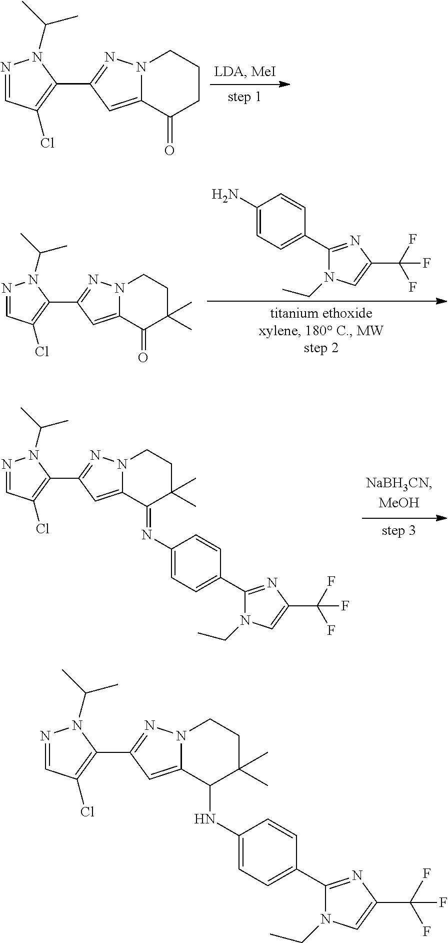 [SMILES: CC(C)n1ncc(Cl)c1-c1cc2n(n1)CCCC2=O ; CC(C)n1ncc(Cl)c1-c1cc2n(n1)CCC(C)(C)C2=O ; CCn1cc(C(F)(F)F)nc1-c1ccc(N)cc1 ; CCn1cc(C(F)(F)F)nc1-c1ccc(/N=C2/c3cc(-c4c(Cl)cnn4C(C)C)nn3CCC2(C)C)cc1 ; CCn1cc(C(F)(F)F)nc1-c1ccc(NC2c3cc(-c4c(Cl)cnn4C(C)C)nn3CCC2(C)C)cc1]

### Step 1. 2-(4-chloro-1-isopropyl-1H-pyrazol-5-yl)-5,5-dimethyl-6,7-dihydropyrazolo[1,5-α]pyridin-4(5H)-one

To a solution of 2-(4-chloro-1-isopropyl-1H-pyrazol-5-yl)-6,7-dihydropyrazolo[1,5-α]pyridin-4(5H)-one (300 mg, 1.08 mmol) in dry THF (10 mL) was added LDA (2 M in THF/hexane, 1.1 mL, 2.2 mmol) dropwise at −78° C. After stirring for 30 min, MeI (382 mg, 2.69 mmol) was added. The resulting mixture was allowed to warm to room temperature and stirred for 1 h then quenched with saturated aqueous NH₄Cl and extracted with EtOAc. The combined organic layers were washed with brine, dried over Na₂SO₄, concentrated under vacuum and purified by prep-TLC (eluting with 1/2 EtOAc/PE) to afford 2-(4-chloro-1-isopropyl-1H-pyrazol-5-yl)-5,5-dimethyl-6,7-dihydropyrazolo[1,5-α]pyridin-4(5H)-one (40 mg). MS (ESI) m/z 307.1 [M+H]⁺.

### Step 2. (E)-2-(4-chloro-1-isopropyl-1H-pyrazol-5-yl)-N-(4-(1-ethyl-4-(trifluoromethyl)-1H-imidazol-2-yl)phenyl)-5,5-dimethyl-6,7-dihydropyrazolo[1,5-α]pyridin-4(5H)-imine

A solution of 2-(4-chloro-1-isopropyl-1H-pyrazol-5-yl)-5,5-dimethyl-6,7-dihydropyrazolo[1,5-α]pyridin-4(5H)-one (60 mg, 0.195 mmol), 4-(1-ethyl-4-(trifluoromethyl)-1H-imidazol-2-yl)aniline (150 mg, 0.59 mmol) and titanium ethoxide (89 mg, 0.39 mmol) in dry xylene (2 mL) was heated at 180° C. in microwave reactor for 4 h. The solvent was removed under vacuum and the residue was purified by prep-TLC (elting with 1/1 PE/EtOAc) to afford (E)-2-(4-chloro-1-isopropyl-1H-pyrazol-5-yl)-N-(4-(1-ethyl-4-(trifluoromethyl)-1H-imidazol-2-yl)phenyl)-5,5-dimethyl-6,7-dihydropyrazolo[1,5-α]pyridin-4(5H)-imine (20 mg). MS (ESI) m/z 544.2 [M+H]⁺.

### Step 3. 2-(4-chloro-1-isopropyl-1H-pyrazol-5-yl)-N-(4-(1-ethyl-4-(trifluoromethyl)-1H-imidazol-2-yl)phenyl)-5,5-dimethyl-4,5,6,7-tetrahydropyrazolo[1,5-α]pyridin-4-amine

Example 352 2-(4-chloro-1-isopropyl-1H-pyrazol-5-yl)-N-(4-(1-ethyl-4-(trifluoromethyl)-1H-imidazol-2-yl)phenyl)-5,5-dimethyl-4,5,6,7-tetrahydropyrazolo[1,5-α]pyridin-4-amine was synthesized according to step 7 of Example 237. ¹H NMR (400 MHz, Chloroform-d) δ 7.46 (s, 1H), 7.43 (d, J=8.3 Hz, 2H), 7.34-7.31 (m, 1H), 6.82 (d, J=8.4 Hz, 2H), 6.47 (s, 1H), 5.18 (m, 1H), 4.52 (s, 1H), 4.30 (m, 1H), 4.18 (m, 1H), 4.09-4.04 (m, 2H), 2.12 (m, 1H), 2.05-1.97 (m, 1H), 1.47-1.42 (m, 9H), 1.22 (s, 3H), 1.04 (s, 3H). MS (ESI) m/z 546.2 [M+H]⁺.

### Example 353. 2-(4-chloro-1-isopropyl-1H-pyrazol-5-yl)-4-(4-(1-ethyl-4-(trifluoromethyl)-1H-imidazol-2-yl)-3-fluorobenzyl)-4,5,6,7-tetrahydropyrazolo[1,5-α]pyrimidin-6-amine

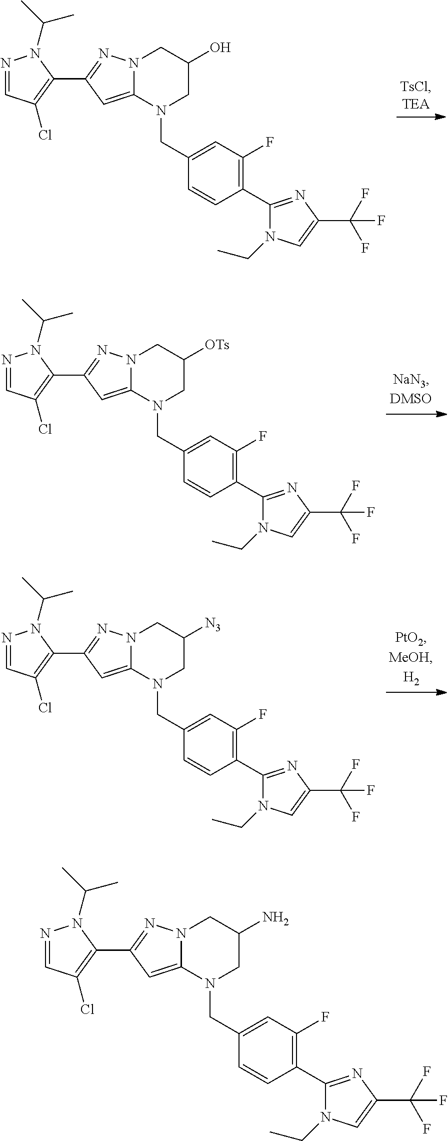 [SMILES: CCn1cc(C(F)(F)F)nc1-c1ccc(CN2CC(O)Cn3nc(-c4c(Cl)cnn4C(C)C)cc32)cc1F ; CCn1cc(C(F)(F)F)nc1-c1ccc(CN2CC(OS(=O)(=O)c3ccc(C)cc3)Cn3nc(-c4c(Cl)cnn4C(C)C)cc32)cc1F ; CCn1cc(C(F)(F)F)nc1-c1ccc(CN2CC(N=[N+]=[N-])Cn3nc(-c4c(Cl)cnn4C(C)C)cc32)cc1F ; CCn1cc(C(F)(F)F)nc1-c1ccc(CN2CC(N)Cn3nc(-c4c(Cl)cnn4C(C)C)cc32)cc1F]

### Step 1. 2-(4-chloro-1-isopropyl-1H-pyrazol-5-yl)-4-(4-(1-ethyl-4-(trifluoromethyl)-1H-imidazol-2-yl)-3-fluorobenzyl)-4,5,6,7-tetrahydropyrazolo[1,5-α]pyrimidin-6-yl 4-methylbenzenesulfonate

To a stirred solution of 2-(4-chloro-1-isopropyl-1H-pyrazol-5-yl)-4-(4-(1-ethyl-4-(trifluoromethyl)-1H-imidazol-2-yl)-3-fluorobenzyl)-4,5,6,7-tetrahydropyrazolo[1,5-α]pyrimidin-6-ol (420 mg, 0.76 mmol) in dry DCM (20 mL) was added TEA (154 mg, 1.52 mmol), tosyl chloride (290.6 mg, 1.52 mmol) and DMAP (18.6 mg, 0.15 mmol). The resulting mixture was stirred at room temperature for 3 h then diluted with water and extracted with EtOAc. The combined organic layers were washed with brine, dried over Na₂SO₄ and concentrated under vacuum to give crude 2-(4-chloro-1-isopropyl-1H-pyrazol-5-yl)-4-(4-(1-ethyl-4-(trifluoromethyl)-1H-imidazol-2-yl)-3-fluorobenzyl)-4,5,6,7-tetrahydropyrazolo[1,5-α]pyrimidin-6-yl 4-methylbenzenesulfonate (505 mg). MS (ESI) m/z 706.2 [M+H]⁺.

### Step 2. 6-azido-2-(4-chloro-1-isopropyl-1H-pyrazol-5-yl)-4-(4-(1-ethyl-4-(trifluoromethyl)-1H-imidazol-2-yl)-3-fluorobenzyl)-4,5,6,7-tetrahydropyrazolo[1,5-α]pyrimidine

A mixture of 2-(4-chloro-1-isopropyl-1H-pyrazol-5-yl)-4-(4-(1-ethyl-4-(trifluoromethyl)-1H-imidazol-2-yl)-3-fluorobenzyl)-4,5,6,7-tetrahydropyrazolo[1,5-α]pyrimidin-6-yl 4-methylbenzenesulfonate (500 mg, 0.71 mmol) and NaN₃ (92.2 mg, 1.42 mmol) in DMF (5 mL) was heated at 100° C. for 2 h. After cooling to room temperature, the resulting mixture was diluted with water (50 mL) and extracted with EtOAc (50 mL×3). The combined organic layers were washed with brine, dried over Na₂SO₄, concentrated under vacuum and purified by column chromatography on silica gel (eluting with 1/1 PE/EtOAc) to give 6-azido-2-(4-chloro-1-isopropyl-1H-pyrazol-5-yl)-4-(4-(1-ethyl-4-(trifluoromethyl)-1H-imidazol-2-yl)-3-fluorobenzyl)-4,5,6,7-tetrahydropyrazolo[1,5-α]pyrimidine (200 mg). MS (ESI) m/z 577.2 [M+H]⁺.

### Step 3. 2-(4-chloro-1-isopropyl-1H-pyrazol-5-yl)-4-(4-(1-ethyl-4-(trifluoromethyl)-1H-imidazol-2-yl)-3-fluorobenzyl)-4,5,6,7-tetrahydropyrazolo[1,5-α]pyrimidin-6-amine

A mixture of 6-azido-2-(4-chloro-1-isopropyl-1H-pyrazol-5-yl)-4-(4-(1-ethyl-4-(trifluoromethyl)-1H-imidazol-2-yl)-3-fluorobenzyl)-4,5,6,7-tetrahydropyrazolo[1,5-α]pyrimidine (200 mg, 0.35 mmol) and PtO2 (7.9 mg, 0.035 mmol) in MeOH (5 mL) was stirred at room temperature under an atmosphere of H₂ for 2 h. The resulting mixture was filtered through celite and concentrated under vacuum. The residue was purified by prep-TLC (eluting with 1/10 MeOH/DCM) to give Example 353 2-(4-chloro-1-isopropyl-1H-pyrazol-5-yl)-4-(4-(1-ethyl-4-(trifluoromethyl)-1H-imidazol-2-yl)-3-fluorobenzyl)-4,5,6,7-tetrahydropyrazolo[1,5-α]pyrimidin-6-amine (37.9 mg). ¹H NMR (400 MHz, Chloroform-d) δ 7.54 (t, J=7.5 Hz, 1H), 7.46 (d, J=3.1 Hz, 1H), 7.41 (s, 1H), 7.25 (d, J=7.0 Hz, 1H), 7.19 (d, J=10.5 Hz, 1H), 5.80 (d, J=2.9 Hz, 1H), 5.24 (m, 1H), 4.40 (s, 2H), 4.33 (dd, J=12.6, 4.5 Hz, 1H), 3.93 (m, 3H), 3.66 (s, 1H), 3.27 (d, J=11.1 Hz, 1H), 3.06 (dd, J=11.0, 6.6 Hz, 1H), 2.47 (s, 2H), 1.44 (dd, J=6.5, 3.2 Hz, 6H), 1.41-1.36 (m, 3H). MS (ESI) m/z 551.1 [M+H]⁺.

### Example 354. 2-(4-chloro-1-isopropyl-1H-pyrazol-5-yl)-4-((5-(1-ethyl-4-(trifluoromethyl)-1H-imidazol-2-yl)pyrimidin-2-yl)methyl)-6,7-dihydropyrazolo[1,5-α]pyrimidin-5(4H)-one

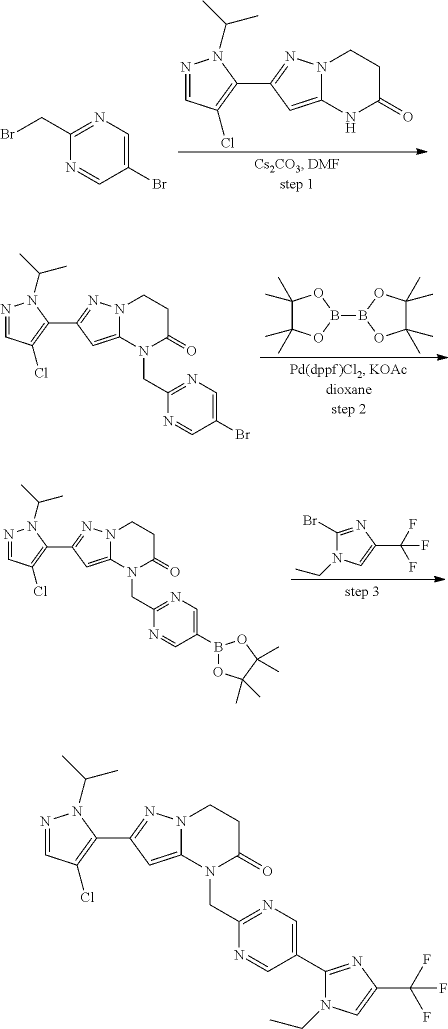

### Step 1. 44(5-bromopyrimidin-2-yl)methyl)-2-(4-chloro-1-isopropyl-1H-pyrazol-5-yl)-6,7-dihydropyrazolo[1,5-α]pyrimidin-5(4H)-one

A mixture of 2-(4-chloro-1-isopropyl-1H-pyrazol-5-yl)-6,7-dihydropyrazolo[1,5-α]pyrimidin-5(4H)-one (279 mg, 1.0 mmol), 5-bromo-2-(bromomethyl)pyrimidine (252 mg, 1.0 mmol) and Cs₂CO₃ (978 mg, 3.0 mmol) in DMF (10 mL) was stirred at room temperature for 2 h. The reaction mixture was diluted with water (50 mL) and extracted with EtOAc (30 mL×3). The combined organic phases were concentrated and purified on a Biotage Isolera One (C₁₈ column, eluting with 10% to 90% MeCN/H₂O) to afford 4-((5-bromopyrimidin-2-yl)methyl)-2-(4-chloro-1-isopropyl-1H-pyrazol-5-yl)-6,7-dihydropyrazolo[1,5-α]pyrimidin-5(4H)-one (110 mg). MS (ESI) m/z 450.0, 452.0 [M+H]⁺.

### Step 2: 2-(4-chloro-1-isopropyl-1H-pyrazol-5-yl)-4-((5-(4,4,5,5-tetramethyl-1,3,2-dioxaborolan-2-yl)pyrimidin-2-yl) methyl)-6,7-dihydropyrazolo[1,5-α]pyrimidin-5(4H)-one

A mixture of 4-((5-bromopyrimidin-2-yl)methyl)-2-(4-chloro-1-isopropyl-1H-pyrazol-5-yl)-6,7-dihydropyrazolo[1,5-α]pyrimidin-5(4H)-one (110 mg, 0.24 mmol), 4,4,4′,4′,5,5,5′,5′-octamethyl-2,2′-bi(1,3,2-dioxaborolane) (122 mg, 0.48 mmol), KOAc (47 mg, 0.48 mmol) and Pd(dppf)Cl₂ (18 mg, 0.024 mmol) in dioxane (80 mL) was heated at 100° C. for 4 h under an atmosphere of N₂. The resulting mixture was filtered through celite, concentrated and purified on a Biotage Isolera One (C₁₈ column, eluting with 10% to 90% MeCN/H₂O) to afford 2-(4-chloro-1-isopropyl-1H-pyrazol-5-yl)-4-((5-(4,4,5,5-tetramethyl-1,3,2-dioxaborolan-2-yl)pyrimidin-2-yl)methyl)-6,7-dihydropyrazolo[1,5-α]pyrimidin-5(4H)-one (89 mg). MS (ESI) m/z 498.2 [M+H]⁺.

### Step 3: 2-(4-chloro-1-isopropyl-1H-pyrazol-5-yl)-4-((5-(1-ethyl-4-(trifluoromethyl)-1H-imidazol-2-yl)pyrimidin-2-yl) methyl)-6,7-dihydropyrazolo[1,5-α]pyrimidin-5(4H)-one

A mixture of 2-(4-chloro-1-isopropyl-1H-pyrazol-5-yl)-4-((5-(4,4,5,5-tetramethyl-1,3,2-dioxaborolan-2-yl)pyrimidin-2-yl)methyl)-6,7-dihydropyrazolo[1,5-α]pyrimidin-5(4H)-one (150 mg, 0.3 mmol), 2-bromo-1-ethyl-4-(trifluoromethyl)-1H-imidazole (145 mg, 0.6 mmol), Pd(dppf)Cl₂ (22 mg, 0.03 mmol) and K₃PO₄ (191 mg, 0.9 mmol) in a mixture of dioxane (5 mL) and H₂O (0.5 mL) was heated at 110° C. overnight under an atmosphere of N₂. The mixture was filtered through celite. The filtrate was concentrated and purified by prep-TLC (eluting with 1/1 PE/EtOAc) to afford Example 354 2-(4-chloro-1-isopropyl-1H-pyrazol-5-yl)-4-((5-(1-ethyl-4-(trifluoromethyl)-1H-imidazol-2-yl)pyrimidin-2-yl)methyl)-6,7-dihydropyrazolo[1,5-α]pyrimidin-5(4H)-one (42 mg). ¹H NMR (400 MHz, Chloroform-d) δ 8.92 (s, 2H), 7.44 (s, 2H), 6.01 (s, 1H), 5.29 (s, 2H), 5.19 (m, 1H), 4.49 (t, J=7.0 Hz, 2H), 4.06 (q, J=7.2 Hz, 2H), 3.13 (t, J=6.9 Hz, 2H), 1.46 (m, 9H). MS (ESI) m/z 534.1 [M+H]⁺.

### Example 355. N-(4-(1-ethyl-4-(trifluoromethyl)-1H-imidazol-2-yl)-3-fluorophenyl)-2-(2-isopropylphenyl)-4,5,6,7-tetrahydro-2H-indazol-4-amine and Example 356 N-(4-(1-ethyl-4-(trifluoromethyl)-1H-imidazol-2-yl)-3-fluorophenyl)-2-(2-isopropylphenyl)-N-methyl-4,5,6,7-tetrahydro-2H-indazol-4-amine

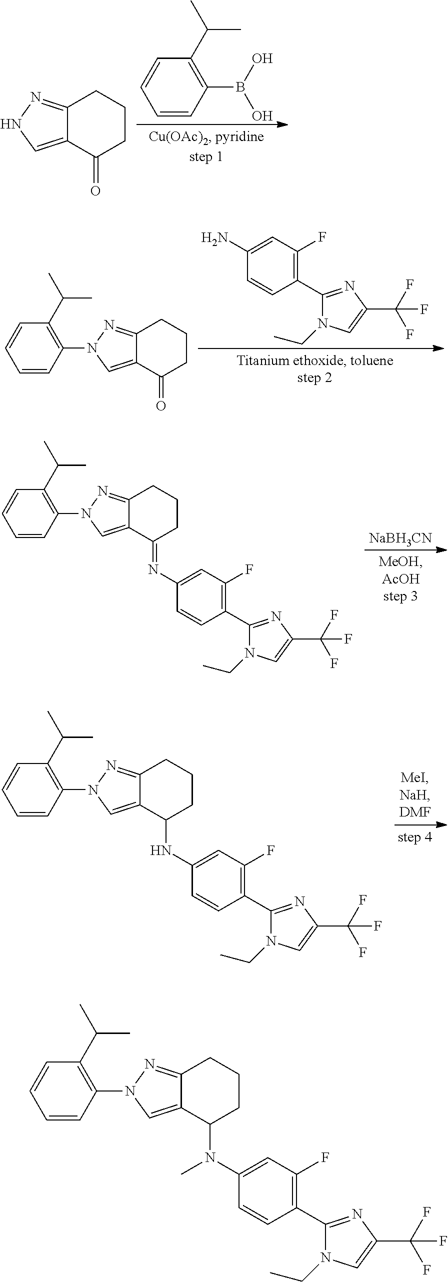

### Step 1. 2-(2-isopropylphenyl)-2,5,6,7-tetrahydro-4H-indazol-4-one

To a solution of (2-isopropylphenyl)boronic acid (900.0 mg, 5.5 mmol) in DCM (15 mL) was added pyridine (348.0 mg, 4.4 mmol), Cu(OAc)₂ (795.0 mg, 4.4 mmol) and 2,5,6,7-tetrahydro-4H-indazol-4-one (300.0 mg, 2.2 mmol). The mixture was stirred at room temperature for 12 h under an atmosphere of 02. The resulting mixture was filtered through celite and the filtrate was concentrated under vacuum. The crude product was purified on a Biotage Isolera One (C₁₈ column, eluting with 10% to 90% MeCN/H₂O) to afford 2-(2-isopropylphenyl)-2,5,6,7-tetrahydro-4H-indazol-4-one (400 mg). ¹H NMR (400 MHz, Chloroform-d) δ 7.98 (s, 1H), 7.46-7.42 (m, 2H), 7.28-7.23 (m, 2H), 2.93 (t, J=6.3 Hz, 2H), 2.86 (m, 1H), 2.56 (dd, J=7.1, 5.6 Hz, 2H), 2.20 (tt, J=7.2, 5.7 Hz, 2H), 1.17 (d, J=6.9 Hz, 6H).

### Step 2 and step 3. N-(4-(1-ethyl-4-(trifluoromethyl)-1H-imidazol-2-yl)-3-fluorophenyl)-2-(2-isopropylphenyl)-4,5,6,7-tetrahydro-211-indazol-4-amine

Example 355 N-(4-(1-ethyl-4-(trifluoromethyl)-1H-imidazol-2-yl)-3-fluorophenyl)-2-(2-isopropylphenyl)-4,5,6,7-tetrahydro-2H-indazol-4-amine was prepared from 2-(2-isopropylphenyl)-2,5,6,7-tetrahydro-4H-indazol-4-one and 4-(1-ethyl-4-(trifluoromethyl)-1H-imidazol-2-yl)-3-fluoroaniline following step 6 and step 7 of Example 237. ¹H NMR (400 MHz, Chloroform-d) δ 7.45 (d, J=4.9 Hz, 1H), 7.43-7.37 (m, 2H), 7.37-7.29 (m, 2H), 7.23 (dd, J=4.6, 1.9 Hz, 2H), 6.54 (d, J=7.1 Hz, 1H), 6.45 (d, J=12.7 Hz, 1H), 4.76-4.68 (m, 1H), 3.92 (q, J=7.3 Hz, 2H), 2.93 (m, 1H), 2.80 (t, J=6.2 Hz, 2H), 2.22-2.02 (m, 2H), 1.84 (m, 2H), 1.39 (t, J=7.3 Hz, 3H), 1.17 (dd, J=6.8, 4.3 Hz, 6H). MS (ESI) m/z 512.3 [M+H]⁺.

### Step 4. N-(4-(1-ethyl-4-(trifluoromethyl)-1H-imidazol-2-yl)-3-fluorophenyl)-2-(2-isopropylphenyl)-N-methyl-4,5,6,7-tetrahydro-211-indazol-4-amine

Example 356 N-(4-(1-ethyl-4-(trifluoromethyl)-1H-imidazol-2-yl)-3-fluorophenyl)-2-(2-isopropylphenyl)-N-methyl-4,5,6,7-tetrahydro-2H-indazol-4-amine was synthesized from N-(4-(1-ethyl-4-(trifluoromethyl)-1H-imidazol-2-yl)-3-fluorophenyl)-2-(2-isopropylphenyl)-4,5,6,7-tetrahydro-2H-indazol-4-amine according to Example 258. ¹H NMR (400 MHz, Chloroform-d) δ 7.42-7.33 (m, 4H), 7.27 (s, 1H), 7.24 (dd, J=4.5, 2.5 Hz, 2H), 6.71 (dd, J=8.8, 2.5 Hz, 1H), 6.57 (dd, J=14.2, 2.4 Hz, 1H), 5.12 (dd, J=9.4, 5.1 Hz, 1H), 3.92 (q, J=7.3 Hz, 2H), 2.94 (m, 1H), 2.89-2.71 (m, 5H), 2.19-2.05 (m, 2H), 1.91-1.76 (m, 2H), 1.38 (t, J=7.3 Hz, 3H), 1.17 (dd, J=10.8, 6.9 Hz, 6H). MS (ESI) m/z 526.3 [M+H]⁺.

### Example 357. 2-(4-chloro-1-isopropyl-1H-pyrazol-5-yl)-N-(4-(1-ethyl-4-(trifluoromethyl)-1H-imidazol-2-yl)phenyl)-5,6,7,8-tetrahydro-4H-pyrazolo[1,5-α]azepin-4-amine

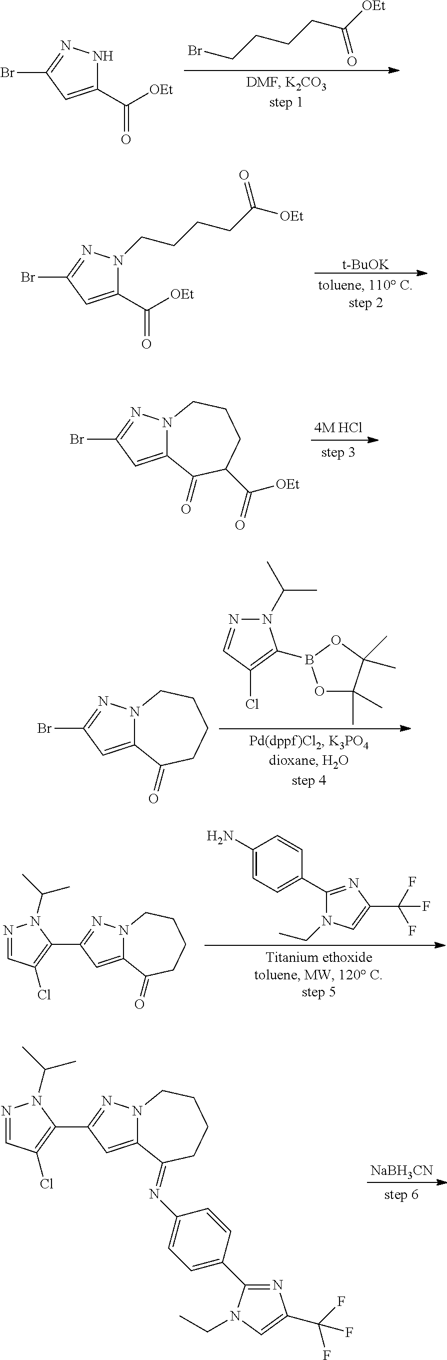

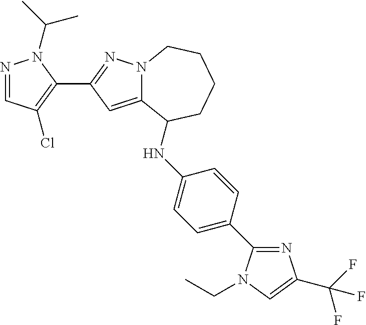 [SMILES: CCn1cc(C(F)(F)F)nc1-c1ccc(NC2CCCCn3nc(-c4c(Cl)cnn4C(C)C)cc32)cc1]

### Step 1 to step 6. 2-(4-chloro-1-isopropyl-1H-pyrazol-5-yl)-N-(4-(1-ethyl-4-(trifluoromethyl)-1H-imidazol-2-yl)phenyl)-5,6,7,8-tetrahydro-4H-pyrazolo[1,5-a]azepin-4-amine

Example 357 2-(4-chloro-1-isopropyl-1H-pyrazol-5-yl)-N-(4-(1-ethyl-4-(trifluoromethyl)-1H-imidazol-2-yl)phenyl)-5,6,7,8-tetrahydro-4H-pyrazolo[1,5-α]azepin-4-amine was prepared from ethyl 3-bromo-1H-pyrazole-5-carboxylate (synthesized in step 1 of Example 237) and ethyl 5-bromopentanoate following step 2 to step 7 of Example 237. ¹H NMR (400 MHz, Chloroform-d) δ 7.46 (s, 1H), 7.38 (d, J=8.7 Hz, 2H), 7.30 (d, J=1.1 Hz, 1H), 6.69 (d, J=8.7 Hz, 2H), 6.51 (s, 1H), 5.20 (m, 1H), 4.61 (dd, J=19.2, 6.1 Hz, 2H), 4.26 (dd, J=13.7, 10.5 Hz, 1H), 4.02 (q, J=7.3 Hz, 2H), 2.21-2.13 (m, 2H), 2.00 (dd, J=13.7, 7.1 Hz, 2H), 1.83-1.71 (m, 2H), 1.46 (d, J=6.6 Hz, 3H), 1.44-1.38 (m, 6H). MS (ESI) m/z 532.2 [M+H]⁺.

### Example 358. 2-(4-chloro-1-isopropyl-1H-pyrazol-5-yl)-4-(4-(1-ethyl-4-(trifluoromethyl)-1H-imidazol-2-yl)phenoxy)-5,6,7,8-tetrahydro-4H-pyrazolo[1,5-α]azepine

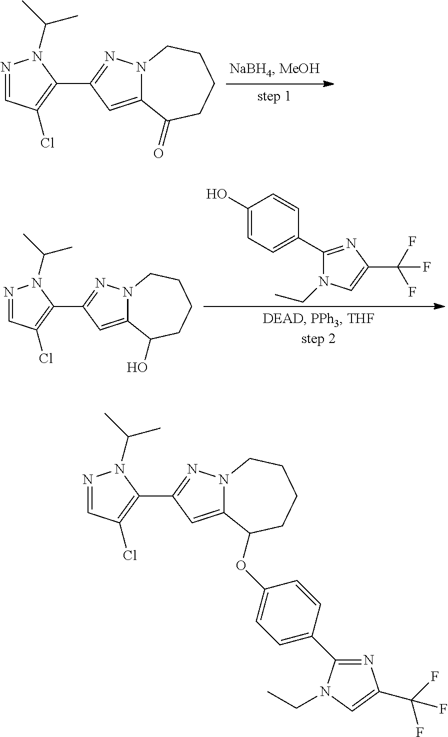 [SMILES: CC(C)n1ncc(Cl)c1-c1cc2n(n1)CCCCC2=O ; CC(C)n1ncc(Cl)c1-c1cc2n(n1)CCCCC2O ; CCn1cc(C(F)(F)F)nc1-c1ccc(O)cc1 ; CCn1cc(C(F)(F)F)nc1-c1ccc(OC2CCCCn3nc(-c4c(Cl)cnn4C(C)C)cc32)cc1]

### Step 1 and step 2. 2-(4-chloro-1-isopropyl-1H-pyrazol-5-yl)-4-(4-(1-ethyl-4-(trifluoromethyl)-1H-imidazol-2-yl)phenoxy)-5,6,7,8-tetrahydro-4H-pyrazolo[1,5-α]azepine

Example 358 2-(4-chloro-1-isopropyl-1H-pyrazol-5-yl)-4-(4-(1-ethyl-4-(trifluoromethyl)-1H-imidazol-2-yl)phenoxy)-5,6,7,8-tetrahydro-4H-pyrazolo[1,5-α]azepine was prepared from 2-(4-chloro-1-isopropyl-1H-pyrazol-5-yl)-5,6,7,8-tetrahydro-4H-pyrazolo[1,5-α]azepin-4-one (synthesized in step 4 of Example 357) following Example 261. ¹H NMR (400 MHz, Methanol-d₄) δ 7.73 (q, J=1.3 Hz, 1H), 7.59-7.44 (m, 3H), 7.28-7.18 (m, 2H), 6.71 (s, 1H), 5.84-5.77 (m, 1H), 5.11 (m, 1H), 4.58-4.45 (m, 2H), 4.07 (q, J=7.3 Hz, 2H), 2.33 (m, 2H), 2.02 (d, J=15.5 Hz, 3H), 1.83 (d, J=11.2 Hz, 1H), 1.37 (dt, J=19.6, 7.1 Hz, 9H). MS (ESI) m/z 533.2 [M+H]⁺.

### Example 359. 2-(4-chloro-1-isopropyl-1H-pyrazol-5-yl)-4-(4-(1-ethyl-4-(trifluoromethyl)-1H-imidazol-2-yl)benzyl)-4,5,6,7-tetrahydropyrazolo[1,5-α]pyrazine and Example 360 2-(4-chloro-1-isopropyl-1H-pyrazol-5-yl)-4-(4-(1-ethyl-4-(trifluoromethyl)-1H-imidazol-2-yl)benzyl)-5-methyl-4,5,6,7-tetrahydropyrazolo[1,5-α]pyrazine

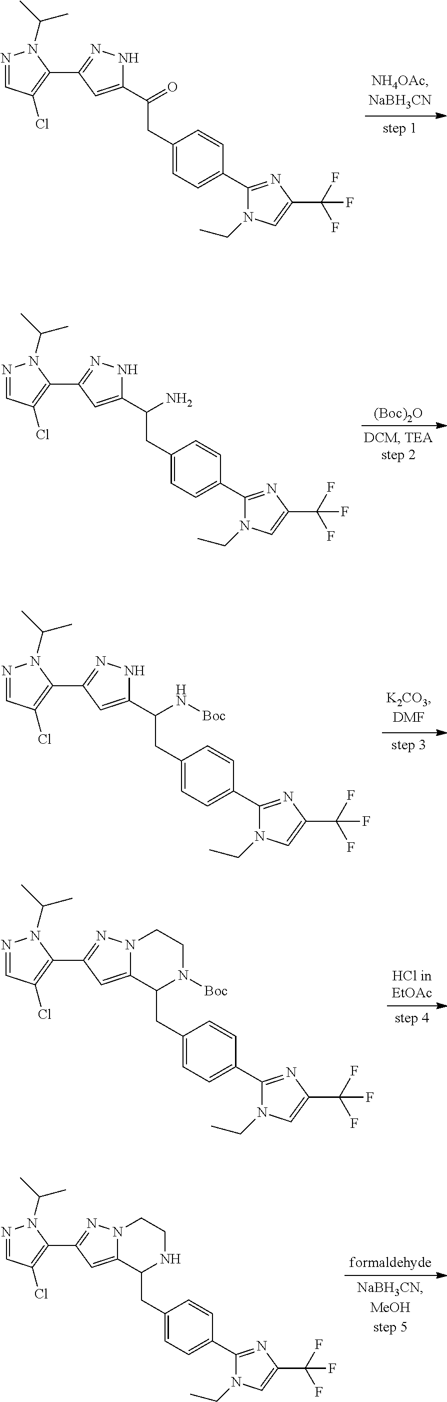 [SMILES: "(Boc)2O" ; CCn1cc(C(F)(F)F)nc1-c1ccc(CC(=O)c2cc(-c3c(Cl)cnn3C(C)C)n[nH]2)cc1 ; CCn1cc(C(F)(F)F)nc1-c1ccc(CC(N)c2cc(-c3c(Cl)cnn3C(C)C)n[nH]2)cc1 ; CCn1cc(C(F)(F)F)nc1-c1ccc(CC(NC(=O)OC(C)(C)C)c2cc(-c3c(Cl)cnn3C(C)C)n[nH]2)cc1 ; CCn1cc(C(F)(F)F)nc1-c1ccc(CC2c3cc(-c4c(Cl)cnn4C(C)C)nn3CCN2C(=O)OC(C)(C)C)cc1 ; CCn1cc(C(F)(F)F)nc1-c1ccc(CC2NCCn3nc(-c4c(Cl)cnn4C(C)C)cc32)cc1]

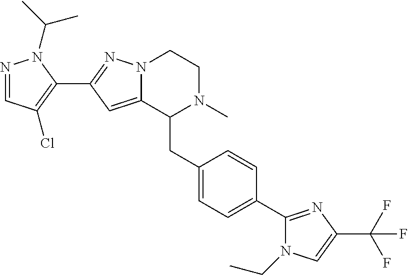 [SMILES: CCn1cc(C(F)(F)F)nc1-c1ccc(CC2c3cc(-c4c(Cl)cnn4C(C)C)nn3CCN2C)cc1]

### Step 1. 1-(4′-chloro-2′-isopropyl-1H,2′H-[3,3′-bipyrazol]-5-yl)-2-(4-(1-ethyl-4-(trifluoromethyl)-1H-imidazol-2-yl) phenyl)ethan-1-amine

To a solution of 1-(4′-chloro-2′-isopropyl-1H,2′H-[3,3′-bipyrazol]-5-yl)-2-(4-(1-ethyl-4-(trifluoromethyl)-1H-imidazol-2-yl)phenyl)ethan-1-one (580.0 mg, 1.2 mmol) in MeOH (10 mL) was added NH40Ac (454.8 mg, 5.9 mmol) and NaBH₃CN (368.0 mg, 5.9 mmol). The resulting mixture was heated at reflux for 4 h then quenched with water, concentrated and purified on a Biotage Isolera One (C₁₈ column, eluting with 10% to 90% MeCN/H₂O, containing 0.1% TFA) to afford 1-(4′-chloro-2′-isopropyl-1H,2′H-[3,3′-bipyrazol]-5-yl)-2-(4-(1-ethyl-4-(trifluoromethyl)-1H-imidazol-2-yl)phenyl)ethan-1-amine (306.0 mg). MS (ESI) m/z 492.1 [M+H]⁺.

### Step 2. tert-butyl(1-(4′-chloro-2′-isopropyl-1H,2′H-[3,3′-bipyrazol]-5-yl)-2-(4-(1-ethyl-4-(trifluoromethyl)-1H-imidazol-2-yl)phenyl)ethyl)carbamate

To a solution of 1-(4′-chloro-2′-isopropyl-1H,2′H-[3,3′-bipyrazol]-5-yl)-2-(4-(1-ethyl-4-(trifluoromethyl)-1H-imidazol-2-yl)phenyl)ethan-1-amine (901.0 mg, 1.8 mmol) in DCM (10 mL) was added TEA (554.5 mg, 5.5 mmol) and (Boc)20 (797.9 mg, 3.7 mmol). The mixture was stirred at room temperature for 1 h. The solvent was removed under vacuum and the crude product was purified on a Biotage Isolera One (C₁₈ column, eluting with 10% to 90% MeCN/H₂O) to give tert-butyl(1-(4′-chloro-2′-isopropyl-1H,2′H-[3,3′-bipyrazol]-5-yl)-2-(4-(1-ethyl-4-(trifluoromethyl)-1H-imidazol-2-yl)phenyl)ethyl)carbamate (591.0 mg). MS (ESI) m/z 592.2 [M+H]⁺.

### Step 3. tert-butyl 2-(4-chloro-1-isopropyl-1H-pyrazol-5-yl)-4-(4-(1-ethyl-4-(trifluoromethyl)-1H-imidazol-2-yl)benzyl)-6,7-dihydropyrazolo[1,5-α]pyrazine-5(4H)-carboxylate

To a solution of tert-butyl (1-(4′-chloro-2′-isopropyl-1H,2′H-[3,3′-bipyrazol]-5-yl)-2-(4-(1-ethyl-4-(trifluoromethyl)-1H-imidazol-2-yl)phenyl)ethyl)carbamate (591.2 mg, 1.0 mmol) in DMF (5 mL) was added 1,2-dibromoethane (557.6 mg, 3.0 mmol) and K₂CO₃ (276.0 mg 2.0 mmol). The mixture was heated in a sealed tube at 100° C. overnight. The resulting mixture was diluted with water (20 mL) and extracted with EtOAc (3×30 mL). The combined organic phases were washed with brine, dried over anhydrous Na₂SO₄ and concentrated under vacuum to afford crude tert-butyl 2-(4-chloro-1-isopropyl-1H-pyrazol-5-yl)-4-(4-(1-ethyl-4-(trifluoromethyl)-1H-imidazol-2-yl)benzyl)-6,7-dihydropyrazolo[1,5-α]pyrazine-5(4H)-carboxylate (300 mg) which was used directly in next step without further purification. MS (ESI) m/z 618.2 [M+H]⁺.

### Step 4. 2-(4-chloro-1-isopropyl-1H-pyrazol-5-yl)-4-(4-(1-ethyl-4-(trifluoromethyl)-1H-imidazol-2-yl)benzyl)-4,5,6,7-tetrahydropyrazolo[1,5-α]pyrazine

To a solution of tert-butyl 2-(4-chloro-1-isopropyl-1H-pyrazol-5-yl)-4-(4-(1-ethyl-4-(trifluoromethyl)-1H-imidazol-2-yl)benzyl)-6,7-dihydropyrazolo[1,5-α]pyrazine-5(4H)-carboxylate (300 mg, 0.48 mmol) in DCM (2 mL) was added TFA (2 mL). The mixture was stirred at room temperature for 1 h then concentrated under vacuum. The crude product was purified on a Biotage Isolera One (C₁₈ column, eluting with 10% to 90% MeCN/H₂O, containing 0.1% trifluoroacetic acid) and neutralized with 1M aqueous NaOH to give Example 359 2-(4-chloro-1-isopropyl-1H-pyrazol-5-yl)-4-(4-(1-ethyl-4-(trifluoromethyl)-1H-imidazol-2-yl)benzyl)-4,5,6,7-tetrahydropyrazolo[1,5-α]pyrazine (100 mg).

### Step 5. 2-(4-chloro-1-isopropyl-1H-pyrazol-5-yl)-4-(4-(1-ethyl-4-(trifluoromethyl)-1H-imidazol-2-yl)benzyl)-5-methyl-4,5,6,7-tetrahydropyrazolo[1,5-α]pyrazine

To a solution of 2-(4-chloro-1-isopropyl-1H-pyrazol-5-yl)-4-(4-(1-ethyl-4-(trifluoromethyl)-1H-imidazol-2-yl)benzyl)-4,5,6,7-tetrahydropyrazolo[1,5-α]pyrazine (100 mg, 0.19 mmol) in MeOH (3 mL) was added paraformaldehyde (90 mg, 1.0 mmol) and 1 drop of acetic acid at room temperature. The mixture was stirred at room temperature for 30 minutes then NaBH₃CN (62.8 mg, 1.0 mmol) was added. The resulting mixture was stirred at room temperature for 1 h then quenched with water. The solvent was removed under vacuum and the crude product purified on a Biotage Isolera One (C₁₈ column, eluting with 10% to 90% MeCN/H₂O, containing 0.1% trifluoroacetic acid) and neutralized with 1M aqueous NaOH to give Example 360 2-(4-chloro-1-isopropyl-1H-pyrazol-5-yl)-4-(4-(1-ethyl-4-(trifluoromethyl)-1H-imidazol-2-yl)benzyl)-5-methyl-4,5,6,7-tetrahydropyrazolo[1,5-α]pyrazine (6.4 mg).

Table 27. The compounds listed in Table 27 were synthesized according to Example 359 and Example 360 using the appropriate commercially available reagents and/or intermediates described above.

TABLE 27

| Example | | | |
|---|---|---|---|
| number | Structure | ¹H NMR (δ ppm) | MS (ESI) |
| 359 | 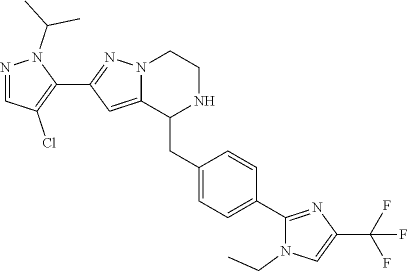 [SMILES: CCn1cc(C(F)(F)F)nc1-c1ccc(CC2NCCn3nc(-c4c(Cl)cnn4C(C)C)cc32)cc1] | ¹H NMR (400 MHz, Chloroform-d) δ 7.57 (d, J = 8.2 Hz, 2H), 7.51 (s, 1H), 7.43-7.36 (m, 3H), 6.47 (s, 1H), 5.21 (m, 1H), 4.35 (dd, J = 9.1, 4.4 Hz, 1H), 4.19 (dt, J = 10.6, 4.8 Hz, 2H), 4.09 (q, J = 7.3 Hz, 2H), 3.46-3.33 (m, 2H), 3.26-3.16 (m, 1H), 3.05 (dd, J = 13.6, 9.1 Hz, 1H), 1.49-1.44 (m, 9H). | m/z: 518.2 [M + H]⁺ |
| | 2-(4-chloro-1-isopropyl-1H-pyrazol-5-yl)-4-(4-(1-ethyl-4- | | |
| | (trifluoromethyl)-1H-imidazol-2-yl)benzyl)-4,5,6,7- | | |
| | tetrahydropyrazolo[1,5-a]pyrazine | | |
| 360 | 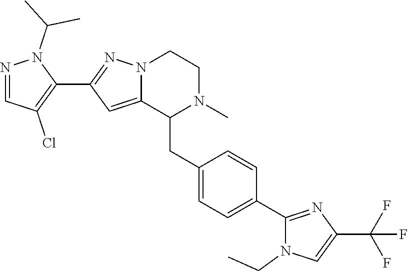 [SMILES: CCn1cc(C(F)(F)F)nc1-c1ccc(CC2c3cc(-c4c(Cl)cnn4C(C)C)nn3CCN2C)cc1] | ¹H NMR (400 MHz, Chloroform-d) δ 7.50-7.45 (m, 3H), 7.35 (s, 1H), 7.30 (d, J = 8.0 Hz, 2H), 6.15 (s, 1H), 5.16 (m, 1H), 4.25-4.10 (m, 2H), 4.08-3.97 (m, 3H), 3.33-3.24 (m, 2H), 3.12 (dd, J = 14.2, 6.9 Hz, 1H), 3.00 (m, 1H), 2.62 (s, 3H), 1.43 (q, J = 6.9 Hz, 9H). | m/z: 532.2 [M + H]⁺ |
| | 2-(4-chloro-1-isopropyl-1H-pyrazol-5-yl)-4-(4-(1-ethyl-4- | | |
| | (trifluoromethyl)-1H-imidazol-2-yl)benzyl)-5-methyl-4,5,6,7- | | |
| | tetrahydropyrazolo[1,5-a]pyrazine | | |
| 361 | 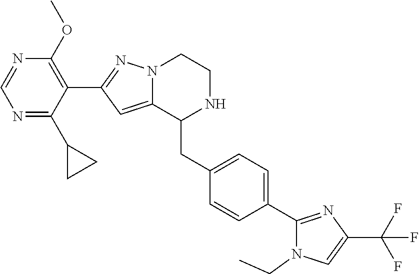 [SMILES: CCn1cc(C(F)(F)F)nc1-c1ccc(CC2NCCn3nc(-c4c(OC)ncnc4C4CC4)cc32)cc1] | ¹H NMR (400 MHz, Chloroform-d) δ 8.57 (s, 1H), 7.56 (d, J = 7.9 Hz, 2H), 7.44 (d, J = 7.7 Hz, 2H), 7.40-7.35 (m, 1H), 6.22 (s, 1H), 4.46 (s, 1H), 4.29 (s, 2H), 4.09 (q, J = 7.3 Hz, 2H), 3.95 (s, 3H), 3.53-3.11 (m, 4H), 2.30-2.23 (m, 1H), 1.45 (t, J = 7.3 Hz, 3H), 1.18 (d, J = 4.6 Hz, 2H), 0.98-0.92 (m, 2H). | m/z: 524.2 [M + H]⁺ |
| | 2-(4-cyclopropyl-6-methoxypyrimidin-5-yl)-4-(4-(1-ethyl-4- | | |
| | (trifluoromethyl)-1H-imidazol-2-yl)benzyl)-4,5,6,7- | | |
| | tetrahydropyrazolo[1,5-a]pyrazine | | |
| 362 | 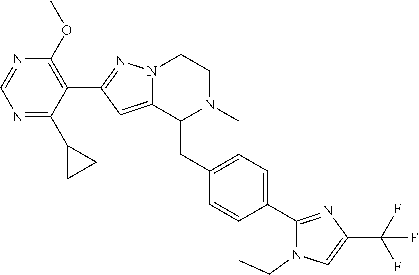 | ¹H NMR (400 MHz, Chloroform-d) δ 8.55 (s, 1H), 7.51 (d, J = 7.1 Hz, 2H), 7.35 (s, 3H), 5.90 (s, 1H), 4.32 (s, 2H), 4.05 (q, J = 7.2 Hz, 3H), 3.91 (s, 3H), 3.21 (s, 4H), 2.66 (d, J = 57.2 Hz, 3H), 2.15 (s, 1H), 1.42 (t, J = 7.2 Hz, 3H), 1.18-1.14 (m, 2H), 0.92 (dd, J = 7.8, 3.1 Hz, 2H). | m/z: 583.3 [M + H]⁺ |
| | 2-(4-cyclopropyl-6-methoxypyrimidin-5-yl)-4-(4-(1-ethyl-4- | | |
| | (trifluoromethyl)-1H-imidazol-2-yl)benzyl)-5-methyl-4,5,6,7- | | |
| | tetrahydropyrazolo[1,5-a]pyrazine | | |

### Example 363. Enantioselective synthesis of (R)-2-(4-cyclopropyl-6-methoxypyrimidin-5-yl)-4-(1-(4-(1-isopropyl-4-(trifluoromethyl)-1H-imidazol-2-yl)phenyl)ethyl)-6,7-dihydropyrazolo[1,5-α]pyrimidin-5(4H)-one

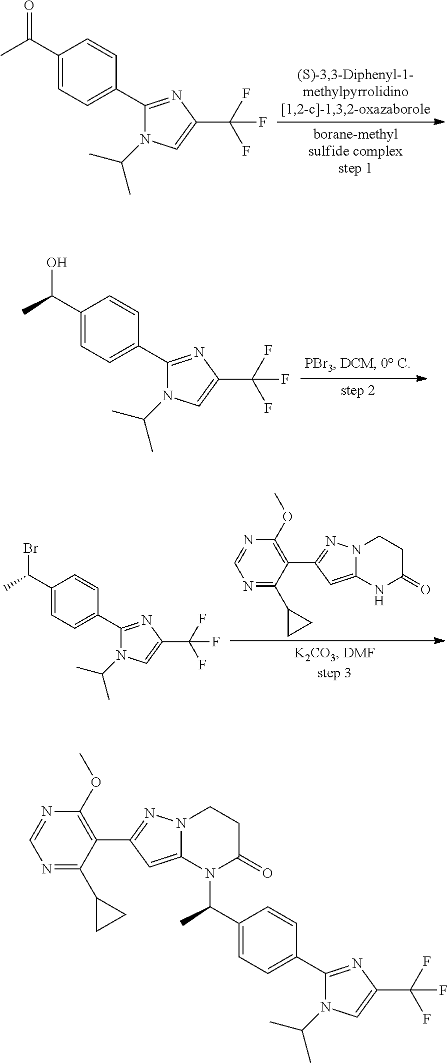 [SMILES: CC(=O)c1ccc(-c2nc(C(F)(F)F)cn2C(C)C)cc1 ; CC(C)n1cc(C(F)(F)F)nc1-c1ccc([C@@H](C)O)cc1 ; CC(C)n1cc(C(F)(F)F)nc1-c1ccc([C@H](C)Br)cc1 ; COc1ncnc(C2CC2)c1-c1cc2n(n1)CCC(=O)N2 ; COc1ncnc(C2CC2)c1-c1cc2n(n1)CCC(=O)N2[C@H](C)c1ccc(-c2nc(C(F)(F)F)cn2C(C)C)cc1]

### Step 1. (R)-1-(4-(1-isopropyl-4-(trifluoromethyl)-1H-imidazol-2-yl)phenyl)ethan-1-ol

To a solution of borane-methyl sulfide complex (2 M in THF, 0.5 mL, 1 mmol) in dry THF (1 mL) was added (S)-3,3-Diphenyl-1-methylpyrrolidino[1,2-c]-1,3,2-oxazaborole (1 M in toluene, 0.5 mL, 0.5 mmol) at −20° C. The solution was stirred at the same temperature for 30 min then 1-(4-(1-isopropyl-4-(trifluoromethyl)-1H-imidazol-2-yl)phenyl)ethan-1-one (100 mg, 0.34 mmol, prepared in Intermediate BB-54) in dry THF (1 mL) was added. The resulting mixture was stirred at −20° C. for 2 h then warmed to room temperature, quenched with water (10 mL) and extracted with EtOAc (10 mL×3). The combined organic layers were dried over anhydrous Na₂SO₄, concentrated under vacuum and purified by prep-TLC (eluting with 1/3 EtOAc/PE) to afford (R)-1-(4-(1-isopropyl-4-(trifluoromethyl)-1H-imidazol-2-yl)phenyl)ethan-1-ol (60 mg, 98% ee). The ee value was determined by chiral HPLC using a Superchiral S-OJ (0.46×15 cm) column and eluting with 90/10 hexane/ethanol containing 0.5% diethylamine at a flow of 0.9 mL/min at room temperature. MS (ESI) m/z 299.1 [M+H]⁺.

### Step 2. (S)-2-(4-(1-bromoethyl)phenyl)-1-isopropyl-4-(trifluoromethyl)-1H-imidazole

To a solution of (R)-1-(4-(1-isopropyl-4-(trifluoromethyl)-1H-imidazol-2-yl)phenyl)ethan-1-ol (60 mg, 0.2 mmol) in dry DCM (3 mL) was added PBr₃ (108 mg, 0.4 mmol) at 0° C. The resulting mixture was stirred at same temperature for 2 h then quenched with saturated aqueous NaHCO₃ (5 mL) and extracted with DCM (5 mL×3). The combined organic layers were dried over anhydrous Na₂SO₄, concentrated under vacuum and purified by prep-TLC (eluting with 1/5 EtOAc/PE) to afford (S)-2-(4-(1-bromoethyl)phenyl)-1-isopropyl-4-(trifluoromethyl)-1H-imidazole (30 mg). MS (ESI) m/z 361.0, 363.0 [M+H]⁺.

### Step 3. (R)-2-(4-cyclopropyl-6-methoxypyrimidin-5-yl)-4-(1-(4-(1-isopropyl-4-(trifltmromethyl)-1H-imidazol-2-yl)phenyl)ethyl)-6,7-dihydropyrazolo[1,5-α]pyrimidin-5(4H)-one

A mixture of (S)-2-(4-(1-bromoethyl)phenyl)-1-isopropyl-4-(trifluoromethyl)-1H-imidazole (30 mg, 0.083 mmol), 2-(4-cyclopropyl-6-methoxypyrimidin-5-yl)-6,7-dihydropyrazolo[1,5-α]pyrimidin-5(4H)-one (24 mg, 0.083 mmol, prepared according to step 1 of Example 77) and K₂CO₃ (23 mg, 0.17 mmol) in dry DMF (1 mL) was stirred at room temperature for 16 h. The resulting mixture was diluted with water (5 mL) and extracted with EtOAc (5 mL×3). The combined organic layers were washed with brine, dried over anhydrous Na₂SO₄, concentrated under vacuum and purified by prep-TLC (eluting with 1/1 EtOAc/PE) to afford Example 363 (R)-2-(4-cyclopropyl-6-methoxypyrimidin-5-yl)-4-(1-(4-(1-isopropyl-4-(trifluoromethyl)-1H-imidazol-2-yl)phenyl)ethyl)-6,7-dihydropyrazolo[1,5-α]pyrimidin-5(4H)-one (10 mg, 75% ee). The ee value was determined by chiral HPLC using a UniChiral OD-5H (0.46×15 cm) column and eluting with 80/20 hexane/ethanol containing 0.5% diethylamine at a flow of 1 mL/min at room temperature. ¹H NMR (400 MHz, Chloroform-d) δ 8.52 (s, 1H), 7.54 (d, J=8.3 Hz, 2H), 7.46 (d, J=8.1 Hz, 2H), 7.41 (s, 1H), 6.26 (q, J=7.1 Hz, 1H), 5.53 (s, 1H), 4.54 (m, 1H), 4.49-4.38 (m, 2H), 3.86 (s, 3H), 3.21-3.01 (m, 2H), 2.11 (m, 1H), 1.83 (d, J=7.2 Hz, 3H), 1.45 (d, J=6.7 Hz, 6H), 1.12 (dt, J=5.8, 3.2 Hz, 2H), 0.87 (m, 2H). MS (ESI) m/z 566.2 [M+H]⁺.

### Example 364. 2-(4-cyclopropyl-6-(difluoromethoxy)pyrimidin-5-yl)-4-(4-(1-ethyl-4-(trifluoromethyl)-1H-imidazol-2-yl)benzyl)-6,7-dihydro-[1,2,4]triazolo[1,5-α]pyrimidin-5(4H)-one

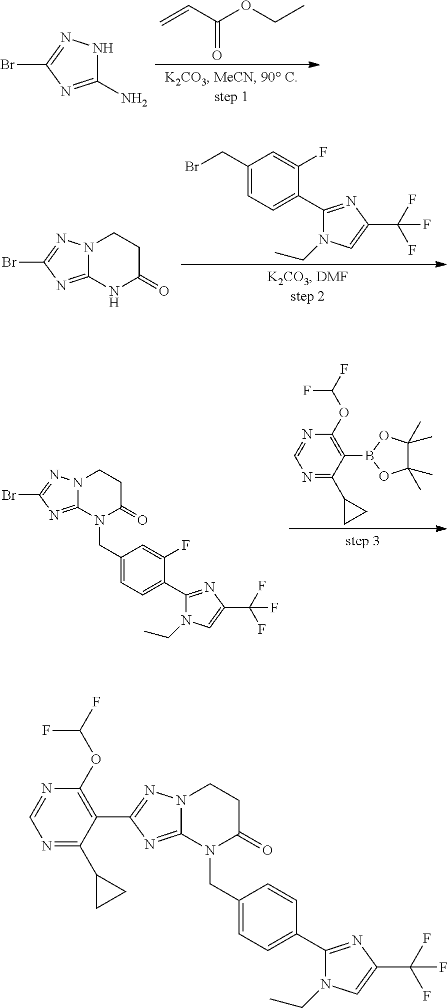

### Step 1 to step 3. 2-(4-cyclopropyl-6-(difluoromethoxy)pyrimidin-5-yl)-4-(4-(1-ethyl-4-(trifluoromethyl)-1H-imidazol-2-yl)benzyl)-6,7-dihydro-[1,2,4]triazolo[1,5-α]pyrimidin-5(4H)-one

Example 364 2-(4-cyclopropyl-6-(difluoromethoxy)pyrimidin-5-yl)-4-(4-(1-ethyl-4-(trifluoromethyl)-1H-imidazol-2-yl)benzyl)-6,7-dihydro-[1,2,4]triazolo[1,5-α]pyrimidin-5(4H)-one was synthesized from 3-bromo-1H-1,2,4-triazol-5-amine (prepared in Example 337) and ethyl acrylate following step 1 to step 3 of Example 347.

Table 28. The compounds listed in Table 28 were synthesized according to Example 364 using the appropriate commercially available reagents and/or intermediates described above.

TABLE 28

| Example | | | |
|---|---|---|---|
| number | Structure | ¹H NMR (δ ppm) | MS (ESI) |
| 364 | 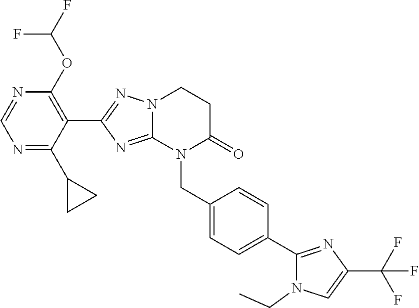 [SMILES: CCn1cc(C(F)(F)F)nc1-c1ccc(CN2C(=O)CCn3nc(-c4c(OC(F)F)ncnc4C4CC4)nc32)cc1] | ¹H NMR (400 MHz, Chloroform-d) δ 8.64 (s, 1H), 7.68 (d, J = 7.7 Hz, 2H), 7.57- 7.49 (m, 3H), 7.36 (s, 1H), 5.20 (s, 2H), 4.41 (t, J = 7.3 Hz, 2H), 4.05 (q, J = 7.3 Hz, 2H), 3.14 (t, J = 1.2 Hz, 2H), 2.23 (d, J = 4.3 Hz, 1H), 1.43 (t, J = 7.2 Hz, 3H), 1.27 (s, 2H), 1.05 (d, J = 4.6 Hz, 2H). | m/z: 575.2 [M + H]⁺ |
| | 2-(4-cyclopropyl-6-(difluoromethoxy)pyrimidin-5-yl)-4-(4-(1-ethyl-4- | | |
| | (trifluoromethyl)-1H-imidazol-2-yl)benzyl)-6,7-dihydro- | | |
| | [1,2,4]triazolo[1,5-a]pyrimidin-5(4H)-one | | |
| 365 | 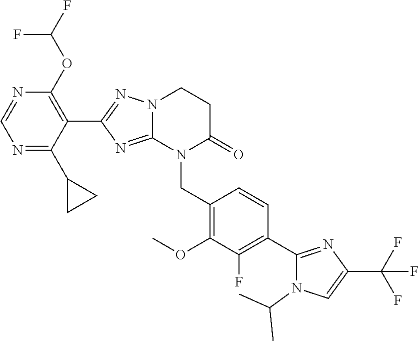 [SMILES: COc1c(CN2C(=O)CCn3nc(-c4c(OC(F)F)ncnc4C4CC4)nc32)ccc(-c2nc(C(F)(F)F)cn2C(C)C)c1F] | ¹H NMR (400 MHz, Chloroform-d) δ 8.62 (s, 1H), 7.49 (t, J = 71.6 Hz, 1H), 7.43 (s, 1H), 7.15 (d, J = 22.1 Hz, 2H), 5.28 (s, 2H), 4.47 (s, 2H), 4.24 (s, 1H), 3.99 (d, J = 1.6 Hz, 3H), 3.17 (s, 2H), 2.19 (d, J = 19.9 Hz, 1H), 1.41 (d, J = 6.9 Hz, 6H), 1.26 (s, 2H), 1.04 (s, 2H). | m/z: 637.3 [M + H]⁺ |
| | 2-(4-cyclopropyl-6-(difluoromethoxy)pyrimidin-5-yl)-4-(3-fluoro-4-(1- | | |
| | isopropyl-4-(trifluoromethyl)-1H-imidazol-2-yl)-2-methoxybenzyl)- | | |
| | 6,7-dihydro-[1,2,4]triazolo[1,5-a]pyrimidin-5(4H)-one | | |
| 366 | 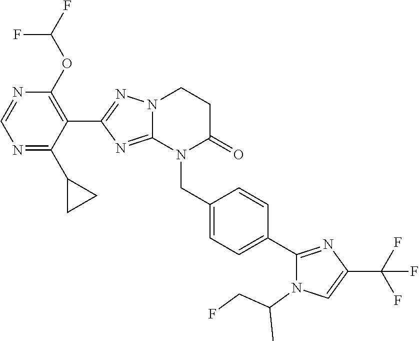 [SMILES: CC(CF)n1cc(C(F)(F)F)nc1-c1ccc(CN2C(=O)CCn3nc(-c4c(OC(F)F)ncnc4C4CC4)nc32)cc1] | ¹H NMR (400 MHz, Chloroform-d) δ 8.64 (d, J = 2.5 Hz, 1H), 7.68 (dd, J = 8.1, 2.5 Hz, 2H), 7.55 (t, J = 71.6 Hz, 1H), 7.48 (d, J = 5.6 Hz, 3H), 5.20 (d, J = 2.5 Hz, 2H), 4.71-4.53 (m, 2H), 4.42 (m, 3H), 3.13 (td, J = 7.4, 2.5 Hz, 2H), 2.28-2.18 (m, 1H), 1.50 (d, J = 6.9 Hz, 3H), 1.27 (d, J = 3.7 Hz, 2H), 1.06 (dq, J = 7.2, 3.2 Hz, 2H). | m/z: 607.2 [M + H]⁺ |
| | 2-(4-cyclopropyl-6-(difluoromethoxy)pyrimidin-5-yl)-4-(4-(1-(1- | | |
| | fluoropropan-2-yl)-4-(trifluoromethyl)-1H-imidazol-2-yl)benzyl)-6,7- | | |
| | dihydro-[1,2,4]triazolo[1,5-a]pyrimidin-5(4H)-one | | |
| 367 | 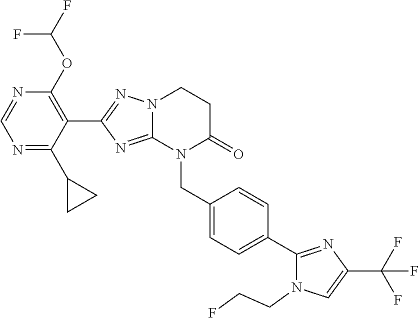 [SMILES: O=C1CCn2nc(-c3c(OC(F)F)ncnc3C3CC3)nc2N1Cc1ccc(-c2nc(C(F)(F)F)cn2CCF)cc1] | ¹H NMR (400 MHz, Chloroform-d) δ 8.64 (s, 1H), 7.67 (d, J = 7.2 Hz, 2H), 7.54 (t, J = 71.6 Hz, 1H), 7.52-7.43 (m, 3H), 5.19 (s, 2H), 4.70 (s, 1H), 4.58 (s, 1H), 4.40 (t, J = 6.6 Hz, 2H), 4.31 (s, 1H), 4.24 (s, 1H), 3.12 (t, J = 6.6 Hz, 2H), 2.23 (d, J = 2.6 Hz, 1H), 1.27 (s, 2H), 1.10-0.97 (m, 2H). | m/z: 593.2 [M + H]⁺ |
| | 2-(4-cyclopropyl-6-(difluoromethoxy)pyrimidin-5-yl)-4-(4-(1-(2- | | |
| | fluoroethyl)-4-(trifluoromethyl)-1H-imidazol-2-yl)benzyl)-6,7- | | |
| | dihydro-[1,2,4]triazolo[1,5-a]pyrimidin-5(4H)-one | | |
| 368 | 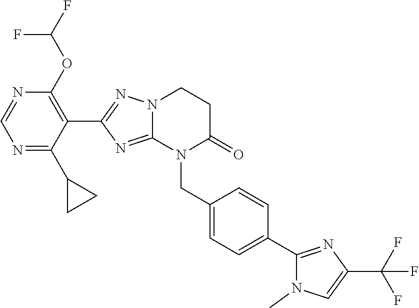 [SMILES: Cn1cc(C(F)(F)F)nc1-c1ccc(CN2C(=O)CCn3nc(-c4c(OC(F)F)ncnc4C4CC4)nc32)cc1] | ¹H NMR (400 MHz, DMSO- d₆) δ 8.75 (s, 1H), 7.89 (s, 1H), 7.78 (t, J = 71.6 Hz, 1H), 7.63 (d, J = 8.0 Hz, 2H), 7.53 (d, J = 7.8 Hz, 2H), 5.09 (s, 2H), 4.45 (t, J = 7.3 Hz, 2H), 3.75 (s, 3H), 3.19-3.14 (m, 2H), 2.22 (s, 1H), 1.10 (s, 2H), 0.99 (d, J = 7.7 Hz, 2H). | m/z: 561.3 [M + H]⁺ |
| | 2-(4-cyclopropyl-6-(difluoromethoxy)pyrimidin-5-yl)-4-(4-(1-methyl- | | |
| | 4-(trifluoromethyl)-1H-imidazol-2-yl)benzyl)-6,7-dihydro- | | |
| | [1,2,4]triazolo[1,5-a]pyrimidin-5(4H)-one | | |
| 369 | 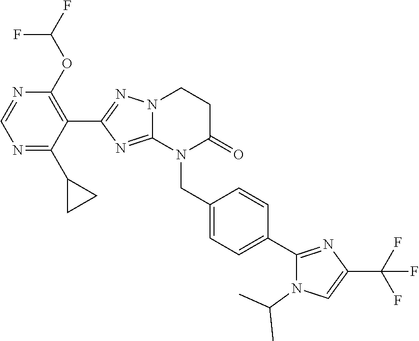 [SMILES: CC(C)n1cc(C(F)(F)F)nc1-c1ccc(CN2C(=O)CCn3nc(-c4c(OC(F)F)ncnc4C4CC4)nc32)cc1] | ¹H NMR (400 MHz, Chloroform-d) δ 8.63 (s, 1H), 7.67 (d, J = 8.1 Hz, 2H), 7.54 (t, J = 71.6 Hz, 1H), 7.48 (d, J = 8.1 Hz, 2H), 7.40 (s, 1H), 5.19 (s, 2H), 4.53 (m, 1H), 4.40 (t, J = 7.3 Hz, 2H), 3.12 (t, J = 7.3 Hz, 2H), 2.23 (tt, J = 8.2, 4.6 Hz, 1H), 1.43 (d, J = 6.7 Hz, 6H), 1.29-1.25 (m, 2H), 1.04 (dq, J = 7.1, 3.8 Hz, 2H). | m/z: 589.1 [M + H]⁺ |
| | 2-(4-cyclopropyl-6-(difluoromethoxy)pyrimidin-5-yl)-4-(4-(1-isopropyl- | | |
| | 4-(trifluoromethyl)-1H-imidazol-2-yl)benzyl)-6,7-dihydro- | | |
| | [1,2,4]triazolo[1,5-a]pyrimidin-5(4H)-one | | |
| 370 | 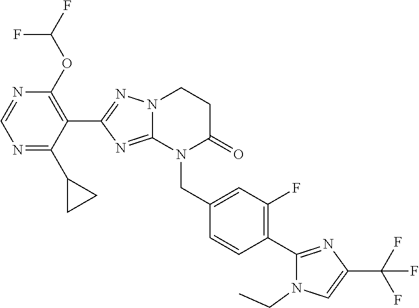 [SMILES: CCn1cc(C(F)(F)F)nc1-c1ccc(CN2C(=O)CCn3nc(-c4c(OC(F)F)ncnc4C4CC4)nc32)cc1F] | ¹H NMR (400 MHz, Chloroform-d) δ 8.64 (s, 1H), 7.74-7.31 (m, 5H), 5.18 (s, 2H), 4.43 (s, 2H), 3.90 (s, 2H), 3.15 (s, 2H), 2.20 (s, 1H), 1.38 (s, 3H), 1.28 (s, 2H), 1.06 (s, 2H). | m/z: 593.3 [M + H]⁺ |
| | 2-(4-cyclopropyl-6-(difluoromethoxy)pyrimidin-5-yl)-4-(4-(1-ethyl-4- | | |
| | (trifluoromethyl)-1H-imidazol-2-yl)-3-fluorobenzyl)-6,7-dihydro- | | |
| | [1,2,4]triazolo[1,5-a]pyrimidin-5(4H)-one | | |

### Example 371. 2-(4-chloro-1-isopropyl-1H-pyrazol-5-yl)-4-(2,2,2-trifluoro-1-(3-fluoro-4-(1-isopropyl-4-(trifluoromethyl)-1H-imidazol-2-yl)phenyl)ethyl)-4,5,6,7-tetrahydropyrazolo[1,5-α]pyrimidine

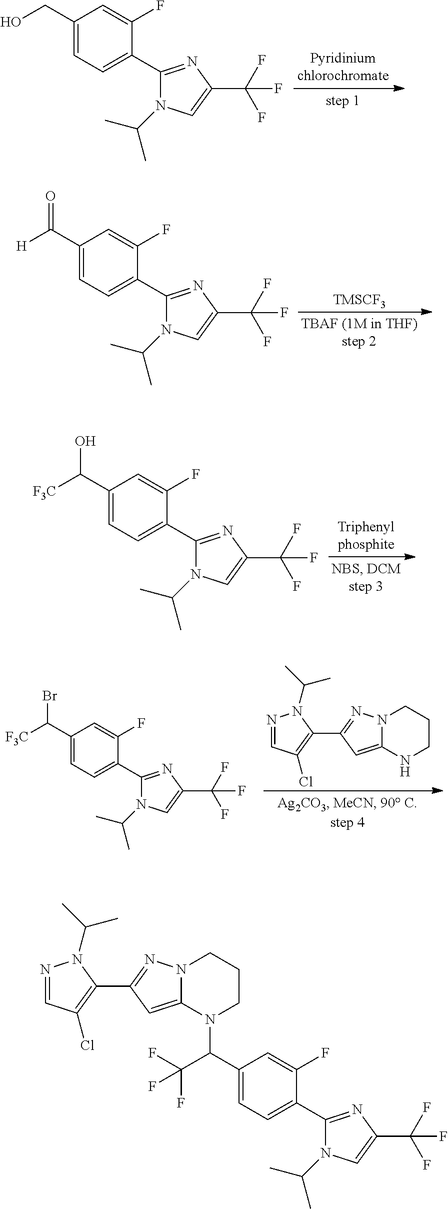

### Step 1. 3-fluoro-4-(1-isopropyl-4-(trifluoromethyl)-1H-imidazol-2-yl)benzaldehyde

To a solution of (3-fluoro-4-(1-isopropyl-4-(trifluoromethyl)-1H-imidazol-2-yl)phenyl)methanol (1 g, 3.3 mmol, synthesized in step 2 of Intermediate BB-6) in dry DCM (10 mL) at 0° C. was added pyridinium chlorochromate (815 mg, 3.8 mmol). The resulting mixture was stirred at room temperature for 4 h. The reaction was quenched with saturated aqueous sodium thiosulfate (10 mL) and extracted with DCM (10 mL×2). The combined organic phases were washed with brine, dried over anhydrous Na₂SO₄, concentrated and purified by column chromatography on silica gel (eluting with 1/10 EtOAc/PE) to give 3-fluoro-4-(1-isopropyl-4-(trifluoromethyl)-1H-imidazol-2-yl)benzaldehyde (930 mg). MS (ESI) m/z 301.1 [M+H]⁺.

### Step 2. 2,2,2-trifluoro-1-(3-fluoro-4-(1-isopropyl-4-(trifluoromethyl)-1H-imidazol-2-yl)phenyl)ethan-1-ol

To a stirred solution of 3-fluoro-4-(1-isopropyl-4-(trifluoromethyl)-1H-imidazol-2-yl)benzaldehyde (450 mg, 1.5 mmol) in dry THF (6 mL) at 0° C. was added TMSCF₃ (415 mg, 2.9 mmol) and TBAF (1 M in THF, 0.06 mL). The resulting mixture was stirred at room temperature for 2 h. The reaction mixture was then acidified with aqueous HCl (3 M, 15 mL) and extracted with EtOAc (30 mL×2). The combined organic phases were washed with brine, dried over anhydrous Na₂SO₄ and concentrated to give a white solid. The solid was recrystallized in EtOAc/hexane and dried under vacuum to give 2,2,2-trifluoro-1-(3-fluoro-4-(1-isopropyl-4-(trifluoromethyl)-1H-imidazol-2-yl)phenyl)ethan-1-ol (294 mg). MS (ESI) m/z 371.0 [M+H]⁺.

### Step 3. 2-(4-(1-bromo-2,2,2-trifluoroethyl)-2-fluorophenyl)-1-isopropyl-4-(trifluoromethyl)-1H-imidazole

To a solution of 2,2,2-trifluoro-1-(3-fluoro-4-(1-isopropyl-4-(trifluoromethyl)-1H-imidazol-2-yl)phenyl)ethan-1-ol (200 mg, 0.54 mmol) and triphenyl phosphite (167 mg, 0.54 mmol) at 0° C. under an atmosphere of N₂ was added NBS (141 mg, 0.79 mmol). The resulting mixture was stirred at room temperature for 12 h then diluted with water (20 mL) and extracted with EtOAc (25 mL×2). The combined organic phases were washed with brine, dried over anhydrous Na₂SO₄, concentrated and purified by column chromatography on silica gel (eluting with 1/20 EtOAc/PE) to give 2-(4-(1-bromo-2,2,2-trifluoroethyl)-2-fluorophenyl)-1-isopropyl-4-(trifluoromethyl)-1H-imidazole (178 mg). MS (ESI) m/z 433.0, 435.0 [M+H]⁺.

### Step 4. 2-(4-chloro-1-isopropyl-1H-pyrazol-5-yl)-4-(2,2,2-trifluoro-1-(3-fluoro-4-(1-isopropyl-4-(trifluoromethyl)-1H-imidazol-2-yl)phenyl)ethyl)-4,5,6,7-tetrahydropyrazolo[1,5-α]pyrimidine

A mixture of 2-(4-chloro-1-isopropyl-1H-pyrazol-5-yl)-4,5,6,7-tetrahydropyrazolo[1,5-α]pyrimidine (40 mg, 0.15 mmol, prepared according to Example 52), 2-(4-(1-bromo-2,2,2-trifluoroethyl)-2-fluorophenyl)-1-isopropyl-4-(trifluoromethyl)-1H-imidazole (78 mg, 0.18 mmol) and Ag2CO3 (82.7 mg, 0.3 mmol) in dry MeCN (5 mL) was heated at 80° C. overnight. The reaction was filtered through celite. The filtrate was concentrated and purified by prep-TLC (eluting with 1/1 EtOAc/PE) to give Example 371 2-(4-chloro-1-isopropyl-1H-pyrazol-5-yl)-4-(2,2,2-trifluoro-1-(3-fluoro-4-(1-isopropyl-4-(trifluoromethyl)-1H-imidazol-2-yl)phenyl)ethyl)-4,5,6,7-tetrahydropyrazolo[1,5-α]pyrimidine (3.3 mg). ¹H NMR (400 MHz, Chloroform-d) δ 7.65 (t, J=7.7 Hz, 1H), 7.48 (d, J=9.7 Hz, 2H), 7.40 (d, J=7.6 Hz, 1H), 7.32 (d, J=10.5 Hz, 1H), 5.95 (s, 1H), 5.34-5.19 (m, 2H), 4.28 (m, 1H), 4.16 (td, J=7.9, 4.4 Hz, 2H), 3.32 (t, J=9.7 Hz, 1H), 3.03 (ddd, J=11.4, 7.2, 3.4 Hz, 1H), 2.20 (dd, J=13.1, 7.1 Hz, 1H), 2.09 (s, 1H), 1.50-1.46 (m, 6H), 1.46-1.42 (m, 6H). MS (ESI) m/z 618.1 [M+H]⁺.

### Intermediate 34. 4-(difluoromethoxy)-6-methoxy-5-(4,4,5,5-tetramethyl-1,3,2-dioxaborolan-2-yl)pyrimidine

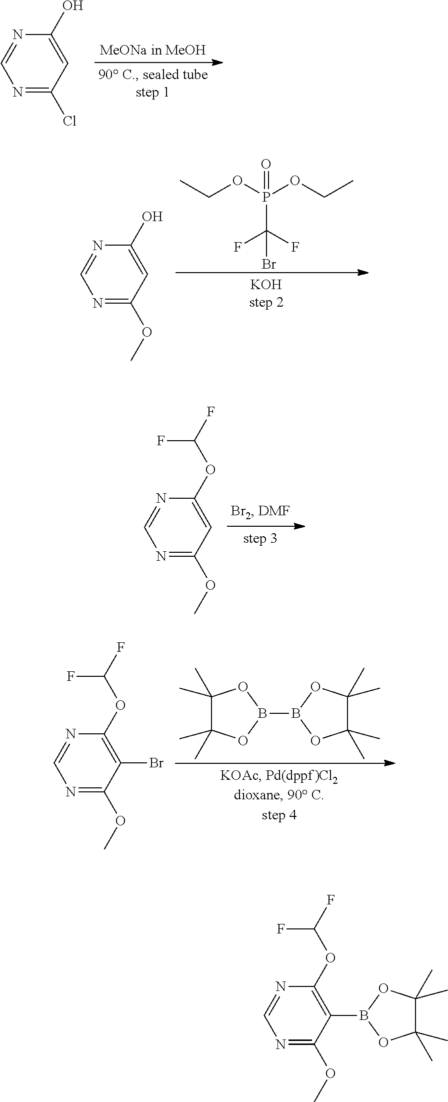

### Step 1. 6-methoxypyrimidin-4-ol

6-chloropyrimidin-4-ol (5g, 38.3 mmol)was dissolved in a solution of sodium methoxide methanol (5 M, 30 mL) and heated at 90° C. in a sealed tube. After cooling to room temperature, solvent was removed and the crude product was purified by column chromatrography on silica gel (eluting with 1/3 EtOAc/PE) to give 6-methoxypyrimidin-4-ol (3.6 g). MS (ESI) m/z 127.0 [M+H]⁺.

### Step 2 to step 4. 4-(difluoromethoxy)-6-methoxy-5-(4,4,5,5-tetramethyl-1,3,2-dioxaborolan-2-yl)pyrimidine

4-(difluoromethoxy)-6-methoxy-5-(4,4,5,5-tetramethyl-1,3,2-dioxaborolan-2-yl)pyrimidine was synthesized from 6-methoxypyrimidin-4-ol following step 2 to step 4 of Intermediate 31. MS (ESI) m/z 303.1 [M+H]⁺.

### Intermediate BB-82. 2-(4-(bromomethyl)phenyl)-1-(propan-2-yl-th)-4-(trifluoromethyl)-1H-imidazole

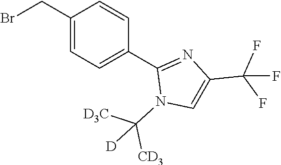 [SMILES: [2H]C([2H])([2H])C([2H])(n1cc(C(F)(F)F)nc1-c1ccc(CBr)cc1)C([2H])([2H])[2H]]

2-(4-(bromomethyl)phenyl)-1-(propan-2-yl-th)-4-(trifluoromethyl)-1H-imidazole was synthesized from methyl 4-(1-isopropyl-4-(trifluoromethyl)-1H-imidazol-2-yl)benzoate ((prepared in Intermediate BB-1)) and 2-iodopropane-1,1,1,2,3,3,3-d7 according to Intermediate BB-3. MS (ESI) m/z 354.1, 356.1 [M+H]⁺.

### Intermediate BB-83. 2-(4-(bromomethyl)phenyl)-1-(ethyl-d₅)-4-(trifluoromethyl)-1H-imidazole

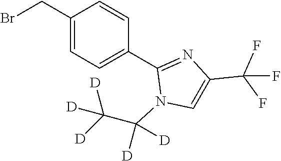 [SMILES: [2H]C([2H])([2H])C([2H])([2H])n1cc(C(F)(F)F)nc1-c1ccc(CBr)cc1]

2-(4-(bromomethyl)phenyl)-1-(ethyl-d₅)-4-(trifluoromethyl)-1H-imidazole was synthesized from methyl 4-(1-isopropyl-4-(trifluoromethyl)-1H-imidazol-2-yl)benzoate (prepared in Intermediate BB-1) and 1-iodoethane-1,1,2,2,2-d5 according to Intermediate BB-1. MS (ESI) m/z 338.0, 340.0 [M+H]⁺.

### Intermediate BB-84. 4-(1-(2-fluoroethyl)-4-(trifluoromethyl)-1H-imidazol-2-yl)aniline

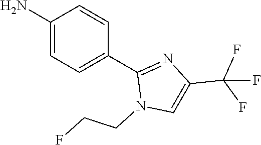 [SMILES: Nc1ccc(-c2nc(C(F)(F)F)cn2CCF)cc1]

4-(1-(2-fluoroethyl)-4-(trifluoromethyl)-1H-imidazol-2-yl)aniline was prepared according to Intermediate BB-14. MS (ESI) m/z 274.1 [M+H]⁺.

### Intermediate BB-85. 4-(1-(ethyl-d₅)-4-(trifluoromethyl)-1H-imidazol-2-yl)aniline

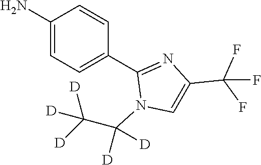 [SMILES: [2H]C([2H])([2H])C([2H])([2H])n1cc(C(F)(F)F)nc1-c1ccc(N)cc1]

4-(1-(ethyl-d₅)-4-(trifluoromethyl)-1H-imidazol-2-yl)aniline was prepared according to

### Intermediate BB-14. MS (ESI) m/z 261.1 [M+H]⁺.

### Intermediate BB-86. 4-(1-(1-fluoropropan-2-yl)-4-(trifluoromethyl)-1H-imidazol-2-yl)phenol

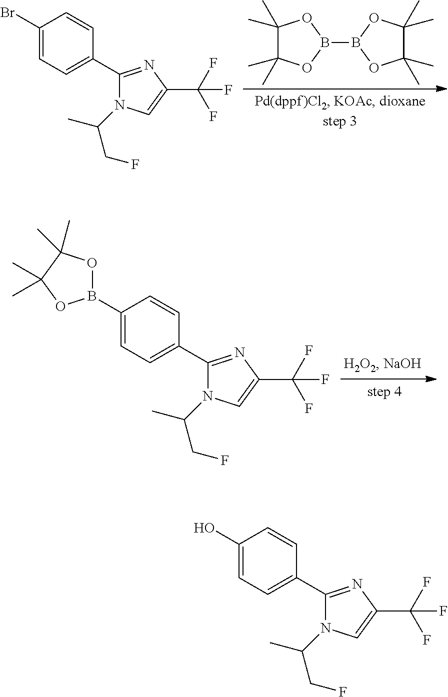

4-(1-(1-fluoropropan-2-yl)-4-(trifluoromethyl)-1H-imidazol-2-yl)phenol was synthesized from 2-(4-bromophenyl)-1-(1-fluoropropan-2-yl)-4-(trifluoromethyl)-1H-imidazole (prepared in Intermediate BB-60) following Intermediate BB-68. m/z 289.1 [M+H]⁺.

Table 29. The compounds listed in Table 29 were synthesized according to Example 364 using the appropriate commercially available reagents and/or intermediates described above.

TABLE 29

| Example | | | |
|---|---|---|---|
| number | Structure | ¹H NMR (δ ppm) | MS (ESI) |
| 372 | 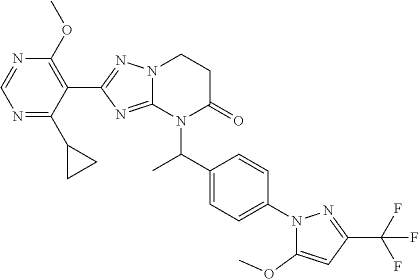 [SMILES: COc1ncnc(C2CC2)c1-c1nc2n(n1)CCC(=O)N2C(C)c1ccc(-n2nc(C(F)(F)F)cc2OC)cc1] | ¹H NMR (400 MHz, Chloroform-d) δ 8.61 (s, 1H), 7.60 (s, 4H), 6.18 (q, J = 7.2 Hz, 1H), 5.93 (s, 1H), 4.45- 4.30 (m, 2H), 3.97 (d, J = 5.9 Hz, 6H), 3.08 (t, J = 7.3 Hz, 2H), 1.98 (d, J = 7.2 Hz, 3H), 1.94 (dt, J = 8.1, 4.5 Hz, 1H), 1.24-1.11 (m, 2H), 0.96-0.84 (m, 2H). | m/z: 555.1 [M + H]⁺ |
| | 2-(4-cyclopropyl-6-methoxypyrimidin-5-yl)-4-(1-(4-(5-methoxy-3- | | |
| | (trifluoromethyl)-1H-pyrazol-1-yl)phenyl)ethyl)-6,7-dihydro- | | |
| | [1,2,4]triazolo[1,5-a]pyrimidin-5(4H)-one | | |
| 373 | 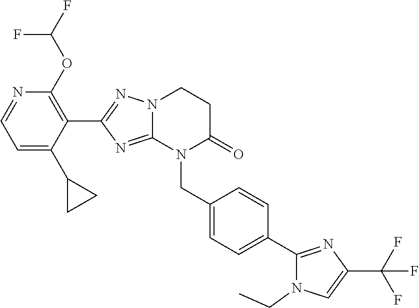 [SMILES: CCn1cc(C(F)(F)F)nc1-c1ccc(CN2C(=O)CCn3nc(-c4c(C5CC5)ccnc4OC(F)F)nc32)cc1] | ¹H NMR (400 MHz, Chloroform-d) δ 8.63 (s, 1H), 7.63 (s, 2H), 7.52 (s, 2H), 7.48 (t, J = 72.0 Hz, 1H), 7.36 (s, 1H), 6.36 (s, 1H), 5.21 (s, 2H), 4.38 (s, 2H), 4.06 (s, 2H), 3.12 (s, 2H), 2.93 (s, 1H), 1.44 (s, 3H), 1.12 (d, J = 7.2 Hz, 2H), 0.86-0.79 (m, 2H). | m/z: 574.2 [M + H]⁺ |
| | 2-(4-cyclopropyl-2-(difluoromethoxy)pyridin-3-yl)-4-(4-(1-ethyl-4- | | |
| | (trifluoromethyl)-1H-imidazol-2-yl)benzyl)-6,7-dihydro- | | |
| | [1,2,4]triazolo[1,5-a]pyrimidin-5(4H)-one | | |
| 374 | 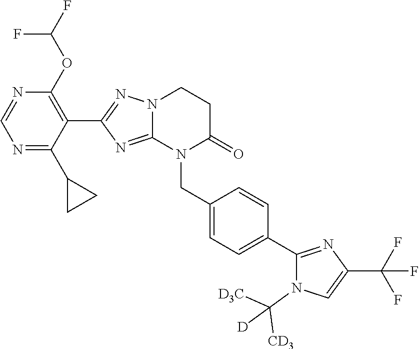 [SMILES: [2H]C([2H])([2H])C([2H])(n1cc(C(F)(F)F)nc1-c1ccc(CN2C(=O)CCn3nc(-c4c(OC(F)F)ncnc4C4CC4)nc32)cc1)C([2H])([2H])[2H]] | ¹H NMR (400 MHz, Chloroform-d) δ 8.64 (s, 1H), 7.67 (d, J = 7.3 Hz, 2H), 7.54 (t, J = 72.0 Hz, 1H), 7.48 (d, J = 7.3 Hz, 2H), 7.39 (s, 1H), 5.20 (s, 2H), 4.41 (s, 2H), 3.13 (s, 2H), 2.24 (s, 1H), 1.28 (s, 2H), 1.08-1.00 (m, 2H). | m/z: 596.1 [M + H]⁺ |
| | 2-(4-cyclopropyl-6-(difluoromethoxy)pyrimidin-5-yl)-4-(4-(1-(propan- | | |
| | 2-yl-d₇)-4-(trifluoromethyl)-1H-imidazol-2-yl)benzyl)-6,7-dihydro- | | |
| | [1,2,4]triazolo[1,5-a]pyrimidin-5(4H)-one | | |
| 375 | 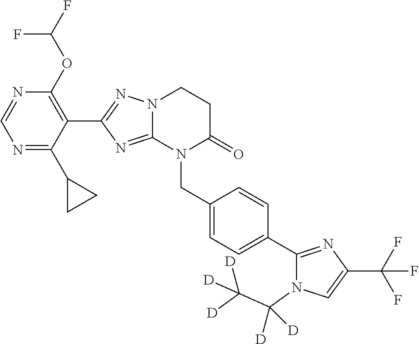 [SMILES: [2H]C([2H])([2H])C([2H])([2H])n1cc(C(F)(F)F)nc1-c1ccc(CN2C(=O)CCn3nc(-c4c(OC(F)F)ncnc4C4CC4)nc32)cc1] | ¹H NMR (400 MHz, Methanol-d₄) δ 8.66 (d, J = 1.5 Hz, 1H), 7.78 (s, 1H), 7.66 (t, J = 72.0 Hz, 1H), 7.65 (dt, J = 5.9, 1.7 Hz, 2H), 7.54 (dd, J = 8.3, 1.7 Hz, 2H), 5.22 (s, 2H), 4.50-4.43 (m, 2H), 3.23- 3.16 (m, 2H), 2.20 (dq, J = 8.1, 4.2, 3.7 Hz, 1H), 1.21 (dq, J = 5.9, 3.9 Hz, 2H), 1.01 (dq, J = 7.1, 3.6 Hz, 2H). | m/z: 580.2 [M + H]⁺ |
| | 2-(4-cyclopropyl-6-(difluoromethoxy)pyrimidin-5-yl)-4-(4-(1-(ethyl- | | |
| | d₅)-4-(trifluoromethyl)-1H-imidazol-2-yl)benzyl)-6,7-dihydro- | | |
| | [1,2,4]triazolo[1,5-a]pyrimidin-5(4H)-one | | |
| 376 | 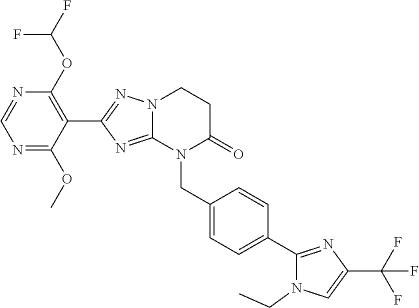 [SMILES: CCn1cc(C(F)(F)F)nc1-c1ccc(CN2C(=O)CCn3nc(-c4c(OC)ncnc4OC(F)F)nc32)cc1] | ¹H NMR (400 MHz, Chloroform-d) δ 8.51 (s, 1H), 7.68 (d, J = 7.9 Hz, 2H), 7.55 (t, J = 72.0 Hz, 1H), 7.53 (d, J = 7.9 Hz, 2H), 7.35 (s, 1H), 5.18 (s, 2H), 4.39 (t, J = 7.3 Hz, 2H), 4.04 (d, J = 5.6 Hz, 5H), 3.11 (t, J = 7.3 Hz, 2H), 1.42 (t, J = 7.2 Hz, 3H). | m/z: 565.2 [M + H]⁺ |
| | 2-(4-(difluoromethoxy)-6-methoxypyrimidin-5-yl)-4-(4-(1-ethyl-4- | | |
| | (trifluoromethyl)-1H-imidazol-2-yl)benzyl)-6,7-dihydro- | | |
| | [1,2,4]triazolo[1,5-a]pyrimidin-5(4H)-one | | |
| 377 | 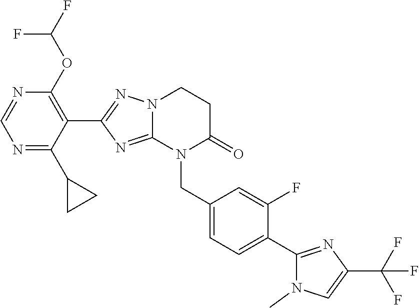 [SMILES: Cn1cc(C(F)(F)F)nc1-c1ccc(CN2C(=O)CCn3nc(-c4c(OC(F)F)ncnc4C4CC4)nc32)cc1F] | ¹H NMR (400 MHz, Chloroform-d) δ 8.65 (s, 1H), 7.55 (t, J = 7.6 Hz, 1H), 7.52 (t, J = 72.0 Hz, 1H), 7.47 (d, J = 7.9 Hz, 1H), 7.40 (d, J = 10.7 Hz, 1H), 7.34 (s, 1H), 5.19 (s, 2H), 4.43 (t, J = 7.4 Hz, 2H), 3.62 (d, J = 2.3 Hz, 3H), 3.15 (t, J = 7.4 Hz, 2H), 2.20 (tt, J = 8.4, 4.5 Hz, 1H), 1.31-1.27 (m, 2H), 1.07 (dt, J = 7.8, 3.5 Hz, 2H). | m/z: 579.2 [M + H]⁺ |
| | 2-(4-cyclopropyl-6-(difluoromethoxy)pyrimidin-5-yl)-4-(3-fluoro-4-(1- | | |
| | methyl-4-(trifluoromethyl)-1H-imidazol-2-yl)benzyl)-6,7-dihydro- | | |
| | [1,2,4]triazolo[1,5-a]pyrimidin-5(4H)-one | | |
| 378 | 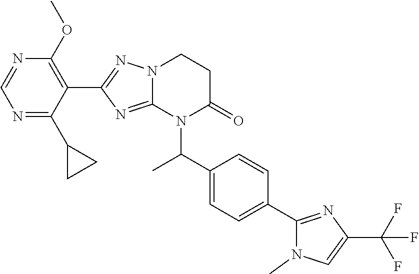 [SMILES: COc1ncnc(C2CC2)c1-c1nc2n(n1)CCC(=O)N2C(C)c1ccc(-c2nc(C(F)(F)F)cn2C)cc1] | ¹H NMR (400 MHz, Chloroform-d) δ 8.59 (s, 1H), 7.62 (d, J = 6.8 Hz, 2H), 7.55 (d, J = 6.9 Hz, 2H), 7.28 (s, 1H), 6.15 (q, J = 6.5 Hz, 1H), 4.38 (s, 2H), 3.95 (s, 3H), 3.74 (s, 3H), 3.07 (s, 2H), 1.98 (d, J = 6.9 Hz, 4H), 1.18 (d, J = 8.6 Hz, 2H), 0.90 (d, J = 6.9 Hz, 2H). | m/z: 539.2 [M + H]⁺ |
| | 2-(4-cyclopropyl-6-methoxypyrimidin-5-yl)-4-(1-(4-(1-methyl-4- | | |
| | (trifluoromethyl)-1H-imidazol-2-yl)phenyl)ethyl)-6,7-dihydro- | | |
| | [1,2,4]triazolo[1,5-a]pyrimidin-5(4H)-one | | |
| 379 | 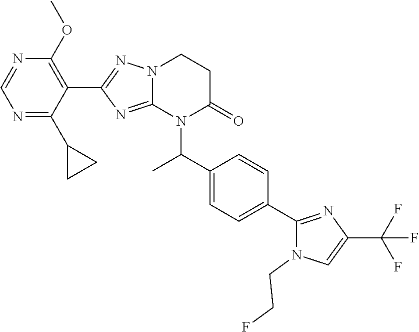 [SMILES: COc1ncnc(C2CC2)c1-c1nc2n(n1)CCC(=O)N2C(C)c1ccc(-c2nc(C(F)(F)F)cn2CCF)cc1] | ¹H NMR (400 MHz, Methanol-d₄) δ 8.55 (s, 1H), 7.80 (s, 1H), 7.64 (d, J = 8.2 Hz, 2H), 7.55 (d, J = 8.4 Hz, 2H), 6.14 (q, J = 7.2 Hz, 1H), 4.74 (t, J = 4.7 Hz, 1H), 4.62 (t, J = 4.7 Hz, 1H), 4.42 (dd, J = 8.9, 5.8 Hz, 3H), 4.34 (t, J = 4.7 Hz, 1H), 3.91 (s, 3H), 3.18-3.10 (m, 2H), 2.00-1.94 (m, 4H), 1.13-1.07 (m, 2H), 0.92-0.87 (m, 2H). | m/z: 571.2 [M + H]⁺ |
| | 2-(4-cyclopropyl-6-methoxypyrimidin-5-yl)-4-(1-(4-(1-(2-fluoroethyl)- | | |
| | 4-(trifluoromethyl)-1H-imidazol-2-yl)phenyl)ethyl)-6,7-dihydro- | | |
| | [1,2,4]triazolo[1,5-a]pyrimidin-5(4H)-one | | |
| 380 | 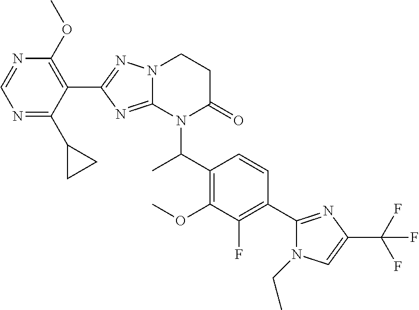 [SMILES: CCn1cc(C(F)(F)F)nc1-c1ccc(C(C)N2C(=O)CCn3nc(-c4c(OC)ncnc4C4CC4)nc32)c(OC)c1F] | ¹H NMR (400 MHz, Methanol-d₄) δ 8.58 (s, 1H), 7.84 (q, J = 1.2 Hz, 1H), 7.64- 7.57 (m, 1H), 7.19 (dd, J = 8.2, 6.6 Hz, 1H), 6.17 (q, J = 7.1 Hz, 1H), 4.42-4.33 (m, 2H), 3.96 (d, J = 7.3 Hz, 2H), 3.94 (s, 3H), 3.88 (d, J = 2.1 Hz, 3H), 3.13 (ddd, J = 16.1, 8.4, 7.5 Hz, 1H), 3.01 (dt, J = 16.8, 6.6 Hz, 1H), 2.08-2.00 (m, 1H), 1.91 (d, J = 7.2 Hz, 3H), 1.35 (t, J = 7.3 Hz, 3H), 1.18-1.10 (m, 2H), 0.96 (dd, J = 8.2, 2.4 Hz, 2H). | m/z: 601.2 [M + H]⁺ |
| | 2-(4-cyclopropyl-6-methoxypyrimidin-5-yl)-4-(1-(4-(1-ethyl-4- | | |
| | (trifluoromethyl)-1H-imidazol-2-yl)-3-fluoro-2-methoxyphenyl)ethyl)- | | |
| | 6,7-dihydro-[1,2,4]triazolo[1,5-a]pyrimidin-5(4H)-one | | |

Table 30. The compounds listed in Table 30 were synthesized according to Example 347 using the appropriate commercially available reagents and/or intermediates described above. Enantiomers, when generated, were separated by chiral prep-HPLC and absolute stereochemistries were arbitrarily assigned.

TABLE 30

| Example | | | |
|---|---|---|---|
| number | Structure | ¹H NMR (δ ppm) | MS (ESI) |
| 381 | 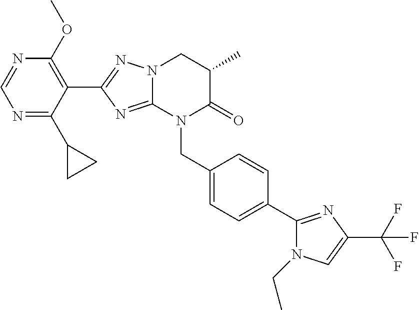 [SMILES: CCn1cc(C(F)(F)F)nc1-c1ccc(CN2C(=O)[C@@H](C)Cn3nc(-c4c(OC)ncnc4C4CC4)nc32)cc1] | ¹H NMR (400 MHz, Chloroform-d) δ 8.62 (s, 1H), 7.62 (d, J = 8.1 Hz, 2H) 7.49 (d, J = 8.1 Hz, 2H), 7.35 (s, 1H), 5.25-5.14 (m, 2H), 4.50 (dd, J = 12.9, 7.2 Hz, 1H), 4.08-3.97 (m, 3H), 3.96 (s, 3H), 3.13 (dt, J = 11.0, 7.0 Hz, 1H), 2.01 (tt, J = 8.3, 4.7 Hz, 1H), 1.45-1.38 (m, 6H), 1.23 (td, J = 4.8, 1.8 Hz, 2H), 0.95 (dd, J = 8.0, 2.9 Hz, 2H). | m/z: 553.3 [M + H]⁺ |
| | (S)-2-(4-cyclopropyl-6-methoxypyrimidin-5-yl)-4-(4-(1-ethyl-4- | | |
| | (trifluoromethyl)-1H-imidazol-2-yl)benzyl)-6-methyl-6,7-dihydro- | | |
| | [1,2,4]triazolo[1,5-a]pyrimidin-5(4H)-one | | |
| 382 | 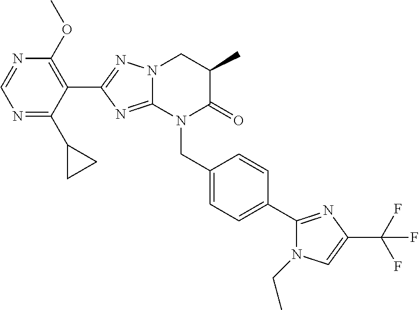 [SMILES: CCn1cc(C(F)(F)F)nc1-c1ccc(CN2C(=O)[C@H](C)Cn3nc(-c4c(OC)ncnc4C4CC4)nc32)cc1] | ¹H NMR (400 MHz, Chloroform-d) δ 8.62 (s, 1H), 7.62 (d, J = 8.1 Hz, 2H), 7.49 (d, J = 8.1 Hz, 2H), 7.35 (s, 1H), 5.25-5.15 (m, 2H), 4.50 (dd, J = 12.9, 7.2 Hz, 1H), 4.08-3.97 (m, 3H), 3.96 (s, 3H), 3.13 (dt, J = 11.0, 7.0 Hz, 1H), 2.01 (dt, J = 8.0, 3.6 Hz, 1H), 1.44-1.39 (m, 6H), 1.23 (td, J = 4.8, 1.8 Hz, 2H), 0.95 (dd, J = 8.0, 2.9 Hz, 2H). | m/z: 553.3 [M + H]⁺ |
| | (R)-2-(4-cyclopropyl-6-methoxypyrimidin-5-yl)-4-(4-(1-ethyl-4- | | |
| | (trifluoromethyl)-1H-imidazol-2-yl)benzyl)-6-methyl-6,7-dihydro- | | |
| | [1,2,4]triazolo[1,5-a]pyrimidin-5(4H)-one | | |
| 383 | 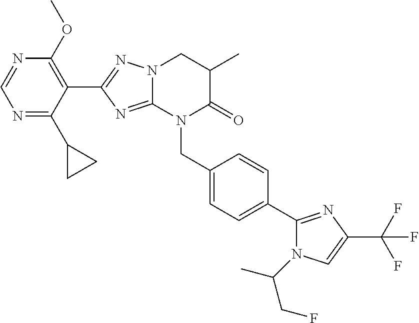 [SMILES: COc1ncnc(C2CC2)c1-c1nc2n(n1)CC(C)C(=O)N2Cc1ccc(-c2nc(C(F)(F)F)cn2C(C)CF)cc1] | ¹H NMR (400 MHz, Methanol-d₄) δ 8.58 (d, J = 2.6 Hz, 1H), 7.95 (s, 1H), 7.63-7.59 (m, 2H), 7.52 (d, J = 8.3 Hz, 2H), 5.21 (s, 2H), 4.71-4.62 (m, 2H), 4.60-4.52 (m, 2H), 4.10 (dd, J = 12.8, 11.0 Hz, 1H), 3.93 (d, J = 2.1 Hz, 3H), 2.05 (dt, J = 8.2, 3.9 Hz, 1H), 1.45 (dt, J = 6.9, 1.2 Hz, 3H), 1.39 (dd, J = 7.3, 2.3 Hz, 3H), 1.14 (dd, J = 4.6, 2.7 Hz, 2H), 0.97-0.90 (m, 3H). | m/z: 585.3 [M + H]⁺ |
| | 2-(4-cyclopropyl-6-methoxypyrimidin-5-yl)-4-(4-(1-(1-fluoropropan- | | |
| | 2-yl)-4-(trifluoromethyl)-1H-imidazol-2-yl)benzyl)-6-methyl-6,7- | | |
| | dihydro-[1,2,4]triazolo[1,5-a]pyrimidin-5(4H)-one | | |
| 384 | 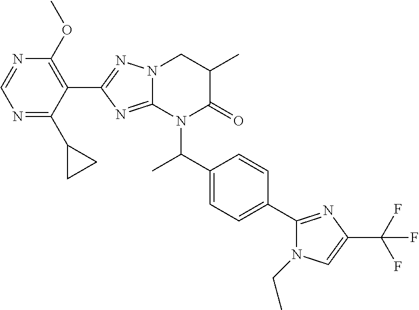 [SMILES: CCn1cc(C(F)(F)F)nc1-c1ccc(C(C)N2C(=O)C(C)Cn3nc(-c4c(OC)ncnc4C4CC4)nc32)cc1] | ¹H NMR (400 MHz, Chloroform-d) δ 8.59 (d, J = 4.3 Hz, 1H), 7.61 (dd, J = 11.1, 7.9 Hz, 2H), 7.49 (d, J = 7.9 Hz, 2H), 7.34 (s, 1H), 6.14 (q, J = 7.9 Hz, 1H), 4.47 (dt, J = 11.8, 5.4 Hz, 1H), 4.08- 4.02 (m, 2H), 3.99 (d, J = 11.6 Hz, 1H), 3.94 (d, J = 3.7 Hz, 3H), 3.08 (dt, J = 11.3, 6.6 Hz, 1H), 1.97 (dd, J = 14.6, 7.3 Hz, 4H), 1.39 (dt, J = 22.2, 7.5 Hz, 6H), 1.17 (d, J = 13.5 Hz, 2H), 0.95-0.85 (m, 2H). | m/z: 567.3 [M + H]⁺ |
| | 2-(4-cyclopropyl-6-methoxypyrimidin-5-yl)-4-(1-(4-(1-ethyl-4- | | |
| | (trifluoromethyl)-1H-imidazol-2-yl)phenyl)ethyl)-6-methyl-6,7- | | |
| | dihydro-[1,2,4]triazolo[1,5-a]pyrimidin-5(4H)-one | | |

Table 31. The compounds listed in Table 31 were synthesized according to Example 337 using the appropriate commercially available reagents and/or intermediates described above.

TABLE 31

| Example | | | |
|---|---|---|---|
| number | Structure | ¹H NMR (δ ppm) | MS (ESI) |
| 385 | 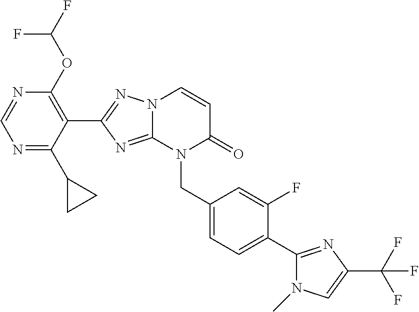 [SMILES: Cn1cc(C(F)(F)F)nc1-c1ccc(Cn2c(=O)ccn3nc(-c4c(OC(F)F)ncnc4C4CC4)nc23)cc1F] | ¹H NMR (400 MHz, Chloroform-d) δ 8.69 (s, 1H), 8.21 (d, J = 7.9 Hz, 1H), 7.56 (d, J = 4.0 Hz, 2H), 7.54 (t, J = 72.0 Hz, 1H), 7.49 (d, J = 10.8 Hz, 1H), 7.33 (s, 1H), 6.36 (d, J = 7.9 Hz, 1H), 5.44 (s, 2H), 3.61 (d, J = 2.2 Hz, 3H), 2.21 (tt, J = 8.1, 4.5 Hz, 1H), 1.32 (dd, J = 4.4, 3.0 Hz, 2H), 1.08 (dq, J = 7.1, 3.8 Hz, 2H). | m/z: 577.1 [M + H]⁺ |
| | 2-(4-cyclopropyl-6-(difluoromethoxy)pyrimidin-5-yl)-4-(3-fluoro-4- | | |
| | (1-methyl-4-(trifluoromethyl)-1H-imidazol-2-yl)benzyl)- | | |
| | [1,2,4]triazolo[1,5-a]pyrimidin-5(4H)-one | | |
| 386 | 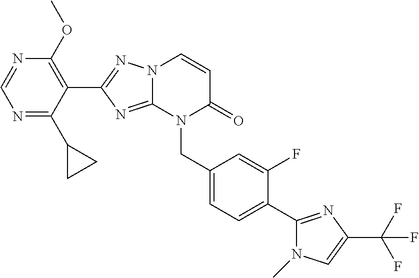 [SMILES: COc1ncnc(C2CC2)c1-c1nc2n(Cc3ccc(-c4nc(C(F)(F)F)cn4C)c(F)c3)c(=O)ccn2n1] | ¹H NMR (400 MHz, Chloroform-d) δ 8.66 (s, 1H), 8.20 (d, J = 7.2 Hz, 1H), 7.61- 7.48 (m, 3H), 7.34 (s, 1H), 6.33 (d, J = 12 Hz, 1H), 5.45 (s, 2H), 3.98 (s, 3H), 3.62 (s, 3H), 2.03 (d, J = 10.9 Hz, 1H), 1.03-0.92 (m, 2H). | m/z: 541.1 [M + H]⁺ |
| | 2-(4-cyclopropyl-6-methoxypyrimidin-5-yl)-4-(3-fluoro-4-(1-methyl- | | |
| | 4-(trifluoromethyl)-1H-imidazol-2-yl)benzyl)- | | |
| | [1,2,4]triazolo[1,5-a]pyrimidin-5(4H)-one | | |

Table 32. The compounds listed in Table 32 were synthesized according to Example 237 using the appropriate commercially available reagents and/or intermediates described above.

TABLE 32

| Example | | | |
|---|---|---|---|
| number | Structure | ¹H NMR (δ ppm) | MS (ESI) |
| 387 | 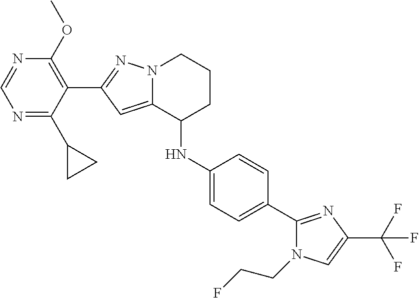 [SMILES: COc1ncnc(C2CC2)c1-c1cc2n(n1)CCCC2Nc1ccc(-c2nc(C(F)(F)F)cn2CCF)cc1] | ¹H NMR (400 MHz, Methanol-d₄) δ 8.53-8.44 (m, 1H), 7.72 (d, J = 2.2 Hz, 1H), 7.37 (dq, J = 8.9, 2.5 Hz, 2H), 6.88 (dq, J =9.0, 2.5 Hz, 2H), 6.28 (d, J = 3.7 Hz, 1H), 4.94 (dd, J = 8.5, 4.4 Hz, 1H), 4.75 (q, J = 4.3, 3.8 Hz, 1H), 4.63 (q, J = 4.6 Hz, 1H), 4.37 (dt, J = 26.5, 4.7 Hz, 2H), 4.29-4.13 (m, 2H), 3.91-3.86 (m, 3H), 2.31 (dd, J = 11.4, 4.9 Hz, 2H), 2.22-2.07 (m, 2H), 1.96- 1.83 (m, 1H), 1.10 (dt, J = 6.5, 3.0 Hz, 2H), 0.91 (dt, J = 9.2, 3.3 Hz, 2H). | m/z: 542.2 [M + H]⁺ |
| | 2-(4-cyclopropyl-6-methoxypyrimidin-5-yl)-N-(4-(1-(2-fluoroethyl)-4- | | |
| | (trifluoromethyl)-1H-imidazol-2-yl)phenyl)-4,5,6,7- | | |
| | tetrahydropyrazolo[1,5-a]pyridin-4-amine | | |
| 388 | 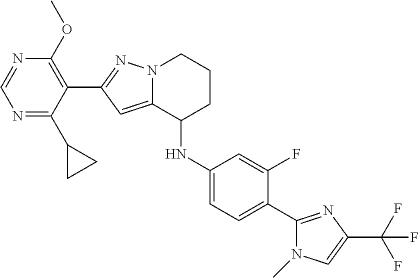 [SMILES: COc1ncnc(C2CC2)c1-c1cc2n(n1)CCCC2Nc1ccc(-c2nc(C(F)(F)F)cn2C)c(F)c1] | ¹H NMR (400 MHz, Methanol-d₄) δ 8.49 (s, 1H), 7.70-7.63 (m, 1H), 7.27 (t, J = 8.5 Hz, 1H), 6.69 (dd, J = 8.5, 2.2 Hz, 1H), 6.63 (dd, J = 13.3, 2.2 Hz, 1H), 6.30 (s, 1H), 4.93 (dd, J = 8.1, 4.4 Hz, 1H), 4.30-4.13 (m, 2H), 3.89 (s, 3H), 3 .64 (d, J = 1.6 Hz, 3H), 2.33-2.27 (m, 2H), 2.21- 2.10 (m, 2H), 1.90 (d, J = 8.4 Hz, 1H), 1.12-1.08 (m, 2H), 0.94-0.89 (m, 2H). | m/z: 528.2 [M + H]⁺ |
| | 2-(4-cyclopropyl-6-methoxypyrimidin-5-yl)-N-(3-fluoro-4-(1-methyl- | | |
| | 4-(trifluoromethyl)-1H-imidazol-2-yl)phenyl)-4,5,6,7- | | |
| | tetrahydropyrazolo[1,5-a]pyridin-4-amine | | |
| 389 | 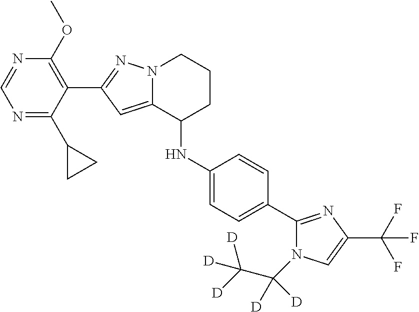 [SMILES: [2H]C([2H])([2H])C([2H])([2H])n1cc(C(F)(F)F)nc1-c1ccc(NC2CCCn3nc(-c4c(OC)ncnc4C4CC4)cc32)cc1] | ¹H NMR (400 MHz, Methanol-d₄) δ 8.49 (s, 1H), 7.74-7.61 (m, 1H), 7.37 (d, J = 8.4 Hz, 2H), 6.88 (d, J = 8.5 Hz, 2H), 6.28 (s, 1H), 4.94 (dd, J = 8.3, 4.5 Hz, 1H), 4.32-4.12 (m, 2H), 3.92-3.84 (m, 3H), 2.41-2.26 (m, 2H), 2.24-2.09 (m, 2H), 1.97-1.83 (m, 1H), 1.09 (dt, J = 5.9, 3.0 Hz, 2H), 0.91 (dd, J = 8.0, 3.6 Hz, 2H). | m/z: 529.2 [M + H]⁺ |
| | 2-(4-cyclopropyl-6-methoxypyrimidin-5-yl)-N-(4-(1-(ethyl-d₅)-4- | | |
| | (trifluoromethyl)-1H-imidazol-2-yl)phenyl)-4,5,6,7- | | |
| | tetrahydropyrazolo[1,5-a]pyridin-4-amine | | |
| 390 | 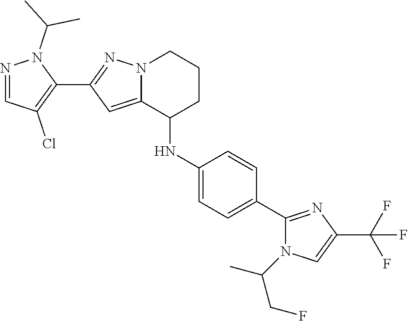 [SMILES: CC(CF)n1cc(C(F)(F)F)nc1-c1ccc(NC2CCCn3nc(-c4c(Cl)cnn4C(C)C)cc32)cc1] | ¹H NMR (400 MHz, Methanol-d₄) δ 8.59 (s, 1H), 7.59-7.49 (m, 3H), 7.02 (d, J = 8.3 Hz, 2H), 6.53 (s, 1H), 5.16-4.96 (m, 3H), 4.81 (s, 1H), 4.69 (s, 1H), 4.38-4.17 (m, 2H), 2.27 (d, J = 47.4 Hz, 3H), 1.94 (d, J = 7.8 Hz, 1H), 1.59 (d, J = 6.5 Hz, 3H), 1.41 (d, J = 6.6 Hz, 6H). | m/z: 550.3 [M + H]⁺ |
| | 2-(4-chloro-1-isopropyl-1H-pyrazol-5-yl)-N-(4-(1-(1-fluoropropan-2- | | |
| | yl)-4-(trifluoromethyl)-1H-imidazol-2-yl)phenyl)-4,5,6,7- | | |
| | tetrahydropyrazolo[1,5-a]pyridin-4-amine | | |

### Example 391. 2-(4-cyclopropyl-6-methoxypyrimidin-5-yl)-4-(4-(1-ethyl-4-(trifluoromethyl)-1H-imidazol-2-yl)benzyl)-6-methyl-[1,2,4]triazolo[1,5-α]pyrimidin-5(4H)-one

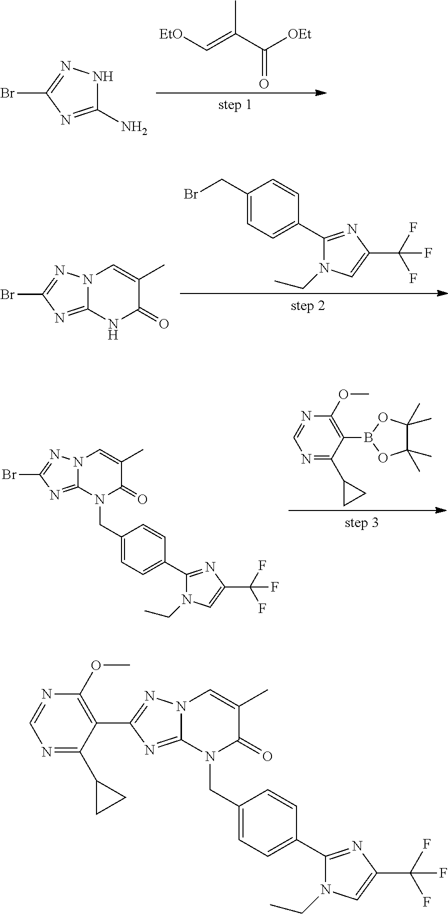

### Step 1 to step 3. 2-(4-cyclopropyl-6-methoxypyrimidin-5-yl)-4-(4-(1-ethyl-4-(trifluoromethyl)-1H-imidazol-2-yl)benzyl)-6-methyl-[1,2,4]triazolo[1,5-α]pyrimidin-5(4H)-one

2-(4-cyclopropyl-6-methoxypyrimidin-5-yl)-4-(4-(1-ethyl-4-(trifluoromethyl)-1H-imidazol-2-yl)benzyl)-6-methyl-[1,2,4]triazolo[1,5-α]pyrimidin-5(4H)-one was prepared from 3-bromo-1H-1,2,4-triazol-5-amine and ethyl (E)-3-ethoxy-2-methylacrylate following step 3 to step 5 of Example 337.

Table 33. The compounds listed in Table 33 were synthesized according to Example 392 using the appropriate commercially available reagents and/or intermediates described above.

TABLE 33

| Example | | | |
|---|---|---|---|
| number | Structure | ¹H NMR (δ ppm) | MS (ESI) |
| 391 | 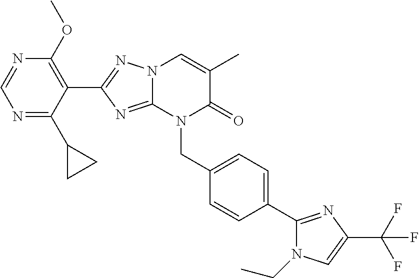 [SMILES: CCn1cc(C(F)(F)F)nc1-c1ccc(Cn2c(=O)c(C)cn3nc(-c4c(OC)ncnc4C4CC4)nc23)cc1] | ¹H NMR (400 MHz, Chloroform-d) δ 8.65 (s, 1H), 8.10 (d, J = 1.5 Hz, 1H), 7.74 (d, J = 8.0 Hz, 2H), 7.52 (d, J = 8.1 Hz, 2H), 7.35 (d, J = 1.3 Hz, 1H), 5.48 (s, 2H), 4.03 (q, J = 7.3 Hz, 2H), 3.97 (s, 3H), 2.18 (d, J = 1.3 Hz, 3H), 2.01 (tt, J = 8.3, 4.6 Hz, 1H), 1.42 (t, J = 7.3 Hz, 3H), 1.25 (t, J = 2.3 Hz, 2H), 0.96 (dt, J = 8.2, 3.4 Hz, 2H). | m/z: 551.2 [M + H]⁺ |
| | 2-(4-cyclopropyl-6-methoxypyrimidin-5-yl)-4-(4-(1-ethyl-4- | | |
| | (trifluoromethyl)-1H-imidazol-2-yl)benzyl)-6-methyl- | | |
| | [1,2,4]triazolo[1,5-a]pyrimidin-5(4H)-one | | |
| 392 | 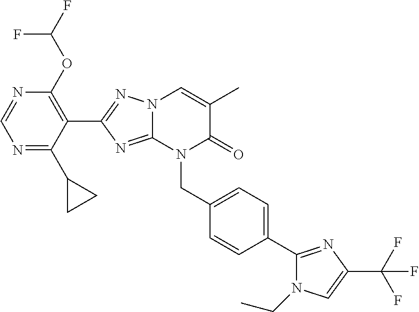 [SMILES: CCn1cc(C(F)(F)F)nc1-c1ccc(Cn2c(=O)c(C)cn3nc(-c4c(OC(F)F)ncnc4C4CC4)nc23)cc1] | ¹H NMR (400 MHz, Chloroform-d) δ 8.68 (s, 1H), 8.10 (d, J = 1.5 Hz, 1H), 7.78 (d, J = 8.1 Hz, 2H), 7.56 (t, J = 72.0 Hz, 1H), 7.54 (J = 8.1 Hz, 2H), 7.35 (d, J = 1.3 Hz, 1H), 5.48 (s, 2H), 4.03 (q, J = 13 Hz, 2H), 2.23 (dt, J = 8.1, 3.6 Hz, 1H), 2.19 (d, J = 1.5 Hz, 3H), 1.42 (t, J = 7.3 Hz, 3H), 1.33-1.30 (m, 2H), 1.06 (dq, J = 7.2, 3.9 Hz, 2H). | m/z: 587.2 [M + H]⁺ |
| | 2-(4-cyclopropyl-6-(difluoromethoxy)pyrimidin-5-yl)-4-(4-(1-ethyl-4- | | |
| | (trifluoromethyl)-1H-imidazol-2-yl)benzyl)-6-methyl- | | |
| | [1,2,4]triazolo[1,5-a]pyrimidin-5(4H)-one | | |

### Example 393. (4S)-2-(4-cyclopropyl-6-methoxypyrimidin-5-yl)-4-(4-(1-(1-fluoropropan-2-yl)-4-(trifluoromethyl)-1H-imidazol-2-yl)phenoxy)-4,5,6,7-tetrahydropyrazolo[1,5-α]pyridine

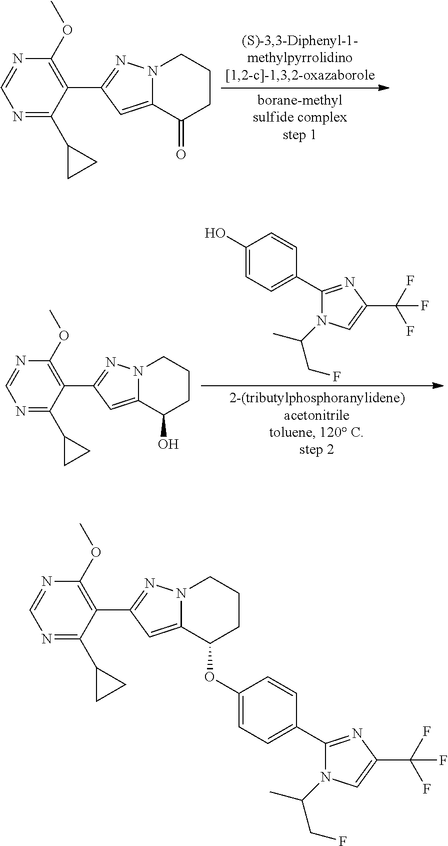 [SMILES: COc1ncnc(C2CC2)c1-c1cc2n(n1)CCCC2=O ; COc1ncnc(C2CC2)c1-c1cc2n(n1)CCC[C@H]2O ; CC(CF)n1cc(C(F)(F)F)nc1-c1ccc(O)cc1 ; COc1ncnc(C2CC2)c1-c1cc2n(n1)CCC[C@@H]2Oc1ccc(-c2nc(C(F)(F)F)cn2C(C)CF)cc1]

### Step 1. (R)-2-(4-cyclopropyl-6-methoxypyrimidin-5-yl)-4,5,6,7-tetrahydropyrazolo[1,5-α]pyridin-4-ol

To a solution of borane-methyl sulfide complex (2 M in THF, 5.6 mL, 11.2 mmol) in dry THF (19 mL) at −15° C. was added (S)-3,3-Diphenyl-1-methylpyrrolidino[1,2-c]-1,3,2-oxazaborole (1 M in toluene, 5.6 mL, 5.6 mmol). The solution was stirred for 30 min then a solution of 2-(4-cyclopropyl-6-methoxypyrimidin-5-yl)-6,7-dihydropyrazolo[1,5-α]pyridin-4(5H)-one (800 mg, 3.72 mmol) in dry THF (1 mL) was added. The resulting mixture was stirred at −15° C. for 1 h then warmed to room temperature, quenched with water (50 mL) and extracted with EtOAc (50 mL×3). The combined organic layers were dried over anhydrous Na₂SO₄, concentrated under vacuum and purified by column chromatrography on silica gel (eluting with 1/4 EtOAc/PE) to afford (R)-2-(4-cyclopropyl-6-methoxypyrimidin-5-yl)-4,5,6,7-tetrahydropyrazolo[1,5-α]pyridin-4-ol (580 mg). MS (ESI) m/z 287.1 [M+H]⁺.

### Step 2. (4S)-2-(4-cyclopropyl-6-methoxypyrimidin-5-yl)-4-(4-(1-(1-fluoropropan-2-yl)-4-(trifluoromethyl)-1H-imidazol-2-yl)phenoxy)-4,5,6,7-tetrahydropyrazolo[1,5-α]pyridine

To a solution of (4S)-2-(4-cyclopropyl-6-methoxypyrimidin-5-yl)-4-(4-(1-(1-fluoropropan-2-yl)-4-(trifluoromethyl)-1H-imidazol-2-yl)phenoxy)-4,5,6,7-tetrahydropyrazolo[1,5-α]pyridine (153 mg, 0.53 mmol) in dry tolunene (3 mL) was added 4-(1-(1-fluoropropan-2-yl)-4-(trifluoromethyl)-1H-imidazol-2-yl)phenol (154 mg, 0.53 mmol) and 2-(tributylphosphoranylidene)acetonitrile (192 mg, 0.8 mmol). The resulting mixture was heated at 120° C. for 6 h in a sealed tube. The solvent was removed and the residue was purified by prep-HPLC (condition 3) to afford Example 393 (4S)-2-(4-cyclopropyl-6-methoxypyrimidin-5-yl)-4-(4-(1-(1-fluoropropan-2-yl)-4-(trifluoromethyl)-1H-imidazol-2-yl)phenoxy)-4,5,6,7-tetrahydropyrazolo[1,5-α]pyridine (20.5 mg). ¹H NMR (400 MHz, Methanol-d₄) δ 8.50 (d, J=1.8 Hz, 1H), 7.93 (d, J=1.5 Hz, 1H), 7.55-7.51 (m, 2H), 7.26-7.22 (m, 2H), 6.37 (d, J=1.8 Hz, 1H), 5.80 (t, J=4.7 Hz, 1H), 4.75-4.64 (m, 2H), 4.55 (dq, J=4.7, 1.4 Hz, 1H), 4.34 (dt, J=12.6, 5.3 Hz, 1H), 4.19 (m, 1H), 3.90 (d, J=1.7 Hz, 3H), 2.45 (td, J=8.9, 4.9 Hz, 1H), 2.31-2.24 (m, 2H), 2.19-2.07 (m, 2H), 1.46 (dt, J=7.0, 1.7 Hz, 3H), 1.10 (dq, J=4.6, 1.5 Hz, 2H), 0.91 (dt, J=6.8, 2.4 Hz, 2H). MS (ESI) m/z 557.2 [M+H]⁺.

### Example 394

Deubiquitination Assay for USP1/UAF1 and USP1 ES2 Activity and Inhibitor Testing

USPI/UAF1 Assay

Certain Compounds of the Disclosure were assessed for USP1/UAF1 activity in a Ubiquitin Rhodamine assay modified from those described previously.

Deubiquitinase activity was measured using ubiquitin-rhodamine 110 as a substrate. Cleavage of the amide bond between rhodamine and the c-terminal glycine of ubiquitin yields an increase in fluorescence signal. The assay was conducted in 20 μl total volume of assay buffer (50 mM Tris-HCl, pH 7.8, 0.5 mM EDTA, 0.01% Bovine Serum Albumin, 1 mM DTT, 0.01% Tween-20), and 0.05 nM USP1/UAF1 enzyme. Reaction was initiated by addition of 150 nM Ubiquitin-rhodamine (Boston Biochem) substrate.

Compounds of the Disclosure, dissolved in DMSO were tested in dose response format, beginning at 10 04.

The example compounds listed below were added to enzyme/assay buffer mix and incubated 10 min. Substrate mix was added, and reaction mix was read in kinetic mode for 30 min at Ex480/Em540 and IC₅₀ response curves were plotted.

Data for all assay formats was calculated as percent inhibition compared with control wells. Percent inhibition was calculated using the following equation: % inhibition=100×[1−(X−min)/(max−min)], where X is the raw data readout, min is the average of the no enzyme control wells (n=32), max is the average of the DMSO control well (n=32). IC₅₀ values were calculated using the standard four parameter curve fitting algorithm in either Prism GraphPad (La Jolla, Calif.) software, or Collaborative Drug Discovery (Burlingame, Calif.) CDD Vault. See Chem. Biol. 20(1): 55-62 (Jan. 24, 2013); Bioorg. Med. Chem. Lett. 23(20): 5660-5666 (Oct. 15, 2013).

ES2 Ub-PCNA IF Assay

USP1 is a deubiquitinating enzyme that removes ubiquitin from mono-ubiquitinated

Proliferating Cell Nuclear Antigen (Ub-PCNA). The levels of Ub-PCNA in the nucleus of cells was used to assess the activity of USP1. An immunofluorescence assay was established to monitor Ub-PCNA levels in the ovarian cancer cell line, ES2.

This assay was performed by first plating 5000 ES2 cells per well in black 96 well plates (Corning #3904) and then incubated overnight at 37° C. and 5% CO₂. Compounds, resuspended in DMSO, were added to the cells, to a final DMSO concentration of 0.3%. Plates were incubated at 37° C. and 5% CO₂ for 3 hours.

Cells were then fixed and stained by first removing the media from each plate and fixing the cells with −20° C. methanol for 5 minutes at room temperature. Following fixation plates were washed with Tris buffered saline with tween (Boston Bioproducts ABB-855) 5 times for 5 minutes each. Plates were blocked for 1 hour with 50μ.1 of Odyssey blocking buffer (Licor #327-50000) at room temperature with rocking. Block was removed from all wells and 50 μl of primary antibody was added to each well. Ub-PCNA ab (Cell Signaling Technology #13439) was diluted at 1:400 in Odyssey blocking buffer. Plates were sealed and incubated overnight at 4° C. Primary antibodies were removed from the plates and plates were washed with Tris buffered saline with tween 5 times for 5 minutes. Plates were stained for 1 hour at room temperature with rocking with 50 μl of secondary antibodies diluted 1:10,000 in Odyssey blocking buffer at room temperature (anti-rabbit Alexa 488). Antibodies were removed from the plates and plates were washed with Tris buffered saline with tween 3 times for 5 minutes. Plates were washed once with DAPI (Chemometec # 910-3012) stain diluted 1:5000 (Stock solution of 500 μg/ml) in tris buffered saline with tween for 5 minutes. DAPI stain was removed and plates were washed one additional time for 5 minutes with tris buffered saline with tween. Wash was removed from the plate and 100 μl tris buffered saline with tween was added back to all wells, Plates were sealed with Foil seals or black plate seals and plates were stored at 4° C. until they could be imaged.

Imaging of the plates was performed by first locating the nuclei using the DAPI stain. A mask was created by drawing a circle slightly smaller than each nucleus. Ub-PCNA intensities were measured for each individual nucleus counted. A histogram of all Ub-PCNA nuclear intensities measured in untreated wells was generated and a 95% cutoff was established. This 95% cutoff was used to determine the number of Positive cells that had Ub-PCNA values higher than the 95% cutoff. The number of positive cells was expressed as a percentage by dividing by the total number of cells in the well. This percent positive value was used to graph all data and determine AC₅₀ values.

The following Compounds of the Disclosure inhibit USP1 activity with the IC₅₀ values shown in Table 34 below.

TABLE 34

| Example | | |
|---|---|---|
| Number | USP1 IC₅₀ (Ub-Rho) | USP1 IC₅₀ (Ub-PCNA) |
| 1 | +++ | ++ |
| 2 | +++ | ++ |
| 3 | ++ | No Data |
| 4 | +++ | No Data |
| 5 | ++ | No Data |
| 6 | +++ | + |
| 7 | +++ | ++ |
| 8 | ++ | No Data |
| 9 | +++ | +++ |
| 10 | +++ | ++ |
| 11 | + | + |
| 12 | +++ | ++ |
| 13 | ++ | No Data |
| 14 | +++ | +++ |
| 15 | + | No Data |
| 16 | +++ | +++ |
| 17 | +++ | No Data |
| 18 | +++ | +++ |
| 19 | +++ | No Data |
| 20 | +++ | +++ |
| 21 | ++ | No Data |
| 22 | +++ | +++ |
| 23 | ++ | No Data |
| 24 | +++ | +++ |
| 25 | ++ | + |
| 26 | ++ | No Data |
| 27 | +++ | +++ |
| 28 | +++ | +++ |
| 29 | +++ | +++ |
| 30 | +++ | +++ |
| 31 | ++ | No Data |
| 32 | +++ | No Data |
| 33 | +++ | +++ |
| 34 | ++ | No Data |
| 35 | ++ | + |
| 36 | +++ | No Data |
| 37 | +++ | +++ |
| 38 | +++ | +++ |
| 39 | +++ | +++ |
| 40 | +++ | ++ |
| 41 | +++ | +++ |
| 42 | +++ | +++ |
| 43 | +++ | ++ |
| 44 | +++ | +++ |
| 45 | + | No Data |
| 46 | ++ | ++ |
| 47 | +++ | +++ |
| 48 | +++ | +++ |
| 49 | +++ | +++ |
| 50 | +++ | +++ |
| 51 | +++ | +++ |
| 52 | +++ | No Data |
| 53 | +++ | ++ |
| 54 | +++ | +++ |
| 55 | +++ | +++ |
| 56 | +++ | No Data |
| 57 | +++ | +++ |
| 58 | +++ | +++ |
| 59 | +++ | +++ |
| 60 | +++ | No Data |
| 61 | +++ | +++ |
| 62 | +++ | +++ |
| 63 | +++ | +++ |
| 64 | +++ | +++ |
| 65 | +++ | +++ |
| 66 | +++ | +++ |
| 67 | +++ | +++ |
| 68 | +++ | +++ |
| 69 | ++ | No Data |
| 70 | ++ | + |
| 71 | +++ | +++ |
| 72 | +++ | ++ |
| 73 | +++ | ++ |
| 74 | +++ | + |
| 75 | +++ | ++ |
| 76 | ++ | No Data |
| 77 | +++ | No Data |
| 78 | +++ | +++ |
| 79 | +++ | +++ |
| 80 | +++ | ++ |
| 81 | +++ | No Data |
| 82 | ++ | No Data |
| 83 | + | No Data |
| 84 | ++ | No Data |
| 85 | ++ | No Data |
| 86 | +++ | ++ |
| 87 | + | No Data |
| 88 | ++ | No Data |
| 89 | +++ | No Data |
| 90 | + | No Data |
| 91 | +++ | +++ |
| 92 | + | No Data |
| 93 | +++ | No Data |
| 94 | ++ | No Data |
| 95 | ++ | No Data |
| 96 | +++ | +++ |
| 97 | +++ | +++ |
| 98 | +++ | +++ |
| 99 | ++ | No Data |
| 100 | +++ | No Data |
| 101 | ++ | No Data |
| 102 | ++ | No Data |
| 103 | +++ | ++ |
| 104 | +++ | ++ |
| 105 | ++ | No Data |
| 106 | +++ | No Data |
| 107 | ++ | No Data |
| 108 | +++ | +++ |
| 109 | +++ | +++ |
| 110 | ++ | No Data |
| 111 | +++ | ++ |
| 112 | +++ | No Data |
| 113 | +++ | +++ |
| 114 | +++ | ++ |
| 115 | +++ | +++ |
| 116 | +++ | ++ |
| 117 | ++ | No Data |
| 118 | +++ | +++ |
| 119 | +++ | +++ |
| 120 | +++ | +++ |
| 121 | ++ | No Data |
| 122 | ++ | + |
| 123 | +++ | +++ |
| 124 | ++ | + |
| 125 | +++ | +++ |
| 126 | +++ | ++ |
| 127 | +++ | +++ |
| 128 | ++ | No Data |
| 129 | +++ | +++ |
| 130 | ++ | No Data |
| 131 | +++ | +++ |
| 132 | +++ | +++ |
| 133 | +++ | ++ |
| 134 | ++ | No Data |
| 135 | +++ | +++ |
| 136 | ++ | No Data |
| 137 | ++ | No Data |
| 138 | ++ | No Data |
| 139 | +++ | ++ |
| 140 | +++ | +++ |
| 141 | +++ | +++ |
| 142 | ++ | ++ |
| 143 | +++ | +++ |
| 144 | +++ | +++ |
| 145 | +++ | +++ |
| 146 | ++ | No Data |
| 147 | +++ | +++ |
| 148 | +++ | +++ |
| 149 | ++ | No Data |
| 150 | ++ | No Data |
| 151 | +++ | +++ |
| 152 | ++ | No Data |
| 153 | +++ | ++ |
| 154 | +++ | No Data |
| 155 | ++ | No Data |
| 156 | +++ | No Data |
| 157 | ++ | No Data |
| 158 | + | No Data |
| 159 | +++ | ++ |
| 160 | +++ | ++ |
| 161 | ++ | No Data |
| 162 | +++ | ++ |
| 163 | ++ | No Data |
| 164 | +++ | +++ |
| 165 | +++ | ++ |
| 166 | ++ | ++ |
| 167 | ++ | No Data |
| 168 | +++ | +++ |
| 169 | +++ | ++ |
| 170 | +++ | +++ |
| 171 | +++ | +++ |
| 172 | +++ | ++ |
| 173 | +++ | +++ |
| 174 | +++ | +++ |
| 175 | +++ | +++ |
| 176 | +++ | +++ |
| 177 | +++ | +++ |
| 178 | +++ | +++ |
| 179 | ++ | No Data |
| 180 | +++ | ++ |
| 181 | +++ | +++ |
| 182 | +++ | +++ |
| 183 | +++ | +++ |
| 184 | +++ | +++ |
| 185 | ++ | No Data |
| 186 | +++ | ++ |
| 187 | ++ | No Data |
| 188 | ++ | +++ |
| 189 | +++ | +++ |
| 190 | +++ | +++ |
| 191 | +++ | +++ |
| 192 | ++ | No Data |
| 193 | +++ | +++ |
| 194 | +++ | +++ |
| 195 | ++ | +++ |
| 196 | ++ | +++ |
| 197 | +++ | +++ |
| 198 | + | No Data |
| 199 | ++ | No Data |
| 200 | ++ | +++ |
| 201 | +++ | +++ |
| 202 | +++ | No Data |
| 203 | ++ | ++ |
| 204 | +++ | +++ |
| 205 | +++ | No Data |
| 206 | +++ | +++ |
| 207 | ++ | No Data |
| 208 | +++ | No Data |
| 209 | +++ | No Data |
| 210 | +++ | +++ |
| 211 | +++ | +++ |
| 212 | +++ | +++ |
| 213 | +++ | ++ |
| 214 | +++ | No Data |
| 215 | ++ | No Data |
| 216 | +++ | ++ |
| 217 | +++ | +++ |
| 218 | ++ | No Data |
| 219 | +++ | No Data |
| 220 | +++ | No Data |
| 221 | +++ | +++ |
| 222 | +++ | +++ |
| 223 | +++ | No Data |
| 224 | ++ | No Data |
| 225 | ++ | No Data |
| 226 | ++ | No Data |
| 227 | ++ | No Data |
| 228 | ++ | No Data |
| 229 | ++ | No Data |
| 230 | + | No Data |
| 231 | ++ | No Data |
| 232 | ++ | ++ |
| 233 | +++ | ++ |
| 234 | ++ | No Data |
| 235 | +++ | ++ |
| 236 | ++ | + |
| 237 | +++ | +++ |
| 238 | +++ | +++ |
| 239 | +++ | +++ |
| 240 | +++ | +++ |
| 241 | +++ | ++ |
| 242 | +++ | ++ |
| 243 | +++ | ++ |
| 244 | +++ | +++ |
| 245 | +++ | ++ |
| 246 | +++ | ++ |
| 247 | +++ | ++ |
| 248 | +++ | +++ |
| 249 | +++ | +++ |
| 250 | +++ | +++ |
| 251 | + | No Data |
| 252 | ++ | No Data |
| 253 | ++ | No Data |
| 254 | ++ | No Data |
| 255 | +++ | ++ |
| 256 | ++ | No Data |
| 257 | ++ | No Data |
| 258 | +++ | ++ |
| 259 | ++ | No Data |
| 260 | ++ | No Data |
| 261 | +++ | +++ |
| 262 | +++ | ++ |
| 263 | +++ | +++ |
| 264 | +++ | ++ |
| 265 | +++ | ++ |
| 266 | +++ | ++ |
| 267 | +++ | +++ |
| 268 | + | No Data |
| 269 | + | No Data |
| 270 | +++ | +++ |
| 271 | ++ | No Data |
| 272 | + | No Data |
| 273 | + | No Data |
| 274 | ++ | No Data |
| 275 | ++ | No Data |
| 276 | ++ | No Data |
| 277 | + | No Data |
| 278 | + | No Data |
| 279 | + | No Data |
| 280 | + | No Data |
| 281 | + | No Data |
| 282 | + | No Data |
| 283 | + | No Data |
| 284 | + | No Data |
| 285 | + | No Data |
| 286 | + | No Data |
| 287 | + | No Data |
| 288 | +++ | No Data |
| 289 | + | No Data |
| 290 | +++ | ++ |
| 291 | +++ | ++ |
| 292 | ++ | No Data |
| 293 | + | No Data |
| 294 | +++ | +++ |
| 295 | ++ | No Data |
| 296 | +++ | ++ |
| 297 | ++ | No Data |
| 298 | ++ | No Data |
| 299 | + | No Data |
| 300 | ++ | No Data |
| 301 | ++ | No Data |
| 302 | ++ | No Data |
| 303 | ++ | No Data |
| 304 | +++ | ++ |
| 305 | +++ | ++ |
| 306 | ++ | No Data |
| 307 | +++ | ++ |
| 308 | ++ | No Data |
| 309 | +++ | ++ |
| 310 | +++ | No Data |
| 311 | + | No Data |
| 312 | +++ | No Data |
| 313 | ++ | No Data |
| 314 | + | No Data |
| 315 | + | No Data |
| 316 | ++ | No Data |
| 317 | ++ | No Data |
| 318 | + | No Data |
| 319 | + | No Data |
| 320 | ++ | No Data |
| 321 | +++ | +++ |
| 322 | ++ | No Data |
| 323 | +++ | No Data |
| 324 | +++ | No Data |
| 325 | ++ | ++ |
| 326 | ++ | No Data |
| 327 | +++ | +++ |
| 328 | ++ | No Data |
| 329 | ++ | No Data |
| 330 | +++ | +++ |
| 331 | +++ | +++ |
| 332 | +++ | +++ |
| 333 | +++ | +++ |
| 334 | +++ | ++ |
| 335 | +++ | +++ |
| 336 | +++ | +++ |
| 337 | +++ | +++ |
| 338 | +++ | +++ |
| 339 | +++ | +++ |
| 340 | +++ | +++ |
| 341 | +++ | No Data |
| 342 | +++ | No Data |
| 343 | +++ | +++ |
| 344 | +++ | No Data |
| 345 | +++ | +++ |
| 346 | +++ | +++ |
| 347 | +++ | +++ |
| 348 | +++ | +++ |
| 349 | +++ | +++ |
| 350 | +++ | +++ |
| 351 | +++ | +++ |
| 352 | +++ | +++ |
| 353 | +++ | +++ |
| 354 | + | No Data |
| 355 | +++ | ++ |
| 356 | ++ | No Data |
| 357 | +++ | +++ |
| 358 | +++ | +++ |
| 359 | ++ | No Data |
| 360 | +++ | +++ |
| 361 | ++ | No Data |
| 362 | +++ | ++ |
| 364 | +++ | +++ |
| 365 | +++ | +++ |
| 366 | +++ | No Data |
| 367 | +++ | No Data |
| 368 | +++ | No Data |
| 369 | +++ | No Data |
| 370 | +++ | +++ |
| 371 | +++ | +++ |
| 372 | +++ | No Data |
| 373 | ++ | No Data |
| 374 | +++ | No Data |
| 375 | +++ | No Data |
| 376 | +++ | No Data |
| 377 | +++ | No Data |
| 378 | +++ | No Data |
| 381 | +++ | No Data |
| 382 | +++ | No Data |
| 383 | ++ | No Data |
| 384 | +++ | No Data |
| 385 | +++ | No Data |
| 386 | +++ | No Data |
| 387 | +++ | No Data |
| 388 | +++ | No Data |
| 389 | +++ | No Data |
| 390 | +++ | No Data |
| 391 | +++ | No Data |
| 392 | +++ | No Data |
| 393 | +++ | No Data |

USP1 IC₅₀: “+” indicates greater than 500 nM; “++” indicates 100 nM to 500 nM; “+++” indicates less than 100 nM.

### Example 395

### Solubility Determination

Certain Compounds of the Disclosure were assessed for ADME solubility at pH 2.0 and pH 7.4.

Stock solutions were prepared by adding each compound to DMSO at a concentration of 10 mM. Samples were prepared by adding 50 μL of each stock solution to separate vials. The vials were loaded onto a 96-well rack and dried. 500 μL of Phosphate Buffered Saline (PBS) pH 7.4 or PBS pH 2.0 were added into each vial. The vials were then shaken at 25° C. and 1,100 rpm for 24 hours.

After 24 hours, the vials were centrifuged at 3220 G and 25° C. for 30 minutes. The supernatant fluid was analyzed by LC-MS/MS against a standard of known concentration. The solubility of each sample was then calculated using the equation below:

$$[ Sample ] = \frac{1}{n} * Σ ( Calculated Conc . sample \times DF sample )$$

where DF is the dilution factor.

The following Compounds of the Disclosure have the ADME solubility values shown in

Table 36 below.

TABLE 36

| | Solubility | Solubility |
|---|---|---|
| Example Number | at pH 2.0 (μM) | at pH 7.4 (μM) |
| 1 | ++ | + |
| 2 | + | + |
| 4 | + | + |
| 6 | ++ | ++ |
| 7 | + | + |
| 8 | ++ | + |
| 9 | ++ | ++ |
| 10 | + | + |
| 11 | ++ | + |
| 12 | + | + |
| 13 | ++ | ++ |
| 14 | + | + |
| 16 | + | + |
| 18 | + | + |
| 19 | + | + |
| 20 | + | + |
| 22 | ++ | + |
| 24 | ++ | ++ |
| 25 | ++ | ++ |
| 27 | ++ | + |
| 28 | ++ | + |
| 29 | + | + |
| 30 | ++ | + |
| 31 | ++ | + |
| 32 | ++ | + |
| 33 | + | + |
| 34 | + | + |
| 35 | + | + |
| 36 | ++ | ++ |
| 37 | + | + |
| 38 | + | + |
| 39 | + | + |
| 40 | + | + |
| 41 | + | + |
| 42 | + | + |
| 43 | ++ | + |
| 44 | + | + |
| 46 | ++ | ++ |
| 47 | ++ | + |
| 48 | ++ | ++ |
| 49 | ++ | + |
| 50 | + | + |
| 51 | ++ | + |
| 52 | + | + |
| 53 | + | + |
| 54 | ++ | + |
| 55 | ++ | + |
| 56 | + | + |
| 57 | + | + |
| 58 | ++ | + |
| 59 | + | + |
| 60 | + | + |
| 61 | + | + |
| 62 | + | + |
| 63 | ++ | + |
| 64 | ++ | + |
| 65 | + | + |
| 66 | ++ | + |
| 67 | ++ | + |
| 68 | + | + |
| 70 | + | + |
| 71 | + | + |
| 72 | + | + |
| 73 | + | + |
| 74 | ++ | + |
| 75 | ++ | + |
| 77 | + | + |
| 78 | + | + |
| 79 | + | + |
| 80 | + | + |
| 86 | + | + |
| 91 | + | + |
| 93 | + | + |
| 96 | + | + |
| 97 | + | + |
| 98 | + | + |
| 100 | + | + |
| 103 | + | + |
| 104 | + | + |
| 106 | + | + |
| 108 | ++ | + |
| 109 | + | + |
| 111 | + | + |
| 112 | + | + |
| 113 | + | + |
| 114 | ++ | ++ |
| 115 | + | + |
| 116 | + | + |
| 118 | + | + |
| 119 | + | + |
| 120 | + | + |
| 122 | ++ | + |
| 123 | ++ | + |
| 124 | ++ | ++ |
| 125 | ++ | + |
| 126 | + | + |
| 127 | + | + |
| 128 | ++ | + |
| 129 | ++ | + |
| 130 | + | + |
| 131 | + | + |
| 132 | + | + |
| 133 | + | + |
| 135 | + | + |
| 138 | + | + |
| 139 | + | + |
| 140 | ++ | + |
| 141 | + | + |
| 142 | ++ | + |
| 143 | ++ | ++ |
| 144 | ++ | + |
| 145 | + | + |
| 146 | + | + |
| 147 | + | + |
| 148 | + | + |
| 151 | + | + |
| 153 | + | + |
| 154 | + | + |
| 156 | + | + |
| 159 | + | + |
| 160 | + | + |
| 162 | ++ | ++ |
| 164 | + | + |
| 165 | + | + |
| 166 | ++ | ++ |
| 167 | + | + |
| 168 | + | + |
| 169 | ++ | + |
| 170 | ++ | ++ |
| 171 | ++ | ++ |
| 172 | ++ | ++ |
| 173 | ++ | ++ |
| 174 | ++ | ++ |
| 175 | ++ | ++ |
| 176 | ++ | ++ |
| 177 | + | + |
| 178 | ++ | + |
| 179 | ++ | + |
| 180 | + | + |
| 181 | + | + |
| 182 | + | + |
| 183 | ++ | ++ |
| 184 | ++ | ++ |
| 186 | + | + |
| 187 | + | + |
| 188 | ++ | ++ |
| 189 | ++ | ++ |
| 190 | ++ | ++ |
| 191 | + | + |
| 193 | + | + |
| 194 | + | + |
| 195 | ++ | ++ |
| 196 | ++ | ++ |
| 197 | ++ | ++ |
| 200 | ++ | ++ |
| 201 | ++ | ++ |
| 202 | + | + |
| 203 | ++ | ++ |
| 204 | ++ | ++ |
| 205 | + | ++ |
| 206 | ++ | + |
| 207 | ++ | + |
| 208 | ++ | ++ |
| 209 | ++ | + |
| 210 | ++ | ++ |
| 211 | ++ | + |
| 212 | ++ | ++ |
| 213 | + | + |
| 216 | + | + |
| 217 | ++ | ++ |
| 221 | ++ | ++ |
| 222 | ++ | + |
| 223 | + | + |
| 231 | + | + |
| 232 | + | + |
| 233 | ++ | + |
| 235 | ++ | + |
| 236 | ++ | + |
| 237 | + | + |
| 238 | + | + |
| 239 | + | + |
| 240 | ++ | + |
| 241 | + | + |
| 242 | + | + |
| 243 | ++ | + |
| 244 | + | + |
| 245 | + | + |
| 246 | ++ | + |
| 247 | + | + |
| 248 | ++ | ++ |
| 249 | + | + |
| 250 | + | + |
| 255 | + | + |
| 257 | + | + |
| 258 | + | + |
| 261 | + | + |
| 262 | + | + |
| 263 | + | + |
| 264 | ++ | + |
| 265 | + | + |
| 266 | + | + |
| 267 | + | + |
| 270 | + | + |
| 288 | ++ | + |
| 290 | ++ | + |
| 291 | + | + |
| 294 | + | + |
| 296 | ++ | + |
| 304 | ++ | + |
| 305 | ++ | + |
| 307 | + | + |
| 309 | ++ | + |
| 310 | ++ | + |
| 312 | ++ | + |
| 321 | ++ | + |
| 323 | + | + |
| 324 | + | + |
| 325 | + | + |
| 327 | ++ | + |
| 328 | ++ | + |
| 330 | ++ | + |
| 331 | + | + |
| 332 | ++ | ++ |
| 333 | + | + |
| 334 | + | + |
| 335 | ++ | + |
| 336 | ++ | + |
| 337 | ++ | + |
| 338 | ++ | + |
| 339 | ++ | + |
| 340 | ++ | ++ |
| 341 | ++ | ++ |
| 342 | ++ | ++ |
| 343 | + | + |
| 345 | + | + |
| 346 | + | + |
| 347 | ++ | ++ |
| 348 | ++ | ++ |
| 349 | ++ | + |
| 350 | + | + |
| 351 | + | + |
| 352 | + | + |
| 353 | ++ | + |
| 355 | + | + |
| 357 | + | + |
| 358 | + | + |
| 360 | ++ | + |
| 362 | ++ | ++ |
| 364 | + | + |
| 365 | + | + |
| 370 | + | + |
| 371 | + | + |

Solubility: “+” indicates less than 10 μM; “++” indicates 10 μM or greater.

### Example 396

### Liver Microsomal Stability

Certain Compounds of the Disclosure were assessed for ADME metabolic stability in human liver microsomes (HLM) and rat liver microsomes (RLM).

Samples were prepared by adding 222.5 μL of a master solution (100 mM phosphate buffer and 1 mg/mL liver microsomes (HLM or RLM)) and 25 μL of a 10 mM NADPH solution to incubation plates, which were then warmed for 10 min. Each compound was separately dissolved in DMSO to prepare 10 mM stock solutions, which were then diluted to 100 μM with acetonitrile. A reaction was started by adding 2.5 μL of the 100 μM solution of each compound to separate incubation plates such that the final concentration of for each compound in each plate was 1 μM.

25 μL aliquots of each sample were taken at 0.5, 5, 10, 15, 20 and 30 minutes, and the reaction was stopped by adding 5 volumes of cold acetonitrile with IS (100 nM alprazolam, 200 nM caffeine and 100 nM tolbutamide). The samples were then centrifuged at 3,220 G for 30 minutes, and 100 μL of the supernatant fluid was mixed with 100 μL of ultra-pure H₂O.

The samples were then analyzed by LC-MS/MS. Peak areas were determined from extracted ion chromatograms. Slope values (k) were determined by linear regression of the natural logarithm of the remaining percentage of the compound vs. incubation time curve. The in vitro half-life (in vitro t_{1/2}) was determined from the slope value using the following equation:

in vitro t_{1/2}=−(0.693/k).

The in vitro half-life (min) was converted into the in vitro intrinsic clearance (in vitro CLᵢₙₜ, in μL/min/mg protein) using the following equation:

$$in vitro {CL}_{int} = ( \frac{0.693}{( {t}_{1 / 2} )} ) * ( \frac{volume of incubation ( μL )}{amount of proteins ( mg )} )$$

The following Compounds of the Disclosure have the ADME metabolic stability values shown in Table 37 below.

TABLE 37

| Example Number | t_{1/2} (min) in HLM | t_{1/2} (min) in RLM |
|---|---|---|
| 1 | ++ | ++ |
| 2 | ++ | ++ |
| 4 | ++ | ++ |
| 6 | + | + |
| 7 | ++ | ++ |
| 8 | ++ | ++ |
| 9 | + | + |
| 10 | + | + |
| 11 | + | + |
| 12 | + | + |
| 13 | + | + |
| 14 | ++ | ++ |
| 16 | ++ | + |
| 18 | ++ | ++ |
| 19 | + | + |
| 20 | ++ | ++ |
| 22 | + | + |
| 24 | + | + |
| 25 | + | + |
| 27 | + | + |
| 28 | + | + |
| 29 | + | + |
| 30 | + | + |
| 31 | + | + |
| 32 | ++ | ++ |
| 33 | ++ | ++ |
| 34 | + | + |
| 35 | + | ++ |
| 36 | + | + |
| 37 | + | + |
| 38 | + | + |
| 39 | ++ | ++ |
| 40 | ++ | ++ |
| 41 | ++ | ++ |
| 42 | ++ | ++ |
| 43 | ++ | ++ |
| 44 | ++ | ++ |
| 46 | ++ | ++ |
| 47 | + | + |
| 48 | + | + |
| 49 | + | + |
| 50 | + | + |
| 51 | + | + |
| 52 | + | + |
| 53 | + | + |
| 54 | + | + |
| 55 | + | + |
| 56 | + | + |
| 57 | + | + |
| 58 | + | + |
| 59 | + | + |
| 60 | + | + |
| 61 | + | + |
| 62 | + | + |
| 63 | + | + |
| 64 | + | + |
| 65 | + | + |
| 66 | + | + |
| 67 | + | + |
| 68 | + | + |
| 70 | + | + |
| 71 | ++ | ++ |
| 72 | ++ | ++ |
| 73 | + | + |
| 74 | + | + |
| 75 | + | + |
| 77 | ++ | + |
| 78 | ++ | ++ |
| 79 | ++ | ++ |
| 80 | ++ | ++ |
| 86 | ++ | ++ |
| 91 | ++ | ++ |
| 93 | + | + |
| 96 | ++ | ++ |
| 97 | ++ | ++ |
| 98 | ++ | ++ |
| 100 | + | + |
| 103 | ++ | ++ |
| 104 | ++ | ++ |
| 106 | ++ | ++ |
| 108 | ++ | ++ |
| 109 | ++ | ++ |
| 111 | ++ | ++ |
| 112 | ++ | + |
| 113 | + | + |
| 114 | ++ | + |
| 115 | ++ | ++ |
| 116 | ++ | ++ |
| 118 | + | + |
| 119 | ++ | + |
| 120 | ++ | ++ |
| 122 | ++ | ++ |
| 123 | ++ | ++ |
| 124 | + | + |
| 125 | ++ | ++ |
| 126 | + | + |
| 127 | ++ | ++ |
| 128 | + | + |
| 129 | ++ | + |
| 130 | ++ | ++ |
| 131 | + | + |
| 132 | + | + |
| 133 | ++ | ++ |
| 135 | + | + |
| 138 | + | + |
| 139 | ++ | ++ |
| 140 | ++ | ++ |
| 141 | ++ | ++ |
| 142 | ++ | + |
| 143 | ++ | ++ |
| 144 | ++ | ++ |
| 145 | + | + |
| 146 | + | + |
| 147 | + | + |
| 148 | + | + |
| 151 | + | + |
| 153 | + | + |
| 154 | ++ | ++ |
| 156 | ++ | ++ |
| 159 | ++ | + |
| 160 | + | + |
| 162 | n/a | + |
| 164 | ++ | ++ |
| 165 | ++ | ++ |
| 166 | ++ | ++ |
| 168 | ++ | ++ |
| 169 | ++ | ++ |
| 170 | ++ | ++ |
| 171 | ++ | ++ |
| 172 | ++ | ++ |
| 173 | ++ | ++ |
| 174 | ++ | ++ |
| 175 | ++ | ++ |
| 176 | ++ | ++ |
| 177 | + | ++ |
| 178 | + | + |
| 179 | + | + |
| 180 | + | + |
| 181 | + | ++ |
| 182 | + | + |
| 183 | + | ++ |
| 184 | ++ | + |
| 186 | ++ | ++ |
| 187 | ++ | + |
| 188 | ++ | ++ |
| 189 | ++ | ++ |
| 190 | ++ | ++ |
| 191 | ++ | + |
| 193 | + | + |
| 194 | + | + |
| 195 | ++ | ++ |
| 196 | + | ++ |
| 197 | ++ | ++ |
| 200 | ++ | ++ |
| 201 | ++ | ++ |
| 202 | ++ | ++ |
| 203 | ++ | ++ |
| 204 | ++ | ++ |
| 205 | + | ++ |
| 206 | ++ | ++ |
| 207 | + | ++ |
| 208 | ++ | + |
| 209 | + | + |
| 210 | ++ | ++ |
| 211 | ++ | + |
| 212 | ++ | ++ |
| 213 | ++ | ++ |
| 216 | ++ | ++ |
| 217 | ++ | ++ |
| 221 | + | + |
| 222 | + | + |
| 223 | + | ++ |
| 231 | ++ | ++ |
| 232 | ++ | ++ |
| 233 | ++ | ++ |
| 235 | + | + |
| 236 | + | + |
| 237 | ++ | + |
| 238 | ++ | ++ |
| 239 | ++ | ++ |
| 240 | ++ | ++ |
| 241 | ++ | ++ |
| 242 | ++ | ++ |
| 243 | ++ | ++ |
| 244 | ++ | ++ |
| 245 | ++ | ++ |
| 246 | ++ | ++ |
| 247 | + | + |
| 248 | ++ | ++ |
| 249 | ++ | ++ |
| 250 | ++ | ++ |
| 255 | ++ | + |
| 257 | ++ | ++ |
| 258 | + | + |
| 261 | ++ | ++ |
| 262 | ++ | ++ |
| 263 | ++ | ++ |
| 264 | ++ | ++ |
| 265 | + | + |
| 266 | ++ | ++ |
| 267 | + | + |
| 270 | + | + |
| 288 | + | + |
| 290 | + | + |
| 291 | + | + |
| 294 | ++ | + |
| 296 | + | + |
| 304 | + | + |
| 305 | + | + |
| 307 | + | + |
| 309 | + | + |
| 310 | + | + |
| 312 | + | + |
| 321 | + | + |
| 323 | ++ | ++ |
| 324 | ++ | ++ |
| 325 | ++ | ++ |
| 327 | + | + |
| 328 | ++ | ++ |
| 330 | + | + |
| 331 | + | + |
| 332 | ++ | ++ |
| 333 | ++ | ++ |
| 334 | ++ | ++ |
| 335 | ++ | ++ |
| 336 | + | ++ |
| 337 | ++ | ++ |
| 338 | ++ | + |
| 339 | + | ++ |
| 340 | ++ | ++ |
| 341 | ++ | ++ |
| 342 | ++ | ++ |
| 343 | ++ | ++ |
| 345 | ++ | ++ |
| 346 | ++ | ++ |
| 347 | ++ | ++ |
| 348 | ++ | ++ |
| 349 | ++ | ++ |
| 350 | ++ | ++ |
| 351 | ++ | ++ |
| 352 | ++ | + |
| 353 | + | + |
| 355 | + | + |
| 357 | + | + |
| 358 | ++ | + |
| 360 | + | + |
| 362 | + | + |
| 364 | ++ | ++ |
| 365 | ++ | ++ |
| 370 | ++ | ++ |
| 371 | + | + |

HLM/RLM Stability t_{1/2}: “+” indicates less than 25 minutes; “++” indicates 25 minutes greater.

Having now fully described this invention, it will be understood by those of ordinary skill in the art that the same can be performed within a wide and equivalent range of conditions, formulations, and other parameters without affecting the scope of the invention or any embodiment thereof.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

All patents and publications cited herein are fully incorporated by reference herein in their entirety.

What is claimed is:

1. A compound having Formula I: 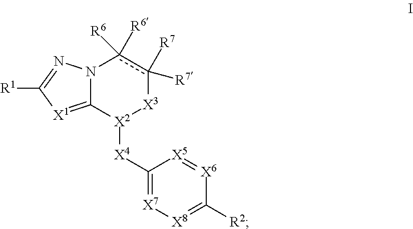
or a pharmaceutically acceptable salt or solvate thereof, wherein:
X¹ is selected from N and CR⁵;
X² is selected from N and CH;
X³ is selected from NR³, O, and CR³R^{3′};
X⁴ is selected from NR⁴, O, and CR⁴R^{4′};
each of X⁵, X⁶, X⁷, and X⁸ are independently selected from N and CR⁸;
R¹ is selected from optionally substituted C₆ aryl and optionally substituted 5- or 6-membered nitrogen-containing heteroaryl;
R² is selected from hydrogen, halo, alkoxy, optionally substituted (C₆-C₁₄) aryl, optionally substituted (C₆-C₁₄) ar-(C₁-C₂) alkyl, optionally substituted nitrogen-containing heteroaryl, optionally substituted, nitrogen-containing heteroar-(C₁-C₂) alkyl, optionally substituted (C₃-C₈) cycloalkyl, optionally substituted ((C₃-C₈) cycloalkyl)-(C₁-C₂) alkyl, optionally substituted heterocyclo, optionally substituted heterocyclo-(C₁-C₂) alkyl, optionally substituted (C₆-C₁₄) aryloxy, optionally substituted (C₆-C₁₄) ar-(C₁-C₂) alkyloxy, optionally substituted nitrogen-containing heteroaryloxy, optionally substituted nitrogen-containing heteroar-(C₁-C₂) alkyloxy, optionally substituted (C₃-C₈) cycloalkyloxy, optionally substituted ((C₃-C₈) cycloalkyl)-(C₁-C₂) alkyloxy, optionally substituted heterocyclooxy, optionally substituted heterocyclo-(C₁-C₂) alkyloxy;
each of R³ and R^{3′} is independently selected from hydrogen, halo, cyano, amino, hydroxy, alkoxy, optionally substituted alkyl, optionally substituted alkenyl, and optionally substituted alkynyl, and optionally substituted cycloalkyl; or
R³ and R^{3′} are taken together with the atom to which they are attached to form a carbonyl or an optionally substituted cycloalkyl;
each of R⁴ and R^{4′} is independently selected from hydrogen, halo, cyano, amino, optionally substituted alkyl, optionally substituted alkenyl, and optionally substituted alkynyl, and optionally substituted cycloalkyl;
R⁵ is selected from hydrogen, halo, cyano, amino, alkylamino, dialkylamino, hydroxy, alkoxy, optionally substituted alkyl, optionally substituted alkenyl, and optionally substituted alkynyl, and optionally substituted cycloalkyl;
each of R⁶ and R^{6′}, if present, is independently selected from hydrogen, halo, cyano, amino, alkylamino, dialkylamino, hydroxy, alkoxy, optionally substituted alkyl, optionally substituted alkenyl, and optionally substituted alkynyl, and optionally substituted cycloalkyl, and alkylcarbonylamino;
each of R₇ and R^{7′}, if present, is independently selected from hydrogen, halo, cyano, amino, alkylamino, dialkylamino, hydroxy, alkoxy, optionally substituted alkyl, optionally substituted alkenyl, and optionally substituted alkynyl, optionally substituted cycloalkyl, and alkylcarbonylamino; or
R⁷ and R^{7′} are taken together with the atom to which they are attached to form a carbonyl or an optionally substituted cycloalkyl; and
R⁸ is selected from hydrogen, halo, cyano, amino, alkylamino, dialkylamino, hydroxy, and alkoxy; or
R⁸ and R⁴ or R^{4′} are taken together with the atoms to which they are attached to form an optionally substituted cycloalkyl.

2. The compound of claim 1, or a pharmaceutically acceptable salt or solvate thereof, wherein 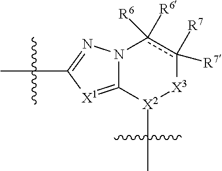
is independently selected from

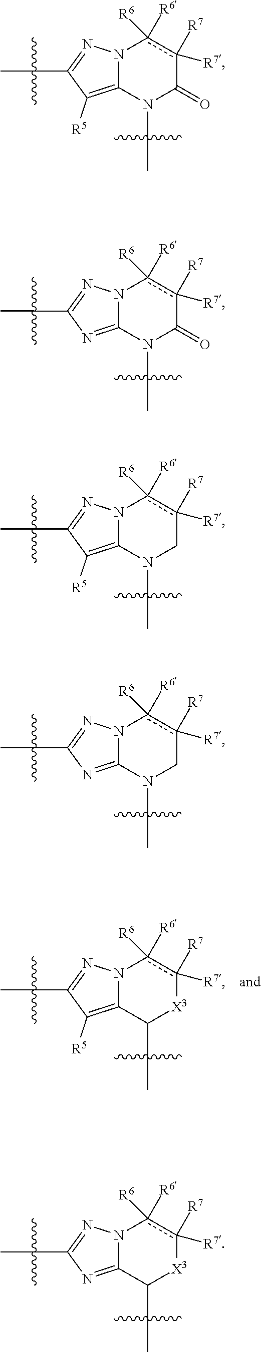

3. The compound of claim 2, or a pharmaceutically acceptable salt or solvate thereof, wherein 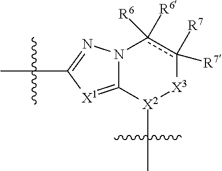
is independently selected from

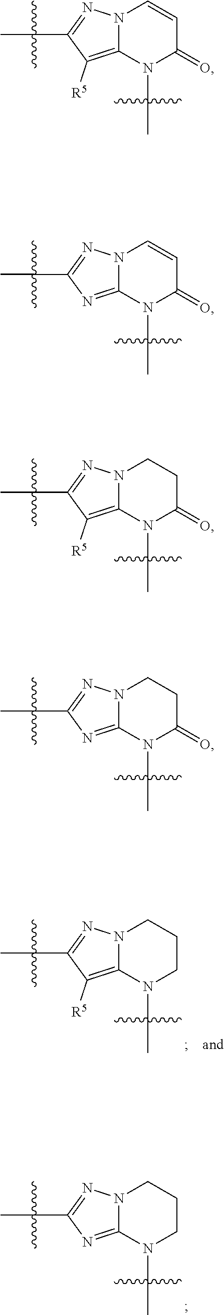

4. The compound of claim 1, or a pharmaceutically acceptable salt or solvate thereof, wherein X⁴ is CR⁴R^{4′} and X² is N.

5. The compound of claim 1, or a pharmaceutically acceptable salt or solvate thereof, wherein X⁴ is NR⁴ and X² is CH.

6. The compound of claim 1, or a pharmaceutically acceptable salt or solvate thereof, wherein one of X⁵, X⁶, X⁷, and X⁸ is N.

7. The compound of claim 1, or a pharmaceutically acceptable salt or solvate thereof, wherein two of X⁵, X⁶, X⁷, and X⁸ is N.

8. The compound of claim 1, or a pharmaceutically acceptable salt or solvate thereof, wherein X⁶ is CR⁸.

9. The compound of claim 8, or a pharmaceutically acceptable salt or solvate thereof, wherein R⁸ is selected from hydrogen and fluoro.

10. The compound of claim 1, or a pharmaceutically acceptable salt or solvate thereof, wherein the optional substituents on R¹ are independently selected from hydrogen, halo, nitro, cyano, hydroxy, amino, alkylamino, dialkylamino, haloalkyl, hydroxyalkyl, alkoxy, haloalkoxy, aryloxy, heteroaryloxy, aralkyl aralkyloxy, alkylthio, carboxamido, sulfonamido, alkylcarbonyl, arylcarbonyl, alkyl sulfonyl, arylsulfonyl, carboxy, carboxyalkyl, alkyl, optionally substituted cycloalkyl, alkenyl, alkynyl, optionally substituted aryl, optionally substituted heteroaryl, optionally substituted heterocyclo, alkoxyalkyl, (amino)alkyl, hydroxyalkylamino, (alkylamino)alkyl, (dialkylamino)alkyl, (cyano)alkyl, (carboxamido)alkyl, mercaptoalkyl, (heterocyclo)alkyl, (cycloalkylamino)alkyl, (C₁₋₄haloalkoxy)alkyl, and (heteroaryl)alkyl; or wherein two of the optional substituents on R¹ are taken together with the carbon or nitrogen atoms to which they are attached to form an optionally substituted cycloalkyl, optionally substituted heterocyclo, optionally substituted aryl, or optionally substituted heteroaryl group.

11. The compound of any of claims 1-10, or a pharmaceutically acceptable salt or solvate thereof, wherein R¹ is an optionally substituted 5- or 6-membered nitrogen-containing heteroaryl, wherein nitrogen is the only heteroatom.

12. The compound of any of claims 1-10, or a pharmaceutically acceptable salt or solvate thereof, wherein R¹ is an optionally substituted pyrimidin-5-yl, and wherein the pyrimidin-5-yl is optionally substituted at the 4-position, optionally substituted at the 6-position, optionally disubstituted at the 4- and 6-positions, or optionally trisubstituted at the 2-, 4-, and 6-positions.

13. The compound of any of claims 1-10, or a pharmaceutically acceptable salt or solvate thereof, wherein R¹ is an optionally substituted pyrazol-5-yl, wherein the pyrazol-5-yl is optionally substituted at the 1-position, optionally substituted at the 4-position, or optionally disubstituted at the 1- and 4-positions.

14. The compound of any of claims 1-10, or a pharmaceutically acceptable salt or solvate thereof, wherein R¹ is an optionally substituted pyrid-3-yl or optionally substituted pyrid-4-yl, wherein the pyrid-3-yl is optionally substituted at the 2-position, optionally substituted at the 4-position, or optionally disubstituted at the 2- and 4-positions; and
wherein the pyrid-4-yl is optionally substituted at the 3-position, optionally substituted at the 5-position, or optionally disubstituted at the 3- and 5-positions.

15. The compound of any of claims 1-10, or a pharmaceutically acceptable salt or solvate thereof, wherein R¹ is an optionally substituted phenyl, wherein the phenyl is optionally substituted at the 2-position, optionally substituted at the 6-position, optionally disubstituted at the 2- and 6-positions, or optionally disubstituted at the 2- and 3-positions.

16. The compound of any one of claims 1-15, wherein R¹ is substituted and the substituents are independently selected from hydrogen, cyano, amino, methylamino, dimethylamino, methoxy, ethoxy, isopropoxy, tert-butoxy, difluoromethoxy, trifluoromethoxy, cyclopropoxy, cyclobutoxy, methyl, ethyl, propyl, isopropyl, butyl, isobutyl, difluoromethyl, trifluoromethyl, cyclopropyl, cyclobutyl, isopropyl, tert-butyl, chloro, fluoro, 1-fluoropropan-2-yl, (S)-1-fluoropropan-2-yl, (R)-1-fluoropropan-2-yl, hydroxyethyl, 1-methoxy-2-methylpropan-2-yl, 1-methoxypropan-2-yl, (S)-1-methoxypropan-2-yl, (R)-1-methoxypropan-2-yl, 1-(methoxymethyl)cyclopropyl, 1-hydroxypropan-2-yl, oxetan-3-yl, tetrahydrofuran-3-yl, 1-methylcyclopropyl, deuteromethyl, deuteroethyl, deuteroisopropyl, deuteromethoxy, and deuteroethoxy.

17. The compound of any of claims 1-10, or a pharmaceutically acceptable salt or solvate thereof, wherein R¹ is independently selected from:

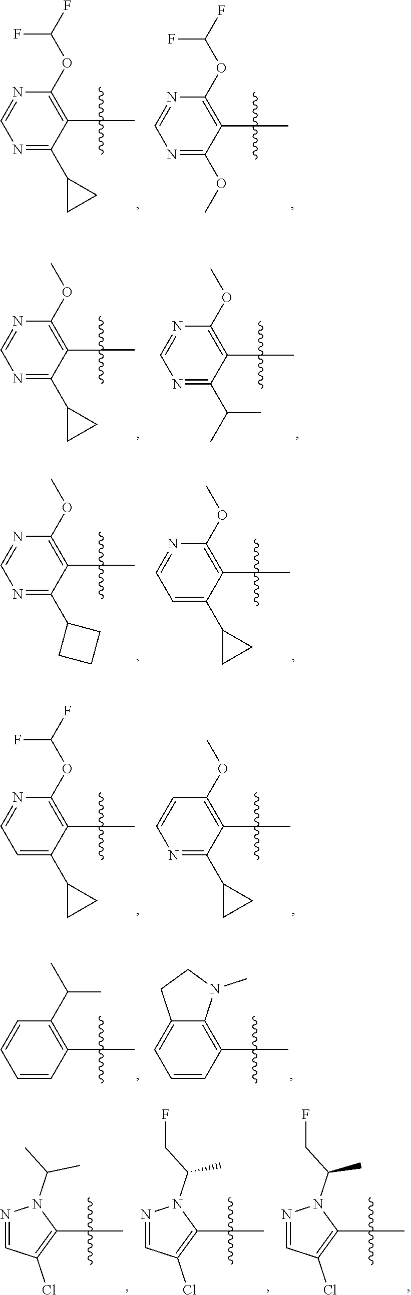

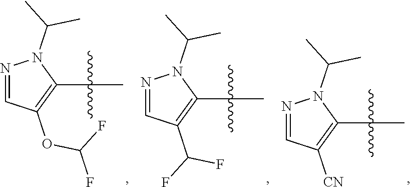

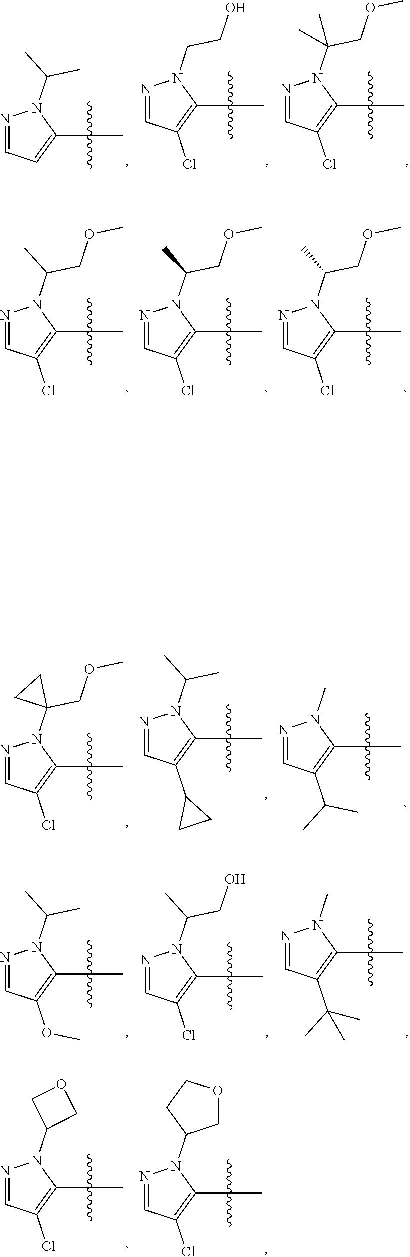

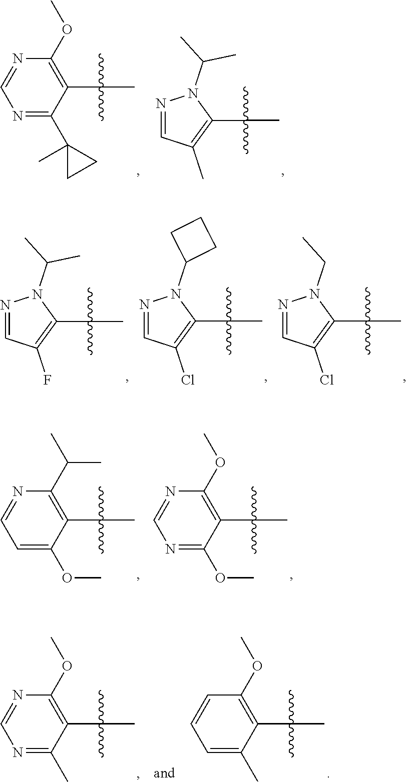

18. The compound of claim 1, or a pharmaceutically acceptable salt or solvate thereof, wherein the optional substituents on R² are independently selected from hydrogen, halo, nitro, cyano, hydroxy, amino, alkylamino, dialkylamino, haloalkyl, hydroxyalkyl, alkoxy, haloalkoxy, aryloxy, heteroaryloxy, optionally substituted cycloalkyloxy, aralkyl aralkyloxy, alkylthio, carboxamido, sulfonamido, alkylcarbonyl, arylcarbonyl, alkyl sulfonyl, aryl sulfonyl, carboxy, carboxyalkyl, alkyl, optionally substituted cycloalkyl, alkenyl, alkynyl, optionally substituted aryl, optionally substituted heteroaryl, optionally substituted heterocyclo, alkoxyalkyl, (amino)alkyl, hydroxyalkylamino, (alkylamino)alkyl, (dialkylamino)alkyl, (cyano)alkyl, (carboxamido)alkyl, mercaptoalkyl, (heterocyclo)alkyl, (cycloalkylamino)alkyl, (C₁₋₄haloalkoxy)alkyl, (heteroaryl)alkyl, and sulfonamido,
or
wherein two of the optional substituents on R² are taken together with the carbon or nitrogen atoms to which they are attached to form an optionally substituted cycloalkyl, optionally substituted heterocyclo, optionally substituted aryl, or optionally substituted heteroaryl group.

19. The compound of any of claims 1-9 and 18, or a pharmaceutically acceptable salt or solvate thereof, wherein R² is an optionally substituted 5- or 6-membered nitrogen-containing heteroaryl, wherein nitrogen is the only heteroatom.

20. The compound of any one of claims 1-9, 18, and 19, or a pharmaceutically acceptable salt or solvate thereof, wherein R² is an optionally substituted imidazolyl.

21. The compound of any one of claims 1-9, 18, and 19, or a pharmaceutically acceptable salt or solvate thereof, wherein R² is an optionally substituted pyridyl.

22. The compound of any one of claims 1-9, 18, and 19, or a pharmaceutically acceptable salt or solvate thereof, wherein R² is an optionally substituted pyrazolyl.

23. The compound of any one of claims 1-9, 18, and 19, or a pharmaceutically acceptable salt or solvate thereof, wherein R² is an optionally substituted pyridazinyl.

24. The compound of any one of claims 1-9, 18, and 19, or a pharmaceutically acceptable salt or solvate thereof, wherein R² is an optionally substituted pyrimidinyl.

25. The compound of any one of claims 1-9, 18, and 19, or a pharmaceutically acceptable salt or solvate thereof, wherein R² is an optionally substituted triazinyl.

26. The compound of any one of claims 1-9, 18, and 19, or a pharmaceutically acceptable salt or solvate thereof, wherein R² is an optionally substituted pyrazinyl.

27. The compound of any one of claims 1-9, 18, and 19, or a pharmaceutically acceptable salt or solvate thereof, wherein R² is an optionally substituted triazolyl.

28. The compound of any one of claims 1-9, 18, and 19, wherein R² is substituted and the substituents are independently selected from hydrogen, cyano, nitro, fluoro, chloro, bromo, methyl, ethyl, propyl, isopropyl, butyl, isobutyl, tert-butyl, trifluoromethyl, methoxy, ethoxy, isopropoxy, tert-butoxy, difluoromethoxy, trifluoromethoxy, 1-fluoropropan-2-yl, 2-fluoroethyl, amino, methylamino, ethylamino, dimethylamino, 2,2-difluoroethoxy, cyclopropoxy, morpholino, methoxymethyl, N,N-dimethylsulfonamido, cyclopropyl, methylaminomethyl, deuteromethyl, deuteroethyl, deuteroisopropyl, deuteromethoxy, and deuteroethoxy.

29. The compound of any one of claims 1-9, 18, and 19, or a pharmaceutically acceptable salt or solvate thereof, wherein R² is independently selected from:

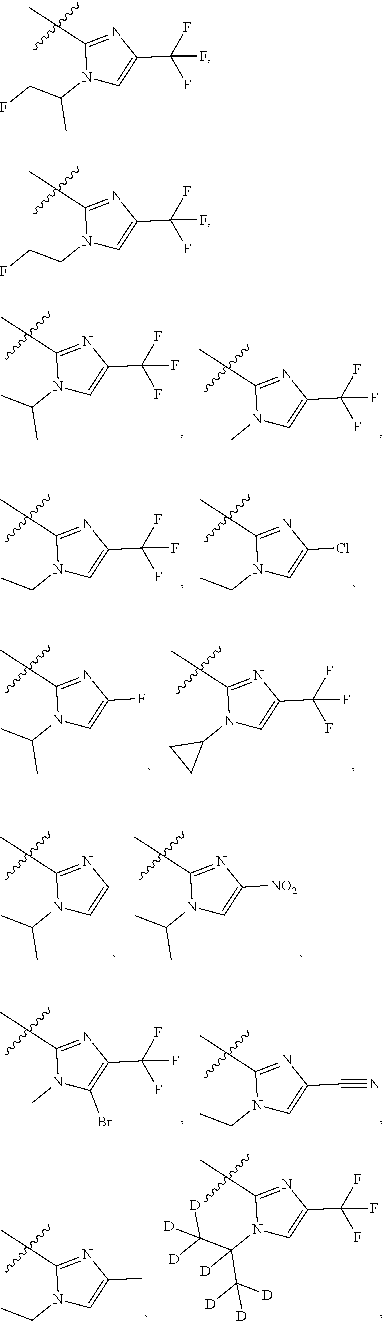

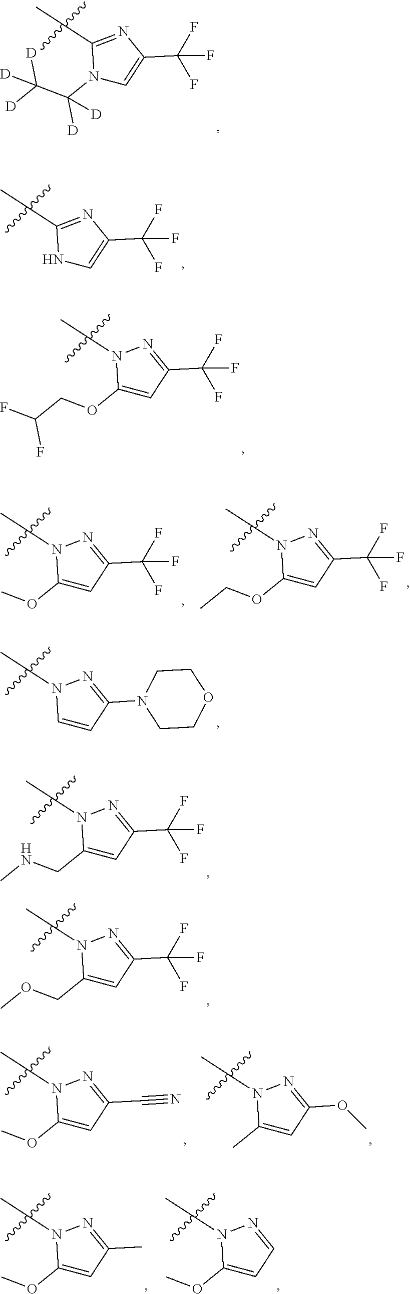

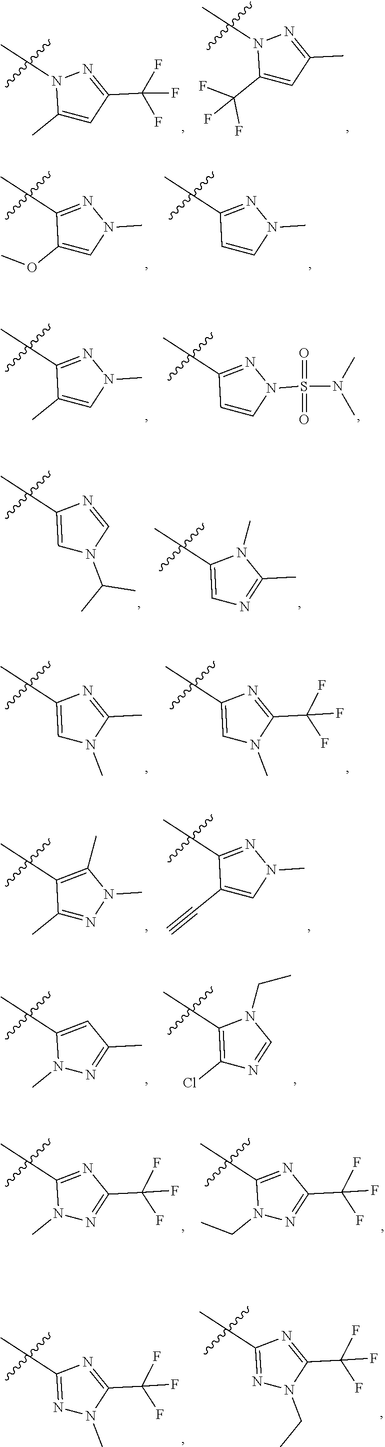

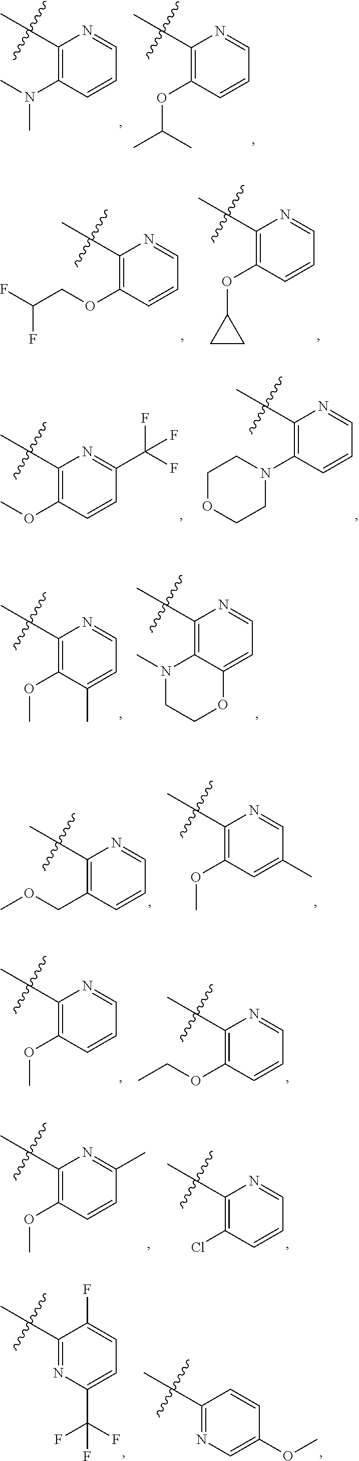

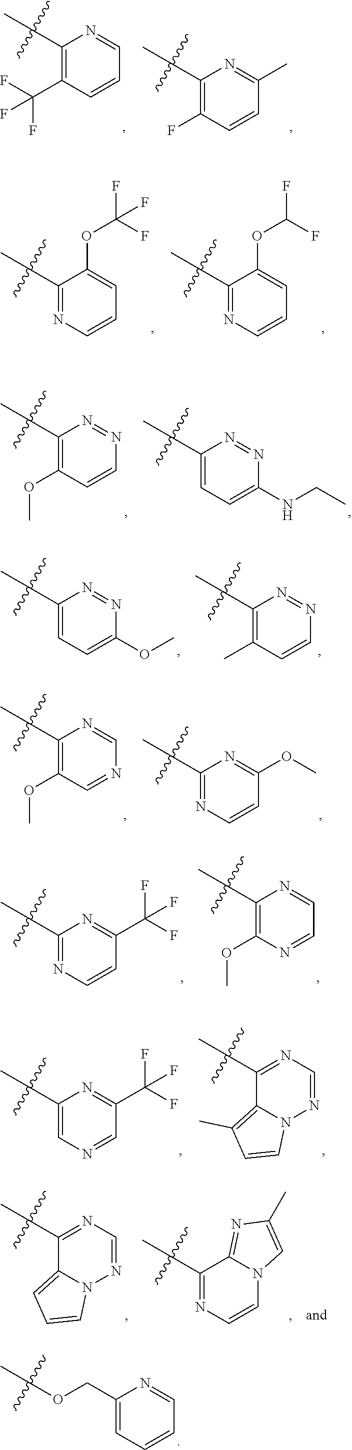

30. The compound of claim 1, or a pharmaceutically acceptable salt or solvate thereof, wherein 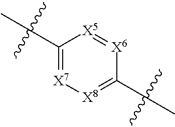
is independently selected from

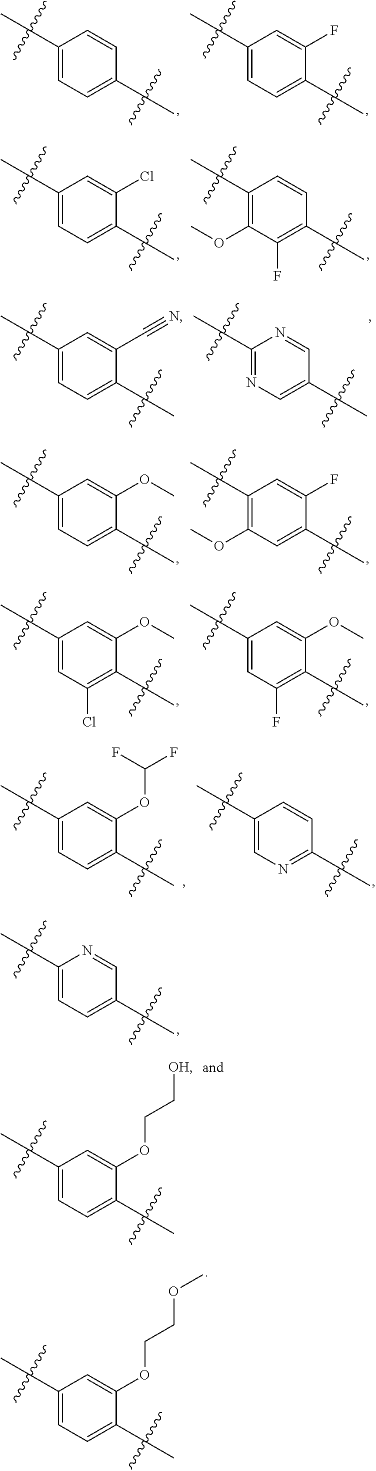

31. The compound of claim 1, or a pharmaceutically acceptable salt or solvate thereof, having Formula II, Formula III, Formula IV, Formula V, Formula VI, or Formula VII: 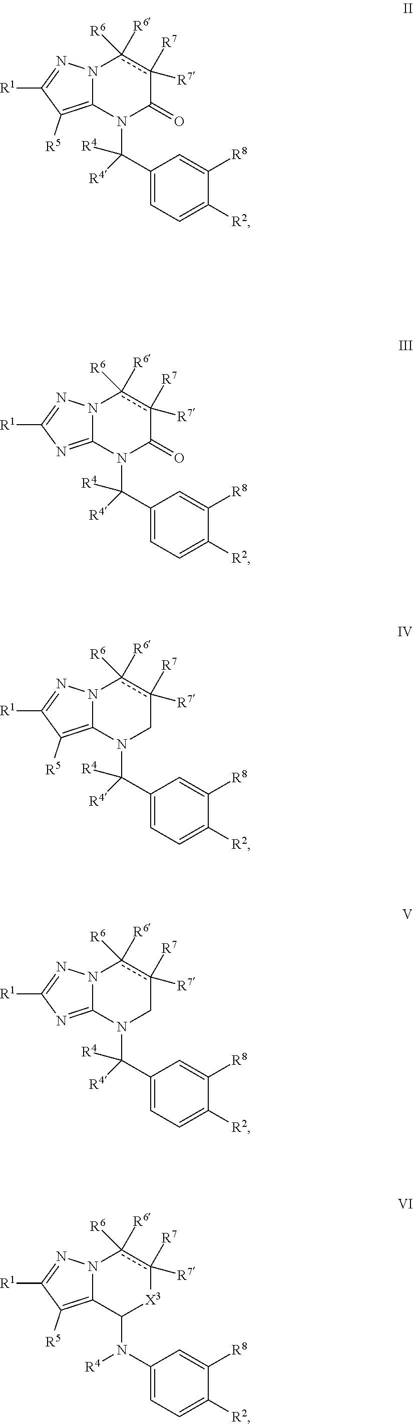 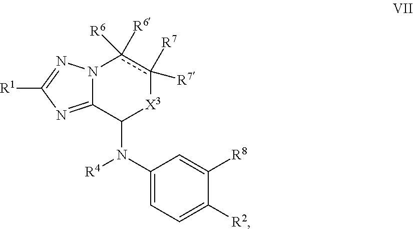
wherein:
R¹ is selected from 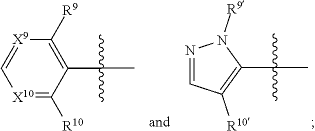
R² is selected from 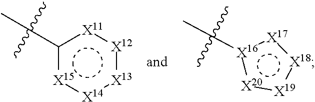
R⁴ and R^{4′} are independently selected from hydrogen, halo, and (C₁-C₃) alkyl; and
R⁵ is selected from hydrogen, halo, cyano, amino, alkylamino, dialkylamino, and (C₁-C₃) alkyl;
X⁹ is selected from N and CH;
X¹⁰ is selected from N and CH;
X¹¹ is selected from N and CR″;
X¹² is selected from N and CR¹²;
X¹³ is selected from N and CR¹³;
X¹⁴ is selected from N and CR¹⁴;
X¹⁵ is selected from N and CR¹⁵;
X¹⁶ is selected from N and CR¹⁶;
X¹⁷ is selected from N and CR¹⁷;
X¹⁸ is selected from N and CR¹⁸;
X¹⁹ is selected from N and CR¹⁹;
X²⁰ is selected from N and CR²⁰;
each of R⁹, R^{9′}, R¹⁰, R^{10′}, R¹¹, R¹², R¹³, R¹⁴, R¹⁵, R¹⁶, R¹⁷, R¹⁸, R¹⁹, R²⁰ is independently selected from the group consisting of hydrogen, halo, nitro, cyano, hydroxy, amino, alkylamino, dialkylamino, haloalkyl, hydroxyalkyl, alkoxy, haloalkoxy, aryloxy, heteroaryloxy, aralkyl aralkyloxy, alkylthio, carboxamido, sulfonamido, alkylcarbonyl, arylcarbonyl, alkyl sulfonyl, arylsulfonyl, carboxy, carboxyalkyl, alkyl, optionally substituted cycloalkyl, alkenyl, alkynyl, optionally substituted aryl, optionally substituted heteroaryl, optionally substituted heterocyclo, alkoxyalkyl, (amino)alkyl, hydroxyalkylamino, (alkylamino)alkyl, (dialkylamino)alkyl, (cyano)alkyl, (carboxamido)alkyl, mercaptoalkyl, (heterocyclo)alkyl, (cycloalkylamino)alkyl, (C₁₋₄ haloalkoxy)alkyl, (heteroaryl)alkyl, or sulfonamide; or wherein two of R¹¹, R¹², R¹³, R¹⁴, and R¹⁵ or two of R¹⁶, R¹⁷, R¹⁸, R¹⁹, and R²⁰ are taken together with the atom to which they are attached to form an optionally substituted cycloalkyl, optionally substituted heterocyclo, optionally substituted heteroaryl, or optionally substituted aryl.

32. The compound of claim 31, or a pharmaceutically acceptable salt or solvate thereof, having Formula VIII, or Formula IX 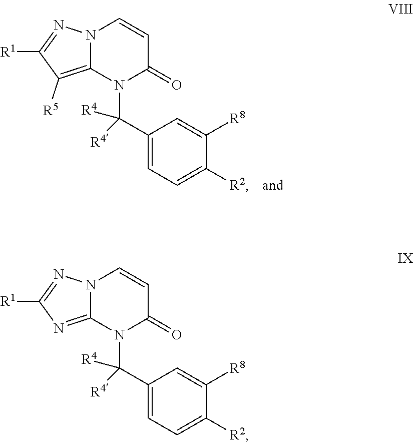
wherein:
R⁴ and R^{4′} are independently selected from hydrogen and methyl; and
R⁸ is selected from hydrogen and fluoro.

33. The compound of claim 31, or a pharmaceutically acceptable salt or solvate thereof, having Formula X, or Formula XI 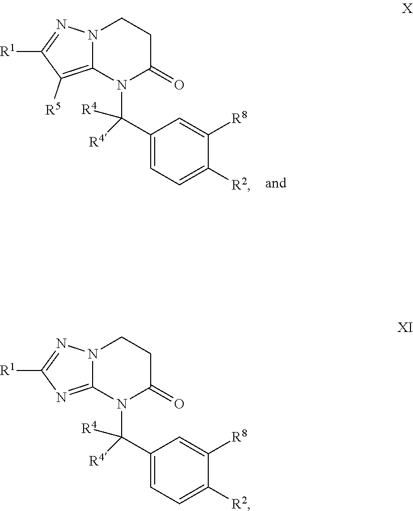
wherein:
R⁴ and R^{4′} are independently selected from hydrogen and methyl; and
R⁸ is selected from hydrogen and fluoro.

34. The compound of claim 31, or a pharmaceutically acceptable salt or solvate thereof, having Formula XII, or Formula XIII 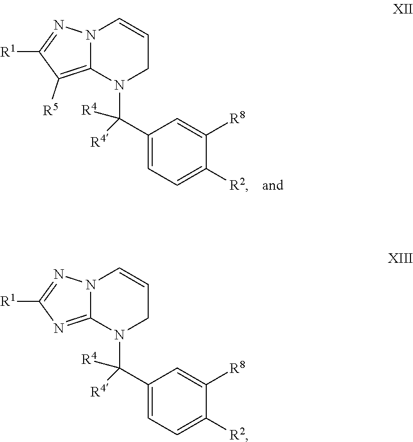
wherein:
R⁴ and R^{4′} are independently selected from hydrogen and methyl; and
R⁸ is selected from hydrogen and fluoro.

35. The compound of claim 31, or a pharmaceutically acceptable salt or solvate thereof, having Formula XIV, or Formula XV 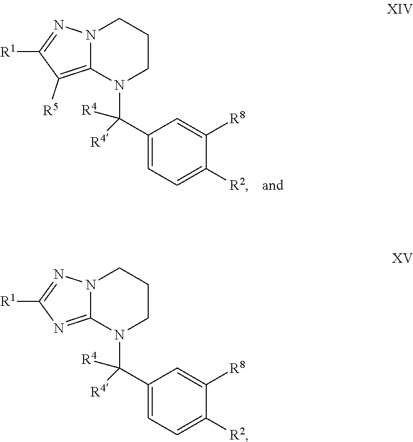
wherein:
R⁴ and R^{4′} are independently selected from hydrogen and methyl; and
R⁸ is selected from hydrogen and fluoro.

36. The compound of claim 1 selected from the group consisting of:
2-(4-cyclopropyl-6-methoxypyrimidin-5-yl)-4-(4-(1-methyl-4-(trifluoromethyl)-1H-imidazol-2-yl)benzyl)-6,7-dihydropyrazolo[1,5-α]pyrimidin-5(4H)-one;
2-(4-cyclopropyl-6-methoxypyrimidin-5-yl)-4-(3-fluoro-4-(1-methyl-4-(trifluoromethyl)-1H-imidazol-2-yl)benzyl)-6,7-dihydropyrazolo[1,5-α]pyrimidin-5(4H)-one;
2-(4-cyclopropyl-6-methoxypyrimidin-5-yl)-4-(1-(4-(1-methyl-4-(trifluoromethyl)-1H-imidazol-2-yl)phenyl)ethyl)-6,7-dihydropyrazolo[1,5-α]pyrimidin-5(4H)-one;
2-(4-cyclopropyl-6-methoxypyrimidin-5-yl)-4-(4-(5-methyl-3-(trifluoromethyl)-1H-pyrazol-1-yl)benzyl)-6,7-dihydropyrazolo[1,5-α]pyrimidin-5(4H)-one;
2-(4,6-dimethoxypyrimidin-5-yl)-4-(4-(1-ethyl-4-(trifluoromethyl)-1H-imidazol-2-yl)benzyl)-6,7-dihydropyrazolo[1,5-α]pyrimidin-5(4H)-one;
2-(1-isopropyl-4-methyl-1H-pyrazol-5-yl)-4-(4-(1-methyl-4-(trifluoromethyl)-1H-imidazol-2-yl)benzyl)-6,7-dihydropyrazolo[1,5-α]pyrimidin-5(4H)-one;
2-(4-cyclopropyl-6-methoxypyrimidin-5-yl)-4-(3-fluoro-4-(5-methoxy-3-(trifluoromethyl)-1H-pyrazol-1-yl)benzyl)-6,7-dihydropyrazolo[1,5-α]pyrimidin-5(4H)-one;
1-(4-((2-(4-cyclopropyl-6-methoxypyrimidin-5-yl)-5-oxo-6,7-dihydropyrazolo[1,5-α]pyrimidin-4(5H)-yl)methyl)-2-fluorophenyl)-5-methoxy-1H-pyrazole-3-carbonitrile;
4-(4-(1-ethyl-4-(trifluoromethyl)-1H-imidazol-2-yl)benzyl)-2-(1-isopropyl-4-methyl-1H-pyrazol-5-yl)-6,7-dihydropyrazolo[1,5-α]pyrimidin-5(4H)-one;
2-(1-isopropyl-4-methyl-1H-pyrazol-5-yl)-4-(4-(5-methyl-3-(trifluoromethyl)-1H-pyrazol-1-yl)benzyl)-6,7-dihydropyrazolo[1,5-α]pyrimidin-5(4H)-one;
2-(1-isopropyl-4-methyl-1H-pyrazol-5-yl)-4-(4-(1-methyl-3-(trifluoromethyl)-1H-1,2,4-triazol-5-yl)benzyl)-6,7-dihydropyrazolo[1,5-α]pyrimidin-5(4H)-one;
4-(3-fluoro-4-(5-methoxy-3-(trifluoromethyl)-1H-pyrazol-1-yl)benzyl)-2-(1-isopropyl-4-methyl-1H-pyrazol-5-yl)-6,7-dihydropyrazolo[1,5-α]pyrimidin-5(4H)-one;
2-(2-isopropyl-4-methoxypyridin-3-yl)-4-(4-(1-methyl-4-(trifluoromethyl)-1H-imidazol-2-yl)benzyl)-6,7-dihydropyrazolo[1,5-α]pyrimidin-5(4H)-one;
2-(4-chloro-1-isopropyl-1H-pyrazol-5-yl)-4-(3-fluoro-4-(5-methoxy-3-(trifluoromethyl)-1H-pyrazol-1-yl)benzyl)-6,7-dihydropyrazolo[1,5-α]pyrimidin-5(4H)-one;
4-(4-(1-ethyl-5-(trifluoromethyl)-1H-1,2,4-triazol-3-yl)benzyl)-2-(1-isopropyl-4-methyl-1H-pyrazol-5-yl)-6,7-dihydropyrazolo[1,5-α]pyrimidin-5(4H)-one;
2-(4-chloro-1-isopropyl-1H-pyrazol-5-yl)-4-(3-fluoro-4-(1-methyl-4-(trifluoromethyl)-1H-imidazol-2-yl)benzyl)-6,7-dihydropyrazolo[1,5-α]pyrimidin-5(4H)-one;
2-(4-chloro-1-ethyl-1H-pyrazol-5-yl)-4-(3-fluoro-4-(1-methyl-4-(trifluoromethyl)-1H-imidazol-2-yl)benzyl)-6,7-dihydropyrazolo[1,5-α]pyrimidin-5(4H)-one;
2-(4-cyclopropyl-6-methoxypyrimidin-5-yl)-4-(3-fluoro-4-(1-isopropyl-4-(trifluoromethyl)-1H-imidazol-2-yl)benzyl)-6,7-dihydropyrazolo[1,5-α]pyrimidin-5(4H)-one;
2-(4-chloro-1-cyclobutyl-1H-pyrazol-5-yl)-4-(3-fluoro-4-(1-methyl-4-(trifluoromethyl)-1H-imidazol-2-yl)benzyl)-6,7-dihydropyrazolo[1,5-α]pyrimidin-5(4H)-one;
2-(4-chloro-1-isopropyl-1H-pyrazol-5-yl)-4-(3-fluoro-4-(1-isopropyl-4-(trifluoromethyl)-1H-imidazol-2-yl)benzyl)-6,7-dihydropyrazolo[1,5-α]pyrimidin-5(4H)-one;
2-(4-fluoro-1-isopropyl-1H-pyrazol-5-yl)-4-(3-fluoro-4-(1-methyl-4-(trifluoromethyl)-1H-imidazol-2-yl)benzyl)-6,7-dihydropyrazolo[1,5-α]pyrimidin-5(4H)-one;
2-(4-chloro-1-isopropyl-1H-pyrazol-5-yl)-4-(4-(1-ethyl-3-(trifluoromethyl)-1H-1,2,4-triazol-5-yl)benzyl)-6,7-dihydropyrazolo[1,5-α]pyrimidin-5(4H)-one;
4-(4-(1-ethyl-4-(trifluoromethyl)-1H-imidazol-2-yl)benzyl)-2-(1-methylindolin-7-yl)-6,7-dihydropyrazolo[1,5-α]pyrimidin-5(4H)-one;
2-(4-cyclopropyl-6-methoxypyrimidin-5-yl)-4-(4-(1-methyl-4-(trifluoromethyl)-1H-imidazol-2-yl)benzyl)-4,5,6,7-tetrahydropyrazolo[1,5-α]pyrimidine;
2-(4-methoxy-6-methylpyrimidin-5-yl)-4-(4-(1-methyl-4-(trifluoromethyl)-1H-imidazol-2-yl)benzyl)-4,5,6,7-tetrahydropyrazolo[1,5-α]pyrimidine;
2-(4-methoxy-6-methylpyrimidin-5-yl)-4-(4-(5-methyl-3-(trifluoromethyl)-1H-pyrazol-1-yl)benzyl)-4,5,6,7-tetrahydropyrazolo[1,5-α]pyrimidine;
2-(4-cyclopropyl-6-methoxypyrimidin-5-yl)-4-(4-(5-methyl-3-(trifluoromethyl)-1H-pyrazol-1-yl)benzyl)-4,5,6,7-tetrahydropyrazolo[1,5-α]pyrimidine;
2-(4-cyclopropyl-6-methoxypyrimidin-5-yl)-4-(4-(1-ethyl-4-(trifluoromethyl)-1H-imidazol-2-yl)benzyl)-4,5,6,7-tetrahydropyrazolo[1,5-α]pyrimidine;
2-(4-chloro-1-isopropyl-1H-pyrazol-5-yl)-4-(4-(1-ethyl-4-(trifluoromethyl)-1H-imidazol-2-yl)-3-fluorobenzyl)-6-methyl-4,5,6,7-tetrahydropyrazolo[1,5-α]pyrimidine;
2′-(4-cyclopropyl-6-methoxypyrimidin-5-yl)-4′-(4-(1-ethyl-4-(trifluoromethyl)-1H-imidazol-2-yl)-3-fluorobenzyl)-4′,5′-dihydro-7′H-spiro[cyclopropane-1,6′-pyrazolo[1,5-α]pyrimidine];
2-(1-isopropyl-4-methyl-1H-pyrazol-5-yl)-6-methyl-4-(4-(1-methyl-4-(trifluoromethyl)-1H-imidazol-2-yl)benzyl)-6,7-dihydropyrazolo[1,5-α]pyrimidin-5(4H)-one;
2-(4-cyclopropyl-6-methoxypyrimidin-5-yl)-6-methyl-4-(4-(1-methyl-4-(trifluoromethyl)-1H-imidazol-2-yl)benzyl)-6,7-dihydropyrazolo[1,5-α]pyrimidin-5(4H)-one;
3-chloro-2-(4-cyclopropyl-6-methoxypyrimidin-5-yl)-4-(4-(1-methyl-4-(trifluoromethyl)-1H-imidazol-2-yl)benzyl)-6,7-dihydropyrazolo[1,5-α]pyrimidin-5(4H)-one;
3-chloro-2-(1-isopropyl-4-methyl-1H-pyrazol-5-yl)-4-(4-(1-methyl-4-(trifluoromethyl)-1H-imidazol-2-yl)benzyl)-6,7-dihydropyrazolo[1,5-α]pyrimidin-5(4H)-one;
2-(4-cyclopropyl-6-methoxypyrimidin-5-yl)-3-methyl-4-(4-(1-methyl-4-(trifluoromethyl)-1H-imidazol-2-yl)benzyl)-6,7-dihydropyrazolo[1,5-α]pyrimidin-5(4H)-one;
2-(1-isopropyl-4-methyl-1H-pyrazol-5-yl)-7-methyl-4-(4-(1-methyl-4-(trifluoromethyl)-1H-imidazol-2-yl)benzyl)-6,7-dihydropyrazolo[1,5-α]pyrimidin-5(4H)-one;
2-(1-isopropyl-4-methyl-1H-pyrazol-5-yl)-4-(4-(1-methyl-4-(trifluoromethyl)-1H-imidazol-2-yl)benzyl)-6,7-dihydro-[1,2,4]triazolo[1,5-α]pyrimidin-5(4H)-one;
4-(4-(1-isopropyl-4-(trifluoromethyl)-1H-imidazol-2-yl)benzyl)-2-(1-isopropyl-4-methyl-1H-pyrazol-5-yl)-6,7-dihydro-[1,2,4]triazolo[1,5-α]pyrimidin-5(4H)-one;
2-(4-chloro-1-isopropyl-1H-pyrazol-5-yl)-4-(4-(1-methyl-4-(trifluoromethyl)-1H-imidazol-2-yl)benzyl)-6,7-dihydro-[1,2,4]triazolo[1,5-α]pyrimidin-5(4H)-one;
2-(4-chloro-1-isopropyl-1H-pyrazol-5-yl)-4-(3-fluoro-4-(1-isopropyl-4-(trifluoromethyl)-1H-imidazol-2-yl)benzyl)-6,7-dihydro-[1,2,4]triazolo[1,5-α]pyrimidin-5(4H)-one;
2-(4-chloro-1-isopropyl-1H-pyrazol-5-yl)-4-(4-(1-isopropyl-4-(trifluoromethyl)-1H-imidazol-2-yl)benzyl)-6,7-dihydro-[1,2,4]triazolo[1,5-α]pyrimidin-5(4H)-one;
2-(4-chloro-1-isopropyl-1H-pyrazol-5-yl)-4-(4-(1-ethyl-4-(trifluoromethyl)-1H-imidazol-2-yl)-3-fluorobenzyl)-6,7-dihydro-[1,2,4]triazolo[1,5-α]pyrimidin-5(4H)-one;
2-(4-cyclopropyl-6-methoxypyrimidin-5-yl)-4-(4-(1-methyl-4-(trifluoromethyl)-1H-imidazol-2-yl)benzyl)-6,7-dihydro-[1,2,4]triazolo[1,5-α]pyrimidin-5(4H)-one;
2-(4-cyclopropyl-6-methoxypyrimidin-5-yl)-4-(4-(1-ethyl-4-(trifluoromethyl)-1H-imidazol-2-yl)-3-fluorobenzyl)-6,7-dihydro-[1,2,4]triazolo[1,5-α]pyrimidin-5(4H)-one;
4-(4-(1-ethyl-4-(trifluoromethyl)-1H-imidazol-2-yl)benzyl)-2-(1-isopropyl-4-methoxy-1H-pyrazol-5-yl)-6,7-dihydro-[1,2,4]triazolo[1,5-α]pyrimidin-5(4H)-one;
4-(4-(1-isopropyl-4-(trifluoromethyl)-1H-imidazol-2-yl)-3-methoxybenzyl)-2-(4-isopropyl-6-methoxypyrimidin-5-yl)-6,7-dihydro-[1,2,4]triazolo[1,5-α]pyrimidin-5(4H)-one;
2-(1-isopropyl-4-methyl-1H-pyrazol-5-yl)-4-(4-(1-methyl-4-(trifluoromethyl)-1H-imidazol-2-yl)benzyl)-4,5,6,7-tetrahydropyrazolo[1,5-α]pyrimidine;
2-(4-cyclopropyl-6-methoxypyrimidin-5-yl)-5-methyl-4-(4-(1-methyl-4-(trifluoromethyl)-1H-imidazol-2-yl)benzyl)-4,5,6,7-tetrahydropyrazolo[1,5-α]pyrimidine;
2-(4-cyclopropyl-6-methoxypyrimidin-5-yl)-4-(4-(1-ethyl-4-(trifluoromethyl)-1H-imidazol-2-yl)-3-fluorobenzyl)-5-methyl-4,5,6,7-tetrahydropyrazolo[1,5-α]pyrimidine;
2-(4-chloro-1-isopropyl-1H-pyrazol-5-yl)-4-(3-fluoro-4-(1-isopropyl-4-(trifluoromethyl)-1H-imidazol-2-yl)benzyl)-5-methyl-4,5,6,7-tetrahydropyrazolo[1,5-α]pyrimidine;
2-(4-cyclopropyl-6-methoxypyrimidin-5-yl)-7-methyl-4-(4-(1-methyl-4-(trifluoromethyl)-1H-imidazol-2-yl)benzyl)-4,5,6,7-tetrahydropyrazolo[1,5-α]pyrimidine;
2-(1-isopropyl-4-methyl-1H-pyrazol-5-yl)-4-(4-(5-methyl-3-(trifluoromethyl)-1H-pyrazol-1-yl)benzyl)-4,5,6,7-tetrahydropyrazolo[1,5-α]pyrimidine;
2-(1-isopropyl-4-methyl-1H-pyrazol-5-yl)-4-(4-(1-methyl-3-(trifluoromethyl)-1H-1,2,4-triazol-5-yl)benzyl)-4,5,6,7-tetrahydropyrazolo[1,5-α]pyrimidine;
4-(4-(1-isopropyl-4-(trifluoromethyl)-1H-imidazol-2-yl)benzyl)-2-(1-isopropyl-4-methyl-1H-pyrazol-5-yl)-4,5,6,7-tetrahydropyrazolo[1,5-α]pyrimidine;
4-(3-fluoro-4-(1-methyl-4-(trifluoromethyl)-1H-imidazol-2-yl)benzyl)-2-(1-isopropyl-4-methyl-1H-pyrazol-5-yl)-4,5,6,7-tetrahydropyrazolo[1,5-α]pyrimidine;
4-(3-fluoro-4-(5-methyl-3-(trifluoromethyl)-1H-pyrazol-1-yl)benzyl)-2-(1-isopropyl-4-methyl-1H-pyrazol-5-yl)-4,5,6,7-tetrahydropyrazolo[1,5-α]pyrimidine;
4-(3-fluoro-4-(5-methoxy-3-(trifluoromethyl)-1H-pyrazol-1-yl)benzyl)-2-(1-isopropyl-4-methyl-1H-pyrazol-5-yl)-4,5,6,7-tetrahydropyrazolo[1,5-α]pyrimidine;
2-(4-cyclopropyl-6-methoxypyrimidin-5-yl)-4-(4-(1-isopropyl-4-(trifluoromethyl)-1H-imidazol-2-yl)benzyl)-4,5,6,7-tetrahydropyrazolo[1,5-α]pyrimidine;
2-(4-chloro-1-isopropyl-1H-pyrazol-5-yl)-4-(3-fluoro-4-(1-isopropyl-4-(trifluoromethyl)-1H-imidazol-2-yl)benzyl)-4,5,6,7-tetrahydropyrazolo[1,5-α]pyrimidine;
2-(4-chloro-1-isopropyl-1H-pyrazol-5-yl)-4-(4-(1-ethyl-4-(trifluoromethyl)-1H-imidazol-2-yl)-3-fluorobenzyl)-4,5,6,7-tetrahydropyrazolo[1,5-α]pyrimidine;
2-(4-chloro-1-isopropyl-1H-pyrazol-5-yl)-4-(3-fluoro-4-(1-methyl-4-(trifluoromethyl)-1H-imidazol-2-yl)benzyl)-4,5,6,7-tetrahydropyrazolo[1,5-α]pyrimidine;
2-(4-chloro-1-isopropyl-1H-pyrazol-5-yl)-4-(4-(1-(2-fluoroethyl)-4-(trifluoromethyl)-1H-imidazol-2-yl)benzyl)-4,5,6,7-tetrahydropyrazolo[1,5-α]pyrimidine;
2-(4-cyclopropyl-6-methoxypyrimidin-5-yl)-4-(3-fluoro-4-(1-isopropyl-4-(trifluoromethyl)-1H-imidazol-2-yl)benzyl)-4,5,6,7-tetrahydropyrazolo[1,5-α]pyrimidine;
2-(4-cyclopropyl-6-methoxypyrimidin-5-yl)-4-(4-(1-ethyl-4-(trifluoromethyl)-1H-imidazol-2-yl)-3-fluorobenzyl)-4,5,6,7-tetrahydropyrazolo[1,5-α]pyrimidine;
2-(4-chloro-1-isopropyl-1H-pyrazol-5-yl)-4-(1-(4-(1-ethyl-4-(trifluoromethyl)-1H-imidazol-2-yl)phenyl)ethyl)-4,5,6,7-tetrahydropyrazolo[1,5-α]pyrimidine;
2-(4-cyclopropyl-6-methoxypyrimidin-5-yl)-4-(1-(4-(1-ethyl-4-(trifluoromethyl)-1H-imidazol-2-yl)phenyl)ethyl)-4,5,6,7-tetrahydropyrazolo[1,5-α]pyrimidine;
(S)-2-(4-chloro-1-isopropyl-1H-pyrazol-5-yl)-4-(1-(4-(1-ethyl-4-(trifluoromethyl)-1H-imidazol-2-yl)phenyl)ethyl)-4,5,6,7-tetrahydropyrazolo[1,5-α]pyrimidine;
(R)-2-(4-chloro-1-isopropyl-1H-pyrazol-5-yl)-4-(1-(4-(1-ethyl-4-(trifluoromethyl)-1H-imidazol-2-yl)phenyl)ethyl)-4,5,6,7-tetrahydropyrazolo[1,5-α]pyrimidine;
2-(4-cyclopropyl-6-methoxypyrimidin-5-yl)-4-(4-(4-(trifluoromethyl)-1H-imidazol-2-yl)benzyl)-6,7-dihydropyrazolo[1,5-α]pyrimidin-5(4H)-one;
2-(1-isopropyl-4-methyl-1H-pyrazol-5-yl)-4-(4-(4-(trifluoromethyl)-1H-imidazol-2-yl)benzyl)-6,7-dihydropyrazolo[1,5-α]pyrimidin-5(4H)-one;
2-(4-cyclopropyl-6-methoxypyrimidin-5-yl)-4-(4-(1-ethyl-4-(trifluoromethyl)-1H-imidazol-2-yl)benzyl)-6,7-dihydropyrazolo[1,5-α]pyrimidin-5(4H)-one;
2-(4-cyclopropyl-6-methoxypyrimidin-5-yl)-4-(4-(1-isopropyl-4-(trifluoromethyl)-1H-imidazol-2-yl)benzyl)-6,7-dihydropyrazolo[1,5-α]pyrimidin-5(4H)-one;
4-(4-(1-isopropyl-4-(trifluoromethyl)-1H-imidazol-2-yl)benzyl)-2-(1-isopropyl-4-methyl-1H-pyrazol-5-yl)-6,7-dihydropyrazolo[1,5-α]pyrimidin-5(4H)-one;
2-(2-methoxy-6-methylphenyl)-4-(4-(1-methyl-4-(trifluoromethyl)-1H-imidazol-2-yl)benzyl)-4,5,6,7-tetrahydropyrazolo[1,5-α]pyrimidine;
2-(2-methoxy-6-methylphenyl)-4-(4-(1-methyl-4-(trifluoromethyl)-1H-imidazol-2-yl)benzyl)-6,7-dihydropyrazolo[1,5-α]pyrimidin-5(4H)-one;
2-(4-cyclopropyl-6-methoxypyrimidin-5-yl)-6,6-dimethyl-4-(4-(1-methyl-4-(trifluoromethyl)-1H-imidazol-2-yl)benzyl)-6,7-dihydropyrazolo[1,5-α]pyrimidin-5(4H)-one;
2-(4-chloro-1-isopropyl-1H-pyrazol-5-yl)-4-(4-(1-ethyl-4-(trifluoromethyl)-1H-imidazol-2-yl)-3-fluorobenzyl)-6,7-dihydropyrazolo[1,5-α]pyrimidin-5(4H)-one;
2-(4-cyclopropyl-6-methoxypyrimidin-5-yl)-4-(4-(1-ethyl-4-(trifluoromethyl)-1H-imidazol-2-yl)-3-fluorobenzyl)-6,7-dihydropyrazolo[1,5-α]pyrimidin-5(4H)-one;
2-(4-chloro-1-isopropyl-1H-pyrazol-5-yl)-4-(4-(1-(2-fluoroethyl)-4-(trifluoromethyl)-1H-imidazol-2-yl)benzyl)-6,7-dihydropyrazolo[1,5-α]pyrimidin-5(4H)-one;
2-(4-cyclopropyl-6-methoxypyrimidin-5-yl)-4-(4-(5-methoxy-3-(trifluoromethyl)-1H-pyrazol-1-yl)benzyl)-6,7-dihydropyrazolo[1,5-α]pyrimidin-5(4H)-one;
2-(4-cyclopropyl-6-methoxypyrimidin-5-yl)-4-(4-(1-(2-fluoroethyl)-4-(trifluoromethyl)-1H-imidazol-2-yl)benzyl)-6,7-dihydropyrazolo[1,5-α]pyrimidin-5(4H)-one;
2-(4-chloro-1-isopropyl-1H-pyrazol-5-yl)-4-((6-(1-methyl-4-(trifluoromethyl)-1H-imidazol-2-yl)pyridin-3-yl)methyl)-6,7-dihydropyrazolo[1,5-α]pyrimidin-5(4H)-one;
2-(4-cyclopropyl-6-methoxypyrimidin-5-yl)-4-((6-(1-methyl-4-(trifluoromethyl)-1H-imidazol-2-yl)pyridin-3-yl)methyl)-6,7-dihydropyrazolo[1,5-α]pyrimidin-5(4H)-one;
2-(4-chloro-1-isopropyl-1H-pyrazol-5-yl)-4-(4-(1-ethyl-3-(trifluoromethyl)-1H-1,2,4-triazol-5-yl)benzyl)-6,7-dihydropyrazolo[1,5-α]pyrimidin-5(4H)-one;
4-(4-(1-ethyl-4-(trifluoromethyl)-1H-imidazol-2-yl)benzyl)-2-(4-fluoro-1-isopropyl-1H-pyrazol-5-yl)-6,7-dihydropyrazolo[1,5-α]pyrimidin-5(4H)-one;
2-(4-chloro-1-isopropyl-1H-pyrazol-5-yl)-4-(4-(5-methyl-3-(trifluoromethyl)-1H-pyrazol-1-yl)benzyl)-6,7-dihydropyrazolo[1,5-α]pyrimidin-5(4H)-one;
2-(4-cyclopropyl-6-methoxypyrimidin-5-yl)-4-(4-(1-ethyl-3-(trifluoromethyl)-1H-1,2,4-triazol-5-yl)benzyl)-6,7-dihydropyrazolo[1,5-α]pyrimidin-5(4H)-one;
2-(4-cyclopropyl-6-methoxypyrimidin-5-yl)-4-(3-fluoro-4-(3-methyl-5-(trifluoromethyl)-1H-pyrazol-1-yl)benzyl)-6,7-dihydropyrazolo[1,5-α]pyrimidin-5(4H)-one;
2-(4-chloro-1-isopropyl-1H-pyrazol-5-yl)-4-((6-(5-methoxy-3-(trifluoromethyl)-1H-pyrazol-1-yl)pyridin-3-yl)methyl)-6,7-dihydropyrazolo[1,5-α]pyrimidin-5(4H)-one;
2-(4-cyclopropyl-6-methoxypyrimidin-5-yl)-4-(4-(1,4-dimethyl-1H-pyrazol-3-yl)benzyl)-6,7-dihydropyrazolo[1,5-α]pyrimidin-5(4H)-one;
2-(4-chloro-1-isopropyl-1H-pyrazol-5-yl)-4-(4-(1-isopropyl-4-(trifluoromethyl)-1H-imidazol-2-yl)benzyl)-6,7-dihydropyrazolo[1,5-α]pyrimidin-5(4H)-one;
2-(4-chloro-1-isopropyl-1H-pyrazol-5-yl)-4-(3-fluoro-4-(3-methyl-5-(trifluoromethyl)-1H-pyrazol-1-yl)benzyl)-6,7-dihydropyrazolo[1,5-α]pyrimidin-5(4H)-one;
2-(4-chloro-1-isopropyl-1H-pyrazol-5-yl)-4-(4-(1,4-dimethyl-1H-pyrazol-3-yl)benzyl)-6,7-dihydropyrazolo[1,5-α]pyrimidin-5(4H)-one;
2-(4-cyclopropyl-6-methoxypyrimidin-5-yl)-4-((6-(5-methoxy-3-(trifluoromethyl)-1H-pyrazol-1-yl)pyridin-3-yl)methyl)-6,7-dihydropyrazolo[1,5-α]pyrimidin-5(4H)-one;
2-(4-chloro-1-isopropyl-1H-pyrazol-5-yl)-4-(4-(1-methyl-1H-pyrazol-3-yl)benzyl)-6,7-dihydropyrazolo[1,5-α]pyrimidin-5(4H)-one;
2-(4-chloro-1-isopropyl-1H-pyrazol-5-yl)-4-(4-(5-methoxy-3-(trifluoromethyl)-1H-pyrazol-1-yl)benzyl)-6,7-dihydropyrazolo[1,5-α]pyrimidin-5(4H)-one;
2-(4-chloro-1-isopropyl-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one;
2-(4-chloro-1-isopropyl-1H-pyrazol-5-yl)-4-(4-(1-methyl-4-(trifluoromethyl)-1H-imidazol-2-yl)benzyl)-6,7-dihydropyrazolo[1,5-α]pyrimidin-5(4H)-one;
2-(4-cyclopropyl-6-methoxypyrimidin-5-yl)-4-(4-(5-(methoxymethyl)-3-(trifluoromethyl)-1H-pyrazol-1-yl)benzyl)-6,7-dihydropyrazolo[1,5-α]pyrimidin-5(4H)-one;
2-(4-chloro-1-isopropyl-1H-pyrazol-5-yl)-4-(4-(5-(methoxymethyl)-3-(trifluoromethyl)-1H-pyrazol-1-yl)benzyl)-6,7-dihydropyrazolo[1,5-α]pyrimidin-5(4H)-one;
2-(4-chloro-1-isopropyl-1H-pyrazol-5-yl)-4-(4-(1-methyl-5-(trifluoromethyl)-1H-1,2,4-triazol-3-yl)benzyl)-6,7-dihydropyrazolo[1,5-α]pyrimidin-5(4H)-one;
2-(4-chloro-1-isopropyl-1H-pyrazol-5-yl)-4-(4-(1-methyl-3-(trifluoromethyl)-1H-1,2,4-triazol-5-yl)benzyl)-6,7-dihydropyrazolo[1,5-α]pyrimidin-5(4H)-one;
2-(4-cyclopropyl-6-methoxypyrimidin-5-yl)-4-(3-fluoro-4-(5-methyl-3-(trifluoromethyl)-1H-pyrazol-1-yl)benzyl)-6,7-dihydropyrazolo[1,5-α]pyrimidin-5(4H)-one;
2-(4-chloro-1-isopropyl-1H-pyrazol-5-yl)-4-(3-fluoro-4-(5-methyl-3-(trifluoromethyl)-1H-pyrazol-1-yl)benzyl)-6,7-dihydropyrazolo[1,5-α]pyrimidin-5(4H)-one;
2-(4-cyclopropyl-6-methoxypyrimidin-5-yl)-4-((6-(1-ethyl-4-(trifluoromethyl)-1H-imidazol-2-yl)pyridin-3-yl)methyl)-6,7-dihydropyrazolo[1,5-α]pyrimidin-5(4H)-one;
1-(4-((2-(4-chloro-1-isopropyl-1H-pyrazol-5-yl)-5-oxo-6,7-dihydropyrazolo[1,5-α]pyrimidin-4(5H)-yl)methyl)phenyl)-5-methoxy-1H-pyrazole-3-carbonitrile;
1-(4-((2-(4-cyclopropyl-6-methoxypyrimidin-5-yl)-5-oxo-6,7-dihydropyrazolo[1,5-α]pyrimidin-4(5H)-yl)methyl)phenyl)-5-methoxy-1H-pyrazole-3-carbonitrile;
2-(4-cyclopropyl-6-methoxypyrimidin-5-yl)-4-(4-(1-isopropyl-4-(trifluoromethyl)-1H-imidazol-2-yl)-3-methoxybenzyl)-6,7-dihydropyrazolo[1,5-α]pyrimidin-5(4H)-one;
2-(4-chloro-1-isopropyl-1H-pyrazol-5-yl)-4-(4-(1-isopropyl-4-(trifluoromethyl)-1H-imidazol-2-yl)-3-methoxybenzyl)-6,7-dihydropyrazolo[1,5-α]pyrimidin-5(4H)-one;
2-(4-chloro-1-isopropyl-1H-pyrazol-5-yl)-4-(1-(4-fluorophenyl)ethyl)-6,7-dihydropyrazolo[1,5-α]pyrimidin-5(4H)-one;
2-(4-chloro-1-isopropyl-1H-pyrazol-5-yl)-4-((6-(1-ethyl-4-(trifluoromethyl)-1H-imidazol-2-yl)pyridin-3-yl)methyl)-6,7-dihydropyrazolo[1,5-α]pyrimidin-5(4H)-one;
2-(4-chloro-1-isopropyl-1H-pyrazol-5-yl)-4-(4-(1-isopropyl-4-nitro-1H-imidazol-2-yl)benzyl)-6,7-dihydropyrazolo[1,5-α]pyrimidin-5(4H)-one;
2-(4-chloro-1-isopropyl-1H-pyrazol-5-yl)-4-(1-(4-(1-ethyl-4-(trifluoromethyl)-1H-imidazol-2-yl)phenyl)ethyl)-6,7-dihydropyrazolo[1,5-α]pyrimidin-5(4H)-one;
2-(4-cyclopropyl-6-methoxypyrimidin-5-yl)-4-(1-(4-(1-ethyl-4-(trifluoromethyl)-1H-imidazol-2-yl)phenyl)ethyl)-6,7-dihydropyrazolo[1,5-α]pyrimidin-5(4H)-one;
2-(4-chloro-1-isopropyl-1H-pyrazol-5-yl)-4-(4-(5-ethoxy-3-(trifluoromethyl)-1H-pyrazol-1-yl)benzyl)-6,7-dihydropyrazolo[1,5-α]pyrimidin-5(4H)-one;
2-(4-chloro-1-isopropyl-1H-pyrazol-5-yl)-4-(4-(1-methyl-2-(trifluoromethyl)-1H-imidazol-4-yl)benzyl)-6,7-dihydropyrazolo[1,5-α]pyrimidin-5(4H)-one;
2-(4-chloro-1-isopropyl-1H-pyrazol-5-yl)-4-(4-(5-methoxy-1H-pyrazol-1-yl)benzyl)-6,7-dihydropyrazolo[1,5-α]pyrimidin-5(4H)-one; 4-(4-(4-chloro-1-ethyl-1H-imidazol-2-yl)benzyl)-2-(4-chloro-1-isopropyl-1H-pyrazol-5-yl)-6,7-dihydropyrazolo[1,5-α]pyrimidin-5(4H)-one;
4-(4-(4-chloro-1-ethyl-1H-imidazol-5-yl)benzyl)-2-(4-chloro-1-isopropyl-1H-pyrazol-5-yl)-6,7-dihydropyrazolo[1,5-α]pyrimidin-5(4H)-one;
2-(4-chloro-1-isopropyl-1H-pyrazol-5-yl)-4-(1-(4-(1-ethyl-4-(trifluoromethyl)-1H-imidazol-2-yl)-3-fluorophenyl)ethyl)-6,7-dihydropyrazolo[1,5-α]pyrimidin-5(4H)-one;
2-(4-cyclopropyl-6-methoxypyrimidin-5-yl)-7-methyl-4-(4-(1-methyl-4-(trifluoromethyl)-1H-imidazol-2-yl)benzyl)-6,7-dihydropyrazolo[1,5-α]pyrimidin-5(4H)-one;
2-(4-cyclopropyl-6-methoxypyrimidin-5-yl)-4-(1-(4-(1-ethyl-4-(trifluoromethyl)-1H-imidazol-2-yl)-3-fluorophenyl)ethyl)-6,7-dihydropyrazolo[1,5-α]pyrimidin-5(4H)-one;
2-(4-chloro-1-isopropyl-1H-pyrazol-5-yl)-4-(5-(1-ethyl-4-(trifluoromethyl)-1H-imidazol-2-yl)-6-fluoro-2,3-dihydro-1H-inden-1-yl)-6,7-dihydropyrazolo[1,5-α]pyrimidin-5(4H)-one;
(S)-2-(4-cyclopropyl-6-methoxypyrimidin-5-yl)-4-(1-(4-(1-isopropyl-4-(trifluoromethyl)-1H-imidazol-2-yl)phenyl)ethyl)-6,7-dihydropyrazolo[1,5-α]pyrimidin-5(4H)-one;
(R)-2-(4-cyclopropyl-6-methoxypyrimidin-5-yl)-4-(1-(4-(1-isopropyl-4-(trifluoromethyl)-1H-imidazol-2-yl)phenyl)ethyl)-6,7-dihydropyrazolo[1,5-α]pyrimidin-5(4H)-one;
(S)-2-(4-cyclopropyl-6-methoxypyrimidin-5-yl)-4-(1-(4-(1-ethyl-4-(trifluoromethyl)-1H-imidazol-2-yl)phenyl)ethyl)-6,7-dihydropyrazolo[1,5-α]pyrimidin-5(4H)-one;
(R)-2-(4-cyclopropyl-6-methoxypyrimidin-5-yl)-4-(1-(4-(1-ethyl-4-(trifluoromethyl)-1H-imidazol-2-yl)phenyl)ethyl)-6,7-dihydropyrazolo[1,5-α]pyrimidin-5(4H)-one;
(S)-2-(4-chloro-1-isopropyl-1H-pyrazol-5-yl)-4-(1-(4-(1-ethyl-4-(trifluoromethyl)-1H-imidazol-2-yl)phenyl)ethyl)-6,7-dihydropyrazolo[1,5-α]pyrimidin-5(4H)-one;
(R)-2-(4-chloro-1-isopropyl-1H-pyrazol-5-yl)-4-(1-(4-(1-ethyl-4-(trifluoromethyl)-1H-imidazol-2-yl)phenyl)ethyl)-6,7-dihydropyrazolo[1,5-α]pyrimidin-5(4H)-one;
(S)-2-(4-cyclopropyl-6-methoxypyrimidin-5-yl)-4-(1-(4-(1-isopropyl-4-(trifluoromethyl)-1H-imidazol-2-yl)-3-methoxyphenyl)ethyl)-6,7-dihydropyrazolo[1,5-α]pyrimidin-5(4H)-one;
(R)-2-(4-cyclopropyl-6-methoxypyrimidin-5-yl)-4-(1-(4-(1-isopropyl-4-(trifluoromethyl)-1H-imidazol-2-yl)-3-methoxyphenyl)ethyl)-6,7-dihydropyrazolo[1,5-α]pyrimidin-5(4H)-one;
2-(4-chloro-1-isopropyl-1H-pyrazol-5-yl)-4-(3-fluoro-4-(1-methyl-2-(trifluoromethyl)-1H-imidazol-4-yl)benzyl)-6,7-dihydropyrazolo[1,5-α]pyrimidin-5(4H)-one;
(R)-2-(4-chloro-1-(1-methoxypropan-2-yl)-1H-pyrazol-5-yl)-4-(4-(1-ethyl-4-(trifluoromethyl)-1H-imidazol-2-yl)-3-fluorobenzyl)-6,7-dihydropyrazolo[1,5-α]pyrimidin-5(4H)-one;
(S)-2-(4-chloro-1-(1-methoxypropan-2-yl)-1H-pyrazol-5-yl)-4-(4-(1-ethyl-4-(trifluoromethyl)-1H-imidazol-2-yl)-3-fluorobenzyl)-6,7-dihydropyrazolo[1,5-α]pyrimidin-5(4H)-one;
3-(4-((2-(4-chloro-1-isopropyl-1H-pyrazol-5-yl)-5-oxo-6,7-dihydropyrazolo[1,5-α]pyrimidin-4(5H)-yl)methyl)phenyl)-1-methyl-1H-pyrazole-4-carbonitrile;
2-(4-chloro-1-isopropyl-1H-pyrazol-5-yl)-4-(4-(1-ethyl-4-methyl-1H-imidazol-2-yl)benzyl)-6,7-dihydropyrazolo[1,5-α]pyrimidin-5(4H)-one;
2-(4-chloro-1-isopropyl-1H-pyrazol-5-yl)-4-(4-(5-methoxy-3-methyl-1H-pyrazol-1-yl)benzyl)-6,7-dihydropyrazolo[1,5-α]pyrimidin-5(4H)-one;
2-(4-cyclopropyl-6-methoxypyrimidin-5-yl)-4-(4-(1-ethyl-4-(trifluoromethyl)-1H-imidazol-2-yl)-3-methoxybenzyl)-6,7-dihydropyrazolo[1,5-α]pyrimidin-5(4H)-one;
2-(4-cyclopropyl-6-methoxypyrimidin-5-yl)-4-((5-(1-ethyl-4-(trifluoromethyl)-1H-imidazol-2-yl)pyridin-2-yl)methyl)-6,7-dihydropyrazolo[1,5-α]pyrimidin-5(4H)-one;
(S)-2-(4-chloro-1-isopropyl-1H-pyrazol-5-yl)-4-(1-(6-(1-ethyl-4-(trifluoromethyl)-1H-imidazol-2-yl)pyridin-3-yl)ethyl)-6,7-dihydropyrazolo[1,5-α]pyrimidin-5(4H)-one;
(R)-2-(4-chloro-1-isopropyl-1H-pyrazol-5-yl)-4-(1-(6-(1-ethyl-4-(trifluoromethyl)-1H-imidazol-2-yl)pyridin-3-yl)ethyl)-6,7-dihydropyrazolo[1,5-α]pyrimidin-5(4H)-one;
2-(4-cyclopropyl-6-methoxypyrimidin-5-yl)-4-(4-(1-ethyl-4-(trifluoromethyl)-1H-imidazol-2-yl)-3-fluoro-5-methoxybenzyl)-6,7-dihydropyrazolo[1,5-α]pyrimidin-5(4H)-one;
2-(4-chloro-1-isopropyl-1H-pyrazol-5-yl)-4-(4-(1-ethyl-4-(trifluoromethyl)-1H-imidazol-2-yl)-3-fluoro-5-methoxybenzyl)-6,7-dihydropyrazolo[1,5-α]pyrimidin-5(4H)-one;
(S)-2-(4-cyclopropyl-6-methoxypyrimidin-5-yl)-4-(1-(4-(1-ethyl-4-(trifluoromethyl)-1H-imidazol-2-yl)-3-fluorophenyl)ethyl)-6,7-dihydropyrazolo[1,5-α]pyrimidin-5(4H)-one;
(R)-2-(4-cyclopropyl-6-methoxypyrimidin-5-yl)-4-(1-(4-(1-ethyl-4-(trifluoromethyl)-1H-imidazol-2-yl)-3-fluorophenyl)ethyl)-6,7-dihydropyrazolo[1,5-α]pyrimidin-5(4H)-one;
2-(4-cyclopropyl-6-methoxypyrimidin-5-yl)-4-(3-fluoro-4-(1-isopropyl-4-(trifluoromethyl)-1H-imidazol-2-yl)-5-methoxybenzyl)-6,7-dihydropyrazolo[1,5-α]pyrimidin-5(4H)-one; 4-(3-chloro-4-(1-ethyl-4-(trifluoromethyl)-1H-imidazol-2-yl)-5-methoxybenzyl)-2-(4-cyclopropyl-6-methoxypyrimidin-5-yl)-6,7-dihydropyrazolo[1,5-α]pyrimidin-5(4H)-one;
2-(4-chloro-1-isopropyl-1H-pyrazol-5-yl)-4-(4-(3-methoxy-5-methyl-1H-pyrazol-1-yl)benzyl)-6,7-dihydropyrazolo[1,5-α]pyrimidin-5(4H)-one;
3-chloro-2-(1-isopropyl-4-methyl-1H-pyrazol-5-yl)-4-(4-(1-methyl-4-(trifluoromethyl)-1H-imidazol-2-yl)benzyl)-4,5,6,7-tetrahydropyrazolo[1,5-α]pyrimidine;
3-chloro-4-(3-fluoro-4-(1-isopropyl-4-(trifluoromethyl)-1H-imidazol-2-yl)benzyl)-2-(1-isopropyl-4-methyl-1H-pyrazol-5-yl)-4,5,6,7-tetrahydropyrazolo[1,5-α]pyrimidine;
4-(3-fluoro-4-(1-methyl-4-(trifluoromethyl)-1H-imidazol-2-yl)benzyl)-2-(4-methoxy-6-(1-methylcyclopropyl)pyrimidin-5-yl)-6,7-dihydropyrazolo[1,5-α]pyrimidin-5(4H)-one;
2-(4-chloro-1-(tetrahydrofuran-3-yl)-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one;
2-(4-chloro-1-(1-methoxypropan-2-yl)-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one;
4-(4-(1-isopropyl-4-(trifluoromethyl)-1H-imidazol-2-yl)benzyl)-2-(1-isopropyl-4-methoxy-1H-pyrazol-5-yl)-6,7-dihydropyrazolo[1,5-α]pyrimidin-5(4H)-one;
2-(4-chloro-1-(1-(methoxymethyl)cyclopropyl)-1H-pyrazol-5-yl)-4-(4-(1-ethyl-4-(trifluoromethyl)-1H-imidazol-2-yl)-3-fluorobenzyl)-6,7-dihydropyrazolo[1,5-α]pyrimidin-5(4H)-one;
2-(4-chloro-1-(oxetan-3-yl)-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one;
4-(4-(1-ethyl-4-(trifluoromethyl)-1H-imidazol-2-yl)benzyl)-2-(1-isopropyl-4-methoxy-1H-pyrazol-5-yl)-6,7-dihydropyrazolo[1,5-α]pyrimidin-5(4H)-one;
5-(4-(4-(1-ethyl-4-(trifluoromethyl)-1H-imidazol-2-yl)benzyl)-5-oxo-4,5,6,7-tetrahydropyrazolo[1,5-α]pyrimidin-2-yl)-1-isopropyl-1H-pyrazole-4-carbonitrile;
2-(4-(tert-butyl)-1-methyl-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one;
4-(4-(1-ethyl-4-(trifluoromethyl)-1H-imidazol-2-yl)benzyl)-2-(4-isopropyl-1-methyl-1H-pyrazol-5-yl)-6,7-dihydropyrazolo[1,5-α]pyrimidin-5(4H)-one;
2-(4-chloro-1-(1-(methoxymethyl)cyclopropyl)-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one;
2-(4-chloro-1-(1-methoxypropan-2-yl)-1H-pyrazol-5-yl)-4-(4-(1-ethyl-4-(trifluoromethyl)-1H-imidazol-2-yl)-3-fluorobenzyl)-6,7-dihydropyrazolo[1,5-α]pyrimidin-5(4H)-one;
2-(4-cyclopropyl-1-isopropyl-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one;
2-(4-chloro-1-(1-methoxy-2-methylpropan-2-yl)-1H-pyrazol-5-yl)-4-(4-(1-ethyl-4-(trifluoromethyl)-1H-imidazol-2-yl)-3-fluorobenzyl)-6,7-dihydropyrazolo[1,5-α]pyrimidin-5(4H)-one;
2-(4-chloro-1-(2-hydroxyethyl)-1H-pyrazol-5-yl)-4-(4-(1-ethyl-4-(trifluoromethyl)-1H-imidazol-2-yl)-3-fluorobenzyl)-6,7-dihydropyrazolo[1,5-α]pyrimidin-5(4H)-one;
2-(4-chloro-1-isopropyl-1H-pyrazol-5-yl)-4-(4-(1-ethyl-4-(trifluoromethyl)-1H-imidazol-2-yl)phenoxy)-4,5,6,7-tetrahydropyrazolo[1,5-α]pyridine;
2-(4-chloro-1-isopropyl-1H-pyrazol-5-yl)-4-(4-(1-isopropyl-4-(trifluoromethyl)-1H-imidazol-2-yl)-3-methoxybenzyl)-6,7-dihydro-[1,2,4]triazolo[1,5-α]pyrimidin-5(4H)-one;
2-(4-cyclopropyl-6-methoxypyrimidin-5-yl)-4-(4-(1-isopropyl-4-(trifluoromethyl)-1H-imidazol-2-yl)-3-methoxybenzyl)-6,7-dihydro-[1,2,4]triazolo[1,5-α]pyrimidin-5(4H)-one;
2-(4-chloro-1-isopropyl-1H-pyrazol-5-yl)-4-(4-(1-isopropyl-4-(trifluoromethyl)-1H-imidazol-2-yl)-3-(2-methoxyethoxy)benzyl)-6,7-dihydro-[1,2,4]triazolo[1,5-α]pyrimidin-5(4H)-one;
2-(4-cyclopropyl-6-methoxypyrimidin-5-yl)-4-(4-(1-isopropyl-4-(trifluoromethyl)-1H-imidazol-2-yl)benzyl)-6,7-dihydro-[1,2,4]triazolo[1,5-α]pyrimidin-5(4H)-one;
2-(4-cyclopropyl-6-methoxypyrimidin-5-yl)-4-(1-(4-(1-isopropyl-4-(trifluoromethyl)-1H-imidazol-2-yl)phenyl)ethyl)-6,7-dihydro-[1,2,4]triazolo[1,5-α]pyrimidin-5(4H)-one;
2-(4-cyclopropyl-6-methoxypyrimidin-5-yl)-4-(3-fluoro-4-(1-methyl-4-(trifluoromethyl)-1H-imidazol-2-yl)benzyl)-6,7-dihydro-[1,2,4]triazolo[1,5-α]pyrimidin-5(4H)-one;
2-(4-cyclopropyl-6-methoxypyrimidin-5-yl)-4-(4-(1-ethyl-4-(trifluoromethyl)-1H-imidazol-2-yl)benzyl)-6,7-dihydro-[1,2,4]triazolo[1,5-α]pyrimidin-5(4H)-one;
4-(3-chloro-4-(1-isopropyl-4-(trifluoromethyl)-1H-imidazol-2-yl)benzyl)-2-(4-cyclopropyl-6-methoxypyrimidin-5-yl)-6,7-dihydro-[1,2,4]triazolo[1,5-α]pyrimidin-5(4H)-one;
(S)-2-(4-cyclopropyl-6-methoxypyrimidin-5-yl)-4-(1-(3-fluoro-4-(1-isopropyl-4-(trifluoromethyl)-1H-imidazol-2-yl)phenyl)ethyl)-6,7-dihydro-[1,2,4]triazolo[1,5-α]pyrimidin-5(4H)-one;
(R)-2-(4-cyclopropyl-6-methoxypyrimidin-5-yl)-4-(1-(3-fluoro-4-(1-isopropyl-4-(trifluoromethyl)-1H-imidazol-2-yl)phenyl)ethyl)-6,7-dihydro-[1,2,4]triazolo[1,5-α]pyrimidin-5(4H)-one;
4-(3-chloro-4-(1-ethyl-4-(trifluoromethyl)-1H-imidazol-2-yl)benzyl)-2-(4-cyclopropyl-6-methoxypyrimidin-5-yl)-6,7-dihydro-[1,2,4]triazolo[1,5-α]pyrimidin-5(4H)-one;
2-(4-cyclopropyl-6-methoxypyrimidin-5-yl)-4-(4-(1-ethyl-4-(trifluoromethyl)-1H-imidazol-2-yl)-3-fluoro-5-methoxybenzyl)-6,7-dihydro-[1,2,4]triazolo[1,5-α]pyrimidin-5(4H)-one;
(S)-2-(4-chloro-1-isopropyl-1H-pyrazol-5-yl)-4-(1-(4-(1-isopropyl-4-(trifluoromethyl)-1H-imidazol-2-yl)phenyl)ethyl)-6,7-dihydro-[1,2,4]triazolo[1,5-α]pyrimidin-5(4H)-one;
(R)-2-(4-chloro-1-isopropyl-1H-pyrazol-5-yl)-4-(1-(4-(1-isopropyl-4-(trifluoromethyl)-1H-imidazol-2-yl)phenyl)ethyl)-6,7-dihydro-[1,2,4]triazolo[1,5-α]pyrimidin-5(4H)-one;
(S)-2-(4-chloro-1-isopropyl-1H-pyrazol-5-yl)-4-(1-(4-(1-ethyl-4-(trifluoromethyl)-1H-imidazol-2-yl)-3-methoxyphenyl)ethyl)-6,7-dihydro-[1,2,4]triazolo[1,5-α]pyrimidin-5(4H)-one;
(R)-2-(4-chloro-1-isopropyl-1H-pyrazol-5-yl)-4-(1-(4-(1-ethyl-4-(trifluoromethyl)-1H-imidazol-2-yl)-3-methoxyphenyl)ethyl)-6,7-dihydro-[1,2,4]triazolo[1,5-α]pyrimidin-5(4H)-one;
(S)-2-(4-chloro-1-isopropyl-1H-pyrazol-5-yl)-4-(1-(4-(1-ethyl-4-(trifluoromethyl)-1H-imidazol-2-yl)-3-fluorophenyl)ethyl)-6,7-dihydro-[1,2,4]triazolo[1,5-α]pyrimidin-5(4H)-one;
(R)-2-(4-chloro-1-isopropyl-1H-pyrazol-5-yl)-4-(1-(4-(1-ethyl-4-(trifluoromethyl)-1H-imidazol-2-yl)-3-fluorophenyl)ethyl)-6,7-dihydro-[1,2,4]triazolo[1,5-α]pyrimidin-5(4H)-one;
(S)-2-(4-cyclopropyl-6-methoxypyrimidin-5-yl)-4-(1-(4-(1-ethyl-4-(trifluoromethyl)-1H-imidazol-2-yl)phenyl)ethyl)-6,7-dihydro-[1,2,4]triazolo[1,5-α]pyrimidin-5(4H)-one;
(R)-2-(4-cyclopropyl-6-methoxypyrimidin-5-yl)-4-(1-(4-(1-ethyl-4-(trifluoromethyl)-1H-imidazol-2-yl)phenyl)ethyl)-6,7-dihydro-[1,2,4]triazolo[1,5-α]pyrimidin-5(4H)-one;
2-(4-cyclopropyl-6-methoxypyrimidin-5-yl)-4-(4-(1-ethyl-4-(trifluoromethyl)-1H-imidazol-2-yl)-3-methoxybenzyl)-6,7-dihydro-[1,2,4]triazolo[1,5-α]pyrimidin-5(4H)-one;
2-(4-chloro-1-isopropyl-1H-pyrazol-5-yl)-4-((6-(1-ethyl-4-(trifluoromethyl)-1H-imidazol-2-yl)pyridin-3-yl)methyl)-6,7-dihydro-[1,2,4]triazolo[1,5-α]pyrimidin-5(4H)-one;
2-(4-chloro-1-isopropyl-1H-pyrazol-5-yl)-4-(4-(1-ethyl-4-(trifluoromethyl)-1H-imidazol-2-yl)-3-fluoro-5-methoxybenzyl)-6,7-dihydro-[1,2,4]triazolo[1,5-α]pyrimidin-5(4H)-one;
(S)-2-(4-cyclopropyl-6-methoxypyrimidin-5-yl)-4-(1-(4-(1-isopropyl-4-(trifluoromethyl)-1H-imidazol-2-yl)phenyl)ethyl)-6,7-dihydro-[1,2,4]triazolo[1,5-α]pyrimidin-5(4H)-one;
(R)-2-(4-cyclopropyl-6-methoxypyrimidin-5-yl)-4-(1-(4-(1-isopropyl-4-(trifluoromethyl)-1H-imidazol-2-yl)phenyl)ethyl)-6,7-dihydro-[1,2,4]triazolo[1,5-α]pyrimidin-5(4H)-one;
(S)-2-(4-cyclopropyl-6-methoxypyrimidin-5-yl)-4-(1-(4-(1-ethyl-4-(trifluoromethyl)-1H-imidazol-2-yl)-3-fluorophenyl)ethyl)-6,7-dihydro-[1,2,4]triazolo[1,5-α]pyrimidin-5(4H)-one;
(R)-2-(4-cyclopropyl-6-methoxypyrimidin-5-yl)-4-(1-(4-(1-ethyl-4-(trifluoromethyl)-1H-imidazol-2-yl)-3-fluorophenyl)ethyl)-6,7-dihydro-[1,2,4]triazolo[1,5-α]pyrimidin-5(4H)-one;
2-(4-cyclopropyl-6-methoxypyrimidin-5-yl)-4-(3-(difluoromethoxy)-4-(1-ethyl-4-(trifluoromethyl)-1H-imidazol-2-yl)benzyl)-6,7-dihydro-[1,2,4]triazolo[1,5-α]pyrimidin-5(4H)-one;
(S)-2-(4-chloro-1-isopropyl-1H-pyrazol-5-yl)-4-(1-(4-(1-ethyl-4-(trifluoromethyl)-1H-imidazol-2-yl)phenyl)ethyl)-6,7-dihydro-[1,2,4]triazolo[1,5-α]pyrimidin-5(4H)-one;
(R)-2-(4-chloro-1-isopropyl-1H-pyrazol-5-yl)-4-(1-(4-(1-ethyl-4-(trifluoromethyl)-1H-imidazol-2-yl)phenyl)ethyl)-6,7-dihydro-[1,2,4]triazolo[1,5-α]pyrimidin-5(4H)-one;
2-(4-cyclopropyl-6-methoxypyrimidin-5-yl)-4-(4-(1-(1-fluoropropan-2-yl)-4-(trifluoromethyl)-1H-imidazol-2-yl)benzyl)-6,7-dihydro-[1,2,4]triazolo[1,5-α]pyrimidin-5(4H)-one;
(S)-2-(4-cyclopropyl-6-methoxypyrimidin-5-yl)-4-(1-(3-fluoro-4-(1-methyl-4-(trifluoromethyl)-1H-imidazol-2-yl)phenyl)ethyl)-6,7-dihydro-[1,2,4]triazolo[1,5-α]pyrimidin-5(4H)-one;
(R)-2-(4-cyclopropyl-6-methoxypyrimidin-5-yl)-4-(1-(3-fluoro-4-(1-methyl-4-(trifluoromethyl)-1H-imidazol-2-yl)phenyl)ethyl)-6,7-dihydro-[1,2,4]triazolo[1,5-α]pyrimidin-5(4H)-one;
(S)-2-(4-(1-(2-(4-cyclopropyl-6-methoxypyrimidin-5-yl)-5-oxo-6,7-dihydro-[1,2,4]triazolo[1,5-α]pyrimidin-4(5H)-yl)ethyl)phenyl)-1-ethyl-1H-imidazole-4-carbonitrile;
(R)-2-(4-(1-(2-(4-cyclopropyl-6-methoxypyrimidin-5-yl)-5-oxo-6,7-dihydro-[1,2,4]triazolo[1,5-α]pyrimidin-4(5H)-yl)ethyl)phenyl)-1-ethyl-1H-imidazole-4-carbonitrile;
(S)-4-(1-(4-(1-cyclopropyl-4-(trifluoromethyl)-1H-imidazol-2-yl)phenyl)ethyl)-2-(4-cyclopropyl-6-methoxypyrimidin-5-yl)-6,7-dihydro-[1,2,4]triazolo[1,5-α]pyrimidin-5(4H)-one;
(R)-4-(1-(4-(1-cyclopropyl-4-(trifluoromethyl)-1H-imidazol-2-yl)phenyl)ethyl)-2-(4-cyclopropyl-6-methoxypyrimidin-5-yl)-6,7-dihydro-[1,2,4]triazolo[1,5-α]pyrimidin-5(4H)-one;
2-(4-cyclopropyl-6-methoxypyrimidin-5-yl)-4-(5-fluoro-4-(1-isopropyl-4-(trifluoromethyl)-1H-imidazol-2-yl)-2-methoxybenzyl)-6,7-dihydro-[1,2,4]triazolo[1,5-α]pyrimidin-5(4H)-one;
(S)-2-(4-cyclopropyl-6-methoxypyrimidin-5-yl)-4-(1-(3-fluoro-4-(5-methyl-3-(trifluoromethyl)-1H-pyrazol-1-yl)phenyl)ethyl)-6,7-dihydro-[1,2,4]triazolo[1,5-α]pyrimidin-5(4H)-one;
(R)-2-(4-cyclopropyl-6-methoxypyrimidin-5-yl)-4-(1-(3-fluoro-4-(5-methyl-3-(trifluoromethyl)-1H-pyrazol-1-yl)phenyl)ethyl)-6,7-dihydro-[1,2,4]triazolo[1,5-α]pyrimidin-5(4H)-one;
(S)-4-(1-(4-(5-bromo-1-methyl-4-(trifluoromethyl)-1H-imidazol-2-yl)phenyl)ethyl)-2-(4-cyclopropyl-6-methoxypyrimidin-5-yl)-6,7-dihydro-[1,2,4]triazolo[1,5-α]pyrimidin-5(4H)-one;
(R)-4-(1-(4-(5-bromo-1-methyl-4-(trifluoromethyl)-1H-imidazol-2-yl)phenyl)ethyl)-2-(4-cyclopropyl-6-methoxypyrimidin-5-yl)-6,7-dihydro-[1,2,4]triazolo[1,5-α]pyrimidin-5(4H)-one;
2-(2-cyclopropyl-4-methoxypyridin-3-yl)-4-(3-fluoro-4-(1-isopropyl-4-(trifluoromethyl)-1H-imidazol-2-yl)benzyl)-6,7-dihydro-[1,2,4]triazolo[1,5-α]pyrimidin-5(4H)-one;
(R)-2-(4-cyclopropyl-6-(difluoromethoxy)pyrimidin-5-yl)-4-(1-(4-(1-ethyl-4-(trifluoromethyl)-1H-imidazol-2-yl)phenyl)ethyl)-6,7-dihydro-[1,2,4]triazolo[1,5-α]pyrimidin-5(4H)-one;
(S)-2-(4-cyclopropyl-6-(difluoromethoxy)pyrimidin-5-yl)-4-(1-(4-(1-ethyl-4-(trifluoromethyl)-1H-imidazol-2-yl)phenyl)ethyl)-6,7-dihydro-[1,2,4]triazolo[1,5-α]pyrimidin-5(4H)-one;
2-(4-cyclopropyl-6-methoxypyrimidin-5-yl)-4-(3-fluoro-4-(1-isopropyl-4-(trifluoromethyl)-1H-imidazol-2-yl)-2-methoxybenzyl)-6,7-dihydro-[1,2,4]triazolo[1,5-α]pyrimidin-5(4H)-one;
(R)-4-(1-(3-chloro-4-(1-ethyl-4-(trifluoromethyl)-1H-imidazol-2-yl)phenyl)ethyl)-2-(4-cyclopropyl-6-methoxypyrimidin-5-yl)-6,7-dihydro-[1,2,4]triazolo[1,5-α]pyrimidin-5(4H)-one;
(S)-4-(1-(3-chloro-4-(1-ethyl-4-(trifluoromethyl)-1H-imidazol-2-yl)phenyl)ethyl)-2-(4-cyclopropyl-6-methoxypyrimidin-5-yl)-6,7-dihydro-[1,2,4]triazolo[1,5-α]pyrimidin-5(4H)-one;
2-(4-cyclopropyl-2-methoxypyridin-3-yl)-4-(3-fluoro-4-(1-isopropyl-4-(trifluoromethyl)-1H-imidazol-2-yl)benzyl)-6,7-dihydro-[1,2,4]triazolo[1,5-α]pyrimidin-5(4H)-one;
2-(4-cyclopropyl-6-methoxypyrimidin-5-yl)-4-(1-(4-(5-(2,2-difluoroethoxy)-3-(trifluoromethyl)-1H-pyrazol-1-yl)phenyl)ethyl)-6,7-dihydro-[1,2,4]triazolo[1,5-α]pyrimidin-5(4H)-one;
2-(4-chloro-1-isopropyl-1H-pyrazol-5-yl)-4-(1-(4-(5-(2,2-difluoroethoxy)-3-(trifluoromethyl)-1H-pyrazol-1-yl)phenyl)ethyl)-6,7-dihydro-[1,2,4]triazolo[1,5-α]pyrimidin-5(4H)-one;
2-(4-chloro-1-isopropyl-1H-pyrazol-5-yl)-4-(3-fluoro-4-(1-isopropyl-4-(trifluoromethyl)-1H-imidazol-2-yl)-2-methoxybenzyl)-6,7-dihydro-[1,2,4]triazolo[1,5-α]pyrimidin-5(4H)-one;
2-(4-cyclopropyl-6-methoxypyrimidin-5-yl)-4-(4-(1-ethyl-4-(trifluoromethyl)-1H-imidazol-2-yl)-3-fluoro-2-methoxybenzyl)-6,7-dihydro-[1,2,4]triazolo[1,5-α]pyrimidin-5(4H)-one;
2-(4-cyclopropyl-6-methoxypyrimidin-5-yl)-4-(1-(3-fluoro-4-(1-isopropyl-4-(trifluoromethyl)-1H-imidazol-2-yl)-2-methoxyphenyl)ethyl)-6,7-dihydro-[1,2,4]triazolo[1,5-α]pyrimidin-5(4H)-one;
2-(4-cyclopropyl-6-methoxypyrimidin-5-yl)-4-(1-(4-(3-(dimethylamino)pyridin-2-yl)phenyl)ethyl)-6,7-dihydro-[1,2,4]triazolo[1,5-α]pyrimidin-5(4H)-one;
2-(4-cyclopropyl-6-methoxypyrimidin-5-yl)-4-(1-(4-(3-isopropoxypyridin-2-yl)phenyl)ethyl)-6,7-dihydro-[1,2,4]triazolo[1,5-α]pyrimidin-5(4H)-one;
2-(1-isopropyl-4-methyl-1H-pyrazol-5-yl)-4-(4-(1-methyl-4-(trifluoromethyl)-1H-imidazol-2-yl)benzyl)-4,5,6,7-tetrahydro-[1,2,4]triazolo[1,5-α]pyrimidine;
4-(4-(1-isopropyl-4-(trifluoromethyl)-1H-imidazol-2-yl)benzyl)-2-(1-isopropyl-4-methyl-1H-pyrazol-5-yl)-4,5,6,7-tetrahydro-[1,2,4]triazolo[1,5-α]pyrimidine;
2-(4-chloro-1-isopropyl-1H-pyrazol-5-yl)-4-(4-(1-ethyl-4-(trifluoromethyl)-1H-imidazol-2-yl)benzyl)-5-oxo-4,5,6,7-tetrahydropyrazolo[1,5-α]pyrimidine-3-carbonitrile;
2-(4-chloro-1-isopropyl-1H-pyrazol-5-yl)-4-(4-(5-((methylamino)methyl)-3-(trifluoromethyl)-1H-pyrazol-1-yl)benzyl)-6,7-dihydropyrazolo[1,5-α]pyrimidin-5(4H)-one;
2-(4-cyclopropyl-6-methoxypyrimidin-5-yl)-4-(4-(5-((methylamino)methyl)-3-(trifluoromethyl)-1H-pyrazol-1-yl)benzyl)-6,7-dihydropyrazolo[1,5-α]pyrimidin-5(4H)-one;
2-(4-chloro-1-isopropyl-1H-pyrazol-5-yl)-4-(4-(pyridin-2-ylmethoxy)benzyl)-6,7-dihydropyrazolo[1,5-α]pyrimidin-5(4H)-one;
2-(4-chloro-1-(1-hydroxypropan-2-yl)-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one;
2-(4-chloro-1-isopropyl-1H-pyrazol-5-yl)-4-(3-(2-hydroxyethoxy)-4-(1-isopropyl-4-(trifluoromethyl)-1H-imidazol-2-yl)benzyl)-6,7-dihydro-[1,2,4]triazolo[1,5-α]pyrimidin-5(4H)-one;
3-amino-2-(4-chloro-1-isopropyl-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one;
2-(4-chloro-1-isopropyl-1H-pyrazol-5-yl)-4-(4-(3-morpholino-1H-pyrazol-1-yl)benzyl)-6,7-dihydropyrazolo[1,5-α]pyrimidin-5(4H)-one;
2-(4-chloro-1-isopropyl-1H-pyrazol-5-yl)-6-hydroxy-4-(4-(1-methyl-4-(trifluoromethyl)-1H-imidazol-2-yl)benzyl)-6,7-dihydropyrazolo[1,5-α]pyrimidin-5(4H)-one;
2-(4-chloro-1-isopropyl-1H-pyrazol-5-yl)-6-hydroxy-6-methyl-4-(4-(1-methyl-4-(trifluoromethyl)-1H-imidazol-2-yl)benzyl)-6,7-dihydropyrazolo[1,5-α]pyrimidin-5(4H)-one;
6-amino-2-(4-chloro-1-isopropyl-1H-pyrazol-5-yl)-4-(4-(1-ethyl-4-(trifluoromethyl)-1H-imidazol-2-yl)benzyl)-6,7-dihydropyrazolo[1,5-α]pyrimidin-5(4H)-one;
N-(2-(4-chloro-1-isopropyl-1H-pyrazol-5-yl)-4-(4-(1-ethyl-4-(trifluoromethyl)-1H-imidazol-2-yl)benzyl)-5-oxo-4,5,6,7-tetrahydropyrazolo[1,5-α]pyrimidin-6-yl)acetamide;
2-(4-chloro-1-isopropyl-1H-pyrazol-5-yl)-6-(dimethylamino)-4-(4-(1-ethyl-4-(trifluoromethyl)-1H-imidazol-2-yl)benzyl)-6,7-dihydropyrazolo[1,5-α]pyrimidin-5(4H)-one;
2-(4-chloro-1-isopropyl-1H-pyrazol-5-yl)-4-(4-(1-ethyl-4-(trifluoromethyl)-1H-imidazol-2-yl)benzyl)-6-(methylamino)-6,7-dihydropyrazolo[1,5-α]pyrimidin-5(4H)-one;
2-(4-chloro-1-isopropyl-1H-pyrazol-5-yl)-N-(4-(1-ethyl-4-(trifluoromethyl)-1H-imidazol-2-yl)phenyl)-4,5,6,7-tetrahydropyrazolo[1,5-α]pyridin-4-amine;
(R)-2-(4-chloro--isopropyl-1H-pyrazol-5-yl)-N-(4-(1-ethyl-4-(trifluoromethyl)-1H-imidazol-2-yl)phenyl)-4,5,6,7-tetrahydropyrazolo[1,5-α]pyridin-4-amine;
(S)-2-(4-chloro-1-isopropyl-1H-pyrazol-5-yl)-N-(4-(1-ethyl-4-(trifluoromethyl)-1H-imidazol-2-yl)phenyl)-4,5,6,7-tetrahydropyrazolo[1,5-α]pyridin-4-amine;
2-(4-cyclopropyl-6-methoxypyrimidin-5-yl)-N-(4-(1-ethyl-4-(trifluoromethyl)-1H-imidazol-2-yl)phenyl)-4,5,6,7-tetrahydropyrazolo[1,5-α]pyridin-4-amine;
2-(4-chloro-1-isopropyl-1H-pyrazol-5-yl)-N-(4-(1-ethyl-4-(trifluoromethyl)-1H-imidazol-2-yl)-3-fluorophenyl)-4,5,6,7-tetrahydropyrazolo[1,5-α]pyridin-4-amine;
(S)-2-(4-cyclopropyl-6-methoxypyrimidin-5-yl)-N-(4-(1-ethyl-4-(trifluoromethyl)-1H-imidazol-2-yl)phenyl)-4,5,6,7-tetrahydropyrazolo[1,5-α]pyridin-4-amine;
(R)-2-(4-cyclopropyl-6-methoxypyrimidin-5-yl)-N-(4-(1-ethyl-4-(trifluoromethyl)-1H-imidazol-2-yl)phenyl)-4,5,6,7-tetrahydropyrazolo[1,5-α]pyridin-4-amine;
2-(4-chloro-1-isopropyl-1H-pyrazol-5-yl)-N-(4-(5-methyl-3-(trifluoromethyl)-1H-pyrazol-1-yl)phenyl)-4,5,6,7-tetrahydropyrazolo[1,5-α]pyridin-4-amine;
2-(4-chloro-1-isopropyl-1H-pyrazol-5-yl)-N-(6-(1-ethyl-4-(trifluoromethyl)-1H-imidazol-2-yl)pyridin-3-yl)-4,5,6,7-tetrahydropyrazolo[1,5-α]pyridin-4-amine;
2-(4-chloro-1-isopropyl-1H-pyrazol-5-yl)-N-(4-(1-ethyl-4-(trifluoromethyl)-1H-imidazol-2-yl)-3-methoxyphenyl)-4, 5, 6,7-tetrahydropyrazolo[1,5-α]pyridin-4-amine;
2-(4-((2-(4-chloro-1-isopropyl-1H-pyrazol-5-yl)-4,5,6,7-tetrahydropyrazolo[1,5-α]pyridin-4-yl)amino)phenyl)-1-ethyl-1H-imidazole-4-carbonitrile;
2-(4-cyclopropyl-6-methoxypyrimidin-5-yl)-N-(4-(1-(1-fluoropropan-2-yl)-4-(trifluoromethyl)-1H-imidazol-2-yl)phenyl)-4,5,6,7-tetrahydropyrazolo[1,5-α]pyridin-4-amine;
2-(4-cyclopropyl-6-(difluoromethoxy)pyrimidin-5-yl)-N-(4-(1-(1-fluoropropan-2-yl)-4-(trifluoromethyl)-1H-imidazol-2-yl)phenyl)-4,5,6,7-tetrahydropyrazolo[1,5-α]pyridin-4-amine;
2-(4-cyclopropyl-6-(difluoromethoxy)pyrimidin-5-yl)-N-(4-(1-ethyl-4-(trifluoromethyl)-1H-imidazol-2-yl)phenyl)-4,5,6,7-tetrahydropyrazolo[1,5-α]pyridin-4-amine;
(R)-2-(4-cyclopropyl-6-methoxypyrimidin-5-yl)-N-(3-fluoro-4-(3-methoxypyridin-2-yl)phenyl)-4,5,6,7-tetrahydropyrazolo[1,5-α]pyridin-4-amine;
(S)-2-(4-cyclopropyl-6-methoxypyrimidin-5-yl)-N-(3-fluoro-4-(3-methoxypyridin-2-yl)phenyl)-4,5,6,7-tetrahydropyrazolo[1,5-α]pyridin-4-amine;
(R)-N-(3-chloro-4-(3-methoxypyridin-2-yl)phenyl)-2-(4-cyclopropyl-6-methoxypyrimidin-5-yl)-4,5,6,7-tetrahydropyrazolo[1,5-α]pyridin-4-amine;
(S)-N-(3-chloro-4-(3-methoxypyridin-2-yl)phenyl)-2-(4-cyclopropyl-6-methoxypyrimidin-5-yl)-4,5,6,7-tetrahydropyrazolo[1,5-α]pyridin-4-amine;
5-((2-(4-chloro-1-isopropyl-1H-pyrazol-5-yl)-4,5,6,7-tetrahydropyrazolo[1,5-α]pyridin-4-yl)amino)-2-(1-ethyl-4-(trifluoromethyl)-1H-imidazol-2-yl)benzonitrile;
2-(4-chloro-1-isopropyl-1H-pyrazol-5-yl)-N-(4-(1-ethyl-4-(trifluoromethyl)-1H-imidazol-2-yl)phenyl)-5,6,7,8-tetrahydro-[1,2,4]triazolo[1,5-α]pyridin-8-amine;
2-(4-chloro-1-isopropyl-1H-pyrazol-5-yl)-N-(4-(1-ethyl-4-(trifluoromethyl)-1H-imidazol-2-yl)-3-fluorophenyl)-5,6,7,8-tetrahydro-[1,2,4]triazolo[1,5-α]pyridin-8-amine;
2-(4-chloro-1-isopropyl-1H-pyrazol-5-yl)-N-(4-(1-ethyl-4-(trifluoromethyl)-1H-imidazol-2-yl)phenyl)-N-methyl-4,5,6,7-tetrahydropyrazolo[1,5-α]pyridin-4-amine;
2-(4-chloro-1-isopropyl-1H-pyrazol-5-yl)-N-(4-(1-ethyl-4-(trifluoromethyl)-1H-imidazol-2-yl)-3-fluorophenyl)-N-methyl-5,6,7,8-tetrahydro-[1,2,4]triazolo[1,5-α]pyridin-8-amine;
2-(4-chloro-1-isopropyl-1H-pyrazol-5-yl)-N-(4-(1-ethyl-4-(trifluoromethyl)-1H-imidazol-2-yl)phenyl)-N-methyl-5,6,7,8-tetrahydro-[1,2,4]triazolo[1,5-α]pyridin-8-amine;
2-(4-chloro-1-isopropyl-1H-pyrazol-5-yl)-4-(4-(1-ethyl-4-(trifluoromethyl)-1H-imidazol-2-yl)phenoxy)-4,5,6,7-tetrahydropyrazolo[1,5-α]pyridine;
2-(4-chloro-1-isopropyl-1H-pyrazol-5-yl)-4-(4-(1-ethyl-4-(trifluoromethyl)-1H-imidazol-2-yl)-3-fluorophenoxy)-4,5,6,7-tetrahydropyrazolo[1,5-α]pyridine;
(S)-2-(4-cyclopropyl-6-methoxypyrimidin-5-yl)-4-(4-(1-ethyl-4-(trifluoromethyl)-1H-imidazol-2-yl)-3-fluorophenoxy)-4,5,6,7-tetrahydropyrazolo[1,5-α]pyridine;
(R)-2-(4-cyclopropyl-6-methoxypyrimidin-5-yl)-4-(4-(1-ethyl-4-(trifluoromethyl)-1H-imidazol-2-yl)-3-fluorophenoxy)-4,5,6,7-tetrahydropyrazolo[1,5-α]pyridine;
2-(4-chloro-1-isopropyl-1H-pyrazol-5-yl)-4-(4-(1-ethyl-4-(trifluoromethyl)-1H-imidazol-2-yl)benzyl)-6,7-dihydro-4H-pyrazolo[5, 1-c][1,4] oxazine;
5-(4-((4-(1-ethyl-4-(trifluoromethyl)-1H-imidazol-2-yl)-3-fluorophenyl)amino)-4,5,6,7-tetrahydropyrazolo[1,5-α]pyridin-2-yl)-1-isopropyl-1H-pyrazole-4-carbonitrile;
2-(4-chloro-1-isopropyl-1H-pyrazol-5-yl)-4-(4-(1-ethyl-4-(trifluoromethyl)-1H-imidazol-2-yl)-3-fluorobenzyl)-4,5,6,7-tetrahydropyrazolo[1,5-α]pyrimidin-6-ol ;
3-chloro-4-(4-(1-ethyl-4-(trifluoromethyl)-1H-imidazol-2-yl)benzyl)-2-(1-isopropyl-1H-pyrazol-5-yl)-6,7-dihydropyrazolo[1,5-α]pyrimidin-5(4H)-one;
3-chloro-N-(4-(1-ethyl-4-(trifluoromethyl)-1H-imidazol-2-yl)-3-fluorophenyl)-2-(1-isopropyl-1H-pyrazol-5-yl)-4,5,6,7-tetrahydropyrazolo[1,5-α]pyridin-4-amine;
2-(4-chloro-1-isopropyl-1H-pyrazol-5-yl)-4-(3-chloro-4-(3-(difluoromethoxy)pyridin-2-yl)benzyl)-6,7-dihydropyrazolo[1,5-α]pyrimidin-5(4H)-one;
2-(4-chloro-1-isopropyl-1H-pyrazol-5-yl)-4-(3-chloro-4-(6-(trifluoromethyl)pyrazin-2-yl)benzyl)-6,7-dihydropyrazolo[1,5-α]pyrimidin-5(4H)-one;
2-(4-chloro-1-isopropyl-1H-pyrazol-5-yl)-4-(3-chloro-4-(4-(trifluoromethyl)pyrimidin-2-yl)benzyl)-6,7-dihydropyrazolo[1,5-α]pyrimidin-5(4H)-one;
2-(4-chloro-1-isopropyl-1H-pyrazol-5-yl)-4-(3-chloro-4-(3-methoxypyrazin-2-yl)benzyl)-6,7-dihydropyrazolo[1,5-α]pyrimidin-5(4H)-one;
2-(4-chloro-1-isopropyl-1H-pyrazol-5-yl)-4-(3-chloro-4-(2-methylimidazo[1,2-α]pyrazin-8-yl)benzyl)-6,7-dihydropyrazolo[1,5-α]pyrimidin-5(4H)-one;
2-(4-chloro-1-isopropyl-1H-pyrazol-5-yl)-4-(3-chloro-4-(pyrrolo[2,1-f][1,2,4]triazin-4-yl)benzyl)-6,7-dihydropyrazolo[1,5-α]pyrimidin-5(4H)-one;
2-(4-chloro-1-isopropyl-1H-pyrazol-5-yl)-4-(3-chloro-4-(1,2-dimethyl-1H-imidazol-4-yl)benzyl)-6,7-dihydropyrazolo[1,5-α]pyrimidin-5(4H)-one;
2-(4-chloro-1-isopropyl-1H-pyrazol-5-yl)-4-(3-chloro-4-(4-methylpyridazin-3-yl)benzyl)-6,7-dihydropyrazolo[1,5-α]pyrimidin-5(4H)-one;
2-(4-chloro-1-isopropyl-1H-pyrazol-5-yl)-4-(3-chloro-4-(3-(trifluoromethoxy)pyridin-2-yl)benzyl)-6,7-dihydropyrazolo[1,5-α]pyrimidin-5(4H)-one;
2-(4-chloro-1-isopropyl-1H-pyrazol-5-yl)-4-(3-chloro-4-(5-methylpyrrolo[2,1-f][1,2,4]triazin-4-yl)benzyl)-6,7-dihydropyrazolo[1,5-α]pyrimidin-5(4H)-one;
2-(4-chloro-1-isopropyl-1H-pyrazol-5-yl)-4-(3-chloro-4-(1,3-dimethyl-1H-pyrazol-5-yl)benzyl)-6,7-dihydropyrazolo[1,5-α]pyrimidin-5(4H)-one;
2-(4-chloro-1-isopropyl-1H-pyrazol-5-yl)-4-(3-chloro-4-(3-fluoro-6-methylpyridin-2-yl)benzyl)-6,7-dihydropyrazolo[1,5-α]pyrimidin-5(4H)-one;
2-(4-chloro-1-isopropyl-1H-pyrazol-5-yl)-4-(3-chloro-4-(3-(trifluoromethyl)pyridin-2-yl)benzyl)-6,7-dihydropyrazolo[1,5-α]pyrimidin-5(4H)-one;
2-(4-chloro-1-isopropyl-1H-pyrazol-5-yl)-4-(3-chloro-4-(1,2-dimethyl-1H-imidazol-5-yl)benzyl)-6,7-dihydropyrazolo[1,5-α]pyrimidin-5(4H)-one;
2-(4-chloro-1-isopropyl-1H-pyrazol-5-yl)-4-(3-chloro-4-(5-methoxypyridin-2-yl)benzyl)-6,7-dihydropyrazolo[1,5-α]pyrimidin-5(4H)-one;
2-(4-chloro-1-isopropyl-1H-pyrazol-5-yl)-4-(3-chloro-4-(4-methoxypyrimidin-2-yl)benzyl)-6,7-dihydropyrazolo[1,5-α]pyrimidin-5(4H)-one;
3-(2-chloro-4-((2-(4-chloro-1-isopropyl-1H-pyrazol-5-yl)-5-oxo-6,7-dihydropyrazolo[1,5-α]pyrimidin-4(5H)-yl)methyl)phenyl)-1-methyl-1H-pyrazole-4-carbonitrile;
2-(4-chloro-1-isopropyl-1H-pyrazol-5-yl)-4-(3-chloro-4-(3-methoxypyridin-2-yl)benzyl)-6,7-dihydropyrazolo[1,5-α]pyrimidin-5(4H)-one;
2-(4-chloro-1-isopropyl-1H-pyrazol-5-yl)-4-(3-chloro-4-(1,3,5-trimethyl-1H-pyrazol-4-yl)benzyl)-6,7-dihydropyrazolo[1,5-α]pyrimidin-5(4H)-one;
2-(4-chloro-1-isopropyl-1H-pyrazol-5-yl)-4-(3-chloro-4-(3-cyclopropoxypyridin-2-yl)benzyl)-6,7-dihydropyrazolo[1,5-α]pyrimidin-5(4H)-one;
2-(4-chloro-1-isopropyl-1H-pyrazol-5-yl)-4-(4-(3-isopropoxypyridin-2-yl)benzyl)-6,7-dihydropyrazolo[1,5-α]pyrimidin-5(4H)-one;
2-(4-chloro-1-isopropyl-1H-pyrazol-5-yl)-4-(3-chloro-4-(4-methoxy-1-methyl-1H-pyrazol-3-yl)benzyl)-6,7-dihydropyrazolo[1,5-α]pyrimidin-5(4H)-one;
2-(4-chloro-1-isopropyl-1H-pyrazol-5-yl)-4-(3-chloro-4-(4-methoxypyridazin-3-yl)benzyl)-6,7-dihydropyrazolo[1,5-α]pyrimidin-5(4H)-one;
2-(4-chloro-1-isopropyl-1H-pyrazol-5-yl)-4-(3-chloro-4-(3-methoxy-6-(trifluoromethyl)pyridin-2-yl)benzyl)-6,7-dihydropyrazolo[1,5-α]pyrimidin-5(4H)-one;
4-(3-chloro-4-(3-isopropoxypyridin-2-yl)benzyl)-2-(4-cyclopropyl-6-methoxypyrimidin-5-yl)-6,7-dihydro-[1,2,4]triazolo[1,5-α]pyrimidin-5(4H)-one;
2-(4-chloro-1-isopropyl-1H-pyrazol-5-yl)-4-(3-chloro-4-(3-morpholinopyridin-2-yl)benzyl)-6,7-dihydropyrazolo[1,5-α]pyrimidin-5(4H)-one;
2-(4-chloro-1-isopropyl-1H-pyrazol-5-yl)-4-(3-chloro-4-(4-fluoro-1-isopropyl-1H-imidazol-2-yl)benzyl)-6,7-dihydropyrazolo[1,5-α]pyrimidin-5(4H)-one;
2-(4-chloro-1-isopropyl-1H-pyrazol-5-yl)-4-(3-chloro-4-(3-methoxy-4-methylpyridin-2-yl)benzyl)-6,7-dihydropyrazolo[1,5-α]pyrimidin-5(4H)-one;
2-(4-chloro-1-isopropyl-1H-pyrazol-5-yl)-4-(3-chloro-4-(4-methyl-3,4-dihydro-2H-pyrido[4,3-b][1,4] oxazin-5-yl)benzyl)-6,7-dihydropyrazolo[1,5-α]pyrimidin-5(4H)-one;
2-(4-chloro-1-isopropyl-1H-pyrazol-5-yl)-4-(3-chloro-4-(3-(methoxymethyl)pyridin-2-yl)benzyl)-6,7-dihydropyrazolo[1,5-α]pyrimidin-5(4H)-one;
2-(4-chloro-1-isopropyl-1H-pyrazol-5-yl)-4-(3-chloro-4-(3-methoxy-5-methylpyridin-2-yl)benzyl)-6,7-dihydropyrazolo[1,5-α]pyrimidin-5(4H)-one;
2-(4-chloro-1-isopropyl-1H-pyrazol-5-yl)-4-(3-chloro-4-(3-isopropoxypyridin-2-yl)benzyl)-6,7-dihydro-[1,2,4]triazolo[1,5-α]pyrimidin-5(4H)-one;
2-(4-chloro-1-isopropyl-1H-pyrazol-5-yl)-4-(3-chloro-4-(1-isopropyl-1H-imidazol-2-yl)benzyl)-6,7-dihydropyrazolo[1,5-α]pyrimidin-5(4H)-one;
2-(4-chloro-1-isopropyl-1H-pyrazol-5-yl)-4-(4-(3-isopropoxypyridin-2-yl)benzyl)-6,7-dihydro-[1,2,4]triazolo[1,5-α]pyrimidin-5(4H)-one;
2-(4-chloro-1-isopropyl-1H-pyrazol-5-yl)-4-(4-(3-methoxypyridin-2-yl)benzyl)-6,7-dihydropyrazolo[1,5-α]pyrimidin-5(4H)-one;
4-(3-chloro-4-(3-methoxypyridin-2-yl)benzyl)-2-(4-cyclopropyl-6-methoxypyrimidin-5-yl)-6,7-dihydro-[1,2,4]triazolo[1,5-α]pyrimidin-5(4H)-one;
2-(4-chloro-1-isopropyl-1H-pyrazol-5-yl)-4-(3-chloro-4-(3-ethoxypyridin-2-yl)benzyl)-6,7-dihydropyrazolo[1,5-α]pyrimidin-5(4H)-one;
2-(4-cyclopropyl-6-methoxypyrimidin-5-yl)-4-(3-fluoro-4-(3-methoxypyridin-2-yl)benzyl)-6,7-dihydro-[1,2,4]triazolo[1,5-α]pyrimidin-5(4H)-one;
2-(4-chloro-1-isopropyl-1H-pyrazol-5-yl)-4-(3-chloro-4-(3-methoxy-6-methylpyridin-2-yl)benzyl)-6,7-dihydropyrazolo[1,5-α]pyrimidin-5(4H)-one;
2-(4-chloro-1-isopropyl-1H-pyrazol-5-yl)-4-(3-chloro-4-(3-isopropoxypyridin-2-yl)benzyl)-6,7-dihydropyrazolo[1,5-α]pyrimidin-5(4H)-one;
2-(4-chloro-1-isopropyl-1H-pyrazol-5-yl)-4-(4-(3-chloropyridin-2-yl)-3-methoxybenzyl)-6,7-dihydropyrazolo[1,5-α]pyrimidin-5(4H)-one;
2-(4-chloro-1-isopropyl-1H-pyrazol-5-yl)-4-(3-chloro-4-(3-(dimethylamino)pyridin-2-yl)benzyl)-6,7-dihydropyrazolo[1,5-α]pyrimidin-5(4H)-one;
4-(3-chloro-4-(3-methoxypyridin-2-yl)benzyl)-2-(4-cyclopropyl-6-methoxypyrimidin-5-yl)-6,7-dihydropyrazolo[1,5-α]pyrimidin-5(4H)-one;
2-(4-chloro-1-isopropyl-1H-pyrazol-5-yl)-4-(3-chloro-4-(6-(ethylamino)pyridazin-3-yl)benzyl)-6,7-dihydropyrazolo[1,5-α]pyrimidin-5(4H)-one;
2-(4-chloro-1-isopropyl-1H-pyrazol-5-yl)-4-(3-chloro-4-(1-isopropyl-1H-imidazol-4-yl)benzyl)-6,7-dihydropyrazolo[1,5-α]pyrimidin-5(4H)-one;
2-(4-chloro-1-isopropyl-1H-pyrazol-5-yl)-4-(3-chloro-4-(3-fluoro-6-(trifluoromethyl)pyridin-2-yl)benzyl)-6,7-dihydropyrazolo[1,5-α]pyrimidin-5(4H)-one;
2-(4-cyclopropyl-6-methoxypyrimidin-5-yl)-4-(3-fluoro-4-(3-methoxypyridin-2-yl)benzyl)-6,7-dihydropyrazolo[1,5-α]pyrimidin-5(4H)-one;
2-(4-chloro-1-isopropyl-1H-pyrazol-5-yl)-4-(3-chloro-4-(5-methoxypyrimidin-4-yl)benzyl)-6,7-dihydropyrazolo[1,5-α]pyrimidin-5(4H)-one;
2-(4-chloro-1-isopropyl-1H-pyrazol-5-yl)-4-(3-chloro-4-(6-methoxypyridazin-3-yl)benzyl)-6,7-dihydropyrazolo[1,5-α]pyrimidin-5(4H)-one;
3-(2-chloro-4-((2-(4-chloro-1-isopropyl-1H-pyrazol-5-yl)-5-oxo-6,7-dihydropyrazolo[1,5-α]pyrimidin-4(5H)-yl)methyl)phenyl)-N,N-dimethyl-1H-pyrazole-1-sulfonamide;
2-(4-chloro-1-isopropyl-1H-pyrazol-5-yl)-4-(4-(3-(dimethylamino)pyridin-2-yl)benzyl)-6,7-dihydropyrazolo[1,5-α]pyrimidin-5(4H)-one;
2-(4-chloro-1-isopropyl-1H-pyrazol-5-yl)-4-(4-(3-(2,2-difluoroethoxy)pyridin-2-yl)benzyl)-6,7-dihydro-[1,2,4]triazolo[1,5-α]pyrimidin-5(4H)-one;
2-(4-chloro-1-((R)-1-fluoropropan-2-yl)-1H-pyrazol-5-yl)-N-(4-(1-ethyl-4-(trifluoromethyl)-1H-imidazol-2-yl)phenyl)-4,5,6,7-tetrahydropyrazolo[1,5-α]pyridin-4-amine;
2-(4-chloro-1-((S)-1-fluoropropan-2-yl)-1H-pyrazol-5-yl)-N-(4-(1-ethyl-4-(trifluoromethyl)-1H-imidazol-2-yl)phenyl)-4,5,6,7-tetrahydropyrazolo[1,5-α]pyridin-4-amine;
(5)-5-(4-(1-(4-(1-ethyl-4-(trifluoromethyl)-1H-imidazol-2-yl)-3-fluorophenyl)ethyl)-5-oxo-4,5,6,7-tetrahydropyrazolo[1,5-α]pyrimidin-2-yl)-1-isopropyl-1H-pyrazole-4-carbonitrile;
(R)-5-(4-(1-(4-(1-ethyl-4-(trifluoromethyl)-1H-imidazol-2-yl)-3-fluorophenyl)ethyl)-5-oxo-4,5,6,7-tetrahydropyrazolo[1,5-α]pyrimidin-2-yl)-1-isopropyl-1H-pyrazole-4-carbonitrile;
2-(4-cyclopropyl-6-methoxypyrimidin-5-yl)-4-(4-(1-ethyl-4-(trifluoromethyl)-1H-imidazol-2-yl)-3-fluorobenzyl)-6,6-dimethyl-4,5,6,7-tetrahydropyrazolo[1,5-α]pyrimidine;
2-(4-(difluoromethyl)-1-isopropyl-1H-pyrazol-5-yl)-4-(4-(1-ethyl-4-(trifluoromethyl)-1H-imidazol-2-yl)-3-fluorobenzyl)-6,7-dihydropyrazolo[1,5-α]pyrimidin-5(4H)-one;
2-(4-(difluoromethoxy)-1-isopropyl-1H-pyrazol-5-yl)-4-(4-(1-ethyl-4-(trifluoromethyl)-1H-imidazol-2-yl)-3-fluorobenzyl)-6,7-dihydropyrazolo[1,5-α]pyrimidin-5(4H)-one;
2-((2-(4-chloro-1-isopropyl-1H-pyrazol-5-yl)-4,5,6,7-tetrahydropyrazolo[1,5-α]pyridin-4-yl)(4-(1-ethyl-4-(trifluoromethyl)-1H-imidazol-2-yl)-3-fluorophenyl)amino)-ethan-1-ol ;
2-(4-cyclopropyl-6-methoxypyrimidin-5-yl)-4-(4-(1-ethyl-4-(trifluoromethyl)-1H-imidazol-2-yl)benzyl)pyrazolo[1,5-α]pyrimidin-5(4H)-one;
(R)-2-(4-cyclopropyl-6-methoxypyrimidin-5-yl)-4-(1-(4-(1-ethyl-4-(trifluoromethyl)-1H-imidazol-2-yl)-3-fluorophenyl)ethyl)pyrazolo[1,5-α]pyrimidin-5(4H)-one;
(S)-2-(4-cyclopropyl-6-methoxypyrimidin-5-yl)-4-(1-(4-(1-ethyl-4-(trifluoromethyl)-1H-imidazol-2-yl)-3-fluorophenyl)ethyl)pyrazolo[1,5-α]pyrimidin-5(4H)-one;
(R)-2-(4-cyclopropyl-6-methoxypyrimidin-5-yl)-4-(1-(4-(1-ethyl-4-(trifluoromethyl)-1H-imidazol-2-yl)phenyl)ethyl)pyrazolo[1,5-α]pyrimidin-5(4H)-one;
(S)-2-(4-cyclopropyl-6-methoxypyrimidin-5-yl)-4-(1-(4-(1-ethyl-4-(trifluoromethyl)-1H-imidazol-2-yl)phenyl)ethyl)pyrazolo[1,5-α]pyrimidin-5(4H)-one;
2-(4-cyclopropyl-6-methoxypyrimidin-5-yl)-4-(4-(1-ethyl-4-(trifluoromethyl)-1H-imidazol-2-yl)benzyl)-[1,2,4]triazolo[1,5-α]pyrimidin-5(4H)-one;
(R)-2-(4-cyclopropyl-6-methoxypyrimidin-5-yl)-4-(1-(4-(1-ethyl-4-(trifluoromethyl)-1H-imidazol-2-yl)phenyl)ethyl)-[1,2,4]triazolo[1,5-α]pyrimidin-5(4H)-one;
(S)-2-(4-cyclopropyl-6-methoxypyrimidin-5-yl)-4-(1-(4-(1-ethyl-4-(trifluoromethyl)-1H-imidazol-2-yl)phenyl)ethyl)-[1,2,4]triazolo[1,5-α]pyrimidin-5(4H)-one;
2-(4-cyclopropyl-6-methoxypyrimidin-5-yl)-4-(1-(4-(1-ethyl-4-(trifluoromethyl)-1H-imidazol-2-yl)phenyl)ethyl)-[1,2,4]triazolo[1,5-α]pyrimidin-5(4H)-one;
(R)-2-(4-cyclopropyl-6-methoxypyrimidin-5-yl)-4-(1-(4-(1-ethyl-4-(trifluoromethyl)-1H-imidazol-2-yl)-3-fluorophenyl)ethyl)-[1,2,4]triazolo[1,5-α]pyrimidin-5(4H)-one;
(S)-2-(4-cyclopropyl-6-methoxypyrimidin-5-yl)-4-(1-(4-(1-ethyl-4-(trifluoromethyl)-1H-imidazol-2-yl)-3-fluorophenyl)ethyl)-[1,2,4]triazolo[1,5-α]pyrimidin-5(4H)-one;
2-(4-chloro-1-isopropyl-1H-pyrazol-5-yl)-4-(4-(1-ethyl-4-(trifluoromethyl)-1H-imidazol-2-yl)-3-fluorobenzyl)-[1,2,4]triazolo[1,5-α]pyrimidin-5(4H)-one;
2-(4-cyclopropyl-6-(difluoromethoxy)pyrimidin-5-yl)-4-(4-(1-isopropyl-4-(trifluoromethyl)-1H-imidazol-2-yl)benzyl)-[1,2,4]triazolo[1,5-α]pyrimidin-5(4H)-one;
2-(4-cyclopropyl-6-(difluoromethoxy)pyrimidin-5-yl)-4-(4-(1-ethyl-4-(trifluoromethyl)-1H-imidazol-2-yl)benzyl)-[1,2,4]triazolo[1,5-α]pyrimidin-5(4H)-one;
2-(4-cyclopropyl-6-methoxypyrimidin-5-yl)-4-(3-fluoro-4-(1-isopropyl-4-(trifluoromethyl)-1H-imidazol-2-yl)-2-methoxybenzyl)-[1,2,4]triazolo[1,5-α]pyrimidin-5(4H)-one;
2-(4-cyclopropyl-6-methoxypyrimidin-5-yl)-4-(4-(1-ethyl-4-(trifluoromethyl)-1H-imidazol-2-yl)-3-fluorobenzyl)-6-methyl-6,7-dihydro-[1,2,4]triazolo[1,5-α]pyrimidin-5(4H)-one;
(R)-2-(4-cyclopropyl-6-methoxypyrimidin-5-yl)-4-(4-(1-ethyl-4-(trifluoromethyl)-1H-imidazol-2-yl)-3-fluorobenzyl)-6-methyl-6,7-dihydro-[1,2,4]triazolo[1,5-α]pyrimidin-5(4H)-one;
(S)-2-(4-cyclopropyl-6-methoxypyrimidin-5-yl)-4-(4-(1-ethyl-4-(trifluoromethyl)-1H-imidazol-2-yl)-3-fluorobenzyl)-6-methyl-6,7-dihydro-[1,2,4]triazolo[1,5-α]pyrimidin-5(4H)-one;
2-(4-chloro-1-isopropyl-1H-pyrazol-5-yl)-4-(4-(1-ethyl-4-(trifluoromethyl)-1H-imidazol-2-yl)-3-fluorobenzyl)-6-methyl-6,7-dihydro-[1,2,4]triazolo[1,5-α]pyrimidin-5(4H)-one;
2-(4-cyclopropyl-6-(difluoromethoxy)pyrimidin-5-yl)-4-(4-(1-ethyl-4-(trifluoromethyl)-1H-imidazol-2-yl)-3-fluorobenzyl)-6-methyl-6,7-dihydro-[1,2,4]triazolo[1,5-α]pyrimidin-5(4H)-one;
2-(4-chloro-1-isopropyl-1H-pyrazol-5-yl)-N-(4-(1-ethyl-4-(trifluoromethyl)-1H-imidazol-2-yl)phenyl)-5,5-dimethyl-4,5,6,7-tetrahydropyrazolo[1,5-α]pyridin-4-amine;
2-(4-chloro-1-isopropyl-1H-pyrazol-5-yl)-4-(4-(1-ethyl-4-(trifluoromethyl)-1H-imidazol-2-yl)-3-fluorobenzyl)-4,5,6,7-tetrahydropyrazolo[1,5-α]pyrimidin-6-amine;
2-(4-chloro-1-isopropyl-1H-pyrazol-5-yl)-4-((5-(1-ethyl-4-(trifluoromethyl)-1H-imidazol-2-yl)pyrimidin-2-yl)methyl)-6,7-dihydropyrazolo[1,5-α]pyrimidin-5(4H)-one;
2-(4-chloro-1-isopropyl-1H-pyrazol-5-yl)-4-(4-(1-ethyl-4-(trifluoromethyl)-1H-imidazol-2-yl)benzyl)-4,5,6,7-tetrahydropyrazolo[1,5-α]pyrazine;
2-(4-chloro-1-isopropyl-1H-pyrazol-5-yl)-4-(4-(1-ethyl-4-(trifluoromethyl)-1H-imidazol-2-yl)benzyl)-5-methyl-4,5,6,7-tetrahydropyrazolo[1,5-α]pyrazine;
2-(4-cyclopropyl-6-methoxypyrimidin-5-yl)-4-(4-(1-ethyl-4-(trifluoromethyl)-1H-imidazol-2-yl)benzyl)-4,5,6,7-tetrahydropyrazolo[1,5-α]pyrazine;
2-(4-cyclopropyl-6-methoxypyrimidin-5-yl)-4-(4-(1-ethyl-4-(trifluoromethyl)-1H-imidazol-2-yl)benzyl)-5-methyl-4,5,6,7-tetrahydropyrazolo[1,5-α]pyrazine;
(R)-2-(4-cyclopropyl-6-methoxypyrimidin-5-yl)-4-(1-(4-(1-isopropyl-4-(trifluoromethyl)-1H-imidazol-2-yl)phenyl)ethyl)-6,7-dihydropyrazolo[1,5-α]pyrimidin-5(4H)-one;
2-(4-cyclopropyl-6-(difluoromethoxy)pyrimidin-5-yl)-4-(4-(1-ethyl-4-(trifluoromethyl)-1H-imidazol-2-yl)benzyl)-6,7-dihydro-[1,2,4]triazolo[1,5-α]pyrimidin-5(4H)-one;
2-(4-cyclopropyl-6-(difluoromethoxy)pyrimidin-5-yl)-4-(3-fluoro-4-(1-isopropyl-4-(trifluoromethyl)-1H-imidazol-2-yl)-2-methoxybenzyl)-6,7-dihydro-[1,2,4]triazolo[1,5-α]pyrimidin-5(4H)-one;
2-(4-cyclopropyl-6-(difluoromethoxy)pyrimidin-5-yl)-4-(4-(1-(1-fluoropropan-2-yl)-4-(trifluoromethyl)-1H-imidazol-2-yl)benzyl)-6,7-dihydro-[1,2,4]triazolo[1,5-α]pyrimidin-5(4H)-one;
2-(4-cyclopropyl-6-(difluoromethoxy)pyrimidin-5-yl)-4-(4-(1-(2-fluoroethyl)-4-(trifluoromethyl)-1H-imidazol-2-yl)benzyl)-6,7-dihydro-[1,2,4]triazolo[1,5-α]pyrimidin-5(4H)-one;
2-(4-cyclopropyl-6-(difluoromethoxy)pyrimidin-5-yl)-4-(4-(1-methyl-4-(trifluoromethyl)-1H-imidazol-2-yl)benzyl)-6,7-dihydro-[1,2,4]triazolo[1,5-α]pyrimidin-5(4H)-one;
2-(4-cyclopropyl-6-(difluoromethoxy)pyrimidin-5-yl)-4-(4-(1-isopropyl-4-(trifluoromethyl)-1H-imidazol-2-yl)benzyl)-6,7-dihydro-[1,2,4]triazolo[1,5-α]pyrimidin-5(4H)-one;
2-(4-cyclopropyl-6-(difluoromethoxy)pyrimidin-5-yl)-4-(4-(1-ethyl-4-(trifluoromethyl)-1H-imidazol-2-yl)-3-fluorobenzyl)-6,7-dihydro-[1,2,4]triazolo[1,5-α]pyrimidin-5(4H)-one;
2-(4-chloro-1-isopropyl-1H-pyrazol-5-yl)-4-(2,2,2-trifluoro-1-(3-fluoro-4-(1-isopropyl-4-(trifluoromethyl)-1H-imidazol-2-yl)phenyl)ethyl)-4,5,6,7-tetrahydropyrazolo[1,5-α]pyrimidine;
2-(4-cyclopropyl-6-methoxypyrimidin-5-yl)-4-(1-(4-(5-methoxy-3-(trifluoromethyl)-1H-pyrazol-1-yl)phenyl)ethyl)-6,7-dihydro-[1,2,4]triazolo[1,5-α]pyrimidin-5(4H)-one;
2-(4-cyclopropyl-2-(difluoromethoxy)pyridin-3-yl)-4-(4-(1-ethyl-4-(trifluoromethyl)-1H-imidazol-2-yl)benzyl)-6,7-dihydro-[1,2,4]triazolo[1,5-α]pyrimidin-5(4H)-one;
2-(4-cyclopropyl-6-(difluoromethoxy)pyrimidin-5-yl)-4-(4-(1-(propan-2-yl-d7)-4-(trifluoromethyl)-1H-imidazol-2-yl)benzyl)-6,7-dihydro-[1,2,4]triazolo[1,5-α]pyrimidin-5(4H)-one;
2-(4-cyclopropyl-6-(difluoromethoxy)pyrimidin-5-yl)-4-(4-(1-(ethyl-d₅)-4-(trifluoromethyl)-1H-imidazol-2-yl)benzyl)-6,7-dihydro-[1,2,4]triazolo[1,5-α]pyrimidin-5(4H)-one;
2-(4-(difluoromethoxy)-6-methoxypyrimidin-5-yl)-4-(4-(1-ethyl-4-(trifluoromethyl)-1H-imidazol-2-yl)benzyl)-6,7-dihydro-[1,2,4]triazolo[1,5-α]pyrimidin-5(4H)-one;
2-(4-cyclopropyl-6-(difluoromethoxy)pyrimidin-5-yl)-4-(3-fluoro-4-(1-methyl-4-(trifluoromethyl)-1H-imidazol-2-yl)benzyl)-6,7-dihydro-[1,2,4]triazolo[1,5-α]pyrimidin-5(4H)-one;
2-(4-cyclopropyl-6-methoxypyrimidin-5-yl)-4-(1-(4-(1-methyl-4-(trifluoromethyl)-1H-imidazol-2-yl)phenyl)ethyl)-6,7-dihydro-[1,2,4]triazolo[1,5-α]pyrimidin-5(4H)-one;
2-(4-cyclopropyl-6-methoxypyrimidin-5-yl)-4-(1-(4-(1-(2-fluoroethyl)-4-(trifluoromethyl)-1H-imidazol-2-yl)phenyl)ethyl)-6,7-dihydro-[1,2,4]triazolo[1,5-α]pyrimidin-5(4H)-one;
2-(4-cyclopropyl-6-methoxypyrimidin-5-yl)-4-(1-(4-(1-ethyl-4-(trifluoromethyl)-1H-imidazol-2-yl)-3-fluoro-2-methoxyphenyl)ethyl)-6,7-dihydro-[1,2,4]triazolo[1,5-α]pyrimidin-5(4H)-one;
(S)-2-(4-cyclopropyl-6-methoxypyrimidin-5-yl)-4-(4-(1-ethyl-4-(trifluoromethyl)-1H-imidazol-2-yl)benzyl)-6-methyl-6,7-dihydro-[1,2,4]triazolo[1,5-α]pyrimidin-5(4H)-one;
(R)-2-(4-cyclopropyl-6-methoxypyrimidin-5-yl)-4-(4-(1-ethyl-4-(trifluoromethyl)-1H-imidazol-2-yl)benzyl)-6-methyl-6,7-dihydro-[1,2,4]triazolo[1,5-α]pyrimidin-5(4H)-one;
2-(4-cyclopropyl-6-methoxypyrimidin-5-yl)-4-(4-(1-(1-fluoropropan-2-yl)-4-(trifluoromethyl)-1H-imidazol-2-yl)benzyl)-6-methyl-6,7-dihydro-[1,2,4]triazolo[1,5-α]pyrimidin-5(4H)-one;
2-(4-cyclopropyl-6-methoxypyrimidin-5-yl)-4-(1-(4-(1-ethyl-4-(trifluoromethyl)-1H-imidazol-2-yl)phenyl)ethyl)-6-methyl-6,7-dihydro-[1,2,4]triazolo[1,5-α]pyrimidin-5(4H)-one;
2-(4-cyclopropyl-6-(difluoromethoxy)pyrimidin-5-yl)-4-(3-fluoro-4-(1-methyl-4-(trifluoromethyl)-1H-imidazol-2-yl)benzyl)-[1,2,4]triazolo[1,5-α]pyrimidin-5(4H)-one;
2-(4-cyclopropyl-6-methoxypyrimidin-5-yl)-4-(3-fluoro-4-(1-methyl-4-(trifluoromethyl)-1H-imidazol-2-yl)benzyl)-[1,2,4]triazolo[1,5-α]pyrimidin-5(4H)-one;
2-(4-cyclopropyl-6-methoxypyrimidin-5-yl)-N-(4-(1-(2-fluoroethyl)-4-(trifluoromethyl)-1H-imidazol-2-yl)phenyl)-4,5,6,7-tetrahydropyrazolo[1,5-α]pyridin-4-amine;
2-(4-cyclopropyl-6-methoxypyrimidin-5-yl)-N-(3-fluoro-4-(1-methyl-4-(trifluoromethyl)-1H-imidazol-2-yl)phenyl)-4,5,6,7-tetrahydropyrazolo[1,5-α]pyridin-4-amine;
2-(4-cyclopropyl-6-methoxypyrimidin-5-yl)-N-(4-(1-(ethyl-d₅)-4-(trifluoromethyl)-1H-imidazol-2-yl)phenyl)-4,5,6,7-tetrahydropyrazolo[1,5-α]pyridin-4-amine;
2-(4-chloro-1-isopropyl-1H-pyrazol-5-yl)-N-(4-(1-(1-fluoropropan-2-yl)-4-(trifluoromethyl)-1H-imidazol-2-yl)phenyl)-4,5,6,7-tetrahydropyrazolo[1,5-α]pyridin-4-amine;
2-(4-cyclopropyl-6-methoxypyrimidin-5-yl)-4-(4-(1-ethyl-4-(trifluoromethyl)-1H-imidazol-2-yl)benzyl)-6-methyl-[1,2,4]triazolo[1,5-α]pyrimidin-5(4H)-one;
2-(4-cyclopropyl-6-(difluoromethoxy)pyrimidin-5-yl)-4-(4-(1-ethyl-4-(trifluoromethyl)-1H-imidazol-2-yl)benzyl)-6-methyl-[1,2,4]triazolo[1,5-α]pyrimidin-5(4H)-one; and
(4S)-2-(4-cyclopropyl-6-methoxypyrimidin-5-yl)-4-(4-(1-(1-fluoropropan-2-yl)-4-(trifluoromethyl)-1H-imidazol-2-yl)phenoxy)-4,5,6,7-tetrahydropyrazolo[1,5-α]pyridine.

37. The compound of any one of claims 1-36, wherein the compound inhibits a USP1 protein.

38. The compound of any one of claims 1-36, wherein the compound inhibits a USP1 protein with an IC₅₀ value of less than about 1 μM in a Ub-Rho deubiquitinating assay.

39. The compound of claim 38, wherein the Ub-Rho deubiquitinating assay is the assay disclosed in Example 394.

40. A method of treating cancer in a patient comprising administering to the patient a therapeutically effective amount of the compound of any one of claims 1-39.

41. A pharmaceutical composition comprising the compound of any one of claims 1-39, or a pharmaceutically acceptable salt or solvate thereof, and a pharmaceutically acceptable carrier.

42. The pharmaceutical composition of claim 41 for use in treatment of cancer.

43. The compound of any one of claims 1-39, or a pharmaceutically acceptable salt or solvate thereof, for use in treatment of cancer.

44. Use of the compound of any one of claims 1-39, or a pharmaceutically acceptable salt or solvate thereof, for the manufacture of a medicament for treatment of cancer.

45. A kit comprising the compound of any one of claims 1-39, or a pharmaceutically acceptable salt or solvate thereof, or the pharmaceutical composition of any one of claims 41-42 and instructions for administering the compound, or a pharmaceutically acceptable salt or solvate thereof, or the pharmaceutical composition to a patient having cancer.

46. A method of treating cancer in a patient comprising administering to the patient the compound of any one of claims 1-39, or a pharmaceutically acceptable salt or solvate thereof or the pharmaceutical composition of any one of claims 41-42.

47. The method of any one of claims 40 and 46, the pharmaceutical composition of claim 42, the compound of claim 43, the use of claim 44, or the kit of claim 45, wherein the cancer is selected from the group consisting of a hematological cancer, a lymphatic cancer, a DNA damage repair pathway deficient cancer, a homologous-recombination deficient cancer, a cancer comprising cancer cells with a mutation in a gene encoding p53, and a cancer comprising cancer cells with a loss of function mutation in a gene encoding p53.

48. The method of any one of claims 40 and 46, the pharmaceutical composition of claim 42, the compound of claim 43, the use of claim 44, or the kit of claim 45, wherein the cancer is selected from the group consisting of lung cancer, non-small cell lung cancer (NSCLC), colon cancer, bladder cancer, osteosarcoma, ovarian cancer, skin cancer, and breast cancer.

49. The method of any one of claims 40 and 46, the pharmaceutical composition of claim 42, the compound of claim 43, the use of claim 44, or the kit of claim 45, wherein the cancer is ovarian cancer or breast cancer.

50. The method of any one of claims 40 and 46, the pharmaceutical composition of claim 42, the compound of claim 43, the use of claim 44, or the kit of claim 45, wherein the cancer is ovarian cancer.

51. The method of any one of claims 40 and 46, the pharmaceutical composition of claim 42, the compound of claim 43, the use of claim 44, or the kit of claim 45, wherein the cancer is a breast cancer.

52. The method of any one of claims 40 and 46, the pharmaceutical composition of claim 42, the compound of claim 43, the use of claim 44, or the kit of claim 45, wherein the cancer is a triple negative breast cancer.

53. The method of any one of claims 40 and 46-52, the pharmaceutical composition of claim 42, the compound of claim 43, the use of claim 44, or the kit of claim 45, wherein the cancer comprises cancer cells with elevated levels of RAD18.

54. The method, pharmaceutical composition, compound, use, or kit of claim 53, wherein the elevated levels of RAD18 are elevated RAD18 protein levels.

55. The method, pharmaceutical composition, compound, use, or kit of claim 53, wherein the elevated levels of RAD18 are elevated RAD18 mRNA levels.

56. The method of any one of claims 53-55, wherein the elevated levels of RAD18 have been detected prior to the administration.

57. The method of claim 56, further comprising detecting RAD18 levels in a cancer sample obtained from the subject.

58. The method of any one of claims 40 and 46, the pharmaceutical composition of claim 42, the compound of claim 43, the use of claim 44, or the kit of claim 45, wherein the cancer is a DNA damage repair pathway deficient cancer.

59. The method, pharmaceutical composition, compound, use, or kit of claim 58, wherein the cancer is a homologous-recombination deficient cancer.

60. The method of any one of claims 40 and 46, the pharmaceutical composition of claim 42, the compound of claim 43, the use of claim 44, or the kit of claim 45, wherein the cancer comprises cancer cells with a mutation in a gene encoding p53, optionally wherein the mutation in the gene encoding p53 is a loss of function mutation.

61. The method of any one of claims 40, 46, and 48-57, the pharmaceutical composition of claim 42, the compound of claim 43, the use of claim 44, or the kit of claim 45, wherein the cancer is a BRCA1 mutant cancer.

62. The method of any one of claims 40, 46, and 48-57, the pharmaceutical composition of claim 42, the compound of claim 43, the use of claim 44, or the kit of claim 45, wherein the cancer is a BRCA2 mutant cancer.

63. The method of any one of claims 40, 46, and 48-57, the pharmaceutical composition of claim 42, the compound of claim 43, the use of claim 44, or the kit of claim 45, wherein the cancer is a BRCA1 mutant cancer and a BRCA2 mutant cancer.

64. The method of any one of claims 40, 46, and 48-57, the pharmaceutical composition of claim 42, the compound of claim 43, the use of claim 44, or the kit of claim 45, wherein the cancer is a PARP inhibitor resistant or refractory cancer or a PARP inhibitor resistant or refractory BRCA1 or BRCA2 mutant cancer.

65. A method of treating a USP1 protein mediated disorder comprising administering to a patient in need thereof a compound of any one of claims 1-39, or a pharmaceutically acceptable salt or solvate thereof, or the pharmaceutical composition of claim 41 in an effective amount to treat the USP1 protein mediated disorder.

66. The method of any one of claims 40 and 46-63, the compound of any one of claims 38-39 and 43, the use of claim 44, the kit of claim 45, or the pharmaceutical composition of any one of claims 41-42, wherein the USP1 protein comprises the amino acid sequence of SEQ ID NO:1.

67. A method of inhibiting a USP1 protein comprising contacting a USP1 protein with the compound of any one of claims 1-39, or a pharmaceutically acceptable salt or solvate thereof, or the pharmaceutical composition of claim 41.

68. The method of claim 57, wherein the contacting occurs in vitro.

69. The method of claim 57, wherein the contacting occurs in vivo.

70. The method, pharmaceutical composition, compound, use, or the kit of claim 64, wherein the BRCA1 or BRCA2 mutant cancer is a BRCA1 or BRCA2 deficient cancer.
